(19) United States
(12) Patent Application Publication
(10) Pub. No.: US 2024/0279220 A1
(43) Pub. Date: Aug. 22, 2024

(54) INDOLE COMPOUNDS AND METHODS OF USE

(71) Applicant: Genzyme Corporation, Cambridge, MA (US)

(72) Inventors: Junkai Liao, Andover, MA (US); Mark Munson, Acton, MA (US); Sukanthini Thurairatnam, Cambridge, MA (US); Bradford Hirth, Cambridge, MA (US); Zhongli Gao, Waltham, MA (US); Gregory Hurlbut, Boxborough, MA (US); David Borcherding, Cambridge, MA (US); Matthieu Barrague, Cambridge, MA (US); Timothy Alan Gillespy, Cambridge, CA (US); Alexandre Gross, Cambridge, CA (US); Andrew Good, Cambridge, MA (US); Roy Vaz, Cambridge, MA (US); Jinyu Liu, Cambridge, MA (US); Yi Li, Cambridge, MA (US); Markus Metz, Cambridge, MA (US); Anatoly Ruvinsky, Cambridge, MA (US); Claude Barberis, Cambridge, CA (US)

(21) Appl. No.: 18/593,686

(22) Filed: Mar. 1, 2024

Related U.S. Application Data
(63) Continuation of application No. PCT/US2022/075945, filed on Sep. 2, 2022.
(60) Provisional application No. 63/240,765, filed on Sep. 3, 2021.

(51) Int. Cl. C07D 417/14; A61K 31/404; A61K 31/4155; A61K 31/4178; A61K 31/4196; A61K 31/422; A61K 31/427; A61K 31/4439; A61K 31/675; C07D 401/14; C07D 403/12; C07D 405/14; C07D 409/14; C07D 413/12; C07D 417/12; C07F 9/6503

(52) U.S. Cl. CPC C07D 417/14; A61K 31/404; A61K 31/4155; A61K 31/4178; A61K 31/4196; A61K 31/422; A61K 31/427; A61K 31/4439; A61K 31/675; C07D 401/14; C07D 403/12; C07D 405/14; C07D 409/14; C07D 413/12; C07D 417/12; C07F 9/65031

(57) ABSTRACT

The invention relates to heterocyclic compounds, pharmaceutically acceptable salts thereof, and pharmaceutical preparations thereof. Also described herein are compositions and the use of such compounds in methods of treating diseases and conditions mediated by deficient CFTR activity, in particular cystic fibrosis.

### CROSS-REFERENCE TO RELATED APPLICATIONS

This application is a continuation of International Application No. PCT/US2022/075945, filed Sep. 2, 2022, which claims priority to U.S. Provisional Application No. 63/240,765, filed Sep. 3, 2021, the contents of which are incorporated herein by reference.

### BACKGROUND

Cystic fibrosis (CF), an autosomal recessive disorder, is caused by functional deficiency of the cAMP-activated plasma membrane chloride channel, cystic fibrosis transmembrane conductance regulator (CFTR), which results in pulmonary and other complications. The gene encoding CFTR has been identified and sequenced (See Gregory, R. J. et al. (1990) Nature 347:382-386; Rich, D. P. et al. (1990) Nature 347:358-362), (Riordan, J. R. et al. (1989) Science 245:1066-1073). CFTR, a member of the ATP binding cassette (ABC) superfamily is composed of two six membrane-spanning domains (MSD1 and MSD2), two nucleotide bind domains (NBD1 and NBD2), a regulatory region (R) and four cytosolic loops (CL1-4). CFTR protein is located primarily in the apical membrane of epithelial cells where it functions to conduct anions, including chloride, bicarbonate and thiocyanate into and out of the cell. CFTR may have a regulatory role over other electrolyte channels, including the epithelial sodium channel ENaC.

In cystic fibrosis patients, the absence or dysfunction of CFTR leads to exocrine gland dysfunction and a multisystem disease, characterized by pancreatic insufficiency and malabsorption, as well as abnormal mucociliary clearance in the lung, mucostasis, chronic lung infection and inflammation, decreased lung function and ultimately respiratory failure. While more than 1,900 mutations have been identified in the CFTR gene, a detailed understanding of how each CFTR mutation may impact channel function is known for only a few. (Derichs, European Respiratory Review, 22:127, 58-65 (2013)). The most frequent CFTR mutation is the in-frame deletion of phenylalanine at residue 508 (ΔF508) in the first nucleotide binding domain (NBD1). Over 80% of cystic fibrosis patients have the deletion at residue 508 in at least one allele. The loss of this key phenylalanine renders NBD1 conformationally unstable at physiological temperature and compromises the integrity of the interdomain interface between NDB1 and CFTR's second transmembrane domain (ICL4).

The ΔF508 mutation causes production of misfolded CFTR protein which, rather than traffic to the plasma membrane, is instead retained in the endoplasmic reticulum and targeted for degradation by the ubiquitin-proteasome system.

The loss of a functional CFTR channel at the plasma membrane disrupts ionic homeostasis and airway surface hydration leading to reduced lung function. Reduced periciliary liquid volume and increased mucus viscosity impede mucociliary clearance resulting in chronic infection and inflammation. In the lung, the loss of CFTR-function leads to numerous physiological effects downstream of altered anion conductance that result in the dysfunction of additional organs such as the pancreas, intestine and gall bladder.

By studying the mechanistic aspects of CFTR misfolding and corrections, small molecules have been identified as CF modulators, that can act as correctors and/or potentiators.

Despite the identification of compounds that modulate CFTR, there is no cure for this fatal disease and identification of new compounds and new methods of therapy are needed as well as new methods for treating or lessening the severity of cystic fibrosis and other CFTR mediated conditions and diseases in a patient.

### SUMMARY

Disclosed herein are compounds of Formula I:

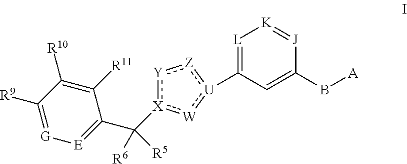

wherein
A is selected from

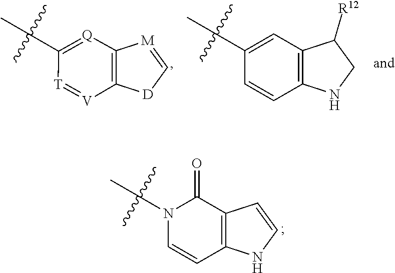

B is O or C═O;
M is CR¹³ or N;
Q is CR¹ or N;
T is CR² or N;
V is CR³ or N;
D is NR¹⁴ or S;
J is CR^{4a} or N;
K is CR^{4b} or N;
L is CR^{4c} or N;
E is CR⁷ or N;
G is CR⁸ or N;
U is N or C;
W is N, NR^{a}, O or S;
X is N or C;
Y is C, CR^{b}, N or NR^{c};
Z is N, NR^{d}, O, S, or CH;
[symbol] is a single or double bond;
R^{a} is H, halo, alkyl, alkoxy, or aralkyl;
R^{b}, R^{c}, and R^{d} are each independently H, halo, or alkyl;
R¹ is selected from H, D, halo, CN, nitro, alkyl, alkenyl, alkynyl, alkoxy, hydroxy, amido, cycloalkyl, aryl, heterocyclyl, heteroaryl, alkylcarbonyl, carboxy, alkoxycarbonyl, amino, amido, phosphino, thioalkyl, sulfinyl, sulfonyl, and sulfonamido;
R² is selected from H, halo, CN, alkyl, alkoxy, hydroxy, amino, alkoxycarbonyl, and amido;
R³ is selected from H, halo, CN, and alkyl;
R^{4a}, R^{4b}, and R^{4c} are each independently selected from H, halo, CN, alkyl, alkoxy, hydroxy, amino, alkoxycarbonyl, and amido;
R⁵ is selected from H, halo, CN, alkyl, alkenyl, alkynyl, hydroxy, alkoxy, alkylcarbonyl, alkoxycarbonyl, amino, and amido;
R⁶ is H, CN or alkyl; or
R⁵ and R⁶ together with the carbon atoms to which they are attached form a vinyl, carbonyl, cycloalkyl or heterocyclyl;
R⁷ and R¹¹ are each independently selected from H, halo, CN, alkyl, alkoxy, aryl, and heteroaryl; or
R⁶ and R⁷ together with the carbon atoms to which they are attached form a cycloalkyl;
R⁸ and R¹⁰ are each independently selected from H, halo, alkyl, alkenyl, alkynyl, alkoxy, amido, carboxy, alkoxycarbonyl, thioalkyl, cycloalkyl, and heterocyclyl; or
R⁷ and R⁸ together with the carbon atoms to which they are attached form a cycloalkyl;
R⁹ is selected from H, halo, alkyl, alkynyl, and alkoxy; or
R⁸ and R⁹ together with the carbon atoms to which they are attached form a cycloalkyl, heteroaryl, or heterocyclyl; or
R⁹ and R¹⁰ together with the carbon atoms to which they are attached form a cycloalkyl;
R¹² is H, deuterium or carboxy;
R¹³ is H, deuterium, halo or thioalkyl;
R¹⁴ is H, sulfonyl, carbonyl or phosphate;
wherein each alkyl, alkenyl, alkynyl, alkoxy, amino, amido, alkylcarbonyl, alkoxycarbonyl, cycloalkyl, heterocyclyl, aryl, and heteroaryl is independently unsubstituted or substituted with one or more substituents selected from deuterium, halo, CN, nitro, hydroxy, alkyl, amino, amido, oxo, boronate, cycloalkyl, aryl, heterocyclyl, heteroaryl, carbonyl, carboxy, acyloxy, alkoxy, thio, phosphino, phosphono, phosphate, sulfinyl, sulfonyl, sulfonamido, ureido, amidino, hydroxyamidino, iminosulfanoyl, 2-tetrahydrothiophene-1,1-dioxide, and isothiazolidine-1,1-dioxide; and
wherein at least one of R⁷, R⁸, R⁹, R¹⁰, and R¹¹ is not H.

Disclosed herein are methods for treating a disease or condition mediated by deficient CFTR activity. Such diseases and conditions include, but are not limited to, cystic fibrosis, congenital bilateral absence of vas deferens (CBAVD), acute, recurrent, or chronic pancreatitis, disseminated bronchiectasis, asthma, allergic pulmonary aspergillosis, chronic obstructive pulmonary disease (COPD), chronic sinusitis, dry eye disease, protein C deficiency, abetalipoproteinemia, lysosomal storage disease, type 1 chylomicronemia, mild pulmonary disease, lipid processing deficiencies, type 1 hereditary angioedema, coagulation-fibrinolyis, hereditary hemochromatosis, CFTR-related metabolic syndrome, chronic bronchitis, constipation, pancreatic insufficiency, hereditary emphysema, and Sjogren's syndrome. In some embodiments, the disease is cystic fibrosis.

In certain embodiments, the present invention provides a pharmaceutical composition suitable for use in a subject in the treatment or prevention of disease and conditions associate with deficient CFTR activity, comprising any of the compounds described herein (e.g., a compound of the invention, such as a compound of formula I, and one or more pharmaceutically acceptable carriers or excipients. In certain embodiments, the pharmaceutical preparations may be for use in treating or preventing a condition or disease as described herein.

Provided herein are combination therapies of compounds of formula I with CFTR-active agents that can enhance the therapeutic benefit beyond the ability of the primary therapy alone.

### BRIEF DESCRIPTION OF THE FIGURES

FIG. 1 shows compounds of Formula AA that can be used as additional therapeutics.

FIG. 2 shows compounds of Formula BB that can be used as additional therapeutics.

FIG. 3 shows compounds of Formula CC that can be used as additional therapeutics.

### DETAILED DESCRIPTION

The present invention provides compounds of Formula I:

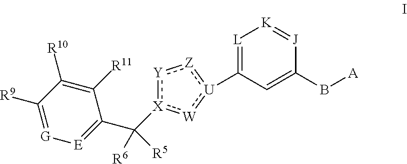

wherein
A is selected from

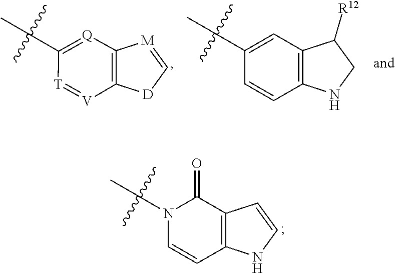

B is O or C═O;
M is CR¹³ or N;
Q is CR¹ or N;
T is CR² or N;
V is CR³ or N;
D is NR¹⁴ or S;
J is CR^{4a} or N;
K is CR^{4b} or N;
L is CR^{4c} or N;
E is CR⁷ or N;
G is CR⁸ or N;
U is N or C;
W is N, NR^{a}, O or S;
X is N or C;
Y is CR^{b}, N or NR^{c};
Z is N, NR^{d}, O, S, or CH;
[symbol] is a single or double bond;
R^{a}, R^{c}, and R^{d} are each independently selected from H, alkyl, or aralkyl;
R^{b} is selected from H, halo, or alkyl;
R¹ is selected from H, D, halo, CN, nitro, alkyl, alkenyl, alkynyl, alkoxy, hydroxy, amido, cycloalkyl, aryl, heterocyclyl, heteroaryl, alkylcarbonyl, carboxy, alkoxycarbonyl, amino, amido, phosphino, thioalkyl, sulfinyl, sulfonyl, sulfonimidolyl, phosphoryl, sulfonamido, and isothiazolidine-1,1-dioxide;
R² is selected from H, halo, CN, alkyl, alkoxy, hydroxy, amino, alkoxycarbonyl, and amido;
R³ is selected from H, halo, CN, and alkyl;
R^{4a}, R^{4b}, and R^{4c} are each independently selected from H, halo, CN, alkyl, alkoxy, hydroxy, amino, alkoxycarbonyl, and amido;
R⁵ is selected from H, halo, CN, alkyl, alkenyl, alkynyl, hydroxy, alkoxy, alkylcarbonyl, alkoxycarbonyl, amino, carboxy, and amido;
R⁶ is H, CN or alkyl; or
R⁵ and R⁶ together with the carbon atoms to which they are attached form a vinyl, carbonyl, cycloalkyl or heterocyclyl;
R⁷ and R¹¹ are each independently selected from H, halo, CN, alkyl, alkoxy, aryl, and heteroaryl; or
R⁶ and R⁷ together with the carbon atoms to which they are attached form a cycloalkyl;
R⁸ and R¹⁰ are each independently selected from H, halo, alkyl, alkenyl, alkynyl, alkoxy, amido, carboxy, alkoxycarbonyl, thioalkyl, cycloalkyl, and heterocyclyl; or
R⁷ and R⁸ together with the carbon atoms to which they are attached form a cycloalkyl;
R⁹ is selected from H, halo, alkyl, alkynyl, and alkoxy; or
R⁸ and R⁹ together with the carbon atoms to which they are attached form a cycloalkyl, heteroaryl, or heterocyclyl; or
R⁹ and R¹⁰ together with the carbon atoms to which they are attached form a cycloalkyl;
R¹² is H, deuterium or carboxy;
R¹³ is H, deuterium, halo or thioalkyl;
R¹⁴ is H, sulfonyl, carbonyl or phosphate;
wherein each alkyl, alkenyl, alkynyl, alkoxy, amino, amido, alkylcarbonyl, alkoxycarbonyl, cycloalkyl, heterocyclyl, aryl, and heteroaryl is independently unsubstituted or substituted with one or more substituents selected from deuterium, halo, CN, nitro, hydroxy, alkyl, amino, amido, oxo, boronate, cycloalkyl, aryl, heterocyclyl, heteroaryl, carbonyl, carboxy, acyloxy, alkoxy, thio, phosphino, phosphono, phosphate, sulfinyl, sulfonyl, sulfonamido, ureido, amidino, hydroxyamidino, iminosulfanoyl, 2-tetrahydrothiophene-1,1-dioxide, and isothiazolidine-1,1-dioxide; and
wherein at least one of R⁷, R⁸, R⁹, R¹⁰, and R¹¹ is not H; and provided that when E is CR⁷, G is CR⁸, and one of R⁷, R⁸, R⁹, R¹⁰, and R¹¹ is unsubstituted alkyl, halo, or haloalkyl, then the ring to which R⁷, R⁸, R⁹, R¹⁰, and R¹¹ are attached is disubstituted.

The present invention provides compounds of Formula I′:

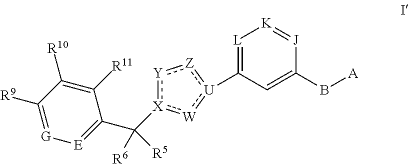

wherein
A is selected from

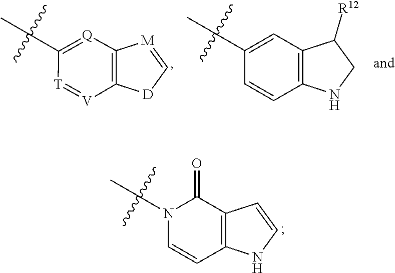

B is selected from O, C═O, C(R^{e})₂, N, S, SO, and SO₂;
M is CR¹³ or N;
Q is CR¹ or N;
T is CR² or N;
V is CR³ or N;
D is NR¹⁴ or S;
J is CR^{4a} or N;
K is CR^{4b} or N;
L is CR^{4c} or N;
E is CR⁷ or N;
G is CR⁸ or N;
U is N or C;
W is N, NR^{a}, O or S;
X is N or C;
Y is CR^{b}, N or NR^{c};
Z is N, NR^{d}, O, S, CH or C-alkyl;
[symbol] is a single or double bond;
R^{a}, R^{c}, and R^{d} are each independently selected from H, alkyl, or aralkyl;
R^{b} is selected from H, halo, or alkyl;
each R^{e} is independently selected from H, halo and hydroxy, wherein both values of R^{e} are not simultaneously H;
R¹ is selected from H, D, halo, CN, nitro, alkyl, alkenyl, alkynyl, alkoxy, hydroxy, amido, cycloalkyl, aryl, heterocyclyl, heteroaryl, alkylcarbonyl, carboxy, alkoxycarbonyl, amino, amido, phosphino, thioalkyl, sulfinyl, sulfonyl, sulfonimidolyl, phosphoryl, sulfonamido, and isothiazolidine-1,1-dioxide;
R² is selected from H, halo, CN, alkyl, alkoxy, hydroxy, amino, alkoxycarbonyl, and amido;
R³ is selected from H, halo, CN, and alkyl;
R^{4a}, R^{4b}, and R^{4c} are each independently selected from H, halo, CN, alkyl, alkoxy, hydroxy, amino, alkoxycarbonyl, and amido;
R⁵ is selected from H, halo, CN, alkyl, alkenyl, alkynyl, hydroxy, alkoxy, alkylcarbonyl, alkoxycarbonyl, amino, carboxy, and amido;
R⁶ is H, CN or alkyl; or
R⁵ and R⁶ together with the carbon atoms to which they are attached form a vinyl, carbonyl, carboxy, cycloalkyl or heterocyclyl;
R⁷ and R¹¹ are each independently selected from H, halo, CN, alkyl, alkoxy, aryl, and heteroaryl; or
R⁶ and R⁷ together with the carbon atoms to which they are attached form a cycloalkyl or heterocyclyl;
R⁸ and R¹⁰ are each independently selected from H, halo, alkyl, alkenyl, alkynyl, alkoxy, amido, carboxy, alkoxycarbonyl, thioalkyl, cycloalkyl, and heterocyclyl; or
R⁷ and R⁸ together with the carbon atoms to which they are attached form a cycloalkyl or a heterocyclyl;
R⁹ is selected from H, halo, alkyl, alkynyl, and alkoxy; or
R⁸ and R⁹ together with the carbon atoms to which they are attached form a cycloalkyl, heteroaryl, or heterocyclyl; or
R⁹ and R¹⁰ together with the carbon atoms to which they are attached form a cycloalkyl or heterocyclyl;
R¹² is H, deuterium or carboxy;
R¹³ is H, deuterium, halo or thioalkyl;
R¹⁴ is H, sulfonyl, carbonyl or phosphate;
wherein each alkyl, alkenyl, alkynyl, alkoxy, amino, amido, alkylcarbonyl, alkoxycarbonyl, cycloalkyl, heterocyclyl, aryl, and heteroaryl is independently unsubstituted or substituted with one or more substituents selected from deuterium, halo, CN, nitro, hydroxy, alkyl, amino, amido, oxo, boronate, cycloalkyl, aryl, heterocyclyl, heteroaryl, carbonyl, carboxy, acyloxy, alkoxy, thio, phosphino, phosphono, phosphate, sulfinyl, sulfonyl, sulfonamido, ureido, amidino, hydroxyamidino, iminosulfanoyl, 2-tetrahydrothiophene-1,1-dioxide, and isothiazolidine-1,1-dioxide; and
wherein at least one of R⁷, R⁸, R⁹, R¹⁰, and R¹¹ is not H; and provided that when E is CR⁷, G is CR⁸, and one of R⁷, R⁸, R⁹, R¹⁰, and R¹¹ is unsubstituted alkyl, halo, or haloalkyl, then the ring to which R⁷, R⁸, R⁹, R¹⁰, and R¹¹ are attached is disubstituted.

### Variables A, M, Q, T, V and D of Formula I or I′

Below are exemplary embodiments of variables A, M, Q, T, V and D of the disclosed compound of Formula I. The values for the remaining variables are as described above and below.

In some embodiments, A is

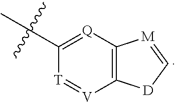

In some embodiments, A is

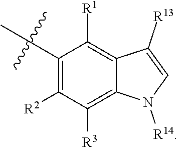

In some embodiments, A is

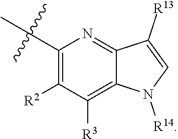

In some embodiments, A is

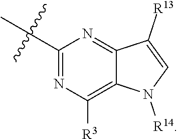

In some embodiments, A is

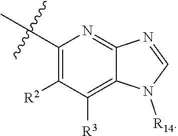

In some embodiments, A is

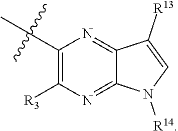

In certain embodiments, A is

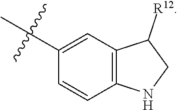

In other embodiments, A is

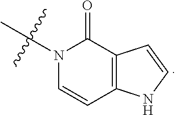 [SMILES: *n1ccc2[nH]ccc2c1=O]

In some embodiments, A is

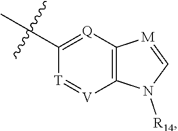

M is CH or C-deuterium; Q is CR¹; T is CR²; V is CR³, and R¹⁴ is H or phosphate.
Variables R¹, R², R³, R^{4a}, and R^{4c} of Formula I or I′

Below are exemplary embodiments of variables R¹, R², R³, R^{4a}, and R^{4c} of the disclosed compound of Formula I. The values for the remaining variables are as described above and below.

In some embodiments, R¹ is selected from H, halo, alkyl, amido, phosphino, sulfonyl and sulfonamide; R² is H or halo; R³ is H or halo; R^{4a} is H or halo; and R^{4c} is H or halo.

In some embodiments, R¹ is H; R² is H; R³ is H; and R^{4c} is F.

In some embodiments, R¹ is methyl; R² is F; R³ is H; and R^{4c} is F.

In some embodiments, R¹ is methyl; R² is F; R³ is H; R^{4a} is F, and R^{4c} is F.

In some embodiments, R¹ is methyl; R² is F; R³ is F; and R^{4c} is F.

In some embodiments, R¹ is CH₂CHF₂; R² is F; R³ is H; and R^{4c} is F.

In some embodiments, R¹ is CH₂CF₃; R² is F; R³ is H; and R^{4c} is F.

In some embodiments, R¹ is F; R² is F; R³ is H; and R^{4c} is F.

In some embodiments, R¹ is F; R² is F; R³ is H; and R^{4c} is H.

In some embodiments, R¹ is F; R² is F; R³ is F; and R^{4c} is F.

In some embodiments, R¹ is F; R² is F; R³ is H; R^{4a} is deuterium, and R^{4c} is H.

In some embodiments, R¹ is —PO(Me)₂; R² is F; R³ is H; and R^{4c} is F.

In some embodiments, R¹ is —PO(Me)₂; R² is F; R³ is H; and R^{4c} is H.

In some embodiments, R¹ is —SO₂Me; R² is F; R³ is H; and R^{4c} is F.

In some embodiments, R¹ is —SO₂Me; R² is F; R³ is H; and R^{4c} is H.

In some embodiments, R¹ is —SO₂Me; R² is F; R³ is F; and R^{4c} is F.

In some embodiments, R¹ is —SONHMe; R² is F; R³ is H; and R^{4c} is F.

In some embodiments, R¹ is H; R² is H; R³ is H; and R^{4c} is F.

In some embodiments, R¹ is —CONHMe; R² is F; R³ is H; and R^{4c} is F.

In some embodiments, R¹ is —CH₂POMe₂; R² is F; R³ is H; and R^{4c} is F.

In some embodiments, R¹ is —CH₂SONHMe; R² is F; R³ is H; and R^{4c} is F.

In some embodiments, R¹ is —CH₂SO₂Me; R² is F; R³ is H; and R^{4c} is F.

In some embodiments, R¹ is —CH₂CH₂SO₂Me; R² is F; R³ is H; and R^{4c} is F.

In some embodiments, R¹ is —CH₂CH₂POMe₂; R² is F; R³ is H; and R^{4c} is F.

In some embodiments, R¹ is

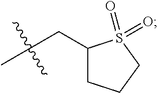 [SMILES: *CC1CCCS1(=O)=O]

In some embodiments, R¹ is

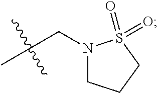 [SMILES: *CN1CCCS1(=O)=O]

R² is F; R³ is H; and R^{4c} is F.

In some embodiments, R¹ is alkyl and is unsubstituted or substituted with one or more substituents selected from halo, sulfonyl, phosphino, 2-tetrahydrothiophene-1,1-dioxide, and isothiazolidine-1,1-dioxide.

### Variables U, W, X, Y, and Z of Formula I or I′

Below are exemplary embodiments of variables U, W, X, Y, and Z of the disclosed compound of Formula I. The values for the remaining variables are as described above and below.

In some embodiments, U is C; W is NH; X is C; Y is selected from CH, C-Me, and C—CMe₂OH; and Z is N.

In some embodiments, U is C; W is NH; X is C; Y is N; and Z is N.

In some embodiments, U is C; W is NH; X is C; Y is N; and Z is CH.

In some embodiments, U is C; W is N; X is C; Y is CH; and Z is S.

In some embodiments, U is C; W is N; X is C; Y is CH; and Z is O.

In some embodiments, U is C; W is N; X is C; Y is CH; and Z is CH or C-Me.

In some embodiments, U is C; W is N; X is N; Y is CH; and Z is CH.

In some embodiments, U is C; W is N; X is N; Y is CH; and Z is NH.

In some embodiments, U is N; W is N; X is C; Y is CH; and Z is CH.

### Variables J, K, and L of Formula I or I′

Below are exemplary embodiments of variable J, K, and L of the disclosed compound of Formula I. The values for the remaining variables are as described above and below.

In some embodiments, J is CR^{4a}; K is CR^{4b}; L is CR^{4c}; and each of R^{4a}, R^{4b}, and R^{4c} is independently selected from H, deuterium, and halo. In certain embodiments, R^{4a} is H; R^{4b} is H; and R^{4c} is F.

Variables R⁵ and R⁶ of Formula I or I′

Below are exemplary embodiments of variables R⁵ and R⁶ of the disclosed compound of Formula I. The values for the remaining variables are as described above and below.

In some embodiments, R⁵ is selected from H, alkyl, amino and hydroxy; R⁶ is H, CN or alkyl; or R⁵ and R⁶ together with the carbon atoms to which they are attached form an cycloalkyl or a heterocyclyl. In certain embodiments, each alkyl, cycloalkyl and heterocyclyl is independently unsubstituted or substituted with one or more substituents selected from deuterium, CN, halo, hydroxy, and alkoxy.

In some embodiments, R⁵ is selected from H, methyl, CD₃, t-butyl, —CH₂CN, —CH₂OCH₃, —(CH₂)₂O—(CH₂)₂O—CHMe₂, —NHMe, and hydroxy; R⁶ is selected from H, methyl, —CH₂OCH₃, —CHF₂, CF₃, and CN; or R⁵ and R⁶ together with the carbon atoms to which they are attached form a

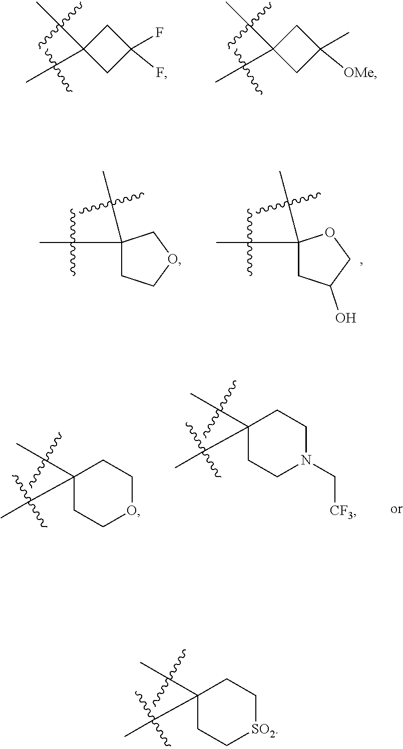

Variables E, G, R⁷, R⁸, R⁹, R¹⁰, and R¹¹ of Formula I or I′

Below are exemplary embodiments of variables E, G, R⁷, R⁸, R⁹, R¹⁰, and R¹¹ of the disclosed compound of Formula I. The values for the remaining variables are as described above.

In some embodiments, E is CR⁷; G is CR⁸; R⁷ is H or halo; R⁸ is alkyl or cycloalkyl, each unsubstituted or substituted with carboxy; R⁹ is H; R¹⁰ is H or halo; and R¹¹ is H.

In some embodiments, R⁷ is H; R⁸ is —CH₂CH₂COOH; R⁹ is H; R¹⁰ is H; and R¹¹ is H.

In some embodiments, R⁷ is F; R⁸ is —CH₂CH₂COOH; R⁹ is H; R¹⁰ is H; and R¹¹ is H.

In some embodiments, R⁷ is F; R⁸ is —CH₂CH₂COOH; R⁹ is H; R¹⁰ is F; and R¹¹ is H.

In some embodiments, R⁷ is F; R⁸ is —CH₂CH₂COOH; R⁹ is H; R¹⁰ is H; and R¹¹ is F

In some embodiments, R⁷ is F; R⁸ is —CH₂CHMeCOOH; R⁹ is H; R¹⁰ is H; and R¹¹ is H.

In some embodiments, R⁷ is H; R⁸ is —CH₂CH₂OH; R⁹ is H; R¹⁰ is F; and R¹¹ is H.

In some embodiments, R⁷ is H; R⁸ is —CH₂CH₂OH; R⁹ is H; R¹⁰ is H; and R¹¹ is H.

In some embodiments, R⁷ is H; R⁸ is —(CH₂)₃PO(OH)₂; R⁹ is H; R¹⁰ is H; and R¹¹ is H.

In some embodiments, E is N; R⁸ is —CH₂CH₂COOH; R⁹ is H; R¹⁰ is H; and R¹¹ is H.

In some embodiments, R⁷ is F; R⁸ is

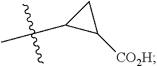 [SMILES: *C1CC1C(=O)O]

R⁹ is H; R¹⁰ is H; and R¹¹ is H.

In some embodiments,
G is CR⁸;
R⁸ is selected from H, halo, alkyl, alkenyl, alkynyl, alkoxy, amido, carboxy, alkoxycarbonyl, thioalkyl, cycloalkyl, and heterocyclyl;
R⁹ is selected from H, halo, alkyl, alkynyl, and alkoxy; wherein
at least one of R⁸ and R⁹ is alkyl, alkenyl, alkynyl, alkoxy or cycloalkyl substituted with carboxy, hydroxy, amido, phosphate, or sulfonamido.

### Compounds of Formula IA

In some embodiments, the compound of Formula I has the structure of compound IA:

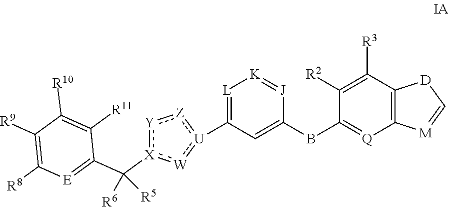

wherein
B is O or C═O;
M is CR¹³;
Q is CR¹ or N;
T is CR²;
V is CR³;
D is NR¹⁴ or S;
J is CR^{4a};
K is CR^{4b} or N;
L is CR^{4c};
E is CR⁷ or N;
U is N or C;
W is N, NR^{a}, or S;
X is N or C;
Y is CR^{b}, N or NR^{c};
Z is N, NR^{d}, O, S, or CH;
[symbol] is a single or double bond;
R^{a}, R^{c}, and R^{d} are each independently selected from H, alkyl, or aralkyl;
R^{b} is selected from H, halo, or alkyl;
R¹ is selected from H, halo, alkyl, alkenyl, amido, cycloalkyl, heterocyclyl, heteroaryl, phosphino, thioalkyl, sulfinyl, and sulfonyl;
R² is selected from H and halo;
R³ is selected from H or halo;
R^{4a}, R^{4b}, and R^{4c} are each independently selected from H, halo, and alkyl;
R⁵ is selected from H, CN, alkyl, hydroxy, alkoxy, and amino;
R⁶ is H or alkyl; or
R⁵ and R⁶ together with the carbon atoms to which they are attached form a vinyl, cycloalkyl or heterocyclyl;
R⁷ and R¹¹ are each independently selected from H and halo; or
R⁸ and R¹⁰ are each independently selected from H, halo, alkyl, alkenyl, thioalkyl, and cycloalkyl; or
R⁷ and R⁸ together with the carbon atoms to which they are attached form a cycloalkyl;
R⁹ is selected from H, halo, alkyl, and alkynyl; or
R⁸ and R⁹ together with the carbon atoms to which they are attached form a cycloalkyl, heteroaryl, and heterocyclyl;
R¹³ is H, deuterium, or halo;
R¹⁴ is H, carbonyl or phosphate;
wherein each alkyl, alkenyl, alkynyl, alkoxy, amino, amido, cycloalkyl, heterocyclyl, and heteroaryl is independently unsubstituted or substituted with one or more substituents selected from deuterium, halo, CN, hydroxy, alkyl, amino, amido, cycloalkyl, heterocyclyl, heteroaryl, carbonyl, carboxy, alkoxy, phosphino, phosphate, sulfonyl, sulfonamido, iminosulfanoyl, and 2-tetrahydrothiophene-1,1-dioxide; and
wherein at least one of R⁷, R⁸, R⁹, R¹⁰, and R¹¹ is not H.

In some embodiments of Formula IA,
B is O or C═O;
M is CR¹³;
Q is CR¹ or N;
T is CR²;
V is CR³;
D is NR¹⁴ or S;
J is CR^{4a};
K is CR^{4b} or N;
L is CR^{4c};
E is CR⁷ or N;
U is N or C;
W is N or NR^{a};
X is N or C;
Y is CR^{b} or N;
Z is N, NR^{d}, O, S, or CH;
[symbol] is a single or double bond;
R^{a} is H;
R^{b}, R^{c}, and R^{d} are each independently H or alkyl;
R¹ is selected from H, halo, alkyl, amido, phosphino, and sulfonyl;
R² is selected from H and halo;
R³ is selected from H or halo;
R^{4a}, R^{4b}, and R^{4c} are each independently selected from H, halo, and alkyl;
R⁵ is selected from H, CN, alkyl, hydroxy, and amino;
R⁶ is H or alkyl; or
R⁵ and R⁶ together with the carbon atoms to which they are attached form a cycloalkyl or a heterocyclyl;
R⁷ and R¹¹ are each independently selected from H and halo; or
R⁹ is selected from H and alkyl;
R¹³ is H or deuterium;
R¹⁴ is H or phosphate;
wherein each alkyl, alkoxy, amino, amido, cycloalkyl, heterocyclyl, and heteroaryl is independently unsubstituted or substituted with one or more substituents selected from deuterium, halo, CN, hydroxy, alkyl, carboxy, alkoxy, phosphino, phosphate, sulfonyl, sulfonamido, iminosulfanoyl, and 2-tetrahydrothiophene-1,1-dioxide; and
wherein at least one of R⁷, R⁸, R⁹, R¹⁰, and R¹¹ is not H.

In some embodiments, the compound of Formula I has the structure of compound IB:

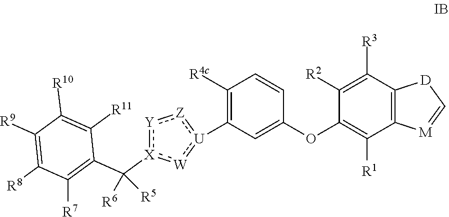

wherein
U is N or C;
W is N or NR^{a}, where R^{a} is H;
X is N or C;
Y is N, C or CR^{b}, where R^{b} is H or alkyl;
Z is N, NR^{d}, O or S, where R^{d} is alkyl;
M is CH or C-deuterium;
D is NR¹⁴;
[symbol] is a single or double bond;
R¹ is H, halo, alkyl, amido, sulfonyl and sulfonamido;
R² is H or halo;
R³ is H or halo;
R⁴, is H or halo;
R⁵ is selected from H, CN, alkyl, hydroxy, and amino;
R⁶ is H, alkyl, or hydroxy; or
R⁵ and R⁶ together with the carbon atoms to which they are attached form a heterocyclyl;
R⁷ is H or halo;
R⁸ is alkyl or cycloalkyl;
R⁹ is H;
R¹⁰ and R¹¹ are each H or halo; and
R¹⁴ is H or phosphate;
wherein each alkyl, amino, alkoxy. alkylcarbonyl, cycloalkyl, and heterocyclyl is independently unsubstituted or substituted with one or more substituents selected from deuterium, halo, CN, hydroxy, alkyl, alkoxy, carboxy, sulfonyl, sulfonamido, phosphino, 2-tetrahydrothiophene-1,1-dioxide, and isothiazolidine-1,1-dioxide; and
wherein at least one of R⁷, R⁸, R⁹, R¹⁰, and R¹¹ is not H.

In some embodiments,
U is C;
W is N or NR^{a}, where R^{a} is H;
X is N or C;
Y is N, C or CR^{b}, where R^{b} is H or alkyl;
Z is N, NR^{d}, or O, where R^{d} is H or alkyl;
M is CH or C-deuterium;
D is NR¹⁴;
R¹ is selected from halo, alkyl, sulfonyl and sulfonamido;
R² is H or halo;
R³ is H or halo;
R^{4c} is halo;
R⁵ is selected from H, CN, and alkyl,
R⁶ is H or alkyl,
R⁷ is H or halo;
R⁸ is alkyl;
R⁹ is H;
R¹⁰ is H or halo;
R¹¹ is H; and
R¹⁴ is H or phosphate;
wherein each alkyl is independently unsubstituted or substituted with one or more substituents selected from deuterium, halo, carboxy, sulfonyl, phosphino, phosphate, iminosulfamoyl, tetrahydrothiophene-1,1-dioxide and isothiazolidine-1,1-dioxide.

In some embodiments,
U is C;
W is N or NR^{a}, where R^{a} is H;
X is N or C;
Y is CR^{b}, where R^{b} is H;
Z is N, or NR^{d}, where R^{d} is H;
M is CH;
R¹ is halo or sulfonyl;
R² is halo;
R³ is H or halo;
R^{4c} is halo;
R⁵ is H, or alkyl,
R⁶ is H,
R⁷ is H or halo;
R⁸ is alkyl substituted with carboxy;
R⁹ is H; and
R¹⁰ is H or halo; and
R¹¹ is H.

### Compounds of Formulas IC and ID

In some embodiments, the compound of Formula I has the structure of compound IC:

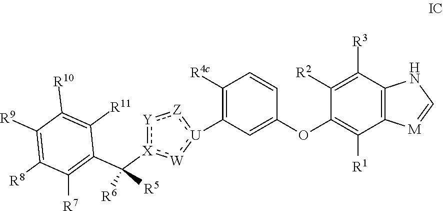

wherein R⁵ is selected from halo, alkyl, alkenyl, alkynyl, hydroxy, alkoxy, alkylcarbonyl, alkoxycarbonyl, amino, and amido; and R⁶ is H or CN.

In some embodiments, the compound of Formula I has the structure of compound ID:

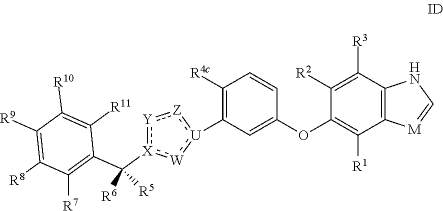

wherein R⁵ is selected from halo, alkyl, alkenyl, alkynyl, hydroxy, alkoxy, alkylcarbonyl, alkoxycarbonyl, amino, and amido; and R⁶ is H or CN.

In some embodiments of compounds of Formulas IC or ID, R⁵ is methyl or hydroxy. The values for the remaining variables are as described above.

### Compound of Formula IE

In some embodiments, the compound of Formula I has the structure of compound IE:

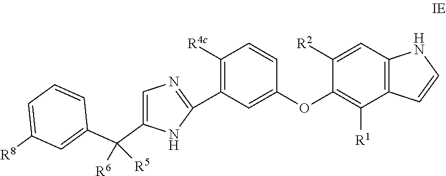

wherein
R¹ is H or F;
R² is H or F;
R^{4c} is H or F;
R⁵ is H, Me or OH;
R⁶ is H or Me; or
R⁵ and R⁶ together with the carbon atoms to which they are attached form a cyclobutyl, cyclopentyl, tetrahydropyran, or dioxylanyl; and
R⁸ is selected from carboxyalkyl, hydroxyalkyl, alkoxycarbonylalkyl, amidoalkyl, and cyclopropyl.

### Compounds of Formula II

Disclosed herein are compounds of Formula II:

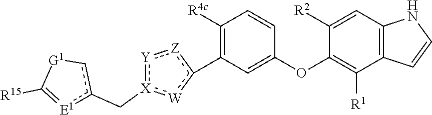

wherein
[symbol] is a single or double bond;
R¹ is alkyl or halo;
R² is halo;
R⁴, is halo;
U is C;
W is N or NH;
X is N or C;
Y is N or CH;
Z is N, NH, or CH;
R¹⁵ is selected from halo, alkyl, alkenyl, alkynyl, alkoxy, amido, carboxy, alkoxycarbonyl, thioalkyl, cycloalkyl, and heterocyclyl;
E¹ is N, or CH; and
G¹ is NH, S or CH₂.

In some embodiments, the compound of Formula II is the compound of Formula IIA:

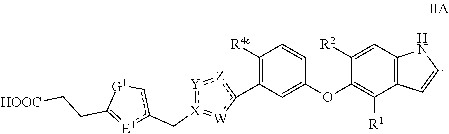

In some embodiments, the compound of Formula II is selected from:

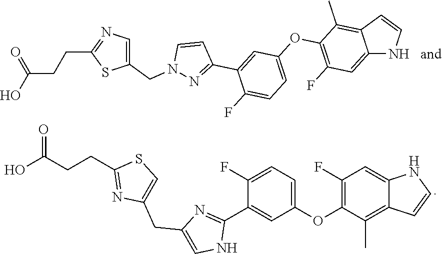

In some embodiments, the compound of Formula I is selected from the following compounds represented in Table 1 below:

TABLE 1

| | |
|---|---|
| 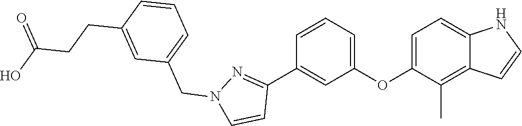 [SMILES: Cc1c(Oc2cccc(-c3ccn(Cc4cccc(CCC(=O)O)c4)n3)c2)ccc2[nH]ccc12] | 1 |
| 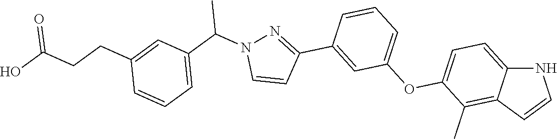 [SMILES: Cc1c(Oc2cccc(-c3ccn(C(C)c4cccc(CCC(=O)O)c4)n3)c2)ccc2[nH]ccc12] | 2 |
| 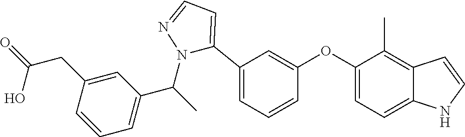 [SMILES: Cc1c(Oc2cccc(-c3ccnn3C(C)c3cccc(CC(=O)O)c3)c2)ccc2[nH]ccc12] | 3 |
| 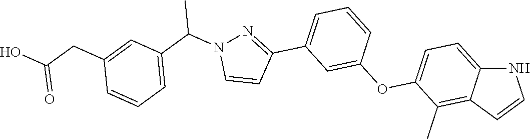 [SMILES: Cc1c(Oc2cccc(-c3ccn(C(C)c4cccc(CC(=O)O)c4)n3)c2)ccc2[nH]ccc12] | 4 |
| 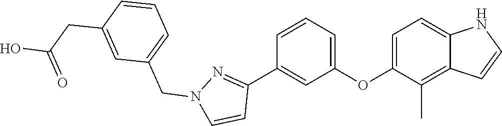 [SMILES: Cc1c(Oc2cccc(-c3ccn(Cc4cccc(CC(=O)O)c4)n3)c2)ccc2[nH]ccc12] | 5 |
| 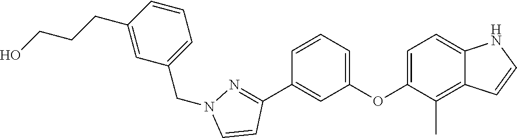 [SMILES: Cc1c(Oc2cccc(-c3ccn(Cc4cccc(CCCO)c4)n3)c2)ccc2[nH]ccc12] | 6 |
| 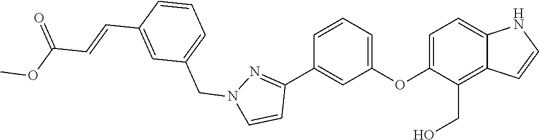 [SMILES: COC(=O)/C=C/c1cccc(Cn2ccc(-c3cccc(Oc4ccc5[nH]ccc5c4CO)c3)n2)c1] | 7 |
| 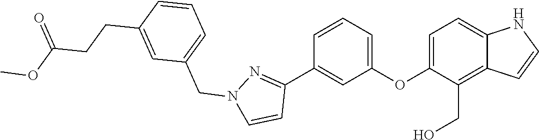 [SMILES: COC(=O)CCc1cccc(Cn2ccc(-c3cccc(Oc4ccc5[nH]ccc5c4CO)c3)n2)c1] | 8 |
| 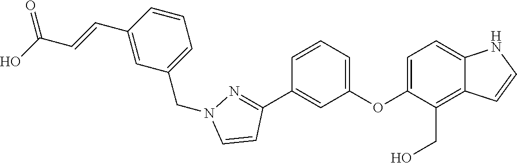 [SMILES: O=C(O)/C=C/c1cccc(Cn2ccc(-c3cccc(Oc4ccc5[nH]ccc5c4CO)c3)n2)c1] | 9 |
| 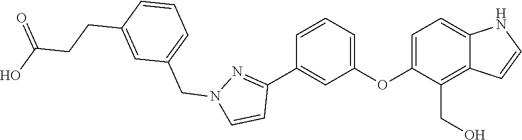 [SMILES: O=C(O)CCc1cccc(Cn2ccc(-c3cccc(Oc4ccc5[nH]ccc5c4CO)c3)n2)c1] | 10 |
| 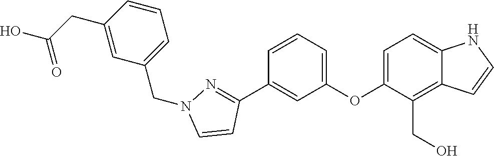 [SMILES: O=C(O)Cc1cccc(Cn2ccc(-c3cccc(Oc4ccc5[nH]ccc5c4CO)c3)n2)c1] | 11 |
| 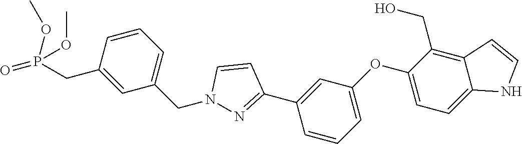 [SMILES: COP(=O)(Cc1cccc(Cn2ccc(-c3cccc(Oc4ccc5[nH]ccc5c4CO)c3)n2)c1)OC] | 12 |
| 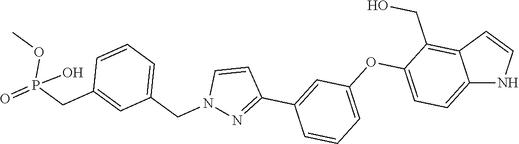 [SMILES: COP(=O)(O)Cc1cccc(Cn2ccc(-c3cccc(Oc4ccc5[nH]ccc5c4CO)c3)n2)c1] | 13 |
| 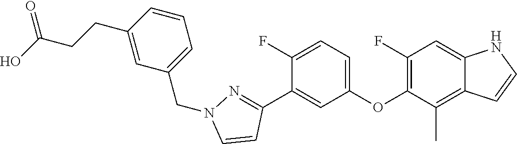 [SMILES: Cc1c(Oc2ccc(F)c(-c3ccn(Cc4cccc(CCC(=O)O)c4)n3)c2)c(F)cc2[nH]ccc12] | 14 |
| 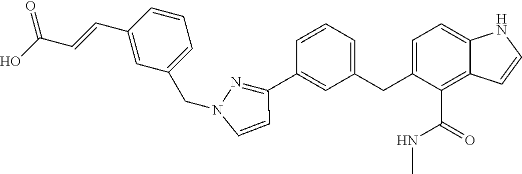 [SMILES: CNC(=O)c1c(Cc2cccc(-c3ccn(Cc4cccc(/C=C/C(=O)O)c4)n3)c2)ccc2[nH]ccc12] | 15 |
| 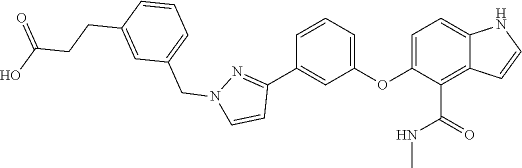 [SMILES: CNC(=O)c1c(Oc2cccc(-c3ccn(Cc4cccc(CCC(=O)O)c4)n3)c2)ccc2[nH]ccc12] | 16 |
| 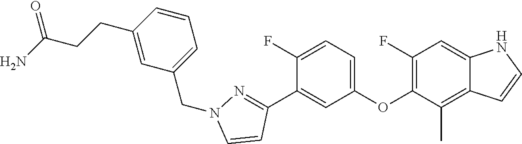 [SMILES: Cc1c(Oc2ccc(F)c(-c3ccn(Cc4cccc(CCC(N)=O)c4)n3)c2)c(F)cc2[nH]ccc12] | 17 |
| 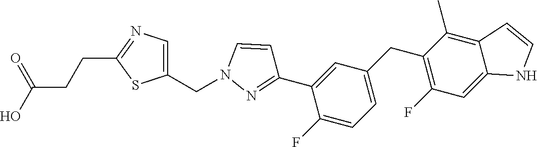 [SMILES: Cc1c(Cc2ccc(F)c(-c3ccn(Cc4cnc(CCC(=O)O)s4)n3)c2)c(F)cc2[nH]ccc12] | 18 |
| 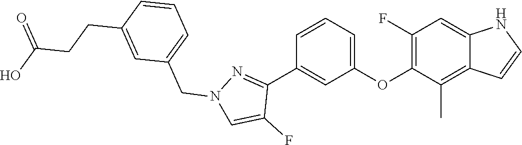 [SMILES: Cc1c(Oc2cccc(-c3nn(Cc4cccc(CCC(=O)O)c4)cc3F)c2)c(F)cc2[nH]ccc12] | 19 |
| 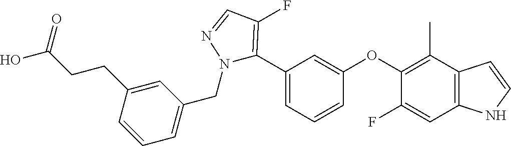 [SMILES: Cc1c(Oc2cccc(-c3c(F)cnn3Cc3cccc(CCC(=O)O)c3)c2)c(F)cc2[nH]ccc12] | 20 |
| 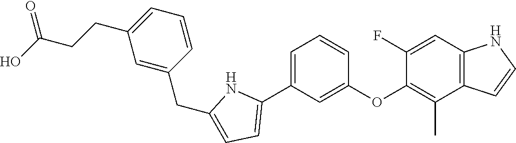 [SMILES: Cc1c(Oc2cccc(-c3ccc(Cc4cccc(CCC(=O)O)c4)[nH]3)c2)c(F)cc2[nH]ccc12] | 21 |
| 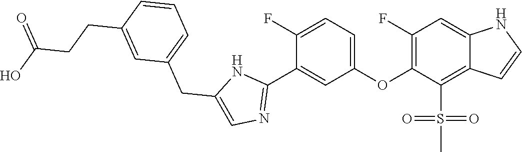 [SMILES: CS(=O)(=O)c1c(Oc2ccc(F)c(-c3ncc(Cc4cccc(CCC(=O)O)c4)[nH]3)c2)c(F)cc2[nH]ccc12] | 22 |
| 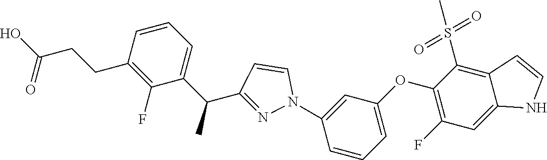 [SMILES: C[C@H](c1ccn(-c2cccc(Oc3c(F)cc4[nH]ccc4c3S(C)(=O)=O)c2)n1)c1cccc(CCC(=O)O)c1F] | 23A |
| 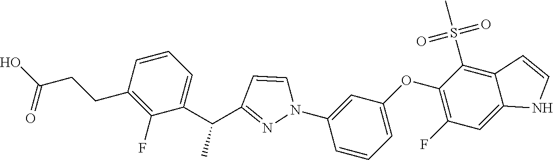 [SMILES: C[C@@H](c1ccn(-c2cccc(Oc3c(F)cc4[nH]ccc4c3S(C)(=O)=O)c2)n1)c1cccc(CCC(=O)O)c1F] | 23B |
| 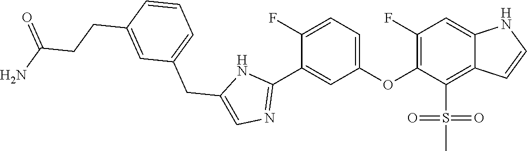 [SMILES: CS(=O)(=O)c1c(Oc2ccc(F)c(-c3ncc(Cc4cccc(CCC(N)=O)c4)[nH]3)c2)c(F)cc2[nH]ccc12] | 24 |
| 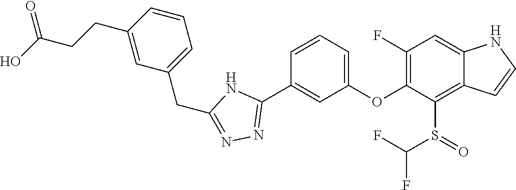 [SMILES: O=C(O)CCc1cccc(Cc2nnc(-c3cccc(Oc4c(F)cc5[nH]ccc5c4S(=O)C(F)F)c3)[nH]2)c1] | 25 |
| 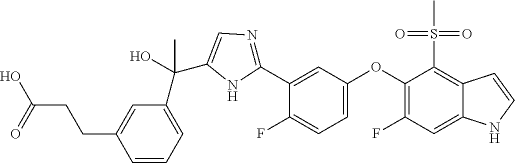 [SMILES: CC(O)(c1cccc(CCC(=O)O)c1)c1cnc(-c2cc(Oc3c(F)cc4[nH]ccc4c3S(C)(=O)=O)ccc2F)[nH]1] | 26 |
| 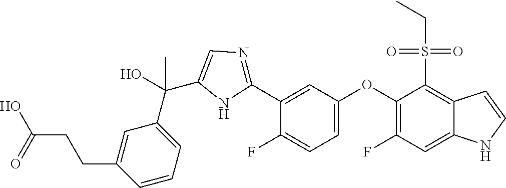 [SMILES: CCS(=O)(=O)c1c(Oc2ccc(F)c(-c3ncc(C(C)(O)c4cccc(CCC(=O)O)c4)[nH]3)c2)c(F)cc2[nH]ccc12] | 27 |
| 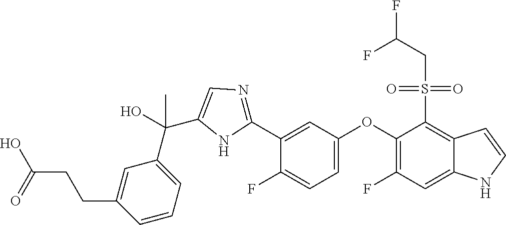 [SMILES: CC(O)(c1cccc(CCC(=O)O)c1)c1cnc(-c2cc(Oc3c(F)cc4[nH]ccc4c3S(=O)(=O)CC(F)F)ccc2F)[nH]1] | 28 |
| 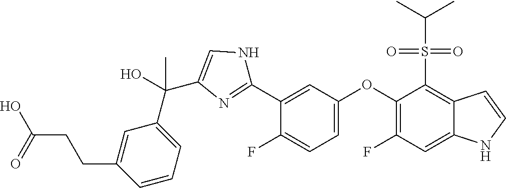 [SMILES: CC(C)S(=O)(=O)c1c(Oc2ccc(F)c(-c3nc(C(C)(O)c4cccc(CCC(=O)O)c4)c[nH]3)c2)c(F)cc2[nH]ccc12] | 29 |
| 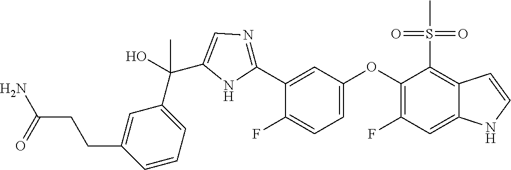 [SMILES: CC(O)(c1cccc(CCC(N)=O)c1)c1cnc(-c2cc(Oc3c(F)cc4[nH]ccc4c3S(C)(=O)=O)ccc2F)[nH]1] | 30 |
| 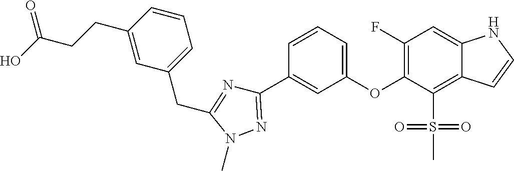 [SMILES: Cn1nc(-c2cccc(Oc3c(F)cc4[nH]ccc4c3S(C)(=O)=O)c2)nc1Cc1cccc(CCC(=O)O)c1] | 31 |
| 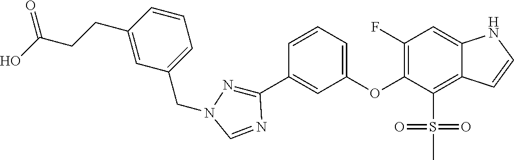 [SMILES: CS(=O)(=O)c1c(Oc2cccc(-c3ncn(Cc4cccc(CCC(=O)O)c4)n3)c2)c(F)cc2[nH]ccc12] | 32 |
| 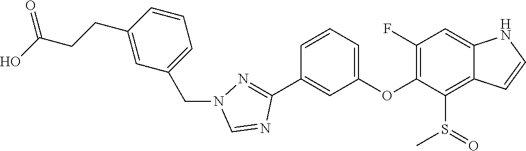 [SMILES: CS(=O)c1c(Oc2cccc(-c3ncn(Cc4cccc(CCC(=O)O)c4)n3)c2)c(F)cc2[nH]ccc12] | 33 |
| 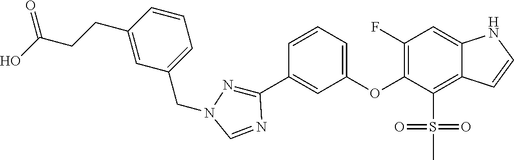 [SMILES: CS(=O)(=O)c1c(Oc2cccc(-c3ncn(Cc4cccc(CCC(=O)O)c4)n3)c2)c(F)cc2[nH]ccc12] | 34 |
| 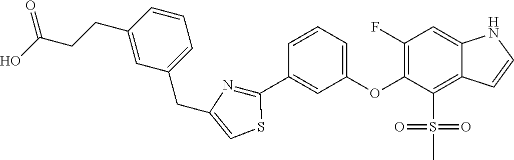 [SMILES: CS(=O)(=O)c1c(Oc2cccc(-c3nc(Cc4cccc(CCC(=O)O)c4)cs3)c2)c(F)cc2[nH]ccc12] | 35 |
| 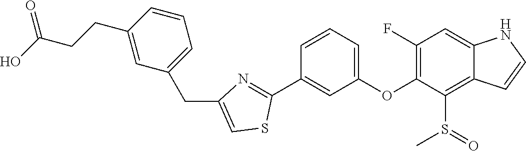 [SMILES: CS(=O)c1c(Oc2cccc(-c3nc(Cc4cccc(CCC(=O)O)c4)cs3)c2)c(F)cc2[nH]ccc12] | 36 |
| 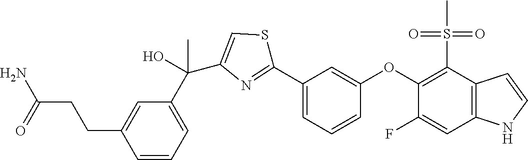 [SMILES: CC(O)(c1cccc(CCC(N)=O)c1)c1csc(-c2cccc(Oc3c(F)cc4[nH]ccc4c3S(C)(=O)=O)c2)n1] | 37 |
| 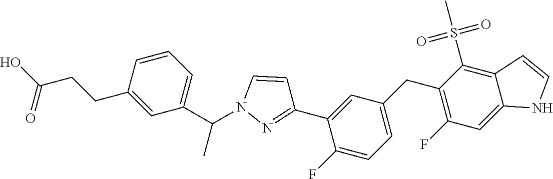 [SMILES: CC(c1cccc(CCC(=O)O)c1)n1ccc(-c2cc(Cc3c(F)cc4[nH]ccc4c3S(C)(=O)=O)ccc2F)n1] | 38 |
| 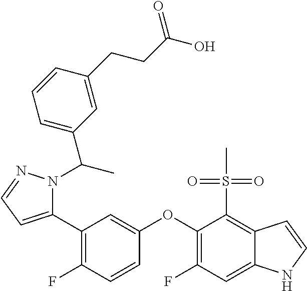 [SMILES: CC(c1cccc(CCC(=O)O)c1)n1nccc1-c1cc(Oc2c(F)cc3[nH]ccc3c2S(C)(=O)=O)ccc1F] | 39 |
| 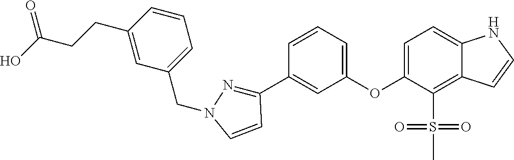 [SMILES: CS(=O)(=O)c1c(Oc2cccc(-c3ccn(Cc4cccc(CCC(=O)O)c4)n3)c2)ccc2[nH]ccc12] | 40 |
| 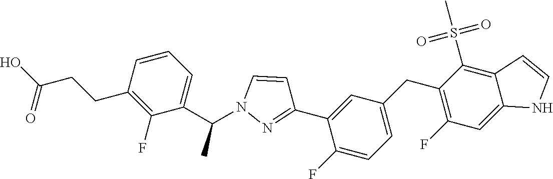 [SMILES: C[C@@H](c1cccc(CCC(=O)O)c1F)n1ccc(-c2cc(Cc3c(F)cc4[nH]ccc4c3S(C)(=O)=O)ccc2F)n1] | 41A |
| 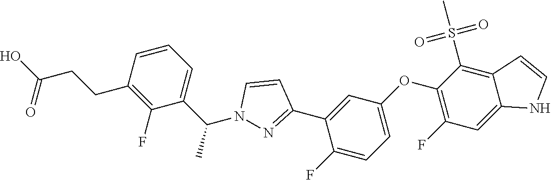 [SMILES: C[C@H](c1cccc(CCC(=O)O)c1F)n1ccc(-c2cc(Oc3c(F)cc4[nH]ccc4c3S(C)(=O)=O)ccc2F)n1] | 41B |
| 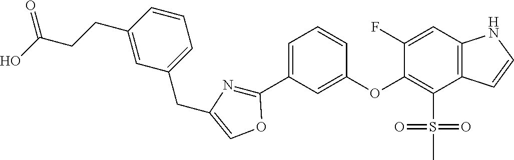 [SMILES: CS(=O)(=O)c1c(Oc2cccc(-c3nc(Cc4cccc(CCC(=O)O)c4)co3)c2)c(F)cc2[nH]ccc12] | 42 |
| 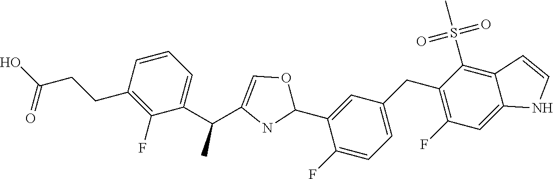 [SMILES: C[C@H](C1=COC(c2cc(Cc3c(F)cc4[nH]ccc4c3S(C)(=O)=O)ccc2F)N1)c1cccc(CCC(=O)O)c1F] | 43A |
| 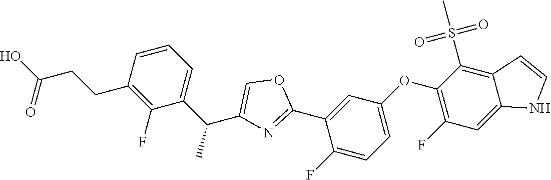 [SMILES: C[C@@H](c1coc(-c2cc(Oc3c(F)cc4[nH]ccc4c3S(C)(=O)=O)ccc2F)n1)c1cccc(CCC(=O)O)c1F] | 43B |
| 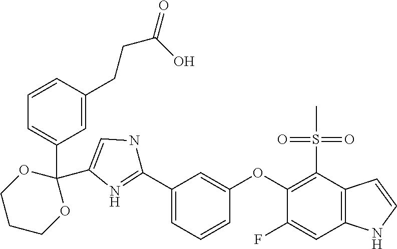 [SMILES: CS(=O)(=O)c1c(Oc2cccc(-c3nc(C4(c5cccc(CCC(=O)O)c5)OCCCO4)c[nH]3)c2)c(F)cc2[nH]ccc12] | 44 |
| 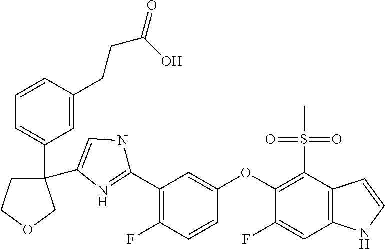 [SMILES: CS(=O)(=O)c1c(Oc2ccc(F)c(-c3nc(C4(c5cccc(CCC(=O)O)c5)CCOC4)c[nH]3)c2)c(F)cc2[nH]ccc12] | 45 |
| 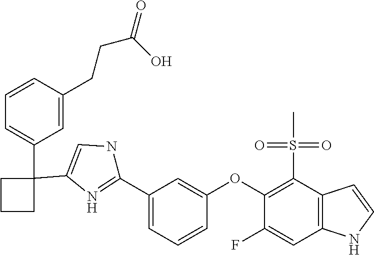 [SMILES: CS(=O)(=O)c1c(Oc2cccc(-c3nc(C4(c5cccc(CCC(=O)O)c5)CCC4)c[nH]3)c2)c(F)cc2[nH]ccc12] | 46 |
| 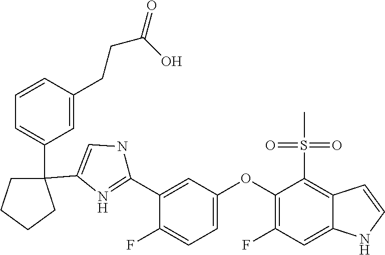 [SMILES: CS(=O)(=O)c1c(Oc2ccc(F)c(-c3nc(C4(c5cccc(CCC(=O)O)c5)CCCC4)c[nH]3)c2)c(F)cc2[nH]ccc12] | 47 |
| 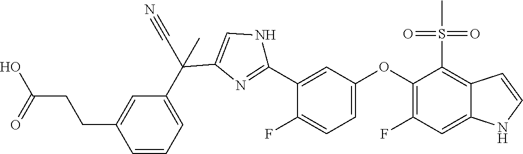 [SMILES: CC(C#N)(c1cccc(CCC(=O)O)c1)c1c[nH]c(-c2cc(Oc3c(F)cc4[nH]ccc4c3S(C)(=O)=O)ccc2F)n1] | 48 |
| 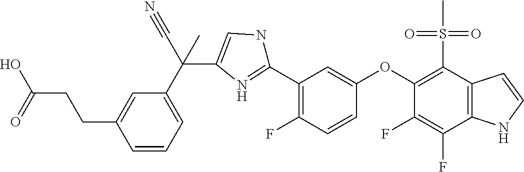 [SMILES: CC(C#N)(c1cccc(CCC(=O)O)c1)c1c[nH]c(-c2cc(Oc3c(F)c(F)c4[nH]ccc4c3S(C)(=O)=O)ccc2F)n1] | 49 |
| 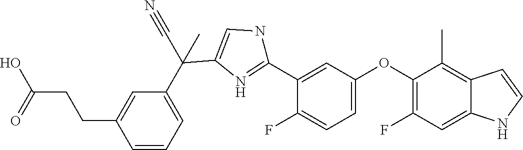 [SMILES: Cc1c(Oc2ccc(F)c(-c3nc(C(C)(C#N)c4cccc(CCC(=O)O)c4)c[nH]3)c2)c(F)cc2[nH]ccc12] | 50 |
| 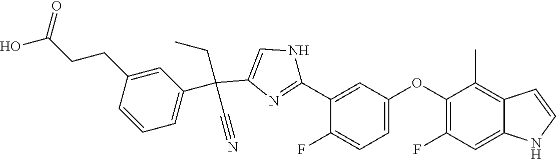 [SMILES: CCC(C#N)(c1cccc(CCC(=O)O)c1)c1c[nH]c(-c2cc(Oc3c(F)cc4[nH]ccc4c3C)ccc2F)n1] | 51 |
| 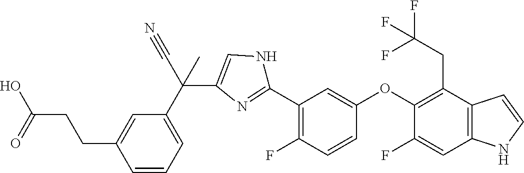 [SMILES: CC(C#N)(c1cccc(CCC(=O)O)c1)c1c[nH]c(-c2cc(Oc3c(F)cc4[nH]ccc4c3CC(F)(F)F)ccc2F)n1] | 52 |
| 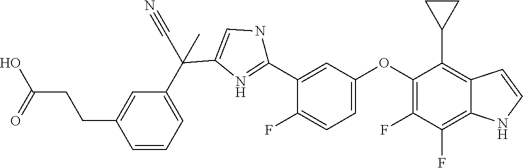 [SMILES: CC(C#N)(c1cccc(CCC(=O)O)c1)c1c[nH]c(-c2cc(Oc3c(F)c(F)c4[nH]ccc4c3C3CC3)ccc2F)n1] | 53 |
| 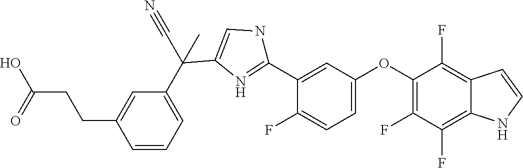 [SMILES: CC(C#N)(c1cccc(CCC(=O)O)c1)c1c[nH]c(-c2cc(Oc3c(F)c(F)c4[nH]ccc4c3F)ccc2F)n1] | 54 |
| 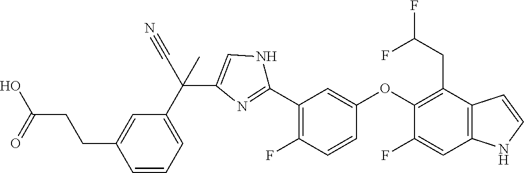 [SMILES: CC(C#N)(c1cccc(CCC(=O)O)c1)c1c[nH]c(-c2cc(Oc3c(F)cc4[nH]ccc4c3CC(F)F)ccc2F)n1] | 55 |
| 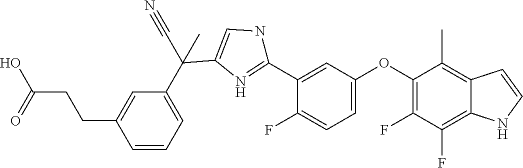 [SMILES: Cc1c(Oc2ccc(F)c(-c3nc(C(C)(C#N)c4cccc(CCC(=O)O)c4)c[nH]3)c2)c(F)c(F)c2[nH]ccc12] | 56 |
| 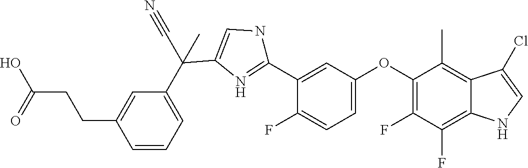 [SMILES: Cc1c(Oc2ccc(F)c(-c3nc(C(C)(C#N)c4cccc(CCC(=O)O)c4)c[nH]3)c2)c(F)c(F)c2[nH]cc(Cl)c12] | 57 |
| 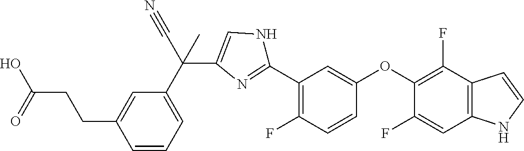 [SMILES: CC(C#N)(c1cccc(CCC(=O)O)c1)c1c[nH]c(-c2cc(Oc3c(F)cc4[nH]ccc4c3F)ccc2F)n1] | 58 |
| 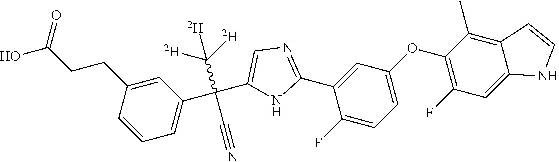 [SMILES: [2H]C([2H])([2H])C(C#N)(c1cccc(CCC(=O)O)c1)c1cnc(-c2cc(Oc3c(F)cc4[nH]ccc4c3C)ccc2F)[nH]1] | 59 |
| 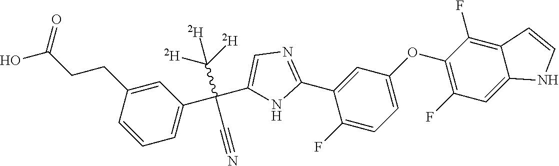 [SMILES: [2H]C([2H])([2H])C(C#N)(c1cccc(CCC(=O)O)c1)c1cnc(-c2cc(Oc3c(F)cc4[nH]ccc4c3F)ccc2F)[nH]1] | 60 |
| 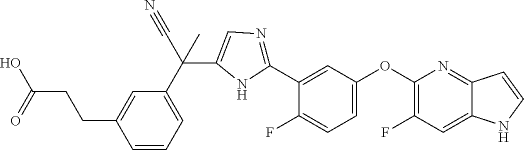 [SMILES: CC(C#N)(c1cccc(CCC(=O)O)c1)c1cnc(-c2cc(Oc3nc4cc[nH]c4cc3F)ccc2F)[nH]1] | 61 |
| 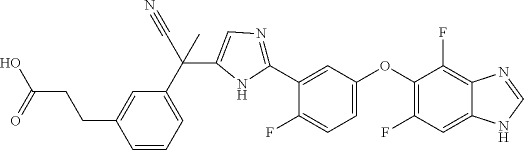 [SMILES: CC(C#N)(c1cccc(CCC(=O)O)c1)c1cnc(-c2cc(Oc3c(F)cc4[nH]cnc4c3F)ccc2F)[nH]1] | 62 |
| 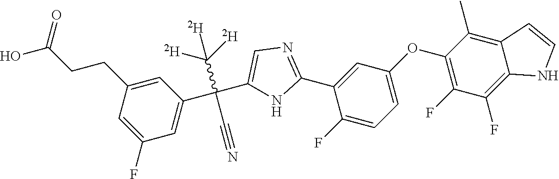 [SMILES: [2H]C([2H])([2H])C(C#N)(c1cc(F)cc(CCC(=O)O)c1)c1cnc(-c2cc(Oc3c(F)c(F)c4[nH]ccc4c3C)ccc2F)[nH]1] | 63 |
| 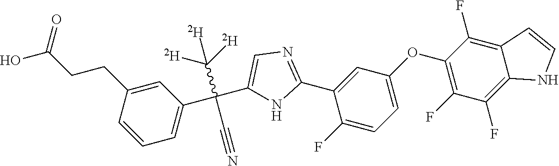 [SMILES: [2H]C([2H])([2H])C(C#N)(c1cccc(CCC(=O)O)c1)c1cnc(-c2cc(Oc3c(F)c(F)c4[nH]ccc4c3F)ccc2F)[nH]1] | 64A |
| 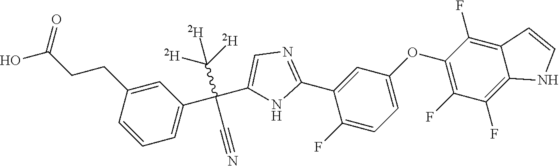 [SMILES: [2H]C([2H])([2H])C(C#N)(c1cccc(CCC(=O)O)c1)c1cnc(-c2cc(Oc3c(F)c(F)c4[nH]ccc4c3F)ccc2F)[nH]1] | 64B |
| 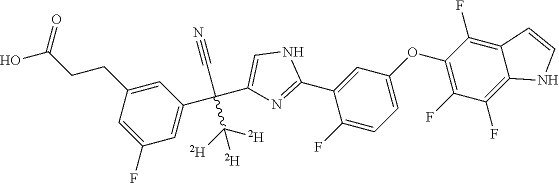 [SMILES: [2H]C([2H])([2H])C(C#N)(c1cc(F)cc(CCC(=O)O)c1)c1c[nH]c(-c2cc(Oc3c(F)c(F)c4[nH]ccc4c3F)ccc2F)n1] | 65 |
| 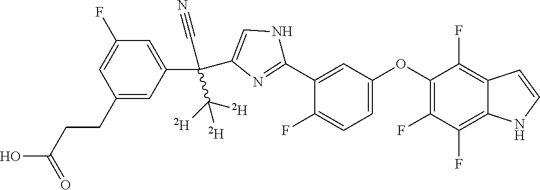 [SMILES: [2H]C([2H])([2H])C(C#N)(c1cc(F)cc(CCC(=O)O)c1)c1c[nH]c(-c2cc(Oc3c(F)c(F)c4[nH]ccc4c3F)ccc2F)n1] | 66 |
| 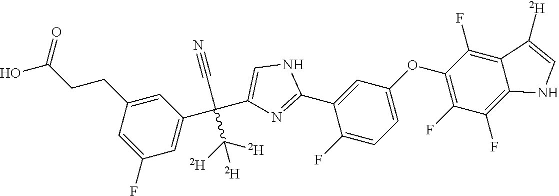 [SMILES: [2H]c1c[nH]c2c(F)c(F)c(Oc3ccc(F)c(-c4nc(C(C#N)(c5cc(F)cc(CCC(=O)O)c5)C([2H])([2H])[2H])c[nH]4)c3)c(F)c12] | 67A |
| 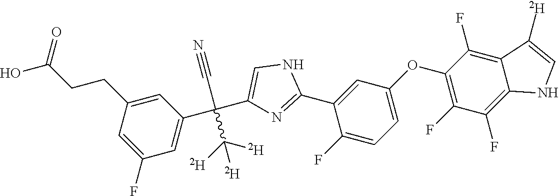 [SMILES: [2H]c1c[nH]c2c(F)c(F)c(Oc3ccc(F)c(-c4nc(C(C#N)(c5cc(F)cc(CCC(=O)O)c5)C([2H])([2H])[2H])c[nH]4)c3)c(F)c12] | 67B |
| 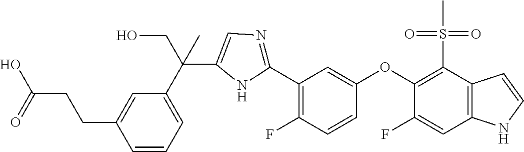 [SMILES: CC(CO)(c1cccc(CCC(=O)O)c1)c1cnc(-c2cc(Oc3c(F)cc4[nH]ccc4c3S(C)(=O)=O)ccc2F)[nH]1] | 68 |
| 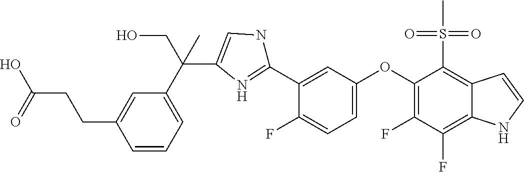 [SMILES: CC(CO)(c1cccc(CCC(=O)O)c1)c1c[nH]c(-c2cc(Oc3c(F)c(F)c4[nH]ccc4c3S(C)(=O)=O)ccc2F)n1] | 69 |
| 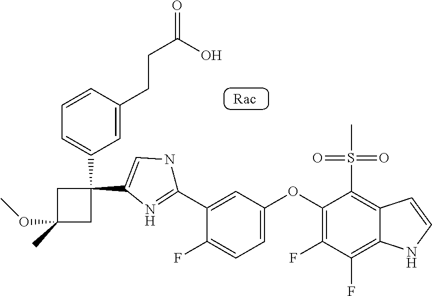 [SMILES: CO[C@]1(C)C[C@@](c2cccc(CCC(=O)O)c2)(c2c[nH]c(-c3cc(Oc4c(F)c(F)c5[nH]ccc5c4S(C)(=O)=O)ccc3F)n2)C1] | 70 |
| 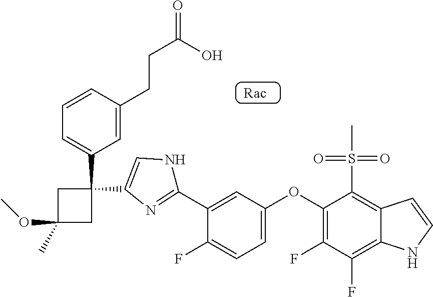 [SMILES: CO[C@]1(C)C[C@@](c2cccc(CCC(=O)O)c2)(c2c[nH]c(-c3cc(Oc4c(F)c(F)c5[nH]ccc5c4S(C)(=O)=O)ccc3F)n2)C1] | 71 |
| 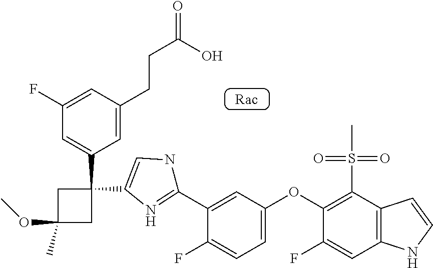 [SMILES: CO[C@]1(C)C[C@@](c2cc(F)cc(CCC(=O)O)c2)(c2c[nH]c(-c3cc(Oc4c(F)cc5[nH]ccc5c4S(C)(=O)=O)ccc3F)n2)C1] | 72 |
| 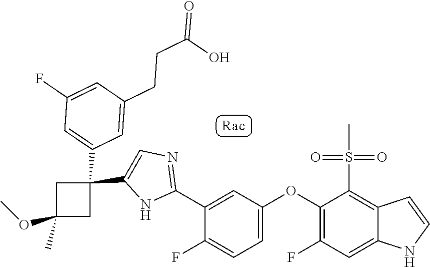 [SMILES: CO[C@]1(C)C[C@](c2cc(F)cc(CCC(=O)O)c2)(c2cnc(-c3cc(Oc4c(F)cc5[nH]ccc5c4S(C)(=O)=O)ccc3F)[nH]2)C1] | 73 |
| 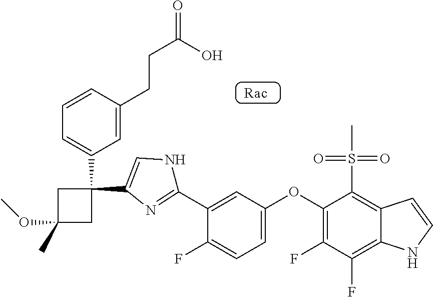 [SMILES: CO[C@]1(C)C[C@@](c2cccc(CCC(=O)O)c2)(c2c[nH]c(-c3cc(Oc4c(F)c(F)c5[nH]ccc5c4S(C)(=O)=O)ccc3F)n2)C1] | 74 |
| 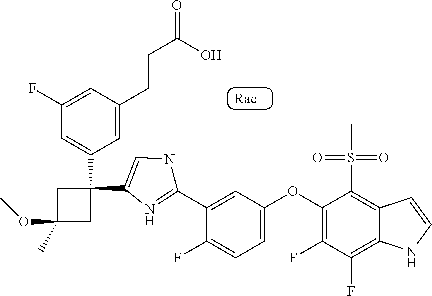 [SMILES: CO[C@]1(C)C[C@](c2cc(F)cc(CCC(=O)O)c2)(c2c[nH]c(-c3cc(Oc4c(F)c(F)c5[nH]ccc5c4S(C)(=O)=O)ccc3F)n2)C1] | 75 |
| 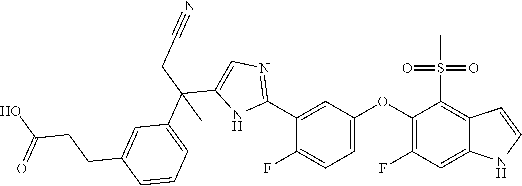 [SMILES: CC(CC#N)(c1cccc(CCC(=O)O)c1)c1cnc(-c2cc(Oc3c(F)cc4[nH]ccc4c3S(C)(=O)=O)ccc2F)[nH]1] | 76 |
| 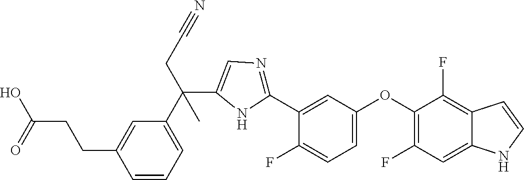 [SMILES: CC(CC#N)(c1cccc(CCC(=O)O)c1)c1cnc(-c2cc(Oc3c(F)cc4[nH]ccc4c3F)ccc2F)[nH]1] | 77 |
| 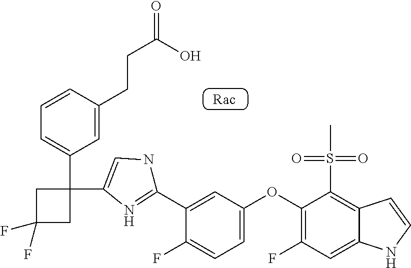 [SMILES: CS(=O)(=O)c1c(Oc2ccc(F)c(-c3nc(C4(c5cccc(CCC(=O)O)c5)CC(F)(F)C4)c[nH]3)c2)c(F)cc2[nH]ccc12] | 78 |
| 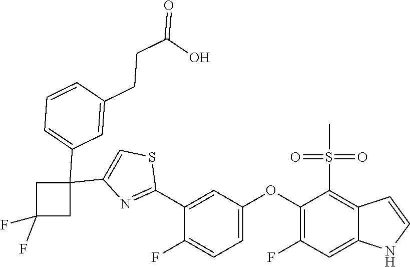 [SMILES: CS(=O)(=O)c1c(Oc2ccc(F)c(-c3nc(C4(c5cccc(CCC(=O)O)c5)CC(F)(F)C4)cs3)c2)c(F)cc2[nH]ccc12] | 79 |
| 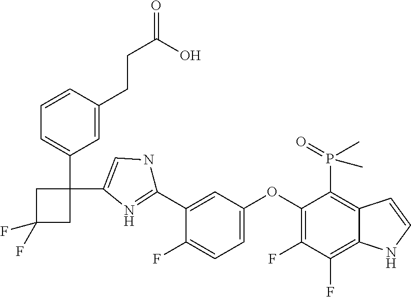 [SMILES: CP(C)(=O)c1c(Oc2ccc(F)c(-c3nc(C4(c5cccc(CCC(=O)O)c5)CC(F)(F)C4)c[nH]3)c2)c(F)c(F)c2[nH]ccc12] | 80 |
| 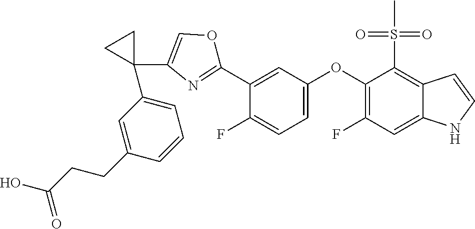 [SMILES: CS(=O)(=O)c1c(Oc2ccc(F)c(-c3nc(C4(c5cccc(CCC(=O)O)c5)CC4)co3)c2)c(F)cc2[nH]ccc12] | 81 |
| 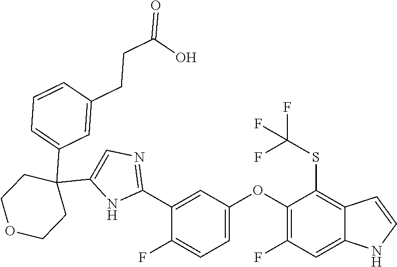 [SMILES: O=C(O)CCc1cccc(C2(c3cnc(-c4cc(Oc5c(F)cc6[nH]ccc6c5SC(F)(F)F)ccc4F)[nH]3)CCOCC2)c1] | 82 |
| 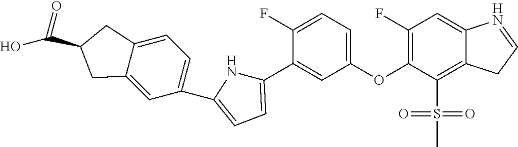 | 83A |
| 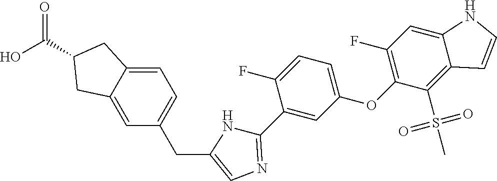 [SMILES: CS(=O)(=O)c1c(Oc2ccc(F)c(-c3ncc(Cc4ccc5c(c4)C[C@H](C(=O)O)C5)[nH]3)c2)c(F)cc2[nH]ccc12] | 83B |
| 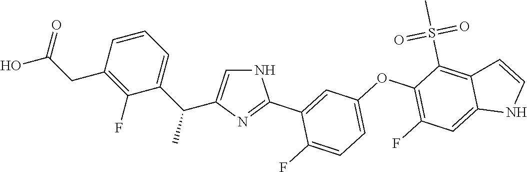 [SMILES: C[C@@H](c1c[nH]c(-c2cc(Oc3c(F)cc4[nH]ccc4c3S(C)(=O)=O)ccc2F)n1)c1cccc(CC(=O)O)c1F] | 84 |
| 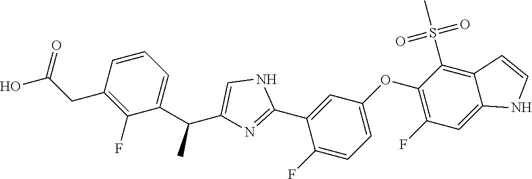 [SMILES: C[C@H](c1c[nH]c(-c2cc(Oc3c(F)cc4[nH]ccc4c3S(C)(=O)=O)ccc2F)n1)c1cccc(CC(=O)O)c1F] | 85 |
| 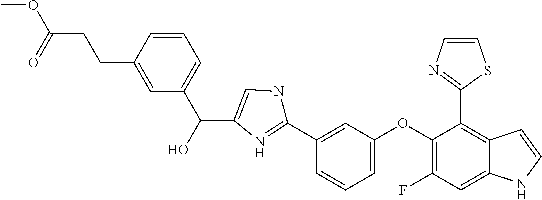 [SMILES: COC(=O)CCc1cccc(C(O)c2c[nH]c(-c3cccc(Oc4c(F)cc5[nH]ccc5c4-c4nccs4)c3)n2)c1] | 86 |
| 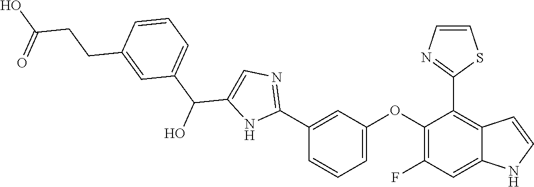 [SMILES: O=C(O)CCc1cccc(C(O)c2cnc(-c3cccc(Oc4c(F)cc5[nH]ccc5c4-c4nccs4)c3)[nH]2)c1] | 87 |
| 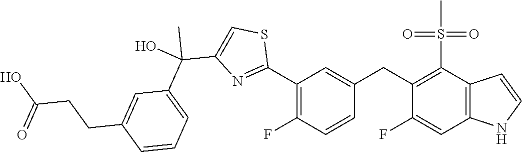 [SMILES: CC(O)(c1cccc(CCC(=O)O)c1)c1csc(-c2cc(Cc3c(F)cc4[nH]ccc4c3S(C)(=O)=O)ccc2F)n1] | 88 |
| 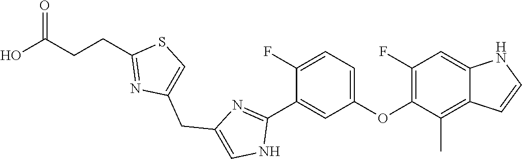 [SMILES: Cc1c(Oc2ccc(F)c(-c3nc(Cc4csc(CCC(=O)O)n4)c[nH]3)c2)c(F)cc2[nH]ccc12] | 89 |
| 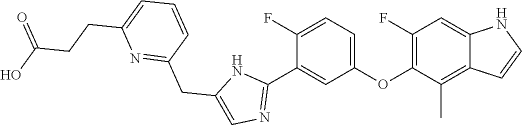 [SMILES: Cc1c(Oc2ccc(F)c(-c3ncc(Cc4cccc(CCC(=O)O)n4)[nH]3)c2)c(F)cc2[nH]ccc12] | 90 |
| 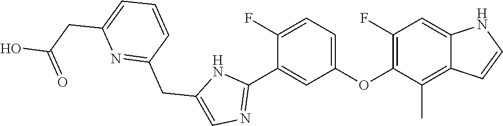 [SMILES: Cc1c(Oc2ccc(F)c(-c3ncc(Cc4cccc(CC(=O)O)n4)[nH]3)c2)c(F)cc2[nH]ccc12] | 91 |
| 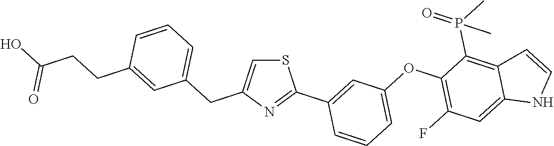 [SMILES: CP(C)(=O)c1c(Oc2cccc(-c3nc(Cc4cccc(CCC(=O)O)c4)cs3)c2)c(F)cc2[nH]ccc12] | 92 |
| 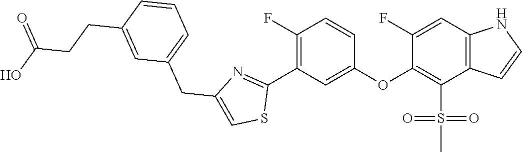 [SMILES: CS(=O)(=O)c1c(Oc2ccc(F)c(-c3nc(Cc4cccc(CCC(=O)O)c4)cs3)c2)c(F)cc2[nH]ccc12] | 93 |
| 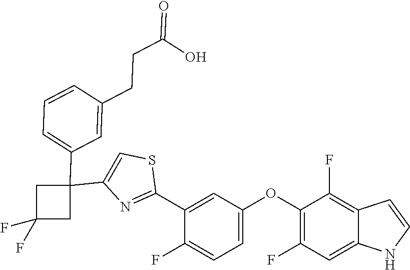 [SMILES: O=C(O)CCc1cccc(C2(c3csc(-c4cc(Oc5c(F)cc6[nH]ccc6c5F)ccc4F)n3)CC(F)(F)C2)c1] | 94 |
| 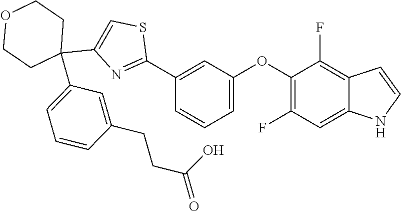 [SMILES: O=C(O)CCc1cccc(C2(c3csc(-c4cccc(Oc5c(F)cc6[nH]ccc6c5F)c4)n3)CCOCC2)c1] | 95 |
| 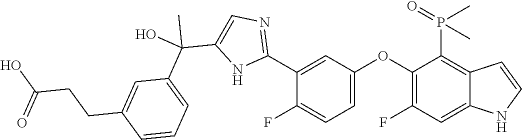 [SMILES: CC(O)(c1cccc(CCC(=O)O)c1)c1cnc(-c2cc(Oc3c(F)cc4[nH]ccc4c3P(C)(C)=O)ccc2F)[nH]1] | 96 |
| 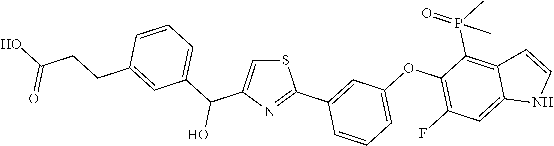 [SMILES: CP(C)(=O)c1c(Oc2cccc(-c3nc(C(O)c4cccc(CCC(=O)O)c4)cs3)c2)c(F)cc2[nH]ccc12] | 97 |
| 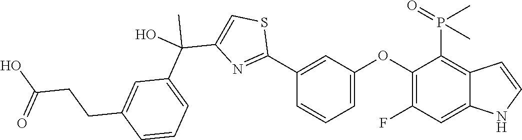 [SMILES: CC(O)(c1cccc(CCC(=O)O)c1)c1csc(-c2cccc(Oc3c(F)cc4[nH]ccc4c3P(C)(C)=O)c2)n1] | 98 |
| 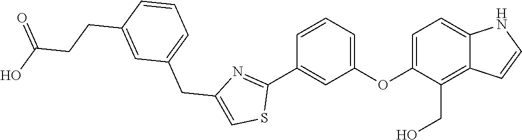 [SMILES: O=C(O)CCc1cccc(Cc2csc(-c3cccc(Oc4ccc5[nH]ccc5c4CO)c3)n2)c1] | 99 |
| 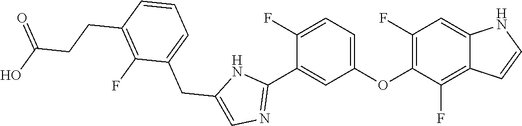 [SMILES: O=C(O)CCc1cccc(Cc2cnc(-c3cc(Oc4c(F)cc5[nH]ccc5c4F)ccc3F)[nH]2)c1F] | 100 |
| 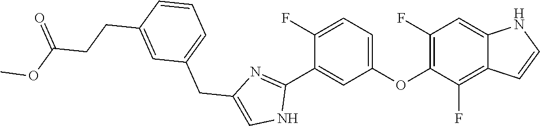 [SMILES: COC(=O)CCc1cccc(Cc2c[nH]c(-c3cc(Oc4c(F)cc5[nH]ccc5c4F)ccc3F)n2)c1] | 101 |
| 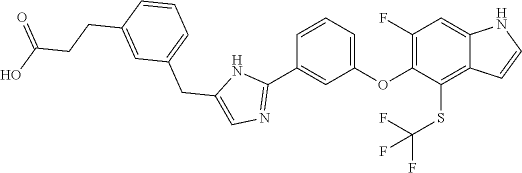 [SMILES: O=C(O)CCc1cccc(Cc2cnc(-c3cccc(Oc4c(F)cc5[nH]ccc5c4SC(F)(F)F)c3)[nH]2)c1] | 102 |
| 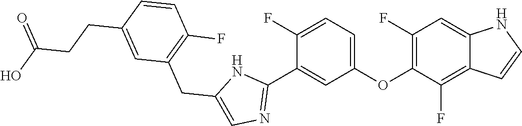 [SMILES: O=C(O)CCc1ccc(F)c(Cc2cnc(-c3cc(Oc4c(F)cc5[nH]ccc5c4F)ccc3F)[nH]2)c1] | 103 |
| 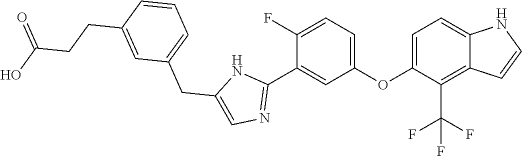 [SMILES: O=C(O)CCc1cccc(Cc2cnc(-c3cc(Oc4ccc5[nH]ccc5c4C(F)(F)F)ccc3F)[nH]2)c1] | 104 |
| 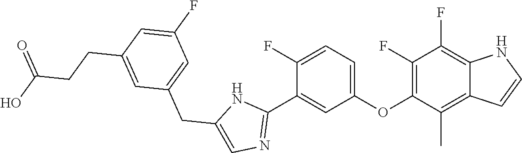 [SMILES: Cc1c(Oc2ccc(F)c(-c3ncc(Cc4cc(F)cc(CCC(=O)O)c4)[nH]3)c2)c(F)c(F)c2[nH]ccc12] | 105 |
| 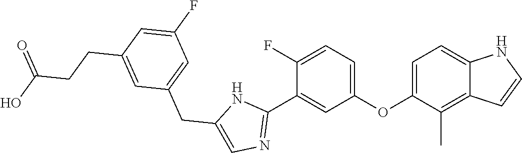 [SMILES: Cc1c(Oc2ccc(F)c(-c3ncc(Cc4cc(F)cc(CCC(=O)O)c4)[nH]3)c2)ccc2[nH]ccc12] | 106 |
| 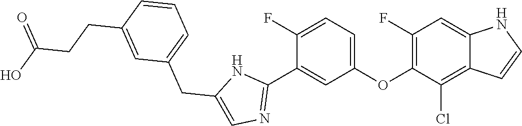 [SMILES: O=C(O)CCc1cccc(Cc2cnc(-c3cc(Oc4c(F)cc5[nH]ccc5c4Cl)ccc3F)[nH]2)c1] | 107 |
| 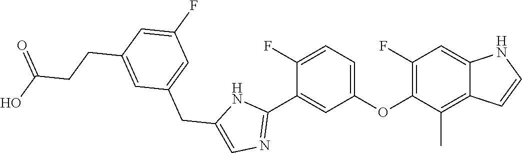 [SMILES: Cc1c(Oc2ccc(F)c(-c3ncc(Cc4cc(F)cc(CCC(=O)O)c4)[nH]3)c2)c(F)cc2[nH]ccc12] | 108 |
| 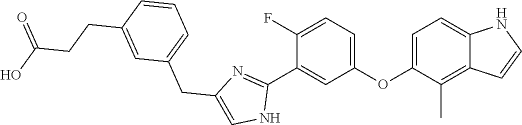 [SMILES: Cc1c(Oc2ccc(F)c(-c3nc(Cc4cccc(CCC(=O)O)c4)c[nH]3)c2)ccc2[nH]ccc12] | 109 |
| 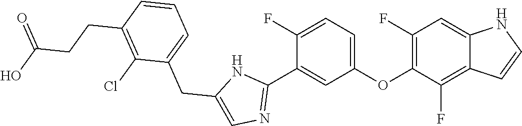 [SMILES: O=C(O)CCc1cccc(Cc2cnc(-c3cc(Oc4c(F)cc5[nH]ccc5c4F)ccc3F)[nH]2)c1Cl] | 110 |
| 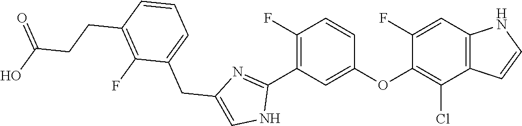 [SMILES: O=C(O)CCc1cccc(Cc2c[nH]c(-c3cc(Oc4c(F)cc5[nH]ccc5c4Cl)ccc3F)n2)c1F] | 111 |
| 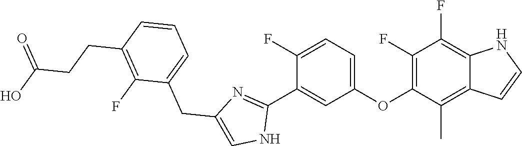 [SMILES: Cc1c(Oc2ccc(F)c(-c3nc(Cc4cccc(CCC(=O)O)c4F)c[nH]3)c2)c(F)c(F)c2[nH]ccc12] | 112 |
| 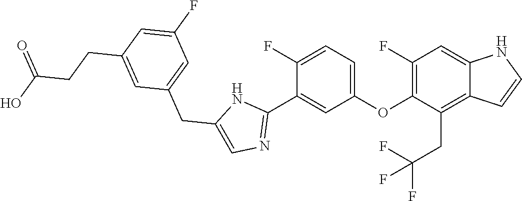 [SMILES: O=C(O)CCc1cc(F)cc(Cc2cnc(-c3cc(Oc4c(F)cc5[nH]ccc5c4CC(F)(F)F)ccc3F)[nH]2)c1] | 113 |
| 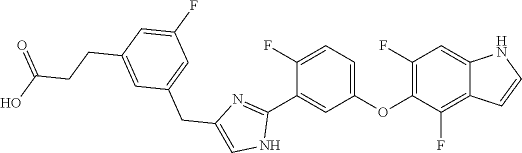 [SMILES: O=C(O)CCc1cc(F)cc(Cc2c[nH]c(-c3cc(Oc4c(F)cc5[nH]ccc5c4F)ccc3F)n2)c1] | 114 |
| 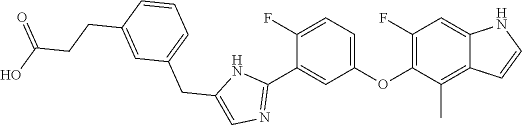 [SMILES: Cc1c(Oc2ccc(F)c(-c3ncc(Cc4cccc(CCC(=O)O)c4)[nH]3)c2)c(F)cc2[nH]ccc12] | 115 |
| 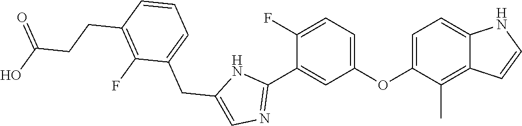 [SMILES: Cc1c(Oc2ccc(F)c(-c3ncc(Cc4cccc(CCC(=O)O)c4F)[nH]3)c2)ccc2[nH]ccc12] | 116 |
| 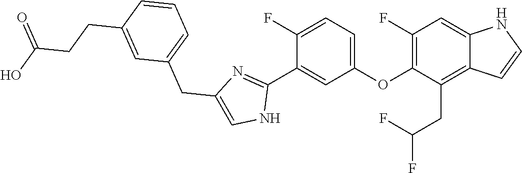 [SMILES: O=C(O)CCc1cccc(Cc2c[nH]c(-c3cc(Oc4c(F)cc5[nH]ccc5c4CC(F)F)ccc3F)n2)c1] | 117 |
| 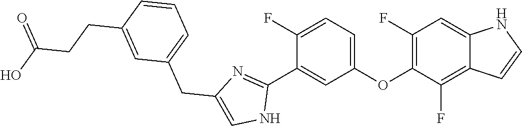 [SMILES: O=C(O)CCc1cccc(Cc2c[nH]c(-c3cc(Oc4c(F)cc5[nH]ccc5c4F)ccc3F)n2)c1] | 118 |
| 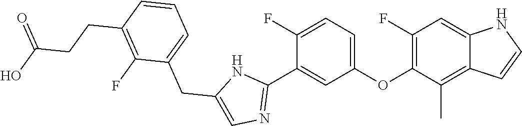 [SMILES: Cc1c(Oc2ccc(F)c(-c3ncc(Cc4cccc(CCC(=O)O)c4F)[nH]3)c2)c(F)cc2[nH]ccc12] | 119 |
| 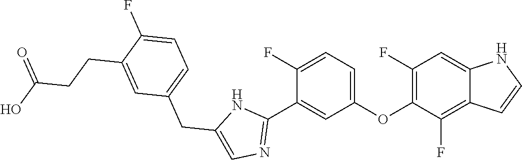 [SMILES: O=C(O)CCc1cc(Cc2cnc(-c3cc(Oc4c(F)cc5[nH]ccc5c4F)ccc3F)[nH]2)ccc1F] | 120 |
| 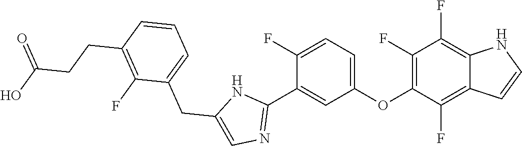 [SMILES: O=C(O)CCc1cccc(Cc2cnc(-c3cc(Oc4c(F)c(F)c5[nH]ccc5c4F)ccc3F)[nH]2)c1F] | 121 |
| 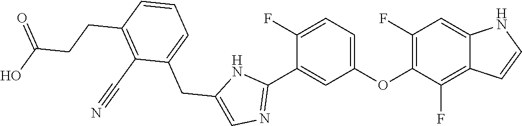 [SMILES: N#Cc1c(CCC(=O)O)cccc1Cc1cnc(-c2cc(Oc3c(F)cc4[nH]ccc4c3F)ccc2F)[nH]1] | 122 |
| 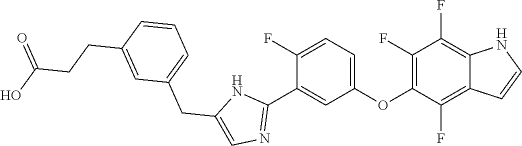 [SMILES: O=C(O)CCc1cccc(Cc2cnc(-c3cc(Oc4c(F)c(F)c5[nH]ccc5c4F)ccc3F)[nH]2)c1] | 123 |
| 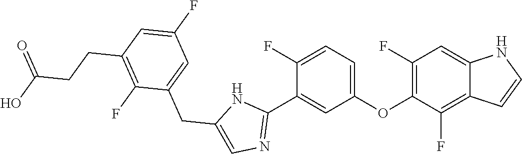 [SMILES: O=C(O)CCc1cc(F)cc(Cc2cnc(-c3cc(Oc4c(F)cc5[nH]ccc5c4F)ccc3F)[nH]2)c1F] | 124 |
| 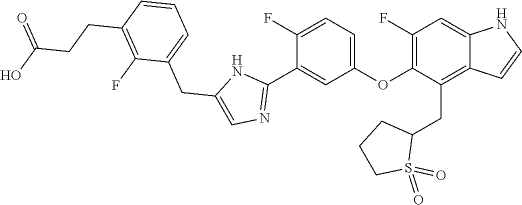 [SMILES: O=C(O)CCc1cccc(Cc2cnc(-c3cc(Oc4c(F)cc5[nH]ccc5c4CC4CCCS4(=O)=O)ccc3F)[nH]2)c1F] | 125 |
| 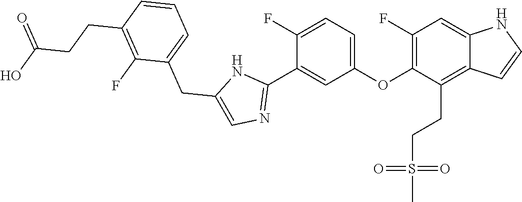 [SMILES: CS(=O)(=O)CCc1c(Oc2ccc(F)c(-c3ncc(Cc4cccc(CCC(=O)O)c4F)[nH]3)c2)c(F)cc2[nH]ccc12] | 126 |
| 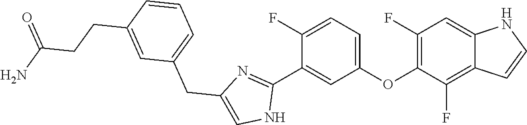 [SMILES: NC(=O)CCc1cccc(Cc2c[nH]c(-c3cc(Oc4c(F)cc5[nH]ccc5c4F)ccc3F)n2)c1] | 127 |
| 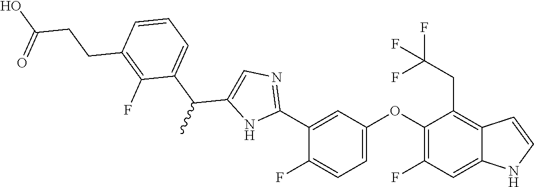 [SMILES: CC(c1cnc(-c2cc(Oc3c(F)cc4[nH]ccc4c3CC(F)(F)F)ccc2F)[nH]1)c1cccc(CCC(=O)O)c1F] | 128A |
| 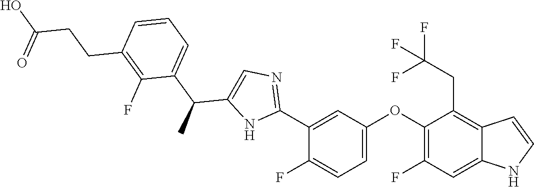 [SMILES: C[C@H](c1cnc(-c2cc(Oc3c(F)cc4[nH]ccc4c3CC(F)(F)F)ccc2F)[nH]1)c1cccc(CCC(=O)O)c1F] | 128B |
| 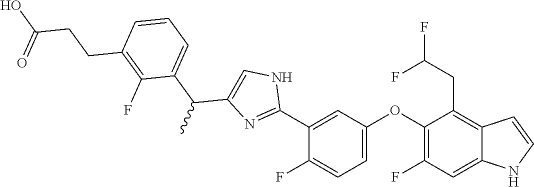 [SMILES: CC(c1c[nH]c(-c2cc(Oc3c(F)cc4[nH]ccc4c3CC(F)F)ccc2F)n1)c1cccc(CCC(=O)O)c1F] | 129A |
| 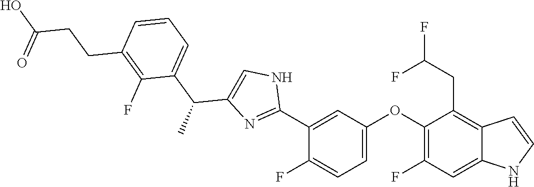 [SMILES: C[C@@H](c1c[nH]c(-c2cc(Oc3c(F)cc4[nH]ccc4c3CC(F)F)ccc2F)n1)c1cccc(CCC(=O)O)c1F] | 129B |
| 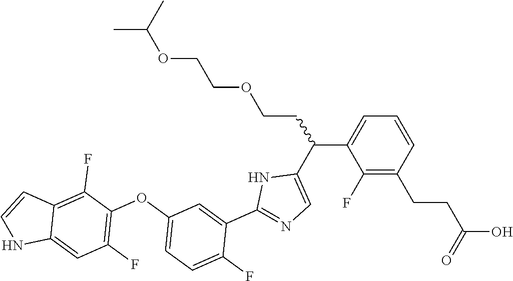 [SMILES: CC(C)OCCOCCC(c1cnc(-c2cc(Oc3c(F)cc4[nH]ccc4c3F)ccc2F)[nH]1)c1cccc(CCC(=O)O)c1F] | 130A |
| 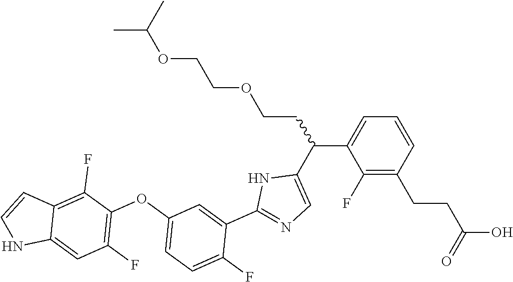 [SMILES: CC(C)OCCOCCC(c1cnc(-c2cc(Oc3c(F)cc4[nH]ccc4c3F)ccc2F)[nH]1)c1cccc(CCC(=O)O)c1F] | 130B |
| 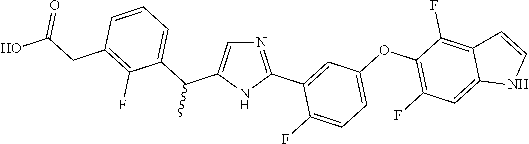 [SMILES: CC(c1cnc(-c2cc(Oc3c(F)cc4[nH]ccc4c3F)ccc2F)[nH]1)c1cccc(CC(=O)O)c1F] | 131A |
| 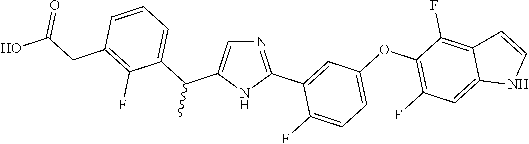 [SMILES: CC(c1cnc(-c2cc(Oc3c(F)cc4[nH]ccc4c3F)ccc2F)[nH]1)c1cccc(CC(=O)O)c1F] | 131B |
| 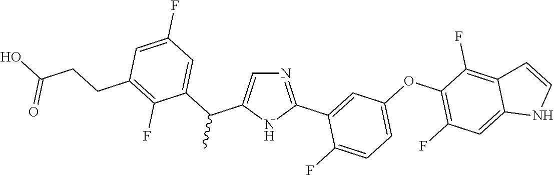 [SMILES: CC(c1cnc(-c2cc(Oc3c(F)cc4[nH]ccc4c3F)ccc2F)[nH]1)c1cc(F)cc(CCC(=O)O)c1F] | 132A |
| 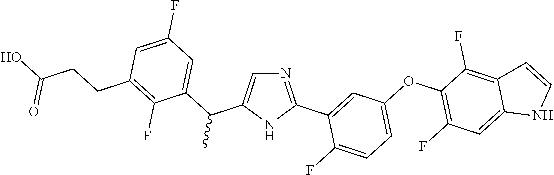 [SMILES: CC(c1cnc(-c2cc(Oc3c(F)cc4[nH]ccc4c3F)ccc2F)[nH]1)c1cc(F)cc(CCC(=O)O)c1F] | 132B |
| 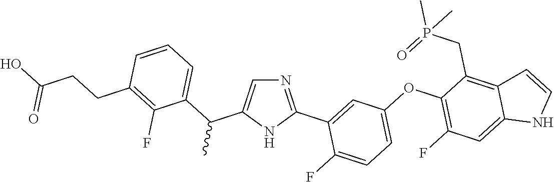 [SMILES: CC(c1cnc(-c2cc(Oc3c(F)cc4[nH]ccc4c3CP(C)(C)=O)ccc2F)[nH]1)c1cccc(CCC(=O)O)c1F] | 133A |
| 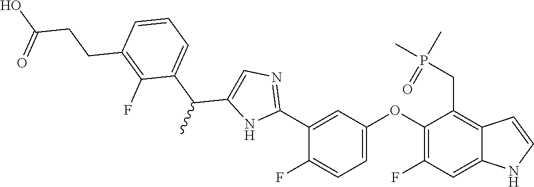 [SMILES: CC(c1cnc(-c2cc(Oc3c(F)cc4[nH]ccc4c3CP(C)(C)=O)ccc2F)[nH]1)c1cccc(CCC(=O)O)c1F] | 133B |
| 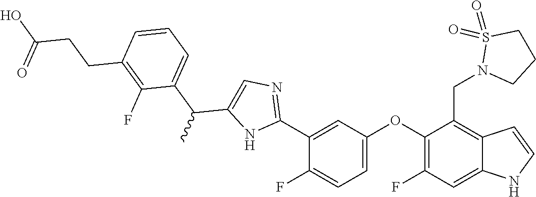 [SMILES: CC(c1cnc(-c2cc(Oc3c(F)cc4[nH]ccc4c3CN3CCCS3(=O)=O)ccc2F)[nH]1)c1cccc(CCC(=O)O)c1F] | 134A |
| 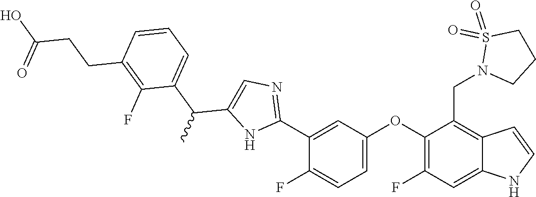 [SMILES: CC(c1cnc(-c2cc(Oc3c(F)cc4[nH]ccc4c3CN3CCCS3(=O)=O)ccc2F)[nH]1)c1cccc(CCC(=O)O)c1F] | 134B |
| 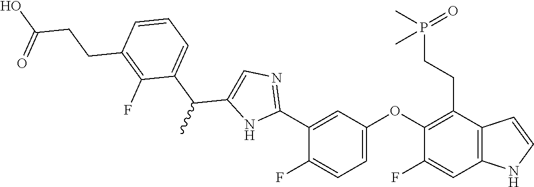 [SMILES: CC(c1cnc(-c2cc(Oc3c(F)cc4[nH]ccc4c3CCP(C)(C)=O)ccc2F)[nH]1)c1cccc(CCC(=O)O)c1F] | 135A |
| 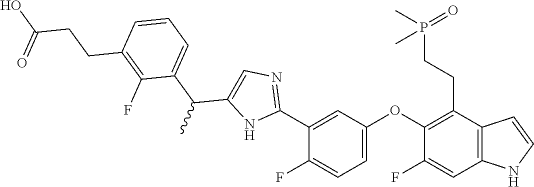 [SMILES: CC(c1cnc(-c2cc(Oc3c(F)cc4[nH]ccc4c3CCP(C)(C)=O)ccc2F)[nH]1)c1cccc(CCC(=O)O)c1F] | 135B |
| 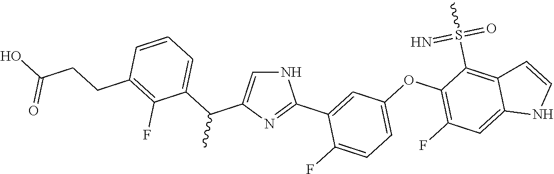 | 136A |
| 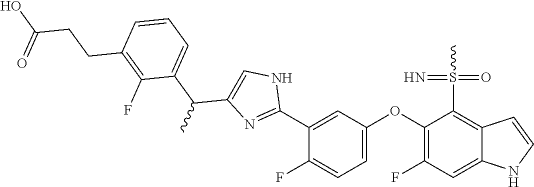 | 136B |
| 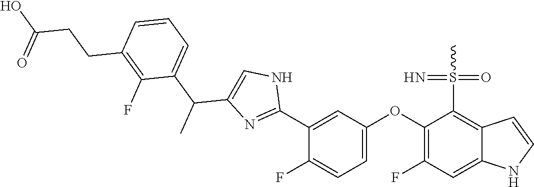 [SMILES: CC(c1c[nH]c(-c2cc(Oc3c(F)cc4[nH]ccc4c3S(C)(=N)=O)ccc2F)n1)c1cccc(CCC(=O)O)c1F] | 136C |
| 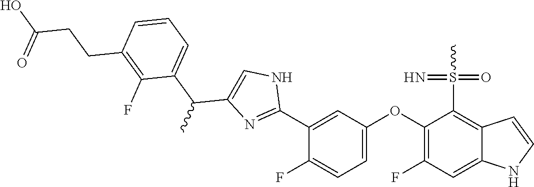 | 136D |
| 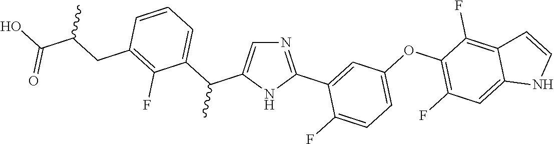 | 137A |
| 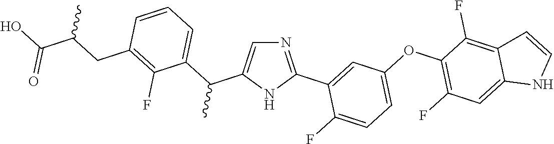 | 137B |
| 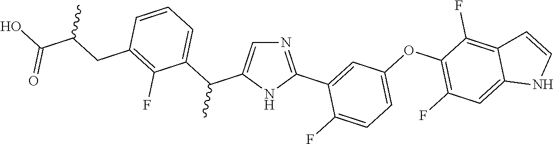 | 137C |
| 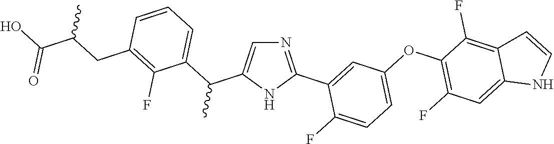 | 137D |
| 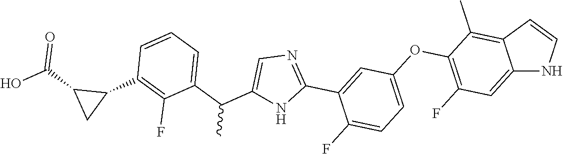 [SMILES: Cc1c(Oc2ccc(F)c(-c3ncc(C(C)c4cccc([C@@H]5C[C@@H]5C(=O)O)c4F)[nH]3)c2)c(F)cc2[nH]ccc12] | 138A |
| 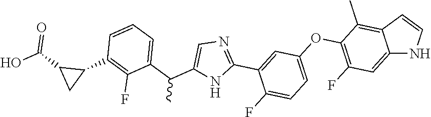 | 138B |
| 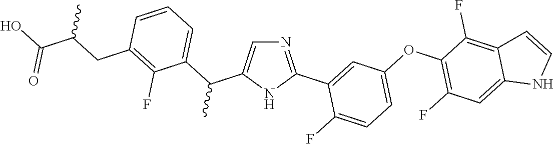 | 138C |
| 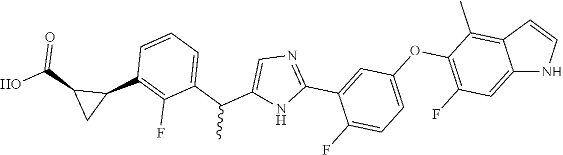 [SMILES: Cc1c(Oc2ccc(F)c(-c3ncc(C(C)c4cccc([C@H]5C[C@H]5C(=O)O)c4F)[nH]3)c2)c(F)cc2[nH]ccc12] | 138D |
| 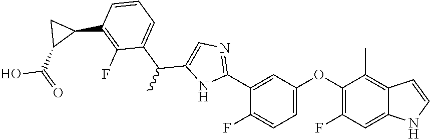 [SMILES: Cc1c(Oc2ccc(F)c(-c3ncc(C(C)c4cccc([C@@H]5C[C@H]5C(=O)O)c4F)[nH]3)c2)c(F)cc2[nH]ccc12] | 139E |
| 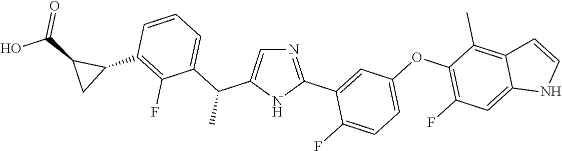 [SMILES: Cc1c(Oc2ccc(F)c(-c3ncc([C@H](C)c4cccc([C@@H]5C[C@H]5C(=O)O)c4F)[nH]3)c2)c(F)cc2[nH]ccc12] | 139F |
| 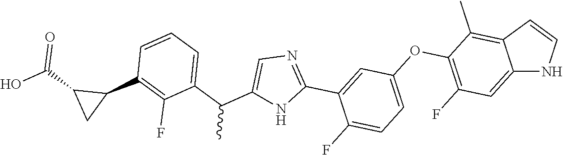 [SMILES: Cc1c(Oc2ccc(F)c(-c3ncc(C(C)c4cccc([C@H]5C[C@@H]5C(=O)O)c4F)[nH]3)c2)c(F)cc2[nH]ccc12] | 139G |
| 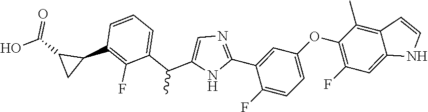 | 139H |
| 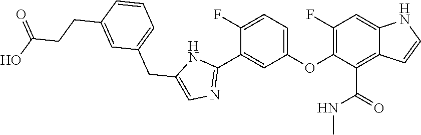 [SMILES: CNC(=O)c1c(Oc2ccc(F)c(-c3ncc(Cc4cccc(CCC(=O)O)c4)[nH]3)c2)c(F)cc2[nH]ccc12] | 140 |
| 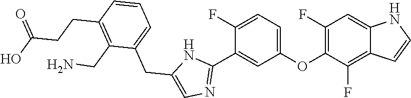 [SMILES: NCc1c(CCC(=O)O)cccc1Cc1cnc(-c2cc(Oc3c(F)cc4[nH]ccc4c3F)ccc2F)[nH]1] | 141 |
| 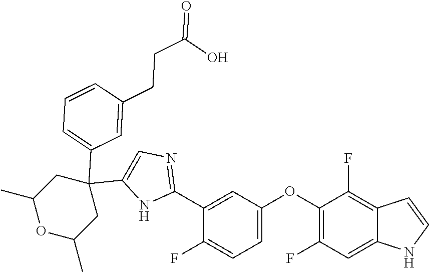 [SMILES: CC1CC(c2cccc(CCC(=O)O)c2)(c2cnc(-c3cc(Oc4c(F)cc5[nH]ccc5c4F)ccc3F)[nH]2)CC(C)O1] | 142 |
| 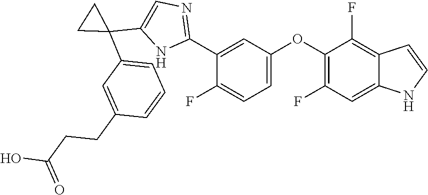 [SMILES: O=C(O)CCc1cccc(C2(c3cnc(-c4cc(Oc5c(F)cc6[nH]ccc6c5F)ccc4F)[nH]3)CC2)c1] | 143 |
| 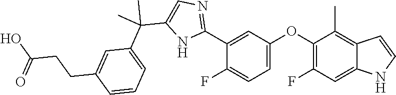 [SMILES: Cc1c(Oc2ccc(F)c(-c3ncc(C(C)(C)c4cccc(CCC(=O)O)c4)[nH]3)c2)c(F)cc2[nH]ccc12] | 144 |
| 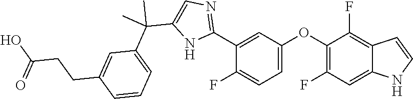 [SMILES: CC(C)(c1cccc(CCC(=O)O)c1)c1cnc(-c2cc(Oc3c(F)cc4[nH]ccc4c3F)ccc2F)[nH]1] | 145 |
| 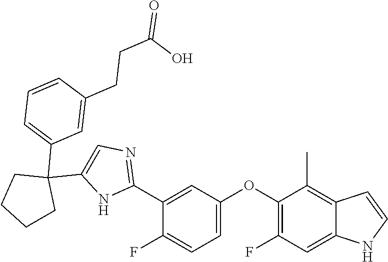 [SMILES: Cc1c(Oc2ccc(F)c(-c3ncc(C4(c5cccc(CCC(=O)O)c5)CCCC4)[nH]3)c2)c(F)cc2[nH]ccc12] | 146 |
| 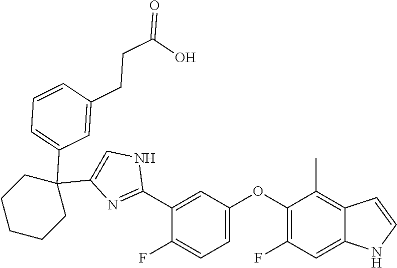 [SMILES: Cc1c(Oc2ccc(F)c(-c3nc(C4(c5cccc(CCC(=O)O)c5)CCCCC4)c[nH]3)c2)c(F)cc2[nH]ccc12] | 147 |
| 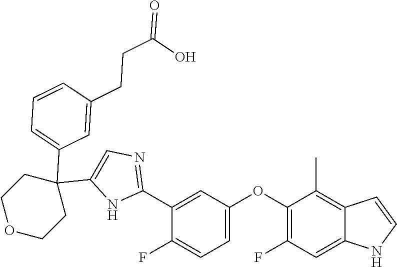 [SMILES: Cc1c(Oc2ccc(F)c(-c3ncc(C4(c5cccc(CCC(=O)O)c5)CCOCC4)[nH]3)c2)c(F)cc2[nH]ccc12] | 148 |
| 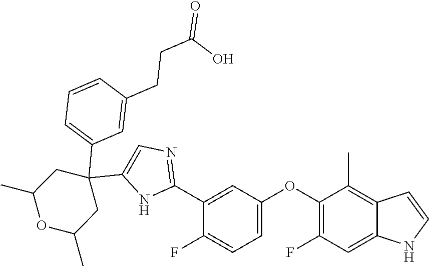 [SMILES: Cc1c(Oc2ccc(F)c(-c3ncc(C4(c5cccc(CCC(=O)O)c5)CC(C)OC(C)C4)[nH]3)c2)c(F)cc2[nH]ccc12] | 149 |
| 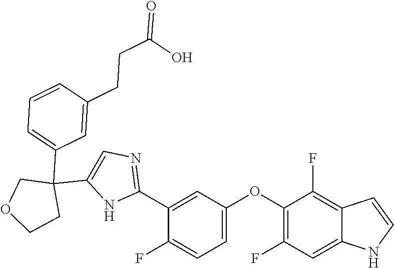 [SMILES: O=C(O)CCc1cccc(C2(c3cnc(-c4cc(Oc5c(F)cc6[nH]ccc6c5F)ccc4F)[nH]3)CCOC2)c1] | 150 |
| 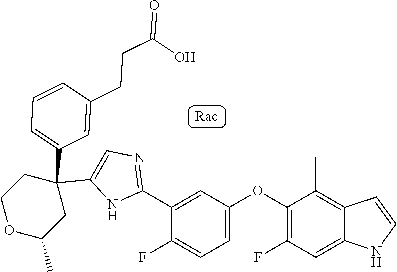 [SMILES: Cc1c(Oc2ccc(F)c(-c3ncc([C@@]4(c5cccc(CCC(=O)O)c5)CCO[C@@H](C)C4)[nH]3)c2)c(F)cc2[nH]ccc12] | 151 |
| 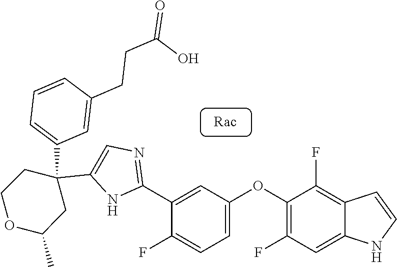 [SMILES: C[C@H]1C[C@](c2cccc(CCC(=O)O)c2)(c2cnc(-c3cc(Oc4c(F)cc5[nH]ccc5c4F)ccc3F)[nH]2)CCO1] | 152 |
| 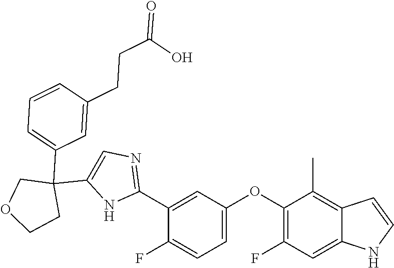 [SMILES: Cc1c(Oc2ccc(F)c(-c3ncc(C4(c5cccc(CCC(=O)O)c5)CCOC4)[nH]3)c2)c(F)cc2[nH]ccc12] | 153 |
| 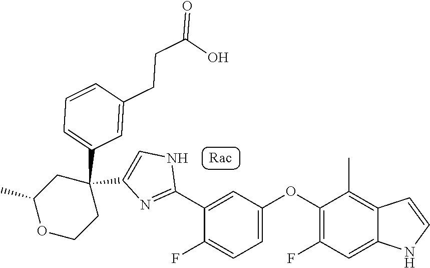 [SMILES: Cc1c(Oc2ccc(F)c(-c3nc([C@]4(c5cccc(CCC(=O)O)c5)CCO[C@H](C)C4)c[nH]3)c2)c(F)cc2[nH]ccc12] | 154 |
| 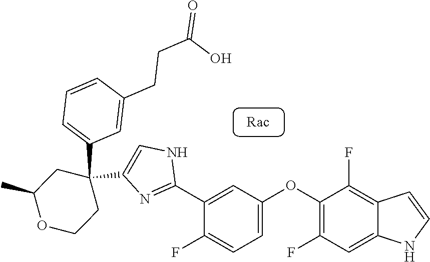 [SMILES: C[C@H]1C[C@](c2cccc(CCC(=O)O)c2)(c2c[nH]c(-c3cc(Oc4c(F)cc5[nH]ccc5c4F)ccc3F)n2)CCO1] | 155 |
| 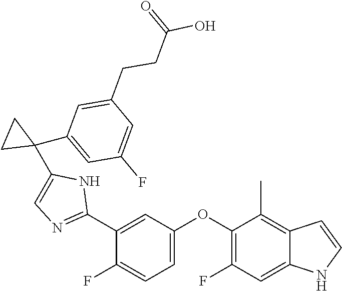 [SMILES: Cc1c(Oc2ccc(F)c(-c3ncc(C4(c5cc(F)cc(CCC(=O)O)c5)CC4)[nH]3)c2)c(F)cc2[nH]ccc12] | 156 |
| 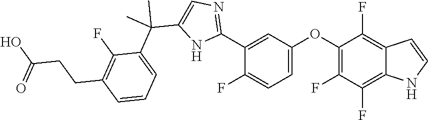 [SMILES: CC(C)(c1cnc(-c2cc(Oc3c(F)c(F)c4[nH]ccc4c3F)ccc2F)[nH]1)c1cccc(CCC(=O)O)c1F] | 157 |
| 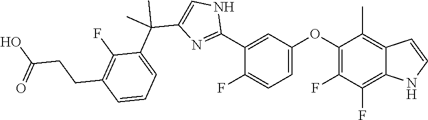 [SMILES: Cc1c(Oc2ccc(F)c(-c3nc(C(C)(C)c4cccc(CCC(=O)O)c4F)c[nH]3)c2)c(F)c(F)c2[nH]ccc12] | 158 |
| 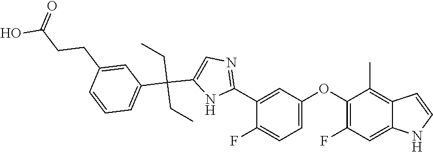 [SMILES: CCC(CC)(c1cccc(CCC(=O)O)c1)c1cnc(-c2cc(Oc3c(F)cc4[nH]ccc4c3C)ccc2F)[nH]1] | 159 |
| 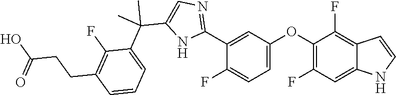 [SMILES: CC(C)(c1cnc(-c2cc(Oc3c(F)cc4[nH]ccc4c3F)ccc2F)[nH]1)c1cccc(CCC(=O)O)c1F] | 160 |
| 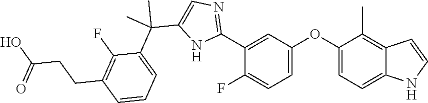 [SMILES: Cc1c(Oc2ccc(F)c(-c3ncc(C(C)(C)c4cccc(CCC(=O)O)c4F)[nH]3)c2)ccc2[nH]ccc12] | 161 |
| 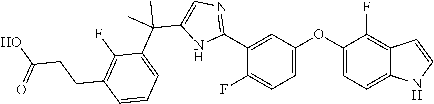 [SMILES: CC(C)(c1cnc(-c2cc(Oc3ccc4[nH]ccc4c3F)ccc2F)[nH]1)c1cccc(CCC(=O)O)c1F] | 162 |
| 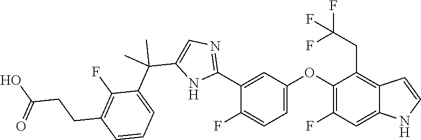 [SMILES: CC(C)(c1cnc(-c2cc(Oc3c(F)cc4[nH]ccc4c3CC(F)(F)F)ccc2F)[nH]1)c1cccc(CCC(=O)O)c1F] | 163 |
| 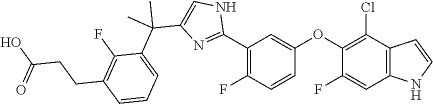 [SMILES: CC(C)(c1c[nH]c(-c2cc(Oc3c(F)cc4[nH]ccc4c3Cl)ccc2F)n1)c1cccc(CCC(=O)O)c1F] | 164 |
| 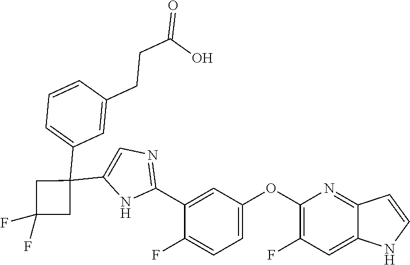 [SMILES: O=C(O)CCc1cccc(C2(c3cnc(-c4cc(Oc5nc6cc[nH]c6cc5F)ccc4F)[nH]3)CC(F)(F)C2)c1] | 165 |
| 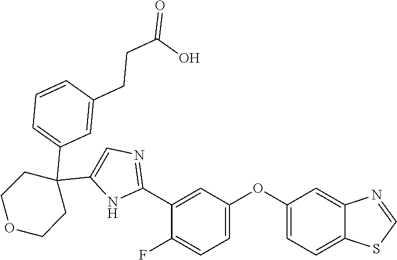 [SMILES: O=C(O)CCc1cccc(C2(c3cnc(-c4cc(Oc5ccc6scnc6c5)ccc4F)[nH]3)CCOCC2)c1] | 166 |
| 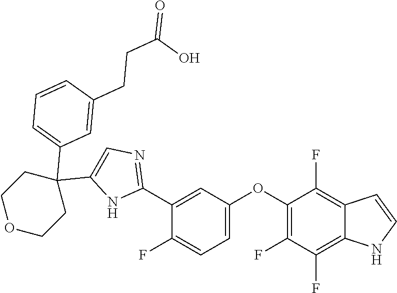 [SMILES: O=C(O)CCc1cccc(C2(c3cnc(-c4cc(Oc5c(F)c(F)c6[nH]ccc6c5F)ccc4F)[nH]3)CCOCC2)c1] | 167 |
| 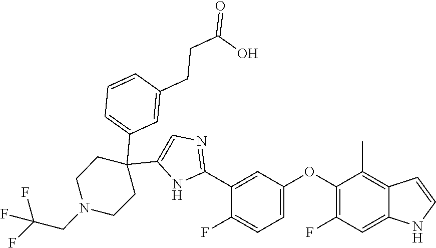 [SMILES: Cc1c(Oc2ccc(F)c(-c3ncc(C4(c5cccc(CCC(=O)O)c5)CCN(CC(F)(F)F)CC4)[nH]3)c2)c(F)cc2[nH]ccc12] | 168 |
| 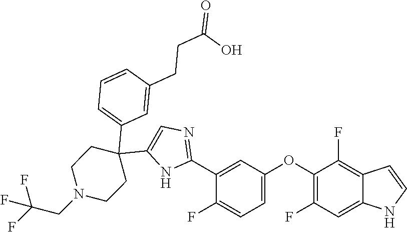 [SMILES: O=C(O)CCc1cccc(C2(c3cnc(-c4cc(Oc5c(F)cc6[nH]ccc6c5F)ccc4F)[nH]3)CCN(CC(F)(F)F)CC2)c1] | 169 |
| 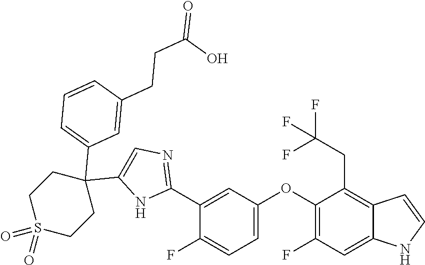 [SMILES: O=C(O)CCc1cccc(C2(c3cnc(-c4cc(Oc5c(F)cc6[nH]ccc6c5CC(F)(F)F)ccc4F)[nH]3)CCS(=O)(=O)CC2)c1] | 170 |
| 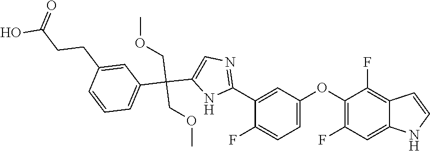 [SMILES: COCC(COC)(c1cccc(CCC(=O)O)c1)c1cnc(-c2cc(Oc3c(F)cc4[nH]ccc4c3F)ccc2F)[nH]1] | 171 |
| 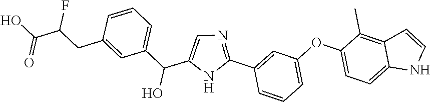 [SMILES: Cc1c(Oc2cccc(-c3ncc(C(O)c4cccc(CC(F)C(=O)O)c4)[nH]3)c2)ccc2[nH]ccc12] | 172 |
| 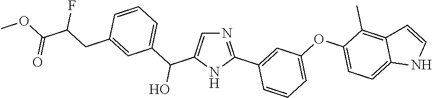 [SMILES: COC(=O)C(F)Cc1cccc(C(O)c2cnc(-c3cccc(Oc4ccc5[nH]ccc5c4C)c3)[nH]2)c1] | 173 |
| 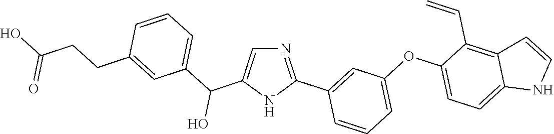 [SMILES: C=Cc1c(Oc2cccc(-c3ncc(C(O)c4cccc(CCC(=O)O)c4)[nH]3)c2)ccc2[nH]ccc12] | 174 |
| 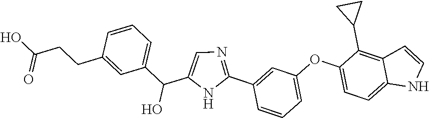 [SMILES: O=C(O)CCc1cccc(C(O)c2cnc(-c3cccc(Oc4ccc5[nH]ccc5c4C4CC4)c3)[nH]2)c1] | 175 |
| 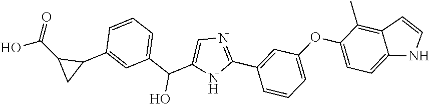 [SMILES: Cc1c(Oc2cccc(-c3ncc(C(O)c4cccc(C5CC5C(=O)O)c4)[nH]3)c2)ccc2[nH]ccc12] | 176 |
| 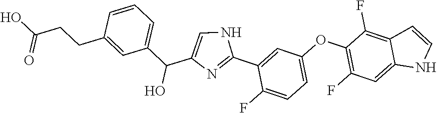 [SMILES: O=C(O)CCc1cccc(C(O)c2c[nH]c(-c3cc(Oc4c(F)cc5[nH]ccc5c4F)ccc3F)n2)c1] | 177 |
| 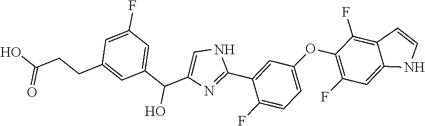 [SMILES: O=C(O)CCc1cc(F)cc(C(O)c2c[nH]c(-c3cc(Oc4c(F)cc5[nH]ccc5c4F)ccc3F)n2)c1] | 178 |
| 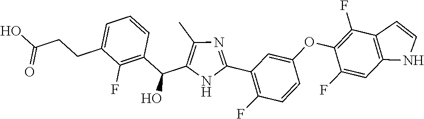 [SMILES: Cc1nc(-c2cc(Oc3c(F)cc4[nH]ccc4c3F)ccc2F)[nH]c1[C@@H](O)c1cccc(CCC(=O)O)c1F] | 179 |
| 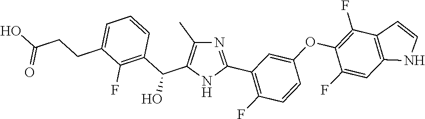 [SMILES: Cc1nc(-c2cc(Oc3c(F)cc4[nH]ccc4c3F)ccc2F)[nH]c1[C@H](O)c1cccc(CCC(=O)O)c1F] | 180 |
| 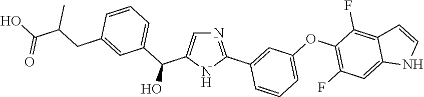 [SMILES: CC(Cc1cccc([C@H](O)c2cnc(-c3cccc(Oc4c(F)cc5[nH]ccc5c4F)c3)[nH]2)c1)C(=O)O] | 181 |
| 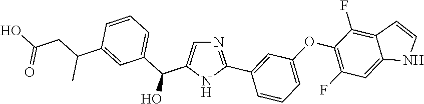 [SMILES: CC(CC(=O)O)c1cccc([C@H](O)c2cnc(-c3cccc(Oc4c(F)cc5[nH]ccc5c4F)c3)[nH]2)c1] | 182 |
| 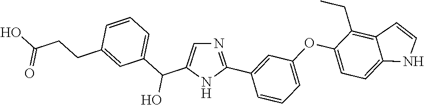 [SMILES: CCc1c(Oc2cccc(-c3ncc(C(O)c4cccc(CCC(=O)O)c4)[nH]3)c2)ccc2[nH]ccc12] | 183 |
| 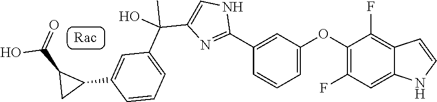 [SMILES: CC(O)(c1cccc([C@@H]2C[C@H]2C(=O)O)c1)c1c[nH]c(-c2cccc(Oc3c(F)cc4[nH]ccc4c3F)c2)n1] | 184 |
| 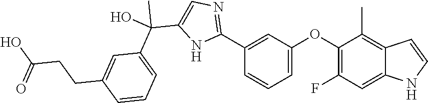 [SMILES: Cc1c(Oc2cccc(-c3ncc(C(C)(O)c4cccc(CCC(=O)O)c4)[nH]3)c2)c(F)cc2[nH]ccc12] | 185 |
| 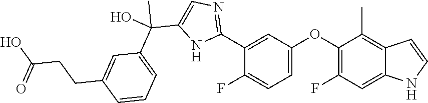 [SMILES: Cc1c(Oc2ccc(F)c(-c3ncc(C(C)(O)c4cccc(CCC(=O)O)c4)[nH]3)c2)c(F)cc2[nH]ccc12] | 186 |
| 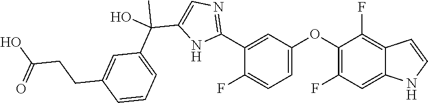 [SMILES: CC(O)(c1cccc(CCC(=O)O)c1)c1cnc(-c2cc(Oc3c(F)cc4[nH]ccc4c3F)ccc2F)[nH]1] | 187 |
| 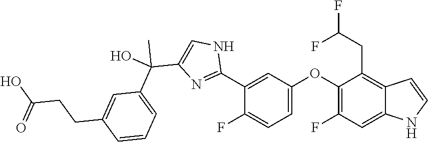 [SMILES: CC(O)(c1cccc(CCC(=O)O)c1)c1c[nH]c(-c2cc(Oc3c(F)cc4[nH]ccc4c3CC(F)F)ccc2F)n1] | 188 |
| 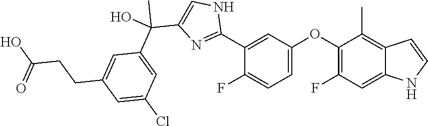 [SMILES: Cc1c(Oc2ccc(F)c(-c3nc(C(C)(O)c4cc(Cl)cc(CCC(=O)O)c4)c[nH]3)c2)c(F)cc2[nH]ccc12] | 189 |
| 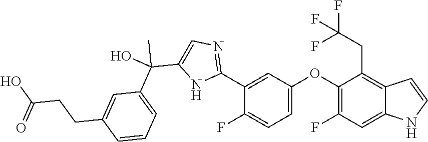 [SMILES: CC(O)(c1cccc(CCC(=O)O)c1)c1cnc(-c2cc(Oc3c(F)cc4[nH]ccc4c3CC(F)(F)F)ccc2F)[nH]1] | 190 |
| 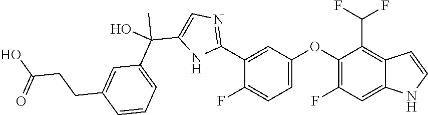 [SMILES: CC(O)(c1cccc(CCC(=O)O)c1)c1cnc(-c2cc(Oc3c(F)cc4[nH]ccc4c3C(F)F)ccc2F)[nH]1] | 191 |
| 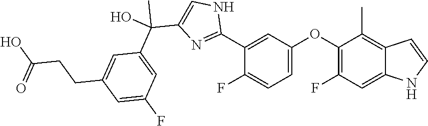 [SMILES: Cc1c(Oc2ccc(F)c(-c3nc(C(C)(O)c4cc(F)cc(CCC(=O)O)c4)c[nH]3)c2)c(F)cc2[nH]ccc12] | 192 |
| 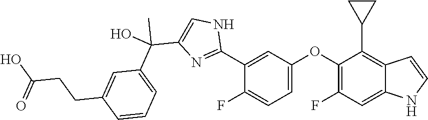 [SMILES: CC(O)(c1cccc(CCC(=O)O)c1)c1c[nH]c(-c2cc(Oc3c(F)cc4[nH]ccc4c3C3CC3)ccc2F)n1] | 193 |
| 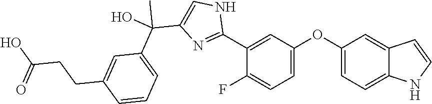 [SMILES: CC(O)(c1cccc(CCC(=O)O)c1)c1c[nH]c(-c2cc(Oc3ccc4[nH]ccc4c3)ccc2F)n1] | 194 |
| 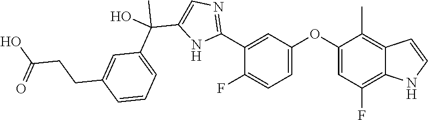 [SMILES: Cc1c(Oc2ccc(F)c(-c3ncc(C(C)(O)c4cccc(CCC(=O)O)c4)[nH]3)c2)cc(F)c2[nH]ccc12] | 195 |
| 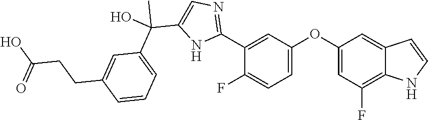 [SMILES: CC(O)(c1cccc(CCC(=O)O)c1)c1cnc(-c2cc(Oc3cc(F)c4[nH]ccc4c3)ccc2F)[nH]1] | 196 |
| 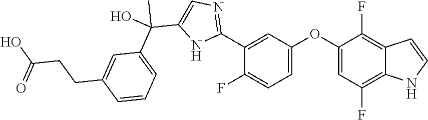 [SMILES: CC(O)(c1cccc(CCC(=O)O)c1)c1cnc(-c2cc(Oc3cc(F)c4[nH]ccc4c3F)ccc2F)[nH]1] | 197 |
| 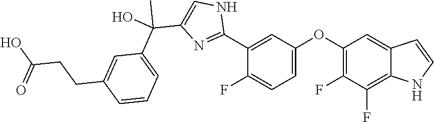 [SMILES: CC(O)(c1cccc(CCC(=O)O)c1)c1c[nH]c(-c2cc(Oc3cc4cc[nH]c4c(F)c3F)ccc2F)n1] | 198 |
| 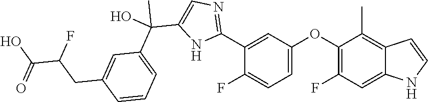 [SMILES: Cc1c(Oc2ccc(F)c(-c3ncc(C(C)(O)c4cccc(CC(F)C(=O)O)c4)[nH]3)c2)c(F)cc2[nH]ccc12] | 199 |
| 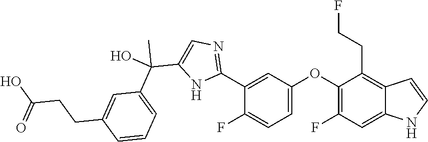 [SMILES: CC(O)(c1cccc(CCC(=O)O)c1)c1cnc(-c2cc(Oc3c(F)cc4[nH]ccc4c3CCF)ccc2F)[nH]1] | 200 |
| 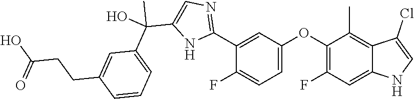 [SMILES: Cc1c(Oc2ccc(F)c(-c3ncc(C(C)(O)c4cccc(CCC(=O)O)c4)[nH]3)c2)c(F)cc2[nH]cc(Cl)c12] | 201 |
| 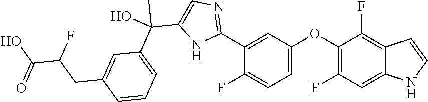 [SMILES: CC(O)(c1cccc(CC(F)C(=O)O)c1)c1cnc(-c2cc(Oc3c(F)cc4[nH]ccc4c3F)ccc2F)[nH]1] | 202 |
| 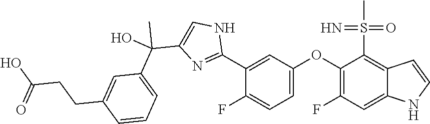 [SMILES: CC(O)(c1cccc(CCC(=O)O)c1)c1c[nH]c(-c2cc(Oc3c(F)cc4[nH]ccc4c3S(C)(=N)=O)ccc2F)n1] | 203 |
| 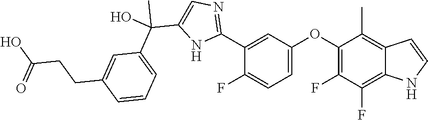 [SMILES: Cc1c(Oc2ccc(F)c(-c3ncc(C(C)(O)c4cccc(CCC(=O)O)c4)[nH]3)c2)c(F)c(F)c2[nH]ccc12] | 204 |
| 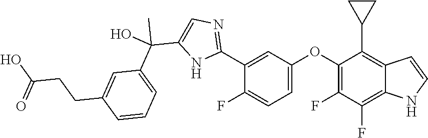 [SMILES: CC(O)(c1cccc(CCC(=O)O)c1)c1cnc(-c2cc(Oc3c(F)c(F)c4[nH]ccc4c3C3CC3)ccc2F)[nH]1] | 205 |
| 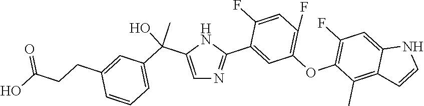 [SMILES: Cc1c(Oc2cc(-c3ncc(C(C)(O)c4cccc(CCC(=O)O)c4)[nH]3)c(F)cc2F)c(F)cc2[nH]ccc12] | 206 |
| 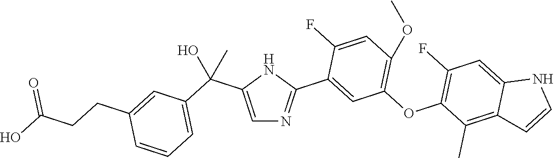 [SMILES: COc1cc(F)c(-c2ncc(C(C)(O)c3cccc(CCC(=O)O)c3)[nH]2)cc1Oc1c(F)cc2[nH]ccc2c1C] | 281 |
| 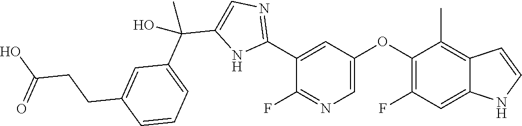 [SMILES: Cc1c(Oc2cnc(F)c(-c3ncc(C(C)(O)c4cccc(CCC(=O)O)c4)[nH]3)c2)c(F)cc2[nH]ccc12] | 207 |
| 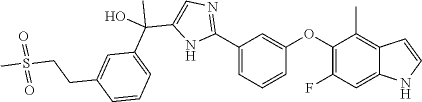 [SMILES: Cc1c(Oc2cccc(-c3ncc(C(C)(O)c4cccc(CCS(C)(=O)=O)c4)[nH]3)c2)c(F)cc2[nH]ccc12] | 208 |
| 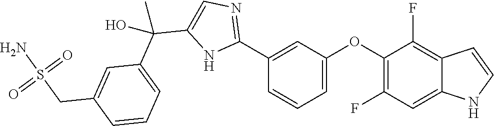 [SMILES: CC(O)(c1cccc(CS(N)(=O)=O)c1)c1cnc(-c2cccc(Oc3c(F)cc4[nH]ccc4c3F)c2)[nH]1] | 209 |
| 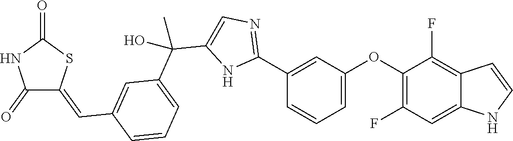 [SMILES: CC(O)(c1cccc(/C=C2\SC(=O)NC2=O)c1)c1cnc(-c2cccc(Oc3c(F)cc4[nH]ccc4c3F)c2)[nH]1] | 210 |
| 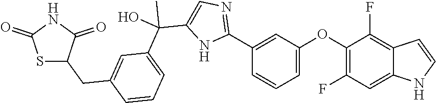 [SMILES: CC(O)(c1cccc(CC2SC(=O)NC2=O)c1)c1cnc(-c2cccc(Oc3c(F)cc4[nH]ccc4c3F)c2)[nH]1] | 211 |
| 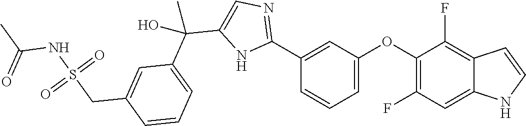 [SMILES: CC(=O)NS(=O)(=O)Cc1cccc(C(C)(O)c2cnc(-c3cccc(Oc4c(F)cc5[nH]ccc5c4F)c3)[nH]2)c1] | 212 |
| 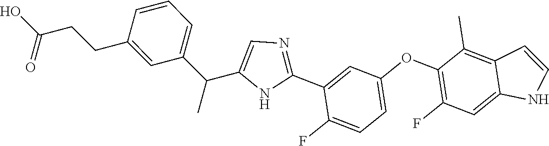 [SMILES: Cc1c(Oc2ccc(F)c(-c3ncc(C(C)c4cccc(CCC(=O)O)c4)[nH]3)c2)c(F)cc2[nH]ccc12] | 213 |
| 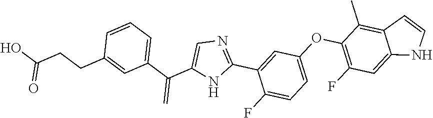 [SMILES: C=C(c1cccc(CCC(=O)O)c1)c1cnc(-c2cc(Oc3c(F)cc4[nH]ccc4c3C)ccc2F)[nH]1] | 214 |
| 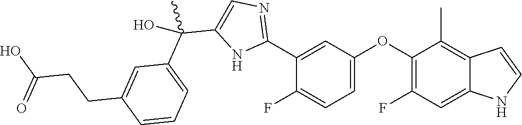 [SMILES: Cc1c(Oc2ccc(F)c(-c3ncc(C(C)(O)c4cccc(CCC(=O)O)c4)[nH]3)c2)c(F)cc2[nH]ccc12] | 215A |
| 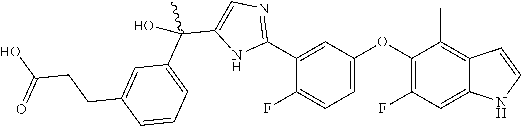 [SMILES: Cc1c(Oc2ccc(F)c(-c3ncc(C(C)(O)c4cccc(CCC(=O)O)c4)[nH]3)c2)c(F)cc2[nH]ccc12] | 215B |
| 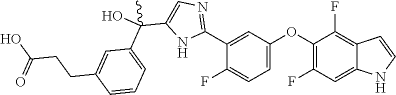 [SMILES: CC(O)(c1cccc(CCC(=O)O)c1)c1cnc(-c2cc(Oc3c(F)cc4[nH]ccc4c3F)ccc2F)[nH]1] | 216A |
| 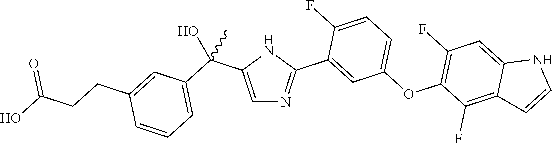 [SMILES: CC(O)(c1cccc(CCC(=O)O)c1)c1cnc(-c2cc(Oc3c(F)cc4[nH]ccc4c3F)ccc2F)[nH]1] | 216B |
| 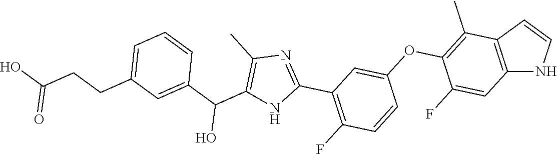 [SMILES: Cc1nc(-c2cc(Oc3c(F)cc4[nH]ccc4c3C)ccc2F)[nH]c1C(O)c1cccc(CCC(=O)O)c1] | 217 |
| 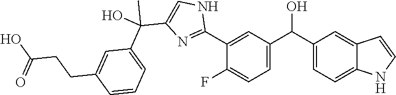 [SMILES: CC(O)(c1cccc(CCC(=O)O)c1)c1c[nH]c(-c2cc(C(O)c3ccc4[nH]ccc4c3)ccc2F)n1] | 218 |
| 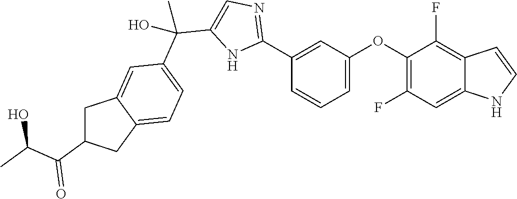 [SMILES: C[C@@H](O)C(=O)C1Cc2ccc(C(C)(O)c3cnc(-c4cccc(Oc5c(F)cc6[nH]ccc6c5F)c4)[nH]3)cc2C1] | 219 |
| 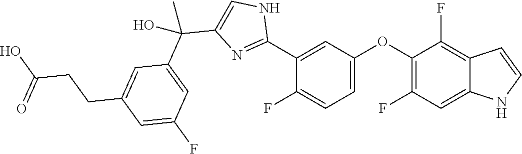 [SMILES: CC(O)(c1cc(F)cc(CCC(=O)O)c1)c1c[nH]c(-c2cc(Oc3c(F)cc4[nH]ccc4c3F)ccc2F)n1] | 220 |
| 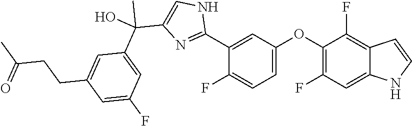 [SMILES: CC(=O)CCc1cc(F)cc(C(C)(O)c2c[nH]c(-c3cc(Oc4c(F)cc5[nH]ccc5c4F)ccc3F)n2)c1] | 221 |
| 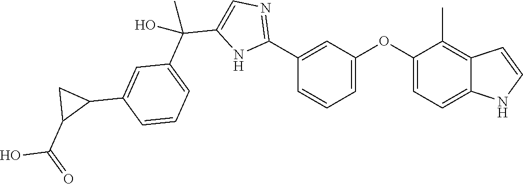 [SMILES: Cc1c(Oc2cccc(-c3ncc(C(C)(O)c4cccc(C5CC5C(=O)O)c4)[nH]3)c2)ccc2[nH]ccc12] | 222 |
| 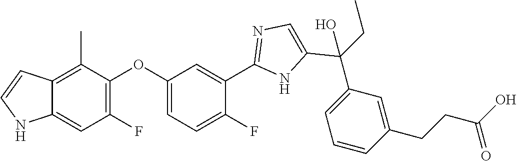 [SMILES: CCC(O)(c1cccc(CCC(=O)O)c1)c1cnc(-c2cc(Oc3c(F)cc4[nH]ccc4c3C)ccc2F)[nH]1] | 223 |
| 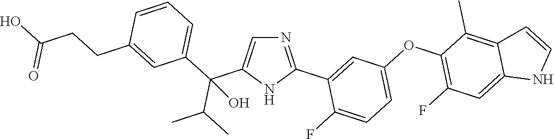 [SMILES: Cc1c(Oc2ccc(F)c(-c3ncc(C(O)(c4cccc(CCC(=O)O)c4)C(C)C)[nH]3)c2)c(F)cc2[nH]ccc12] | 224 |
| 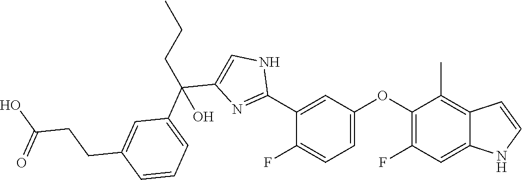 [SMILES: CCCC(O)(c1cccc(CCC(=O)O)c1)c1c[nH]c(-c2cc(Oc3c(F)cc4[nH]ccc4c3C)ccc2F)n1] | 225 |
| 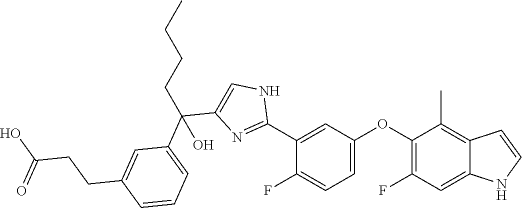 [SMILES: CCCCC(O)(c1cccc(CCC(=O)O)c1)c1c[nH]c(-c2cc(Oc3c(F)cc4[nH]ccc4c3C)ccc2F)n1] | 226 |
| 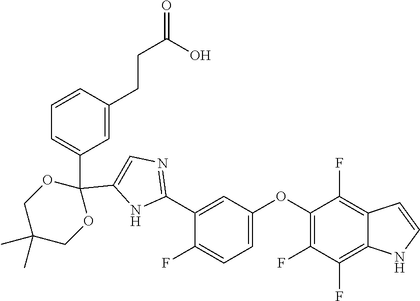 [SMILES: CC1(C)COC(c2cccc(CCC(=O)O)c2)(c2cnc(-c3cc(Oc4c(F)c(F)c5[nH]ccc5c4F)ccc3F)[nH]2)OC1] | 227 |
| 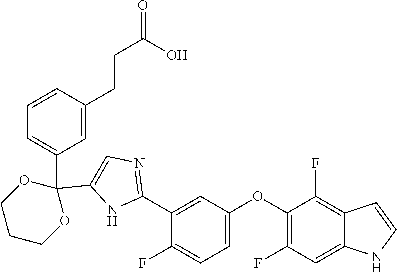 [SMILES: O=C(O)CCc1cccc(C2(c3cnc(-c4cc(Oc5c(F)cc6[nH]ccc6c5F)ccc4F)[nH]3)OCCCO2)c1] | 228 |
| 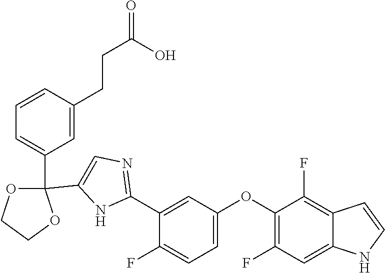 [SMILES: O=C(O)CCc1cccc(C2(c3cnc(-c4cc(Oc5c(F)cc6[nH]ccc6c5F)ccc4F)[nH]3)OCCO2)c1] | 229 |
| 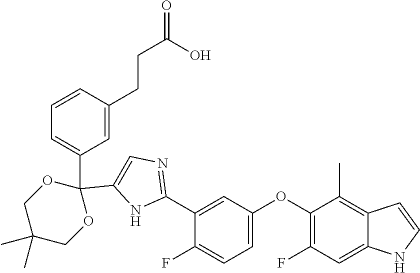 [SMILES: Cc1c(Oc2ccc(F)c(-c3ncc(C4(c5cccc(CCC(=O)O)c5)OCC(C)(C)CO4)[nH]3)c2)c(F)cc2[nH]ccc12] | 230 |
| 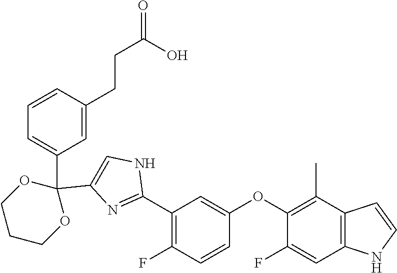 [SMILES: Cc1c(Oc2ccc(F)c(-c3nc(C4(c5cccc(CCC(=O)O)c5)OCCCO4)c[nH]3)c2)c(F)cc2[nH]ccc12] | 231 |
| 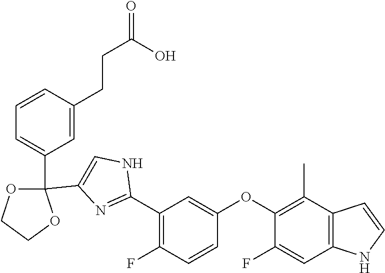 [SMILES: Cc1c(Oc2ccc(F)c(-c3nc(C4(c5cccc(CCC(=O)O)c5)OCCO4)c[nH]3)c2)c(F)cc2[nH]ccc12] | 232 |
| 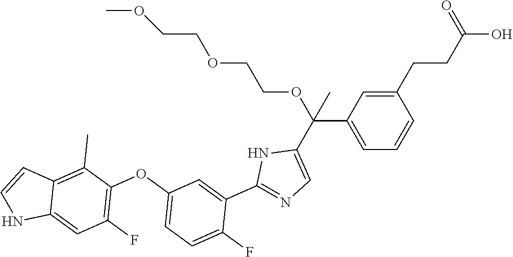 [SMILES: COCCOCCOC(C)(c1cccc(CCC(=O)O)c1)c1cnc(-c2cc(Oc3c(F)cc4[nH]ccc4c3C)ccc2F)[nH]1] | 233 |
| 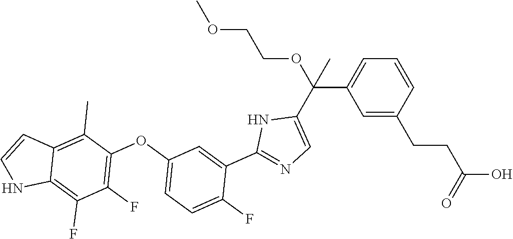 [SMILES: COCCOC(C)(c1cccc(CCC(=O)O)c1)c1cnc(-c2cc(Oc3c(F)c(F)c4[nH]ccc4c3C)ccc2F)[nH]1] | 234 |
| 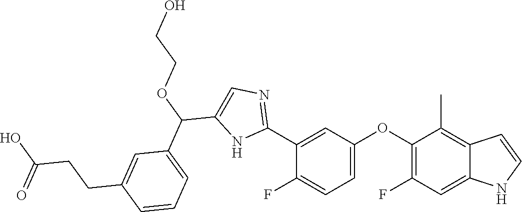 [SMILES: Cc1c(Oc2ccc(F)c(-c3ncc(C(OCCO)c4cccc(CCC(=O)O)c4)[nH]3)c2)c(F)cc2[nH]ccc12] | 235 |
| 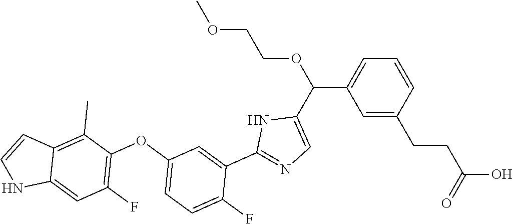 [SMILES: COCCOC(c1cccc(CCC(=O)O)c1)c1cnc(-c2cc(Oc3c(F)cc4[nH]ccc4c3C)ccc2F)[nH]1] | 236 |
| 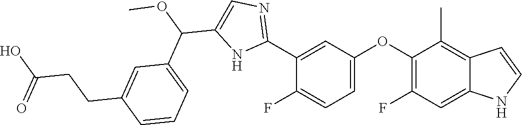 [SMILES: COC(c1cccc(CCC(=O)O)c1)c1cnc(-c2cc(Oc3c(F)cc4[nH]ccc4c3C)ccc2F)[nH]1] | 237 |
| 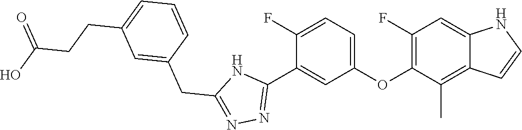 [SMILES: Cc1c(Oc2ccc(F)c(-c3nnc(Cc4cccc(CCC(=O)O)c4)[nH]3)c2)c(F)cc2[nH]ccc12] | 238 |
| 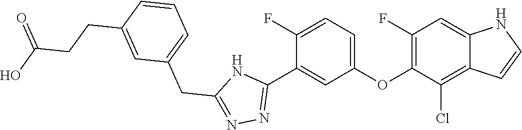 [SMILES: O=C(O)CCc1cccc(Cc2nnc(-c3cc(Oc4c(F)cc5[nH]ccc5c4Cl)ccc3F)[nH]2)c1] | 239 |
| 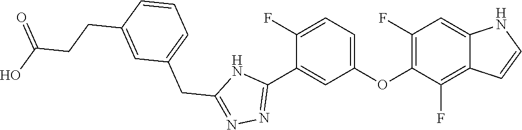 [SMILES: O=C(O)CCc1cccc(Cc2nnc(-c3cc(Oc4c(F)cc5[nH]ccc5c4F)ccc3F)[nH]2)c1] | 240 |
| 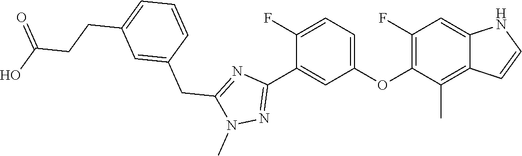 [SMILES: Cc1c(Oc2ccc(F)c(-c3nc(Cc4cccc(CCC(=O)O)c4)n(C)n3)c2)c(F)cc2[nH]ccc12] | 241 |
| 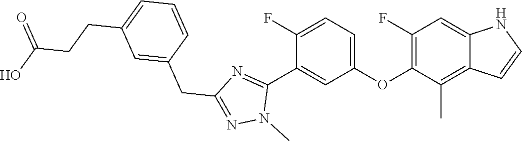 [SMILES: Cc1c(Oc2ccc(F)c(-c3nc(Cc4cccc(CCC(=O)O)c4)nn3C)c2)c(F)cc2[nH]ccc12] | 242 |
| 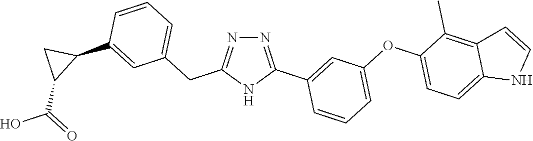 [SMILES: Cc1c(Oc2cccc(-c3nnc(Cc4cccc([C@@H]5C[C@H]5C(=O)O)c4)[nH]3)c2)ccc2[nH]ccc12] | 243 |
| 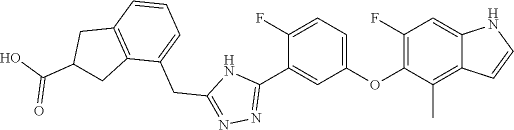 [SMILES: Cc1c(Oc2ccc(F)c(-c3nnc(Cc4cccc5c4CC(C(=O)O)C5)[nH]3)c2)c(F)cc2[nH]ccc12] | 244 |
| 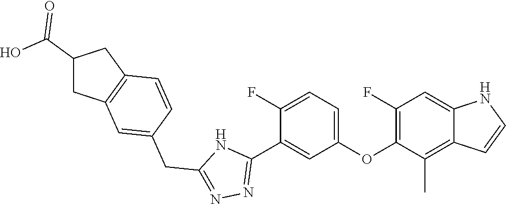 [SMILES: Cc1c(Oc2ccc(F)c(-c3nnc(Cc4ccc5c(c4)CC(C(=O)O)C5)[nH]3)c2)c(F)cc2[nH]ccc12] | 245 |
| 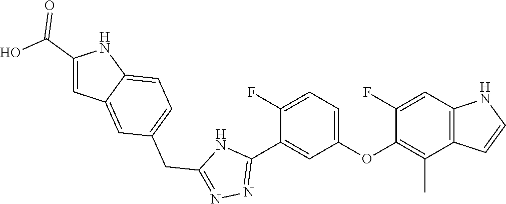 [SMILES: Cc1c(Oc2ccc(F)c(-c3nnc(Cc4ccc5[nH]c(C(=O)O)cc5c4)[nH]3)c2)c(F)cc2[nH]ccc12] | 246 |
| 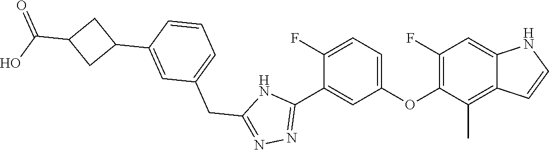 [SMILES: Cc1c(Oc2ccc(F)c(-c3nnc(Cc4cccc(C5CC(C(=O)O)C5)c4)[nH]3)c2)c(F)cc2[nH]ccc12] | 247 |
| 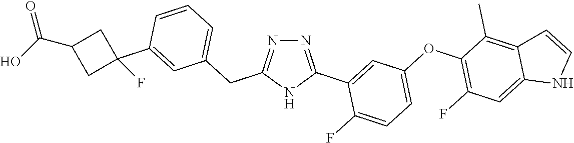 [SMILES: Cc1c(Oc2ccc(F)c(-c3nnc(Cc4cccc(C5(F)CC(C(=O)O)C5)c4)[nH]3)c2)c(F)cc2[nH]ccc12] | 248 |
| 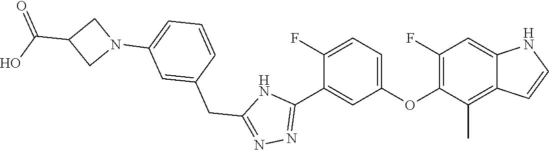 [SMILES: Cc1c(Oc2ccc(F)c(-c3nnc(Cc4cccc(N5CC(C(=O)O)C5)c4)[nH]3)c2)c(F)cc2[nH]ccc12] | 249 |
| 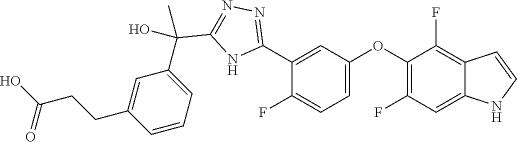 [SMILES: CC(O)(c1cccc(CCC(=O)O)c1)c1nnc(-c2cc(Oc3c(F)cc4[nH]ccc4c3F)ccc2F)[nH]1] | 250 |
| 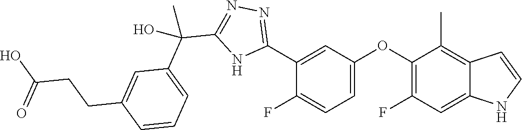 [SMILES: Cc1c(Oc2ccc(F)c(-c3nnc(C(C)(O)c4cccc(CCC(=O)O)c4)[nH]3)c2)c(F)cc2[nH]ccc12] | 251 |
| 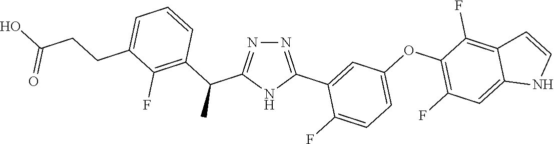 [SMILES: C[C@H](c1nnc(-c2cc(Oc3c(F)cc4[nH]ccc4c3F)ccc2F)[nH]1)c1cccc(CCC(=O)O)c1F] | 252A |
| 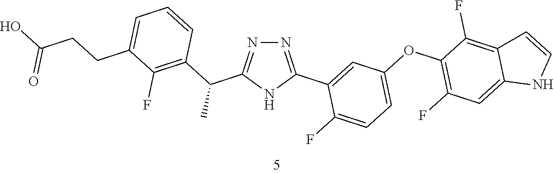 [SMILES: C[C@@H](c1nnc(-c2cc(Oc3c(F)cc4[nH]ccc4c3F)ccc2F)[nH]1)c1cccc(CCC(=O)O)c1F] | 252B |
| 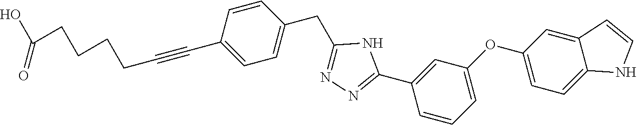 [SMILES: O=C(O)CCCCC#Cc1ccc(Cc2nnc(-c3cccc(Oc4ccc5[nH]ccc5c4)c3)[nH]2)cc1] | 253 |
| 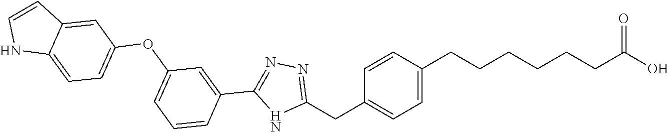 [SMILES: O=C(O)CCCCCCc1ccc(Cc2nnc(-c3cccc(Oc4ccc5[nH]ccc5c4)c3)[nH]2)cc1] | 254 |
| 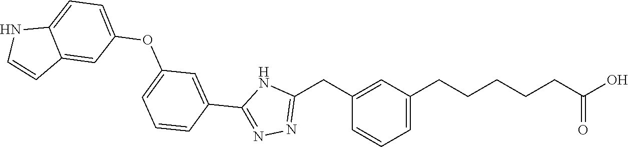 [SMILES: O=C(O)CCCCCc1cccc(Cc2nnc(-c3cccc(Oc4ccc5[nH]ccc5c4)c3)[nH]2)c1] | 255 |
| 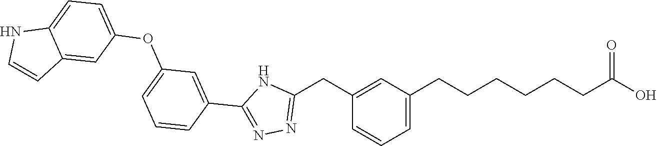 [SMILES: O=C(O)CCCCCCc1cccc(Cc2nnc(-c3cccc(Oc4ccc5[nH]ccc5c4)c3)[nH]2)c1] | 256 |
| 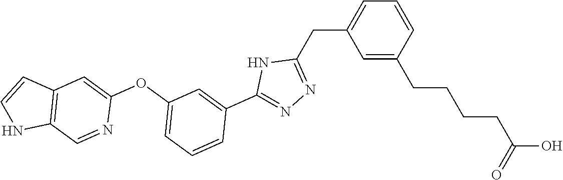 [SMILES: O=C(O)CCCCc1cccc(Cc2nnc(-c3cccc(Oc4cc5cc[nH]c5cn4)c3)[nH]2)c1] | 257 |
| 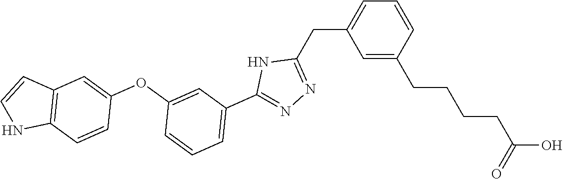 [SMILES: O=C(O)CCCCc1cccc(Cc2nnc(-c3cccc(Oc4ccc5[nH]ccc5c4)c3)[nH]2)c1] | 258 |
| 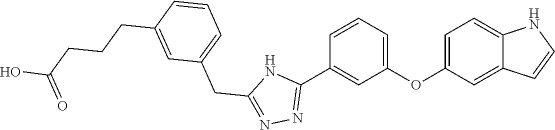 [SMILES: O=C(O)CCCc1cccc(Cc2nnc(-c3cccc(Oc4ccc5[nH]ccc5c4)c3)[nH]2)c1] | 259 |
| 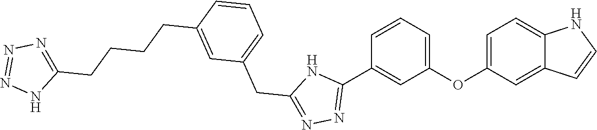 [SMILES: c1cc(CCCCc2nnn[nH]2)cc(Cc2nnc(-c3cccc(Oc4ccc5[nH]ccc5c4)c3)[nH]2)c1] | 260 |
| 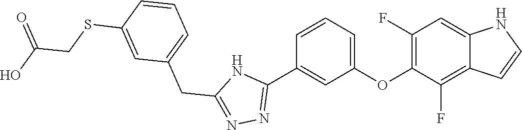 [SMILES: O=C(O)CSc1cccc(Cc2nnc(-c3cccc(Oc4c(F)cc5[nH]ccc5c4F)c3)[nH]2)c1] | 261 |
| 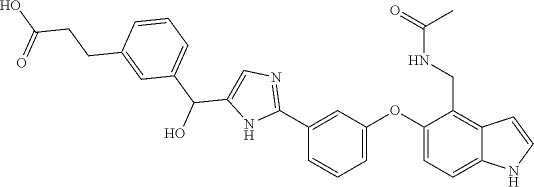 [SMILES: CC(=O)NCc1c(Oc2cccc(-c3ncc(C(O)c4cccc(CCC(=O)O)c4)[nH]3)c2)ccc2[nH]ccc12] | 262 |
| 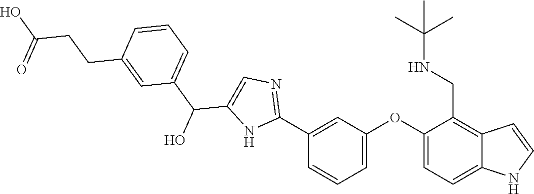 [SMILES: CC(C)(C)NCc1c(Oc2cccc(-c3ncc(C(O)c4cccc(CCC(=O)O)c4)[nH]3)c2)ccc2[nH]ccc12] | 263 |
| 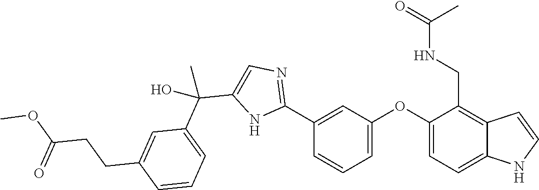 [SMILES: COC(=O)CCc1cccc(C(C)(O)c2cnc(-c3cccc(Oc4ccc5[nH]ccc5c4CNC(C)=O)c3)[nH]2)c1] | 264 |
| 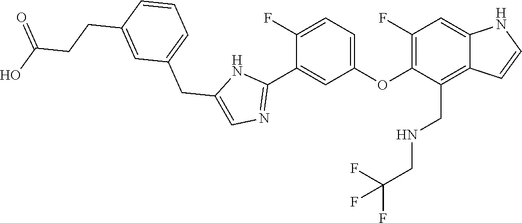 [SMILES: O=C(O)CCc1cccc(Cc2cnc(-c3cc(Oc4c(F)cc5[nH]ccc5c4CNCC(F)(F)F)ccc3F)[nH]2)c1] | 265 |
| 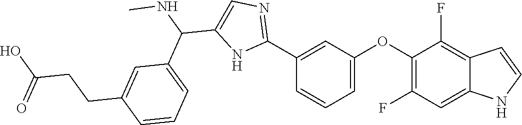 [SMILES: CNC(c1cccc(CCC(=O)O)c1)c1cnc(-c2cccc(Oc3c(F)cc4[nH]ccc4c3F)c2)[nH]1] | 266 |
| 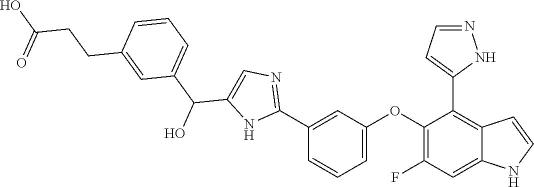 [SMILES: O=C(O)CCc1cccc(C(O)c2cnc(-c3cccc(Oc4c(F)cc5[nH]ccc5c4-c4ccn[nH]4)c3)[nH]2)c1] | 267 |
| 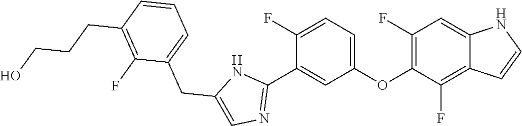 [SMILES: OCCCc1cccc(Cc2cnc(-c3cc(Oc4c(F)cc5[nH]ccc5c4F)ccc3F)[nH]2)c1F] | 268 |
| 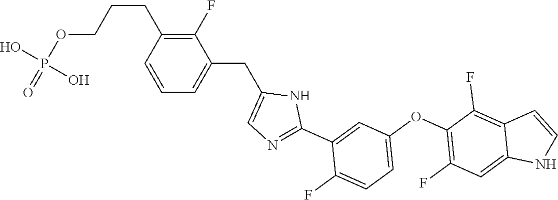 [SMILES: O=P(O)(O)OCCCc1cccc(Cc2cnc(-c3cc(Oc4c(F)cc5[nH]ccc5c4F)ccc3F)[nH]2)c1F] | 269 |
| 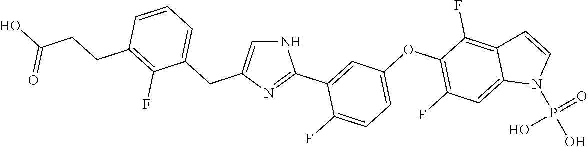 [SMILES: O=C(O)CCc1cccc(Cc2c[nH]c(-c3cc(Oc4c(F)cc5c(ccn5P(=O)(O)O)c4F)ccc3F)n2)c1F] | 270 |
| 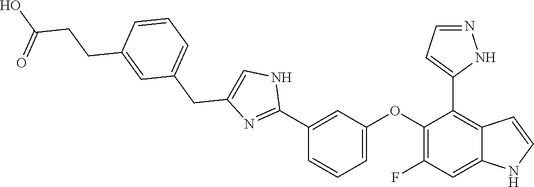 [SMILES: O=C(O)CCc1cccc(Cc2c[nH]c(-c3cccc(Oc4c(F)cc5[nH]ccc5c4-c4ccn[nH]4)c3)n2)c1] | 271 |
| 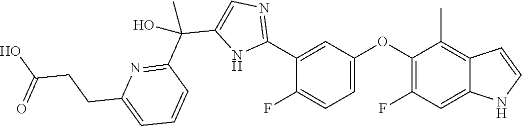 [SMILES: Cc1c(Oc2ccc(F)c(-c3ncc(C(C)(O)c4cccc(CCC(=O)O)n4)[nH]3)c2)c(F)cc2[nH]ccc12] | 272 |
| 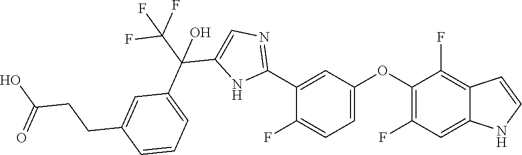 [SMILES: O=C(O)CCc1cccc(C(O)(c2cnc(-c3cc(Oc4c(F)cc5[nH]ccc5c4F)ccc3F)[nH]2)C(F)(F)F)c1] | 273 |
| 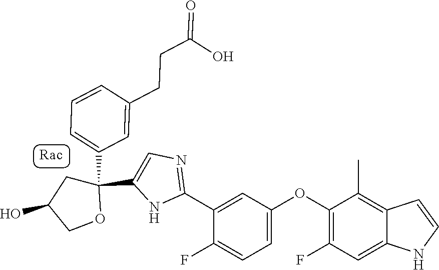 [SMILES: Cc1c(Oc2ccc(F)c(-c3ncc([C@]4(c5cccc(CCC(=O)O)c5)C[C@H](O)CO4)[nH]3)c2)c(F)cc2[nH]ccc12] | 274 |
| 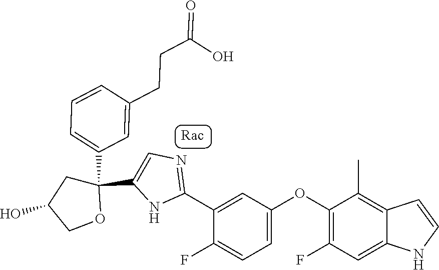 [SMILES: Cc1c(Oc2ccc(F)c(-c3ncc([C@]4(c5cccc(CCC(=O)O)c5)C[C@@H](O)CO4)[nH]3)c2)c(F)cc2[nH]ccc12] | 275 |
| 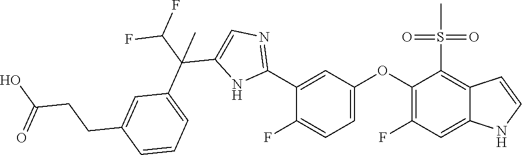 [SMILES: CC(c1cccc(CCC(=O)O)c1)(c1cnc(-c2cc(Oc3c(F)cc4[nH]ccc4c3S(C)(=O)=O)ccc2F)[nH]1)C(F)F] | 276 |
| 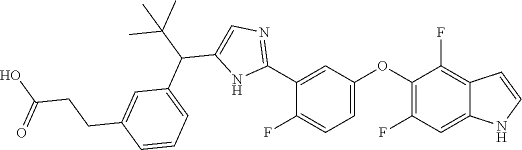 [SMILES: CC(C)(C)C(c1cccc(CCC(=O)O)c1)c1cnc(-c2cc(Oc3c(F)cc4[nH]ccc4c3F)ccc2F)[nH]1] | 277 |
| 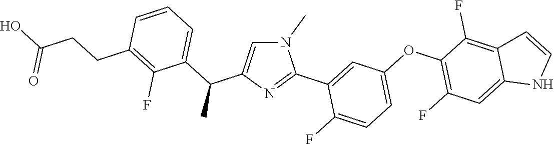 [SMILES: C[C@H](c1cn(C)c(-c2cc(Oc3c(F)cc4[nH]ccc4c3F)ccc2F)n1)c1cccc(CCC(=O)O)c1F] | 278A |
| 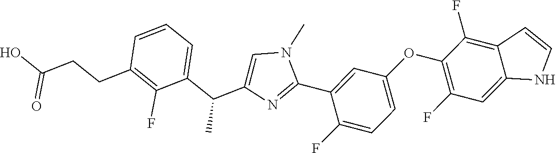 [SMILES: C[C@@H](c1cn(C)c(-c2cc(Oc3c(F)cc4[nH]ccc4c3F)ccc2F)n1)c1cccc(CCC(=O)O)c1F] | 278B |
| 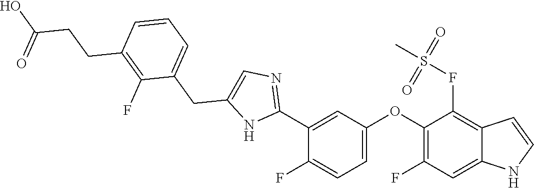 | 279 |
| 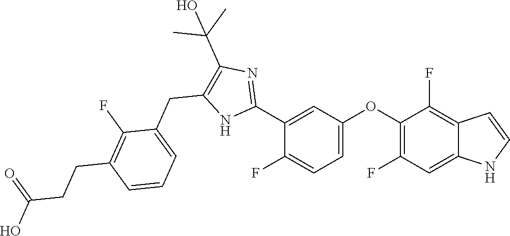 [SMILES: CC(C)(O)c1nc(-c2cc(Oc3c(F)cc4[nH]ccc4c3F)ccc2F)[nH]c1Cc1cccc(CCC(=O)O)c1F] | 280 |
| 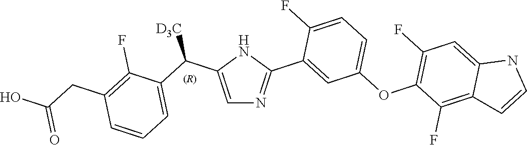 | 282A |
| 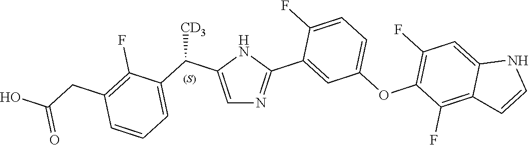 | 282B |

In some embodiments, the compound of Formula I is selected from the following compounds represented in Table 2 below:

TABLE 2

| Compound | Structure |
|---|---|
| 118 | 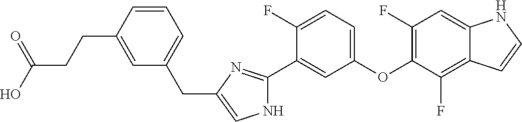 [SMILES: O=C(O)CCc1cccc(Cc2c[nH]c(-c3cc(Oc4c(F)cc5[nH]ccc5c4F)ccc3F)n2)c1] |
| 277 | 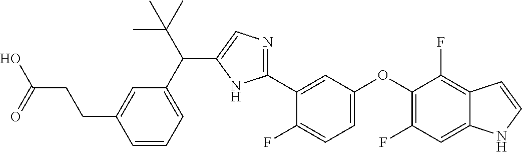 [SMILES: CC(C)(C)C(c1cccc(CCC(=O)O)c1)c1cnc(-c2cc(Oc3c(F)cc4[nH]ccc4c3F)ccc2F)[nH]1] |
| 123 | 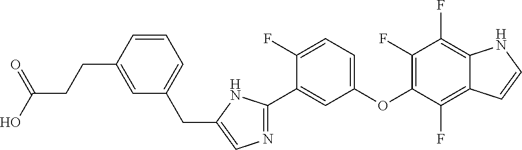 [SMILES: O=C(O)CCc1cccc(Cc2cnc(-c3cc(Oc4c(F)c(F)c5[nH]ccc5c4F)ccc3F)[nH]2)c1] |
| 64B | 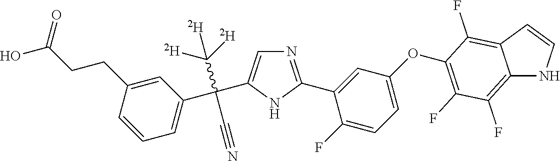 [SMILES: [2H]C([2H])([2H])C(C#N)(c1cccc(CCC(=O)O)c1)c1cnc(-c2cc(Oc3c(F)c(F)c4[nH]ccc4c3F)ccc2F)[nH]1] |
| 67B | 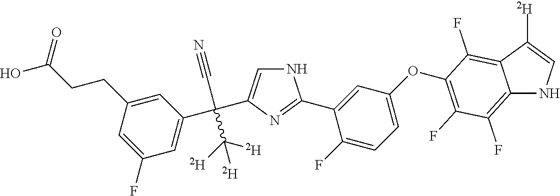 [SMILES: [2H]c1c[nH]c2c(F)c(F)c(Oc3ccc(F)c(-c4nc(C(C#N)(c5cc(F)cc(CCC(=O)O)c5)C([2H])([2H])[2H])c[nH]4)c3)c(F)c12] |
| 129A | 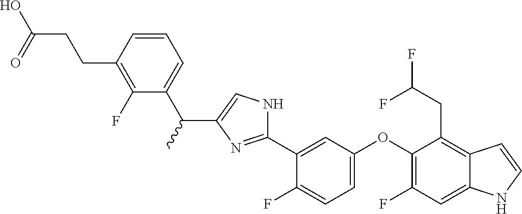 [SMILES: CC(c1c[nH]c(-c2cc(Oc3c(F)cc4[nH]ccc4c3CC(F)F)ccc2F)n1)c1cccc(CCC(=O)O)c1F] |
| 252A | 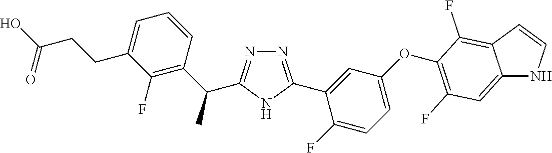 [SMILES: C[C@H](c1nnc(-c2cc(Oc3c(F)cc4[nH]ccc4c3F)ccc2F)[nH]1)c1cccc(CCC(=O)O)c1F] |
| 124 | 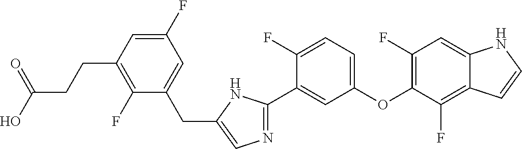 [SMILES: O=C(O)CCc1cc(F)cc(Cc2cnc(-c3cc(Oc4c(F)cc5[nH]ccc5c4F)ccc3F)[nH]2)c1F] |
| 280 | 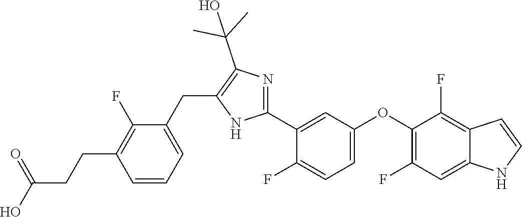 [SMILES: CC(C)(O)c1nc(-c2cc(Oc3c(F)cc4[nH]ccc4c3F)ccc2F)[nH]c1Cc1cccc(CCC(=O)O)c1F] |
| 278B | 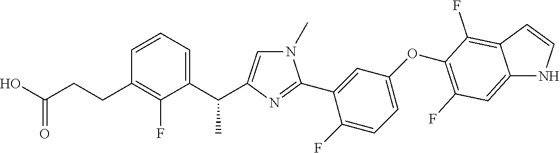 [SMILES: C[C@@H](c1cn(C)c(-c2cc(Oc3c(F)cc4[nH]ccc4c3F)ccc2F)n1)c1cccc(CCC(=O)O)c1F] |
| 125 | 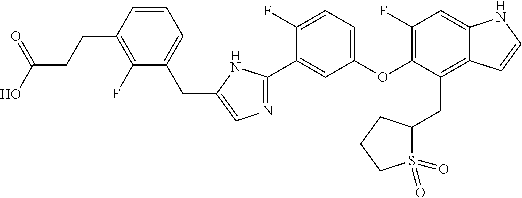 [SMILES: O=C(O)CCc1cccc(Cc2cnc(-c3cc(Oc4c(F)cc5[nH]ccc5c4CC4CCCS4(=O)=O)ccc3F)[nH]2)c1F] |
| 133B | 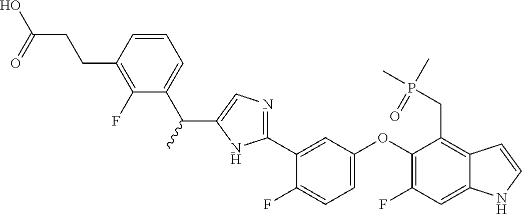 [SMILES: CC(c1cnc(-c2cc(Oc3c(F)cc4[nH]ccc4c3CP(C)(C)=O)ccc2F)[nH]1)c1cccc(CCC(=O)O)c1F] |
| 119 | 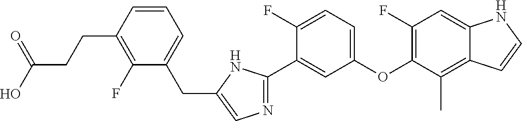 [SMILES: Cc1c(Oc2ccc(F)c(-c3ncc(Cc4cccc(CCC(=O)O)c4F)[nH]3)c2)c(F)cc2[nH]ccc12] |
| 121 | 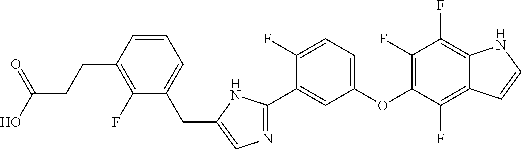 [SMILES: O=C(O)CCc1cccc(Cc2cnc(-c3cc(Oc4c(F)c(F)c5[nH]ccc5c4F)ccc3F)[nH]2)c1F] |
| 128B | 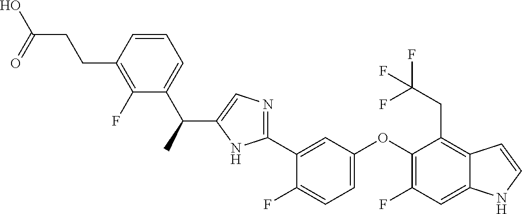 [SMILES: C[C@H](c1cnc(-c2cc(Oc3c(F)cc4[nH]ccc4c3CC(F)(F)F)ccc2F)[nH]1)c1cccc(CCC(=O)O)c1F] |
| 43A | 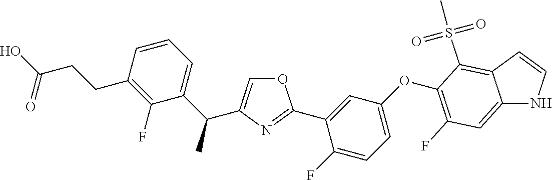 [SMILES: C[C@H](c1coc(-c2cc(Oc3c(F)cc4[nH]ccc4c3S(C)(=O)=O)ccc2F)n1)c1cccc(CCC(=O)O)c1F] |
| 279 | 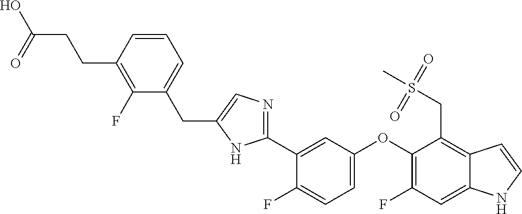 [SMILES: CS(=O)(=O)Cc1c(Oc2ccc(F)c(-c3ncc(Cc4cccc(CCC(=O)O)c4F)[nH]3)c2)c(F)cc2[nH]ccc12] |
| 131A | 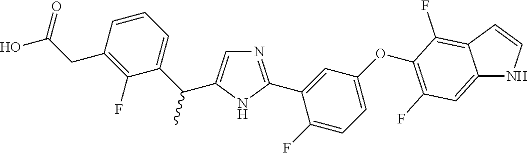 [SMILES: CC(c1cnc(-c2cc(Oc3c(F)cc4[nH]ccc4c3F)ccc2F)[nH]1)c1cccc(CC(=O)O)c1F] |
| 41B | 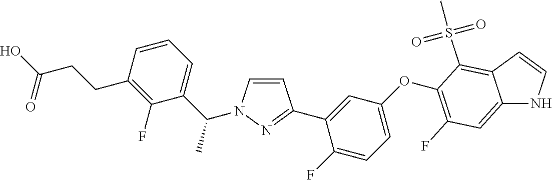 [SMILES: C[C@H](c1cccc(CCC(=O)O)c1F)n1ccc(-c2cc(Oc3c(F)cc4[nH]ccc4c3S(C)(=O)=O)ccc2F)n1] |
| 132B | 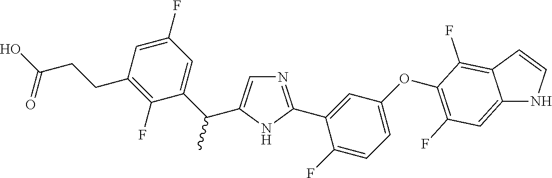 [SMILES: CC(c1cnc(-c2cc(Oc3c(F)cc4[nH]ccc4c3F)ccc2F)[nH]1)c1cc(F)cc(CCC(=O)O)c1F] |
| 136C | 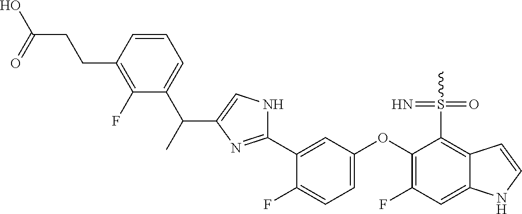 [SMILES: CC(c1c[nH]c(-c2cc(Oc3c(F)cc4[nH]ccc4c3S(C)(=N)=O)ccc2F)n1)c1cccc(CCC(=O)O)c1F] |
| 137D | 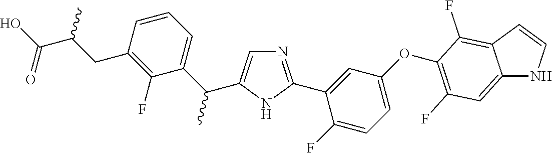 |
| 84 | 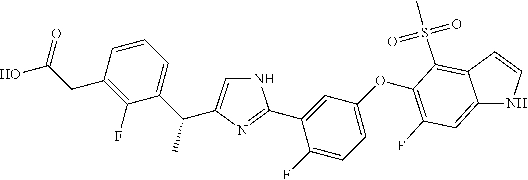 [SMILES: C[C@@H](c1c[nH]c(-c2cc(Oc3c(F)cc4[nH]ccc4c3S(C)(=O)=O)ccc2F)n1)c1cccc(CC(=O)O)c1F] |
| 126 | 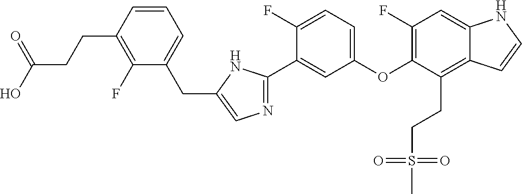 [SMILES: CS(=O)(=O)CCc1c(Oc2ccc(F)c(-c3ncc(Cc4cccc(CCC(=O)O)c4F)[nH]3)c2)c(F)cc2[nH]ccc12] |
| 134B | 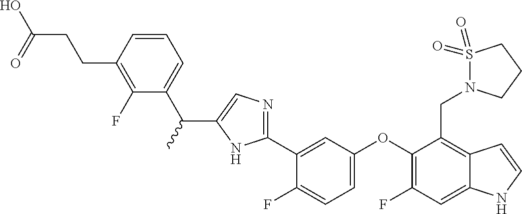 [SMILES: CC(c1cnc(-c2cc(Oc3c(F)cc4[nH]ccc4c3CN3CCCS3(=O)=O)ccc2F)[nH]1)c1cccc(CCC(=O)O)c1F] |
| 32 | 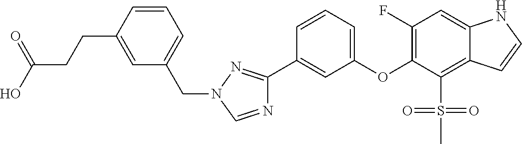 [SMILES: CS(=O)(=O)c1c(Oc2cccc(-c3ncn(Cc4cccc(CCC(=O)O)c4)n3)c2)c(F)cc2[nH]ccc12] |
| 272 | 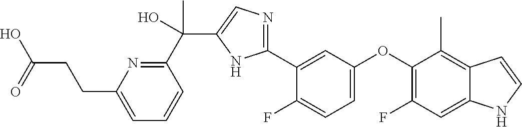 [SMILES: Cc1c(Oc2ccc(F)c(-c3ncc(C(C)(O)c4cccc(CCC(=O)O)n4)[nH]3)c2)c(F)cc2[nH]ccc12] |
| 273 | 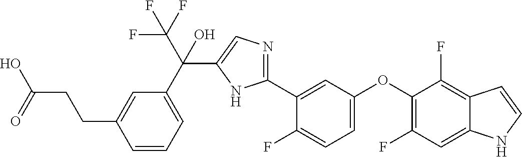 [SMILES: O=C(O)CCc1cccc(C(O)(c2cnc(-c3cc(Oc4c(F)cc5[nH]ccc5c4F)ccc3F)[nH]2)C(F)(F)F)c1] |
| 194 | 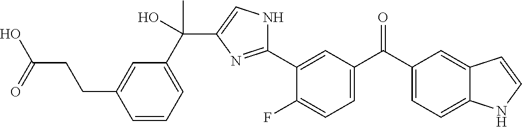 [SMILES: CC(O)(c1cccc(CCC(=O)O)c1)c1c[nH]c(-c2cc(C(=O)c3ccc4[nH]ccc4c3)ccc2F)n1] |
| 167 | 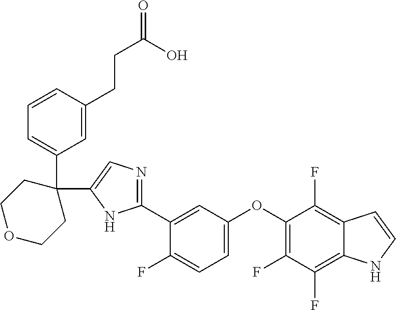 [SMILES: O=C(O)CCc1cccc(C2(c3cnc(-c4cc(Oc5c(F)c(F)c6[nH]ccc6c5F)ccc4F)[nH]3)CCOCC2)c1] |
| 171 | 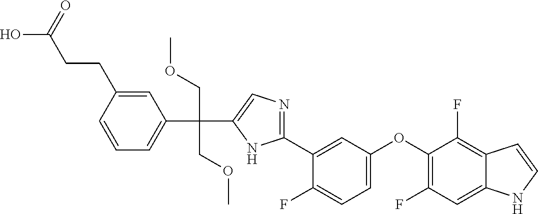 [SMILES: COCC(COC)(c1cccc(CCC(=O)O)c1)c1cnc(-c2cc(Oc3c(F)cc4[nH]ccc4c3F)ccc2F)[nH]1] |
| 100 | 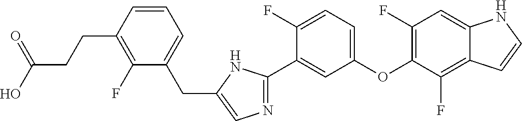 [SMILES: O=C(O)CCc1cccc(Cc2cnc(-c3cc(Oc4c(F)cc5[nH]ccc5c4F)ccc3F)[nH]2)c1F] |
| 266 | 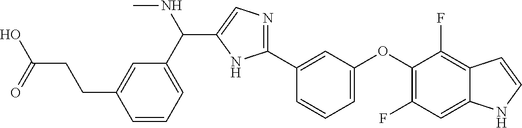 [SMILES: CNC(c1cccc(CCC(=O)O)c1)c1cnc(-c2cccc(Oc3c(F)cc4[nH]ccc4c3F)c2)[nH]1] |
| 140 | 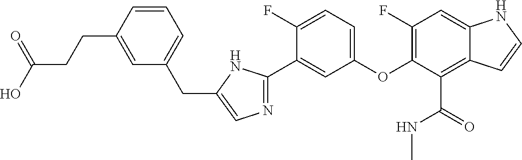 [SMILES: CNC(=O)c1c(Oc2ccc(F)c(-c3ncc(Cc4cccc(CCC(=O)O)c4)[nH]3)c2)c(F)cc2[nH]ccc12] |
| 97 | 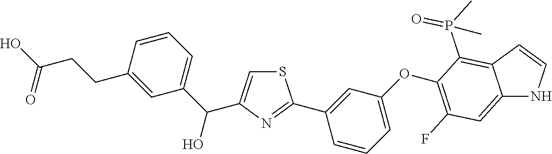 [SMILES: CP(C)(=O)c1c(Oc2cccc(-c3nc(C(O)c4cccc(CCC(=O)O)c4)cs3)c2)c(F)cc2[nH]ccc12] |
| 274 | 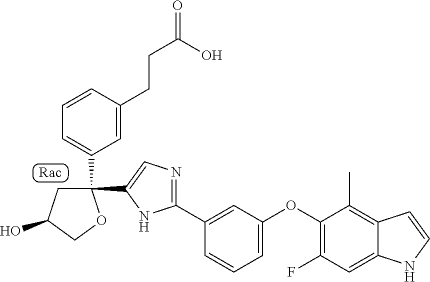 [SMILES: Cc1c(Oc2cccc(-c3ncc([C@]4(c5cccc(CCC(=O)O)c5)C[C@H](O)CO4)[nH]3)c2)c(F)cc2[nH]ccc12] |
| 77 | 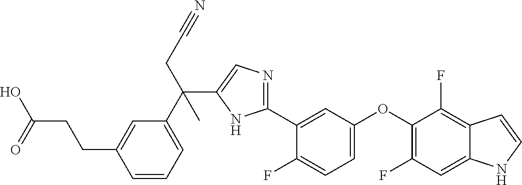 [SMILES: CC(CC#N)(c1cccc(CCC(=O)O)c1)c1cnc(-c2cc(Oc3c(F)cc4[nH]ccc4c3F)ccc2F)[nH]1] |
| 206 | 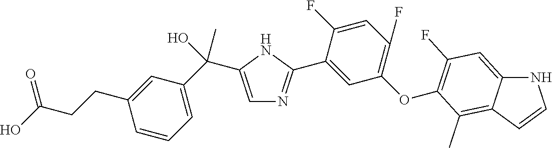 [SMILES: Cc1c(Oc2cc(-c3ncc(C(C)(O)c4cccc(CCC(=O)O)c4)[nH]3)c(F)cc2F)c(F)cc2[nH]ccc12] |
| 45 | 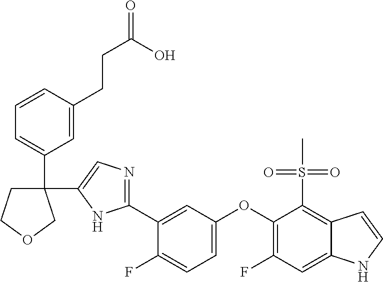 [SMILES: CS(=O)(=O)c1c(Oc2ccc(F)c(-c3ncc(C4(c5cccc(CCC(=O)O)c5)CCOC4)[nH]3)c2)c(F)cc2[nH]ccc12] |
| 170 | 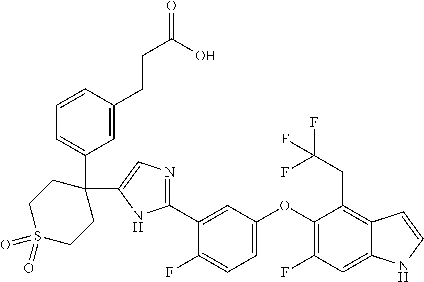 [SMILES: O=C(O)CCc1cccc(C2(c3cnc(-c4cc(Oc5c(F)cc6[nH]ccc6c5CC(F)(F)F)ccc4F)[nH]3)CCS(=O)(=O)CC2)c1] |
| 276 | 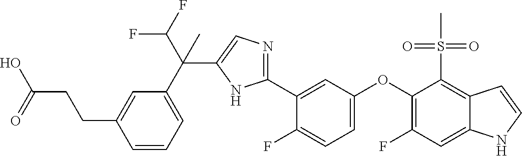 [SMILES: CC(c1cccc(CCC(=O)O)c1)(c1cnc(-c2cc(Oc3c(F)cc4[nH]ccc4c3S(C)(=O)=O)ccc2F)[nH]1)C(F)F] |
| 270 | 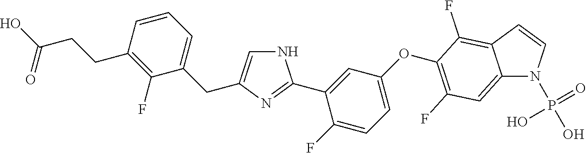 [SMILES: O=C(O)CCc1cccc(Cc2c[nH]c(-c3cc(Oc4c(F)cc5c(ccn5P(=O)(O)O)c4F)ccc3F)n2)c1F] |
| 130B | 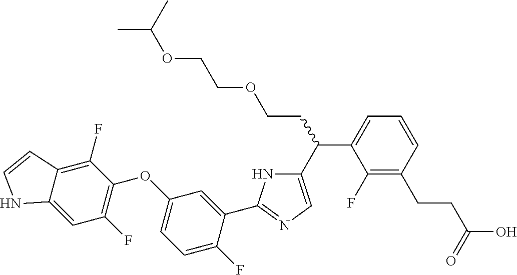 [SMILES: CC(C)OCCOCCC(c1cnc(-c2cc(Oc3c(F)cc4[nH]ccc4c3F)ccc2F)[nH]1)c1cccc(CCC(=O)O)c1F] |
| 80 | 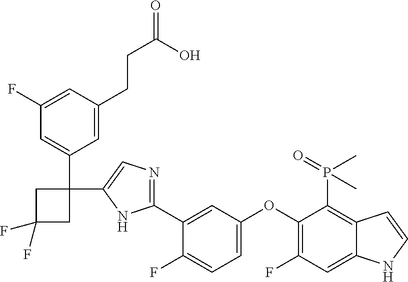 [SMILES: CP(C)(=O)c1c(Oc2ccc(F)c(-c3ncc(C4(c5cc(F)cc(CCC(=O)O)c5)CC(F)(F)C4)[nH]3)c2)c(F)cc2[nH]ccc12] |
| 75 | 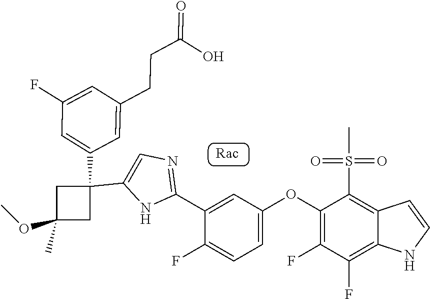 [SMILES: CO[C@]1(C)C[C@](c2cc(F)cc(CCC(=O)O)c2)(c2cnc(-c3cc(Oc4c(F)c(F)c5[nH]ccc5c4S(C)(=O)=O)ccc3F)[nH]2)C1] |
| 135A | 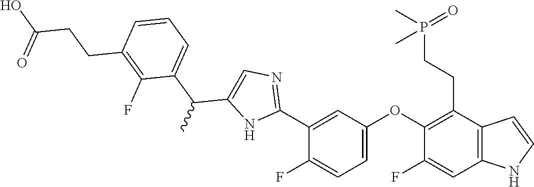 [SMILES: CC(c1cnc(-c2cc(Oc3c(F)cc4[nH]ccc4c3CCP(C)(C)=O)ccc2F)[nH]1)c1cccc(CCC(=O)O)c1F] |
| 269 | 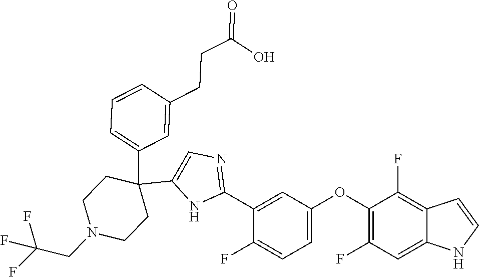 [SMILES: O=C(O)CCc1cccc(C2(c3cnc(-c4cc(Oc5c(F)cc6[nH]ccc6c5F)ccc4F)[nH]3)CCN(CC(F)(F)F)CC2)c1] |
| 45 | 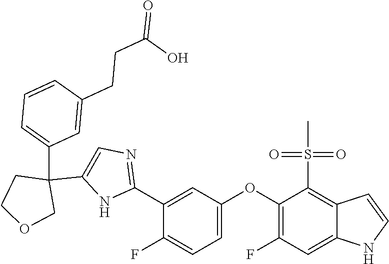 [SMILES: CS(=O)(=O)c1c(Oc2ccc(F)c(-c3ncc(C4(c5cccc(CCC(=O)O)c5)CCOC4)[nH]3)c2)c(F)cc2[nH]ccc12] |
| 180 | 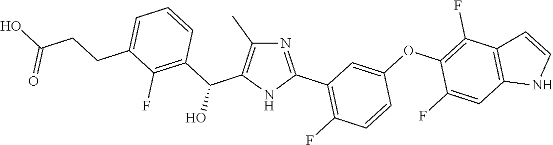 [SMILES: Cc1nc(-c2cc(Oc3c(F)cc4[nH]ccc4c3F)ccc2F)[nH]c1[C@H](O)c1cccc(CCC(=O)O)c1F] |
| 23B | 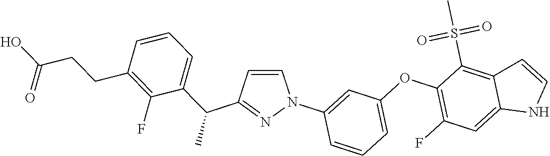 [SMILES: C[C@@H](c1ccn(-c2cccc(Oc3c(F)cc4[nH]ccc4c3S(C)(=O)=O)c2)n1)c1cccc(CCC(=O)O)c1F] |
| 139F | 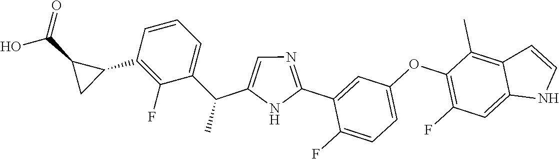 [SMILES: Cc1c(Oc2ccc(F)c(-c3ncc([C@H](C)c4cccc([C@@H]5C[C@H]5C(=O)O)c4F)[nH]3)c2)c(F)cc2[nH]ccc12] |
| 269 | 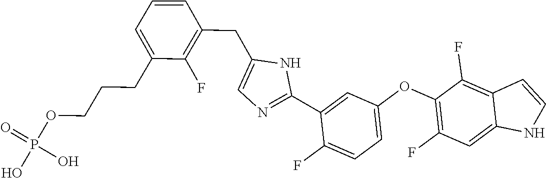 [SMILES: O=P(O)(O)OCCCc1cccc(Cc2cnc(-c3cc(Oc4c(F)cc5[nH]ccc5c4F)ccc3F)[nH]2)c1F] |

In some embodiments, the compound of Formula I is selected from the following compounds represented in Table 3 below:

TABLE 3

| Compound | Structure |
|---|---|
| 118 | 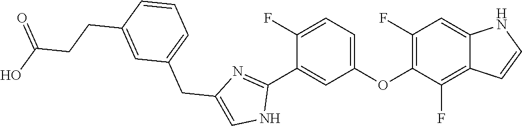 [SMILES: O=C(O)CCc1cccc(Cc2c[nH]c(-c3cc(Oc4c(F)cc5[nH]ccc5c4F)ccc3F)n2)c1] |
| 123 | 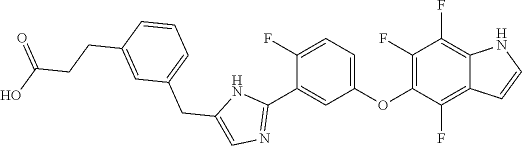 [SMILES: O=C(O)CCc1cccc(Cc2cnc(-c3cc(Oc4c(F)c(F)c5[nH]ccc5c4F)ccc3F)[nH]2)c1] |
| 64B | 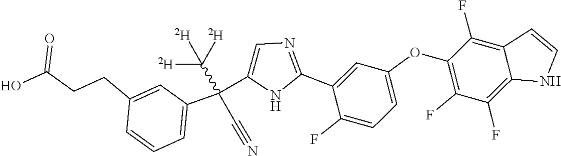 [SMILES: [2H]C([2H])([2H])C(C#N)(c1cccc(CCC(=O)O)c1)c1cnc(-c2cc(Oc3c(F)c(F)c4[nH]ccc4c3F)ccc2F)[nH]1] |
| 67B | 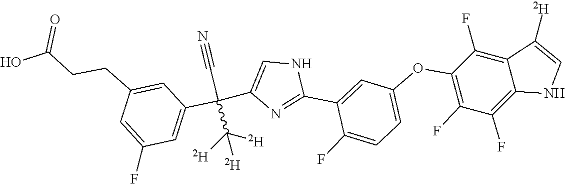 [SMILES: [2H]c1c[nH]c2c(F)c(F)c(Oc3ccc(F)c(-c4nc(C(C#N)(c5cc(F)cc(CCC(=O)O)c5)C([2H])([2H])[2H])c[nH]4)c3)c(F)c12] |
| 129A | 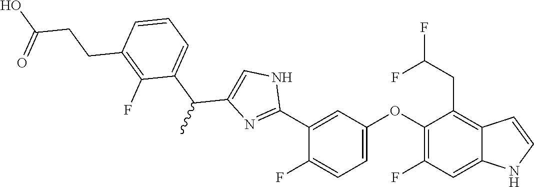 [SMILES: CC(c1c[nH]c(-c2cc(Oc3c(F)cc4[nH]ccc4c3CC(F)F)ccc2F)n1)c1cccc(CCC(=O)O)c1F] |
| 252A | 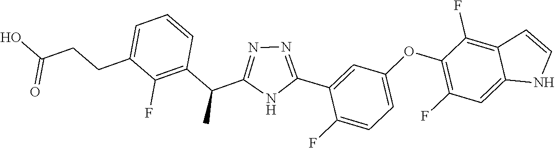 [SMILES: C[C@H](c1nnc(-c2cc(Oc3c(F)cc4[nH]ccc4c3F)ccc2F)[nH]1)c1cccc(CCC(=O)O)c1F] |
| 124 | 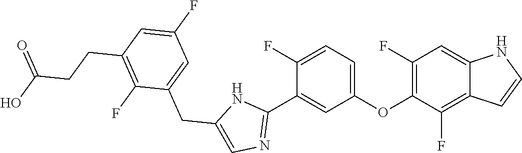 [SMILES: O=C(O)CCc1cc(F)cc(Cc2cnc(-c3cc(Oc4c(F)cc5[nH]ccc5c4F)ccc3F)[nH]2)c1F] |
| 280 | 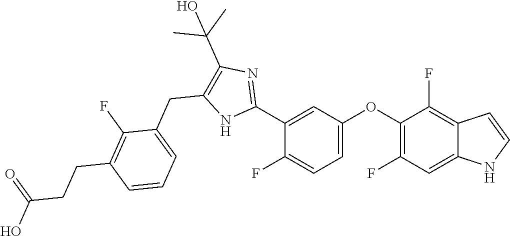 [SMILES: CC(C)(O)c1nc(-c2cc(Oc3c(F)cc4[nH]ccc4c3F)ccc2F)[nH]c1Cc1cccc(CCC(=O)O)c1F] |
| 278B | 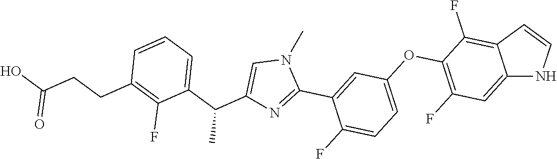 [SMILES: C[C@@H](c1cn(C)c(-c2cc(Oc3c(F)cc4[nH]ccc4c3F)ccc2F)n1)c1cccc(CCC(=O)O)c1F] |
| 125 | 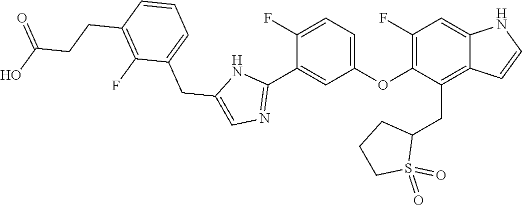 [SMILES: O=C(O)CCc1cccc(Cc2cnc(-c3cc(Oc4c(F)cc5[nH]ccc5c4CC4CCCS4(=O)=O)ccc3F)[nH]2)c1F] |
| 277 | 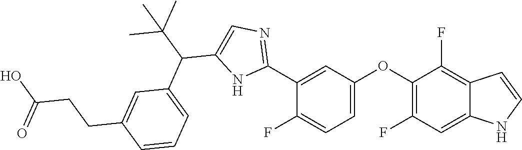 [SMILES: CC(C)(C)C(c1cccc(CCC(=O)O)c1)c1cnc(-c2cc(Oc3c(F)cc4[nH]ccc4c3F)ccc2F)[nH]1] |
| 119 | 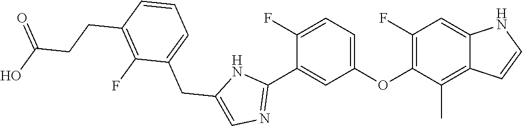 [SMILES: Cc1c(Oc2ccc(F)c(-c3ncc(Cc4cccc(CCC(=O)O)c4F)[nH]3)c2)c(F)cc2[nH]ccc12] |
| 121 | 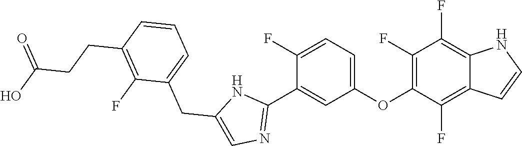 [SMILES: O=C(O)CCc1cccc(Cc2cnc(-c3cc(Oc4c(F)c(F)c5[nH]ccc5c4F)ccc3F)[nH]2)c1F] |
| 128B | 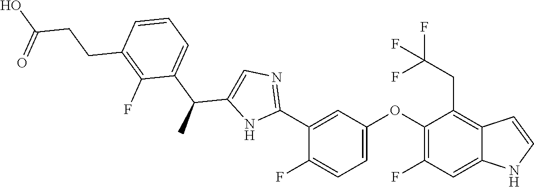 [SMILES: C[C@H](c1cnc(-c2cc(Oc3c(F)cc4[nH]ccc4c3CC(F)(F)F)ccc2F)[nH]1)c1cccc(CCC(=O)O)c1F] |
| 43A | 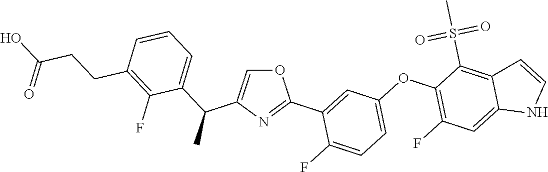 [SMILES: C[C@H](c1coc(-c2cc(Oc3c(F)cc4[nH]ccc4c3S(C)(=O)=O)ccc2F)n1)c1cccc(CCC(=O)O)c1F] |
| 279 | 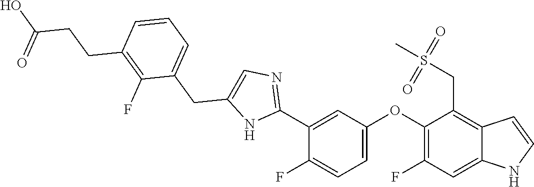 [SMILES: CS(=O)(=O)Cc1c(Oc2ccc(F)c(-c3ncc(Cc4cccc(CCC(=O)O)c4F)[nH]3)c2)c(F)cc2[nH]ccc12] |
| 131A | 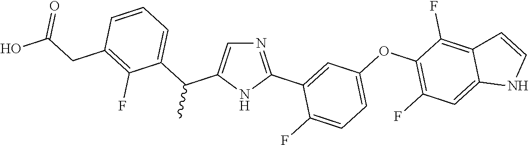 [SMILES: CC(c1cnc(-c2cc(Oc3c(F)cc4[nH]ccc4c3F)ccc2F)[nH]1)c1cccc(CC(=O)O)c1F] |
| 41B | 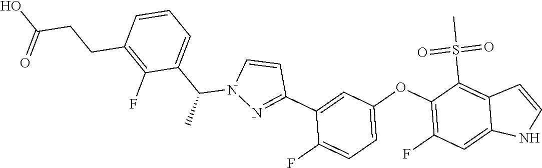 [SMILES: C[C@H](c1cccc(CCC(=O)O)c1F)n1ccc(-c2cc(Oc3c(F)cc4[nH]ccc4c3S(C)(=O)=O)ccc2F)n1] |
| 132B | 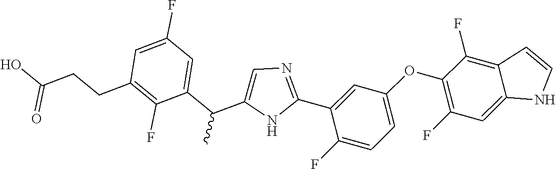 [SMILES: CC(c1cnc(-c2cc(Oc3c(F)cc4[nH]ccc4c3F)ccc2F)[nH]1)c1cc(F)cc(CCC(=O)O)c1F] |
| 136C | 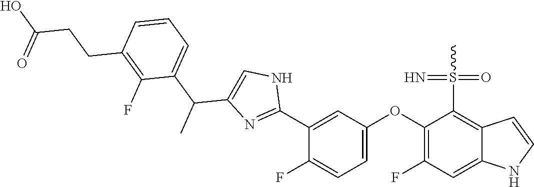 [SMILES: CC(c1c[nH]c(-c2cc(Oc3c(F)cc4[nH]ccc4c3S(C)(=N)=O)ccc2F)n1)c1cccc(CCC(=O)O)c1F] |
| 133B | 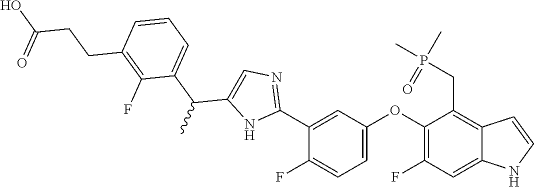 [SMILES: CC(c1cnc(-c2cc(Oc3c(F)cc4[nH]ccc4c3CP(C)(C)=O)ccc2F)[nH]1)c1cccc(CCC(=O)O)c1F] |
| 84 | 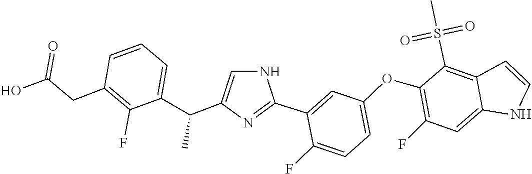 [SMILES: C[C@@H](c1c[nH]c(-c2cc(Oc3c(F)cc4[nH]ccc4c3S(C)(=O)=O)ccc2F)n1)c1cccc(CC(=O)O)c1F] |
| 126 | 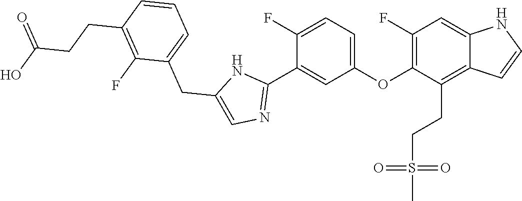 [SMILES: CS(=O)(=O)CCc1c(Oc2ccc(F)c(-c3ncc(Cc4cccc(CCC(=O)O)c4F)[nH]3)c2)c(F)cc2[nH]ccc12] |
| 270 | 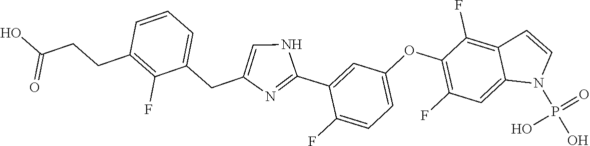 [SMILES: O=C(O)CCc1cccc(Cc2c[nH]c(-c3cc(Oc4c(F)cc5c(ccn5P(=O)(O)O)c4F)ccc3F)n2)c1F] |
| 137D | 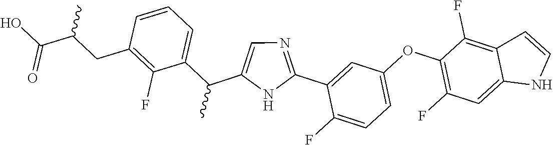 |
| 100 | 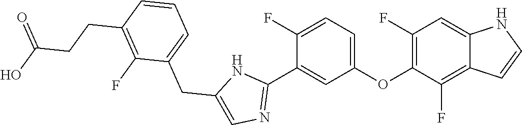 [SMILES: O=C(O)CCc1cccc(Cc2cnc(-c3cc(Oc4c(F)cc5[nH]ccc5c4F)ccc3F)[nH]2)c1F] |
| 134B | 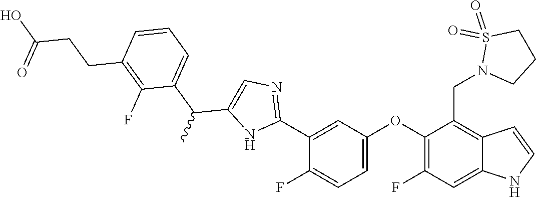 [SMILES: CC(c1cnc(-c2cc(Oc3c(F)cc4[nH]ccc4c3CN3CCCS3(=O)=O)ccc2F)[nH]1)c1cccc(CCC(=O)O)c1F] |
| 269 | 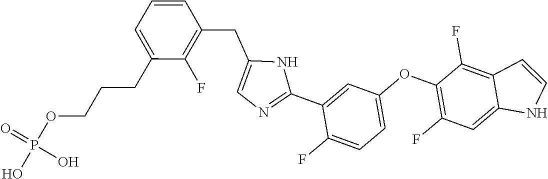 [SMILES: O=P(O)(O)OCCCc1cccc(Cc2cnc(-c3cc(Oc4c(F)cc5[nH]ccc5c4F)ccc3F)[nH]2)c1F] |

In some embodiments, the compound of Formula I is selected from the following compounds represented in Table 4 below:

TABLE 4

| Compound | Structure |
|---|---|
| 118 | 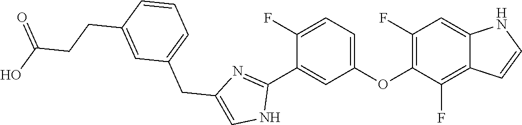 [SMILES: O=C(O)CCc1cccc(Cc2c[nH]c(-c3cc(Oc4c(F)cc5[nH]ccc5c4F)ccc3F)n2)c1] |
| 123 | 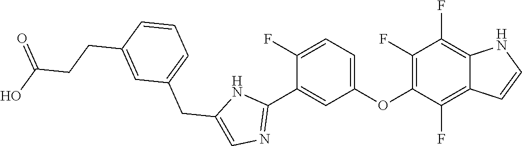 [SMILES: O=C(O)CCc1cccc(Cc2cnc(-c3cc(Oc4c(F)c(F)c5[nH]ccc5c4F)ccc3F)[nH]2)c1] |
| 124 | 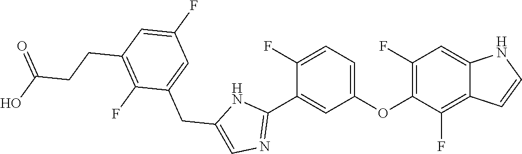 [SMILES: O=C(O)CCc1cc(F)cc(Cc2cnc(-c3cc(Oc4c(F)cc5[nH]ccc5c4F)ccc3F)[nH]2)c1F] |
| 41B | 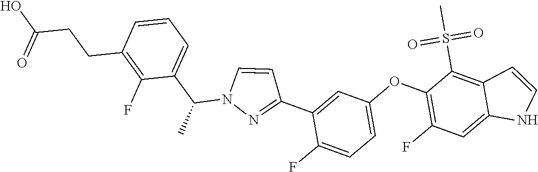 [SMILES: C[C@H](c1cccc(CCC(=O)O)c1F)n1ccc(-c2cc(Oc3c(F)cc4[nH]ccc4c3S(C)(=O)=O)ccc2F)n1] |
| 84 | 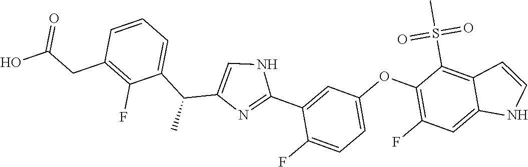 [SMILES: C[C@@H](c1c[nH]c(-c2cc(Oc3c(F)cc4[nH]ccc4c3S(C)(=O)=O)ccc2F)n1)c1cccc(CC(=O)O)c1F] |
| 121 | 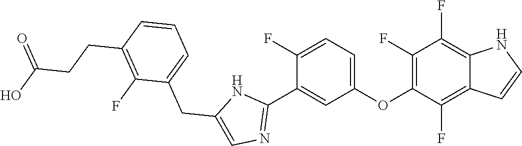 [SMILES: O=C(O)CCc1cccc(Cc2cnc(-c3cc(Oc4c(F)c(F)c5[nH]ccc5c4F)ccc3F)[nH]2)c1F] |
| 43A | 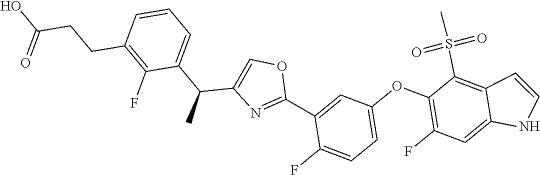 [SMILES: C[C@H](c1coc(-c2cc(Oc3c(F)cc4[nH]ccc4c3S(C)(=O)=O)ccc2F)n1)c1cccc(CCC(=O)O)c1F] |
| 131A | 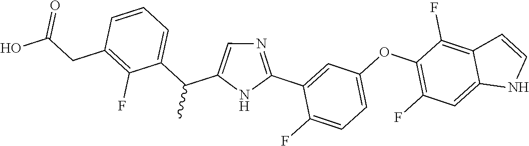 [SMILES: CC(c1cnc(-c2cc(Oc3c(F)cc4[nH]ccc4c3F)ccc2F)[nH]1)c1cccc(CC(=O)O)c1F] |
| 132B | 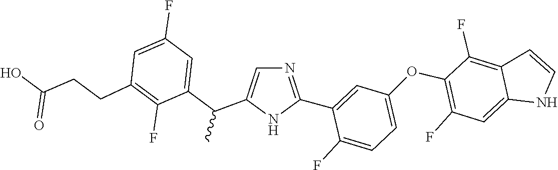 [SMILES: CC(c1cnc(-c2cc(Oc3c(F)cc4[nH]ccc4c3F)ccc2F)[nH]1)c1cc(F)cc(CCC(=O)O)c1F] |
| 100 | 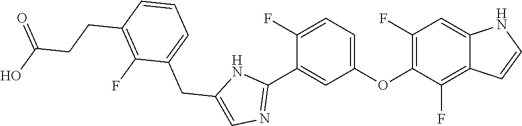 [SMILES: O=C(O)CCc1cccc(Cc2cnc(-c3cc(Oc4c(F)cc5[nH]ccc5c4F)ccc3F)[nH]2)c1F] |

In certain embodiments, the present invention provides a pharmaceutical preparation suitable for use in a subject, comprising any of the compounds shown above (e.g., a compound of the invention, such as a compound of formula I, and one or more pharmaceutically acceptable excipients. In certain embodiments, the pharmaceutical preparations may be for use in treating or preventing cystic fibrosis.

Any of the disclosed compounds may be used in the manufacture of medicaments for the treatment of any diseases or conditions disclosed herein.

### Definitions

Unless defined otherwise, all technical and scientific terms used herein have the meaning commonly understood by a person skilled in the art of the present disclosure. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

In this disclosure, “comprises,” “comprising,” “containing” and “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

Unless specifically stated or obvious from context, as used herein, the term “or” is understood to be inclusive. Unless specifically stated or obvious from context, as used herein, the terms “a”, “an”, and “the” are understood to be singular or plural.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group, preferably a lower alkyl group, having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “alkenyl”, as used herein, refers to a straight chained or branched aliphatic group containing at least one double bond. Typically, an alkenyl group has from 2 to about 20 carbon atoms, preferably from 2 to about 10, more preferably from 2-6 or 2-4. unless otherwise defined. The term “alkenyl” is intended to include both “unsubstituted alkenyls” and “substituted alkenyls”, the latter of which refers to alkenyl moieties having substituents replacing a hydrogen on one or more carbons of the alkenyl group. Such substituents may occur on one or more carbons that are included or not included in one or more double bonds. Moreover, such substituents include all those contemplated for alkyl groups, as discussed below, except where stability is prohibitive. For example, substitution of alkenyl groups by one or more alkyl, carbocyclyl, aryl, heterocyclyl, or heteroaryl groups is contemplated.

An “alkyl” group or “alkane” is a straight chained or branched non-aromatic hydrocarbon which is completely saturated. Typically, a straight chained or branched alkyl group has from 1 to about 20 carbon atoms, preferably from 1 to about 10, more preferably from 1-6 or 1-4. unless otherwise defined. Examples of straight chained and branched alkyl groups include methyl, ethyl, n-propyl, iso-propyl, n-butyl, sec-butyl, tert-butyl, pentyl, hexyl, pentyl and octyl. A C₁-C₆ straight chained or branched alkyl group is also referred to as a “lower alkyl” group.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents, if not otherwise specified, can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxy, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN, and the like.

The term “C_{x-y}” when used in conjunction with a chemical moiety, such as, acyl, acyloxy, alkyl, alkenyl, alkynyl, or alkoxy is meant to include groups that contain from x to y carbons in the chain. For example, the term “C_{x-y}alkyl” refers to substituted or unsubstituted saturated hydrocarbon groups, including straight-chain alkyl and branched-chain alkyl groups that contain from x to y carbons in the chain, including haloalkyl groups such as trifluoromethyl and 2,2,2-tirfluoroethyl, etc. C₀ alkyl indicates a hydrogen where the group is in a terminal position, a bond if internal. The terms “C_{2-y}alkenyl” and “C_{2-y}alkynyl” refer to substituted or unsubstituted unsaturated aliphatic groups analogous in length and possible substitution to the alkyls described above, but that contain at least one double or triple bond respectively.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “haloalkyl”, as used herein, refers to an alkyl group in which at least one hydrogen has been replaced with a halogen, such as fluoro, chloro, bromo, or iodo.

Exemplary haloalkyl groups include trifluoromethyl, difluoromethyl, fluoromethyl, 2-fluoroethyl, 2,2-difluoroethyl, and 2,2,2-trifluoroethyl.

The term “alkynyl”, as used herein, refers to a straight chained or branched aliphatic group containing at least one triple bond. Typically, an alkenyl group has from 2 to about 20 carbon atoms, preferably from 2 to about 10, more preferably from 2-6 or 2-4. unless otherwise defined. The term “alkynyl” is intended to include both “unsubstituted alkynyls” and “substituted alkynyls”, the latter of which refers to alkynyl moieties having substituents replacing a hydrogen on one or more carbons of the alkynyl group. Such substituents may occur on one or more carbons that are included or not included in one or more triple bonds.

Moreover, such substituents include all those contemplated for alkyl groups, as discussed above, except where stability is prohibitive. For example, substitution of alkynyl groups by one or more alkyl, carbocyclyl, aryl, heterocyclyl, or heteroaryl groups is contemplated.

The term “amide”, as used herein, refers to a group

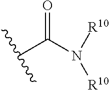

wherein each R¹⁰ independently represents a hydrogen or hydrocarbyl group, or two R¹⁰ are taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

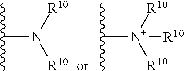

wherein each R¹⁰ independently represents a hydrogen or a hydrocarbyl group, or two R¹⁰ are taken together with the N atom to which they are attached complete a heterocycle having from 4 to 8 atoms in the ring structure. The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably, the ring is a 6- to 10-membered ring, such as a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

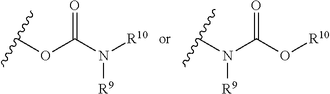

wherein R⁹ and R¹⁰ independently represent hydrogen or a hydrocarbyl group, such as an alkyl group, or R⁹ and R¹⁰ taken together with the intervening atom(s) complete a heterocycle having from 4 to 8 atoms in the ring structure.

The terms “carbocycle”, and “carbocyclic”, as used herein, refers to a saturated or unsaturated ring in which each atom of the ring is carbon. The term carbocycle includes both aromatic carbocycles and non-aromatic carbocycles. Non-aromatic carbocycles include both cycloalkane rings, in which all carbon atoms are saturated, and cycloalkene rings, which contain at least one double bond.

The term “carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring.

Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

A “cycloalkyl” group is a cyclic hydrocarbon which is completely saturated. “Cycloalkyl” includes monocyclic and bicyclic rings. Typically, a monocyclic cycloalkyl group has from 3 to about 10 carbon atoms, more typically 3 to 9 carbon atoms unless otherwise defined. The second ring of a bicyclic cycloalkyl may be selected from saturated, unsaturated and aromatic rings. Cycloalkyl includes bicyclic molecules in which one, two or three or more atoms are shared between the two rings. The term “fused cycloalkyl” refers to a bicyclic cycloalkyl in which each of the rings shares two adjacent atoms with the other ring. The second ring of a fused bicyclic cycloalkyl may be selected from saturated, unsaturated and aromatic rings.

A “cycloalkenyl” group is a cyclic hydrocarbon containing one or more double bonds. The cycloalkenyl ring may have 3 to 10 carbon atoms, such as 4 to 9 carbon atoms. As such, cycloalkenyl groups can be monocyclic or multicyclic. Individual rings of such multicyclic cycloalkenyl groups can have different connectivities, e.g., fused, bridged, spiro, etc. in addition to covalent bond substitution. Exemplary cycloalkenyl groups include cyclopropenyl, cyclobutenyl, cyclopentyl, cyclohexenyl, cycloheptenyl, 1,3-cyclohexadienyl, 1,4-cyclohexadienyl and 1,5-cyclooctadienyl.

Exemplary cycloalkyl groups include cyclopropyl, cyclobutyl, cyclopentyl, cyclohexyl, norbornanyl, bicyclo[3.2.1]octanyl, octahydro-pentalenyl, spiro[4.5]decanyl, cyclopropyl, and adamantyl.

The term “carbocyclylalkyl”, as used herein, refers to an alkyl group substituted with a carbocycle group.

The term “carbonate” is art-recognized and refers to a group —OCO₂—R¹⁰, wherein R¹⁰ represents a hydrocarbyl group.

The term “carboxy”, as used herein, refers to a group represented by the formula —CO₂H.

The term “ester”, as used herein, refers to a group —C(O)OR¹⁰ wherein R¹⁰ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical.

Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The term “heteroalkyl”, as used herein, refers to a saturated or unsaturated chain of carbon atoms and at least one heteroatom, wherein no two heteroatoms are adjacent.

The terms “heteroaryl” and “hetaryl” include substituted or unsubstituted aromatic single ring structures, preferably 3- to 10-membered rings, more preferably 5- to 9-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

Individual rings of such multicyclic heteroaryl groups can have different connectivities, e.g., fused, etc. in addition to covalent bond substitution. Exemplary heteroaryl groups include furyl, thienyl, thiazolyl, pyrazolyl, isothiazolyl, oxazolyl, isoxazolyl, pyrrolyl, triazolyl, tetrazolyl, imidazolyl, 1,3,5-oxadiazolyl, 1,2,4-oxadiazolyl, 1,2,3-oxadiazolyl, 1,3,5-thiadiazolyl, 1,2,3-thiadiazolyl, 1,2,4-thiadiazolyl, pyridyl, pyrimidyl, pyrazinyl, pyridazinyl, 1,2,4-triazinyl, 1,2,3-triazinyl, 1,3,5-triazinyl, pyrazolo[3,4-b]pyridinyl, cinnolinyl, pteridinyl, purinyl, 6,7-dihydro-5H-[1]pyrindinyl, benzo[b]thiophenyl, 5,6,7,8-tetrahydro-quinolin-3-yl, benzoxazolyl, benzothiazolyl, benzisothiazolyl, benzisoxazolyl, benzimidazolyl, thianaphthenyl, isothianaphthenyl, benzofuranyl, isobenzofuranyl, isoindolyl, indolyl, indolizinyl, indazolyl, isoquinolyl, quinolyl, phthalazinyl, quinoxalinyl, quinazolinyl and benzoxazinyl, etc. In general, the heteroaryl group typically is attached to the main structure via a carbon atom.

The term “heteroatom” as used herein means an atom of any element other than carbon or hydrogen. Preferred heteroatoms are nitrogen, oxygen, and sulfur.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

Individual rings of such multicyclic heterocycloalkyl groups can have different connectivities, e.g., fused, bridged, spiro, etc. in addition to covalent bond substitution.

Exemplary heterocycloalkyl groups include pyrrolidinyl, tetrahydrofuranyl, dihydrofuranyl, tetrahydropyranyl, pyranyl, thiopyranyl, azindinyl, azetidinyl, oxiranyl, methylenedioxyl, chromenyl, barbituryl, isoxazolidinyl, 1,3-oxazolidin-3-yl, isothiazolidinyl, 1,3-thiazolidin-3-yl, 1,2-pyrazolidin-2-yl, 1,3-pyrazolidin-1-yl, piperidinyl, thiomorpholinyl, 1,2-tetrahydrothiazin-2-yl, 1,3-tetrahydrothiazin-3-yl, tetrahydrothiadiazinyl, morpholinyl, 1,2-tetrahydrodiazin-2-yl, 1,3-tetrahydrodiazin-1-yl, tetrahydroazepinyl, piperazinyl, piperizin-2-onyl, piperizin-3-onyl, chromanyl, 2-pyrrolinyl, 3-pyrrolinyl, imidazolidinyl, 2-imidazolidinyl, 1,4-dioxanyl, 8-azabicyclo[3.2.1]octanyl, 3-azabicyclo[3.2.1]octanyl, 3,8-diazabicyclo[3.2.1]octanyl, 2,5-diazabicyclo[2.2.1]heptanyl, 2,5-diazabicyclo[2.2.2]octanyl, octahydro-2H-pyrido[1,2-a]pyrazinyl, 3-azabicyclo[4.1.0]heptanyl, 3-azabicyclo[3.1.0]hexanyl 2-azaspiro[4.4]nonanyl, 7-oxa-1-aza-spiro[4.4]nonanyl, 7-azabicyclo[2.2.2]heptanyl, octahydro-1H-indolyl, etc. In general, the heterocycloalkyl group typically is attached to the main structure via a carbon atom or a nitrogen atom.

The term “heterocyclylalkyl”, as used herein, refers to an alkyl group substituted with a heterocycle group.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms.

Thus, groups like methyl, ethoxyethyl, 2-pyridyl, and trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocyclyl, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer non-hydrogen atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “silyl” refers to a silicon moiety with three hydrocarbyl moieties attached thereto.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxy, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that substituents can themselves be substituted, if appropriate. Unless specifically stated as “unsubstituted,” references to chemical moieties herein are understood to include substituted variants. For example, reference to an “aryl” group or moiety implicitly includes both substituted and unsubstituted variants.

The term “sulfate” is art-recognized and refers to the group —OSO₃H, or a pharmaceutically acceptable salt thereof.

The term “sulfonamide” is art-recognized and refers to the group represented by the general formulae

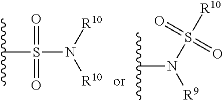

wherein R⁹ and R¹⁰ independently represents hydrogen or hydrocarbyl, such as alkyl, or R⁹ and R¹⁰ taken together with the intervening atom(s) complete a heterocycle having from 4 to 8 atoms in the ring structure.

The term “sulfoxide” is art-recognized and refers to the group —S(O)—R¹⁰, wherein R¹⁰ represents a hydrocarbyl.

The term “sulfonate” is art-recognized and refers to the group SO₃H, or a pharmaceutically acceptable salt thereof.

The term “sulfone” is art-recognized and refers to the group —S(O)₂—R¹⁰, wherein R¹⁰ represents a hydrocarbyl.

The term “thioalkyl”, as used herein, refers to an alkyl group substituted with a thiol group.

The term “thioester”, as used herein, refers to a group —C(O)SR¹⁰ or —SC(O)R¹⁰ wherein R¹⁰ represents a hydrocarbyl.

The term “thioether”, as used herein, is equivalent to an ether, wherein the oxygen is replaced with a sulfur.

The term “urea” is art-recognized and may be represented by the general formula

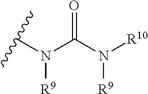

wherein R⁹ and R¹⁰ independently represent hydrogen or a hydrocarbyl, such as alkyl, or either occurrence of R⁹ taken together with R¹⁰ and the intervening atom(s) complete a heterocycle having from 4 to 8 atoms in the ring structure.

The term “protecting group” refers to a group of atoms that, when attached to a reactive functional group in a molecule, mask, reduce or prevent the reactivity of the functional group. Typically, a protecting group may be selectively removed as desired during the course of a synthesis. Examples of protecting groups can be found in Greene and Wuts, Protective Groups in Organic Chemistry, 3^{rd} Ed., 1999, John Wiley & Sons, NY and Harrison et al., Compendium of Synthetic Organic Methods, Vols. 1-8, 1971-1996, John Wiley & Sons, NY. Representative nitrogen protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“TES”), trityl and substituted trityl groups, allyloxycarbonyl, 9-fluorenylmethyloxycarbonyl (“FMOC”), nitro-veratryloxycarbonyl (“NVOC”) and the like. Representative hydroxyl protecting groups include, but are not limited to, those where the hydroxyl group is either acylated (esterified) or alkylated such as benzyl and trityl ethers, as well as alkyl ethers, tetrahydropyranyl ethers, trialkylsilyl ethers (e.g., TMS or TIPS groups), glycol ethers, such as ethylene glycol and propylene glycol derivatives and allyl ethers.

The invention also includes various isomers and mixtures thereof. Certain of the compounds of the present invention may exist in various stereoisomeric forms. Stereoisomers are compounds which differ only in their spatial arrangement. Enantiomers are pairs of stereoisomers whose mirror images are not superimposable, most commonly because they contain an asymmetrically substituted carbon atom that acts as a chiral center. “Enantiomer” means one of a pair of molecules that are mirror images of each other and are not superimposable. Diastereomers are stereoisomers that are not related as mirror images, most commonly because they contain two or more asymmetrically substituted carbon atoms. “R” and “S” represent the configuration of substituents around one or more chiral carbon atoms. When a chiral center is not defined as R or S, either a pure enantiomer or a mixture of both configurations is present.

“Racemate” or “racemic mixture” means a compound of equimolar quantities of two enantiomers, wherein such mixtures exhibit no optical activity; i.e., they do not rotate the plane of polarized light. In certain embodiments, compounds of the invention may be racemic.

In certain embodiments, compounds of the invention may be enriched in one enantiomer. For example, a compound of the invention may have greater than about 30% ee, about 40% ee, about 50% ee, about 60% ee, about 70% ee, about 80% ee, about 90% ee, or even about 95% or greater ee. In certain embodiments, compounds of the invention may have more than one stereocenter. In certain such embodiments, compounds of the invention may be enriched in one or more diastereomer. For example, a compound of the invention may have greater than about 30% de, about 40% de, about 50% de, about 60% de, about 70% de, about 80% de, about 90% de, or even about 95% or greater de.

In certain embodiments, the therapeutic preparation may be enriched to provide predominantly one enantiomer of a compound (e.g., of Formula I). An enantiomerically enriched mixture may comprise, for example, at least about 60 mol percent of one enantiomer, or more preferably at least about 75, about 90, about 95, or even about 99 mol percent. In certain embodiments, the compound enriched in one enantiomer is substantially free of the other enantiomer, wherein substantially free means that the substance in question makes up less than about 10%, or less than about 5%, or less than about 4%, or less than about 3%, or less than about 2%, or less than about 1% as compared to the amount of the other enantiomer, e.g., in the composition or compound mixture. For example, if a composition or compound mixture contains about 98 grams of a first enantiomer and about 2 grams of a second enantiomer, it would be said to contain about 98 mol percent of the first enantiomer and only about 2% of the second enantiomer.

In certain embodiments, the therapeutic preparation may be enriched to provide predominantly one diastereomer of a compound (e.g., of Formula I). A diastereomerically enriched mixture may comprise, for example, at least about 60 mol percent of one diastereomer, or more preferably at least about 75, about 90, about 95, or even about 99 mol percent.

The compounds of the invention may be prepared as individual isomers by either isomer specific synthesis or resolved from an isomeric mixture. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods. When the stereochemistry of a disclosed compound is named or depicted by structure, the named or depicted stereoisomer is at least about 60%, about 70%, about 80%, about 90%, about 99% or about 99.9% by weight pure relative to the other stereoisomers. When a single enantiomer is named or depicted by structure, the depicted or named enantiomer is at least about 60%, about 70%, about 80%, about 90%, about 99% or about 99.9% by weight optically pure. Percent optical purity by weight is the ratio of the weight of the enantiomer that is present divided by the combined weight of the enantiomer that is present and the weight of its optical isomer.

In the pictorial representation of the compounds given through this application, a thickened tapered line ( [symbol] ) indicates a substituent which is above the plane of the ring to which the asymmetric carbon belongs and a dotted line ( [symbol] ) indicates a substituent which is below the plane of the ring to which the asymmetric carbon belongs.

As used herein a compound of the present invention can be in the form of one of the possible isomers, rotamers, atropisomers, tautomers or mixtures thereof, for example, as substantially pure geometric (cis or trans) isomers, diastereomers, optical isomers (antipodes), racemates or mixtures thereof.

An isotope-labelled form of a disclosed compound has one or more atoms of the compound replaced by an atom or atoms having an atomic mass or mass number different that that which usually occurs in greater natural abundance. Examples of isotopes which are readily commercially available and which can be incorporated into a disclosed compound by well-known methods include isotopes of hydrogen, carbon, nitrogen, oxygen, phosphorus, fluorine and chlorine, for example, 2H, 3H, 13C, 14C, 15N, 18O, 17O, 31P, 32P, 35S, 18F and 36Cl, respectively. An isotope-labelled compound provided herein can usually be prepared by carrying out the procedures disclosed herein, replacing a non-isotope-labelled reactant by an isotope-labelled reactant.

The concentration of such a heavier isotope, specifically deuterium, may be defined by the isotopic enrichment factor. The term “isotopic enrichment factor” as used herein means the ratio between the isotopic abundance and the natural abundance of a specified isotope. If a hydrogen atom in a compound of this invention is replaced with deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

An isotope-labelled compound as provided herein can be used in a number of beneficial ways. Compounds having 14C incorporated are suitable for medicament and/or substrate tissue distribution assays. Tritium (3H) and carbon-14 (14C), are preferred isotopes owing to simple preparation and excellent detectability. Heavier isotopes, for example deuterium (2H), has therapeutic advantages owing to the higher metabolic stability. Metabolism is affected by the primary kinetic isotope effect, in which the heavier isotope has a lower ground state energy and causes a reduction in the rate-limiting bond breakage. Slowing the metabolism can lead to an increased in vivo half-life or reduced dosage requirements or an improvement in therapeutic index.

For a further discussion, see S. L. Harbeson and R. D. Tung, Deuterium In Drug Discovery and Development, Ann. Rep. Med. Chem. 2011, 46, 403-417, Foster, A. B., “Deuterium Isotope Effects in Studies of Drug Metabolism,” Trends in Pharmacological Sciences, 5: 524-527 (1984) AND Foster, A. B., “Deuterium Isotope Effects in the Metabolism of Drugs and Xenobiotics: Implications for Drug Design,” Advances in Drug Research, 14: 1-40 (1985).

Metabolic stability can be affected by the compound's processing in different organs of the body. For example, compounds with poor pharmacokinetic profiles are susceptible to oxidative metabolism. In vitro liver microsomal assays currently available provide valuable information on the course of oxidative metabolism of this type, which in turn assists in the rational design of deuterated compounds as disclosed herein. Improvements can be measured in a number of assays known in the art, such as increases in the in vivo half-life (t1/2), concentration at maximum therapeutic effect (Cmax), area under the dose response curve (AUC), and bioavailability; and in terms of reduced clearance, dose and materials costs. Another effect of deuterated compounds can be diminishing or eliminating undesired toxic metabolites. For example, if a toxic metabolite arises through oxidative carbon-hydrogen (C—H) bond cleavage, the deuterated analogue will have a slower reaction time and slow the production of the unwanted metabolite, even if the particular oxidation is not a rate-determining step. See, e.g., Hanzlik et al., J. Org. Chem. 55, 3992-3997, 1990, Reider et al., J. Org. Chem. 52, 3326-3334, 1987, Foster, Adv. Drug Res. 14, 1-40, 1985, Gillette et al, Biochemistry 33(10) 2927-2937, 1994, and Jarman et al. Carcinogenesis 16(4), 683-688, 1993.

The term “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or other primates (e.g., cynomolgus monkeys, rhesus monkeys); mammals, including commercially relevant mammals such as cattle, pigs, horses, sheep, goats, cats, and/or dogs; and/or birds, including commercially relevant birds such as chickens, ducks, geese, quail, and/or turkeys. Preferred subjects are humans.

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

The term “treating” means to decrease, suppress, attenuate, diminish, arrest, or stabilize the development or progression of a disease (e.g., a disease or disorder delineated herein), lessen the severity of the disease or improve the symptoms associated with the disease. Treatment includes treating a symptom of a disease, disorder or condition. Without being bound by any theory, in some embodiments, treating includes augmenting deficient CFTR activity. If it is administered prior to clinical manifestation of the unwanted condition (e.g., disease or other unwanted state of the subject) then the treatment is prophylactic (i.e., it protects the subject against developing the unwanted condition), whereas if it is administered after manifestation of the unwanted condition, the treatment is therapeutic, (i.e., it is intended to diminish, ameliorate, or stabilize the existing unwanted condition or side effects thereof).

As used herein, the term “prodrug” means a pharmacological derivative of a parent drug molecule that requires biotransformation, either spontaneous or enzymatic, within the organism to release the active drug. For example, prodrugs are variations or derivatives of the compounds of the invention that have groups cleavable under certain metabolic conditions, which when cleaved, become the compounds of the invention. Such prodrugs then are pharmaceutically active in vivo, when they undergo solvolysis under physiological conditions or undergo enzymatic degradation. Prodrug compounds herein may be called single, double, triple, etc., depending on the number of biotransformation steps required to release the active drug within the organism, and the number of functionalities present in a precursor-type form.

Prodrug forms often offer advantages of solubility, tissue compatibility, or delayed release in the mammalian organism (See, Bundgard, Design of Prodrugs, pp. 7-9, 21-24, Elsevier, Amsterdam 1985 and Silverman, The Organic Chemistry of Drug Design and Drug Action, pp. 352-401, Academic Press, San Diego, CA, 1992). Prodrugs commonly known in the art include well-known acid derivatives, such as, for example, esters prepared by reaction of the parent acids with a suitable alcohol, amides prepared by reaction of the parent acid compound with an amine, basic groups reacted to form an acylated base derivative, etc. Of course, other prodrug derivatives may be combined with other features disclosed herein to enhance bioavailability.

As such, those of skill in the art will appreciate that certain of the presently disclosed compounds having free amino, amido, hydroxy or carboxylic groups can be converted into prodrugs. Prodrugs include compounds having an amino acid residue, or a polypeptide chain of two or more (e.g., two, three or four) amino acid residues which are covalently joined through peptide bonds to free amino, hydroxy or carboxylic acid groups of the presently disclosed compounds. The amino acid residues include the 20 naturally occurring amino acids commonly designated by three letter symbols and also include 4-hydroxyproline, hydroxylysine, demosine, isodemosine, 3-methylhistidine, norvalin, beta-alanine, gamma-aminobutyric acid, citrullinehomocysteine, homoserine, ornithine and methionine sulfone. Prodrugs also include compounds having a carbonate, carbamate, amide or alkyl ester moiety covalently bonded to any of the above substituents disclosed herein.

A “therapeutically effective amount”, as used herein refers to an amount that is sufficient to achieve a desired therapeutic effect. For example, a therapeutically effective amount can refer to an amount that is sufficient to improve at least one sign or symptom of cystic fibrosis.

A “response” to a method of treatment can include a decrease in or amelioration of negative symptoms, a decrease in the progression of a disease or symptoms thereof, an increase in beneficial symptoms or clinical outcomes, a lessening of side effects, stabilization of disease, partial or complete remedy of disease, among others.

As used herein, “CFTR” means cystic fibrosis transmembrane conductance regulator. Loss of function mutations of CFTR are a cause of cystic fibrosis and lead to exocrine gland dysfunction and abnormal mucocilliary clearance. Mutations in the CFTR gene or protein may result in reduced activity of CFTR. The most common mutation is a specific mutation of the deletion of three nucleotides of the codon for phenylalanine at positon 508 (about 70% of cystic fibrosis patients) referred to as “ΔF508”. The ΔF508 mutation decreases the stability of the CFTR NBD1 domain and limits CFTR interdomain assembly. A patient can be ΔF508 homozygous or ΔF508 heterozygous (ΔF508/ΔF508). In particular mutations, the result is a gating mutation leading to a low probability of the ion channel in the open position. Such mutations include but are not limited to G551D, G178R, S549N, S549R, G551S, G970R, G1244E, 51251N, 51255P, and G1349D.

As used herein, the term “CFTR modulator” refers to a compound that increases the activity of CFTR. In certain aspects, a CFTR modulator is a CFTR corrector or a CFTR potentiator or a dual-acting compound having activities of a corrector and a potentiator. These dual acting agents are useful when the mutations result in absence or reduced amount of synthesized CFTR protein.

As used herein, the term “CFTR corrector” refers to a compound that increases the amount of functional CFTR protein to the cell surface and thus enhances ion transport. The CFTR correctors partially “rescue” misfolding of CFTR, thereby permitting its maturation and functional expression on the cell surface and may modify the folding environment and compounds that interact directly to modify folding and conformational maturation during synthesis. Examples of correctors include, but are not limited to, VX-809, VX-661, VX-152, VX-440, VX-983, and GLPG2222.

As used herein, the term “CFTR potentiator” refers to a compound that increases the ion channel activity of CFTR protein located at the cell surface, resulting in enhanced ion transport. CFTR potentiators repair the defective channel functions caused by mutations. Examples of potentiators include, but are not limited to, ivacaftor (VX770), deuterated ivacaftor (CPT 656), genestien and GLPG1837.

As used herein, the term “CTFR pharmacological chaperone” (PC) refers to compounds that stabilize the CTFR protein in its native state by binding directly to the protein.

As used herein, the term “CTFR proteostasis regulator” (PR) refers to compounds that enhance the protein folding efficiency within the cell. PRs can alter the activity of transcriptional, folding and/or membrane trafficking machinery, as well as impeding the degradation of partially folded, but functional, conformers at the endoplasmic reticulum (ER) or plasma membrane.

As used herein, “CFTR disease or condition” refers to a disease or condition associated with deficient CFTR activity, for example, cystic fibrosis, congenital bilateral absence of vas deferens (CBAVD), acute, recurrent, or chronic pancreatitis, disseminated bronchiectasis, asthma, allergic pulmonary aspergillosis, smoking-related lung diseases, such as chronic obstructive pulmonary disease (COPD), chronic sinusitis, dry eye disease, protein C deficiency, A.beta.-lipoproteinemia, lysosomal storage disease, type 1 chylomicronemia, mild pulmonary disease, lipid processing deficiencies, type 1 hereditary angioedema, coagulation-fibrinolyis, hereditary hemochromatosis, CFTR-related metabolic syndrome, chronic bronchitis, constipation, pancreatic insufficiency, hereditary emphysema, and Sjogren's syndrome.

### Methods of Use

As discussed above, CTFR is composed of two six membrane-spanning domains (MSD1 and MSD2), two nucleotide bind domains (NBD1 and NBD2), a regulatory region (R) and four cytosolic loops (CL1-4). CFTR protein is located primarily in the apical membrane of epithelial cells where it functions to conduct anions, including chloride, bicarbonate and thiocyanate into and out of the cell. The most frequent CFTR mutation is the in-frame deletion of phenylalanine at residue 508 (ΔF508) in the first nucleotide binding domain (NBD1). The mutation has several deleterious effects on the production of CFTR in the ER, its correct folding, its movement to the plasma membrane and its normal function as an ion channel for the cell.

One such negative effect is that the NBD1 domain is partially or mis-folded which is recognized within the cell as an aberrant protein and tagged for disposal by ER-associated degradation (ERAD) via the ubiquitin-proteasome system (UPS). Should a partially or mis-folded CFTR protein emerge from the ER, the protein must travel to the plasma membrane through complex glycosylation in the Golgi compartment and be functionally inserted. In wild-type CFTR, only 20-40% of CTFR reaches the plasma membrane, indicating that CTFR has energetic instability of individual NBDs, a slow domain assembly, and relatively fast ERAD kinetics which all contribute to inefficient folding and sensitize CFTR to structural perturbations by mutations.

In wild-type CTFR, the NBD1 domain folds co-translationally while other domains fold post-translationally. Mutated ΔF508 CTFR has impaired NBD1 folding but its backbone structure and thermodynamic stability are similar to wild-type CTFR. With delayed folding kinetics, mutated ΔF508 CTFR NBD1 has an increased folding activation energy. Lack of proper folding results in hydrophobic residues being exposed to the surface of NBD1 which causes aggregation with other CTFR proteins. Thus, the aggregation temperature of mutated CTFR drops from 41° C. to 33° C. This level of instability creates a greater percentage of mis-folded mutant CFTR at physiological temperature (37° C. in humans). Mutant CFTR suffers from both kinetic and thermodynamic folding defects. CFTR correctors and regulators can address these folding defects, but complete energetic correction of mutant NBD1 folding has been shown to not result in the CTFR biosynthetic processing, underscoring the need for interface stability as well.

The disclosed CFTR correctors can interact with the NBD domain to stabilize the correct folded position, such that CTFR is not labeled for elimination from the cell. The preservation of correct folding enables CTFR to function as a chloride ion channel at wild-type levels. In some embodiments, disclosed CFTR correctors can enhance the performance of wild-type CTFR.

Disclosed herein are CFTR correctors that promote A508 CFTR exit from the ER and accumulation in the plasma membrane. Increasing the amount of CFTR cell surface expression can result in improved chloride conductance following channel activation by both potentiators and a cAMP agonist. Thus, disclosed herein are combinations of CFTR correctors and potentiators, optionally with cAMP agonists or another therapeutic agent as described below.

In some instances, the correctors disclosed herein are dual correctors, which exhibit CFTR-stabilizing activity in NBD1 domain, as well as at the interface of the NBD1 domain and ICL4 domain.

Disclosed herein are methods of treating deficient CFTR activity in a cell, comprising contacting the cell with a compound described herein, e.g. a compound of any one of formula I, IA, IB, IC, ID, IE or II, or a pharmaceutically acceptable salt thereof. In certain embodiments, contacting the cell occurs in a subject in need thereof, thereby treating a disease or disorder mediated by deficient CFTR activity.

Also, disclosed herein are methods of treating a disease or a disorder mediated by deficient CFTR activity comprising administering a compound of Formula I or a pharmaceutically acceptable salt thereof. In some embodiments, the subject is a mammal, preferably a human. In some embodiments, the disease is associated with the regulation of fluid volumes across epithelial membranes, particularly an obstructive airway disease such as CF or COPD.

Such diseases and conditions include, but are not limited to, cystic fibrosis, asthma, smoke induced COPD, chronic bronchitis, rhinosinusitis, constipation, pancreatitis, pancreatic insufficiency, male infertility caused by congenital bilateral absence of the vas deferens (CBAVD), mild pulmonary disease, idiopathic pancreatitis, allergic bronchopulmonary aspergillosis (ABPA), liver disease, hereditary emphysema, hereditary hemochromatosis, coagulation-fibrinolysis deficiencies, protein C deficiency, Type 1 hereditary angioedema, lipid processing deficiencies, familial hypercholesterolemia, Type 1 chylomicronemia, abetalipoproteinemia, lysosomal storage diseases, I-cell disease/pseudo-Hurler, mucopolysaccharidoses, Sandhof/Tay-Sachs, Crigler-Najjar type II, polyendocrinopathy/hyperinsulemia, Diabetes mellitus, Laron dwarfism, myleoperoxidase deficiency, primary hypoparathyroidism, melanoma, glycanosis CDG type 1, congenital hyperthyroidism, osteogenesis imperfecta, hereditary hypofibrinogenemia, ACT deficiency, Diabetes insipidus (DI), neurophyseal DI, neprogenic DI, Charcot-Marie Tooth syndrome, Perlizaeus-Merzbacher disease, neurodegenerative diseases, Alzheimer's disease, Parkinson's disease, amyotrophic lateral sclerosis, progressive supranuclear plasy, Pick's disease, several polyglutamine neurological disorders, Huntington's, spinocerebullar ataxia type I, spinal and bulbar muscular atrophy, dentatorubal pallidoluysian, myotonic dystrophy, spongiform encephalopathies, hereditary Creutzfeldt-Jakob disease, Fabry disease, Straussler-Scheinker syndrome, COPD, dry-eye disease, Sjogren's disease, Osteoporosis, Osteopenia, bone healing and bone growth, bone repair, bone regeneration, reducing bone resorption, increasing bone deposition, Gorham's Syndrome, chloride channelopathies, myotonia congenita, Bartter's syndrome type III, Dent's disease, hyperekplexia, epilepsy, hyperekplexia, lysosomal storage disease, Angelman syndrome, Primary Ciliary Dyskinesia (PCD), PCD with situs inversus, PCD without situs inversus and ciliary aplasia.

Such diseases and conditions include, but are not limited to, cystic fibrosis, congenital bilateral absence of vas deferens (CBAVD), acute, recurrent, or chronic pancreatitis, disseminated bronchiectasis, asthma, allergic pulmonary aspergillosis, chronic obstructive pulmonary disease (COPD), chronic sinusitis, dry eye disease, protein C deficiency, Abetalipoproteinemia, lysosomal storage disease, type 1 chylomicronemia, mild pulmonary disease, lipid processing deficiencies, type 1 hereditary angioedema, coagulation-fibrinolyis, hereditary hemochromatosis, CFTR-related metabolic syndrome, chronic bronchitis, constipation, pancreatic insufficiency, hereditary emphysema, and Sjogren's syndrome. In some embodiments, the disease is cystic fibrosis.

Provided herein are methods of treating cystic fibrosis, comprising administering to a subject in need thereof, a compound as disclosed herein or a pharmaceutically acceptable salt thereof. Also provided herein are methods of lessening the severity of cystic fibrosis, comprising administering to a subject in need thereof, a compound as disclosed herein or a pharmaceutically acceptable salt thereof. In some embodiments, the subject is a human. In some embodiments, the subject is at risk of developing cystic fibrosis, and administration is carried out prior to the onset of symptoms of cystic fibrosis in the subject.

Provided herein are compounds as disclosed herein for use in treating a disease or condition mediated by deficient CFTR activity. Also provided herein are uses of a compound as disclosed herein for the manufacture of a medicament for treating a disease or condition mediated by deficient CFTR activity.

Provided herein are kits for use in measuring the activity of CFTR or a fragment thereof in a biological sample in vitro or in vivo. The kit can contain: (i) a compound as disclosed herein, or a pharmaceutical composition comprising the disclosed compound, and (ii) instructions for: a) contacting the compound or composition with the biological sample; and b) measuring activity of said CFTR or a fragment thereof. In some embodiments, the biological sample is biopsied material obtained from a mammal or extracts thereof; blood, saliva, urine, feces, semen, tears, other body fluids, or extracts thereof. In some embodiments, the mammal is a human.

### Combination Treatments

As used herein, the term “combination therapy” means administering to a subject (e.g., human) two or more CFTR modulators, or a CFTR modulator and an agent such as antibiotics, ENaC inhibitors, GSNO (S-nitrosothiol s-nitroglutanthione) reductase inhibitors, and a CRISPR Cas correction therapy or system (as described in US 2007/0022507 and the like).

In certain embodiments, the method of treating or preventing a disease or condition mediated by deficient CFTR activity comprises administering a compound as disclosed herein conjointly with one or more other therapeutic agent(s). In some embodiments, one other therapeutic agent is administered. In other embodiments, at least two other therapeutic agents are administered.

Additional therapeutic agents include, for example, ENaC inhibitors, mucolytic agents, modulators of mucus rheology, bronchodilators, antibiotics, anti-infective agents, anti-inflammatory agents, ion channel modulating agents, therapeutic agents used in gene or mRNA therapy, agents that reduce airway surface liquid and/or reduce airway surface PH, CFTR correctors, and CFTR potentiators, or other agents that modulate CFTR activity. Other therapeutics include liposomal composition compnents such as those described in WO2012/170889, hybrid oligonucleotides that facilitate RNA cleavage such as those described in WO2016/130943, and single stranded oligonucleotides that modulate gene expression as described in WO2016/130929.

In some embodiments, at least one additional therapeutic agent is selected from one or more CFTR modulators, one or more CFTR correctors and one or more CFTR potentiators.

Non-limiting examples of additional therapeutics include VX-770 (Ivacaftor), VX-809 (Lumacaftor, 3-(6-(I-(2,2-5 difluorobenzo[d][1,3]dioxo1-5-yl)cyclopropanecarboxamido)-3-methylpyridin-2-yl) benzoic acid, VX-661 (Tezacaftor, I-(2,2-difluoro-1,3-benzodioxol-5-yl)-N—[I-[(2R)-2,3-dihydroxypropyl]-6-fluoro-2-(2-hydroxy-1, I-dimethylethyl)-IH-indol-5-yl]-cyclopropanecarboxamide), VX-983, VX-152, VX-440, VX-445, VX-659, VX-371, Orkambi, Ataluren (PTC 124) (3-[5-(2-fluorophenyl)-1,2,4-oxadiazo1-3-yl]benzoic acid), PTI-130 (Proteostasis), PTI-801, PTI-808, PTI-428, N91115.74 (cavosonstat), QBW251 (Novartis) compounds described in WO2011113894, compounds N30 Pharmaceuticals (e.g., WO 2014/186704), deuterated ivacaftor (e.g., CTP-656 or VX-561), GLPG 2222, GLPG2451, GLPG3067, GLPG2851, GLPG2737, GLPG 1837 (N-(3-carbamoyl-5,5,7,7-tetramethyl-5,7-dihydro-4H-thieno[2,3-c]pyran-2-yl)-1H-pyrazole-5-carboxamide), GLPG 2665 (Galapagos), FDL 169 (Flatley Discovery lab), FDL 176, FDL438, FDL304, FD2052160, FD1881042, FD2027304, FD2035659, FD2033129, FD1860293, CFFT-Pot01, CFFT-Pot-02, P-1037, glycerol, phenylbutyrate, and the like.

Non-limiting examples of additional therapeutics include compounds disclosed in US Patent Application Nos. 62/944,141, 62/944,158 and 62/944,188, each of which is incorporated by reference herein in its entirety.

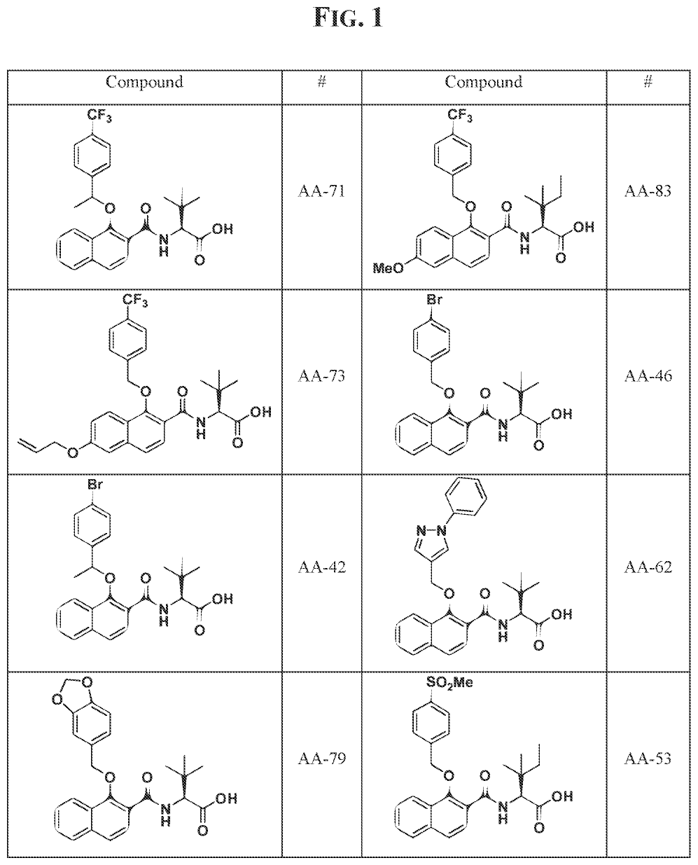

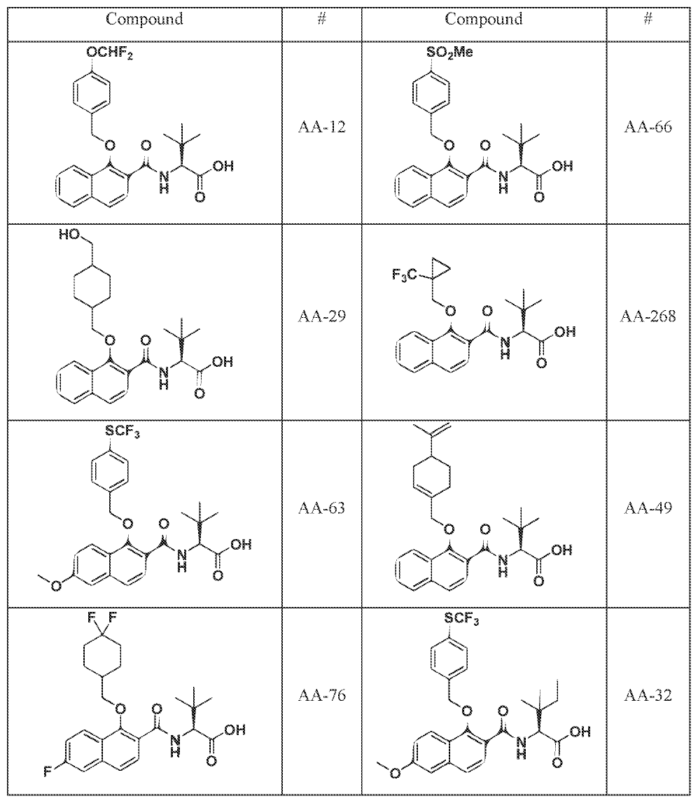

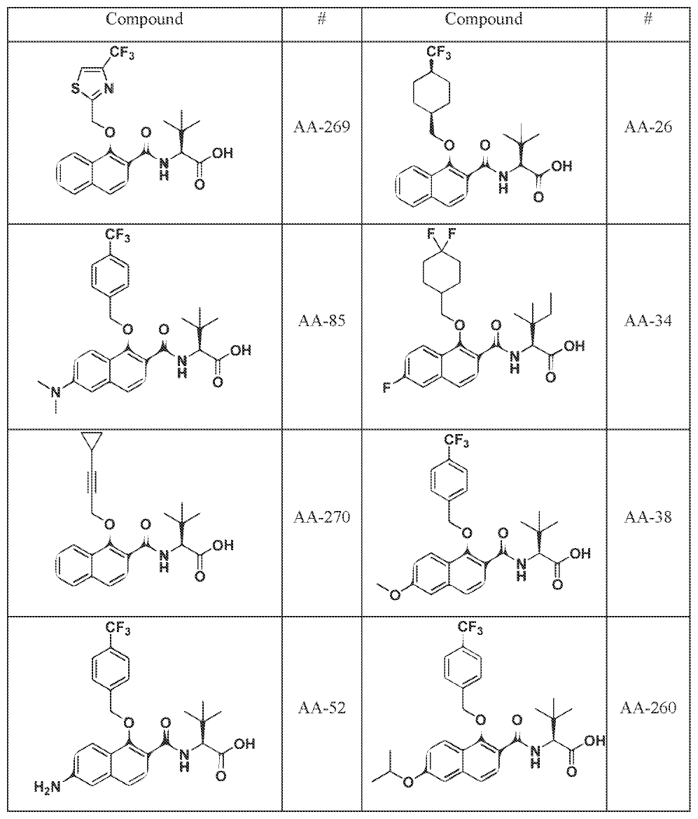

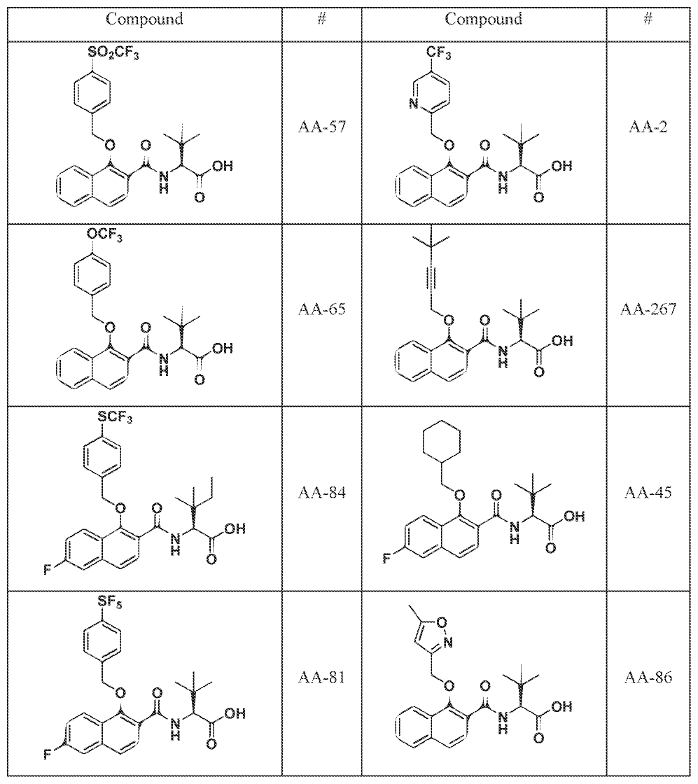

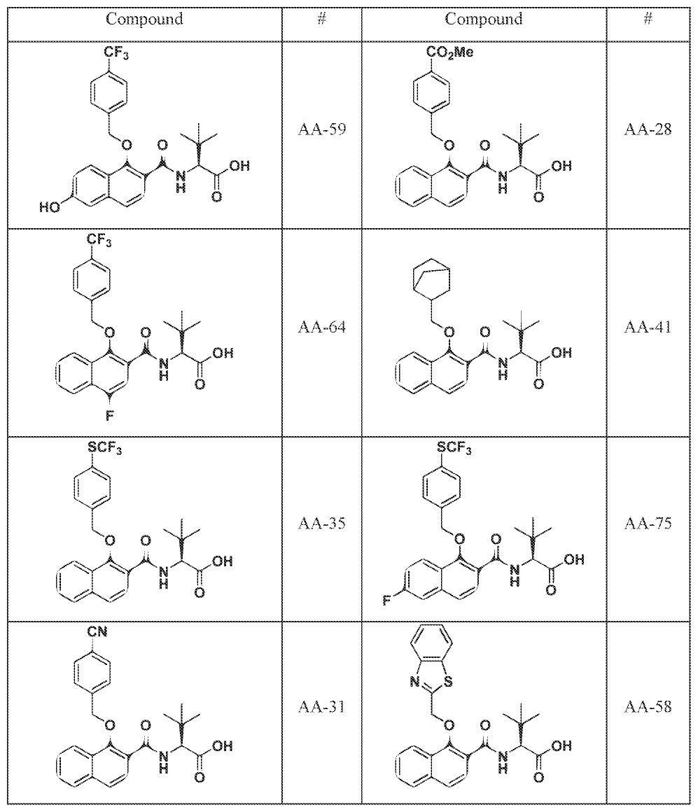

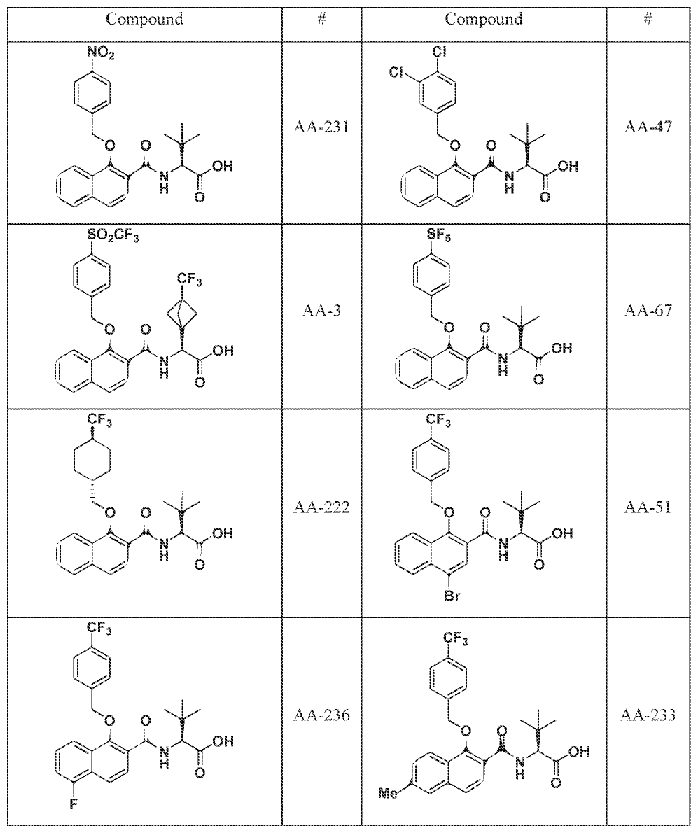

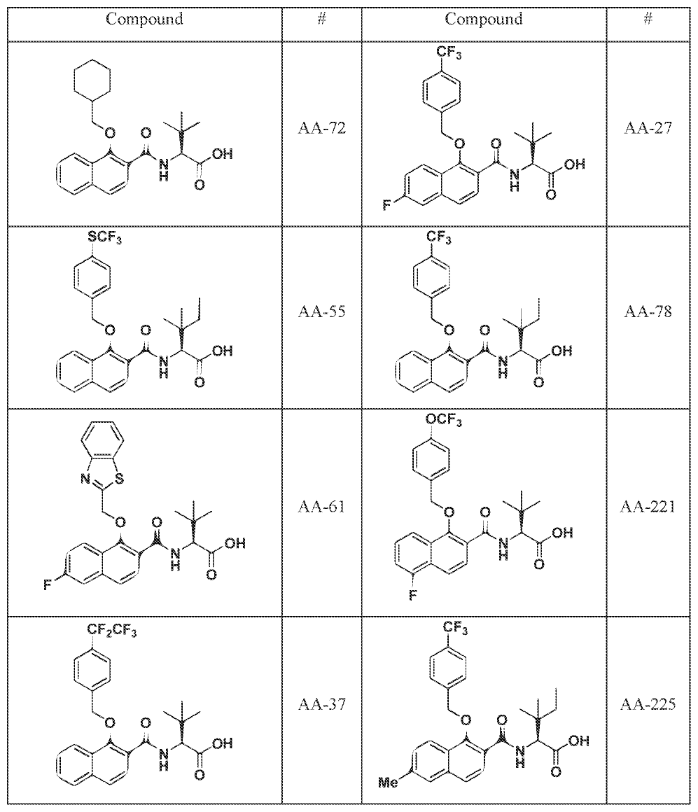

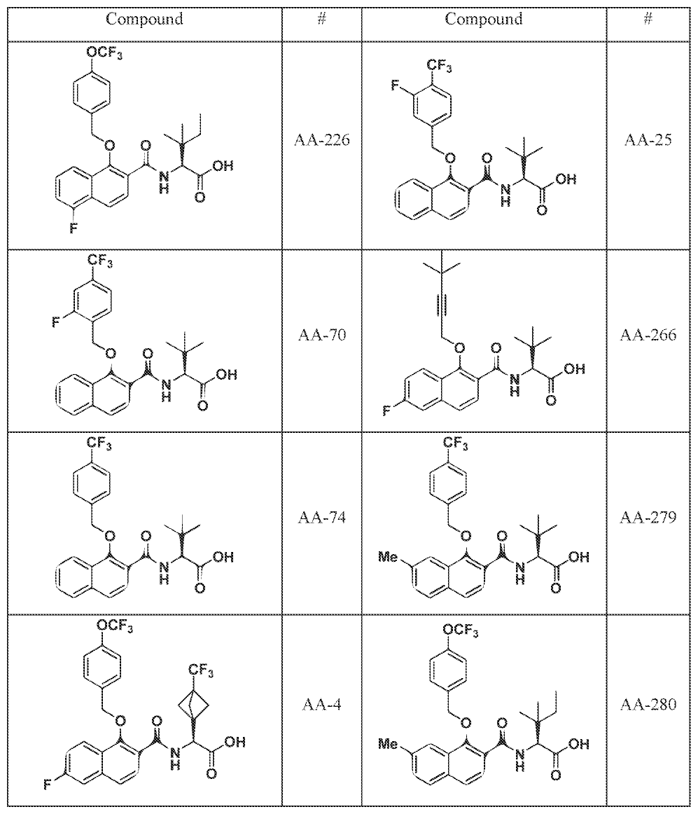

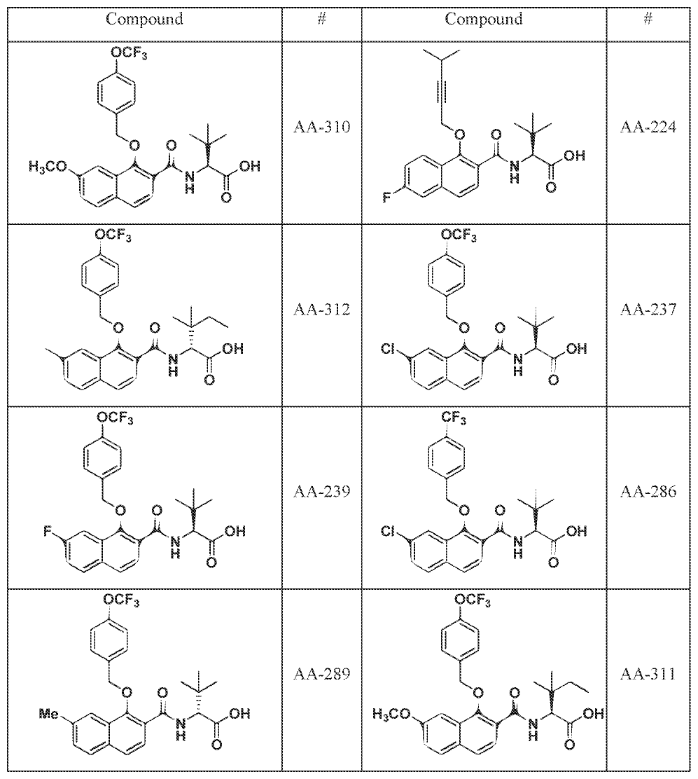

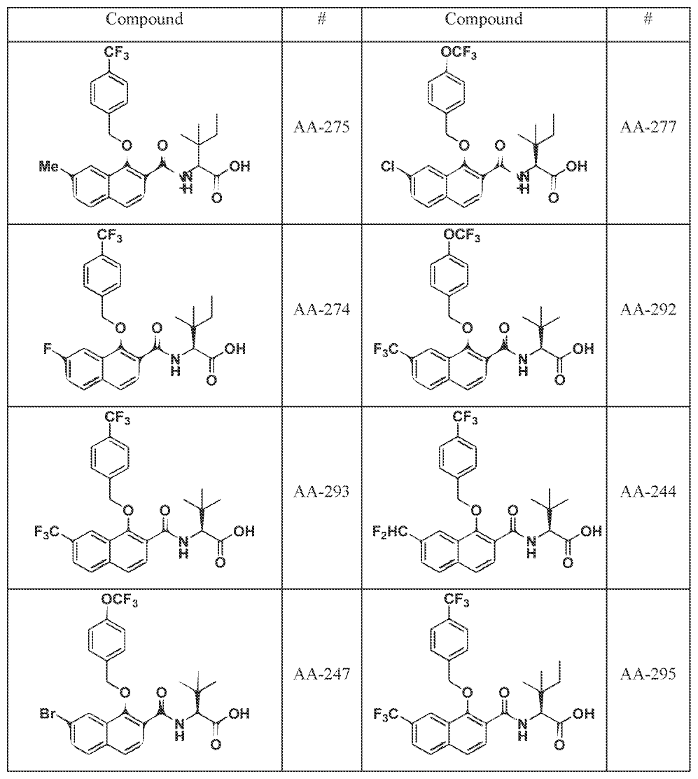

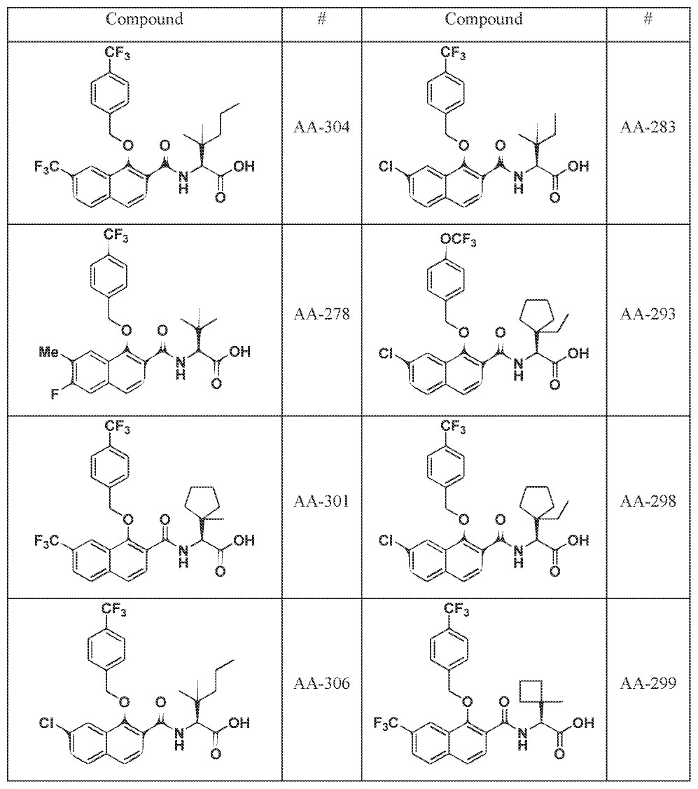

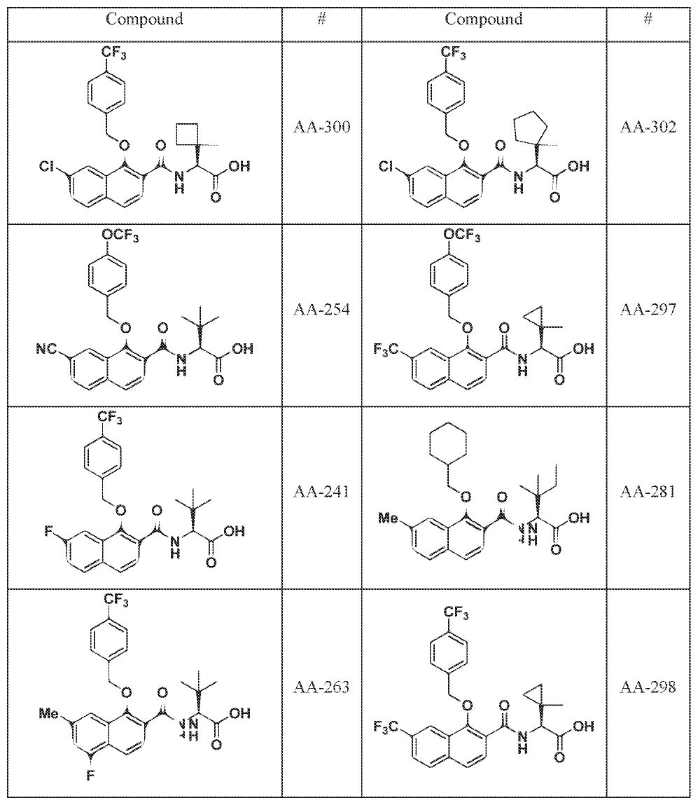

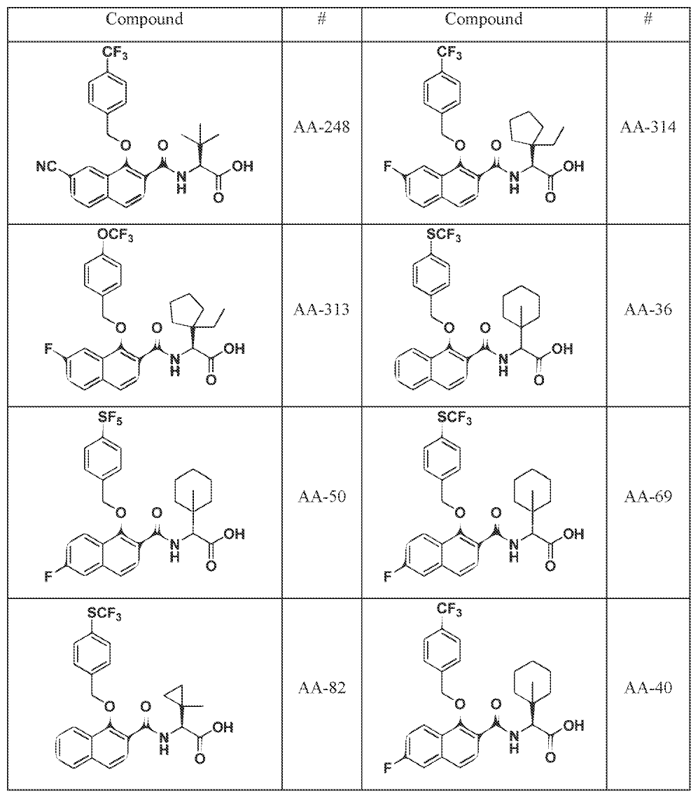

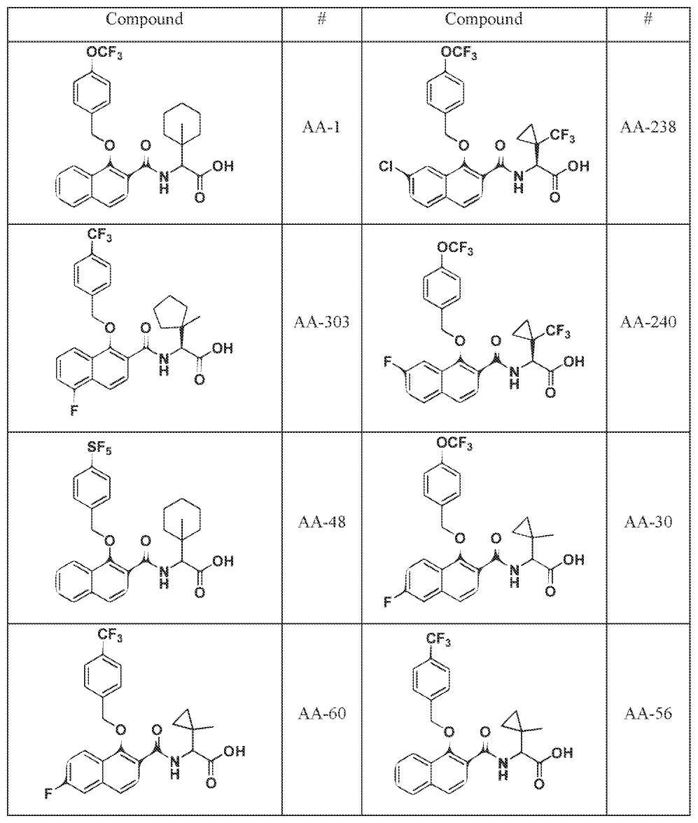

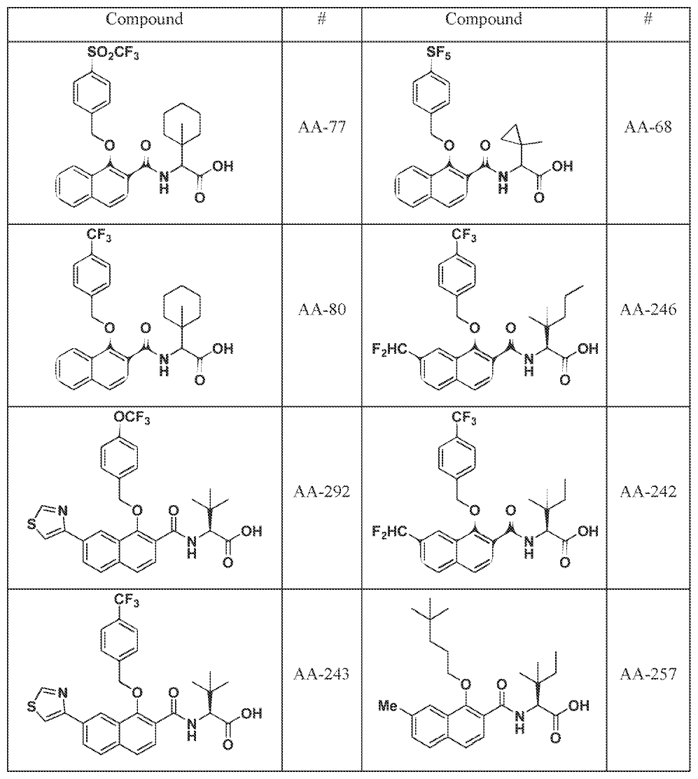

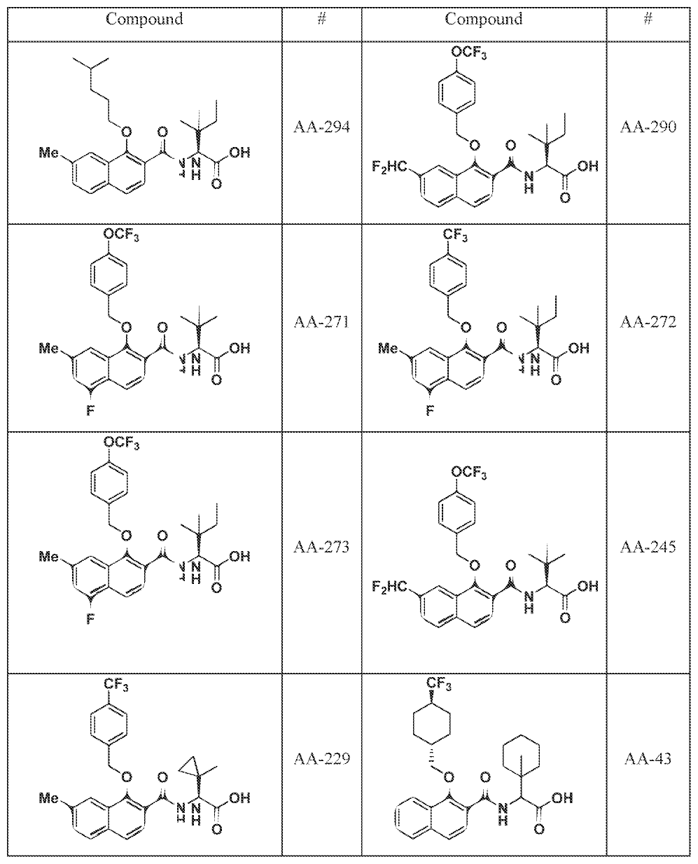

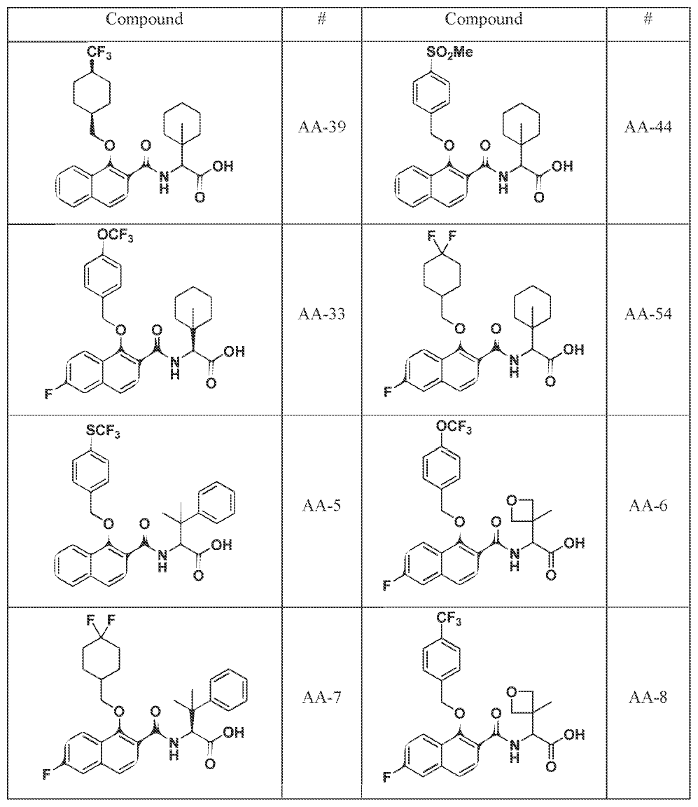

Compounds disclosed in U.S. Patent Application No. 62/944,141 include those of Formula AA below and the compounds shown in FIG. 1.

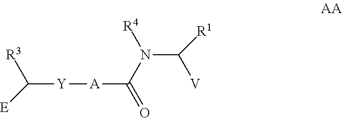

or a pharmaceutically acceptable salt thereof, wherein:
A is

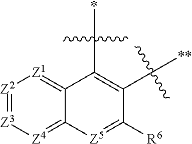

wherein * marks the point of attachment to Y and ** marks the point of attachment to —C(O)—;
Z¹, Z⁴, and Z⁵ are each independently N or CR⁶;
Z² and Z³ are each independently N or CR²;
Y is a bond, —NR³—, —O—, —S—, or —C(R⁴)₂—;
E is C₁₋₆-alkyl, C₂₋₆-alkynyl, C₃₋₉-cycloalkyl, C₄₋₉-cycloalkenyl, C₆₋₁₀-aryl, 3-10 membered heteroaryl, or 3-9 membered heterocycloalkyl, each of which is optionally substituted with one, two, three, four, or five occurrences of R⁵;
V is —C(O)—O—R⁷;
R¹ is

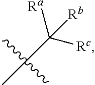

wherein
   R^{a} is hydrogen, halo or C₁₋₆ alkyl,
   R^{b} is hydrogen, halo, C₁₋₆ alkyl, haloC₁₋₆ alkyl, —OH, —CN, halo C₁₋₆ alkyl, thioC₁₋₆ alkyl, C₂₋₆ alkenyl, C₂₋₆ alkynyl, C₆₋₁₀ aryl, —O—C₁₋₆ alkyl, —S—C₁₋₆alkylaryl, —S(O)₂—C₁₋₆alkyl, or —S—C₃₋₉-cycloalkyl, wherein each alkyl, alkenyl, alkynyl, cycloalkyl and aryl is optionally substituted with one, two, three, four, or five occurrences of R⁹;
   R^{c} is C₁₋₆ alkyl, haloC₁₋₆ alkyl, arylC₁₋₆ alkyl, C₃₋₉-cycloalkyl, C₆₋₁₀ aryl, 3-10 membered heteroaryl, or 3-9 membered heterocycloalkyl, each of which is optionally and independently substituted with one, two, three, four, or five occurrences of R¹⁰;
   R^{a} and R^{c}, taken together with the atoms to which they are attached, form a C₃₋₈ cycloalkyl, an aryl or a C₃₋₈ heterocycloalkyl ring, or
   R^{a}, R^{b}, and R^{c} are taken together with the atoms to which they are attached to form a bridged C₅_₉ cycloalkyl;
each R² is independently hydrogen, halo, —CN, —OH, —NH₂, —N—(C₁₋₆ alkyl)₂, C₁₋₆ alkyl, haloC₁₋₆ alkyl, —O—C₁₋₆alkyl, —O-haloC₁₋₆ alkyl, —O—C₂₋₆ alkenyl, or 3-10 membered heteroaryl;
R³ is hydrogen or C₁₋₆ alkyl;
R⁴ is hydrogen or C₁₋₄ alkyl;
each R⁵ is independently halo, —CN, —NO₂, C₁₋₆ alkyl, hydroxyC₁₋₆ alkyl, C₁₋₆ haloalkyl, C₃₋₉-cycloalkyl, C₂₋₆-alkenyl, —O—C₁₋₆alkyl, —O—C₁₋₆haloalkyl, —C(O)—O—C₁₋₆alkyl, —SF₅, —S—C₁₋₆alkyl, —S—C₁₋₆haloalkyl, —S(O)₂—C₁₋₆alkyl, —S(O)₂—C₁₋₆haloalkyl, or 3-10 membered heteroaryl; or two R⁵ moieties, taken together with the atoms to which they are attached, form a 3-8 membered heterocycloalkyl ring, wherein each heterocycloalkyl is substituted with one, two, or three occurrences of R⁸;
each R⁶ is independently hydrogen, halo, or C₁₋₆-alkyl;
R⁷ is hydrogen, C₁₋₆-alkyl, C₆₋₁₀ aryl, or benzyl;
each R⁸ is independently hydrogen, halo or C₁₋₆-alkyl;
each R⁹ is independently hydrogen, halo, C₁₋₆-alkyl, haloC₁₋₆-alkyl, or C₃₋₉-cycloalkyl;
each R¹⁰ is independently hydrogen, halo, —CN, —OH, C₁₋₆-alkyl, haloC₁₋₆ alkyl, —O—C₁₋₆alkyl, —O-haloC₁₋₆ alkyl, —O—C₂₋₆-alkenyl, C₁₋₄-alkylaryl, —O—C₁₋₆-alkylaryl, —O—C₁₋₆alkyl-O—C₁₋₆alkyl, —C(O)OC₁₋₆ alkyl, —S—C₁₋₆alkyl, —N═(NH₂)₂, C₆₋₁₀ aryl, or 3-10 membered heteroaryl; and
m is 0, 1, or 2;
   provided that when R² is hydrogen, R^{a} is hydrogen, and R^{b} is hydrogen or methyl, then R^{c} is not C₁₋₂ alkyl, halo C₁₋₂ alkyl, or 5-membered heteroaryl.

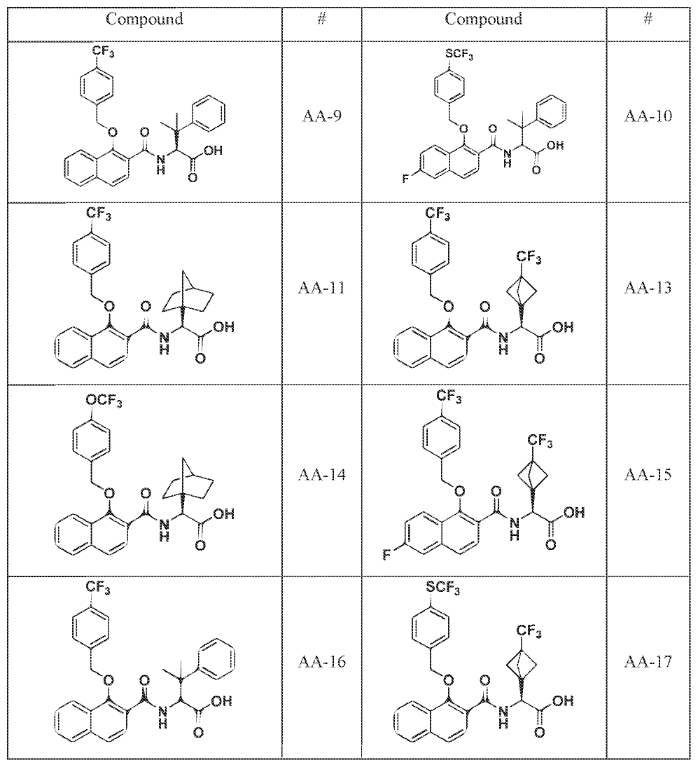

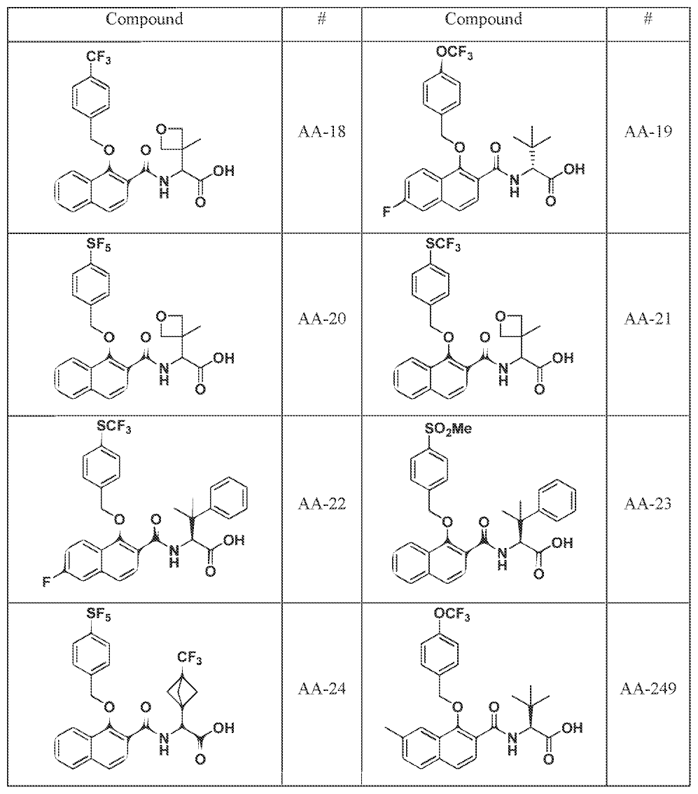

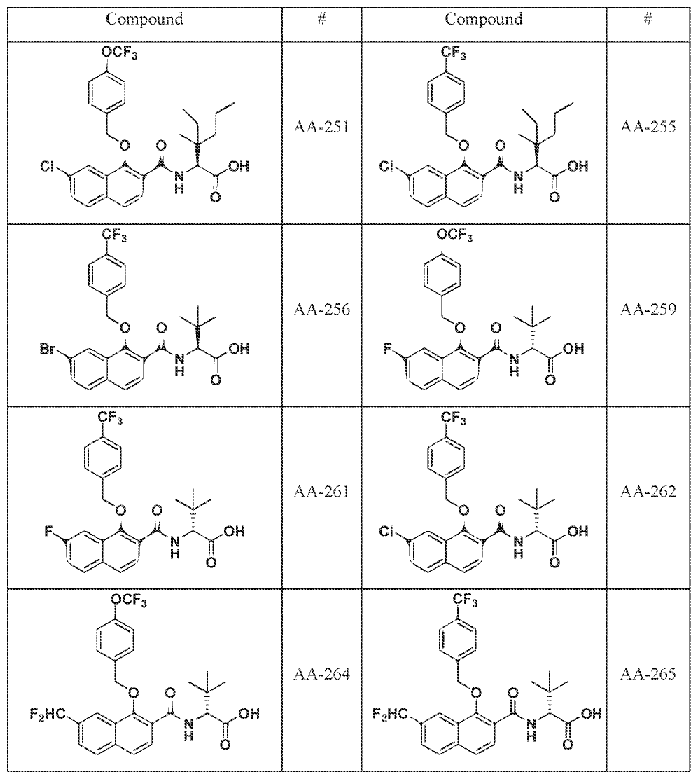

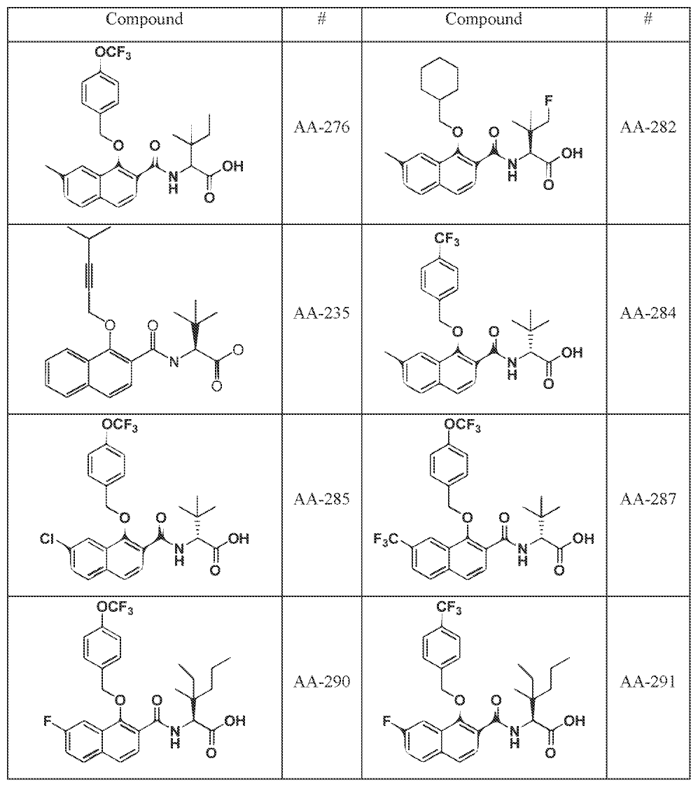

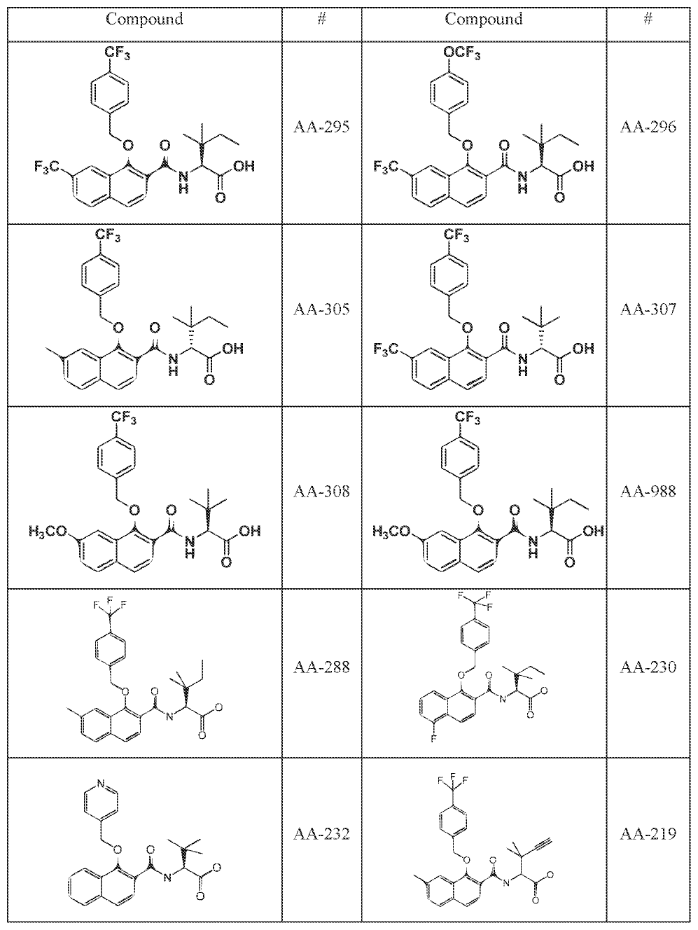

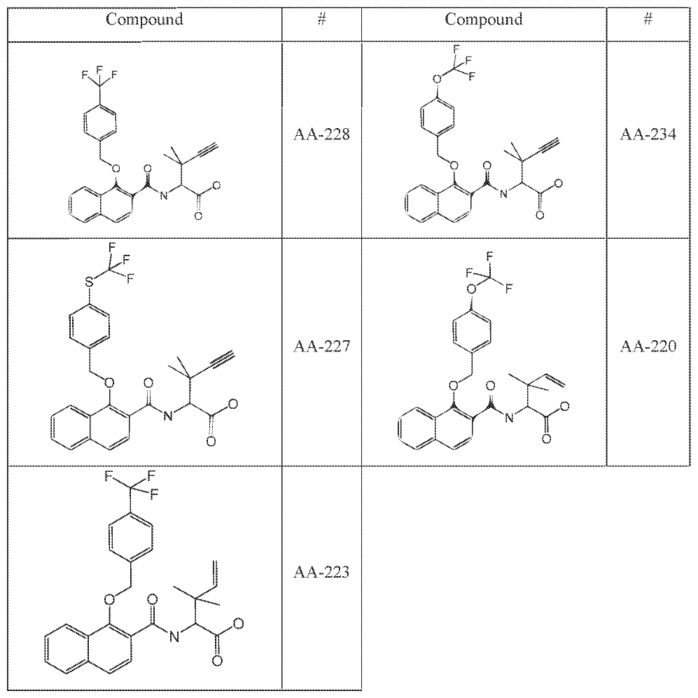

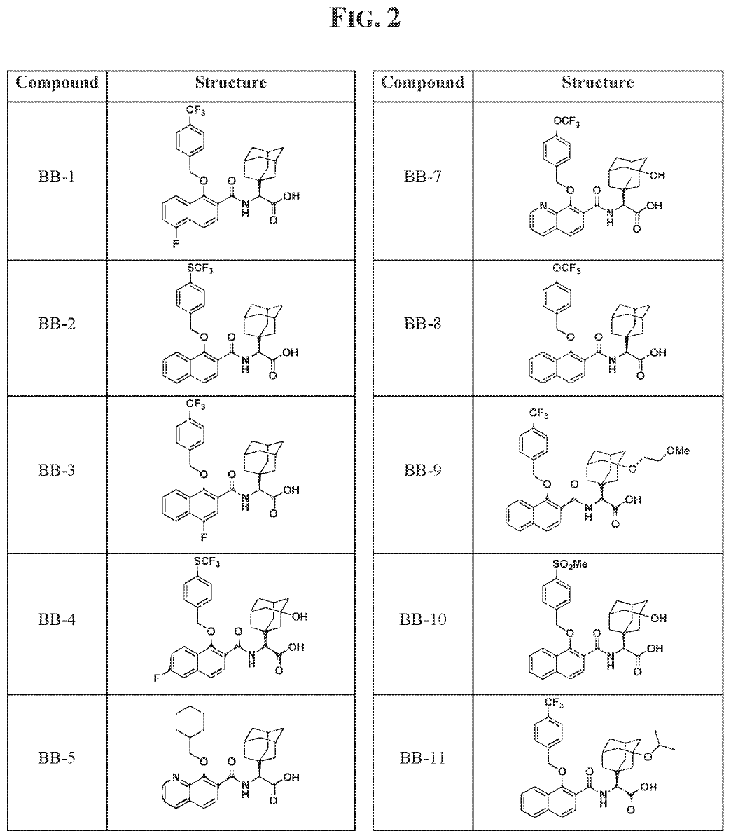

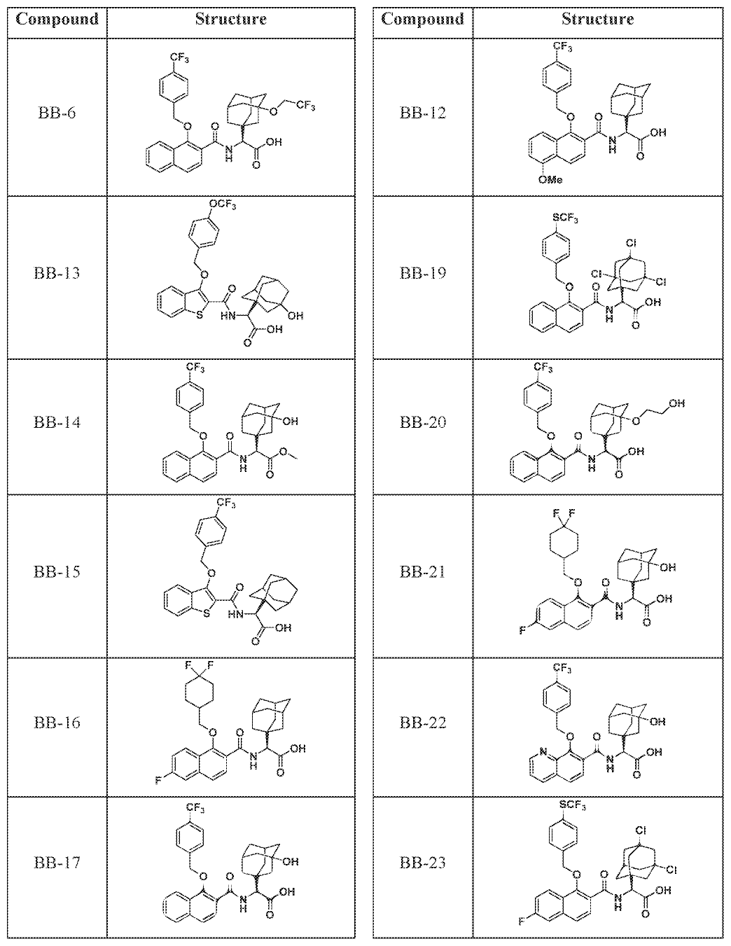

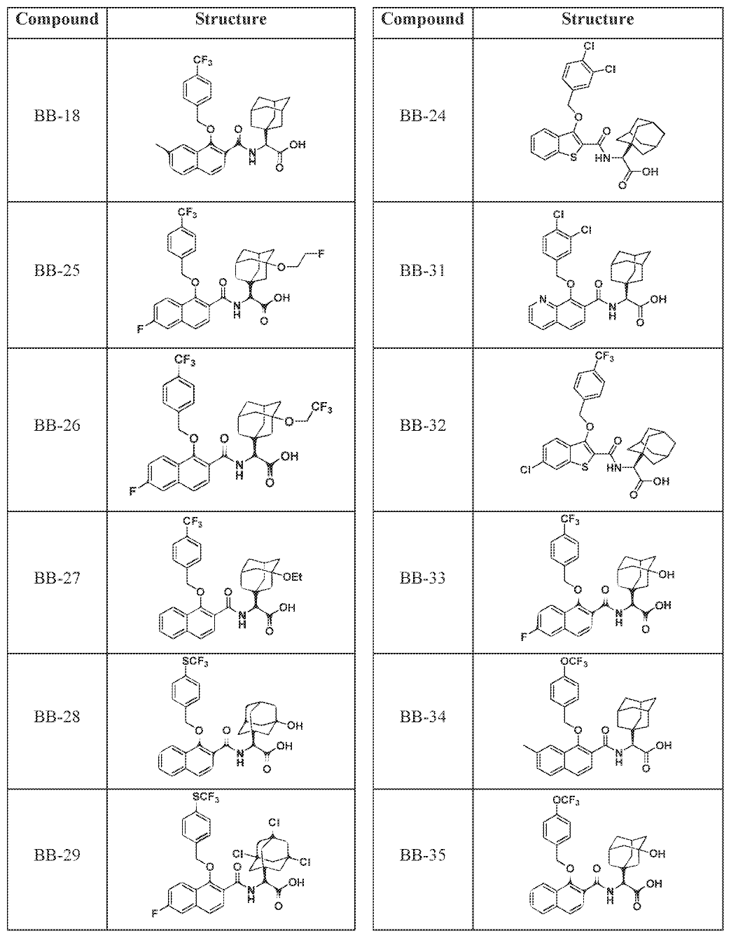

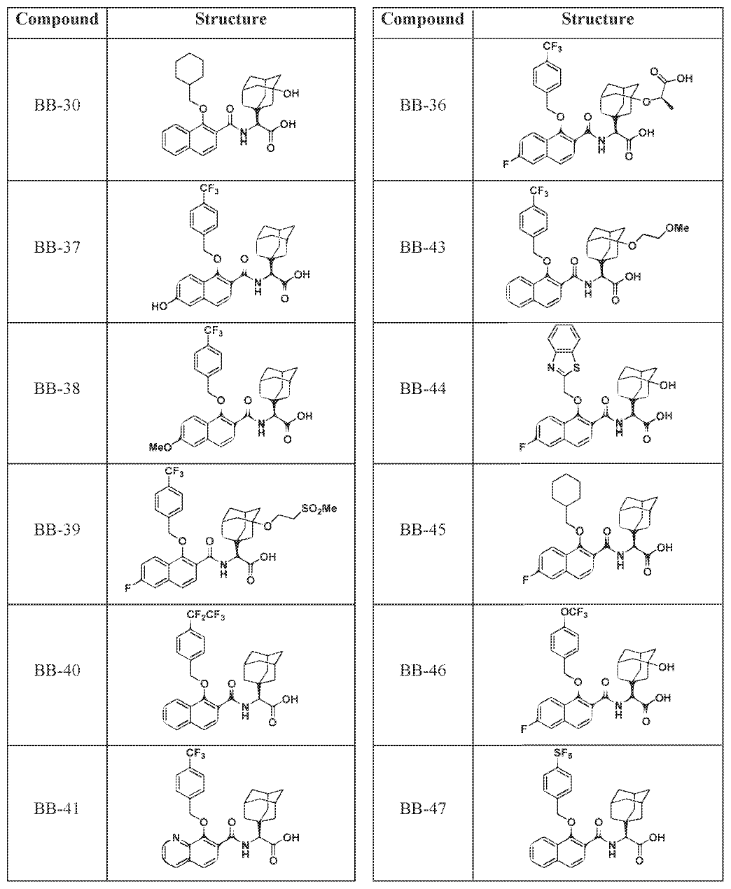

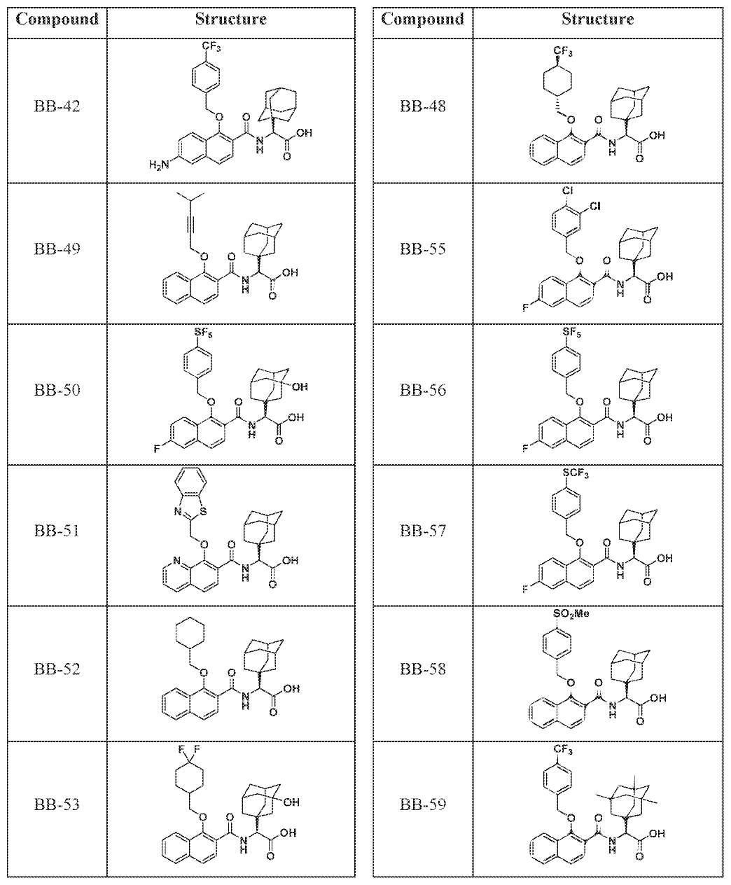

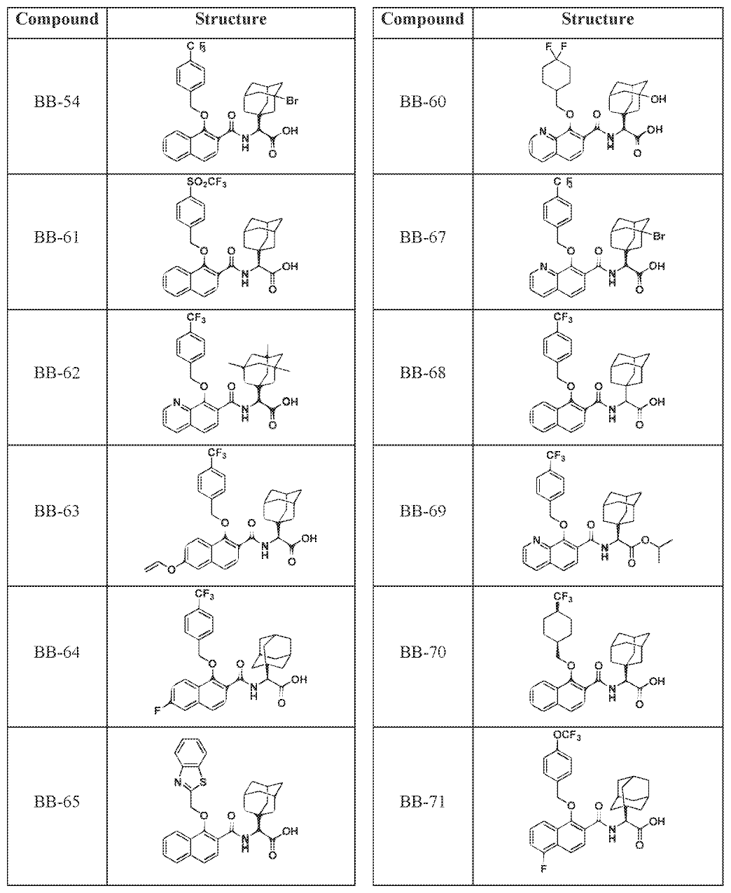

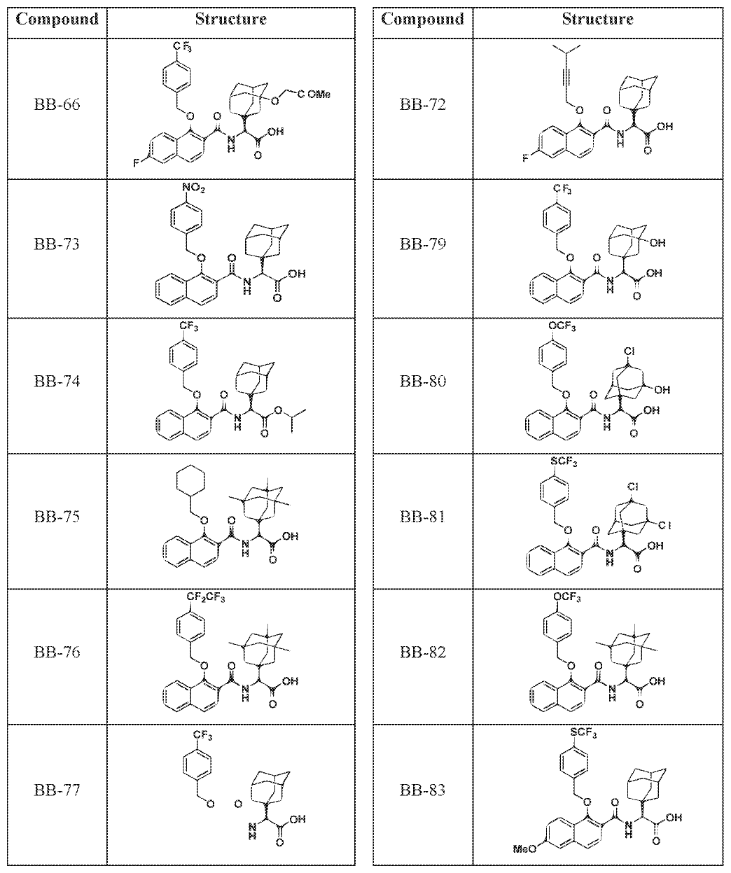

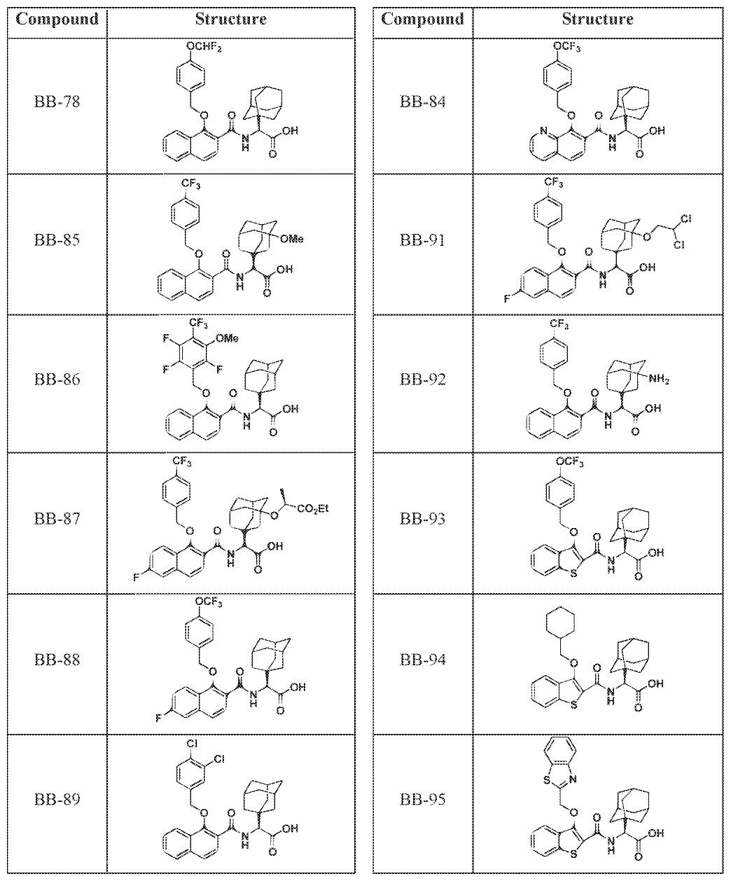

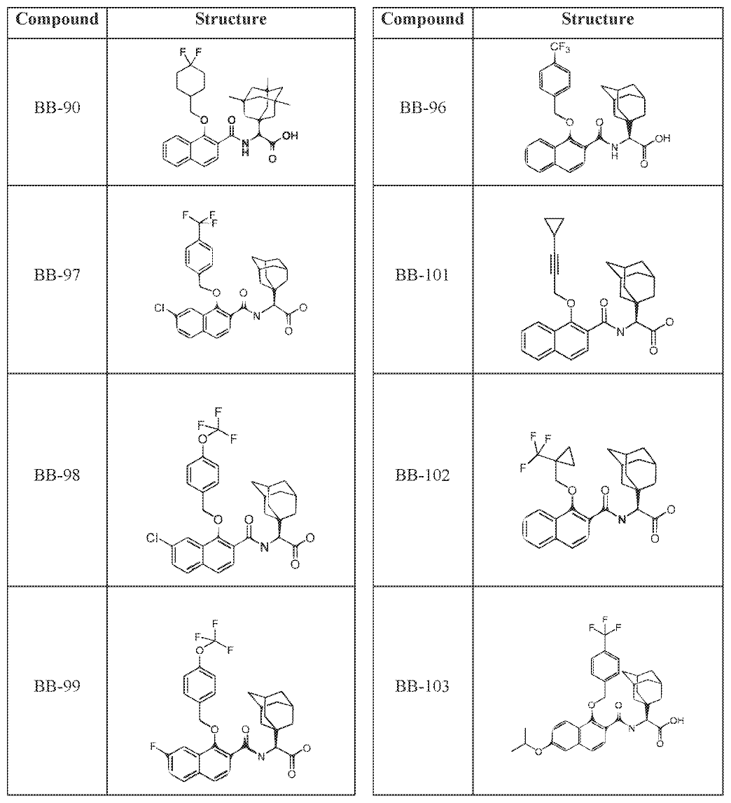

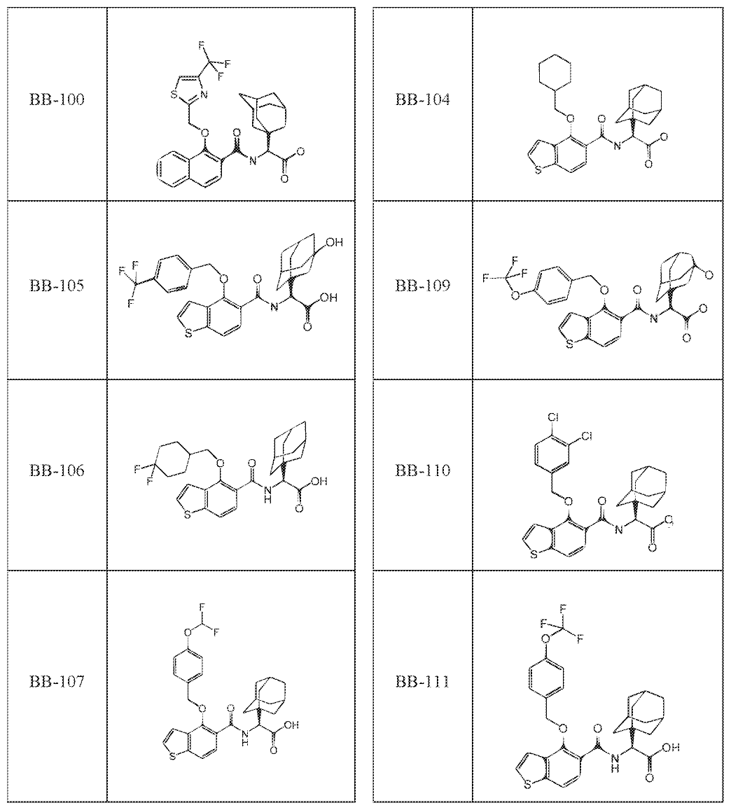

Compounds disclosed in U.S. Patent Application No. 62/944,158 include those of Formula BB below and the compounds shown in FIG. 2.

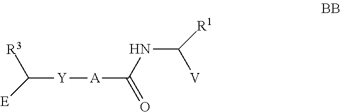

or a pharmaceutically acceptable salt thereof, wherein:
A is selected from

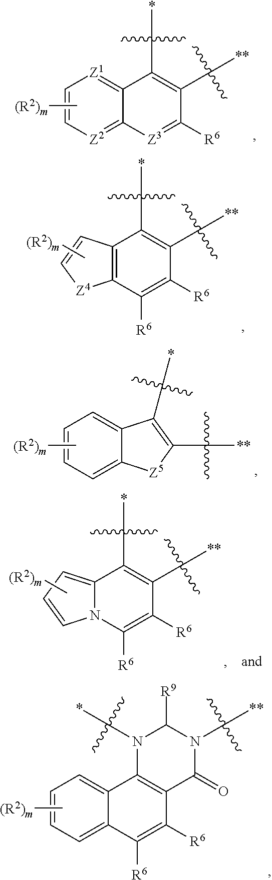

wherein * marks the point of attachment to Y and ** marks the point of attachment to —C(O)—;
Z¹, Z², and Z³ are each independently CR¹⁰ or N;
Z⁴ and Z⁵ are each independently O or S;
Y is a bond, —NR³—, —O—, —S—, or —C(R⁴)₂—;
E is C₂₋₆-alkynyl, C₃₋₉-cycloalkyl, C₄₋₉-cycloalkenyl, C₆₋₁₀-aryl, 3-10 membered heteroaryl, or a 3-9 membered heterocycloalkyl, each of which is optionally substituted with one, two, three, four, or five occurrences of R⁵;
V is —C(O)—O—R⁷;
R¹ is adamantyl optionally substituted with one, two, three, four, or five occurrences of R^{d};
each R^{d} is independently —OH, —NH₂, —N═CH(NH₂)₂, halo, C₁₋₄-alkyl, —O—C₁₋₆-alkyl, —O—C₁₋₆-alkyl-OH, —C₁₋₆-alkyl-OH, —C₁₋₆ haloalkyl, —O—C₁₋₆-haloalkyl, —C₁₋₆-alkyl-OH, —C₁₋₆-alkyl-COOH, —C₁₋₆-alkyl-C(O)₂—C₁₋₆-alkyl, —O—C₁₋₆-alkyl-C(O)—C₁₋₆-alkyl, —O—C₁₋₆-alkyl-O—C₁₋₆-alkyl, —O—C₁₋₆-alkyl-C(O)OH, —O—C₁₋₆-alkyl-C(O)O—C₁₋₆-alkyl, or —O—C₁₋₆-alkyl-S(O)—O—C₁₋₆-alkyl;
each R² is independently halo, —OH, —NH₂, —N—(C₁₋₆-alkyl)₂, C₁₋₆-alkyl, C₁₋₆-haloalkyl, —O—C₁₋₆-alkyl, —O—C₁₋₆-alkyl-OH, —O—C₁₋₆-alkyl-O—C₁₋₆-alkyl, —O—C₂₋₆-alkenyl, or C₆₋₁₀-aryl;
R³ is hydrogen or C₁₋₆ alkyl;
R⁴ is independently hydrogen or C₁₋₆ alkyl;
each R⁵ is independently halo, —CN, —NO₂, —SF₃, —SF₅, C₁₋₆-alkyl, C₂₋₆-alkenyl, C₁₋₆-haloalkyl, —O—C₁₋₆-alkyl, —O—C₁₋₆-haloalkyl, —C₁₋₆-alkyl-OH, —C(O)—O—C₁₋₆-alkyl, —S—C₁₋₆alkyl, —S—C₁₋₆-haloalkyl, —S(O)₂—C₁₋₆-alkyl, —S(O)₂—C₁₋₆-haloalkyl, C₆₋₁₀-aryl, or —C₃₋₉-cycloalkyl; or two R⁵ moieties, taken together with the atoms to which they are attached, form a 3-9 membered heterocycloalkyl ring, wherein each aryl, cycloalkyl, or heterocycloalkyl is substituted with one, two, or three occurrences of R⁸;
each R⁶ and R¹⁰ is independently hydrogen, halo, C₁₋₆-alkyl, or —O—C₁₋₆-alkyl;
R⁷ is hydrogen, C₁₋₆-alkyl, C₆₋₁₀-aryl, or benzyl;
each R⁸ is independently halo or C₁₋₆-alkyl;
R⁹ is hydrogen, C₁₋₆ alkyl or oxo; and
m is 0, 1, or 2.

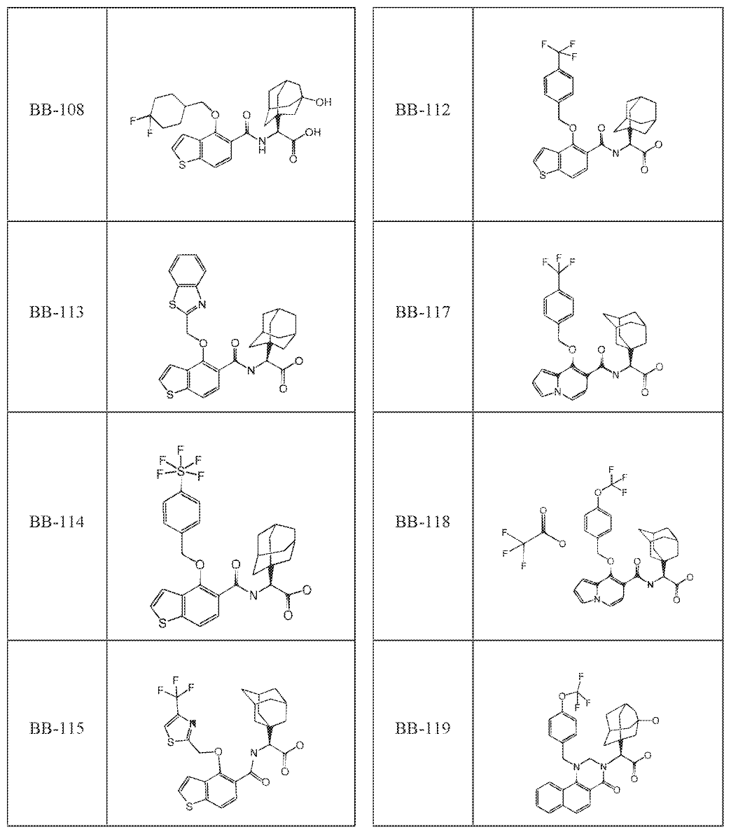

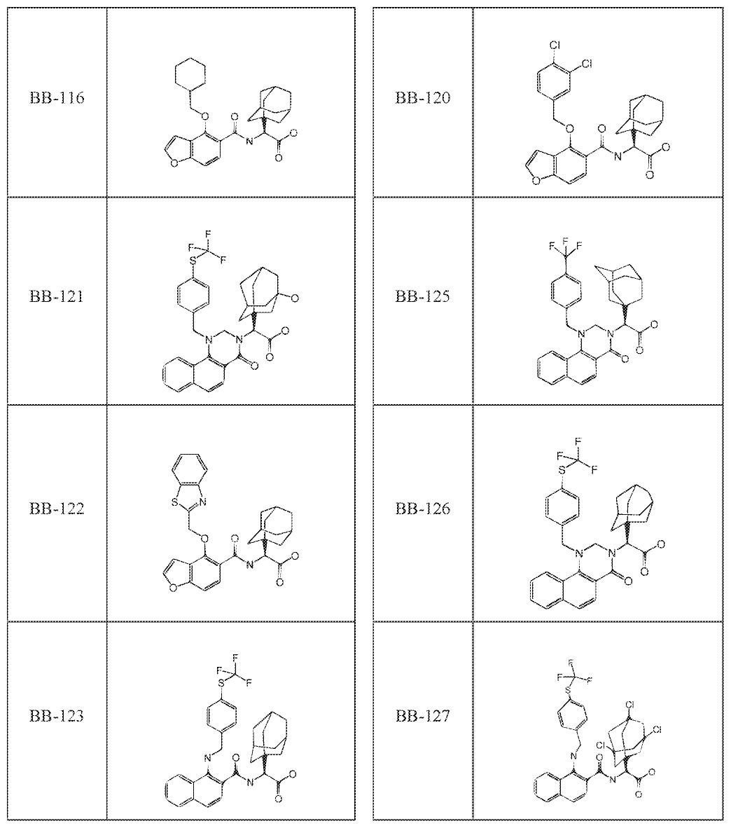

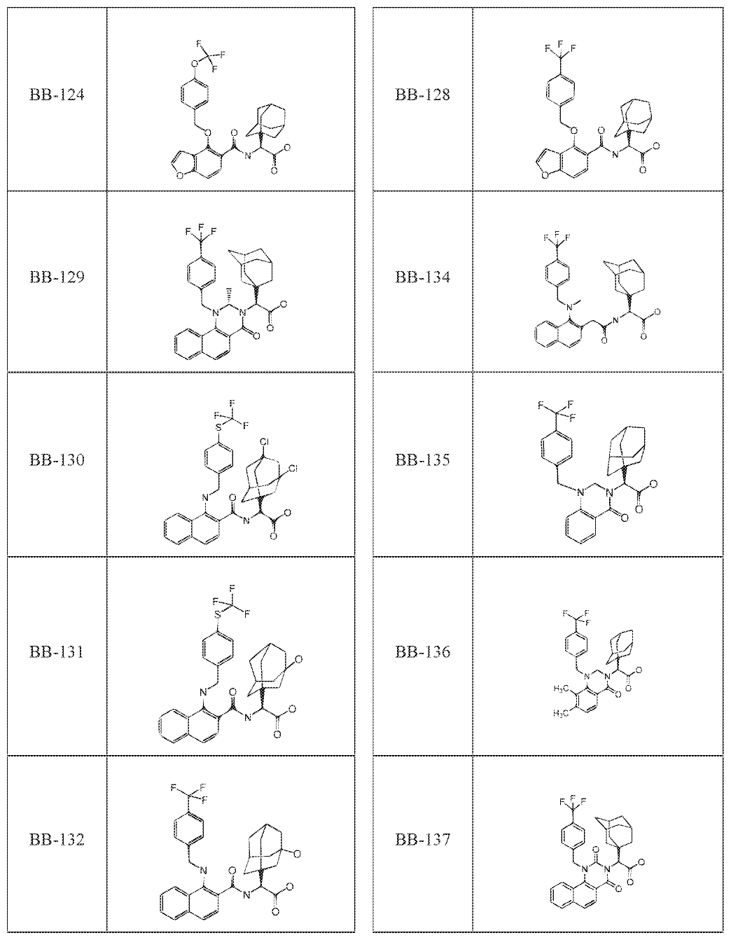

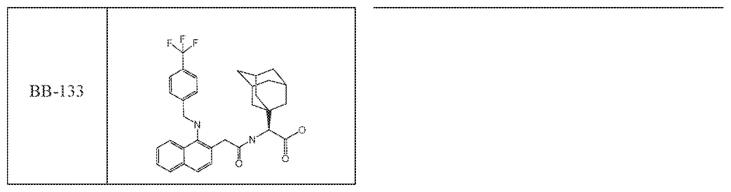

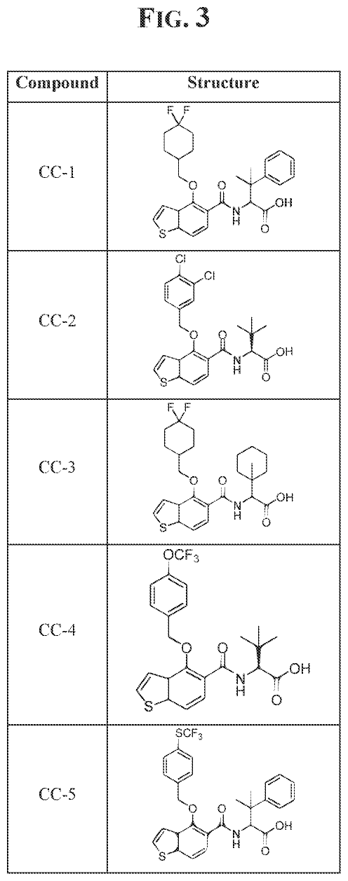

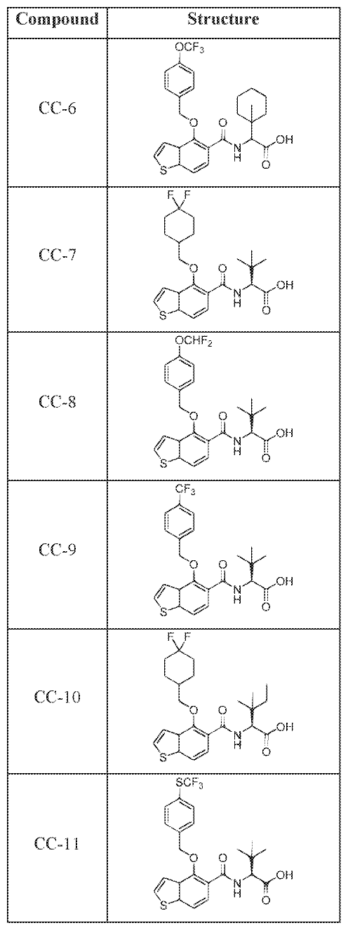

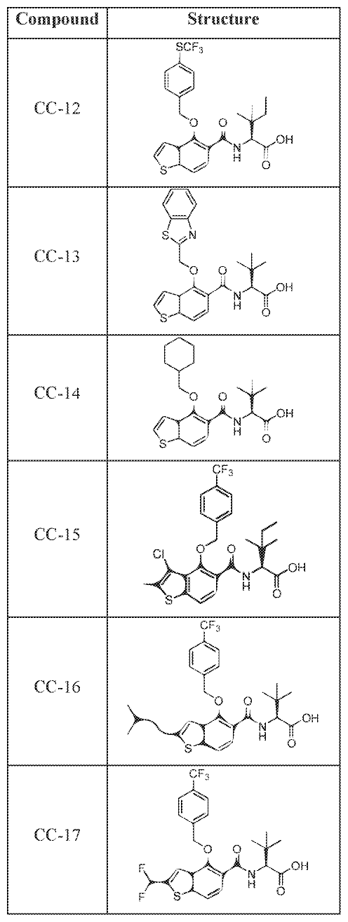

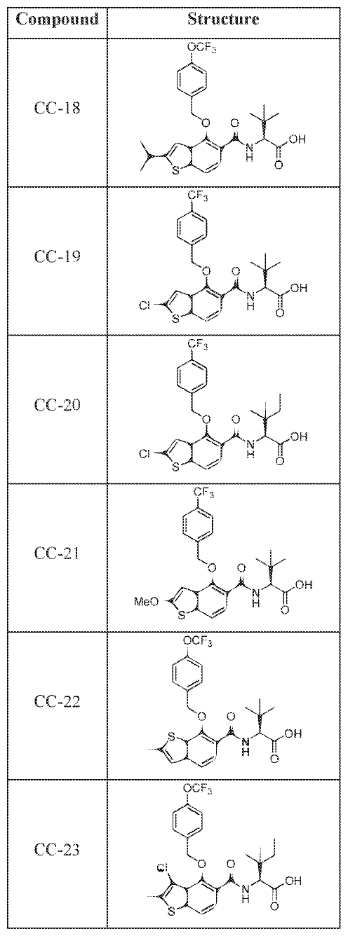

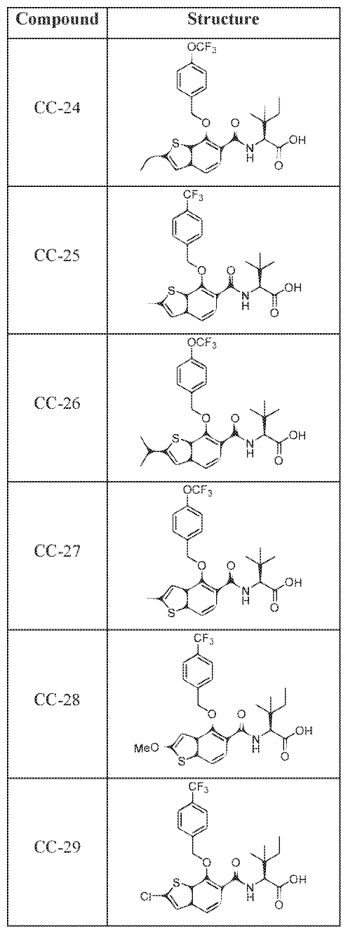

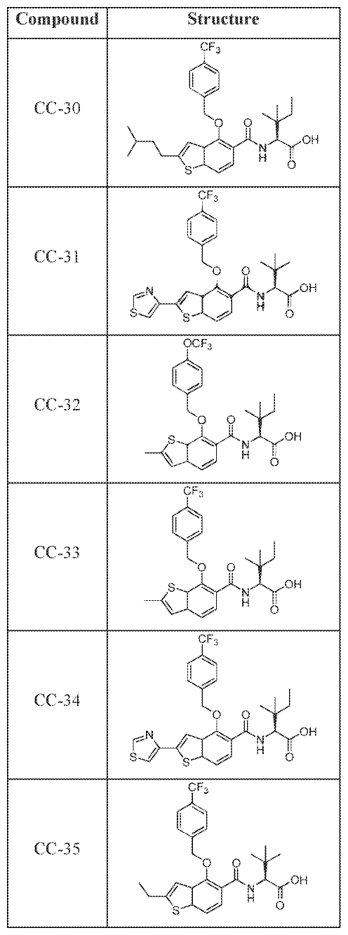

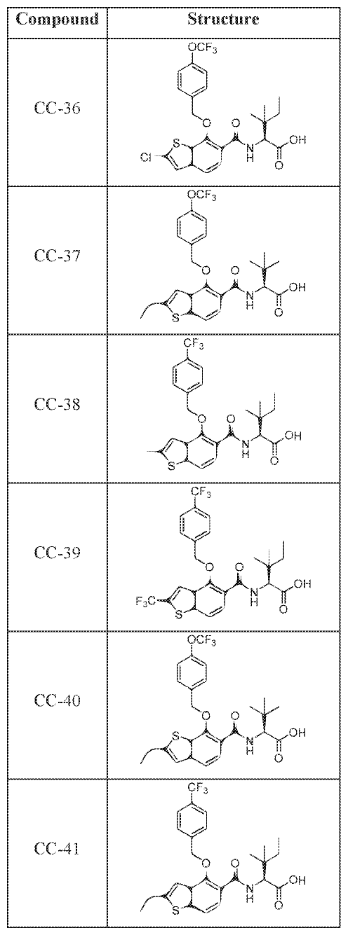

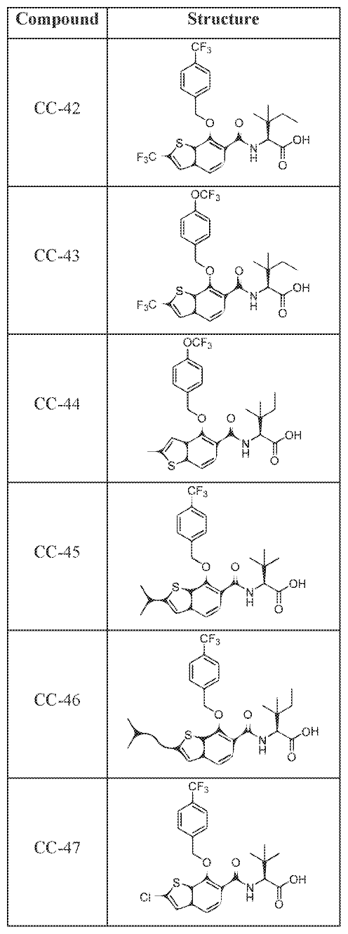

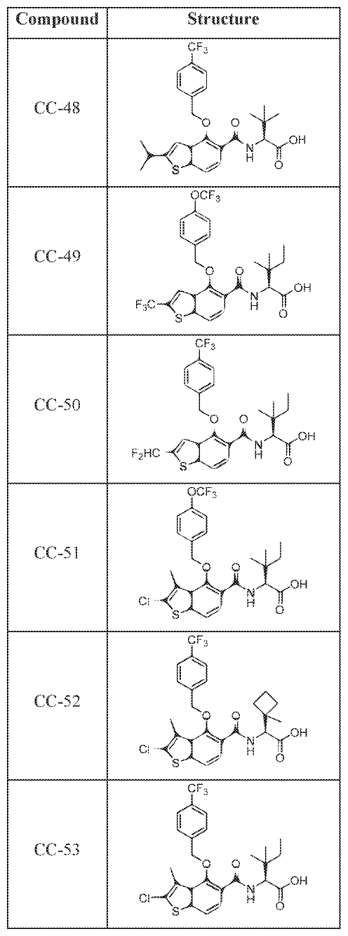

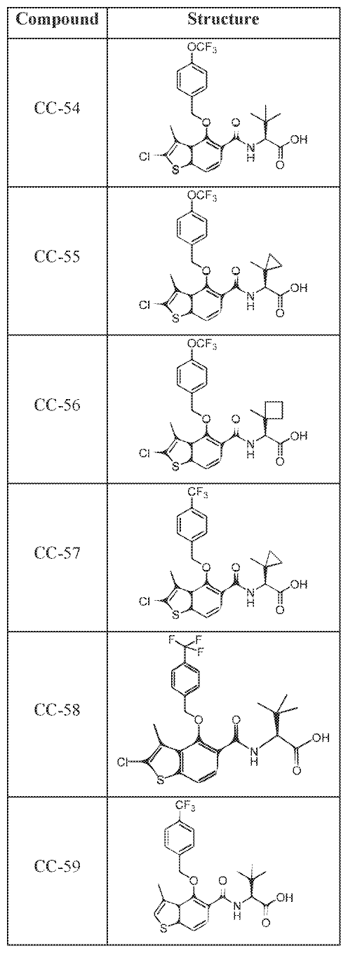

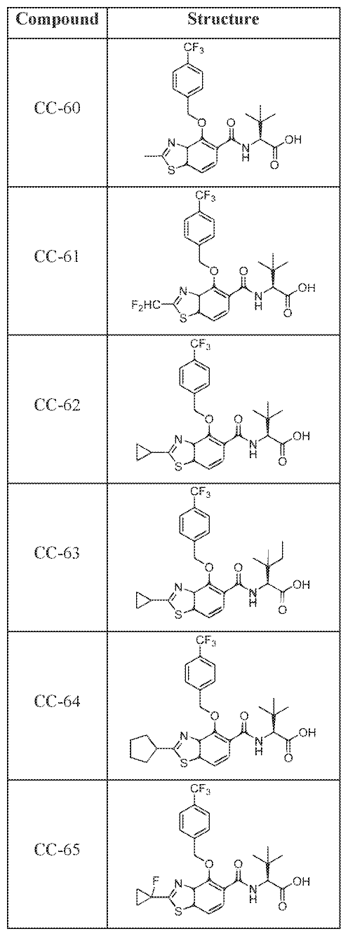

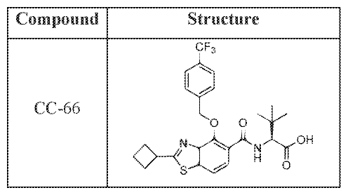

Compounds disclosed in U.S. Patent Application No. 62/944,188 include those of Formula CC below and the compounds shown in FIG. 3.

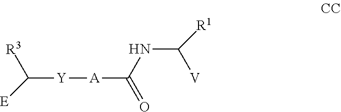

or a pharmaceutically acceptable salt thereof, wherein:
A is selected from

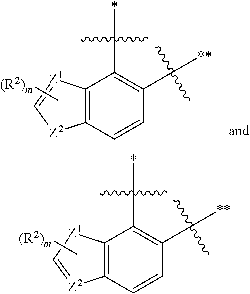

wherein * marks the point of attachment to Y and ** marks the point of attachment to —C(O)—;
Z¹ and Z² are each independently CH, S or N, wherein at least one of Z¹ and Z² is N or S;
Y is —O—;
E is C₃₋₉-cycloalkyl, C₆₋₁₀-aryl, 3-10 membered heteroaryl, or a 3-9 membered heterocycloalkyl, each of which is optionally substituted with one, two, three, four, or five occurrences of R⁵;
V is —C(O)—O—R⁷;
R¹ is

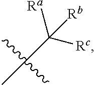

wherein
   R^{a} is C₁₋₆ alkyl,
   R^{b} is C₁₋₆ alkyl,
   R^{c} is C₁₋₆ alkyl or C₆₋₁₀ aryl, or
   any two of R^{a}, R^{b}, and R^{c}, taken together with the atoms to which they are attached, form a C₃₋₉ cycloalkyl ring;
each R² is independently halo, C₁₋₆ alkyl, haloC₁₋₆ alkyl, —O—C₁₋₆ alkyl, —O-haloC₁_6 alkyl, C₃₋₉-cycloalkyl or 3-10 membered heteroaryl;
R³ is H or alkyl;
each R⁵ is independently halo, C₁₋₆ alkyl, C₁₋₆ haloalkyl, —O—C₁₋₆alkyl, —O—C₁₋₆haloalkyl, —S—C₁₋₆alkyl and —S—C₁₋₆haloalkyl;
R⁷ is hydrogen, C₁₋₆-alkyl, C₆₋₁₀-aryl, or benzyl; and
m is 0, 1, or 2.

Non-limiting examples of anti-inflammatory agents are N6022 (3-(5-(4-(IH-imidazol-I-yl)10 phenyl)-I-(4-carbamoyl-2-methylphenyl)-′H-pyrrol-2-yl) propanoic acid), Ibuprofen, Lenabasum (anabasum), Acebilustat (CTX-4430), LAU-7b, POL6014, docosahexaenoic acid, alpha-1 anti-trypsin, sildenafil. Additional therapeutic agents also include, but are not limited to a mucolytic agent, a modifier of mucus rheology (such as hypertonic saline, mannitol, and oligosaccharide based therapy), a bronchodialator, an anti-infective (such as tazobactam, piperacillin, rifampin, meropenum, ceftazidime, aztreonam, tobramycin, fosfomycin, azithromycin, amitriptyline (?), vancomycin, gallium and colistin), an anti-infective agent, an anti-inflammatory agent, a CFTR modulator other than a compound of the present invention, and a nutritional agent. Additional therapeutic agents can include treatments for comorbid conditions of cyctic fibrosis, such as exocrine pancreatic insufficiency which can be treated with Pancrelipase or Liprotamase.

Examples of CFTR potentiators include, but are not limited to, Ivacaftor (VX-770), CTP-656, NVS-QBW251, FD1860293. GLPG2451, GLPG1837, and N-(3-carbamoyl-5,5,7,7-tetramethyl-5,7-dihydro-4H-thieno[2,3-e]pyran-2-yl)-1H-pyrazole-5-carboxamide. Examples of potentiators are also disclosed in publications: WO2005120497, WO2008147952, WO2009076593, WO2010048573. WO2006002421, WO2008147952, WO2011072241, WO2011113894, WO2013038373, WO2013038378, WO2013038381, WO2013038386, WO2013038390, WO2014180562, WO2015018823, and U.S. patent application Ser. Nos. 14/271,080, 14/451,619 and 15/164,317.

Non-limiting examples of correctors include Lumacaftor (VX-809), 1-(2,2-difluoro-1,3-benzodioxol-5-yl)-N-{1-[(2R)-2,3-dihydroxypropyl]-6-fluoro-2-(1-hydroxy-2-methylpropan-2-yl)-1H-indol-5-yl}cyclopropanec arboxamide (VX-661), VX-983, GLPG2222, GLPG2665, GLPG2737, VX-152, VX-440. FDL169. FDL304, FD2052160, and FD2035659. Examples of correctors are also disclosed in US20160095858A1, and U.S. application Ser. Nos. 14/925,649 and 14/926,727.

In certain embodiments, the additional therapeutic agent is a CFTR amplifier. CFTR amplifiers enhance the effect of known CFTR modulators, such as potentiators and correctors. Examples of CFTR amplifier include PTI130 and PTI-428. Examples of amplifiers are also disclosed in publications: WO2015138909 and WO2015138934.

In certain embodiments, the additional therapeutic agent is an agent that reduces the activity of the epithelial sodium channel blocker (ENaC) either directly by blocking the channel or indirectly by modulation of proteases that lead to an increase in ENaC activity (e.g., serine proteases, channel-activating proteases). Exemplary of such agents include camostat (a trypsin-like protease inhibitor), QAU145, 552-02, GS-9411, INO-4995, Aerolytic, amiloride, AZD5634, and VX-371. Additional agents that reduce the activity of the epithelial sodium channel blocker (ENaC) can be found, for example, in PCT Publication No. WO2009074575 and WO2013043720; and U.S. Pat. No. 8,999,976.

In one embodiment, the ENaC inhibitor is VX-371.

In one embodiment, the ENaC inhibitor is SPX-101 (S18).

In certain embodiments, the combination of a compound of Formula I, with a second therapeutic agent may have a synergistic effect in the treatment of cancer and other diseases or disorders mediated by adenosine. In other embodiments, the combination may have an additive effect.

### Pharmaceutical Compositions

The compositions and methods of the present invention may be utilized to treat a subject in need thereof. In certain embodiments, the subject is a mammal such as a human, or a non-human mammal. When administered to subject, such as a human, the composition or the compound is preferably administered as a pharmaceutical composition comprising, for example, a compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In a preferred embodiment, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as an eye drop.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-microemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of a subject without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the subject. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); anally, rectally or vaginally (for example, as a pessary, cream or foam); parenterally (including intramuscularly, intravenously, subcutaneously or intrathecally as, for example, a sterile solution or suspension); nasally; intraperitoneally; subcutaneously; transdermally (for example as a patch applied to the skin); and topically (for example, as a cream, ointment or spray applied to the skin, or as an eye drop). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the subject being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions for rectal, vaginal, or urethral administration may be presented as a suppository, which may be prepared by mixing one or more active compounds with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the pharmaceutical compositions for administration to the mouth may be presented as a mouthwash, or an oral spray, or an oral ointment.

Alternatively or additionally, compositions can be formulated for delivery via a catheter, stent, wire, or other intraluminal device. Delivery via such devices may be especially useful for delivery to the bladder, urethra, ureter, rectum, or intestine.

Formulations which are suitable for vaginal administration also include pessaries, tampons, creams, gels, pastes, foams or spray formulations containing such carriers as are known in the art to be appropriate.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention. Exemplary ophthalmic formulations are described in U.S. Publication Nos. 2005/0080056, 2005/0059744, 2005/0031697 and 2005/004074 and U.S. Pat. No. 6,583,124, the contents of which are incorporated herein by reference. If desired, liquid ophthalmic formulations have properties similar to that of lacrimal fluids, aqueous humor or vitreous humor or are compatible with such fluids. A preferred route of administration is local administration (e.g., topical administration, such as eye drops, or administration via an implant).

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinacious biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the subject being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the subject's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In preferred embodiments, the active compound will be administered once daily.

In certain embodiments, the dosing follows a 3+3 design. The traditional 3+3 design requires no modeling of the dose-toxicity curve beyond the classical assumption for cytotoxic drugs that toxicity increases with dose. This rule-based design proceeds with cohorts of three patients; the first cohort is treated at a starting dose that is considered to be safe based on extrapolation from animal toxicological data, and the subsequent cohorts are treated at increasing dose levels that have been fixed in advance. In some embodiments, the three doses of a compound of formula I range from about 100 mg to about 1000 mg orally, such as about 200 mg to about 800 mg, such as about 400 mg to about 700 mg, such as about 100 mg to about 400 mg, such as about 500 mg to about 1000 mg, and further such as about 500 mg to about 600 mg. Dosing can be three times a day when taken with without food, or twice a day when taken with food. In certain embodiments, the three doses of a compound of formula I range from about 400 mg to about 800 mg, such as about 400 mg to about 700 mg, such as about 500 mg to about 800 mg, and further such as about 500 mg to about 600 mg twice a day. In certain preferred embodiments, a dose of greater than about 600 mg is dosed twice a day.

If none of the three patients in a cohort experiences a dose-limiting toxicity, another three patients will be treated at the next higher dose level. However, if one of the first three patients experiences a dose-limiting toxicity, three more patients will be treated at the same dose level. The dose escalation continues until at least two patients among a cohort of three to six patients experience dose-limiting toxicities (i.e., ≥about 33% of patients with a dose-limiting toxicity at that dose level). The recommended dose for phase II trials is conventionally defined as the dose level just below this toxic dose level.

In certain embodiments, the dosing schedule can be about 40 mg/m² to about 100 mg/m², such as about 50 mg/m² to about 80 mg/m², and further such as about 70 mg/m² to about 90 mg/m² by IV for 3 weeks of a 4 week cycle.

In certain embodiments, compounds of the invention may be used alone or conjointly administered with another type of therapeutic agent. As used herein, the phrase “conjoint administration” refers to any form of administration of two or more different therapeutic compounds such that the second compound is administered while the previously administered therapeutic compound is still effective in the body (e.g., the two compounds are simultaneously effective in the subject, which may include synergistic effects of the two compounds). For example, the different therapeutic compounds can be administered either in the same formulation or in a separate formulation, either concomitantly or sequentially. In certain embodiments, the different therapeutic compounds can be administered within one hour, 12 hours, 24 hours, 36 hours, 48 hours, 72 hours, or a week of one another. Thus, a subject who receives such treatment can benefit from a combined effect of different therapeutic compounds.

In certain embodiments, conjoint administration of compounds of the invention with one or more additional therapeutic agent(s) (e.g., one or more additional chemotherapeutic agent(s)) provides improved efficacy relative to each individual administration of the compound of the invention (e.g., compound of formula I) or the one or more additional therapeutic agent(s). In certain such embodiments, the conjoint administration provides an additive effect, wherein an additive effect refers to the sum of each of the effects of individual administration of the compound of the invention and the one or more additional therapeutic agent(s).

This invention includes the use of pharmaceutically acceptable salts of compounds of the invention in the compositions and methods of the present invention. A salt of a compound of this invention is formed between an acid and a basic group of the compound, such as an amino functional group, or a base and an acidic group of the compound, such as a carboxyl functional group. According to another embodiment, the compound is a pharmaceutically acceptable acid addition salt.

A “pharmaceutically acceptable salt” means any non-toxic salt that, upon administration to a recipient, is capable of providing, either directly or indirectly, a compound of this invention. A “pharmaceutically acceptable counterion” is an ionic portion of a salt that is not toxic when released from the salt upon administration to a recipient.

Acids commonly employed to form pharmaceutically acceptable salts include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, (3-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid.

In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn or other metal salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

Although specific embodiments of the present disclosure will now be described with reference to the preparations and schemes, it should be understood that such embodiments are by way of example only and merely illustrative of but a small number of the many possible specific embodiments which can represent applications of the principles of the present disclosure. Various changes and modifications will be obvious to those of skill in the art given the benefit of the present disclosure and are deemed to be within the spirit and scope of the present disclosure as further defined in the appended claims.

Unless defined otherwise, all technical and scientific terms used herein have the same meaning as commonly understood by one having ordinary skill in the art to which this disclosure belongs. Although other compounds or methods can be used in practice or testing, certain preferred methods are now described in the context of the following preparations and schemes.

A number of synthetic protocols were used to produce the compounds described herein. These synthetic protocols (see schemes below) have common intersections and can be used alternatively for synthesis of the compounds described herein.

### EXAMPLES

### General Schemes

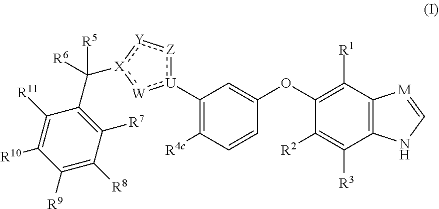

All the substituents and articles are defined as in the general description.

The compounds of the present disclosure can be better understood in connection with the following synthetic schemes and methods which illustrate a means by which the compounds of the formula (I) can be prepared. The compounds of this disclosure can be prepared by a variety of synthetic procedures illustrated in Scheme I to IX.

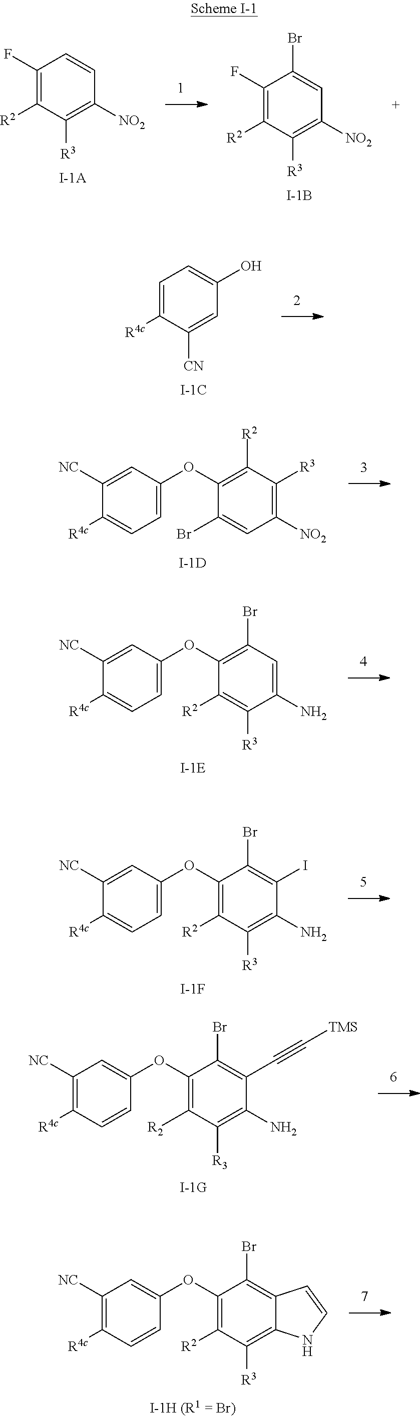

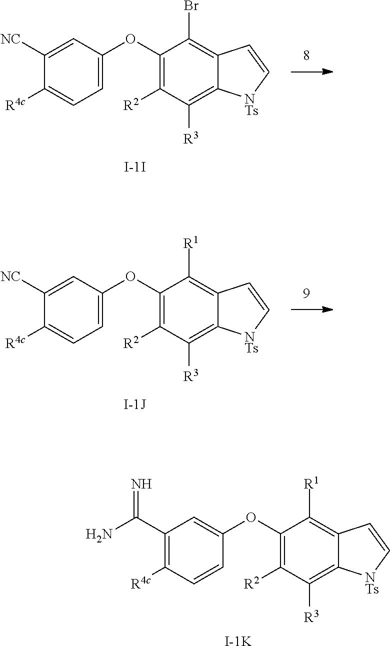

In Scheme I-1, step 1, the properly substituted nitro-benzene (I-1A) is brominated to form bromide I-1B. Intermediate bromide I-1B can be condensed with phenol I-1C to yield intermediate I-1D (step 2). Reduction of the nitro group into amine (step 3) to obtain I-1E, to which iodine is introduced (step 4) to give an intermediate I-1F which is coupled with protected acetylene yielding intermediate I-1G (step 5). Cyclization of I-1G (step 6) yields the key intermediate I-1H. After proper protection of indole (for example, use TsCl to form tosyl) into I-1I. The bromine at C4 in the compound of formula I-1I can be functionalized into R1 (step 8) to different coupling intermediate I-1J. The intermediate I-1J is converted into amidine I-1K.

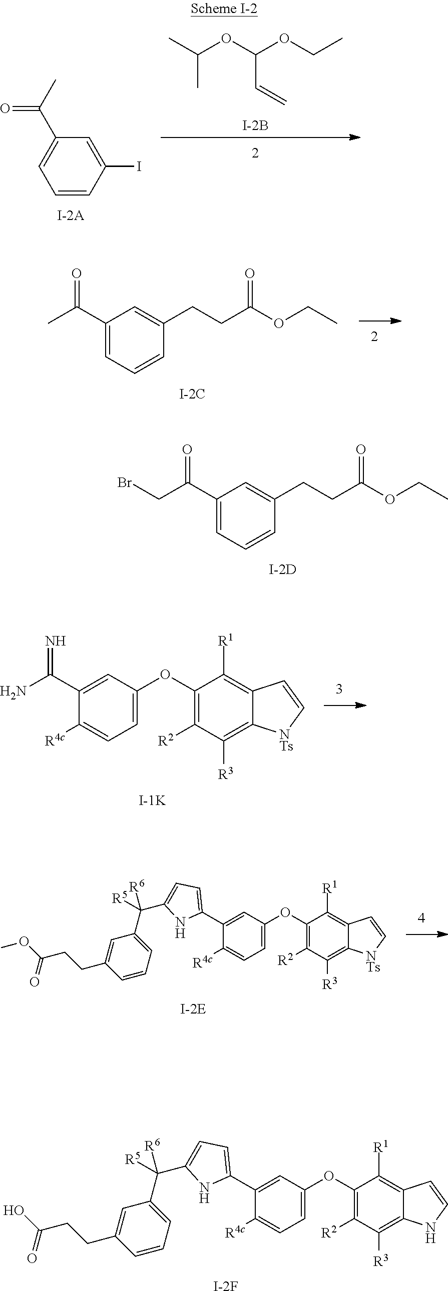

In Scheme I-2, the substituted iodo compound I-2A is coupled with I-2B (step 1) to give I-2C. Bromination (step 2) to give bromo-ketone I-2D. Condensation of I-1K with I-2D (step 3) gives imidazole I-2E. After removal of the protection groups, compound I-2F is obtained.

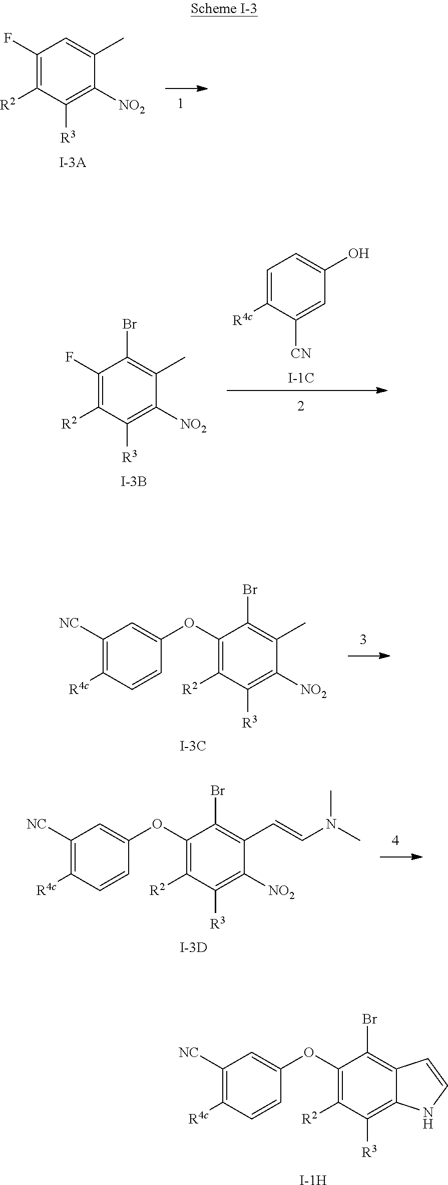

The intermediate I-1H may also be prepared as illustrated in Scheme I-3. Properly substituted methyl nitrobenzene (I-3A) is brominated (step 1) to give bromide I-3B. Compound I-3B is condensed with phenol I-1C (step 2) to give I-3C. Condensation of I-3C with DMF-DMA (step 3) gives I-3D which can be cyclized (step 4) to give I-1H.

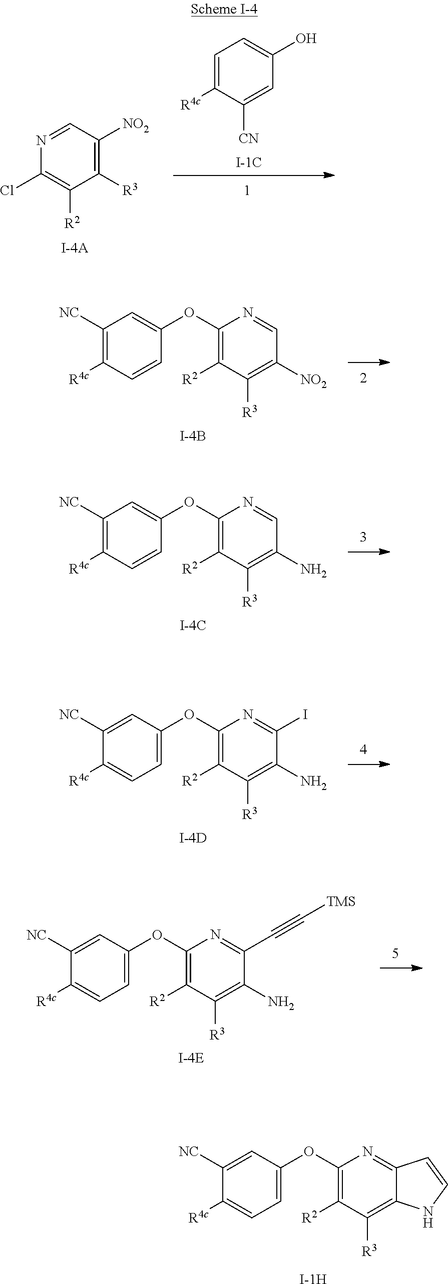

The intermediate I-1H may be prepared according to Scheme I-4. Properly substituted chloro-pyridine I-4A is condensed with I-1C (step 1) to give I-4B. Reduction of the nitro I-4B (step 2) yields amine I-4C. Introduction of iodine to I-4C (step 3) gives rise to I-4D. Coupling of I-4D with protected acetylene (step 4) to obtain I-4E which is cyclized (step 5) to intermediate I-1H.

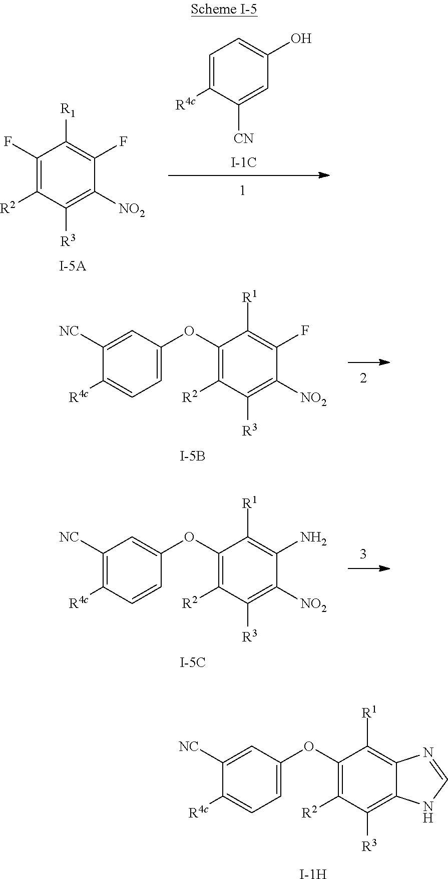

The intermediate I-1H (M=N) can be synthesized according to Scheme I-4. Substituted nitrobenzene I-5A is condensed with I-1C (step 1) to form I-5B. The fluoro is replaced with amine to give I-5C. Nitro aminobenzene I-5C is reduced and cyclized in one step (step 3) to give intermediate I-1H (M=N).

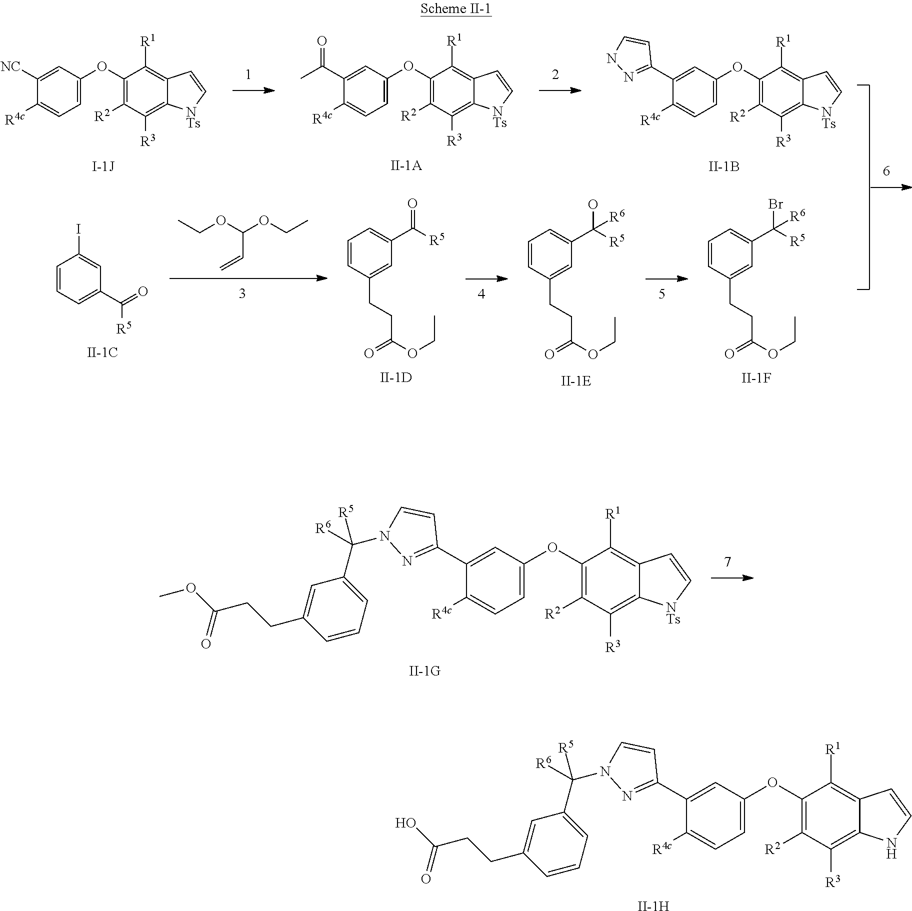

Scheme II-1 illustrates the preparation of the pyrazole analogs II-1H. Intermediate I-1J is treated with methyl magnesium bromide (step 1) to obtain a ketone II-1A. The ketone II-1A is treated with hydrazine (step 2) to form pyrazole II-1B. Properly substituted iodide II-1C is condensed with 3,3-diethoxyprop-1-ene (step 3) to give II-1D. The ketone II-1D is functionalized either by reduction (step 4) to give II-1E (R⁶═H) or by adding Grignard or lithium reagent to obtain II-1E (R⁶=alkyl). The hydroxyl group of II-1E can be transformed (step 5) into bromide II-1F.

Coupling of II-1F with II-1B (step 6) yields II-1G. Removal of the protection groups of II-1G (step 7) yields compound II-1H.

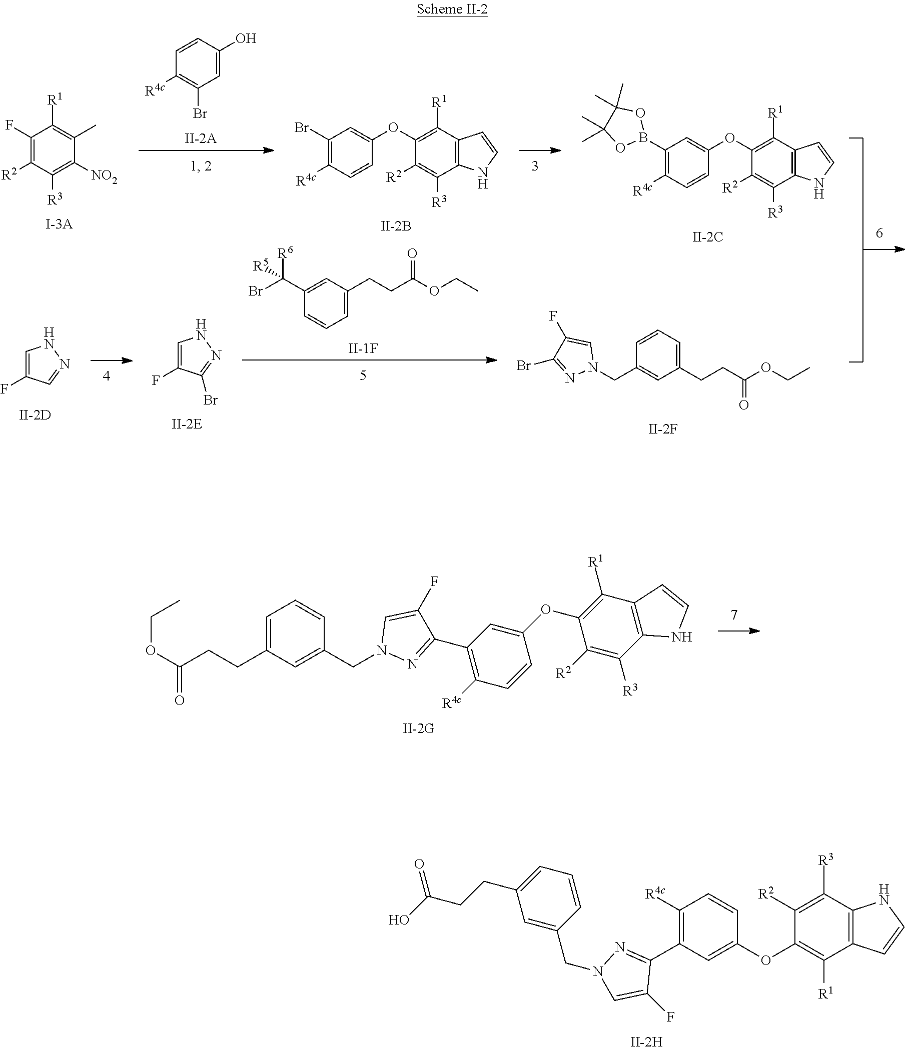

Scheme II-2 illustrates an exemplary route for synthesis of compound II-2H. Starting material I-3A is condensed with II-2A (step 1) to form a diarylether. The diarylether is converted into indole II-2B (step 2) by following the protocol detailed in Scheme I-2, step 3 and 4. The bromide II-2B is transformed (step 3) into boronic ester II-2C. Starting material II-2D is protected, brominated, and deprotected (step 4) to give pyrazole bromide II-2E. Coupling of pyrazole bromide II-2E with bromide II-1F (step 5) produces II-2F. The regio-isomers are isolated via a column separation. Condensation of boronic ester II-2C with pyrazole II-2F (step 6) yields II-2G. After removal of the protecting group, compound II-2H is obtained.

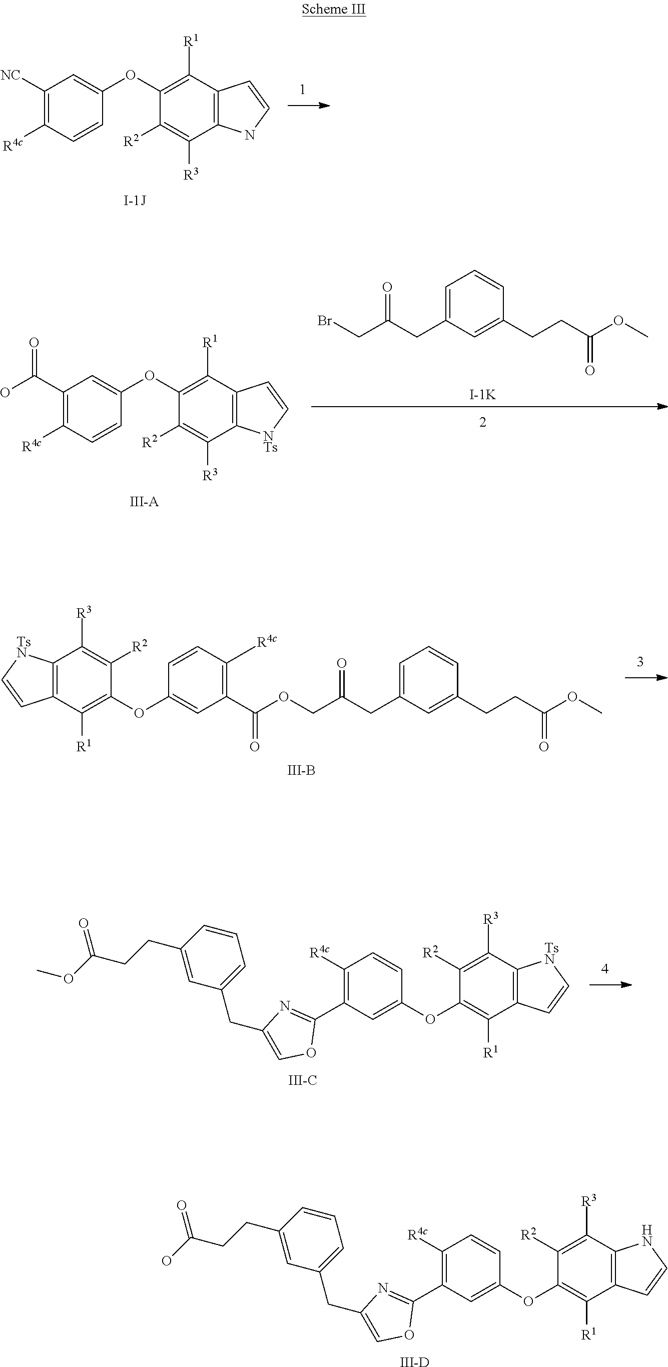

The oxazole compound III-D can be synthesized according to Scheme III. The intermediate I-1J is hydrolyzed (step 1) under the basic conditions into an acid III-A. The acid III-A is condensed with bromide I-1K (step 2) to form an ester III-B. Dehydration (step 3) leads to oxazole III-C. Hydrolysis of III-C yields compound III-D.

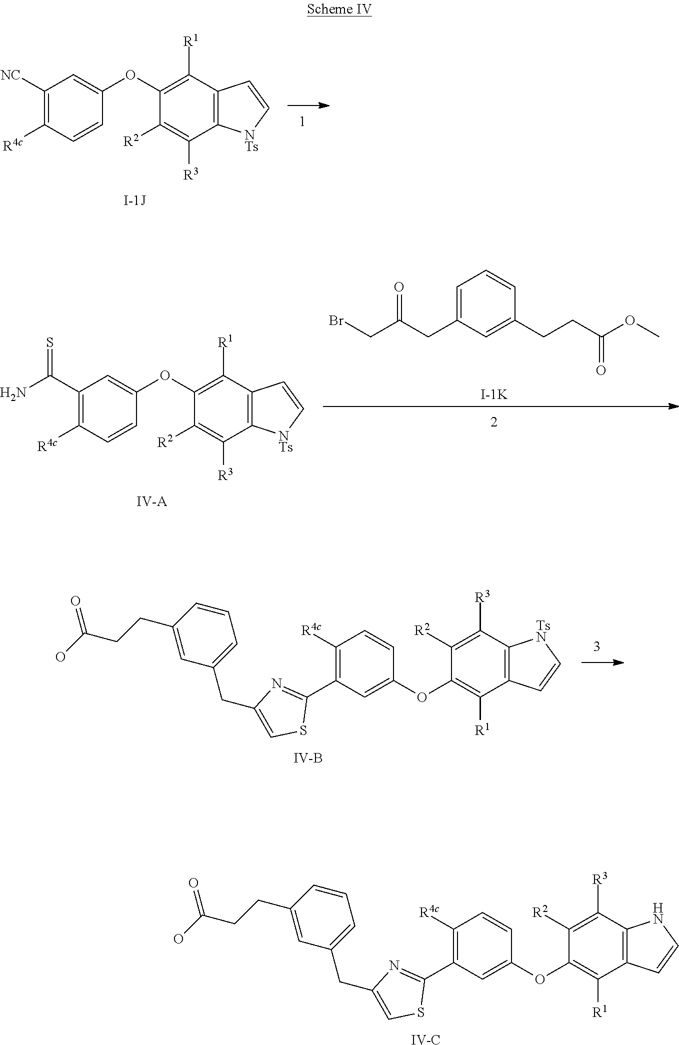

Scheme IV illustrates the preparation of thiazole compound IV-C. The intermediate I-1J is converted (step 1) into thioamide IV-A. Coupling of thioamide IV-A with the intermediate I-1K (step 2) gives IV-B. Removal of the protection groups of IV-B yields compound IV-C.

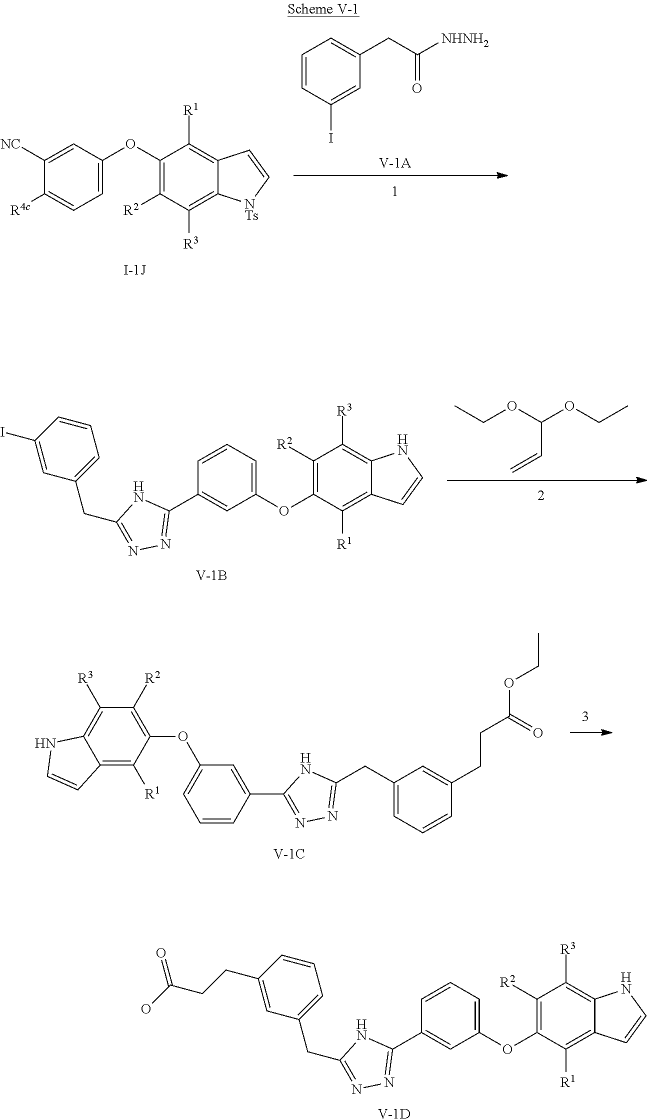

Scheme V-1 illustrates the synthesis of the triazole compound V-1D. The intermediate I-1J is condensed with acylhydrozine V-1A (step 1) to yield triazole V-1B. The intermediate V-1B is condensed with 3,3-diethoxyprop-1-ene (step 2) to give the intermediate V-1C. Hydrolysis of V-1C (step 3) gives compound V-1D.

The chemistry to convert V-1B into V-1C (step 2) is generally applicable to Schemes I to IV if the iodide intermediate similar to V-1B is obtained from the previous cyclization reaction.

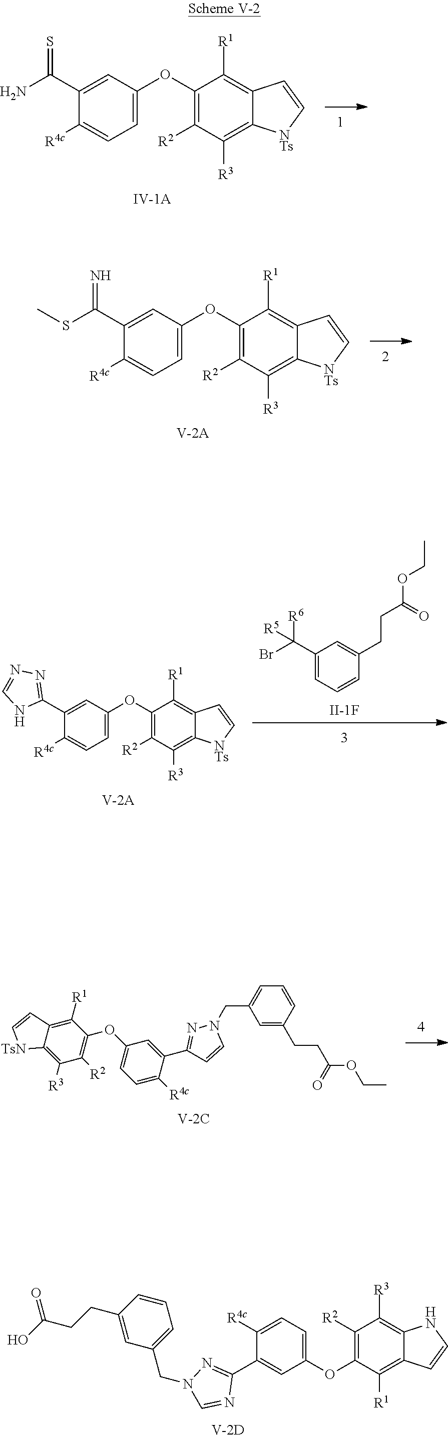

The regio-isomer of triazole compound V-2D can be synthesized as shown in Scheme V-2. Thioamide IV-1A is converted (step 1) into V-2A under methylation conditions. V-2A is cyclized under high temperature with formylhydrozine (step 2) to yield triazole V-2B. The triazole V-2B is alkylated with the intermediate II-1F (step 3) to obtain V-2C. Removal the protection groups of V-2C produces compound V-2D.

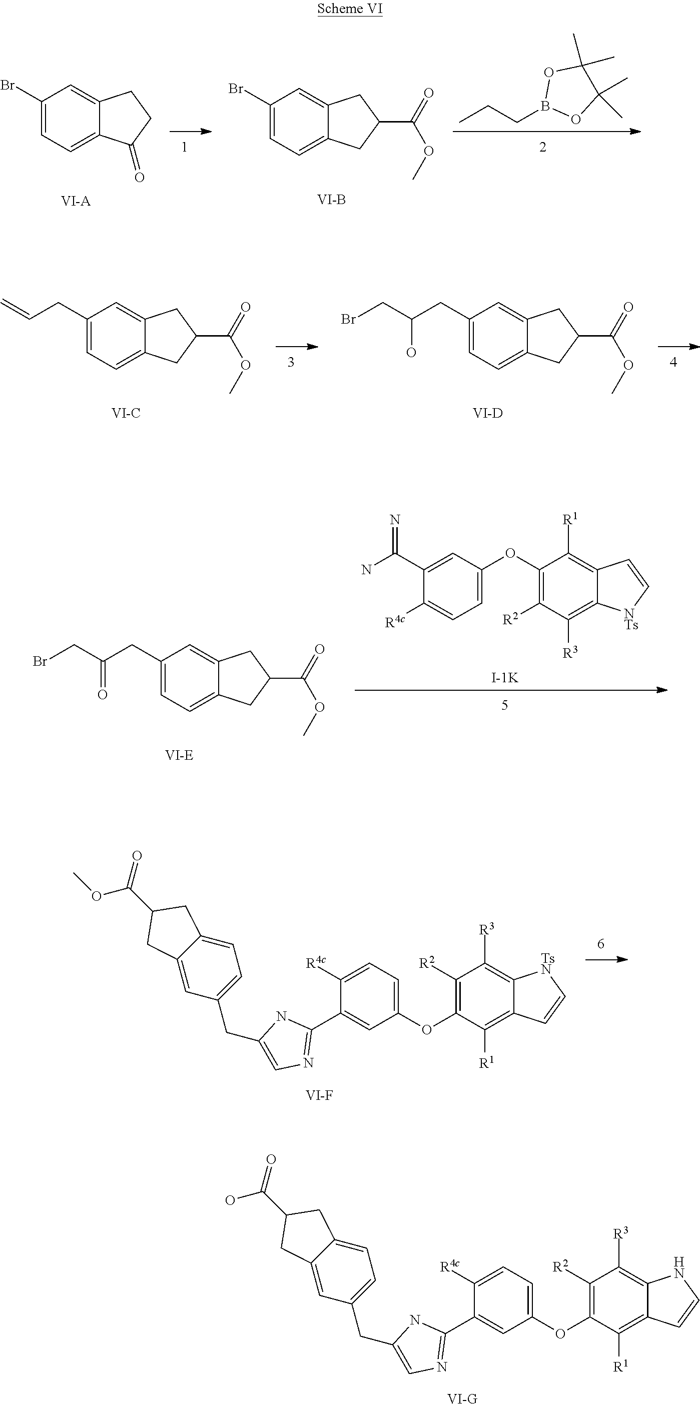

Scheme VI illustrates the preparation of compound VI-G where the cyclic side-chain is substituted. The ketone starting material VI-A is carbonylated (step 1) to an ester VI-B. The intermediate VI-B is coupled with a boronic acid (step 2) to give VI-C. After hydroxybromination (step 3), a bromo-alcohol VI-D is obtained. Oxidation (step 4) affords VI-E. Condensation of VI-E with the intermediate I-1K (step 5) yields VI-F. Hydrolysis of VI-F (step 6) gives compound VI-G.

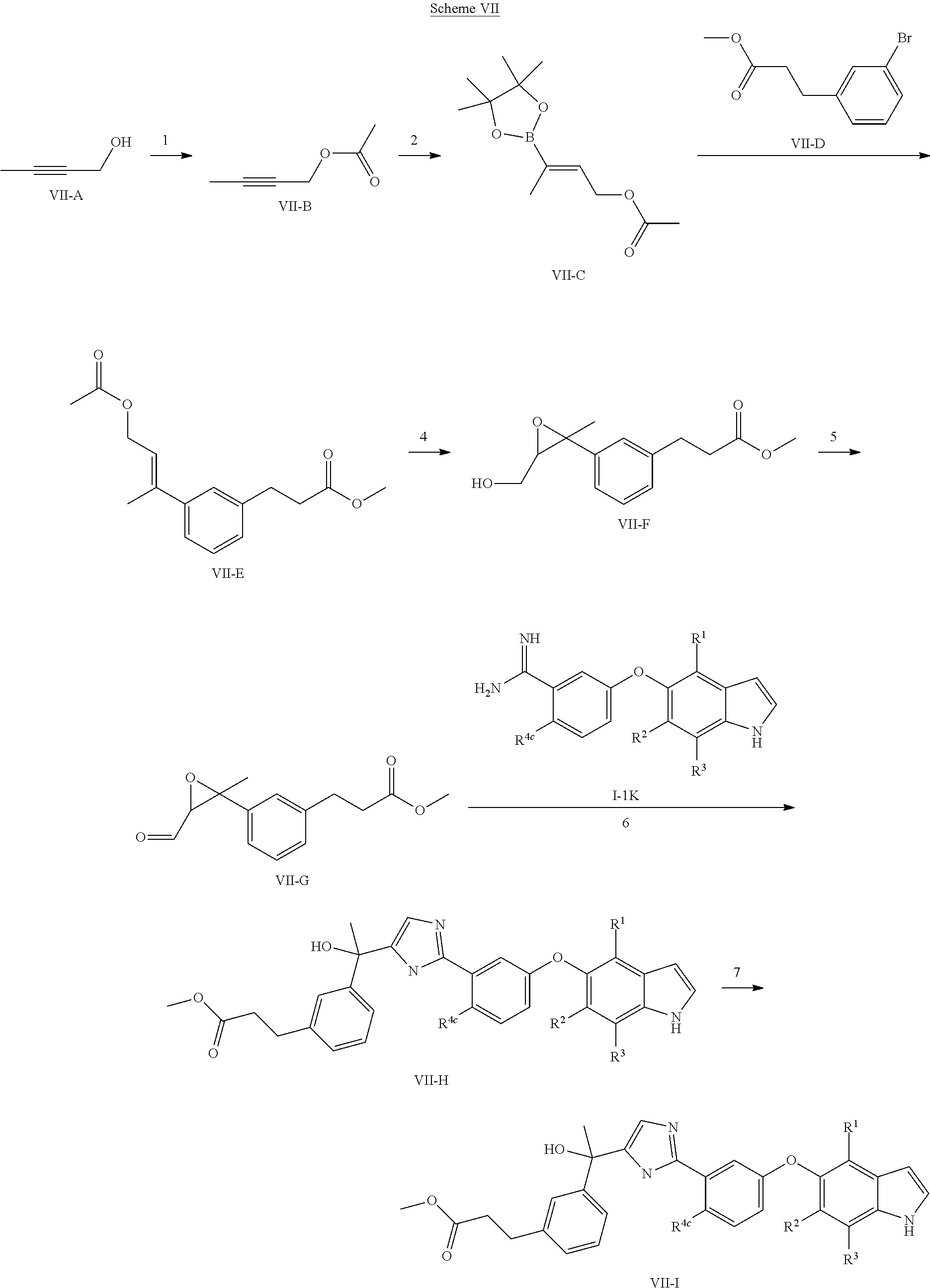

Scheme VII illustrates the imidazole ring formation via an epoxide ring opening approach. The alcohol VII-A is protected (step 1) into an ester VII-B. Borane addition to a triple bond (step 2) generates a boronic ester intermediate VII-C. The boronic ester VII-C is condensed with bromide VII-D (step 3) to give VII-E. Hydrolysis and epoxidation of VII-E produces epoxide VII-F. If chiral catalysis is used here, stereoselective epoxidation may be realized. Oxidation (step 5) of the alcohol VII-F yields an aldehyde epoxide VII-G. Imidazole ring formation is achieved via epoxide ring opening and condensation of VII-G (step 6) to yield VII-H. Hydrolysis of VII-H yields compound VII-I.

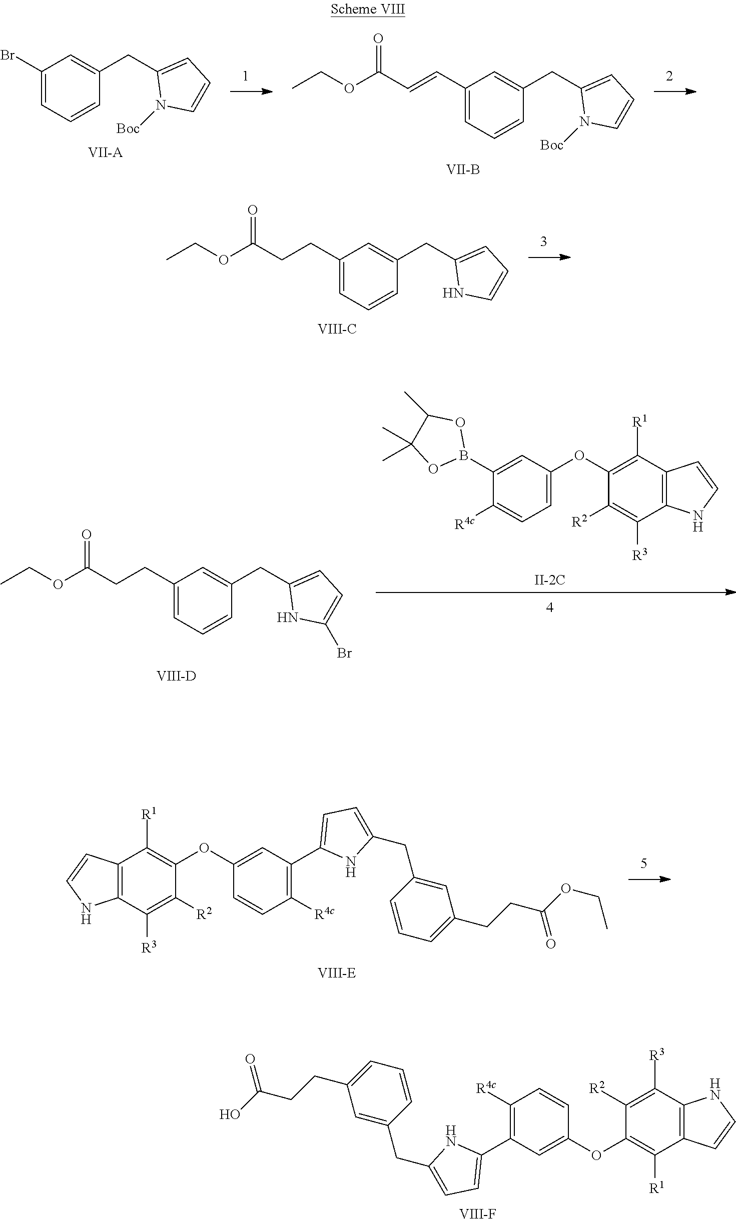

Scheme VIII illustrates the synthesis of the pyrrole compound VIII-F. In step 1, the protected pyrrole analog VIII-A can be coupled with ethyl acrylate by using a catalyst such as Pd(OAc)₂ with heat to obtain VIII-B. The double bond of intermediate VIII-B is reduced, typically under hydrogenation conditions to obtain VIII-C. Bromination of VIII-C gives the bromide VIII-D. Condensation of VIII-D with II-2C using such as Pd(dppf)Cl₂ as catalyst with heat yields VIII-E. Hydrolysis of VIII-E produces compound VIII-F.

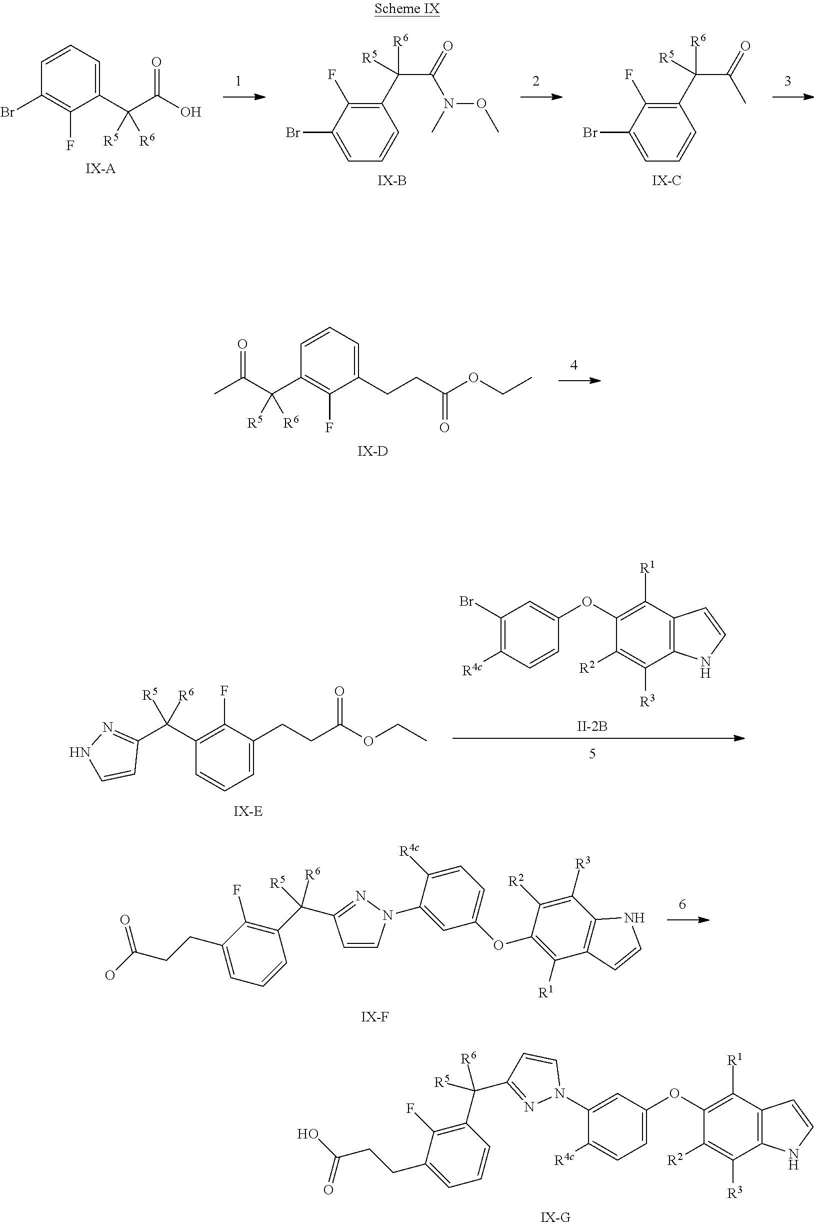

Scheme IX illustrates the synthesis of the compound IX-G. Substituted acid IX-A is converted (step 1) into an amide IX-B, which is further converted (step 2) into a ketone IX-C. The side-chain is installed via a coupling reaction (step 3) to give intermediate IX-D. The intermediate is condensed with DMF-DMA, followed by cyclization (step 4) to yield intermediate IX-E. The coupling of IX-E with II-2B gives the ester IX-F. Hydrolysis affords compound IX-G.

### Analytical Procedures

¹H NMR spectra were recorded with Bruker AC 400 MHz apparatus. Chemical shift (6) are quoted in parts per million (ppm) and coupling constants (J) in hertz (Hz).

LC-MS spectra were obtained with UPLC Acquity device of Waters for liquid chromatography part, coupling with mass spectrometer ZMD of Waters. This system was piloted by MassLynx v4.1 software. Detection was made in UV at 220 nm.

Operational conditions for liquid chromatography part are the following:
Column: Assentis Express C₁₈ 50×2.1 mm, 2.7μ supelco
Eluent: Way A: H₂O+0.02% TFA;
   Way B: CH₃CN+0.014% TFA;
Gradient: To min: 2% B, T₁ min: 98% B, T_{1.3} min: 98% B, T_{1.33} min: 2% B, T_{1.5} min: following injection;
Flow: 1 mL/min;
Temperature: 55° C.
SOD: ESI+30V
UV: 220 nm
Injection: 0.2 μl.

### Preparation of Intermediates

### Intermediate 1

### 5-((4,6-Difluoro-1H-benzo[d]imidazol-5-yl)oxy)-2-fluorobenzonitrile

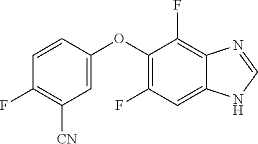 [SMILES: N#Cc1cc(Oc2c(F)cc3[nH]cnc3c2F)ccc1F]

To a solution of 5-(3-amino-2,6-difluoro-4-nitrophenoxy)-2-fluorobenzonitrile (Intermediate 1B, 2.4 g, 7.8 mmol), ammonium chloride (4.2 g, 7.8 mmol) in formic acid (20 mL) and isopropanol (20 mL) was added iron powder (4.30 g, 77.6 mmol). The mixture was stirred at room temperature for four hours, diluted with water (100 mL) and extracted with ethyl acetate (100 mL×2). The combined extracts were washed with water (100 mL) and brine (80 mL). The solution was then dried over sodium sulfate and concentrated. The residue was purified by column chromatography on silica gel to give the title compound as yellow solid (1.8 g, 80%). MS: 290 m/z [M+H]⁺.

### Intermediate 1A

### 2-Fluoro-5-(2,3,6-trifluoro-4-nitrophenoxy)benzonitrile

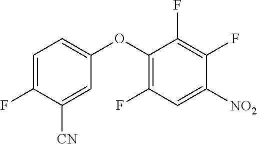 [SMILES: N#Cc1cc(Oc2c(F)cc([N+](=O)[O-])c(F)c2F)ccc1F]

To a solution of 1,2,3,4-tetrafluoro-5-nitrobenzene (2 g, 10.2 mmol) and 2-fluoro-5-hydroxybenzonitrile (1.4 g, 10.2 mmol) in DMF (20 mL) was added potassium carbonate (3 g, 21.6 mmol) and stirred at room temperature for one hour. The mixture was diluted with water (40 mL) and extracted with ethyl acetate (40 mL×2). The combined extracts were washed with water (40 mL) and brine (20 mL), filtered and concentrated. The residue was purified by column chromatography on silica gel to give the title compound as a white solid (2.2 g, 70%). ¹H NMR (400 MHz, CDCl₃) δ 7.83-7.92 (m, 1H), 7.26-7.33 (m, 2H), 7.24 (d, J=3.6 Hz, 1H) ppm. MS: 313 m/z [M+H]⁺.

### Intermediate 1B

### 5-(3-Amino-2,6-difluoro-4-nitrophenoxy)-2-fluorobenzonitrile

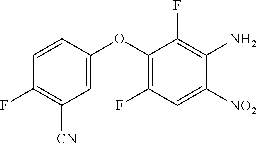 [SMILES: N#Cc1cc(Oc2c(F)cc([N+](=O)[O-])c(N)c2F)ccc1F]

To a stirred solution of 2-fluoro-5-(2,3,6-trifluoro-4-nitrophenoxy)benzonitrile (3.00 g, 9.61 mmol) and triethylamine (4.1 mL, 29 mmol) in DMF (30 mL) was added ammonium carbonate (1.10 g, 11.4 mmol). After four hours at room temperature, the mixture was diluted with water (1×100 mL) and extracted with ethyl acetate (2×90 mL). The combined extracts were washed with water (1×100 mL) and brine (1×80 mL), dried over sodium sulfate and concentrated. Crude product was obtained as a yellow solid (2.40 g, 81%). MS: 310 m/z [M+H]⁺.

### Intermediate 2

### 5-((4-Bromo-6,7-difluoro-1H-indol-5-yl)oxy)-2-fluorobenzonitrile

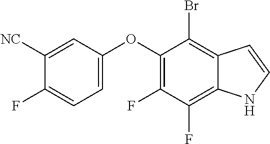 [SMILES: N#Cc1cc(Oc2c(F)c(F)c3[nH]ccc3c2Br)ccc1F]

To a solution of 5-(4-amino-2-bromo-5,6-difluoro-3-((trimethylsilyl)ethynyl)phenoxy)-2-fluorobenzonitrile (Intermediate 2E, 700 mg, 1.6 mmol) in DMF (7 mL) was added CuI (608 mg, 3.2 mmol) and stirred at 100° C. in glove box for four hours. The reaction mixture was diluted with water and extracted with ethyl acetate (50 mL×3). The organic phase was washed with water, brine, dried with sodium sulfate, filtered and concentrated. The residue was purified by flash chromatography over silica to give the title compound as a yellow solid (362 mg, 62%). ¹H NMR (400 MHz, CDCl₃) δ 8.63 (s, 1H), 7.37-7.39 (m, 1H), 7.14-7.23 (m, 2H), 7.04-7.06 (m, 1H), 6.67-6.69 (m, 1H) ppm.

### Intermediate 2A

### 1-Bromo-2,3,4-trifluoro-5-nitrobenzene

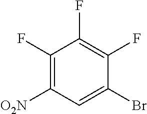 [SMILES: O=[N+]([O-])c1cc(Br)c(F)c(F)c1F]

To a solution of 1,2,3-trifluoro-4-nitrobenzene (30 g, 169.5 mmol) in concentrated sulfuric acid (150 mL) was added 1,3-dibromo-5,5-dimethylimidazolidine-2,4-dione (24 g, 84.7 mmol) at 0° C. and stirred at room temperature overnight. The mixture was slowly and carefully added to ice water (600 g ice and 100 mL water) to keep the temperature below 30° C. and extracted with heptane (300 mL×3). The combined organic extracts were washed with water and brine, dried over magnesium sulfate, filtered and evaporated to dryness. The resulting residue was purified by flash chromatography over silica (heptane) to give the title compound (32 g, 75%) as a yellow oil. ¹H NMR (400 MHz, CDCl₃) δ 8.21 (td, J=7.2, 2.8 Hz, 1H) ppm.

### Intermediate 2B

### 5-(6-Bromo-2,3-difluoro-4-nitrophenoxy)-2-fluorobenzonitrile

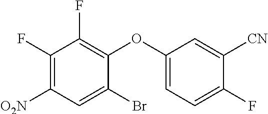 [SMILES: N#Cc1cc(Oc2c(Br)cc([N+](=O)[O-])c(F)c2F)ccc1F]

To a solution of 1-bromo-2,3,4-trifluoro-5-nitrobenzene (Intermediate 2A, 32 g, 125.5 mmol) in DMF (250 mL) were added 2-fluoro-5-hydroxybenzonitrile (18.9 g, 138.0 mmol) and potassium carbonate (26 g, 1.5 mmol) at room temperature and stirred for one hour. The reaction mixture was diluted with water (300 mL) and extracted with ethyl acetate (250 mL×3). The combined organic extracts were washed with water and brine, dried over magnesium sulfate, filtered and concentrated. The residue was purified by flash chromatography over silica to give the title compound as a yellow solid (15 g, 30%). MS: 373, 375 m/z [M+H]⁺. ¹H NMR (400 MHz, CDCl₃) δ 8.30 (dd, J=7.2, 2.4 Hz, 1H), 7.20-7.25 (m, 2H), 7.15-7.17 (m, 1H) ppm.

### Intermediate 2C

### 5-(4-Amino-6-bromo-2,3-difluorophenoxy)-2-fluorobenzonitrile

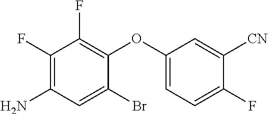 [SMILES: N#Cc1cc(Oc2c(Br)cc(N)c(F)c2F)ccc1F]

To a solution of 5-(6-bromo-2,3-difluoro-4-nitrophenoxy)-2-fluorobenzonitrile (Intermediate 2B, 5 g, 13.4 mmol) in ethanol (100 mL) and water (30 mL) were added iron powder (3 g, 53.6 mmol) and ammonium chloride (5.8 g, 107.5 mmol). The reaction mixture was stirred at 80° C. for four hours, cooled to room temperature, diluted with water (100 mL) and extracted with ethyl acetate (100 mL×3). The combined organic extracts were washed with water, brine, dried over magnesium sulfate, filtered and evaporated to dryness. The residue was purified by flash chromatography over silica (heptane/ethyl acetate, v/v, 10/1) to give the title compound a yellow solid (3 g, 65%). MS: 343, 345 m/z [M+H]⁺.

### Intermediate 2D

### 5-(4-Amino-2-bromo-5,6-difluoro-3-iodophenoxy)-2-fluorobenzonitrile

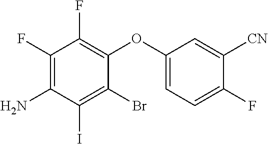 [SMILES: N#Cc1cc(Oc2c(F)c(F)c(N)c(I)c2Br)ccc1F]

To a solution of 5-(4-amino-6-bromo-2,3-difluorophenoxy)-2-fluorobenzonitrile (Intermediate 2C, 6.7 g, 19.6 mmol) in acetic acid (200 mL) was added NIS (4.4 g, 19.6 mmol). The mixture was stirred at room temperature for three hours, diluted with water (200 mL) and extracted with ethyl acetate (150 mL×3). The combined organic extracts were washed with water, brine, dried over magnesium sulfate, filtered and evaporated to dryness. The residue was purified by flash chromatography over silica (petroleum ether/dichloromethane, v/v, 2/1) to give the title compound as a yellow solid (8.2 g, 89%). ¹H NMR (400 MHz, CDCl₃) δ 7.16-7.18 (m, 2H), 7.03-7.04 (m, 1H), 4.56 (s, 2H) ppm. MS: 469, 471 m/z [M+H]⁺.

### Intermediate 2E

### 5-(4-Amino-2-bromo-5,6-difluoro-3-((trimethylsilyl)ethynyl)phenoxy)-2-fluorobenzonitrile

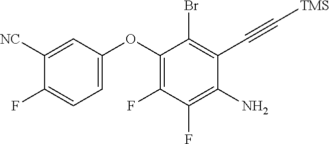 [SMILES: C[Si](C)(C)C#Cc1c(N)c(F)c(F)c(Oc2ccc(F)c(C#N)c2)c1Br]

To a solution of 5-(4-amino-2-bromo-5,6-difluoro-3-iodophenoxy)-2-fluorobenzonitrile (Intermediate 2D, 8.1 g, 17.3 mmol) in DMF (200 mL) were added trimethylsilylacetylene (3.4 g, 34.6 mmol), Pd(Ph₃P)₂Cl₂ (1.2 g, 1.7 mmol), CuI (323 mg, 1.7 mmol) and triethylamine (3.5 g, 34.6 mmol). The reaction mixture was stirred at 30° C. under nitrogen for three hours, diluted with water (300 mL) and extracted with ethyl acetate (250 mL×3). The combined organic extracts were washed with water, brine, dried with sodium sulfate, filtered and concentrated. The residue was purified by flash chromatography over silica (petroleum ether/dichloromethane, v/v, 8/1) to give the title compound as a yellow solid (5 g, 64%). MS: 439, 441 m/z [M+H]⁺.

The following intermediate was prepared utilizing the procedures described for Intermediate 2.

| | | | LCMS |
|---|---|---|---|
| No. | Structure | Name | (m/z) |
| 2-1 | 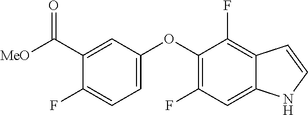 [SMILES: COC(=O)c1cc(Oc2c(F)cc3[nH]ccc3c2F)ccc1F] | methyl 5-((4,6- difluoro-1H-indol-5- yl)oxy)-2- fluorobenzoate | 322 [M + H]⁺ |

### Intermediate 3

### 2-Fluoro-5-((4,6,7-trifluoro-1H-indol-5-yl)oxy)benzonitrile

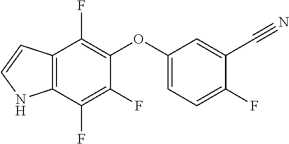 [SMILES: N#Cc1cc(Oc2c(F)c(F)c3[nH]ccc3c2F)ccc1F]

To a stirred solution of 2-fluoro-5-(2,3,6-trifluoro-4-nitrophenoxy)benzonitrile (Intermediate 1A, 5 g, 16 mmol) in THF (50 mL) was added vinylmagnesium bromide (100 mL, 100 mmol, 1M in THF). The reaction mixture was stirred at −78° C. for one hour, quenched with water and diluted with ethyl acetate (200 mL). The organic phase was washed with water (100 mL×3) and brine (50 mL×2), dried with sodium sulfate, filtered and concentrated to give a residue, which was purified by flash chromatography over silica (petroleum ether/ethyl acetate, v/v, 5/1) to afford the title compound as white solid (700 mg, 14%). ¹H NMR (400 MHz, CDCl₃) δ 8.58 (s, 1H), 7.29 (dd, J=6.0, 3.2 Hz, 1H), 7.25 (s, 1H), 7.18 (d, J=8.4 Hz, 1H), 7.13 (dd, J=8.4, 4.0 Hz, 1H), 6.71 (dd, J=6.0, 3.2 Hz, 1H) ppm. MS: 307 m/z [M+H]⁺.

### Intermediate 4

### 5-((6,7-Difluoro-4-methyl-1H-indol-5-yl)oxy)-2-fluorobenzonitrile

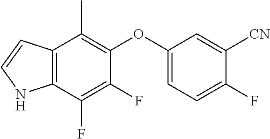 [SMILES: Cc1c(Oc2ccc(F)c(C#N)c2)c(F)c(F)c2[nH]ccc12]

A mixture of 5-(4-amino-2,3-difluoro-6-methyl-5-((trimethylsilyl)ethynyl)phenoxy)-2-fluorobenzonitrile (Intermediate 4D, 1.4 g, 3.74 mmol) and copper (I) Iodide (1.4 g, 7.48 mmol) in DMF (15 mL) was heated at 100° C. in a glove box overnight. The insoluble material was removed by filtration and the filtrate was diluted with ethyl acetate (50 mL). The organic phase was washed with brine (20 mL×3), dried over sodium sulfate, filtered and concentrated to give a residue, which was purified by flash chromatography over silica (petroleum ether/ethyl acetate, v/v, 4/1) to afford the title compound as a yellow solid (800 mg, 71%). ¹H-NMR (400 MHz, CDCl₃) δ 8.68 (s, 1H), 7.33-7.32 (m, 1H), 7.22-7.13 (m, 2H), 7.03-7.01 (m, 1H), 6.63-6.61 (m, 1H), 2.35 (s, 3H) ppm.

### Intermediate 4A

### 5-(2,3-Difluoro-6-methyl-4-nitrophenoxy)-2-fluorobenzonitrile

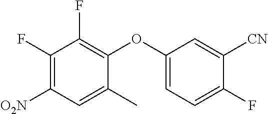 [SMILES: Cc1cc([N+](=O)[O-])c(F)c(F)c1Oc1ccc(F)c(C#N)c1]

To a solution of 5-(6-bromo-2,3-difluoro-4-nitrophenoxy)-2-fluorobenzonitrile (Intermediate 2B 7.1 g, 19 mmol) in dioxane (60 mL) and water (20 mL) was added sodium bicarbonate (4.03 g, 38 mmol), Pd(dppf)Cl₂ (1.39 g, 1.9 mmol) and methylboronic acid (5.7 g, 95.0 mmol) under nitrogen atmosphere. The reaction mixture was stirred at 80° C. for four hours and the insoluble materials were removed by filtration. The filtrate was diluted with ethyl acetate (150 mL), washed with water (40 mL×2), brine (40 mL), dried over sodium sulfate, filtered and concentrated. The residue was purified by flash chromatography over silica (petroleum ether/ethyl acetate, v/v, 20/1) to afford the title compound as a yellow solid (1.9 g, 33%). ¹H-NMR (400 MHz, CDCl₃) δ 7.90 (dd, J=1.6, 1.6 Hz, 1H), 7.27-7.18 (m, 2H), 7.14-7.12 (m, 1H), 2.33 (s, 3H) ppm.

### Intermediate 4B

### 5-(4-Amino-2,3-difluoro-6-methylphenoxy)-2-fluorobenzonitrile

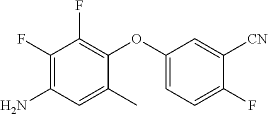 [SMILES: Cc1cc(N)c(F)c(F)c1Oc1ccc(F)c(C#N)c1]

To a solution of 5-(2,3-difluoro-6-methyl-4-nitrophenoxy)-2-fluorobenzonitrile (Intermediate 4A, 1.9 g, 6.16 mmol) in ethanol (30 mL) and water (10 mL) was added ammonium chloride (2.66 g, 49.3 mmol) and Iron powder (1.38 g, 24.6 mmol). The reaction mixture was stirred at 70° C. for four hours. The insoluble materials were removed by suction. The filtrate was diluted with ethyl acetate (100 mL), washed with water (30 mL×2), brine (30 mL), dried over sodium sulfate, filtered and concentrated to give the title compound as yellow solid (1.54 g, 90%). MS: 279 m/z [M+H]⁺.

### Intermediate 4C

### 5-(4-Amino-2,3-difluoro-5-iodo-6-methylphenoxy)-2-fluorobenzonitrile

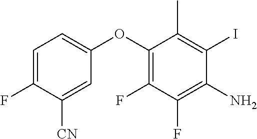 [SMILES: Cc1c(I)c(N)c(F)c(F)c1Oc1ccc(F)c(C#N)c1]

A mixture of 5-(4-amino-2,3-difluoro-6-methylphenoxy)-2-fluorobenzonitrile (Intermediate 4B, 1.54 g, 5.54 mmol) and NIS (1.24 g, 5.54 mmol) in acetic acid (20 mL) was stirred at 30° C. for one hour. The reaction was quenched with water (20 mL) and extracted with ethyl acetate (20 mL×3). The combined organic extracts were washed with water (20 mL×3), brine (20 mL), dried over sodium sulfate, filtered and concentrated. The residue was purified by flash chromatography over silica (petroleum ether/ethyl acetate, v/v, 20/1) to afford the title compound as a yellow solid (2 g, 90%). MS: 405 m/z [M+H]⁺.

### Intermediate 4D

### 5-(4-Amino-2,3-difluoro-6-methyl-5-((trimethylsilyl)ethynyl)phenoxy)-2-fluorobenzonitrile

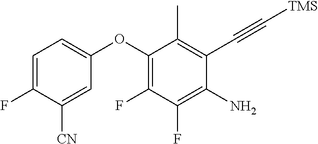 [SMILES: Cc1c(C#C[Si](C)(C)C)c(N)c(F)c(F)c1Oc1ccc(F)c(C#N)c1]

To a solution of 5-(4-amino-2,3-difluoro-5-iodo-6-methylphenoxy)-2-fluorobenzonitrile (Intermediate 4C, 2 g, 4.96 mmol) in DMF (20 mL) were added Pd(dppf)Cl₂ (366 mg, 0.5 mmol), CuI (95 mg, 0.50 mmol), triethylamine (1.4 mL, 9.92 mmol) and ethynyltrimethylsilane (729 mg, 7.44 mmol). The reaction mixture was stirred at 30° C. for three hours under the nitrogen atmosphere. The reaction was quenched with water (30 mL) and extracted with ethyl acetate (20 mL×3). The combined organic phase was washed with water and brine (10 mL), dried over sodium sulfate, filtered and concentrated. The residue was purified by flash chromatography over silica (petroleum ether/ethyl acetate, v/v, 4/1) to afford the title compound as a yellow solid (1.4 g, 76%). MS: 375 m/z [M+H]⁺.

### Intermediate 5

### 2-Fluoro-5-((4-(trifluoromethyl)-1H-indol-5-yl)oxy)benzonitrile

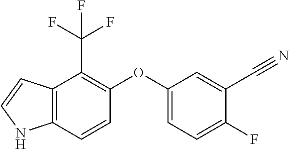 [SMILES: N#Cc1cc(Oc2ccc3[nH]ccc3c2C(F)(F)F)ccc1F]

To a solution of 5-(4-amino-3-ethynyl-2-(trifluoromethyl)phenoxy)-2-fluorobenzonitrile (Intermediate 5D, 338 mg, 1.06 mmol) in DMF (4 mL) was added CuI (301 mg, 1.58 mmol) and stirred at 100° C. for 30 hours under nitrogen atmosphere. The reaction mixture was filtered, concentrated and the residue was purified by flash chromatography over silica (petroleum ether/ethyl acetate, v/v, 2/1) to give the title compound as a solid (498 mg, 79%). ¹H NMR (400 MHz, CDCl₃) δ 8.54 (br s, 1H), 7.59-7.62 (d, 1H), 7.43-7.45 (m, 1H), 7.14-7.25 (m, 2H), 7.06-7.08 (m, 1H), 6.85-6.91 (m, 2H) ppm. MS: 321 m/z [M+H]⁺.

### Intermediate 5A

### 1,3-Difluoro-4-nitro-2-(trifluoromethyl)benzene

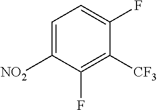 [SMILES: O=[N+]([O-])c1ccc(F)c(C(F)(F)F)c1F]

To a stirred mixture of concentrated nitric acid (35 g) and concentrated sulfuric acid (70 g) was added 1,3-difluoro-2-(trifluoromethyl)benzene (23 g, 126 mmol) drop wise at 50° C. The reaction mixture was stirred at 50° C. for one hour, cooled to room temperature and poured onto 500 g of ice. The mixture was extracted with dichloromethane. The combined organic extracts were dried over sodium sulfate, filtered and concentrated. The resulting residue was purified by flash chromatography over silica (0-20% ethyl acetate in petroleum ether) to give the title compound as an oil (23 g, 79%). ¹H NMR (400 MHz, DMSO-d₆) δ 8.56-8.62 (m, 1H), 7.62-7.66 (m, 1H) ppm.

### Intermediate 5B

### ((3-Fluoro-6-nitro-2-(trifluoromethyl)phenyl)ethynyl)trimethylsilane

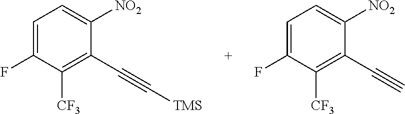 [SMILES: C[Si](C)(C)C#Cc1c([N+](=O)[O-])ccc(F)c1C(F)(F)F ; C#Cc1c([N+](=O)[O-])ccc(F)c1C(F)(F)F]

To a solution of 1,3-difluoro-4-nitro-2-(trifluoromethyl)benzene (Intermediate 5A, 7.9 g, 3.47 mmol) in THF (100 mL) were added ethynyltrimethylsilane (4.09 g, 4.17 mmol) and sodium bis(trimethylsilyl)amide (20.8 mL, 4.17 mmol) dropwise at 0° C. under nitrogen atmosphere. The reaction mixture was stirred at 0° C. for 30 minutes, quenched with water (250 mL) and extracted with ethyl acetate (100 mL×3). The combined organic extracts were dried with sodium sulfate and concentrated. The resulting residue was purified by flash chromatography over silica (petroleum ether/ethyl acetate, v/v, 4/1) to afford ((3-fluoro-6-nitro-2-(trifluoromethyl)phenyl)ethynyl)trimethylsilane as a yellow oil (1.46 g, 14%). ¹H NMR (400 MHz, CDCl₃) δ 8.04-8.07 (m, 1H), 7.25-7.30 (m, 1H), 0.29 (s, 9H) ppm. 2-Ethynyl-4-fluoro-1-nitro-3-(trifluoromethyl)benzene (2.4 g, 29%) was also obtained as a yellow solid. ¹H NMR (400 MHz, CDCl₃) δ 8.07-8.10 (m, 1H), 7.28-7.38 (m, 1H), 3.88 (s, 1H) ppm.

### Intermediate 5C

### 5-(3-Ethynyl-4-nitro-2-(trifluoromethyl)phenoxy)-2-fluorobenzonitrile

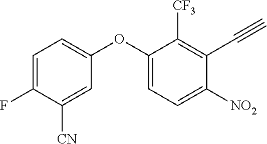 [SMILES: C#Cc1c([N+](=O)[O-])ccc(Oc2ccc(F)c(C#N)c2)c1C(F)(F)F]

To a solution of ((3-fluoro-6-nitro-2-(trifluoromethyl)phenyl)ethynyl)trimethylsilane (Intermediate 5B, 0.5 g, 1.63 mmol) in DMF (10 mL) were added 2-fluoro-5-hydroxybenzonitrile (336 mg, 2.45 mmol) and potassium carbonate (336 mg, 2.45 mmol) and stirred at room temperature for one hour. The reaction mixture was quenched with water (30 mL) and extracted with ethyl acetate (10 mL×3). The combined organic phase was dried with sodium sulfate, filtered and concentrated. The resulting residue was purified by flash chromatography over silica (0-30% ethyl acetate in petroleum ether) to afford the title compound as a yellow solid (849 mg, 67%). MS: 351 m/z [M+H]⁺.

### Intermediate 5D

### 5-(4-Amino-3-ethynyl-2-(trifluoromethyl)phenoxy)-2-fluorobenzonitrile

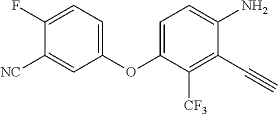 [SMILES: C#Cc1c(N)ccc(Oc2ccc(F)c(C#N)c2)c1C(F)(F)F]

To a solution of 5-(3-ethynyl-4-nitro-2-(trifluoromethyl)phenoxy)-2-fluorobenzonitrile (Intermediate 5C, 849 mg, 2.42 mmol) in a 1:1 mixture of methanol and water (16 mL) was added iron powder (679 mg, 12.1 mmol) and ammonium chloride (19.4 mg, 19.4 mmol). The reaction mixture was stirred at 60° C. for two hours, cooled to room temperature, filtered through a pad of celite. The filtrate cake was washed with dichloromethane (5×30 mL). The combined dichloromethane washing and filtrate was washed with brine (30 mL), dried over sodium sulfate, filtered and concentrated. The residue was purified by silica gel chromatography to afford the title compound as an oil (630 mg, 81%). ¹H NMR (400 MHz, CDCl₃) δ 7.15-7.17 (m, 2H), 7.03-7.06 (m, 1H), 6.87-6.95 (m, 2H), 4.59 (br s, 2H), 3.78 (s, 1H) ppm. MS: 321 m/z [M+H]⁺.

### Intermediate 6

### 5-((4-Bromo-6-fluoro-1H-indol-5-yl)oxy)-2-fluorobenzonitrile

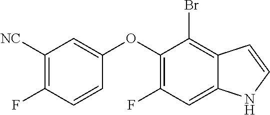 [SMILES: N#Cc1cc(Oc2c(F)cc3[nH]ccc3c2Br)ccc1F]

To a solution of 5-(2-bromo-6-fluoro-3-methyl-4-nitrophenoxy)-2-fluorobenzonitrile (Intermediate 6B, 124 g, 336 mmol) in DMF (1 L) was added DMF-DMA (160 g, 1.34 mol, 178 mL) and the solution was stirred at 100° C. for fix hours. Six batches of this reaction (total of 744 g of 5-(2-bromo-6-fluoro-3-methyl-4-nitrophenoxy)-2-fluorobenzonitrile used) were combined, cooled to room temperature, poured into ice-water (20 L) and extracted with ethyl acetate (8 L×2). The combined organic extracts were washed with water (10 L), brine (10 L), dried over sodium sulfate, filtered and concentrated to afford (E)-5-(2-bromo-3-(2-(dimethylamino)vinyl)-6-fluoro-4-nitrophenoxy)-2-fluorobenzonitrile as a black oil (786 g, 92%). ¹H NMR: (400 MHz CDCl₃) δ 7.38 (d, J=8.8 Hz, 1H), 7.19 (s, 1H), 7.10-7.12 (m, 2H), 6.44 (d, J=13.6 Hz, 1H), 4.96 (d, J=13.6 Hz, 1H), 2.80 (s, 6H) ppm.

To the above solution of (E)-5-(2-bromo-3-(2-(dimethylamino)vinyl)-6-fluoro-4-nitrophenoxy)-2-fluorobenzonitrile (100 g, 236 mmol) in acetic acid (800 mL) and toluene (800 mL) was added silica gel (42.5 g, 707 mmol). The reaction was brought to 50° C. and iron powder (132 g, 2.4 mol) was added portion wise. The suspension was stirred at 100° C. for 12 hours, cooled to room temperature, filtered through Celite and rinsed with ethyl acetate (5 L). The filtrate was diluted with water (10 L) and extracted with ethyl acetate (5 L×2). The combined organic extracts were washed with water (10 L) and brine (10 L), dried over sodium sulfate, filtered and concentrate under reduced pressure to give a dark brown oil. The oil was purified by flash chromatography over silica (1-20% of ethyl acetate in petroleum ether). Three batches of this reaction were carried out (total 300 μg of 5-(2-bromo-3-(2-(dimethylamino)vinyl)-6-fluoro-4-nitrophenoxy)-2-fluorobenzonitrile were used) to afford the title compound as a white solid (200 g, 81%). ¹H NMR: (400 MHz CDCl₃) δ 8.41 (s, 1H), 7.25-7.26 (m, 1H), 7.15-7.18 (m, 3H), 6.95-7.09 (m, 1H), 6.55 (t, J=2.8 Hz, 1H) ppm.

### Intermediate 6A

### 3-Bromo-1,2-difluoro-4-methyl-5-nitrobenzene

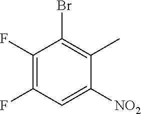

To a solution of 1,2-difluoro-4-methyl-5-nitrobenzene (150 g, 866 mmol) in trifluroacetic acid (800 mL) were added 1,3-dibromo-5,5-dimethylimidazolidine-2,4-dione (136 g, 476 mmol) and concentrated sulfuric acid (200 mL). The solution was stirred at 25° C. for 10 hours. Three batches of reaction suspension (total 450 g of 1,2-difluoro-4-methyl-5-nitrobenzene) were combined and poured into ice water (5 L) and stirred for 15 minutes and extracted with petroleum ether (4 L×2). The combined organic extracts were washed with brine (5 L), dried over sodium sulfate, filtered and concentrated. The resulting oil was purified by flash chromatography over silica (100% petroleum ether) to afford the title compound as a yellow oil (417 g, 64%). ¹H NMR (400 MHz CDCl₃) δ 7.68 (q, J=6.0 Hz, 1H), 2.55 (s, 3H) ppm.

### Intermediate 6B

### 5-(2-Bromo-6-fluoro-3-methyl-4-nitrophenoxy)-2-fluorobenzonitrile

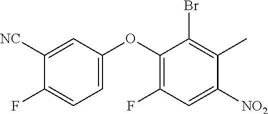 [SMILES: Cc1c([N+](=O)[O-])cc(F)c(Oc2ccc(F)c(C#N)c2)c1Br]

To a solution of 3-bromo-1,2-difluoro-4-methyl-5-nitrobenzene (Intermediate 6A, 207 g, 820 mmol) in DMF (1 L) were added 2-fluoro-5-hydroxybenzonitrile (118 g, 861 mmol) and potassium carbonate (227 g, 1.6 mol). The suspension was stirred at 100° C. for one hour. Two batches of the reaction mixture (from a total 414 g of 3-bromo-1,2-difluoro-4-methyl-5-nitrobenzene) were combined, cooled, poured into ice-water (7 L) and extracted with ethyl acetate (3 L×2). The combined organic extracts were washed with water (5 L) and brine (3 L), dried over sodium sulfate, filtered and concentrated to afford the title compound as a yellow solid (585 g, 97%). The crude product was used without further purification. ¹H NMR: (400 MHz CDCl₃) δ 7.68 (d, J=9.2 Hz, 1H), 7.10-7.14 (m, 2H), 7.01-7.02 (m, 1H), 2.58 (s, 3H).

The following intermediates were prepared utilizing the procedure described for Intermediate

| | | | | MS m/z |
|---|---|---|---|---|
| No. | Structure | Name | ¹H NMR | [M + H]⁺ |
| 6-1 | 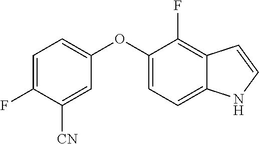 [SMILES: N#Cc1cc(Oc2ccc3[nH]ccc3c2F)ccc1F] | 2-fluoro-5-((4-fluoro- 1H-indol-5- yl)oxy)benzonitrile | | 271 |
| 6-2 | 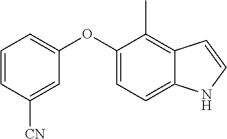 [SMILES: Cc1c(Oc2cccc(C#N)c2)ccc2[nH]ccc12] | 3-((4-methyl-1H-indol- 5-yl)oxy)benzonitrile | (400 MHz, DMSO-d₆) δ 7.52-7.44 (m, 2H), 7.42-7.40 (m, 1H), 7.31 (d, J = 8.4 Hz, 1H), 7.21-7.20 (m, 1H), 7.14-7.11 (m, 1H), 6.81 (d, J = 8.8 Hz, 1H), 6.52-6.51 (m, 1H), 2.26 (s, 3H) ppm. | 249 |
| 6-3 | 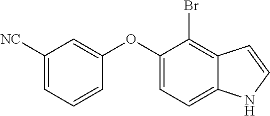 [SMILES: N#Cc1cccc(Oc2ccc3[nH]ccc3c2Br)c1] | 3-((4-bromo-1H-indol-5- yl)oxy)benzonitrile | (400 MHz, CDCl₃) δ 8.50 (s, 1H), 7.40-7.32 (m, 3H), 7.29 (d, J = 7.6 Hz, 1H), 7.17 (dd, J = 8.0, 2.4 Hz, 1H), 7.08-7.06 (m, 1H), 6.95 (d, J = 8.8 Hz, 1H), 6.65- 6.40 (m, 1H) ppm. | 313, 315 |
| 6-4 | 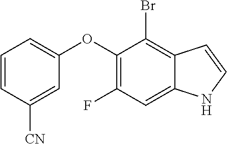 [SMILES: N#Cc1cccc(Oc2c(F)cc3[nH]ccc3c2Br)c1] | 3-((4-bromo-6-fluoro- 1H-indol-5- yl)oxy)benzonitrile | | 331, 333 |
| 6-5 | 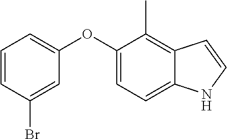 [SMILES: Cc1c(Oc2cccc(Br)c2)ccc2[nH]ccc12] | 5-(3-bromophenoxy)-4- methyl-1H-indole | (400 MHz, DMSO-d₆) δ 7.41-7.39 (m, 1H), 7.30 (d, J = 8.4 Hz, 1H), 7.27-7.23 (m, 1H), 7.18-7.16 (m, 1H), 6.91-6.90 (m, 1H), 6.84-6.79 (m, 2H), 6.51-6.50 (m, 1H), 2.26 (s, 3H) ppm. | 302, 304 |
| 6-6 | 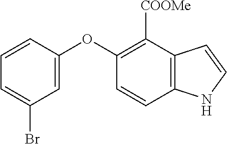 [SMILES: COC(=O)c1c(Oc2cccc(Br)c2)ccc2[nH]ccc12] | methyl 5-(3- bromophenoxy)-1H- indole-4-carboxylate | | 346, 348 |
| 6-7 | 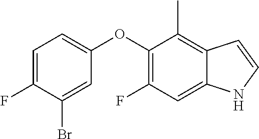 [SMILES: Cc1c(Oc2ccc(F)c(Br)c2)c(F)cc2[nH]ccc12] | 5-(3-bromo-4- fluorophenoxy)-6-fluoro- 4-methyl-1H-indole | (400 MHz, CDCl₃) δ 8.29 (s, 1H), 7.30-7.23 (m, 1H), 7.08 (d, J = 10.0 Hz, 1H), 7.04-6.99 (m, 2H), 6.84-6.77 (m, 1H), 6.57 (s, 1H), 2.39 (s, 3H) ppm. | |
| 6-8 | 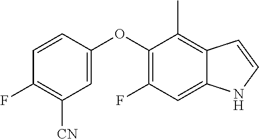 [SMILES: Cc1c(Oc2ccc(F)c(C#N)c2)c(F)cc2[nH]ccc12] | 2-fluoro-5-((6-fluoro-4- methyl-1H-indol-5- yl)oxy)benzonitrile | (400 MHz, CDCl₃) δ 8.28 (s, 1H), 7.27 (s, 1H), 7.20-7.07 (m, 3H), 6.98 (dd, J = 4. 8, 2.8 Hz, 1H), 6.58 (s, 1H), 2.39 (s, 3H) ppm. | 285 |
| 6-9 | 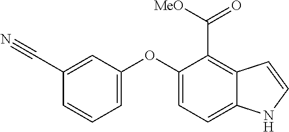 [SMILES: COC(=O)c1c(Oc2cccc(C#N)c2)ccc2[nH]ccc12] | methyl 5-(3- cyanophenoxy)-1H- indole-4-carboxylate | | 293 |
| 6-10 | 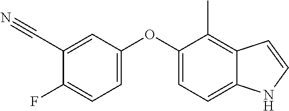 [SMILES: Cc1c(Oc2ccc(F)c(C#N)c2)ccc2[nH]ccc12] | 2-fluoro-5-((4-methyl- 1H-indol-5- yl)oxy)benzonitrile | | 267 |
| 6-11 | 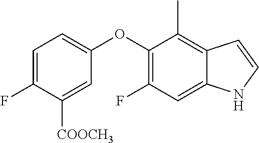 [SMILES: COC(=O)c1cc(Oc2c(F)cc3[nH]ccc3c2C)ccc1F] | methyl 2-fluoro-5-((6- fluoro-4-methyl-1H- indol-5-yl)oxy)benzoate | | 318 |
| 6-12 | 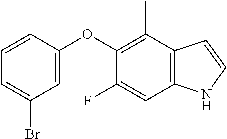 [SMILES: Cc1c(Oc2cccc(Br)c2)c(F)cc2[nH]ccc12] | 5-(3-bromophenoxy)-6- fluoro-4-methyl-1H- indole | | 320, 322 |
| 6-13 | 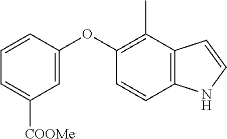 [SMILES: COC(=O)c1cccc(Oc2ccc3[nH]ccc3c2C)c1] | methyl 3-((4-methyl-1H- indol-5-yl)oxy)benzoate | | 282 |
| 6-14 | 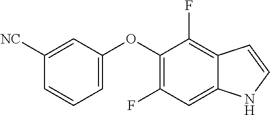 [SMILES: N#Cc1cccc(Oc2c(F)cc3[nH]ccc3c2F)c1] | 3-((4,6-difluoro-1H- indol-5- yl)oxy)benzonitrile | | 271 |

### Intermediate 7

### 5-((4-Bromo-6-fluoro-1-tosyl-1H-indol-5-yl)oxy)-2-fluorobenzonitrile

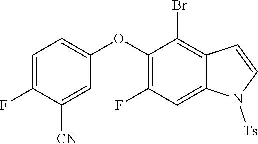 [SMILES: Cc1ccc(S(=O)(=O)n2ccc3c(Br)c(Oc4ccc(F)c(C#N)c4)c(F)cc32)cc1]

To a solution of 5-((4-bromo-6-fluoro-1H-indol-5-yl)oxy)-2-fluorobenzonitrile (Intermediate 6, 72 g, 264 mmol) in DMF (500 mL) was carefully added sodium hydride (12.7 g, 317 mmol, 60% purity) at room temperature. The reaction mixture was stirred for 30 minutes, 4-methylbenzenesulfonyl chloride (47.2 g, 247 mmol) was then added portion wise and continued stirring for 9.5 hours. The reaction was quenched with water (3 L) and extracted with ethyl acetate (2 L×2). The combined organic extracts were washed with brine (2 L), dried over sodium sulfate, filtered and concentrated to afford the title compound as a white solid (144 g). ¹H NMR: (400 MHz CDCl₃) δ 7.86 (d, J=10.4 Hz, 1H), 7.79 (d, J=8.0 Hz, 2H), 7.67 (d, J=2.8 Hz, 1H), 7.31 (d, J=8.0 Hz, 2H), 7.14 (d, J=6.8 Hz, 2H), 6.98 (s, 1H), 6.72 (d, J=2.8 Hz, 1H), 2.39 (s, 3H) ppm.

The following intermediates were prepared utilizing the procedures described for Intermediate 7.

| | | | | MS m/z |
|---|---|---|---|---|
| No | Structure | Name | ¹H NMR | [M + H]⁺ |
| 7-1 | 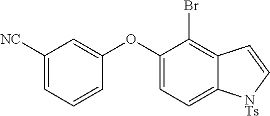 [SMILES: Cc1ccc(S(=O)(=O)n2ccc3c(Br)c(Oc4cccc(C#N)c4)ccc32)cc1] | 3-((4-bromo-1- tosyl-1H-indol-5- yl)oxy)benzonitrile | (400 MHz, CDCl₃) δ 7.97 (d, , J = 8.8 Hz, 1H), 7.80 (d, J = 8.2 Hz, 2H), 7.70 (d, J = 3.6 Hz, 1H), 7.42-7.26 (m, 4H), 7.16-7.12 (m, 1H), 7.06-7.03 (m, 2H), 6.77 (d, J = 3.6 Hz, 1H), 2.39 (s, 3H) ppm. | 467, 469 |
| 7-2 | 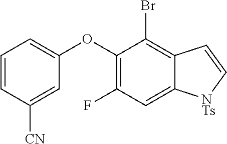 [SMILES: Cc1ccc(S(=O)(=O)n2ccc3c(Br)c(Oc4cccc(C#N)c4)c(F)cc32)cc1] | 3-((4-bromo-6- fluoro-1-tosyl-1H- indol-5- yl)oxy)benzonitrile | | 485, 487 |
| 7-3 | 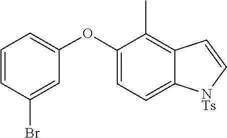 [SMILES: Cc1ccc(S(=O)(=O)n2ccc3c(C)c(Oc4cccc(Br)c4)ccc32)cc1] | 5-(3- bromophenoxy)-4- methyl-1-tosyl-1H- indole | | 456, 458 |
| 7-4 | 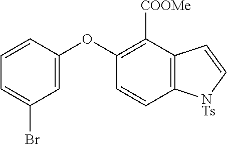 [SMILES: COC(=O)c1c(Oc2cccc(Br)c2)ccc2c1ccn2S(=O)(=O)c1ccc(C)cc1] | methyl 5-(3- bromophenoxy)-1- tosyl-1H-indole-4- carboxylate | | 500, 502 |
| 7-5 | 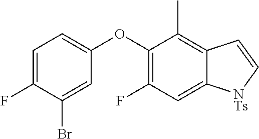 [SMILES: Cc1ccc(S(=O)(=O)n2ccc3c(C)c(Oc4ccc(F)c(Br)c4)c(F)cc32)cc1] | 5-(3-bromo-4- fluorophenoxy)-6- fluoro-4-methyl-1- tosyl-1H-indole | (400 MHz, CDCl₃) 7.79 (d, 2H), 7.71 (d, 1H), 7.59 (d, 1H), 7.29 (m, 2H), 6.96- 6.03 (m, 2H), 6.73-6.33 (m, 1H), 6.66 (d, 1H), 2.39 (s, 3H), 2.30 (s, 3H) ppm. | |
| 7-6 | 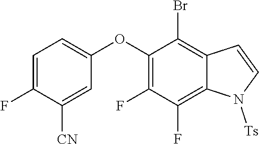 [SMILES: Cc1ccc(S(=O)(=O)n2ccc3c(Br)c(Oc4ccc(F)c(C#N)c4)c(F)c(F)c32)cc1] | 5-((4-bromo-6,7- difluoro-1-tosyl-1H- indol-5-yl)oxy)-2- fluorobenzonitrile | (400 MHz, CD₃OD) δ 7.91-7.90 (d, J = 4 Hz, 1H), 7.88-7.87 (d, J = 4 Hz, 1H), 7.36-7.34 (d, J = 8 Hz, 2H), 7.17-7.15 (m, 3H), 7.01-7.00 (m, 1H), 6.78-6.77 (m, 1H), 2.43 (s, 3H) ppm. | |
| 7-7 | 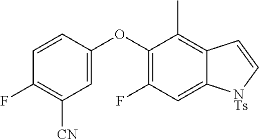 [SMILES: Cc1ccc(S(=O)(=O)n2ccc3c(C)c(Oc4ccc(F)c(C#N)c4)c(F)cc32)cc1] | 2-fluoro-5-((6- fluoro-4-methyl-1- tosyl-1H-indol-5- yl)oxy)benzonitrile | (400 MHz, CDCl₃) δ 7.80 (d, J = 8.3 Hz, 2H), 7.72 (d, J = 10.4 Hz, 1H), 7.61 (d, J = 3.7 Hz, 1H), 7.29 (d, J = 8.1 Hz, 2H), 7.14-7.10 (m, 2H), 6.93 (d, J = 4.1 Hz, 1H), 6.67 (d, J = 3.7 Hz, 1H), 2.39 (s, 3H), 2.30 (s, 3H) ppm. | 461 m/z [M + Na]⁺. |
| 7-8 | 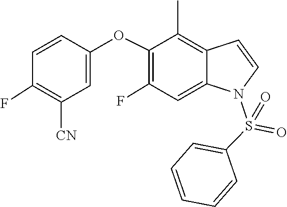 [SMILES: Cc1c(Oc2ccc(F)c(C#N)c2)c(F)cc2c1ccn2S(=O)(=O)c1ccccc1] | 2-fluoro-5-((6- fluoro-4-methyl-1- (phenylsulfonyl)- 1H-indol-5- yl)oxy)benzonitrile | | 425 |
| 7-9 | 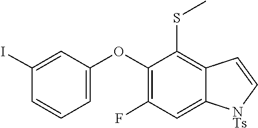 [SMILES: CSc1c(Oc2cccc(I)c2)c(F)cc2c1ccn2S(=O)(=O)c1ccc(C)cc1] | 6-fluoro-5-(3- iodophenoxy)-4- (methylthio)-1- tosyl-1H-indole | (400 MHz, CDCl₃) δ 7.22 (m, 3H), 7.62 (d, J = 3.6 Hz, 1H), 7.36 (d, J = 8.0 Hz, 1H), 7.30 (d, J = 8.0 Hz, 2H), 7.19 (s, 1H), 7.00-6.96 (m, 1 H), 6.87 (d, J = 3.6 Hz, 1H), 6.80-6.78 (m, 1 H), 2.39 (s, 3H), 2.38 (s, 3H) ppm | |

### Intermediate 8

### 2-Fluoro-5-((6-fluoro-1-tosyl-4-vinyl-1H-indol-5-yl)oxy)benzonitrile

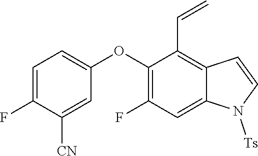 [SMILES: C=Cc1c(Oc2ccc(F)c(C#N)c2)c(F)cc2c1ccn2S(=O)(=O)c1ccc(C)cc1]

To a suspension of 5-((4-bromo-6-fluoro-1-tosyl-1H-indol-5-yl)oxy)-2-fluorobenzonitrile (Intermediate 7, 5.25 g, 10.5 mmol) in dioxane (120 mL) and water (40 mL) were added 4,4,5,5-tetramethyl-2-vinyl-1,3,2-dioxaborolane (3.2 g, 21 mmol), Pd(dppf)Cl₂ (384 mg, 0.5 mmol) and cesium carbonate (6.8 g, 21 mmol). The reaction mixture was stirred at 100° C. overnight, cooled to room temperature and extracted with ethyl acetate (150 mL×3). The combined organic extracts were washed with brine, dried over magnesium sulfate and evaporated to dryness. The resulting residue was purified by flash chromatography over silica (petroleum ether/dichloromethane, v/v, 2/1) to afford the title compound as a yellow solid (4 g, 85%). MS: 473 m/z [M+Na]⁺.

The following intermediates were prepared utilizing the procedures described for Intermediate 8.

| | | | MS m/z |
|---|---|---|---|
| No | Structure | Name | [M + H]⁺ |
| 8-1 | 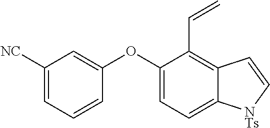 [SMILES: C=Cc1c(Oc2cccc(C#N)c2)ccc2c1ccn2S(=O)(=O)c1ccc(C)cc1] | 3-((1-tosyl-4-vinyl-1H-indol-5- yl)oxy)benzonitrile | 415 |
| 8-2 | 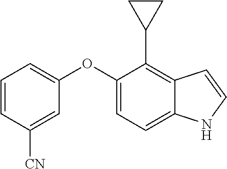 [SMILES: N#Cc1cccc(Oc2ccc3[nH]ccc3c2C2CC2)c1] | 3-((4-cyclopropyl-1H-indol-5- yl)oxy)benzonitrile | 275 |
| 8-3 | 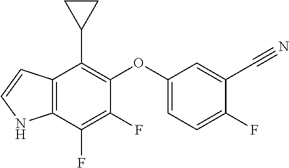 [SMILES: N#Cc1cc(Oc2c(F)c(F)c3[nH]ccc3c2C2CC2)ccc1F] | 5-((4-cyclopropyl-6,7-difluoro-1H- indol-5-yl)oxy)-2-fluorobenzonitrile | 329 |

### Intermediate 9

### 2-Fluoro-5-((6-fluoro-4-formyl-1-tosyl-1H-indol-5-yl)oxy)benzonitrile

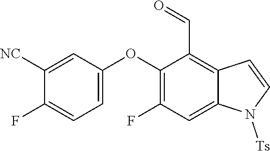 [SMILES: Cc1ccc(S(=O)(=O)n2ccc3c(C=O)c(Oc4ccc(F)c(C#N)c4)c(F)cc32)cc1]

To a solution of 2-fluoro-5-((6-fluoro-1-tosyl-4-vinyl-1H-indol-5-yl)oxy)benzonitrile (Intermediate 8, 4.6 g, 10.2 mmol) in THF (90 mL) were added 2,6-lutidine (1.1 g, 10.2 mmol) and osmium tetroxide (2 mL saturated in water) at 0° C. The reaction mixture was stirred for three minutes and a solution of sodium periodate (8.8 g, 4.0 mmol) in water (30 mL) was added. The reaction mixture was stirred at room temperature overnight, acidified with 2 M hydrochloric acid (100 mL) and extracted with ethyl acetate (200 mL×3). The combined organic extracts were washed with water, brine, dried over magnesium sulfate and evaporated to dryness. The resulting residue was purified by flash chromatography over silica (petroleum ether/ethyl acetate, v/v, 10/1) to afford the title compound as a yellow solid (3.5 g, 75%). MS: 453 m/z [M+H]⁺.

The following intermediates were prepared utilizing the procedures described for Intermediate 9.

| | | | MS m/z |
|---|---|---|---|
| No. | Structure | Name | [M + H]⁺ |
| 9-1 | 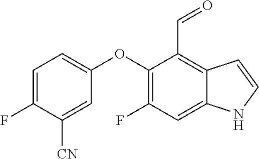 [SMILES: N#Cc1cc(Oc2c(F)cc3[nH]ccc3c2C=O)ccc1F] | 2-fluoro-5-((6-fluoro-4-formyl-1H-indol-5- yl)oxy)benzonitrile | 299 |

### Intermediate 10

### 5-((4-(2,2-Difluoroethyl)-6-fluoro-1H-indol-5-yl)oxy)-2-fluorobenzonitrile

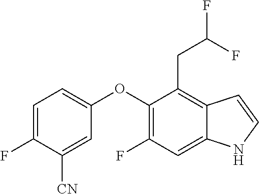 [SMILES: N#Cc1cc(Oc2c(F)cc3[nH]ccc3c2CC(F)F)ccc1F]

A mixture of 5-((4-(2,2-difluoroethyl)-6-fluoro-1-tosyl-1H-indol-5-yl)oxy)-2-fluorobenzonitrile (Intermediate 10B, 1.38 g, 2.83 mmol) and potassium carbonate (1.17 g, 8.48 mmol) in methanol (15 mL) was stirred at 80° C. for forty five minutes in a microwave reactor. The reaction mixture was diluted with water (50 mL) and extracted with dichloromethane (50 mL×3). The combined organic extracts were washed with water and brine, dried over magnesium sulfate and evaporated to dryness. The resulting residue was purified by flash chromatography over silica (0-50% dichloromethane/petroleum ether) to afford 5-((4-(2,2-difluoroethyl)-6-fluoro-1H-indol-5-yl)oxy)-2-fluorobenzonitrile as a white solid (0.65 g, 69%). MS: 335 m/z [M+H]⁺.

### Intermediate 10A

### 5-((4-(2,2-Difluorovinyl)-6-fluoro-1-tosyl-1H-indol-5-yl)oxy)-2-fluorobenzonitrile

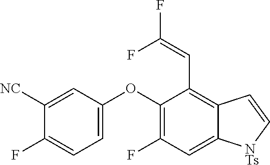 [SMILES: Cc1ccc(S(=O)(=O)n2ccc3c(C=C(F)F)c(Oc4ccc(F)c(C#N)c4)c(F)cc32)cc1]

A solution of 2-fluoro-5-((6-fluoro-4-formyl-1-tosyl-1H-indol-5-yl)oxy)benzonitrile (Intermediate 9, 2.5 g, 5.53 mmol), sodium chlorodifluoroacetate (2.52 g, 5.53 mmol), and triphenylphosphine (1.45 g, 5.53 mmol) in DMF (25 mL) was heated at 90° C. for two hours. The reaction mixture was cooled to room temperature, poured into water (30 mL) and extracted with ethyl acetate (20 mL×3). The combined organic extracts were washed with brine, dried over magnesium sulfate, filtered, and concentrated to dryness. The resulting residue was purified by silica gel chromatography (0-50% dichloromethane in petroleum ether) to afford the title compound as a white solid (1.5 g, 56%). MS: 509 m/z [M+Na]⁺. ¹H NMR (400 MHz, CDCl₃) δ 7.80-7.86 (m, 3H), 7.66 (d, J=3.6 Hz, 1H), 7.30-7.33 (m, 2H), 7.09-7.16 (m, 2H), 6.95-6.96 (m, 1H), 6.63 (m, 1H), 5.34 (m, 1H), 2.40 (s, 3H) ppm.

### Intermediate 10B

### 5-((4-(2,2-Difluoroethyl)-6-fluoro-1-tosyl-1H-indol-5-yl)oxy)-2-fluorobenzonitrile

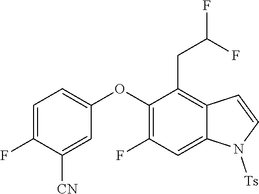 [SMILES: Cc1ccc(S(=O)(=O)n2ccc3c(CC(F)F)c(Oc4ccc(F)c(C#N)c4)c(F)cc32)cc1]

To a degassed solution of 5-((4-(2,2-difluorovinyl)-6-fluoro-1-tosyl-1H-indol-5-yl)oxy)-2-fluorobenzonitrile (Intermediate 10A, 1.5 g, 3.09 mmol) in ethyl acetate (25 mL) was added Pd/C (0.2 g). The reaction mixture was stirred under a hydrogen balloon at room temperature for 45 minutes, filtered and concentrated to dryness. The resulting residue was purified by silica gel chromatography (0-50% dichloromethane in petroleum ether) to afford the title compound as a white solid (1.38 g, 92%). MS: 511 m/z [M+Na]⁺. ¹H NMR (400 MHz, CDCl₃) δ 7.81-7.86 (m, 3H), 7.66 (d, J=3.6 Hz, 1H), 7.30-7.33 (m, 2H), 7.13-7.15 (m, 2H), 6.98-6.99 (m, 1H), 6.70 (d, J=3.6 Hz, 1H), 5.78-6.09 (m, 1H), 3.27-3.37 (m, 2H), 2.40 (s, 3H) ppm.

### Intermediate 11

### 2-Fluoro-5-((6-fluoro-4-(2,2,2-trifluoroethyl)-1H-indol-5-yl)oxy)benzonitrile

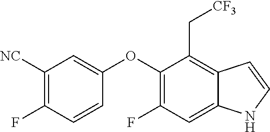 [SMILES: N#Cc1cc(Oc2c(F)cc3[nH]ccc3c2CC(F)(F)F)ccc1F]

A mixture of 2-fluoro-5-((6-fluoro-1-tosyl-4-(2,2,2-trifluoroethyl)-1H-indol-5-yl)oxy)benzonitrile (Intermediate 11A, 160 mg, 0.32 mmol) and potassium carbonate (131 mg, 0.95 mmol) in methanol (5 mL) was stirred at 80° C. for 30 minutes in a microwave reactor. The reaction mixture was diluted with water (50 mL) and extracted with dichloromethane (50 mL×3). The combined organic extracts were washed with brine, dried over magnesium sulfate and evaporated to dryness. The resulting residue was purified by flash chromatography over silica (0-20% ethyl acetate in petroleum) to give the title compound (80 mg, 72%) as a white foam. MS: 353 m/z [M+H]⁺. ¹H NMR (400 MHz, CDCl₃) δ 8.37 (br, 1H), 7.33 (dd, J=2.4 Hz, 1H), 7.25-7.27 (m, 1H), 7.17-7.20 (m, 1H), 7.11-7.15 (m, 1H), 7.03 (dd, J=4.0 Hz, 1H), 6.62 (m, 1H), 3.69 (m, 2H) ppm.

### Intermediate 11A

### 2-Fluoro-5-((6-fluoro-1-tosyl-4-(2,2,2-trifluoroethyl)-1H-indol-5-yl)oxy)benzonitrile

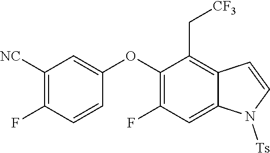 [SMILES: Cc1ccc(S(=O)(=O)n2ccc3c(CC(F)(F)F)c(Oc4ccc(F)c(C#N)c4)c(F)cc32)cc1]

A mixture of 5-((4-(2,2-difluorovinyl)-6-fluoro-1-tosyl-1H-indol-5-yl)oxy)-2-fluorobenzonitrile (Intermediate 10A, 250 mg, 0.51 mmol), potassium fluoride (90 mg, 1.54 mmol) in DMF (5 mL) and five drops of water was degassed with nitrogen and heated at 70° C. for two hours. The reaction mixture was cooled to room temperature, diluted with water (20 mL) and extracted with ethyl acetate (20 mL×3). The combined organic extracts were washed with brine, dried over sodium sulfate and evaporated to dryness. The resulting residue was purified by flash chromatography over silica (20-30% ethyl acetate in petroleum ether) to give the title compound as a white foam (160 mg, 66%). MS: 507 m/z [M+H]⁺, 529 m/z [M+Na]⁺. ¹H NMR (400 MHz, CDCl₃) δ 7.89 (d, J=10.4 Hz, 1H), 7.81-7.84 (m, 2H), 7.69 (d, J=3.6 Hz, 1H), 7.31-7.36 (m, 2H), 7.13-7.15 (m, 2H), 7.00-7.02 (m, 1H), 6.71 (d, J=4 Hz, 1H), 2.63 (m, 2H), 2.41 (s, 3H) ppm.

### Intermediate 12

### 5-((4-Chloro-6-fluoro-1H-indol-5-yl)oxy)-2-fluorobenzonitrile

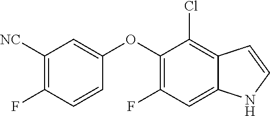 [SMILES: N#Cc1cc(Oc2c(F)cc3[nH]ccc3c2Cl)ccc1F]

5-(3-Bromo-4-fluorophenoxy)-4-chloro-6-fluoro-1H-indole (Intermediate 12C, 108 mg, 0.3 mmol), Zn(CN)₂ (70 mg, 0.6 mmol), Zn (2 mg, 0.03 mmol), DMF (2 mL) and Pd(dppf)Cl₂ (50 mg, 0.06 mmol) were added to a 2 mL microwave vial, purged with nitrogen and heated at 160° C. in a microwave reactor for one hour. The reaction mixture was diluted with ethyl acetate (60 mL), washed with water (15 mL×2) and brine, dried over sodium sulfate, filtered and concentrated. The resulting residue was purified by flash chromatography over silica (0-30% ethyl acetate in petroleum ether) to afford the title compound as a white solid (58 mg, 64%). ¹H NMR (400 MHz, CDCl₃) δ 8.40 (s, 1H), 7.32 (t, 1H), 7.20-7.24 (m, 2H), 7.15 (t, 1H), 7.03 (t, 1H), 6.82 (t, 1H) ppm.

### Intermediate 12A

### 3-chloro-1,2-difluoro-4-methyl-5-nitrobenzene

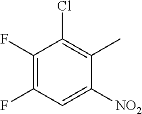

A 20 mL microwave tube containing 3-bromo-1,2-difluoro-4-methyl-5-nitrobenzene (Intermediate 6A, 0.5 g, 2 mmol), CuCl (1.2 g, 12 mmol) and DMF (5 mL) was heated at 160° C. for 1.5 hours in a microwave reactor. The reaction mixture was diluted with ethyl acetate (100 mL) and then filtered. The filtrate was washed with water (30 mL) and brine (25 mL), dried over sodium sulfate, filtered and concentrated to afford 3-chloro-1,2-difluoro-4-methyl-5-nitrobenzene as a yellow oil (0.4 g, 96%). MS: 208 m/z [M+H]⁺.

### Intermediate 12B

### 2-(3-Bromo-4-fluorophenoxy)-3-chloro-1-fluoro-4-methyl-5-nitrobenzene

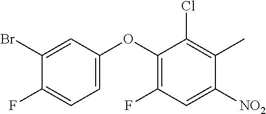 [SMILES: Cc1c([N+](=O)[O-])cc(F)c(Oc2ccc(F)c(Br)c2)c1Cl]

To a solution of 3-chloro-1,2-difluoro-4-methyl-5-nitrobenzene (Intermediate 12A, 0.39 g, 1.9 mmol) and 3-bromo-4-fluorophenol (0.4 g, 2.1 mmol) in DMF (15 mL) was added cesium carbonate (1.24 g, 3.8 mmol). The reaction mixture was stirred at room temperature overnight, diluted with water (75 mL) and extracted with ethyl acetate (30 mL×3). The combined extracts were washed with water (20 mL), brine (15 mL) and concentrated. The resulting residue was purified by flash chromatography over silica (0-2% ethyl acetate in petroleum ether) to afford the title compound as a yellow oil (0.57 g, 80%). ¹H NMR (400 MHz, CDCl₃) δ 7.71 (d, 1H), 7.06-7.11 (m, 2H), 6.83-6.87 (m, 1H), 2.61 (s, 3H) ppm.

### Intermediate 12C

### 5-(3-Bromo-4-fluorophenoxy)-4-chloro-6-fluoro-1H-indole

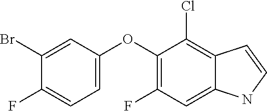

To a solution of 2-(3-bromo-4-fluorophenoxy)-3-chloro-1-fluoro-4-methyl-5-nitrobenzene (Intermediate 12B, 0.26 g, 0.7 mmol) in DMF (10 mL) was added DMF-DMA (0.92 mL, 7 mmol). The reaction mixture was stirred at 100° C. for three hours and concentrated to dryness. The resulting residue was dissolved in acetic acid (10 mL) and toluene (10 mL) and iron powder (0.39 g, 7 mmol) was added. The mixture was stirred at 100° C. overnight, cooled to room temperature, filtered through Celite and concentrated. The residue was dissolved in ethyl acetate (50 mL), washed with water (30 mL), and brine (20 mL), dried over sodium sulfate, filtered and evaporated to dryness. The resulting residue was purified by silica gel column chromatography (petroleum ether/ethyl acetate, v/v, 10/1 to 4/1) to afford the title compound as a yellow oil (120 mg, 48%). ¹H NMR (400 MHz, CDCl₃) δ 8.34 (s, 1H), 7.30 (t, 1H), 7.18 (d, 1H), 7.01-7.08 (m, 2H), 6.84-6.88 (m, 1H), 6.68 (t, 1H) ppm.

### Intermediate 13

### 2-(3-Bromo-2-cyanophenyl)acetic acid

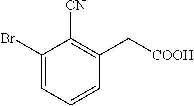 [SMILES: N#Cc1c(Br)cccc1CC(=O)O]

To a solution of diethyl 2-(3-bromo-2-cyanophenyl)malonate (Intermediate 13A, 23 g, 67.6 mmol) in methanol (100 mL) and water (100 mL) was added sodium hydroxide (27 g, 676 mmol). The reaction mixture was stirred at room temperature overnight under N₂, concentrated to remove methanol and extracted with ethyl acetate (50 mL) to remove any neutral impurity. The aqueous layer was acidified with 6 N HCl, to pH-2 and extracted with ethyl acetate (3×200 mL). The combined organic extracts were dried over sodium sulfate, filtered and concentrated. The resulting residue was purified by flash silica gel column chromatography (petroleum ether/ethyl acetate, v/v, 3/1) to afford the title compound as a yellow solid (12.8 g, 78%). ¹H NMR (400 MHz, CDCl₃) δ 7.63 (d, J=8.0 Hz, 1H), 7.43 (t, J=7.6 Hz, 1H), 7.37 (d, J=7.6 Hz, 1H), 3.95 (s, 2H) ppm.

### Intermediate 13A

### Diethyl 2-(3-bromo-2-cyanophenyl)malonate

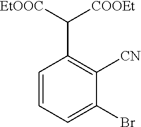 [SMILES: CCOC(=O)C(C(=O)OCC)c1cccc(Br)c1C#N]

To a solution of 2-bromo-6-fluorobenzonitrile (20 g, 100 mmol) in DMSO (100 mL) were added cesium carbonate (66 g, 200 mmol) and diethyl malonate (32 g, 200 mmol). The reaction mixture was heated at 100° C. for four hours under N₂, diluted with water (200 mL) and extracted with ethyl acetate (3×200 mL). The combined organic extracts were dried over sodium sulfate, filtered, and concentrated. The resulting residue was purified by flash silica gel column chromatography (petroleum ether/ethyl acetate, v/v, 10/1) to afford the title compound as a yellow oil (24 g, 70%). ¹H NMR (400 MHz, CDCl₃) δ 7.67 (d, J=3.2 Hz, 1H), 7.66 (d, J=3.2 Hz, 1H), 7.48 (t, J=8.0 Hz, 1H), 5.14 (s, 1H), 4.28-4.24 (m, 4H), 1.29 (t, J=6.8 Hz, 6H) ppm.

### Intermediate 14

### 3-((4-Vinyl-1H-indol-5-yl)oxy)benzonitrile

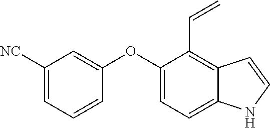 [SMILES: C=Cc1c(Oc2cccc(C#N)c2)ccc2[nH]ccc12]

A mixture of 3-((1-tosyl-4-vinyl-1H-indol-5-yl)oxy)benzonitrile (Intermediate 8-1, 3 g, 7.25 mmol) and potassium carbonate (3 g, 21.74 mmol) in methanol (30 mL) was refluxed for 1.5 hours, cooled to room temperature and concentrated. The resulting residue was diluted with ethyl acetate (100 mL), washed with water and brine, dried over sodium sulfate, filtered and concentrated. The resulting residue was purified by flash chromatography over silica (petroleum ether/ethyl acetate, v/v, 10/1-5/1) to afford the title compound as a light yellow oil (1.4 g, 74%). MS: 261 m/z [M+H]⁺.

The following intermediate was prepared utilizing the procedure described for Intermediate 14.

| | | | MS m/z |
|---|---|---|---|
| No. | Structure | Name | [M + H]⁺ |
| 14-1 | 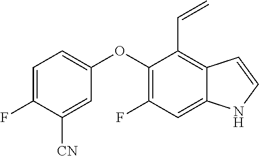 [SMILES: C=Cc1c(Oc2ccc(F)c(C#N)c2)c(F)cc2[nH]ccc12] | 2-fluoro-5-((6-fluoro-4-vinyl-1H- indol-5-yl)oxy)benzonitrile | 297 |

### Intermediate 15

### 3-((6-Fluoro-4-methyl-1H-indol-5-yl)oxy)benzonitrile

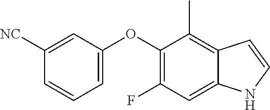 [SMILES: Cc1c(Oc2cccc(C#N)c2)c(F)cc2[nH]ccc12]

A mixture of 3-((4-bromo-6-fluoro-1H-indol-5-yl)oxy)benzonitrile (Intermediate 6-4, 600 mg, 1.82 mmol), methylboronic acid (1.1 g, 18.2 mmol), Pd(dppf)Cl₂ (300 mg, 0.46 mmol) and sodium bicarbonate (460 mg, 5.45 mmol) in dioxane (50 mL) and water (10 mL) was heated at 80° C. overnight. The reaction mixture was cooled to room temperature, diluted with dichloromethane (50 mL), washed with saturated sodium bicarbonate (10 mL×3) and brine, dried over sodium sulfate, filtered and concentrated. The crude mixture was purified by flash chromatography over silica (petroleum ether/dichloromethane, v/v, 1:1) to give the title compound as a white solid (360 mg, 74%). MS: 267 m/z [M+H]⁺.

### Intermediate 16

### 3-((6-Fluoro-4-(thiazol-2-yl)-1H-indol-5-yl)oxy)benzonitrile

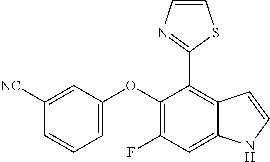 [SMILES: N#Cc1cccc(Oc2c(F)cc3[nH]ccc3c2-c2nccs2)c1]

To a solution of 3-((6-fluoro-4-(thiazol-2-yl)-1-tosyl-1H-indol-5-yl)oxy)benzonitrile (Intermediate 16A, 1.6 g, 3.27 mmol) in THF (20 mL) was added tetrabutylammonium fluoride (1M in THF, 13.1 mL, 13.1 mmol). The reaction mixture was stirred at 40° C. for 16 hours, quenched with aqueous saturated ammonium chloride (40 mL) and extracted by ethyl acetate (80 mL×2). The combined organic extracts were washed with brine, dried over sodium sulfate, filtered and concentrated. The resulting residue was purified by flash chromatography over silica (dichloromethane/methanol, v/v, 20/1) to give the title compound as a yellow solid (636 mg, 58%). MS: 336 m/z [M+H]⁺.

### Intermediate 16A

### 3-((6-Fluoro-4-(thiazol-2-yl)-1-tosyl-1H-indol-5-yl)oxy)benzonitrile

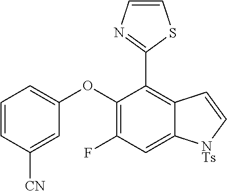 [SMILES: Cc1ccc(S(=O)(=O)n2ccc3c(-c4nccs4)c(Oc4cccc(C#N)c4)c(F)cc32)cc1]

A mixture of 3-((4-bromo-6-fluoro-1-tosyl-1H-indol-5-yl)oxy)benzonitrile (Intermediate 7-2, 2 g, 4.12 mmol), 2-(tributylstannyl)thiazole (3.08 g, 8.24 mmol) and Pd(PPh₃)₂Cl₂ (579 mg, 0.82 mmol) in DMF (20 mL) was stirred at 100° C. for three hours under nitrogen atmosphere. The reaction mixture was concentrated in vacuo, diluted with ethyl acetate (150 mL), washed with water (150 mL), brine (150 mL), dried over sodium sulfate, filtered and concentrated. The resulting residue was purified by flash chromatography over silica (petroleum ether/ethyl acetate/dichloromethane, v/v, 10/1/1) to afford the title compound as a yellow solid (1.3 g, 65%). MS: 490 m/z [M+H]⁺.

### Intermediate 17

### 2-Fluoro-5-((6-fluoro-1H-pyrrolo[3,2-b]pyridin-5-yl)oxy)benzonitrile

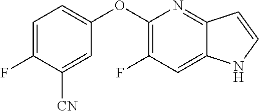 [SMILES: N#Cc1cc(Oc2nc3cc[nH]c3cc2F)ccc1F]

To a solution of 5-((5-amino-3-fluoro-6-((trimethylsilyl)ethynyl)pyridin-2-yl)oxy)-2-fluorobenzonitrile (Intermediate 17D, 450 mg, 1.31 mmol) in DMF (6 mL) was added CuI (499 mg, 2.62 mmol) in glove box and stirred at 100° C. for three hours. The reaction mixture was cooled to 0° C., poured into water (50 mL), diluted with a 3:1 mixture of ethyl acetate and THF (100 mL), filtered through a pad of Celite and the filter cake was washed with ethyl acetate (50 mL×2). The combined filtrate was washed with water and brine (100 mL), dried over sodium sulfate, filtered and concentrated. The resulting residue was purified by flash chromatography over silica (petroleum ether/ethyl acetate, v/v, 1/2) to afford the title compound (180 mg, 53%). MS: 272 m/z [M+H]⁺.

### Intermediate 17A

### 2-Fluoro-5-((3-fluoro-5-nitropyridin-2-yl)oxy)benzonitrile

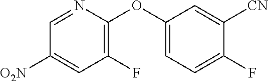 [SMILES: N#Cc1cc(Oc2ncc([N+](=O)[O-])cc2F)ccc1F]

To a solution of 2-chloro-3-fluoro-5-nitropyridine (2.28 g, 13.0 mmmol) in DMF (15 mL) were added 2-fluoro-5-hydroxybenzonitrile (1.77 g, 13 mmol) and potassium carbonate (3.59 g, 26 mmol). The reaction mixture was stirred at 100° C. for two hours, cooled to room temperature, diluted with water and extracted with ethyl acetate (50 mL×2). The combined organic extracts were washed with brine (50 mL×2), dried over sodium sulfate and concentrated. The resulting residue was purified by flash silica gel column chromatography (petroleum ether/ethyl acetate, v/v, 3/1) to give the title compound as a yellow solid (1.84 g, 52%). MS: 278 m/z [M+H]⁺. ¹H NMR (400 MHz, DMSO-d₆) δ 8.90 (d, J=2.4 Hz, 111), 8.84 (dd, J=9.6, 2.4 Hz, 1H), 8.06 (dd, J=5.3, 2.9 Hz, 1H), 7.86-7.78 (m, 1H), 7.69 (t, J=9.0 Hz, 1H) ppm.

### Intermediate 17B

### 5-((5-Amino-3-fluoropyridin-2-yl)oxy)-2-fluorobenzonitrile

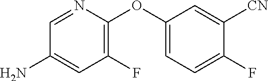 [SMILES: N#Cc1cc(Oc2ncc(N)cc2F)ccc1F]

To a solution of 2-fluoro-5-((3-fluoro-5-nitropyridin-2-yl)oxy)benzonitrile (Intermediate 17A, 1.84 g, 6.6 mmol) in 30 mL of ethanol/water (v/v, 1/1) were added iron powder (1.49 g, 26.6 mmol) and ammonium chloride (2.8 g, 52.8 mmol). The reaction mixture was stirred at 80° C. for four hours, cooled to room temperature, diluted with water (30 mL) and ethyl acetate (100 mL), filtered through a pad of Celite and the filter cake was washed with THF/ethyl acetate (v,v, 1/2, 50 mL×4). The filtrate was washed with brine (100 mL), dried over sodium sulfate, filtered and concentrated to give the title compound as a yellow solid (1.6 g, 97%), which was directly used in the next step without further purification. MS: 248 m/z [M+H]⁺. ¹H NMR (400 MHz, DMSO-d₆) δ 7.67 (dd, J=5.2, 3.0 Hz, 11), 7.52 (t, J=9.0 Hz, 1H), 7.47-7.42 (m, 1H), 7.38 (d, J=2.3 Hz, 1H), 7.02 (dd, J=12.2, 2.4 Hz, 1H), 5.53 (s, 2H) ppm.

### Intermediate 17C

### 5-((5-Amino-3-fluoro-6-iodopyridin-2-yl)oxy)-2-fluorobenzonitrile

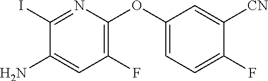 [SMILES: N#Cc1cc(Oc2nc(I)c(N)cc2F)ccc1F]

To a solution of 5-((5-amino-3-fluoropyridin-2-yl)oxy)-2-fluorobenzonitrile (Intermediate 17B, 1 g, 4 mmol) in acetic acid (10 mL) was added NIS (910 mg, 4 mmol) below 10° C. The reaction mixture was stirred at room temperature for one hour, diluted with ethyl acetate (100 mL), washed with brine (30 mL), saturated sodium bicarbonate (30 mL×3) and brine (30 mL) again, dried over sodium sulfate, filtered and concentrated. The resulting residue was purified by flash chromatography over silica (40-60% ethyl acetate in petroleum ether) to give the title compound as a yellow solid (1.2 g, 80%). MS: 374.0 m/z [M+H]⁺. ¹H NMR (400 MHz, DMSO-d₆) δ 7.75 (dd, J=5.2, 2.8 Hz, 1H), 7.60-7.46 (m, 2H), 7.10 (d, J=11.7 Hz, 1H), 5.56 (s, 2H) ppm.

### Intermediate 17D

### 5-((5-Amino-3-fluoro-6-((trimethylsilyl)ethynyl)pyridin-2-yl)oxy)-2-fluorobenzonitrile

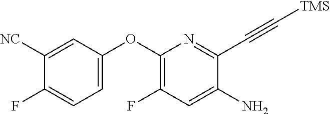 [SMILES: C[Si](C)(C)C#Cc1nc(Oc2ccc(F)c(C#N)c2)c(F)cc1N]

A solution of 5-((5-amino-3-fluoro-6-iodopyridin-2-yl)oxy)-2-fluorobenzonitrile (Intermediate 17C, 750 mg, 2 mmol), ethynyltrimethylsilane (235 mg, 2.4 mmol), Pd(PPh₃)₂Cl₂ (140 mg, 0.2 mmol), CuI (38 mg, 0.2 mmol) and triethylamine (0.5 mL, 3.6 mmol) in DMF (6 mL) was stirred at 30° C. for three hours, cooled to 0° C., poured into ice water (50 mL) and extracted with ethyl acetate (30 mL×2). The combined organic extracts were washed with brine (60 mL), dried over sodium sulfate, filtered and concentrated. The resulting residue was purified by chromatography on silica gel (petroleum ether/ethyl acetate, v/v, 3/1) to give the title compound as a light yellow solid (450 mg, 65%). MS: 344 m/z [M+H]⁺. ¹H NMR (400 MHz, DMSO-d₆) δ 7.75 (dd, J=5.1, 2.8 Hz, 1H), 7.58-7.47 (m, 2H), 7.19 (d, J=11.5 Hz, 1H), 5.67 (s, 2H), 0.22 (s, 9H) ppm.

### Intermediate 18

### 5-((4-(Difluoromethyl)-6-fluoro-1H-indol-5-yl)oxy)-2-fluorobenzonitrile

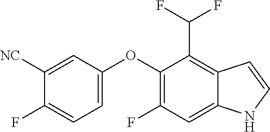 [SMILES: N#Cc1cc(Oc2c(F)cc3[nH]ccc3c2C(F)F)ccc1F]

To a solution of 5-((4-(difluoromethyl)-6-fluoro-1-tosyl-1H-indol-5-yl)oxy)-2-fluorobenzonitrile (Intermediate 18A, 580 mg, 1.22 mmol) in THF (5 mL) was added tetrabutylammonium fluoride (1M in THF, 6.2 mL). The reaction mixture was refluxed for one hour, cooled to room temperature, diluted with ethyl acetate (100 mL), washed with brine, dried over magnesium sulfate and evaporated to dryness. The resulting residue was purified by flash chromatography over silica (20-50% ethyl acetate in petroleum ether) to give the title compound as a white solid (320 mg, 82%). MS: 321 m/z [M+H]⁺.

### Intermediate 18A

### 5-((4-(Difluoromethyl)-6-fluoro-1-tosyl-1H-indol-5-yl)oxy)-2-fluorobenzonitrile

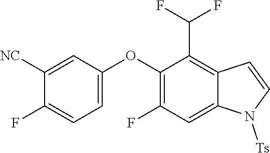 [SMILES: Cc1ccc(S(=O)(=O)n2ccc3c(C(F)F)c(Oc4ccc(F)c(C#N)c4)c(F)cc32)cc1]

To a solution of 2-fluoro-5-((6-fluoro-4-formyl-1-tosyl-1H-indol-5-yl)oxy)benzonitrile (Intermediate 9, 700 mg, 1.55 mmol) in dichloromethane (2 mL) was added DAST (1.25 g, 7.74 mmol) at 0° C. The mixture was stirred for two hours, pH adjusted to ˜8 with saturated sodium bicarbonate and extracted with dichloromethane (50 mL×2). The combined organic extracts were dried, filtered and evaporated. The resulting residue was purified by flash chromatography over silica (petroleum ether/ethyl acetate, v/v, 5/1) to give the title compound as a white solid (680 mg, 92%). ¹H NMR (400 MHz, CDCl₃) δ 8.02 (d, J=10.4 Hz, 1H), 7.80-7.82 (m, 2H), 7.72 (d, J=3.6 Hz, 1H), 7.31-7.33 (m, 2H), 6.97-7.16 (m, 5H), 2.406 (s, 3H) ppm.

### Intermediate 19

### 2-Fluoro-5-((6-fluoro-4-(S-methylsulfonimidoyl)-1H-indol-5-yl)oxy)benzonitrile

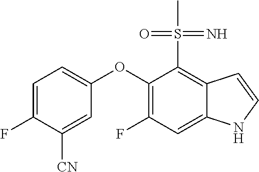 [SMILES: CS(=N)(=O)c1c(Oc2ccc(F)c(C#N)c2)c(F)cc2[nH]ccc12]

N-((5-(3-cyano-4-fluorophenoxy)-6-fluoro-1-tosyl-1H-indol-4-yl)(methyl)(oxo)-6-sulfaneylidene)-2,2,2-trifluoroacetamide (Intermediate 19B, 570 mg, 0.95 mmol) was dissolved in tetrabutylammonium fluoride (1M in THF, 15 mL). The reaction mixture was stirred at 70° C. for four hours, diluted with ethyl acetate (50 mL), washed with water (30 mL×2) and brine (20 mL×3), dried over sodium sulfate, filtered and concentrated. The resulting residue was purified by flash silica gel column chromatography (0-70% ethyl acetate in petroleum ether) to give the title compound as a solid (240 mg, 73%). MS: 348 m/z [M+H]⁺. ¹H NMR (400 MHz, CDCl₃) δ 8.70 (s, 1H), 7.49 (d, J=9.8 Hz, 1H), 7.46-7.39 (m, 2H), 7.24-7.11 (m, 2H), 7.05 (dd, J=4.7, 3.1 Hz, 1H), 3.37 (s, 3H), 3.06 (s, 1H) ppm.

### Intermediate 19A

### 2-Fluoro-5-((6-fluoro-4-(methylsulfinyl)-1-tosyl-1H-indol-5-yl)oxy)benzonitrile

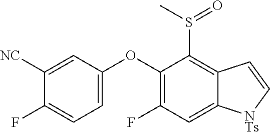 [SMILES: Cc1ccc(S(=O)(=O)n2ccc3c(S(C)=O)c(Oc4ccc(F)c(C#N)c4)c(F)cc32)cc1]

To a solution of 2-fluoro-5-((6-fluoro-4-(methylthio)-1-tosyl-1H-indol-5-yl)oxy)benzonitrile (Intermediate 23C, 1.1 g, 2.34 mmol) in THF (18 mL) was added acetic acid (6 mL) followed by a solution of hydrogen peroxide (30% in water, 2.66 g, 23.4 mmol). The reaction mixture was stirred at room temperature overnight and then at 40° C. for two days. The mixture was diluted with ethyl acetate (100 mL), washed with water (30 mL×2), saturated sodium bicarbonate solution (20 mL) and brine (20 mL×2), dried over sodium sulfate, filtered and concentrated. The resulting residue was purified by flash silica gel column chromatography (0-50% ethyl acetate in petroleum ether) to give the title compound as a solid (803 mg, 70%). MS: 487 m/z [M+H]⁺.

### Intermediate 19B

### N-((5-(3-Cyano-4-fluorophenoxy)-6-fluoro-1-tosyl-1H-indol-4-yl)(methyl)(oxo)-16-sulfaneylidene)-2,2,2-trifluoroacetamide

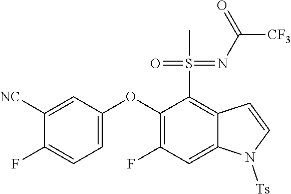 [SMILES: Cc1ccc(S(=O)(=O)n2ccc3c(S(C)(=O)=NC(=O)C(F)(F)F)c(Oc4ccc(F)c(C#N)c4)c(F)cc32)cc1]

To a solution of 2-fluoro-5-((6-fluoro-4-(methylsulfinyl)-1-tosyl-1H-indol-5-yl)oxy)benzonitrile (Intermediate 19A, 833 mg, 1.7 mmol) in dichloromethane (25 mL) were added CF₃CONH₂ (386 mg, 3.42 mmol), MgO (274 mg, 6.84 mmol), PhI(OAc)₄ (1.13 g, 2.57 mmol) and Rh₂(OAc)₄ (76 mg, 171 μmol). The reaction mixture was stirred at room temperature over weekend, filtered and rinsed with dichloromethane (10 mL×2). The filtrate was concentrated and the resulting residue was purified by flash silica gel column chromatography (0-50% ethyl acetate in petroleum ether) to give the title compound as a solid (380 mg, 35%) and recovered starting material 2-fluoro-5-((6-fluoro-4-(methylsulfinyl)-1-tosyl-1H-indol-5-yl)oxy)benzonitrile (380 mg, 46%). MS: 598 m/z [M+H]⁺. ¹H NMR (400 MHz, CDCl₃) δ 8.25 (d, J=10.3 Hz, 1H), 7.88-7.82 (m, 3H), 7.49 (dd, J=3.8, 0.5 Hz, 1H), 7.37 (d, J=8.1 Hz, 2H), 7.18-7.02 (m, 3H), 3.66 (s, 3H), 2.44 (s, 3H) ppm.

### Intermediate 20

### 3-((4-(Hydroxymethyl)-1H-indol-5-yl)oxy)benzonitrile

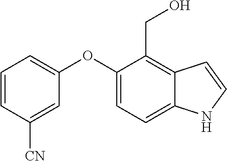 [SMILES: N#Cc1cccc(Oc2ccc3[nH]ccc3c2CO)c1]

To a solution of 3-((4-formyl-1H-indol-5-yl)oxy)benzonitrile (Intermediate 20A, 2.32 g, 8.85 mmol) in methanol (20 mL) was added sodium borohydride (1.01 g, 26.6 mmol). The reaction mixture was stirred at 25° C. for two hours, quenched with saturated ammonium chloride (100 mL) and extracted with ethyl aceate (50 mL×3). The combined organic extracts were washed with brine (50 mL), dried over sodium sulfate, filtered and concentrated. The resulting residue was purified by flash chromatography over silica (petroleum ether:ethyl acetate, v/v, 2/1) to afford the title compound as a light yellow oil (2.16 g, 76% from 2 steps). MS: 263 m/z [M−H]⁻.

### Intermediate 20A

### 3-((4-Formyl-1H-indol-5-yl)oxy)benzonitrile

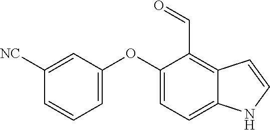 [SMILES: N#Cc1cccc(Oc2ccc3[nH]ccc3c2C=O)c1]

To a solution of 3-((4-vinyl-1H-indol-5-yl)oxy)benzonitrile (Intermediate 14, 2.8 g, 10.8 mmol) in THF (30 mL) and water (10 mL) were added osmium tetroxide (0.5 mL), sodium periodate (6.91 g, 32.3 mmol). The reaction mixture was stirred at 25° C. for two hours, quenched with saturated aqueous sodium thiosulfate (100 mL) and extracted with ethyl aceate (50 mL×3). The combined organic extracts were washed with brine (50 mL), dried over sodium sulfate, filtered and concentrated to give the title compound as brown oil (2.32 g, crude), which was used directly in the next step. MS: 263 m/z [M+H]⁺.

The following intermediate was prepared utilizing the procedures described for Intermediate 20.

| No. | Structure | Name | MS m/z [M + H]⁺ |
|---|---|---|---|
| 20-1 | 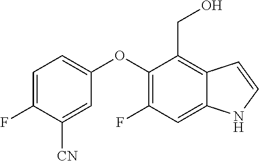 [SMILES: N#Cc1cc(Oc2c(F)cc3[nH]ccc3c2CO)ccc1F] | 2-fluoro-5-((6-fluoro-4-(hydroxymethyl)- 1H-indol-5-yl)oxy)benzonitrile | 301 |

### Intermediate 21

### Methyl 3-(3-(3-bromo-2-oxopropyl)phenyl)propanoate

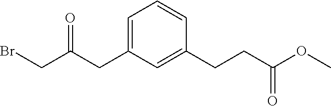 [SMILES: COC(=O)CCc1cccc(CC(=O)CBr)c1]

To a solution of 2-(3-(3-methoxy-3-oxopropyl)phenyl)acetic acid (Intermediate 21B, 1.4 g, 6.3 mmol) in dichloromethane (10 mL) was added oxalyl chloride (2 mL, 25.2 mmol) and a drop 1 drop of DMF. The reaction mixture was stirred at room temperature for three hours and concentrated. To the residue was added 30 mL of heptane and re-evaporate again to remove any residue oxalyl chloride. The resulting residue was dissolved in 1:1 mixture of THF and acetonitrile (10 mL), cooled to 0° C. trimethylsilyl diazomethane (2 M, 12.6 mL, 25.2 mmol) was added dropwise over 10 minutes. After the addition was completed, the mixture was allowed to warm to room temperature overnight. The solvent was evaporated and the residue was dissolved in 5 mL of dichloromethane and cooled to 0° C. A solution of hydrogen bromide in acetic acid (2.3 g, 1.49 g/mL, 28.3 mmol) was added dropwise over 10 minutes (vigorous gas evolution noted), stirred for another 30 minutes and then diluted with brine (50 mL) and ethyl acetate (100 mL). The separated organic phase was dried over magnesium sulfate and concentrated. The resulting residue was purified by silica gel column chromatography (0-30% ethyl acetate in petroleum ether) to give the title compound as an oil (950 mg, 50%). MS: 321, 323 m/z [M+Na]⁺.

### Intermediate 21A

### (E)-2-(3-(3-Methoxy-3-oxoprop-1-en-1-yl)phenyl)acetic acid

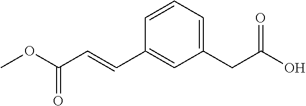 [SMILES: COC(=O)/C=C/c1cccc(CC(=O)O)c1]

To a stirred solution of 3-iodophenylacetic acid (2 g, 7.6 mmol), methyl acrylate (2 g, 22.8 mmol) and triethylamine (3.8 g, 38.0 mmol) in DMF (50 mL) was added tri(o-tolyl)phosphine (456 mg, 1.5 mmol), followed by palladium(II) acetate (170 mg, 0.76 mmol). The reaction mixture was heated at 110° C. overnight in a sealed tube, cooled to room temperature, diluted with ethyl acetate (100 mL) and washed with water (100 mL×2) and brine, dried over sodium sulfate and concentrated. The resulting residue was purified by flash silica gel column chromatography (0-40% ethyl acetate in petroleum ether) to give the title compound as a yellow oil (1.4 g, 80%). MS: 221 m/z [M+Na]⁺.

### Intermediate 21B

### 2-(3-(3-Methoxy-3-oxopropyl)phenyl)acetic acid

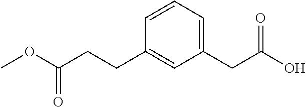 [SMILES: COC(=O)CCc1cccc(CC(=O)O)c1]

A suspension of (E)-2-(3-(3-methoxy-3-oxoprop-1-en-1-yl)phenyl)acetic acid (Intermediate 21A, 1.4 g, 6.3 mmol), Pd/C (140 mg, 5%) and THF (10 mL) in a autoclave was placed under a hydrogen balloon and stirred for sixteen hours at room temperature. The catalyst was removed by filtration and washed with THF (10 mL). The filtrate was concentrated to give the title compound as a yellow oil (1.4 g, 99%). MS: 240 m/z [M+H₂O]⁺.

### Intermediate 21-1

### Ethyl 3-(3-(3-(2-bromoacetyl)tetrahydrofuran-3-yl)phenyl)propanoate

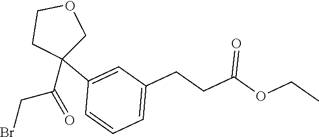 [SMILES: CCOC(=O)CCc1cccc(C2(C(=O)CBr)CCOC2)c1]

To a solution of 3-(3-(3-ethoxy-3-oxopropyl)phenyl)tetrahydrofuran-3-carboxylic acid (Intermediate 21-1D, 200 mg, 0.68 mmol) in dichloromethane (4 mL) were added oxalyl chloride (270 mg, 2.05 mmol) and DMF (2 drops) at 0° C. The reaction mixture was stirred at room temperature for one hour and concentrated. To the residue was added 30 mL of heptane and evaporated again to remove any residual oxalyl chloride. The resulting product was dissolved in 1:1 mixture of THF and acetonitrile (2 mL), cooled to 0° C. and a solution of trimethylsilyldiazomethane (2N, 1.4 mL, 2.74 mmol) in hexane was added dropwise over five minutes. After the addition was complete, the mixture was allowed to warm to room temperature and stirred for two hours. The yellow solution was then cooled to 0° C., a 2N solution of hydrogen bromide in acetic acid (223 mg, 2.74 mmol, 1.37 mL) was added (vigorous gas evolution noted) (4 N hydrochloric acid in 1,4-dioxane was used when chloroketone instead of bromoketone was desired), stirred for one hour and diluted with brine (20 mL) and ethyl acetate (50 mL). The organic phase was dried over sodium sulfate, filtered and concentrated. The resulting residue was purified by flash chromatography over silica (petroleum ether/ethyl acetate, v/v, 5/1) to give the title compound as a light yellow oil (170 mg, 66%). MS: 369, 371 m/z [M+H]⁺.

### Intermediate 21-1A

### 3-(3-Bromophenyl)tetrahydrofuran-3-carbonitrile

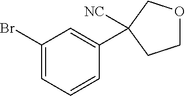 [SMILES: N#CC1(c2cccc(Br)c2)CCOC1]

To a stirred solution of 2-(3-bromophenyl)acetonitrile (2 g, 10.3 mmol) and 1-chloro-2-(chloromethoxy)ethane (1.58 g, 12.3 mmol) in DMF (15 mL) was added sodium hydride (0.74 g, 30.8 mmol) at 0° C. The reaction mixture was stirred at 25° C. overnight, diluted with ethyl acetate (50 mL) washed with water (40 mL×3) and brine (30 mL×2), dried over sodium sulfate and concentrated. The resulting residue was purified by flash chromatography over silica (petroleum/ethyl acetate, v/v, 5/1) to afford the title compound as a brown liquid (1.15 g, 45%). MS: 252, 254 m/z [M+H]⁺.

### Intermediate 21-1B

### 3-(3-Bromophenyl)tetrahydrofuran-3-carboxylic acid

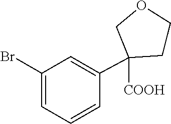 [SMILES: O=C(O)C1(c2cccc(Br)c2)CCOC1]

To a stirred solution of 3-(3-bromophenyl)tetrahydrofuran-3-carbonitrile (Intermediate 21-1A, 1.15 g, 4.6 mmol) in ethylene glycol (10 mL) and water (5 mL) was added potassium hydroxide (2.58 g, 46 mmol). The reaction mixture was stirred at 120° C. overnight, cooled to room temperature, acidified to pH˜7 with hydrochloric acid (1N) and extracted with ethyl acetate (50 mL×3). The combined organic phase was washed with brine (100 mL×3), dried and concentrated. The resulting residue was purified by flash chromatography over silica (petroleum/ethyl acetate, v/v, 2/1) to afford the title compound as a white solid (1.07 g, 86%). MS: 271, 273 m/z [M+H]⁺.

### Intermediate 21-1C

### (E)-3-(3-(3-Ethoxy-3-oxoprop-1-en-1-yl)phenyl)tetrahydrofuran-3-carboxylic acid

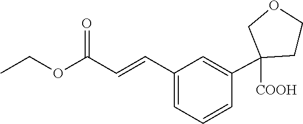 [SMILES: CCOC(=O)/C=C/c1cccc(C2(C(=O)O)CCOC2)c1]

To a solution of 3-(3-bromophenyl)tetrahydrofuran-3-carboxylic acid (500 mg, 2 mmol) and ethyl acrylate (260 mg, 2.6 mmol) in DMF (5 mL) were added triethylamine (605 mg, 6 mmol), tri(o-tolyl)phosphine (120 mg, 0.3 mmol) and Pd(OAc)₂ (70 mg, 0.3 mmol). The reaction mixture was stirred at 100° C. for four hours under nitrogen atmosphere in a sealed tube, cooled to room temperature, diluted with ethyl acetate (40 mL), washed with brine (10 mL×2), dried and concentrated. The resulting residue was purified by flash chromatography over silica (petroleum ether/ethyl acetate, v/v, 5/1) to afford the title compound (305 mg, 56%). MS: 291 m/z [M+H]⁺.

### Intermediate 21-((2

### 3-(3-(3-Ethoxy-3-oxopropyl)phenyl)tetrahydrofuran-3-carboxylic acid

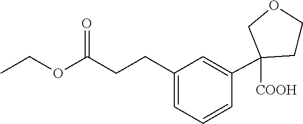 [SMILES: CCOC(=O)CCc1cccc(C2(C(=O)O)CCOC2)c1]

To a stirred solution of 3-(3-(3-ethoxy-3-oxoprop-1-en-1-yl)phenyl)tetrahydrofuran-3-carboxylic acid (Intermediate 21-1C, 305 mg, 1.05 mmol) in ethanol (5 mL) was added Pd/C (60 mg, 10%). The reaction mixture was stirred at room temperature under hydrogen balloon for three hours, filtered and washed with ethanol (5 mL×3). The filtrate was concentrated and the resulting residue was purified by flash chromatography over silica (petroleum/ethyl acetate, v/v, 2/1) to afford the title compound (240 mg, 79%) as a liquid. MS: 293 m/z [M+H]⁺. ¹H NMR (400 MHz, CDCl₃) δ 7.23-7.33 (m, 1H), 7.10-7.19 (m, 3H), 4.72 (d, J=8.4 Hz, 1H), 4.12 (q, J=7.2 Hz, 2H), 3.92-4.04 (m, 2H), 3.86 (d, J=8.4 Hz, 1H), 2.89-3.06 (m, 3H), 2.61 (t, J=7.6 Hz, 2H), 2.14-2.32 (m, 1H), 1.23 (t, J=7.2 Hz, 3H) ppm.

The following intermediates were prepared utilizing the procedures described for Intermediates 21 and/or 21-1.

| | | | | MS m/z |
|---|---|---|---|---|
| No. | Structure | Name | ¹H NMR | [M + H]⁺ |
| 21-2 | 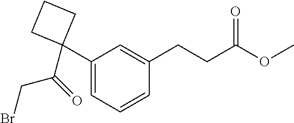 [SMILES: COC(=O)CCc1cccc(C2(C(=O)CBr)CCC2)c1] | methyl 3-(3-(1-(2- bromoacetyl)cyclobutyl) phenyl)propanoate | | 339/341 |
| 21-3 | 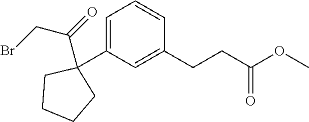 [SMILES: COC(=O)CCc1cccc(C2(C(=O)CBr)CCCC2)c1] | methyl 3-(3-(1-(2- bromoacetyl)cyclopentyl) phenyl)propanoate | | 353/355 |
| 21-4 | 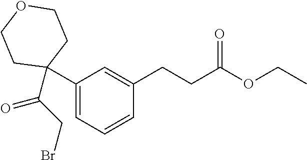 [SMILES: CCOC(=O)CCc1cccc(C2(C(=O)CBr)CCOCC2)c1] | ethyl 3-(3-(4-(2- bromoacetyl)tetrahydro- 2H-pyran-4- yl)phenyl)propanoate | | 383/385 |
| 21-5 | 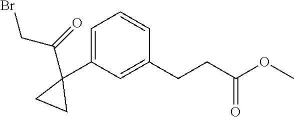 [SMILES: COC(=O)CCc1cccc(C2(C(=O)CBr)CC2)c1] | methyl 3-(3-(1-(2- bromoacetyl)cyclopropyl) phenyl)propanoate | | 325/327 |
| 21-6 | 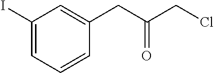 [SMILES: O=C(CCl)Cc1cccc(I)c1] | 1-chloro-3-(3- iodophenyl)propan-2-one | (400 MHz, CDCl₃) δ 7.67-7.56 (m, 2H), 7.20 (d, J = 7.7 Hz, 1H), 7.08 (t, J = 7.7 Hz, 1H), 4.12 (s, 2H), 3.85 (s, 2H) ppm. | |
| 21-7 | 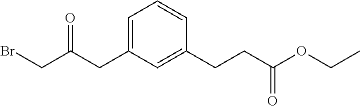 [SMILES: CCOC(=O)CCc1cccc(CC(=O)CBr)c1] | ethyl 3-(3-(3-bromo-2- oxopropyl)phenyl) propanoate | | 313/315 |
| 21-8 | 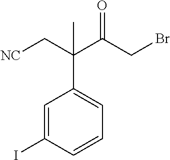 [SMILES: CC(CC#N)(C(=O)CBr)c1cccc(I)c1] | 5-bromo-3-(3- iodophenyl)-3-methyl-4- oxopentanenitrile | (400 MHz, CDCl₃) δ 7.75 (d, J = 7.2 Hz, 1H), 7.61 (s, 1H), 7.23- 7.17 (m, 2H), 3.93 (d, J = 14.0 Hz, 1H), 3.76 (d, J = 13.6 Hz, 1H), 2.96 (d, J = 16.8 Hz, 1H), 2.85 (d, J = 17.2 Hz, 1H), 1.85 (s, 3H) ppm | |
| 21-9 | 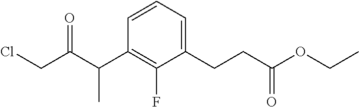 [SMILES: CCOC(=O)CCc1cccc(C(C)C(=O)CCl)c1F] | ethyl 3-(3-(4-chloro-3- oxobutan-2-yl)-2- fluorophenyl)propanoate | (400 MHz, CDCl₃) δ 7.26-7.04 (m, 3H), 4.29 (q, J = 6.8 Hz, 1H), 4.11 (q, J = 7.2 Hz, 2H), 4.13-4.09 (m, 2H), 2.99 (t, J = 7.2 Hz, 2H), 2.63 (t, J = 8.0 Hz, 2H), 1.45 (d, J = 7.2 Hz, 3H), 1.23 (t, J = 7.2 Hz, 3H) ppm | 301 |
| 21-10 | 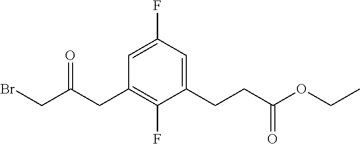 [SMILES: CCOC(=O)CCc1cc(F)cc(CC(=O)CBr)c1F] | ethyl 3-(3-(3-bromo-2- oxopropyl)-2,5- difluorophenyl)propanoate | | 349, 351 |
| 21-11 | 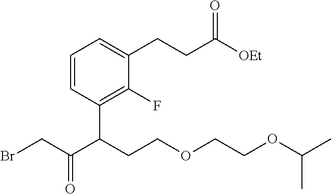 [SMILES: CCOC(=O)CCc1cccc(C(CCOCCOC(C)C)C(=O)CBr)c1F] | ethyl 3-(3-(1-bromo-5-(2- isopropoxyethoxy)-2- oxopentan-3-yl)-2- fluorophenyl)propanoate | | 483, 485 m/z [M + Na]⁺ |
| 21-12 | 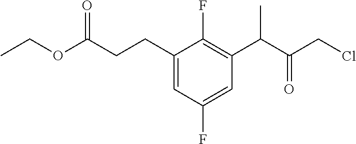 [SMILES: CCOC(=O)CCc1cc(F)cc(C(C)C(=O)CCl)c1F] | ethyl 3-(3-(4-chloro-3- oxobutan-2-yl)-2,5- difluorophenyl)propanoate | | 319 |
| 21-13 | 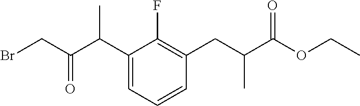 [SMILES: CCOC(=O)C(C)Cc1cccc(C(C)C(=O)CBr)c1F] | ethyl 3-(3-(4-bromo-3- oxobutan-2-yl)-2- fluorophenyl)-2- methylpropanoate | | 381, 383 m/z [M + Na]⁺ |
| 21-14 | 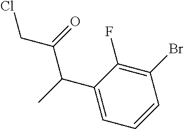 | 3-(3-bromo-2- fluorophenyl)-1- chlorobutan-2-one | | 279, 281 |

### Intermediate 22

### 1,2-Difluoro-3,4-dimethyl-5-nitrobenzene

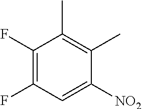

A mixture of 3-bromo-1,2-difluoro-4-methyl-5-nitrobenzene (Intermediate 6A, 10 g, 40 mmol), methylboronic acid (12 g, 200 mmol), Pd(dppf)Cl₂ (2.9 mg, 4.0 mmol) and sodium bicarbonate (10 g, 120 mmol) in 1,4-dioxane (160 mL) and water (40 mL) was stirred under nitrogen atmosphere at 80° C. for 3 days. The mixture was filtered and washed with ethyl acetate (300 mL). The organic phase was separated, washed with water (100 mL×2) and brine (50 mL), dried over sodium sulfate, filtered and concentrated. The resulting residue was purified by flash chromatography over silica (petroleum ether:ethyl acetate, v/v, 12/1) to afford the title compound as a light yellow oil (6.4 g, 85%). ¹H NMR (400 MHz, CDCl₃) δ 7.59 (t, J=8.4 Hz, 1H), 2.42 (s, 3H), 2.32 (d, J=2.4 Hz, 3H) ppm.

### Intermediate 23

### 2-Fluoro-5-((6-fluoro-4-(methylthio)-1H-indol-5-yl)oxy)benzonitrile

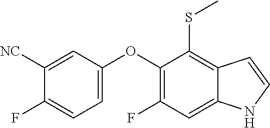 [SMILES: CSc1c(Oc2ccc(F)c(C#N)c2)c(F)cc2[nH]ccc12]

To a 1M solution of tetrabutylammonium fluoride in THF (106 mL) was added 2-fluoro-5-((6-fluoro-4-(methylthio)-1-tosyl-1H-indol-5-yl)oxy)benzonitrile (Intermediate 23C, 10 g, 21.2 mmol). The solution was stirred at 75° C. for two hours, cooled to room temperature, water (400 mL) was added and extracted with ethyl acetate (300 mL×2). The combined organic extracts were washed with brine (400 mL), dried over sodium sulfate, filtered and concentrated to give an oil. The oil was purified by flash chromatography over silica (Petroleum ether/ethyl acetate, v/v, 100/1 to 1/1) to give the title compound as a white solid (3.65 g, 47%). MS: 317 m/z [M+H]⁺. ¹H NMR: (400 MHz CDCl₃) δ 8.30 (s, 1H), 7.23-7.25 (m, 1H), 7.06-7.16 (m, 3H), 6.94-6.95 (m, 1H), 6.70-6.71 (m, 1H), 2.39 (s, 3H) ppm.

### Intermediate 23A

### 2-Fluoro-5-((6-fluoro-4-((4-methoxybenzyl)thio)-1-tosyl-1H-indol-5-yl)oxy)benzonitrile

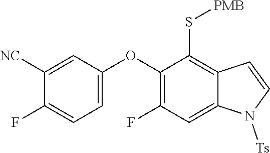

To a solution of 5-((4-bromo-6-fluoro-1-tosyl-1H-indol-5-yl)oxy)-2-fluorobenzonitrile (Intermediate 7, 57 μg, 11.3 mmol) in dioxane (1000 mL) were added (4-methoxyphenyl)methanethiol (17.5 g, 113 mmol, 16 mL), diisopropylethylamine (29.3 g, 226 mmol, 40 mL), Xantphos (6.6 g, 11.3 mmol) and Pd₂(dba)₃ (5.2 g, 5.7 mmol) under nitrogen atmosphere. The suspension was degassed, purged with nitrogen for 3 times, stirred under nitrogen at 100° C. for twelve hours, quenched with water (200 mL) and extracted with ethyl acetate (200 ml×2). The combined organic extracts were washed with brine (300 mL), dried over sodium sulfate, filtered and concentrated to give a solid. The solid was purified by flash chromatography over silica (petroleum ether/ethyl acetate, v/v, 50/1 to 1/1) to afford the title compound as a yellow solid (104 g, 80%). ¹H NMR: (400 MHz CDCl₃) δ 7.76-7.84 (m, 3H), 7.58-7.59 (m, 1H), 7.31 (d, J=8.4 Hz, 2H), 7.06-7.08 (m, 2H), 6.89-6.91 (m, 3H), 7.75 (d, J=3.2 Hz, 1H), 6.62 (d, J=8.8 Hz, 2H), 3.96 (s, 2H), 3.74 (s, 3H), 2.40 (s, 3H) ppm.

### Intermediate 23B

### 2-Fluoro-5-((6-fluoro-4-mercapto-1-tosyl-1H-indol-5-yl)oxy)benzonitrile

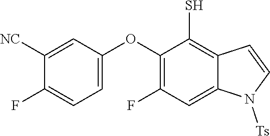 [SMILES: Cc1ccc(S(=O)(=O)n2ccc3c(S)c(Oc4ccc(F)c(C#N)c4)c(F)cc32)cc1]

Trifluoroacetic acid (250 mL) was added dropwise to a solution of 2-fluoro-5-((6-fluoro-4-((4-methoxybenzyl)thio)-1-tosyl-1H-indol-5-yl)oxy)benzonitrile (Intermediate 23A, 54 g, 94 mmnol) in anisole (250 mL). The reaction mixture was stirred at 50° C. for twelve hours and concentrated to afford the title compound as a brown solid (54 g, crude). MS: 457 m/z [M+H]⁺.

### Intermediate 23C

### 2-Fluoro-5-((6-fluoro-4-(methylthio)-1-tosyl-1H-indol-5-yl)oxy)benzonitrile

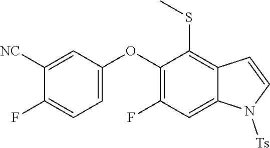 [SMILES: CSc1c(Oc2ccc(F)c(C#N)c2)c(F)cc2c1ccn2S(=O)(=O)c1ccc(C)cc1]

To a solution of 2-fluoro-5-((6-fluoro-4-mercapto-1-tosyl-1H-indol-5-yl)oxy)benzonitrile (Intermediate 23B, 54 g, 118 mmol) in THF (250 mL) was added PPh₃ (27 g, 103 mmol). The solution was stirred at 25° C. for four hours and concentrated. The residue was dissolved in acetone (400 mL) and potassium carbonate (32.7 g, 237 mmol) and methyl iodide (19.3 g, 136 mmol, 8.5 mL) were added, stirred at 25° C. for six hours, filtered and concentrated. The resulting residue was purified by flash chromatography over silica to afford the title compound as a yellow solid (44 g. 79%). MS: 471 m/z [M+H]⁺.

The following intermediates were prepared utilizing similar procedures described for Intermediate 23.

| | | | MS m/z |
|---|---|---|---|
| No. | Structure | Name | [M + H]⁺ |
| 23-1 | 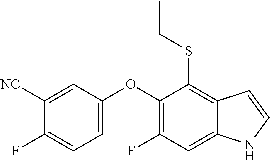 [SMILES: CCSc1c(Oc2ccc(F)c(C#N)c2)c(F)cc2[nH]ccc12] | 5-((4-(ethylthio)-6-fluoro- 1H-indol-5-yl)oxy)-2- fluorobenzonitrile | 331 |
| 23-2 | 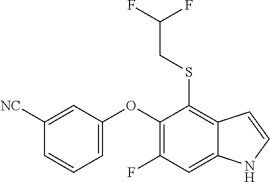 [SMILES: N#Cc1cccc(Oc2c(F)cc3[nH]ccc3c2SCC(F)F)c1] | 3-((4-((2,2- difluoroethyl)thio)-6-fluoro- 1H-indol-5- yl)oxy)benzonitrile | 349 |
| 23-3 | 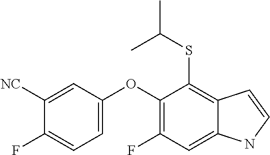 [SMILES: CC(C)Sc1c(Oc2ccc(F)c(C#N)c2)c(F)cc2[nH]ccc12] | 2-fluoro-5-((6-fluoro-4- (isopropylthio)-1H-indol-5- yl)oxy)benzonitrile | 345 |
| 23-4 | 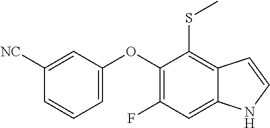 [SMILES: CSc1c(Oc2cccc(C#N)c2)c(F)cc2[nH]ccc12] | 3-((6-fluoro-4-(methylthio)- 1H-indol-5- yl)oxy)benzonitrile | 299 |
| 23-5 | 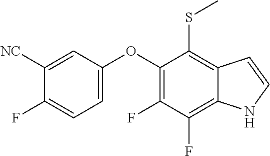 [SMILES: CSc1c(Oc2ccc(F)c(C#N)c2)c(F)c(F)c2[nH]ccc12] | 5-((6,7-difluoro-4- (methylthio)-1H-indol-5- yl)oxy)-2-fluorobenzonitrile | 335 |
| 23C-1 | 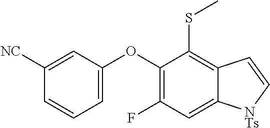 [SMILES: CSc1c(Oc2cccc(C#N)c2)c(F)cc2c1ccn2S(=O)(=O)c1ccc(C)cc1] | 3-((6-fluoro-4-(methylthio)- 1-tosyl-1H-indol-5- yl)oxy)benzonitrile | 453 |

### Intermediate 24

### 2-Fluoro-5-((6-fluoro-2H-pyrrolo[3,2-b]pyridin-5-yl)oxy)benzimidamide

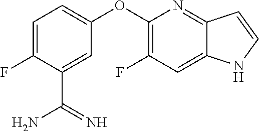 [SMILES: N=C(N)c1cc(Oc2nc3cc[nH]c3cc2F)ccc1F]

To a solution of 2-fluoro-5-((6-fluoro-1H-pyrrolo[3,2-b]pyridin-5-yl)oxy)benzonitrile (Intermediate 17, 380 mg, 1.12 mmol) in THF (4 mL) was added a 1M solution of lithium bis(trimethylsilyl)amide in THF (9 mL, 9 mmol). The reaction mixture was heated at 25° C. for 18 hours, quenched with water (20 mL) and extracted with dichloromethane (30 mL×4). The combined organic phase was washed with brine (20 mL), dried over magnesium sulfate and concentrated to afford the title compound as white solid (350 mg, 74%). MS: 289 m/z [M+H]⁺.

The following intermediates were prepared utilizing similar procedures described for Intermediate 24.

| | | | MS m/z |
|---|---|---|---|
| No. | Structure | Name | [M + H]⁺ |
| 24-1 | 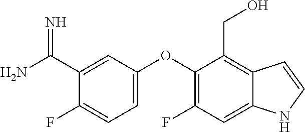 [SMILES: N=C(N)c1cc(Oc2c(F)cc3[nH]ccc3c2CO)ccc1F] | 2-fluoro-5-((6-fluoro-4-(hydroxymethyl)- 1H-indol-5-yl)oxy)benzimidamide | 318 |
| 24-2 | 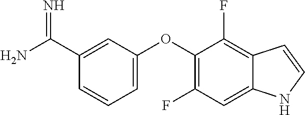 [SMILES: N=C(N)c1cccc(Oc2c(F)cc3[nH]ccc3c2F)c1] | 3-((4,6-difluoro-1H-indol-5- yl)oxy)benzimidamide | 288 |
| 24-3 | 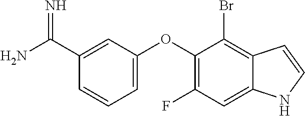 [SMILES: N=C(N)c1cccc(Oc2c(F)cc3[nH]ccc3c2Br)c1] | 3-((4-bromo-6-fluoro-1H-indol-5- yl)oxy)benzimidamide | 348/350 |
| 24-4 | 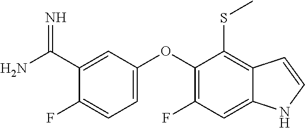 [SMILES: CSc1c(Oc2ccc(F)c(C(=N)N)c2)c(F)cc2[nH]ccc12] | 2-fluoro-5-((6-fluoro-4-(methylthio)-1H- indol-5-yl)oxy)benzimidamide | 334 |
| 24-5 | 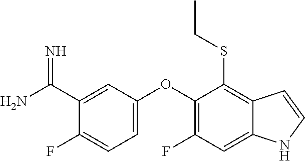 [SMILES: CCSc1c(Oc2ccc(F)c(C(=N)N)c2)c(F)cc2[nH]ccc12] | 5-((4-(ethylthio)-6-fluoro-1H-indol-5- yl)oxy)-2-fluorobenzimidamide | 348/350 |
| 24-6 | 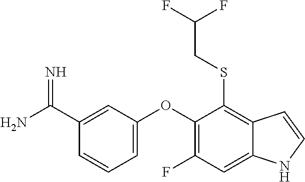 [SMILES: N=C(N)c1cccc(Oc2c(F)cc3[nH]ccc3c2SCC(F)F)c1] | 3-((4-((2,2-difluoroethyl)thio)-6-fluoro-1H- indol-5-yl)oxy)benzimidamide | 366 |
| 24-7 | 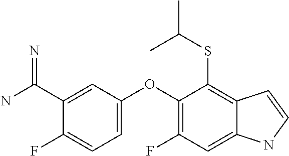 [SMILES: CC(C)Sc1c(Oc2ccc(F)c(C(=N)N)c2)c(F)cc2[nH]ccc12] | 2-fluoro-5-((6-fluoro-4-(isopropylthio)-1H- indol-5-yl)oxy)benzimidamide | 362 |
| 24-8 | 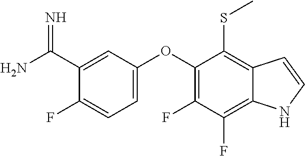 [SMILES: CSc1c(Oc2ccc(F)c(C(=N)N)c2)c(F)c(F)c2[nH]ccc12] | 5-((6,7-difluoro-4-(methylthio)-1H-indol-5- yl)oxy)-2-fluorobenzimidamide | 352 |
| 24-9 | 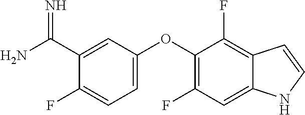 [SMILES: N=C(N)c1cc(Oc2c(F)cc3[nH]ccc3c2F)ccc1F] | 5-((4,6-difluoro-1H-indol-5-yl)oxy)-2- fluorobenzimidamide | 306 |
| 24-10 | 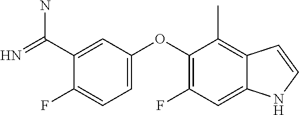 [SMILES: Cc1c(Oc2ccc(F)c(C(=N)N)c2)c(F)cc2[nH]ccc12] | 2-fluoro-5-((6-fluoro-4-methyl-1H-indol-5- yl)oxy)benzimidamide | 302 |
| 24-11 | 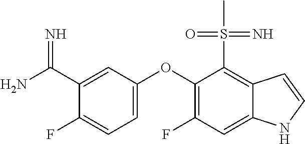 [SMILES: CS(=N)(=O)c1c(Oc2ccc(F)c(C(=N)N)c2)c(F)cc2[nH]ccc12] | 2-fluoro-5-((6-fluoro-4-(S- methylsulfonimidoyl)-1H-indol-5- yl)oxy)benzimidamide | 365 |
| 24-12 | 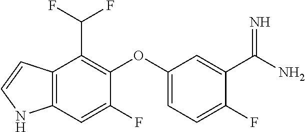 [SMILES: N=C(N)c1cc(Oc2c(F)cc3[nH]ccc3c2C(F)F)ccc1F] | 5-((4-(difluoromethyl)-6-fluoro-1H-indol-5- yl)oxy)-2-fluorobenzimidamide | 338 |
| 24-13 | 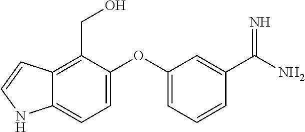 [SMILES: N=C(N)c1cccc(Oc2ccc3[nH]ccc3c2CO)c1] | 3-((4-(hydroxymethyl)-1H-indol-5- yl)oxy)benzimidamide | 282 |
| 24-14 | 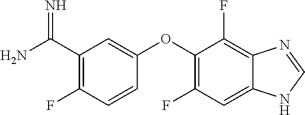 [SMILES: N=C(N)c1cc(Oc2c(F)cc3[nH]cnc3c2F)ccc1F] | 5-((4,6-difluoro-1H-benzo[d]imidazol-5- yl)oxy)-2-fluorobenzimidamide | 307 |
| 24-15 | 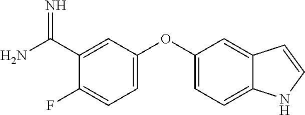 [SMILES: N=C(N)c1cc(Oc2ccc3[nH]ccc3c2)ccc1F] | 2-fluoro-5-(1H-indole-5- carbonyl)benzimidamide | 282 |
| 24-16 | 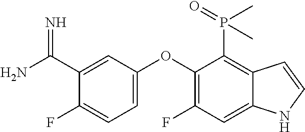 [SMILES: CP(C)(=O)c1c(Oc2ccc(F)c(C(=N)N)c2)c(F)cc2[nH]ccc12] | 5-((4-(Dimethylphosphoryl)-6-fluoro-1H- indol-5-yl)oxy)-2-fluorobenzimidamide | 364 |
| 24-17 | 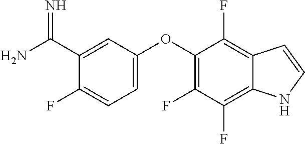 [SMILES: N=C(N)c1cc(Oc2c(F)c(F)c3[nH]ccc3c2F)ccc1F] | 2-Fluoro-5-((4,6,7-trifluoro-1H-indol-5- yl)oxy)benzimidamide | 324 |
| 24-18 | 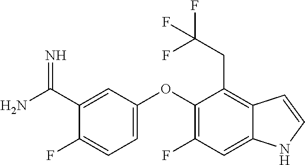 [SMILES: N=C(N)c1cc(Oc2c(F)cc3[nH]ccc3c2CC(F)(F)F)ccc1F] | 2-fluoro-5-((6-fluoro-4-(2,2,2- trifluoroethyl)-1H-indol-5- yl)oxy)benzimidamide | 370 |
| 24-19 | 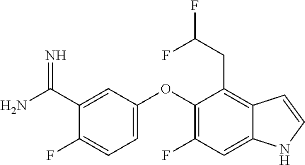 [SMILES: N=C(N)c1cc(Oc2c(F)cc3[nH]ccc3c2CC(F)F)ccc1F] | 5-((4-(2,2-difluoroethyl)-6-fluoro-1H-indol- 5-yl)oxy)-2-fluorobenzimidamide | 352 |
| 24-20 | 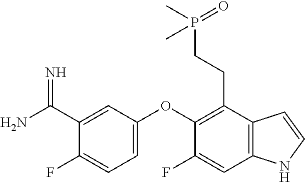 [SMILES: CP(C)(=O)CCc1c(Oc2ccc(F)c(C(=N)N)c2)c(F)cc2[nH]ccc12] | 5-((4-(2-(dimethylphosphoryl)ethyl)-6- fluoro-1H-indol-5-yl)oxy)-2- fluorobenzimidamide | 392 |
| 24-21 | 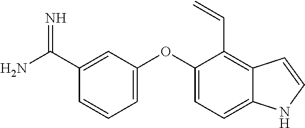 [SMILES: C=Cc1c(Oc2cccc(C(=N)N)c2)ccc2[nH]ccc12] | 3-((4-vinyl-1H-indol-5- yl)oxy)benzimidamide | 278 |

### Intermediate 25

### 2-(3-Iodophenyl)acetohydrazide

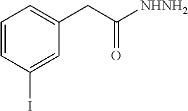 [SMILES: NNC(=O)Cc1cccc(I)c1]

A mixture of methyl 2-(3-iodophenyl)acetate (10 g, 36.22 mmol) and hydrazine hydrate (4.55 mL, 144.89 mmol) in ethanol (30 mL) was heated at 70° C. overnight. The solvent was evaporated and the residue was triturated with methanol to give the first crop of desired product (7.5 g). The methanol solution was then evaporated and the solid was purified by flash chromatography over silica (0-10% methanol in dichloromethane containing 0.5% concentrate ammonia) to give another 1.5 g of product. 2-(3-iodophenyl)acetohydrazide was obtained as a white solid (9 g, 90%). MS m/z: 277 m/z [M+H]⁺.

The following intermediate was prepared utilizing the procedures described for Intermediate 25.

| No. | Structure | Name | MS m/z [M + H]⁺ |
|---|---|---|---|
| 25-1 | 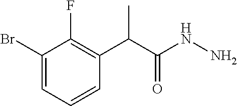 | 2-(3-bromo-2- fluorophenyl)propanehydrazide | 261, 263 |

### Intermediate 26

### Methyl 3-(3-(3-formyl-2-methyloxiran-2-yl)phenyl)propanoate

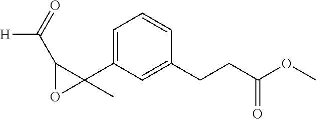 [SMILES: COC(=O)CCc1cccc(C2(C)OC2C=O)c1]

To a solution of methyl 3-(3-(3-(hydroxymethyl)-2-methyloxiran-2-yl)phenyl)propanoate (Intermediate 26D, 1.11 g, 4.44 mmol) in dichloromethane (50 mL) were added bis-acetoxy iodobenzene (1.72 g, 5.32 mmol) and TEMPO (69 mg, 0.44 mmol) in one portion. The reaction mixture was stirred at 25° C. for two hours, concentrated to dryness under reduced pressure at room temperature. The resulting residue was purified by flash chromatography over silica (petroleum ether/ethyl acetate, v/v, 5/1) to afford the title compound as a colorless oil (775 mg, 70%). MS: 249 m/z [M+H]⁺. ¹H NMR (400 MHz, CDCl₃) δ 9.58 (d, J=4.8 Hz, 1H), 7.63-7.60 (m, 1H), 7.28-7.14 (m, 3H), 3.32 (d, J=4.8 Hz, 1H), 3.60 (s, 3H), 2.97 (t, J=7.2 Hz, 2H), 2.65 (t, J=7.2 Hz, 2H), 1.84 (s, 3H) ppm.

### Intermediate 26A

### (Z)-3-(4,4,5,5-Tetramethyl-1,3,2-dioxaborolan-2-yl)but-2-en-1-yl acetate

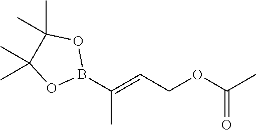 [SMILES: CC(=O)OC/C=C(\C)B1OC(C)(C)C(C)(C)O1]

To a solution of but-2-yn-1-yl acetate (20 g, 179 mmol), B₂(pin)₂ (54 g, 213 mmol), potassium carbonate (4.9 g, 36 mmol) in ether (200 mL) were added CuCl (881 mg, 8.9 mmol), P((C₆H₄OMe))₃ (3.8 g, 11 mmol) and isopropanol (21.4 g, 357 mmol) at 0° C. The reaction mixture was stirred overnight and filtered to remove solid. The filtrate was diluted with ether (100 mL), washed with water, dried with sodium sulfate and concentrated. The resulting residue was purified by flash chromatography over silica to give the title compound as a colorless oil (32 g, 75%). ¹H NMR (400 MHz, CDCl₃) δ 6.32-6.35 (m, 1H), 4.72 (d, J=6.0 Hz, 2H), 2.07 (s, 3H), 1.74 (s, 3H), 1.27 (s, 12H) ppm.

### Intermediate 26B

### Methyl (E)-3-(3-(4-acetoxybut-2-en-2-yl)phenyl)propanoate

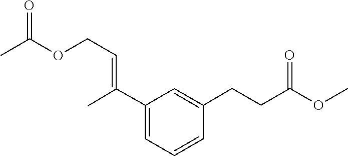 [SMILES: COC(=O)CCc1cccc(/C(C)=C/COC(C)=O)c1]

To a mixture of Pd(dppf)Cl₂ (366 mg, 0.49 mmol) and sodium bicarbonate (4.15 g, 49.4 mmol) in 1,4-dioxane (100 mL) and water (66 mL) was added (Z)-3-(4,4,5,5-tetramethyl-1,3,2-dioxaborolan-2-yl)but-2-en-1-yl acetate (Intermediate 26A, 4.74 g, 19.75 mmol) followed by methyl 3-(3-bromophenyl)propanoate (2.4 g, 9.88 mmol). The resulting mixture was degassed with N₂, heated at 80° C. for three hours, cooled to room temperature and extracted with ethyl acetate (100 mL×2). The combined organic extracts were washed with brine, dried over sodium sulfate, filtered and concentrated. The resulting residue was purified by flash silica gel column chromatography (petroleum ether/ether, v/v, 8/1-2/1) to afford the title compound as a colorless oil (1.8 g, 62%). MS: 277 m/z [M+H]⁺.

### Intermediate 26C

### Methyl (E)-3-(3-(4-hydroxybut-2-en-2-yl)phenyl)propanoate

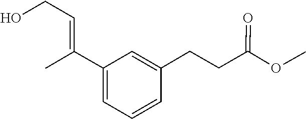 [SMILES: COC(=O)CCc1cccc(/C(C)=C/CO)c1]

Potassium carbonate (0.69 g, 5 mmol) was added to a solution of methyl (E)-3-(3-(4-acetoxybut-2-en-2-yl)phenyl)propanoate (Intermediate 26B, 1.4 g, 5.0 mmol) in methanol (50 mL) and stirred for two hours at room temperature. The solvent was evaporated, the residue was partitioned between water and dichloromethane, and the aqueous layer was further extracted with dichloromethane. The combined organic extracts were washed successively with brine, dried over magnesium sulfate and evaporated to dryness to give the title compound as white solid (1.06 g, 91%). MS m/z: 235 m/z [M+H]⁺.

### Intermediate 26D

### Methyl 3-(3-(3-(hydroxymethyl)-2-methyloxiran-2-yl)phenyl)propanoate

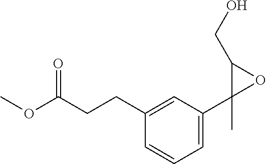 [SMILES: COC(=O)CCc1cccc(C2(C)OC2CO)c1]

A mixture of methyl (E)-3-(3-(4-hydroxybut-2-en-2-yl)phenyl)propanoate (Intermediate 26C, 1.7 g, 7.26 mmol) and 3-chloro-benzenecarboperoxoic acid (70% purity, 2.34 g, 10.16 mmol) in dichloromethane (50 mL) was stirred at room temperature for four hours. The reaction mixture was treated with saturated sodium carbonate solution (30 mL) and extracted with dichloromethane (50 mL×3). The combined organic extracts were washed with brine (30 mL) and concentrated to dryness. The resulting residue was purified by flash chromatography over silica (petroleum ether/ethyl acetate, v/v, 5/1) to give the title compound as a yellow oil (1.11 g, 61%). MS: 251 m/z [M+H]⁺.

The following intermediates were prepared utilizing similar procedures described for Intermediate 26.

| | | | | MS m/z |
|---|---|---|---|---|
| No | Structure | Name | ¹H NMR | [M + H]⁺ |
| 26-1 | 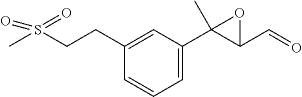 [SMILES: CC1(c2cccc(CCS(C)(=O)=O)c2)OC1C=O] | 3-methyl-3-(3-(2- (methylsulfonyl)ethyl) phenyl)oxirane-2- carbaldehyde | | 286 m/z [M + NH₄⁺] |
| 26-2 | 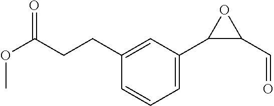 [SMILES: COC(=O)CCc1cccc(C2OC2C=O)c1] | methyl 3-(3-(3- formyloxiran-2- yl)phenyl)propanoate | (400 MHz, CDCl₃) δ 9.48 (d, J = 4.8 Hz, 1H), 7.23-7.19 (m, 3H), 7.14-7.11 (m, 1H), 3.45 (m, 1H), 3.22 (m, 1H), 3.60 (s, 3H), 2.93 (t, J = 7.2 Hz, 2H), 2.63 (t, J = 7.2 Hz, 2H) ppm. | |
| 26-3 | 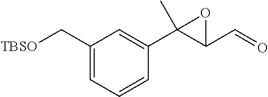 [SMILES: CC1(c2cccc(CO[Si](C)(C)C(C)(C)C)c2)OC1C=O] | 3-(3-(((tert- butyldimethylsilyl)oxy) methyl)phenyl)-3- methyloxirane-2- carbaldehyde | (400 MHz, CDCl₃) δ 9.48 (d, J = 4.8 Hz, 1H), 7.23-7.19 (m, 3H), 7.14-7.11 (m, 1H), 4.64 (s, 2H), 3.22 (d, J = 4.8 Hz, 1H), 1.73 (s, 3H), 0.84 (s, 9H), 0.01 (s, 6H) ppm. | 307 |
| 26-4 | 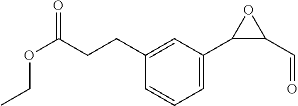 [SMILES: CCOC(=O)CCc1cccc(C2OC2C=O)c1] | ethyl 3-(3-(3- formyloxiran-2- yl)phenyl)propanoate | | 249 |
| 26-5 | 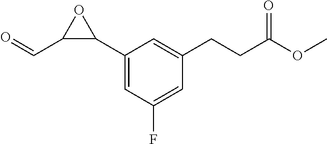 [SMILES: COC(=O)CCc1cc(F)cc(C2OC2C=O)c1] | methyl 3-(3-fluoro-5- (3-formyloxiran-2- yl)phenyl)propanoate | | 253 |
| 26-6 | 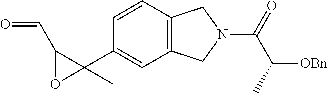 [SMILES: C[C@@H](OCc1ccccc1)C(=O)N1Cc2ccc(C3(C)OC3C=O)cc2C1] | 3-(2-((R)-2- (benzyloxy)propanoyl) isoindolin-5-yl)-3- methyloxirane-2- carbaldehyde | (400 MHz, CDCl₃) δ 9.57 (dd, J = 4.8, 1.4 Hz, 1H), 7.41-7.28 (m, 7H), 7.23-7.19 (m, 1H), 4.96-4.75 (m, 4H), 4.65 (d, J = 11.8 Hz, 1H), 4.49 (d, J = 11.8 Hz, 1H), 4.31 (q, J = 6.6 Hz, 1H), 3.29 (t, J = 5.0 Hz, 1H), 1.84 (d, J = 4.3 Hz, 3H), 1.49 (d, J = 6.7 Hz, 3H). | |

### Intermediate 27

### Methyl (E)-3-(3-(3-acetoxyprop-1-en-1-yl)phenyl)propanoate

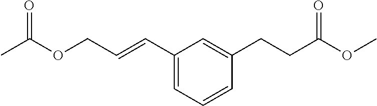 [SMILES: COC(=O)CCc1cccc(/C=C/COC(C)=O)c1]

To a mixture of Pd(OAc)₂ (555 mg, 2.47 mmol) and silver carbonate (4.08 g, 1-4.8 mmnol) in toluene (250 mL) was added methyl 3-(3-bromophenyl)propanoate (6.0 g, 24.7 mmol) followed by allylic acetate (7.41 g, 74.1 mmol) and the resulting mixture was heated at 110° C. for 16 hours. After evaporation, the residue was purified by flash silica gel column chromatography (petroleum ether/ether, v/v, 10/1-4/1) to afford the title compound as colorless oil (2.05 g, 44%). MS m/z: 263 m/z [M+H]⁺.

### Intermediate 28

### 2-Fluoro-5-((6-fluoro-4-methyl-1H-indol-5-yl)oxy)benzohydrazide

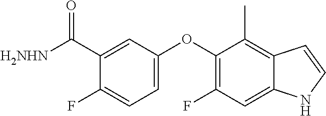

Hydrazine hydrate (1 mL) was added to a 2 mL microwave vial containing a solution of methyl 2-fluoro-5-((6-fluoro-4-methyl-1H-indol-5-yl)oxy)benzoate (Intermediate 6-11, 350 mg, 1.1 mmol) in methanol (1 mL). The reaction mixture was stirred at 130° C. for two hours in a microwave reactor and concentrated. The resulting residue was purified by flash chromatography over silica (0-10% methanol in dichloromethane containing 0.5% concentrated ammonia) to afford the title compound as a white solid (320 mg). MS: 318 m/z [M+H]⁺.

The following intermediates were prepared utilizing similar procedures described for Intermediate 28.

| No. | Structure | Name | MS m/z [M + H]⁺ |
|---|---|---|---|
| 28-1 | 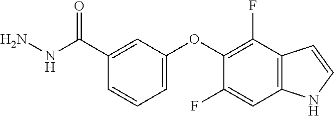 [SMILES: NNC(=O)c1cccc(Oc2c(F)cc3[nH]ccc3c2F)c1] | 3-((4,6-difluoro-1H-indol-5- yl)oxy)benzohydrazide | 304 |
| 28-2 | 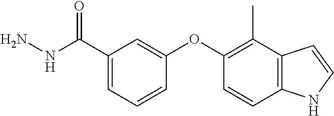 [SMILES: Cc1c(Oc2cccc(C(=O)NN)c2)ccc2[nH]ccc12] | 3-((4-methyl-1H-indol-5- yl)oxy)benzohydrazide | 282 |

### Intermediate 29

### 2-Fluoro-5-(1H-indole-5-carbonyl)benzonitrile

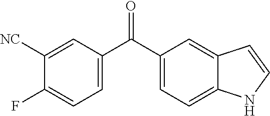 [SMILES: N#Cc1cc(C(=O)c2ccc3[nH]ccc3c2)ccc1F]

To a solution of 2-fluoro-5-(hydroxy(1H-indol-5-yl)methyl)benzonitrile (Intermediate 29B, 0.48, 1.8 mmol) in dichloromethane (15 mL) was added MnO₂ (3.13 g, 36 mmol). The reaction mixture was stirred at room temperature for fix hours, filtered, dried over sodium sulfate and concentrated. The resulting residue was purified by flash chromatography over silica (petroleum ether/ethyl acetate, v/v, 10/1 to 5/1) to give the title compound as a yellow solid (0.36 g, 76%). MS m/z: 265 m/z [M+H]⁺. ¹H NMR (400 MHz, CDCl₃) δ 8.47 (s, 1H), 8.06 (s, 1H), 7.74 (d, J=8.0 Hz, 1H), 7.71 (d, J=8.0 Hz, 1H), 7.52 (s, 1H), 7.50 (s, 1H), 7.35 (d, J=8.0 Hz, 1H), 7.34 (d, J=8.0 Hz, 1H), 6.69 (s, 1H) ppm.

### Intermediate 29A

### 2-Fluoro-5-(hydroxy(1-(triisopropylsilyl)-1H-indol-5-yl)methyl)benzonitrile

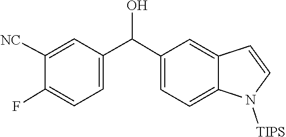

To a solution of 5-bromo-1-(triisopropylsilyl)-1H-indole (2 g, 4.27 mmol) in dry THF (20 mL) at −78° C. was added 2.5M solution of n-butyl lithium in hexanes (2.1 mL) dropwise. The mixture was stirred for 30 minutes at this temperature and a solution of 2-fluoro-5-formylbenzonitrile (1.3 g, 6.41 mmol) in THF (5 mL) was added in one portion. The reaction mixture was stirred at −78° C. for thirty minutes, warmed to room temperature for one hour, quenched with aqueous ammonium chloride (50 mL) and extracted with ethyl acetate (50 mL×3). The combined organic extracts were washed with water and brine, dried over sodium sulfate, filtered and evaporated. The resulting residue was purified by flash chromatography over silica (petroleum ether/ethyl acetate, v/v, 10/1) to afford the title compound as a yellow oil (1.1 g, 46%). MS: 423 m/z [M+H]⁺.

### Intermediate 29B

### 2-Fluoro-5-(hydroxy(1H-indol-5-yl)methyl)benzonitrile

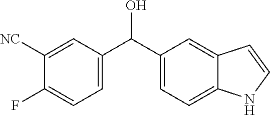 [SMILES: N#Cc1cc(C(O)c2ccc3[nH]ccc3c2)ccc1F]

To a solution of 2-fluoro-5-(hydroxy(1-(triisopropylsilyl)-1H-indol-5-yl)methyl)benzonitrile (1.1 g, 2.6 mmol) in THF (10 mL) was added tetrabutylammonium fluoride (1 M in THF, 3.9 mL). The resulting mixture was stirred at room temperature for two hours, diluted with 15 mL water and then extracted with ethyl acetate (25 mL×2). The combined organic extracts were dried over sodium sulfate, filtered and evaporated. The resulting residue was purified by flash chromatography over silica (petroleum ether/ethyl acetate, v/v, 5/1 to 2/1) to afford the title compound as a yellow oil (0.48 g, 69%). MS: 267 m/z [M+H]⁺. ¹H NMR (400 MHz, CDCl₃) δ 8.28 (br, 1H), 7.71 (d, J=4.8 Hz, 1H), 7.59-7.63 (m, 2H), 7.39 (d, J=8.4 Hz, 1H), 7.08-7.16 (m, 2H), 6.55 (s, 1H), 5.91 (s, 1H), 2.40 (br, 1H) ppm.

### Intermediate 30

### 5-(3-(1H-Pyrazol-3-yl)phenoxy)-4-methyl-1-tosyl-1H-indole

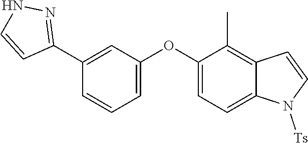 [SMILES: Cc1ccc(S(=O)(=O)n2ccc3c(C)c(Oc4cccc(-c5cc[nH]n5)c4)ccc32)cc1]

A mixture of 5-(3-bromophenoxy)-4-methyl-1-tosyl-1H-indole (Intermediate 7-3, 500 mg, 1.09 mmol), 3-(4,4,5,5-tetramethyl-1,3,2-dioxaborolan-2-yl)-1H-pyrazole (234 mg, 1.21 mmol), Pd(dtbpf)Cl₂ (71 mg, 0.11 mmol) and potassium carbonate (303 mg, 2.19 mmol) in dioxane (8 mL) and water (1 mL) was stirred at 90° C. under nitrogen atmosphere overnight. The reaction mixture was cooled to room temperature, filtered through Celite and washed with ethyl acetate (30 mL×3). The combined organic phase was washed with brine (200 mL), dried over sodium sulfate, filtered and concentrated. The resulting residue was purified by flash chromatography over silica (petroleum ether/ethyl acetate, v/v, 1/1) to afford the title compound as a yellow oil (300 mg, 61%). MS: 444 m/z [M+H]⁺.

The following intermediates were prepared based on the procedures described for Intermediate 30.

| | | | | MS m/z |
|---|---|---|---|---|
| No. | Structure | Name | ¹H NMR | [M + H]⁺ |
| 30-1 | 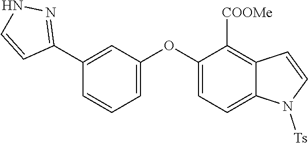 [SMILES: COC(=O)c1c(Oc2cccc(-c3cc[nH]n3)c2)ccc2c1ccn2S(=O)(=O)c1ccc(C)cc1] | methyl 5-(3-(1H- pyrazol-3- yl)phenoxy)-1-tosyl- 1H-indole-4- carboxylate | (400 MHz, CDCl₃) δ 9.06 (s, 1H), 8.11 (d, J = 8.2 Hz, 1H), 7.72 (dd, J = 30.4, 5.4 Hz, 3H), 7.55 (s, 1H), 7.36 (d, J = 19.3 Hz, 2H), 7.25 (d, J = 7.6 Hz, 2H), 7.10-7.02 (m, 2H), 6.88 (s, 1H), 6.67 (s, 1H), 3.81 (s, 3H), 2.36 (s, 3H) ppm. | 488 |
| 30-2 | 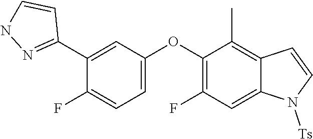 [SMILES: Cc1ccc(S(=O)(=O)n2ccc3c(C)c(Oc4ccc(F)c(-c5cc[nH]n5)c4)c(F)cc32)cc1] | 6-fluoro-5-(4-fluoro-3- (1H-pyrazol-3- yl)phenoxy)-4-methyl- 1-tosyl-1H-indole | (400 MHz, DMSO-d₆). δ 13.03 (s, 1H), 7.98 (d, 2H), 7.90 (d, 1H), 7.78-7.84 (m, 2H), 7.44 (d, 2H), 7.20-7.33 (m, 2H), 6.99 (d, 1H), 6.83-6.85 (m, 1H), 6.62 (d, 1H), 2.36 (s, 3H), 2.31 (s, 3H) ppm. | |

### Intermediate 31

### Methyl 5-(3-(1-(3-iodobenzyl)-1H-pyrazol-3-yl)phenoxy)-1-tosyl-1H-indole-4-carboxylate

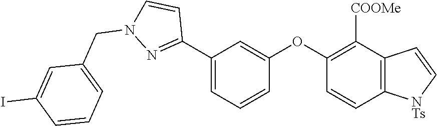 [SMILES: COC(=O)c1c(Oc2cccc(-c3ccn(Cc4cccc(I)c4)n3)c2)ccc2c1ccn2S(=O)(=O)c1ccc(C)cc1]

A mixture of methyl 5-(3-(1H-pyrazol-3-yl)phenoxy)-1-tosyl-1H-indole-4-carboxylate (Intermediate 30-1, 1.5 g, 3.08 mmol), 1-(bromomethyl)-3-iodobenzene (1 g, 3.38 mmol) and potassium carbonate (850.44 mg, 6.15 mmol) in DMF (10 mL) was stirred at room temperature overnight. The solvent was evaporated and the residue was purified by flash chromatography over silica (0-100% ethyl acetate in heptane) to afford the title compound as a white solid (1.6 g, 74%). MS: 704 m/z [M+H]⁺.

### Intermediate 32

### 6-Fluoro-4-methyl-5-(3-(4,4,5,5-tetramethyl-1,3,2-dioxaborolan-2-yl)phenoxy)-1H-indole

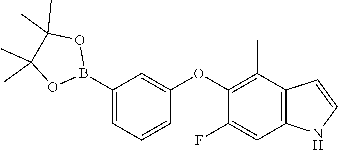 [SMILES: Cc1c(Oc2cccc(B3OC(C)(C)C(C)(C)O3)c2)c(F)cc2[nH]ccc12]

A mixture of 5-(3-bromophenoxy)-6-fluoro-4-methyl-1H-indole (Intermediate 6-12, 2.50 g, 7.84 mmol), 4,4,4′,4′,5,5,5′,5′-octamethyl-2,2′-bi(1,3,2-dioxaborolane) (2.99 g, 11.8 mmol), potassium acetate (1.54 g, 15.7 mmol) and Pd(dppf)Cl₂ (639 mg, 0.78 mmol) in 1,4-dioxane (30 mL) was stirred at 90° C. overnight under nitrogen atmosphere. The reaction mixture was cooled to room temperature, quenched with water (50 mL), filtered through a pad of Celite and extracted with ethyl acetate (50 mL×3). The combined organic extracts were washed with brine, dried over sodium sulfate, filtered and concentrated. The resulting residue was purified by flash chromatography over silica (petroleum ether/ethyl acetate, v/v, 3/1) to afford the title compound as a pale yellow solid (1.78 g, 62%). MS: 368 m/z [M+H]⁺.

### Intermediate 33

### 3-((6-Fluoro-4-(methylthio)-1-tosyl-1H-indol-5-yl)oxy)benzothioamide

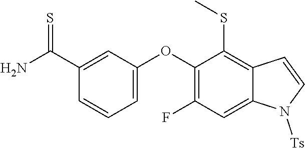 [SMILES: CSc1c(Oc2cccc(C(N)=S)c2)c(F)cc2c1ccn2S(=O)(=O)c1ccc(C)cc1]

To a solution of 3-((6-fluoro-4-(methylthio)-1-tosyl-1H-indol-5-yl)oxy)benzonitrile (Intermediate 23C-1, 1 g, 2.2 mmol) in DMF were added (493 mg), magnesium chloride (418 mg) and water (633 mg, 35 mmol). The reaction mixture was stirred at room temperature for five hours, poured into an ice-water slurry (40 mL) and extracted with ethyl acetate (50 mL×3). The combined organic extracts were washed with brine (50 mL×3), dried over sodium sulfate, filtered and concentrated to afford the title compound as a yellow solid (1.1 g, 71%), which was used for next step without further purification. MS: 487 m/z [M+H]⁺.

The following intermediates were prepared based on the procedures described for Intermediate 33.

| | | | | MS m/z |
|---|---|---|---|---|
| No. | Structure | Name | ¹H NMR | [M + H]⁺ |
| 33-1 | 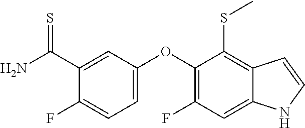 [SMILES: CSc1c(Oc2ccc(F)c(C(N)=S)c2)c(F)cc2[nH]ccc12] | 2-fluoro-5-((6-fluoro-4- (methylthio)-1H-indol-5- yl)oxy)benzothioamide | | 351 |
| 33-2 | 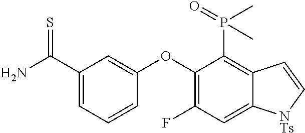 [SMILES: Cc1ccc(S(=O)(=O)n2ccc3c(P(C)(C)=O)c(Oc4cccc(C(N)=S)c4)c(F)cc32)cc1] | 3-((4-(dimethylphosphoryl)- 6-fluoro-1-tosyl-1H-indol-5- yl)oxy)benzothioamide | | 517 |
| 33-3 | 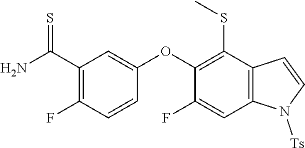 [SMILES: CSc1c(Oc2ccc(F)c(C(N)=S)c2)c(F)cc2c1ccn2S(=O)(=O)c1ccc(C)cc1] | 2-Fluoro-5-((6-fluoro-4- (methylthio)-1-tosyl-1H- indol-5- yl)oxy)benzothioamide | (400 MHz, CD₃OD) δ 7.79- 7.89 (m, 6H), 7.63-7.65 (m, 1H), 7.30-7.32 (m, 2H), 6.89- 7.06 (m, 3H), 2.43 (s, 3H), 2.41 (s, 3H) ppm. | 505 |
| 33-4 | 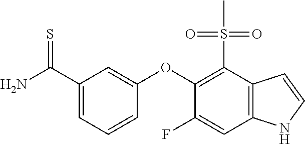 [SMILES: CS(=O)(=O)c1c(Oc2cccc(C(N)=S)c2)c(F)cc2[nH]ccc12] | 3-((6-fluoro-4- (methylsulfonyl)-1H-indol- 5-yl)oxy)benzothioamide | | 365 |

### Intermediate 34

### 6-Fluoro-5-(4-fluoro-3-(1H-pyrazol-3-yl)phenoxy)-4-(methylthio)-1-tosyl-1H-indole

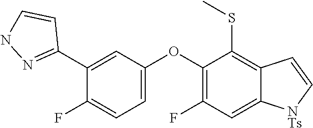

To a solution of 1-(2-fluoro-5-((6-fluoro-4-(methylthio)-1-tosyl-1H-indol-5-yl)oxy)phenyl)ethan-1-one (Intermediate 34A, 140 mg, 0.29 mmol) in toluene (10 mL) was added DMF-DMA (174 mg, 1.45 mmol) and refluxed for two days. The mixture was concentrated to give the crude 3-(dimethylamino)-1-(2-fluoro-5-((6-fluoro-4-(methylthio)-1-tosyl-1H-indol-5-yl)oxy)phenyl)prop-2-en-1-one as an oil (170 mg) which was used in the next step without further purification. MS: 543 m/z [M+H]⁺.

To a solution of the compound above, in ethanol (10 mL) was added hydrazine hydrate (98%, 145 mg, 2.9 mmol), the mixture refluxed for two hours and then concentrated. The residue was purified by flash silica gel column chromatography (0-10% methanol in dichloromethane) to give the title compound as a brown solid (130 mg, 88%). MS: 512 m/z [M+H]⁺.

### Intermediate 34A

### 1-(2-Fluoro-5-((6-fluoro-4-(methylthio)-1-tosyl-1H-indol-5-yl)oxy)phenyl)ethan-1-one

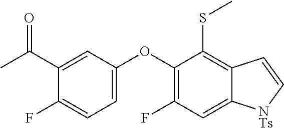 [SMILES: CSc1c(Oc2ccc(F)c(C(C)=O)c2)c(F)cc2c1ccn2S(=O)(=O)c1ccc(C)cc1]

To a solution of 2-fluoro-5-((6-fluoro-4-(methylthio)-1-tosyl-1H-indol-5-yl)oxy)benzonitrile (Intermediate 23C, 940 mg, 2 mmol) in THF (20 mL) was added methylmagnesium bromide (3M in THF, 4 mL, 12 mmol). The reaction mixture was stirred at 50° C. for four hours, quenched with water (30 mL), acidified with 1N hydrochloric acid to pH˜4, stirred for half hour and extracted with ethyl acetate (30 mL×3). The combined organic extract was washed with brine (30 mL×2), dried over sodium sulfate, filtered and concentrated. The resulting residue was purified by silica gel column chromatography (0-50% ethyl acetate in petroleum ether) to give 1-(2-fluoro-5-((6-fluoro-4-(methylthio)-1-tosyl-1H-indol-5-yl)oxy)phenyl)ethan-1-one as an oil (120 mg, 12%). Deprotected 1-(2-fluoro-5-((6-fluoro-4-(methylthio)-1H-indol-5-yl)oxy)phenyl)ethan-1-one (302 mg, 45%) was also obtained and re-protected utilizing TsCl/sodium hydride/DMF conditions to give another 0.32 g of protected product.

1-(2-fluoro-5-((6-fluoro-4-(methylthio)-1-tosyl-1H-indol-5-yl)oxy)phenyl)ethan-1-one: MS: 488 [M+H]⁺.

Deprotected 1-(2-fluoro-5-((6-fluoro-4-(methylthio)-1H-indol-5-yl)oxy)phenyl)ethan-1-one: MS: 334 [M+H]⁺.

### Intermediate 35

### 3-((4-(Dimethylphosphoryl)-6-fluoro-1-tosyl-1H-indol-5-yl)oxy)benzonitrile

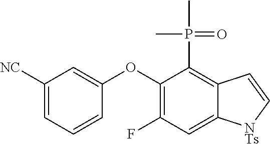 [SMILES: Cc1ccc(S(=O)(=O)n2ccc3c(P(C)(C)=O)c(Oc4cccc(C#N)c4)c(F)cc32)cc1]

To a solution of 3-((4-bromo-6-fluoro-1-tosyl-1H-indol-5-yl)oxy)benzonitrile (Intermediate 7-2, 970 mg, 2 mmol) in DMF (4 mL) were added palladium(II) acetate (45 mg, 0.2 mmol), Xantphos (116 mg, 0.2 mmol) and triethylamine (200 mg, 2.00 mmol) and dimethylphosphine oxide (312 mg, 4 mmol). The reaction mixture was heated at 150° C. for one hour in a microwave reactor. The solvent was evaporated and the resulting residue was purified by flash chromatography over silica (petroleum ether/ethyl acetate, v/v, 16/1 to 4/1) to give the title compound as a light yellow solid (570 mg, 57%). MS: 483 m/z [M+H]⁺.

The following intermediate was prepared based on the procedure described for Intermediate 35.

| No. | Structure | Name | MS m/z. [M + H]⁺ |
|---|---|---|---|
| 35-1 | 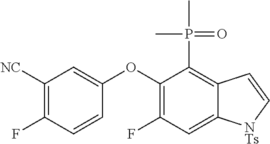 [SMILES: Cc1ccc(S(=O)(=O)n2ccc3c(P(C)(C)=O)c(Oc4ccc(F)c(C#N)c4)c(F)cc32)cc1] | 5-((4-(dimethylphosphoryl)-6-fluoro-1- tosyl-1H-indol-5-yl)oxy)-2- fluorobenzonitrile | 501 |

### Intermediate 36

### 3-((6-Fluoro-4-(methylsulfonyl)-1-tosyl-1H-indol-5-yl)oxy)benzonitrile

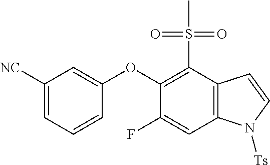 [SMILES: Cc1ccc(S(=O)(=O)n2ccc3c(S(C)(=O)=O)c(Oc4cccc(C#N)c4)c(F)cc32)cc1]

To a solution of 3-((6-fluoro-4-(methylthio)-1-tosyl-1H-indol-5-yl)oxy)benzonitrile (Intermediate 23C-1, 500 mg, 1.1 mmol) in a 1:1 mixture of methanol and THF (50 mL) was added ammonium molybdate tetrahydrate (1 g) in hydrogen peroxide (30% in water, 5 mL). The reaction mixture was stirred at room temperature for three hours, diluted with ethyl acetate (70 mL), washed with water (20 mL×3) and brine (10 mL×2), dried over sodium sulfate and concentrated. The resulting residue was purified by flash chromatography (0-60% ethyl acetate in petroleum ether) to give the title compound as a solid (356 mg, 66%). MS: 485 m/z [M+H]⁺. The following intermediates was prepared based on the procedures described for Intermediate 36.

| No. | Structure | Name | MS m/z [M + H]⁺ |
|---|---|---|---|
| 36-1 | 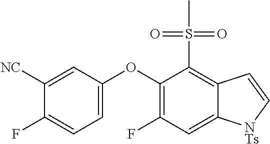 [SMILES: Cc1ccc(S(=O)(=O)n2ccc3c(S(C)(=O)=O)c(Oc4ccc(F)c(C#N)c4)c(F)cc32)cc1] | 2-fluoro-5-((6-fluoro-4-(methylsulfonyl)-1- tosyl-1H-indol-5-yl)oxy)benzonitrile | 503 |

### Intermediate 37

### 5-((4-(Dimethylphosphoryl)-6-fluoro-1H-indol-5-yl)oxy)-2-fluorobenzonitrile

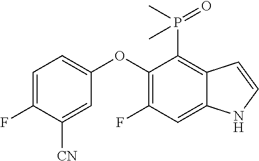 [SMILES: CP(C)(=O)c1c(Oc2ccc(F)c(C#N)c2)c(F)cc2[nH]ccc12]

A mixture of 5-((4-(dimethylphosphoryl)-6-fluoro-1-tosyl-1H-indol-5-yl)oxy)-2-fluorobenzonitrile (Intermediate 35-1, 650 mg, 1.30 mmol) and sodium hydroxide (10 mg, mmol) in methanol (5 mL) was stirred at room temperature for one hour and then concentrated.

The resulting residue was purified by flash chromatography over silica (petroleum ether/ethyl acetate, v/v, 8/1 to 1/1) to give the title compound as a white solid (450 mg, 63%). MS: 347 m/z [M+H]⁺.

The following intermediates were prepared based on the procedures described for Intermediate 36.

| No. | Structure | Name | MS m/z [M + H]⁺ |
|---|---|---|---|
| 37-1 | 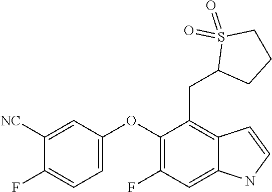 [SMILES: N#Cc1cc(Oc2c(F)cc3[nH]ccc3c2CC2CCCS2(=O)=O)ccc1F] | 5-((4-((1,1-Dioxidotetrahydrothiophen-2- yl)methyl)-6-fluoro-1H-indol-5-yl)oxy)-2- fluorobenzonitrile | 403 |
| 37-2 | 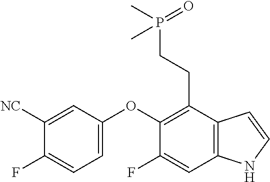 [SMILES: CP(C)(=O)CCc1c(Oc2ccc(F)c(C#N)c2)c(F)cc2[nH]ccc12] | 5-((4-(2-(dimethylphosphoryl)ethyl)-6- fluoro-1H-indol-5-yl)oxy)-2- fluorobenzonitrile | 375 |

### Intermediate 38

### 1-Bromo-4,4-difluoro-3-(3-iodophenyl)-3-methylbutan-2-one

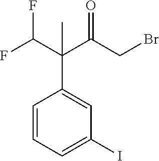 [SMILES: CC(C(=O)CBr)(c1cccc(I)c1)C(F)F]

Exchanging 2-(3-(3-methoxy-3-oxopropyl)phenyl)acetic acid (Intermediate 21B) for 3,3-difluoro-2-(3-iodophenyl)-2-methylpropanoic acid (Intermediate 38D, 900 mg, 2.33 mmol), the reaction procedure described for Intermediate 21 was used to prepare the title compound as an oil (370 mg, 34%). ¹H NMR (400 MHz, DMSO-d₆) δ 7.78-7.80 (d, 1H), 7.68 (s, 1H), 7.36-7.34 (d, 1H), 7.27-7.23 (m, 1H), 6.81-6.54 (t, 1H), 4.59-4.55 (d, 1H), 4.30-4.26 (d, 1H), 1.70 (s, 3H) ppm.

### Intermediate 38A

### Methyl 3-hydroxy-2-(3-iodophenyl)-2-methylpropanoate

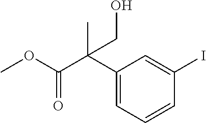

To a solution of 3-hydroxy-2-(3-iodophenyl)-2-methylpropanoic acid (7.44 g, 24.3 mmol) in methanol (150 mL) was added thionyl chloride (15 mL). The reaction mixture was stirred at room temperature, diluted with water, and extracted with ethyl acetate. The combined organic extracts were washed with brine, dried over sodium sulfate, filtered and concentrated. The resulting residue was purified by flash chromatography over silica (0-35% ethyl acetate in petroleum ether) to give the title compound as a solid (6.4 g, 82%). MS: 343 m/z [M+Na]⁺.

### Intermediate 38B

### Methyl 2-(3-iodophenyl)-2-methyl-3-oxopropanoate

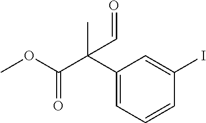

To a solution of methyl 3-hydroxy-2-(3-iodophenyl)-2-methylpropanoate (Intermediate 38A, 6.4 g, 20 mmol) in dichloromethane (100 mL) was added Dess-Martin periodinane (10.2 g, 24 mmol) portion wise at 0° C. The reaction mixture was stirred at room temperature for one hour, diluted with dichloromethane (100 mL), washed with aqueous sodium sulfite and saturated sodium bicarbonate, dried over sodium sulfate, filtered and concentrated. The resulting residue was purified by flash chromatography over silica (0-30% ethyl acetate in petroleum ether) to give the title compound as a solid (4.5 g, 71%). ¹H NMR (400 MHz, DMSO-d₆) δ 9.821 (br s, 1H), 7.76-7.60 (m, 2H), 7.32-7.21 (m, 2H), 3.74 (s, 3H), 1.62 (s, 3H) ppm.

### Intermediate 38C

### Methyl 3,3-difluoro-2-(3-iodophenyl)-2-methylpropanoate

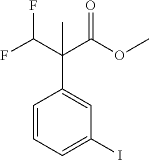 [SMILES: COC(=O)C(C)(c1cccc(I)c1)C(F)F]

To a mixture of methyl 2-(3-iodophenyl)-2-methyl-3-oxopropanoate (Intermediate 38B, 4.5 g, 14.1 mmol) in dry dichloromethane (50 mL) was added DAST (9.34 mL, 70.7 mmol) at 0° C. The reaction mixture was stirred at room temperature overnight, basified to pH˜8 with aqueous sodium bicarbonate and extracted with dichloromethane (50 mL×2). The combined organic extracts were washed with brine, dried over sodium sulfate, filtered and evaporated. The resulting residue was purified by flash silica gel column chromatography (20% ethyl acetate in petroleum ether) to give the title compound as an oil (2.2 g, 46%). MS: 341 m/z [M+H]⁺. ¹H NMR (400 MHz, DMSO-d₆) δ 7.76-7.74 (d, 1H), 7.70 (s, 1H), 7.42-7.40 (d, 1H), 7.24-7.20 (t, 1H), 6.83-6.55 (t, 1H), 3.70 (s, 3H), 1.62 (s, 3H) ppm.

### Intermediate 38D

### 3,3-Difluoro-2-(3-iodophenyl)-2-methylpropanoic acid

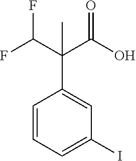

To a solution of methyl 3,3-difluoro-2-(3-iodophenyl)-2-methylpropanoate (Intermediate 38C, 2.3 g, 6.76 mmol) in a 1:1 mixture of THF and water (40 mL) was added lithium hydroxide (1.38 g, 33.8 mmol). The reaction mixture was stirred at room temperature for 1.5 hours, acidified with 1N hydrochloric acid to pH˜4, diluted with ethyl acetate, washed with brine, dried over sodium sulfate, filtered and concentrated. The residue was purified by flash silica gel column chromatography (0-30% ethyl acetate in petroleum ether) to give the title compound as a solid (1.8 g, 82%). MS: 327 m/z [M+H]⁺. ¹H NMR (400 MHz, DMSO-d₆) δ 13.56 (br s, 1H), 7.75-7.73 (m, 2H), 7.46-7.44 (m, 1H), 7.24-7.20 (t, 1H), 6.78-6.51 (t, 1H), 1.56 (s, 3H) ppm.

### Intermediate 39

### Ethyl 3-(3-(4-(2-bromoacetyl)-1-(2,2,2-trifluoroethyl)piperidin-4-yl)phenyl)propanoate

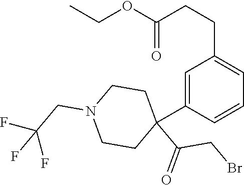 [SMILES: CCOC(=O)CCc1cccc(C2(C(=O)CBr)CCN(CC(F)(F)F)CC2)c1]

Exchanging 2-(3-(3-methoxy-3-oxopropyl)phenyl)acetic acid (intermediate 21B) for 4-(3-(3-ethoxy-3-oxopropyl)phenyl)-1-(2,2,2-trifluoroethyl)piperidine-4-carboxylic acid (900 mg, 2.33 mmol), the reaction procedure described for Intermediate 21 was used to prepare the title compound (370 mg, 34%) as an oil. MS: 464, 466 m/z [M+H]⁺.

### Intermediate 39A

### 4-(3-Bromophenyl)-1-(2,2,2-trifluoroethyl)piperidine-4-carbonitrile

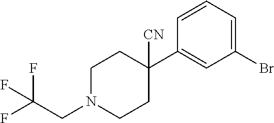 [SMILES: N#CC1(c2cccc(Br)c2)CCN(CC(F)(F)F)CC1]

To a solution of 2,2,2-trifluoroethyl trifluoromethanesulfonate (4.2 g, 18 mmol) in acetone (40 mL) were added potassium carbonate (4.6 g, 33 mmol) and 4-(3-bromophenyl)piperidine-4-carbonitrile hydrochloride (5.1 g, 16.6 mmol). The reaction mixture was refluxed for five hours and concentrated to remove acetone. The residue was dissolved in 50 mL of ethyl acetate, washed with water and brine, dried over sodium sulfate, filtered and concentrated. The resulting crude product was purified by flash silica gel column chromatography (0-10% ethyl acetate in petroleum ether) to give the title compound as colorless oil (4.9 g, 76%). MS: 347, 349 m/z [M+H]⁺.

### Intermediate 39B

### 4-(3-Bromophenyl)-1-(2,2,2-trifluoroethyl)piperidine-4-carboxylic acid

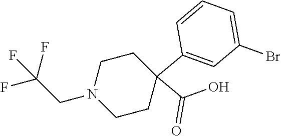 [SMILES: O=C(O)C1(c2cccc(Br)c2)CCN(CC(F)(F)F)CC1]

To a solution of 4-(3-bromophenyl)-1-(2,2,2-trifluoroethyl)piperidine-4-carbonitrile (Intermediate 39A, 4.9 g, 14 mmol) in ethanol (50 mL) and water (10 mL) was added potassium hydroxide (15.86 g, 283 mmol). The reaction mixture was refluxed overnight, quenched with ice, acidified to pH˜7, and extracted with ethyl acetate (100 mL×2). The combined organic extracts were washed with water, brine, dried over sodium sulfate, filtered and concentrated. The resulting residue was purified by flash silica gel column chromatography (0-10% ethyl acetate in petroleum ether) to give the title compound as a white solid (4.9 g, 94%). MS: 366, 368 m/z [M+H]⁺.

### Intermediate 39C

### (E)-4-(3-(3-Ethoxy-3-oxoprop-1-en-1-yl)phenyl)-1-(2,2,2-trifluoroethyl)piperidine-4-carboxylic acid

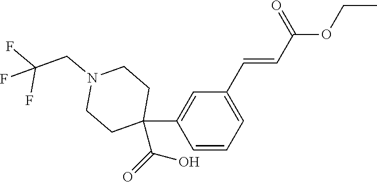 [SMILES: CCOC(=O)/C=C/c1cccc(C2(C(=O)O)CCN(CC(F)(F)F)CC2)c1]

To a stirred solution of 4-(3-bromophenyl)-1-(2,2,2-trifluoroethyl)piperidine-4-carboxylic acid (Intermediate 39B, 4.9 g, 0.013 mol), ethyl acrylate (2.68 g, 0.027 mol) and triethylamine (9.3 mL, 0.065 mmol) in DMF (50 mL) was added tri(o-tolyl)phosphine (0.8 g, 2.7 mmol) and palladium(II) acetate (300 mg, 1.3 mmol). The reaction mixture was heated at 110° C. overnight, cooled to room temperature, diluted ethyl acetate (200 mL), washed with water, brine, dried over sodium sulfate and concentrated. The resulting residue was purified by flash silica gel column chromatography (0-100% methanol in dichloromethane) to give the title compound as a white solid (4.6 g, 92%). MS: 386 m/z [M+H]⁺.

### Intermediate 39D

### 4-(3-(3-Ethoxy-3-oxopropyl)phenyl)-1-(2,2,2-trifluoroethyl)piperidine-4-carboxylic acid

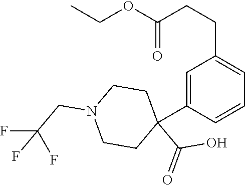 [SMILES: CCOC(=O)CCc1cccc(C2(C(=O)O)CCN(CC(F)(F)F)CC2)c1]

To a solution of (E)-4-(3-(3-ethoxy-3-oxoprop-1-en-1-yl)phenyl)-1-(2,2,2-trifluoroethyl)piperidine-4-carboxylic acid (Intermediate 39C, 1 g, 2.6 mmol) in methanol (10 mL) was added Pd/C (500 mg, 10% on carbon). The reaction mixture was stirred at 30° C. under hydrogen overnight, filtered through a pad of celite, washed the filter cake with methanol. The filtrate was concentrated to give the title compound as white solid (900 mg, 89%), which was used without further purification. MS: 388 [M+H]⁺.

### Intermediate 40

### Ethyl 3-(3-(4-(2-bromoacetyl)-1,1-dioxidotetrahydro-2H-thiopyran-4-yl)phenyl)propanoate

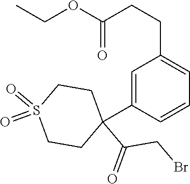 [SMILES: CCOC(=O)CCc1cccc(C2(C(=O)CBr)CCS(=O)(=O)CC2)c1]

Exchanging 2-(3-(3-methoxy-3-oxopropyl)phenyl)acetic acid (intermediate 21B) for 4-(3-(3-ethoxy-3-oxopropyl)phenyl)tetrahydro-2H-thiopyran-4-carboxylic acid 1,1-dioxide (Intermediate 40C, 300 mg, 0.85 mmol), the reaction procedure described for Intermediate 21 was used to prepare the title compound (292 mg, 80%) as a pale yellow oil. MS: 431, 433 m/z [M+H]⁺.

### Intermediate 40A

### 4-(3-Bromophenyl)tetrahydro-2H-thiopyran-4-carboxylic acid

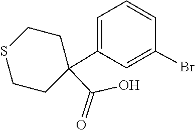 [SMILES: O=C(O)C1(c2cccc(Br)c2)CCSCC1]

Exchanging 1-chloro-2-(chloromethoxy)ethane for bis(2-chloroethyl)sulfane, the reaction procedures described for Intermediate 21-1A and 21-1B were followed to prepare the title compound as an oil (370 mg, 34%). MS: 301, 303 m/z [M+H]⁺.

### Intermediate 40B

### 4-(3-Bromophenyl)tetrahydro-2H-thiopyran-4-carboxylic acid 1,1-dioxide

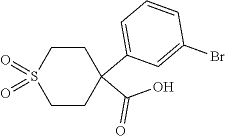 [SMILES: O=C(O)C1(c2cccc(Br)c2)CCS(=O)(=O)CC1]

To a solution of 4-(3-bromophenyl)tetrahydro-2H-thiopyran-4-carboxylic acid (900 mg, 3.0 mmol) in a 1:1 mixture of methanol and THF (90 mL) was added a mixture of ammonium molybdate tetrahydrate (3.6 g) in hydrogen peroxide (30% in water, 9 mL). The reaction mixture was stirred at 25° C. overnight. diluted with water, extracted with ethyl acetate. The combined organic extracts were washed with aqueous sodium sulfite and brine, dried over sodium sulfate, filtered and concentrated. The resulting residue was purified by flash silica gel column chromatography (0-60% ethyl acetate in petroleum ether) to give the title compound as pale white solid (788 mg, 80%). MS: 333, 335 m/z [M+H]⁺.

### Intermediate 40C

### 4-(3-(3-Ethoxy-3-oxopropyl)phenyl)tetrahydro-2H-thiopyran-4-carboxylic acid 1,1-dioxide

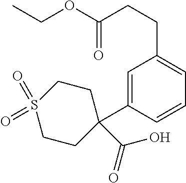 [SMILES: CCOC(=O)CCc1cccc(C2(C(=O)O)CCS(=O)(=O)CC2)c1]

Exchanging 3-(3-bromophenyl)tetrahydrofuran-3-carboxylic acid for 4-(3-Bromophenyl)tetrahydro-2H-thiopyran-4-carboxylic acid 1,1-dioxide, the reaction procedures described for Intermediate 21-1C and 21-1D were followed to prepare the title compound as an oil. MS: 355 m/z [M+H]⁺.

### Intermediate 41

### Ethyl 3-(3-(3-acetyloxiran-2-yl)-2-fluorophenyl)propanoate

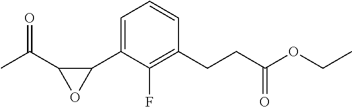 [SMILES: CCOC(=O)CCc1cccc(C2OC2C(C)=O)c1F]

To a solution of ethyl (E)-3-(2-fluoro-3-(3-oxobut-1-en-1-yl)phenyl)propanoate (Intermediate 41B, 1 g, 2.46 mmol) in methanol (10 ml) was added hydrogen peroxide (35% in water, 0.7 ml, 7.38 mmol) at 5° C. over 10 mins, followed by sodium hydroxide (2 M, 1 ml) over 20 mins. The mixture was stirred at room temperature for three hours, quenched with saturated sodium thiosulfate below 40° C., diluted with water (40 ml), extracted with ethyl acetate (40 ml×3), dried over magnesium sulfate, filtered and concentrated. The resulting residue was purified by flash chromatography over silica (20% petroleum ether/ethyl acetate) to afford the title compound (0.5 g, 73%). MS: 281 m/z [M+H]⁺.

### Intermediate 41A

### Ethyl 3-(2-fluoro-3-formylphenyl)propanoate

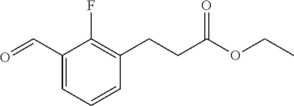 [SMILES: CCOC(=O)CCc1cccc(C=O)c1F]

To a solution of 3-bromo-2-fluoro-benzaldehyde (3 g, 14.8 mmol), palladium (II) acetate (0.332 g, 1.48 mmol), tributylamine (2.74 g, 14.8 mmol) and tetrabutylammonium chloride (4.11 g, 14.8 mmol) in DMF (30 mL) was added 3,3-diethoxyprop-1-ene (1.92 g, 14.8 mmol). The reaction mixture was stirred at 120° C. overnight, cooled to room temperature, was quenched with water (20 ml) and extracted with ethyl acetate (20 ml×3). The combined organic extracts were washed with water, brine, dried over sodium sulfate, filtered and concentrated. The resulting crude product was purified by preparative TLC (ethyl acetate/petroleum ether, v/v, 1/2) to afford the title compound as a light brown solid (2.5 g, 75%). MS: 225 m/z [M+H]⁺.

### Intermediate 41B

### Ethyl (E)-3-(2-fluoro-3-(3-oxobut-1-en-1-yl)phenyl)propanoate

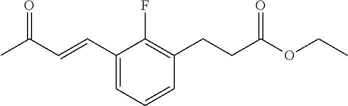 [SMILES: CCOC(=O)CCc1cccc(/C=C/C(C)=O)c1F]

To a solution of ethyl 3-(2-fluoro-3-formyl-phenyl)propanoate (Intermediate 41A, 2 g, 8.92 mmol) in toluene (40 ml) was added 1-(triphenyl-lambda5-phosphanylidene)propan-2-one (2.98 g, 9.37 mmol) and the reaction mixture was stirred at 110° C. over night. Water (20 ml) was added and the mixture was extracted with ethyl acetate (40 ml×3). The combined organic phase was dried with sodium sulfate, filtered and concentrated under reduced pressure. The crude solid was purified by flash chromatography over silica (20% ethyl acetate/petroleum ether) to afford the title compound (3.6 g, 86%). MS: 265 m/z [M+H]⁺.

### Intermediate 42

### 5-((4-(Bromomethyl)-6-fluoro-1-(phenylsulfonyl)-1H-indol-5-yl)oxy)-2-fluorobenzonitrile

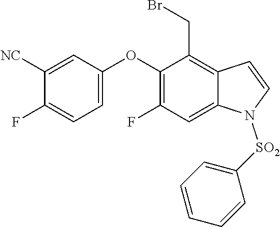 [SMILES: N#Cc1cc(Oc2c(F)cc3c(ccn3S(=O)(=O)c3ccccc3)c2CBr)ccc1F]

To a solution of 2-fluoro-5-((6-fluoro-4-methyl-1-(phenylsulfonyl)-1H-indol-5-yl)oxy)benzonitrile (Intermediate 7-8, 8 g, 18.8 mmol) in dry carbon tetrachloride (650 ml) were added N-bromosuccinimide (3.7 g, 20.7 mmol) and AIBN (0.92 g, 5.6 mmol) at room temperature. The reaction mixture was stirred at 80° C. for five hours, quenched with saturated potassium carbonate solution (200 ml) and extracted with ethyl acetate (100 ml×3). The combined organic extracts were washed with brine, dried over sodium sulfate, filtered and concentrated. The resulting residue was triturated with diethyl ether (50 mL), dried in vacuum to give the title compound as a brown solid (6.4 g, 68%). ¹H NMR (400 MHz, CDCl₃) δ 7.94 (d, J=7.6 Hz, 2H), 7.86 (d, J=10.4 Hz, 1H), 7.72 (d, J=3.6 Hz, 1H), 7.64 (m, 1H), 7.55 (m, 2H), 7.19-7.16 (m, 1H), 7.13 (m, 1H), 7.08-7.04 (m, 1H), 6.82 (d, J=3.6 Hz, 1H), 4.64 (s, 2H) ppm.

### Intermediate 43

### 5-(Benzo[d]thiazol-5-yloxy)-2-fluorobenzimidamide

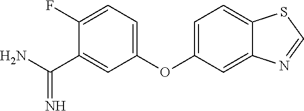 [SMILES: N=C(N)c1cc(Oc2ccc3scnc3c2)ccc1F]

A mixture of 5-(benzo[d]thiazol-5-yloxy)-2-fluorobenzonitrile (Intermediate 43A, 880 mg, 3.25 mmol), diisopropylethylamine (2.51 g, 19.5 mmol) and hydroxylamine (811 mg, 9.77 mmol) in ethanol (5 mL) was heated at 50° C. for four hours and concentrated to give 5-(benzo[d]thiazol-5-yloxy)-2-fluoro-N-hydroxybenzimidamide as a yellow oil (880 mg, 80%). MS: 304 m/z [M+H]⁺.

A mixture of 5-(benzo[d]thiazol-5-yloxy)-2-fluoro-N-hydroxybenzimidamide (800 mg, 2.64 mmol) and acetic anhydride (1.07 g, 10.5 mmol) in acetic acid (50 mL) was stirred for four hours and then concentrated. The resulting residue was dissolved in 10 mL of acetic acid and Pd/C (500 mg) was added. The reaction mixture was stirred under hydrogen atmosphere for 18 hours, filtered through a pad of Celite and rinsed with ethyl acetate. The filtrate was washed with saturated sodium bicarbonate and brine, dried over sodium sulfate, filtered and concentrated to afford the title compound as yellow oil (660 mg, 90%), which was used without further purification. MS: 288 m/z [M+H]⁺.

### Intermediate 43A

### 5-(Benzo[d]thiazol-5-yloxy)-2-fluorobenzonitrile

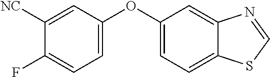 [SMILES: N#Cc1cc(Oc2ccc3scnc3c2)ccc1F]

A mixture of 2-fluoro-5-hydroxybenzonitrile (569 mg, 4.19 mmol), benzo[d]thiazol-5-ylboronic acid (700 mg, 2.79 mmol), Cu(OAc)₂ (100 mg, 0.56 mmol) and triethylamine (563 mg, 5.58 mmol) in dichloromethane (5 mL) was stirred for 18 hours at room temperature. The reaction mixture was diluted with dichloromethane (50 mL), washed with water and brine, dried over magnesium sulfate and evaporated to give the title compound as a white solid (0.88 g, 88%). The product was used in the next step without further purification. MS: 271 m/z [M+H]⁺.

The following intermediates was prepared based on the procedures described for Intermediate 43.

| No. | Structure | Name | MS m/z [M + H]⁺ |
|---|---|---|---|
| 43-1 | 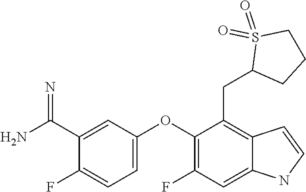 [SMILES: N=C(N)c1cc(Oc2c(F)cc3[nH]ccc3c2CC2CCCS2(=O)=O)ccc1F] | 5-((4-((1,1-Dioxidotetrahydrothiophen- 2-yl)methyl)-6-fluoro-1H-indol-5- yl)oxy)-2-fluorobenzimidamide | 420 |
| 43-2 | 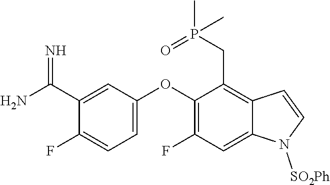 | 5-((4-((dimethylphosphoryl)methyl)-6- fluoro-1-(phenylsulfonyl)-1H-indol-5- yl)oxy)-2-fluorobenzimidamide | 518 |
| 43-3 | 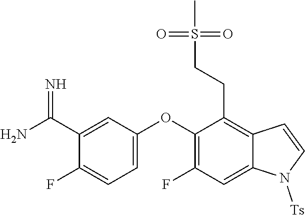 [SMILES: Cc1ccc(S(=O)(=O)n2ccc3c(CCS(C)(=O)=O)c(Oc4ccc(F)c(C(=N)N)c4)c(F)cc32)cc1] | 2-fluoro-5-((6-fluoro-4-(2- (methylsulfonyl)ethyl)-1-tosyl-1H-indol- 5-yl)oxy)benzimidamide | 548 |
| 43-4 | 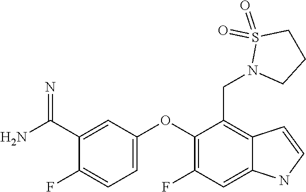 [SMILES: N=C(N)c1cc(Oc2c(F)cc3[nH]ccc3c2CN2CCCS2(=O)=O)ccc1F] | 5-((4-((1,1-dioxidoisothiazolidin-2- yl)methyl)-6-fluoro-1H-indol-5-yl)oxy)- 2-fluorobenzimidamide | 421 |

### Intermediate 44

### Ethyl 2-(3-(4-bromo-3-oxobutan-2-yl)-2-fluorophenyl)acetate

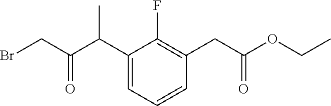 [SMILES: CCOC(=O)Cc1cccc(C(C)C(=O)CBr)c1F]

Exchanging 2-(3-(3-methoxy-3-oxopropyl)phenyl)acetic acid for 2-(3-(2-ethoxy-2-oxoethyl)-2-fluorophenyl)propanoic acid (intermediate 44D), The procedure described for intermediate 21 was used to the title compound as a yellow oil. MS: 331, 333 m/z [M+H]⁺.

### Intermediate 44A

### 2-(Trimethylsilyl)ethyl 2-(3-bromo-2-fluorophenyl)acetate

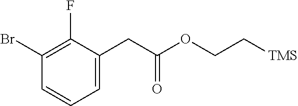 [SMILES: C[Si](C)(C)CCOC(=O)Cc1cccc(Br)c1F]

To a mixture of 2-(3-bromo-2-fluorophenyl)acetic acid (10 g, 42.9 mmol) in dichloromethane (300 mL) were added 2-(trimethylsilyl) ethanol (6.09 g, 51.5 mmol), N, N′-dicyclohexylmethanediimine (10.6 g, 51.5 mmol) and N, N-dimethylpyridin-4-amine (1.05 g, 8.58 mmol). The reaction mixture was stirred at room temperature for 12 hours, diluted with 100 mL of water and extracted with dichloromethane (100 mL×3). The combined organic extracts were washed with brine, dried over sodium sulfate, filtered and concentrated. The resulting residue was purified by flash chromatography over silica (10% ethyl acetate in petroleum ether) to give the title compound as a yellow oil (10 g, 70%). MS: 355, 357 m/z [M+Na]⁺.

### A. 2-(Trimethylsilyl)ethyl 2-(3-bromo-2-fluorophenyl)propanoate

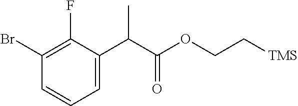 [SMILES: CC(C(=O)OCC[Si](C)(C)C)c1cccc(Br)c1F]

To a solution of 2-(trimethylsilyl)ethyl 2-(3-bromo-2-fluorophenyl)acetate (8 g, 24 mmol) in THF (100 mL) was added LDA (13.2 mL, 2N in THF, 26.4 mmol) dropwise at −78° C. under nitrogen atmosphere. The reaction mixture was stirred at −78° C. for one hour and iodomethane (3.75 g, 26.4 mmol) was added dropwise at −78° C. The mixture was warmed to room temperature, stirred for twelve hours, quenched with ammonium chloride (100 mL) and extracted with ethyl acetate (300 mL×3). The combined organic extracts were washed with saturated sodium chloride (100 mL), dried over sodium sulfate, filtered and concentrated. The resulting residue was purified by flash chromatography over silica (10% ethyl acetate in petroleum ether) to give the title compound as a yellow oil (6 g, 72%). MS: 369, 371 m/z [M+Na]⁺.

### B. 2-(Trimethylsilyl)ethyl 2-(3-(2-ethoxy-2-oxoethyl)-2-fluorophenyl)propanoate

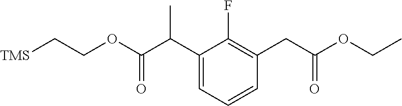 [SMILES: CCOC(=O)Cc1cccc(C(C)C(=O)OCC[Si](C)(C)C)c1F]

To a mixture of 2-(trimethylsilyl)ethyl 2-(3-bromo-2-fluorophenyl)propanoate (6 g, 17.3 mmol) in DMF (30 mL) were added zinc bromide (7.78 g, 34.6 mmol), ethyl 2-tributylstannylacetate (13 g, 34.6 mmol) and palladium (II) tri(o-tolyl)phosphine dichloride (1.36 g, 1.73 mmol). The reaction was stirred at 100° C. for twelve hours under nitrogen, cooled to room temperature and diluted with ethyl acetate. The mixture was washed with saturated sodium chloride (30 mL), dried over sodium sulfate and concentrated. The residue was purified by flash chromatography over silica (10% ethyl acetate in petroleum ether) to give the title compound as a yellow oil (1 g, 16.3%). MS: 377.2 m/z [M+Na]⁺.

### C. 2-(3-(2-Ethoxy-2-oxoethyl)-2-fluorophenyl)propanoic acid

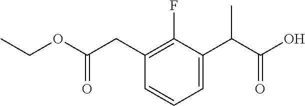 [SMILES: CCOC(=O)Cc1cccc(C(C)C(=O)O)c1F]

To a solution of 2-(trimethylsilyl)ethyl 2-(3-(2-ethoxy-2-oxoethyl)-2-fluorophenyl)propanoate (2 g, 5.64 mmol) in THF (20 mL) was added a 1N solution of tetrabutylammonium fluoride in THF (17 mL, 17 mmol). The reaction mixture was stirred at room temperature for twelve hours under nitrogen, pH was adjusted to ˜3 with 1 N hydrochloric acid solution and extracted with ethyl acetate (100 mL×3). The combined organic extracts were washed with saturated sodium chloride solution (30 mL), dried over sodium sulfate, filtered and concentrated. The residue was purified by flash chromatography over silica (30% ethyl acetate in petroleum ether) to give the title compound as a yellow oil (1 g, 69.7%). MS: 277 m/z [M+Na]⁺.

### Intermediate 45

### 6-Fluoro-5-(3-iodophenoxy)-4-(methylthio)-1H-indole

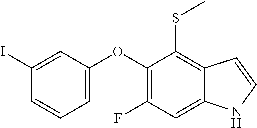 [SMILES: CSc1c(Oc2cccc(I)c2)c(F)cc2[nH]ccc12]

Exchanging 5-(2-bromo-6-fluoro-3-methyl-4-nitrophenoxy)-2-fluorobenzonitrile for (3-fluoro-2-(3-iodophenoxy)-6-methyl-5-nitrophenyl)(methyl)sulfane (2.4 g, 5.73 mmol), the reaction conditions described for intermediate 6 was used to prepare the title compound (1.4 g, 61%) as a reddish solid. ¹H NMR (400 MHz, CDCl₃) δ 8.34 (s, 1H), 7.37-7.33 (m, 1H), 7.29-7.25 (m, 1H), 7.22-7.16 (m, 2H), 7.00-6.96 (m, 1H), 6.88-6.84 (m, 1H), 6.76 (s, 1H), 2.45 (s, 3H) ppm.

### Intermediate 45A

### 3-(2-Bromo-6-fluoro-3-methyl-4-nitrophenoxy)aniline

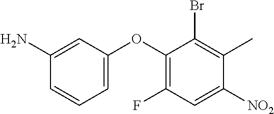 [SMILES: Cc1c([N+](=O)[O-])cc(F)c(Oc2cccc(N)c2)c1Br]

To a solution of 3-bromo-1,2-difluoro-4-methyl-5-nitrobenzene (30.0 g, 0.12 mol) in DMF (300 mL) were added cesium carbonate (77.4 g, 0.24 mmol) and 3-aminophenol (14.3 g, 0.13 mol). The mixture was stirred at room temperature overnight and diluted with water. The precipitate was collected by filtration and dried to give the title compound as a yellow solid (36.0 g, 89%). ¹H NMR (400 MHz, CDCl₃) δ 7.71 (d, J=9.2 Hz, 1H), 7.06 (t, J=7.6 Hz, 1H), 6.42 (d, J=8.8 Hz, 1H), 6.23-6.21 (m, 2H), 3.72 (s, 2H), 2.64 (s, 3H) ppm

### Intermediate 45B

### 3-(6-Fluoro-3-methyl-2-(methylthio)-4-nitrophenoxy)aniline

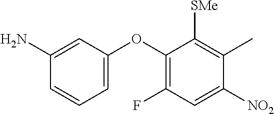 [SMILES: CSc1c(C)c([N+](=O)[O-])cc(F)c1Oc1cccc(N)c1]

Exchanging 5-((4-bromo-6-fluoro-1-tosyl-1H-indol-5-yl)oxy)-2-fluorobenzonitrile for 3-(2-bromo-6-fluoro-3-methyl-4-nitrophenoxy)aniline, the three step reaction sequence described for intermediate 23 (Intermediate 23A to 23C) was used to prepare the title compound as a yellow solid. ¹H NMR (400 MHz, CDCl₃) δ 7.65 (d, J=9.2 Hz, 1H), 7.06 (t, J=7.6 Hz, 1H), 6.40 (d, J=8.8 Hz, 1H), 6.20 (d, J=7.6 Hz, 2H), 3.72 (s, 2H), 2.70 (s, 3H), 2.38 (s, 3H) ppm.

### Intermediate 45C

### (3-Fluoro-2-(3-iodophenoxy)-6-methyl-5-nitrophenyl)(methyl)sulfane

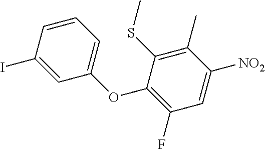 [SMILES: CSc1c(C)c([N+](=O)[O-])cc(F)c1Oc1cccc(I)c1]

To a solution of 3-(6-fluoro-3-methyl-2-(methylthio)-4-nitrophenoxy)aniline (3.0 g, 9.73 mmol, 1.0 eq.) in 3N hydrochloric acid (30 mL) was added sodium nitrite (738 mg, 10.7 mmol, 1.1 eq.) in water (1 mL) at 0° C. The reaction was stirred at 0° C. for 30 min, added potassium iodide (6.46 g, 38.9 mmol, 4.0 eq.) and stirred for another 30 min. The mixture was diluted with 150 mL of ethyl acetate, washed with water, brine, dried over sodium sulfate, filtered and concentrated. The residue was purified by flash chromatography (petroleum ether) to give the title compound as red oil (2.4 g, 59%). ¹H NMR (400 MHz, CDCl₃) δ 7.69-7.66 (m, 1H), 7.45-7.43 (m, 1H), 7.23-7.22 (m, 1H), 7.03 (t, J=8.0 Hz, 1H), 6.86-6.81 (m, 1H), 2.71 (s, 3H), 2.36 (s, 3H) ppm.

### Intermediate 46

### 5-((1-((4-Bromophenyl)sulfonyl)-4-((1,1-dioxidotetrahydrothiophen-2-yl)methyl)-6-fluoro-1H-indol-5-yl)oxy)-2-fluorobenzonitrile

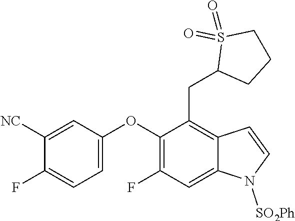

To a solution of thiolane 1,1-dioxide (0.26 g, 2.19 mmol) in THF (10 mL) was added a 2.5 M solution of n-butyl lithium in hexanes (1.2 mL, 3 mmol) under nitrogen at −78° C. The reaction mixture was stirred at −78° C. for one hour. 5-((4-(Bromomethyl)-6-fluoro-1-(phenylsulfonyl)-1H-indol-5-yl)oxy)-2-fluorobenzonitrile (Intermediate 42, 1 g, 1.99 mmol) in THF (7 mL) was added slowly at −78° C. The mixture was stirred at −78° C. for another four hours, quenched with saturated ammonium chloride solution, and extracted with ethyl acetate (30 mL×3). The combined organic extracts were dried and concentrated. The residue was purified by preparative TLC to afford the title compound (0.27 g, 25%). MS: 543 m/z [M+H]⁺.

### Intermediate 47

### 5-((4-((Dimethylphosphoryl)methyl)-6-fluoro-1-(phenylsulfonyl)-1H-indol-5-yl)oxy)-2-fluorobenzonitrile

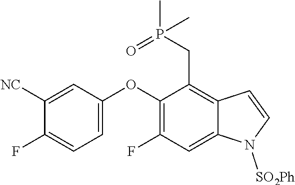

A degassed solution of Pd(OAc)₂ (66.9 mg, 0.298 mmol), Xantphos (345 mg, 0.596 mmol) and diisopropylethylamine (1.53 mL, 8.94 mmol) in THF (30 mL) and water (2 mL) was refluxed for three hours. To this solution was added 5-((4-(bromomethyl)-6-fluoro-1-(phenylsulfonyl)-1H-indol-5-yl)oxy)-2-fluorobenzonitrile (Intermediate 42, 1.5 μg, 2.98 mmol) and dimethylphosphine oxide (302 mg, 3.87 mmol) in THF (10 mL). The mixture was heated under reflux overnight, cooled to room temperature, diluted with 80 mL of water and extracted with ethyl acetate (80 mL×3). The combined organic extracts were washed with brine, dried over sodium sulfate, filtered and concentrated. The crude was purified by flash chromatography over silica (7% methanol in dichloromethane) to give the title compound as a white solid (510 mg, 34%). MS: 501 m/z [M+H]⁺.

### Intermediate 48

### 2-Fluoro-5-((6-fluoro-4-(2-(methylsulfonyl)ethyl)-1-tosyl-1H-indol-5-yl)oxy)benzonitrile

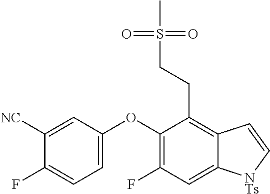 [SMILES: Cc1ccc(S(=O)(=O)n2ccc3c(CCS(C)(=O)=O)c(Oc4ccc(F)c(C#N)c4)c(F)cc32)cc1]

A mixture of 5-((4-(2-bromoethyl)-6-fluoro-1-tosyl-1H-indol-5-yl)oxy)-2-fluorobenzonitrile (0.4 g, 0.75 mmol) and methanesulfinic acid, sodium salt (0.38 g, 3.76 mmol) in DMF (10 mL) was stirred at 90° C. for 12 hours, quenched by water (30 mL) and extracted with ethyl acetate (30 mL×3). The combined organic layers were washed with brine (30 mL), dried over sodium sulfate, filtered and concentrated. The residue was purified by flash chromatography over silica (30% ethyl acetate in petroleum ether) to provide the title compound as a pale solid (0.2 g, 38%). MS: 531 m/z [M+H]⁺.

### Intermediate 48A

### (E)-5-((4-(2-Ethoxyvinyl)-6-fluoro-1H-indol-5-yl)oxy)-2-fluorobenzonitrile

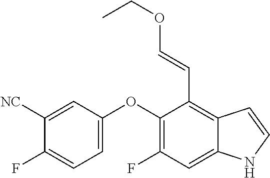 [SMILES: CCO/C=C/c1c(Oc2ccc(F)c(C#N)c2)c(F)cc2[nH]ccc12]

To a solution of 5-((4-bromo-6-fluoro-1H-indol-5-yl)oxy)-2-fluorobenzonitrile (8 g, 22.9 mmol), 2-(2-ethoxyvinyl)-4,4,5,5-tetramethyl-1,3,2-dioxaborolane (4.99 g, 25.2 mmol) and potassium carbonate (4.75 g, 34.4 mmol) in dioxane/water (100 mL/20 mL) was added [1,1′-Bis(diphenylphosphino)ferrocene]dichloropalladium(II) dichloromethane complex (0.94 g, 1.15 mmol). The mixture was stirred at 80° C. for four hours and concentrated. The residue was purified by flash chromatography over silica (20% ethyl acetate in petroleum ether) to provide the title compound as a white solid (7.2 g, 92.3%). MS: 341 m/z [M+H]⁺.

### Intermediate 48B

### (E)-5-((4-(2-Ethoxyvinyl)-6-fluoro-1-tosyl-1H-indol-5-yl)oxy)-2-fluorobenzonitrile

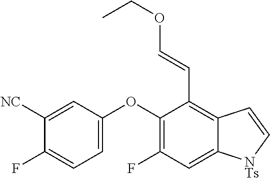 [SMILES: CCO/C=C/c1c(Oc2ccc(F)c(C#N)c2)c(F)cc2c1ccn2S(=O)(=O)c1ccc(C)cc1]

To a mixture of (E)-5-((4-(2-ethoxyvinyl)-6-fluoro-1H-indol-5-yl)oxy)-2-fluorobenzonitrile (5 g, 14.7 mmol) in DMF (60 mL) was added sodium hydride (0.68 g, 17.6 mmol, 60% in oil) portion wise at 0° C. The mixture was stirred for one hour. Then 4-methylbenzenesulfonyl chloride (3.36 g, 17.6 mmol) was added and stirred overnight at room temperature. The mixture was poured into water (100 mL) and extracted with ethyl acetate (3×300 mL). The combined organic extracts were washed with brine (30 mL), dried over sodium sulfate and concentrated. The residue was purified by flash chromatography over silica (20% ethyl acetate in petroleum ether) to provide the title compound as yellow oil (3 g, 41%). MS: 517 m/z [M+Na]⁺.

### Intermediate 48C

### 2-Fluoro-5-((6-fluoro-4-(2-hydroxyethyl)-1-tosyl-1H-indol-5-yl)oxy)benzonitrile

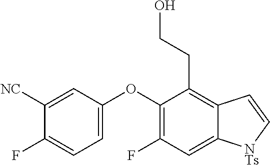 [SMILES: Cc1ccc(S(=O)(=O)n2ccc3c(CCO)c(Oc4ccc(F)c(C#N)c4)c(F)cc32)cc1]

To a mixture of (E)-5-((4-(2-ethoxyvinyl)-6-fluoro-1-tosyl-1H-indol-5-yl)oxy)-2-fluorobenzonitrile (1 g, 2.02 mmol) in THF (10 mL) was added hydrochloric acid in dioxane (10 mL, 4 N). The mixture was stirred at room temperature for four hours, quenched with water (30 mL) and extracted with ethyl acetate (3×30 mL). The combined organic layers were washed with brine (30 mL), dried over sodium sulfate, filtered and concentrated to afford the crude 2-fluoro-5-((6-fluoro-4-(2-oxoethyl)-1-tosyl-1H-indol-5-yl)oxy)benzonitrile as yellow oil (0.8 g). MS: 467 m/z [M+H]⁺.

To a mixture of the crude product obtained above (0.8 g, 1.72 mmol) in methanol (20 mL) was added sodium borohydride (0.13 g, 3.43 mmol). The mixture was stirred at room temperature for four hours. The mixture was quenched with saturated ammonium chloride and extracted with ethyl acetate (100 mL×3). The combined organic layers were washed with brine (30 mL), dried over sodium sulfate and concentrated. The residue was purified by flash chromatography over silica (20% ethyl acetate in petroleum ether) to provide the title compound as yellow oil (0.5 g, 62.2%). MS: 469 m/z [M+H]⁺.

### Intermediate 48D

### 5-((4-(2-Bromoethyl)-6-fluoro-1-tosyl-1H-indol-5-yl)oxy)-2-fluorobenzonitrile

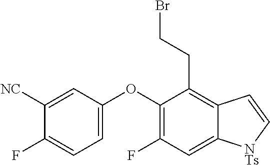 [SMILES: Cc1ccc(S(=O)(=O)n2ccc3c(CCBr)c(Oc4ccc(F)c(C#N)c4)c(F)cc32)cc1]

To a mixture of 2-fluoro-5-((6-fluoro-4-(2-hydroxyethyl)-1-tosyl-1H-indol-5-yl)oxy)benzonitrile (0.5 g, 1.07 mmol) and N-bromosuccinimide (0.285 g, 1.6 mmol) in dichloromethane (20 mL) was added PPh₃ (0.42 g, 1.6 mmol). The reaction was stirred at room temperature for four hours, diluted with water and extracted with ethyl acetate (3×30 mL). The combined organic layers were washed with brine (30 mL), dried over sodium sulfate and concentrated. The residue was purified by flash chromatography over silica (10% ethyl acetate in petroleum ether) to provide the title compound as a white solid (0.4 g, 71%). MS: 531, 533 m/z [M+H]⁺.

### Intermediate 49

### 5-((4-((1,1-Dioxidoisothiazolidin-2-yl)methyl)-6-fluoro-1H-indol-5-yl)oxy)-2-fluorobenzonitrile

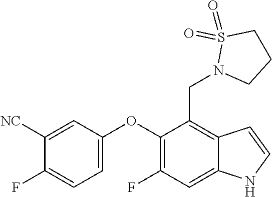 [SMILES: N#Cc1cc(Oc2c(F)cc3[nH]ccc3c2CN2CCCS2(=O)=O)ccc1F]

To a solution of 1,2-thiazolidine 1,1-dioxide (0.27 g, 2.19 mmol) in DMF (8 mL) was add sodium hydride (87 mg, 2.19 mmol, 60% in oil) under nitrogen atmosphere at 0° C. The reaction was stirred at room temperature for one hour. 5-((4-(Bromomethyl)-6-fluoro-1-(phenylsulfonyl)-1H-indol-5-yl)oxy)-2-fluorobenzonitrile (Intermediate 42, 1 g, 1.99 mmol) under nitrogen atmosphere was added to the solution at 0° C. The mixture was stirred at room temperature for three hours. The reaction mixture was quenched with saturated ammonium chloride (15 mL) and extracted with ethyl acetate (30 mL×3). The combined organic layers were dried over sodium sulfate, filtered and concentrated. The residue was purified by flash chromatography over silica to afford the title compound (0.92 g, 85%). MS: 404 m/z [M+H]⁺.

### Intermediate 50

### 5-((4-(2-(Dimethylphosphoryl)ethyl)-6-fluoro-1-tosyl-1H-indol-5-yl)oxy)-2-fluorobenzonitrile

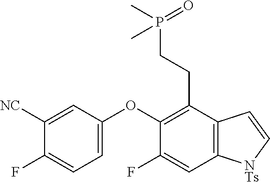 [SMILES: Cc1ccc(S(=O)(=O)n2ccc3c(CCP(C)(C)=O)c(Oc4ccc(F)c(C#N)c4)c(F)cc32)cc1]

To a mixture of 2-fluoro-5-((6-fluoro-1-tosyl-4-vinyl-1H-indol-5-yl)oxy)benzonitrile (Intermediate 8, 3.9 g, 8.66 mmol) in DMSO (40 mL) and water (4 mL) was added potassium hydroxide (0.73 g, 13 mmol) and methylphosphonoylmethane (1.35 g, 17.3 mmol). The mixture was stirred at 50° C. for five hours. Water (200 mL) was added to the solution and then extracted with ethyl acetate (200 mL×2). The organic phase was washed with brine, dried over sodium sulfate, filtered and concentrated to give the crude title compound as a solid (4 g). The sample was used without purification. MS: 529 m/z [M+H]⁺.

### Intermediate 51

### Ethyl 3-(5-(chloromethyl)thiazol-2-yl)propanoate

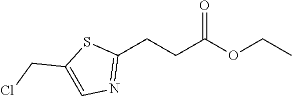

### A. Ethyl (E)-3-(thiazol-2-yl)acrylate

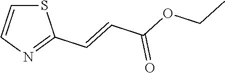

A solution of triethyl phosphonoacetate (26 g, 116 mmol) in THF (50 mL) was added dropwise to a suspension of sodium hydride (4.3 g, 106 mmol) in THF (80 mL) at 0° C. The reaction mixture was stirred at 0° C. for 30 minutes, a solution of 2-formylthiazole (10 g, 88.4 mmol) in THF (80 mL) was added and stirred at room temperature for 16 hours. The reaction mixture was diluted with ethyl acetate (1 L), washed with water and brine, dried over sodium sulfate, filtered and concentrated. The resulting residue was purified by flash chromatography over silica (0-30% ethyl acetate in petroleum ether) to give the title compound as an oil (14.5 g, 90%). MS m/z: 184[M+H]⁺.

### B. Ethyl 3-(thiazol-2-yl)propanoate

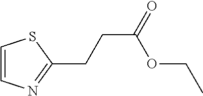

A solution of ethyl (E)-3-(thiazol-2-yl)acrylate (14.5 g, 79.1 mmol) in ethanol (250 mL) was degassed with N₂, Pd/C (1.45 g) was added and stirred at 38° C. under hydrogen atmospheres overnight. The reaction mixture was filtered through a Celite pad and rinsed with ethanol. The filtrate was concentrated to give the title compound as an oil (14 g, 96%). MS m/z: 186 m/z [M+H]⁺.

### C. Ethyl 3-(5-bromothiazol-2-yl)propanoate

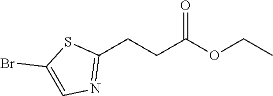

To a solution of ethyl 3-(thiazol-2-yl)propanoate (14 g, 75.6 mmol) in DMF (200 mL) was added N-bromosuccinimide (16.1 g, 90.7 mmol). The mixture was heated at 60° C. overnight, cooled to room temperature, diluted with water (600 mL) and extracted with ethyl acetate (250 mL×3). The combined organic extracts were washed with water (100 mL) and brine (150 mL), dried over sodium sulfate, filtered and concentrated. The resulting residue was purified by purified by flash chromatography over silica (0-30% ethyl acetate in petroleum ether) to give the title compound as an oil (14.9 g, 75%). MS: 264, 266 m/z [M+H]⁺.

### D. Ethyl 3-(5-vinylthiazol-2-yl)propanoate

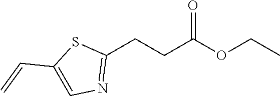

To a degassed solution of ethyl 3-(5-bromothiazol-2-yl)propanoate (2.77 g, 10.5 mmol), 4,4,5,5-tetramethyl-2-vinyl-1,3,2-dioxaborolane (2.9 g, 18.9 mmol) and potassium carbonate (2.9 g, 21 mmol) in dioxane (60 mL) and water (15 mL) was added Pd(dppf)Cl2 (0.41 g, 0.5 mmol) and the reaction mixture was stirred at 100° C. under nitrogen atmosphere overnight. The reaction mixture was cooled to room temperature, diluted with ethyl acetate (150 mL) and washed with water and brine. The organic phase was dried over sodium sulfate, filtered, concentrated and the resulting residue purified by flash chromatography over silica (0-30% ethyl acetate in petroleum ether) to give the title compound as an oil (1.7 g, 77%). MS: 212 m/z [M+H]⁺.

### E. Ethyl 3-(5-formylthiazol-2-yl)propanoate

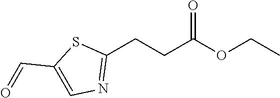

To a solution of ethyl 3-(5-vinylthiazol-2-yl)propanoate (1.7 g, 8 mmol) in THF/water (75 mL) were added sodium periodate (5.15 g, 24 mmol) and catalytic osmium tetroxide, and stirred at room temperature overnight under a nitrogen atmosphere. The reaction mixture was quenched with saturated aqueous sodium thiosulfate (80 mL) and extracted with ethyl acetate (65 mL×3). The combined organic extracts were washed with brine (20 mL), dried over sodium sulfate, filtered and concentrated. The resulting residue was purified by flash chromatography over silica (0-40% ethyl acetate in petroleum ether) to give the title compound as a colorless oil (1.29 g, 76%). MS: 214 m/z [M+H]⁺.

### F. Ethyl 3-(5-(hydroxymethyl)thiazol-2-yl)propanoate

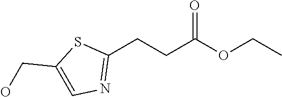

To a solution of ethyl 3-(5-formylthiazol-2-yl)propanoate (1.28 g, 6 mmol) in THF/ethanol (45 mL/15 mL) was added sodium borohydride (0.34 g, 9 mmol) in portions at 0° C. After the addition, the reaction mixture was stirred at 0° C. for two hours, quenched with water and extracted with dichloromethane (50 mL×3). The combined organic extracts were washed with brine, dried over sodium sulfate, filtered and concentrated. The residue was purified by flash chromatography over silica (0-80% ethyl acetate in petroleum ether) to give the title compound as a colorless oil (0.88 g, 67%). MS: 216 m/z [M+H]⁺.

### G. Ethyl 3-(5-(chloromethyl)thiazol-2-yl)propanoate

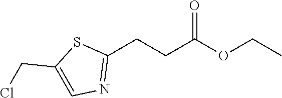

Methanesulfonyl chloride (60 μL, 0.70 mmol) was added to a solution of ethyl 3-(5-(hydroxymethyl)thiazol-2-yl)propanoate (106 mg, 0.5 mmol) and diisopropylethylamine (0.25 mL, 1.5 mmol) in dichloromethane (10 mL). The reaction mixture was stirred at room temperature for two hours, diluted with dichloromethane (80 mL), washed with water (30 mL) and brine, dried over sodium sulfate, filtered and concentrated to give the title compound as colorless oil (0.11 g, 95%). MS: 234 m/z [M+H]⁺.

### Intermediate 52

### (R)-2-(Benzyloxy)-1-(5-bromoisoindolin-2-yl)propan-1-one

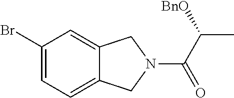 [SMILES: C[C@@H](OCc1ccccc1)C(=O)N1Cc2ccc(Br)cc2C1]

To a stirred solution of 5-bromoisoindoline (900 mg, 3.65 mmol) and (R)-2-(benzyloxy)propanoic acid (658 mg, 3.65 mmol) in DMF (5 mL) were added HATU (2.77 g, 7.30 mmol) and diisopropylethylamine (1.3 mL, 7.30 mmol). The reaction mixture was stirred at room temperature for eighteen hours, quenched with water (30 mL) and extracted with ethyl acetate (50 mL×2). The combined organic extracts were washed with brine (30 mL×3), dried over magnesium sulfate and concentrated. The residue was purified by flash chromatography over silica (0-50% of ethyl acetate in petroleum ether) to give the title compound as yellow oil (530 mg, 36%). MS: 360, 362 m/z [M+H]⁺. ¹H NMR (400 MHz, CDCl3) δ 7.49-6.99 (m, 8H), 4.86-4.08 (m, 7H), 1.49 (d, J=6.7 Hz, 3H).

### Intermediate 53

### Ethyl (S)-2-(3-(4-bromo-3-oxobutan-2-yl-1,1,1-d3)-2-fluorophenyl)acetate

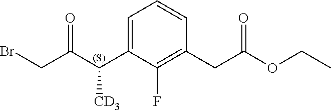

A solution of (S)-2-(3-(2-ethoxy-2-oxoethyl)-2-fluorophenyl)propanoic-3,3,3-d3 acid (Intermediate 53F, 400 mg, 1.24 mmol) in thionyl chloride (2 mL) was stirred at 70° C. for 1 hour and concentrated. To the residue was added toluene and evaporated again to remove trace of thionyl chloride. The residue was dissolved in acetonitrile (6 mL), and treated with trimethylsilyldiazomethane (2 M in hexanes, 3.1 mL, 6.2 mmol) slowly at 0° C. The solution was warmed to room temperature and stirred for 16 hours. To this solution was added hydrobromic acid in water (1.4 mL, 40% w/w) dropwise. The mixture was stirred for 20 minutes until gas bubbling stopped, then quenched with water (5 mL), and extracted with ethyl acetate (3×10 mL). The combined organic layers were washed with brine (2×10 mL), dried over sodium sulfate, filtered, and concentrated. The residue was purified by flash chromatography (40 g silica gel column, eluted with 12% ethyl acetate in petroleum ether) to give the title compound (460 mg, 88%) as a yellow oil. MS (ESI): 334, 336 m/z [M+H]⁺, retention time: 1.79 minutes; purity: 80% (214 nm) (LC-Mass Method: Mobile phase: A: water (0.01% formic acid) B: acetonitrile (0.01% formic acid). Gradient: 5% B for 0.2 minutes, increase to 95% B within 1.3 minutes, 95% B for 1.5 minutes, back to 5% B within 0.01 minutes; Flow Rate: 1.8 mL/minute; Column: Sunfire, 50×4.6 mm, 3.5 μm; Column Temperature: 50° C.).

### Intermediate 53A

### (S)-4-Benzyl-3-(2-(3-bromo-2-fluorophenyl)acetyl)oxazolidin-2-one

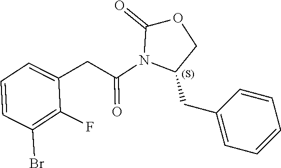

To a stirred and cooled (−78° C.) solution of (S)-4-benzyloxazolidin-2-one (63.88 g, 360 mmol) in tetrahydrofuran (700 mL) was slowly added n-butyllithium (2.5 M in hexanes, 151 mL, 378 mmol). The mixture was stirred at −78° C. for 1 hour to give Mixture A.

To a stirred and cooled (0° C.) solution of 2-(3-bromo-2-fluorophenyl)acetic acid (80.00 g, 343 mmol) in tetrahydrofuran (600 mL) was added N,N-diisopropylethylamine (63 mL, 360 mmol) and pivaloyl chloride (44 mL, 360 mmol). The mixture was warmed to room temperature and stirred for 40 minutes to give Mixture B.

To the stirred and cooled (−78° C.) Mixture A was slowly added Mixture B. The reaction mixture was stirred at −78° C. for 1 hour, then at room temperature for 3 hours. The reaction was quenched with water (800 mL) at 0° C., then concentrated to remove tetrahydrofuran. The aqueous residue was extracted with ethyl acetate (3×500 mL). The combined organic layers were washed with water, brine, dried over sodium sulfate, filtered, and concentrated. The residue was purified by automated flash chromatography (330 g silica gel column, eluted with 0-22% ethyl acetate in petroleum ether) to give the title product (75.00 g, 50%) as a yellow oil. MS (ESI): 392, 394 m/z [M+H]⁺.

### Intermediate 53B

### (S)-4-Benzyl-3-((S)-2-(3-bromo-2-fluorophenyl)propanoyl-3,3,3-d3)oxazolidin-2-one

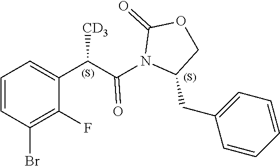

To a stirred and cooled (−78° C.) solution of (S)-4-benzyl-3-(2-(3-bromo-2-fluorophenyl)acetyl)oxazolidin-2-one (Intermediate 53A, 7.00 μg, 14.3 mmol) in tetrahydrofuran (80 mL) was slowly added sodium bis(trimethylsilyl)amide (1 M in tetrahydrofuran, 16 mL, 15.7 mmol). The mixture was stirred at −78° C. for 1 hour, then treated with iodomethane-d₃ (10.35 g, 71.4 mmol), stirred at −78° C. for 1 hour, and at room temperature for 12 hours. The mixture was quenched with water (50 mL) at 0° C. and extracted with ethyl acetate (3×50 mL). The combined organic layers were washed with water, dried over sodium sulfate, filtered, and concentrated. The residue was purified by automated flash chromatography (120 g silica gel column, eluted with 0-22% ethyl acetate in petroleum ether) to give the title compound (5.50 g, 84%) as a colorless oil. MS (ESI): 409, 411 m/z [M+H]⁺.

### Intermediate 53C

### (S)-2-(3-Bromo-2-fluorophenyl)propanoic-3,3,3-d3 acid

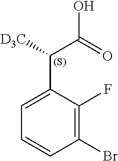

To a stirred and cooled (0° C.) solution of (S)-4-benzyl-3-((S)-2-(3-bromo-2-fluorophenyl)propanoyl-3,3,3-d3)oxazolidin-2-one (Intermediate 53B, 5.50 g, 12.1 mmol) in tetrahydrofuran (40 mL) and water (14 mL) were slowly added a solution of lithium hydroxide monohydrate (761 mg, 18.1 mmol) and hydrogen peroxide (35% in water, 1.1 mL, 12.1 mmol). The mixture was stirred at 0° C. for 2 hours, quenched with saturated sodium sulfite (15 mL), and washed with dichloromethane (2×20 mL). The aqueous layer was acidified with 1 M hydrochloric acid to a pH˜2, and extracted with ethyl acetate (2×40 mL). The combined organic layers were washed with brine, dried over sodium sulfate, filtered, and concentrated to give the title compound (3.10 g, 11.2 mmol, 92%) as a colorless oil. MS (ESI): 250, 252 m/z [M+H]⁺.

### Intermediate 53D

### Benzyl (S)-2-(3-bromo-2-fluorophenyl)propanoate-3,3,3-d3

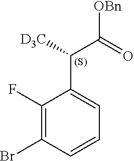

To a stirred solution of (S)-2-(3-bromo-2-fluorophenyl)propanoic-3,3,3-d3 acid (Intermediate 53C, 5.46 g, 21.8 mmol) in acetone (50 mL) were added potassium carbonate (4.53 g, 32.7 mmol) and benzyl bromide (2.9 mL, 24.0 mmol). The mixture was stirred at 40° C. for 3 hours, cooled to room temperature, and quenched with water (100 mL). The solution was extracted with ethyl acetate (3×100 mL). The combined organic layers were washed with water, brine, dried over sodium sulfate, filtered, and concentrated. The residue was purified by automated flash chromatography (120 g silica gel column, eluted with 0-20% ethyl acetate in petroleum ether) to give the title compound (6.40 g, 78%) as a colorless oil. MS (ESI): 340, 342 m/z [M+H]⁺.

### Intermediate 53E

### Benzyl (S)-2-(3-(2-ethoxy-2-oxoethyl)-2-fluorophenyl)propanoate-3,3,3-d3

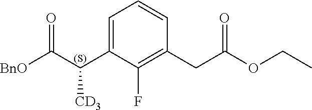

To a stirred solution of potassium 3-ethoxy-3-oxopropanoate (0.75 g, 4.41 mmol) and benzyl (S)-2-(3-bromo-2-fluorophenyl)propanoate-3,3,3-d₃ (1.00 g, 2.94 mmol) in mesitylene (20 mL) were added allylpalladium chloride dimer (65 mg, 0.176 mmol), 2,2′-bis(diphenylphosphino)-1,1′-binaphthyl (329 mg, 0.529 mmol), and 4-dimethylaminopyridine (36 mg, 0.294 mmol) under argon. The mixture was stirred at 160° C. for 2 hours, cooled to room temperature, quenched with water (30 mL) and extracted with ethyl acetate (3×50 mL). The combined organic layers were washed with water, brine, dried over sodium sulfate, filtered, and concentrated. The residue was purified by automated flash chromatography (80 g silica gel column, eluted with 12% ethyl acetate in petroleum ether) to give the title compound (590 mg, 46%) as a colorless oil. MS (ESI): 370 m/z [M+H]⁺.

### Intermediate 53F

### (R) And (S)-2-(3-(2-Ethoxy-2-oxoethyl)-2-fluorophenyl)propanoic-3,3,3-d3 acid

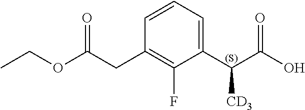

To a stirred solution of benzyl (S)-2-(3-(2-ethoxy-2-oxoethyl)-2-fluorophenyl)propanoate-3,3,3-d₃ (590 mg, 1.53 mmol) in ethanol (6 mL) was added palladium on carbon (wet, 10%, 120 mg). The mixture was stirred at room temperature for 2 hours under hydrogen, filtered through a pad of Celite. The filtrate was concentrated to give the title compound (400 mg, 81%) as an oil. MS (ESI): 280 m/z [M+H]⁺. Chiral purity: 5.2% (RT: 10.79 minutes, R enantiomer); 81.3% (RT: 13.58 minutes, S enantiomer) (Chiral column conditions: AY-H 4.6×250 mm, 5 μM; Hexanes (0.1% diethylamine):ethanol (0.1% diethylamine)=95:5; column temperature: 40° C.; Flow rate: 1.0 mL/minute; Instrument: Shimadzu; Inject Volume: 4 uL; Wavelength: 254 nm).

### Preparation of Compounds

### Example 1

### 3-(3-((3-(3-((4-Methyl-1H-indol-5-yl)oxy)phenyl)-1H-pyrazol-1-yl)methyl)phenyl)propanoic acid

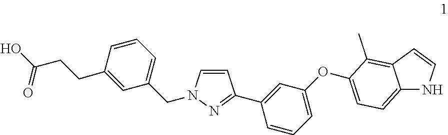 [SMILES: Cc1c(Oc2cccc(-c3ccn(Cc4cccc(CCC(=O)O)c4)n3)c2)ccc2[nH]ccc12]

### A. Ethyl 3-(3-((3-(3-((4-methyl-1-tosyl-1H-indol-5-yl)oxy)phenyl)-1H-pyrazol-1-yl)methyl)phenyl)propanoate

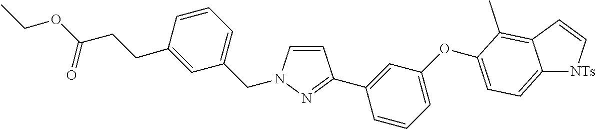 [SMILES: CCOC(=O)CCc1cccc(Cn2ccc(-c3cccc(Oc4ccc5c(ccn5S(=O)(=O)c5ccc(C)cc5)c4C)c3)n2)c1]

A mixture of 5-(3-(1H-pyrazol-3-yl)phenoxy)-4-methyl-1-tosyl-1H-indole (Intermediate 30, 80 mg, 0.18 mmol), ethyl 3-(3-(bromomethyl)phenyl)propanoate (54 mg, 0.19 mmol) and cesium carbonate (118 mg, 0.36 mmol) in acetonitrile (15 mL) was stirred at 80° C. for two hours. The reaction mixture was cooled to room temperature, filtered through Celite and washed with ethyl acetate (50 mL×3). The filtrate was washed with brine (100 mL), dried over sodium sulfate, filtered and concentrated. The resulting residue was purified by flash chromatography over silica (petroleum ether/ethyl acetate, v/v, 1/1) to afford the title compound as a yellow oil (85 mg, 74%). MS: 634 m/z [M+H]⁺.

### B. 3-(3-((3-(3-((4-Methyl-1-tosyl-1H-indol-5-yl)oxy)phenyl)-1H-pyrazol-1-yl)methyl)phenyl)propanoic acid

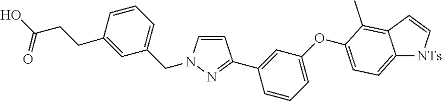 [SMILES: Cc1ccc(S(=O)(=O)n2ccc3c(C)c(Oc4cccc(-c5ccn(Cc6cccc(CCC(=O)O)c6)n5)c4)ccc32)cc1]

To a solution of ethyl 3-(3-((3-(3-((4-methyl-1-tosyl-1H-indol-5-yl)oxy)phenyl)-1H-pyrazol-1-yl)methyl)phenyl)propanoate (86 mg, 0.14 mmol) in THF (10 mL) was added a solution of lithium hydroxide (16 mg, 0.68 mmol) in water (0.4 mL). The reaction mixture was stirred at 65° C. for two hours, cooled to room temperature, diluted with water (10 mL), acidified to pH-2 and extracted with ethyl acetate (30 mL×4). The combined organic extracts were washed with brine, dried over sodium sulfate, filtered and concentrated to afford the title compound as a pink solid (80 mg), which was used directly in the next step. MS: 606 m/z [M+H]⁺.

### C. Example 1, 3-(3-((3-(3-((4-Methyl-1H-indol-5-yl)oxy)phenyl)-1H-pyrazol-1-yl)methyl)phenyl)propanoic acid

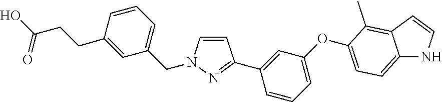 [SMILES: Cc1c(Oc2cccc(-c3ccn(Cc4cccc(CCC(=O)O)c4)n3)c2)ccc2[nH]ccc12]

A mixture of 3-(3-((3-(3-((4-methyl-1-tosyl-1H-indol-5-yl)oxy)phenyl)-1H-pyrazol-1-yl)methyl)phenyl)propanoic acid (80 mg, 0.13 mmol) and potassium carbonate (36 mg, 0.26 mmol) in methanol (5 mL) was irradiated with microwave at 100° C. for 30 minutes. The reaction mixture was cooled to room temperature, quenched with water (30 mL) and extracted with ethyl acetate (50 mL×2). The combined organic extracts were washed with water (30 mL×4) and brine (50 mL×2), dried over sodium sulfate, filtered and concentrated. The resulting residue was purified by preparative HPLC to afford the title compound as a white solid (28 mg, 46%). MS: 452 m/z [M+H]⁺. ¹H NMR (400 MHz, CD₃OD) δ 7.61 (d, J=2.0 Hz, 1H), 7.38 (d, J=7.6 Hz, 1H), 7.29-7.24 (m, 5H), 7.19-7.15 (m, 2H), 7.07 (d, J=7.2 Hz, 1H), 6.83 (d, J=8.8 Hz, 1H), 6.75-6.73 (m, 1H), 6.57 (d, J=2.0 Hz, 1H), 6.52 (d, J=2.8 Hz, 1H), 5.32 (s, 2H), 2.92-2.88 (m, 2H), 2.59-2.56 (m, 2H), 2.36 (s, 3H) ppm.

The following examples were prepared utilizing similar procedures described as in Example 1.

| | | | | LC-MS | |
|---|---|---|---|---|---|
| | | | | Ret time | MS m/z |
| Structure | Ex. No. | Name | ¹H NMR | (min) | [M + H]⁺ |
| 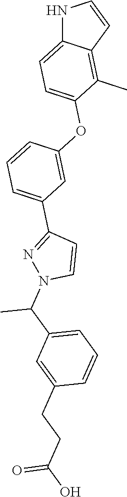 [SMILES: Cc1c(Oc2cccc(-c3ccn(C(C)c4cccc(CCC(=O)O)c4)n3)c2)ccc2[nH]ccc12] | 2 | 3-(3-(1-(3-(3-((4- methyl-1H-indol- 5-yl)oxy)phenyl)- 1H-pyrazol-1- yl)ethyl)phenyl) propanoic acid | (400 MHz, CD₃OD) (400 MHz, CD₃OD) δ 7.64 (d, J = 2.4 Hz, 1H), 7.38 (d, J = 7.6 Hz, 1H), 7.29-7.22 (m, 5H), 7.16-7.15 (m, 2H), 7.06 (d, J = 7.6 Hz, 1H), 6.82 (d, J = 8.8 Hz, 1H), 6.70 (dd, J = 8.0, 1.6 Hz, 1H), 6.54 (d, J = 2.0 Hz, 1H), 6.52 (d, J = 2.8 Hz, 1H), 5.55 (q, J = 6.8 Hz, 1H), 2.89 (t, J = 7.6 Hz, 2H), 2.54 (t, J = 8.0 Hz, 2H), 2.36 (s, 3H), 1.86 (d, J = 7.2 Hz, 3H) ppm | 1.33 | 466 |
| 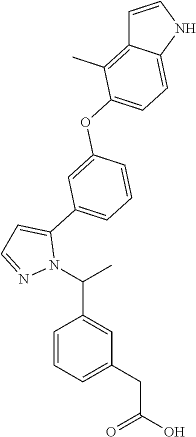 [SMILES: Cc1c(Oc2cccc(-c3ccnn3C(C)c3cccc(CC(=O)O)c3)c2)ccc2[nH]ccc12] | 3 | 2-(3-(1-(5-(3-((4- methyl-1H-indol- 5-yl)oxy)phenyl)- 1H-pyrazol-1- yl)ethyl)phenyl) acetic acid | (400 MHz, CD₃OD) δ 7.55 (s, 1H), 7.36-7.29 (m, 2H), 7.22 (d, J = 8.4 Hz, 1H), 7.12-7.05 (m, 2H), 6.94- 6.92 (m, 3H), 6.77-6.74 (m, 2H), 6.56 (s, 1H), 6.50 (d, J = 2.8 Hz, 1H), 6.31 (s, 1H), 5.52 (q, 1H), 3.45 (s, 2H), 2.28 (s, 3H), 1.76 (d, J = 6.8 Hz, 3H) ppm. | 1.36 | 452 |
| 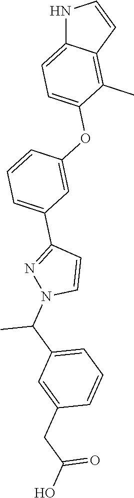 [SMILES: Cc1c(Oc2cccc(-c3ccn(C(C)c4cccc(CC(=O)O)c4)n3)c2)ccc2[nH]ccc12] | 4 | 2-(3-(1-(3-(3-((4- methyl-1H-indol- 5-yl)oxy)phenyl)- 1H-pyrazol-1- yl)ethyl)phenyl) acetic acid | (400 MHz, CD₃OD) δ 7.63 (s, 1H), 7.37 (d, J = 7.6 Hz, 1H), 7.28-7.22 (m, 7H), 7.11 (d, J = 7.2 Hz, 1H), 6.82 (d, J = 8.8 Hz, 1H), 6.69 (d, J = 7.6 Hz, 1H), 6.52 (d, J = 3.6 Hz, 2H), 5.56 (q, J = 6.8 Hz, 1H), 3.57 (s, 2H), 2.35 (s, 3H), 1.86 (d, J = 6.8 Hz, 3H) | 1.38 | 452 |
| 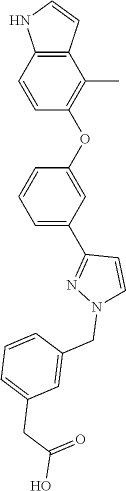 [SMILES: Cc1c(Oc2cccc(-c3ccn(Cc4cccc(CC(=O)O)c4)n3)c2)ccc2[nH]ccc12] | 5 | 2-(3-((3-(3-((4- methyl-1H-indol- 5-yl)oxy)phenyl)- 1H-pyrazol-1- yl)methyl)phenyl) acetic acid | (400 MHz, CD₃OD) δ 7.61 (d, J = 2.4 Hz, 1H), 7.40-7.28 (m, 3H), 7.28-7.17 (m, 5H), 7.12 (d, J = 7.6 Hz, 1H), 6.81 (d, J = 8.6 Hz, 1H), 6.71 (m, 1H), 6.58-6.47 (m, 2H), 5.32 (s, 2H), 3.57 (s, 2H), 2.34 (s, 3H) ppm. | 1.29 | 438 |

### Example 6

### 3-(3-((3-(3-((4-Methyl-1H-indol-5-yl)oxy)phenyl)-1H-pyrazol-1-yl)methyl)phenyl)propan-1-ol

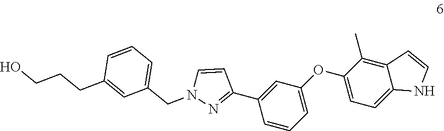 [SMILES: Cc1c(Oc2cccc(-c3ccn(Cc4cccc(CCCO)c4)n3)c2)ccc2[nH]ccc12]

To a stirred solution of 3-(3-((3-(3-((4-methyl-1H-indol-5-yl)oxy)phenyl)-1H-pyrazol-1-yl)methyl)phenyl)propanoic acid (Example 1, 100 mg, 0.23 mmol) in THF (10 mL) was added borane-THF complex (0.8 mL, 0.8 mmol, 1M in THF) at 0° C. The reaction mixture was stirred at room temperature for three hours, quenched with water (20 mL) and extracted with ethyl acetate (20 mL×3). The combined organic extracts were washed with brine (20 mL), dried over sodium sulfate, filtered and concentrated. The resulting residue was purified by preparative HPLC to give the title compound as a white solid (32.2 mg, 33%). ¹H NMR (400 MHz, CD₃OD) δ 7.62 (br s, 1H), 7.38 (d, J=7.6 Hz, 1H), 7.29-7.24 (m, 5H), 7.16 (d, J=7.6 Hz, 1H), 7.11 (s, 1H), 7.06 (d, J=7.6 Hz, 1H), 6.83 (d, J=8.8 Hz, 1H), 6.73 (d, J=7.2 Hz, 1H), 6.58 (br s, 1H), 6.52 (d, J=2.8 Hz, 1H), 5.32 (s, 2H), 3.57-3.54 (m, 2H), 2.68-2.65 (m, 2H), 2.36 (s, 3H), 1.84-1.77 (m, 2H) ppm. LC-MS Retention time: 1.39 minutes. MS: 438 m/z [M+H]⁺.

### Example 7

### Methyl (E)-3-(3-((3-(3-((4-(hydroxymethyl)-1H-indol-5-yl)oxy)phenyl)-1H-pyrazol-1-yl)methyl)phenyl)acrylate

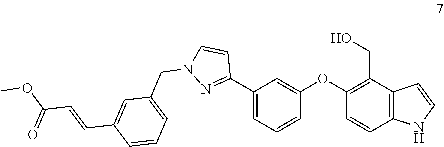 [SMILES: COC(=O)/C=C/c1cccc(Cn2ccc(-c3cccc(Oc4ccc5[nH]ccc5c4CO)c3)n2)c1]

### A. (5-(3-(1-(3-Iodobenzyl)-1H-pyrazol-3-yl)phenoxy)-1-tosyl-1H-indol-4-yl)methanol

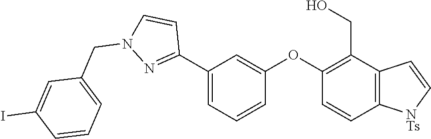 [SMILES: Cc1ccc(S(=O)(=O)n2ccc3c(CO)c(Oc4cccc(-c5ccn(Cc6cccc(I)c6)n5)c4)ccc32)cc1]

To a mixture of methyl 5-(3-(1-(3-iodobenzyl)-1H-pyrazol-3-yl)phenoxy)-1-tosyl-1H-indole-4-carboxylate (Intermediate 31, 0.45 g, 0.64 mmol) in dichloromethane (10 mL) was added DIBAL-H (1.92 mL, 1.9 mmol) at −78° C. The mixture was stirred for one hour and then quenched with saturated solution of Rochelle's salt (10 mL). The mixture was stirred overnight at room temperature and extracted with ethyl acetate. The combined organic extracts were dried over sodium sulfate, filtered and concentrated. The resulting residue was purified by flash chromatography over silica (0-100% ethyl acetate in heptane) to give the title compound as a white solid (0.4 g, 93%). MS: 676 m/z [M+H]⁺.

### B. (5-(3-(1-(3-Iodobenzyl)-1H-pyrazol-3-yl)phenoxy)-1H-indol-4-yl)methanol

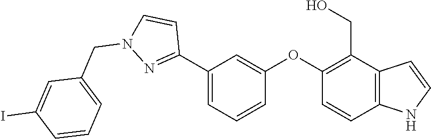 [SMILES: OCc1c(Oc2cccc(-c3ccn(Cc4cccc(I)c4)n3)c2)ccc2[nH]ccc12]

A mixture of (5-(3-(1-(3-iodobenzyl)-1H-pyrazol-3-yl)phenoxy)-1-tosyl-1H-indol-4-yl)methanol (0.4 g, 0.6 mmol) and potassium carbonate (164 mg, 1.18 mmol) in 5 mL of methanol was heated at 130° C. for 15 minutes. The solvent was evaporated and the residue was purified by flash chromatography over silica (0-100% ethyl acetate in heptane) to afford the title compound as a white solid (0.23 g, 75%). MS: 522 m/z [M+H]⁺.

### C. Example 7, Methyl (E)-3-(3-((3-(3-((4-(hydroxymethyl)-1H-indol-5-yl)oxy)phenyl)-1H-pyrazol-1-yl)methyl)phenyl)acrylate

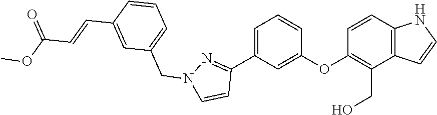 [SMILES: COC(=O)/C=C/c1cccc(Cn2ccc(-c3cccc(Oc4ccc5[nH]ccc5c4CO)c3)n2)c1]

A mixture of (5-(3-(1-(3-iodobenzyl)-1H-pyrazol-3-yl)phenoxy)-1H-indol-4-yl)methanol (0.23 g, 0.44 mmol) and triethylamine (0.62 mL, 4.4 mmol) in DMF (1.5 mL) was degassed for 10 minutes, palladium(II) acetate (5 mg, 22 μmol) and tri-o-tolylphosphine (27 mg, 88 μmol) were added to the microwave vial and sealed. Methyl acrylate (0.052 mL, 0.57 mmol) was added via a syringe and the mixture was heated at 100° C. for 30 minutes. The solvent was removed and the residue was purified by flash chromatography over silica (0-100% ethyl acetate in heptane) to afford the title compound as a white solid (0.18 g, 85%). ¹H NMR (400 MHz, CD₃OD) δ 7.70-7.61 (m, 2H), 7.59-7.45 (m, 2H), 7.43-7.22 (m, 7H), 6.85-6.77 (m, 2H), 6.70 (dd, J=3.2, 0.9 Hz, 1H), 6.60 (d, J=2.4 Hz, 1H), 6.51 (d, J=16.1 Hz, 1H), 5.36 (s, 2H), 4.87 (s, 2H), 3.78 (s, 3H) ppm. LC-MS Retention time: 1.28 min; MS: 480 m/z [M+H]⁺.

### Example 8

### Methyl 3-(3-((3-(3-((4-(hydroxymethyl)-1H-indol-5-yl)oxy)phenyl)-1H-pyrazol-1-yl)methyl)phenyl)propanoate

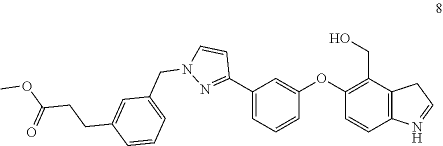 [SMILES: COC(=O)CCc1cccc(Cn2ccc(-c3cccc(Oc4ccc5c(c4CO)CC=N5)c3)n2)c1]

A mixture of methyl 3-(3-((3-(3-((4-(hydroxymethyl)-1H-indol-5-yl)oxy)phenyl)-1H-pyrazol-1-yl)methyl)phenyl)acrylate (Example 7, 0.14 g, 0.29 mmol) and Pd—C (62 mg, 0.058 mmol, 10% on carbon) in 15 mL of ethyl acetate was stirred under hydrogen balloon for five hours. The reaction mixture was then filtered through a pad of Celite and washed with ethyl acetate. The solvent was evaporated and the residue was purified by flash chromatography over silica (0-100% ethyl acetate in heptane) to give the title compound as a white solid (100 mg, 71%). ¹H NMR (400 MHz, CD₃OD) δ 7.61 (d, J=2.4 Hz, 1H), 7.43-7.19 (m, 6H), 7.17-7.02 (m, 3H), 6.85-6.77 (m, 2H), 6.70 (dd, J=3.2, 0.9 Hz, 1H), 6.58 (d, J=2.4 Hz, 1H), 5.30 (s, 2H), 4.88 (s, 2H), 3.57 (s, 3H), 2.88 (t, J=7.6 Hz, 2H), 2.59 (t, J=7.6 Hz, 2H) ppm. LC-MS Retention time: 1.26 minutes. m/z: 482 m/z [M+H]⁺.

### Example 9

### (E)-3-(3-((3-(3-((4-(Hydroxymethyl)-1H-indol-5-yl)oxy)phenyl)-1H-pyrazol-1-yl)methyl)phenyl)acrylic acid

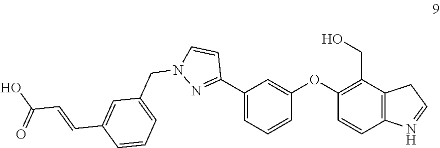 [SMILES: O=C(O)/C=C/c1cccc(Cn2ccc(-c3cccc(Oc4ccc5c(c4CO)CC=N5)c3)n2)c1]

A mixture of methyl (E)-3-(3-((3-(3-((4-(hydroxymethyl)-1H-indol-5-yl)oxy)phenyl)-1H-pyrazol-1-yl)methyl)phenyl)acrylate (25 mg, 52 μmol) and lithium hydroxide (3.8 mg, 0.16 mmol) in THF (2 mL) and water (1 mL) was stirred overnight at room temperature. The solvent was evaporated, the residue was dissolved in 0.5 mL of water, neutralized with 1N HCl. The white precipitate was collected by filtration and dried to afford the title compound (18 mg, 74%). ¹H NMR (400 MHz, CD₃OD) δ 7.72-7.59 (m, 2H), 7.57-7.46 (m, 2H), 7.44-7.20 (m, 8H), 6.86-6.77 (m, 2H), 6.61 (d, J=2.4 Hz, 1H), 6.46 (d, J=16.0 Hz, 1H), 5.37 (s, 2H), 4.87 (s, 2H) ppm. LC-MS Retention time: 1.14 minutes. MS m/z: 466 m/z [M+H]⁺.

### Example 10

### 3-(3-((3-(3-((4-(Hydroxymethyl)-1H-indol-5-yl)oxy)phenyl)-1H-pyrazol-1-yl)methyl)phenyl)propanoic acid

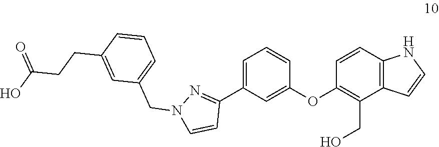 [SMILES: O=C(O)CCc1cccc(Cn2ccc(-c3cccc(Oc4ccc5[nH]ccc5c4CO)c3)n2)c1]

A mixture of methyl 3-(3-((3-(3-((4-(hydroxymethyl)-1H-indol-5-yl)oxy)phenyl)-1H-pyrazol-1-yl)methyl)phenyl)propanoate (Example 8, 100 mg, 207 μmol) and lithium hydroxide (15 mg, 623 μmol) in THF (5 mL) and water (3 mL) was stirred overnight. The solvent was evaporated, the residue was dissolved in 2 mL of water and neutralized with 1N HCl. The white solid was collected by filtration to afford the title compound (90 mg, 93%). ¹H NMR (400 MHz, CD₃OD) δ 7.61 (d, J=2.4 Hz, 1H), 7.43-7.20 (m, 6H), 7.20-7.10 (m, 2H), 7.05 (d, J=7.7 Hz, 1H), 6.85-6.77 (m, 2H), 6.70 (dd, J=3.2, 0.9 Hz, 1H), 6.58 (d, J=2.4 Hz, 1H), 5.30 (s, 2H), 4.88 (s, 2H), 2.88 (t, J=7.7 Hz, 2H), 2.56 (t, J=7.7 Hz, 2H) ppm. LC-MS Retention time: 1.14 minutes. MS: 468 m/z [M+H]⁺.

### Example 11

### 2-(3-((3-(3-((4-(Hydroxymethyl)-1H-indol-5-yl)oxy)phenyl)-1H-pyrazol-1-yl)methyl)phenyl)acetic acid

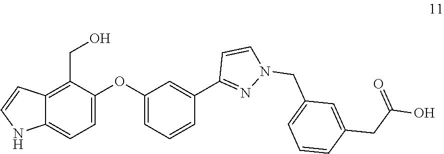 [SMILES: O=C(O)Cc1cccc(Cn2ccc(-c3cccc(Oc4ccc5[nH]ccc5c4CO)c3)n2)c1]

### A. (5-(3-(1H-Pyrazol-3-yl)phenoxy)-1-tosyl-1H-indol-4-yl)methanol

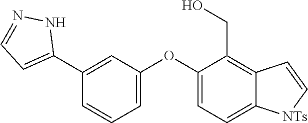 [SMILES: Cc1ccc(S(=O)(=O)n2ccc3c(CO)c(Oc4cccc(-c5ccn[nH]5)c4)ccc32)cc1]

To a solution of methyl 5-(3-(1H-pyrazol-3-yl)phenoxy)-1-tosyl-1H-indole-4-carboxylate (Intermediate 30-1, 1.25 g, 2.56 mmol) in 15 mL of dichloromethane was added DIBAL (12.82 mL, 12.82 mmol, 1 M in hexane) at −78° C. The reaction mixture was allowed to warm to room temperature and stirred for 1.5 hours. The reaction mixture was quenched with saturated Rochelle's salt, stirred overnight until it turned to clear solution and extracted with dichloromethane (50 mL×3). The combined organic extracts were washed with water, brine, dried over sodium sulfate and filtered. After removal of solvent the resulting residue was purified by flash chromatography over silica (0-10% methanol in dichloromethane) to give the title compound as a white solid (843 mg). ¹H NMR (400 MHz, CDCl₃) δ 7.90 (dd, J=9.0, 3.2 Hz, 1H), 7.76 (dq, J=8.4, 2.0 Hz, 2H), 7.67-7.54 (m, 2H), 7.49-7.21 (m, 5H), 7.01-6.86 (m, 3H), 6.58-6.52 (m, 1H), 4.88 (d, J=3.3 Hz, 2H), 2.36 (d, J=3.2 Hz, 3H) ppm. MS: 458 m/z [M−H]⁻.

### B. Methyl 2-(3-((3-(3-((4-(hydroxymethyl)-1H-indol-5-yl)oxy)phenyl)-1H-pyrazol-1-yl)methyl)phenyl)acetate

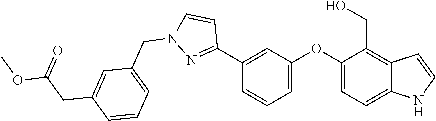 [SMILES: COC(=O)Cc1cccc(Cn2ccc(-c3cccc(Oc4ccc5[nH]ccc5c4CO)c3)n2)c1]

A mixture of (5-(3-(1H-pyrazol-5-yl)phenoxy)-1-tosyl-1H-indol-4-yl)methanol (615 mg, 1.34 mmol), sodium iodide (cat), methyl 2-(3-(bromomethyl)phenyl)acetate (374.16 mg, 1.54 mmol) and potassium carbonate (369.93 mg, 2.68 mmol) in DMF (10 mL) was stirred at room temperature overnight. The solvent was evaporated and the residue was chromatographed (0-100% ethyl acetate in heptane) to give the title compound as a white solid (129 mg, 16%).

### C. Example 11, 2-(3-((3-(3-((4-(Hydroxymethyl)-1H-indol-5-yl)oxy)phenyl)-1H-pyrazol-1-yl)methyl)phenyl)acetic acid

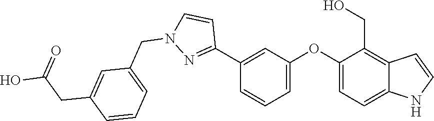 [SMILES: O=C(O)Cc1cccc(Cn2ccc(-c3cccc(Oc4ccc5[nH]ccc5c4CO)c3)n2)c1]

To a 10 mL microwave reaction vial were added methyl 2-(3-((3-(3-((4-(hydroxymethyl)-1-tosyl-1H-indol-5-yl)oxy)phenyl)-1H-pyrazol-1-yl)methyl)phenyl)acetate (25 mg, 0.04 mmol) and lithium hydroxide (25 mg) in methanol (4 mL) and water (2 mL). The reaction mixture was heated in a microwave reactor at 120° C. for 30 minutes and concentrated to remove methanol. The aqueous solution was acidified with 1 N hydrochloric acid to pH˜2, the precipitate collected and dried to afford the title compound as a white solid. LC-MS Retention time: 1.1 minutes, MS m/z: 454 m/z [M+H]⁺.

The following examples were prepared utilizing similar procedures described for Example 11.

| | | | | LC-MS Ret | MS m/z |
|---|---|---|---|---|---|
| Structure | Ex. No | Name | ¹H NMR | time (min) | [M + H]⁺ |
| 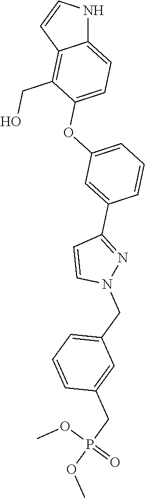 [SMILES: COP(=O)(Cc1cccc(Cn2ccc(-c3cccc(Oc4ccc5[nH]ccc5c4CO)c3)n2)c1)OC] | 12 | dimethyl (3-((3-(3- ((4- (hydroxymethyl)- 1H-indol-5- yl)oxy)phenyl)-1H- pyrazol-1- yl)methyl)benzyl) phosphonate | | 1.06 | 518 |
| 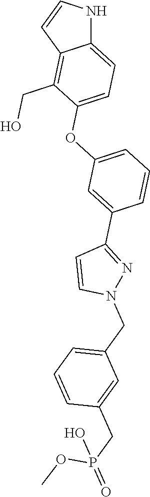 [SMILES: COP(=O)(O)Cc1cccc(Cn2ccc(-c3cccc(Oc4ccc5[nH]ccc5c4CO)c3)n2)c1] | 13 | methyl hydrogen (3- ((3-(3-((4- (hydroxymethyl)- 1H-indol-5- yl)oxy)phenyl)-1H- pyrazol-1- yl)methyl)benzyl) phosphonate | (400 MHz, DMSO-d₆) δ 11.26 (s, 1H), 7.78 (t, J = 2.0 Hz, 1H), 7.37-7.11 (m, 10H), 6.95-6.51 (m, 3H), 5.25 (s, 2H), 4.68 (d, J = 5.1 Hz, 2H), 4.15-4.07 (m, 3H), 3.26 (dd, J = 10.2, 1.6 Hz, 3H) ppm. | 0.94 | 504 |
| 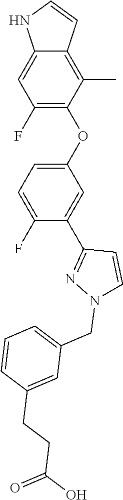 [SMILES: Cc1c(Oc2ccc(F)c(-c3ccn(Cc4cccc(CCC(=O)O)c4)n3)c2)c(F)cc2[nH]ccc12] | 14 | 3-(3-((3-(2-fluoro-5- ((6-fluoro-4-methyl- 1H-indol-5- yl)oxy)phenyl)-1H- pyrazol-1- yl)methyl)phenyl) propanoic acid | ¹H NMR (400 MHz, DMSO-d₆) δ 11.4 (s, 1H), 7.87 (d, J = 2.0 Hz, 1H), 7.39 (s, 1H), 7.28-7.26 (m, 1H), 7.24-7.19 (m, 3H), 7.15-7.13 (m, 2H), 7.01 (d, J = 7.2 Hz, 1H), 6.84-6.80 (m, 1H), 6.63-6.62 (m, 1H), 6.52 (s, 1H), 5.29 (s, 2H), 3.32 (br s, 1H), 2.76 (t, J = 7.6 Hz, 2H), 2.47 (t, J = 7.6 Hz, 2H), 2.32 (s, 3H) ppm. | 1.44 | 488 |

### Example 15

### (E)-3-(3-((3-(3-((4-(Methylcarbamoyl)-1H-indol-5-yl)oxy)phenyl)-1H-pyrazol-1-yl)methyl)phenyl)acrylic acid

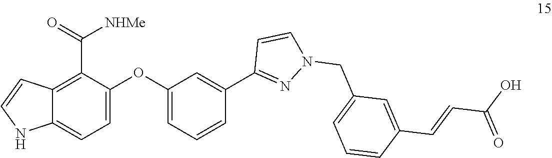 [SMILES: CNC(=O)c1c(Oc2cccc(-c3ccn(Cc4cccc(/C=C/C(=O)O)c4)n3)c2)ccc2[nH]ccc12]

### A. 5-(3-(1-(3-Iodobenzyl)-1H-pyrazol-3-yl)phenoxy)-N-methyl-1-tosyl-1H-indole-4-carboxamide

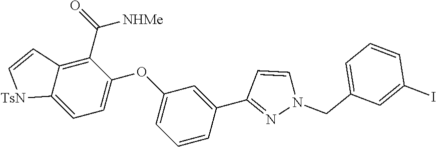 [SMILES: CNC(=O)c1c(Oc2cccc(-c3ccn(Cc4cccc(I)c4)n3)c2)ccc2c1ccn2S(=O)(=O)c1ccc(C)cc1]

A mixture of methyl 5-(3-(1-(3-iodobenzyl)-1H-pyrazol-3-yl)phenoxy)-1-tosyl-1H-indole-4-carboxylate (Intermediate 31, 0.5 g, 710 μmol) in 7 N methylamine in methanol (20 mL) was stirred for 15 days. The solvent was evaporated and the residue was purified by flash chromatography over silica (0-100% ethyl acetate in heptane) to give the title compound as a white solid (0.26 g, 52%).

### B. Methyl (E)-3-(3-((3-(3-((4-(methylcarbamoyl)-1-tosyl-1H-indol-5-yl)oxy)phenyl)-1H-pyrazol-1-yl)methyl)phenyl)acrylate

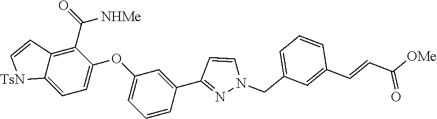 [SMILES: CNC(=O)c1c(Oc2cccc(-c3ccn(Cc4cccc(/C=C/C(=O)OC)c4)n3)c2)ccc2c1ccn2S(=O)(=O)c1ccc(C)cc1]

A mixture of triethylamine (516 μl, 3.70 mmol), 5-(3-(1-(3-iodobenzyl)-1H-pyrazol-3-yl)phenoxy)-N-methyl-1-tosyl-1H-indole-4-carboxamide (0.26 g, 370 μmol) in 1.5 mL of DMF was degassed for 10 minutes. Then palladium (II) acetate (4 mg, 18 μmol) and tri-o-tolylphosphine (22 mg, 74 μmol) were added and the 2 mL microwave vial was sealed. Methyl acrylate (44 μl, 481 μmol) was added and the mixture was heated at 100° C. for 35 minutes. The solvent was evaporated and the residue was purified by flash chromatography over silica (0-100% ethyl acetate in heptane) to give the title compound as a white solid (160 mg, 65%). LC-MS Retention time: 1.45 minutes. MS: 661.

### C. Example 15, (E)-3-(3-((3-(3-((4-(Methylcarbamoyl)-1H-indol-5-yl)oxy)phenyl)-1H-pyrazol-1-yl)methyl)phenyl)acrylic acid

A mixture of methyl (E)-3-(3-((3-(3-((4-(methylcarbamoyl)-1-tosyl-1H-indol-5-yl)oxy)phenyl)-1H-pyrazol-1-yl)methyl)phenyl)acrylate (0.16 g, 242 μmol) and potassium carbonate (67 mg, 484 μmol) in methanol (5 mL) was heated at 135° C. for 10 minutes. The solvent was evaporated and the residue was dissolved in 5 mL of water and extracted with ethyl acetate (20 mL×3). The combined organic extracts were washed with water, brine, dried over sodium sulfate, filtered and concentrated. The resulting residue was purified by flash chromatography over silica (0-100% ethyl acetate in heptane) to give methyl (E)-3-(3-((3-(3-((4-(methylcarbamoyl)-1H-indol-5-yl)oxy)phenyl)-1H-pyrazol-1-yl)methyl)phenyl)acrylate (40 mg, 33%). The aqueous layer from the extraction was concentrated, the resulting residue was purified by preparative HPLC to afford the title compound as a white solid (14 mg, 12%). ¹H NMR (400 MHz, CD₃OD) δ 7.72-7.57 (m, 3H), 7.56-7.22 (m, 8H), 6.90-6.82 (m, 2H), 6.72-6.66 (m, 1H), 6.62 (d, J=2.2 Hz, 1H), 6.46 (d, J=16.0 Hz, 1H), 5.37 (s, 2H), 2.84 (s, 3H) ppm. LC-MS Retention time: 1.10 minutes. MS: 493 m/z [M+H]⁺.

### Example 16

### 3-(3-((3-(3-((4-(Methylcarbamoyl)-1H-indol-5-yl)oxy)phenyl)-1H-pyrazol-1-yl)methyl)phenyl)propanoic acid

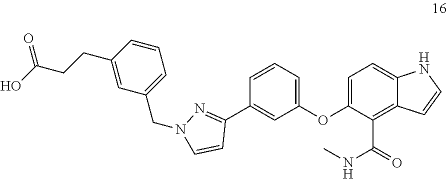 [SMILES: CNC(=O)c1c(Oc2cccc(-c3ccn(Cc4cccc(CCC(=O)O)c4)n3)c2)ccc2[nH]ccc12]

### A. Methyl 3-(3-((3-(3-((4-(methylcarbamoyl)-1H-indol-5-yl)oxy)phenyl)-1H-pyrazol-1-yl)methyl)phenyl)propanoate

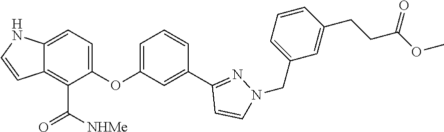 [SMILES: CNC(=O)c1c(Oc2cccc(-c3ccn(Cc4cccc(CCC(=O)OC)c4)n3)c2)ccc2[nH]ccc12]

A mixture of methyl (E)-3-(3-((3-(3-((4-(methylcarbamoyl)-1H-indol-5-yl)oxy)phenyl)-1H-pyrazol-1-yl)methyl)phenyl)acrylate (Example 15, 40 mg, 79 μmol,) and 10% Pd—C (16.81 mg, 16 μmol, 50% wet) in 10 mL of ethyl acetate was stirred under hydrogen overnight. The mixture was filtered through a pad of Celite and washed with ethyl acetate. The filtrate was concentrated and the resulting residue was purified by flash chromatography over silica (0-100% ethyl acetate in heptane) to give the title compound as a white solid (30 mg, 75%).

### B. Example 16, 3-(3-((3-(3-((4-(Methylcarbamoyl)-1H-indol-5-yl)oxy)phenyl)-1H-pyrazol-1-yl)methyl)phenyl)propanoic acid

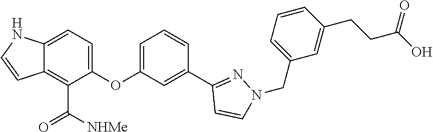 [SMILES: CNC(=O)c1c(Oc2cccc(-c3ccn(Cc4cccc(CCC(=O)O)c4)n3)c2)ccc2[nH]ccc12]

A mixture of methyl 3-(3-((3-(3-((4-(methylcarbamoyl)-1H-indol-5-yl)oxy)phenyl)-1H-pyrazol-1-yl)methyl)phenyl)propanoate (30 mg, 59 μmol) and lithium hydroxide (4 mg, 177 μmol) in 4 mL of water and 10 mL of THF was stirred overnight. The solvent was evaporated, the residue was dissolved in 2 mL of water and acidified with 1N HCl. The solid was collected by filtration and dried to afford the title compound as a clean white solid (12 mg, 41%). ¹H NMR (400 MHz, CD₃OD CD₃OD) δ 7.62 (d, J=2.4 Hz, 1H), 7.52-7.40 (m, 2H), 7.39-7.20 (m, 4H), 7.20-7.10 (m, 2H), 7.05 (dt, J=7.5, 1.5 Hz, 1H), 6.90-6.81 (m, 2H), 6.69 (dd, J=3.1, 0.9 Hz, 1H), 6.60 (d, J=2.4 Hz, 1H), 5.31 (s, 2H), 2.86 (m, 5H), 2.56 (t, J=7.7 Hz, 2H) ppm. LC-MS Retention time: 1.11 minutes. MS: 495 m/z [M+H]⁺.

### Example 17

### 3-(3-((3-(2-Fluoro-5-((6-fluoro-4-methyl-1H-indol-5-yl)oxy)phenyl)-1H-pyrazol-1-yl)methyl)phenyl)propanamide

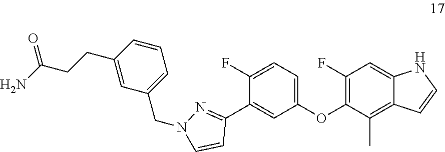 [SMILES: Cc1c(Oc2ccc(F)c(-c3ccn(Cc4cccc(CCC(N)=O)c4)n3)c2)c(F)cc2[nH]ccc12]

A mixture of 3-(3-((3-(2-fluoro-5-((6-fluoro-4-methyl-1H-indol-5-yl)oxy)phenyl)-1H-pyrazol-1-yl)methyl)phenyl)propanoic acid (Example 14, 70 mg, 0.14 mmol), ammonium chloride (23 mg, 0.43 mmol), HATU (87 mg, 0.23 mmol) and diisopropylethylamine (93 mg, 0.72 mmol) in DMF (20 mL) was stirred at room temperature for three hours. The mixture was diluted with water (30 mL) and extracted with ethyl acetate (40 mL×3). The combined organic extracts were washed with brine (20 mL×3), dried over sodium sulfate, filtered and concentrated. The resulting residue was purified by preparative HPLC to afford the title compound as a white solid (35 mg, 46%). ¹H NMR (400 MHz, CD₃OD) δ 7.65 (d, J=2.4 Hz, 1H), 7.39-7.37 (m, 1H), 7.28-7.06 (m, 7H), 6.79-6.76 (m, 1H), 6.69-6.68 (m, 1H), 6.52 (d, J=3.2 Hz, 1H), 5.32 (s, 2H), 2.89 (t, J=7.6 Hz, 2H), 2.47 (t, J=7.6 Hz, 2H), 2.39 (s, 3H) ppm. LC-MS Retention time: 1.35 min; m/z: 487 m/z [M+H]⁺.

### Example 18

### 3-(5-((3-(2-Fluoro-5-((6-fluoro-4-methyl-1H-indol-5-yl)oxy)phenyl)-1H-pyrazol-1-yl)methyl)thiazol-2-yl)propanoic acid

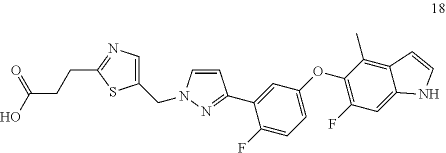 [SMILES: Cc1c(Oc2ccc(F)c(-c3ccn(Cc4cnc(CCC(=O)O)s4)n3)c2)c(F)cc2[nH]ccc12]

Exchanging 5-(3-(1H-pyrazol-3-yl)phenoxy)-4-methyl-1-tosyl-1H-indole for 6-fluoro-5-(4-fluoro-3-(1H-pyrazol-3-yl)phenoxy)-4-methyl-1-tosyl-1H-indole (Intermediate 30-2) and ethyl 3-(3-(bromomethyl)phenyl)propanoate for ethyl 3-(5-(chloromethyl)thiazol-2-yl)propanoate, the procedure described for Example 1 was used to prepare the title compound (3.8 mg) as a white solid). ¹H NMR (400 MHz, DMSO-d₆). δ 12.29 (s, 1H), 11.45 (s, 1H), 7.87 (d, 1H), 7.62 (s, 1H), 7.39 (t, 1H), 7.23-7.29 (m, 3H), 6.82-6.87 (m, 1H), 6.60 (t, 1H), 6.53 (s, 1H), 5.55 (s, 2H), 3.10 (t, 2H), 2.64 (t, 2H), 2.34 (s, 3H) ppm. LC-MS Retention time: 1.30 min; MS: 495[M+H]⁺.

### Examples 19 and 20

### 3-(3-((4-Fluoro-3-(3-((6-fluoro-4-methyl-1H-indol-5-yl)oxy)phenyl)-1H-pyrazol-1-yl)methyl)phenyl)propanoic acid

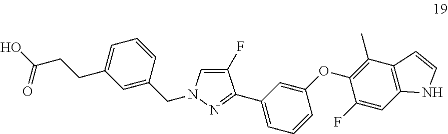 [SMILES: Cc1c(Oc2cccc(-c3nn(Cc4cccc(CCC(=O)O)c4)cc3F)c2)c(F)cc2[nH]ccc12]

### 3-(3-((4-Fluoro-5-(3-((6-fluoro-4-methyl-1H-indol-5-yl)oxy)phenyl)-1H-pyrazol-1-yl)methyl)phenyl)propanoic acid

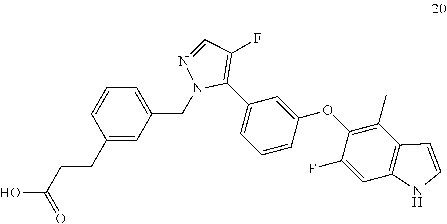 [SMILES: Cc1c(Oc2cccc(-c3c(F)cnn3Cc3cccc(CCC(=O)O)c3)c2)c(F)cc2[nH]ccc12]

### A. 4-Fluoro-N,N-dimethyl-1H-pyrazole-1-sulfonamide

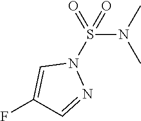

To a solution of 4-fluoro-1H-pyrazole (1.72 g, 20 mmol) in THF (20 mL) was added sodium hydride (1.2 g, 30 mmol) at 0° C. and stirred at 0° C. for 0.5 hour. Dimethylsulfamoyl chloride (4.29 g, 30 mmol) was added dropwise at 0° C. and stirred at room temperature overnight. The reaction mixture was diluted with ethyl acetate (100 mL), washed with brine (30 mL×3), dried with sodium sulfate, filtered and concentrated. The resulting residue was purified by flash chromatography over silica (petroleum ether/ethyl acetate, v/v, 3/1) to afford 4-fluoro-N,N-dimethyl-1H-pyrazole-1-sulfonamide (3.5 g, 90%). MS: 194 m/z [M+H]⁺.

### B. 3-Bromo-4-fluoro-N,N-dimethyl-1H-pyrazole-1-sulfonamide

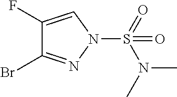

To a solution of 4-fluoro-N,N-dimethyl-1H-pyrazole-1-sulfonamide (3.5 g, 18.1 mmol) in THF (50 mL) was added n-butyl lithium (2.5N in THF, 8 mL, 20 mmol) at −78° C. and stirred for 0.5 hour. 1,2-dibromo-1,1,2,2-tetrachloroethane (6.42 g, 19.9 mmol) was added dropwise at −78° C., stirred for another four hours at −78°, quenched with saturated ammonium chloride (50 mL) and extracted with ethyl acetate (50 mL×3). The combined organic extracts were washed with brine (30 mL×3), dried with sodium sulfate, filtered and concentrated. The resulting residue was purified by flash chromatography over silica (petroleum ether/ethyl acetate, v/v, 3/1) to afford the title compound (4.05 g, 82%). MS: 272, 274 m/z [M+H]⁺.

### C. 3-Bromo-4-fluoro-1H-pyrazole

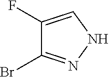

To a solution of 3-bromo-4-fluoro-N,N-dimethyl-1H-pyrazole-1-sulfonamide (4.05 g, 14.9 mmol) in dichloromethane (50 mL) was added trifluoroacetic acid (3 mL) at 0° C. and stirred at room temperature overnight. The reaction solution was diluted with dichloromethane (50 mL), washed with aqueous saturated sodium carbonate (30 mL×2) and water (30 mL), dried with sodium sulfate, filtered and concentrated to give the title compound as a yellow oil (3.9 g, crude).

### D. Ethyl 3-(3-((3-bromo-4-fluoro-1H-pyrazol-1-yl)methyl)phenyl)propanoate and ethyl 3-(3-((5-bromo-4-fluoro-1H-pyrazol-1-yl)methyl)phenyl)propanoate

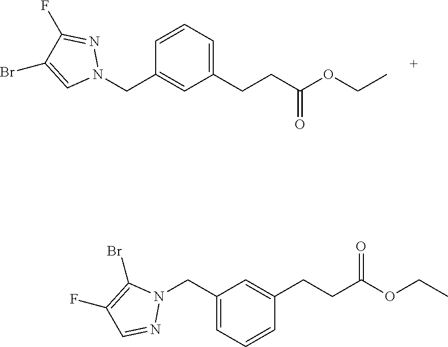 [SMILES: CCOC(=O)CCc1cccc(Cn2cc(Br)c(F)n2)c1 ; CCOC(=O)CCc1cccc(Cn2ncc(F)c2Br)c1]

To a solution of 3-bromo-4-fluoro-1H-pyrazole (300 mg, crude) and ethyl 3-(3-(bromomethyl)phenyl)propanoate (309 mg, 1.14 mmol) in DMF (5 mL) was added potassium carbonate (315 mg, 2.28 mmol) at 0° C. and stirred at room temperature overnight. The reaction mixture was diluted with ethyl acetate (30 mL), washed with brine (10 mL×3), dried with sodium sulfate, filtered and concentrated. The resulting residue was purified by flash chromatography over silica (petroleum ether/ethyl acetate, v/v, 3/1-2/1) to afford an inseparable mixture of ethyl 3-(3-((3-bromo-4-fluoro-1H-pyrazol-1-yl)methyl)phenyl)propanoate and ethyl 3-(3-((5-bromo-4-fluoro-1H-pyrazol-1-yl)methyl)phenyl)propanoate (300 mg, 54% of 2 steps). MS: 355, 357 m/z [M+H]⁺.

### E. Ethyl 3-(3-((4-fluoro-3-(3-((6-fluoro-4-methyl-1H-indol-5-yl)oxy)phenyl)-1H-pyrazol-1-yl)methyl)phenyl)propanoate and

### Ethyl 3-(3-((4-fluoro-5-(3-((6-fluoro-4-methyl-1H-indol-5-yl)oxy)phenyl)-1H-pyrazol-1-yl)methyl)phenyl)propanoate

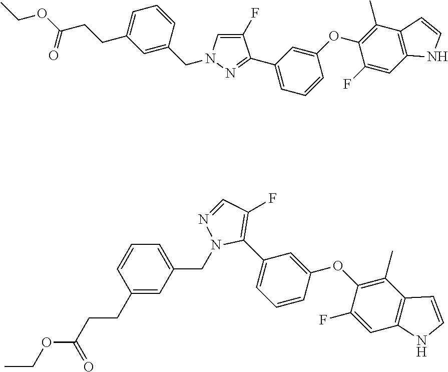 [SMILES: CCOC(=O)CCc1cccc(Cn2cc(F)c(-c3cccc(Oc4c(F)cc5[nH]ccc5c4C)c3)n2)c1 ; CCOC(=O)CCc1cccc(Cn2ncc(F)c2-c2cccc(Oc3c(F)cc4[nH]ccc4c3C)c2)c1]

To a mixture of an inseparable mixture of ethyl 3-(3-((3-bromo-4-fluoro-1H-pyrazol-1-yl)methyl)phenyl)propanoate and ethyl 3-(3-((5-bromo-4-fluoro-1H-pyrazol-1-yl)methyl)phenyl)propanoate (300 mg, 0.85 mmol), 6-fluoro-4-methyl-5-(3-(4,4,5,5-tetramethyl-1,3,2-dioxaborolan-2-yl)phenoxy)-1H-indole (Intermediate 32, 373 mg, 1.02 mmol) and potassium carbonate (234 mg, 1.69 mmol) in dioxane/water (v/v, 8/2 mL) was added Pd(dppf)Cl₂ (69 mg, 0.08 mmol) and heated at 120° C. for two hours in a nitrogen filled 20 mL microwave tube in a microwave synthesizer. The reaction mixture was diluted with ethyl acetate (100 mL), washed with water (30 mL) and brine, dried with sodium sulfate, filtered and concentrated. The resulting residue was purified by flash chromatography over silica (petroleum ether/ethyl acetate/dichloromethane, v/v, 2/1/1) to afford mixtures of ethyl 3-(3-((4-fluoro-3-(3-((6-fluoro-4-methyl-1H-indol-5-yl)oxy)phenyl)-1H-pyrazol-1-yl)methyl)phenyl)propanoate and ethyl 3-(3-((4-fluoro-5-(3-((6-fluoro-4-methyl-1H-indol-5-yl)oxy)phenyl)-1H-pyrazol-1-yl)methyl)phenyl)propanoate as a yellow solid (100 mg, 23%). MS: 516 m/z [M+H]⁺ and 516 m/z [M+H]⁺.

### F. Example 19, 3-(3-((4-Fluoro-3-(3-((6-fluoro-4-methyl-1H-indol-5-yl)oxy)phenyl)-1H-pyrazol-1-yl)methyl)phenyl)propanoic acid and

### Example 20, 3-(3-((4-Fluoro-5-(3-((6-fluoro-4-methyl-1H-indol-5-yl)oxy)phenyl)-1H-pyrazol-1-yl)methyl)phenyl)propanoic acid

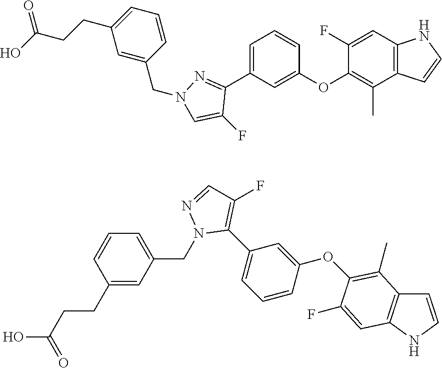 [SMILES: Cc1c(Oc2cccc(-c3nn(Cc4cccc(CCC(=O)O)c4)cc3F)c2)c(F)cc2[nH]ccc12 ; Cc1c(Oc2cccc(-c3c(F)cnn3Cc3cccc(CCC(=O)O)c3)c2)c(F)cc2[nH]ccc12]

To a solution of ethyl 3-(3-((4-fluoro-3-(3-((6-fluoro-4-methyl-1H-indol-5-yl)oxy)phenyl)-1H-pyrazol-1-yl)methyl)phenyl)propanoate and ethyl 3-(3-((4-fluoro-5-(3-((6-fluoro-4-methyl-1H-indol-5-yl)oxy)phenyl)-1H-pyrazol-1-yl)methyl)phenyl)propanoate (100 mg, 0.19 mmol) in THF (5 mL) was added a solution of lithium hydroxide (28 mg, 1.17 mmol) in water (1 mL) and stirred at room temperature overnight. The solvent was evaporated and the residue was dissolved in 2 mL of water, acidified with 1 N hydrochloric acid (pH˜2) and concentrated to dryness. The resulting residue was purified by preparative HPLC to give Example 19, 3-(3-((4-fluoro-3-(3-((6-fluoro-4-methyl-1H-indol-5-yl)oxy)phenyl)-1H-pyrazol-1-yl)methyl)phenyl)propanoic acid (46 mg, 49%) and Example 20, 3-(3-((4-fluoro-5-(3-((6-fluoro-4-methyl-1H-indol-5-yl)oxy)phenyl)-1H-pyrazol-1-yl)methyl)phenyl)propanoic acid, both as a white solid (11 mg, 12%).

Example 19, 3-(3-((4-fluoro-3-(3-((6-fluoro-4-methyl-1H-indol-5-yl)oxy)phenyl)-1H-pyrazol-1-yl)methyl)phenyl)propanoic acid: ¹H NMR (400 MHz, CD₃OD) δ 7.62 (d, J=4.8 Hz, 1H), 7.44 (d, J=7.2 Hz, 1H), 7.34-7.05 (m, 8H), 6.79 (dd, J=8.0, 2.0 Hz, 1H), 6.52 (dd, J=3.2, 0.8 Hz, 1H), 5.21 (s, 2H), 2.90 (t, J=7.6 Hz, 2H), 2.57 (t, J=7.6 Hz, 2H), 2.38 (s, 3H) ppm. MS: 488 m/z [M+H]⁺.

Example 20, 3-(3-((4-fluoro-5-(3-((6-fluoro-4-methyl-1H-indol-5-yl)oxy)phenyl)-1H-pyrazol-1-yl)methyl)phenyl)propanoic acid: ¹H NMR (400 MHz, CD₃OD) δ 7.52 (d, J=4.4 Hz, 1H), 7.40 (dd, J=8.0, 8.0 Hz, 1H), 7.27 (d, J=3.2 Hz, 1H), 7.10-6.94 (m, 5H), 6.72-6.67 (m, 3H), 6.48 (dd, J=3.2, 0.8 Hz, 1H), 5.22 (s, 2H), 2.75 (t, J=7.6 Hz, 2H), 2.45 (t, J=7.6 Hz, 2H), 2.30 (s, 3H) ppm. MS: 488 m/z [M+H]⁺.

### Example 21

### 3-(3-((5-(3-((6-Fluoro-4-methyl-1H-indol-5-yl)oxy)phenyl)-1H-pyrrol-2-yl)methyl)phenyl)propanoic acid

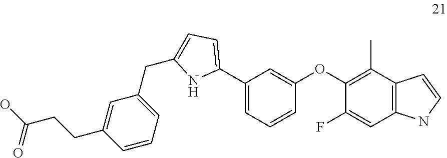 [SMILES: Cc1c(Oc2cccc(-c3ccc(Cc4cccc(CCC(=O)O)c4)[nH]3)c2)c(F)cc2[nH]ccc12]

### A. tert-butyl 2-(3-bromobenzyl)-1H-pyrrole-1-carboxylate

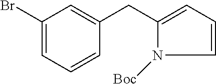 [SMILES: CC(C)(C)OC(=O)n1cccc1Cc1cccc(Br)c1]

To a solution of methyl 3-(3-bromophenyl)-2-((tert-butoxycarbonyl)amino)propanoate (3.75 g, 10.5 mmol) in dichloromethane (50 mL) was added DIBAL-H (1N in toluene, 21 mL, 21 mmol) drop-wise at −78° C. and stirred at this temperature for two hours. The reaction was quenched with aqueous saturated ammonium chloride (100 mL) and extracted with dichloromethane (50 mL×3). The combined organic extracts were washed with brine (100 mL), dried with sodium sulfate, filtered and concentrated to give tert-butyl (1-(3-bromophenyl)-3-oxopropan-2-yl)carbamates a yellow solid (3.5 g, crude). This solid was dissolved in toluene (20 mL), 2-(triphenyl-15-phosphanylidene)acetaldehyde (3.35 g, 11 mmol) was added and stirred at 60° C. overnight. The reaction mixture was cooled to room temperature, diluted with ethyl acetate (100 mL), washed with water (30 mL×2) and brine (30 mL×1), dried with sodium sulfate, filtered and concentrated to give tert-butyl (Z)-(1-(3-bromophenyl)-5-oxopent-3-en-2-yl)carbamate as a yellow solid (4.5 g, crude). The yellow solid was dissolved in dichloromethane (20 mL), hydrochloric acid (3 N in dioxane, 10 mL, 30.0 mmol) was added and stirred at room temperature overnight. The resulting solution was diluted with dichloromethane (100 mL), washed with water (30 mL×2) and brine (30 mL×1), dried over sodium sulfate, filtered and concentrated. The resulting residue was purified by silica gel column chromatography (petroleum ether/ethyl acetate, v/v, 8/1-3/1) to afford the title compound as a white solid (1.2 g, 34%, 3 steps). MS: 336 m/z [M+H]⁺.

### B. Tert-butyl (E)-2-(3-(3-ethoxy-3-oxoprop-1-en-1-yl)benzyl)-1H-pyrrole-1-carboxylate

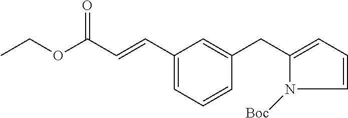 [SMILES: CCOC(=O)/C=C/c1cccc(Cc2cccn2C(=O)OC(C)(C)C)c1]

To a solution of tert-butyl 2-(3-bromobenzyl)-1H-pyrrole-1-carboxylate (1.3 g, 3.88 mmol) in DMF (10 mL) were added Pd(OAc)₂ (87 mg, 0.39 mmol), triethylamine (3.92 g, 38.8 mmol), tri-O-tolylposphine (236 mg, 0.77 mmol) and ethyl acrylate (582 mg, 5.82 mmol) and heated at 110° C. for two hours in a nitrogen filled microwave tube in a microwave synthesizer. The reaction mixture was diluted with ethyl acetate (100 mL), washed with brine (20 mL×3), dried with sodium sulfate, filtered and concentrated. The resulting residue was purified by flash chromatography over silica (petroleum ether/ethyl acetate, v/v, 3/1) to afford the title compound as a pale solid (690 mg, 51%). MS: 356 m/z [M+H]⁺.

### C. Tert-butyl 2-(3-(3-ethoxy-3-oxopropyl)benzyl)-1H-pyrrole-1-carboxylate

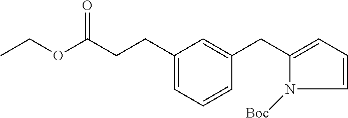 [SMILES: CCOC(=O)CCc1cccc(Cc2cccn2C(=O)OC(C)(C)C)c1]

To a solution of tert-butyl (E)-2-(3-(3-ethoxy-3-oxoprop-1-en-1-yl)benzyl)-1H-pyrrole-1-carboxylate (690 mg, 1.94 mmol) in ethanol (10 mL) was added Pd/C (100 mg, 10% Pd on carbon). The resulting mixture was stirred at room temperature for one hour under hydrogen atmosphere and filtered through a pad of celite. The filtrate was concentrated to give the title compound as a light yellow solid (600 mg, 86%), which was used directly in the next step. MS: 358 m/z [M+H]⁺.

### D. Ethyl 3-(3-((1H-pyrrol-2-yl)methyl)phenyl)propanoate

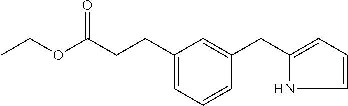 [SMILES: CCOC(=O)CCc1cccc(Cc2ccc[nH]2)c1]

To a solution of tert-butyl 2-(3-(3-ethoxy-3-oxopropyl)benzyl)-1H-pyrrole-1-carboxylate (550 mg, 1.54 mmol) in dichloromethane (10 mL) was added trifluoroacetic acid (1.5 mL) and then stirred at room temperature overnight. The reaction solution was diluted with dichloromethane (50 mL), washed with aqueous saturated sodium carbonate (30 mL×2), water (30 mL), dried over sodium sulfate, filtered and concentrated to give the title compound as a yellow oil (340 mg, 81%). MS: 258 m/z [M+H]⁺.

### E. Ethyl 3-(3-((5-bromo-1H-pyrrol-2-yl)methyl)phenyl)propanoate

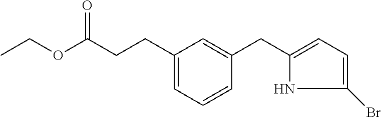 [SMILES: CCOC(=O)CCc1cccc(Cc2ccc(Br)[nH]2)c1]

To a solution of ethyl 3-(3-((1H-pyrrol-2-yl)methyl)phenyl)propanoate (340 mg, 1.32 mmol) in THF (10 mL) was added N-bromosuccinimide (259 mg, 1.46 mmol) at −78° C. and then stirred at this temperature for one hour. The resulting solution was diluted with ethyl acetate (50 mL), washed with saturated aqueous sodium carbonate (30 mL×2), water (30 mL), dried over sodium sulfate, filtered and concentrated. The resulting residue was purified by flash chromatography over silica (petroleum ether/ethyl acetate, v/v, 1/1) to afford the title compound as a pale solid (130 mg, 29%). MS: 336 m/z [M+H]⁺.

### F. Ethyl 3-(3-((5-(3-((6-fluoro-4-methyl-1H-indol-5-yl)oxy)phenyl)-1H-pyrrol-2-yl)methyl)phenyl)propanoate

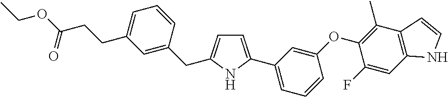 [SMILES: CCOC(=O)CCc1cccc(Cc2ccc(-c3cccc(Oc4c(F)cc5[nH]ccc5c4C)c3)[nH]2)c1]

To a mixture of 6-fluoro-4-methyl-5-(3-(4,4,5,5-tetramethyl-1,3,2-dioxaborolan-2-yl)phenoxy)-1H-indole (Intermediate 32, 158 mg, 0.43 mmol), ethyl 3-(3-((5-bromo-1H-pyrrol-2-yl)methyl)phenyl)propanoate (120 mg, 0.36 mmol) and sodium carbonate (76 mg, 0.71 mmol) in dioxane/water (8+2 mL) was added Pd(dppf)Cl₂ (30 mg, 0.04 mmol) and heated at 120° C. for two hours in a nitrogen filled microwave tube in a Biotage microwave synthesizer. The reaction mixture was concentrated and the resulting residue was purified by flash chromatography over silica (petroleum ether/ethyl acetate/dichloromethane, v/v, 2/1/1) to afford the title compound as a pale solid (73 mg, 41%). MS: 497 m/z [M+H]⁺.

### G. Example 21, 3-(3-((5-(3-((6-Fluoro-4-methyl-1H-indol-5-yl)oxy)phenyl)-1H-pyrrol-2-yl)methyl)phenyl)propanoic acid

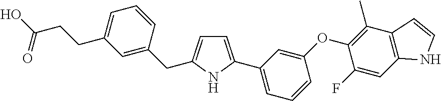 [SMILES: Cc1c(Oc2cccc(-c3ccc(Cc4cccc(CCC(=O)O)c4)[nH]3)c2)c(F)cc2[nH]ccc12]

To a solution of ethyl 3-(3-((5-(3-((6-fluoro-4-methyl-1H-indol-5-yl)oxy)phenyl)-1H-pyrrol-2-yl)methyl)phenyl)propanoate (73 mg, 0.15 mmol) in THF (4 mL) was added a solution of lithium hydroxide (21 mg, 0.88 mmol) in water (1 mL) and stirred at room temperature overnight. The reaction mixture was diluted with water (10 mL). THF was removed by evaporation, the resulting aqueous phase was acidified with 1 N hydrochloric acid and extracted with ethyl acetate (30 mL×3). The combined organic extracts were washed with brine, dried over sodium sulfate, concentrated and the resulting residue was purified by preparative HPLC to give the title compound as a white solid (17 mg, 25%). MS: 469 m/z [M+H]⁺. ¹H NMR (400 MHz, CD₃OD) δ 7.35-6.93 (m, 11H), 6.76 (d, J=8.8 Hz, 1H), 6.49 (s, 1H), 3.91 (s, 2H), 2.86 (t, J=7.6 Hz, 2H), 2.55 (t, J=7.6 Hz, 2H), 2.32 (s, 3H) ppm.

### Example 22

### 3-(3-((2-(2-Fluoro-5-((6-fluoro-4-(methylsulfonyl)-1H-indol-5-yl)oxy)phenyl)-1H-imidazol-5-yl)methyl)phenyl)propanoic acid

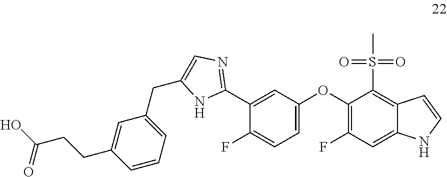 [SMILES: CS(=O)(=O)c1c(Oc2ccc(F)c(-c3ncc(Cc4cccc(CCC(=O)O)c4)[nH]3)c2)c(F)cc2[nH]ccc12]

### A. Methyl 3-(3-((2-(2-fluoro-5-((6-fluoro-4-(methylthio)-1H-indol-5-yl)oxy)phenyl)-1H-imidazol-5-yl)methyl)phenyl)propanoate

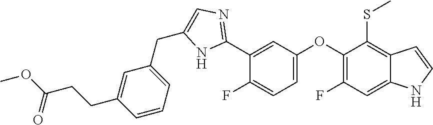 [SMILES: COC(=O)CCc1cccc(Cc2cnc(-c3cc(Oc4c(F)cc5[nH]ccc5c4SC)ccc3F)[nH]2)c1]

To a solution of 2-fluoro-5-((6-fluoro-4-(methylthio)-1H-indol-5-yl)oxy)benzimidamide (Intermediate 24-4, 60 mg, 0.18 mmol) and methyl 3-(3-(3-bromo-2-oxopropyl)phenyl)propanoate (Intermediate 21, 66 mg, 0.22 mmol) in DMF (3 mL) was added potassium carbonate (50 mg, 0.36 mmol). The reaction mixture was stirred at 75° C. for five hours, quenched with water (50 mL) and extracted with ethyl acetate (20 mL×3). The combined organic extracts were washed with brine (20 mL), dried over sodium sulfate, filtered and concentrated. The resulting residue was purified by flash chromatography over silica (0-100% ethyl acetate in petroleum ether) to give the title compound as a yellow solid (50 mg, 52%). MS: 534 m/z [M+H]⁺.

### B. Methyl 3-(3-((2-(2-fluoro-5-((6-fluoro-4-(methylsulfonyl)-1H-indol-5-yl)oxy)phenyl)-1H-imidazol-5-yl)methyl)phenyl)propanoate

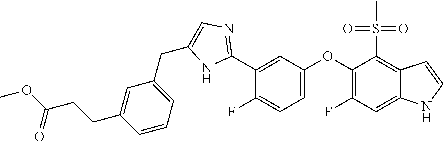 [SMILES: COC(=O)CCc1cccc(Cc2cnc(-c3cc(Oc4c(F)cc5[nH]ccc5c4S(C)(=O)=O)ccc3F)[nH]2)c1]

To a solution of methyl 3-(3-((2-(2-fluoro-5-((6-fluoro-4-(methylthio)-1H-indol-5-yl)oxy)phenyl)-1H-imidazol-5-yl)methyl)phenyl)propanoate (50 mg, 0.094 mmol) in methanol (5 mL) was added a solution of ammonium molybdate tetrahydrate (100 mg) in 0.5 mL of hydrogen peroxide (30% in water) and stirred at room temperature for 2.5 hours. The reaction mixture diluted with ethyl acetate (30 mL), washed with water (10 mL×3), saturated sodium sulfite (10 mL) and brine (10 mL×2), dried over sodium sulfate and concentrated to give the title compound as a yellow solid (35 mg, 66%). MS: 566 m/z [M+H]⁺.

### C. Example 22, 3-(3-((2-(2-Fluoro-5-((6-fluoro-4-(methylsulfonyl)-1H-indol-5-yl)oxy)phenyl)-1H-imidazol-5-yl)methyl)phenyl)propanoic acid

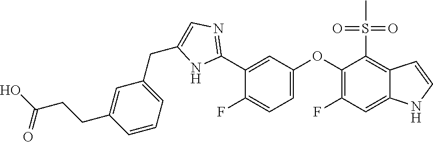 [SMILES: CS(=O)(=O)c1c(Oc2ccc(F)c(-c3ncc(Cc4cccc(CCC(=O)O)c4)[nH]3)c2)c(F)cc2[nH]ccc12]

To a solution of methyl 3-(3-((2-(2-fluoro-5-((6-fluoro-4-(methylsulfonyl)-1H-indol-5-yl)oxy)phenyl)-1H-imidazol-5-yl)methyl)phenyl)propanoate (35 mg, 0.062 mmol) in methanol (1 mL) and THF (3 mL) was added lithium hydroxide (1M in water, 1 mL) and stirred at room temperature for two hours. The reaction mixture was acidified with 1 N hydrochloric acid to pH 4, diluted with ethyl acetate (50 mL), washed with brine (10 mL×3), dried over sodium sulfate, filtered and concentrated. The resulting residue was purified by preparative HPLC to afford the title compound as a yellow solid (30 mg, 88%). MS: 552 m/z [M+H]⁺. ¹H NMR (400 MHz, CD₃OD) δ 7.67 (d, J=10.6 Hz, 1H), 7.55-7.49 (m, 2H), 7.25 (d, J=10.9 Hz, 1H), 7.23-7.19 (m, 2H), 7.15 (s, 1H), 7.10 (s, 2H), 7.03 (d, J=8.8 Hz, 1H), 6.84 (s, 1H), 3.95 (s, 2H), 3.50 (s, 3H), 2.89 (t, J=7.6 Hz, 2H), 2.58 (t, J=7.7 Hz, 2H) ppm.

### Example 23

### Enantiomer 1 of 3-(2-fluoro-3-(1-(1-(3-((6-fluoro-4-(methylsulfonyl)-1H-indol-5-yl)oxy)phenyl)-1H-pyrazol-3-yl)ethyl)phenyl)propanoic acid (23A)

### And

### Example

### Enantiomer 2 of 3-(2-fluoro-3-(1-(1-(3-((6-fluoro-4-(methylsulfonyl)-1H-indol-5-yl)oxy)phenyl)-1H-pyrazol-3-yl)ethyl)phenyl)propanoic acid (23B)

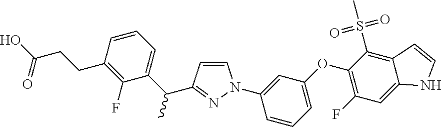 [SMILES: CC(c1ccn(-c2cccc(Oc3c(F)cc4[nH]ccc4c3S(C)(=O)=O)c2)n1)c1cccc(CCC(=O)O)c1F]

### A. 2-(3-Bromo-2-fluorophenyl)-N-methoxy-N-methylpropanamide

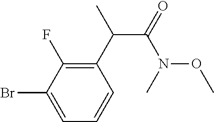 [SMILES: CON(C)C(=O)C(C)c1cccc(Br)c1F]

To a solution of 2-(3-bromo-2-fluorophenyl)propanoic acid (5.0 g, 20.2 mmol) in THF (50 mL) were added N,O-dimethylhydroxylamine hydrochloride (2.96 g, 30.4 mmol), HATU (9.23 g, 24.3 mmol) and triethylamine (6.13 g, 60.7 mmol). The mixture was stirred at room temperature overnight, quenched with saturated sodium carbonate and extracted with ethyl acetate. The combined organic extracts were washed with brine, dried over sodium sulfate, filtered and concentrated. The crude production was purified by flash silica gel column chromatography (20% ethyl acetate in petroleum ether) to give the title compound as a yellow oil (4.6 g, yield 78%). MS: 290.0 m/z [M+H]⁺. ¹H NMR (400 MHz, CDCl₃) δ 7.44-7.40 (m, 1H), 7.32-7.28 (m, 1H), 7.01-6.97 (m, 1H), 4.47-4.45 (m, 1H), 3.53 (s, 3H), 3.16 (s, 3H), 1.44-1.42 (m, 3H), ppm.

### B. 3-(3-Bromo-2-fluorophenyl)butan-2-one

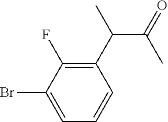

To a solution of 2-(3-bromo-2-fluorophenyl)-N-methoxy-N-methylpropanamide (4.6 g, 15.9 mmol, 1.0 eq.) in THF (50 mL) was added methylmagnesium bromide (26.4 mL, 79.3 mmol). The mixture was stirred at room temperature overnight, quenched with saturated ammonium chloride and extracted with ethyl acetate. The combined organic extracts were washed with brine, dried over sodium sulfate, filtered and concentrated. The residue was purified by silica gel column chromatography (20% ethyl acetate in petroleum ether) to give the title compound as a yellow oil (3.6 g, 93%). MS: 245 m/z [M+H]⁺. ¹H NMR (400 MHz, CDCl₃) δ 7.49-7.45 (m, 1H), 7.13-7.10 (m, 1H), 7.03-6.99 (m, 1H), 4.11-4.06 (m, 1H), 2.11 (s, 3H), 1.40 (d, J=6.8 Hz, 3H) ppm.

### C. Ethyl 3-(2-fluoro-3-(3-oxobutan-2-yl)phenyl)propanoate

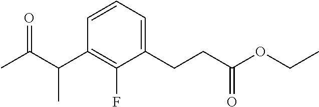 [SMILES: CCOC(=O)CCc1cccc(C(C)C(C)=O)c1F]

Exchanging 3-Iodophenylacetic acid for 3-(3-Bromo-2-fluorophenyl)butan-2-one, the two step reaction sequence described for Intermediate 21A and 21B was used to prepare the title compound as a yellow oil. MS: 267 m/z [M+H]⁺.

### D. Ethyl 3-(3-(1-(1H-pyrazol-3-yl)ethyl)-2-fluorophenyl)propanoate

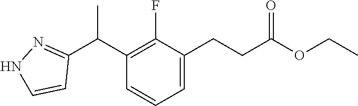 [SMILES: CCOC(=O)CCc1cccc(C(C)c2cc[nH]n2)c1F]

To a solution of ethyl 3-(2-fluoro-3-(3-oxobutan-2-yl)phenyl)propanoate (1.0 g, 3.76 mmol) in DMF (5 mL) was added DMF-DMA (2.24 g, 18.8 mmol). The mixture was stirred at 120° C. for two hours and concentrated. The residue was purified by flash chromatography over silica gel (50% ethyl acetate in petroleum ether) to afford ethyl (E)-3-(3-(5-(dimethylamino)-3-oxopent-4-en-2-yl)-2-fluorophenyl)propanoate as a yellow oil (1.0 g, 83%). ¹H NMR (400 MHz, CD₃OD) δ 7.65 (d, J=8.0 Hz, 1H), 7.22-7.03 (m, 3H), 5.06 (d, J=12.4 Hz, 1H), 4.13-3.65 (m, 3H), 3.11-2.08 (m, 8H), 2.64-2.60 (m, 2H), 2.14-2.20 (m, 6H) ppm.

To a solution of the above ethyl (E)-3-(3-(5-(dimethylamino)-3-oxopent-4-en-2-yl)-2-fluorophenyl)propanoate (1.0 g, 3.11 mmol) in ethanol (10 mL) was added hydrazine monohydrate (311 mg, 6.22 mmol). The mixture was refluxed for five hours and concentrated. The residue was purified by flash chromatography over silica gel (20% ethyl acetate in petroleum ether) to give the title compound (700 mg, 77%) as a yellow oil. ¹H NMR (400 MHz, CDCl₃) δ 7.48 (d, J=2.0 Hz, 1H), 7.09-6.97 (m, 3H), 6.15 (d, J=2.0 Hz, 1H), 4.54 (q, J=7.2 Hz, 1H), 4.17 (q, J=6.8 Hz, 2H), 2.97 (q, J=7.6 Hz, 2H), 2.62 (q, J=8.0 Hz, 2H), 1.64 (d, J=7.6 Hz, 3H), 1.23 (t, J=7.2 Hz, 3H) ppm.

### E. Ethyl 3-(2-fluoro-3-(1-(1-(3-((6-fluoro-4-(methylthio)-1-tosyl-1H-indol-5-yl)oxy)phenyl)-1H-pyrazol-3-yl)ethyl)phenyl)propanoate

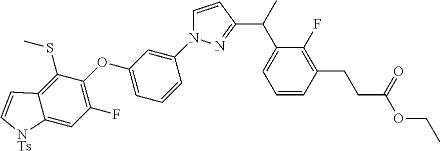 [SMILES: CCOC(=O)CCc1cccc(C(C)c2ccn(-c3cccc(Oc4c(F)cc5c(ccn5S(=O)(=O)c5ccc(C)cc5)c4SC)c3)n2)c1F]

To a nitrogen degassed solution of ethyl 3-(3-(1-(1H-pyrazol-3-yl)ethyl)-2-fluorophenyl)propanoate (460 mg, 1.58 mmol), CuI (50 mg, 0.26 mmol), N,N′-Dimethyl-1,2-ethanediamine (46 mg, 0.53 mmol) in toluene (10 mL) were added potassium carbonate (364 mg, 2.64 mmol) and 6-fluoro-5-(3-iodophenoxy)-4-(methylthio)-1-tosyl-1H-indole (Intermediate 45, 730 mg, 1.32 mmol). The mixture was stirred at 150° C. in a microwave reactor for four hours and concentrated. The residue was purified by flash silica gel column chromatography (25% ethyl acetate in petroleum ether) to give the title compound as a white solid (500 mg, 50%). MS: 716 m/z [M+H]⁺. ¹H NMR (400 MHz, CDCl₃) δ 7.81-7.75 (m, 4H), 7.61 (d, J=3.6 Hz, 1H), 7.33-7.28 (m, 4H), 7.11-6.88 (m, 5H), 6.58-6.56 (m, 1H), 6.20 (d, J=2.0 Hz, 1H), 4.57 (q, J=7.2 Hz, 1H), 4.15-4.09 (m, 2H), 2.97 (t, J=7.6 Hz, 2H), 2.62 (t, J=8.0 Hz, 2H), 2.39 (t, J=7.2 Hz, 6H), 1.65 (t, J=7.2 Hz, 3H), 1.22-1.21 (m, 3H) ppm.

### F. 3-(2-Fluoro-3-(1-(1-(3-((6-fluoro-4-(methylsulfonyl)-1H-indol-5-yl)oxy)phenyl)-1H-pyrazol-3-yl)ethyl)phenyl)propanoic acid

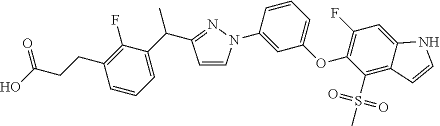 [SMILES: CC(c1ccn(-c2cccc(Oc3c(F)cc4[nH]ccc4c3S(C)(=O)=O)c2)n1)c1cccc(CCC(=O)O)c1F]

Exchanging methyl 3-(3-((2-(2-fluoro-5-((6-fluoro-4-(methylthio)-1H-indol-5-yl)oxy)phenyl)-1H-imidazol-5-yl)methyl)phenyl)propanoate for ethyl 3-(2-fluoro-3-(1-(1-(3-((6-fluoro-4-(methylthio)-1-tosyl-1H-indol-5-yl)oxy)phenyl)-1H-pyrazol-3-yl)ethyl)phenyl)propanoate, the two step reaction sequence described in Step B and C for Example 22 was used to prepare the title compound as a white solid. MS: 566 m/z [M+H]⁺.

### G. Example 23A, Enantiomer 1 and Example 23B, Enantiomer 2 of 3-(2-Fluoro-3-(1-(1-(3-((6-fluoro-4-(methylsulfonyl)-1H-indol-5-yl)oxy)phenyl)-1H-pyrazol-3-yl)ethyl)phenyl)propanoic acid

Racemic 3-(2-fluoro-3-(1-(1-(3-((6-fluoro-4-(methylsulfonyl)-1H-indol-5-yl)oxy)phenyl)-1H-pyrazol-3-yl)ethyl)phenyl)propanoic acid was separated by chiral SFC using chiral OJ (20×250 mm, 10 micron), eluting with 30% carbon dioxide and 70% methanol (0.2% ammonia saturated methanol), at a pressure of 100 bar, and flow rate of 80 g/min.

Peak 1: Example 23A, Enantiomer 1 of 3-(2-Fluoro-3-(1-(1-(3-((6-fluoro-4-(methylsulfonyl)-1H-indol-5-yl)oxy)phenyl)-1H-pyrazol-3-yl)ethyl)phenyl)propanoic acid. MS: 566 m/z [M+H]⁺. ¹H NMR (400 MHz, CD₃OD) δ 8.07 (d, J=2.4 Hz, 1H), 7.66 (d, J=5.2 Hz, 1H), 7.54 (d, J=3.2 Hz, 1H), 7.43-7.36 (m, 3H), 7.20 (d, J=3.6 Hz, 1H), 7.16-7.10 (m, 2H), 7.02-6.98 (m, 1H), 6.79 (d, J=7.6 Hz, 1H), 6.30 (d, J=2.4 Hz, 1H), 4.55 (q, J=7.2 Hz, 1H), 3.33-3.32 (m, 3H), 2.95 (t, J=7.6 Hz, 2H), 2.60 (t, J=7.6 Hz, 2H), 1.64 (d, J=7.2 Hz, 3H) ppm.

Peak 2: Example 23B, Enantiomer 2 of 3-(2-Fluoro-3-(1-(1-(3-((6-fluoro-4-(methylsulfonyl)-1H-indol-5-yl)oxy)phenyl)-1H-pyrazol-3-yl)ethyl)phenyl)propanoic acid. MS: 566 m/z [M+H]⁺.

¹H NMR (400 MHz, CD₃OD) δ 8.07 (d, J=2.4 Hz, 1H), 7.66 (d, J=5.2 Hz, 1H), 7.54 (d, J=3.2 Hz, 1H), 7.43-7.36 (m, 3H), 7.20 (d, J=3.6 Hz, 1H), 7.16-7.10 (m, 2H), 7.02-6.98 (m, 1H), 6.79 (d, J=7.6 Hz, 1H), 6.30 (d, J=2.4 Hz, 1H), 4.55 (q, J=7.2 Hz, 1H), 3.33-3.32 (m, 3H), 2.95 (t, J=7.6 Hz, 2H), 2.60 (t, J=7.6 Hz, 2H), 1.64 (d, J=7.2 Hz, 3H) ppm.

### Example 24

### 3-(3-((2-(2-Fluoro-5-((6-fluoro-4-(methylsulfonyl)-1H-indol-5-yl)oxy)phenyl)-1H-imidazol-5-yl)methyl)phenyl)propanamide

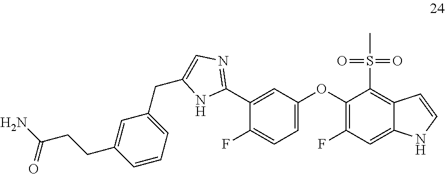 [SMILES: CS(=O)(=O)c1c(Oc2ccc(F)c(-c3ncc(Cc4cccc(CCC(N)=O)c4)[nH]3)c2)c(F)cc2[nH]ccc12]

To a 2 mL microwave vial containing 3-(3-((2-(2-fluoro-5-((6-fluoro-4-(methylsulfonyl)-1H-indol-5-yl)oxy)phenyl)-1H-imidazol-5-yl)methyl)phenyl)propanoic acid (Example 22, 20 mg, 0.036 mmol), ammonium chloride (58 mg, 1.08 mmol) and HATU (41 mg, 0.108 mmol) in DMF (2 mL) was added triethylamine (0.15 mL, 1.08 mmol). The vial was sealed, the contents stirred at room temperature for two hours and then diluted with ethyl acetate (30 mL). The organic layer was washed with brine (10 mL×3), dried over sodium sulfate, filtered and concentrated. The resulting residue was purified by flash chromatography over silica (0-100% ethyl acetate in petroleum ether) to afford the title compound as a solid (11.2 mg, 56%). MS: 551 m/z [M+H]⁺. ¹H NMR (400 MHz, CD₃OD) δ 7.67 (d, J=10.6 Hz, 1H), 7.55-7.49 (m, 2H), 7.25 (d, J=10.9 Hz, 1H), 7.23-7.19 (m, 2H), 7.15 (s, 1H), 7.10 (s, 2H), 7.03 (d, J=8.8 Hz, 1H), 6.84 (s, 1H), 3.91 (s, 2H), 3.31 (s, 3H), 2.87 (t, J=7.6 Hz, 2H), 2.46 (t, J=7.7 Hz, 2H) ppm.

### Example 25

### 3-(3-((5-(3-((4-((Difluoromethyl)sulfinyl)-6-fluoro-1H-indol-5-yl)oxy)phenyl)-4H-1,2,4-triazol-3-yl)methyl)phenyl)propanoic acid

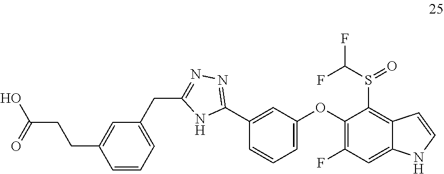 [SMILES: O=C(O)CCc1cccc(Cc2nnc(-c3cccc(Oc4c(F)cc5[nH]ccc5c4S(=O)C(F)F)c3)[nH]2)c1]

### A. 3-((4-((Difluoromethyl)thio)-6-fluoro-1H-indol-5-yl)oxy)benzonitrile

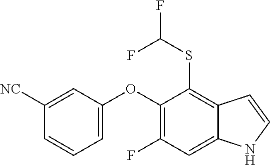 [SMILES: N#Cc1cccc(Oc2c(F)cc3[nH]ccc3c2SC(F)F)c1]

3-((6-Fluoro-4-mercapto-1-tosyl-1H-indol-5-yl)oxy)benzonitrile (500 mg, 1.14 mmol, prepared in two steps from 3-hydroxybenzonitrile and 3-((4-bromo-6-fluoro-1-tosyl-1H-indol-5-yl)oxy)benzonitrile (Intermediate 7-2) as described for Intermediate 23B, Steps A to B), ClCF₂COONa (348 mg, 2.28 mmol) and potassium carbonate (315 mg, 2.28 mmol) in DMF (25 mL) and water (2.5 mL) was heated at 130° C. for one hour. The reaction mixture was cooled to room temperature, diluted with ethyl acetate (30 mL), washed with water (10 mL×3) and brine (10 mL), dried over sodium sulfate, filtered and concentrated. The resulting residue was purified by flash chromatography over silica (petroleum ether/ethyl acetate, v/v, 3/1) to afford the title compound as a solid (200 mg, 50%). MS: 335 m/z [M+H]⁺.

### B. 4-((Difluoromethyl)thio)-6-fluoro-5-(3-(5-(3-iodobenzyl)-4H-1,2,4-triazol-3-yl)phenoxy)-1H-indole

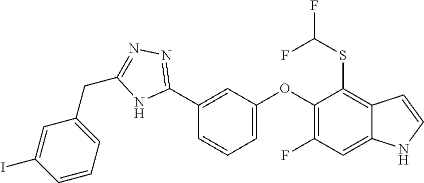 [SMILES: Fc1cc2[nH]ccc2c(SC(F)F)c1Oc1cccc(-c2nnc(Cc3cccc(I)c3)[nH]2)c1]

To a 5 mL microwave vial was added 3-((4-((difluoromethyl)thio)-6-fluoro-1H-indol-5-yl)oxy)benzonitrile (250 mg, 0.75 mmol), 2-(3-iodophenyl)acetohydrazide (310 mg, 1.12 mmol, Intermediate 25), potassium carbonate (207 mg, 1.5 mmol) and 3 mL of n-BuOH. The reaction was heated at 160° C. in a microwave reactor for five hours. The reaction mixture was diluted with diethyl ether (25 mL), washed with brine, dried over sodium sulfate and concentrated. The resulting residue was purified by flash chromatography over silica (petroleum ether/ethyl acetate, v/v, 1/1) to afford the title compound as brown solid (150 mg, 34%). MS m/z: 593 m/z [M+H]⁺.

### C. Ethyl 3-(3-((5-(3-((4-((difluoromethyl)thio)-6-fluoro-1H-indol-5-yl)oxy)phenyl)-4H-1,2,4-triazol-3-yl)methyl)phenyl)propanoate

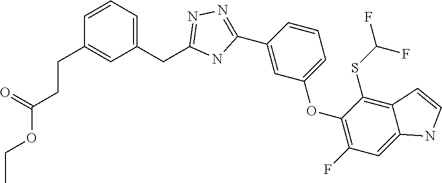 [SMILES: CCOC(=O)CCc1cccc(Cc2nnc(-c3cccc(Oc4c(F)cc5[nH]ccc5c4SC(F)F)c3)[nH]2)c1]

To a stirred solution of 4-((difluoromethyl)thio)-6-fluoro-5-(3-(5-(3-iodobenzyl)-4H-1,2,4-triazol-3-yl)phenoxy)-1H-indole (200 mg, 0.34 mmol), 3,3-diethoxyprop-1-ene (132 mg, 1 mmol) and tributylamine (125 mg, 0.68 mmol) in DMF (2.5 mL) was added tetrabutylammonium chloride (94 mg, 0.34 mmol) followed by palladium(II) acetate (8 mg, 0.037 mmol). The reaction mixture was heated at 90° C. overnight, cooled to room temperature, acidified with 2 N hydrochloric acid to pH 5 and extracted with ethyl acetate (3×10 mL). The combined organic extracts were washed with water (35 mL), brine, dried over sodium sulfate, and concentrated. The resulting residue was purified by flash chromatography over silica (petroleum ether/ethyl acetate, v/v, 1/10) to give the title compound as a yellow solid (100 mg, 52%). MS: 567 m/z [M+H]⁺.

### D. Ethyl 3-(3-((5-(3-((4-((difluoromethyl)sulfinyl)-6-fluoro-1H-indol-5-yl)oxy)phenyl)-4H-1,2,4-triazol-3-yl)methyl)phenyl)propanoate

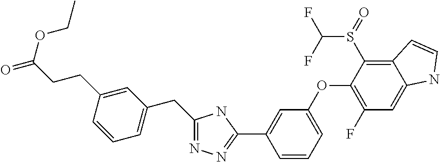 [SMILES: CCOC(=O)CCc1cccc(Cc2nnc(-c3cccc(Oc4c(F)cc5[nH]ccc5c4S(=O)C(F)F)c3)[nH]2)c1]

To a solution of ethyl 3-(3-((5-(3-((4-((difluoromethyl)thio)-6-fluoro-1H-indol-5-yl)oxy)phenyl)-4H-1,2,4-triazol-3-yl)methyl)phenyl)propanoate (130 mg, 0.23 mmol) in methanol (10 mL) was added a solution of ammonium molybdate tetrahydrate (400 mg) in 2 mL of hydrogen peroxide (30% in water) and stirred at room temperature for fix hours. The reaction mixture was diluted with ethyl acetate (50 mL), washed with water (20 mL×3), aq. sodium sulfite (20 mL) and brine (10 mL×2), dried over sodium sulfate and concentrated. The resulting residue was purified by preparative HPLC to give the title compound as a yellow solid (25 mg, 13%). MS: 583 m/z [M+H]⁺.

### E. Example 25, 3-(3-((5-(3-((4-((Difluoromethyl)sulfinyl)-6-fluoro-1H-indol-5-yl)oxy)phenyl)-4H-1,2,4-triazol-3-yl)methyl)phenyl)propanoic acid

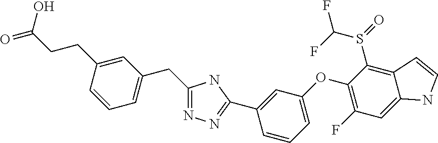 [SMILES: O=C(O)CCc1cccc(Cc2nnc(-c3cccc(Oc4c(F)cc5[nH]ccc5c4S(=O)C(F)F)c3)[nH]2)c1]

To a solution of ethyl 3-(3-((5-(3-((4-((difluoromethyl)sulfinyl)-6-fluoro-1H-indol-5-yl)oxy)phenyl)-4H-1,2,4-triazol-3-yl)methyl)phenyl)propanoate (25 mg, 0.043 mmol) in methanol (1 mL) and THF (3 mL) was added lithium hydroxide (1M in water, 1 mL). The mixture was stirred at room temperature for two hours, acidified with 1N hydrochloric acid to pH˜4, diluted with ethyl acetate (50 mL), washed with brine (10 mL×3), dried over sodium sulfate, filtered and concentrated. The resulting residue was purified by preparative HPLC to give the title compound as a solid (4.5 mg, 18%). LC-MS Retention time: 1.07 minutes. MS: 555 m/z [M+H]⁺. ¹H NMR (400 MHz, CD₃OD) δ 7.79-7.72 (m, 1H), 7.66 (d, J=10.8 Hz, 1H), 7.56-7.42 (m, 3H), 7.29-7.04 (m, 6H), 6.99 (t, J=54.3 Hz, 1H), 4.13 (s, 2H), 2.90 (t, J=7.9 Hz, 2H), 2.57 (t, J=7.9 Hz, 2H) ppm.

### Example 26

### 3-(3-(1-(2-(2-Fluoro-5-((6-fluoro-4-(methylsulfonyl)-1H-indol-5-yl)oxy)phenyl)-1H-imidazol-5-yl)-1-hydroxyethyl)phenyl)propanoic acid

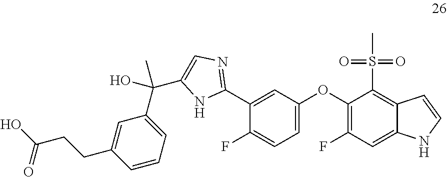 [SMILES: CC(O)(c1cccc(CCC(=O)O)c1)c1cnc(-c2cc(Oc3c(F)cc4[nH]ccc4c3S(C)(=O)=O)ccc2F)[nH]1]

### A. Methyl 3-(3-(1-(2-(2-fluoro-5-((6-fluoro-4-(methylthio)-1H-indol-5-yl)oxy)phenyl)-1H-imidazol-5-yl)-1-hydroxyethyl)phenyl)propanoate

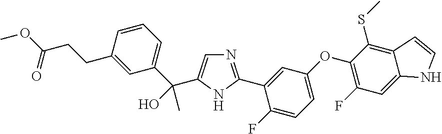 [SMILES: COC(=O)CCc1cccc(C(C)(O)c2cnc(-c3cc(Oc4c(F)cc5[nH]ccc5c4SC)ccc3F)[nH]2)c1]

A solution of 2-fluoro-5-((6-fluoro-4-(methylthio)-1H-indol-5-yl)oxy)benzimidamide (Intermediate 24-4, 300 mg) and methyl 3-(3-(3-formyl-2-methyloxiran-2-yl)phenyl)propanoate (Intermediate 26, 200 mg, 0.8 mmol) in DMF (6 mL) was stirred at 75° C. overnight. The reaction mixture was diluted with water (50 mL) and extracted with ethyl acetate (30 mL×3). The extracts were washed with brine (20 mL), dried over sodium sulfate, filtered and concentrated. The resulting residue was purified by flash silica gel column chromatography (0-100% ethyl acetate in petroleum ether) to give the title compound as a yellow solid (150 mg, 35%). MS: 564 m/z [M+H]⁺.

### B. Methyl 3-(3-(1-(2-(2-fluoro-5-((6-fluoro-4-(methylsulfonyl)-1H-indol-5-yl)oxy)phenyl)-1H-imidazol-5-yl)-1-hydroxyethyl)phenyl)propanoate

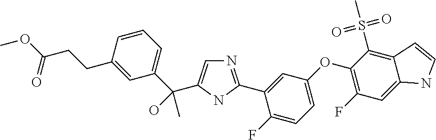 [SMILES: COC(=O)CCc1cccc(C(C)(O)c2cnc(-c3cc(Oc4c(F)cc5[nH]ccc5c4S(C)(=O)=O)ccc3F)[nH]2)c1]

To a solution of methyl 3-(3-(1-(2-(2-fluoro-5-((6-fluoro-4-(methylthio)-1H-indol-5-yl)oxy)phenyl)-1H-imidazol-5-yl)-1-hydroxyethyl)phenyl)propanoate (150 mg, 0.266 mmol) in methanol (7 mL) was added a solution of ammonium molybdate tetrahydrate (200 mg) in 1 mL of hydrogen peroxide (30% in water) and stirred at room temperature for one hour. The reaction mixture was diluted with ethyl acetate (70 mL), washed with water (20 mL×3), aqueous sodium sulfite (20 mL), brine (10 mL×2), dried over sodium sulfate and concentrated. The resulting residue was purified by flash silica gel column chromatography (0-100% ethyl acetate in petroleum ether) to give the title compound as a yellow solid (35 mg, 22%). MS: 596 m/z [M+H]⁺.

### C. Example 26. 3-(3-(1-(2-(2-Fluoro-5-((6-fluoro-4-(methylsulfonyl)-1H-indol-5-yl)oxy)phenyl)-1H-imidazol-5-yl)-1-hydroxyethyl)phenyl)propanoic acid

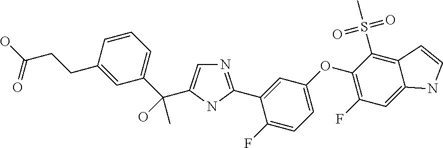 [SMILES: CC(O)(c1cccc(CCC(=O)O)c1)c1cnc(-c2cc(Oc3c(F)cc4[nH]ccc4c3S(C)(=O)=O)ccc2F)[nH]1]

To a solution of methyl 3-(3-(1-(2-(2-fluoro-5-((6-fluoro-4-(methylsulfonyl)-1H-indol-5-yl)oxy)phenyl)-1H-imidazol-5-yl)-1-hydroxyethyl)phenyl)propanoate (35 mg, 0.059 mmol) in methanol (1 mL) and THF (3 mL) was added lithium hydroxide (1M in water, 1 mL). The reaction mixture was stirred at room temperature for two hours, neutralized with 1 N hydrochloric acid to pH˜2 and then concentrated. The resulting residue was diluted with water (15 mL), basified with 2N sodium hydroxide to pH˜11 and washed with ether (10 mL). The aqueous layer was acidified with 1N hydrochloric acid to pH˜4, diluted with ethyl acetate (50 mL) and washed with brine (10 mL×3). The organic layer was dried over sodium sulfate, filtered and concentrated to afford the title compound as a solid (20 mg, 59%). MS: 582 m/z [M+H]⁺. ¹H NMR (400 MHz, CD₃OD) δ 7.66 (d, J=10.5 Hz, 1H), 7.53 (d, J=3.2 Hz, 2H), 7.38 (s, 1H), 7.24 (dt, J=22.0, 9.3 Hz, 41H), 7.12 (d, J=6.9 Hz, 1H), 6.97 (d, J=11.1 Hz, 21H), 3.32 (s, 31H), 2.94-2.87 (m, 21H), 2.58 (t, J=7.7 Hz, 21H), 1.88 (s, 31H) ppm.

The following examples were prepared utilizing the procedures described for Example 26.

| | Ex. | | | LC-MS Ret | MS m/z |
|---|---|---|---|---|---|
| Structure | No. | Name | ¹H NMR | time (min) | [M + H]⁺ |
| 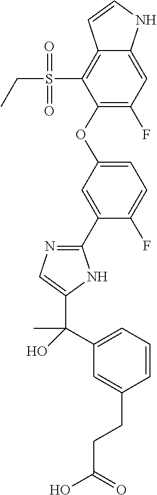 [SMILES: CCS(=O)(=O)c1c(Oc2ccc(F)c(-c3ncc(C(C)(O)c4cccc(CCC(=O)O)c4)[nH]3)c2)c(F)cc2[nH]ccc12] | 27 | 3-(3-(1-(2-(5-((4- (ethylsulfonyl)-6- fluoro-1H-indol-5- yl)oxy)-2- fluorophenyl)-1H- imidazol-5-yl)-1- hydroxyethyl)phenyl) propanoic acid | (400 MHz, CD₃OD) δ 7.67 (d, J = 10.2 Hz, 1H), 7.54 (d, J = 2.9 Hz, 2H), 7.39 (s, 1H), 7.32-7.16 (m, 4H), 7.13 (d, J = 7.5 Hz, 1H), 7.00- 6.92 (m, 2H), 3.50-3.44 (m, 2H), 2.94 (t, J = 7.8 Hz, 2H), 2.59 (t, J = 7.8 Hz, 2H), 1.89 (s, 3H), 1.27 (t, J = 7.2 Hz, 3H) ppm. | 0.81 | 596 |
| 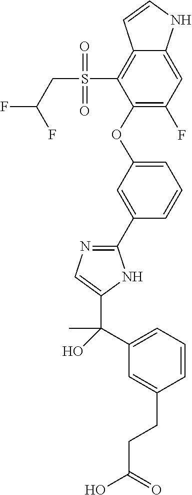 [SMILES: CC(O)(c1cccc(CCC(=O)O)c1)c1cnc(-c2cccc(Oc3c(F)cc4[nH]ccc4c3S(=O)(=O)CC(F)F)c2)[nH]1] | 28 | 3-(3-(1-(2-(3-((4-((2,2- difluoroethyl)sulfonyl)- 6-fluoro-1H-indol-5- yl)oxy)phenyl)-1H- imidazol-5-yl)-1- hydroxyethyl)phenyl) propanoic acid | (400 MHz, CD₃OD) δ 7.71 (d, J = 10.4 Hz, 1H), 7.64-7.49 (m, 3H), 7.41 (d, J = 12.2 Hz, 2H), 7.32- 7.17 (m, 3H), 7.13 (d, J = 6.8 Hz, 1H), 6.98 (d, J = 6.4 Hz, 1H), 6.92 (s, 1H), 6.29 (m, 1H), 4.16 (m, 2H), 2.92 (t, J = 7.6 Hz, 2H), 2.59 (t, J = 7.6 Hz, 2H), 1.90 (s, 3H) ppm. | 0.83 | 614 |
| 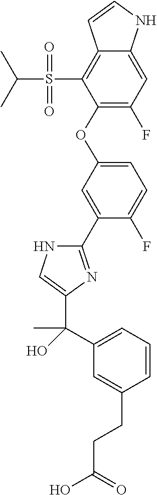 [SMILES: CC(C)S(=O)(=O)c1c(Oc2ccc(F)c(-c3nc(C(C)(O)c4cccc(CCC(=O)O)c4)c[nH]3)c2)c(F)cc2[nH]ccc12] | 29 | 3-(3-(1-(2-(2-fluoro-5- ((6-fluoro-4- (isopropylsulfonyl)- 1H-indol-5- yl)oxy)phenyl)-1H- imidazol-4-yl)-1- hydroxyethyl)phenyl) propanoic acid | (400 MHz, CD₃OD) δ 7.67 (d, J = 10.6 Hz, 1H), 7.54-7.51 (m, 2H), 7.39-7.37 (m, 1H), 7.31-7.10 (m, 5H), 6.96 (s, 1H), 6.94-6.91 (m, 1H), 3.75-3.70 (m, 1H), 2.90 (t, J = 7.6 Hz, 2H), 2.58 (t, J = 7.6 Hz, 2H), 1.88 (s, 3H), 1.30 (d, J = 6.8 Hz, 6H) ppm. | 0.90 | 610 |

### Example 30

### 3-(3-(1-(2-(2-Fluoro-5-((6-fluoro-4-(methylsulfonyl)-1H-indol-5-yl)oxy)phenyl)-1H-imidazol-5-yl)-1-hydroxyethyl)phenyl)propanamide

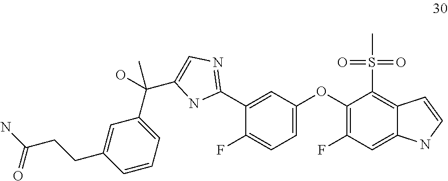 [SMILES: CC(O)(c1cccc(CCC(N)=O)c1)c1cnc(-c2cc(Oc3c(F)cc4[nH]ccc4c3S(C)(=O)=O)ccc2F)[nH]1]

Triethylamine (0.11 mL, 0.78 mmol) was added to a 2 mL microwave vial containing a solution of 3-(3-(1-(2-(2-fluoro-5-((6-fluoro-4-(methylsulfonyl)-1H-indol-5-yl)oxy)phenyl)-1H-imidazol-5-yl)-1-hydroxyethyl)phenyl)propanoic acid (Example 26, 15 mg, 0.026 mmol), ammonium chloride (42 mg, 0.78 mmol) and HATU (30 mg, 0.078 mmol) in DMF (2 mL). The vial was sealed, the reaction mixture was stirred at room temperature for two hours and then diluted with ethyl acetate (30 mL). The organic layer was washed with brine (10 mL×3), dried over sodium sulfate, filtered and concentrated. The resulting residue was purified by flash silica gel column chromatography (0-10% methanol in dichloromethane). The product was further purified by preparative HPLC to afford the title compound as a solid (3.4 mg, 23%). MS: 581 m/z [M+H]⁺. ¹H NMR (400 MHz, CD₃OD) δ 7.53 (d, J=10.4 Hz, 1H), 7.45-7.40 (m, 2H), 7.24 (s, 1H), 7.18 (d, J=7.8 Hz, 1H), 7.13-7.06 (m, 3H), 7.00 (d, J=7.3 Hz, 1H), 6.87-6.84 (m, 2H), 3.33 (s, 3H), 2.78 (t, J=8 Hz, 2H), 2.40-2.36 (t, J=8 Hz, 2H), 1.75 (s, 3H) ppm.

### Example 31

### 3-(3-((3-(3-((6-Fluoro-4-(methylsulfonyl)-1H-indol-5-yl)oxy)phenyl)-1-methyl-1H-1,2,4-triazol-5-yl)methyl)phenyl)propanoic acid

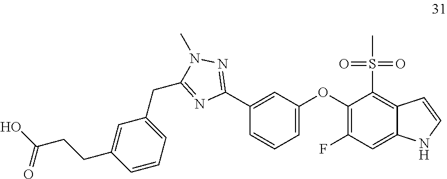 [SMILES: Cn1nc(-c2cccc(Oc3c(F)cc4[nH]ccc4c3S(C)(=O)=O)c2)nc1Cc1cccc(CCC(=O)O)c1]

### A. Tert-butyl 2-(2-(3-iodophenyl)acetyl)-2-methylhydrazine-1-carboxylate

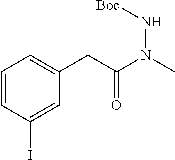 [SMILES: CN(NC(=O)OC(C)(C)C)C(=O)Cc1cccc(I)c1]

To a stirred solution of 2-(3-iodophenyl)acetic acid (5 g, 0.019 mol) in dichloromethane (50 mL) were added oxalyl chloride (3.6 g, 0.028 mol) and three drops of DMF. The resulting mixture was stirred at room temperature for 1.5 hours and then concentrated. The residue was dissolved in dichloromethane (10 mL) and added dropwise to a solution of tert-butyl 2-methylhydrazine-1-carboxylate (3.3 g, 0.023 mol) and diisoproylethylamine (12 g, 0.095 mol) in dichloromethane (30 mL). The reaction mixture was stirred at room temperature overnight and then concentrated. The resulting residue was purified by flash silica gel column chromatography (petroleum ether/ethyl acetate, v/v, 2/1) to give the title compound as a yellow solid (5 g, 67%). MS: 391 m/z [M+H]⁺.

### B. 2-(3-Iodophenyl)-N-methylacetohydrazide hydrochloride

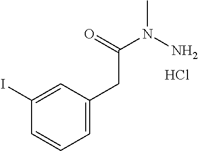

Tert-butyl 2-(2-(3-iodophenyl)acetyl)-2-methylhydrazine-1-carboxylate (5 g, 0.0128 mol) was added to a 4 M solution of hydrochloric acid in 1.4-dioxane (30 mL) and the mixture was stirred at room temperature for four hours. The precipitate was collected by filtration, rinsed with ether and dried to give the title compound (3.9 g, 92%). MS: 291 m/z [M+H]⁺.

### C. 6-Fluoro-5-(3-(5-(3-iodobenzyl)-1-methyl-1H-1,2,4-triazol-3-yl)phenoxy)-4-(methylthio)-1-tosyl-1H-indole

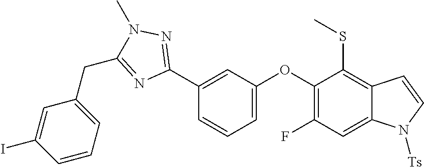 [SMILES: CSc1c(Oc2cccc(-c3nc(Cc4cccc(I)c4)n(C)n3)c2)c(F)cc2c1ccn2S(=O)(=O)c1ccc(C)cc1]

To a 5 mL microwave vial containing a solution of 3-((6-fluoro-4-(methylthio)-1-tosyl-1H-indol-5-yl)oxy)benzonitrile (Intermediate 23C-1, 150 mg, 0.33 mmol) and 2-(3-iodophenyl)-N-methylacetohydrazide (290 mg, 1 mmol) in THF (2.5 mL) was added bistrimethylaluminum-1,4-diazabicyclo[2.2.2]octane complex (167 mg, 0.66 mmol) and heated at 130° C. for six hours in a microwave reactor. The reaction mixture was poured into water (30 mL) and extracted with ethyl acetate (20 mL×3). The combined organic extracts were washed with brine (20 mL), dried over sodium sulfate, filtered and concentrated. The resulting residue was purified by flash silica gel column chromatography (0-100% ethyl acetate in petroleum ether) to give the title compound as a yellow solid (48 mg, 20%). MS: 725 m/z [M+H]⁺

### D. Ethyl 3-(3-((3-(3-((6-fluoro-4-(methylthio)-1-tosyl-1H-indol-5-yl)oxy)phenyl)-1-methyl-1H-1,2,4-triazol-5-yl)methyl)phenyl)propanoate

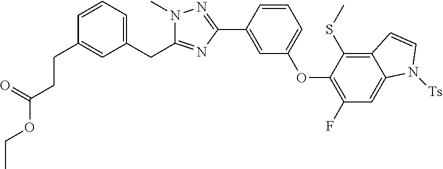 [SMILES: CCOC(=O)CCc1cccc(Cc2nc(-c3cccc(Oc4c(F)cc5c(ccn5S(=O)(=O)c5ccc(C)cc5)c4SC)c3)nn2C)c1]

To a stirred solution of 6-fluoro-5-(3-(5-(3-iodobenzyl)-1-methyl-1H-1,2,4-triazol-3-yl)phenoxy)-4-(methylthio)-1-tosyl-1H-indole (48 mg, 0.066 mmol), 3,3-diethoxyprop-1-ene (25 mg, 0.199 mmol) and tributylamine (25 mg, 0.132 mmol) in DMF (1.5 mL) were added tetrabutylammonium chloride (18 mg, 0.066 mmol) and palladium(II) acetate (4.5 mg, 0.02 mmol). The reaction mixture was heated at 90° C. overnight, cooled to room temperature and diluted with ethyl acetate (50 mL). The organic layer was washed with brine (15 mL×3), dried over sodium sulfate, filtered and concentrated. The residue was purified by flash chromatography over silica (0-100% ethyl acetate in petroleum ether) to give the title compound as a yellow solid (27 mg, 58%). MS: 699 m/z [M+H]⁺.

### E. Example 31, 3-(3-((3-(3-((6-Fluoro-4-(methylsulfonyl)-1H-indol-5-yl)oxy)phenyl)-1-methyl-1H-1,2,4-triazol-5-yl)methyl)phenyl)propanoic acid

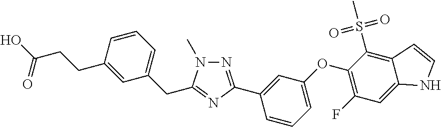 [SMILES: Cn1nc(-c2cccc(Oc3c(F)cc4[nH]ccc4c3S(C)(=O)=O)c2)nc1Cc1cccc(CCC(=O)O)c1]

To a solution of ethyl 3-(3-((3-(3-((6-fluoro-4-(methylthio)-1-tosyl-1H-indol-5-yl)oxy)phenyl)-1-methyl-1H-1,2,4-triazol-5-yl)methyl)phenyl)propanoate (27 mg, 0.0387 mmol) in methanol (2 mL) was added a solution of ammonium molybdate tetrahydrate (100 mg) in 1 mL of hydrogen peroxide (30% in water) and stirred at room temperature for three hours. Reaction mixture was diluted with ethyl acetate (60 mL), washed with water (20 mL×3), aq. sodium sulfite (20 mL) and brine (10 mL×2), dried over sodium sulfate and concentrated to give crude ethyl 3-(3-((3-(3-((6-fluoro-4-(methylsulfonyl)-1-tosyl-1H-indol-5-yl)oxy)phenyl)-1-methyl-1H-1,2,4-triazol-5-yl)methyl)phenyl)propanoate as a yellow solid (20 mg, 19%), which was used in next step without further purification. MS: 731 m/z [M+H]⁺. To a solution of the above ethyl 3-(3-((3-(3-((6-fluoro-4-(methylsulfonyl)-1-tosyl-1H-indol-5-yl)oxy)phenyl)-1-methyl-1H-1,2,4-triazol-5-yl)methyl)phenyl)propanoate (20 mg) in THF/water (1 mL/1 mL) was added lithium hydroxide (10 mg) and the reaction mixture was stirred in a microwave reactor at 120° C. for one hour. The reaction mixture was acidified with 1N hydrochloric acid to pH˜4, diluted with ethyl acetate (50 mL), washed with brine (10 mL×3), dried over sodium sulfate, filtered and concentrated. The resulting residue was purified by flash chromatography over silica (0-10% methanol in dichloromethane) to give the title compound, 3-(3-((3-(3-((6-fluoro-4-(methylsulfonyl)-1H-indol-5-yl)oxy)phenyl)-1-methyl-1H-1,2,4-triazol-5-yl)methyl)phenyl)propanoic acid as a solid (7.2 mg, 36% based on 48 mg of 6-fluoro-5-(3-(5-(3-iodobenzyl)-1-methyl-1H-1,2,4-triazol-3-yl)phenoxy)-4-(methylthio)-1-tosyl-1H-indole). MS: 549 m/z [M+H]⁺. ¹H NMR (400 MHz, CD₃OD) δ 7.75 (d, J=7.9 Hz, 1H), 7.67 (d, J=10.5 Hz, 1H), 7.56 (s, 1H), 7.53 (d, J=3.2 Hz, 1H), 7.42 (t, J=8.0 Hz, 1H), 7.26 (t, J=7.6 Hz, 1H), 7.21 (d, J=3.2 Hz, 1H), 7.16 (s, 1H), 7.14 (s, 1H), 7.05 (t, J=6.7 Hz, 2H), 4.22 (s, 2H), 3.75 (s, 3H), 3.34 (s, 3H), 2.90 (t, J=7.6 Hz, 2H), 2.58 (t, J=7.5 Hz, 2H) ppm.

### Examples 32 and 33

### 3-(3-((3-(3-((6-Fluoro-4-(methylsulfonyl)-1H-indol-5-yl)oxy)phenyl)-1H-1,2,4-triazol-1-yl)methyl)phenyl)propanoic acid

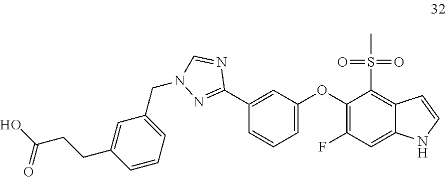 [SMILES: CS(=O)(=O)c1c(Oc2cccc(-c3ncn(Cc4cccc(CCC(=O)O)c4)n3)c2)c(F)cc2[nH]ccc12]

### 3-(3-((3-(3-((6-Fluoro-4-(methylsulfinyl)-1H-indol-5-yl)oxy)phenyl)-1H-1,2,4-triazol-1-yl)methyl)phenyl)propanoic acid

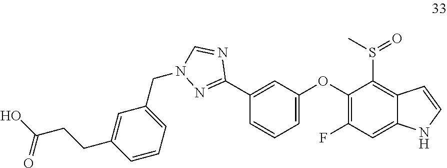 [SMILES: CS(=O)c1c(Oc2cccc(-c3ncn(Cc4cccc(CCC(=O)O)c4)n3)c2)c(F)cc2[nH]ccc12]

### A. Methyl 3-((6-fluoro-4-(methylthio)-1-tosyl-1H-indol-5-yl)oxy)benzimidothioate

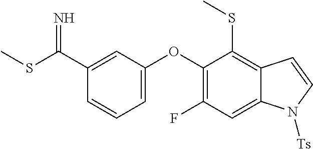 [SMILES: CSC(=N)c1cccc(Oc2c(F)cc3c(ccn3S(=O)(=O)c3ccc(C)cc3)c2SC)c1]

To a solution of 3-((6-fluoro-4-(methylthio)-1-tosyl-1H-indol-5-yl)oxy)benzothioamide (Intermediate 33, 1.1 g, 1.58 mmol) in diethyl ether (10 mL) was added iodomethane (0.4 mL), stirred at room temperature for 18 hours and then filtered. The filtrate was concentrated to afford crude methyl 3-((6-fluoro-4-(methylthio)-1-tosyl-1H-indol-5-yl)oxy)benzimidothioate as a yellow solid (650 mg). MS: 501 m/z [M+H]⁺.

### B. 5-(3-(4H-1,2,4-Triazol-3-yl)phenoxy)-6-fluoro-4-(methylthio)-1-tosyl-1H-indole

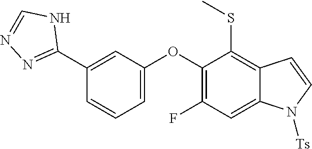 [SMILES: CSc1c(Oc2cccc(-c3nnc[nH]3)c2)c(F)cc2c1ccn2S(=O)(=O)c1ccc(C)cc1]

To a solution of methyl 3-((6-fluoro-4-(methylthio)-1-tosyl-1H-indol-5-yl)oxy)benzimidothioate (650 mg, 80% purity, 1.04 mmol) in DMF (4 mL) was added formic hydrazide (125 mg, 2.08 mmol) and heated at 90° C. for two hours. The reaction mixture was quenched with ice water (30 mL) and extracted with ethyl acetate (50 mL×3). The combined organic extracts were washed with brine (15 mL×3), dried over sodium sulfate, filtered and concentrated. The resulting residue was purified by flash chromatography over silica (0-60% of ethyl acetate in petroleum ether) to give the title compound (500 mg, 73%). MS: 495 m/z [M+H]⁺. ¹H NMR (400 MHz, CDCl₃) δ 8.18 (s, 1H), 7.84-7.77 (m, 3H), 7.73 (d, J=7.8 Hz, 1H), 7.62 (d, J=3.7 Hz, 1H), 7.56 (s, 1H), 7.38 (t, J=8.0 Hz, 1H), 7.30 (d, J=8.1 Hz, 2H), 6.92 (d, J=8.4 Hz, 1H), 6.88 (d, J=3.2 Hz, 1H), 2.41 (s, 3H), 2.40 (s, 3H) ppm.

### C. Ethyl 3-(3-(E3-(3-((6-fluoro-4-(methylthio)-1-tosyl-1H-indol-5-yl)oxy)phenyl)-1H-1,2,4-triazol-1-yl)methyl)phenyl)propanoate

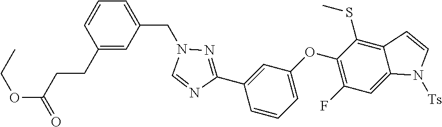 [SMILES: CCOC(=O)CCc1cccc(Cn2cnc(-c3cccc(Oc4c(F)cc5c(ccn5S(=O)(=O)c5ccc(C)cc5)c4SC)c3)n2)c1]

A solution of 5-(3-(4H-1,2,4-triazol-3-yl)phenoxy)-6-fluoro-4-(methylthio)-1-tosyl-1H-indole (400 mg, 0.61 mmol), ethyl 3-(3-(bromomethyl)phenyl)propanoate (164 mg, 0.61 mmol) and potassium carbonate (168 mg, 1.22 mmol) in DMF (5 mL) was stirred at room temperature for 18 hours. The reaction mixture was quenched with ice-water (30 mL) and extracted with ethyl acetate (50 mL×2). The combined organic extracts were washed with brine (50 mL×2), dried over sodium sulfate, filtered and concentrated. The resulting residue was purified by flash silica gel column chromatography (35-60% ethyl acetate in petroleum ether) to give the title compound as a yellow solid (360 mg, 74%). MS: 685 m/z [M+H]⁺. ¹H NMR (400 MHz, CDCl₃) δ 7.99 (s, 1H), 7.83-7.75 (m, 4H), 7.67 (s, 1H), 7.61 (d, J=3.7 Hz, 1H), 7.34-7.27 (m, 4H), 7.19 (d, J=7.7 Hz, 1H), 7.12 (d, J=7.0 Hz, 2H), 6.88 (d, J=3.6 Hz, 1H), 6.82 (dd, J=8.2, 2.5 Hz, 1H), 5.32 (s, 2H), 4.09 (q, J=7.1 Hz, 2H), 2.94 (t, J=7.7 Hz, 2H), 2.60 (t, J=7.7 Hz, 2H), 2.40 (s, 3H), 2.38 (s, 3H), 1.20 (t, J=7.1 Hz, 3H) ppm.

### D. Ethyl 3-(3-((3-(3-((6-fluoro-4-(methylsulfonyl)-1-tosyl-1H-indol-5-yl)oxy)phenyl)-1H-1,2,4-triazol-1-yl)methyl)phenyl)propanoate and ethyl 3-(3-((3-(3-((6-fluoro-4-(methylsulfinyl)-1-tosyl-1H-indol-5-yl)oxy)phenyl)-1H-1,2,4-triazol-1-yl)methyl)phenyl)propanoate

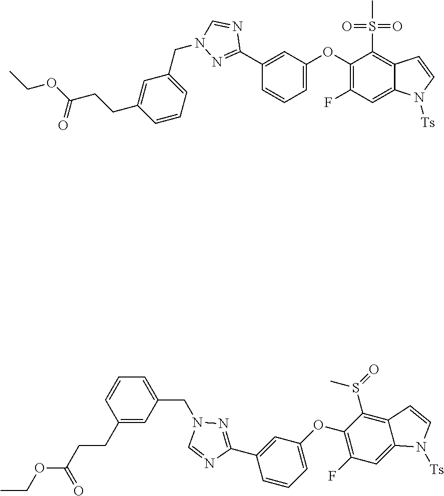 [SMILES: CCOC(=O)CCc1cccc(Cn2cnc(-c3cccc(Oc4c(F)cc5c(ccn5S(=O)(=O)c5ccc(C)cc5)c4S(C)(=O)=O)c3)n2)c1 ; CCOC(=O)CCc1cccc(Cn2cnc(-c3cccc(Oc4c(F)cc5c(ccn5S(=O)(=O)c5ccc(C)cc5)c4S(C)=O)c3)n2)c1]

To a solution of ethyl 3-(3-((3-(3-((6-fluoro-4-(methylthio)-1-tosyl-1H-indol-5-yl)oxy)phenyl)-1H-1,2,4-triazol-1-yl)methyl)phenyl)propanoate (360 mg, 86% purity, 0.45 mmol) in methanol (10 mL) was added a solution of ammonium molybdate tetrahydrate (800 mg) in 4.5 mL of hydrogen peroxide (30% in water) and stirred at room temperature for 2.5 hours. The reaction mixture was diluted with ethyl acetate (100 mL) and the organic layer was washed with water (40 mL×2), aq. sodium sulfite (40 mL) and brine (40 mL×2), dried and concentrated. The resulting residue was purified by flash chromatography (0-100% ethyl acetate in petroleum ether) to give ethyl 3-(3-((3-(3-((6-fluoro-4-(methylsulfonyl)-1-tosyl-1H-indol-5-yl)oxy)phenyl)-1H-1,2,4-triazol-1-yl)methyl)phenyl)propanoate as a light yellow semisolids (240 mg, 74%) and ethyl 3-(3-((3-(3-((6-fluoro-4-(methylsulfinyl)-1-tosyl-1H-indol-5-yl)oxy)phenyl)-1H-1,2,4-triazol-1-yl)methyl)phenyl)propanoate as a light yellow semisolids (70 mg, 22%).

Ethyl 3-(3-((3-(3-((6-fluoro-4-(methylsulfonyl)-1-tosyl-1H-indol-5-yl)oxy)phenyl)-1H-1,2,4-triazol-1-yl)methyl)phenyl)propanoate: MS: 717 m/z [M+H]⁺. ¹H NMR (400 MHz, CDCl₃) δ 8.11 (d, J=10.4 Hz, 1H), 8.01 (s, 1H), 7.86 (d, J=7.8 Hz, 1H), 7.80 (s, 1H), 7.77 (s, 1H), 7.75 (s, 1H), 7.73 (d, J=3.8 Hz, 1H), 7.53 (d, J=3.8 Hz, 1H), 7.35 (t, J=8.0 Hz, 1H), 7.32-7.27 (m, 3H), 7.20 (d, J=7.6 Hz, 1H), 7.12 (d, J=7.9 Hz, 2H), 6.89 (dd, J=8.2, 2.4 Hz, 1H), 5.33 (s, 2H), 4.10 (q, J=7.2 Hz, 2H), 3.29 (s, 3H), 2.94 (t, J=7.7 Hz, 2H), 2.60 (t, J=7.7 Hz, 2H), 2.39 (s, 3H), 1.20 (t, J=7.1 Hz, 3H) ppm.

Ethyl 3-(3-((3-(3-((6-fluoro-4-(methylsulfinyl)-1-tosyl-1H-indol-5-yl)oxy)phenyl)-1H-1,2,4-triazol-1-yl)methyl)phenyl)propanoate: MS: 701 m/z [M+H]⁺.

### F. Example 32: 3-(3-((3-(3-((6-Fluoro-4-(methylsulfonyl)-1H-indol-5-yl)oxy)phenyl)-1H-1,2,4-triazol-1-yl)methyl)phenyl)propanoic acid

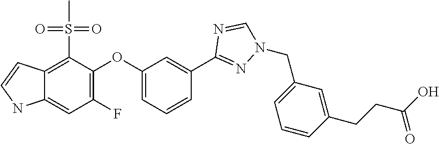 [SMILES: CS(=O)(=O)c1c(Oc2cccc(-c3ncn(Cc4cccc(CCC(=O)O)c4)n3)c2)c(F)cc2[nH]ccc12]

To a solution of ethyl 3-(3-((3-(3-((6-fluoro-4-(methylsulfonyl)-1-tosyl-1H-indol-5-yl)oxy)phenyl)-1H-1,2,4-triazol-1-yl)methyl)phenyl)propanoate (240 mg, 0.33 mmol) in water (3 mL) and THF (3 mL) was added lithium hydroxide (93 mg) and heated at 120° C. for 30 minutes in a microwave reactor. The reaction mixture was cooled to 0° C., acidified with 1 N hydrochloric acid to pH˜4 and extracted with ethyl acetate (50 mL×3). The combined organic extracts were washed with brine (30 mL×2), dried over sodium sulfate, filtered and concentrated. The resulting residue was purified by preparative HPLC to give the title compound as a white solid (52 mg, 30%). MS: 535 m/z [M+H]⁺. ¹H NMR (400 MHz, DMSO-d₆) δ 12.08 (s, 1H), 8.65 (s, 1H), 7.82 (d, J=10.4 Hz, 1H), 7.73-7.61 (m, J=6.0 Hz, 2H), 7.41 (t, J=8.0 Hz, 1H), 7.37 (s, 1H), 7.25 (t, J=7.5 Hz, 1H), 7.20-7.14 (m, J=11.0 Hz, 2H), 7.12-6.97 (m, 3H), 5.38 (s, 2H), 3.35 (s, 3H), 2.78 (t, J=7.7 Hz, 2H), 2.44 (t, J=7.7 Hz, 2H) ppm.

### G. Example 33, 3-(3-((3-(3-((6-Fluoro-4-(methylsulfinyl)-1H-indol-5-yl)oxy)phenyl)-1H-1,2,4-triazol-1-yl)methyl)phenyl)propanoic acid

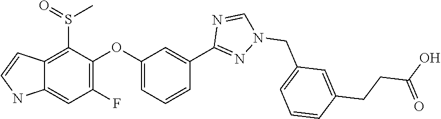 [SMILES: CS(=O)c1c(Oc2cccc(-c3ncn(Cc4cccc(CCC(=O)O)c4)n3)c2)c(F)cc2[nH]ccc12]

To a solution of ethyl 3-(3-((3-(3-((6-fluoro-4-(methylsulfinyl)-1-tosyl-1H-indol-5-yl)oxy)phenyl)-1H-1,2,4-triazol-1-yl)methyl)phenyl)propanoate (90 mg, 0.128 mmol) in water (1 mL) and THF (1 mL) was added lithium hydroxide (30 mg) and heated at 120° C. for 30 minutes in a microwave reactor. The reaction mixture was cooled to 0° C., acidified with 1 N hydrochloric acid to pH˜4 and extracted with ethyl acetate (50 mL). The organic layer was washed with brine (10 mL×2), dried over sodium sulfate, filtered and concentrated. The resulting residue was purified by preparative HPLC to give the title compound, 3-(3-((3-(3-((6-fluoro-4-(methylsulfinyl)-1H-indol-5-yl)oxy)phenyl)-1H-1,2,4-triazol-1-yl)methyl)phenyl)propanoic acid as a white solid (3.7 mg, 5.6%). MS m/z: 519 m/z [M+H]⁺. ¹H NMR (400 MHz, DMSO-d₆) δ 11.85 (s, 1H), 8.65 (s, 1H), 7.69 (d, J=7.7 Hz, 1H), 7.63 (d, J=10.6 Hz, 1H), 7.61-7.58 (m, 1H), 7.43 (t, J=8.1 Hz, 1H), 7.35 (s, 1H), 7.25 (t, J=7.6 Hz, 1H), 7.18 (d, J=11.0 Hz, 2H), 7.12 (s, 1H), 7.07 (d, J=7.7 Hz, 1H), 7.02 (dd, J=7.8, 2.7 Hz, 1H), 5.39 (s, 2H), 2.90 (s, 3H), 2.78 (t, J=7.7 Hz, 2H), 2.46 (s, 3H) ppm.

### Example 34

### 3-(3-((3-(3-((6-Fluoro-4-(methylsulfonyl)-1H-indol-5-yl)oxy)phenyl)-1H-1,2,4-triazol-1-yl)methyl)phenyl)propanamide

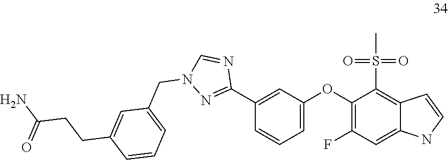 [SMILES: CS(=O)(=O)c1c(Oc2cccc(-c3ncn(Cc4cccc(CCC(N)=O)c4)n3)c2)c(F)cc2[nH]ccc12]

Triethylamine (0.25 mL, 1.8 mmol) was added to a 2 mL microwave vial containing a solution of 3-(3-((3-(3-((6-fluoro-4-(methylsulfonyl)-1H-indol-5-yl)oxy)phenyl)-1H-1,2,4-triazol-1-yl)methyl)phenyl)propanoic acid (Example 32, 32 mg, 0.06 mmol), ammonium chloride (97 mg, 1.8 mmol) and HATU (68 mg, 0.18 mmol) in DMF (2 mL), the vial was sealed and stirred at room temperature for two hours. The reaction mixture was diluted with ethyl acetate (30 mL) and washed with brine (10 mL×2). The organic phase was dried over sodium sulfate, filtered and concentrated. The resulting residue was purified by flash chromatography (0-10% methanol in dichloromethane) twice to afford the title compound (11.1 mg, 35%) as a white solid. LC-MS retention time: 0.97 min. MS: 534 m/z [M+H]⁺.

### Examples 35 and 36

### 3-(3-((2-(3-((6-Fluoro-4-(methylsulfonyl)-1H-indol-5-yl)oxy)phenyl)thiazol-4-yl)methyl)phenyl)propanoic acid

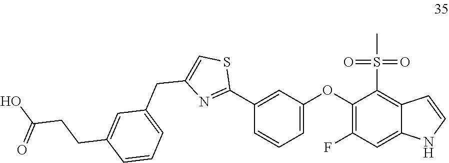 [SMILES: CS(=O)(=O)c1c(Oc2cccc(-c3nc(Cc4cccc(CCC(=O)O)c4)cs3)c2)c(F)cc2[nH]ccc12]

### 3-(3-((2-(3-((6-Fluoro-4-(methylsulfinyl)-1H-indol-5-yl)oxy)phenyl)thiazol-4-yl)methyl)phenyl)propanoic acid

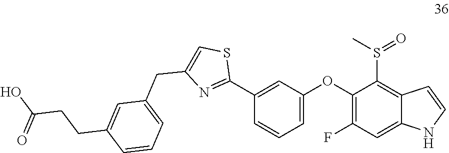 [SMILES: CS(=O)c1c(Oc2cccc(-c3nc(Cc4cccc(CCC(=O)O)c4)cs3)c2)c(F)cc2[nH]ccc12]

### A. Ethyl 3-(3-((2-(3-((6-fluoro-4-(methylthio)-1-tosyl-1H-indol-5-yl)oxy)phenyl)thiazol-4-yl)methyl)phenyl)propanoate

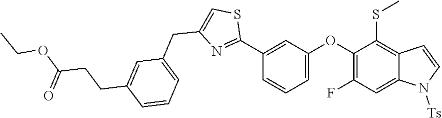 [SMILES: CCOC(=O)CCc1cccc(Cc2csc(-c3cccc(Oc4c(F)cc5c(ccn5S(=O)(=O)c5ccc(C)cc5)c4SC)c3)n2)c1]

To a solution of 3-((6-fluoro-4-(methylthio)-1-tosyl-1H-indol-5-yl)oxy)benzothioamide (Intermediate 33, 280 mg, 75% purity, 0.43 mmol) in ethanol (6 mL) was added ethyl 3-(3-(3-bromo-2-oxopropyl)phenyl)propanoate (Intermediate 21-7, 162 mg, 0.52 mmol) and heated under reflux for three hours. The reaction mixture was cooled to room temperature, diluted with ethyl acetate (100 mL), washed with water and brine, dried over sodium sulfate, filtered and concentrated. The resulting residue was purified by flash chromatography over silica (30%˜60% ethyl acetate in petroleum ether) to give the title compound as a yellow solid (250 mg, 71%). MS: 701 m/z [M+H]⁺. ¹H NMR (400 MHz, CDCl₃) δ 7.85-7.75 (m, 3H), 7.62 (d, J=3.7 Hz, 1H), 7.61-7.55 (m, 2H), 7.31-7.28 (m, 2H), 7.27-7.20 (m, 2H), 7.17-7.03 (m, 3H), 6.89 (d, J=3.7 Hz, 1H), 6.76 (dd, J=8.2, 2.4 Hz, 1H), 6.72 (s, 1H), 4.12-4.08 (m, 2H), 2.93 (t, J=7.8 Hz, 2H), 2.61 (t, J=7.8 Hz, 2H), 2.40 (s, 3H), 2.37 (s, 3H), 1.24-1.18 (m, 3H) ppm. Ethyl 3-(3-((2-(3-((6-fluoro-4-(methylsulfonyl)-1-tosyl-1H-indol-5-yl)oxy)phenyl)thiazol-4-yl)methyl)phenyl)propanoate and ethyl 3-(3-((2-(3-((6-fluoro-4-(methylsulfinyl)-1-tosyl-1H-indol-5-yl)oxy)phenyl)thiazol-4-yl)methyl)phenyl)propanoate.

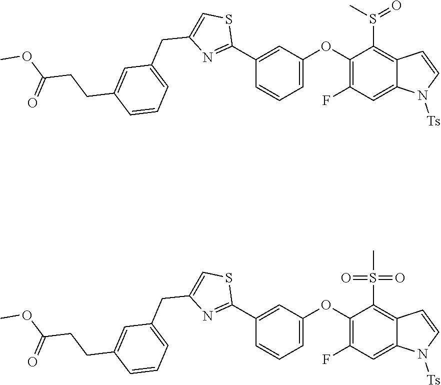 [SMILES: COC(=O)CCc1cccc(Cc2csc(-c3cccc(Oc4c(F)cc5c(ccn5S(=O)(=O)c5ccc(C)cc5)c4S(C)=O)c3)n2)c1 ; COC(=O)CCc1cccc(Cc2csc(-c3cccc(Oc4c(F)cc5c(ccn5S(=O)(=O)c5ccc(C)cc5)c4S(C)(=O)=O)c3)n2)c1]

To a solution of methyl 3-(3-((2-(3-((6-fluoro-4-(methylthio)-1-tosyl-1H-indol-5-yl)oxy)phenyl)thiazol-4-yl)methyl)phenyl)propanoate (310 mg, 86% purity, 0.38 mmol) in methanol (10 mL) was added a solution of ammonium molybdate tetrahydrate (600 mg) in hydrogen peroxide (30% in water, 3 mL) and stirred at room temperature for 2.5 hours. The reaction mixture was diluted with ethyl acetate (50 mL), washed with water (20 mL×2), aqueous sodium sulfite (20 mL) and brine (20 mL×2), dried over sodium sulfate and concentrated. The resulting residue was purified by flash silica gel column chromatography (0-100% ethyl acetate in petroleum ether) and then purified further by preparative HPLC to give ethyl 3-(3-((2-(3-((6-fluoro-4-(methylsulfonyl)-1-tosyl-1H-indol-5-yl)oxy)phenyl)thiazol-4-yl)methyl)phenyl)propanoate (80 mg). MS: 733 m/z [M+H]⁺; and ethyl 3-(3-((2-(3-((6-fluoro-4-(methylsulfinyl)-1-tosyl-1H-indol-5-yl)oxy)phenyl)thiazol-4-yl)methyl)phenyl)propanoate (40 mg). MS: 717 m/z [M+H]⁺. Both isolated as white solids, total yield 43%.

### B. Example 35, 3-(3-((2-(3-((6-Fluoro-4-(methylsulfonyl)-1-tosyl-1H-indol-5-yl)oxy)phenyl)thiazol-4-yl)methyl)phenyl)propanoic acid

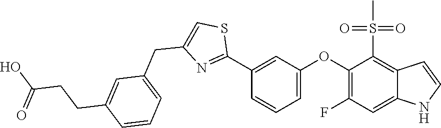 [SMILES: CS(=O)(=O)c1c(Oc2cccc(-c3nc(Cc4cccc(CCC(=O)O)c4)cs3)c2)c(F)cc2[nH]ccc12]

To a solution of ethyl 3-(3-((2-(3-((6-fluoro-4-(methylsulfonyl)-1-tosyl-1H-indol-5-yl)oxy)phenyl)thiazol-4-yl)methyl)phenyl)propanoate (70 mg, 0.096 mmol) in water (1 mL) and THF (1 mL) was added lithium hydroxide (25 mg). The reaction was stirred in a microwave reactor at 120° C. for 30 minutes, cooled to 0° C., acidified with 1 N hydrochloric acid to pH˜4 and extracted with ethyl acetate (30 mL×2). The combined organic extracts were washed with brine (10 mL×2), dried over sodium sulfate, filtered and concentrated. The resulting residue was purified by flash silica gel column chromatography (5% methanol in dichloromethane) to give the title compound, 3-(3-((2-(3-((6-fluoro-4-(methylsulfonyl)-1-tosyl-1H-indol-5-yl)oxy)phenyl)thiazol-4-yl)methyl)phenyl)propanoic acid as a white solid (46.6 mg, 88%). MS: 551 m/z [M+H]⁺. ¹H NMR (400 MHz, DMSO-d₆) δ 12.12 (s, 1H), 11.86 (s, 1H), 7.85 (d, J=10.8 Hz, 1H), 7.68 (t, J=2.8 Hz, 1H), 7.59 (d, J=8.0 Hz, 1H), 7.45-7.42 (m, 2H), 7.28 (s, 1H), 7.21-7.16 (m, 2H), 7.10-7.06 (m, 3H), 7.03-7.00 (m, 1H), 4.06 (s, 2H), 3.38 (s, 3H), 2.78 (t, J=7.6 Hz, 2H), 2.52 (t, J=7.6 Hz, 2H) ppm.

### C. Example 36, 3-(3-((2-(3-((6-Fluoro-4-(methylsulfinyl)-1H-indol-5-yl)oxy)phenyl)thiazol-4-yl)methyl)phenyl)propanoic acid

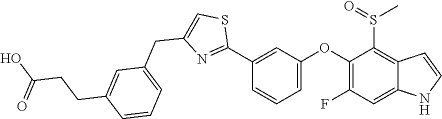 [SMILES: CS(=O)c1c(Oc2cccc(-c3nc(Cc4cccc(CCC(=O)O)c4)cs3)c2)c(F)cc2[nH]ccc12]

The title compound (14.5 mg, 49%, white solid) was similarly prepared according to the procedure for Example 35 using ethyl 3-(3-((2-(3-((6-fluoro-4-(methylsulfinyl)-1-tosyl-1H-indol-5-yl)oxy)phenyl)thiazol-4-yl)methyl)phenyl)propanoate (40 mg, 0.055 mmol). MS m/z: 535 m/z [M+H]⁺. ¹H NMR (400 MHz, CD₃OD) δ 7.62 (d, J=7.7 Hz, 1H), 7.56 (d, J=10.4 Hz, 1H), 7.53 (d, J=3.2 Hz, 1H), 7.49 (s, 1H), 7.44 (t, J=8.0 Hz, 1H), 7.24-7.16 (m, 3H), 7.13-7.08 (m, 2H), 7.07-7.01 (m, 2H), 4.10 (s, 2H), 3.02 (s, 3H), 2.89 (t, J=7.6 Hz, 2H), 2.59 (t, J=7.7 Hz, 2H) ppm.

### Example 37

### 3-(3-(1-(2-(3-((6-Fluoro-4-(methylsulfonyl)-1H-indol-5-yl)oxy)phenyl)thiazol-4-yl)-1-hydroxyethyl)phenyl)propanoic acid

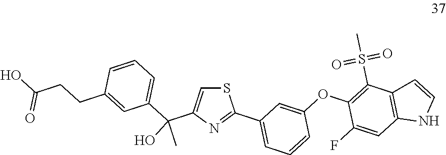 [SMILES: CC(O)(c1cccc(CCC(=O)O)c1)c1csc(-c2cccc(Oc3c(F)cc4[nH]ccc4c3S(C)(=O)=O)c2)n1]

### A. 3-Bromo-1-(3-iodophenyl)propane-1,2-dione

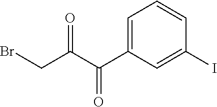 [SMILES: O=C(CBr)C(=O)c1cccc(I)c1]

To a solution of 2-(3-iodophenyl)-2-oxoacetic acid (0.5 g, 0.72 mmol) in dichloromethane (10 mL) was added dropwise a solution of oxalyl chloride (1.14 g, 9 mmol) and a drop of DMF at 0° C. The reaction mixture was warmed to room temperature, stirred for one hour and concentrated to give 2-(3-iodophenyl)-2-oxoacetyl chloride as a yellowish liquid (0.49 g, 93%). MS: 290.7 m/z [M−Cl+CH₃OH]⁺.

To a solution of the above compound (0.49 g, 1.5 mmol) in acetonitrile (5 mL) and THF (5 mL) was added dropwise a 2 M solution of trimethylsilyldiazomethane (1.5 mL, 3 mmol, in hexane) at 0° C. The reaction mixture was warmed to room temperature, stirred for one hour, cooled to 0° C. and a solution of hydrogen bromide in acetic acid (0.75 mL, 4.5 mmol) was added dropwise (vigorous gas evolution noted). After one hour, the reaction mixture was diluted with ethyl acetate (100 mL), washed with water and brine, dried over sodium sulfate, filtered and concentrated. The resulting residue was purified by flash chromatography over silica (petroleum ether/ethyl acetate, v/v, 10/1-5/1) to give 3-bromo-1-(3-iodophenyl)propane-1,2-dione as a yellow liquid (0.17 g, 16%). ¹H NMR (400 MHz, CDCl₃) δ 8.36 (s, 1H), 7.99-7.95 (m, 2H), 7.26 (m, 1H), 3.99 (s, 2H) ppm.

### B. (2-(3-((6-Fluoro-4-(methylsulfonyl)-1H-indol-5-yl)oxy)phenyl)thiazol-4-yl)(3-iodophenyl)methanone

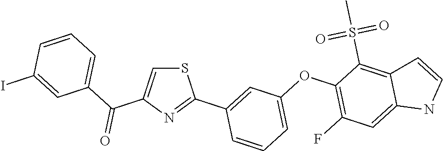

A solution of 3-((6-fluoro-4-(methylsulfonyl)-1H-indol-5-yl)oxy)benzothioamide (Intermediate 33-4, 100 mg, 0.27 mmol) and 3-bromo-1-(3-iodophenyl)propane-1,2-dione (96 mg, 0.27 mmol) in ethanol (2 mL) was refluxed for two hours, cooled to room temperature, diluted with 10 mL of water and extracted with ethyl acetate (10 mL×3). The combined organic extracts were washed with brine (10 mL), dried over sodium sulfate, filtered and concentrated. The resulting residue was purified by flash chromatography (0-70% ethyl acetate in petroleum ether) to give the title compound as a solid (76 mg, 45%).

### C. Ethyl 3-(3-(2-(3-((6-fluoro-4-(methylsulfonyl)-1H-indol-5-yl)oxy)phenyl)thiazole-4-carbonyl)phenyl)propanoate

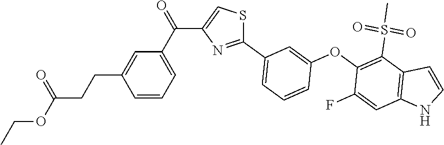 [SMILES: CCOC(=O)CCc1cccc(C(=O)c2csc(-c3cccc(Oc4c(F)cc5[nH]ccc5c4S(C)(=O)=O)c3)n2)c1]

To a solution of (2-(3-((6-fluoro-4-(methylsulfonyl)-1H-indol-5-yl)oxy)phenyl)thiazol-4-yl)(3-iodophenyl)methanone (76 mg, 0.12 mmol), 3,3-diethoxyprop-1-ene (48 mg, 0.36 mmol) and tributylamine (58 μL, 0.24 mmol) in DMF (2 mL) were added tetrabutylammonium chloride (34 mg, 0.12 mmol) and palladium(II) acetate (0.8 mg, 0.0036 mmol) and stirred at 90° C. overnight. The reaction mixture cooled to room temperature, diluted with 2N hydrochloric acid (20 mL), and extracted with ethyl acetate (3×20 mL). The combined organic extracts were washed with brine (10 mL), dried with sodium sulfate and concentrated. The resulting residue was purified by flash chromatography over silica (0-100% ethyl acetate in petroleum ether) to give the title compound as a yellow solid (56 mg, 76%). MS: 593 m/z [M+H]⁺.

### D. 3-(3-(2-(3-((6-Fluoro-4-(methylsulfonyl)-1H-indol-5-yl)oxy)phenyl)thiazole-4-carbonyl)phenyl)propanoic acid

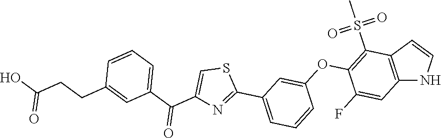 [SMILES: CS(=O)(=O)c1c(Oc2cccc(-c3nc(C(=O)c4cccc(CCC(=O)O)c4)cs3)c2)c(F)cc2[nH]ccc12]

To a solution of ethyl 3-(3-(2-(3-((6-fluoro-4-(methylsulfonyl)-1H-indol-5-yl)oxy)phenyl)thiazole-4-carbonyl)phenyl)propanoate (56 mg, 0.09 mmol) in methanol (1 mL) and THF (3 mL) was added lithium hydroxide (1M in water, 1 mL) and stirred at room temperature for two hours. The reaction mixture was acidified with 1 N hydrochloric acid to pH 6-7, extracted with ethyl acetate (30 mL), washed brine, dried over sodium sulfate, filtered and concentrated. The residue was purified by flash chromatography (0-10% methanol in dichloromethane) to give 3 the title compound as a solid (35 mg, 66%). MS: 565 m/z [M+H]⁺.

### E. Example 37, 3-(3-(1-(2-(3-((6-Fluoro-4-(methylsulfonyl)-1H-indol-5-yl)oxy)phenyl)thiazol-4-yl)-1-hydroxyethyl)phenyl)propanoic acid

Methylmagnesium bromide (0.2 mL, 0.62 mmol, 3M in THF) was added into a solution of 3-(3-(2-(3-((6-fluoro-4-(methylsulfonyl)-1H-indol-5-yl)oxy)phenyl)thiazole-4-carbonyl)phenyl)propanoic acid (35 mg, 0.062 mmol) in dry THF (10 mL) at room temperature under nitrogen atmosphere. The reaction mixture was stirred at room temperature for 30 minutes, quenched with saturated aqueous ammonium chloride and extracted with ethyl acetate (10 mL×3). The aqueous layer was acidified with 2N hydrochloric acid to pH 2-3 and extracted with dichloromethane. The organic layer was washed with brine, dried over sodium sulfate, filtered and concentrated to give the crude product which was purified by preparative HPLC to give the title compound as white solid (3.5 mg, 10%). MS: 581 m/z [M+H]⁺. ¹H NMR (400 MHz, CD₃OD) δ 7.62-7.69 (m, 2H), 7.54-7.55 (m, 2H), 7.30-7.41 (m, 4H), 7.09-7.22 (m, 3H), 6.97-6.99 (m, 1H), 3.32 (s, 3H), 2.87-2.92 (t, 2H), 2.53-2.57 (m, 2H), 1.94 (s, 3H) ppm.

### Examples 38 and 39

### 3-(3-(1-(3-(2-Fluoro-5-((6-fluoro-4-(methylsulfonyl)-1H-indol-5-yl)oxy)phenyl)-1H-pyrazol-1-yl)ethyl)phenyl)propanoic acid

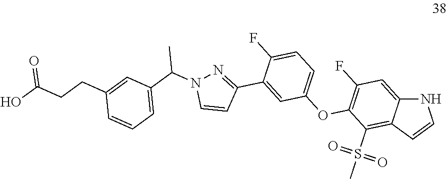 [SMILES: CC(c1cccc(CCC(=O)O)c1)n1ccc(-c2cc(Oc3c(F)cc4[nH]ccc4c3S(C)(=O)=O)ccc2F)n1]

### 3-(3-(1-(5-(2-Fluoro-5-((6-fluoro-4-(methylsulfonyl)-1H-indol-5-yl)oxy)phenyl)-1H-pyrazol-1-yl)ethyl)phenyl)propanoic acid

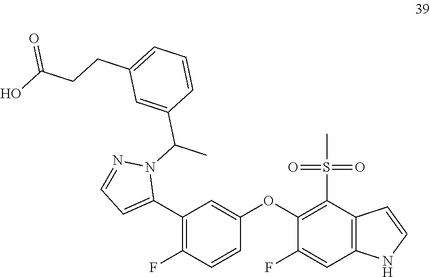 [SMILES: CC(c1cccc(CCC(=O)O)c1)n1nccc1-c1cc(Oc2c(F)cc3[nH]ccc3c2S(C)(=O)=O)ccc1F]

### A. Ethyl 3-(3-acetylphenyl)propanoate

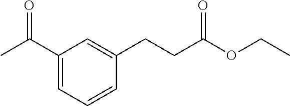 [SMILES: CCOC(=O)CCc1cccc(C(C)=O)c1]

To a solution of 1-(3-iodophenyl)ethan-1-one (6.20 g, 25.3 mmol), 3,3-diethoxyprop-1-ene (10.0 g, 75.8 mmol) and tributylamine (9.4 g, 51 mmol) in DMF (20 mL) was added tetrabutylammonium chloride (7.02 g, 25.3 mmol) and palladium(II) acetate (170 mg, 0.760 mmol). The reaction was heated overnight at 90° C. The mixture was then cooled to room temperature, diluted with 2N hydrochloric acid (200 mL) and extracted with ethyl acetate (3×100 mL). The combined organic extracts were washed with water and brine (50 mL), dried over sodium sulfate and concentrated. The resulting residue was purified by flash chromatography over silica (0-50% ethyl acetate in petroleum ether) to afford the title compound as a yellow solid (3.20 g, 60%). MS: 221 m/z [M+H]⁺.

### B. Ethyl 3-(3-(1-hydroxyethyl)phenyl)propanoate

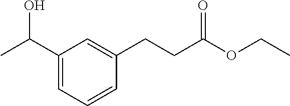 [SMILES: CCOC(=O)CCc1cccc(C(C)O)c1]

To a stirred and cooled (0° C.) solution of step A product (5.00 g, 22.7 mmol) in ethanol (30 mL) was added sodium borohydride (2.60 g, 68.0 mmol), portionwise over 4-5 minutes. The reaction was stirred at room temperature for three hours. The reaction was then quenched with water (10 mL), stirred for five minutes and concentrated. The residue was dissolved in ethyl acetate (150 mL) and the resulting solution was washed with water (30 mL×2) and brine (30 mL×2), dried over sodium sulfate and concentrated to afford the title compound as a light yellow oil (4.13 g, 82%). MS: 223 m/z [M+H]⁺.

### C. Ethyl 3-(3-(1-bromoethyl)phenyl)propanoate

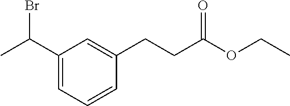 [SMILES: CCOC(=O)CCc1cccc(C(C)Br)c1]

To a stirred and cooled (0° C.) solution of step B product (1.11 g, 4.99 mmol) in dichloromethane (30 mL) was added N-bromosuccinimide (1.20 g, 6.74 mmol) and PPh₃ (2.00 g, 7.63 mmol). The reaction mixture was stirred at room temperature overnight and then diluted with dichloromethane (100 mL). This solution was washed with water (50 mL×3) and brine (50 mL×2), dried over sodium sulfate and concentrated. The resulting residue was purified by flash chromatography over silica (0-30% ethyl acetate in petroleum ether) to afford the title compound as a light yellow solid (560 mg, 36%). MS: 285, 287 m/z [M+H]⁺.

### D. Ethyl 3-(3-(1-(3-(2-fluoro-5-((6-fluoro-4-(methylthio)-1-tosyl-1H-indol-5-yl)oxy)phenyl)-1H-pyrazol-1-yl)ethyl)phenyl)propanoate and ethyl 3-(3-(1-(5-(2-fluoro-5-((6-fluoro-4-(methylthio)-1-tosyl-1H-indol-5-yl)oxy)phenyl)-1H-pyrazol-1-yl)ethyl)phenyl)propanoate

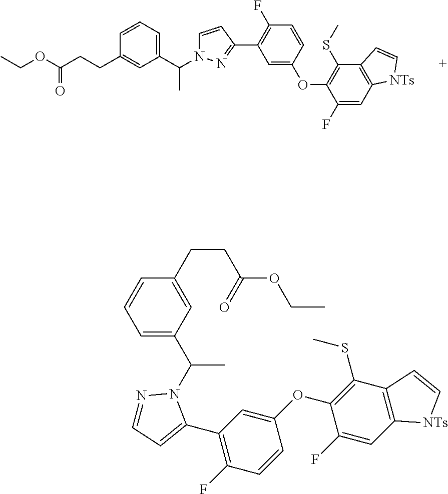 [SMILES: CCOC(=O)CCc1cccc(C(C)n2ccc(-c3cc(Oc4c(F)cc5c(ccn5S(=O)(=O)c5ccc(C)cc5)c4SC)ccc3F)n2)c1 ; CCOC(=O)CCc1cccc(C(C)n2nccc2-c2cc(Oc3c(F)cc4c(ccn4S(=O)(=O)c4ccc(C)cc4)c3SC)ccc2F)c1]

To a stirred solution of 6-fluoro-5-(4-fluoro-3-(1H-pyrazol-3-yl)phenoxy)-4-(methylthio)-1-tosyl-1H-indole (240 mg, 0.470 mmol, Intermediate 34) and step C product (200 mg, 0.701 mmol) in acetonitrile (20 mL) was added cesium carbonate (305 mg, 0.936 mmol). The reaction mixture was heated at 80° C. for three hours, cooled to room temperature and filtered. The solids were rinsed with acetonitrile (5 mL×3) and the combined filtrate was concentrated. The residue was purified by flash chromatography over silica (0-30% ethyl acetate in petroleum ether) to give a mixture of the title compounds as a solid (290 mg, 86%). MS: 716 m/z [M+H]⁺.

### E. Ethyl 3-(3-(1-(3-(2-fluoro-5-((6-fluoro-4-(methylsulfonyl)-1-tosyl-1H-indol-5-yl)oxy)phenyl)-1H-pyrazol-1-yl)ethyl)phenyl)propanoate and ethyl 3-(3-(1-(5-(2-fluoro-5-((6-fluoro-4-(methylsulfonyl)-1-tosyl-1H-indol-5-yl)oxy)phenyl)-1H-pyrazol-1-yl)ethyl)phenyl)propanoate

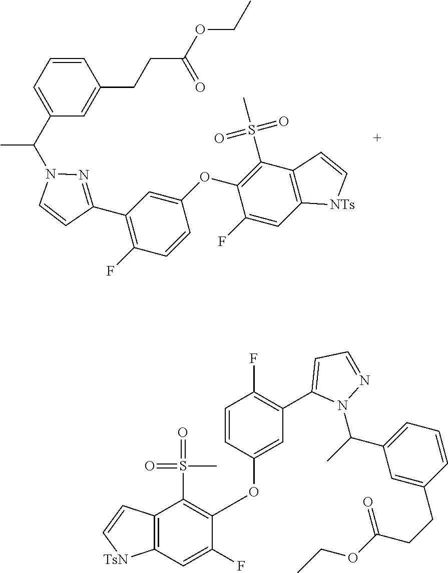 [SMILES: CCOC(=O)CCc1cccc(C(C)n2ccc(-c3cc(Oc4c(F)cc5c(ccn5S(=O)(=O)c5ccc(C)cc5)c4S(C)(=O)=O)ccc3F)n2)c1 ; CCOC(=O)CCc1cccc(C(C)n2nccc2-c2cc(Oc3c(F)cc4c(ccn4S(=O)(=O)c4ccc(C)cc4)c3S(C)(=O)=O)ccc2F)c1]

To a stirred solution of step D product (290 mg, 0.405 mmol) in methanol (20 mL) was added a solution of ammonium molybdate tetrahydrate (600 mg, 0.485 mmol) in 30% aqueous hydrogen peroxide (3 mL). The reaction was stirred at room temperature for four hours and then diluted with ethyl acetate (80 mL). The solution was washed with water (30 mL×3), aqueous sodium sulfite solution (20 mL) and brine (20 mL×2), dried and concentrated. The resulting residue was purified by flash chromatography over silica (0-30% ethyl acetate in petroleum ether) to afford a mixture of title compounds as a solid (0.270 g, 89%). MS: 748 m/z [M+H]⁺ (for both product isomers).

### F. Example 38, 3-(3-(1-(3-(2-Fluoro-5-((6-fluoro-4-(methylsulfonyl)-1H-indol-5-yl)oxy)phenyl)-1H-pyrazol-1-yl)ethyl)phenyl)propanoic acid and Example 39, 3-(3-(1-(5-(2-Fluoro-5-((6-fluoro-4-(methylsulfonyl)-1-tosyl-1H-indol-5-yl)oxy)phenyl)-1H-pyrazol-1-yl)ethyl)phenyl)propanoic acid

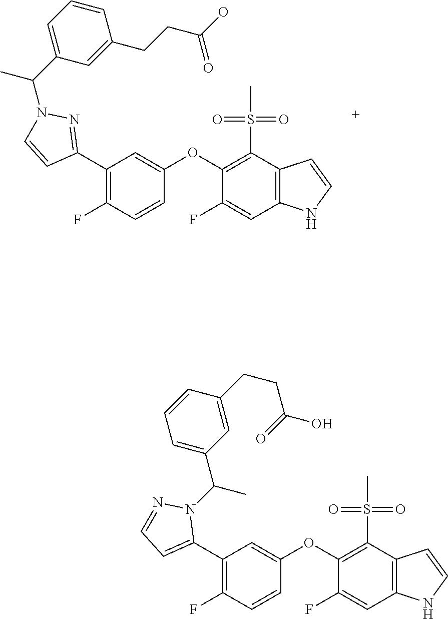 [SMILES: CC(c1cccc(CCC(=O)O)c1)n1ccc(-c2cc(Oc3c(F)cc4[nH]ccc4c3S(C)(=O)=O)ccc2F)n1 ; CC(c1cccc(CCC(=O)O)c1)n1nccc1-c1cc(Oc2c(F)cc3[nH]ccc3c2S(C)(=O)=O)ccc1F]

To a stirred solution of step E product (150 mg, 0.201 mmol) in 1:1 THF/water (4 mL) was added lithium hydroxide monohydrate (40.0 mg, 0.953 mmol). The reaction was heated for 30 minutes at 120° C. in microwave reactor and then cooled and acidified by the addition of sufficient 1N hydrochloric acid to achieve ˜pH 4. The suspension was taken up in ethyl acetate (50 mL) and this solution was washed with brine (10 mL×3), dried over sodium sulfate and concentrated. The residue was purified by preparative HPLC to afford the separate, purified product isomers:

Example 38 was obtained as a solid (47.8 mg, 42%). ¹H NMR (400 MHz, CD₃OD) δ 7.72 (d, J=2.4 Hz, 1H), 7.66 (d, J=10.5 Hz, 1H), 7.56-7.51 (m, 2H), 7.26-7.07 (m, 6H), 6.92-6.78 (m, 1H), 6.71 (dd, J=4.0, 2.4 Hz, 1H), 5.59 (q, J=7.1 Hz, 1H), 3.33 (s, 3H), 2.89 (t, J=7.6 Hz, 2H), 2.58 (t, J=7.7 Hz, 2H), 1.88 (d, J=7.1 Hz, 3H) ppm. MS: 566 m/z [M+H]⁺.

Example 39 was obtained as a solid (6.5 mg, 6%). ¹H NMR (400 MHz, CD₃OD) δ 7.62 (d, J=2.0 Hz, 1H), 7.55 (d, J=7.6 Hz, 1H), 7.53 (s, 1H), 7.25-7.12 (d, J=8.8 Hz, 1H), 7.20-7.18 (m, 1H), 7.15-7.11 (m, 1H), 6.98 (t, J=7.6 Hz, 1H), 6.90 (d, J=7.8 Hz, 1H), 6.76 (d, J=8.0 Hz, 1H), 6.73 (s, 1H), 6.63-6.61 (dd, J=5.2, 2.8 Hz, 1H), 6.43 (s, 1H), 5.42 (q, J=6.8 Hz, 1H), 3.33 (s, 3H), 2.75 (t, J=7.6 Hz, 2H), 2.43 (t, J=7.6 Hz, 2H), 1.81 (d, J=6.8 Hz, 3H) ppm. MS: 566 m/z [M+H]⁺.

The following Example compound was prepared by a synthetic route analogous to that described for Examples 38 and 39.

| | | | | LCMS | LCMS |
|---|---|---|---|---|---|
| Structure | Ex. No. | Name | ¹H NMR | Rₜ (min) | (m/z) |
| 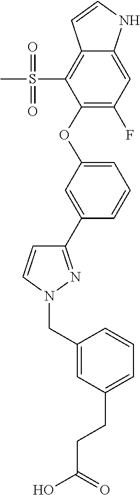 [SMILES: CS(=O)(=O)c1c(Oc2cccc(-c3ccn(Cc4cccc(CCC(=O)O)c4)n3)c2)c(F)cc2[nH]ccc12] | 40 | 3-(3-((3-(3-((6-fluoro-4- (methylsulfonyl)-1H-indol-5- yl)oxy)phenyl)-1H-pyrazol-1- yl)methyl)phenyl)propanoic acid | (400 MHz, CD₃OD) δ 7.66 (d, J = 4.2 Hz, 1H),7.64 (s, 1H), 7.52 (d, J = 3.2 Hz, 1H), 7.50 (d, J = 8.0 Hz, 1H), 7.41 (s, 1H), 7.35 (t, J = 8.4 Hz, 1H), 7.27 (t, J = 7.6 Hz, 1H), 7.21-7.16 (m, 3H), 7.07 (d, J = 7.6 Hz, 1H), 6.87 (dd, J = 8.4, 2.4 Hz, 1H), 6.64 (d, J = 2.8 Hz, 1H), 5.33 (s, 2H), 3.33 (s, 3H), 2.90 (t, J = 8.0 Hz, 2H), 2.59 (t, J = 8.0 Hz, 2H) ppm. | 1.26 | 534 [M + H]⁺ |

### Example 41

### Enantiomer 1 of 3-(2-fluoro-3-(1-(3-(2-fluoro-5-((6-fluoro-4-(methylsulfonyl)-1H-indol-5-yl)oxy)phenyl)-1H-pyrazol-1-yl)ethyl)phenyl)propanoic acid (41A)

### And

### Enantiomer 2 of 3-(2-fluoro-3-(1-(3-(2-fluoro-5-((6-fluoro-4-(methylsulfonyl)-1H-indol-5-yl)oxy)phenyl)-1H-pyrazol-1-yl)ethyl)phenyl)propanoic acid (41B)

### Absolute Configurations Unknown

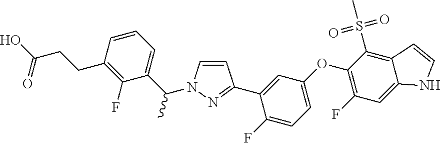 [SMILES: CC(c1cccc(CCC(=O)O)c1F)n1ccc(-c2cc(Oc3c(F)cc4[nH]ccc4c3S(C)(=O)=O)ccc2F)n1]

### A. 1-Bromo-3-(1-bromoethyl)-2-fluorobenzene

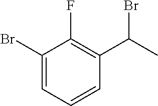

To a stirred and cooled (0° C.) solution of 1-(3-bromo-2-fluorophenyl)ethan-1-ol (2.80 g, 12.8 mmol) and pyridine (2.02 g, 25.6 mmol) in dichloromethane (50 mL) was added, dropwise, a solution of PBr₃ (25.6 mmol, 6.92 g) in dichloromethane (10 mL). The reaction was stirred at 0° C. for 30 min, then allowed to warm to room temperature and stirred for another four hours. The mixture was then poured into water (20 mL) and extracted with ethyl acetate (20 mL×3). The combined extracts were dried over sodium sulfate and concentrated. The residue was purified by flash chromatography over silica (0-5% ethyl acetate in petroleum ether) to afford the title compound as a colorless oil (2.00 g, 56%). ¹H NMR (400 MHz, CDCl₃) δ 7.53-7.45 (m, 2H), 7.05 (m, 1H), 5.45 (q, J=7.0 Hz, 1H), 2.04 (d, J=7.0 Hz, 3H) ppm.

### B. 5-(3-(1-(1-(3-Bromo-2-fluorophenyl)ethyl)-1H-pyrazol-3-yl)-4-fluorophenoxy)-6-fluoro-4-(methylthio)-1-tosyl-1H-indole

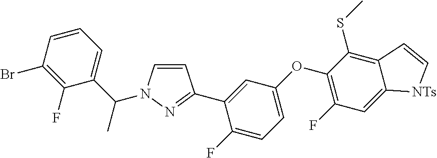 [SMILES: CSc1c(Oc2ccc(F)c(-c3ccn(C(C)c4cccc(Br)c4F)n3)c2)c(F)cc2c1ccn2S(=O)(=O)c1ccc(C)cc1]

Exchanging ethyl 3-(3-(1-bromoethyl)phenyl)propanoate for the step A product (1.85 g, 6.57 mmol), the reaction conditions described for Example 38 Step D were used to prepare the title compound as a yellow oil (4.40 g, 88%). MS: 712, 714 m/z [M+H]⁺.

### C. 5-(3-(1-(1-(3-Bromo-2-fluorophenyl)ethyl)-1H-pyrazol-3-yl)-4-fluorophenoxy)-6-fluoro-4-(methylthio)-1H-indole

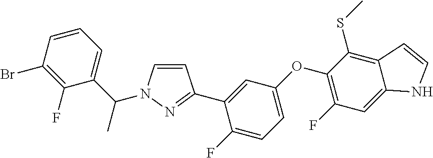 [SMILES: CSc1c(Oc2ccc(F)c(-c3ccn(C(C)c4cccc(Br)c4F)n3)c2)c(F)cc2[nH]ccc12]

To a stirred solution of step B product (3.00 g, 4.21 mmol) in 5:1:1 THF/methanol/water (42 ml) was added lithium hydroxide monohydrate (707 mg, 16.8 mmol). The reaction was stirred at room temperature overnight and then diluted with ethyl acetate (150 mL). This solution was washed with water (30 mL) and brine (30 mL), dried over sodium sulfate and concentrated. The residue was purified by flash chromatography over silica (0-30% ethyl acetate in petroleum ether) to afford the title compound as a yellow oil (2.30 g, 98%). MS: 558, 560 m/z [M+H]⁺.

### D. Ethyl 3-(2-fluoro-3-(1-(3-(2-fluoro-5-((6-fluoro-4-(methylthio)-1H-indol-5-yl)oxy)phenyl)-1H-pyrazol-1-yl)ethyl)phenyl)propanoate

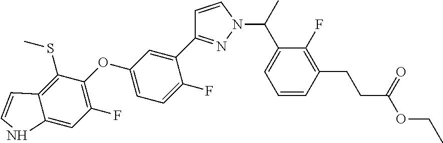 [SMILES: CCOC(=O)CCc1cccc(C(C)n2ccc(-c3cc(Oc4c(F)cc5[nH]ccc5c4SC)ccc3F)n2)c1F]

Exchanging 3-Iodophenylacetic acid for the step C product (2.30 g, 4.12 mmol), the same two-step reaction sequence used to prepare Intermediate 21A and convert it to Intermediate 21B was used to prepare the title compound as a yellow oil (0.550 g, 23%). MS: 580 m/z [M+H]⁺.

### E. Enantiomer 1 and 2 of ethyl 3-(2-fluoro-3-(1-(3-(2-fluoro-5-((6-fluoro-4-(methylsulfonyl)-1H-indol-5-yl)oxy)phenyl)-1H-pyrazol-1-yl)ethyl)phenyl)propanoate

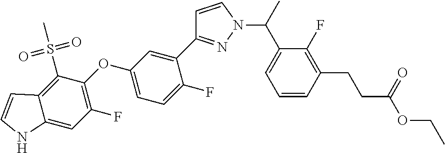 [SMILES: CCOC(=O)CCc1cccc(C(C)n2ccc(-c3cc(Oc4c(F)cc5[nH]ccc5c4S(C)(=O)=O)ccc3F)n2)c1F]

Exchanging ethyl 3-(3-(1-(3-(2-fluoro-5-((6-fluoro-4-(methylthio)-1-tosyl-1H-indol-5-yl)oxy)phenyl)-1H-pyrazol-1-yl)ethyl)phenyl)propanoate for step D product (0.280 g, 0.483 mmol), the reaction conditions described in step E of the Example 38 synthesis were used to prepare the racemic title compound as a colorless oil (0.220 g, 74%). This material was separated into its component enantiomers by chiral SFC (Thar Prep 80 system; 20×250 mm×10 μm CHIRALCEL OJ column; flow rate: 80 mL/min; eluant: 70:30 methanol/CO₂ with 0.2% ammonia/methanol additive).

Enantiomer 1 (first eluting isomer) of the title compound was obtained as a colorless oil (0.110 g, 37%). MS: 612 m/z [M+H]⁺.

Enantiomer 2 (second eluting isomer) of the title compound was obtained as a colorless oil (0.095 g, 32%). MS: 612 m/z [M+H]⁺.

### F. Enantiomers 1 and 2 of 3-(2-fluoro-3-(1-(3-(2-fluoro-5-((6-fluoro-4-(methylsulfonyl)-1H-indol-5-yl)oxy)phenyl)-1H-pyrazol-1-yl)ethyl)phenyl)propanoic acid (Absolute Configurations Unknown)

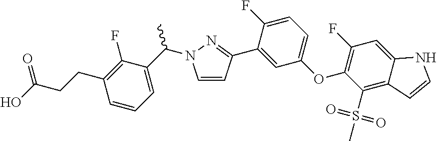 [SMILES: CC(c1cccc(CCC(=O)O)c1F)n1ccc(-c2cc(Oc3c(F)cc4[nH]ccc4c3S(C)(=O)=O)ccc2F)n1]

Exchanging ethyl 3-(3-(2-(3-((6-fluoro-4-(methylsulfonyl)-1H-indol-5-yl)oxy)phenyl)thiazole-4-carbonyl)phenyl)propanoate for the separate step E products, Enantiomer 1 (0.110 g, 0.180 mmol) and Enantiomer 2 (0.095 g, 0.155 mmol), the reaction conditions described in step D of Example 37 were used to prepare the title compounds (Example 41A and Example 41B, respectively).

Example 41A (enantiomer 1) was obtained as a white solid (55.8 mg, 52%, 98% ee). ¹H NMR (400 MHz, CD₃OD) δ 7.77 (d, J=2.0 Hz, 1H), 7.65 (d, J=10.4 Hz, 1H), 7.53 (m, 2H), 7.30-6.94 (m, 5H), 6.88-6.80 (m, 1H), 6.74-6.69 (m, 1H), 5.92 (q, J=14.4, 7.2 Hz, 1H), 3.32-3.31 (m, 3H), 2.96 (t, J=7.8 Hz, 2H), 2.61 (t, J=7.8 Hz, 2H), 1.89 (d, J=7.2 Hz, 3H) ppm. MS: 584 m/z [M+H]⁺.

Example 41B (enantiomer 2) was obtained as a white solid (57.1 mg, 59%, 98% ee). ¹H NMR (400 MHz, CD₃OD) 7.77 (d, J=2.4 Hz, 1H), 7.65 (d, J=10.4 Hz, 1H), 7.52 (m, 2H), 7.29-7.18 (m, 2H), 7.15-6.92 (m, 3H), 6.84 (m, 1H), 6.72 (m, 1H), 5.92 (q, J=14.4, 7.2 Hz, 1H), 3.32-3.29 (m, 3H), 2.96 (t, J=7.6 Hz, 2H), 2.61 (t, J=7.6 Hz, 2H), 1.89 (d, J=7.2 Hz, 3H) ppm. MS: 584 m/z [M+H]⁺.

### Example 42

### 3-(3-((2-(3-((6-Fluoro-4-(methylsulfonyl)-1H-indol-5-yl)oxy)phenyl)oxazol-4-yl)methyl)phenyl)propanoic acid

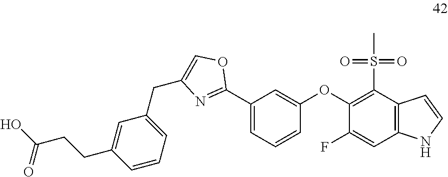 [SMILES: CS(=O)(=O)c1c(Oc2cccc(-c3nc(Cc4cccc(CCC(=O)O)c4)co3)c2)c(F)cc2[nH]ccc12]

### A. 3-((6-Fluoro-4-(methylthio)-1H-indol-5-yl)oxy)benzoic acid

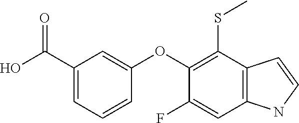 [SMILES: CSc1c(Oc2cccc(C(=O)O)c2)c(F)cc2[nH]ccc12]

To a stirred solution of 3-((6-fluoro-4-(methylthio)-1-tosyl-1H-indol-5-yl)oxy)benzonitrile (1.00 g, 2.21 mmol, Intermediate 23C-1) in water (40 mL) was added potassium hydroxide (1.23 g, 21.9 mmol). The reaction was heated at reflux overnight and then cooled to room temperature and brought to ˜pH 4 with the addition of 2N hydrochloric acid. The resulting suspension was extracted with ethyl acetate (70 mL). The organic phase was washed with brine (10 mL×2), dried over sodium sulfate and concentrated. The crude product was purified by flash chromatography over silica (0-35% ethyl acetate in petroleum ether) to afford the title compound as a solid (0.700 g, 100%). ¹H NMR (400 MHz, CDCl₃) δ 8.35 (s, 1H), 7.76 (dt, J=7.6, 1.2 Hz, 1H), 7.56-7.55 (m, 1H), 7.38 (t, J=8.0 Hz, 1H), 7.29 (dd, J=3.2, 2.8 Hz, 1H), 7.21-7.18 (m, 2H), 6.78-6.76 (m, 1H), 2.46 (s, 3H) ppm. MS: 316 m/z [M−H]⁻.

### B. 3-((6-Fluoro-4-(methylthio)-1-tosyl-1H-indol-5-yl)oxy)benzoic acid

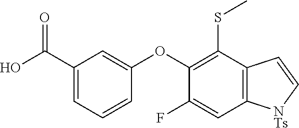 [SMILES: CSc1c(Oc2cccc(C(=O)O)c2)c(F)cc2c1ccn2S(=O)(=O)c1ccc(C)cc1]

To a stirred and cooled (0° C.) solution of step product (0.700 g, 2.21 mmol) in THF (20 mL) was added a 60% dispersion of sodium hydride in mineral oil (0.203 g, 5.08 mmol). After another 30 minutes, the mixture was treated with TsCl (0.545 g, 2.87 mmol) and the cooling bath was removed. The reaction mixture was stirred at room temperature overnight and then partitioned between water (30 mL) and ethyl acetate (20 mL). The organic layer was combined with additional extracts (ethyl acetate, 2×20 mL), washed with brine (10 mL), dried over sodium sulfate and concentrated. The resulting residue was purified by flash chromatography over silica (0-40% ethyl acetate in petroleum ether) to afford the title compound as a solid (0.850 g, 85%). ¹H NMR (400 MHz, CDCl₃) δ 7.83-7.74 (m, 4H), 7.63 (d, J=3.6 Hz, 1H), 7.50 (t, J=2.0 Hz, 1H), 7.38 (t, J=8.0 Hz, 1H), 7.30 (d, J=8.0 Hz, 2H), 7.12 (dd, J=8.4, 2.8 Hz, 1H), 6.88 (d, J=3.6 Hz, 1H), 2.39 (s, 3H), 2.38 (s, 3H) ppm. MS: 470 m/z [M−H]⁻.

### C. 3-(3-(3-Ethoxy-3-oxopropyl)phenyl)-2-oxopropyl 3-((6-fluoro-4-(methylthio)-1-tosyl-1H-indol-5-yl)oxy)benzoate

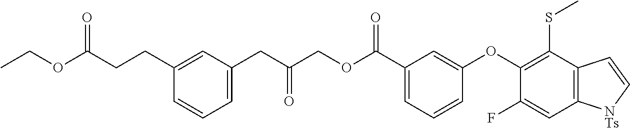 [SMILES: CCOC(=O)CCc1cccc(CC(=O)COC(=O)c2cccc(Oc3c(F)cc4c(ccn4S(=O)(=O)c4ccc(C)cc4)c3SC)c2)c1]

To a stirred solution of step B product (0.750 g, 1.59 mmol) in dichloromethane (20 mL) was added ethyl 3-(3-(3-bromo-2-oxopropyl)phenyl)propanoate (0.647 g, 2.07 mmol, Intermediate 21-7) and triethylamine (446 μL, 3.18 mmol). The reaction was stirred at room temperature overnight and then partitioned between water (20 mL) and ethyl acetate (30 mL). The organic layer was combined with additional extracts (ethyl acetate, 2×30 mL), washed with brine (1×30 mL), dried over sodium sulfate and concentrated. The residue was purified by flash chromatography over silica (50% ethyl acetate in petroleum ether) to afford the title compound as a solid (0.550 g, 49%). ¹H NMR (400 MHz, CDCl₃) δ 7.81-7.79 (m, 3H), 7.75 (dt, J=8.0, 0.8 Hz, 1H), 7.62 (d, J=3.6 Hz, 1H), 7.54-7.53 (m, 1H), 7.36 (t, J=8.0 Hz, 1H), 7.31-7.24 (m, 3H), 7.12 (d, J=8.0 Hz, 1H), 7.07-7.05 (m, 3H), 6.87 (d, J=3.6 Hz, 1H), 4.87 (s, 2H), 4.12 (q, J=7.2 Hz, 2H), 3.75 (s, 2H), 2.92 (t, J=8.0 Hz, 2H), 2.60 (t, J=8.0 Hz, 2H), 2.39 (s, 3H), 2.38 (s, 3H), 1.22 (t, J=7.2 Hz, 3H) ppm. MS: 726.7 m/z [M+Na]⁺.

### D. Ethyl 3-(3-((2-(3-((6-fluoro-4-(methylthio)-1-tosyl-1H-indol-5-yl)oxy)phenyl)oxazol-4-yl)methyl)phenyl)propanoate

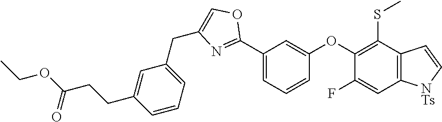 [SMILES: CCOC(=O)CCc1cccc(Cc2coc(-c3cccc(Oc4c(F)cc5c(ccn5S(=O)(=O)c5ccc(C)cc5)c4SC)c3)n2)c1]

To a stirred solution of step C product (0.550 g, 0.781 mmol) in acetic acid (10 mL) was added ammonium acetate (0.301 g, 3.9 mmol). The mixture was heated at reflux overnight and then cooled and concentrated. The residue was purified by flash chromatography over silica (0-40% ethyl acetate in petroleum ether) to afford the title compound as a solid (0.134 g, 24%). MS: 685 m/z [M+H]⁺.

### E. Ethyl 3-(3-((2-(3-((6-fluoro-4-(methylsulfonyl)-1-tosyl-1H-indol-5-yl)oxy)phenyl)oxazol-4-yl)methyl)phenyl)propanoate

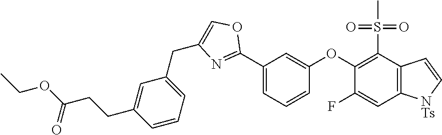 [SMILES: CCOC(=O)CCc1cccc(Cc2coc(-c3cccc(Oc4c(F)cc5c(ccn5S(=O)(=O)c5ccc(C)cc5)c4S(C)(=O)=O)c3)n2)c1]

To a stirred solution of step D product (0.134 g, 0.196 mmol) in 1:1 methanol/THF (14 mL) was added a solution of ammonium molybdate tetrahydrate (0.268 g, 0.217 mmol) in 30% aqueous hydrogen peroxide (1.4 mL). The reaction was and stirred for three hours at room temperature and then partitioned between ethyl acetate (70 mL) and water (20 mL). The organic layer was washed with additional water (3×20 mL) and brine (2×10 mL), dried over sodium sulfate and concentrated. The residue was purified by flash chromatography over silica (0-60% ethyl acetate in petroleum ether) to afford the title compound as a solid (0.085 g, 60%). MS: 717 m/z [M+H]⁺.

### F. Example 42, 3-(3-((2-(3-((6-Fluoro-4-(methylsulfonyl)-1H-indol-5-yl)oxy)phenyl)oxazol-4-yl)methyl)phenyl)propanoic acid

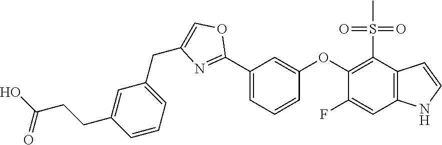 [SMILES: CS(=O)(=O)c1c(Oc2cccc(-c3nc(Cc4cccc(CCC(=O)O)c4)co3)c2)c(F)cc2[nH]ccc12]

To a stirred solution of step E product (70.0 mg, 97.7 μmol) in 1:1 THF/water (4 mL) was added lithium hydroxide monohydrate (35.0 mg, 0.486 mmol). The reaction was heated for 1.5 hours at 100° C. in a microwave reactor and then cooled and made acidic (˜pH 4) with the addition of 1N hydrochloric acid. The suspension was partitioned between ethyl acetate (50 mL) and brine (50 mL). The organic layer was dried over sodium sulfate and concentrated to afford a residue which was purified by flash chromatography over silica (0-95% ethyl acetate in petroleum ether). The title compound was obtained as a white solid (31.1 mg, 60%). ¹H NMR (400 MHz, CD₃OD) δ 7.72 (d, J=8.4 Hz, 1H), 7.68 (d, J=10.4 Hz, 1H), 7.59 (s, 1H), 7.54 (d, J=3.2 Hz, 1H), 7.51 (s, 1H), 7.47 (t, J=8.0 Hz, 1H), 7.23-7.21 (m, 2H), 7.18 (s, 1H), 7.13-7.11 (m, 3H), 3.88 (s, 2H), 3.32 (s, 3H), 2.90 (t, J=7.6 Hz, 2H), 2.59 (t, J=7.6 Hz, 2H) ppm. MS: 535 m/z [M+H]⁺.

### Example 43

### Enantiomer 1 of 3-(2-fluoro-3-(1-(2-(2-fluoro-5-((6-fluoro-4-(methylsulfonyl)-1H-indol-5-yl)oxy)phenyl)oxazol-4-yl)ethyl)phenyl)propanoic acid (43A)

### Enantiomer 2 of 3-(2-fluoro-3-(1-(2-(2-fluoro-5-((6-fluoro-4-(methylsulfonyl)-1H-indol-5-yl)oxy)phenyl)oxazol-4-yl)ethyl)phenyl)propanoic acid (43B)

### Absolute Configurations Unknown

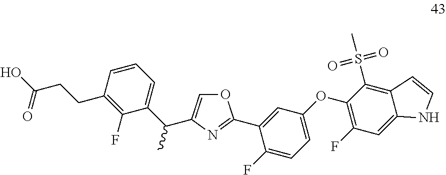 [SMILES: CC(c1coc(-c2cc(Oc3c(F)cc4[nH]ccc4c3S(C)(=O)=O)ccc2F)n1)c1cccc(CCC(=O)O)c1F]

### A. Ethyl 3-[2-fluoro-3-[1-[2-[2-fluoro-5-[(6-fluoro-4-methylsulfonyl-1H-indol-5-yl)oxy]phenyl]oxazol-4-yl]ethyl]phenyl]propanoate

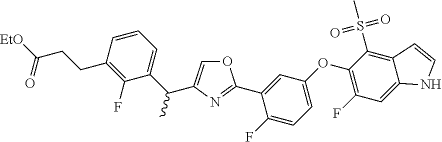 [SMILES: CCOC(=O)CCc1cccc(C(C)c2coc(-c3cc(Oc4c(F)cc5[nH]ccc5c4S(C)(=O)=O)ccc3F)n2)c1F]

Exchanging 3-((6-fluoro-4-(methylthio)-1-tosyl-1H-indol-5-yl)oxy)benzonitrile (Intermediate 23C-1) for 2-fluoro-5-((6-fluoro-4-(methylthio)-1-tosyl-1H-indol-5-yl)oxy)benzonitrile (intermediate 23C) and ethyl 3-(3-(3-bromo-2-oxopropyl)phenyl)propanoate (Intermediate 21-7) for ethyl 3-(3-(4-chloro-3-oxobutan-2-yl)-2-fluorophenyl)propanoate, the five-step reaction sequence described in steps A-E of Example 42, was used to prepare the title compound as a colorless oil (0.170 g). MS: 613 m/z [M+H]⁺.

### B. Enantiomers of 3-(2-fluoro-3-(1-(2-(2-fluoro-5-((6-fluoro-4-(methylsulfonyl)-1H-indol-5-yl)oxy)phenyl)oxazol-4-yl)ethyl)phenyl)propanoic acid (Absolute Configuration Unknown)

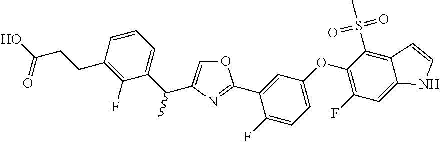 [SMILES: CC(c1coc(-c2cc(Oc3c(F)cc4[nH]ccc4c3S(C)(=O)=O)ccc2F)n1)c1cccc(CCC(=O)O)c1F]

To a stirred solution of step A product (0.170 g, 0.277 mmol) in 1:1 THF/water (4 mL) was added lithium hydroxide monohydrate (0.117 g, 2.79 mmol). The reaction was stirred at room temperature for four hours and then concentrated to remove the organic solvent. The aqueous mixture was acidified (to pH 5-6) with the addition of 1.0 N hydrochloric acid and then extracted with ethyl acetate (15 mL×2). This solution was dried over sodium sulfate and concentrated. The crude product was separated into its component enantiomers by chiral SFC (Thar Prep 80 system; 20×250 mm×10 μm CHIRALCEL IE column; column temperature: 35° C.; column pressure: 100 bar; flow rate: 80 mL/min; eluant: 60:40 methanol/CO₂ with 0.2% ammonia/methanol additive).

Example 43A (first eluting isomer/enantiomer 1) was obtained as a white solid (36.0 mg, 21%). ¹H NMR (400 MHz, CD₃OD) δ 7.73 (s, 1H), 7.67 (d, J=10.8 Hz, 1H), 7.54 (d, J=3.2 Hz, 1H), 7.51-7.48 (m, 1H), 7.27-7.15 (m, 3H), 7.12-7.01 (m, 3H), 4.45-4.39 (m, 1H), 3.32 (s, 3H), 2.96 (t, J=8.0 Hz, 2H), 2.58 (t, J=8.0 Hz, 2H), 1.61 (d, J=6.8 Hz, 3H) ppm. MS: 585 m/z [M+H]⁺.

Example 43B (second eluting isomer/enantiomer 2) was obtained as a white solid (31.3 mg, 19%). ¹H NMR (400 MHz, CD₃OD) δ 7.73 (s, 1H), 7.67 (d, J=10.4 Hz, 1H), 7.54 (d, J=3.2 Hz, 1H), 7.51-7.48 (m, 1H), 7.27-7.15 (m, 3H), 7.12-7.01 (m, 3H), 4.45-4.39 (m, 1H), 3.32 (s, 3H), 2.96 (t, J=8 Hz, 2H), 2.58 (t, J=8.0 Hz, 2H), 1.61 (d, J=7.2 Hz, 3H) ppm. MS: 585 m/z [M+H]⁺.

### Example 44

### 3-(3-(2-(2-(2-Fluoro-5-((6-fluoro-4-(methylsulfonyl)-1H-indol-5-yl)oxy)phenyl)-1H-imidazol-5-yl)-1,3-dioxan-2-yl)phenyl)propanoic acid

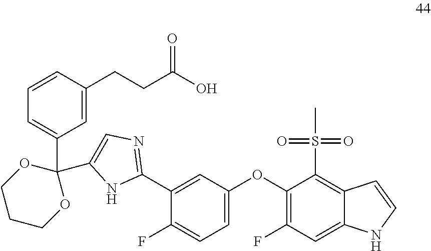 [SMILES: CS(=O)(=O)c1c(Oc2ccc(F)c(-c3ncc(C4(c5cccc(CCC(=O)O)c5)OCCCO4)[nH]3)c2)c(F)cc2[nH]ccc12]

### A. Methyl 3-(3-((2-(2-fluoro-5-((6-fluoro-4-(methylthio)-1H-indol-5-yl)oxy)phenyl)-1H-imidazol-5-yl)(hydroxy)methyl)phenyl)propanoate

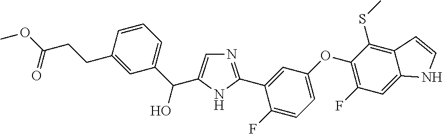 [SMILES: COC(=O)CCc1cccc(C(O)c2cnc(-c3cc(Oc4c(F)cc5[nH]ccc5c4SC)ccc3F)[nH]2)c1]

To a stirred solution of 2-fluoro-5-((6-fluoro-4-(methylthio)-1H-indol-5-yl)oxy)benzimidamide (340 mg, 1.02 mmol, Intermediate 24-4) in DMF (10 mL) was added methyl 3-(3-(3-formyloxiran-2-yl)phenyl)propanoate (0.200 g, 0.851 mmol, Intermediate 26-2). The reaction was heated overnight at 75° C. Following this time, the mixture was cooled and partitioned between water (30 mL) and ethyl acetate (40 mL). The organic layer was combined with additional extracts (ethyl acetate, 2×40 mL), washed with water and brine, dried over sodium sulfate and concentrated. The residue was purified by flash chromatography on silica gel (0-50% ethyl acetate in petroleum ether) to afford the title compound as a colorless oil (0.170 mg, 31%). MS: 550 m/z [M+H]⁺.

### B. Methyl 3-(3-(2-(2-fluoro-5-((6-fluoro-4-(methylthio)-1H-indol-5-yl)oxy)phenyl)-1H-imidazole-5-carbonyl)phenyl)propanoate

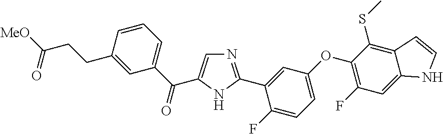 [SMILES: COC(=O)CCc1cccc(C(=O)c2cnc(-c3cc(Oc4c(F)cc5[nH]ccc5c4SC)ccc3F)[nH]2)c1]

To a stirred solution of step A product (0.170 g, 0.309 mmol) in THF (5 mL) was added MnO₂ (0.538 g, 6.19 mmol). The reaction mixture was stirred at room temperature for three hours and then filtered. The captured solids were rinsed with additional THF (2×5 mL) and the combined filtrate was concentrated. The residue was purified by flash chromatography on silica gel (0-50% ethyl acetate in petroleum ether) to afford the title compound as a colorless oil (0.100 g, 59%). MS: 548 m/z [M+H]⁺.

### C. Methyl 3-(3-(2-(2-fluoro-5-((6-fluoro-4-(methylsulfonyl)-1H-indol-5-yl)oxy)phenyl)-1H-imidazole-5-carbonyl)phenyl)propanoate

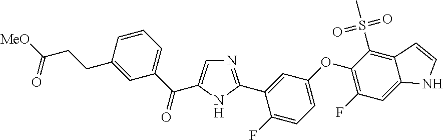 [SMILES: COC(=O)CCc1cccc(C(=O)c2cnc(-c3cc(Oc4c(F)cc5[nH]ccc5c4S(C)(=O)=O)ccc3F)[nH]2)c1]

To a stirred solution of step B product (0.100 g, 0.183 mmol) in methanol (10 mL) was added a solution of ammonium molybdate tetrahydrate (0.200 g, 0.162 mmol) in 1 mL of 30% aqueous hydrogen peroxide. The reaction was stirred at room temperature for two hours and then partitioned between an aqueous sodium sulfite solution (50 mL) and ethyl acetate (35 mL). The organic layer was combined with additional extracts (ethyl acetate, 2×35 mL), washed with an aqueous sodium sulfite solution and brine, dried over sodium sulfate and concentrated. The residue was purified by flash chromatography over silica (0-70% ethyl acetate in petroleum ether) to afford the title compound as a yellow solid (60 mg, 57%). MS: 580 m/z [M+H]⁺.

### D. 3-Hydroxypropyl 3-(3-(2-(2-(2-fluoro-5-((6-fluoro-4-(methylsulfonyl)-1H-indol-5-yl)oxy)phenyl)-1H-imidazol-5-yl)-1,3-dioxan-2-yl)phenyl)propanoate

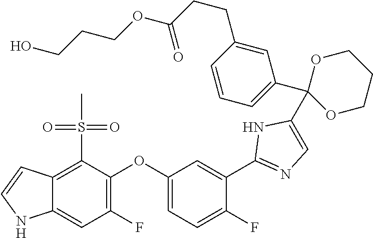 [SMILES: CS(=O)(=O)c1c(Oc2ccc(F)c(-c3ncc(C4(c5cccc(CCC(=O)OCCCO)c5)OCCCO4)[nH]3)c2)c(F)cc2[nH]ccc12]

To a stirred solution of step C product (60.0 mg, 0.104 mmol) in toluene (50 mL) was added p-toluenesulfonic acid (9.0 mg, 47.3 μmol), 1,3-propanediol (500 μL, 0.527 g, 6.92 mmol) and magnesium sulfate (0.240 g, 1.99 mmol). The reaction was heated at reflux for 2 days, cooled to room temperature and filtered. The filtrate was concentrated and the residue was purified by flash chromatography on silica gel (0-50% ethyl acetate in petroleum ether) to afford the title compound as a white solid (20 mg, 28%). MS: 682 m/z [M+H]⁺.

### E. Example 44, 3-(3-(2-(2-(2-Fluoro-5-((6-fluoro-4-(methylsulfonyl)-1H-indol-5-yl)oxy)phenyl)-1H-imidazol-5-yl)-1,3-dioxan-2-yl)phenyl)propanoic acid

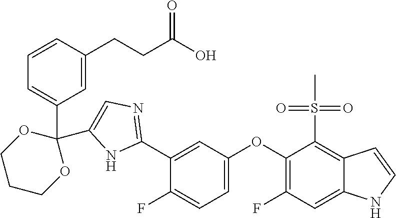 [SMILES: CS(=O)(=O)c1c(Oc2ccc(F)c(-c3ncc(C4(c5cccc(CCC(=O)O)c5)OCCCO4)[nH]3)c2)c(F)cc2[nH]ccc12]

To a stirred solution of step D product (20.0 mg, 29.3 μmol) in a mixture of THF (1 mL) and methanol (0.3 mL) was added a 1.0 M aqueous solution of lithium hydroxide (0.30 mL, 0.30 mmol). After two hours at room temperature, the mixture was acidified (˜pH 4) with 1.0 N hydrochloric acid and then diluted with ethyl acetate (50 mL). This solution was washed with brine (1×50 mL), dried over sodium sulfate and concentrated. The residue was purified by preparative HPLC to afford the title compound as a white solid (1.4 mg, 7%). ¹H NMR (400 MHz, CD₃OD) δ 7.67 (d, J=10.2 Hz, 1H), 7.64-7.59 (m, 1H), 7.53 (d, J=3.2 Hz, 1H), 7.45 (s, 1H), 7.37 (d, J=6.4 Hz, 1H), 7.29 (t, J=7.2 Hz, 1H), 7.23-7.17 (m, 3H), 7.00-6.96 (m, 1H), 6.79 (s, 1H), 4.11-3.96 (m, 4H), 3.33 (s, 3H), 2.93 (t, J=7.6 Hz, 2H), 2.56 (t, J=7.6 Hz, 2H), 1.96-1.86 (m, 1H), 1.74-1.63 (m, 1H) ppm. MS: 624 m/z [M+H]⁺.

### Example 45

### 3-(3-(3-(2-(2-Fluoro-5-((6-fluoro-4-(methylsulfonyl)-1H-indol-5-yl)oxy)phenyl)-1H-imidazol-5-yl)tetrahydrofuran-3-yl)phenyl)propanoic acid

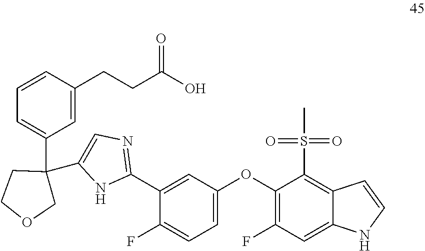 [SMILES: CS(=O)(=O)c1c(Oc2ccc(F)c(-c3ncc(C4(c5cccc(CCC(=O)O)c5)CCOC4)[nH]3)c2)c(F)cc2[nH]ccc12]

### A. Ethyl 3-(3-(3-(2-(2-fluoro-5-((6-fluoro-4-(methylthio)-1H-indol-5-yl)oxy)phenyl)-1H-imidazol-5-yl)tetrahydrofuran-3-yl)phenyl)propanoate

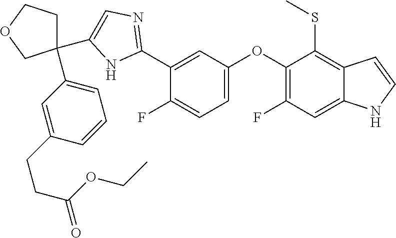 [SMILES: CCOC(=O)CCc1cccc(C2(c3cnc(-c4cc(Oc5c(F)cc6[nH]ccc6c5SC)ccc4F)[nH]3)CCOC2)c1]

To a stirred solution of 2-fluoro-5-((6-fluoro-4-(methylthio)-1H-indol-5-yl)oxy)benzimidamide (0.500 g, 1.36 mmol, Intermediate 24-4) in DMF (10 mL) was added ethyl 3-(3-(3-(2-bromoacetyl)tetrahydrofuran-3-yl)phenyl)propanoate (0.470 g, 1.41 mmol, Intermediate 21-1) and potassium carbonate (0.375 g, 2.72 mmol). The reaction was heated overnight at 75° C. and then diluted with ethyl acetate (60 mL). This solution was washed with brine, dried over sodium sulfate and concentrated. The residue was purified by flash chromatography over silica (40% petroleum ether in 1:1 dichloromethane/ethyl acetate) to afford the title compound as a white solid (0.480 g, 56%). MS: 604 m/z [M+H]⁺.

### B. Ethyl 3-(3-(3-(2-(2-fluoro-5-((6-fluoro-4-(methylsulfonyl)-1H-indol-5-yl)oxy)phenyl)-1H-imidazol-5-yl)tetrahydrofuran-3-yl)phenyl)propanoate

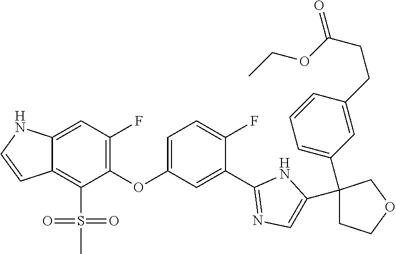 [SMILES: CCOC(=O)CCc1cccc(C2(c3cnc(-c4cc(Oc5c(F)cc6[nH]ccc6c5S(C)(=O)=O)ccc4F)[nH]3)CCOC2)c1]

To a stirred solution of step A product (100 mg, 0.165 mmol) in methanol (10 mL) was added a solution of ammonium molybdate tetrahydrate (0.200 g, 0.162 mmol) in 30% aqueous hydrogen peroxide (1 mL). The reaction mixture was stirred overnight at room temperature and then partitioned between water (30 mL) and ethyl acetate (30 mL). The organic layer was combined with additional extracts (ethyl acetate, 2×15 mL) and washed with an aqueous sodium sulfite solution and brine. The solution was dried over sodium sulfate and concentrated and the residue was purified by flash chromatography over silica (0-60% ethyl acetate in petroleum ether) to afford the title compound as a pale yellow solid (29 mg, 27%). MS: 636 m/z [M+H]⁺.

### C. Example 45, 3-(3-(3-(2-(2-Fluoro-5-((6-fluoro-4-(methylsulfonyl)-1H-indol-5-yl)oxy)phenyl)-1H-imidazol-5-yl)tetrahydrofuran-3-yl)phenyl)propanoic acid

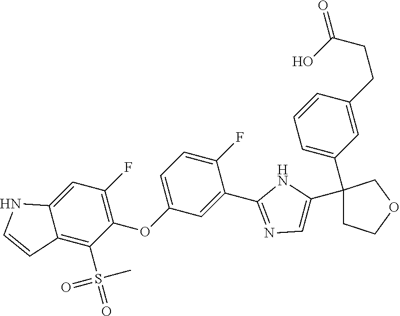 [SMILES: CS(=O)(=O)c1c(Oc2ccc(F)c(-c3ncc(C4(c5cccc(CCC(=O)O)c5)CCOC4)[nH]3)c2)c(F)cc2[nH]ccc12]

To a stirred solution of step B product (50.0 mg, 78.7 μmol) in a mixture of THF (5 mL) and water (1 mL) was added lithium hydroxide monohydrate (15.0 mg, 0.357 mmol). The reaction was continued overnight at 25° C. and then treated, dropwise, with 1.0 N hydrochloric acid until the mixture became acidic (˜pH 2). The resulting precipitate was collected by suction filtration and rinsed with water (3×10 mL). The filtercake was purified by preparative HPLC to afford the title compound as a white solid (15 mg, 31%). ¹H NMR (400 MHz, CD₃OD) δ 7.65 (d, J=10.4 Hz, 1H), 7.55-7.51 (m, 2H), 7.24-7.07 (m, 7H), 6.96-6.89 (m, 1H), 4.39 (d, J=8.4 Hz, 1H), 4.22 (d, J=8.4 Hz, 1H), 4.04-3.94 (m, 2H), 3.33 (s, 3H), 2.88 (t, J=7.6 Hz, 2H), 2.76-2.83 (m, 1H), 2.58-2.48 (m, 3H) ppm. MS: 608 m/z [M+H]⁺.

The following Example compounds were prepared by synthetic routes analogous to that described for Example 45.

| | | | | LCMS | LCMS |
|---|---|---|---|---|---|
| Structure | Ex. No. | Name | ¹H NMR | Rₜ (min) | (m/z) |
| 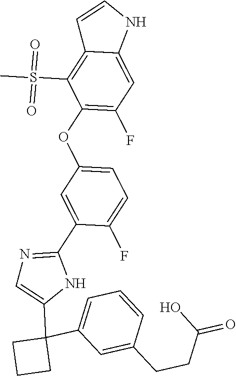 [SMILES: CS(=O)(=O)c1c(Oc2ccc(F)c(-c3ncc(C4(c5cccc(CCC(=O)O)c5)CCC4)[nH]3)c2)c(F)cc2[nH]ccc12] | 46 | 3-(3-(1-(2-(2-fluoro-5-((6- fluoro-4-(methylsulfonyl)- 1H-indol-5-yl)oxy)phenyl)- 1H-imidazol-5- yl)cyclobutyl)phenyl) propanoic acid | (400 MHz, CD₃OD) δ 7.67 (d, J = 10.4 Hz, 1H), 7.54 (d, J = 3.2 Hz, 1H), 7.48 (dd, J = 5.6, 3.2 Hz, 1H), 7.30-7.17 (m, 6H), 7.11-7.05 (m, 2H), 3.34 (s, 3H), 2.91 (t, J = 7.6 Hz, 2H), 2.73 (t, J = 7.6 Hz, 4H), 2.59 (t, J = 7.6 Hz, 2H), 2.17-1.96 (m, 2H) ppm. | 0.87 | 592 [M + H]⁺ |
| 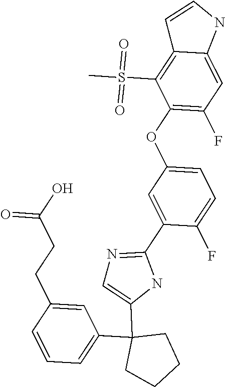 | 47 | 3-(3-(1-(2-(2-fluoro-5-((6- fluoro-4-(methylsulfonyl)- 1H-indol-5-yl)oxy)phenyl)- 1H-imidazol-5- yl)cyclopentyl)phenyl) propanoic acid | (400 MHz, CD3OD) δ 7.65 (d, J = 10.4 Hz, 1H), 7.52 (d, J = 2.8 Hz, 1H), 7.49-7.47 (m, 1H), 7.23 (s, 1H), 7.20-7.15 (m, 4H), 7.03 (d, J = 6.0 Hz, 1H), 7.00 (s, 1H), 6.93-6.90 (m, 1H ), 3.33 (s, 3H), 2.88 (t, J = 7.6 Hz, 2H), 2.54 (t, J = 7.6 Hz, 2H), 2.46-2.41 (m, 2H), 2.22-2.19 (m, 2H), 1.78-1.77 (m, 4H) ppm. | 0.90 | 606 [M + H]⁺ |

### Example 48

### 3-(3-(1-Cyano-1-(2-(2-fluoro-5-((6-fluoro-4-(methylsulfonyl)-1H-indol-5-yl)oxy)phenyl)-1H-imidazol-4-yl)ethyl)phenyl)propanoic acid

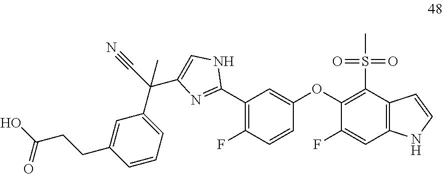 [SMILES: CC(C#N)(c1cccc(CCC(=O)O)c1)c1c[nH]c(-c2cc(Oc3c(F)cc4[nH]ccc4c3S(C)(=O)=O)ccc2F)n1]

### A. 2-(3-Iodophenyl)propanoic acid

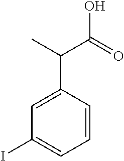

To a stirred and cooled (−78° C.) solution of 2-(3-iodophenyl)acetic acid (6.00 g, 22.9 mmol) in THF (100 mL) was added, dropwise, a 2.0 M solution of LDA in a mixture of THF, heptane and ethylbenzene (34.2 mL, 68.4 mmol). The mixture was stirred at −78° C. for one hour and then treated, dropwise, with iodomethane (4.56 mL, 73.2 mmol). Following the addition, the cooling bath was removed and the reaction was allowed to slowly warm to room temperature. Stirring was continued overnight before the reaction was quenched with the addition of water (˜50 mL). The mixture was then made acidic (˜pH 2) by the addition of 2N hydrochloric acid and extracted with ethyl acetate (3×70 mL). The combined extracts were washed with brine, dried over sodium sulfate and concentrated. The residue was purified by flash chromatography over silica (0-45% ethyl acetate in petroleum ether) to afford the title compound as an oil (5.50 g, 87%). ¹H NMR (400 MHz, DMSO-d₆) δ 12.44 (br s, 1H), 7.66-7.61 (m, 2H), 7.32 (d, J=8.0 Hz, 1H), 7.14 (t, J=8.0 Hz, 1H), 3.68 (q, J=7.2 Hz, 1H), 1.35 (d, J=7.2 Hz, 3H) ppm.

### B. 3-Hydroxy-2-(3-iodophenyl)-2-methylpropanoic acid

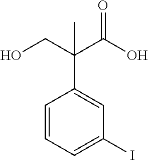

To a stirred and cooled (−78° C.) solution of step A product (4.50 g, 16.3 mmol) in THF (50 mL) was added, dropwise, a 2.0 M solution of LDA in a mixture of THF, heptane and ethylbenzene (24.4 mL, 48.8 mmol). The mixture was stirred at −78° C. for one hour and then treated with paraformaldehyde (0.978 g, 32.6 mmol). Following the addition, the cooling bath was removed and the reaction was allowed to slowly warm to room temperature. Stirring was continued overnight before the reaction was quenched with the addition of water (˜50 mL). The mixture was then made acidic (˜pH 2) by the addition of 2N hydrochloric acid and extracted with ethyl acetate (3×70 mL). The combined extracts were washed with brine, dried over sodium sulfate and concentrated. The residue was purified by flash chromatography over silica (0-50% ethyl acetate in petroleum ether) to afford the title compound as an oil (4.00 g, 80%). ¹H NMR (400 MHz, DMSO-d₆) δ 12.47 (br s, 1H), 7.63-7.61 (m, 2H), 7.35 (d, J=8.0 Hz, 1H), 7.14 (t, J=7.6 Hz, 1H), 4.95 (br s, 1H), 3.86 (d, J=10.0 Hz, 1H), 3.67 (d, J=10.4 Hz, 1H), 1.45 (s, 3H) ppm.

### C. 3-Acetoxy-2-(3-iodophenyl)-2-methylpropanoic acid

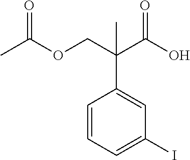 [SMILES: CC(=O)OCC(C)(C(=O)O)c1cccc(I)c1]

To a stirred solution of step B product (4.35 g, 14.2 mmol) in dichloromethane (100 mL) was added triethylamine (7.97 mL, 56.8 mmol), acetic anhydride (2.00 mL, 21.2 mmol) and DMAP (86.0 mg, 0.704 mmol). The reaction was stirred at room temperature for two hours and then quenched with the addition of water (˜100 mL). After concentrating to remove the bulk of the halogenated solvent, the mixture was extracted with ethyl acetate (3×75 mL). The combined organic extracts were washed with brine, dried over sodium sulfate and concentrated. The residue was purified by flash chromatography over silica (0-50% ethyl acetate in petroleum ether) to afford the title compound as an oil (3.80 g, 77%). MS: 371 m/z [M+Na]⁺.

### D. 4-Bromo-2-(3-iodophenyl)-2-methyl-3-oxobutyl acetate

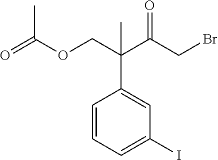 [SMILES: CC(=O)OCC(C)(C(=O)CBr)c1cccc(I)c1]

To a stirred solution of step C product (3.80 g, 10.9 mmol) in dichloromethane (40 mL) was added oxalyl chloride (1.66 mL, 19.6 mmol) followed by one drop of DMF. The reaction mixture was stirred at room temperature for three hours and then concentrated to afford the crude acid chloride as an oil (3.99 g, 100%). The unpurified intermediate was taken up in a mixture of THF (25 mL) and acetonitrile (25 mL). To this stirred and cooled (0° C.) solution was added, dropwise, a 2.0 M solution of trimethylsilyldiazomethane in hexanes (21.8 mL, 43.6 mmol). Following the addition, the cooling bath was removed and the reaction mixture was allowed to slowly warm to room temperature. Stirring was continued overnight before the mixture was returned to 0° C. and treated, dropwise over 10 minutes, with a 32% solution of hydrogen bromide in acetic acid (9.38 mL, 43.6 mmol). The reaction was allowed to gradually warm to room temperature, stirred for another 30 minutes and then diluted with brine (100 mL). The biphasic mixture was extracted with ethyl acetate (3×50 mL). The combined extracts were then washed with brine, dried over magnesium sulfate and concentrated. The residue was purified by flash chromatography over silica (0-30% ethyl acetate in petroleum ether) to afford the title compound as an oil (3.49 g, 75%). ¹H NMR (400 MHz, CDCl₃) δ 7.70-7.59 (m, 2H), 7.20 (d, J=8.0 Hz, 1H), 7.14 (t, J=8.0 Hz, 1H), 4.56 (d, J=11.2 Hz, 1H), 4.34 (d, J=11.2 Hz, 1H), 3.96 (d, J=14.0 Hz, 1H), 3.87 (d, J=14.0 Hz, 1H), 2.03 (s, 3H), 1.66 (s, 3H) ppm.

### E. 2-(2-(2-Fluoro-5-((6-fluoro-4-(methylthio)-1H-indol-5-yl)oxy)phenyl)-1H-imidazol-5-yl)-2-(3-iodophenyl)propyl acetate

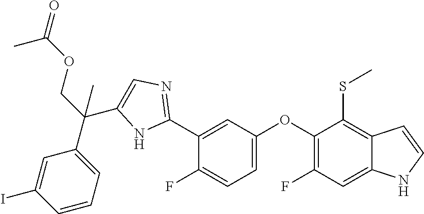 [SMILES: CSc1c(Oc2ccc(F)c(-c3ncc(C(C)(COC(C)=O)c4cccc(I)c4)[nH]3)c2)c(F)cc2[nH]ccc12]

To a stirred solution of 2-fluoro-5-((6-fluoro-4-(methylthio)-1H-indol-5-yl)oxy)benzimidamide (2.40 g, 7.21 mmol, Intermediate 24-4) in DMF (40 mL) was added step D product (3.10 g, 7.21 mmol) and potassium carbonate (2.00 g, 14.5 mmol). The reaction was heated overnight at 75° C. and then cooled to room temperature and diluted with ethyl acetate (200 mL). This solution was washed with water (4×50 mL) and brine (2×50 mL), dried over sodium sulfate and concentrated. The residue was purified by flash chromatography over silica (45% petroleum ether in 1:1 ethyl acetate/dichloromethane) to afford the title compound as a light yellow solid (3.30 g, 70%). MS: 660 m/z [M+H]⁺.

### F. 2-(2-(2-Fluoro-5-((6-fluoro-4-(methylthio)-1H-indol-5-yl)oxy)phenyl)-1H-imidazol-5-yl)-2-(3-iodophenyl)propan-1-ol

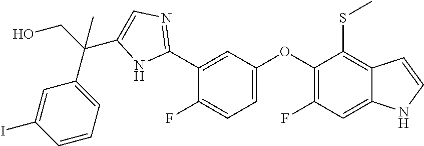 [SMILES: CSc1c(Oc2ccc(F)c(-c3ncc(C(C)(CO)c4cccc(I)c4)[nH]3)c2)c(F)cc2[nH]ccc12]

To a stirred solution of step E product (0.500 g, 0.758 mmol) in a mixture of THF (10 mL) and methanol (1 mL) was added a solution of lithium hydroxide (0.073 g, 3.05 mmol) in water (1.0 mL). The reaction was stirred at room temperature for two hours and then diluted with ethyl acetate (100 mL). The solution was washed with water (2×30 mL) and brine (1×30 mL), dried over sodium sulfate and concentrated. The residue was purified by flash chromatography over silica (50% dichloromethane in 1:1 ethyl acetate/petroleum ether) to afford the title compound as a white solid (0.400 g, 85%). MS: 618 m/z [M+H]⁺.

### G. 2-(2-(2-Fluoro-5-((6-fluoro-4-(methylthio)-1H-indol-5-yl)oxy)phenyl)-1H-imidazol-5-yl)-2-(3-iodophenyl)propanal

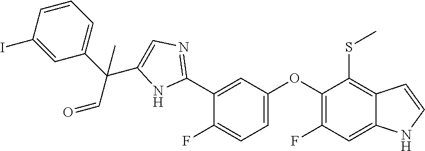 [SMILES: CSc1c(Oc2ccc(F)c(-c3ncc(C(C)(C=O)c4cccc(I)c4)[nH]3)c2)c(F)cc2[nH]ccc12]

To a stirred solution of step F product (0.400 g, 0.648 mmol) in DMSO (15 mL) was added Dess-Martin periodinane (0.461 g, 1.09 mmol). The reaction was stirred at room temperature for one hour and then diluted with saturated, aqueous sodium thiosulfate solution (20 mL). The mixture was stirred for another 20 minutes and extracted with ethyl acetate (1×100 mL). The organic phase was then washed with aqueous sodium bicarbonate solution (1×30 mL), water (1×30 mL) and brine (1×30 mL), dried with sodium sulfate and concentrated. The residue was purified by flash chromatography over silica (33% ethyl acetate in petroleum ether) to afford the title compound as a white solid (0.287 g, 72%). MS: 616 m/z [M+H]⁺.

### H. 2-(2-(2-Fluoro-5-((6-fluoro-4-(methylthio)-1H-indol-5-yl)oxy)phenyl)-1H-imidazol-5-yl)-2-(3-iodophenyl)propanenitrile

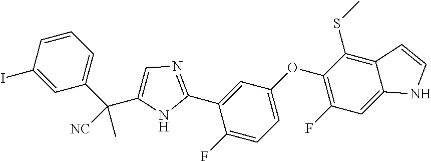 [SMILES: CSc1c(Oc2ccc(F)c(-c3ncc(C(C)(C#N)c4cccc(I)c4)[nH]3)c2)c(F)cc2[nH]ccc12]

To a stirred solution of step G product (0.287 g, 0.466 mmol) in DMF (4 mL) was added a 50 wt % solution of propanephosphonic acid cyclic anhydride in DMF (1.36 mL, 2.33 mmol), hydroxylamine hydrochloride (0.161 g, 2.32 mmol) and triethylamine (0.52 mL, 3.73 mmol). The reaction vessel was sealed and heated overnight at 125° C. The mixture was cooled to room temperature and diluted with ethyl acetate (100 mL). This solution was washed with water (4×40 mL) and brine (2×40 mL), dried over sodium sulfate and concentrated. The residue was purified by flash chromatography over silica (33% ethyl acetate in petroleum ether) to afford the title compound as a white solid (0.170 g, 59%). MS: 613 m/z [M+H]⁺.

### I. Ethyl (E)-3-(3-(1-cyano-1-(2-(2-fluoro-5-((6-fluoro-4-(methylthio)-1H-indol-5-yl)oxy)phenyl)-1H-imidazol-5-yl)ethyl)phenyl)acrylate

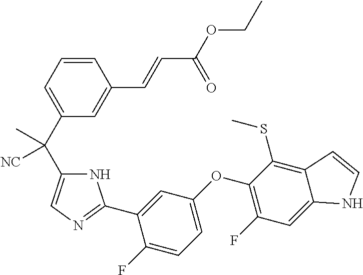 [SMILES: CCOC(=O)/C=C/c1cccc(C(C)(C#N)c2cnc(-c3cc(Oc4c(F)cc5[nH]ccc5c4SC)ccc3F)[nH]2)c1]

To a stirred solution of step H product (0.170 g, 0.278 mmol) in DMF (7 mL) was added ethyl acrylate (90.4 μL, 0.829 mmol), palladium(II) acetate (7.0 mg, 31 μmol), tri(o-tolyl)phosphine (26.0 mg, 85.4 μmol) and triethylamine (117 μL, 0.839 mmol). The reaction vessel was sealed and heated at 100° C. for three hours. After this time, the mixture was cooled and concentrated. The residue was purified by flash chromatography over silica (0-10% methanol in dichloromethane) to afford the title compound as a light yellow solid (0.135 g, 83%). MS: 585 m/z [M+H]⁺.

### J. Ethyl 3-(3-(1-cyano-1-(2-(2-fluoro-5-((6-fluoro-4-(methylthio)-1H-indol-5-yl)oxy)phenyl)-1H-imidazol-5-yl)ethyl)phenyl)propanoate

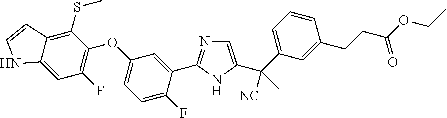 [SMILES: CCOC(=O)CCc1cccc(C(C)(C#N)c2cnc(-c3cc(Oc4c(F)cc5[nH]ccc5c4SC)ccc3F)[nH]2)c1]

A stirred solution of step I product (0.135 g, 0.231 mmol) and 10% Pd/C (0.060 g, 56.4 μmol) in THF (10 mL) was cycled between vacuum and a nitrogen atmosphere three times. The suspension was evacuated a final time and refilled with hydrogen (via balloon). The reaction was stirred overnight at room temperature and then suction filtered through a pad of Celite. The filtering agent was rinsed with additional THF (3×10 mL) and the combined filtrate was concentrated to afford the crude title compound as a light yellow solid (0.130 g, 96%). MS: 587 m/z [M+H]⁺.

### K. Ethyl 3-(3-(1-cyano-1-(2-(2-fluoro-5-((6-fluoro-4-(methylsulfonyl)-1H-indol-5-yl)oxy)phenyl)-1H-imidazol-5-yl)ethyl)phenyl)propanoate

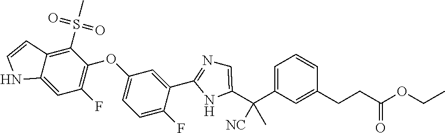 [SMILES: CCOC(=O)CCc1cccc(C(C)(C#N)c2cnc(-c3cc(Oc4c(F)cc5[nH]ccc5c4S(C)(=O)=O)ccc3F)[nH]2)c1]

To a stirred solution of step J product (0.130 g, 0.222 mmol) in methanol (10 mL) was added a solution of ammonium molybdate tetrahydrate (0.200 g, 0.162 mmol) in 30% aqueous hydrogen peroxide (2 mL). The reaction was stirred at room temperature for two hours and then diluted with ethyl acetate (40 mL). The solution was washed with aqueous sodium sulfite solution (2×30 mL) and brine (1×20 mL), dried over sodium sulfate and concentrated. The residue was purified by flash chromatography over silica (5% ethyl acetate in petroleum ether) to afford the title as a white solid (58.0 mg, 42%). MS: 619 m/z [M+H]⁺.

### L. Example 48, 3-(3-(1-Cyano-1-(2-(2-fluoro-5-((6-fluoro-4-(methylsulfonyl)-1H-indol-5-yl)oxy)phenyl)-1H-imidazol-5-yl)ethyl)phenyl)propanoic acid

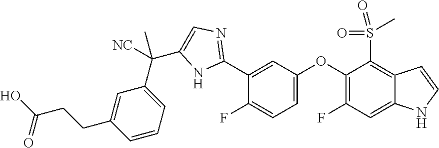 [SMILES: CC(C#N)(c1cccc(CCC(=O)O)c1)c1cnc(-c2cc(Oc3c(F)cc4[nH]ccc4c3S(C)(=O)=O)ccc2F)[nH]1]

To a stirred solution of step K product (58.0 mg, 93.8 μmol) in THF (8 mL) was added a solution of lithium hydroxide (11.0 mg, 0.459 mmol) in water (0.5 mL). The reaction was stirred overnight at room temperature and then diluted with water (10 mL) and made acidic (˜pH 6.5) with the addition of 1.0 N hydrochloric acid. The resulting suspension was suction filtered and the collected solid was rinsed with additional water (2×1 mL) and vacuum oven dried to afford the title compound as a white solid (40.5 mg, 70%). ¹H NMR (400 MHz, CD₃OD) δ 7.66-7.61 (m, 2H), 7.52 (d, J=3.2 Hz, 1H), 7.37 (s, 1H), 7.33-7.30 (m, 2H), 7.23-7.18 (m, 3H), 7.10 (s, 1H), 6.95-6.92 (m, 1H), 3.33 (s, 3H), 2.93 (t, J=7.6 Hz, 2H), 2.59 (t, J=7.6 Hz, 2H), 2.08 (s, 3H) ppm. MS: 591 m/z [M+H]⁺.

### Example 49

### 3-(3-(1-Cyano-1-(2-(5-((6,7-difluoro-4-(methylsulfonyl)-1H-indol-5-yl)oxy)-2-fluorophenyl)-1H-imidazol-5-yl)ethyl)phenyl)propanoic acid

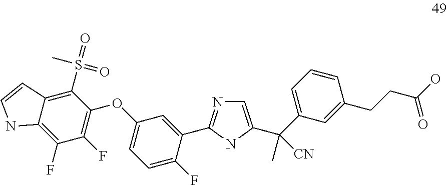 [SMILES: CC(C#N)(c1cccc(CCC(=O)O)c1)c1cnc(-c2cc(Oc3c(F)c(F)c4[nH]ccc4c3S(C)(=O)=O)ccc2F)[nH]1]

Exchanging 2-fluoro-5-((6-fluoro-4-(methylthio)-1H-indol-5-yl)oxy)benzimidamide (Intermediate 24-4) for 5-((6,7-difluoro-4-(methylthio)-1H-indol-5-yl)oxy)-2-fluorobenzimidamide (Intermediate 24-8) in Step E, the 12-step reaction sequence (Steps A to L) described for Example 48 was used to prepare the title compound as a white solid. ¹H NMR (400 MHz, CD₃OD) δ 7.64 (dd, J=5.6, 3.2 Hz, 1H), 7.58 (d, J=3.2 Hz, 1H), 7.37 (s, 1H), 7.32-7.31 (m, 2H), 7.26-7.21 (m, 3H), 7.11 (s, 1H), 7.00 (dt, J=9.2, 3.2 Hz, 1H), 3.32 (s, 3H), 2.93 (t, J=7.6 Hz, 2H), 2.60 (t, J=7.6 Hz, 2H), 2.08 (s, 3H) ppm. MS: 609 m/z [M+H]⁺.

### Example 50

### 3-(3-(1-cyano-1-(2-(2-fluoro-5-((6-fluoro-4-methyl-1H-indol-5-yl)oxy)phenyl)-1H-imidazol-5-yl)ethyl)phenyl)propanoic acid

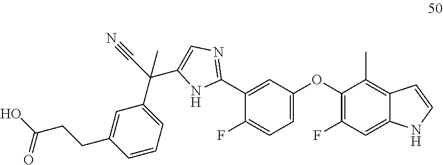 [SMILES: Cc1c(Oc2ccc(F)c(-c3ncc(C(C)(C#N)c4cccc(CCC(=O)O)c4)[nH]3)c2)c(F)cc2[nH]ccc12]

The title compound was prepared by adapting the 12-step synthesis described for Example 48. The following changes were made to the route: iodomethane was exchanged for iodomethane-d₃ in step A, 2-fluoro-5-((6-fluoro-4-(methylthio)-1H-indol-5-yl)oxy)benzimidamide (Intermediate 24-4) was exchanged for 2-fluoro-5-((6-fluoro-4-methyl-1H-indol-5-yl)oxy)benzimidamide (Intermediate 24-10) in step E and the methylthio to methylsulfonyl oxidation (step K) was omitted. This modified, 11-step synthesis was used to prepare the title compound as a white solid. ¹H NMR (400 MHz, CD₃OD) δ 7.47 (dd, J=6.0, 3.2 Hz, 1H), 7.36 (s, 1H), 7.32-7.31 (m, 2H), 7.27 (d, J=3.2 Hz, 1H), 7.23-7.22 (m, 1H), 7.18-7.10 (m, 2H), 7.07 (s, 1H), 6.83 (dt, J=9.2, 3.6 Hz, 1H), 6.52 (d, J=3.2 Hz, 1H), 2.92 (t, J=7.6 Hz, 2H), 2.58 (t, J=7.6 Hz, 2H), 2.39 (s, 3H), 2.08 (s, 3H) ppm. MS: 527 m/z [M+H]⁺.

The following Example compounds were prepared by synthetic routes analogous to that described for Example 50.

| | | | | LCMS | MS |
|---|---|---|---|---|---|
| Structure | Ex. No. | Name | ¹H NMR | Rₜ | m/z |
| 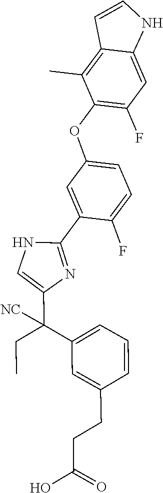 [SMILES: CCC(C#N)(c1cccc(CCC(=O)O)c1)c1c[nH]c(-c2cc(Oc3c(F)cc4[nH]ccc4c3C)ccc2F)n1] | 51 | 3-(3-(1-cyano-1-(2-(2-fluoro- 5-((6-fluoro-4-methyl-1H- indol-5-yl)oxy)phenyl)-1H- imidazol-4- yl)propyl)phenyl)propanoic acid | (400 MHz, CD₃OD) δ 7.47-7.45 (m, 1H), 7.37 (s, 1H), 7.33-7.26 (m, 3H), 7.20-7.07 (m, 4H), 6.82-6.79 (m, 1H), 6.50 (d, J = 2.8 Hz, 1H), 2.90 (t, J = 7.6 Hz, 2H), 2.60-2.55 (m, 3H), 2.37 (s, 3H), 2.34-2.30 (m, 1H), 0.98 (t, J = 7.6 Hz, 3H) ppm. | 1.34 | 541 |
| 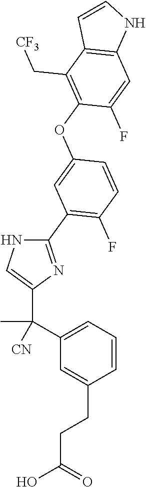 [SMILES: CC(C#N)(c1cccc(CCC(=O)O)c1)c1c[nH]c(-c2cc(Oc3c(F)cc4[nH]ccc4c3CC(F)(F)F)ccc2F)n1] | 52 | 3-(3-(1-cyano-1-(2-(2-fluoro- 5-((6-fluoro-4-(2,2,2- trifluoroethyl)-1H-indol-5- yl)oxy)phenyl)-1H-imidazol- 4-yl)ethyl)phenyl)propanoic acid | (400 MHz, CD₃OD) δ 7.49 (dd, J = 3.2, 5.6 Hz, 1H), 7.35 (d, J = 2.4 Hz, 2H), 7.31-7.28 (m, 3H), 7.22-7.16 (m, 2H), 7.09 (s, 1H), 6.90-6.86 (m, 1H), 6.58 (d, J = 2.8 Hz, 1H), 3.75 (q, J = 10.8 Hz, 2H), 2.92 (t, J = 7.6 Hz, 2H), 2.59 (t, J = 7.6 Hz, 2H), 2.08 (s, 3H) ppm. | 1.31 | 595 |
| 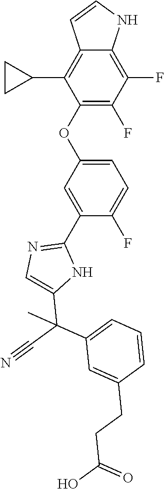 [SMILES: CC(C#N)(c1cccc(CCC(=O)O)c1)c1cnc(-c2cc(Oc3c(F)c(F)c4[nH]ccc4c3C3CC3)ccc2F)[nH]1] | 53 | 3-(3-(1-cyano-1-(2-(5-((4- cyclopropyl-6,7-difluoro-1H- indol-5-yl)oxy)-2- fluorophenyl)-1H-imidazol-5- yl)ethyl)phenyl)propanoic acid | (400 MHz, CD₃OD) δ 7.50 (s, 1H), 7.44-7.29 (m, 7H), 7.17- 7.12 (m, 1H), 6.68 (s, 1H), 2.95 (t, J = 7.2 Hz, 2H), 2.61 (d, J = 7.2 Hz, 2H), 2.16 (s, 3H), 1.95- 1.90 (m, 1H), 0.92-0.88 (m, 4H) ppm. | 1.37 | 571 |
| 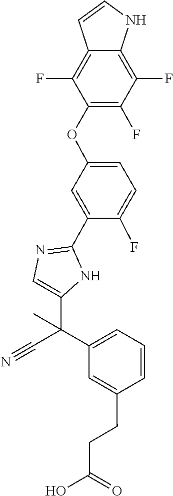 [SMILES: CC(C#N)(c1cccc(CCC(=O)O)c1)c1cnc(-c2cc(Oc3c(F)c(F)c4[nH]ccc4c3F)ccc2F)[nH]1] | 54 | 3-(3-(1-cyano-1-(2-(2-fluoro- 5-((4,6,7-trifluoro-1H-indol- 5-yl)oxy)phenyl)-1H- imidazol-5- yl)ethyl)phenyl)propanoic acid | (400 MHz, CD₃OD) δ 7.57 (dd, J = 6.0, 3.2 Hz, 1H), 7.38 (d, J = 3.6 Hz, 1H), 7.37 (s, 1H), 7.32-7.30 (m, 2H), 7.27-7.21 (m, 2H), 7.10 (s, 1H), 7.04-6.99 (m, 1H), 6.64 (t, J = 2.8 Hz, 1H), 2.93 (t, J = 7.6 Hz, 2H), 2.60 (t, J = 7.6 Hz, 2H), 2.08 (s, 3H) ppm. | 1.28 | 549 |
| 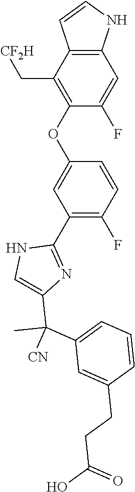 [SMILES: CC(C#N)(c1cccc(CCC(=O)O)c1)c1c[nH]c(-c2cc(Oc3c(F)cc4[nH]ccc4c3CC(F)F)ccc2F)n1] | 55 | 3-(3-(1-cyano-1-(2-(5-((4- (2,2-difluoroethyl)-6-fluoro- 1H-indol-5-yl)oxy)-2- fluorophenyl)-1H-imidazol-4- yl)ethyl)phenyl)propanoic acid | (400 MHz, CD₃OD) δ 7.51-7.48 (m, 1H), 7.41-7.32 (m, 4H), 7.27-7.19 (m, 4H), 6.94-6.91 (m, 1H), 6.59 (d, J = 3.6 Hz, 1H), 6.11 (tt, J = 5.2, 56.8 Hz, , 1H), 3.46-3.38 (m, 2H), 2.93 (t, J = 7.6 Hz, 2H), 2.60 (t, J = 7.6 Hz, 2H), 2.09 (s, 3H) ppm. | 1.26 | 577 |
| 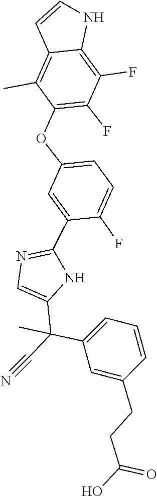 [SMILES: Cc1c(Oc2ccc(F)c(-c3ncc(C(C)(C#N)c4cccc(CCC(=O)O)c4)[nH]3)c2)c(F)c(F)c2[nH]ccc12] | 56 | 3-(3-(1-cyano-1-(2-(5-((6,7- difluoro-4-methyl-1H-indol- 5-yl)oxy)-2-fluorophenyl)- 1H-imidazol-5- yl)ethyl)phenyl)propanoic acid | (400 MHz, CD₃OD) δ 7.48 (dd, J = 10.0, 3.2 Hz, 1H), 7.38-7.34 (m, 2H), 7.30 (d, J = 4.8 Hz, 2H), 7.26-7.15 (m, 2H), 7.07 (s, 1H), 6.86 (dt, J = 8.8, 3.6 Hz, 1H), 6.58 (t, J = 3.2 Hz, 1H), 2.92 (t, J = 7.6 Hz, 2H), 2.55 (t, J = 7.6 Hz, 2H), 2.34 (s, 3H), 2.08 (s, 3H) ppm. | 1.31 | 545 |
| 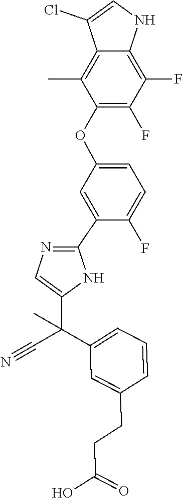 [SMILES: Cc1c(Oc2ccc(F)c(-c3ncc(C(C)(C#N)c4cccc(CCC(=O)O)c4)[nH]3)c2)c(F)c(F)c2[nH]cc(Cl)c12] | 57 | 3-(3-(1-(2-(5-((3-chloro-6,7- difluoro-4-methyl-1H-indol- 5-yl)oxy)-2-fluorophenyl)- 1H-imidazol-5-yl)-1- cyanoethyl)phenyl)propanoic acid | (400 MHz, CD₃OD) δ 7.47 (dd, J = 6.0, 3.2 Hz, 1H), 7.37-29 (m, 4H), 7.23-7.18 (m, 2H), 7.07 (s, 1H), 6.89 (dt, J = 9.6, 3.6 Hz, 1H), 2.92 (t, J = 7.6 Hz, 2H), 2.59 (s, 3H), 2.53 (t, J = 7.6 Hz, 2H), 2.08 (s, 3H) ppm. | 1.41 | 579 |
| 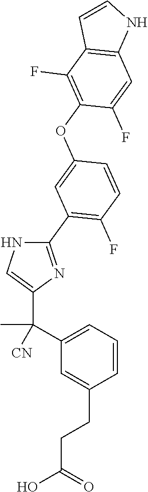 [SMILES: CC(C#N)(c1cccc(CCC(=O)O)c1)c1c[nH]c(-c2cc(Oc3c(F)cc4[nH]ccc4c3F)ccc2F)n1] | 58 | 3-(3-(1-cyano-1-(2-(5-((4,6- difluoro-1H-indol-5-yl)oxy)- 2-fluorophenyl)-1H-imidazol- 4-yl)ethyl)phenyl)propanoic acid | (400 MHz, CD₃OD) δ 7.55 (dd, J = 6.4, 3.2 Hz, 1H), 7.37 (s, 1H), 7.31 (d, J = 3.6Hz, 3H), 7.22-7.18 (m, 2H), 7.15 (d, J = 10.0 Hz, 1H), 7.07 (s, 1H), 7.00-6.94 (m, 1H), 6.56 (d, J = 2.8 Hz, 1H), 2.93 (t, J = 7.2 Hz, 2H), 2.59 (t, J = 7.2 Hz, 2H), 2.08 (s, 3H ) ppm. | 1.31 | 531 |
| 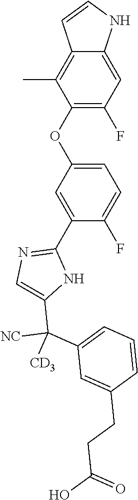 [SMILES: [2H]C([2H])([2H])C(C#N)(c1cccc(CCC(=O)O)c1)c1cnc(-c2cc(Oc3c(F)cc4[nH]ccc4c3C)ccc2F)[nH]1] | 59 | 3-(3-(1-cyano-1-(2-(2-fluoro- 5-((6-fluoro-4-methyl-1H- indol-5-yl)oxy)phenyl)-1H- imidazol-5-yl)ethyl-2,2,2- d₃)phenyl)propanoic acid | (400 MHz, CD₃OD) δ 7.47 (dd, J = 6.0, 3.2 Hz, 1H), 7.37 (s, 1H), 7.31 (d, J = 5.2 Hz, 2H), 7.27 (d, J = 2.8 Hz, 1H), 7.23- 7.10 (m, 3H), 7.08 (s, 1H), 6.84 (dt, J = 8.8, 3.6 Hz, 1H), 6.52 (d, J = 3.2 Hz, 1H), 2.93 (t, J = 7.6 Hz, 2H), 2.60 (t, J = 7.6 Hz, 2H), 2.39 (s, 3H) ppm. | 1.25 | 530 |
| 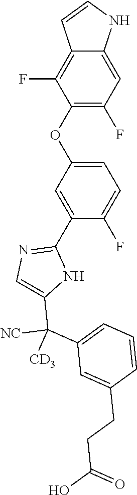 [SMILES: [2H]C([2H])([2H])C(C#N)(c1cccc(CCC(=O)O)c1)c1cnc(-c2cc(Oc3c(F)cc4[nH]ccc4c3F)ccc2F)[nH]1] | 60 | 3-(3-(1-cyano-1-(2-(5-((4,6- difluoro-1H-indol-5-yl)oxy)- 2-fluorophenyl)-1H-imidazol- 5-yl)ethyl-2,2,2- d₃)phenyl)propanoic acid | (400 MHz, CD₃OD) δ 7.55 (dd, J = 5.6, 2.8 Hz, 1H), 7.37 (s, 1H), 7.31-7.30 (m, 3H), 7.22- 7.12 (m, 3H), 7.07 (s, 1H), 6.96 (dt, J = 9.2, 3.2 Hz, 1H),, 6.56 (d, J = 3.2 Hz, 1H), 2.92 (t, J = 7.6 Hz, 2H), 2.59 (t, J = 7.6 Hz, 2H) ppm. | 1.24 | 534 |
| 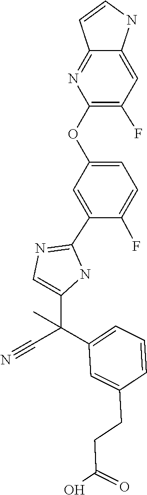 | 61 | 3-(3-(1-cyano-1-(2-(2-fluoro- 5-((6-fluoro-1H-pyrrolo[3,2- b]pyridin-5-yl)oxy)phenyl)- 1H-imidazol-5- yl)ethyl)phenyl)propanoic acid | (400 MHz, CD₃OD) δ 7.77 (dd, J = 10.4, 0.8 Hz, 1H), 7.73 (dd, J = 6.0, 2.8Hz, 1H), 7.51 (d, J = 3.2 Hz, 1H), 7.37 (s, 1H), 7.32- 7.27 (m, 3H), 7.24-7.13 (m, 2H), 7.11 (s, 1H), 6.44 (dd, J = 3.2, 0.8 Hz, 1H), 2.92 (t, J = 7.6 Hz, 2H), 2.59 (t, J = 7.6 Hz, 2H), 2.08 (s, 3H) ppm. | 1.06 | 514 |
| 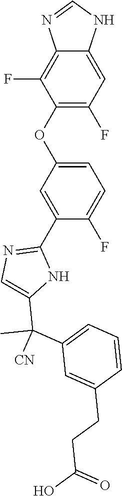 [SMILES: CC(C#N)(c1cccc(CCC(=O)O)c1)c1cnc(-c2cc(Oc3c(F)cc4[nH]cnc4c3F)ccc2F)[nH]1] | 62 | 3-(3-(1-cyano-1-(2-(5-((4,6- difluoro-1H- benzo[d]imidazol-5-yl)oxy)- 2-fluorophenyl)-1H-imidazol- 5-yl)ethyl)phenyl)propanoic acid | (400 MHz, CD3OD) δ 8.28 (s, 1H), 7.57 (d, J = 3.2 Hz, 1H), 7.33-7.41 (m, 2H), 7.17-7.33 (m, 4H), 7.08 (s, 1H), 7.00-7.06 (m, 1H), 2.91 (t, J = 7.6 Hz, 2H), 2.53 (t, J = 7.6 Hz, 2H), 2.07 (s, 3H) ppm. | 0.98 | 532 |
| 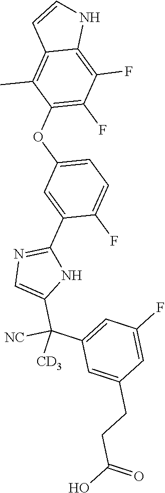 [SMILES: [2H]C([2H])([2H])C(C#N)(c1cc(F)cc(CCC(=O)O)c1)c1cnc(-c2cc(Oc3c(F)c(F)c4[nH]ccc4c3C)ccc2F)[nH]1] | 63 | 3-(3-(1-cyano-1-(2-(5-((6,7- difluoro-4-methyl-1H-indol- 5-yl)oxy)-2-fluorophenyl)- 1H-imidazol-5-yl)ethyl-2,2,2- d3)-5-fluorophenyl)propanoic acid | (400 MHz, CD₃OD) δ 7.49 (dd, J = 2.8, 6.0 Hz, 1H), 7.35 (d, J = 7.6 Hz, 1H), 7.21-7.15 (m, 3H), 7.04-6.98 (m, 2H), 6.89- 6.85 (m, 2H), 6.59 (t, J = 3.6 Hz, 1H), 2.93 (t, J = 7.6 Hz, 2H), 2.59 (t, J = 7.6 Hz, 2H), 2.35 (s, 3H) ppm | 1.33 | 566 |

### Example 64

### Enantiomer 1 of 3-(3-(1-Cyano-1-(2-(2-fluoro-5-((4,6,7-trifluoro-1H-indol-5-yl)oxy)phenyl)-1H-imidazol-5-yl)ethyl-2,2,2-d3)phenyl)propanoic acid (64A)

### And

### Enantiomer 2 of 3-(3-(1-Cyano-1-(2-(2-fluoro-5-((4,6,7-trifluoro-1H-indol-5-yl)oxy)phenyl)-1H-imidazol-5-yl)ethyl-2,2,2-d3)phenyl)propanoic acid (64B)

### Absolute Configurations Unknown

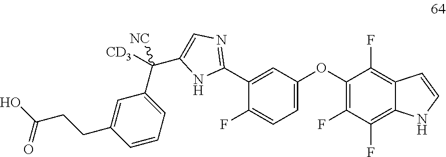 [SMILES: [2H]C([2H])([2H])C(C#N)(c1cccc(CCC(=O)O)c1)c1cnc(-c2cc(Oc3c(F)c(F)c4[nH]ccc4c3F)ccc2F)[nH]1]

### A. Enantiomer 1 and 2 of ethyl 3-(3-(1-cyano-1-(2-(2-fluoro-5-((4,6,7-trifluoro-1H-indol-5-yl)oxy)phenyl)-1H-imidazol-5-yl)ethyl-2,2,2-d3)phenyl)propanoate

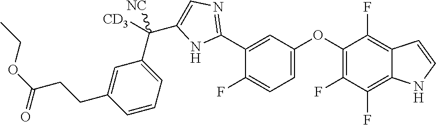 [SMILES: [2H]C([2H])([2H])C(C#N)(c1cccc(CCC(=O)OCC)c1)c1cnc(-c2cc(Oc3c(F)c(F)c4[nH]ccc4c3F)ccc2F)[nH]1]

The racemic title compound was prepared by adapting the 12-step synthesis described for Example 48. The following changes were made to the route: iodomethane was exchanged for iodomethane-d₃ in step A, 2-fluoro-5-((6-fluoro-4-(methylthio)-1H-indol-5-yl)oxy)benzimidamide (Intermediate 24-4) was exchanged for 2-fluoro-5-((4,6,7-trifluoro-1H-indol-5-yl)oxy)benzimidamide (intermediate 24-17) in step E and the last two steps (steps K and L, which call for, respectively, the oxidation of an indole 4-methylthio group to a methylsulfonyl and the hydrolysis of an ethyl propanoate substituent to a propanoic acid) were omitted. Using this modified, 10-step synthesis, the racemate was obtained as a white solid (0.316 g). This material was separated into its component enantiomers by chiral preparative HPLC (20×250 mm×10 μm CHIRALCEL OJ column; flow rate: 50 mL/min; column temperature: 35° C.; eluant: 80:20 n-hexane/ethanol with 0.1% diethylamine additive).

Enantiomer 1 (first eluting isomer) of the title compound was obtained as a white solid (0.080 g, 51%). ¹H NMR (400 MHz, CD₃OD) δ 7.59-7.58 (m, 1H), 7.38 (d, J=3.2 Hz, 1H), 7.32-7.18 (m, 5H), 7.10 (s, 1H), 7.02 (dt, J=9.2, 3.6 Hz, 1H), 6.63 (t, J=3.2 Hz, 1H), 4.05 (q, J=7.2 Hz, 2H), 2.92 (t, J=7.2 Hz, 2H), 2.60 (t, J=7.2 Hz, 2H), 1.16 (t, J=7.2 Hz, 3H) ppm. MS: 580 m/z [M+H]⁺.

Enantiomer 2 (second eluting isomer) of the title compound was obtained as a white solid (0.070 g, 44%). ¹H NMR (400 MHz, CD₃OD) δ 7.59 (dd, J=6.0, 3.2 Hz, 1H), 7.38 (d, J=3.2 Hz, 1H), 7.32-7.18 (m, 5H), 7.11 (s, 1H), 7.02 (dt, J=9.2, 3.6 Hz, 1H), 6.63 (t, J=3.2 Hz, 1H), 4.05 (q, J=7.2 Hz, 2H), 2.92 (t, J=7.2 Hz, 2H), 2.60 (t, J=7.2 Hz, 2H), 1.15 (t, J=7.2 Hz, 3H) ppm. MS: 580 m/z [M+H]⁺.

### B. Enantiomers of 3-(3-(1-cyano-1-(2-(2-fluoro-5-((4,6,7-trifluoro-1H-indol-5-yl)oxy)phenyl)-1H-imidazol-5-yl)ethyl-2,2,2-d₃)phenyl)propanoic acid

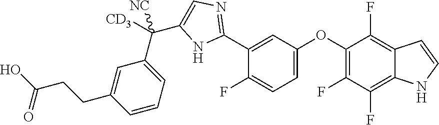 [SMILES: [2H]C([2H])([2H])C(C#N)(c1cccc(CCC(=O)O)c1)c1cnc(-c2cc(Oc3c(F)c(F)c4[nH]ccc4c3F)ccc2F)[nH]1]

Exchanging ethyl 3-(3-(1-cyano-1-(2-(2-fluoro-5-((6-fluoro-4-(methylsulfonyl)-1H-indol-5-yl)oxy)phenyl)-1H-imidazol-5-yl)ethyl)phenyl)propanoate for the separate step A products, Enantiomer 1 and Enantiomer 2, the reaction conditions for ester hydrolysis described in step L of the Example 48 synthesis were used to prepare the title compounds (Example 64A and Example 64B, respectively).

Example 64A (from Step A, Enantiomer 1) was obtained as a white solid (68 mg, 89%). ¹H NMR (400 MHz, CD₃OD) δ 7.57 (dd, J=6.0, 3.2 Hz, 1H), 7.38 (d, J=3.2 Hz, 1H), 7.36 (s, 1H), 7.31-7.21 (m, 4H), 7.09 (s, 1H), 7.02 (dt, J=9.2, 3.2 Hz, 1H), 6.63 (t, J=3.2 Hz, 1H), 2.92 (t, J=7.6 Hz, 2H), 2.59 (t, J=7.6 Hz, 2H) ppm. MS: 552 m/z [M+H]⁺.

Example 64B (from Step A, Enantiomer 2) was obtained as a white solid (60 mg, 90%). ¹H NMR (400 MHz, CD₃OD) δ 7.57 (dd, J=6.0, 3.2 Hz, 1H), 7.38 (d, J=3.2 Hz, 1H), 7.37 (s, 1H), 7.31-7.21 (m, 4H), 7.08 (s, 1H), 7.02 (dt, J=8.8, 3.6 Hz, 1H), 6.64 (t, J=2.8 Hz, 1H), 2.93 (t, J=7.6 Hz, 2H), 2.59 (t, J=7.6 Hz, 2H) ppm. MS: 552 m/z [M+H]⁺.

The following enantiomeric Example compounds were prepared by a synthetic route analogous to that described for Example 64.

| | | | | LCMS | MS |
|---|---|---|---|---|---|
| Structure | Ex. No. | Name | ¹H NMR | Rₜ (min) | (m/z) |
| 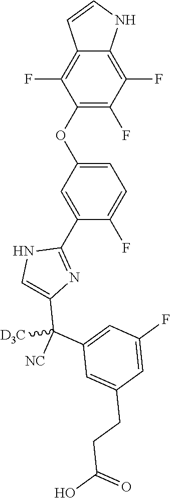 [SMILES: [2H]C([2H])([2H])C(C#N)(c1cc(F)cc(CCC(=O)O)c1)c1c[nH]c(-c2cc(Oc3c(F)c(F)c4[nH]ccc4c3F)ccc2F)n1] | 65 | Enantiomer 1 of 3-(3-(1- cyano-1-(2-(2-fluoro-5- ((4,6,7-trifluoro-1H-indol-5- yl)oxy)phenyl)-1H-imidazol- 4-yl)ethyl-2,2,2-d₃)-5- fluorophenyl)propanoic acid | (400 MHz, CD3OD) δ 7.56 (dd, J = 2.8, 5.6 Hz, 1H), 7.39 (d, J = 2.8 Hz, 1H), 7.31-7.25 (m, 2H), 7.21 (s, 1 H), 7.10-7.00 (m, 3 H), 6.64 (t, J = 2.8 Hz, 1H), 2.93 (t, J = 7.6 Hz, 2H), 2.61 (t, J = 7.6 Hz, 2H) ppm. | | 570 [M + H]⁺ |
| 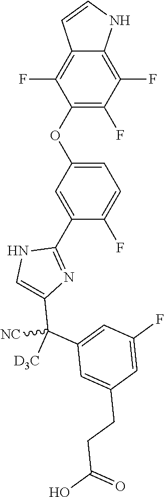 [SMILES: [2H]C([2H])([2H])C(C#N)(c1cc(F)cc(CCC(=O)O)c1)c1c[nH]c(-c2cc(Oc3c(F)c(F)c4[nH]ccc4c3F)ccc2F)n1] | 66 | Enantiomer 2 of 3-(3-(1- cyano-1-(2-(2-fluoro-5- ((4,6,7-trifluoro-1H-indol-5- yl)oxy)phenyl)-1H-imidazol- 4-yl)ethyl-2,2,2-d₃)-5- fluorophenyl)propanoic acid | (400 MHz, CD3OD) δ 7.56 (dd, J = 3.0, 6.0 Hz, 1H), 7.38 (d, J = 2.8 Hz, 1H), 7.28-7.23 (m, 2H), 7.21 (s, 1H), 7.07-6.99 (m, 3H), 6.63 (t, J = 2.8 Hz, 1H), 2.93 (t, J = 7.6 Hz, 2H), 2.61 (t, J = 7.6 Hz, 2H) ppm | | 570 [M + H]⁺ |

### Example 67A

### Enantiomer 1 of 3-(3-(1-cyano-1-(2-(2-fluoro-5-((4,6,7-trifluoro-1H-indol-5-yl-3-d)oxy)phenyl)-1H-imidazol-4-yl)ethyl-2,2,2-d3)-5-fluorophenyl)propanoic acid

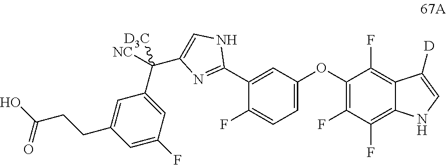 [SMILES: [2H]c1c[nH]c2c(F)c(F)c(Oc3ccc(F)c(-c4nc(C(C#N)(c5cc(F)cc(CCC(=O)O)c5)C([2H])([2H])[2H])c[nH]4)c3)c(F)c12]

To a stirred solution of Example 65 (Enantiomer 1 of 3-(3-(1-cyano-1-(2-(2-fluoro-5-((4,6,7-trifluoro-1H-indol-5-yl)oxy)phenyl)-1H-imidazol-4-yl)ethyl-2,2,2-d₃)-5-fluorophenyl)propanoic acid; 65.0 mg, 0.114 mmol) in acetone-d₆ (1 mL) was added a mixture of 0.1% (w/w) sulfuric acid in deuterated water (6 mL). The mixture was stirred for three days at 100° C. and then cooled to room temperature. The resulting suspension was filtered and the collected solid was dried to afford the title compound as a white solid (26.6 mg, 41%). ¹H NMR (400 MHz, CD₃OD) δ 7.57 (dd, J=3.2, 5.6 Hz, 1H), 7.38 (s, 1H), 7.26-7.21 (m, 2H), 7.16 (s, 1H), 7.04-6.96 (m, 3H), 2.93 (t, J=7.6 Hz, 2H), 2.59 (t, J=7.6 Hz, 2H) ppm. MS: 571 m/z [M+H]⁺.

### Example 67B

### Enantiomer 2 of 3-(3-(1-cyano-1-(2-(2-fluoro-5-((4,6,7-trifluoro-1H-indol-5-yl-3-d)oxy)phenyl)-1H-imidazol-4-yl)ethyl-2,2,2-d3)-5-fluorophenyl)propanoic acid

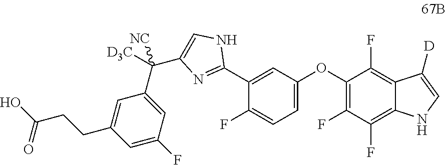 [SMILES: [2H]c1c[nH]c2c(F)c(F)c(Oc3ccc(F)c(-c4nc(C(C#N)(c5cc(F)cc(CCC(=O)O)c5)C([2H])([2H])[2H])c[nH]4)c3)c(F)c12]

Exchanging Example 65 for its enantiomer (Example 66), the same procedure described in Example 65 was used to prepared the title compound as a white solid (34.3 mg, 49%). ¹H NMR (400 MHz, CD₃OD) δ 7.58-7.56 (m, 1H), 7.38 (s, 1H), 7.26-7.21 (m, 2H), 7.16 (s, 1H), 7.04-6.97 (m, 3H), 2.93 (d, J=7.6 Hz, 2H), 2.59 (d, J=7.6 Hz, 2H) ppm. MS-ESI m/z: 571 m/z [M+H]⁺.

### Example 68

### 3-(3-(2-(2-(2-Fluoro-5-((6-fluoro-4-(methylsulfonyl)-1H-indol-5-yl)oxy)phenyl)-1H-imidazol-5-yl)-1-hydroxypropan-2-yl)phenyl)propanoic acid

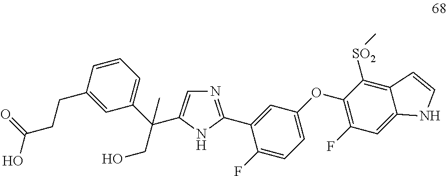 [SMILES: CC(CO)(c1cccc(CCC(=O)O)c1)c1cnc(-c2cc(Oc3c(F)cc4[nH]ccc4c3S(C)(=O)=O)ccc2F)[nH]1]

### A. 2-(2-(2-Fluoro-5-((6-fluoro-4-(methylsulfonyl)-1H-indol-5-yl)oxy)phenyl)-1H-imidazol-5-yl)-2-(3-iodophenyl)propyl acetate

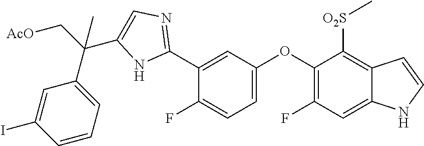 [SMILES: CC(=O)OCC(C)(c1cccc(I)c1)c1cnc(-c2cc(Oc3c(F)cc4[nH]ccc4c3S(C)(=O)=O)ccc2F)[nH]1]

To a solution of 2-(2-(2-fluoro-5-((6-fluoro-4-(methylthio)-1H-indol-5-yl)oxy)phenyl)-1H-imidazol-5-yl)-2-(3-iodophenyl)propyl acetate (500 mg, 0.76 mmol, Example 48, Step E product) in methanol (10 mL) was added a solution of ammonium molybdate tetrahydrate (0.100 g, 80.9 μmol) in 1 mL of 30% aqueous hydrogen peroxide. The reaction was stirred at room temperature for two hours and then diluted with ethyl acetate (40 mL). This solution was washed with aqueous sodium sulfite solution (2×30 mL) and brine (1×30 mL), dried over sodium sulfate and concentrated. The residue was purified by flash chromatography over silica (50% petroleum ether in 1:1 ethyl acetate/dichloromethane) to afford the title compound as a colorless solid (0.260 g, 50%). MS: 692 m/z [M+H]⁺.

### B. Ethyl 3-(3-(1-acetoxy-2-(2-(2-fluoro-5-((6-fluoro-4-(methylsulfonyl)-1H-indol-5-yl)oxy)phenyl)-1H-imidazol-5-yl)propan-2-yl)phenyl)propanoate

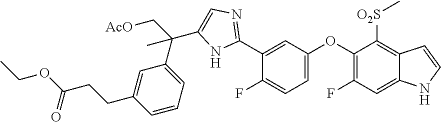 [SMILES: CCOC(=O)CCc1cccc(C(C)(COC(C)=O)c2cnc(-c3cc(Oc4c(F)cc5[nH]ccc5c4S(C)(=O)=O)ccc3F)[nH]2)c1]

To a stirred solution of step A product (0.260 g, 0.38 mmol), 3,3-diethoxyprop-1-ene (147 mg, 1.13 mmol), tetrabutylammonium chloride (105 mg, 0.376 mmol) in DMF (5 mL) was added tributylamine (139 mg, 0.750 mmol) and Pd(OAc)₂ (9.0 mg, 0.04 mmol). The mixture was heated at 90° C. for three hours and then cooled to room temperature and diluted with ethyl acetate (150 mL). This solution was washed with water (2×20 mL) and brine (3×20 mL), dried over sodium sulfate and concentrated. The residue was purified by flash chromatography over silica (45% petroleum ether in 1:1 ethyl acetate/dichloromethane) to afford the title compound as a yellow solid (0.120 g, 50%). MS: 666 m/z [M+H]⁺.

### C. Example 68, 3-(3-(2-(2-(2-Fluoro-5-((6-fluoro-4-(methylsulfonyl)-1H-indol-5-yl)oxy)phenyl)-1H-imidazol-5-yl)-1-hydroxypropan-2-yl)phenyl)propanoic acid

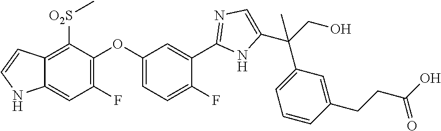 [SMILES: CC(CO)(c1cccc(CCC(=O)O)c1)c1cnc(-c2cc(Oc3c(F)cc4[nH]ccc4c3S(C)(=O)=O)ccc2F)[nH]1]

To a stirred solution of step B product (0.120 g, 0.180 mmol) in THF (7 mL) was added a solution of lithium hydroxide (32.0 mg, 1.35 mmol) in water (0.7 mL). The reaction mixture was stirred overnight at room temperature and then concentrated. The residue was dissolved in 2 mL of water and treated, with stirring, with 1.0 N hydrochloric acid until a pH of ˜4 was achieved. The resulting precipitate was collected by filtration and subjected to preparative HPLC purification to afford the title compound as a white solid (46 mg, 43%). ¹H NMR (400 MHz, CD₃OD) δ 7.65 (d, J=10.8 Hz, 1H), 7.57-7.53 (m, 1H), 7.52 (d, J=7.2 Hz, 1H), 7.23-7.18 (m, 4H), 7.10 (t, J=7.2 Hz, 2H), 6.99 (s, 1H), 6.98-6.93 (m, 1H), 4.13 (d, J=11.2 Hz, 1H), 3.91 (d, J=10.8 Hz, 1H), 3.33 (s, 3H), 2.89 (t, J=7.6 Hz, 2H), 2.56 (t, J=7.6 Hz, 2H), 1.68 (s, 3H) ppm. MS: 596 m/z [M+H]⁺.

The following Example compound was prepared by a synthetic route analogous to that e

| | | | | LCMS | MS |
|---|---|---|---|---|---|
| Structure | Ex. No. | Name | ¹H NMR | Rt (min) | (m/z) |
| 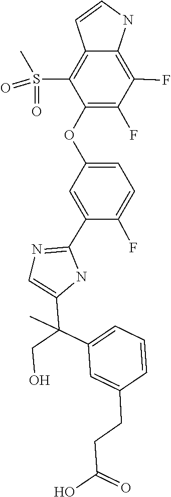 | 69 | 3-(3-(2-(2-(5-((6,7-difluoro-4- (methylsulfonyl)-1H-indol-5- yl)oxy)-2-fluorophenyl)-1H- imidazol-5-yl)-1- hydroxypropan-2- yl)phenyl)propanoic acid | (400 MHz, CD₃OD) δ 7.59-7.55 (m, 2H), 7.27-7.18 (m, 4H), 7.10-7.00 (m, 3H), 6.99 (s, 1H), 4.13 (d, J = 10.8 Hz, 1H), 3.91 (d, J = 10.8 Hz, 1H), 3.33 (s, 3H), 2.88 (t, J = 7.6 Hz, 2H), 2.56 (t, J = 7.2 Hz, 2H), 1.68 (s, 3H) ppm. | 0.76 | 614 [M + H]⁺ |

### Example 70

### rac-3-(3-((1r,3r)-1-(2-(5-((6,7-Difluoro-4-(methylsulfonyl)-1H-indol-5-yl)oxy)-2-fluorophenyl)-1H-imidazol-5-yl)-3-methoxy-3-methylcyclobutyl)phenyl)propanoic acid

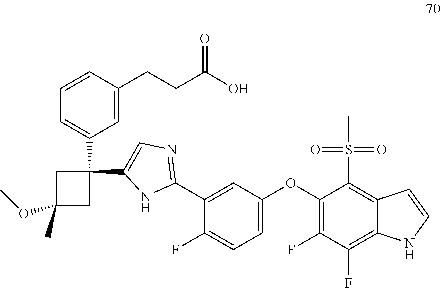 [SMILES: CO[C@]1(C)C[C@@](c2cccc(CCC(=O)O)c2)(c2cnc(-c3cc(Oc4c(F)c(F)c5[nH]ccc5c4S(C)(=O)=O)ccc3F)[nH]2)C1]

### A. 1-(3-Bromophenyl)-3,3-dimethoxycyclobutane-1-carbonitrile

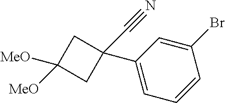 [SMILES: COC1(OC)CC(C#N)(c2cccc(Br)c2)C1]

To a stirred and cooled (0° C.) suspension of 2-(3-bromophenyl)acetonitrile (20.00 g, 103.1 mmol) in DMF (200 mL) was added sodium hydride (7.40 g, 309 mmol). After stirring the frothy mixture for one hour, 1,3-dibromo-2,2-dimethoxypropane (27.00 g, 103.1 mmol) was slowly added. The reaction was brought to 100° C. and continued at this temperature overnight. The mixture was then cooled to room temperature and concentrated. The residue was diluted with water (200 mL) and, while stirring, treated with 6 N hydrochloric acid until a pH of 2-3 was achieved. The suspension was extracted with ethyl acetate (3×˜100 mL) and the combined extracts were washed with water (1×200 mL) and brine (1×200 mL), dried over magnesium sulfate and concentrated. The residue was purified by flash chromatography over silica to afford the title compound as a white solid (18.00 g, 59%). MS: 296 m/z [M+H]⁺.

### B. 1-(3-Bromophenyl)-3-oxocyclobutane-1-carbonitrile

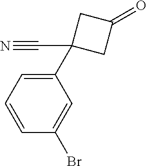 [SMILES: N#CC1(c2cccc(Br)c2)CC(=O)C1]

To a stirred solution of step A product (2.70 g, 9.12 mmol) in acetone (21 mL) was added 6 M hydrochloric acid (15 mL). The reaction was stirred at room temperature for six hours and then diluted with ethyl acetate (120 mL). This solution was washed with water (2×45 mL) and brine (1×45 mL), dried over sodium sulfate and concentrated. The residue was purified by flash chromatography over silica (0-20% ethyl acetate in petroleum ether) to afford the title compound as a white solid (1.80 g, 79%).

### C. (1r,3r)-1-(3-Bromophenyl)-3-hydroxy-3-methylcyclobutane-1-carbonitrile and (1s,3s)-1-(3-bromophenyl)-3-hydroxy-3-methylcyclobutane-1-carbonitrile

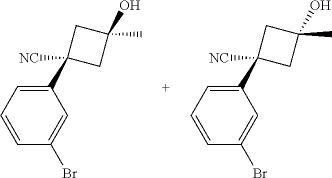

To a stirred and cooled (0° C.) solution of 1-(3-bromophenyl)-3-oxocyclobutane-1-carbonitrile (1.80 g, 7.20 mmol) in THF (20 mL) was added a 1.0 M solution of methylmagnesium bromide in THF (14.4 mL, 14.4 mmol). The mixture was stirred at 0° C. for one hour and then warmed to room temperature and stirred for another four hours. After this time, the reaction was quenched with the addition of water (100 mL with the first 1 mL added dropwise). The resulting suspension was made acidic (pH 2-3) by adding 1.0 N hydrochloric acid and then extracted with ethyl acetate (3×70 mL). The combined extracts were washed with water (1×100 mL) and brine (1×100 mL), dried over magnesium sulfate and concentrated. The residue was purified by flash chromatography over silica (5-20% ethyl acetate in cyclohexane) to provide the separated title compounds. (1r,3r)-1-(3-Bromophenyl)-3-hydroxy-3-methylcyclobutane-1-carbonitrile and (1s,3s)-1-(3-bromophenyl)-3-hydroxy-3-methylcyclobutane-1-carbonitrile were both afforded as light yellow oils (0.800 g and 0.700 g, respectively, corresponding to yields of 42% and 30%).

### D. (1r,3r)-1-(3-Bromophenyl)-3-methoxy-3-methylcyclobutane-1-carbonitrile

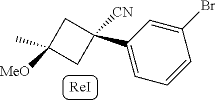

To a stirred and cooled (0° C.) suspension of (1r,3r)-1-(3-bromophenyl)-3-hydroxy-3-methylcyclobutane-1-carbonitrile (0.800 g, 2.86 mmol) in DMF (10 mL) was added sodium hydride (0.200 g, 8.33 mmol). After one hour, the mixture was allowed to warm to room temperature and then treated with methyl iodide (0.28 mL, 4.5 mmol). The reaction was stirred overnight at room temperature and then quenched with the addition of water (100 mL with the first 1 mL added dropwise). After acidifying (pH 2-3) the suspension with the addition of 1.0 N hydrochloric acid, it was extracted with ethyl acetate (3×50 mL). The combined extracts were washed with water (1×˜75 mL) and brine (1×75 mL), dried over magnesium sulfate and concentrated. The residue was purified by flash chromatography over silica (10% ethyl acetate in petroleum ether) to afford the title compound as a white solid (0.700 g, 87%). MS: 280, 282 m/z [M+H]⁺.

### E. (1r,3r)-1-(3-Bromophenyl)-3-methoxy-3-methylcyclobutane-1-carboxylic acid

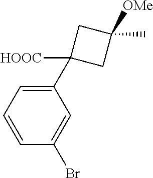

To a stirred suspension of step D product (2.00 g, 7.17 mmol) in a mixture of methanol (20 mL) and water (5 mL) was added potassium hydroxide (8.05 g, 143 mmol). The reaction was stirred at 80° C. for 24 hours and then concentrated to remove the organic solvent. The residue was taken up in water (80 mL) and this stirred solution was made acidic (pH 2-3) with the addition of 6 N hydrochloric acid. The resulting suspension was extracted with ethyl acetate (3×50 mL). The combined extracts were washed with water (1×100 mL) and brine (1×100 mL), dried over magnesium sulfate and concentrated. The residue was purified by chromatography (5-20% ethyl acetate in cyclohexane) to afford the title compound as a light yellow solid (2.10 g, 98%). MS: 321, 323 m/z [M+Na]⁺.

### F. (1r,3r)-3-Methoxy-1-(3-((E)-3-methoxy-3-oxoprop-1-en-1-yl)phenyl)-3-methylcyclobutane-1-carboxylic acid

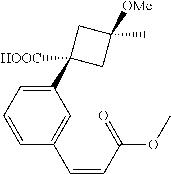 [SMILES: COC(=O)/C=C\c1cccc([C@]2(C(=O)O)C[C@](C)(OC)C2)c1]

To a stirred suspension of step E product (1.93 g, 6.48 mmol) in DMF (30 mL) was added methyl acrylate (1.67 g, 19.4 mmol), p(o-tol)₃ (0.590 g, 1.94 mmol), triethylamine (2.70 mL, 19.4 mmol) and Pd(OAc)₂ (0.291 g, 1.30 mmol). The reaction was heated at 100° C. for four hours and then cooled to room temperature and diluted with water (150 mL). The stirred mixture was made acidic (pH ˜3) with the addition of 2 N hydrochloric acid and extracted with ethyl acetate (3×80 mL). The combined extracts were dried over magnesium sulfate and concentrated. The residue was purified by flash chromatography over silica (50% ethyl acetate in petroleum ether) to afford the title compound as a colorless oil (0.960 g, 49%). MS: 305 m/z [M+H]⁺.

### G. (1r,3r)-3-Methoxy-1-(3-(3-methoxy-3-oxopropyl)phenyl)-3-methylcyclobutane-1-carboxylic acid

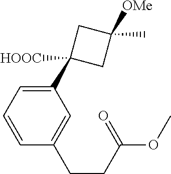 [SMILES: COC(=O)CCc1cccc([C@]2(C(=O)O)C[C@](C)(OC)C2)c1]

A stirred suspension of step F product (0.960 g, 3.16 mmol) and 10% Pd/C (0.200 g) in ethyl acetate (50 mL) was cycled between vacuum and a nitrogen atmosphere three times. The reaction vessel was evacuated a final time and then backfilled with hydrogen (via balloon). The reaction was stirred for two hours and then filtered through a pad of Celite pad, which was subsequently washed with additional ethyl acetate (˜50 mL, total). The combined filtrate was concentrated to afford the crude title compound as a colorless oil (0.880 g, 92%). MS: 329 m/z [M+Na]⁺.

### H. Methyl 3-(3-((1r,3r)-1-(2-bromoacetyl)-3-methoxy-3-methylcyclobutyl)phenyl)propanoate

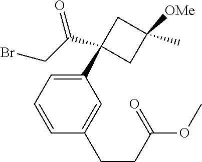 [SMILES: COC(=O)CCc1cccc([C@]2(C(=O)CBr)C[C@](C)(OC)C2)c1]

To a stirred and cooled (0° C.) solution of step G product (0.880 g, 2.88 mmol) in dichloromethane (15 mL) was added oxalyl chloride (1.46 g, 11.5 mmol) followed by one drop of DMF. The reaction was allowed to warm to room temperature, stirred for another one hour and then concentrated. The residue was co-evaporated once with heptane (15 mL) before taking the crude acid chloride up in 1:1 THF/acetonitrile (10 mL). To this stirred and cooled (0° C.) solution was added, dropwise over five minutes, a 2.0 M solution of trimethylsilyldiazomethane in hexanes (14.4 mL, 28.8 mmol). The reaction was allowed to warm to room temperature and stirring was continued overnight. After this time, the mixture was returned to 0° C. and treated, dropwise over 10 minutes, with a 32% solution of hydrobromic acid in acetic acid (5.30 mL, 28.8 mmol; vigorous gas evolution observed). The mixture was stirred for 30 minutes before partitioning between brine (75 mL) and ethyl acetate (50 mL). The organic layer was combined with a second extract (ethyl acetate, 1×50 mL), dried over magnesium sulfate and concentrated. The residue was purified by flash chromatography over silica (20% ethyl acetate in petroleum ether) to afford the title compound as a brown oil (0.835 g, 76%). MS: 383, 385 m/z [M+H]⁺.

### I. Methyl 3-(3-((1r,3r)-1-(2-(5-((6,7-difluoro-4-(methylthio)-1H-indol-5-yl)oxy)-2-fluorophenyl)-1H-imidazol-5-yl)-3-methoxy-3-methylcyclobutyl)phenyl)propanoate

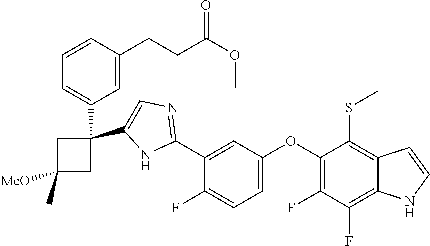 [SMILES: COC(=O)CCc1cccc([C@]2(c3cnc(-c4cc(Oc5c(F)c(F)c6[nH]ccc6c5SC)ccc4F)[nH]3)C[C@](C)(OC)C2)c1]

To a stirred solution of step H product (0.135 g, 0.352 mmol) in DMF (10 mL) was added 5-((6,7-difluoro-4-(methylthio)-1H-indol-5-yl)oxy)-2-fluorobenzimidamide (0.136 g, 0.389 mmol, Intermediate 24-8) and potassium carbonate (0.098 g, 0.707 mmol). The reaction was heated at 100° C. for two hours and then cooled to room temperature and diluted with water (100 mL). The resulting suspension was extracted with ethyl acetate (3×30 mL) and the combined extracts were washed with brine (1×100 mL), dried over sodium sulfate and concentrated. The residue was purified by preparative HPLC to afford the title compound as a yellow solid (0.105 g, 46%). MS: 636 m/z [M+H]⁺.

### J. Methyl 3-(3-((1r,3r)-1-(2-(5-((6,7-difluoro-4-(methylsulfonyl)-1H-indol-5-yl)oxy)-2-fluorophenyl)-1H-imidazol-5-yl)-3-methoxy-3-methylcyclobutyl)phenyl)propanoate

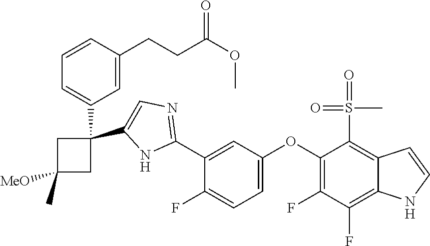 [SMILES: COC(=O)CCc1cccc([C@]2(c3cnc(-c4cc(Oc5c(F)c(F)c6[nH]ccc6c5S(C)(=O)=O)ccc4F)[nH]3)C[C@](C)(OC)C2)c1]

To a stirred solution of step I product (0.105 g, 0.165 mmol) in methanol (10 mL) was added a mixture of ammonium molybdate tetrahydrate (0.200 g, 0.162 mmol) in 30% aqueous hydrogen peroxide (1 mL). The reaction was stirred overnight at room temperature and then diluted with water (50 mL) and extracted with ethyl acetate (3×30 mL). The combined extracts were washed with aqueous sodium sulfite solution (1×30 mL) and brine (1×30 mL), dried over sodium sulfate and concentrated. The residue was purified by preparative HPLC to afford the title compound as give rac-methyl 3-(3-((1r,3r)-1-(2-(5-((6,7-difluoro-4-(methylsulfonyl)-1H-indol-5-yl)oxy)-2-fluorophenyl)-1H-imidazol-5-yl)-3-methoxy-3-methylcyclobutyl)phenyl)propanoate as pale yellow solid (0.100 g, 91%). MS: 668 m/z [M+H]⁺.

### K. Example 70, 3-(3-((1r,3r)-1-(2-(5-((6,7-Difluoro-4-(methylsulfonyl)-1H-indol-5-yl)oxy)-2-fluorophenyl)-1H-imidazol-5-yl)-3-methoxy-3-methylcyclobutyl)phenyl)propanoic acid

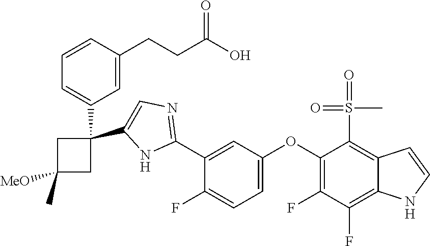 [SMILES: CO[C@]1(C)C[C@@](c2cccc(CCC(=O)O)c2)(c2cnc(-c3cc(Oc4c(F)c(F)c5[nH]ccc5c4S(C)(=O)=O)ccc3F)[nH]2)C1]

To a stirred solution of step J product (0.100 g, 0.150 mmol) in a mixture of THF (5 mL) and water (1 mL) was added lithium hydroxide monohydrate (63.0 mg, 1.50 mmol). The reaction mixture was stirred overnight at room temperature and then concentrated. The residue was taken up in water (10 mL) and the resulting stirred solution was made acidic (pH˜2) with the addition of 1.0 N hydrochloric acid. The precipitate which formed was collected by filtration and purified by preparative HPLC to afford the title compound as a white solid (36.0 mg, 37%). ¹H NMR (400 MHz, CD₃OD) δ 7.59-7.58 (m, 1H), 7.56 (dd, J=3.2, 5.6 Hz, 1H), 7.26 (t, J=2.8 Hz, 1H), 7.23-7.16 (m, 3H), 7.12 (d, J=8.0 Hz, 1H), 7.03-6.98 (m, 3H), 3.34 (s, 3H), 3.18 (s, 3H), 2.89 (t, J=7.6 Hz, 2H), 2.86-2.78 (m, 4H), 2.56 (t, J=7.6 Hz, 2H), 1.28 (s, 3H) ppm. MS: 654 m/z [M+H]⁺.

The following Example compound was prepared by a synthetic route analogous to that described for Example 70.

| | | | | LCMS | MS |
|---|---|---|---|---|---|
| Structure | Ex. No. | Name | ¹H NMR | Rₜ (min) | (m/z) |
| 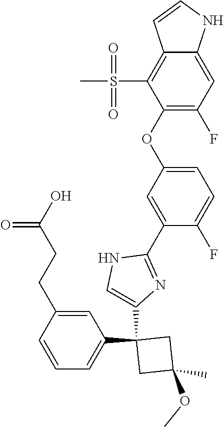 [SMILES: CO[C@]1(C)C[C@@](c2cccc(CCC(=O)O)c2)(c2c[nH]c(-c3cc(Oc4c(F)cc5[nH]ccc5c4S(C)(=O)=O)ccc3F)n2)C1] | 71 | 3-(3-((1r,3r)-1-(2-(2-fluoro-5- ((6-fluoro-4-(methylsulfonyl)- 1H-indol-5-yl)oxy)phenyl)- 1H-imidazol-4-yl)-3- methoxy-3- methylcyclobutyl)phenyl) propanoic acid | (400 MHz, DMSO-d₆) δ 7.67 (d, J = 10.0 Hz, 1H), 7.54 (d, J = 3.2 Hz, 1H), 7.50-7.48 (m, 1H), 7.36 (s, 1H), 7.30-7.23 (m, 2H), 7.21-7.20 (m, 2H), 7.15 (d, J = 7.6 Hz, 1H ), 7.07-7.05 (m, 2H), 3.37 (s, 3H), 3.19 (s, 3H), 2.90 (t, J = 7.6 Hz, 3H), 2.83 (s, 3H), 2.58 (t, J = 8.0 Hz, 2H), 1.31 (s, 3H). | 0.82 | 636 [M + H]⁺ |

### Example 72

### 3-(3-Fluoro-5-((1r,3r)-1-(2-(2-fluoro-5-((6-fluoro-4-(methylsulfonyl)-1H-indol-5-yl)oxy)phenyl)-1H-imidazol-5-yl)-3-methoxy-3-methylcyclobutyl)phenyl)propanoic acid

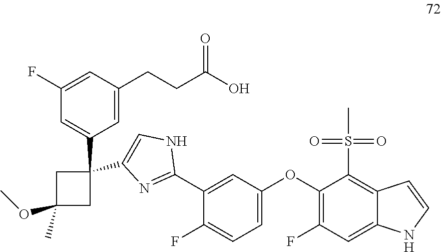 [SMILES: CO[C@]1(C)C[C@@](c2cc(F)cc(CCC(=O)O)c2)(c2c[nH]c(-c3cc(Oc4c(F)cc5[nH]ccc5c4S(C)(=O)=O)ccc3F)n2)C1]

### A. 1-(3-Bromo-5-fluorophenyl)-3-hydroxycyclobutane-1-carboxylic acid

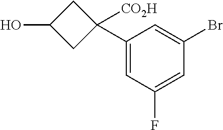 [SMILES: O=C(O)C1(c2cc(F)cc(Br)c2)CC(O)C1]

To a stirred 2.0 M solution of isopropylmagnesium chloride in THF (61.6 mL, 123.2 mmol) was added, dropwise over 15 minutes, a solution of 2-(3-bromo-5-fluorophenyl)acetonitrile (15.8 g, 72.5 mmol) in THF (100 mL). The reaction was maintained at 30-40° C. for one hour and then treated, dropwise, with (+/−)-epichlorohydrin (12.1 g, 130.8 mmol). The reaction was stirred for another 3.5 hours, cooled to room temperature, and treated with an additional portion of 2.0 M isopropylmagnesium chloride solution (47.0 mL, 94.0 mmol). The mixture was then stirred overnight at 30° C. After this time, the reaction was cooled to 0° C. and quenched by the slow addition of 5.0 N hydrochloric acid (quantity sufficient to achieve pH˜2). The cooling bath was then removed and the mixture was allowed to warm to room temperature, stirred for 20 minutes and extracted with ethyl acetate (3×100 mL). The combined extracts were dried over magnesium sulfate and concentrated. The residue was purified by flash chromatography over silica (40% ethyl acetate in petroleum ether) to provide the title compound as a white solid (4.01 g, 19%). MS: 289 m/z [M+H]⁺.

### B. Methyl 1-(3-bromo-5-fluorophenyl)-3-hydroxycyclobutane-1-carboxylate

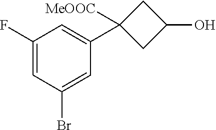 [SMILES: COC(=O)C1(c2cc(F)cc(Br)c2)CC(O)C1]

To a stirred solution of step A product (8.00 g, 27.7 mmol) in methanol (100 mL) was slowly added concentrated sulfuric acid (5 mL). The mixture was heated at 50° C. for two hours and then concentrated. The residue was partitioned between water (200 mL) and ethyl acetate (100 mL). The organic layer was combined with additional extracts (ethyl acetate, 2×100 mL), washed with water (1×150 mL) and brine (1×150 mL) and dried over magnesium sulfate. The solution was concentrated to afford the crude title compound as a light yellow solid (8.02 g, 95%). MS: 303, 305 m/z [M+H]⁺.

### C. Methyl 1-(3-bromo-5-fluorophenyl)-3-oxocyclobutane-1-carboxylate

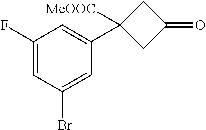 [SMILES: COC(=O)C1(c2cc(F)cc(Br)c2)CC(=O)C1]

To a stirred solution of step B product (8.00 g, 26.5 mmol) in dichloromethane (200 mL) was added TEMPO (0.827 g, 5.03 mmol) and (diacetoxyiodo)benzene (12.8 g, 39.7 mmol). After two hours at room temperature, the mixture was concentrated. The residue was purified by flash chromatography over silica (25% ethyl acetate in petroleum ether). The title compound was obtained as light red oil (7.03 g, 88%). MS: 301, 303 m/z [M+H]⁺.

### D. 1-(3-Bromo-5-fluorophenyl)-3-oxocyclobutane-1-carboxylic acid

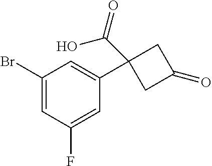 [SMILES: O=C1CC(C(=O)O)(c2cc(F)cc(Br)c2)C1]

To a stirred solution step C product (7.03 g, 23.3 mmol) in a mixture of THF (100 mL) and water (10 mL) was added lithium hydroxide monohydrate (4.90 g, 117 mmol). The reaction mixture was stirred at 30° C. for three hours and then concentrated. The residue was taken up in water (200 mL) and the solution was stirred with a sufficient quantity of 2.0 N hydrochloric acid to achieve pH˜3. The resulting suspension was extracted with ethyl acetate (3×100 mL) and the combined extracts were dried over magnesium sulfate and concentrated. Crude title compound was afforded as an off-white solid (6.04 g, 90%). MS: 287, 289 m/z [M+H]⁺.

### E. (1s,3s)-1-(3-Bromo-5-fluorophenyl)-3-hydroxy-3-methylcyclobutane-1-carboxylic acid and (1r,3r)-1-(3-bromo-5-fluorophenyl)-3-hydroxy-3-methylcyclobutane-1-carboxylic acid

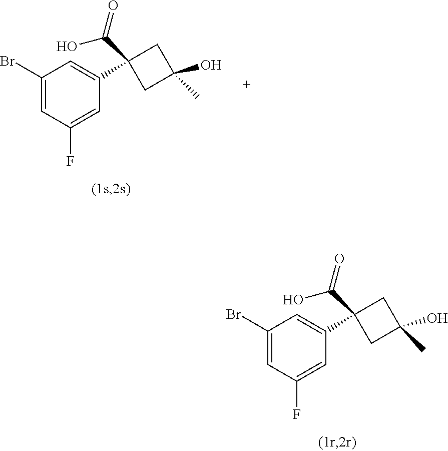 [SMILES: C[C@]1(O)C[C@](C(=O)O)(c2cc(F)cc(Br)c2)C1 ; C[C@]1(O)C[C@@](C(=O)O)(c2cc(F)cc(Br)c2)C1]

To a stirred and cooled (−78° C.) solution of step D product (7.00 g, 24.5 mmol) in THF (100 mL) was added, dropwise over 15 minutes, a 3.0 M solution of methylmagnesium bromide in diethyl ether (24.5 mL, 73.5 mmol). The reaction was stirred at −78° C. for another one hour and then quenched by the addition of a saturated ammonium chloride solution (˜20 mL total, with first 1-2 mL introduced dropwise). The mixture was allowed to warm to room temperature and diluted with water (100 mL). This stirred suspension was adjusted to pH˜3 with the addition of 3.0 N hydrochloric acid and then extracted with ethyl acetate (3×100 mL). The combined extracts were dried over magnesium sulfate and concentrated. The residue was purified by flash chromatography over silica (30% ethyl acetate in petroleum ether) to obtain the separated, title compounds. The (1r,3r)- and (1s,3s)-isomers were both afforded as colorless oils (2.40 g and 1.94 g, respectively, corresponding to yields of 42% and 30%). MS (both products) 285, 287 m/z [M−OH⁺].

### F. (1r,3r)-1-(3-Bromo-5-fluorophenyl)-3-methoxy-3-methylcyclobutane-1-carboxylic acid

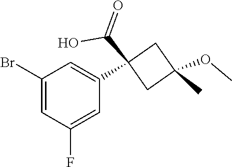 [SMILES: CO[C@]1(C)C[C@](C(=O)O)(c2cc(F)cc(Br)c2)C1]

To a stirred and cooled (0° C.) solution of (1r,3r)-1-(3-bromo-5-fluorophenyl)-3-hydroxy-3-methylcyclobutane-1-carboxylic acid (0.625 g, 2.06 mmol) in THF (20 mL) was added a 60% dispersion of sodium hydride in mineral oil (0.432 g, 10.8 mmol). The frothy mixture was stirred at 0° C. for another 30 minutes before adding, dropwise over 3-4 minutes, methyl iodide (0.67 mL, 10.8 mmol). The reaction was then allowed to warm to room temperature and then stirred for four hours before, successively, diluting with water (50 mL) and acidifying to pH˜3 with 5.0 N hydrochloric acid. The resulting suspension was extracted with ethyl acetate (3×30 mL) and the combined extracts were dried over magnesium sulfate and concentrated. The residue was purified by flash column chromatography over silica (30% ethyl acetate in petroleum ether) to afford the title compound as a colorless oil (0.500 g, 77%). MS: 317, 319 m/z [M+H]⁺.

### G. Example 72, 3-(3-Fluoro-5-((1r,3r)-1-(2-(2-fluoro-5-((6-fluoro-4-(methylsulfonyl)-1H-indol-5-yl)oxy)phenyl)-1H-imidazol-5-yl)-3-methoxy-3-methylcyclobutyl)phenyl)propanoic acid

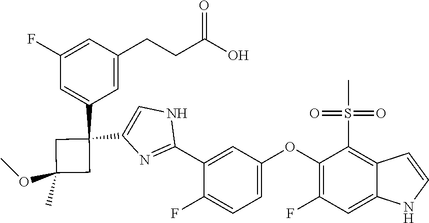 [SMILES: CO[C@]1(C)C[C@@](c2cc(F)cc(CCC(=O)O)c2)(c2c[nH]c(-c3cc(Oc4c(F)cc5[nH]ccc5c4S(C)(=O)=O)ccc3F)n2)C1]

Exchanging (1r,3r)-3-methoxy-1-(3-(3-methoxy-3-oxopropyl)phenyl)-3-methylcyclobutane-1-carboxylic acid for step F product, procedures analogous to those described in steps H-K of the Example 70 synthesis were used to prepare the title compound as a white solid. ¹H NMR (400 MHz, CD₃OD) δ 7.66 (d, J=10.8 Hz, 1H), 7.56 (dd, J=6.0, 3.2 Hz, 1H), 7.53 (d, J=3.6 Hz, 1H), 7.24-7.17 (m, 2H), 7.09 (s, 1H), 6.99-6.92 (m, 2H), 6.88 (d, J=9.6 Hz, 1H), 6.39 (s, 1H-1), 3.33 (s, 3H-1), 3.27 (s, 3H-1), 2.97-2.92 (m, 4H), 2.73 (d, J=12.8 Hz, 2H), 2.62 (t, J=7.6 Hz, 2H), 1.23 (s, 3H) ppm. MS: 654 m/z [M+H]⁺.

The following Example compounds were prepared by synthetic routes analogous to that described for Example 72.

| | | | | LCMS | LCMS |
|---|---|---|---|---|---|
| Structure | Ex. No. | Name | ¹H NMR | Rₜ (min) | (m/z) |
| 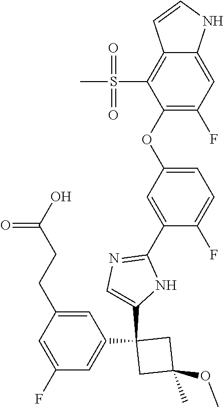 [SMILES: CO[C@]1(C)C[C@](c2cc(F)cc(CCC(=O)O)c2)(c2cnc(-c3cc(Oc4c(F)cc5[nH]ccc5c4S(C)(=O)=O)ccc3F)[nH]2)C1] | 73 | 3-(3-fluoro-5-((1s,3s)-1-(2- (2-fluoro-5-((6-fluoro-4- (methylsulfonyl)-1H-indol- 5-yl)oxy)phenyl)-1H- imidazol-5-yl)-3-methoxy-3- methylcyclobutyl)- phenyl)propanoic acid | (400 MHz, CD₃OD) δ 7.66 (d, J = 10.4 Hz, 1H), 7.55 (dd, J = 3.2, 6.0 Hz, 1H), 7.53 (d, J = 3.2 Hz, 1H), 7.21-7.16 (m, 2H), 7.07 (s, 1H), 7.02 (s, 1H), 6.93 (dt, J = 3.6, 8.8 Hz, 1H), 6.87 (dt, J = 2.0, 10.0 Hz, 1H), 6.77 (d, J = 9.6 Hz, 1H), 3.33 (s, 3H), 3.18 (s, 3H), 2.89 (t, J = 7.6 Hz, 2H), 2.84 (d, J = 12.8 Hz, 2H), 2.77 (d, J = 12.8 Hz, 2H), 2.58 (t, J = 7.6 Hz, 2H), 1.27 (s, 3H) ppm. | 0.86 | 654 [M + H]⁺ |
| 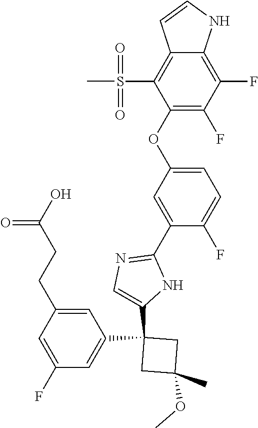 [SMILES: CO[C@]1(C)C[C@@](c2cc(F)cc(CCC(=O)O)c2)(c2cnc(-c3cc(Oc4c(F)c(F)c5[nH]ccc5c4S(C)(=O)=O)ccc3F)[nH]2)C1] | 74 | 3-(3-((1r,3r)-1-(2-(5-((6,7- difluoro-4-(methylsulfonyl)- 1H-indol-5-yl)oxy)-2- fluorophenyl)-1H-imidazol- 5-yl)-3-methoxy-3- methylcyclobutyl)-5- fluorophenyl)propanoic acid | (400 MHz, CD₃OD) δ 7.47 (d, J = 3.2 Hz, 1H), 7.46-7.44 (m, 1H), 7.15 (t, J = 3.2 Hz, 1H), 7.12-7.10 (m, 1H), 6.96 (s, 1H), 6.93-6.89 (m, 1H), 6.84-6.82 (m, 1H), 6.77-6.75 (m, 1H), 6.26 (s,1H), 3.20 (s, 3H), 3.16 (s, 3H), 2.85-2.80 (m, 4H), 2.61 (d, J = 13.2 Hz, 2H), 2.50 (t, J = 7.6 Hz, 2H), 1.12 (s, 3H) ppm. | 0.95 | 672 [M + H]⁺ |
| 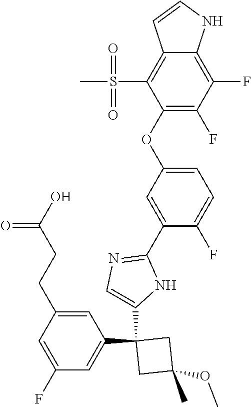 [SMILES: CO[C@]1(C)C[C@](c2cc(F)cc(CCC(=O)O)c2)(c2cnc(-c3cc(Oc4c(F)c(F)c5[nH]ccc5c4S(C)(=O)=O)ccc3F)[nH]2)C1] | 75 | 3-(3-((1s,3s)-1-(2-(5-((6,7- difluoro-4-(methylsulfonyl)- 1H-indol-5-yl)oxy)-2- fluorophenyl)-1H-imidazol- 5-yl)-3-methoxy-3- methylcyclobutyl)-5- fluorophenyl)propanoic acid | (400 MHz, CD₃OD) δ 7.58 (d, J = 3.2 Hz, 1H), 7.58-7.56 (m, 1H), 7.27 (t, J = 3.2 Hz, 1H), 7.24-7.19 (m, 1H), 7.08 (s, 1H), 7.02-6.98 (m, 2H), 6.87 (d, J = 10.0 Hz, 1H), 6.77 (d, J = 9.6 Hz, 1H), 3.33 (s, 3H), 3.18 (s, 3H), 2.90 (t, J = 7.6 Hz, 2H), 2.87-2.74 (m, 4H), 2.58 (t, J = 7.6 Hz, 2H), 1.27 (s, 3H) ppm.. | — | 654 [M + H]⁺ |

### Example 76

### 3-(3-(1-Cyano-2-(2-(2-fluoro-5-((6-fluoro-4-(methylsulfonyl)-1H-indol-5-yl)oxy)phenyl)-1H-imidazol-5-yl)propan-2-yl)phenyl)propanoic acid

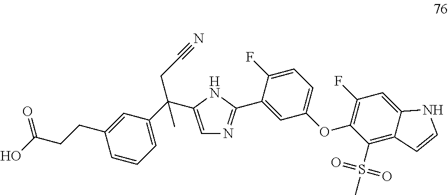 [SMILES: CC(CC#N)(c1cccc(CCC(=O)O)c1)c1cnc(-c2cc(Oc3c(F)cc4[nH]ccc4c3S(C)(=O)=O)ccc2F)[nH]1]

### A. 3-Cyano-2-(3-iodophenyl)-2-methylpropanoic acid

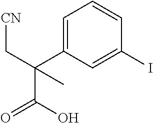

To a stirred and cooled (−78° C.) solution of 2-(3-iodophenyl)propanoic acid (5.18 g, 18.7 mmol) in THF (60 mL) was added, dropwise over 10 minutes, a 2.0 M solution of LDA in THF/heptane/ethylbenzene (18.7 mL, 37.4 mmol). The reaction was stirred at −78° C. for 30 minutes before adding, dropwise over 4-5 minutes, 2-bromoacetonitrile (2.6 mL, 37.3 mmol). After another two hours at −78° C., the reaction was allowed to slowly warm to room temperature and continued overnight. After this time, the reaction was quenched and then acidified (to ˜pH 3) with the addition of 1.0 N hydrochloric acid. The reaction was then partitioned between water (100 mL) and ethyl acetate (80 mL). The organic layer was combined with additional extracts (ethyl acetate, 2×80 mL), dried over sodium sulfate and concentrated. The residue was purified by flash chromatography over silica (10-50% ethyl acetate in petroleum ether) to afford the title compound as a pale amber solid (3.42 g, 58%). MS: 316 m/z [M+H]⁺.

### B. 5-Bromo-3-(3-iodophenyl)-3-methyl-4-oxopentanenitrile

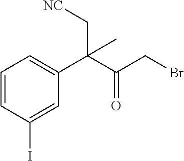 [SMILES: CC(CC#N)(C(=O)CBr)c1cccc(I)c1]

Step A product (2.00 g, 6.35 mmol) was taken up in thionyl chloride (10 mL) and stirred at room temperature for two hours. The mixture was then concentrated and the residue was co-evaporated with heptane (30 mL) several times to remove trace thionyl chloride. To a stirred solution of this crude acid chloride in 1:1 acetonitrile/THF (20 mL) was added a 2.0 M solution of trimethylsilyldiazomethane in hexanes (9.50 mL, 19.0 mmol). The mixture was stirred overnight at room temperature and then concentrated to afford the crude α-diazoketone intermediate. This material was dissolved in acetic acid (10 mL). To this stirred and cooled (0° C.) solution was added a 33% solution of hydrogen bromide in acetic acid (2.0 mL, 11.0 mmol; vigorous gas evolution observed). The reaction was allowed to warm to room temperature and stirring was continued overnight. After this time, the mixture was concentrated and the residue was partitioned between ethyl acetate (100 mL) and a saturated aqueous sodium bicarbonate solution (100 mL). The organic layer was washed with brine (1×100 mL), dried over sodium sulfate and concentrated.

The crude product was purified by flash chromatography over silica (15-35% ethyl acetate in petroleum ether) to afford the title compound as a light brown solid (0.582 g, 23% over three steps). MS: 392, 394 m/z [M+H]⁺.

### C. 3-(2-(2-Fluoro-5-((6-fluoro-4-(methylthio)-1H-indol-5-yl)oxy)phenyl)-1H-imidazol-5-yl)-3-(3-iodophenyl)butanenitrile

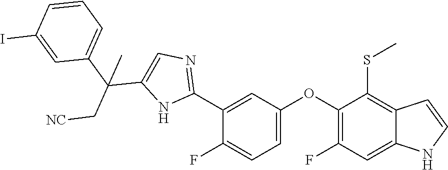 [SMILES: CSc1c(Oc2ccc(F)c(-c3ncc(C(C)(CC#N)c4cccc(I)c4)[nH]3)c2)c(F)cc2[nH]ccc12]

To a stirred solution of 2-fluoro-5-((6-fluoro-4-(methylthio)-1H-indol-5-yl)oxy)benzimidamide (0.305 g, 0.916 mmol, Intermediate 24-4) and step B product (0.395 g, 1.01 mmol) in DMF (10 mL) was added potassium carbonate (0.253 g, 1.83 mmol). The reaction was heated at 80° C. for four hours and then cooled to room temperature and diluted with water (20 mL). The resulting suspension was extracted with ethyl acetate (3×30 mL) and the combined extracts were dried over sodium sulfate and concentrated. The residue was purified by flash chromatography over silica (15-35% ethyl acetate in petroleum ether) to afford the title compound as a pale amber solid (0.460 g, 85%). MS: 627 m/z [M+H]⁺.

### D. Ethyl (E)-3-(3-(1-cyano-2-(2-(2-fluoro-5-((6-fluoro-4-(methylthio)-1H-indol-5-yl)oxy)phenyl)-1H-imidazol-5-yl)propan-2-yl)phenyl)acrylate

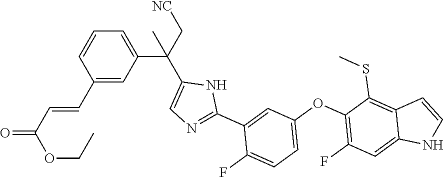 [SMILES: CCOC(=O)/C=C/c1cccc(C(C)(CC#N)c2cnc(-c3cc(Oc4c(F)cc5[nH]ccc5c4SC)ccc3F)[nH]2)c1]

To a stirred solution of step C product (0.456 g, 0.728 mmol) in dioxane (15 mL was added tri(o-tolyl)phosphine (0.067 mg, 0.220 mmol), ethyl acrylate (0.220 g, 2.20 mmol) and Pd(OAc)₂ (16.0 mg, 0.071 mmol). The reaction mixture was heated overnight at 100° C., cooled to room temperature and diluted with water (20 mL). The suspension was extracted with ethyl acetate (3×30 mL) and the combined extracts were dried over sodium sulfate and concentrated. The residue was purified by flash chromatography over silica (15-35% ethyl acetate in petroleum ether) to afford the title compound as an amber solid (0.305 mg, 70%). MS: 599 m/z [M+H]⁺.

### E. Ethyl 3-(3-(1-cyano-2-(2-(2-fluoro-5-((6-fluoro-4-(methylthio)-1H-indol-5-yl)oxy)phenyl)-1H-imidazol-5-yl)propan-2-yl)phenyl)propanoate

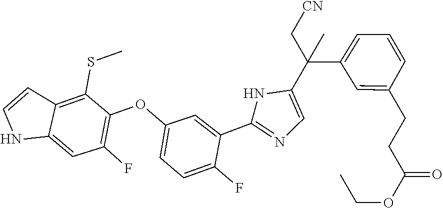 [SMILES: CCOC(=O)CCc1cccc(C(C)(CC#N)c2cnc(-c3cc(Oc4c(F)cc5[nH]ccc5c4SC)ccc3F)[nH]2)c1]

A stirred suspension of step D product (0.305 g, 0.509 mmol) and 10% Pd/C (0.080 g) in ethanol (10 mL) was cycled between vacuum and a nitrogen atmosphere three times. The reaction vessel was evacuated a final time and backfilled with hydrogen (via balloon). The reaction was stirred overnight at room temperature and then filtered through a pad of Celite, which was subsequently rinsed with additional ethanol (total, ˜90 mL). The combined filtrate was concentrated and the residue was purified by reversed phase, preparative HPLC to afford the title compound as an off-white solid (0.190 g, 62%). MS: 601 m/z [M+H]⁺.

### F. Ethyl 3-(3-(1-cyano-2-(2-(2-fluoro-5-((6-fluoro-4-(methylsulfonyl)-1H-indol-5-yl)oxy)phenyl)-1H-imidazol-5-yl)propan-2-yl)phenyl)propanoate

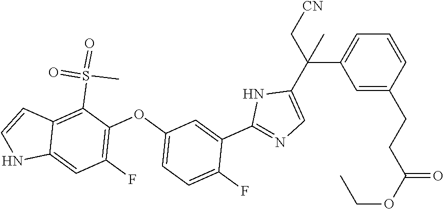 [SMILES: CCOC(=O)CCc1cccc(C(C)(CC#N)c2cnc(-c3cc(Oc4c(F)cc5[nH]ccc5c4S(C)(=O)=O)ccc3F)[nH]2)c1]

To a stirred solution of step E product (0.190 g, 0.317 mmol) in 1:1 methanol/THF (14 mL) was added a solution of ammonium molybdate tetrahydrate (0.280 g, 0.226 mmol) in 30% aqueous hydrogen peroxide (1.4 mL). The reaction was stirred at room temperature for three hours and then partitioned between ethyl acetate (70 mL) and water (30 mL). The organic layer was washed with additional water (2×30 mL) and brine (2×30 mL), dried over sodium sulfate and concentrated. The residue was purified by flash chromatography over silica (0-60% ethyl acetate in petroleum ether) to afford the title compound as a white solid (98.0 mg, 49%).

### G. Example 76, 3-(3-(1-Cyano-2-(2-(2-fluoro-5-((6-fluoro-4-(methylsulfonyl)-1H-indol-5-yl)oxy)phenyl)-1H-imidazol-5-yl)propan-2-yl)phenyl)propanoic acid

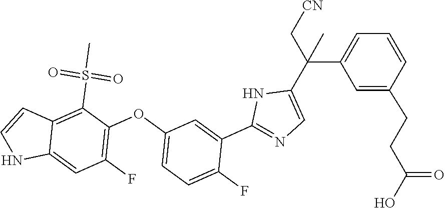 [SMILES: CC(CC#N)(c1cccc(CCC(=O)O)c1)c1cnc(-c2cc(Oc3c(F)cc4[nH]ccc4c3S(C)(=O)=O)ccc2F)[nH]1]

To a stirred solution of step F product (0.100 g, 0.158 mmol) in THF (10 mL) was added 1 M lithium hydroxide (1.6 mL, 1.6 mmol). The reaction was stirred overnight at room temperature and then concentrated. The residue was taken up in water (2 mL). This stirred solution was acidified (˜pH 4) with the dropwise addition of 1.0 N HCl. The precipitate which formed was collected by suction filtration and the purified by reversed phase, preparative HPLC to afford the title compound as a white solid (53.0 mg, 56%). ¹H NMR (400 MHz, DMSO-d₆) δ 7.68 (d, J=10.4 Hz, 1H), 7.59 (s, 1H), 7.55 (d, J=3.2 Hz, 1H), 7.46 (dd, J=5.6, 3.2 Hz, 1H), 7.35 (t, J=9.6 Hz, 1H), 7.32 (t, J=7.6 Hz, 1H), 7.22-7.16 (m, 5H), 3.38-3.33 (m, 5H), 2.91 (t, J=7.6 Hz, 2H), 2.58 (t, J=7.6 Hz, 2H), 1.92 (s, 3H) ppm. MS: 605 m/z [M+H]⁺.

### Example 77

### 3-(3-(1-cyano-2-(2-(5-((4,6-difluoro-1H-indol-5-yl)oxy)-2-fluorophenyl)-1H-imidazol-5-yl)propan-2-yl)phenyl)propanoic acid

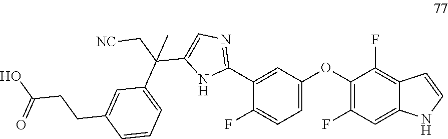 [SMILES: CC(CC#N)(c1cccc(CCC(=O)O)c1)c1cnc(-c2cc(Oc3c(F)cc4[nH]ccc4c3F)ccc2F)[nH]1]

Exchanging 2-fluoro-5-((6-fluoro-4-(methylthio)-1H-indol-5-yl)oxy)benzimidamide (Intermediate 24-4) for 5-((4,6-difluoro-1H-indol-5-yl)oxy)-2-fluorobenzimidamide (intermediate 24-9) in step C, procedures analogous to those described in steps C-G of the Example 76 synthesis were used to prepare the title compound as a white solid. ¹H NMR (400 MHz, CD₃OD) δ 7.52-7.48 (m, 1H), 7.31 (d, J=2.8 Hz, 1H), 7.25-7.19 (m, 3H), 7.17-7.11 (m, 3H), 7.05 (s, 1H), 6.97-6.93 (m, 1H), 6.56 (d, J=2.8 Hz, 1H), 3.30 (s, 2H), 2.88 (t, J=8.0 Hz, 2H), 2.53 (t, J=8.0 Hz, 2H), 1.85 (s, 3H) ppm. MS: 545 m/z [M+H]⁺.

### Example 78

### 3-(3-(3,3-Difluoro-1-(2-(2-fluoro-5-((6-fluoro-4-(methylsulfonyl)-1H-indol-5-yl)oxy)phenyl)-1H-imidazol-5-yl)cyclobutyl)phenyl)propanoic acid

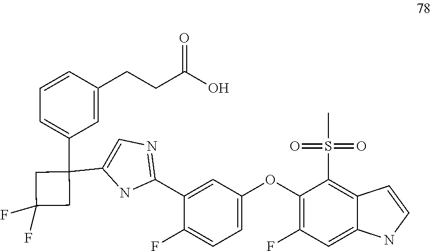

### A. 1-(3-Bromophenyl)-3,3-difluorocyclobutane-1-carbonitrile

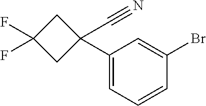 [SMILES: N#CC1(c2cccc(Br)c2)CC(F)(F)C1]

To a stirred and cooled (0° C.) solution of 1-(3-bromophenyl)-3-oxocyclobutane-1-carbonitrile (Step B product from the syntheses of Example 70; 1.80 g, 7.20 mmol) in dichloromethane (40 mL) was added, portionwise over 4-5 minutes, DAST (3.58 g, 21.6 mmol). The reaction was stirred overnight at room temperature and then partitioned between dichloromethane (150 mL), and water (50 mL). The organic layer was washed with a saturated aqueous sodium carbonate solution (1×30 mL) and brine (1×50 mL), dried over sodium sulfate and concentrated. The residue was purified by flash chromatography over silica (0-10% ethyl acetate in petroleum ether) to afford the title compound as an oil (1.00 g, 51%). ¹H NMR (400 MHz, CDCl₃) δ 7.60 (s, 1H), 7.53 (d, J=7.6 Hz, 1H), 7.40 (d, J=7.6 Hz, 1H), 7.33 (t, J=7.6 Hz, 1H), 3.56-3.47 (m, 2H), 3.20 (q, J=13.6 Hz, 2H) ppm.

### B. 1-(3-Bromophenyl)-3,3-difluorocyclobutane-1-carboxylic acid

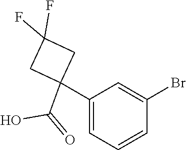 [SMILES: O=C(O)C1(c2cccc(Br)c2)CC(F)(F)C1]

To a stirred solution of step A product (0.980 g, 3.60 mmol) in 5:1 methanol/water (30 mL) was added potassium hydroxide (4.00 g, 71.3 mmol). The reaction was heated overnight at 80° C. and then concentrated to remove the organic solvent. The remaining aqueous solution was acidified, with stirring, to ˜pH 2 with the dropwise addition of 1.0 N hydrochloric acid. The resulting suspension was diluted with ethyl acetate (200 mL). This solution was washed with water (2×50 mL) and brine (1×50 mL), dried over sodium sulfate and concentrated. Crude title compound was afforded as white solid (0.891 g, 85%). ¹H NMR (400 MHz, CDCl₃) δ 7.40-7.37 (m, 2H), 7.19-7.17 (m, 2H), 3.45-3.36 (m, 2H), 2.98 (m, 2H) ppm.

### C. 3,3-Difluoro-1-(3-(3-methoxy-3-oxoprop-1-en-1-yl)phenyl)cyclobutane-1-carboxylic acid

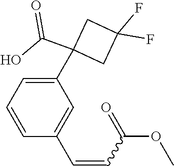 [SMILES: COC(=O)C=Cc1cccc(C2(C(=O)O)CC(F)(F)C2)c1]

To a stirred solution of step B product (0.600 g, 2.06 mmol) in DMF (25 mL) was added ethyl acrylate (440 μL, 4.03 mmol) and triethylamine (1.4 mL, 10 mmol) and tri(o-tolyl)phosphine (0.122 g, 0.400 mmol) and palladium(II) acetate (45.0 mg, 0.200 mmol). The reaction was heated overnight at 110° C., cooled to room temperature and diluted with ethyl acetate (100 mL). This solution was washed with brine (2×30 mL), dried over sodium sulfate and concentrated. The residue was purified by flash chromatography over silica (0-40% ethyl acetate in petroleum ether) to afford the title compound as a white solid (0.520 g, 85%). ¹H NMR (400 MHz, CDCl₃) δ 7.68 (d, J=16.0 Hz 1H), 7.48-7.33 (m, 4H), 6.45 (d, J=16.0 Hz, 1H), 3.81 (s, 3H), 3.55-3.46 (m, 2H), 3.08 (q, J=13.6 Hz, 2H) ppm.

### D. 3,3-Difluoro-1-(3-(3-methoxy-3-oxopropyl)phenyl)cyclobutane-1-carboxylic acid

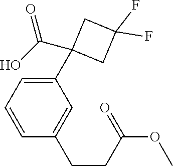 [SMILES: COC(=O)CCc1cccc(C2(C(=O)O)CC(F)(F)C2)c1]

A stirred suspension of step C product (0.520 g, 1.76 mmol) and 10% Pd/C (0.100 g) in methanol (20 mL) was cycled between vacuum and a nitrogen atmosphere three times. The reaction vessel was evacuated a final time and backfilled with hydrogen (via balloon). The reaction was heated overnight at 38° C. and then cooled to room temperature and filtered through a pad of Celite. The filtering agent was rinsed with additional methanol (total, ˜80 mL) and the combined filtrate was concentrated. Crude title compound was afforded as an oil (0.498 g, 95%). ¹H NMR (400 MHz, CDCl₃) δ 7.30 (t, J=7.6 Hz, 1H), 7.26 (s, 1H), 7.17-7.14 (m, 2H), 3.66 (s, 3H), 3.52-3.43 (m, 2H), 3.10-3.00 (m, 2H), 2.96 (t, J=7.6 Hz, 2H), 2.63 (t, J=7.6 Hz, 2H) ppm.

### E. Methyl 3-(3-(1-(2-bromoacetyl)-3,3-difluorocyclobutyl)phenyl)propanoate

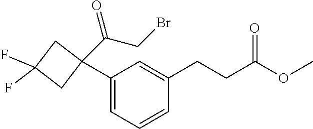 [SMILES: COC(=O)CCc1cccc(C2(C(=O)CBr)CC(F)(F)C2)c1]

To a stirred solution of 3,3-difluoro-1-(3-(3-methoxy-3-oxopropyl)phenyl)cyclobutane-1-carboxylic acid (0.710 g, 2.38 mmol) in dichloromethane (30 mL) was added oxalyl chloride (700 μL, 8.16 mmol) followed by three drops of DMF. After three hours at room temperature, the reaction was concentrated. The residue was co-evaporated several times with heptane to remove trace oxalyl chloride. The crude acid chloride was dissolved in 1:1 tetrahydrofuran/acetonitrile (10 mL). To this stirred and cooled (0° C.) solution was added, dropwise over five minutes, a 2.0 M solution of trimethylsilyldiazomethane in hexanes (4.9 mL, 9.8 mmol). The reaction mixture was allowed to warm to room temperature and stirred overnight. After this time, the mixture was returned to 0° C. and treated, dropwise over 3-4 minutes, with a 33% solution of hydrogen bromide in acetic acid (1.90 mL, 10.5 mmol; vigorous gas evolution observed). The reaction was allowed to warm to room temperature and then stirred for another 45 minutes before partitioning between ethyl acetate (150 mL) and water (75 mL). The organic layer was washed additional water (1×75 mL), saturated aqueous sodium bicarbonate solution (1×80 mL) and brine (60 mL). The solution was then dried over sodium sulfate and concentrated to afford a residue which was purified by flash chromatography over silica (0-20% ethyl acetate in petroleum ether). The title compound was afforded as an oil (0.670 g, 75%). ¹H NMR (400 MHz, CDCl₃) δ 7.34 (t, J=7.6 Hz, 1H), 7.18 (d, J=7.6 Hz, 1H), 7.11 (d, J=7.6 Hz, 1H), 7.09 (s, 1H), 3.86 (s, 2H), 3.66 (s, 3H), 3.52-3.43 (m, 2H), 3.09-2.99 (m, 2H), 2.96 (t, J=7.6 Hz, 2H), 2.63 (t, J=7.6 Hz, 2H) ppm.

### F. Methyl 3-(3-(3,3-difluoro-1-(2-(2-fluoro-5-((6-fluoro-4-(methylthio)-1H-indol-5-yl)oxy)phenyl)-1H-imidazol-5-yl)cyclobutyl)phenyl)propanoate

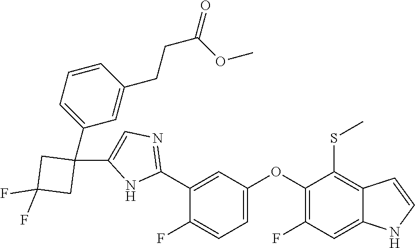 [SMILES: COC(=O)CCc1cccc(C2(c3cnc(-c4cc(Oc5c(F)cc6[nH]ccc6c5SC)ccc4F)[nH]3)CC(F)(F)C2)c1]

To a stirred solution of 2-fluoro-5-((6-fluoro-4-(methylthio)-1H-indol-5-yl)oxy)benzimidamide (Intermediate 24-4; 0.180 g, 0.540 mmol) and step E product (0.206 g, 0.550 mmol) in acetonitrile (3 mL) was added sodium bicarbonate (92.0 mg, 1.10 mmol). The reaction was heated overnight at 75° C., cooled to room temperature and partitioned between water (50 mL) and ethyl acetate (30 mL). The organic layer was combined with a second extract (ethyl acetate, 1×30 mL), washed with brine (1×50 mL) and dried over sodium sulfate. The solution was concentrated to afford a residue which was purified by flash chromatography over silica (0-50% ethyl acetate in petroleum ether) to afford the title compound as an off-white solid (0.190 g, 58%). MS: 610 m/z [M+H]⁺.

### G. Methyl 3-(3-(3,3-difluoro-1-(2-(2-fluoro-5-((6-fluoro-4-(methylsulfonyl)-1H-indol-5-yl)oxy)phenyl)-1H-imidazol-5-yl)cyclobutyl)phenyl)propanoate

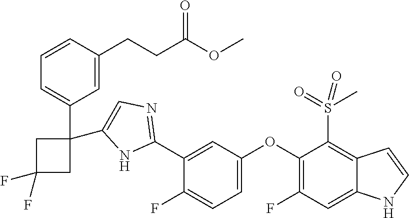 [SMILES: COC(=O)CCc1cccc(C2(c3cnc(-c4cc(Oc5c(F)cc6[nH]ccc6c5S(C)(=O)=O)ccc4F)[nH]3)CC(F)(F)C2)c1]

To a stirred solution of step F product (0.190 g, 0.312 mmol) in methanol (20 mL) was added a mixture of ammonium molybdate tetrahydrate (0.400 g, 0.324 mmol) in 30% aqueous hydrogen peroxide (2 mL). The reaction was stirred at room temperature for two hours and then partitioned between aqueous sodium sulfite solution (50 mL) and ethyl acetate (50 mL). The organic layer was combined with additional extracts (ethyl acetate, 2×50 mL), washed with aqueous sodium sulfite solution and brine, dried over sodium sulfate and concentrated. The residue was purified by flash chromatography over silica (0-30% ethyl acetate in petroleum ether) to afford the title compound as a white solid (0.150 g, 75%). MS: 642 m/z [M+H]⁺.

### H. Example 78, 3-(3-(3,3-Difluoro-1-(2-(2-fluoro-5-((6-fluoro-4-(methylsulfonyl)-1H-indol-5-yl)oxy)phenyl)-1H-imidazol-5-yl)cyclobutyl)phenyl)propanoic acid

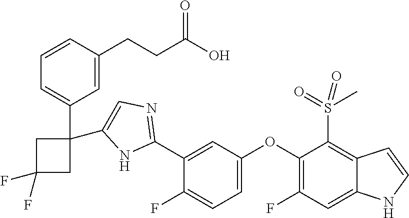 [SMILES: CS(=O)(=O)c1c(Oc2ccc(F)c(-c3ncc(C4(c5cccc(CCC(=O)O)c5)CC(F)(F)C4)[nH]3)c2)c(F)cc2[nH]ccc12]

To a stirred solution of step G product (0.140 g, 0.218 mmol) in methanol 3:1 THF/methanol (8 mL) was added a 1.0 M aqueous solution of lithium hydroxide (1.0 mL, 1.0 mmol). The reaction was stirred at room temperature for two hours and then acidified (˜pH 4) by the dropwise addition of 1.0 N hydrochloric acid. The resulting suspension was extracted with ethyl acetate (3×45 mL). The combined organic extracts were washed with water (2×25 mL) and brine (1×15 mL), dried over sodium sulfate and concentrated afford the title compound as a white solid (64.5 mg, 46%). ¹H NMR (400 MHz, CD₃OD) 7.64 (d, J=10.8 Hz, 1H), 7.57-7.55 (m, 1H), 7.51 (d, J=2.8 Hz, 1H), 7.24-7.14 (m, 5H), 7.10-7.08 (m, 1H), 6.90-6.88 (m, 2H), 3.34 (s, 3H), 3.38-3.20 (m, 4H), 2.90 (t, J=7.6 Hz, 2H), 2.58 (t, J=7.6 Hz, 2H) ppm. MS: 628 m/z [M+H]⁺.

### Example 79

### 3-(3-(3,3-Difluoro-1-(2-(2-fluoro-5-((6-fluoro-4-(methylsulfonyl)-1H-indol-5-yl)oxy)phenyl)thiazol-4-yl)cyclobutyl)phenyl)propanoic acid

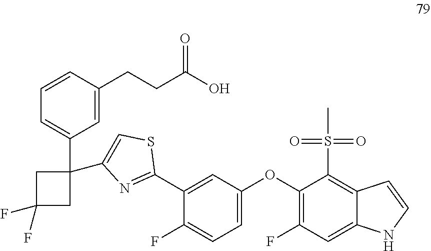 [SMILES: CS(=O)(=O)c1c(Oc2ccc(F)c(-c3nc(C4(c5cccc(CCC(=O)O)c5)CC(F)(F)C4)cs3)c2)c(F)cc2[nH]ccc12]

### A. Methyl 3-(3-(3,3-difluoro-1-(2-(2-fluoro-5-((6-fluoro-4-(methylthio)-1H-indol-5-yl)oxy)phenyl)thiazol-4-yl)cyclobutyl)phenyl)propanoate

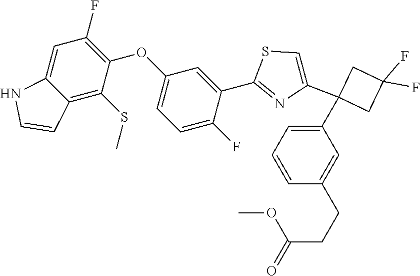 [SMILES: COC(=O)CCc1cccc(C2(c3csc(-c4cc(Oc5c(F)cc6[nH]ccc6c5SC)ccc4F)n3)CC(F)(F)C2)c1]

To a stirred solution of 2-fluoro-5-((6-fluoro-4-(methylthio)-1H-indol-5-yl)oxy)benzothioamide (Intermediate 33-1; 0.200 g, 0.571 mmol) in ethanol (25 mL) was added methyl 3-(3-(1-(2-bromoacetyl)-3,3-difluorocyclobutyl)phenyl)propanoate (step E product from synthesis of Example 78; 0.214 g, 0.570 mmol). The reaction was heated overnight at 80° C., cooled to room temperature and partitioned between ethyl acetate (100 mL) and water (100 mL). The organic layer was washed with brine (1×100 mL), dried over sodium sulfate and concentrated. The residue was purified by flash chromatography over silica (60% ethyl acetate in petroleum ether) to afford the title compound as a yellow oil (0.300 g, 84%). MS: 627 m/z [M+H]⁺.

### B. Example 79, 3-(3-(3,3-Difluoro-1-(2-(2-fluoro-5-((6-fluoro-4-(methylsulfonyl)-1H-indol-5-yl)oxy)phenyl)thiazol-4-yl)cyclobutyl)phenyl)propanoic acid

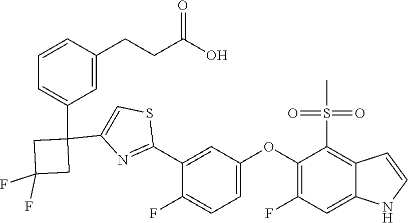 [SMILES: CS(=O)(=O)c1c(Oc2ccc(F)c(-c3nc(C4(c5cccc(CCC(=O)O)c5)CC(F)(F)C4)cs3)c2)c(F)cc2[nH]ccc12]

Exchanging methyl 3-(3-((2-(2-fluoro-5-((6-fluoro-4-(methylthio)-1H-indol-5-yl)oxy)phenyl)-1H-imidazol-5-yl)methyl)phenyl)propanoate for step A product, procedures analogous to those described in steps B and C of the Example 22 synthesis were used to prepare the title compound as a white solid. ¹H NMR (400 MHz, CD₃OD) δ 7.85 (dd, J=5.6 Hz, 3.2 Hz, 1H), 7.70 (d, J=10.4 Hz, 1H), 7.56 (d, J=3.2 Hz, 1H), 7.25-7.12 (m, 5H), 7.14-7.12 (m, 1H), 7.04 (s, 1H), 7.02-6.99 (m, 1H), 3.54-3.44 (m, 2H), 3.36 (s, 3H), 3.32-3.20 (m, 2H), 2.91 (t, J=7.6 Hz, 2H), 2.61 (t, J=7.6 Hz, 2H) ppm. MS: 645 m/z [M+H]⁺.

### Example 80

### 3-(3-(1-(2-(5-((4-(dimethylphosphoryl)-6-fluoro-1H-indol-5-yl)oxy)-2-fluorophenyl)-1H-imidazol-5-yl)-3,3-difluorocyclobutyl)-5-fluorophenyl)propanoic acid

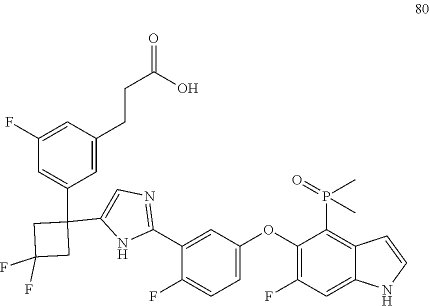 [SMILES: CP(C)(=O)c1c(Oc2ccc(F)c(-c3ncc(C4(c5cc(F)cc(CCC(=O)O)c5)CC(F)(F)C4)[nH]3)c2)c(F)cc2[nH]ccc12]

### A. Methyl 3-(3-(1-(2-bromoacetyl)-3,3-difluorocyclobutyl)-5-fluorophenyl)propanoate

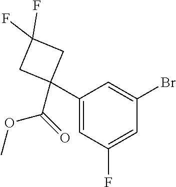 [SMILES: COC(=O)C1(c2cc(F)cc(Br)c2)CC(F)(F)C1]

Exchanging 1-(3-bromophenyl)-3-oxocyclobutane-1-carbonitrile for methyl 1-(3-bromo-5-fluorophenyl)-3-oxocyclobutane-1-carboxylate (Step C product from Example 72 synthesis), the reaction conditions for deoxofluorination described in step A of the Example 78 synthesis were used to prepare the title compound. MS: 333, 335 m/z [M+H]⁺.

### B. 1-(3-Bromo-5-fluorophenyl)-3,3-difluorocyclobutane-1-carboxylic acid

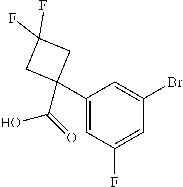 [SMILES: O=C(O)C1(c2cc(F)cc(Br)c2)CC(F)(F)C1]

Exchanging methyl 1-(3-bromo-5-fluorophenyl)-3-oxocyclobutane-1-carboxylate for step A product, the reaction conditions for methyl ester hydrolysis described in step D of the Example 72 synthesis were used to prepare the title compound.

### C. Methyl 3-(3-(1-(2-bromoacetyl)-3,3-difluorocyclobutyl)-5-fluorophenyl)propanoate

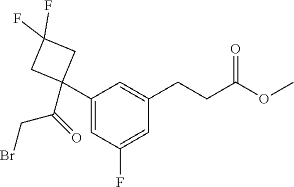 [SMILES: COC(=O)CCc1cc(F)cc(C2(C(=O)CBr)CC(F)(F)C2)c1]

Exchanging 1-(3-bromophenyl)-3,3-difluorocyclobutane-1-carboxylic acid for step B product, procedures analogous to those described in steps C-E of the Example 78 synthesis were used to prepare the title compound as a yellow oil. MS: 393, 395 m/z [M+H]⁺.

### D. Example 80, 3-(3-(1-(2-(5-((4-(Dimethylphosphoryl)-6-fluoro-1H-indol-5-yl)oxy)-2-fluorophenyl)-1H-imidazol-5-yl)-3,3-difluorocyclobutyl)-5-fluorophenyl)propanoic acid

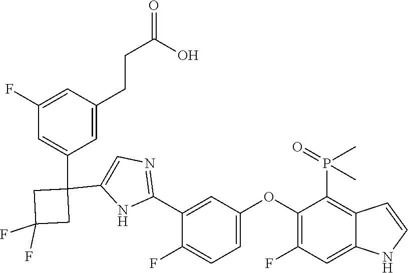 [SMILES: CP(C)(=O)c1c(Oc2ccc(F)c(-c3ncc(C4(c5cc(F)cc(CCC(=O)O)c5)CC(F)(F)C4)[nH]3)c2)c(F)cc2[nH]ccc12]

Exchanging methyl 3-(3-(1-(2-bromoacetyl)-3,3-difluorocyclobutyl)phenyl)propanoate for methyl 3-(3-(1-(2-bromoacetyl)-3,3-difluorocyclobutyl)-5-fluorophenyl)propanoate (0.300 g, 0.763 mmol) and 2-fluoro-5-((6-fluoro-4-(methylthio)-1H-indol-5-yl)oxy)benzimidamide for 5-((4-(dimethylphosphoryl)-6-fluoro-1H-indol-5-yl)oxy)-2-fluorobenzimidamide (Intermediate 24-16; 0.250 g, 0.688 mmol), procedures analogous to those described in steps F and H of the Example 78 synthesis (imidazole formation via condensation of an amidine and α-bromoketone followed by hydrolysis of a 3-phenylpropanoate ester to a carboxylic acid) were used to prepare the title compound as a white solid (0.190 g, 39% over two steps). ¹H NMR (400 MHz, CD₃OD) δ 7.56-7.53 (m, 2H), 7.47 (d, J=3.2 Hz, 1H), 7.29-7.24 (m, 2H), 7.15 (s, 1H), 7.09 (s, 1H), 7.01-6.96 (m, 2H), 6.89 (d, J=9.2 Hz, 1H), 3.44-3.34 (m, 2H), 3.32-3.21 (m, 2H), 2.92 (t, J=7.6 Hz, 2H), 2.61 (t, J=7.6 Hz, 2H), 1.87 (s, 3H), 1.82 (s, 3H) ppm. MS: 644.2 m/z [M+H]⁺.

### Example 81

### 3-(3-(1-(2-(2-Fluoro-5-((6-fluoro-4-(methylsulfonyl)-1H-indol-5-yl)oxy)phenyl)oxazol-4-yl)cyclopropyl)phenyl)propanoic acid

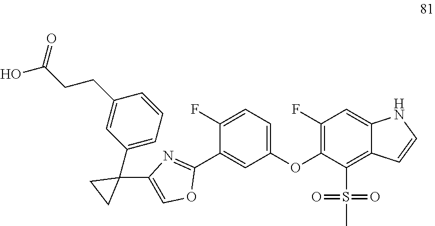 [SMILES: CS(=O)(=O)c1c(Oc2ccc(F)c(-c3nc(C4(c5cccc(CCC(=O)O)c5)CC4)co3)c2)c(F)cc2[nH]ccc12]

### A. 2-Fluoro-5-((6-fluoro-4-(methylthio)-1H-indol-5-yl)oxy)benzoic acid

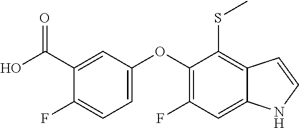 [SMILES: CSc1c(Oc2ccc(F)c(C(=O)O)c2)c(F)cc2[nH]ccc12]

To a stirred suspension of 2-fluoro-5-((6-fluoro-4-(methylthio)-1H-indol-5-yl)oxy)benzonitrile (Intermediate 23; 1.00 g, 3.16 mmol) in ethanol (5 mL) was added a 4.0 N aqueous solution of potassium hydroxide (5.0 mL, 20 mmol). The reaction was heated at 80° C. for twelve hours and then cooled and concentrated. The residue was dissolved in water (˜50 mL) and this stirred solution was acidified (pH 2-3) by the slow addition of 4.0 N hydrochloric acid. The resulting suspension was extracted with ethyl acetate (3×50 mL) and the combined organic extracts were washed with brine (1×50 mL), dried over magnesium sulfate and concentrated. Crude title compound was afforded as a white solid (0.954 g, 90%). MS: 336 m/z [M+H]⁺.

### B. 2-(1-(3-(3-Methoxy-3-oxopropyl)phenyl)cyclopropyl)-2-oxoethyl 2-fluoro-5-((6-fluoro-4-(methylthio)-1H-indol-5-yl)oxy)benzoate

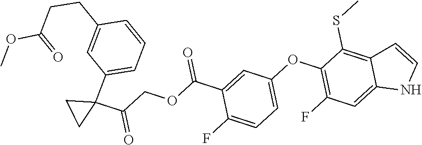 [SMILES: COC(=O)CCc1cccc(C2(C(=O)COC(=O)c3cc(Oc4c(F)cc5[nH]ccc5c4SC)ccc3F)CC2)c1]

To a stirred suspension of step A product (0.550 g, 1.64 mmol) in acetonitrile (20 mL) was added methyl 3-(3-(1-(2-bromoacetyl)cyclopropyl)phenyl)propanoate (Intermediate 21-5; 0.550 g, 1.69 mmol) and triethylamine (418 μL, 3.00 mmol). The reaction was stirred overnight at room temperature and then concentrated. The residue was purified by flash chromatography on silica (50% ethyl acetate in petroleum ether) to afford the title compound as a light yellow oil (0.690 g, 73%). MS: 580 m/z [M+H]⁺.

### C. Methyl 3-(3-(1-(2-(2-fluoro-5-((6-fluoro-4-(methylthio)-1H-indol-5-yl)oxy)phenyl)-oxazol-4-yl)cyclopropyl)phenyl)propanoate

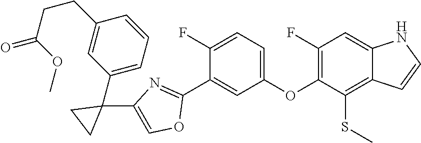 [SMILES: COC(=O)CCc1cccc(C2(c3coc(-c4cc(Oc5c(F)cc6[nH]ccc6c5SC)ccc4F)n3)CC2)c1]

To a stirred suspension of step B product (0.690 g, 1.19 mmol) in acetonitrile (10 mL) was added ammonium acetate (2.40 g, 31.1 mmol). The reaction was heated for twelve hours at 120° C. and then cooled to room temperature and concentrated. The residue was subjected to flash chromatography over silica (50% ethyl acetate in petroleum ether). The title compound was obtained as a yellow solid (0.180 g, to provide methyl 3-(3-(1-(2-(2-fluoro-5-((6-fluoro-4-(methylthio)-1H-indol-5-yl)oxy)phenyl)oxazol-4-yl)cyclopropyl)phenyl)propanoate as a yellow solid (0.180 g, 27%). MS: 561 m/z [M+H]⁺.

### D. Example 81, 3-(3-(1-(2-(2-Fluoro-5-((6-fluoro-4-(methylsulfonyl)-1H-indol-5-yl)oxy)phenyl)oxazol-4-yl)cyclopropyl)phenyl)propanoic acid

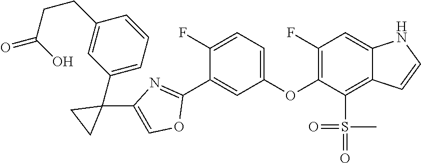 [SMILES: CS(=O)(=O)c1c(Oc2ccc(F)c(-c3nc(C4(c5cccc(CCC(=O)O)c5)CC4)co3)c2)c(F)cc2[nH]ccc12]

Exchanging methyl 3-(3-((2-(2-fluoro-5-((6-fluoro-4-(methylthio)-1H-indol-5-yl)oxy)phenyl)-1H-imidazol-5-yl)methyl)phenyl)propanoate for step C product, procedures analogous to those described in steps B and C of the Example 82 synthesis were used to prepare the title compound as a white solid. ¹H NMR (400 MHz, CD₃OD) δ 7.68 (d, J=10.8 Hz, 1H), 7.54 (d, J=3.2 Hz 1H), 7.51 (dd, J=5.6 Hz, 3.2 Hz, 1H), 7.30-7.21 (m, 6H), 7.14 (d, J=6.8 Hz, 1H), 7.08-7.06 (m, 1H), 3.34 (s, 3H), 2.92 (t, J=7.6 Hz, 2H), 2.61 (t, J=7.6 Hz, 2H), 1.46-1.42 (m, 2H), 1.24-1.22 (m, 2H) ppm. MS: 579 m/z [M+H]⁺.

### Example 82

### 3-(3-(4-(2-(2-Fluoro-5-((6-fluoro-4-((trifluoromethyl)thio)-1H-indol-5-yl)oxy)phenyl)-1H-imidazol-5-yl)tetrahydro-2H-pyran-4-yl)phenyl)propanoic acid

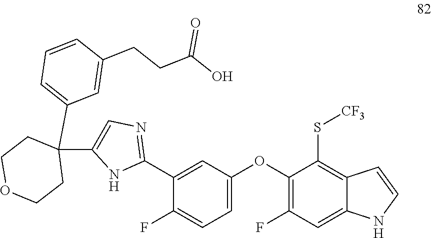 [SMILES: O=C(O)CCc1cccc(C2(c3cnc(-c4cc(Oc5c(F)cc6[nH]ccc6c5SC(F)(F)F)ccc4F)[nH]3)CCOCC2)c1]

### A. 2-Fluoro-5-((6-fluoro-1-tosyl-4-((trifluoromethyl)thio)-1H-indol-5-yl)oxy)benzonitrile

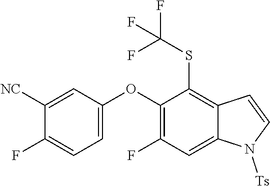 [SMILES: Cc1ccc(S(=O)(=O)n2ccc3c(SC(F)(F)F)c(Oc4ccc(F)c(C#N)c4)c(F)cc32)cc1]

To a stirred and cooled (0° C.) solution of 2-fluoro-5-((6-fluoro-4-mercapto-1-tosyl-1H-indol-5-yl)oxy)benzonitrile (Intermediate 23B; 1.00 g, 2.19 mmol) in DMF (20 mL) was added a 60% dispersion of sodium hydride in mineral oil (0.100 g, 2.50 mmol). The frothy mixture was stirred at 0° C. for 30 minutes and then treated with, in one portion, 5-(trifluoromethyl)dibenzothiophenium trifluoromethanesulfonate (1.00 g, 2.49 mmol). The reaction was allowed to warm to room temperature and stirred overnight. After this time, the reaction was diluted with water (200 mL) and the resulting suspension was extracted with ethyl acetate (3×50 mL). The combined extracts were washed with brine (3×50 mL), dried over sodium sulfate and concentrated. The residue was purified by flash chromatography over silica (0-25% ethyl acetate in petroleum ether) to afford the title compound as a yellow solid (0.360 g, 20%). MS: 547 m/z [M+Na]⁺.

### B. 2-Fluoro-5-((6-fluoro-4-((trifluoromethyl)thio)-1H-indol-5-yl)oxy)benzonitrile

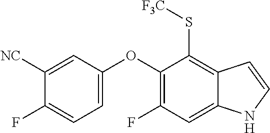 [SMILES: N#Cc1cc(Oc2c(F)cc3[nH]ccc3c2SC(F)(F)F)ccc1F]

To a stirred 1.0 M solution of tetrabutylammonium fluoride in THF (2.8 mL, 2.8 mmol) was added step A product (0.300 g, 0.572 mmol). The reaction mixture was heated at 75° C. for two hours and then partitioned between with water (50 mL) and ethyl acetate (30 mL). The organic layer was combined with additional extracts (ethyl acetate, 2×30 mL), washed with water (1×50 mL) and brine (1×50 mL), dried over sodium sulfate and concentrated. The residue was purified by flash chromatography over silica gel (0-20% ethyl acetate in petroleum ether) to afford the title compound as a white solid (0.160 g, 76%). ¹H NMR (400 MHz, CDCl₃) δ 8.48 (br s, 1H), 7.45 (d, J=10.0 Hz, 1H), 7.41 (t, J=2.8 Hz, 1H), 7.22-7.13 (m, 2H), 6.99 (dd, J=4.4, 2.8 Hz, 1H), 6.84 (dd, J=2.8, 0.4 Hz, 1H) ppm. MS: 371 m/z [M+H]⁺.

### C. 2-Fluoro-5-((6-fluoro-4-((trifluoromethyl)thio)-1H-indol-5-yl)oxy)benzimidamide

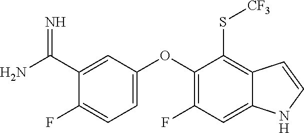 [SMILES: N=C(N)c1cc(Oc2c(F)cc3[nH]ccc3c2SC(F)(F)F)ccc1F]

To a stirred and cooled (0° C.) solution of step B product (160 mg, 0.432 mmol) in anhydrous THF (4.3 mL) was added a 1.0 M solution of lithium bis(trimethylsilyl)amide in THF (4.3 mL, 4.3 mmol). The reaction was allowed to warm to room temperature and stirred overnight. After this time, the mixture as partitioned between water (50 mL) and ethyl acetate (30 mL). The organic layer was combined with additional extracts (ethyl acetate, 2×30 mL), dried over sodium sulfate and concentrated to afford the title compound as a yellow solid (0.160 g, 96%). MS: 388 m/z [M+H]⁺.

### D. Ethyl 3-(3-(4-(2-(2-fluoro-5-((6-fluoro-4-((trifluoromethyl)thio)-1H-indol-5-yl)oxy)phenyl)-1H-imidazol-5-yl)tetrahydro-2H-pyran-4-yl)phenyl)propanoate

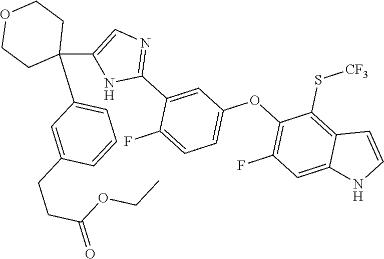 [SMILES: CCOC(=O)CCc1cccc(C2(c3cnc(-c4cc(Oc5c(F)cc6[nH]ccc6c5SC(F)(F)F)ccc4F)[nH]3)CCOCC2)c1]

To a stirred solution of step C product (0.160 g, 0.413 mmol) in DMF (5 mL) was added ethyl 3-(3-(4-(2-bromoacetyl)tetrahydro-2H-pyran-4-yl)phenyl)propanoate (Intermediate 21-4; 0.158 g, 0.412 mmol) and potassium carbonate (57.0 mg, 0.412 mmol). The reaction was heated at 80° C. for two hours and then cooled to room temperature and partitioned between water (50 mL) and ethyl acetate (30 mL). The organic layer was combined with additional extracts (ethyl acetate, 2×30 mL), washed with water (1×50 mL) and brine (1×50 mL) and dried over sodium sulfate. The solution was concentrated and the obtained residue was subjected to flash chromatography over silica (0-20% ethyl acetate in petroleum ether). The title compound was obtained as yellow (80.2 mg, 29%). MS: 672 m/z [M+H]⁺.

### E. Example 82, 3-(3-(4-(2-(2-Fluoro-5-((6-fluoro-4-((trifluoromethyl)thio)-1H-indol-5-yl)oxy)phenyl)-1H-imidazol-5-yl)tetrahydro-2H-pyran-4-yl)phenyl)propanoic acid

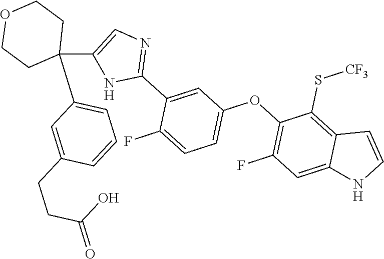 [SMILES: O=C(O)CCc1cccc(C2(c3cnc(-c4cc(Oc5c(F)cc6[nH]ccc6c5SC(F)(F)F)ccc4F)[nH]3)CCOCC2)c1]

To a stirred solution of step D product (80.2 mg, 0.119 mmol) in 3:1 THF/methanol (4 mL) was added a 1.0 M aqueous solution of lithium hydroxide (1.0 mL, 1.0 mmol). The reaction was stirred at room temperature for one hour and then acidified to ˜pH 4 by the dropwise addition of 1.0 N hydrochloric acid. The resulting suspension was extracted with ethyl acetate (3×30 mL) and the combined extracts were washed with brine (1×50 mL), dried over sodium sulfate and concentrated. The residue was purified by flash chromatography over silica (0-80% ethyl acetate in petroleum ether) to afford the title compound as a white solid (40.2 mg, 52%). MS: 644 m/z [M+H]⁺. ¹H NMR (400 MHz, CD₃OD) δ 7.56 (d, J=10.0 Hz, 1H), 7.49 (d, J=3.2 Hz, 1H), 7.40-7.38 (m, 1H), 7.23-7.15 (m, 4H), 7.05 (d, J=7.2 Hz, 1H), 7.00 (s, 1H), 6.87-6.83 (m 1H), 6.72 (d, J=3.2 Hz, 1H), 3.81-3.77 (m, 2H), 3.73-3.70 (m, 2H), 2.88 (t, J=7.6 Hz, 2H), 2.56 (t, J=7.6 Hz, 2H), 2.50-2.47 (m, 2H), 2.35-2.28 (m, 2H) ppm.

### Example 83

### Enantiomer 1 of 5-((2-(2-Fluoro-5-((6-fluoro-4-(methylsulfonyl)-1H-indol-5-yl)oxy)phenyl)-1H-imidazol-5-yl)methyl)-2,3-dihydro-1H-indene-2-carboxylic acid 83A

### And

### Enantiomer 2 of 5-((2-(2-Fluoro-5-((6-fluoro-4-(methylsulfonyl)-1H-indol-5-yl)oxy)phenyl)-1H-imidazol-5-yl)methyl)-2,3-dihydro-1H-indene-2-carboxylic acid 83B

### Absolute Configurations Unknown

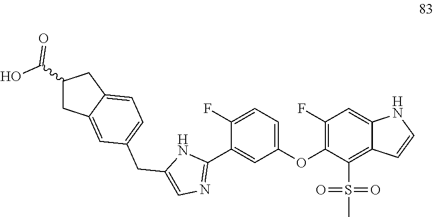 [SMILES: CS(=O)(=O)c1c(Oc2ccc(F)c(-c3ncc(Cc4ccc5c(c4)CC(C(=O)O)C5)[nH]3)c2)c(F)cc2[nH]ccc12]

### A. Methyl 5-bromo-1-oxo-2,3-dihydro-1H-indene-2-carboxylate

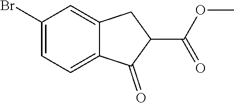

To a stirred and cooled (0° C.) solution of 5-bromo-2,3-dihydro-1H-inden-1-one (5.00 g, 23.7 mmol) in THF (50 mL) was added a 1.0 M solution of lithium bis(trimethylsilyl)amide in THF (59.3 mL, 59.3 mmol). The reaction was allowed to warm to room temperature, stirred for 30 minutes and then returned to 0° C. A solution of methyl chloroformate (2.7 mL g, 35 mmol) in THF (5 mL) was added, dropwise over five minutes, and, again, the reaction was again warmed to room temperature. After two hours, the mixture was partitioned between an saturated aqueous sodium bicarbonate solution (50 mL) and ethyl acetate (40 mL). The organic layer was combined with additional extracts (ethyl acetate, 2×40 mL), washed with brine (1×20 mL) and dried over sodium sulfate. The solution was concentrated and the residue was purified by recrystallization from ethyl acetate (30 mL) to afford the title compound as a yellow solid (2.70 g, 42%). MS: 269, 271 m/z [M+H]⁺.

### B. Methyl 5-bromo-2,3-dihydro-1H-indene-2-carboxylate

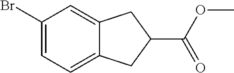

To a stirred and cooled (0° C.) solution of step A product (2.20 g, 8.18 mmol) in trifluoroacetic acid (50 mL) was added triethylsilane (7.84 mL g, 49.1 mmol). The reaction was warmed to room temperature and stirred overnight. After this time, the mixture was concentrate and the residue was dissolved in ethyl acetate (100 mL). This solution was washed with water (3×30 mL) and saturated aqueous sodium bicarbonate solution (2×30 mL), dried over sodium sulfate and concentrated. The residue was purified by flash chromatography over silica (10% ethyl acetate in petroleum ether) to afford the title compound as a yellow solid (1.23 g, 59%). MS: 255, 257 m/z [M+H]⁺.

### C. Methyl 5-allyl-2,3-dihydro-1H-indene-2-carboxylate

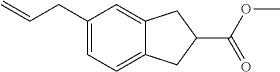 [SMILES: C=CCc1ccc2c(c1)CC(C(=O)OC)C2]

To a stirred solution of step B product (1.50 g, 5.88 mmol) and 2-allyl-4,4,5,5-tetramethyl-1,3,2-dioxaborolane (1.48 g, 8.82 mmol) in THF (50 mL) was added Pd(PPh₃)₄ (0.682 g, 0.590 mmol) and cesium carbonate (7.67 g, 23.5 mmol). The reaction was heated at reflux overnight, cooled to room temperature and partitioned between water (80 mL) and ethyl acetate (50 mL). The organic layer was combined with additional extracts (ethyl acetate, 2×50 mL), washed with brine (1×20 mL) and dried over sodium sulfate. The solution was concentrated and the residue was subjected to flash chromatography over silica (5% ethyl acetate in petroleum ether) to afford the title compound as a colorless oil (0.600 g, 47%). MS: 217 m/z [M+H]⁺.

### D. Methyl 5-(3-bromo-2-hydroxypropyl)-2,3-dihydro-1H-indene-2-carboxylate

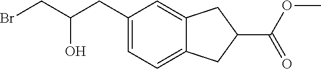 [SMILES: COC(=O)C1Cc2ccc(CC(O)CBr)cc2C1]

To a stirred and cooled (0° C.) solution of step C product (0.650 g, 3.01 mmol) in DMSO (5 mL) was added water (108 μL, 6.02 mmol) and N-bromosuccinimide (0.589 g, 3.31 mmol). The reaction mixture was stirred at 0° C. for another 1.5 hours and then diluted with ethyl acetate (70 mL). The solution was washed with saturated aqueous sodium bicarbonate solution (1×25 mL) and brine (2×20 mL), dried over sodium sulfate and concentrated. The residue was purified by flash chromatography over silica (0-25% ethyl acetate in petroleum ether) to afford the title compound as a colorless oil (0.470 g, 50%). MS: 313, 315 m/z [M+H]⁺.

### E. Methyl 5-(3-bromo-2-oxopropyl)-2,3-dihydro-1H-indene-2-carboxylate

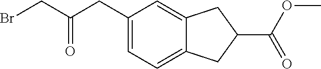 [SMILES: COC(=O)C1Cc2ccc(CC(=O)CBr)cc2C1]

To a stirred solution of step D product (0.470 g, 1.50 mmol) in dichloromethane (15 mL) was added Dess-Martin periodinane (0.955 g, 2.25 mmol). After 6.5 hours at room temperature the reaction was filtered through a pad of Celite, which was subsequently rinsed with additional dichloromethane (2×30 mL). The combined filtrate was washed with saturated aqueous sodium bicarbonate solution (2×20 mL) and brine (2×15 mL), dried over sodium sulfate and concentrated. The residue was purified by flash chromatography over silica (0-15% ethyl acetate in petroleum ether) to afford the title compound as a yellow solid (0.320 g, 68%). MS: 311, 313 m/z [M+H]⁺.

### F. Methyl 5-((2-(2-fluoro-5-((6-fluoro-4-(methylthio)-1H-indol-5-yl)oxy)phenyl)-1H-imidazol-5-yl)methyl)-2,3-dihydro-1H-indene-2-carboxylate

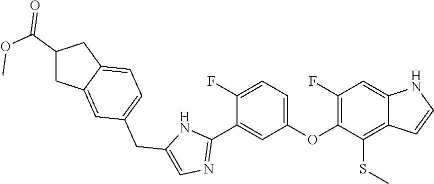 [SMILES: COC(=O)C1Cc2ccc(Cc3cnc(-c4cc(Oc5c(F)cc6[nH]ccc6c5SC)ccc4F)[nH]3)cc2C1]

To a stirred solution of 2-fluoro-5-((6-fluoro-4-(methylthio)-1H-indol-5-yl)oxy)benzimidamide (Intermediate 24-4; 0.302 g, 0.906 mmol) in DMF (5 mL) was added step E product (0.310 g, 0.996 mmol) and potassium carbonate (0.280 g, 2.03 mmol). The reaction mixture was stirred at 75° C. overnight, cooled to room temperature and diluted with ethyl acetate (50 mL), The solution was washed with water (3×20 mL) and brine (1×20 mL), dried over sodium sulfate and concentrated. The residue was purified by flash chromatography over silica (40% petroleum ether in 1:1 dichloromethane/ethyl acetate) to afford the title compound as a white solid (0.292 g, 59%). MS: 546 m/z [M+H]⁺.

### G. Enantiomer 1 and 2 of Methyl 5-((2-(2-fluoro-5-((6-fluoro-4-(methylsulfonyl)-1H-indol-5-yl)oxy)phenyl)-1H-imidazol-5-yl)methyl)-2,3-dihydro-1H-indene-2-carboxylate

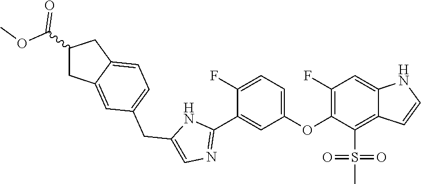 [SMILES: COC(=O)C1Cc2ccc(Cc3cnc(-c4cc(Oc5c(F)cc6[nH]ccc6c5S(C)(=O)=O)ccc4F)[nH]3)cc2C1]

To a stirred solution of step F product (0.200 g, 0.366 mmol) in 1:1 THF/methanol (4 mL) was added a solution of ammonium molybdate tetrahydrate (0.400 g, 0.324 mmol) in 30% aqueous hydrogen peroxide (2 mL). The reaction was stirred at room temperature overnight and then partitioned between ethyl acetate (50 mL) and saturated sodium sulfite solution (50 mL). The organic layer was washed with brine (1×50 mL), dried over sodium sulfate and concentrated. The residue was purified by flash chromatography over silica (0-60% ethyl acetate in petroleum ether) to afford the racemic title compound as a pale white solid (0.110 g, 52%). This material (combined with additional product from a second synthesis; total, 0.160 g) was, in turn, separated into its component enantiomers by chiral SFC (Thar Prep 80 system; 20×250 mm×10 μm CHIRALPAK AD column; column temperature: 35° C.; column pressure: 100 bar; flow rate: 80 mL/min; eluant: 35:65 methanol/CO₂ with 0.2% ammonia/methanol additive).

Enantiomer 1 (first eluting isomer) of the title compound was obtained as a white solid (60.0 mg, 38%, 100% ee).

Enantiomer 2 (second eluting isomer) of the title compound was obtained as a white solid (80.1 mg, 50%, 98.8% ee).

### H. Enantiomer 1 and 2 of 5-((2-(2-Fluoro-5-((6-fluoro-4-(methylsulfonyl)-1H-indol-5-yl)oxy)phenyl)-1H-imidazol-5-yl)methyl)-2,3-dihydro-1H-indene-2-carboxylic acid

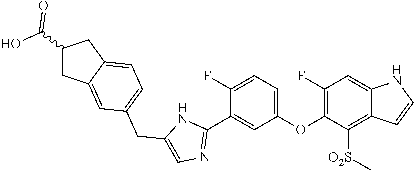 [SMILES: CS(=O)(=O)c1c(Oc2ccc(F)c(-c3ncc(Cc4ccc5c(c4)CC(C(=O)O)C5)[nH]3)c2)c(F)cc2[nH]ccc12]

To a stirred solution of Enantiomer 1 of step G product (60.0 mg, 0.104 mmol) in a mixture of THF (5 mL) and water (1 mL) was added lithium hydroxide (18.0 mg, 0.752 mmol). The reaction was stirred overnight at room temperature and then concentrated to remove the organic solvent. The residue was diluted with water (2 mL) and, with stirring, acidified (˜pH 2) by the dropwise addition of 1.0 N hydrochloric acid. The accumulated precipitate was collected by filtration and purified by preparative HPLC to afford enantiomer 1 of the title compound. Enantiomer 2 of step G (80.1 mg, 0.139 mmol) was hydrolyzed and purified in the same manner to afford enantiomer 2 of the title compound.

Example 83A (enantiomer 1) was obtained as a white solid (37.2 mg, 63%, 97.5% ee). ¹H NMR (400 MHz CD₃OD) δ 7.66 (d, J=10.4 Hz, 1H), 7.54-7.50 (m, 2H), 7.26-7.16 (m, 2H), 7.14-7.07 (m, 2H), 7.05-6.94 (m, 2H), 6.76 (s, 1H), 3.90 (s, 2H), 3.34 (s, 3H), 3.31-3.24 (m, 1H), 3.15 (d, J=7.2 Hz, 4H) ppm. MS: 564 m/z [M+H]⁺.

Example 83B (enantiomer 2) was obtained as a white solid (39.0 mg, 50%, 95.9% ee). ¹H NMR (400 MHz, CD₃OD) δ 7.66 (d, J=10.6 Hz, 1H), 7.54-7.50 (m, 2H), 7.26-7.16 (m, 2H), 7.14-7.07 (m, 2H), 7.05-6.94 (m, 2H), 6.76 (s, 1H), 3.90 (s, 2H), 3.34 (s, 3H), 3.31-3.26 (m, 1H), 3.16 (d, J=7.6 Hz, 4H) ppm. MS: 564 m/z [M+H]⁺.

The following enantiomeric Example compounds were prepared by a synthetic route analogous to that encompassed by steps C-H of Example 83.

| | | | | LCMS | MS |
|---|---|---|---|---|---|
| Structure | Ex. No. | Name | ¹H NMR | Rₜ (min) | (m/z) |
| 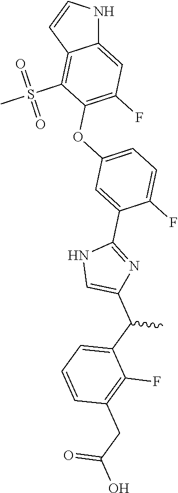 [SMILES: CC(c1c[nH]c(-c2cc(Oc3c(F)cc4[nH]ccc4c3S(C)(=O)=O)ccc2F)n1)c1cccc(CC(=O)O)c1F] | 84 | Enantiomer 1 of 2-(2-fluoro- 3-(1-(2-(2-fluoro-5-((6- fluoro-4-(methylsulfonyl)- 1H-indol-5-yl)oxy)phenyl)- 1H-imidazol-4- yl)ethyl)phenyl)acetic acid | (400 MHz, CD₃OD) δ 7.64 (d, J = 10.5 Hz, 1H), 7.51 (q, J = 2.9 Hz, 2H), 7.16 (td, J = 9.1, 4.3 Hz, 3H), 7.07-6.97 (m, 2H), 6.94 (dt, J = 9.0, 3.5 Hz, 1H), 6.90 (s, 1H), 4.46 (q, J = 7.2 Hz, 1H), 3.57 (s, 2H), 3.32 (s, 3H), 1.58 (d, J = 7.2 Hz, 3H) ppm. | 0.78 | 570 [M + H]⁺ |
| 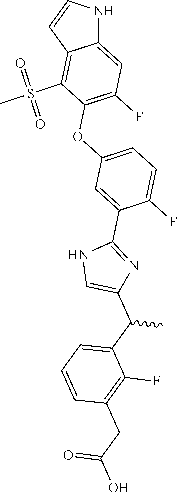 [SMILES: CC(c1c[nH]c(-c2cc(Oc3c(F)cc4[nH]ccc4c3S(C)(=O)=O)ccc2F)n1)c1cccc(CC(=O)O)c1F] | 85 | Enantiomer 2 of 2-(2-fluoro- 3-(1-(2-(2-fluoro-5-((6- fluoro-4-(methylsulfonyl)- 1H-indol-5-yl)oxy)phenyl)- 1H-imidazol-4- yl)ethyl)phenyl)acetic acid | (400 MHz, CD₃OD) δ 7.64 (d, J = 10.5 Hz, 1H), 7.51 (q, J = 2.9 Hz, 2H), 7.16 (td, J = 9.1, 4.3 Hz, 3H), 7.07-6.97 (m, 2H), 6.94 (dt, J = 9.0, 3.5 Hz, 1H), 6.90 (s, 1H), 4.46 (q, J = 7.2 Hz, 1H), 3.57 (s, 2H), 3.32 (s, 3H), 1.58 (d, J = 7.2 Hz, 3H) ppm. | 0.78 | 570 [M + H]⁺ |

### Example 86

### Methyl 3-(3-((2-(3-((6-fluoro-4-(thiazol-2-yl)-1H-indol-5-yl)oxy)phenyl)-1H-imidazol-5-yl)(hydroxy)methyl)phenyl)propanoate

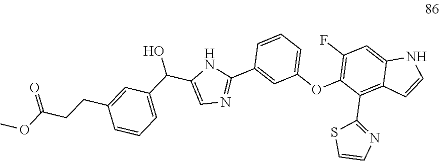 [SMILES: COC(=O)CCc1cccc(C(O)c2cnc(-c3cccc(Oc4c(F)cc5[nH]ccc5c4-c4nccs4)c3)[nH]2)c1]

### A. 3-((6-Fluoro-4-(thiazol-2-yl)-1-tosyl-1H-indol-5-yl)oxy)benzonitrile

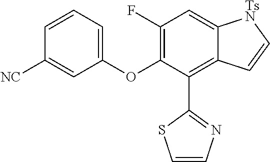 [SMILES: Cc1ccc(S(=O)(=O)n2ccc3c(-c4nccs4)c(Oc4cccc(C#N)c4)c(F)cc32)cc1]

A stirred mixture of 3-((4-bromo-6-fluoro-1-tosyl-1H-indol-5-yl)oxy)benzonitrile (Intermediate 7-2; 2.00 g, 4.12 mmol), 2-(tributylstannyl)thiazole (3.08 g, 8.24 mmol) and Pd(PPh₃)₂Cl₂ (0.579 g, 0.825 mmol) in DMF (20 mL) was heated at 100° C. for three hours. After this time, the reaction was cooled to room temperature and concentrated. The residue was taken up in ethyl acetate (150 mL) and the solution was washed with water (1×150 mL) and brine (1×150 mL) and dried over sodium sulfate. Concentration furnished a residue which was purified by flash chromatography over silica (15% 1:1 ethyl acetate/dichloromethane in petroleum ether). The title compound was obtained as a yellow solid (1.31 g, 65%). MS: 490 m/z [M+H]⁺.

### B. 3-((6-Fluoro-4-(thiazol-2-yl)-1H-indol-5-yl)oxy)benzonitrile

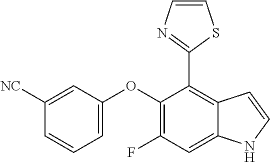 [SMILES: N#Cc1cccc(Oc2c(F)cc3[nH]ccc3c2-c2nccs2)c1]

To a stirred solution of step A product (1.60 g, 3.27 mmol) in THF (20 mL) was added a 1.0 M solution of tetrabutylammonium fluoride in THF (13.1 mL, 13.1 mmol). The reaction was heated at 40° C. for 16 hours, cooled to room temperature and then partitioned between saturated aqueous ammonium chloride solution (40 mL) and ethyl acetate (80 mL). The organic layer was combined with a second extract (ethyl acetate, 1×80 mL), washed with water (1×100 mL) and brine (1×100 mL) and dried over sodium sulfate. The solution was concentrated and the obtained residue was purified by flash chromatography over silica (5% methanol in dichloromethane) to afford the title compound as a yellow solid (0.636 g, 58%). MS: 336 m/z [M+H]⁺.

### C. 3-((6-Fluoro-4-(thiazol-2-yl)-1H-indol-5-yl)oxy)benzimidamide

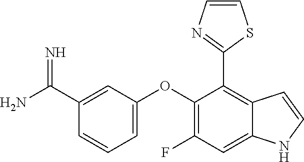 [SMILES: N=C(N)c1cccc(Oc2c(F)cc3[nH]ccc3c2-c2nccs2)c1]

To a stirred and cooled (0° C.) solution of 3-((6-fluoro-4-(thiazol-2-yl)-1H-indol-5-yl)oxy)benzonitrile (0.575 g, 1.72 mmol) in THF (20 mL) was added a 1.0 M solution of lithium bis(trimethylsilyl)amide in THF (6.9 mL, 6.9 mmol). The reaction was heated overnight at 40° C. and then quenched with the addition of saturated aqueous ammonium chloride solution (50 mL). The mixture was extracted with ethyl acetate (4×80 mL) and the combined extracts were washed with brine (1×40 mL), dried over sodium sulfate and concentrated. Crude title compound, which was used without purification, was afforded a yellow solid (0.600 g, 99%). MS: 353 m/z [M+H]⁺.

### D. Example 86, Methyl 3-(3-((2-(3-((6-fluoro-4-(thiazol-2-yl)-1H-indol-5-yl)oxy)phenyl)-1H-imidazol-5-yl)(hydroxy)methyl)phenyl)propanoate

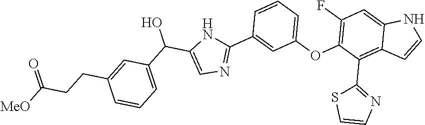 [SMILES: COC(=O)CCc1cccc(C(O)c2cnc(-c3cccc(Oc4c(F)cc5[nH]ccc5c4-c4nccs4)c3)[nH]2)c1]

To a stirred solution of step C (0.105 g, 0.298 mmol) in DMF (14 mL) was added methyl 3-(3-(3-formyloxiran-2-yl)phenyl)propanoate (Intermediate 26-2; 77.0 mg, 0.329 mmol). The reaction was heated overnight at 70° C., cooled to room temperature and diluted with ethyl acetate (80 mL). The solution was washed with water (2×20 mL) and brine (3×20 mL), dried over sodium sulfate and concentrated. The residue was purified by preparative HPLC to afford the title compound as a white solid (20.8 mg, 12%). ¹H NMR (400 MHz, CD₃OD) δ 7.97 (d, J=3.2 Hz, 1H), 7.59-7.58 (m, 1H), 7.53 (d, J=7.6 Hz, 1H), 7.47 (s, 1H), 7.45-7.42 (m, 2H), 7.39 (d, J=3.2 Hz, 1H), 7.37-7.33 (m, 1H), 7.30-7.27 (m, 3H), 7.15 (br s, 1H), 6.91 (dd, J=8.0, 2.0 Hz, 1H), 6.74 (s, 1H), 5.76 (s, 1H), 3.60 (s, 3H), 2.93 (t, J=7.2 Hz, 2H), 2.63 (t, J=7.6 Hz, 2H) ppm. MS: 569 m/z [M+H]⁺.

### Example 87

### 3-(3-((2-(3-((6-Fluoro-4-(thiazol-2-yl)-1H-indol-5-yl)oxy)phenyl)-1H-imidazol-5-yl)(hydroxy)methyl)phenyl)propanoic acid

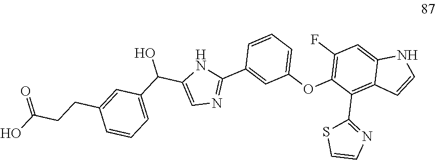 [SMILES: O=C(O)CCc1cccc(C(O)c2cnc(-c3cccc(Oc4c(F)cc5[nH]ccc5c4-c4nccs4)c3)[nH]2)c1]

To a stirred solution of Example 86 compound (10.0 mg, 17.6 μmol) in THF (7 mL) was added a solution of lithium hydroxide (2.0 mg, 48 μmol) in water (0.7 mL). The reaction was heated for two hours at 30° C. and then diluted with water (10 mL) and acidified (˜pH 2) with the dropwise addition of 1.0 N hydrochloric acid. The resulting suspension was extracted with ethyl acetate (3×20 mL) and the combined extracts were concentrated. The residue was purified by preparative HPLC to afford the title compound as a white solid (5.9 mg, 60%). ¹H NMR (400 MHz, CD₃OD) δ 7.98 (d, J=2.8 Hz, 1H), 7.60 (d, J=3.6 Hz, 1H), 7.54 (d, J=7.6 Hz, 1H), 7.48 (s, 1H), 7.45-7.43 (m, 2H), 7.40-7.32 (m, 3H), 7.28-7.27 (m, 2H), 7.18-7.17 (m, 1H), 6.92 (d, J=6.8 Hz, 1H), 6.75 (s, 1H), 5.77 (s, 1H), 2.93 (t, J=7.6 Hz, 2H), 2.59 (t, J=7.6 Hz, 2H) ppm. MS: 555 m/z [M+H]⁺.

### Example 88

### 3-(3-(1-(2-(2-Fluoro-5-((6-fluoro-4-(methylsulfonyl)-1H-indol-5-yl)oxy)phenyl)thiazol-4-yl)-1-hydroxyethyl)phenyl)propanoic acid

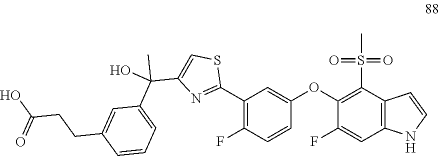 [SMILES: CC(O)(c1cccc(CCC(=O)O)c1)c1csc(-c2cc(Oc3c(F)cc4[nH]ccc4c3S(C)(=O)=O)ccc2F)n1]

### A. (2-(2-Fluoro-5-((6-fluoro-4-(methylthio)-1-tosyl-1H-indol-5-yl)oxy)phenyl)thiazol-4-yl)(3-iodophenyl)methanone

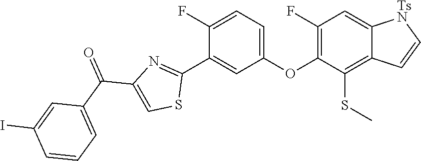 [SMILES: CSc1c(Oc2ccc(F)c(-c3nc(C(=O)c4cccc(I)c4)cs3)c2)c(F)cc2c1ccn2S(=O)(=O)c1ccc(C)cc1]

To a stirred solution of 2-fluoro-5-((6-fluoro-4-(methylthio)-1-tosyl-1H-indol-5-yl)oxy)benzothioamide (Intermediate 33-3; 0.760 g, 1.51 mmol) in ethanol (20 mL) was added 3-bromo-1-(3-iodophenyl)propane-1,2-dione (step A product from the synthesis of Example 37; 0.583 g, 1.65 mmol). The reaction was heated at reflux for two hours and then cooled to room temperature and diluted with water (30 mL). The resulting suspension was extracted with ethyl acetate (3×20 mL) and the combined organic extracts were washed with brine (1×10 mL), dried over sodium sulfate and concentrated. The residue was purified by flash chromatography over silica (0-35% ethyl acetate in petroleum ether) to afford the title compound as a (0.500 g, 44%). MS: 781 m/z [M+Na]⁺.

### B. Ethyl 3-(3-(2-(2-fluoro-5-((6-fluoro-4-(methylthio)-1-tosyl-1H-indol-5-yl)oxy)phenyl)thiazole-4-carbonyl)phenyl)propanoate

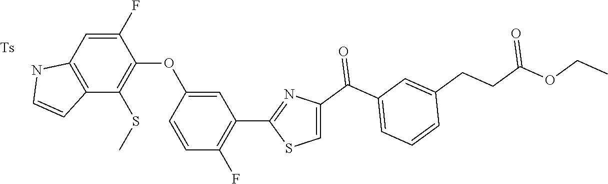 [SMILES: CCOC(=O)CCc1cccc(C(=O)c2csc(-c3cc(Oc4c(F)cc5[nH]ccc5c4SC)ccc3F)n2)c1 ; "Ts"]

To a stirred solution of step A product (0.450 g, 0.593 mmol) in DMF (10 mL) was added 3,3-diethoxyprop-1-ene (0.231 g, 1.78 mmol), tributylamine (282 μL, 1.18 mmol), tetrabutylammonium chloride (0.165 g, 0.594 mmol) and palladium(II) acetate (4.0 mg, 17.8 μmol). The reaction was heated overnight at 90° C., cooled to room temperature and then diluted with water (100 mL). This mixture was extracted with ethyl acetate (3×30 mL) and the combined extracts were washed with brine (1×80 mL), dried over sodium sulfate and concentrated. The residue was purified by flash chromatography over silica (0-50% ethyl acetate in petroleum ether) to the title compound as a yellow solid (0.200 g, 46%). MS: 733 m/z [M+H]⁺.

### C. Ethyl 3-(3-(2-(2-fluoro-5-((6-fluoro-4-(methylsulfonyl)-1-tosyl-1H-indol-5-yl)oxy)phenyl)thiazole-4-carbonyl)phenyl)propanoate

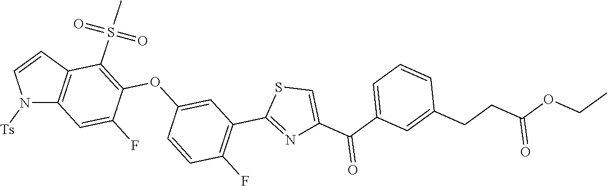 [SMILES: CCOC(=O)CCc1cccc(C(=O)c2csc(-c3cc(Oc4c(F)cc5c(ccn5S(=O)(=O)c5ccc(C)cc5)c4S(C)(=O)=O)ccc3F)n2)c1]

To a stirred solution of step B product (0.134 g, 0.183 mmol) in 1:1 THF/methanol (14 mL) was added a solution of ammonium molybdate tetrahydrate (60 mg) in 30% aqueous hydrogen peroxide (0.3 mL). The reaction was stirred at room temperature for three hours and then diluted with ethyl acetate (70 mL). This solution was washed with water (3×20 mL) and brine (2×10 mL), dried over sodium sulfate and concentrated. The residue was purified by flash chromatography over silica (0-60% ethyl acetate in petroleum ether) to the title compound as a solid (0.143 g, 71%). MS: m/z 765 m/z [M+H]⁺.

### D. 3-(3-(2-(2-Fluoro-5-((6-fluoro-4-(methylsulfonyl)-1-tosyl-1H-indol-5-yl)oxy)phenyl)thiazole-4-carbonyl)phenyl)propanoic acid

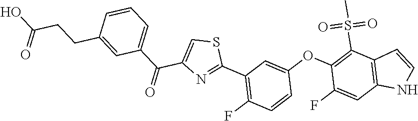 [SMILES: CS(=O)(=O)c1c(Oc2ccc(F)c(-c3nc(C(=O)c4cccc(CCC(=O)O)c4)cs3)c2)c(F)cc2[nH]ccc12]

To a stirred solution of step C product (0.133 g, 0.174 mmol) in 1:1 THF/water (8 mL) was added lithium hydroxide monohydrate (60.0 mg, 1.43 mmol). The reaction was heated at 100° C. for 1.5 hours and then cooled to room temperature and made acidic (˜pH 2) by the dropwise addition of 1N hydrochloric acid. The resulting suspension was diluted with ethyl acetate (50 mL) and the solution was washed with brine (3×10 mL), dried over sodium sulfate and concentrated. The residue was purified by flash chromatography over silica (0-85% ethyl acetate in petroleum ether) to afford the title compound as a solid (89.2 mg, 88%). MS: 583 m/z [M+H]⁺.

### E. Example 88, 3-(3-(1-(2-(2-fluoro-5-((6-fluoro-4-(methylsulfonyl)-1H-indol-5-yl)oxy)phenyl)thiazol-4-yl)-1-hydroxyethyl)phenyl)propanoic acid

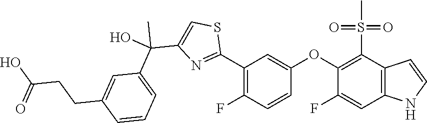 [SMILES: CC(O)(c1cccc(CCC(=O)O)c1)c1csc(-c2cc(Oc3c(F)cc4[nH]ccc4c3S(C)(=O)=O)ccc2F)n1]

To a stirred and cooled (0° C.) solution of step D product (80 mg, 0.14 mmol) in THF (10 mL) with added a 3.0 M solution of methylmagnesium chloride in diethyl ether (0.46 mL, 1.4 mmol). The reaction was warmed to room temperature and stirred for 30 minutes before quenching with the addition of a saturated aqueous ammonium chloride solution (20 mL total, with dropwise addition initially). The mixture was extracted with ethyl acetate (3×20 mL) and the combined extracts were washed with brine (1×50 mL), dried over sodium sulfate and concentrated. The residue was purified by preparative HPLC to afford the title compound as a white solid (14.3 mg, 17%). ¹H NMR (400 MHz, CD₃OD) δ 7.84-7.81 (m, 1H), 7.67 (d, J=10.4 Hz, 1H), 7.56 (d, J=3.2 Hz, 1H), 7.45 (s, 1H), 7.40 (t, J=1.2 Hz, 1H), 7.29 (dt, J=7.6, 1.2 Hz, 1H), 7.25-7.20 (m, 2H), 7.13 (t, J=7.6 Hz, 1H), 7.09-7.07 (m, 1H), 7.01 (dt, J=9.2, 3.6 Hz, 1H), 3.32 (s, 4H), 2.88 (t, J=8.0 Hz, 2H), 2.56 (t, J=8.0 Hz, 2H), 1.92 (s, 3H) ppm. MS: 599 m/z [M+H]⁺.

### Example 89

### 3-(4-((2-(2-Fluoro-5-((6-fluoro-4-methyl-1H-indol-5-yl)oxy)phenyl)-1H-imidazol-4-yl)methyl)thiazol-2-yl)propanoic acid

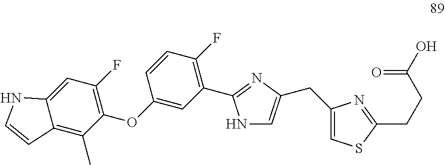 [SMILES: Cc1c(Oc2ccc(F)c(-c3nc(Cc4csc(CCC(=O)O)n4)c[nH]3)c2)c(F)cc2[nH]ccc12]

### A. 3-(4-Bromothiazol-2-yl)propanoic acid

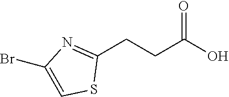 [SMILES: O=C(O)CCc1nc(Br)cs1]

To a stirred and cooled (0° C.) solution of formic acid (0.719 g, 15.6 mmol) was added triethylamine (0.86 mL, 6.2 mmol). The reaction was warmed to room temperature and stirred for 15 min. After this time, the mixture diluted with DMF (10 mL) and treated with 4-bromothiazole-2-carbaldehyde (1.00 g, 5.21 mmol) and 2,2-dimethyl-1,3-dioxane-4,6-dione (0.718 g, 4.98 mmol). The reaction was heated overnight at 100° C. and then diluted water (20 mL) and acidified (pH 1-2) with the dropwise addition of 1.0 N hydrochloric acid. The mixture was extracted with ethyl acetate (3×30 mL) and the combined organic extracts were, in turn, extracted with 1 N aqueous sodium hydroxide (3×10 mL). The combined aqueous layers were acidified (˜pH 2) with concentrated hydrochloric acid and the resulting suspension was extracted with ethyl acetate (3×50 mL). The combined extracts were washed with brine (1×100 mL), dried over sodium sulfate and concentrated to afford the crude title compound, which was used without purification, as a yellow oil (1.10 g, 92%). MS: 236, 238 m/z [M+H]⁺.

### B. Methyl 3-(4-bromothiazol-2-yl)propanoate

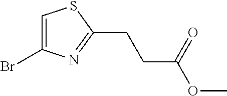

To a stirred solution of step A product (1.10 g, 4.66 mmol) in methanol (15 mL) was slowly added concentrated sulfuric acid (1 mL). The reaction was heated at 80° C. for four hours and then cooled and concentrated. The residue was dissolved in ethyl acetate (100 mL) and this solution was washed with water (3×30 mL) and brine (1×30 mL), dried over sodium sulfate and concentrated. The residue was purified by flash chromatography over silica (10% ethyl acetate in petroleum ether) to afford the title compound as a yellow oil (0.780 g, 60%). MS: 250, 252 m/z [M+H]⁺.

### C. 2-Fluoro-5-((6-fluoro-4-methyl-1-tosyl-1H-indol-5-yl)oxy)-N-(prop-2-yn-1-yl)benzimidamide

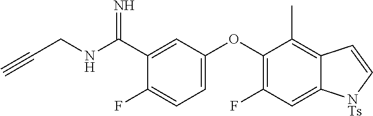 [SMILES: C#CCNC(=N)c1cc(Oc2c(F)cc3c(ccn3S(=O)(=O)c3ccc(C)cc3)c2C)ccc1F]

A 20 mL microwave reaction vessel was loaded with 2-fluoro-5-((6-fluoro-4-methyl-1-tosyl-1H-indol-5-yl)oxy)benzonitrile (Intermediate 7-7; 0.345 g, 0.787 mmol), propargylamine (0.348 g, 6.32 mmol) and THF (6 mL). To this stirred solution was added bis(trimethylaluminum)-1,4-diazabicyclo[2.2.2]octane complex (0.350 mg, 1.38 mmol) and the vessel was sealed and heated in a microwave reactor at 130° C. for 1.5 hours. The reaction was then cooled to room temperature and poured into ice-water (30 mL). The suspension was extracted with ethyl acetate (3×20 mL) and the combined extracts were washed with brine (3×20 mL), dried over sodium sulfate and concentrated. The residue was purified by flash chromatography over silica (0-10% methanol in 98:2 dichloromethane/7N ammonia in methanol solution) to afford the title compound as a yellow solid (0.298 g, 77%). ¹H NMR (400 MHz, DMSO-d₆) δ 7.96 (d, J=8.4 Hz, 2H), 7.88 (d, J=3.8 Hz, 1H), 7.77 (d, J=10.7 Hz, 1H), 7.43 (d, J=8.1 Hz, 2H), 7.17 (t, J=9.6 Hz, 1H), 6.97 (d, J=3.7 Hz, 1H), 6.88-6.86 (m, 2H), 6.84 (br s, 2H) 3.82 (s, 2H), 2.99 (s, 1H), 2.35 (s, 3H), 2.29 (s, 3H) ppm. MS: 494 m/z [M+H]⁺.

### D. Methyl 3-(4-((2-(2-fluoro-5-((6-fluoro-4-methyl-1-tosyl-1H-indol-5-yl)oxy)phenyl)-1H-imidazol-4-yl)methyl)thiazol-2-yl)propanoate

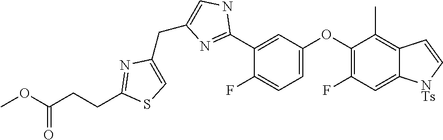 [SMILES: COC(=O)CCc1nc(Cc2c[nH]c(-c3cc(Oc4c(F)cc5c(ccn5S(=O)(=O)c5ccc(C)cc5)c4C)ccc3F)n2)cs1]

To a stirred solution of the step B (0.850 g, 3.64 mmol) and step C products (0.500 g, 1.01 mmol) in DMF (7 mL) was added Pd(PPh₃)₄ (0.116 g, 0.100 mmol), CuI (38.0 mg, 0.200 mmol) and potassium carbonate (0.697 g, 5.04 mmol). The reaction was heated overnight at 80° C. and then cooled to room temperature. After the solids were removed by filtration, the solution was diluted with ethyl acetate (30 mL) and washed with water (2×10 mL) and brine (1×10 mL), dried over sodium sulfate and concentrated. The residue was purified by flash chromatography over silica (33% ethyl acetate in dichloromethane/ethyl acetate) to afford the title compound as a yellow solid (0.150 g, 22%). MS: 663 m/z [M+H]⁺.

### E. 3-(4-((2-(2-Fluoro-5-((6-fluoro-4-methyl-1H-indol-5-yl)oxy)phenyl)-1H-imidazol-4-yl)methyl)thiazol-2-yl)propanoic acid

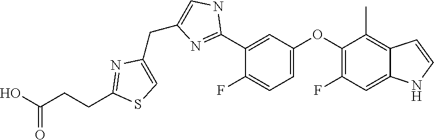 [SMILES: Cc1c(Oc2ccc(F)c(-c3nc(Cc4csc(CCC(=O)O)n4)c[nH]3)c2)c(F)cc2[nH]ccc12]

To a stirred solution of step D product in 1:1 THF/water (4 mL) was added lithium hydroxide (0.200 g, 4.77 mmol). The reaction was heated for 30 minutes at 120° C. in a microwave reactor, cooled to room temperature and diluted with water (10 mL). This stirred solution was acidified (˜pH 2) by the dropwise addition of 1.0 N hydrochloric acid and extracted with ethyl acetate (3×30 mL). The combined extracts were washed with brine (1×50 mL), dried over sodium sulfate and concentrated. The residue was purified by preparative HPLC to afford the title compound as white solid (35.4 mg, 26%). ¹H NMR (400 MHz, CD₃OD) δ 7.42 (s, 1H), 7.28 (s, 1H), 7.18 (t, J=9.2 Hz, 1H), 7.11 (d, J=10.8 Hz, 1H), 7.02 (s, 1H), 6.91-6.87 (m, 2H), 6.53 (s, 1H), 4.07 (s, 2H), 3.28 (t, J=7.2 Hz, 2H), 2.79 (t, J=7.2 Hz, 2H), 2.40 (s, 3H) ppm. MS: 495 m/z [M+H]⁺.

The following Example compounds were prepared by synthetic routes analogous to that described for Example 89.

| | | | | LCMS | MS |
|---|---|---|---|---|---|
| Structure | Ex. No. | Name | ¹H NMR | Rₜ (min) | (m/z) |
| 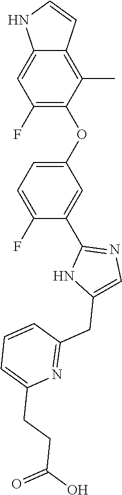 [SMILES: Cc1c(Oc2ccc(F)c(-c3ncc(Cc4cccc(CCC(=O)O)n4)[nH]3)c2)c(F)cc2[nH]ccc12] | 90 | 3-(6-((2-(2-fluoro-5-((6- fluoro-4-methyl-1H-indol-5- yl)oxy)phenyl)-1H-imidazol- 5-yl)methyl)pyridin-2- yl)propanoic acid | (400 MHz, CD₃OD) δ 7.66 (t, J = 7.7 Hz, 1H), 7.42 (dd, J = 6.0, 3.2 Hz, 1H), 7.28 (d, J = 3.2 Hz, 1H), 7.22-7.09 (m, 4H), 6.88 (t, J = 4.4 Hz, 2H), 6.52 (d, J = 3.3 Hz, 1H), 4.12 (s, 2H), 3.08 (t, J = 7.4 Hz, 2H), 2.73 (t, J = 7.5 Hz, 2H), 2.40 (s, 3H) ppm. | 0.80 | 489 [M + H]⁺ |
| 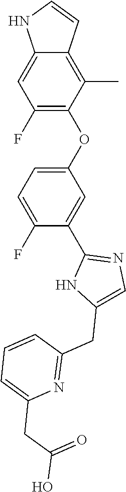 [SMILES: Cc1c(Oc2ccc(F)c(-c3ncc(Cc4cccc(CC(=O)O)n4)[nH]3)c2)c(F)cc2[nH]ccc12] | 91 | 2-(6-((2-(2-fluoro-5-((6- fluoro-4-methyl-1H-indol-5- yl)oxy)phenyl)-1H-imidazol- 5-yl)methyl)pyridin-2- yl)acetic acid | (400 MHz, CD₃OD) δ 7.72-7.69 (m, 1H), 7.43-7.41 (m, 1H), 7.27 (d, J = 3.2 Hz, 2H), 7.21- 7.10 (m, 3H), 6.90-6.85 (m, 2H), 6.52 (d, J = 3.2 Hz, 1H), 4.13 (s, 2H), 3.75 (s, 2H), 2.40 (s, 3H) ppm. | 0.79 | 475 [M + H]⁺ |

### Example 92

### 3-(3-((2-(3-((4-(Dimethylphosphoryl)-6-fluoro-1H-indol-5-yl)oxy)phenyl)thiazol-4-__yl)methy)phenyl)propanoic acid

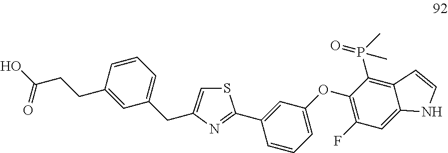 [SMILES: CP(C)(=O)c1c(Oc2cccc(-c3nc(Cc4cccc(CCC(=O)O)c4)cs3)c2)c(F)cc2[nH]ccc12]

### A. Ethyl 3-(3-((2-(3-((4-(dimethylphosphoryl)-6-fluoro-1-tosyl-1H-indol-5-yl)oxy)phenyl)thiazol-4-yl)methyl)phenyl)propanoate

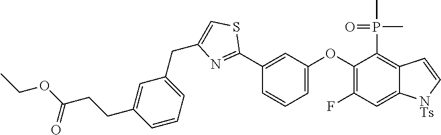 [SMILES: CCOC(=O)CCc1cccc(Cc2csc(-c3cccc(Oc4c(F)cc5c(ccn5S(=O)(=O)c5ccc(C)cc5)c4P(C)(C)=O)c3)n2)c1]

To a stirred solution of 3-((4-(dimethylphosphoryl)-6-fluoro-1-tosyl-1H-indol-5-yl)oxy)benzothioamide (Intermediate 33-2; 0.194 g, 0.376 mmol) in ethanol (5 mL) was added ethyl 3-(3-(3-bromo-2-oxopropyl)phenyl)propanoate (Intermediate 21-7; 0.129 g, 0.412 mmol). The reaction was heated at reflux for three hours and then cooled to room temperature and concentrated. The residue was purified by flash chromatography over silica (20-33% ethyl acetate in petroleum ether) to afford the title compound as a yellow solid (0.171 g, 63%). MS: 731 m/z [M+H]⁺.

### B. Example 92, 3-(3-((2-(3-((4-(Dimethylphosphoryl)-6-fluoro-1-tosyl-1H-indol-5-yl)oxy)phenyl)thiazol-4-yl)methyl)phenyl)propanoic acid

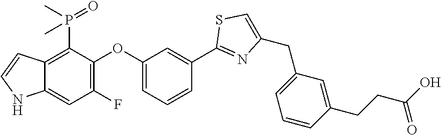 [SMILES: CP(C)(=O)c1c(Oc2cccc(-c3nc(Cc4cccc(CCC(=O)O)c4)cs3)c2)c(F)cc2[nH]ccc12]

A 20 mL microwave reaction vessel was loaded with step A product (0.171 g, 0.234 mmol), potassium carbonate (0.331 g, 2.39 mmol) and methanol (5 mL). The vessel was sealed and heated for 30 minutes at 90° C. in a microwave reactor. The reaction was then cooled to room temperature and concentrated. The residue was purified by preparative HPLC to afford the title compound as a white solid (56 mg, 64%). ¹H NMR (400 MHz, CD₃OD) δ 7.63 (d, J=7.6 Hz, 1H), 7.56 (d, J=10.8 Hz, 1H), 7.51 (s, 1H), 7.48-7.44 (m, 2H), 7.30 (d, J=3.2 Hz, 1H), 7.21-7.17 (m, 2H), 7.11-7.01 (m, 4H), 4.10 (s, 2H), 2.89 (t, J=7.6 Hz, 2H), 2.58 (t, J=7.6 Hz, 2H), 1.87 (s, 3H), 1.83 (s, 3H) ppm. MS: 549 m/z [M+H]⁺.

The following Example compounds were prepared by synthetic routes analogous to that described for Example 92.

| | | | | LCMS | MS |
|---|---|---|---|---|---|
| Structure | Ex. No. | Name | ¹H NMR | Rₜ (min) | (m/z) |
| 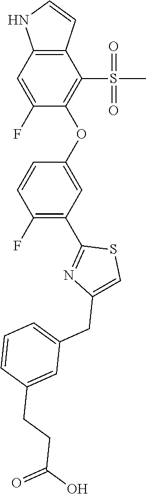 [SMILES: CS(=O)(=O)c1c(Oc2ccc(F)c(-c3nc(Cc4cccc(CCC(=O)O)c4)cs3)c2)c(F)cc2[nH]ccc12] | 93 | 3-(3-((2-(2-fluoro-5-((6- fluoro-4-(methylsulfonyl)- 1H-indol-5- yl)oxy)phenyl)thiazol-4- yl)methyl)phenyl)propanoic acid | (400 MHz, CD₃OD) δ 7.77 (dd, J = 5.8, 3.2 Hz, 1H), 7.68 (d, J = 10.8 Hz, 1H), 7.55 (d, J = 3.2 Hz, 1H), 7.27-7.15 (m, 5H), 7.10-7.02 (m, 3H), 4.11 (s, 2H), 3.34 (s, 3H), 2.88 (t, J = 7.6 Hz, 2H), 2.59 (t, J = 7.6 Hz, 2H) ppm. | — | 569 [M + H]⁺ |
| 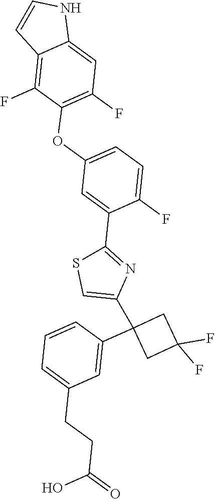 [SMILES: O=C(O)CCc1cccc(C2(c3csc(-c4cc(Oc5c(F)cc6[nH]ccc6c5F)ccc4F)n3)CC(F)(F)C2)c1] | 94 | 3-(3-(1-(2-(5-((4,6-difluoro- 1H-indol-5-yl)oxy)-2- fluorophenyl)thiazol-4-yl)- 3,3- difluorocyclobutyl)phenyl) propanoic acid | (400 MHz, CD₃OD) δ 7.81 (dd, J = 5.7, 3.3 Hz, 1H), 7.34 (d, J = 3.2 Hz, 1H), 7.27-7.16 (m, 5H), 7.12 (d, J = 6.9 Hz, 1H), 7.07- 6.99 (m, 2H), 6.60 (d, J = 3.1 Hz, 1H), 3.47 (dd, J = 25.6, 13.2 Hz, 2H), 3.31-3.20 (m, 2H), 2.91 (t, J = 7.6 Hz, 2H), 2.61 (t, J = 7.6 Hz, 2H) ppm. | — | 585 [M + H]⁺ |
| 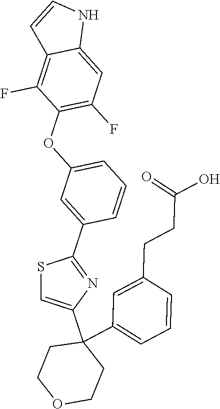 [SMILES: O=C(O)CCc1cccc(C2(c3csc(-c4cccc(Oc5c(F)cc6[nH]ccc6c5F)c4)n3)CCOCC2)c1] | 95 | 3-(3-(4-(2-(3-((4,6-difluoro- 1H-indol-5- yl)oxy)phenyl)thiazol-4- yl)tetrahydro-2H-pyran-4- yl)phenyl)propanoic acid | (400 MHz, CD₃OD) δ 7.59 (d, J = 7.2 Hz, 1H), 7.48 (t, J = 2.0 Hz, 1H), 7.39-7.32 (m, 2H), 7.29 (s, 1H), 7.21-7.15 (m, 4H), 7.06-6.93 (m, 2H), 6.59-6.58 (m, 1H), 3.82-3.65 (m, 4H), 2.88 (t, J = 7.6 Hz, 2H) 2.71-2.67 (m, 2H), 2.57 (t, J = 7.6 Hz, 1H), 2.40-2.33 (m, 2H) ppm. | — | 561 [M + H]⁺ |

### Example 96

### 3-(3-(1-(2-(5-((4-(Dimethylphosphoryl)-6-fluoro-1H-indol-5-yl)oxy)-2-fluorophenyl)-1H-imidazol-5-yl)-1-hydroxyethyl)phenyl)propanoic acid

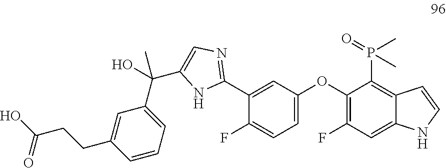 [SMILES: CC(O)(c1cccc(CCC(=O)O)c1)c1cnc(-c2cc(Oc3c(F)cc4[nH]ccc4c3P(C)(C)=O)ccc2F)[nH]1]

### A. Methyl 3-(3-(1-(2-(5-((4-(dimethylphosphoryl)-6-fluoro-1H-indol-5-yl)oxy)-2-fluorophenyl)-1H-imidazol-5-yl)-1-hydroxyethyl)phenyl)propanoate

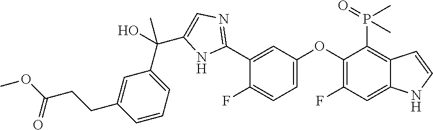 [SMILES: COC(=O)CCc1cccc(C(C)(O)c2cnc(-c3cc(Oc4c(F)cc5[nH]ccc5c4P(C)(C)=O)ccc3F)[nH]2)c1]

A stirred solution of 5-((4-(dimethylphosphoryl)-6-fluoro-1H-indol-5-yl)oxy)-2-fluorobenzimidamide (Intermediate 24-16; 0.178 g, 0.490 mmol) and methyl 3-(3-(3-formyl-2-methyloxiran-2-yl)phenyl)propanoate (Intermediate 26; 0.182 g, 0.733 mmol) in DMF (2 mL) was heated overnight at 80° C. The reaction was then cooled to room temperature, diluted with water (20 mL) and extracted with ethyl acetate (3×25 mL). The combined extracts were washed with water (2×20 mL) and brine (1×20 mL), dried over sodium sulfate and concentrated. The residue was purified by flash chromatography over silica (20-50% ethyl acetate in petroleum ether) to afford the title compound as a brown solid (40 mg, 14%). MS: 594 m/z [M+H]⁺.

### B. Example 96, 3-(3-(1-(2-(5-((4-(Dimethylphosphoryl)-6-fluoro-1H-indol-5-yl)oxy)-2-fluorophenyl)-1H-imidazol-5-yl)-1-hydroxyethyl)phenyl)propanoic acid

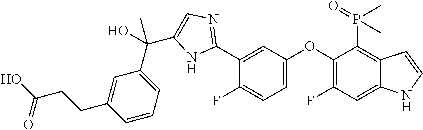 [SMILES: CC(O)(c1cccc(CCC(=O)O)c1)c1cnc(-c2cc(Oc3c(F)cc4[nH]ccc4c3P(C)(C)=O)ccc2F)[nH]1]

To a stirred solution of step A product (20.0 mg, 33.7 μmol) in THF (10 mL) was added a solution of lithium hydroxide (8.0 mg, 0.33 mmol) in water (2 mL). The mixture was stirred at room temperature for six hours and then concentrated. The residue was taken up in water (1 mL) and acidified (˜pH 5) by the dropwise addition 1.0 N hydrochloric acid. The resulting suspension was extracted with ethyl acetate (3×25 mL) and the combined extracts were dried over sodium sulfate and concentrated. The residue was purified by preparative HPLC to the title compound as a white solid (14.9 mg, 77%). ¹H NMR (400 MHz, CD₃OD) δ 7.53 (d, J=11.2 Hz, 1H), 7.51-7.49 (m, 1H), 7.45 (d, J=3.6 Hz, 1H), 7.34 (s, 1H), 7.28-7.18 (m, 4H), 7.13 (d, J=6.8 Hz, 1H), 6.99-6.96 (m, 2H), 2.89 (t, J=8.0 Hz, 2H), 2.44 (t, J=7.6 Hz, 2H), 1.87 (s, 6H), 1.83 (s, 3H) ppm. MS: 580 m/z [M+H]⁺.

### Example 97

### 3-(3-((2-(3-((4-(Dimethylphosphoryl)-6-fluoro-1H-indol-5-yl)oxy)phenyl)thiazol-4-yl)(hydroxy)methyl)phenyl)propanoic acid

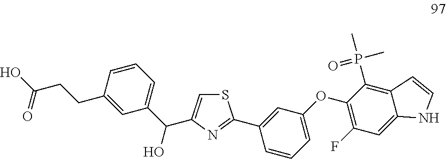 [SMILES: CP(C)(=O)c1c(Oc2cccc(-c3nc(C(O)c4cccc(CCC(=O)O)c4)cs3)c2)c(F)cc2[nH]ccc12]

### A. (2-(3-((4-(Dimethylphosphoryl)-6-fluoro-1-tosyl-1H-indol-5-yl)oxy)phenyl)thiazol-4-yl)(3-iodophenyl)methanone

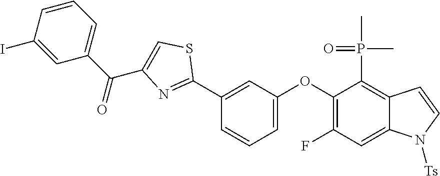 [SMILES: Cc1ccc(S(=O)(=O)n2ccc3c(P(C)(C)=O)c(Oc4cccc(-c5nc(C(=O)c6cccc(I)c6)cs5)c4)c(F)cc32)cc1]

To a stirred solution of 3-((4-(dimethylphosphoryl)-6-fluoro-1-tosyl-1H-indol-5-yl)oxy)benzothioamide (Intermediate 33-2; 1.70 g, 3.29 mmol) in ethanol (40 mL) was added 3-bromo-1-(3-iodophenyl)propane-1,2-dione (step A product from the synthesis of Example 37; 1.16 g, 3.29 mmol). The reaction was heated at reflux for two hours and then cooled and concentrated. The residue was purified by flash chromatography over silica (2-10% methanol in dichloromethane) to afford the title compound as a yellow solid (0.811 g, 32%)). ¹H NMR (400 MHz, CDCl₃) δ 8.74 (t, J=1.6 Hz, 1H), 8.33 (s, 1H), 8.25 (d, J=7.6 Hz, 1H), 8.08 (d, J=10.8 Hz, 1H), 7.93 (d, J=8.0 Hz, 1H), 7.87-7.86 (m, 1H), 7.81 (d, J=8.0 Hz, 2H), 7.72-7.70 (m, 2H), 7.66 (s, 1H), 7.45 (t, J=8.0 Hz, 1H), 7.33-7.30 (m, 2H), 7.22 (t, J=8.0 Hz, 1H), 6.93 (dd, J=8.0, 2.4 Hz, 1H), 2.39 (s, 3H), 1.82 (s, 3H), 1.79 (s, 3H) ppm.

### B. (2-(3-((4-(Dimethylphosphoryl)-6-fluoro-1H-indol-5-yl)oxy)phenyl)thiazol-4-yl)(3-iodophenyl)methanone

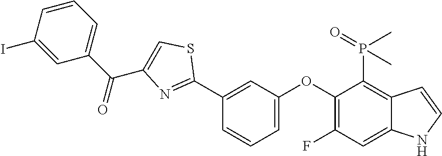 [SMILES: CP(C)(=O)c1c(Oc2cccc(-c3nc(C(=O)c4cccc(I)c4)cs3)c2)c(F)cc2[nH]ccc12]

A 20 mL microwave reaction vessel was loaded with step A product (0.970 g, 1.26 mmol), methanol (10 mL) and potassium carbonate (0.520 g, 3.76 mmol). The vessel was sealed and heated in a microwave reactor for 30 minutes at 80° C. The mixture was then cooled to room temperature and concentrated. The residue was purified by flash chromatography over silica (10-20% ethyl acetate in petroleum ether) to afford the title compound as a yellow solid (0.598 g, 77%). MS: 617 m/z [M+H]⁺.

### C. Methyl (E)-3-(3-(2-(3-((4-(dimethylphosphoryl)-6-fluoro-1H-indol-5-yl)oxy)phenyl)thiazole-4-carbonyl)phenyl)acrylate

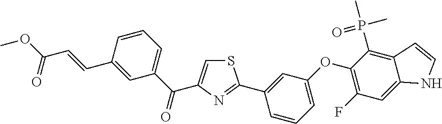 [SMILES: COC(=O)/C=C/c1cccc(C(=O)c2csc(-c3cccc(Oc4c(F)cc5[nH]ccc5c4P(C)(C)=O)c3)n2)c1]

A 20 mL microwave reaction vessel was loaded with step B product (0.598 g, 0.970 mmol), DMF (12 mL), methyl acrylate (272 μL, 3.02 mmol), triphenylphosphine (0.060 g, 0.229 mmol), triethylamine (689 μL, 4.94 mmol) and Pd(OAc)₂ (0.022 g, 98.0 μmol). The vessel was sealed and heated in a microwave reactor for 30 minutes at 110° C. The mixture was then cooled to room temperature and concentrated. The residue was purified by flash chromatography over silica (2-10% methanol in dichloromethane) to afford the title compound as a yellow solid (0.529 g, 95%). MS: 575 m/z [M+H]⁺.

### D. Methyl 3-(3-(2-(3-((4-(dimethylphosphoryl)-6-fluoro-1H-indol-5-yl)oxy)phenyl)thiazole-4-carbonyl)phenyl)propanoate

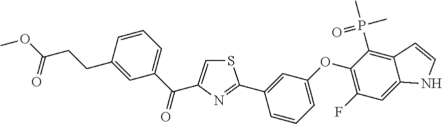 [SMILES: COC(=O)CCc1cccc(C(=O)c2csc(-c3cccc(Oc4c(F)cc5[nH]ccc5c4P(C)(C)=O)c3)n2)c1]

A stirred suspension of step C product (0.529 g, 0.922 mmol) and 10% Pd/C (0.200 g) in methanol (8 mL) was cycled between vacuum and a nitrogen atmosphere three times. The reaction vessel was evacuated a final time and backfilled with hydrogen (via balloon). After two hours at room temperature, the reaction mixture was filtered through a pad of Celite, which was subsequently rinsed with ethyl acetate (˜50 mL). The combined filtrate was concentrated and the residue was purified by flash chromatography over silica (2-10% methanol in dichloromethane) to afford the title compound as a yellow solid (0.207 g, 39%). MS: 577 m/z [M+H]⁺.

### E. Methyl 3-(3-((2-(3-((4-(dimethylphosphoryl)-6-fluoro-1H-indol-5-yl)oxy)phenyl)thiazol-4-yl)(hydroxy)methyl)phenyl)propanoate

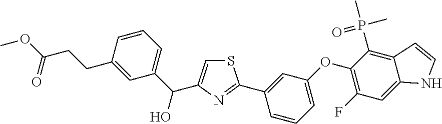 [SMILES: COC(=O)CCc1cccc(C(O)c2csc(-c3cccc(Oc4c(F)cc5[nH]ccc5c4P(C)(C)=O)c3)n2)c1]

To a stirred solution and cooled (0° C.) of step D product (20.0 mg, 34.7 μmol) in methanol (5 mL) was added sodium borohydride (4.0 mg, 106 μmol). The reaction was maintained at 0° C. for 30 minutes and then concentrated. The residue was purified by flash chromatography over silica (2-10% methanol in dichloromethane) to afford the title compound as a white solid (19.9 mg, 99%). MS: 579 m/z [M+H]⁺.

### F. Example 97, 3-(3-((2-(3-((4-(Dimethylphosphoryl)-6-fluoro-1H-indol-5-yl)oxy)phenyl)thiazol-4-yl)(hydroxy)methyl)phenyl)propanoic acid

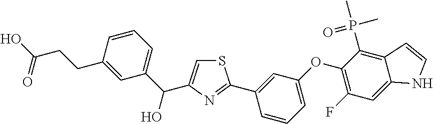 [SMILES: CP(C)(=O)c1c(Oc2cccc(-c3nc(C(O)c4cccc(CCC(=O)O)c4)cs3)c2)c(F)cc2[nH]ccc12]

To a stirred solution of step E product (19.9 mg, 34.4 μmol) in a mixture of THF (3 mL) and water (1 mL) was added lithium hydroxide (5.0 mg, 0.21 mmol). The reaction was stirred at room temperature for two hours and then acidified (˜pH 2) by the dropwise addition of 1.0 N hydrochloric acid. The suspension was concentrated and the residue was purified by flash chromatography over silica (2-10% methanol in dichloromethane) to afford the title compound as a white solid (12.2 mg, 63%). ¹H NMR (400 MHz, CD₃OD) δ 7.63 (d, J=7.6 Hz, 1H), 7.55 (d, J=10.4 Hz, 1H), 7.52 (s, 1H), 7.48-7.42 (m, 2H), 7.34-7.30 (m, 3H), 7.27-7.22 (m, 2H), 7.15 (d, J=7.6 Hz, 1H), 7.02 (d, J=8.4 Hz, 1H), 5.89 (s, 1H), 2.91 (t, J=7.2 Hz, 2H), 2.59 (t, J=7.2 Hz, 2H), 1.85 (s, 3H), 1.82 (s, 3H) ppm. MS: 565 m/z [M+H]⁺.

### Example 98

### 3-(3-(1-(2-(3-((4-(Dimethylphosphoryl)-6-fluoro-1H-indol-5-yl)oxy)phenyl)thiazol-4-yl)-1-hydroxyethyl)phenyl)propanoic acid

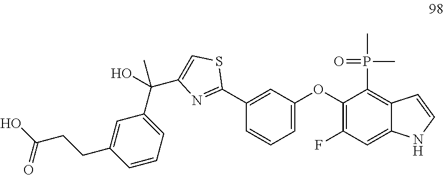 [SMILES: CC(O)(c1cccc(CCC(=O)O)c1)c1csc(-c2cccc(Oc3c(F)cc4[nH]ccc4c3P(C)(C)=O)c2)n1]

### A. Methyl 3-(3-(2-(3-((4-(dimethylphosphoryl)-6-fluoro-1H-indol-5-yl)oxy)phenyl)thiazole-4-carbonyl)phenyl)propanoate

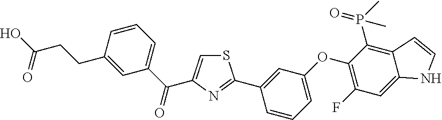 [SMILES: CP(C)(=O)c1c(Oc2cccc(-c3nc(C(=O)c4cccc(CCC(=O)O)c4)cs3)c2)c(F)cc2[nH]ccc12]

To a stirred solution of methyl 3-(3-(2-(3-((4-(dimethylphosphoryl)-6-fluoro-1H-indol-5-yl)oxy)phenyl)thiazole-4-carbonyl)phenyl)propanoate (step D product from the synthesis of Example 97; 0.200 g, 0.347 mmol) in a mixture of THF (3 mL) and water (1 mL) was added lithium hydroxide (50.0 mg, 2.09 mmol). The reaction was stirred at room temperature for two hours and then acidified (˜pH 2) by the dropwise addition of 1.0 N hydrochloric acid. The suspension was concentrated and the residue was purified by flash chromatography over silica (2-10% methanol in dichloromethane) to afford the title compound as a white solid (0.152 g, 78%). MS: 563 m/z [M+H]⁺.

### B. Example 98, 3-(3-(1-(2-(3-((4-(Dimethylphosphoryl)-6-fluoro-1H-indol-5-yl)oxy)phenyl)thiazol-4-yl)-1-hydroxyethyl)phenyl)propanoic acid

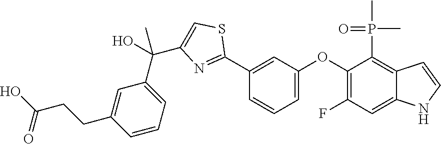 [SMILES: CC(O)(c1cccc(CCC(=O)O)c1)c1csc(-c2cccc(Oc3c(F)cc4[nH]ccc4c3P(C)(C)=O)c2)n1]

To a stirred and cooled (0° C.) solution of step A product (0.130 g, 0.231 mmol) in 1,4-dioxane (2 mL) was added a 3.0 M solution of methylmagnesium bromide in diethyl ether (1.0 mL, 3.0 mmol). The reaction was warmed to room temperature, stirred for an additional one hour and then quenched and acidified (˜pH 2) by the dropwise addition of 1.0 N hydrochloric acid. The mixture was concentrated and the residue was purified by preparative HPLC to afford the title compound as a white solid (0.031 g, 23%). ¹H NMR (400 MHz, CD₃OD) δ 7.64 (d, J=7.6 Hz, 1H), 7.55 (d, J=10.8 Hz, 2H), 7.48 (d, J=3.2 Hz, 1H), 7.45-7.41 (m, 2H), 7.34-7.29 (m, 3H), 7.18 (t, J=8.0 Hz, 1H), 7.09 (d, J=7.6 Hz, 1H), 6.98 (dd, J=2.4, 8.4 Hz, 1H), 2.88 (t, J=7.6 Hz, 2H), 2.56 (t, J=7.6 Hz, 2H), 1.94 (s, 3H), 1.85 (s, 3H), 1.82 (s, 3H) ppm. MS: 579 m/z [M+H]⁺.

### Example 99

### 3-(3-((2-(3-((4-(Hydroxymethyl)-1H-indol-5-yl)oxy)phenyl)thiazol-4-yl)methyl)phenyl)propanoic acid

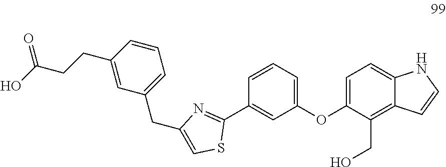 [SMILES: O=C(O)CCc1cccc(Cc2csc(-c3cccc(Oc4ccc5[nH]ccc5c4CO)c3)n2)c1]

### A. 3-((4-(Methoxycarbonyl)-1H-indol-5-yl)oxy)benzimidothioic acid

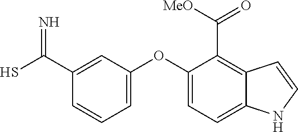 [SMILES: COC(=O)c1c(Oc2cccc(C(=N)S)c2)ccc2[nH]ccc12]

To a stirred slurry of sodium hydrogen sulfide (0.288 g, 5.14 mmol) and magnesium chloride hexahydrate (0.347 g, 1.71 mmol) in DMF (10 mL) was added, in one portion, methyl 5-(3-cyanophenoxy)-1H-indole-4-carboxylate (Intermediate 6-9; 0.500 g, 1.71 mmol). The reaction was stirred at room temperature for 1.5 hours and then poured into water (20 mL). The precipitate which formed was collected by filtration and vacuum oven dried to afford the crude title compound as an amber solid (0.500 g, 90%). This material was used directly in the next reaction without purification.

### B. Methyl 5-(3-(4-(3-iodobenzyl)thiazol-2-yl)phenoxy)-1H-indole-4-carboxylate

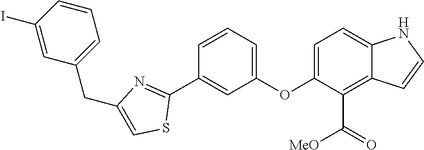 [SMILES: COC(=O)c1c(Oc2cccc(-c3nc(Cc4cccc(I)c4)cs3)c2)ccc2[nH]ccc12]

A stirred mixture of 1-chloro-3-(3-iodophenyl)propan-2-one (0.451 mg, 1.53 mmol) and crude step A product (0.500 g, 1.53 mmol) in ethanol (5 mL) was heated at 70° C. for two hours. The mixture was then concentrated and the residue was purified by flash chromatography over silica (0-100% ethyl acetate in heptane) to afford the crude title compound as a white solid (0.320 g, 37%). MS: 567 m/z [M+H]⁺.

### C. (5-(3-(4-(3-Iodobenzyl)thiazol-2-yl)phenoxy)-1H-indol-4-yl)methanol

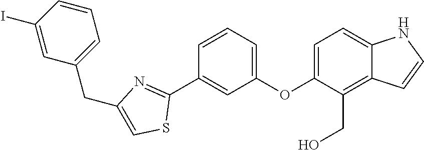 [SMILES: OCc1c(Oc2cccc(-c3nc(Cc4cccc(I)c4)cs3)c2)ccc2[nH]ccc12]

To a stirred and cooled (−78° C.) solution of step B product (0.300 g, 0.530 mmol) in dichloromethane (4 mL) was added a 1.0 M solution of DIBAL-H in hexanes (3.2 mL, 3.2 mmol). The reaction was maintained at −78° C. for one hour and then quenched with the slow addition of a saturated aqueous sodium potassium tartrate solution (15 mL). The mixture was warmed to room temperature and stirred overnight. After this time, the biphasic mixture was extracted with ethyl acetate (3×15 mL). The combined extracts were washed with water (1×30 mL) and brine (1×30 mL), dried over sodium sulfate and concentrated. The residue was purified by flash chromatography over silica (0-100% ethyl acetate in heptane) to afford the title compound as a white solid (0.260 g, 91%). ¹H NMR (400 MHz, CD₃OD) δ 7.65 (d, J=1.9 Hz, 1H), 7.58-7.47 (m, 2H), 7.46-7.29 (m, 4H), 7.26 (d, J=8.9 Hz, 1H), 7.10-6.92 (m, 3H), 6.82 (d, J=8.6 Hz, 1H), 6.71 (d, J=2.2 Hz, 1H), 4.87 (s, 2H), 4.04 (s, 2H) ppm.

### D. Methyl (E)-3-(3-((2-(3-((4-(hydroxymethyl)-1H-indol-5-yl)oxy)phenyl)thiazol-4-yl)methyl)phenyl)acrylate

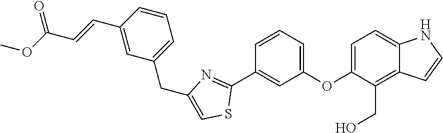 [SMILES: COC(=O)/C=C/c1cccc(Cc2csc(-c3cccc(Oc4ccc5[nH]ccc5c4CO)c3)n2)c1]

A 10 mL microwave reaction vessel was loaded with step C product (0.200 g, 0.371 mmol), DMF (5 mL), triethylamine (517 μl, 3.71 mmol), tri-o-tolylphosphine (22.0 mg, 72.3 μmol), methyl acrylate (43.0 μL, 0.478 mmol) and palladium(II) acetate (4.0 mg, 17.8 μmol). The vessel was sealed and heated for 30 minutes at 100° C. in a microwave reactor. The mixture was then cooled and concentrated. The residue was purified by flash chromatography over silica (0-100% ethyl acetate in heptane) to afford the title compound as a white solid (0.120 g, 65%). ¹H NMR (400 MHz, CDCl₃) δ 8.68-8.62 (m, 1H), 7.67 (d, J=16.0 Hz, 1H), 7.61-7.50 (m, 2H), 7.45-7.36 (m, 2H), 7.36-7.26 (m, 4H), 6.99-6.86 (m, 2H), 6.81-6.73 (m, 2H), 6.43 (d, J=16.0 Hz, 1H), 4.98 (s, 2H), 4.12 (s, 2H), 3.82 (s, 3H), 1.92 (s, 1H) ppm.

### E. Methyl 3-(3-((2-(3-((4-(hydroxymethyl)-1H-indol-5-yl)oxy)phenyl)thiazol-4-yl)methyl)phenyl)propanoate

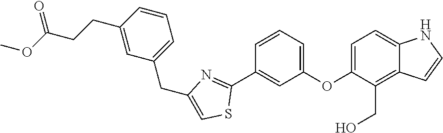 [SMILES: COC(=O)CCc1cccc(Cc2csc(-c3cccc(Oc4ccc5[nH]ccc5c4CO)c3)n2)c1]

A stirred suspension of step C product (0.110 g, 0.220 mmol) and 10% Pd/C (0.047 g) in ethyl acetate (10 mL) was cycled between vacuum and a nitrogen atmosphere three times. The reaction vessel was evacuated a final time and backfilled with hydrogen (via balloon). The reaction was allowed to proceed overnight and then filtered through a pad of Celite, which was subsequently rinsed with ethyl acetate (˜50 mL). The combined filtrate was concentrated and the residue was purified by flash chromatography over silica (0-100% ethyl acetate in petroleum ether) to afford the title compound as a white solid (90 mg, 82%). ¹H NMR (400 MHz, CDCl₃) δ 8.43 (s, 1H), 7.61-7.55 (m, 2H), 7.37-7.18 (m, 3H), 7.14 (d, J=4.2 Hz, 2H), 7.07 (d, J=7.5 Hz, 1H), 6.92 (t, J=8.8 Hz, 2H), 6.79-6.71 (m, 2H), 4.97 (s, 2H), 4.12 (s, 2H), 3.66 (s, 3H), 2.92 (t, J=7.9 Hz, 2H), 2.62 (t, J=7.9 Hz, 2H) ppm. MS: 499 m/z [M+H]⁺.

### F. Example 99, 3-(3-((2-(3-((4-(Hydroxymethyl)-1H-indol-5-yl)oxy)phenyl)thiazol-4-yl)methyl)phenyl)propanoic acid

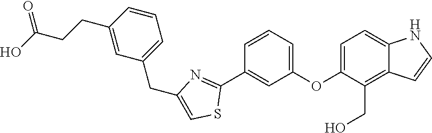 [SMILES: O=C(O)CCc1cccc(Cc2csc(-c3cccc(Oc4ccc5[nH]ccc5c4CO)c3)n2)c1]

To a stirred solution of step E product (80.0 mg, 0.160 mmol) in a mixture of THF (8 mL) and water (3 mL) was added lithium hydroxide (11.5 mg, 0.480 mmol). The reaction was stirred overnight at room temperature and then concentrated. The residue was dissolved in water (2 mL) and this stirred solution was acidified (˜pH 4) with the dropwise addition of 1.0 N hydrochloric acid. The precipitate which formed was collected by filtration and vacuum oven dried to afford the title compound as a white solid (68 mg, 88%). ¹H NMR (400 MHz, CD₃OD) δ 7.52 (ddd, J=7.7, 1.7, 0.9 Hz, 1H), 7.45-7.30 (m, 4H), 7.23-6.96 (m, 7H), 6.82 (d, J=8.7 Hz, 1H), 4.86 (s, 2H), 4.08 (s, 2H), 2.87 (t, J=7.6 Hz, 2H), 2.57 (t, J=7.6 Hz, 2H) ppm (note: peak 4.86 ppm was buried by water peak). MS: 485 m/z [M+H]⁺.

### Example 100

### 3-(3-((2-(5-((4,6-Difluoro-1H-indol-5-yl)oxy)-2-fluorophenyl)-1H-imidazol-5-yl)methyl)-2-fluorophenyl)propanoic acid

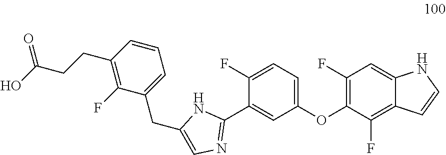 [SMILES: O=C(O)CCc1cccc(Cc2cnc(-c3cc(Oc4c(F)cc5[nH]ccc5c4F)ccc3F)[nH]2)c1F]

### A. 2-(3-(3-Ethoxy-3-oxoprop-1-en-1-yl)-2-fluorophenyl)acetic acid

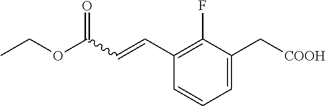 [SMILES: CCOC(=O)C=Cc1cccc(CC(=O)O)c1F]

To a stirred solution of 2-(3-bromo-2-fluorophenyl)acetic acid (4.62 g, 19.8 mmol) in DMF (100 mL) was added ethyl acrylate (4.4 mL. 40 mmol), triethylamine (14.0 mL, 100 mmol), tri(o-tolyl)phosphine (1.41 g, 4.63 mmol) and Pd(OAc)₂ (0.450 g, 2.00 mmol). The reaction was heated overnight at 120° C., cooled to room temperature and diluted with 1.0 N hydrochloric acid. This mixture was extracted with ethyl acetate (3×100 mL). The combined extracts were washed with water (3×200 mL) and brine (1×200 mL), dried over sodium sulfate and concentrated. The residue was purified by flash chromatography over silica (50% ethyl acetate in petroleum ether) to afford the title compound as a white solid (3.50 g, 70%). MS: 253 m/z [M+H]⁺.

### B. 2-(3-(3-Ethoxy-3-oxopropyl)-2-fluorophenyl)acetic acid

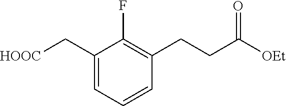 [SMILES: CCOC(=O)CCc1cccc(CC(=O)O)c1F]

A stirred suspension of step A product (3.50 g, 13.9 mmol) and 10% Pd/C (1.00 g) in ethanol (100 mL) was cycled between vacuum and a nitrogen atmosphere three times. The reaction vessel was evacuated a final time and backfilled with hydrogen (via balloon). The reaction was allowed to proceed overnight and then filtered through a pad of Celite, which was subsequently rinsed with ethanol (˜200 mL). The combined filtrate was concentrated and the residue was purified by flash chromatography over silica (50-70% ethyl acetate in hexane) to afford the title compound as a solid (2.20 g, 62%). MS: 255 m/z [M+H]⁺.

### C. Ethyl 3-(3-(3-chloro-2-oxopropyl)-2-fluorophenyl)propanoate

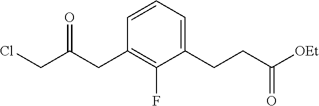 [SMILES: CCOC(=O)CCc1cccc(CC(=O)CCl)c1F]

To a stirred and cooled (0° C.) solution of step B product (1.01 g, 3.97 mmol) in dichloromethane (20 mL) was added oxalyl chloride (0.41 mL, 4.8 mmol) followed by two drops of DMF. The reaction was warmed to room temperature, stirred for two hours and concentrated. The resulting oil was co-evaporated several times with heptane (20 mL) to remove any trace oxalyl chloride. The crude acid chloride was dissolved in 1:1 acetonitrile/THF (20 mL). To this stirred and cooled (0° C.) solution was added, dropwise over five minutes, a 2.0 M solution of trimethylsilyldiazomethane in diethyl ether (8.0 mL, 16 mmol). The reaction was warmed to room temperature, stirred overnight and then concentrated. The crude α-diazoketone was dissolved in dichloromethane (15 mL). To this stirred and cooled (0° C.) solution was added, dropwise over five minutes, a 4.0 M solution of hydrogen chloride in 1,4-dioxane (4.0 mL, 16 mmol; vigorous gas evolution was observed). The reaction was warmed to room temperature, stirred for an additional one hour and then diluted with water (50 mL). This mixture was extracted with ethyl acetate (2×50 mL). The combined extracts were washed with brine (1×50 mL), dried over sodium sulfate and concentrated. The residue was purified by flash chromatography over silica (10-25% ethyl acetate in hexane) to afford the title compound as an oil (0.515 g, 45% over three steps). ¹H NMR (400 MHz, CDCl₃) δ 7.16 (dt, J=13.2, 5.7 Hz, 1H), 7.09-6.99 (m, 2H), 4.18 (s, 2H), 4.12 (q, J=7.2 Hz, 2H), 3.91 (s, 2H), 2.98 (t, J=7.6 Hz, 2H), 2.61 (t, J=7.6 Hz, 2H), 1.23 (t, J=7.2 Hz, 3H) ppm. MS: 287 m/z [M+H]⁺.

### D. 5-(2,6-Difluoro-4-nitrophenoxy)-2-fluorobenzonitrile

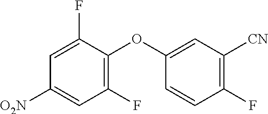 [SMILES: N#Cc1cc(Oc2c(F)cc([N+](=O)[O-])cc2F)ccc1F]

To a stirred solution of 1,2,3-trifluoro-5-nitrobenzene (1.10 g, 6.21 mmol) in DMF (5 mL) was added potassium carbonate (1.71 g, 12.4 mmol) and 2-fluoro-5-hydroxybenzonitrile (0.936 g, 6.83 mmol). The reaction was heated at 100° C. for two hours and then cooled to room temperature and diluted with water (20 mL). The precipitate which formed was collected by suction filtration, washed with water and vacuum oven dried to afford the title compound as a light yellow solid (1.70 g, 94%). ¹H NMR (400 MHz, CDCl₃) δ 8.06-8.01 (m, 2H), 7.30-7.20 (m, 3H) ppm.

### E. 5-(4-Amino-2,6-difluorophenoxy)-2-fluorobenzonitrile

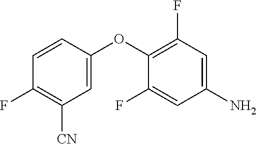 [SMILES: N#Cc1cc(Oc2c(F)cc(N)cc2F)ccc1F]

To a stirred suspension of step D product (1.70 g, 5.78 mmol) in ethanol (30 mL) was added a solution of ammonium chloride (2.45 g, 46.2 mmol) in water (10 mL) followed by iron power (1.48 g, 23.1 mmol). The reaction was heated at reflux for four hours and then cooled to room temperature and filtered to remove the solids. The filtercake was rinsed with additional ethanol (˜30 mL) and the combined filtrate was concentrated. The residue was dissolved in ethyl acetate (100 mL) and this solution was washed with water (3×30 mL) and brine (1×30 mL), dried over sodium sulfate and concentrated. Crude title compound, which was used without purification, was afforded as a yellow solid (1.50 g, 98%). MS: 263 m/z [M−H]⁻.

### F. 5-(4-Amino-2,6-difluoro-3-iodophenoxy)-2-fluorobenzonitrile

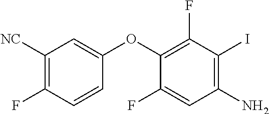 [SMILES: N#Cc1cc(Oc2c(F)cc(N)c(I)c2F)ccc1F]

To a stirred solution of step E product (1.50 g, 5.68 mmol) in acetic acid (12 mL) was added NIS (1.36 g, 6.04 mmol). The reaction was heated at 30° C. for one hour and then concentrated. The residue was purified by flash chromatography over silica (20% ethyl acetate in petroleum ether) to afford the title compound as a yellow solid (1.93 g, 87%). MS: 389 m/z [M−H]⁻.

### G. 5-(4-Amino-2,6-difluoro-3-((trimethylsilyl)ethynyl)phenoxy)-2-fluorobenzonitrile

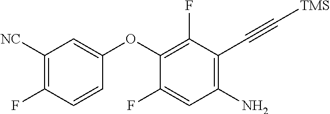 [SMILES: C[Si](C)(C)C#Cc1c(N)cc(F)c(Oc2ccc(F)c(C#N)c2)c1F]

To a stirred solution of step F product (1.00 g, 2.56 mmol) in DMF (15 mL) was added triethylamine (0.54 mL, 3.84 mmol), ethynyltrimethylsilane (0.47 mL, 3.39 mmol), Pd(dppf)Cl₂ (0.182 mg, 0.249 mmol) and CuI (48.0 mg, 0.252 mmol). The reaction was heated at 30° C. for three hours and then partitioned between water (30 mL) and ethyl acetate (30 mL). The organic layer was combined with additional extracts (ethyl acetate, 2×20 mL), washed with brine (1×10 mL) and dried with sodium sulfate. The solution was concentrated and the residue was purified by flash chromatography over silica (20% ethyl acetate in petroleum ether) to afford the title compound as a yellow solid (0.900 g, 98%). MS: 359 m/z [M−H]⁻.

### H. 5-((4,6-Difluoro-1H-indol-5-yl)oxy)-2-fluorobenzonitrile

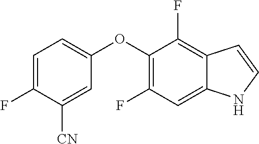 [SMILES: N#Cc1cc(Oc2c(F)cc3[nH]ccc3c2F)ccc1F]

To a stirred solution of step G product (0.900 g, 2.50 mmol) in DMF (5 mL) was added CuI (0.952 g, 5.00 mmol). The reaction was heated overnight at 100° C. After this time, the mixture was cooled to room temperature and filtered free of solids. The filtrate was diluted with ethyl acetate (100 mL) and the solution was washed with brine (1×50 mL), dried over sodium sulfate and concentrated. The residue was purified by flash chromatography over silica (20% ethyl acetate in petroleum ether) to afford the title compound as a yellow solid (0.440 g, 61%). ¹H NMR (400 MHz, DMSO-d₆) δ 11.64 (s, 1H), 7.63-7.61 (m, 1H), 7.51-7.47 (m, 2H), 7.39-7.35 (m, 1H), 7.32 (d, J=10.4 Hz, 1H), 6.58-6.57 (m, 1H) ppm. MS: 287 m/z [M−H]⁻.

### I. 5-((4,6-Difluoro-1H-indol-5-yl)oxy)-2-fluorobenzimidamide

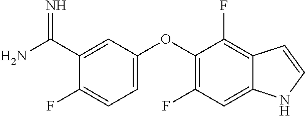 [SMILES: N=C(N)c1cc(Oc2c(F)cc3[nH]ccc3c2F)ccc1F]

To a stirred and cooled (° C.) solution of step H product (0.440 g, 1.53 mmol) in THF (10 mL) was added a 1.0 M solution of lithium bis(trimethylsilyl)amide in THF (6.1 mL, 6.1 mmol). The reaction was allowed to warm to room temperature and stirred overnight. Following this period, the reaction was quenched with addition of saturated aqueous ammonium chloride solution (30 mL) and then extracted with ethyl acetate (2×30 mL). The combined extracts were washed with brine (2×30 mL), dried with sodium sulfate and concentrated. Crude title compound, which was used without purification, was afforded as a light amber solid (0.500 g, 107%). MS: 306 m/z [M+H]⁺.

### J. Ethyl 3-(3-((2-(5-((4,6-difluoro-1H-indol-5-yl)oxy)-2-fluorophenyl)-1H-imidazol-5-yl)methyl)-2-fluorophenyl)propanoate

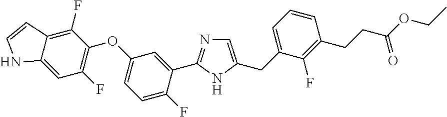 [SMILES: CCOC(=O)CCc1cccc(Cc2cnc(-c3cc(Oc4c(F)cc5[nH]ccc5c4F)ccc3F)[nH]2)c1F]

A stirred solution of step I product (0.100 g, 0.328 mmol), step C product (0.085 g, 0.296 mmol), and sodium bicarbonate (50.0 mg, 0.596 mmol) in DMF (2 mL) was heated overnight at 70° C. The reaction mixture was then cooled to room temperature, diluted with water (50 mL) and extracted with ethyl acetate (3×40 mL). The combined organic extracts were washed with aqueous lithium chloride and aqueous sodium chloride solutions (each, 1×50 mL), dried over sodium sulfate and concentrated. The residue was purified by flash chromatography over silica (50% ethyl acetate in petroleum ether) to afford the title compound as pale yellow oil (0.120 g, 75%). MS: 538 m/z [M+H]⁺.

### K. Example 100, 3-(3-((2-(5-((4,6-Difluoro-1H-indol-5-yl)oxy)-2-fluorophenyl)-1H-imidazol-5-yl)methyl)-2-fluorophenyl)propanoic acid

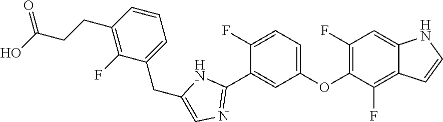 [SMILES: O=C(O)CCc1cccc(Cc2cnc(-c3cc(Oc4c(F)cc5[nH]ccc5c4F)ccc3F)[nH]2)c1F]

To a stirred solution of step J product (2.40 g, 5.20 mmol) in a mixture of THF (39 mL), methanol (13 mL) and water (13 mL) was added lithium hydroxide monohydrate (1.09 g, 26.0 mmol). After two hours at room temperature the mixture was made acidic (˜pH 4) by the addition of 1.0 N hydrochloric acid. The resulting suspension was partitioned between ethyl acetate (100 mL) and brine (1×30 mL). The organic layer was dried over sodium sulfate and concentrated to afford the title compound as white solid (2.40 g, 91%). ¹H NMR (400 MHz, CD₃OD) δ 7.39 (dd, J=6.0, 3.2 Hz, 1H), 7.19 (d, J=3.2 Hz, 1H), 7.12-6.95 (m, 4H), 6.92-6.81 (m, 2H), 6.65 (s, 1H), 6.44 (d, J=2.6 Hz, 1H), 3.86 (s, 2H), 2.84 (t, J=7.6 Hz, 2H), 2.48 (t, J=7.6 Hz, 2H) ppm. MS: 510 m/z [M+H]⁺.

The following Example compounds were prepared by synthetic routes analogous to that described for Example 100.

| | | | | LCMS | LCMS |
|---|---|---|---|---|---|
| Structure | Ex. No. | Name | ¹H NMR | Rₜ (min) | (m/z) |
| 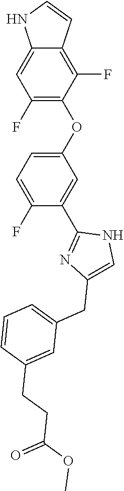 [SMILES: COC(=O)CCc1cccc(Cc2c[nH]c(-c3cc(Oc4c(F)cc5[nH]ccc5c4F)ccc3F)n2)c1] | 101 | methyl 3-(3-((2-(5-((4,6- difluoro-1H-indol-5-yl)oxy)- 2-fluorophenyl)-1H-imidazol- 4- yl)methyl)phenyl)propanoate | (400 MHz, CD₃OD) δ 7.51 (s,1H), 7.30 (d, J = 3.2 Hz, 1H), 7.05-7.22 (m, 6H), 6.98 (dt, J = 3.6, 8.8 Hz, 1H), 6.76 (s, 1H), 6.56 (d, J = 2.8 Hz, 1H), 3.92 (s, 2H), 3.61 (s, 3H), 2.90 (t, J = 7.6 Hz, 2H), 2.61 (t, J = 7.6 Hz, 2H) ppm. | 1.01 | 506 [M + H]⁺ |
| 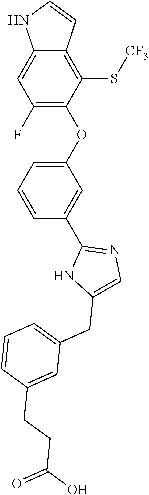 [SMILES: O=C(O)CCc1cccc(Cc2cnc(-c3cccc(Oc4c(F)cc5[nH]ccc5c4SC(F)(F)F)c3)[nH]2)c1] | 102 | 3-(3-((2-(3-((6-fluoro-4- ((trifluoromethyl)thio)-1H- indol-5-yl)oxy)phenyl)-1H- imidazol-5- yl)methyl)phenyl)propanoic acid | (400 MHz, CD₃OD) δ 12.11 (br s, 1H), 11.76 (br s, 1H), 7.76 (d, J = 10.4 Hz, 1H), 7.66-7.64 (m, 1H), 7.45 (t, J = 7.6 Hz, 2H), 7.21 (t, J = 7.6 Hz, 1H), 7.15 (s, 1H), 7.09 (s, 2H), 7.07 (s, 1H), 6.93 (d, J = 8.0 Hz, 1H), 6.66 (s, 1H), 3.92 (s, 2H), 2.79 (t, J = 7.6 Hz, 2H), 2.51 (t, J = 7.6 Hz, 2H) ppm. | 1.00 | 556 [M + H]⁺ |
| 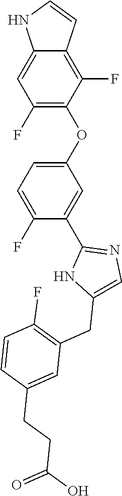 [SMILES: O=C(O)CCc1ccc(F)c(Cc2cnc(-c3cc(Oc4c(F)cc5[nH]ccc5c4F)ccc3F)[nH]2)c1] | 103 | 3-(3-((2-(5-((4,6-difluoro-1H- indol-5-yl)oxy)-2- fluorophenyl)-1H-imidazol-5- yl)methyl)-4- fluorophenyl)propanoic acid | (400 MHz, CD₃OD) δ 7.51 (dd, J = 6.0, 3.2 Hz, 1H), 7.31 (d, J = 3.2 Hz, 1H), 7.21 (dd, J = 10.8, 9.2 Hz, 1H), 7.17-7.11 (m, 3H), 7.01-6.97 (m, 2H), 6.77 (s, 1H), 6.57 (d, J = 3.2 Hz, 1H), 3.96 (s, 2H), 2.86 (t, J = 7.6 Hz, 2H), 2.56 (t, J = 7.6 Hz, 2H) ppm | 0.90 | 510 [M + H]⁺ |
| 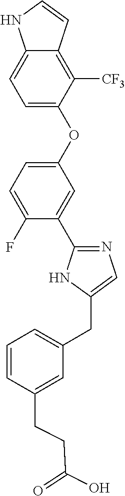 [SMILES: O=C(O)CCc1cccc(Cc2cnc(-c3cc(Oc4ccc5[nH]ccc5c4C(F)(F)F)ccc3F)[nH]2)c1] | 104 | 3-(3-((2-(2-fluoro-5-((4- (trifluoromethyl)-1H-indol-5- yl)oxy)phenyl)-1H-imidazol- 5-yl)methyl)phenyl)propanoic acid | (400 MHz, CD₃OD) δ 7.66 (d, J = 8.8 Hz, 1H), 7.50-7.47 (m, 2H), 7.31-7.03 (m, 7H), 6.92 (d, J = 9.2 Hz, 1H), 6.71-6.68 (m 1H), 4.01 (s, 2H), 2.91 (t, J = 7.6 Hz, 2H), 2.60 (t, J = 7.6 Hz, 2H) ppm. | 0.95 | 524 [M + H]⁺ |
| 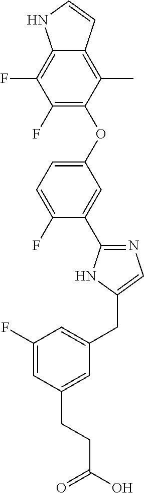 [SMILES: Cc1c(Oc2ccc(F)c(-c3ncc(Cc4cc(F)cc(CCC(=O)O)c4)[nH]3)c2)c(F)c(F)c2[nH]ccc12] | 105 | 3-(3-((2-(5-((6,7-difluoro-4- methyl-1H-indol-5-yl)oxy)-2- fluorophenyl)-1H-imidazol-5- yl)methyl)-5- fluorophenyl)propanoic acid | (400 MHz, CD₃OD) δ 7.42 (dd, J = 3.2, 6.0 Hz, 1H), 7.35 (d, J = 2.8 Hz, 1H), 7.21 (t, J = 6.8 Hz, 1H), 6.92-7.00 (m, 2H), 6.81-6.86 (m, 3H), 6.60 (t, J = 2.0 Hz, 1H), 3.94 (s, 2H), 2.89 (t, J = 7.6 Hz, 2H), 2.59 (t, J = 7.6 Hz, 2H), 2.36 (s, 3H) ppm. | 0.95 | 524 [M + H]⁺ |
| 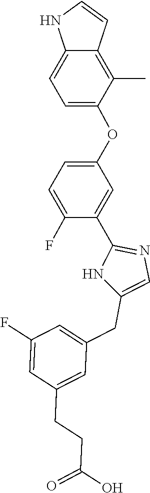 [SMILES: Cc1c(Oc2ccc(F)c(-c3ncc(Cc4cc(F)cc(CCC(=O)O)c4)[nH]3)c2)ccc2[nH]ccc12] | 106 | 3-(3-fluoro-5-((2-(2-fluoro-5- ((4-methyl-1H-indol-5- yl)oxy)phenyl)-1H-imidazol- 5-yl)methyl)phenyl)propanoic acid | (400 MHz, CD₃OD) δ 7.35-7.20 (m, 5H), 7.07 (dt, J = 8.5, 3.8 Hz, 1H), 7.01-6.95 (m, 1H), 6.93-6.77 (m, 3H), 6.52 (ddd, J = 3.1, 2.1, 1.0 Hz, 1H), 4.04 (s, 2H), 2.89 (t, J = 7.5 Hz, 2H), 2.59 (t, J = 7.5 Hz, 2H), 2.34 (d, J = 1.9 Hz, 3H) ppm. | 0.89 | 488 [M + H]⁺ |
| 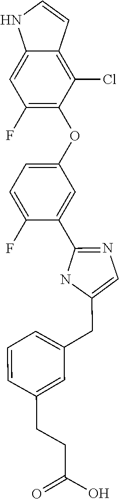 | 107 | 3-(3-((2-(5-((4-chloro-6- fluoro-1H-indol-5-yl)oxy)-2- fluorophenyl)-1H-imidazol-5- yl)methyl)phenyl)propanoic acid | (400 MHz, CD₃OD) δ 7.43 (dd, J = 6.0, 3.2 Hz, 1H), 7.36 (d, J = 3.2 Hz, 1H), 7.27 (d, J = 10.0 Hz, 1H), 7.21-7.13 (m, 3H), 7.07-7.05 (m, 2H), 6.91 (dt, J = 9.2, 3.6 Hz, 1H), 6.74 (s, 1H), 6.55 (d, J = 2.8 Hz, 1H), 3.91 (s, 2H), 2.87 (t, J = 7.6 Hz,2H), 2.55 (t, J = 7.6 Hz, 2H) ppm. | 0.92 | 508 [M + H]⁺ |
| 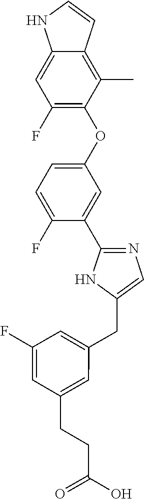 [SMILES: Cc1c(Oc2ccc(F)c(-c3ncc(Cc4cc(F)cc(CCC(=O)O)c4)[nH]3)c2)c(F)cc2[nH]ccc12] | 108 | 3-(3-fluoro-5-((2-(2-fluoro-5- ((6-fluoro-4-methyl-1H-indol- 5-yl)oxy)phenyl)-1H- imidazol-5- yl)methyl)phenyl)propanoic acid | (400 MHz, CD₃OD) δ 7.37 (dd, J = 3.2, 6.0 Hz, 1H), 7.36-7.27 (m, 2H), 7.14-7.11 (m, 2H), 7.05 (dt, J = 3.6, 8.8 Hz,1H), 6.99 (s, 1H), 6.90-6.85 (m, 2H), 6.53 (dd, J = 0.8, 3.6 Hz, 1H), 4.01 (s, 2H), 2.90 (t, J = 7.6 Hz, 2H), 2.60 (t, J = 7.6 Hz, 2H), 2.39 (s, 3H) ppm. | 0.92 | 506 [M + H]⁺ |
| 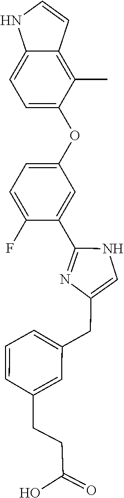 [SMILES: Cc1c(Oc2ccc(F)c(-c3nc(Cc4cccc(CCC(=O)O)c4)c[nH]3)c2)ccc2[nH]ccc12] | 109 | 3-(3-((2-(2-fluoro-5-((4- methyl-1H-indol-5- yl)oxy)phenyl)-1H-imidazol- 4-yl)methyl)phenyl)propanoic acid | (400 MHz, CD₃OD) δ 7.40-7.37 (m, 1H), 7.29-7.24 (m, 2H), 7.23-7.16 (m, 1H), 7.14-7.12 (m, 2H), 7.09-7.07 (m, 2H), 7.86-6.79 (m, 2H), 6.76 (s, 1H), 6.51 (s, 1H), 3.92 (s, 2H), 2.89 (t, J = 7.6 Hz, 2H), 2.57 (t, J = 7.6 Hz, 2H), 2.36 (s, 3H) ppm. | 0.91 | 470 [M + H]⁺ |
| 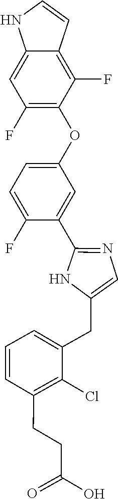 [SMILES: O=C(O)CCc1cccc(Cc2cnc(-c3cc(Oc4c(F)cc5[nH]ccc5c4F)ccc3F)[nH]2)c1Cl] | 110 | 3-(2-chloro-3-((2-(5-((4,6- difluoro-1H-indol-5-yl)oxy)- 2-fluorophenyl)-1H-imidazol- 5-yl)methyl)phenyl)propanoic acid | (400 MHz, CD₃OD) δ 7.53-7.51 (m, 1H), 7.31 (d, J = 3.2 Hz, 1H), 7.24-7.14 (m, 5H), 7.02- 6.98 (m, 1H), 6.75 (s, 1H), 6.56 (d, J = 3.6 Hz, 1H), 4.10 (s, 2H), 3.08 (t, J = 8.0 Hz, 2H), 2.62 (t, J = 8.0 Hz, 2H) ppm. | 0.92 | 526 [M + H]⁺ |
| 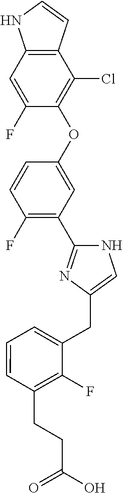 [SMILES: O=C(O)CCc1cccc(Cc2c[nH]c(-c3cc(Oc4c(F)cc5[nH]ccc5c4Cl)ccc3F)n2)c1F] | 111 | 3-(3-((2-(5-((4-chloro-6- fluoro-1H-indol-5-yl)oxy)-2- fluorophenyl)-1H-imidazol-4- yl)methyl)-2- fluorophenyl)propanoic acid | (400 MHz, CD₃OD) δ 7.44 (dd, J = 6.0, 3.2 Hz, 1H), 7.36 (d, J = 3.2 Hz, 1H), 7.27 (d, J = 10.4 Hz, 1H), 7.21-7.12 (m, 2H), 7.08 (t, J = 6.8 Hz, 1H) 7.02- 6.98 (m, 1H), 6.91 (dt, J = 9.2, 3.6 Hz, 1H), 6.76 (s, 1H), 6.55 (d, J = 3.2 Hz, 1H), 3.93 (s, 2H), 2.94 (t, J = 7.6 Hz, 2H), 2.59 (t, J = 7.6 Hz, 2H) ppm. | 0.93 | 526 [M + H]⁺ |
| 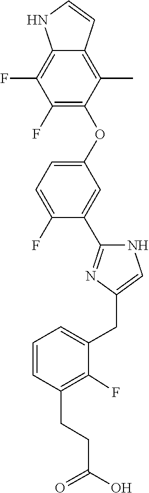 [SMILES: Cc1c(Oc2ccc(F)c(-c3nc(Cc4cccc(CCC(=O)O)c4F)c[nH]3)c2)c(F)c(F)c2[nH]ccc12] | 112 | 3-(3-((2-(5-((6,7-difluoro-4- methyl-1H-indol-5-yl)oxy)-2- fluorophenyl)-1H-imidazol-4- yl)methyl)-2- fluorophenyl)propanoic acid | (400 MHz, CD₃OD) δ 7.42-7.40 (m, 1H), 7.36 (d, J = 2.8 Hz, 1H), 7.25 (t, J = 10.0 Hz, 1H), 7.18 (t, J = 7.2 Hz, 1H), 7.11 (t, J = 6.4 Hz, 1H), 7.05-6.97 (m, 2H), 6.89 (s, 1H), 6.59 (t, J = 3.2 Hz, 1H), 4.01 (s, 2H), 2.96 (t, J = 7.6 Hz, 2H), 2.61 (t, J = 7.6 Hz, 2H), 2.36 (s, 3H) ppm. | 0.93 | 524 [M + H]⁺ |
| 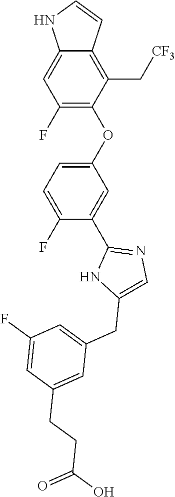 [SMILES: O=C(O)CCc1cc(F)cc(Cc2cnc(-c3cc(Oc4c(F)cc5[nH]ccc5c4CC(F)(F)F)ccc3F)[nH]2)c1] | 113 | 3-(3-fluoro-5-((2-(2-fluoro-5- ((6-fluoro-4-(2,2,2- trifluoroethyl)-1H-indol-5- yl)oxy)phenyl)-1H-imidazol- 5-yl)methyl)phenyl)propanoic acid | (400 MHz,CD₃OD) δ7.44 (dd, J = 6.0, 3.2 Hz, 1H), 7.38 (d, J = 3.2 Hz, 1H), 7.33 (d, J = 10.8 Hz, 1H), 7.27 (dd, J = 10.4, 9.6 Hz, 1H), 7.04-6.99 (m, 3H), 6.86 (t, J = 9.6 Hz, 2H), 6.60 (d, J = 3.2 Hz, 1H), 4.00 (s, 2H), 3.77 (q, J = 10.8 Hz, 2H), 2.91 (t, J = 7.6 Hz, 2H), 2.60 (t, J = 7.6 Hz, 2H) ppm. | 0.98 | 574 [M + H]⁺ |
| 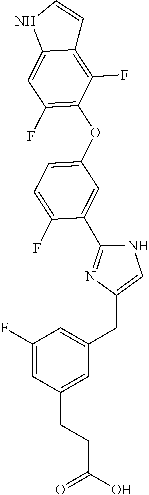 [SMILES: O=C(O)CCc1cc(F)cc(Cc2c[nH]c(-c3cc(Oc4c(F)cc5[nH]ccc5c4F)ccc3F)n2)c1] | 114 | 3-(3-((2-(5-((4,6-difluoro-1H- indol-5-yl)oxy)-2- fluorophenyl)-1H-imidazol-4- yl)methyl)-5- fluorophenyl)propanoic acid | (400 MHz, CD₃OD) δ 7.52 (m, 1H), 7.31 (d, J = 3.2 Hz, 1H), 7.23-7.13 (m, 2H), 7.01-6.97 (m, 2H), 6.84-6.80 (m, 3H), 6.56 (d, J = 2.8 Hz, 1H), 3.93 (s, 2H), 2.91 (t, J = 7.6 Hz, 2H), 2.64 (t, J = 7.6 Hz, 1H) ppm. | 0.92 | 510 [M + H]⁺ |
| 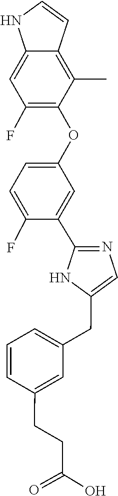 [SMILES: Cc1c(Oc2ccc(F)c(-c3ncc(Cc4cccc(CCC(=O)O)c4)[nH]3)c2)c(F)cc2[nH]ccc12] | 115 | 3-(3-((2-(2-fluoro-5-((6- fluoro-4-methyl-1H-indol-5- yl)oxy)phenyl)-1H-imidazol- 5-yl)methyl)phenyl)propanoic acid | (400 MHz, CD₃OD) δ 7.40 (dd, J = 6.0, 3.2 Hz, 1H), 7.27 (d, J = 3.2 Hz, 1H), 7.23-7.07 (m, 6H), 6.88 (dt, J = 8.8, 3.6 Hz, 1H), 6.76 (s, 1H), 6.52 (d, J = 3.2 Hz, 1H), 3.92 (s, 2H), 2.89 (t, J = 7.6 Hz, 2H), 2.57 (t, J = 7.6 Hz, 2H), 2.39 (s, 3H) ppm | 0.91 | 488 [M + H]⁺ |
| 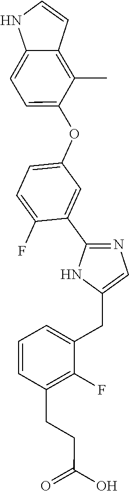 [SMILES: Cc1c(Oc2ccc(F)c(-c3ncc(Cc4cccc(CCC(=O)O)c4F)[nH]3)c2)ccc2[nH]ccc12] | 116 | 3-(2-fluoro-3-((2-(2-fluoro-5- ((4-methyl-1H-indol-5- yl)oxy)phenyl)-1H-imidazol- 5-yl)methyl)phenyl)propanoic acid | (400 MHz, CD₃OD) δ 7.37 (dd, J = 6.0, 2.8 Hz, 1H), 7.26 (d, J = 3.2 Hz, 1H), 7.24 (d, J = 8.4 Hz, 1H), 7.24-7.18 (m, 2H), 7.07 (t, J = 7.2 Hz, 1H), 6.99 (t, J = 7.6 Hz, 1H), 6.87 (dt, J = 9.2, 3.6 Hz, 1H), 6.81 (s, 1H), 6.78 (d, J = 8.4 Hz, 1H), 6.49 (d, J = 3.2 Hz, 1H), 3.96 (s, 2H), 2.95 (t, J = 7.6 Hz, 2H), 2.57 (t, J = 7.6 Hz, 2H), 2.33 (s, 3H) ppm. | 0.88 | 488 [M + H]⁺ |
| 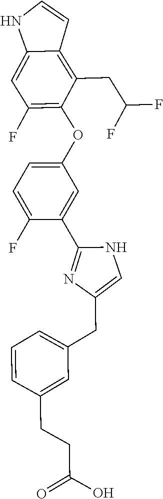 [SMILES: O=C(O)CCc1cccc(Cc2c[nH]c(-c3cc(Oc4c(F)cc5[nH]ccc5c4CC(F)F)ccc3F)n2)c1] | 117 | 3-(3-((2-(5-((4-(2,2- difluoroethyl)-6-fluoro-1H- indol-5-yl)oxy)-2- fluorophenyl)-1H-imidazol-4- yl)methyl)phenyl)propanoic acid | (400 MHz, CD₃OD) δ 7.45 (dd, J = 6.0, 3.2 Hz, 1H), 7.34 (d, J = 3.2 Hz, 1H), 7.27-7.17 (m, 3H), 7.14 (s, 1H), 7.09 (d, J = 7.2 Hz, 2H), 6.92 (dt, J = 9.2, 3.6 Hz, 1H), 6.78 (s, 1H), 6.59 (d, J = 2.8 Hz, 1H), 6.11 (tt, J = 57.2, 4.8 Hz, 1H), 3.93 (s, 2H), 3.42 (td, J = 16.8, 4.8 Hz, 2H), 2.89 (t, J = 7.6 Hz, 2H), 2.58 (t, J = 7.6 Hz, 2H) ppm. | 0.91 | 538 [M + H]⁺ |
| 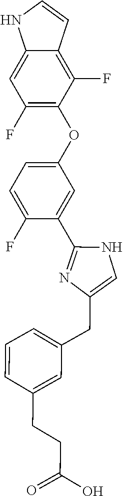 [SMILES: O=C(O)CCc1cccc(Cc2c[nH]c(-c3cc(Oc4c(F)cc5[nH]ccc5c4F)ccc3F)n2)c1] | 118 | 3-(3-((2-(5-((4,6-difluoro-1H- indol-5-yl)oxy)-2- fluorophenyl)-1H-imidazol-4- yl)methyl)phenyl)propanoic acid | (400 MHz, CD₃OD) δ 7.49 (dd, J = 6.0, 2.8 Hz, 1H), 7.31 (d, J = 3.2 Hz, 1H), 7.23-7.14 (m, 4H), 7.08 (t, J = 7.2 Hz, 2H), 6.98 (dt, J = 9.2, 3.2 Hz, 1H), 6.76 (s, 1H), 6.56 (d, J = 3.2 Hz, 1H), 3.92 (s, 2H), 2.88 (t, J = 7.6 Hz, 2H), 2.54 (t, J = 7.6 Hz, 2H) ppm. | 0.91 | 492 [M + H]⁺ |
| 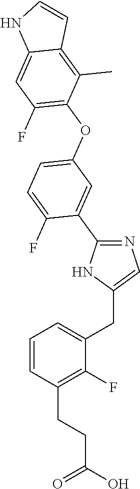 [SMILES: Cc1c(Oc2ccc(F)c(-c3ncc(Cc4cccc(CCC(=O)O)c4F)[nH]3)c2)c(F)cc2[nH]ccc12] | 119 | 3-(2-fluoro-3-((2-(2-fluoro-5- ((6-fluoro-4-methyl-1H-indol- 5-yl)oxy)phenyl)-1H- imidazol-5- yl)methyl)phenyl)propanoic acid | (400 MHz, CD₃OD) δ 7.42 (dd, J = 6.1, 3.1 Hz, 1H), 7.28 (d, J = 3.1 Hz, 1H), 7.22-7.00 (m, 5H), 6.90 (dd, J = 8.1, 4.3 Hz, 1H), 6.80 (s, 1H), 6.53 (d, J = 3.1 Hz, 1H), 3.99 (s, 2H), 2.96 (t, J = 7.6 Hz, 2H), 2.61 (t, J = 7.7 Hz, 2H), 2.40 (s, 3H) ppm. | 0.90 | 506 [M + H]⁺ |
| 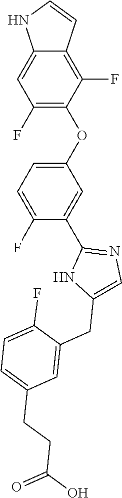 [SMILES: O=C(O)CCc1ccc(F)c(Cc2cnc(-c3cc(Oc4c(F)cc5[nH]ccc5c4F)ccc3F)[nH]2)c1] | 120 | 3-(3-((2-(5-((4,6-difluoro-1H- indol-5-yl)oxy)-2- fluorophenyl)-1H-imidazol-5- yl)methyl)-4- fluorophenyl)propanoic acid | (400 MHz, CD₃OD) δ 7.51 (dd, J = 6.0, 2.8 Hz, 1H), 7.31 (d, J = 3.2 Hz, 1H), 7.21 (dd, J = 10.8, 9.2 Hz, 1H), 7.17-7.10 (m, 3H), 7.01-6.97 (m, 2H), 6.77 (s, 1H), 6.57 (d, J = 3.2 Hz, 1H), 3.96 (s, 2H), 2.86 (t, J = 7.6 Hz, 2H), 2.56 (t, J = 7.6 Hz, 2H) ppm. | 0.89 | 510 [M + H]⁺ |
| 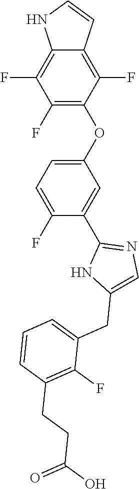 [SMILES: O=C(O)CCc1cccc(Cc2cnc(-c3cc(Oc4c(F)c(F)c5[nH]ccc5c4F)ccc3F)[nH]2)c1F] | 121 | 3-(2-fluoro-3-((2-(2-fluoro-5- ((4,6,7-trifluoro-1H-indol-5- yl)oxy)phenyl)-1H-imidazol- 5-yl)methyl)phenyl)propanoic acid | (400 MHz, CD₃OD) δ 7.53 (dd, J = 5.9, 3.2 Hz, 1H), 7.39 (d, J = 3.0 Hz, 1H), 7.28-7.20 (m, 1H), 7.16 (t, J = 7.0 Hz, 1H), 7.12-6.96 (m, 3H), 6.78 (s, 1H), 6.64 (t, J = 2.9 Hz, 1H), 3.98 (s, 2H), 2.96 (t, J = 7.7 Hz, 2H), 2.61 (t, J = 7.6 Hz, 2H) ppm. | 0.92 | 528 [M + H]⁺ |
| 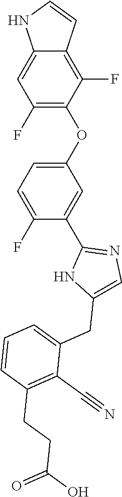 [SMILES: N#Cc1c(CCC(=O)O)cccc1Cc1cnc(-c2cc(Oc3c(F)cc4[nH]ccc4c3F)ccc2F)[nH]1] | 122 | 3-(2-cyano-3-((2-(5-((4,6- difluoro-1H-indol-5-yl)oxy)- 2-fluorophenyl)-1H-imidazol- 5-yl)methyl)phenyl)propanoic acid | (400 MHz, CD₃OD) 8 7.57 (m, 1H), 7.49-7.47 (m, 1H), 7.43- 7.31 (m, 4H), 7.22-7.16 (m, 3H), 6.56 (d, J = 2.8 Hz, 1H), 4.31 (s, 2H), 3.17 (t, J = 7.6 Hz, 2H), 2.72 (t, J = 7.2 Hz, 2H) ppm. | 0.89 | 517 [M + H]⁺ |
| 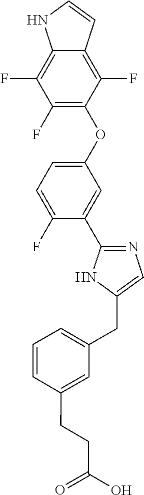 [SMILES: O=C(O)CCc1cccc(Cc2cnc(-c3cc(Oc4c(F)c(F)c5[nH]ccc5c4F)ccc3F)[nH]2)c1] | 123 | 3-(3-((2-(2-fluoro-5-((4,6,7- trifluoro-1H-indol-5- yl)oxy)phenyl)-1H-imidazol- 5-yl)methyl)phenyl)propanoic acid | (400 MHz, CD₃OD) δ 7.52 (dd, J = 6.0, 3.2 Hz, 1H), 7.39 (d, J = 3.2 Hz, 1H), 7.17-7.28 (m, 2H), 7.14 (s, 1H), 7.00-7.11 (m, 3H), 6.76 (s, 1H), 6.64 (t, J = 3.2 Hz, 1H), 3.93 (s, 2H), 2.89 (t, J = 7.6 Hz, 2H), 2.57 (t, J = 7.6 Hz, 2H) ppm. | 0.87 | 510 [M + H]⁺ |
| 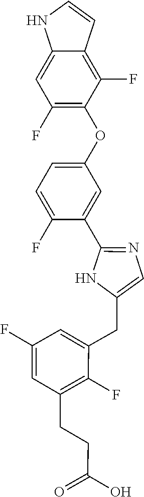 [SMILES: O=C(O)CCc1cc(F)cc(Cc2cnc(-c3cc(Oc4c(F)cc5[nH]ccc5c4F)ccc3F)[nH]2)c1F] | 124 | 3-(3-((2-(5-((4,6-difluoro-1H- indol-5-yl)oxy)-2- fluorophenyl)-1H-imidazol-5- yl)methyl)-2,5- difluorophenyl)propanoic acid | (400 MHz, CD₃OD) δ 7.51 (d, J = 6.0 Hz, 1H), 7.31 (d, J = 3.2 Hz, 1H), 7.24-7.12 (m, 2H), 7.01-6.90 (m, 2H), 6.87-6.75 (m, 2H), 6.56 (d, J = 3.2 Hz, 1H), 3.95 (s, 2H), 2.94 (t, J = 7.6 Hz, 2H), 2.52 (t, J = 7.6 Hz, 2H) ppm. | | 528 [M + H]⁺ |
| 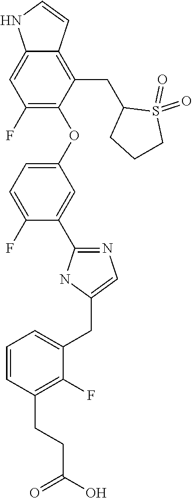 | 125 | 3-(3-((2-(5-((4-((1,1- dioxidotetrahydrothiophen-2- yl)methyl)-6-fluoro-1H-indol- 5-yl)oxy)-2-fluorophenyl)- 1H-imidazol-5-yl)methyl)-2- fluorophenyl)propanoic acid | (400 MHz, CD₃OD) δ 7.46 (dd, J = 6.0, 2.8 Hz, 1H), 7.35 (d, J = 2.8 Hz, 1H), 7.26-7.13 (m, 3H), 7.11-7.07 (m, 1H), 7.01 (t, J = 7.6 Hz, 1H), 6.94 (dd, J = 8.8, 3.6 Hz, 1H), 6.77 (s, 1H), 6.61 (d, J = 2.8 Hz, 1H), 3.98 (s, 2H), 3.41-3.38 (m, 1H), 3.16-3.05 (m, 4H), 2.96 (t, J = 7.2 Hz, 2H), 2.60 (t, J = 7.2 Hz, 2H), 2.20-1.86 (m, 4H) ppm | | 624 [M + H]⁺ |
| 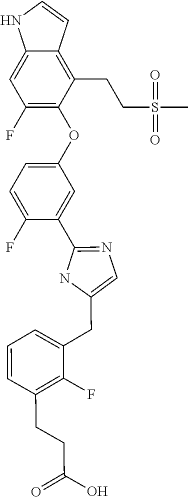 | 126 | 3-(2-fluoro-3-((2-(2-fluoro-5- ((6-fluoro-4-(2- (methylsulfonyl)ethyl)-1H- indol-5-yl)oxy)phenyl)-1H- imidazol-5- yl)methyl)phenyl)propanoic acid | (400 MHz, CD₃OD) δ 7.44 (dd, J = 6.0, 3.2 Hz, 1H), 7.36 (d, J = 3.2 Hz, 1H), 7.24 (d, J = 4.0 Hz, 1H), 7.21 (d, J = 2.4 Hz, 1H), 7.19-7.14 (m, 1H), 7.08 (t, J = 6.8 Hz, 1H), ), 7.03 (d, J = 7.6 Hz, 1H), 6.99-6.96 (m, 1H), 6.77 (s, 1H), 6.63 (d, J = 2.8 Hz, 1H), 3.97 (s, 2H), 3.38-3.30 (m,4H), 2.96 (t, J = 7.6 Hz, 2H), 2.89 (s, 3H), 2.57 (t, J = 7.6 Hz, 2H) ppm. | 0.78 | 598 [M + H]⁺ |

### Example 127

### 3-(3-((2-(5-((4,6-Difluoro-1H-indol-5-yl)oxy)-2-fluorophenyl)-1H-imidazol-4-yl)methyl)phenyl)propanamide

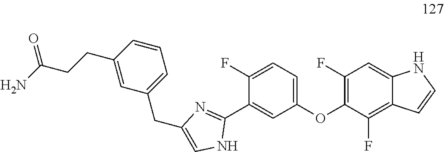 [SMILES: NC(=O)CCc1cccc(Cc2c[nH]c(-c3cc(Oc4c(F)cc5[nH]ccc5c4F)ccc3F)n2)c1]

To a stirred solution of Example 118 compound (50.0 mg, 0.102 mmol), ammonium chloride (16.0 mg, 0.299 mmol) and HATU (62.0 mg, 0.163 mmol) in DMF (8 mL) was added, dropwise, diisopropylethylamine (0.89 mL, 0.51 mmol). After three hours at room temperature, the reaction was partitioned between water (30 mL) and ethyl acetate (30 mL), The organic layer was combined with additional extracts (ethyl acetate, 2×30 mL), washed with brine (3×20 mL) and dried over sodium sulfate. The solution was concentrated and the residue was purified by preparative HPLC to afford the title compound as a white solid (8.7 mg, 17%). ¹H NMR (400 MHz, CD₃OD) δ 7.51 (s, 1H), 7.31 (d, J=3.2 Hz, 1H), 7.22-7.14 (m, 4H), 7.08 (d, J=7.2 Hz, 2H), 6.97 (dt, J=8.8, 3.6 Hz, 1H), 6.76 (s, 1H), 6.56 (d, J=2.8 Hz, 1H), 3.92 (s, 2H), 2.88 (t, J=7.6 Hz, 2H), 2.48 (t, J=7.6 Hz, 2H) ppm. MS: 491 m/z [M+H]⁺.

### Example 128

### Enantiomer 1 of 3-(2-fluoro-3-(1-(2-(2-fluoro-5-((6-fluoro-4-(2,2,2-trifluoroethyl)-1H-indol-5-yl)oxy)phenyl)-1H-imidazol-5-yl)ethyl)phenyl)propanoic acid (128A)

### And

### Enantiomer 2 of 3-(2-fluoro-3-(1-(2-(2-fluoro-5-((6-fluoro-4-(2,2,2-trifluoroethyl)-1H-indol-5-yl)oxy)phenyl)-1H-imidazol-5-yl)ethyl)phenyl)propanoic acid (128B)

### Absolute Configurations Unknown

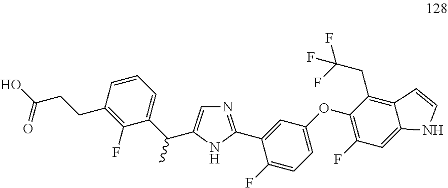 [SMILES: CC(c1cnc(-c2cc(Oc3c(F)cc4[nH]ccc4c3CC(F)(F)F)ccc2F)[nH]1)c1cccc(CCC(=O)O)c1F]

### A. Ethyl 3-(2-fluoro-3-(1-(2-(2-fluoro-5-((6-fluoro-4-(2,2,2-trifluoroethyl)-1H-indol-5-yl)oxy)phenyl)-1H-imidazol-4-yl)ethyl)phenyl)propanoate

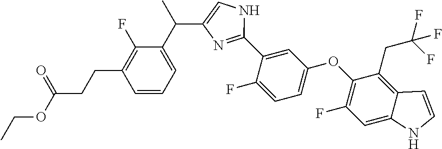 [SMILES: CCOC(=O)CCc1cccc(C(C)c2c[nH]c(-c3cc(Oc4c(F)cc5[nH]ccc5c4CC(F)(F)F)ccc3F)n2)c1F]

Exchanging 5-((4,6-difluoro-1H-indol-5-yl)oxy)-2-fluorobenzimidamide (Intermediate 24-9) for 2-fluoro-5-[[6-fluoro-4-(2,2,2-trifluoroethyl)-1H-indol-5-yl]oxy]benzamidine (Intermediate 24-18; 0.400 g, 1.08 mmol) and ethyl 3-(3-(3-chloro-2-oxopropyl)-2-fluorophenyl)propanoate for ethyl 3-[3-(3-chloro-1-methyl-2-oxo-propyl)-2-fluoro-phenyl]propanoate (Intermediate 21-9; 0.260 g, 0.864 mmol), a procedure analogous to that described in step J of the Example 100 synthesis (imidazole formation from an amidine and an α-chloroketone) was used to prepare the title compound as a yellow solid (0.360 g, 68%). MS: 616 m/z [M+H]⁺.

### B. Enantiomers 1 and 2 of ethyl 3-(2-fluoro-3-(1-(2-(2-fluoro-5-((6-fluoro-4-(2,2,2-trifluoroethyl)-1H-indol-5-yl)oxy)phenyl)-1H-imidazol-4-yl)ethyl)phenyl)propanoate

Racemic step A product (0.360 g, 0.584 mmol) was separated into its component enantiomers by chiral SFC (Thar Prep 80 system; 20×250 mm×10 μm (R,R)Whelk-O1 column; flow rate: 80 mL/min; column temperature: 35° C.; column pressure: 100 bar; eluant: 50:50 CO₂/ethanol with 0.2% methylethylamine modifier).

Enantiomer 1 (first eluting isomer) of the title compound was obtained a white solid (100 mg, 28%). MS: 616 m/z [M+H]⁺.

Enantiomer 2 (second eluting isomer) of the title compound was obtained a white solid (100 mg, 28%). MS: 616 m/z [M+H]⁺.

### C. Enantiomer 1 and 2 of 3-(2-fluoro-3-(1-(2-(2-fluoro-5-((6-fluoro-4-(2,2,2-trifluoroethyl)-1H-indol-5-yl)oxy)phenyl)-1H-imidazol-5-yl)ethyl)phenyl)propanoic acid

Exchanging ethyl 3-(3-((2-(5-((4,6-difluoro-1H-indol-5-yl)oxy)-2-fluorophenyl)-1H-imidazol-5-yl)methyl)-2-fluorophenyl)propanoate for the separate step B products, Enantiomer 1 (0.100 g, 0.162 mmol) and Enantiomer 2 (0.100 g, 0.162 mmol), the reaction conditions described in step K of the Example 100 synthesis were used to prepare the title compounds (Example 128A and Example 128B, respectively).

Example 128A (enantiomer 1) was obtained as a white solid (95 mg, 99%). MS: 588 m/z [M+H]⁺. ¹H NMR (400 MHz, CD₃OD) δ 7.43 (dd, J=5.6, 2.8 Hz, 1H), 7.39 (d, J=3.2 Hz, 1H), 7.35 (d, J=3.6 Hz, 1H), 7.32 (dd, J=10.8, 7.6 Hz, 2H), 7.24-7.20 (m, 1H), 7.11-7.04 (m, 3H), 6.60 (d, J=3.2 Hz, 1H), 4.56 (q, J=6.8 Hz, 1H), 3.76 (q, J=10.8 Hz, 2H), 2.97 (d, J=7.6 Hz, 2H), 2.61 (d, J=7.6 Hz, 2H), 1.66 (d, J=7.2 Hz, 3H) ppm.

Example 128B (enantiomer 2) was obtained as a white solid (95 mg, 99%). MS 588 m/z [M+H]⁺. ¹H NMR (400 MHz, CD₃OD) δ 7.43 (dd, J=6.0, 3.2 Hz, 1H), 7.38 (d, J=3.2 Hz, 1 H), 7.31 (t, J=10.0 Hz, 2H), 7.28-7.25 (m, 1H), 7.21-7.18 (m, 1H), 7.08-7.01 (m, 3H), 6.60 (d, J=3.2 Hz, 1H), 4.56 (q, J=7.2 Hz, 1H), 3.76 (q, J=10.8 Hz, 2H), 2.97 (d, J=7.6 Hz, 2H), 2.61 (d, J=7.6 Hz, 2H), 1.66 (d, J=7.2 Hz, 3H) ppm.

### Example 131A and 131B

### Enantiomers 1 and 2 of 2-(3-(1-(2-(5-((4,6-difluoro-1H-indol-5-yl)oxy)-2-fluorophenyl)-1H-imidazol-5-yl)ethyl)-2-fluorophenyl)acetic acid

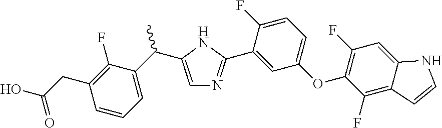 [SMILES: CC(c1cnc(-c2cc(Oc3c(F)cc4[nH]ccc4c3F)ccc2F)[nH]1)c1cccc(CC(=O)O)c1F]

### A. Ethyl 2-(3-(1-(2-(5-((4,6-difluoro-1H-indol-5-yl)oxy)-2-fluorophenyl)-1H-imidazol-5-yl)ethyl)-2-fluorophenyl)acetate

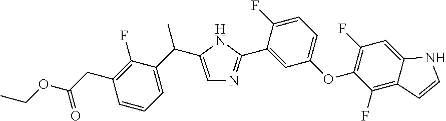 [SMILES: CCOC(=O)Cc1cccc(C(C)c2cnc(-c3cc(Oc4c(F)cc5[nH]ccc5c4F)ccc3F)[nH]2)c1F]

To a stirred solution of ethyl 2-(3-(4-bromo-3-oxobutan-2-yl)-2-fluorophenyl)acetate (Intermediate 44, 18.2 g, 54.8 mmol) in N,N-dimethylformamide (120 mL) was added sodium bicarbonate (9.20 μg, 109.6 mmol) and 5-((4,6-difluoro-1H-indol-5-yl)oxy)-2-fluorobenzimidamide (Intermediate 24-9, 16.72 g, 54.8 mmol). The mixture was stirred overnight at 75° C., cooled to room temperature, and quenched with water (400 ml). The solution was extracted with ethyl acetate (3×400 mL). The combined organic layers were washed with brine (2×200 mL), dried over sodium sulfate, and concentrated. The residue was purified by silica gel chromatography eluted with petroleum ether/ethyl acetate (1/1) to afford the title compound (14.32 g, 48.5%) as a light-yellow oil. Mass: 538 m/z [M+H]⁺; Retention time: 1.81 minutes; purity: 97% (254 nm) (LCMS Method: Mobile Phase: A: water (0.1% trifluoroacetic acid) B: acetonitrile (0.1% trifluoroacetic acid); Gradient: 10% B increase to 90% B within 1.3 minutes, 90% B for 1.5 minutes, back to 5% B within 0.01 minutes. Flow Rate: 2 mL/minute; Column: Sunfire C18, 4.6×50 mm, 3.5 μm; Column Temperature: 50° C.).

### B. Enantiomers 1 and 2 of ethyl 2-(3-(1-(2-(5-((4,6-difluoro-1H-indol-5-yl)oxy)-2-fluorophenyl)-1H-imidazol-5-yl)ethyl)-2-fluorophenyl)acetate

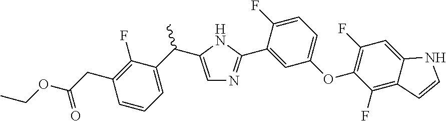 [SMILES: CCOC(=O)Cc1cccc(C(C)c2cnc(-c3cc(Oc4c(F)cc5[nH]ccc5c4F)ccc3F)[nH]2)c1F]

Ethyl 2-(3-(1-(2-(5-((4,6-difluoro-1H-indol-5-yl)oxy)-2-fluorophenyl)-1H-imidazol-5-yl)ethyl)-2-fluorophenyl)acetate (Step A product, 14.32 g, 26.6 mmol) was subject to chiral SFC separation under the following conditions: Instrument: SFC-80 (Thar, Waters); Column: SSWHELK 20×250 mm, 10 μm (Daicel); Column temperature: 35° C.; Mobile phase: carbon dioxide/ethanol=45/55; Flow rate: 80 g/minute; Back pressure: 100 bar; Detection wavelength: 214 nm; Cycle time: 5 minutes; Sample solution: 14.32 g dissolved in 450 mL of methanol; Injection volume: 2 mL. The first eluent is Enantiomer 1 (5.7 g, 40% yield, 100% ee). The second eluent is Enantiomer 2 (6 g, 41.9% yield, 100% ee).

### C. Example 131A: Enantiomers 1 of 2-(3-(1-(2-(5-((4,6-difluoro-1H-indol-5-yl)oxy)-2-fluorophenyl)-1H-imidazol-5-yl)ethyl)-2-fluorophenyl)acetic acid

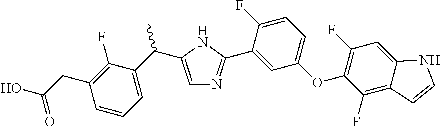 [SMILES: CC(c1cnc(-c2cc(Oc3c(F)cc4[nH]ccc4c3F)ccc2F)[nH]1)c1cccc(CC(=O)O)c1F]

To a stirred solution of Enantiomer 1 of ethyl 2-(3-(1-(2-(5-((4,6-difluoro-1H-indol-5-yl)oxy)-2-fluorophenyl)-1H-imidazol-5-yl)ethyl)-2-fluorophenyl)acetate (5.7 g, 10.6 mmol) in tetrahydrofuran/water/methanol (30 mL/10 mL/10 mL) was added lithium hydroxide monohydrate (1.34 g, 32 mmol). The reaction was stirred at room temperature for 2 hours, then acidified to pH˜4 with 1M hydrochloric acid. The solution was extracted with ethyl acetate (3×60 mL). The combined organic extracts were washed with brine, dried over sodium sulfate, filtered, and concentrated to give the pure title product (4.99 g, 92%) as a white solid. MS (ESI): 510 m/z [M+H]⁺; Retention time: 1.65 minutes; Purity: >99% (LC-MS Method: Mobile Phase: A: water (0.1% formic acid) B: Acetonitrile (0.1% formic acid); Gradient: 10% B increase to 90% B within 1.3 minutes, 90% B for 1.5 minutes, back to 5% B within 0.01 minutes; Flow Rate: 2 mL/minute; Column: Sunfire C18, 4.6×50 mm, 3.5 μm). Chiral purity: >99% (>99% ee); Retention time: 2.4 minutes (Chiral SFC conditions: Mobile phase: carbon dioxide/methanol (0.2% 7M ammonia in methanol)=70:30; Column: (R,R)-Welk-O1, 4.6×100 mm, 5 μm; Column temperature: 40° C.; back pressure: 120 bar; Injection volume: 5 μL). ¹H NMR (400 MHz, CD₃OD) δ 7.46 (dd, J=5.6, 3.2 Hz, 1H), 7.31 (d, J=3.2 Hz, 1H), 7.27-7.02 (m, 7H), 6.54 (d, J=3.2 Hz, 1H), 4.52 (q, J=7.2 Hz, 1H), 3.68 (s, 2H), 1.62 (d, J=7.2 Hz, 3H) ppm.

### D. Example 131B: Enantiomers 2 of 2-(3-(1-(2-(5-((4,6-difluoro-1H-indol-5-yl)oxy)-2-fluorophenyl)-1H-imidazol-5-yl)ethyl)-2-fluorophenyl)acetic acid

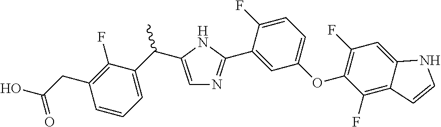 [SMILES: CC(c1cnc(-c2cc(Oc3c(F)cc4[nH]ccc4c3F)ccc2F)[nH]1)c1cccc(CC(=O)O)c1F]

Utilizing identical procedure as described in Step C, Enantiomer 2 (5.7 g, 10.6 mmol) of ethyl 2-(3-(1-(2-(5-((4,6-difluoro-1H-indol-5-yl)oxy)-2-fluorophenyl)-1H-imidazol-5-yl)ethyl)-2-fluorophenyl)acetate was hydrolyzed to afford the title compound (5.36 g, 99%) as a white solid. MS (ESI): 510 m/z [M+H]⁺; Purity: 95% (214 nm) (Same LC-MS method as Step C). Chiral purity: 98.4% (97% ee), Retention time: 3.56 minutes (same SFC conditions as Step C). ¹H NMR (400 MHz, CD₃OD) δ 7.46 (dd, J=5.6, 3.2 Hz, 1H), 7.31 (d, J=3.2 Hz, 1H), 7.27-7.02 (m, 7H), 6.54 (d, J=3.2 Hz, 1H), 4.42 (q, J=7.2 Hz, 1H), 3.68 (s, 2H), 1.62 (d, J=7.2 Hz, 3H) ppm.

The following Examples, which are also single enantiomers of unknown absolute configuration, were prepared by a three-step process analogous to that used to generate the Example 128A/128B enantiomer pair: racemic ester synthesis, chromatographic resolution, and parallel hydrolysis the enantiomers. Modifications to the chromatographic conditions used for ester resolution are specified in the annotations.

| | | | | LCMS | MS |
|---|---|---|---|---|---|
| Structure | Ex. No. | Name | ¹H NMR | Rₜ (min) | (m/z) |
| 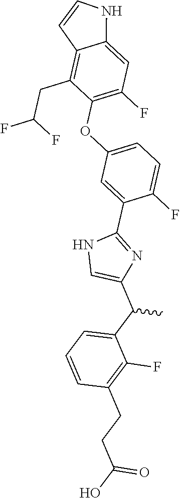 [SMILES: CC(c1c[nH]c(-c2cc(Oc3c(F)cc4[nH]ccc4c3CC(F)F)ccc2F)n1)c1cccc(CCC(=O)O)c1F] | 129A^{a} | Enantiomer 1 of 3-(3-(1-(2- (5-((4-(2,2-difluoroethyl)-6- fluoro-1H-indol-5-yl)oxy)-2- fluorophenyl)-1H-imidazol-4- yl)ethyl)-2- fluorophenyl)propanoic acid | (400 MHz, CD₃OD) δ 7.48 (s, 1H), 7.41 (dd, J = 5.6, 2.8 Hz, 1H), 7.39-7.35 (m, 2H), 7.28 (d, J = 11.2 Hz, 1H), 7.25-7.24 (m, 1H), 7.16-7.07 (m, 3H), 6.61 (d, J = 2.8 Hz, 1H), 6.13 (tt, J = 56.8, 4.8 Hz, 1H), 4.61 (q, J = 6.4 Hz, 1H), 3.43 (td, J = 16.8, 5.2 Hz, 2H), 2.98 (t, J = 7.6 Hz, 2H), 2.63 (t, J = 7.6 Hz, 2H), 1.70 (d, J = 6.8 Hz, 3H) ppm. | 0.94 | 570 [M + H]⁺ |
| 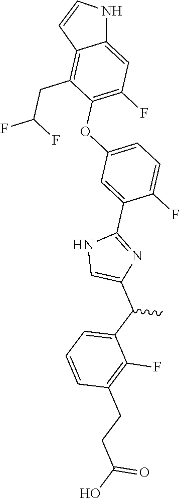 [SMILES: CC(c1c[nH]c(-c2cc(Oc3c(F)cc4[nH]ccc4c3CC(F)F)ccc2F)n1)c1cccc(CCC(=O)O)c1F] | 129B^{a} | Enantiomer 2 of 3-(3-(1-(2- (5-((4-(2,2-difluoroethyl)-6- fluoro-1H-indol-5-yl)oxy)-2- fluorophenyl)-1H-imidazol-4- yl)ethyl)-2- fluorophenyl)propanoic acid | | 0.94 | 570 [M + H]⁺ |
| 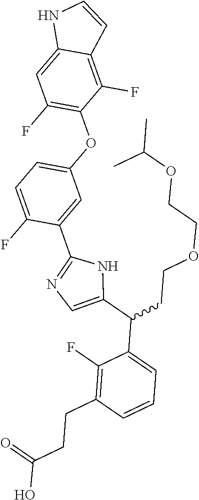 [SMILES: CC(C)OCCOCCC(c1cnc(-c2cc(Oc3c(F)cc4[nH]ccc4c3F)ccc2F)[nH]1)c1cccc(CCC(=O)O)c1F] | 130A^{b} | Enantiomer 1 of 3-(3-(1-(2- (5-((4,6-difluoro-1H-indol-5- yl)oxy)-2-fluorophenyl)-1H- imidazol-5-yl)-3-(2- isopropoxyethoxy)propyl)-2- fluorophenyl)propanoic acid | (400 MHz, CD₃OD) δ 7.50-7.48 (m, 1H), 7.31 (d, J = 3.2 Hz, 1H), 7.22-7.13 (m, 4H), 7.03 (t, J = 7.6 Hz, 1H), 6.98-6.96 (m, 2H), 6.56 (d, J = 3.2 Hz, 1H), 4.55 (t, J = 8.0 Hz, 1H), 3.64- 3.58 (m, 1H), 3.55-3.45 (m, 6H), 2.96 (t, J = 7.6 Hz, 2H), 2.60 (t, J = 7.6 Hz, 2H), 2.43- 2.35 (m, 1H), 2.24-2.17 (m, 1H), 1.13 (d, J = 6.4 Hz, 6H) ppm. | | 640 [M + H]⁺ |
| 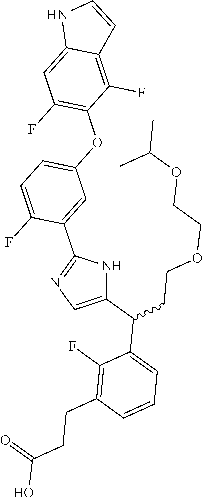 [SMILES: CC(C)OCCOCCC(c1cnc(-c2cc(Oc3c(F)cc4[nH]ccc4c3F)ccc2F)[nH]1)c1cccc(CCC(=O)O)c1F] | 130B^{b} | Enantiomer 2 of 3-(3-(1-(2- (5-((4,6-difluoro-1H-indol-5- yl)oxy)-2-fluorophenyl)-1H- imidazol-5-yl)-3-(2- isopropoxyethoxy)propyl)-2- fluorophenyl)propanoic acid | (400 MHz, CD₃OD) δ 7.50-7.48 (m, 1H), 7.31 (d, J = 2.8 Hz, 1H), 7.22-7.13 (m, 4H), 7.03 (t, J = 7.6 Hz, 1H), 7.01-6.96 (m, 2H), 6.56 (d, J = 2.8 Hz, 1H), 4.55 (t, J = 7.2 Hz, 1H), 3.64- 3.58 (m, 1H), 3.55-3.45 (m, 6H), 2.96 (t, J = 7.6 Hz, 2H), 2.60 (t, J = 7.6 Hz, 2H), 2.41- 2.36 (m, 1H), 2.23-2.19 (m, 1H), 1.13 (d, J = 6.4 Hz, 6H) ppm. | | 640 [M + H]⁺ |
| 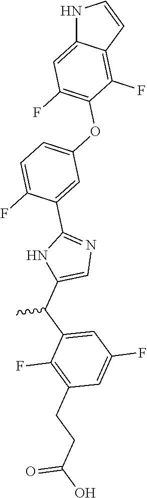 [SMILES: CC(c1cnc(-c2cc(Oc3c(F)cc4[nH]ccc4c3F)ccc2F)[nH]1)c1cc(F)cc(CCC(=O)O)c1F] | 132A^{d} | Enantiomer 1 of 3-(3-(1-(2- (5-((4,6-difluoro-1H-indol-5- yl)oxy)-2-fluorophenyl)-1H- imidazol-5-yl)ethyl)-2,5- difluorophenyl)propanoic acid | (400 MHz, CD₃OD) δ 7.49 (dd, J = 6.0, 3.2 Hz, 1H), 7.30 (d, J = 3.2 Hz, 1H), 7.25-7.09 (m, 2H), 6.99-6.94 (m, 2H), 6.92- 6.85 (m, 1H), 6.76-6.70 (m, 1H), 6.55 (d, J = 3.2 Hz, 1H), 4.47 (q, J = 7.2 Hz, 1H), 2.94 (t, J = 7.6 Hz, 2H), 2.60 (t, J = 7.6 Hz, 2H), 1.58 (d, J = 7.2 Hz, 3H) ppm. | | 542 [M + H]⁺ |
| 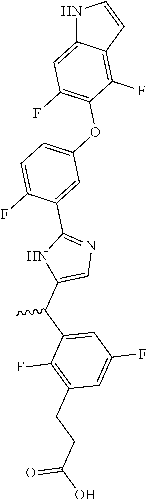 [SMILES: CC(c1cnc(-c2cc(Oc3c(F)cc4[nH]ccc4c3F)ccc2F)[nH]1)c1cc(F)cc(CCC(=O)O)c1F] | 132B^{d} | Enantiomer 2 of 3-(3-(1-(2- (5-((4,6-difluoro-1H-indol-5- yl)oxy)-2-fluorophenyl)-1H- imidazol-5-yl)ethyl)-2,5- difluorophenyl)propanoic acid | (400 MHz, CD₃OD) δ 7.49 (dd, J = 6.0, 3.2 Hz, 1H), 7.30 (d, J = 3.2 Hz, 1H), 7.24-7.09 (m, 2H), 7.01-6.93 (m, 2H), 6.93- 6.84 (m, 1H), 6.77-6.68 (m, 1H), 6.55 (d, J = 3.2 Hz, 1H), 4.47 (q, J = 7.2 Hz, 1H), 2.94 (t, J = 7.6 Hz, 2H), 2.60 (t, J = 7.6 Hz, 2H), 1.58 (d, J = 7.2 Hz, 3H) ppm. | | 542 [M + H]⁺ |
| 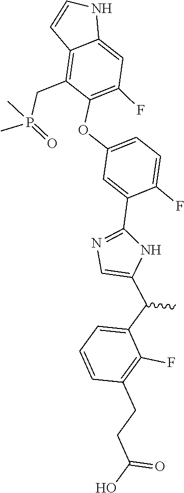 [SMILES: CC(c1cnc(-c2cc(Oc3c(F)cc4[nH]ccc4c3CP(C)(C)=O)ccc2F)[nH]1)c1cccc(CCC(=O)O)c1F] | 133A^{e, f} | Enantiomer 1 of 3-(3-(1-(2- (5-((4- ((dimethylphosphoryl)methyl)- 6-fluoro-1H-indol-5-yl)oxy)- 2-fluorophenyl)-1H-imidazol- 5-yl)ethyl)-2- fluorophenyl)propanoic acid | | 0.75 | 596 [M + H]⁺ |
| 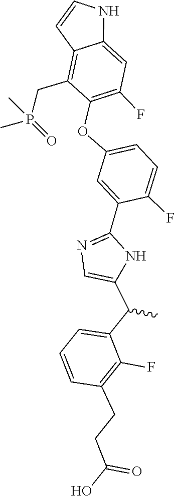 [SMILES: CC(c1cnc(-c2cc(Oc3c(F)cc4[nH]ccc4c3CP(C)(C)=O)ccc2F)[nH]1)c1cccc(CCC(=O)O)c1F] | 133B^{e, f} | Enantiomer 2 of 3-(3-(1-(2- (5-((4- ((dimethylphosphoryl)methyl)- 6-fluoro-1H-indol-5-yl)oxy)- 2-fluorophenyl)-1H-imidazol- 5-yl)ethyl)-2- fluorophenyl)propanoic acid | (400 MHz, CD₃OD) δ 7.43 (dd, J = 6.0, 2.8 Hz, 1H), 7.36 (d, J = 2.8 Hz, 1H), 7.25 (d, J = 10.8 Hz, 1H), 7.19 (t, J = 9.6 Hz, 1H), 7.16-7.12 (m, 1H), 7.02- 6.98 (m, 2H), 6.95 (s, 1H), 6.93-6.88 (m, 1H), 6.65 (d, J = 3.2 Hz, 1H), 4.49 (q, J = 6.8 Hz, 1H), 3.50 (d, J = 16.4 Hz, 2H), 2.96 (t, J = 7.6 Hz, 2H), 2.59 (t, J = 7.6 Hz, 2H), 1.59 (d, J = 7.2 Hz, 3H), 1.54 (d, J = 13.2 Hz, 6H) ppm. | 0.75 | 596 [M + H]⁺ |
| 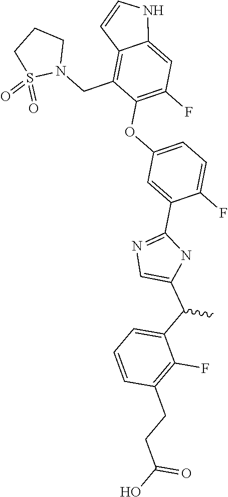 | 134A^{g} | Enantiomer 1 of 3-(3-(1-(2- (5-((4-((1,1- dioxidoisothiazolidin-2- yl)methyl)-6-fluoro-1H-indol- 5-yl)oxy)-2-fluorophenyl)- 1H-imidazol-5-yl)ethyl)-2- fluorophenyl)propanoic acid | (400 MHz, CD₃OD) δ 7.38 (dd, J = 6.0, 3.2 Hz, 1H), 7.34 (d, J = 3.6 Hz, 1H), 7.28 (d, J = 10.4 Hz, 1H), 7.20-7.10 (m, 2H), 7.00- 6.97 (m, 2H), 6.94-6.90 (m, 2H), 6.76 (d, J = 3.2 Hz, 1H), 4.47 (q, J = 7.2 Hz 1H), 4.36 (s, 2H), 3.10-3.04 (m, 4H), 2.94 (t, J = 7.6 Hz, 2H), 2.58 (t, J = 7.6 Hz, 2H), 2.13-2.06 (m, 2H), 1.58 (d, J = 7.2 Hz, 3H) ppm. | 0.85 | 639 [M + H]⁺ |
| 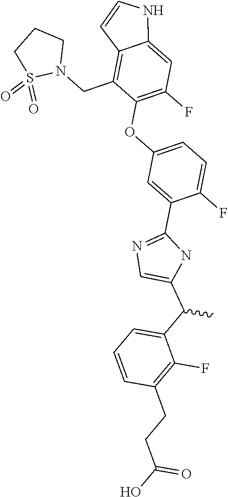 | 134B^{g} | Enantiomer 2 of 3-(3-(1-(2- (5-((4-((1,1- dioxidoisothiazolidin-2- yl)methyl)-6-fluoro-1H-indol- 5-yl)oxy)-2-fluorophenyl)- 1H-imidazol-5-yl)ethyl)-2- fluorophenyl)propanoic acid | (400 MHz, CD₃OD) δ 7.38 (q, J = 6.0, 3.2 Hz, 1H), 7.34 (d, J = 3.2 Hz, 1H), 7.28 (d, J = 10.4 Hz, 1H), 7.19-7.10 (m, 2H), 7.00-6.97 (m, 2H), 6.94-6.90 (m, 2H), 6.76 (d, J = 2.4 Hz, 1H), 4.47 (q, J = 7.2 Hz, 1H), 4.36 (s, 2H), 3.09-3.04 (m, 4H), 2.94 (t, J = 7.6 Hz, 2H), 2.58 (t, J = 7.6 Hz, 2H), 2.12- 2.05 (m, 2H), 1.58 (d, J = 7.2 Hz, 3H) ppm.. | 0.85 | 639 [M + H]⁺ |
| 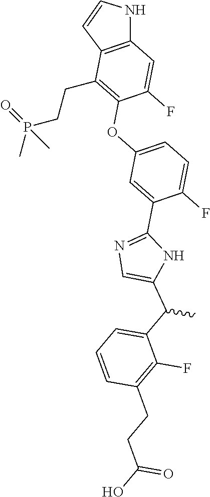 [SMILES: CC(c1cnc(-c2cc(Oc3c(F)cc4[nH]ccc4c3CCP(C)(C)=O)ccc2F)[nH]1)c1cccc(CCC(=O)O)c1F] | 135A^{h} | Enantiomer 1 of 3-(3-(1-(2- (5-((4-(2- (dimethylphosphoryl)ethyl)- 6-fluoro-1H-indol-5-yl)oxy)- 2-fluorophenyl)-1H-imidazol- 5-yl)ethyl)-2- fluorophenyl)propanoic acid | (400 MHz, CD₃OD) δ 7.44 (dd, J = 5.6, 2.8 Hz, 1H), 7.34 (d, J = 3.6 Hz, 1H), 7.24-7.17 (m, 2H), 7.16-7.12 (m, 1H), 7.06- 6.99 (m, 3H), 6.98-6.93 (m, 1H), 6.62 (d, J = 3.2 Hz, 1H), 4.50 (q, J = 7.6 Hz, 1H), 3.14 (dd, J = 8.4, 12.8 Hz, 2H), 2.96 (t, J = 7.6 Hz, 2H), 2.60 (t, J = 7.6 Hz, 2H), 2.17-2.09 (m, 2H), 1.61 (d, J = 7.2 Hz, 3H), 1.47 (d, J = 13.2 Hz, 6H) ppm. | 0.79 | 610 [M + H]⁺ |
| 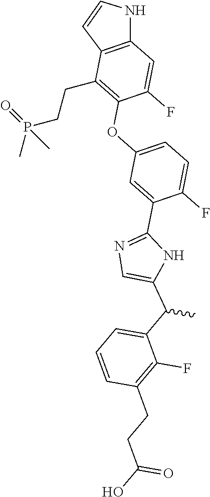 [SMILES: CC(c1cnc(-c2cc(Oc3c(F)cc4[nH]ccc4c3CCP(C)(C)=O)ccc2F)[nH]1)c1cccc(CCC(=O)O)c1F] | 135B^{h} | Enantiomer 2 of 3-(3-(1-(2- (5-((4-(2- (dimethylphosphoryl)ethyl)- 6-fluoro-1H-indol-5-yl)oxy)- 2-fluorophenyl)-1H-imidazol- 5-yl)ethyl)-2- fluorophenyl)propanoic acid | (400 MHz, CD₃OD) δ 7.44 (dd, J = 6.0, 3.2 Hz, 1H), 7.34 (d, J = 3.2 Hz, 1H), 7.24-7.17 (m, 2H), 7.16-7.12 (m, 1H), 7.06- 6.99 (m, 3H), 6.95 (dt, J = 9.2, 3.6 Hz, 1H), 6.62 (d, J = 3.2 Hz, 1H), 4.50 (q, J = 7.6 Hz, 1H), 3.13 (dd, J = 9.2, 17.2 Hz, 2H), 2.96 (t, J = 7.6 Hz, 2H), 2.60 (t, J = 7.6 Hz, 2H), 2.17- 2.08 (m, 2H), 1.61 (d, J = 7.2 Hz, 3H), 1.48 (d, J = 12.8 Hz, 6H) ppm. | 0.79 | 610 [M + H]⁺ |
| 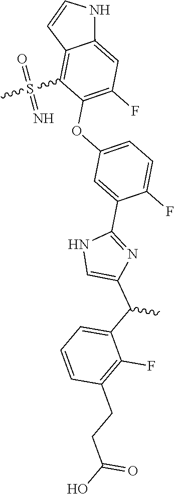 | 136Aⁱ | Enantiomer 1 of 3-(2-fluoro- 3-(1-(2-(2-fluoro-5-((6- fluoro-4-(S- methylsulfonimidoyl)-1H- indol-5-yl)oxy)phenyl)-1H- imidazol-4- yl)ethyl)phenyl)propanoic acid | | 0.72 | 583 [M + H]⁺ |
| 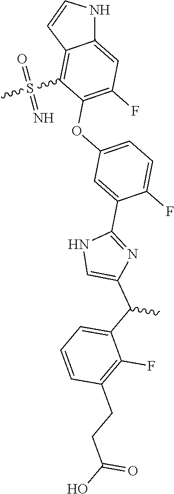 | 136Bⁱ | Enantiomer 2 of 3-(2-fluoro- 3-(1-(2-(2-fluoro-5-((6- fluoro-4-(S- methylsulfonimidoyl)-1H- indol-5-yl)oxy)phenyl)-1H- imidazol-4- yl)ethyl)phenyl)propanoic acid | | 0.72 | 583 [M + H]⁺ |
| 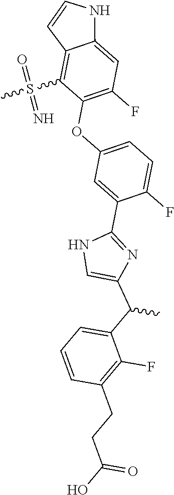 | 136Cⁱ | Enantiomer 3 of 3-(2-fluoro- 3-(1-(2-(2-fluoro-5-((6- fluoro-4-(S- methylsulfonimidoyl)-1H- indol-5-yl)oxy)phenyl)-1H- imidazol-4- yl)ethyl)phenyl)propanoic acid | | 0.72 | 583 [M + H]⁺ |
| 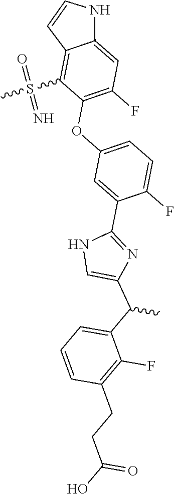 | 136Dⁱ | Enantiomer 4 of 3-(2-fluoro- 3-(1-(2-(2-fluoro-5-((6- fluoro-4-(S- methylsulfonimidoyl)-1H- indol-5-yl)oxy)phenyl)-1H- imidazol-4- yl)ethyl)phenyl)propanoic acid | | 0.72 | 583 [M + H]⁺ |
| 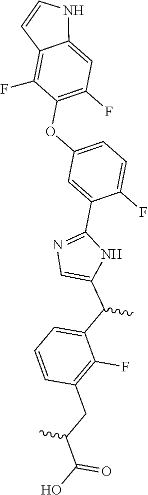 | 137A^{j} | Enantiomer 1 of 3-(3-(1-(2- (5-((4,6-difluoro-1H-indol-5- yl)oxy)-2-fluorophenyl)-1H- imidazol-5-yl)ethyl)-2- fluorophenyl)-2- methylpropanoic acid | | 0.97 | 538 [M + H]⁺ |
| 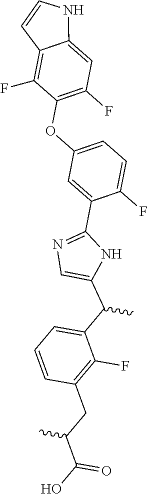 | 137B^{j} | Enantiomer 2 of 3-(3-(1-(2- (5-((4,6-difluoro-1H-indol-5- yl)oxy)-2-fluorophenyl)-1H- imidazol-5-yl)ethyl)-2- fluorophenyl)-2- methylpropanoic acid | | 0.97 | 538 [M + H]⁺ |
| 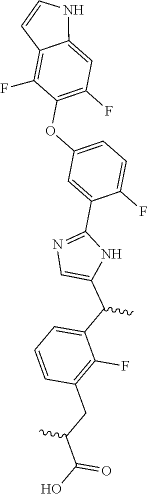 | 137C^{j} | Enantiomer 3 of 3-(3-(1-(2- (5-((4,6-difluoro-1H-indol-5- yl)oxy)-2-fluorophenyl)-1H- imidazol-5-yl)ethyl)-2- fluorophenyl)-2- methylpropanoic acid | | 0.97 | 538 [M + H]⁺ |
| 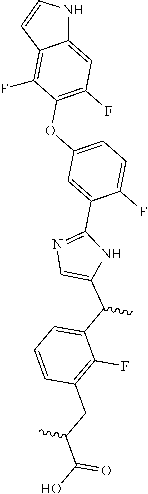 | 137D^{j} | Enantiomer 4 of 3-(3-(1-(2- (5-((4,6-difluoro-1H-indol-5- yl)oxy)-2-fluorophenyl)-1H- imidazol-5-yl)ethyl)-2- fluorophenyl)-2- methylpropanoic acid | | 0.98 | 538 [M + H]⁺ |

^{a}Chiral separation method was the same as that used for Example 128A.
^{b}Chiral separation method: unlike in the reference syntheses (Examples 128A/128B), enantiomer resolution was carried out at the final product stage (after ester hydrolysis). Thus, racemic carboxylic acid was subjected to chiral SFC: Waters/Thar Prep 80 system equipped with a 20 × 250 mm × 10 μm (R, R)Whelk-O1 column; mobile phase: 55:45 CO₂/methanol with 0.2% ammonia/methanol modifier; flow rate: 80 g/min; back pressure: 100 bar.
^{c}Chiral separation method: same as b except the mobile phase was changed to: 50:50 CO₂/ethanol with 0.2% ammonia/methanol modifier.
^{d}Chiral separation method: same as b except for mobile phase was changed to: 45:55 CO₂/methanol with 0.2% ammonia/methanol modifier.
^{e}Chiral separation method: same as b except the column and mobile phase were changed to: 20 × 250 mm × 10 μm (S, S)Whelk-O1 column; mobile phase: 35:65 CO₂/ methanol with 0.2% ammonia/methanol modifier.
^{f}The ester hydrolysis reaction, which affords the final product, employed sodium hydroxide (5 equivalents/room temperature) instead of the lithium hydroxide used in the reference syntheses (Examples 128A/128B).
^{g}Chiral separation method: same as b except the column and mobile phase were changed to: 20 × 250 mm × 10 μm (S, S)Whelk-O1 column; mobile phase: 35:65 CO₂/methanol with 0.2% methylethylamine modifier.
^{h}Chiral separation method: same as b except the column and mobile phase were changed to: 20 × 250 mm × 10 μm (R, R)Whelk-O1 column; mobile phase: 30:70 CO₂/ methanol with 0.2% methylethylamine modifier.
ⁱChiral separation method: enantiomer resolution was carried on the penultimate, ester intermediate. Three SFC separations were conducted to separate the four isomers. First SFC: the racemic ester was separated into two diastereomers (fraction 1 was comprised of enantiomer 1 and 2; fraction 2 was comprised of enantiomer 3 and 4). Separation conditions: Waters/Thar Prep 80 system equipped with a 20 × 250 mm × 10 μm CHIRALCEL OZ column; mobile phase: 30:70 CO₂/methanol; flow rate: 80 g/min; back pressure: 100 bar; column temperature: 35° C. Second SFC: fraction 1 was separated into enantiomer 1 and 2. Separation conditions: Waters/Thar Prep 80 system equipped with a 20 × 250 mm × 10 um (S, S)Whelk-O1 column; mobile phase: 50:50 CO₂/methanol with 0.2% ammonia/methanol modifier; flow rate: 80 g/min; back pressure: 100 bar; column temperature: 35° C. Third SFC: fraction 2 was separated into enantiomer 3 and 4. Separation conditions: Waters/Thar Prep 80 system equipped with a 20 × 250 mm × 10 μm CHIRAL ART Cellulose-SC column; mobile phase: 40:60 CO₂/methanol with 0.2% ammonia/methanol modifier; flow rate: 80 g/min; back pressure: 100 bar; column temperature: 35° C.
^{j}Chiral separation method: enantiomer resolution was carried on the penultimate, ester intermediate. One SFC and one chiral HPLC separations were conducted to separate the four isomers. First SFC: the racemic ester was separated into three diastereomers (fraction 1 was comprised of enantiomer 1 and 4; fraction 2 was comprised of enantiomer 2 and fraction 3 was comprised of enantiomer 3). Separation conditions: Waters/Thar Prep 80 system equipped with a 20 × 250 mm × 10 um AD column; mobile phase: 70:30 CO₂/methanol; flow rate: 80 g/min; back pressure: 100 bar; column temperature: 35° C. Second chiral separation: fraction 1 was separated into enantiomer 1 and enantiomer 4. Separation conditions: Prep HPLC system equipped with a 20 × 250 mm × 10 μm CHIRALPAK AD column; mobile phase: 85:15 Hexane/IPA (V/V); flow rate: 30 ml/min; column temperature: 35° C.

### Example 138

### 2-(2-Fluoro-3-(1-(2-(2-fluoro-5-((6-fluoro-4-methyl-1H-indol-5-yl)oxy)phenyl)-1H-imidazol-5-yl)ethyl)phenyl)cyclopropane-1-carboxylic acid (Eight Separated Stereoisomers)

### Absolute Configurations Unknown

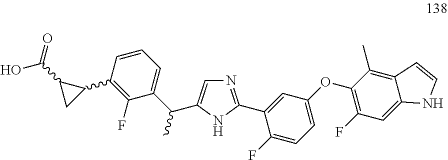 [SMILES: Cc1c(Oc2ccc(F)c(-c3ncc(C(C)c4cccc(C5CC5C(=O)O)c4F)[nH]3)c2)c(F)cc2[nH]ccc12]

### A. 5-(3-(5-(1-(3-Bromo-2-fluorophenyl)ethyl)-1H-imidazol-2-yl)-4-fluorophenoxy)-6-fluoro-4-methyl-1H-indole

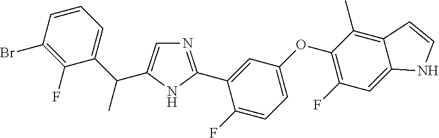 [SMILES: Cc1c(Oc2ccc(F)c(-c3ncc(C(C)c4cccc(Br)c4F)[nH]3)c2)c(F)cc2[nH]ccc12]

Exchanging 5-((4,6-difluoro-1H-indol-5-yl)oxy)-2-fluorobenzimidamide (Intermediate 24-9) for 2-fluoro-5-((6-fluoro-4-methyl-1H-indol-5-yl)oxy)benzimidamide (1.00 g, 3.32 mmol, Intermediate 24-10), and ethyl 3-(3-(3-chloro-2-oxopropyl)-2-fluorophenyl)propanoate for 3-(3-bromo-2-fluorophenyl)-1-chlorobutan-2-one (1.00 g, 3.58 mmol, Intermediate 21-14), the reaction conditions described for Step J of Example 100 were used to prepare the title compound as a yellow solid (1.40 g, 84%). MS: 526, 528 m/z [M+H]⁺.

### B. Ethyl 2-(2-fluoro-3-(1-(2-(2-fluoro-5-((6-fluoro-4-methyl-1H-indol-5-yl)oxy)phenyl)-1H-imidazol-5-yl)ethyl)phenyl)cyclopropane-1-carboxylate

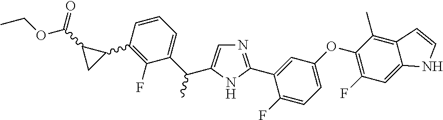 [SMILES: CCOC(=O)C1CC1c1cccc(C(C)c2cnc(-c3cc(Oc4c(F)cc5[nH]ccc5c4C)ccc3F)[nH]2)c1F]

To a stirred solution of step A product (1.46 g, 2.77 mmol), ethyl 2-(4,4,5,5-tetramethyl-1,3,2-dioxaborolan-2-yl)cyclopropanecarboxylate (1.37 g, 5.71 mmol) and potassium carbonate (2.00 g, 1.45 mmol) in 1,2-dimethoxyethane (20 mL) was added Pd(dppf)Cl₂ dichloromethane adduct (0.198 g, 0.242 mmol) and water (2 mL). The reaction was heated at 85° C. for four hours and then cooled to room temperature and diluted with ethyl acetate (80 mL). The mixture was washed with aqueous saturated sodium bicarbonate solution (1×100 mL) and brine (1×100 mL), dried over sodium sulfate and concentrated. The residue was purified by flash chromatography over silica (0-100% ethyl acetate in hexanes) to afford the title compound as a yellow solid (1.30 g, 84%). MS: 560 m/z [M+H]⁺.

### C. Separate Enantiomers (eight compounds) of ethyl 2-(2-fluoro-3-(1-(2-(2-fluoro-5-((6-fluoro-4-methyl-1H-indol-5-yl)oxy)phenyl)-1H-imidazol-5-yl)ethyl)phenyl)cyclopropane-1-carboxylate

The mixture of step B product isomers (1.25 g) was first separated by preparative HPLC to afford two fractions of the compound. Fraction one was a mixture of the two racemic diastereomers with cis-substitution on the cyclopropyl ring and fraction two was a mixture of the two racemic diastereomers with trans-substitution on the cyclopropyl ring (yield of each fraction: 0.450 g, 36% of the original mixture). The fraction one mixture of cis-cyclopropyl, step B isomers (0.450 g) was subjected to chiral SFC (Waters/Thar Prep 80 system equipped with a 20×250 mm×10 μm (R,R)Whelk-O1 column; mobile phase: 30:70 CO₂/methanol with 0.5% methylethylamine modifier; flow rate: 80 g/min; column temperature: 35° C.; back pressure: 100 bar) to a afford two fractions. Fraction one was a mixture of Enantiomers 1, 2 and 3 of the cis-cyclopropyl step B isomer mixture (0.300 g or 20% of the original mixture) and fraction two was pure Enantiomer 4 of the cis-cyclopropyl, step B isomer mixture (80 mg or 6.4% of the original mixture). The fraction one mixture was subjected to a second chiral SFC (same method as before except for the following changes: switched to a 20×250 mm×10 μm (S,S)Whelk-O1 column and a mobile phase modifier of 0.2% methylethylamine) to afford three fractions. The fractions were comprised of, in order, pure Enantiomer 1, pure Enantiomer 2 and pure Enantiomer 3 of the cis-cyclopropyl, step B isomer mixture (yields, respectively: 50 mg, 60 mg and 50 mg or 4.0%, 4.8% and 4.0% of the original mixture). Fraction two from the initial preparative HPLC separation (0.450 g; mixture of the four trans-cyclopropyl isomers of the step B product) was subjected to chiral SFC (Waters/Thar Prep 80 system equipped with a 20×250 mm×10 μm (R,R)Whelk-O1 column; mobile phase: 30:70 CO₂/methanol with 0.5% methylethylamine modifier; flow rate: 80 g/min; column temperature: 35° C.; back pressure: 100 bar) to afford three fractions. The fractions were comprised of, in order, a mixture of Enantiomers 1 and 2 of the trans-cyclopropyl, step B isomer mixture (120 mg or 9.6% of the original mixture), pure Enantiomer 3 of the trans-cyclopropyl, step B isomer mixture (70 mg or 5.6% of the original mixture) and pure Enantiomer 4 of the trans-cyclopropyl, step B isomer mixture (70 mg or 5.6% of the original mixture). The fraction one mixture was subjected to a second chiral SFC (same method as described for the second SFC on cis-cyclopropyl, step B isomer mixture) to afford two fractions. The fractions were comprised of pure Enantiomers 1 and pure Enantiomer 2 of the trans-cyclopropyl, step B isomer mixture (yield of each fraction: 40 mg or 3.2% of the original mixture).

### D. Separate Enantiomers of 2-(2-fluoro-3-(1-(2-(2-fluoro-5-((6-fluoro-4-methyl-1H-indol-5-yl)oxy)phenyl)-1H-imidazol-5-yl)ethyl)phenyl)cyclopropane-1-carboxylic acid

The eight enantiomers of step B product separated in step C were subjected, individually, to ester hydrolysis using a procedure analogous to that described in step K of the Example 100 synthesis. The eight stereoisomers of the title compound were all afforded as white solids. Characterizing data for the compounds is presented in the following table.

Example compounds comprising the eight stereoisomers of the title comound.

| | Ex. | | | LCMS | MS |
|---|---|---|---|---|---|
| Structure | No. | Name | ¹H NMR | Rₜ (min) | (m/z) |
| 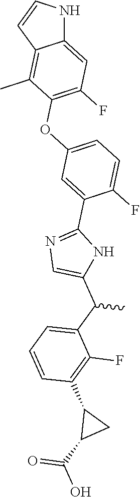 [SMILES: Cc1c(Oc2ccc(F)c(-c3ncc(C(C)c4cccc([C@@H]5C[C@@H]5C(=O)O)c4F)[nH]3)c2)c(F)cc2[nH]ccc12] | 138A | Enantiomer 1 of cis-2-(2- fluoro-3-(1-(2-(2-fluoro-5- ((6-fluoro-4-methyl-1H-indol- 5-yl)oxy)phenyl)-1H- imidazol-5- yl)ethyl)phenyl)cyclopropane- 1-carboxylic acid | (400 MHz, CD₃OD) δ 7.39 (m, 1H), 7.27 (d, J = 3.2 Hz, 1H), 7.20-6.79 (m, 7H), 6.51 (d, J = 3.2 Hz, 1H), 4.50 (q, J = 7.2 Hz, 1H) 2.66-2.57 (m, 1H), 2.38 (s, 3H), 1.92-1.76 (m, 1H), 1.67-1.47 (m, 4H), 1.45-1.35 (m, 1H) ppm. | 0.98 | 532 [M + H]⁺ |
| 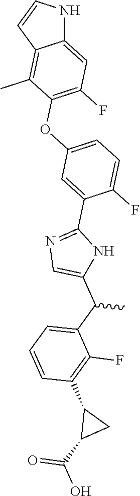 [SMILES: Cc1c(Oc2ccc(F)c(-c3ncc(C(C)c4cccc([C@@H]5C[C@@H]5C(=O)O)c4F)[nH]3)c2)c(F)cc2[nH]ccc12] | 138B | Enantiomer 2 of cis-2-(2- fluoro-3-(1-(2-(2-fluoro-5- ((6-fluoro-4-methyl-1H-indol- 5-yl)oxy)phenyl)-1H- imidazol-5- yl)ethyl)phenyl)cyclopropane- 1-carboxylic acid | (400 MHz, CD₃OD) δ 7.39 (m, 1H), 7.27 (d, J = 3.2 Hz, 1H), 7.20-6.79 (m, 7H), 6.51 (d, J = 3.2 Hz, 1H), 4.50 (q, J = 7.2 Hz, 1H) 2.66-2.57 (m, 1H), 2.38 (s, 3H), 1.92-1.76 (m, 1H), 1.65-1.44 (m, 4H), 1.40-1.32 (m, 1H) ppm. | 0.98 | 532 [M + H]⁺ |
| 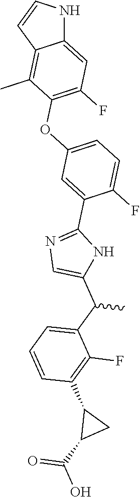 [SMILES: Cc1c(Oc2ccc(F)c(-c3ncc(C(C)c4cccc([C@@H]5C[C@@H]5C(=O)O)c4F)[nH]3)c2)c(F)cc2[nH]ccc12] | 138C | Enantiomer 3 of cis-2-(2- fluoro-3-(1-(2-(2-fluoro-5- ((6-fluoro-4-methyl-1H-indol- 5-yl)oxy)phenyl)-1H- imidazol-5- yl)ethyl)phenyl)cyclopropane- 1-carboxylic acid | (400 MHz, CD₃OD) δ 7.27 (m, 1H), 7.14 (d, J = 3.6 Hz, 1H), 7.09-6.88 (m, 7H), 6.51 (d, J = 3.2 Hz, 1H), 4.47 (q, J = 7.2 Hz, 1H) 2.52-2.56 (m, 1H), 2.38 (s, 3H), 2.11-2.17 (m, 1H), 1.56 (m, 3H), 1.43-1.37 (m, 2H) ppm. | 0.96 | 532 [M + H]⁺ |
| 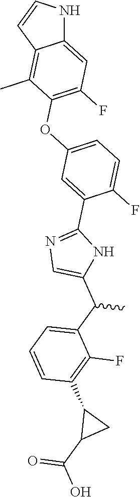 [SMILES: Cc1c(Oc2ccc(F)c(-c3ncc(C(C)c4cccc([C@@H]5CC5C(=O)O)c4F)[nH]3)c2)c(F)cc2[nH]ccc12] | 138D | Enantiomer 4 of cis-2-(2- fluoro-3-(1-(2-(2-fluoro-5- ((6-fluoro-4-methyl-1H-indol- 5-yl)oxy)phenyl)-1H- imidazol-5- yl)ethyl)phenyl)cyclopropane- 1-carboxylic acid | (400 MHz, CD₃OD) δ 7.27 (m, 1H), 7.14 (d, J = 3.6 Hz, 1H), 7.09-6.88 (m, 7H), 6.51 (d, J = 3.2 Hz, 1H), 4.47 (q, J = 7.2 Hz, 1H) 2.52-2.56 (m, 1H), 2.38 (s, 3H), 2.11-2.17 (m, 1H), 1.56 (m, 3H), 1.43-1.37 (m, 2H) ppm. | 0.96 | 532 [M + H]⁺ |
| 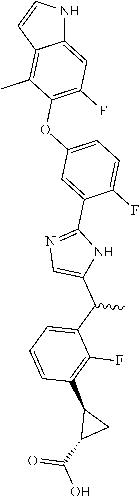 [SMILES: Cc1c(Oc2ccc(F)c(-c3ncc(C(C)c4cccc([C@H]5C[C@@H]5C(=O)O)c4F)[nH]3)c2)c(F)cc2[nH]ccc12] | 139E | Enantiomer 1 of trans-2-(2- fluoro-3-(1-(2-(2-fluoro-5- ((6-fluoro-4-methyl-1H-indol- 5-yl)oxy)phenyl)-1H- imidazol-5- yl)ethyl)phenyl)cyclopropane- 1-carboxylic acid | (400 MHz, CD₃OD) δ 7.39 (m, 1H), 7.27 (d, J = 3.2 Hz, 1H), 7.20-6.79 (m, 7H), 6.51 (d, J = 3.2 Hz, 1H), 4.50 (q, J = 7.2 Hz, 1H) 2.66-2.57 (m, 1H), 2.38 (s, 3H), 1.92-1.76 (m, 1H), 1.67-1.47 (m, 4H), 1.45-1.35 (m, 1H) ppm. | 0.98 | 532 [M + H]⁺ |
| 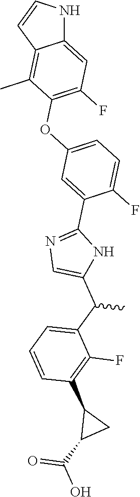 [SMILES: Cc1c(Oc2ccc(F)c(-c3ncc(C(C)c4cccc([C@H]5C[C@@H]5C(=O)O)c4F)[nH]3)c2)c(F)cc2[nH]ccc12] | 139F | Enantiomer 2 of trans-2-(2- fluoro-3-(1-(2-(2-fluoro-5- ((6-fluoro-4-methyl-1H-indol- 5-yl)oxy)phenyl)-1H- imidazol-5- yl)ethyl)phenyl)cyclopropane- 1-carboxylic acid | (400 MHz, CD₃OD) δ 7.39 (m, 1H), 7.27 (d, J = 3.2 Hz, 1H), 7.20-6.79 (m, 7H), 6.51 (d, J = 3.2 Hz, 1H), 4.50 (q, J = 7.2 Hz, 1H) 2.66-2.57 (m, 1H), 2.38 (s, 3H), 1.92-1.76 (m, 1H), 1.67-1.47 (m, 4H), 1.45-1.35 (m, 1H) ppm. | 0.98 | 532 [M + H]⁺ |
| 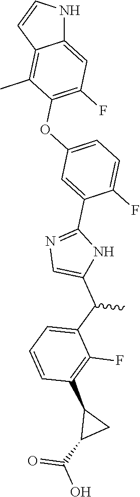 [SMILES: Cc1c(Oc2ccc(F)c(-c3ncc(C(C)c4cccc([C@H]5C[C@@H]5C(=O)O)c4F)[nH]3)c2)c(F)cc2[nH]ccc12] | 139G | Enantiomer 3 of trans-2-(2- fluoro-3-(1-(2-(2-fluoro-5- ((6-fluoro-4-methyl-1H-indol- 5-yl)oxy)phenyl)-1H- imidazol-5- yl)ethyl)phenyl)cyclopropane- 1-carboxylic acid | (400 MHz, CD₃OD) δ 7.39 (m, 1H), 7.27 (d, J = 3.2 Hz, 1H), 7.20-6.83 (m, 7H), 6.51 (d, J = 3.2 Hz, 1H), 4.51 (q, J = 7.2 Hz, 1H), 2.62-2.54 (m, 1H), 2.38 (s, 3H), 1.85-1.81 (m, 1H), 1.61-1.49 (m, 4H), 1.41-1.37 (m, 1H) ppm. | 0.98 | 532 [M + H]⁺ |
| 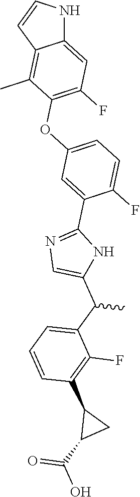 [SMILES: Cc1c(Oc2ccc(F)c(-c3ncc(C(C)c4cccc([C@H]5C[C@@H]5C(=O)O)c4F)[nH]3)c2)c(F)cc2[nH]ccc12] | 139H | Enantiomer 4 of trans-2-(2- fluoro-3-(1-(2-(2-fluoro-5- ((6-fluoro-4-methyl-1H-indol- 5-yl)oxy)phenyl)-1H- imidazol-5- yl)ethyl)phenyl)cyclopropane- 1-carboxylic acid | (400 MHz, CD₃OD) δ 7.39 (m, 1H), 7.27 (d, J = 3.2 Hz, 1H), 7.20-6.83 (m, 7H), 6.51 (d, J = 3.2 Hz, 1H), 4.51 (q, J = 7.2 Hz, 1H), 2.62-2.54 (m, 1H), 2.38 (s, 3H), 1.85-1.81 (m, 1H), 1.61-1.49 (m, 4H), 1.41-1.37 (m, 1H) ppm. | 0.98 | 532 [M + H]⁺ |

### Example 140

### 3-(3-((2-(2-Fluoro-5-((6-fluoro-4-(methylcarbamoyl)-1H-indol-5-yl)oxy)phenyl)-1H-imidazol-5-yl)methyl)phenyl)propanoic acid

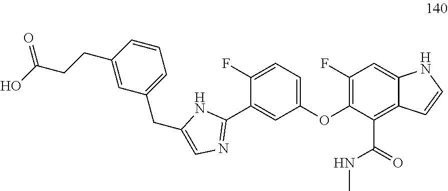 [SMILES: CNC(=O)c1c(Oc2ccc(F)c(-c3ncc(Cc4cccc(CCC(=O)O)c4)[nH]3)c2)c(F)cc2[nH]ccc12]

### A. 5-(3-Cyano-4-fluorophenoxy)-6-fluoro-1-tosyl-1H-indole-4-carboxylic acid

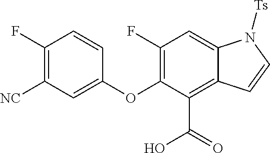 [SMILES: Cc1ccc(S(=O)(=O)n2ccc3c(C(=O)O)c(Oc4ccc(F)c(C#N)c4)c(F)cc32)cc1]

A screw-top reaction flask was loaded with 2-fluoro-5-((6-fluoro-4-formyl-1-tosyl-1H-indol-5-yl)oxy)benzonitrile (Intermediate 9; 2.20 g, 4.87 mmol), 3:3:1 THF/tert-butanol/water (140 mL), sodium chlorite (3.52 g, 38.9 mmol), potassium phosphate monobasic (5.29 g, 38.9 mmol) and 2-methyl-2-butene (5.1 g, 73 mmol). The vessel was sealed and the reaction was stirred overnight at room temperature. After this time, the mixture was extracted with ethyl acetate (3×60 mL). The combined extracts were dried over sodium sulfate and concentrated. The residue was purified by flash chromatography over silica (0-10% methanol in dichloromethane) to afford the title compound as white solid (1.71 g, 75%). MS: 469 m/z [M+H]⁺.

### B. 5-(3-Cyano-4-fluorophenoxy)-6-fluoro-N-methyl-1-tosyl-1H-indole-4-carboxamide

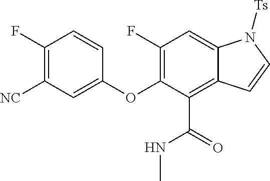 [SMILES: CNC(=O)c1c(Oc2ccc(F)c(C#N)c2)c(F)cc2c1ccn2S(=O)(=O)c1ccc(C)cc1]

To a stirred and cooled (0° C.) solution of step A product (0.700 g, 1.49 mmol), methylamine hydrochloride (2.03 g, 30.0 mmol) and HATU (1.14 g, 3.00 mmol) in DMF (15 mL) was added, dropwise over five minutes, triethylamine (5.2 mL, 37 mmol). The reaction was allowed to warm to room temperature, stirred for two hours and then poured into ice water (50 mL). The precipitate was collected by filtration and purified by flash chromatography over silica (0-3% methanol in dichloromethane). The title compound was afforded as yellow solid (0.680 g, 95%). MS: 482 m/z [M+H]⁺.

### C. 5-(3-Cyano-4-fluorophenoxy)-6-fluoro-N-methyl-1H-indole-4-carboxamide

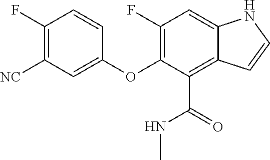 [SMILES: CNC(=O)c1c(Oc2ccc(F)c(C#N)c2)c(F)cc2[nH]ccc12]

To a stirred solution of step B product (0.680 g, 1.41 mmol) in methanol (6 mL) was added potassium carbonate (85.0 mg, 0.615 mmol). The reaction was heated for 30 minutes at 80° C. in a microwave reactor then cooled to room temperature and diluted with water (20 mL). The mixture was extracted with dichloromethane (3×20 mL) and the combined extracts were washed with brine (1×40 mL), dried over magnesium sulfate and concentrated. The residue was purified by flash chromatography over silica (0-3% methanol in dichloromethane) to afford the title compound as a white solid (0.310 g, 67%). MS: 328 m/z [M+H]⁺.

### D. 5-(3-Carbamimidoyl-4-fluorophenoxy)-6-fluoro-N-methyl-1H-indole-4-carboxamide

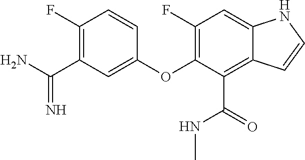 [SMILES: CNC(=O)c1c(Oc2ccc(F)c(C(=N)N)c2)c(F)cc2[nH]ccc12]

To a stirred and cooled (0° C.) solution of step C product (0.290 g, 0.886 mmol) in THF (10 mL) was added a 1.0 M solution of lithium bis(trimethylsilyl)amide in THF (7.1 mL, 7.1 mmol). The mixture was allowed to warm to room temperature and stirred overnight. After this time, the reaction was quenched with the additional of methanol (10 mL) and then concentrated. The residue was purified by flash chromatography over silica (15% methanol in dichloromethane with 1% concentrated ammonium hydroxide) to afford the title compound as a yellow solid (0.260 g, 85%). MS: 345 m/z [M+H]⁺.

### E. Methyl 3-(3-((2-(2-fluoro-5-((6-fluoro-4-(methylcarbamoyl)-1H-indol-5-yl)oxy)phenyl)-1H-imidazol-5-yl)methyl)phenyl)propanoate

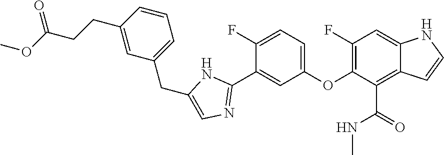 [SMILES: CNC(=O)c1c(Oc2ccc(F)c(-c3ncc(Cc4cccc(CCC(=O)OC)c4)[nH]3)c2)c(F)cc2[nH]ccc12]

A stirred mixture of step D product (0.200 g. 0.581 mmol), methyl 3-(3-(3-bromo-2-oxopropyl)phenyl)propanoate (Intermediate 21; 0.190 g, 0.635 mmol) and potassium carbonate (0.160 g, 1.16 mmol) in DMF (10 mL) was heated overnight at 75° C. After this time, the reaction was concentrated and the residue was purified by flash chromatography over silica (3% methanol in dichloromethane with 1% concentrated ammonium hydroxide). The title compound was afforded as a yellow solid (0.100 g, 32%). MS: 545 m/z [M+H]⁺.

### F. Example 140, 3-(3-((2-(2-Fluoro-5-((6-fluoro-4-(methylcarbamoyl)-1H-indol-5-yl)oxy)phenyl)-1H-imidazol-5-yl)methyl)phenyl)propanoic acid

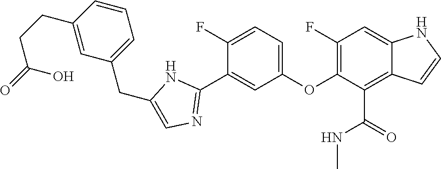 [SMILES: CNC(=O)c1c(Oc2ccc(F)c(-c3ncc(Cc4cccc(CCC(=O)O)c4)[nH]3)c2)c(F)cc2[nH]ccc12]

To stirred solution of step E product (0.100 g, 0.184 mmol) in 2:1 THF/water (15 mL) was added lithium hydroxide (44.0 mg, 1.84 mmol). The reaction was stirred at room temperature for three hours and then concentrated. The residue was dissolved in water (2 mL) and, with stirring, this solution was acidified (˜pH 3) with the dropwise addition of 1.0 N hydrochloric acid. The resulting precipitate was collected by suction filtration, rinsed with additional water (2×1 mL) and vacuum oven dried to afford the title compound as a white solid (90 mg, 92%). ¹H NMR (400 MHz, CD₃OD) δ 7.44-7.39 (m, 3H), 7.35-7.22 (m, 4H), 7.17-7.16 (m, 2H), 7.12 (d, J=7.2 Hz, 1H), 6.59 (d, J=2.4 Hz, 1H), 4.08 (s, 2H), 2.91 (t, J=7.2 Hz, 2H), 2.87 (s, 3H), 2.59 (t, J=7.2 Hz, 2H) ppm. MS: 531 m/z [M+H]⁺.

### Example 141

### 3-(2-(Aminomethyl)-3-((2-(5-((4,6-difluoro-1H-indol-5-yl)oxy)-2-fluorophenyl)-1H-imidazol-5-yl)methyl)phenyl)propanoic acid

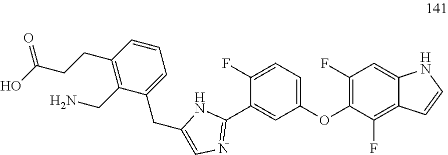 [SMILES: NCc1c(CCC(=O)O)cccc1Cc1cnc(-c2cc(Oc3c(F)cc4[nH]ccc4c3F)ccc2F)[nH]1]

A stirred suspension of Example 122 (0.100 g, 0.194 mmol) and Raney-Ni (50 mg) in a 4 M solution of ammonia in methanol (10 mL) was cycled between vacuum and a nitrogen atmosphere three times. After evacuating a final time, the vessel was backfilled with hydrogen (via balloon). The reaction was stirred overnight at room temperature and then filtered through a pad of Celite. The filtering agent was rinsed with additional methanol (˜30 mL) and the combined filtrate was concentrated. The residue was purified by preparative HPLC to afford the title compound as a white solid (14 mg, 14%) as a white solid. ¹H NMR (400 MHz, CD₃OD) δ 7.46 (dd, J=6.0, 3.2 Hz, 1H), 7.31 (d, J=3.2 Hz, 1H), 7.29-7.24 (m, 2H), 7.19-7.14 (m, 3H), 7.03 (s, 1H), 6.87-6.84 (m, 1H), 6.56 (d, J=2.8 Hz, 1H), 4.46 (s, 2H), 4.07 (s, 2H), 3.00 (t, J=7.6 Hz, 2H), 2.66 (t, J=7.6 Hz, 2H) ppm. MS: 521 m/z [M+H]⁺.

### Example 142

### 3-(3-(4-(2-(5-((4,6-Difluoro-1H-indol-5-yl)oxy)-2-fluorophenyl)-1H-imidazol-5-yl)-2,6-dimethyltetrahydro-2H-pyran-4-yl)phenyl)propanoic acid

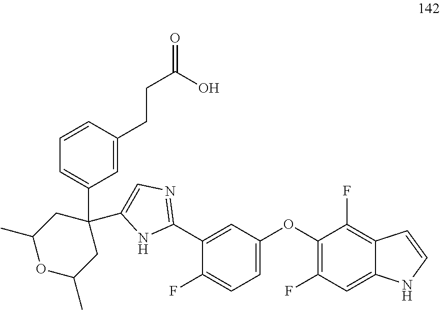 [SMILES: CC1CC(c2cccc(CCC(=O)O)c2)(c2cnc(-c3cc(Oc4c(F)cc5[nH]ccc5c4F)ccc3F)[nH]2)CC(C)O1]

### A. Diethyl 2,2′-oxydipropionate

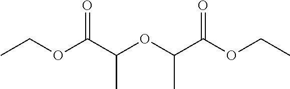

To a stirred and cooled (0° C.) solution of ethyl lactate (19.4 mL, 169 mmol) in THF (150 mL) was added, portionwise over five minutes, a 60% dispersion of sodium hydride in mineral oil (10.00 g, 250.0 mmol). The frothy mixture was stirred at 0° C. for 30 minutes before adding, dropwise over 10 minutes, ethyl 2-bromopropanoate (19.5 mL, 150 mmol). The reaction was then brought to 80° C. and maintained at this temperature overnight. After this time, the mixture was cooled to room temperature, poured into cooled (0° C.) 4 N hydrochloric acid (100 mL) and extracted with ethyl acetate (3×100 mL). The combined extracts were washed with brine (1×100 mL), dried over sodium sulfate and concentrated. The residue was purified by flash chromatography over silica (10% ethyl acetate in petroleum ether) to afford the title compound as a yellow oil (10.01 g, 31%). ¹H NMR (400 MHz, DMSO-d₆) δ 4.13-4.07 (m, 6H), 1.30 (d, J=6.8 Hz, 6H), 1.20 (d, J=7.2 Hz, 6H) ppm.

### B. 2,2′-Oxybis(propan-1-ol)

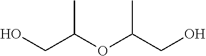

To a stirred solution of step A product (5.00 g, 22.9 mmol) in THF (200 mL) was added, portionwise, lithium aluminum hydride (2.00 g, 52.7 mmol). Following the addition, the reaction was warmed to 30° C. After two hours at this temperature, the mixture quenched with the careful, portionwise addition of solid sodium sulfate decahydrate (added until the precipitated salts appeared white). The solids were filtered off and rinsed with THF (3×100 mL). The combined filtrate was concentrated to afford the crude title compound as a colorless oil (2.03 g, 66

### C. Oxybis(propane-2,1-diyl)bis(4-methylbenzenesulfonate)

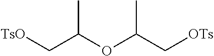 [SMILES: Cc1ccc(S(=O)(=O)OCC(C)OC(C)COS(=O)(=O)c2ccc(C)cc2)cc1]

To a stirred solution of step B product (1.40 g, 10.4 mmol) in dichloromethane (50 mL) was added pyridine (3.4 mL, 42 mmol) and TsCl (4.37 g, 22.9 mmol). The reaction was allowed to proceed for two hours at room temperature and then concentrated and partitioned between water (50 mL) and dichloromethane (100 mL). The organic layer was combined with additional extracts (dichloromethane, 2×100 mL), washed with brine (1×100 mL), dried over magnesium sulfate and concentrated. The residue was purified by flash chromatography over silica (10% ethyl acetate in petroleum ether) to afford the title compound as a yellow oil (2.30 g, 50%). ¹H NMR (400 MHz, DMSO-d₆) δ 7.77 (d, J=8.0 Hz, 4H), 7.47 (d, J=8.0 Hz, 4H), 3.92 (dd, J=10.0, 2.8 Hz, 2H), 3.78 (dd, J=10.4, 6.4 Hz, 2H), 3.68-3.64 (m, 2H), 2.42 (s, 6H), 0.89 (d, J=6.4 Hz, 6H) ppm.

### D. 4-(3-Bromophenyl)-2,6-dimethyltetrahydro-2H-pyran-4-carbonitrile

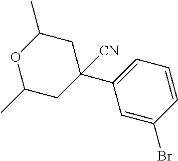 [SMILES: CC1CC(C#N)(c2cccc(Br)c2)CC(C)O1]

To a stirred solution of 2-(3-bromophenyl)acetonitrile (1 g, 5.10 mmol) in DMF (10 mL) was slowly added dry sodium hydride (0.610 g, 25.4 mmol). The frothy mixture was stirred for five minutes before adding, dropwise, step C product (2.26 g, 5.10 mmol). The reaction mixture was heated overnight at 70° C. and then quenched with the addition of water (dropwise at first; total, 100 mL). The resulting suspension was extracted with ethyl acetate (3×50 mL) and the combined extracts were washed with water (1×100 mL) and brine (1×100 mL). The solution was then dried over magnesium sulfate and concentrated to afford a residue which was subjected to flash chromatography over silica (5-20% ethyl acetate in cyclohexane). The title compound was obtained as a yellow solid (1.01 g, 67%). MS: 294, 296 m/z [M+H]⁺.

### E. 4-(3-Bromophenyl)-2,6-dimethyltetrahydro-2H-pyran-4-carboxylic acid

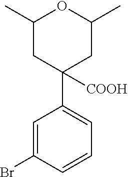 [SMILES: CC1CC(C(=O)O)(c2cccc(Br)c2)CC(C)O1]

To a stirred suspension of step D product (1.00 g, 3.40 mmol) in 5:1 ethanol/water (12 mL) was added potassium hydroxide (3.83 g, 68.2 mmol). The reaction was heated overnight at 100° C. and then cooled to room temperature and concentrated to remove the organic solvent. The residue was diluted with water (50 mL) and this solution was acidified (pH 2-3) by the dropwise addition of concentrated hydrochloric acid. The resulting suspension was extracted with ethyl acetate (3×50 mL) and the combined organic layers were washed with brine (1×100 mL), dried over magnesium sulfate and concentrated. The crude title compound, which was used without purification, was obtained as a yellow oil (1.00 g, 94%). ¹H NMR (400 MHz, DMSO-d₆) δ 12.75 (br s, 1H), 7.52 (s, 1H), 7.49 (d, J=8.0 Hz, 1H), 7.42 (d, J=8.0 Hz, 1H), 7.33 (t, J=8.0 Hz, 1H), 4.15-4.11 (m, 1H), 3.97-3.94 (m, 1H), 2.49 (d, J=13.6 Hz, 1H), 2.38 (d, J=13.6 Hz, 1H), 2.03 (dd, J=9.6, 5.6 Hz, 2H), 1.19 (d, J=6.0 Hz, 3H), 1.06 (d, J=6.0 Hz, 3H) ppm. MS: 313, 315 m/z [M+H]⁺.

### F. (E)-4-(3-(3-Methoxy-3-oxoprop-1-en-1-yl)phenyl)-2,6-dimethyltetrahydro-2H-pyran-4-carboxylic acid

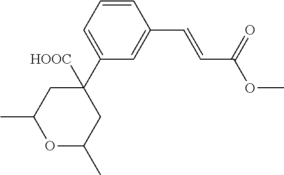

To a stirred solution of step E product (1.00 g, 3.19 mmol) in DMF (15 mL) was added methyl acrylate (862 μL, 9.57 mmol), (o-Tol)₃P (0.290 g, 0.953 mmol), triethylamine (1.34 mL, 9.62 mmol) and Pd(OAc)₂ (0.140 g, 0.623 mmol). The reaction was heated overnight at 100° C. and then cooled to room temperature and diluted with water (100 mL). The resulting suspension was acidified (˜pH 3) by the dropwise addition of 2.0 N hydrochloric acid and extracted with ethyl acetate (3×50 mL). The combined extracts were dried over magnesium sulfate and concentrated, affording a residue which was subjected to flash chromatography silica (40% ethyl acetate in cyclohexane). The title compound was obtained as a yellow oil (0.914 g, 90%). MS: 319 m/z [M+H]⁺.

### G. 4-(3-(3-Methoxy-3-oxopropyl)phenyl)-2,6-dimethyltetrahydro-2H-pyran-4-carboxylic acid

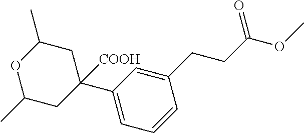

Into a stirred suspension of step F product (0.900 g, 2.83 mmol) and 10% Pd/C (0.200 g) in ethyl acetate (100 mL) was bubbled with hydrogen for two hours. The mixture was then suction filtered through a pad of Celite, which was subsequently washed with ethyl acetate (˜100 mL). The combined filtrate was concentrated to afford the crude title compound, which was used without purification, as a colorless oil (0.816 g, 90%). MS: 343 m/z [M+Na]⁺.

### H. Methyl 3-(3-(4-(2-bromoacetyl)-2,6-dimethyltetrahydro-2H-pyran-4-yl)phenyl)propanoate

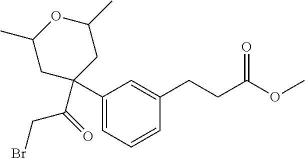

To a stirred and cooled (0° C.) solution of step G product (0.844 g, 2.63 mmol) in dichloromethane (30 mL) was added oxalyl chloride (1.4 mL, 16 mmol) followed by one drop of DMF (gas evolution observed). The reaction was allowed to warm to room temperature and then stirred for one hour before concentrating. The obtained residue was co-evaporated with heptane (20 mL) several times to remove trace oxalyl chloride and taken up in 1:1 tetrahydrofuran/acetonitrile (50 mL). To this stirred and cooled (0° C.) solution of crude acid chloride was added, dropwise over five minutes, a 2.0 M solution of trimethylsilyldiazomethane in THF (6.3 mL, 12.6 mmol). The mixture was allowed to warm to room temperature and stirred overnight. After this time, the mixture was returned to 0° C. and treated with, dropwise over 10 minutes, a 33% solution of hydrogen bromide in acetic acid (2.2 mL, 12 mmol; vigorous gas evolution observed). The reaction was allowed to warm to room temperature and then stirred for another 30 minutes before partitioning between brine (75 mL) and ethyl acetate (50 mL). The organic layer was combined with additional extracts (ethyl acetate, 2×50 mL), washed with brine (1×100 mL) and dried over magnesium sulfate. The solution was concentrated to afford a residue which was purified by flash chromatography over silica (5-20% ethyl acetate in cyclohexane) to afford the title compound as colorless oil (0.670 g, 64%) as a colorless oil. MS: 397, 399 m/z [M+H]⁺.

### I. 5-((4,6-Difluoro-1H-indol-5-yl)oxy)-2-fluorobenzimidamide

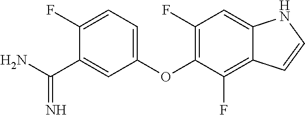 [SMILES: N=C(N)c1cc(Oc2c(F)cc3[nH]ccc3c2F)ccc1F]

To a stirred and cooled (0° C.) solution of 5-((4,6-difluoro-1H-indol-5-yl)oxy)-2-fluorobenzonitrile (Product of Step H, Example 100; 0.440 g, 1.53 mmol) in THF (10 mL) was added a 1.0 M solution of lithium bis(trimethylsilyl)amide in THF (6.1 mL, 6.1 mmol). The reaction was allowed to warm to room temperature overnight and stirred overnight. After this time, the mixture was partitioned between ethyl acetate (100 mL) and saturated aqueous ammonium chloride solution (50 mL). The organic layer was washed with brine (2×10 mL), dried over sodium sulfate and concentrated. Crude title compound, which was used without purification, was afforded as a pale amber solid (0.457 g, 100%). MS: 306 m/z [M+H]⁺.

### J. Methyl 3-(3-(4-(2-(5-((4,6-difluoro-1H-indol-5-yl)oxy)-2-fluorophenyl)-1H-imidazol-5-yl)-2,6-dimethyltetrahydro-2H-pyran-4-yl)phenyl)propanoate

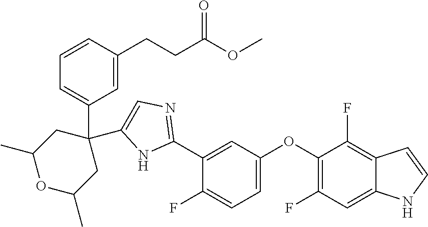 [SMILES: COC(=O)CCc1cccc(C2(c3cnc(-c4cc(Oc5c(F)cc6[nH]ccc6c5F)ccc4F)[nH]3)CC(C)OC(C)C2)c1]

A mixture of step H product (0.197 g, 0.496 mmol), step I product (Product of Step I, Example 100, 0.150 g, 0.491 mmol) and potassium carbonate (0.135 g, 0.977 mmol) in acetonitrile (10 mL) was heated overnight at 80° C. The reaction mixture was then cooled to room temperature and partitioned between ethyl acetate (100 mL) and water (50 mL). The organic layer was washed with additional water (1×50 mL) and brine (1×50 mL), dried over sodium sulfate and concentrated. The obtained residue was purified by flash chromatography over silica (40% ethyl acetate in petroleum ether) to provide the title compound as a yellow oil (0.160 g, 54%).

### K. Example 142, 3-(3-(4-(2-(5-((4,6-Difluoro-1H-indol-5-yl)oxy)-2-fluorophenyl)-1H-imidazol-5-yl)-2,6-dimethyltetrahydro-2H-pyran-4-yl)phenyl)propanoic acid

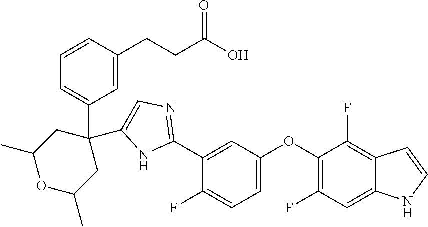 [SMILES: CC1CC(c2cccc(CCC(=O)O)c2)(c2cnc(-c3cc(Oc4c(F)cc5[nH]ccc5c4F)ccc3F)[nH]2)CC(C)O1]

To a stirred solution of step I product (0.150 g, 0.248 mmol) in 1:1 methanol/water (10 mL) was added lithium hydroxide (0.112 g, 4.68 mmol). The reaction was stirred over a weekend at room temperature and then concentrated to remove organic solvent. The remaining aqueous mixture was diluted with water (3 mL) and acidified (to pH 2-3) by the dropwise addition of 1.0 N hydrochloric acid. The resulting suspension was extracted with ethyl acetate (3×30 mL) and the combined extracts were washed with brine (1×30 mL) and dried over magnesium sulfate. The solution was then concentrated to afford a residue which was purified by preparative HPLC. The title compound was obtained as a white solid (50 mg, 34%). ¹H NMR (400 MHz, CD₃OD) δ 7.43-7.40 (D, 1H), 7.31 (d, J=3.2 Hz, 1H), 7.24 (s, 1H), 7.23-7.12 (m, 4H), 7.04 (d, J=6.8 Hz, 1H), 6.95-6.90 (m, 2H), 6.56 (d, J=3.2 Hz H, 1H), 4.23-4.18 (m, 2H), 2.87 (t, J=7.6 Hz, 2H), 2.60-2.37 (m, 5H), 1.99-1.93 (m, 1H), 1.08 (d, J=6.4 Hz, 3H), 1.03 (d, J=6.4 Hz, 3H) ppm. MS: 590 m/z [M+H]⁺.

The following Examples compounds were prepared by synthetic routes analogous to that described for Example 142.

| | Ex. | | | LCMS | LCMS |
|---|---|---|---|---|---|
| Structure | No. | Name | ¹H NMR | Rₜ (min) | (m/z) |
| 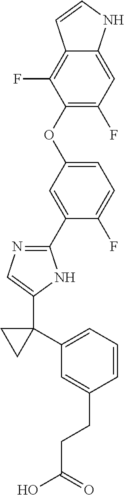 [SMILES: O=C(O)CCc1cccc(C2(c3cnc(-c4cc(Oc5c(F)cc6[nH]ccc6c5F)ccc4F)[nH]3)CC2)c1] | 143 | 3-(3-(1-(2-(5-((4,6-difluoro- 1H-indol-5-yl)oxy)-2- fluorophenyl)-1H-imidazol-5- yl)cyclopropyl)phenyl)propanoic acid | (400 MHz, CD₃OD) δ 7.49 (dd, J = 6.0, 3.2 Hz, 1H), 7.30 (d, J = 2.8 Hz, 1H), 7.21-7.10 (m, 5H), 7.06 (d, J = 7.6 Hz, 1H), 6.94 (dt, J = 9.6, 3.6 Hz, 1H), 6.78 (s, 1H), 6.56 (d, J = 3.2 Hz, 1H), 2.88 (t, J = 7.6 Hz, 2H), 2.56 (t, J = 7.6 Hz, 2H), 1.37- 1.34 (m, 2H), 1.24-1.21 (m, 2H) ppm. | 0.97 | 518 [M + H]⁺ |
| 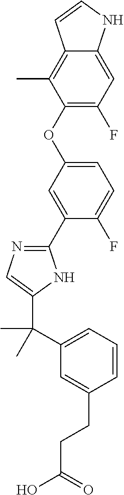 [SMILES: Cc1c(Oc2ccc(F)c(-c3ncc(C(C)(C)c4cccc(CCC(=O)O)c4)[nH]3)c2)c(F)cc2[nH]ccc12] | 144 | 3-(3-(2-(2-(2-fluoro-5-((6- fluoro-4-methyl-1H-indol-5- yl)oxy)phenyl)-1H-imidazol- 5-yl)propan-2- yl)phenyl)propanoic acid | (400 MHz, CD3OD) 8 7.35-7.33 (m, 1H), 7.28 (d, J = 3.2 Hz, 1H), 7.24- 7.18 (m, 3H), 7.07-7.03 (m, 4 H), 6.93-6.89 (m, 1H), 6.52 (d, J = 3.2 Hz, 1H), 2.88 (t, J = 7.6 Hz, 2H), 2.56 (t, J = 7.6 Hz, 2H), 2.38 (s, 3H), 1.72 (s, 6H) ppm. | 0.93 | 516 [M + H]⁺ |
| 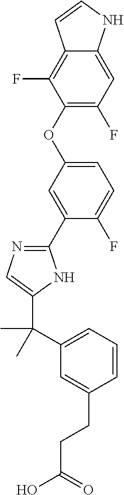 [SMILES: CC(C)(c1cccc(CCC(=O)O)c1)c1cnc(-c2cc(Oc3c(F)cc4[nH]ccc4c3F)ccc2F)[nH]1] | 145 | 3-(3-(2-(2-(5-((4,6-difluoro- 1H-indol-5-yl)oxy)-2- fluorophenyl)-1H-imidazol-5- yl)propan-2- yl)phenyl)propanoic acid | (400 MHz, CD₃OD) δ 7.43 (q, J = 3.2 Hz, 1H), 7.30 (d, J = 3.2 Hz, 1H), 7.25-7.00 (m, 8H), 6.55 (d, J = 3.6 Hz, 1H), 2.88 (t, J = 7.6 Hz, 2H), 2.56 (t, J = 7.6 Hz, 2H), 1.71 (s, 6H) ppm. | 0.92 | 520 [M + H]⁺ |
| 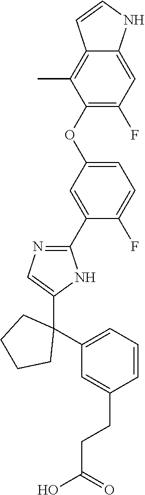 [SMILES: Cc1c(Oc2ccc(F)c(-c3ncc(C4(c5cccc(CCC(=O)O)c5)CCCC4)[nH]3)c2)c(F)cc2[nH]ccc12] | 146 | 3-(3-(1-(2-(2-fluoro-5-((6- fluoro-4-methyl-1H-indol-5- yl)oxy)phenyl)-1H-imidazol- 5- yl)cyclopentyl)phenyl)propanoic acid | (400 MHz CD₃OD) δ 7.38 (s, 1H), 7.33 (dd, J = 6.0, 2.8 Hz, 1H), 7.29 (d, J = 4.0 Hz, 1H), 7.26-7.22 (m, 3H), 7.19-7.16 (m, 1H), 7.13-7.08 (m, 2H), 6.99 (dt, J = 9.6, 3.2 Hz, 1H), 6.53 (d, J = 3.2 Hz, 1H), 2.89 (t, J = 7.6 Hz, 2H), 2.57 (t, J = 7.6 Hz, 2H), 2.44-2.39 (m, 2H), 2.38 (s, 3H), 2.32-2.26 (m, 2H), 1.84-1.77 (m, 4H) ppm. | 1.00 | 542 [M + H]⁺ |
| 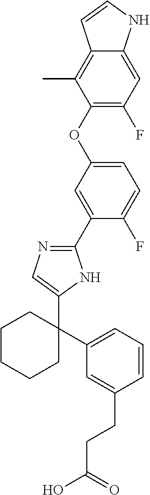 [SMILES: Cc1c(Oc2ccc(F)c(-c3ncc(C4(c5cccc(CCC(=O)O)c5)CCCCC4)[nH]3)c2)c(F)cc2[nH]ccc12] | 147 | 3-(3-(1-(2-(2-fluoro-5-((6- fluoro-4-methyl-1H-indol-5- yl)oxy)phenyl)-1H-imidazol- 4- yl)cyclohexyl)phenyl)propanoic acid | (400 MHz, CD₃OD) δ 7.36 (dd, J = 6.0, 2.8 Hz, 1H), 7.27 (d, J = 3.2 Hz, 1H), 7.23-7.09 (m, 5H), 7.04 (d, J = 6.8 Hz, 1H), 6.86 (s, 1H), 6.80 (dt, J = 9.2, 3.6 Hz, 1H), 6.52 (d, J = 3.2 Hz, 1H), 2.88 (t, J = 7.6 Hz, 2H), 2.55 (t, J = 7.6 Hz, 2H), 2.39 (s, 3H), 2.36-2.33 (m, 2H), 2.22-2.16 (m, 2H), 1.63-1.50 (m, 6H) ppm. | 1.08 | 556 [M + H]⁺ |
| 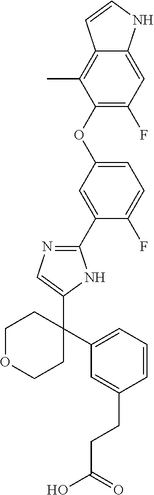 [SMILES: Cc1c(Oc2ccc(F)c(-c3ncc(C4(c5cccc(CCC(=O)O)c5)CCOCC4)[nH]3)c2)c(F)cc2[nH]ccc12] | 148 | 3-(3-(4-(2-(2-fluoro-5-((6- fluoro-4-methyl-1H-indol-5- yl)oxy)phenyl)-1H-imidazol- 5-yl)tetrahydro-2H-pyran-4- yl)phenyl)propanoic acid | (400 MHz, CD₃OD) δ 7.39 (dd, J = 6.4, 3.2 Hz, 1H), 7.27 (d, J = 3.2 Hz, 1H), 7.25-7.03 (m, 6H), 6.98 (s, 1H), 6.82-6.77 (m, 1H), 6.52 (dd, J = 3.2, 0.8 Hz, 1H), 3.84-3.68 (m, 4H), 2.88 (t, J = 7.6 Hz, 2H), 2.54 (t, J = 7.6 Hz, 2H), 2.52-2.46 (m, 2H), 2.39 (s, 3H), 2.36-2.27 (m, 2H) ppm. | 0.92 | 558 [M + H]⁺ |
| 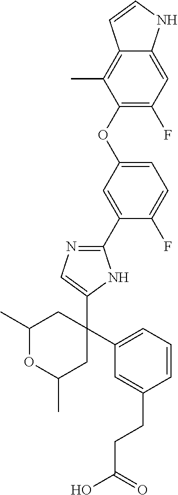 [SMILES: Cc1c(Oc2ccc(F)c(-c3ncc(C4(c5cccc(CCC(=O)O)c5)CC(C)OC(C)C4)[nH]3)c2)c(F)cc2[nH]ccc12] | 149 | 3-(3-(4-(2-(2-fluoro-5-((6- fluoro-4-methyl-1H-indol-5- yl)oxy)phenyl)-1H-imidazol- 5-yl)-2,6-dimethyltetrahydro- 2H-pyran-4- yl)phenyl)propanoic acid | (400 MHz, CD₃OD) δ 7.33 (dd, J = 6.0, 3.2 Hz, 1H), 7.26 (d, J = 3.2 Hz, 1H), 7.23 (s, 1H), 7.20-7.03 (m, 5H), 6.90 (s, 1H), 6.82 (dt, J = 9.2, 3.6 Hz, 1H), 6.51 (d, J = 3.2 Hz, 1H), 4.23-4.16 (m, 2H), 2.87 (t, J = 7.6 Hz, 2H), 2.59-2.40 (m, 5H), 2.38 (s, 3H), 1.97 (dd, J = 8.4, 13.6 Hz, 1H), 1.08 (d, J = 6.4 Hz, 3H), 1.04 (d, J = 6.4 Hz, 3H) ppm | 0.99 | 586 [M + H]⁺ |
| 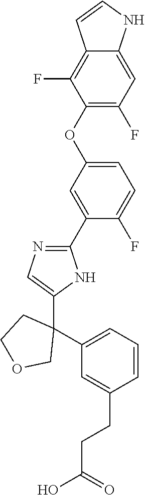 [SMILES: O=C(O)CCc1cccc(C2(c3cnc(-c4cc(Oc5c(F)cc6[nH]ccc6c5F)ccc4F)[nH]3)CCOC2)c1] | 150 | 3-(3-(3-(2-(5-((4,6-difluoro- 1H-indol-5-yl)oxy)-2- fluorophenyl)-1H-imidazol-5- yl)tetrahydrofuran-3- yl)phenyl)propanoic acid | (400 MHz, CD₃OD) δ 7.70 (s, 1H), 7.45-7.41 (m, 1H), 7.38 (d, J = 9.6 Hz, 1H), 7.34 (d, J = 2.8 Hz, 1H), 7.30 (d, J = 7.6 Hz, 1H), 7.25-7.16 (m, 5H), 6.56 (d, J = 2.8 Hz, 1H), 4.41 (d, J = 8.8 Hz, 1H), 4.18 (d, J = 8.8 Hz, 1H), 4.10-4.02 (m, 2H), 2.91 (t, J = 7.6 Hz, 2H), 2.75-2.68 (m, 2H), 2.59 (t, J = 7.6 Hz, 2H) ppm. | 0.92 | 548 [M + H]⁺ |
| 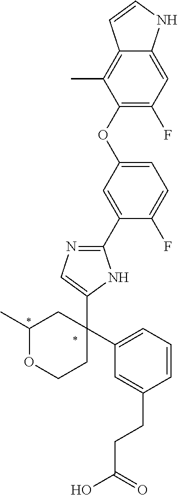 [SMILES: Cc1c(Oc2ccc(F)c(-c3ncc(C4(c5cccc(CCC(=O)O)c5)CCOC(C)C4)[nH]3)c2)c(F)cc2[nH]ccc12] | 151 | Isomer 1 of 3-(3-(4-(2-(2- fluoro-5-((6-fluoro-4-methyl- 1H-indol-5-yl)oxy)phenyl)- 1H-imidazol-5-yl)-2- methyltetrahydro-2H-pyran- 4-yl)phenyl)propanoic acid (racemic, cis or trans, relative stereochemistry not determined, isomers separated by flash chromatography over silica) | (400 MHz, CD₃OD) δ 7.36-7.31 (m, 4H), 7.29-7.27 (m, 1H), 7.14-7.09 (m, 3H), 6.81-6.78 (m, 1H), 6.53- 6.52 (m, 2H), 3.88 (d, J = 10.4 Hz, 1H), 3.58 (t, J = 12.2 Hz, 2H), 2.93 (t, J = 7.2 Hz, 2H), 2.69 (d, J = 13.4 Hz, 1H), 2.60 (s, 3H), 2.39 (s, 3H), 2.33-2.26 (t, J = 12.8 Hz, 1H), 1.98 (t, J = 13.2 Hz, 1H), 1.20 (d, J = 6.0 Hz, 3H) ppm. | 0.95 | 572 [M + H]⁺ |
| 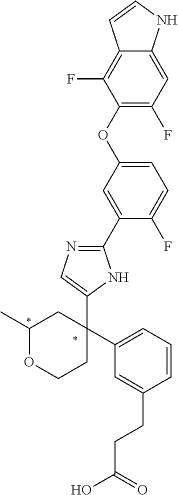 [SMILES: CC1CC(c2cccc(CCC(=O)O)c2)(c2cnc(-c3cc(Oc4c(F)cc5[nH]ccc5c4F)ccc3F)[nH]2)CCO1] | 152 | Isomer 2 of 3-(3-((2S)-4-(2- (5-((4,6-difluoro-1H-indol-5- yl)oxy)-2-fluorophenyl)-1H- imidazol-5-yl)-2- methyltetrahydro-2H-pyran- 4-yl)phenyl)propanoic acid (racemic, cis or trans, relative stereochemistry not determined, isomers separated by flash chromatography over silica) | (400 MHz, CD₃OD) δ 7.45 (s, 1H), 7.36-7.26 (m, 4H), 7.18-7.17 (m, 3H), 6.93-6.91 (m, 1H), 6.54 (d, J = 16.0 Hz, 2H), 3.88 (d, J = 11.2 Hz, 1H), 3.58 (t, J = 11.2 Hz, 2H), 2.93 (t, J = 7.6 Hz, 2H), 2.70-2.53 (m, 4H), 2.30 (t, J = 12.2 Hz, 1H), 1.98 (t, J = 12.4 Hz, 1H), 1.20 (d, J = 5.6 Hz, 3H) ppm. | 0.94 | 576 [M + H]⁺ |
| 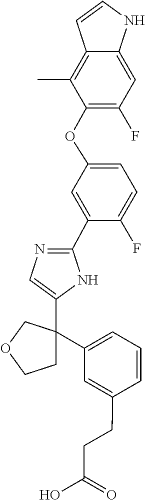 [SMILES: Cc1c(Oc2ccc(F)c(-c3ncc(C4(c5cccc(CCC(=O)O)c5)CCOC4)[nH]3)c2)c(F)cc2[nH]ccc12] | 153 | 3-(3-(3-(2-(2-fluoro-5-((6- fluoro-4-methyl-1H-indol-5- yl)oxy)phenyl)-1H-imidazol- 5-yl)tetrahydrofuran-3- yl)phenyl)propanoic acid | (400 MHz, CD₃OD) δ 7.39 (dd, J = 6.0, 3.2 Hz, 1H), 7.27 (d, J = 3.2 Hz, 1H), 7.26-7.19 (m, 2H), 7.16-7.08 (m, 4H), 7.05 (s, 1H), 6.81 (dt, J = 8.8, 3.2 Hz, 1H), 6.51 (d, J = 2.8 Hz, 1H), 4.39 (d, J = 8.0 Hz, 1H), 4.22 (d, J = 8.4 Hz, 1H), 4.02-3.96 (m, 2H), 2.88 (t, J = 7.6 Hz, 2H), 2.83- 2.77 (m, 1H), 2.57-2.50 (m, 3H), 2.38 (s, 3H) ppm. | 0.93 | 544 [M + H]⁺ |
| 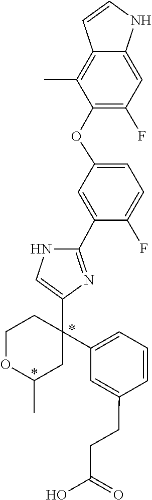 [SMILES: Cc1c(Oc2ccc(F)c(-c3nc(C4(c5cccc(CCC(=O)O)c5)CCOC(C)C4)c[nH]3)c2)c(F)cc2[nH]ccc12] | 154 | Isomer 2 of 3-(3-(4-(2-(2- fluoro-5-((6-fluoro-4-methyl- 1H-indol-5-yl)oxy)phenyl)- 1H-imidazol-4-yl)-2- methyltetrahydro-2H-pyran- 4-yl)phenyl)propanoic acid (racemic, cis or trans, relative stereochemistry not determined, isomers separated by flash chromatography over silica) | (400 MHz, CD₃OD) δ 7.38 (dd, J = 5.6, 2.8 Hz, 1H), 7.27 (d, J = 3.2 Hz, 1H), 7.19-7.15 (m, 2H), 7.15-7.14 (m, 2H), 7.12-7.09 (m, 2H), 7.02 (d, J = 7.6 Hz, 1H), 6.80 (dt, J = 8.8, 3.6 Hz, 1H), 6.52 (d, J = 3.2 Hz, 1H), 3.93 (dd, J = 11.6, 3.2 Hz, 1H), 3.73-3.67 (m, 2H), 2.86 (t, J = 7.6 Hz, 2H), 2.62 (d, J = 13.6 Hz, 1H), 2.54-2.49 (m, 3H), 2.38 (s, 3H), 2.12 (td, J = 12.8, 4.4 Hz, 1H), 1.76 (dd, J = 13.2, 11.2 Hz, 1H), 1.19 (d, J = 6.4 Hz, 3H) ppm | 0.96 | 572 [M + H]⁺ |
| 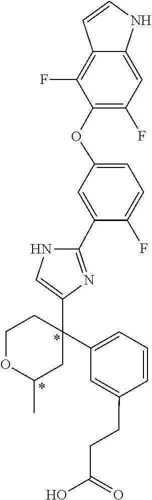 [SMILES: CC1CC(c2cccc(CCC(=O)O)c2)(c2c[nH]c(-c3cc(Oc4c(F)cc5[nH]ccc5c4F)ccc3F)n2)CCO1] | 155 | Isomer 1 of 3-(3-((2S)-4-(2- (5-((4,6-difluoro-1H-indol-5- yl)oxy)-2-fluorophenyl)-1H- imidazol-5-yl)-2- methyltetrahydro-2H-pyran- 4-yl)phenyl)propanoic acid (racemic, cis or trans, relative stereochemistry not determined, isomers separated by flash chromatography over silica) | (400 MHz, CD₃OD) δ 7.47-7.45 (m, 1H), 7.30 (d, J = 3.2 Hz, 1H), 7.20- 7.08 (m, 6H), 7.02-7.01 (m, 1H), 6.95-6.91 (m, 1H), 6.55 (d, J = 3.2 Hz, 1H), 3.94-3.91 (m, 1H), 3.73-3.67 (m, 2H), 2.86 (t, J = 7.6 Hz, 2H), 2.62 (d, J = 13.6 Hz, 1H), 2.54-2.49 (m, 3H), 2.16-2.08 (m, 1H), 1.79-1.73 (m, 1H), 1.19 (d, J = 6.0 Hz, 3H) ppm. | 0.95 | 576 [M + H]⁺ |
| 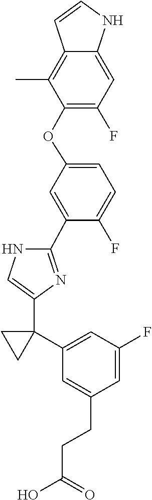 [SMILES: Cc1c(Oc2ccc(F)c(-c3nc(C4(c5cc(F)cc(CCC(=O)O)c5)CC4)c[nH]3)c2)c(F)cc2[nH]ccc12] | 156 | 3-(3-fluoro-5-(1-(2-(2-fluoro- 5-((6-fluoro-4-methyl-1H- indol-5-yl)oxy)phenyl)-1H- imidazol-5- yl)cyclopropyl)phenyl)propanoic acid | (400 MHz, CD₃OD) δ 7.41 (dd, J = 6.0, 3.2 Hz, 1H), 7.27 (d, J = 3.2 Hz, 1H), 7.18-7.08 (m, 2H), 6.96 (s, 1H), 6.86 (s, 1H), 6.85-6.84 (m, 1H), 6.81 (dd, J = 9.6, 1.6 Hz, 2H), 6.52 (dd, J = 3.2, 0.8 Hz, 1H), 2.88 (t, J = 7.6 Hz, 2H), 2.57 (t, J = 7.6 Hz, 2H), 2.40 (s, 3H), 1.39-1.36 (m, 2H), 1.26-1.23 (m, 2H) ppm. | 0.99 | 532 [M + H]⁺ |
| 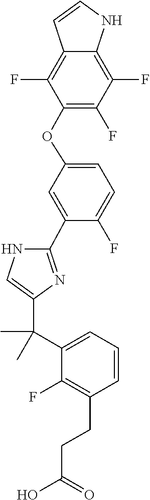 [SMILES: CC(C)(c1c[nH]c(-c2cc(Oc3c(F)c(F)c4[nH]ccc4c3F)ccc2F)n1)c1cccc(CCC(=O)O)c1F] | 157 | 3-(2-fluoro-3-(2-(2-(2-fluoro- 5-((4,6,7-trifluoro-1H-indol- 5-yl)oxy)phenyl)-1H- imidazol-5-yl)propan-2- yl)phenyl)propanoic acid | (400 MHz, CD₃OD) δ 7.47 (dd, J = 5.6, 3.2 Hz, 1H), 7.37 (d, J = 3.2 Hz, 1H), 7.36-6.95 (m, 6H), 6.62 (t, J = 2.8 Hz, 1H), 2.89 (t, J = 7.6 Hz, 2H), 2.53 (t, J = 7.6 Hz, 2H), 1.78 (s, 6H) ppm. | 0.95 | 556 [M + H]⁺ |
| 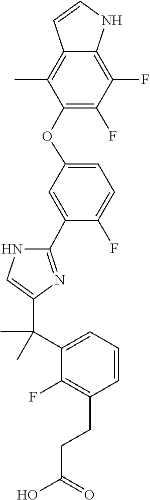 [SMILES: Cc1c(Oc2ccc(F)c(-c3nc(C(C)(C)c4cccc(CCC(=O)O)c4F)c[nH]3)c2)c(F)c(F)c2[nH]ccc12] | 158 | 3-(3-(2-(2-(5-((6,7-difluoro-4- methyl-1H-indol-5-yl)oxy)-2- fluorophenyl)-1H-imidazol-4- yl)propan-2-yl)-2- fluorophenyl)propanoic acid | (400 MHz, CD₃OD) δ 7.38-7.35 (m, 2H), 7.27-7.17 (m, 4H), 7.07 (t, J = 8.0 Hz, 1H), 6.99-6.95 (m, 1H), 6.59 (t, J = 3.6 Hz, 1H), 2.89 (t, J = 7.6 Hz, 2H), 2.54 (t, J = 7.6 Hz, 2H), 2.34 (s, 3H), 1.77 (s, 6H) ppm. | 0.97 | 552 [M + H]⁺ |
| 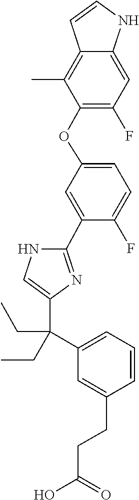 [SMILES: CCC(CC)(c1cccc(CCC(=O)O)c1)c1c[nH]c(-c2cc(Oc3c(F)cc4[nH]ccc4c3C)ccc2F)n1] | 159 | 3-(3-(3-(2-(2-fluoro-5-((6- fluoro-4-methyl-1H-indol-5- yl)oxy)phenyl)-1H-imidazol- 5-yl)pentan-3- yl)phenyl)propanoic acid | (400 MHz, CD₃OD) δ 7.30-7.27 (m, 1H), 7.26 (d, J = 3.2 Hz, 1H), 7.16 (t, J = 8.0 Hz, 1H), 7.14 (s, 1H), 7.13-7.01 (m, 5H), 6.81 (dt, J = 8.8, 3.2 Hz, 1H), 6.51 (d, J = 3.2 Hz, 1H), 2.84 (t, J = 7.6 Hz, 2H), 2.42 (t, J = 7.6 Hz, 2H), 2.37 (s, 3H), 2.16- 2.09 (m, 4H), 0.69 (t, J = 7.2 Hz, 6H) ppm. | 1.04 | 544 [M + H]⁺ |
| 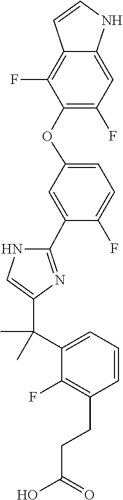 [SMILES: CC(C)(c1c[nH]c(-c2cc(Oc3c(F)cc4[nH]ccc4c3F)ccc2F)n1)c1cccc(CCC(=O)O)c1F] | 160 | 3-(3-(2-(2-(5-((4,6-difluoro- 1H-indol-5-yl)oxy)-2- fluorophenyl)-1H-imidazol-5- yl)propan-2-yl)-2- fluorophenyl)propanoic acid | (400 MHz, CD₃OD) δ 7.46 (dd, J = 6.0, 3.2 Hz, 1H), 7.30 (d, J = 3.2 Hz, 1H), 7.24-7.08 (m, 4H), 7.07-6.90 (m, 3H), 6.55 (dd, J = 3.2, 0.8 Hz, 1H), 2.90 (t, J = 7.6 Hz, 2H), 2.54 (t, J = 7.6 Hz, 2H), 1.76 (s, 6H) ppm. | 0.93 | 538 [M + H]⁺ |
| 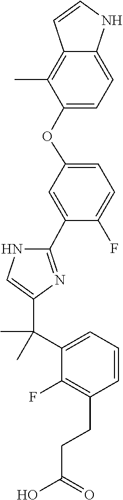 [SMILES: Cc1c(Oc2ccc(F)c(-c3nc(C(C)(C)c4cccc(CCC(=O)O)c4F)c[nH]3)c2)ccc2[nH]ccc12] | 161 | 3-(2-fluoro-3-(2-(2-(2-fluoro- 5-((4-methyl-1H-indol-5- yl)oxy)phenyl)-1H-imidazol- 5-yl)propan-2- yl)phenyl)propanoic acid | (400 MHz, CD₃OD) δ 7.37 (dd, J = 6.0, 3.2 Hz, 1H), 7.28 (d, J = 3.2 Hz, 1H), 7.25 (d, J = 8.6 Hz, 1H), 7.18- 7.08 (m, 3H), 7.01 (m, 1H), 6.93 (s, 1H), 6.84-6.76 (m, 2H), 6.52-6.49 (m, 1H), 2.90 (t, J = 7.6 Hz, 2H), 2.53 (t, J = 7.6 Hz, 2H), 2.35 (s, 3H), 1.75 (s, 6H) ppm. | 0.92 | 516 [M+H]+ |
| 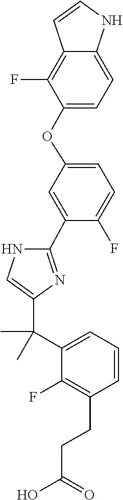 [SMILES: CC(C)(c1c[nH]c(-c2cc(Oc3ccc4[nH]ccc4c3F)ccc2F)n1)c1cccc(CCC(=O)O)c1F] | 162 | 3-(2-fluoro-3-(2-(2-(2-fluoro- 5-((4-fluoro-1H-indol-5- yl)oxy)phenyl)-1H-imidazol- 5-yl)propan-2- yl)phenyl)propanoic acid | (400 MHz, CD₃OD) δ 7.44 (dd, J = 6.1, 3.1 Hz, 1H), 7.30 (d, J = 3.2 Hz, 1H), 7.25-7.18 (m, 1H), 7.18-7.06 (m, 3H), 7.01 (t, J = 7.6 Hz, 1H), 6.97-6.85 (m, 3H), 6.55 (dd, J = 3.2, 0.8 Hz, 1H), 2.90 (t, J = 7.7 Hz, 2H), 2.53 (t, J = 7.7 Hz, 2H), 1.75 (s, 6H) ppm. | 0.91 | 520 [M + H]⁺ |
| 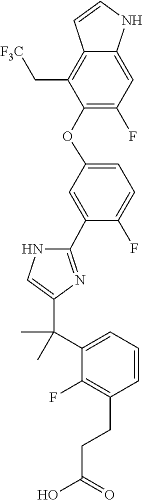 [SMILES: CC(C)(c1c[nH]c(-c2cc(Oc3c(F)cc4[nH]ccc4c3CC(F)(F)F)ccc2F)n1)c1cccc(CCC(=O)O)c1F] | 163 | 3-(2-fluoro-3-(2-(2-(2-fluoro- 5-((6-fluoro-4-(2,2,2- trifluoroethyl)-1H-indol-5- yl)oxy)phenyl)-1H-imidazol- 5-yl)propan-2- yl)phenyl)propanoic acid | (400 MHz, CD₃OD) δ 7.42-7.40 (m, 1H), 7.36 (d, J = 3.2 Hz, 1H), 7.30 (d, J = 10.8 Hz, 1H), 7.23-7.15 (m, 3H), 7.10 (s, 1H), 7.05 (t, J = 7.6 Hz, 1H), 6.94-6.90 (m, 1H), 6.58 (d, J = 3.2 Hz, 1H), 3.75 (q, J = 10.8 Hz, 2H), 2.89 (t, J = 7.6 Hz, 2H), 2.54 (t, J = 7.6 Hz, 2H), 1.77 (s, 6H) ppm. | 1.00 | 602 [M + H]⁺ |
| 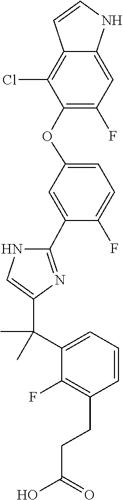 [SMILES: CC(C)(c1c[nH]c(-c2cc(Oc3c(F)cc4[nH]ccc4c3Cl)ccc2F)n1)c1cccc(CCC(=O)O)c1F] | 164 | 3-(3-(2-(2-(5-((4-chloro-6- fluoro-1H-indol-5-yl)oxy)-2- fluorophenyl)-1H-imidazol-4- yl)propan-2-yl)-2- fluorophenyl)propanoic acid | (400 MHz, CD3OD). 7.41 (dd, J = 6.0, 3.2 Hz, 1H), 7.35 (d, J = 3.6 Hz, 1H), 7.25 (dd, J = 10.0, 0.8 Hz, 1H), 7.17-7.06 (m, 3H), 6.99 (t, J = 7.6 Hz, 1H), 6.92 (s, 1H), 6.84 (dt, J = 9.2, 3.2 Hz, 1H), 6.54 (dd, J = 3.2, 0.4 Hz, 1H), 2.88 (t, J = 7.6 Hz, 2H), 2.52 (t, J = 7.6 Hz, 2H) , 1.73 (s, 6H) ppm. | 0.96 | 554 [M + H]⁺ |
| 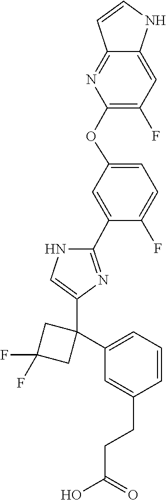 [SMILES: O=C(O)CCc1cccc(C2(c3c[nH]c(-c4cc(Oc5nc6cc[nH]c6cc5F)ccc4F)n3)CC(F)(F)C2)c1] | 165 | 3-(3-(3,3-difluoro-1-(2-(2- fluoro-5-((6-fluoro-1H- pyrrolo[3,2-b]pyridin-5- yl)oxy)phenyl)-1H-imidazol- 5- yl)cyclobutyl)phenyl)propanoic acid | (400 MHz, CD₃OD) δ 7.78 (dd, J = 10.4, 0.8 Hz, 1H), 7.69 (dd, J = 6.0, 3.2 Hz, 1H), 7.51 (d, J = 3.2 Hz, 1H), 7.29-7.20 (m, 4H), 7.16-7.10 (m, 2H), 6.91 (s, 1H), 6.44 (dd, J = 3.2, 0.8 Hz, 1H), 3.43-3.34 (m, 2H), 3.32-3.15 (m, 2H), 2.91 (t, J = 7.6 Hz, 2H), 2.59 (t, J = 7.6 Hz, 2H) ppm. | 0.94 | 551 [M + H]⁺ |
| 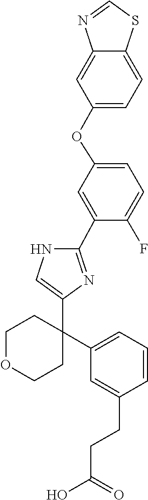 [SMILES: O=C(O)CCc1cccc(C2(c3c[nH]c(-c4cc(Oc5ccc6scnc6c5)ccc4F)n3)CCOCC2)c1] | 166 | 3-(3-(4-(2-(5- (Benzo[d]thiazol-5-yloxy)-2- fluorophenyl)-1H-imidazol-5- yl)tetrahydro-2H-pyran-4- yl)phenyl)propanoic acid | (400 MHz, CD₃OD) δ 9.29 (s, 1H), 8.08 (d, J = 8.8 Hz, 1H), 7.64 (d, J = 2.4 Hz, 1H), 7.62 (dd, J = 6.4, 3.2 Hz, 1H), 7.35 (t, J = 9.6 Hz, 1H), 7.30-7.18 (m, 6H), 7.09 (d, J = 7.2 Hz, 1H), 3.84-3.78 (m, 2H), 3.75-3.69 (m, 2H), 2.90 (t, J = 7.6 Hz, 2H), 2.57 (t, J = 7.6 Hz, 2H), 2.51-2.46 (m, 2H), 2.41-2.34 (m, 2H) ppm. | | 544 [M + H]⁺ |
| 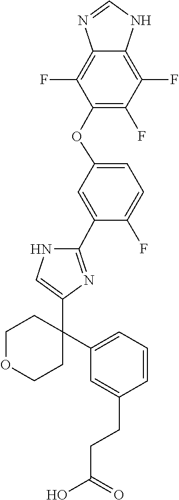 [SMILES: O=C(O)CCc1cccc(C2(c3c[nH]c(-c4cc(Oc5c(F)c(F)c6[nH]cnc6c5F)ccc4F)n3)CCOCC2)c1] | 167 | 3-(3-(4-(2-(2-fluoro-5-((4,6,7- trifluoro-1H-indol-5- yl)oxy)phenyl)-1H-imidazol- 4-yl)tetrahydro-2H-pyran-4- yl)phenyl)propanoic acid | (400 MHz, CD₃OD) δ 7.49 (dd, J = 6.0, 2.8 Hz, 1H), 7.39 (d, J = 3.2 Hz, 1H), 7.24-7.14 (m, 4H), 7.05 (d, J = 7.0 Hz, 1H), 7.00 (s, 1H), 6.99-6.96 (m, 1H), 6.63 (t, J = 3.6 Hz, 1H), 3.82-3.78 (m, 2H), 3.74-3.68 (m, 2H), 2.88 (t, J = 7.6 Hz, 2H), 2.55 (t, J = 7.6 Hz, 2H), 2.51-2.47 (m, 2H), 2.35-2.28 (m, 2H) ppm. | 0.94 | 580 [M + H]⁺ |
| 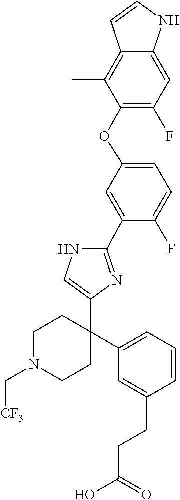 [SMILES: Cc1c(Oc2ccc(F)c(-c3nc(C4(c5cccc(CCC(=O)O)c5)CCN(CC(F)(F)F)CC4)c[nH]3)c2)c(F)cc2[nH]ccc12] | 168 | 3-(3-(4-(2-(2-fluoro-5-((6- fluoro-4-methyl-1H-indol-5- yl)oxy)phenyl)-1H-imidazol- 5-yl)-1-(2,2,2- trifluoroethyl)piperidin-4- yl)phenyl)propanoic acid | (400 MHz, CD₃OD) δ 7.37 (dd, J = 5.6, 2.8 Hz, 1H), 7.27 (d, J = 3.2 Hz, 1H), 7.23 (s, 1H), 7.20-7.09 (m, 4H), 7.04 (d, J = 6.8 Hz, 1H), 6.97 (s, 1H), 6.84-6.78 (m, 1H), 6.52 (d, J = 2.0 Hz, 1H), 3.00 (q, J = 10.0 Hz, 2H), 2.88 (t, J = 8.0 Hz, 2H), 2.84-2.78 (m, 2H), 2.69 (t, J = 10.0 Hz, 2H), 2.56 (t, J = 8.0 Hz, 2H), 2.52-2.48 (m, 2H), 2.42-2.34 (m, 5H) ppm. | 1.05 | 639 [M + H]⁺ |
| 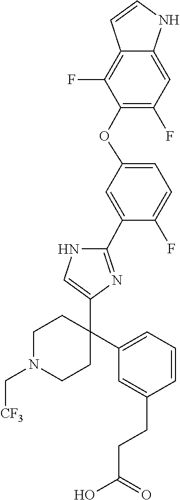 [SMILES: O=C(O)CCc1cccc(C2(c3c[nH]c(-c4cc(Oc5c(F)cc6[nH]ccc6c5F)ccc4F)n3)CCN(CC(F)(F)F)CC2)c1] | 169 | 3-(3-(4-(2-(5-((4,6-difluoro- 1H-indol-5-yl)oxy)-2- fluorophenyl)-1H-imidazol-5- yl)-1-(2,2,2- trifluoroethyl)piperidin-4- yl)phenyl)propanoic acid | (400 MHz, CD₃OD) δ 8 7.46 (dd, J = 6.0, 2.8 Hz, 1H), 7.31 (d, J = 3.2 Hz, 1H), 7.23-7.13 (m, 5H), 7.04 (d, J = 6.8 Hz, 1H), 6.97 (s, 1H), 6.93 (dt, J = 8.8, 3.6 Hz 1H), 6.56 (d, J = 2.8 Hz, 1H), 2.99 (q, J = 10.0 Hz, 2H), 2.88 (t, J = 7.6 Hz, 2H), 2.83-2.79 (m, 2H), 2.69 (t, J = 10.8 Hz, 2H), 2.56 (t, J = 7.6 Hz, 2H), 2.51-2.48 (m, 2H), 2.41-2.34 (m, 2H) ppm. | 1.04 | 643 [M + H]⁺ |
| 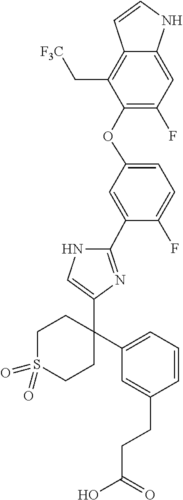 [SMILES: O=C(O)CCc1cccc(C2(c3c[nH]c(-c4cc(Oc5c(F)cc6[nH]ccc6c5CC(F)(F)F)ccc4F)n3)CCS(=O)(=O)CC2)c1] | 170 | 3-(3-(4-(2-(2-fluoro-5-((6- fluoro-4-(2,2,2- trifluoroethyl)-1H-indol-5- yl)oxy)phenyl)-1H-imidazol- 5-yl)-1,1-dioxidotetrahydro- 2H-thiopyran-4- yl)phenyl)propanoic acid | (400 MHz, CD₃OD) δ 7.51 (dd, J = 5.6, 2.8 Hz, 1H), 7.37 (d, J = 3.2 Hz, 1H), 7.33-7.25 (m, 3H), 7.21-7.12 (m, 4H), 6.87 (dt, J = 9.6, 3.2 Hz, 1H), 6.59 (d, J = 3.2 Hz, 1H), 3.76 (q, J = 10.8 Hz, 2H), 3.26-3.19 (m, 2H), 3.07-3.03 (m, 2H), 2.93-2.80 (m, 6H), 2.58 (t, J = 7.6 Hz, 2H) ppm. | 1.07 | 674 [M + H]⁺ |

### Example 171

### 3-(3-(2-(2-(5-((4,6-Difluoro-1H-indol-5-yl)oxy)-2-fluorophenyl)-1H-imidazol-5-yl)-1,3-dimethoxypropan-2-yl)phenyl)propanoic acid

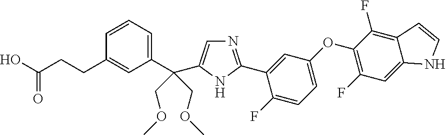 [SMILES: COCC(COC)(c1cccc(CCC(=O)O)c1)c1cnc(-c2cc(Oc3c(F)cc4[nH]ccc4c3F)ccc2F)[nH]1]

### A. 2-(3-Bromophenyl)-3-methoxy-2-(methoxymethyl)propanenitrile

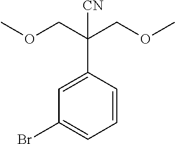

To a stirred and cooled (0° C.) solution of 2-(3-bromophenyl)acetonitrile (10.06 g, 51.3 mmol) in DMF (100 mL) was added, portionwise over 10 minutes, a 60% dispersion of sodium hydride in mineral oil (6.15 g, 154 mmol). Following the addition, the frothy mixture was stirred for another 20 minutes at 0° C. before adding methoxymethyl bromide (10.4 mL, 127 mmol). The reaction was warmed to room temperature and stirred overnight. After this time, the reaction was quenched with the dropwise addition of water (˜5 mL) and then partitioned between ethyl acetate (300 mL) and water (100 mL). The organic layer was washed with additional water (2×100 mL) and brine (1×100 mL), dried over sodium sulfate and concentrated. The residue was purified by flash chromatography over silica (10-20% ethyl acetate in petroleum ether) to afford the title compound as a colorless oil (3.37 g, 23%). MS: 284 m/z [M+H]⁺.

### B. 2-(3-Bromophenyl)-3-methoxy-2-(methoxymethyl)propanoic acid

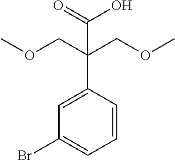

To a stirred solution of step A product (2.81 g, 9.90 mmol) in 6:1 ethanol/water (35 mL) was added potassium hydroxide (16.6 g, 296 mmol). The reaction was heated overnight at 100° C. and then cooled to room temperature and concentrated. The residue was dissolved in water (50 mL) and this stirred and cooled (0° C.) solution was slowly treated with 3.0 N hydrochloric acid until an acidic pH was achieved (˜100 mL). The mixture was then extracted with ethyl acetate (4×50 mL) and the combined extracts were washed with water (1×100 mL) and brine (1×100 mL) and dried over sodium sulfate. The solution was concentrated to afford a residue which was purified by flash chromatography over silica (15-25% ethyl acetate in petroleum ether). The title compound was obtained as a white solid as a white solid (2.22 g, 74%). MS: 303 m/z [M+H]⁺.

### C. 2-(3-Bromophenyl)-N,3-dimethoxy-2-(methoxymethyl)-N-methylpropanamide

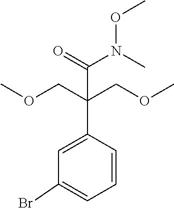

To a stirred and cooled (0° C.) solution of step B product (1.20 g, 3.97 mmol) in dichloromethane (15 mL) was added oxalyl dichloride (1.02 mL, 11.9 mmol) followed by two drops of DMF. The mixture was allowed to warm to room temperature, stirred for two hours and then concentrated. A solution of the crude acid chloride in dichloromethane (5 mL) was added, dropwise over 3-4 minutes, to a stirred and cooled (0° C.) solution of N,O-dimethylhydroxylamine hydrochloride (0.578 g, 5.93 mmol) and triethylamine (1.7 mL, 12 mmol) in dichloromethane (15 mL). The reaction was warmed to room temperature, stirred for an additional two hours and then partitioned between dichloromethane (50 mL) and water (50 mL). The organic layer was washed with additional water (1×50 mL) and brine (1×50 mL), dried over sodium sulfate and concentrated. The residue was purified by flash chromatography over silica (15-25% ethyl acetate in petroleum ether) to afford the title compound as a white solid (1.20 g, 87%). MS: 346, 348 m/z [M+H]⁺.

### D. 3-(3-Bromophenyl)-4-methoxy-3-(methoxymethyl)butan-2-one

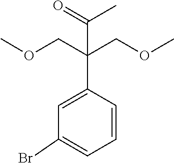

To a stirred and cooled (0° C.) solution of step C product (1.20 g, 3.47 mmol) in THF (20 mL) was added, dropwise over five minutes, a 3.0 M solution of methylmagnesium bromide in THF (11.0 mL, 33.0 mmol). The reaction was warmed to room temperature, stirred for three hours, and then quenched with the addition of saturated aqueous ammonium chloride solution (dropwise initially; 50 mL total). The resulting suspension was extracted with ethyl acetate (3×50 mL) and the combined extracts were washed with brine (1×50 mL), dried over sodium sulfate and concentrated. The obtained residue was subjected to flash chromatography over silica (10-25% ethyl acetate in petroleum ether) to afford the title compound as a white solid (0.899 g, 86%). MS: 301, 303 m/z [M+H]⁺.

### E. 1-Bromo-3-(3-bromophenyl)-4-methoxy-3-(methoxymethyl)butan-2-one

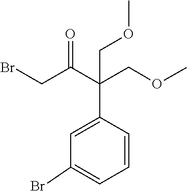 [SMILES: COCC(COC)(C(=O)CBr)c1cccc(Br)c1]

To a stirred and cooled (0° C.) solution of step D product (0.350 g, 1.16 mmol) in THF (5 mL) was added pyridinium tribromide (0.740 g, 2.31 mmol). The reaction was heated overnight at 75° C. and then cooled to room temperature and concentrated. A mixture of the crude title compound and pyridine hydromide, which was used without purification, was obtained as a yellow solid (0.700 g).

### F. 5-(3-(5-(2-(3-Bromophenyl)-1,3-dimethoxypropan-2-yl)-1H-imidazol-2-yl)-4-fluorophenoxy)-4,6-difluoro-1H-indole

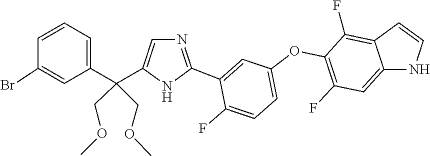 [SMILES: COCC(COC)(c1cccc(Br)c1)c1cnc(-c2cc(Oc3c(F)cc4[nH]ccc4c3F)ccc2F)[nH]1]

Exchanging methyl 3-(3-(4-(2-bromoacetyl)-2,6-dimethyltetrahydro-2H-pyran-4-yl)phenyl)propanoate for impure step E product (1.16 mmol, assuming complete conversion in the previous step), a procedure analogous to that described in step J of the Example 142 synthesis was used to prepare the title compound as a yellow solid (0.320 g, 47% for the two steps). MS: 586 m/z [M+H]⁺.

### G. Example 171, 3-(3-(2-(2-(5-((4,6-Difluoro-1H-indol-5-yl)oxy)-2-fluorophenyl)-1H-imidazol-5-yl)-1,3-dimethoxypropan-2-yl)phenyl)propanoic acid

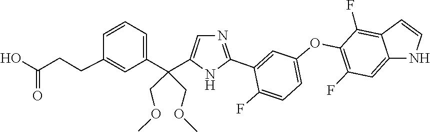 [SMILES: COCC(COC)(c1cccc(CCC(=O)O)c1)c1cnc(-c2cc(Oc3c(F)cc4[nH]ccc4c3F)ccc2F)[nH]1]

Exchanging (5-(3-(4-(3-iodobenzyl)thiazol-2-yl)phenoxy)-1H-indol-4-yl)methanol for step F product, procedures analogous to those described in steps D-F of the Example 99 synthesis were used to prepare the title compound as a white solid. ¹H NMR (400 MHz, CD₃OD) δ 7.49 (s, 1H), 7.31 (d, J=2.8 Hz, 1H), 7.25-7.04 (m, 6H), 6.96-6.83 (m, 2H), 6.56 (s, 1H), 4.02 (d, J=9.2 Hz, 2H), 3.91 (d, J=9.2 Hz, 2H), 3.35 (s, 6H), 2.87 (t, J=7.6 Hz, 2H), 2.54 (t, J=7.6 Hz, 2H) ppm. MS: 580 m/z [M+H]⁺.

### Examples 172 and 173

### 2-Fluoro-3-(3-(hydroxy(2-(3-((4-methyl-1H-indol-5-yl)oxy)phenyl)-1H-imidazol-5-yl)methyl)phenyl)propanoic acid

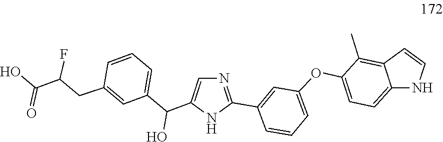 [SMILES: Cc1c(Oc2cccc(-c3ncc(C(O)c4cccc(CC(F)C(=O)O)c4)[nH]3)c2)ccc2[nH]ccc12]

### Methyl 2-fluoro-3-(3-(hydroxy(2-(3-((4-methyl-1H-indol-5-yl)oxy)phenyl)-1H-imidazol-5-yl)methyl)phenyl)propanoate

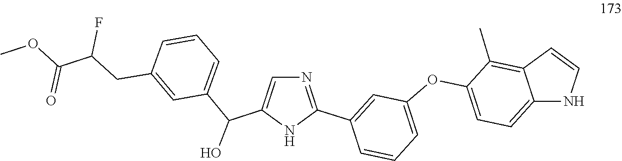 [SMILES: COC(=O)C(F)Cc1cccc(C(O)c2cnc(-c3cccc(Oc4ccc5[nH]ccc5c4C)c3)[nH]2)c1]

### A. 2-(3-Bromophenyl)acetaldehyde

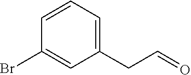

To a stirred solution of 2-(3-bromophenyl)ethan-1-ol (15 g, 75 mmol) in dichloromethane (450 mL) was added Dess-Martin periodinane (38 g, 90 mmol) portion wise at room temperature. The reaction mixture was stirred for 30 minutes, quenched with of a solution of sodium bicarbonate (20.7 g, 246 mmol) and sodium thiosulfate (71.1 g, 450 mmol) in water (450 mL), stirred for 30 minutes and extracted with dichloromethane. The combined organic extracts were washed with brine, dried over sodium sulfate and concentrated to give the crude title compound (15 g) which was used for the next step without further purification. ¹H NMR (400 MHz, CDCl₃) δ 9.74 (t, J=2.4 Hz, 1H), 7.44 (d, J=8.4 Hz, 1H), 7.38 (s, 1H), 7.24 (t, J=8 Hz, 1H), 7.15 (d, J=7.6 Hz, 1H), 3.68 (d, J=2.4 Hz, 1H) ppm.

### B. 3-(3-Bromophenyl)-2-((trimethylsilyl)oxy)propanenitrile

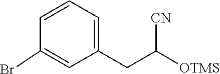

To a stirred mixture of 2-(3-bromophenyl)acetaldehyde (15 g, 75 mmol) and zinc iodide (958 mg, 3 mmol) in dichloromethane (150 mL) was added trimethylsilylcyanide (14.85 g, 150 mmol) dropwise at 0° C. under nitrogen atmosphere. The reaction mixture was stirred at room temperature for one hour, diluted with dichloromethane (200 mL), washed with brine, dried over sodium sulfate and concentrated to give the title compound (22.5 g), which was used in the next step without further purification.

### C. Methyl 3-(3-bromophenyl)-2-hydroxypropanoate

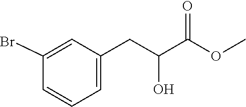

A solution of 3-(3-bromophenyl)-2-((trimethylsilyl)oxy)propanenitrile (22.5 g, 75 mmol) in hydrochloric acid (3M in methanol, 300 mL) was stirred at 85° C. for two days and then concentrated to remove methanol. The resulting residue was diluted with water (100 mL), neutralized with saturated sodium bicarbonate solution to pH˜8 and extracted with dichloromethane (100 mL×4). The combined organic phase was dried over sodium sulfate and concentrated. The resulting residue was purified by silica gel chromatography to give the title compound (14.5 g, 75% from 3 steps). MS: 259, 261 m/z [M+H]⁺.

### D. Methyl 3-(3-bromophenyl)-2-fluoropropanoate

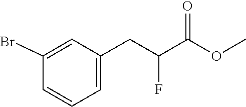

To a stirred solution of methyl 3-(3-bromophenyl)-2-hydroxypropanoate (8.0 g, 31 mmol) in dichloromethane (200 mL) was added a solution of DAST (7.5 g, 46.5 mmol) in dichloromethane (200 mL) dropwise at −10° C. under nitrogen atmosphere. The resulting mixture was stirred at room temperature for three hours, poured into an ice-water mixture, and extracted with ethyl acetate (100 mL×3). The combined organic extracts were washed with brine, dried over sodium sulfate and concentrated. The resulting residue was purified by silica gel chromatography to give the title compound as a yellow oil (5.3 g, 66%).

### E. Methyl 3-(3-(3-acetoxyprop-1-en-1-yl)phenyl)-2-fluoropropanoate

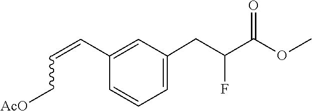 [SMILES: COC(=O)C(F)Cc1cccc(C=CCOC(C)=O)c1]

A mixture of methyl 3-(3-bromophenyl)-2-fluoropropanoate (5 g, 19.2 mmol), allyl acetate (5.8 g, 57.6 mmol), palladium(II) acetate (862 mg, 3.84 mmol) and silver carbonate (6.32 g, 23 mmol) in toluene (250 mL) was stirred at 100° C. for 0.5 hours, filtered and concentrated. The resulting residue was purified by flash chromatography over silica (7-30% ethyl acetate in petroleum ether) to give methyl 3-(3-(3-acetoxyprop-1-en-1-yl)phenyl)-2-fluoropropanoate (453 mg, 8.3%) together with 4.5 g of methyl 3-(3-bromophenyl)-2-fluoropropanoate. MS: 281 m/z [M+H]⁺.

### F. Methyl 2-fluoro-3-(3-(3-hydroxyprop-1-en-1-yl)phenyl)propanoate

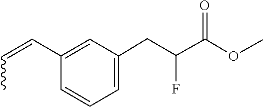

To a stirred solution of methyl (Z)-3-(3-(3-acetoxyprop-1-en-1-yl)phenyl)-2-fluoropropanoate (850 mg, 3 mmol) in methanol (70 mL) was added potassium carbonate (419 mg, 3 mmol). The reaction mixture was stirred at room temperature for 0.5 hour, concentrated, diluted with water (20 mL) and extracted with dichloromethane. The combined organic extracts were washed with brine, dried over sodium sulfate and concentrated to give the title compound as a yellow oil (550 mg, 77%), which was used for next step without further purification. MS: 239 m/z [M+H]⁺.

### G. Methyl 2-fluoro-3-(3-(3-(hydroxymethyl)oxiran-2-yl)phenyl)propanoate

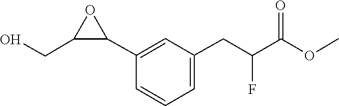 [SMILES: COC(=O)C(F)Cc1cccc(C2OC2CO)c1]

A mixture of methyl 2-fluoro-3-(3-(3-hydroxyprop-1-en-1-yl)phenyl)propanoate (550 mg, 2.3 mmol), meta-chloroperoxybenzoic acid (800 mg, 2.3 mmol) and sodium bicarbonate (231 mg, 2.75 mmol) in dichloromethane (43 mL) was stirred at room temperature for 24 hours. The reaction was quenched with aqueous sodium thiosulfate, stirred for 30 minutes and extracted with a 1:4 mixture of ethyl acetate and dichloromethane (30 mL×2). The combined organic extracts were washed with brine, dried over sodium sulfate and concentrated. The resulting residue was purified by flash chromatography over silica (0-11% ethyl acetate in dichloromethane) to give the title compound as yellow syrup (337 mg, 58%). MS: 255 m/z [M+H]⁺.

### H. Methyl 2-fluoro-3-(3-(3-formyloxiran-2-yl)phenyl)propanoate

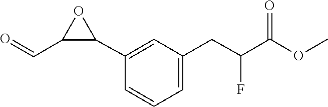 [SMILES: COC(=O)C(F)Cc1cccc(C2OC2C=O)c1]

To a stirred solution of methyl 2-fluoro-3-(3-(3-(hydroxymethyl)oxiran-2-yl)phenyl)propanoate (230 mg, 0.9 mmol) in dichloromethane (14 mL) was added BAIB (322 mg, 1.1 mmol) and TEMPO (14 mg, 0.09 mmol). The reaction mixture was stirred at room temperature for two hours and concentrated. The resulting residue was purified by silica gel chromatography (dichloromethane/ethyl acetate, v/v, 8/1) to give the title compound as a light yellow oil (133 mg, 59%).

### I. 3-((4-Methyl-1H-indol-5-yl)oxy)benzimidamide

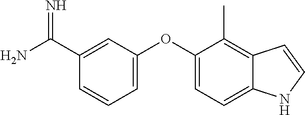 [SMILES: Cc1c(Oc2cccc(C(=N)N)c2)ccc2[nH]ccc12]

To a stirred solution of 3-((4-methyl-1H-indol-5-yl)oxy)benzonitrile (150 mg, 0.6 mmol) in THF (7 mL) was added lithium bis(trimethylsilyl)amide (1M in THF, 2.4 mL, 2.4 mmol) dropwise under nitrogen. The reaction mixture was stirred at room temperature overnight, quenched with water (30 mL) and extracted with a 1:1 mixture of THF and ethyl acetate (25 mL×3). The combined organic extracts were washed with brine, dried over sodium sulfate and concentrated to give the title compound as a brown solid (158 mg), which was used for next step without further purification. MS: 266 m/z [M+H]⁺.

### J. Example 173. Methyl 2-fluoro-3-(3-(hydroxy(2-(3-((4-methyl-1H-indol-5-yl)oxy)phenyl)-1H-imidazol-5-yl)methyl)phenyl)propanoate

A stirred solution of methyl 2-fluoro-3-(3-(3-formyloxiran-2-yl)phenyl)propanoate (133 mg, 0.53 mmol) and 3-((4-methyl-1H-indol-5-yl)oxy)benzimidamide (155 mg, 0.58 mmol) in DMF (8 mL) was stirred at 75° C. overnight. The reaction mixture was cooled to room temperature, diluted with water (20 mL) and extracted with ethyl acetate (25 mL×4). The combined organic extracts were washed with brine (25 mL×2), dried over sodium sulfate and concentrated. The resulting residue was purified by flash chromatography over silica (0-3% methanol in dichloromethane) to afford the title compound as a brown solid (75 mg, 28%). MS: 500 m/z [M+H]⁺. ¹H NMR (400 MHz, CD₃OD) δ 7.46 (d, J=7.6 Hz, 1H), 7.37-7.26 (m, 7H), 7.18 (d, J=5.6 Hz, 1H), 6.84-6.80 (m, 2H), 6.73 (s, 1H), 6.53 (d, J=3.2 Hz, 1H), 5.79 (s, 1H), 5.29-5.14 (m, 1H), 3.68 (d, J=3.6 Hz, 3H), 3.28-3.09 (m, 2H), 2.34 (s, 3H) ppm.

### K. Example 172. 2-Fluoro-3-(3-(hydroxy(2-(3-((4-methyl-1H-indol-5-yl)oxy)phenyl)-1H-imidazol-5-yl)methyl)phenyl)propanoic acid

To a stirred solution of methyl 2-fluoro-3-(3-(hydroxy(2-(3-((4-methyl-1H-indol-5-yl)oxy)phenyl)-1H-imidazol-5-yl)methyl)phenyl)propanoate (50 mg, 0.1 mmol) in a 2:1 mixture of THF and water (2.4 mL) was added lithium hydroxide (17 mg, 0.7 mmol). The reaction mixture was stirred at room temperature overnight, concentrated, diluted with water (1 mL) and acidified with 1N hydrochloric acid to pH˜2. The precipitate was collected by filtration and purified by preparative HPLC to give the title compound as a white solid (28 mg, 56%). MS: 486 m/z [M+H]⁺. ¹H NMR (400 MHz, CD₃OD) δ 7.47 (d, J=7.6 Hz, 1H), 7.42-7.40 (m, 2H), 7.35 (s, 1H), 7.33-7.26 (m, 5H), 6.95-6.93 (s, 2H), 6.81 (d, J=8.4 Hz, 1H), 6.51 (d, J=4.8 Hz, 1H), 5.80 (d, J=2.4 Hz, 1H), 4.99-4.85 (m, 1H), 3.24-3.04 (m, 2H), 2.34 (s, 3H) ppm.

The following examples were prepared utilizing similar procedure as described in Examples 172 and 173.

| | Ex. | | | LCMS | MS m/z |
|---|---|---|---|---|---|
| Structure | No. | Name | ¹H NMR | Rₜ (min) | [M + H]⁺ |
| 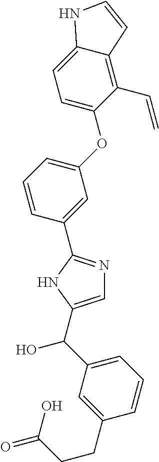 [SMILES: C=Cc1c(Oc2cccc(-c3ncc(C(O)c4cccc(CCC(=O)O)c4)[nH]3)c2)ccc2[nH]ccc12] | 174 | 3-(3-(hydroxy(2-(3- ((4-vinyl-1H-indol-5- yl)oxy)phenyl)-1H- imidazol-5- yl)methyl)phenyl) propanoic acid | (400 MHz, CD₃OD) δ 7.47 (d, J = 7.6 Hz, 1H), 7.41 (s, 1H), 7.38-7.31 (m, 4H), 7.28 (d, J = 4.4 Hz, 2H), 7.17-7.19 (m, 1H), 7.09 (dd, J = 11.6, 18.0 Hz, 1H), 6.84-6.82 (d, J = 8.8 Hz, 2H), 6.79 (d, J = 2.8 Hz, 1H), 6.76 (s, 1H), 5.95 (t, J = 18.0 Hz, 1H), 5.78 (s, 1H), 5.42 (d, J = 12.4 Hz, 1H), 2.93 (t, J = 7.6 Hz, 2H), 2.61 (t, J = 3.0 Hz, 2H) ppm. | 0.83 | 480 |
| 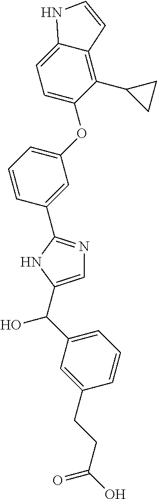 [SMILES: O=C(O)CCc1cccc(C(O)c2cnc(-c3cccc(Oc4ccc5[nH]ccc5c4C4CC4)c3)[nH]2)c1] | 175 | 3-(3-((2-(3-((4- cyclopropyl-1H- indol-5- yl)oxy)phenyl)-1H- imidazol-5- yl)(hydroxy)methyl) phenyl)propanoic acid | (400 MHz, CD₃OD) δ 7.45 (d, J = 7.6 Hz, 1H), 7.40 (s, 1H), 7.35-7.25 (m, 6H), 7.17 (d, J = 4.0 Hz, 1H), 6.84-6.76 (m, 3H), 6.60 (d, J = 2.4 Hz, 1H), 5.78 (s, 1H), 2.93 (t, J = 7.6 Hz, 2H), 2.58 (t, J = 7.2 Hz, 2H), 1.97-1.95 (m, 1H), 0.97-0.95 (m, 2H), 0.85-0.82 (m, 2H) ppm. | 0.86 | 494 |
| 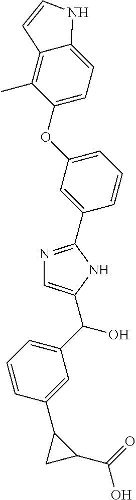 [SMILES: Cc1c(Oc2cccc(-c3ncc(C(O)c4cccc(C5CC5C(=O)O)c4)[nH]3)c2)ccc2[nH]ccc12] | 176 | 2-(3-(hydroxy(2-(3- ((4-methyl-1H-indol- 5-yl)oxy)phenyl)-1H- imidazol-5- yl)methyl)phenyl) cyclopropane-1- carboxylic acid | (400 MHz, CD₃OD) δ 7.47 (d, J = 7.2 Hz, 1H), 7.40-7.37 (m, 2H), 7.29- 7.27 (m, 5H), 7.09 (d, J = 3.6 Hz, 1H), 6.90-6.81 (m, 3H), 6.52 (d, J = 2.0 Hz, 1H), 5.80 (s, 1H), 2.52-2.47 (m, 1H), 2.35 (s, 3H), 1.88-1.83 (m, 1H), 1.57-1.52 (m, 1H), 1.40-1.35 (m, 1H) ppm | 0.85 | 480 |
| 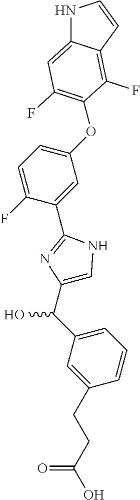 [SMILES: O=C(O)CCc1cccc(C(O)c2c[nH]c(-c3cc(Oc4c(F)cc5[nH]ccc5c4F)ccc3F)n2)c1] | 177 | 3-(3-((2-(5-((4,6- difluoro-1H-indol-5- yl)oxy)-2- fluorophenyl)-1H- imidazol-4- yl)(hydroxy)methyl) phenyl)propanoic acid | (400 MHz, CD₃OD) δ 7.51 (dd, J = 3.2, 5.6 Hz, 1H), 7.34 (s, 1H), 7.31 (d, J = 2.8 Hz, 1H), 7.13-7.27 (m, 5H), 7.00 (dt, J = 8.8, 3.6 Hz, 1H), 6.80 (s, 1H), 6.56 (d, J = 2.8 Hz, 1H), 5.77 (s, 1H), 2.92 (t, J = 7.6 Hz, 2H), 2.58 (t, J = 7.2 Hz, 2H) ppm | 0.84 | 508 |
| 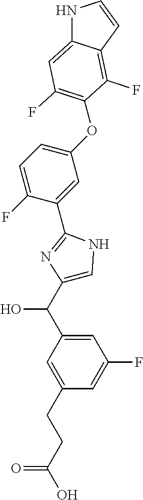 [SMILES: O=C(O)CCc1cc(F)cc(C(O)c2c[nH]c(-c3cc(Oc4c(F)cc5[nH]ccc5c4F)ccc3F)n2)c1] | 178 | 3-(3-((2-(5-((4,6- difluoro-1H-indol-5- yl)oxy)-2- fluorophenyl)-1H- imidazol-4- yl)(hydroxy)methyl)- 5-fluorophenyl) propanoic acid | (400 MHz, CD₃OD) δ 7.52 (m, 1H), 7.31 (d, J = 3.6 Hz, 1H), 7.23-7.13 (m, 3H), 7.04-6.98 (m, 2H), 6.92 (d, J = 10 Hz, 1H), 6.84 (s, 1H), 6.56 (d, J = 3.2 Hz, 1H), 5.76 (s, 1H), 2.93 (t, J = 7.6 Hz, 2H), 2.61 (t, J = 7.6 Hz, 2H) ppm | 0.88 | 526 |
| 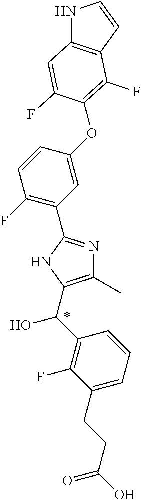 [SMILES: Cc1nc(-c2cc(Oc3c(F)cc4[nH]ccc4c3F)ccc2F)[nH]c1C(O)c1cccc(CCC(=O)O)c1F] | 179a | Enantiomer 1 of 3-(3- ((2-(5-((4,6-difluoro- 1H-indol-5-yl)oxy)- 2-fluorophenyl)-4- methyl-1H-imidazol- 5- yl)(hydroxy)methyl)- 2- fluorophenyl) propanoic acid | (400 MHz, CD₃OD) δ 7.53 (t, J = 7.5 Hz, 1H), 7.47 (dd, J = 5.9, 3.2 Hz, 1H), 7.31 (d, J = 3.2 Hz, 1H), 7.22- 7.08 (m, 4H), 6.97 (dd, J = 8.2, 4.3 Hz, 1H), 6.56 (d, J = 3.1 Hz, 1H), 6.14 (s, 1H), 2.94 (t, J = 7.6 Hz, 2H), 2.57 (t, J = 7.7 Hz, 2H), 2.12 (s, 3H) ppm. | 0.84 | 540 |
| 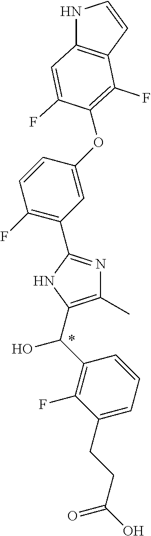 [SMILES: Cc1nc(-c2cc(Oc3c(F)cc4[nH]ccc4c3F)ccc2F)[nH]c1C(O)c1cccc(CCC(=O)O)c1F] | 180^{a} | Enantiomer 2 of 3-(3- ((2-(5-((4,6-difluoro- 1H-indol-5-yl)oxy)- 2-fluorophenyl)-4- methyl-1H-imidazol- 5-yl)(hydroxy)methyl)- 2-fluorophenyl) propanoic acid | (400 MHz, CD₃OD) δ 7.53 (t, J = 7.5 Hz, 1H), 7.47 (dd, J = 5.9, 3.2 Hz, 1H), 7.31 (d, J = 3.2 Hz, 1H), 7.22- 7.08 (m, 4H), 6.97 (dd, J = 8.2, 4.3 Hz, 1H), 6.56 (d, J = 3.1 Hz, 1H), 6.14 (s, 1H), 2.94 (t, J = 7.6 Hz, 2H), 2.57 (t, J = 7.7 Hz, 2H), 2.12 (s, 3H) ppm. | 0.84 | 540 |

^{a}: SFC separation conditions: Instrument: SFC-80 (Thar, Waters); Column: (R,R)-WHELK-01 (20 × 250 mm, 10 micron); Column Temperature: 35° C. Mobile phase: CO₂/ethanol(0.2% ammonia saturated methanol) = 50/50; Flow rate: 80 g/min. Back pressure: 100 bar.

### Example 181

### 3-(3-((S)-(2-(3-((4,6-Difluoro-1H-indol-5-yl)oxy)phenyl)-1H-imidazol-5-yl)(hydroxy)methyl)phenyl)-2-methylpropanoic acid

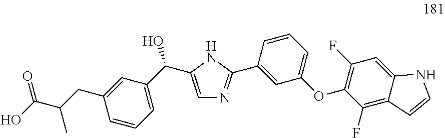 [SMILES: CC(Cc1cccc([C@H](O)c2cnc(-c3cccc(Oc4c(F)cc5[nH]ccc5c4F)c3)[nH]2)c1)C(=O)O]

### A. Ethyl (E)-3-(3-iodophenyl)-2-methylacrylate

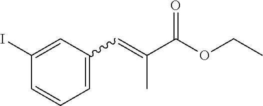

To a stirred solution of ethyl 2-(diethoxyphosphoryl)propanoate (492 mg, 2.07 mmol) in THF (17 mL) was added lithium bis(trimethylsilyl)amide (1 M in THF, 2.07 mL, 2.07 mmol) at 0° C. The reaction mixture was stirred for 30 minutes, a solution of 3-iodobenzaldehyde (400 mg, 1.72 mmol) in dry THF (3 mL) was added dropwise and stirred for another two hours. The reaction mixture was quenched with saturated ammonium chloride solution (20 mL) and extracted with ethyl acetate (30 mL×3). The combined organic extracts were washed with brine (20 mL), dried over sodium sulfate, filtered and concentrated to afford a residue, which was purified by silica gel chromatography (petroleum ether/ethyl acetate, v/v, 100/1) to afford the title compound as a light yellow oil (450 mg, 83%). ¹H NMR (400 MHz, DMSO-d₆) δ 7.81 (s, 1H), 7.74 (d, J=8 Hz, 1H), 7.54 (s, 1H), 7.5 (d, J=7.6 Hz, 1H), 7.24 (t, J=7.6 Hz, 1H), 4.21 (q, J=7.2 Hz, 2H), 2.02 (s, 3H), 1.29 (t, J=7.2 Hz, 3H) ppm.

### B. Ethyl 3-(3-((E)-3-acetoxyprop-1-en-1-yl)phenyl)-2-methylacrylate

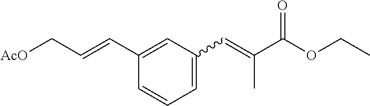 [SMILES: CCOC(=O)C(C)=Cc1cccc(/C=C/COC(C)=O)c1]

A mixture of ethyl (E)-3-(3-iodophenyl)-2-methylacrylate (4.5 g, 14.24 mmol), allyl acetate (4.27 g, 42.72 mmol), silver carbonate (1.95 g, 8.54 mmol) and palladium(II) acetate (786 mg, 2.85 mmol) in toluene (25 mL) was stirred at 80° C. for three hours, cooled to room temperature and filtered through Celite. The filtrate was concentrated and the resulting residue was purified by flash chromatography over silica (petroleum ether/ethyl acetate, v/v, 15/1) to afford the title compound as a light yellow oil (3.3 g, 80%). MS: 289 m/z [M+H]⁺.

### C. Ethyl 3-(3-((E)-3-hydroxyprop-1-en-1-yl)phenyl)-2-methylacrylate

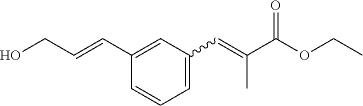 [SMILES: CCOC(=O)C(C)=Cc1cccc(/C=C/CO)c1]

A mixture of ethyl 3-(3-((E)-3-acetoxyprop-1-en-1-yl)phenyl)-2-methylacrylate (110 mg, 0.24 mmol) and potassium carbonate (295 mg, 2.15 mmol) in ethanol (5 mL) was stirred at room temperature for two hours, diluted with water (20 mL) and extracted with dichloromethane (20 mL×3). The combined organic extracts were dried over sodium sulfate, filtered and concentrated. The resulting residue was purified by flash chromatography over silica (petroleum ether/ethyl acetate, v/v, 10/1-1/1) to afford the titile compound as a colorless oil (230 mg, 80%). MS: 247 m/z [M+H]⁺.

### D. Ethyl 3-(3-((2S,3S)-3-(hydroxymethyl)oxiran-2-yl)phenyl)-2-methylacrylate

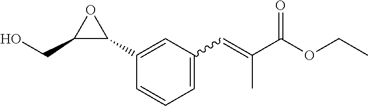 [SMILES: CCOC(=O)C(C)=Cc1cccc([C@H]2O[C@@H]2CO)c1]

To a stirred mixture of (R,R)-(+)-diisopropyl tartrate (71 mg, 0.3 mmol), titanium (IV) isopropoxide (58 mg, 0.2 mmol) and 4 Å molecular sieves (800 mg) in dry dichloromethane (30 mL), was added a solution of tert-butyl hydroperoxide (6M in toluene, 1.4 mL, 8.14 mmol) dropwise at −20° C. over fifteen minutes and then stirred at this temperature for one hour. A solution of ethyl 3-(3-((E)-3-hydroxyprop-1-en-1-yl)phenyl)-2-methylacrylate (1 g, 4.07 mmol) in dry dichloromethane (10 mL) was added dropwise over thirty minutes, stirred for three hours at −20° C., quenched with 10% aqueous sodium hydroxide solution saturated with sodium chloride (1 mL) and diluted with ethyl acetate (50 mL). The cold bath was then removed, the reaction mixture was allowed to warm to 10° C. and stirring was maintained at 10° C. while magnesium sulfate (3 g) and Celite (300 mg) were added. After another fifteen minutes of stirring, the mixture was allowed to settle, the clean solution was filtered through a pad of Celite and washed with ethyl acetate. The filtrate was concentrated and the resulting residue was purified by flash chromatography over silica (petroleum ether/ethyl acetate, v/v, 5/1) to afford the title compound as a colorless oil (955 mg, 90%). MS: 263 m/z [M+H]⁺.

### E. Ethyl 3-(3-((2S,3R)-3-formyloxiran-2-yl)phenyl)-2-methylacrylate

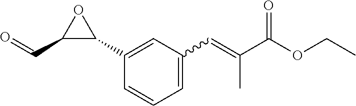 [SMILES: CCOC(=O)C(C)=Cc1cccc([C@H]2O[C@@H]2C=O)c1]

A stirred solution of ethyl 3-(3-((2S,3S)-3-(hydroxymethyl)oxiran-2-yl)phenyl)-2-methylacrylate (950 mg, 3.63 mmol), BAIB (1.28 g, 3.99 mmol) and TEMPO (57 mg, 0.36 mmol) in dichloromethane (10 mL) was stirred at room temperature for two hours. The reaction mixture was concentrated and the residue was purified by flash chromatography over silica (petroleum ether/ethyl acetate, v/v, 8/1) to afford the title compound as a colorless oil (590 mg, 62%). ¹H NMR (400 MHz, CDCl₃) δ 9.23 (d, J=6 Hz, 1H), 7.67 (s, 1H), 7.44 (m, 2H), 7.32 (s, 1H), 7.28 (m, 1H), 4.33-4.27 (q, J=13 Hz, 2H), 4.22 (m, 1H), 3.47 (d, J=6.4 Hz, 1H), 2.11 (s, 3H), 1.41-1.27 (t, J=13 Hz, 3H) ppm.

### F. Ethyl (S)-3-(3-((2-(3-((4,6-difluoro-1H-indol-5-yl)oxy)phenyl)-1H-imidazol-5-yl)(hydroxy)methyl)phenyl)-2-methylacrylate

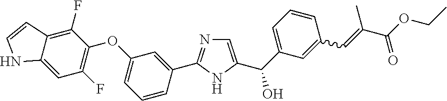 [SMILES: CCOC(=O)C(C)=Cc1cccc([C@H](O)c2cnc(-c3cccc(Oc4c(F)cc5[nH]ccc5c4F)c3)[nH]2)c1]

A mixture of ethyl 3-(3-((2S,3R)-3-formyloxiran-2-yl)phenyl)-2-methylacrylate (650 mg, 2.26 mmol) and 3-((4,6-difluoro-1H-indol-5-yl)oxy)benzimidamide (Intermediate 24-2, 589 mg, 2.26 mmol) in DMF (10 mL) was stirred at 80° C. for six hours. The reaction mixture was concentrated and the resulting residue was purified by preparative TLC (dichloromethane/methanol, v/v, 20/1) to afford the title compound as a yellow solid (270 mg, 22%). MS: 530 m/z [M+H]⁺.

### G. Ethyl 3-(3-((S)-(2-(3-((4,6-difluoro-1H-indol-5-yl)oxy)phenyl)-1H-imidazol-5-yl)(hydroxy)methyl)phenyl)-2-methylpropanoate

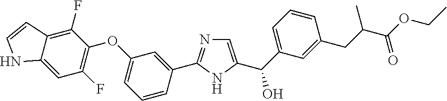 [SMILES: CCOC(=O)C(C)Cc1cccc([C@H](O)c2cnc(-c3cccc(Oc4c(F)cc5[nH]ccc5c4F)c3)[nH]2)c1]

To a stirred solution of ethyl (S)-3-(3-((2-(3-((4,6-difluoro-1H-indol-5-yl)oxy)phenyl)-1H-imidazol-5-yl)(hydroxy)methyl)phenyl)-2-methylacrylate (100 mg, 0.19 mmol) and nickel chloride hexahydrate (115 mg, 0.57 mmol) in ethanol (5 mL) was added sodium borohydride (44 mg, 1.14 mmol) at room temperature. The reaction mixture was stirred for three hours, quenched with water (0.2 mL) and filtered. The filtrate was concentrated and the resulting residue was purified by preparative HPLC to afford the title compound as a colorless oil (40 mg, 40%). MS: 532 m/z [M+H]⁺. ¹H NMR (400 MHz, CD₃OD) δ 7.55 (d, J=7.6 Hz, 1H), 7.48 (s, 1H), 7.40 (t, J=8 Hz, 1H), 7.31-7.27 (m, 4H), 7.16 (m, 2H), 6.96-6.94 (m, 1H), 6.78 (d, J=5.6 Hz, 1H), 6.56 (d, J=3.2 Hz, 1H), 5.78 (s, 1H), 4.00-3.97 (m, 2H), 3.94 (m, 1H), 2.75 (m, 2H), 1.16-1.08 (m, 6H) ppm.

### H. Example 181. 3-(3-((S)-(2-(3-((4,6-Difluoro-1H-indol-5-yl)oxy)phenyl)-1H-imidazol-5-yl)(hydroxy)methyl)phenyl)-2-methylpropanoic acid

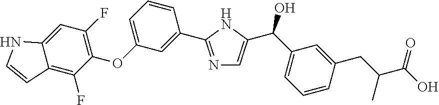 [SMILES: CC(Cc1cccc([C@H](O)c2cnc(-c3cccc(Oc4c(F)cc5[nH]ccc5c4F)c3)[nH]2)c1)C(=O)O]

To a stirred solution of ethyl 3-(3-((S)-(2-(3-((4,6-difluoro-1H-indol-5-yl)oxy)phenyl)-1H-imidazol-5-yl)(hydroxy)methyl)phenyl)-2-methylpropanoate (45 mg, 0.085 mmol) in THF (3 mL) was added a solution of lithium hydroxide (20 mg, 0.85 mmol) in water (2 mL). The reaction mixture was stirred at 50° C. overnight, quenched with 1 M hydrochloric acid (0.9 mL, 0.9 mmol) and extracted with ethyl acetate (20 mL×3). The combined organic extracts were washed with brine (20 mL), dried over sodium sulfate, filtered and concentrated. The resulting residue was purified by preparative HPLC to afford the title compound as a yellow solid (10 mg, 23%). MS: 504 m/z [M+H]⁺. ¹H NMR (400 MHz, CD₃OD) δ 7.55 (d, J=7.6 Hz, 1H), 7.47 (s, 1H), 7.40-7.36 (t, J=8 Hz, 1H), 7.32-7.26 (m, 4H), 7.15 (d, J=9.6 Hz, 2H), 6.93 (dd, J=2.0, 9.6 Hz, 1H), 6.73 (s, 1H), 6.56 (d, J=2.8 Hz, 1H), 5.77 (s, 1H), 3.10-3.00 (m, 1H), 2.68-2.66 (m, 2H), 1.13 (d, J=4.8 Hz, 3H) ppm.

### Example 182

### 3-(3-((S)-(2-(3-((4,6-Difluoro-1H-indol-5-yl)oxy)phenyl)-1H-imidazol-5-yl)(hydroxy)methyl)phenyl)butanoic acid

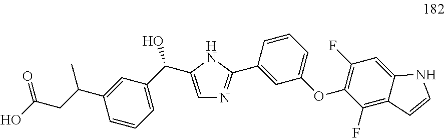 [SMILES: CC(CC(=O)O)c1cccc([C@H](O)c2cnc(-c3cccc(Oc4c(F)cc5[nH]ccc5c4F)c3)[nH]2)c1]

The title compound was similarly prepared in eight steps as described from Step A through H in Example 181, but exchanging 3-iodobenzaldehyde for 1-(3-bromophenyl)ethan-1-one and ethyl 2-(diethoxyphosphoryl)propanoate for ethyl 2-(diethoxyphosphoryl)acetate. MS: 504 m/z [M+H]⁺. ¹H NMR (500 MHz, CD₃OD) δ 7.56 (d, J=9.5 Hz, 1H), 7.48 (s, 1H), 7.34 (t, J=20.0, 10.0 Hz, 2H), 7.23-7.31 (m, 3H), 7.21 (br s, 1H), 7.16 (d, J=12.5 Hz, 1H), 6.93 (m, 1H), 6.75 (s, 1H), 6.57 (d, J=4.0 Hz, 1H), 5.79 (s, 1H), 3.27 (m, 1H), 2.58 (m, 2H), 1.31 (d, J=9.0 Hz, 3H) ppm.

### Example 183

### 3-(3-((2-(3-((4-Ethyl-1H-indol-5-yl)oxy)phenyl)-1H-imidazol-5-yl)(hydroxy)methyl)phenyl)propanoic acid

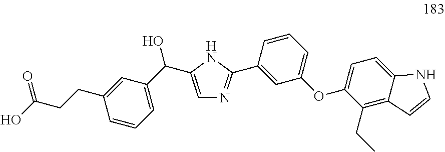 [SMILES: CCc1c(Oc2cccc(-c3ncc(C(O)c4cccc(CCC(=O)O)c4)[nH]3)c2)ccc2[nH]ccc12]

### A. Methyl 3-(3-(hydroxy(2-(3-((4-vinyl-1H-indol-5-yl)oxy)phenyl)-1H-imidazol-5-yl)methyl)phenyl)propanoate

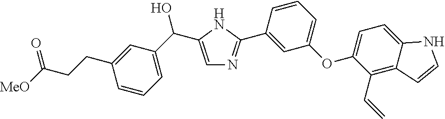 [SMILES: C=Cc1c(Oc2cccc(-c3ncc(C(O)c4cccc(CCC(=O)OC)c4)[nH]3)c2)ccc2[nH]ccc12]

Exchanging methyl 2-fluoro-3-(3-(3-formyloxiran-2-yl)phenyl)propanoate for methyl 3-(3-(2-formylcyclopropyl)phenyl)propanoate and 3-((4-methyl-1H-indol-5-yl)oxy)benzimidamide for 3-((4-vinyl-1H-indol-5-yl)oxy)benzimidamide (480 mg, 1.73 mmol), the reaction conditions described for Step J of Example 173 was used to prepare the title compound as a yellow solid (150 mg, 16%). MS: 494[M+H]⁺.

### B. Methyl 3-(3-((2-(3-((4-ethyl-1H-indol-5-yl)oxy)phenyl)-1H-imidazol-5-yl)(hydroxy)methyl)phenyl)propanoate

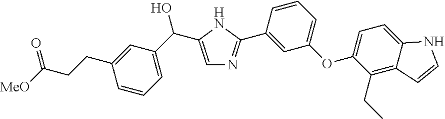 [SMILES: CCc1c(Oc2cccc(-c3ncc(C(O)c4cccc(CCC(=O)OC)c4)[nH]3)c2)ccc2[nH]ccc12]

A mixture of methyl 3-(3-(hydroxy(2-(3-((4-vinyl-1H-indol-5-yl)oxy)phenyl)-1H-imidazol-5-yl)methyl)phenyl)propanoate (60 mg, 0.12 mmol) and 10% Pd/C (20 mg) in methanol (7 mL) was stirred at room temperature under hydrogen atmosphere for three hours. The mixture was filtered through a pad of Celite, and the filtrate was concentrated to afford the title compound as yellow oil (51 mg, 85%). MS: 496 m/z [M+H]⁺.

### C. Example 183. 3-(3-((2-(3-((4-ethyl-1H-indol-5-yl)oxy)phenyl)-1H-imidazol-5-yl)(hydroxy)methyl)phenyl)propanoic acid

To a stirred solution of methyl 3-(3-((2-(3-((4-ethyl-1H-indol-5-yl)oxy)phenyl)-1H-imidazol-5-yl)(hydroxy)methyl)phenyl)propanoate (76 mg, 0.15 mmol) in THF (5 mL) was added a solution of lithium hydroxide (18 mg, 0.75 mmol) in water (3 mL). The reaction mixture was stirred at room temperature for two hours, quenched with 1 N hydrochloric acid (0.8 mL, 0.8 mmol) and extracted with ethyl acetate (20 mL×3). The combined organic extracts were washed with brine (20 mL), dried over sodium sulfate, filtered and concentrated. The resulting residue was purified by preparative HPLC to afford the title compound as a white solid (34 mg, 46%). MS: 482 m/z [M+H]⁺. ¹H NMR (400 MHz, CD₃OD) δ 7.48 (d, J=8.0 Hz, 1H), 7.42 (s, 1H), 7.35-7.26 (m, 6H), 7.18-7.15 (m, 1H), 6.83 (d, J=8.0 Hz, 1H), 6.81 (d, J=8.8 Hz, 1H), 6.75 (s, 1H), 6.54 (d, J=2.8 Hz, 1H), 5.78 (s, 1H), 2.93 (t, J=7.6 Hz, 2H), 2.85 (q, J=7.2 Hz, 2H), 2.59 (t, J=7.6 Hz, 2H), 1.22 (t, J=7.6 Hz, 3H) ppm.

### Example 184

### rac-(1r,2r)-2-(3-(1-(2-(3-((4,6-Difluoro-1H-indol-5-yl)oxy)phenyl)-1H-imidazol-4-yl)-1-hydroxyethyl)phenyl)cyclopropane-1-carboxylic acid

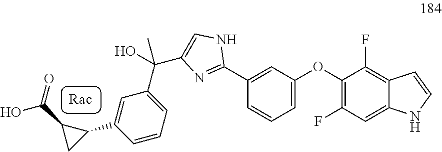 [SMILES: CC(O)(c1cccc([C@@H]2C[C@H]2C(=O)O)c1)c1c[nH]c(-c2cccc(Oc3c(F)cc4[nH]ccc4c3F)c2)n1]

### A. rac-Ethyl (1R,2R)-2-(3-bromophenyl)cyclopropane-1-carboxylate

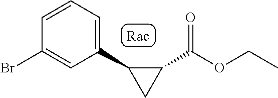

To a stirred solution of copper(II) acetylacetonate (156 mg, 0.6 mmol) in anhydrous dichloromethane (40 mL) was added 3-bromostyrene (2.62 mL, 20 mmol). The reaction mixture was stirred at 40° C. for five minutes and a solution of ethyl diazoacetate (3.12 mL, 30 mmol) in dichloromethane (40 mL) was added over five hours. After the addition, the mixture was stirred for one more hour, diluted with dichloromethane (100 mL) and washed with saturated sodium bicarbonate (60 mL×2) and brine (30 mL×2) solutions. The organic phase was dried over sodium sulfate, filtered and concentrated. The resulting residue was purified by silica gel chromatography (petroleum ether/ethyl acetate, v/v, 10/1) to afford the title compound as a colorless oil (2.75 g, 71%). ¹H NMR (400 MHz, CDCl₃) δ 7.35 (d, J=7.8 Hz, 1H), 7.25 (s, 1H), 7.16 (t, J=7.8 Hz, 1H), 7.05 (d, J=7.7 Hz, 1H), 4.23-4.16 (m, 2H), 2.49 (m, 1H), 1.90 (m, 1H), 1.62 (m, 1H), 1.30 (m, 4H) ppm.

### B. rac-Ethyl (1r,2r)-2-(3-((E)-4-((tert-butyldimethylsilyl)oxy)but-2-en-2-yl)phenyl)cyclopropane-1-carboxylate

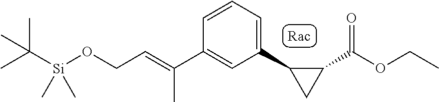 [SMILES: CCOC(=O)[C@@H]1C[C@H]1c1cccc(/C(C)=C/CO[Si](C)(C)C(C)(C)C)c1]

A mixture of rac-ethyl (1r,2r)-2-(3-bromophenyl)cyclopropane-1-carboxylate (800 mg, 2.98 mmol), tert-butyldimethyl(3-(4,4,5,5-tetramethyl-1,3,2-dioxaborolan-2-yl)but-2-enyloxy)silane (1.12 g, 3.58 mmol), Pd(dppf)Cl₂ (243 mg, 0.298 mmol) and sodium carbonate (633 mg, 5.97 mmol) in dimethoxyethane (50 mL) and water (5 mL) was heated at 80° C. for four hours. The reaction mixture was then cooled to room temperature, diluted with dichloromethane (50 mL), washed with saturated sodium bicarbonate (10 mL×3) solution, dried over sodium sulfate, and concentrated. The resulting residue was purified by silica gel chromatography (10% ethyl acetate in petroleum ether) to afford the title compound as a colorless oil (1 g, 89%). MS: 397 m/z [M+Na]⁺.

### C. rac-Ethyl (1R,2R)-2-(3-((E)-4-hydroxybut-2-en-2-yl)phenyl)cyclopropane-1-carboxylate

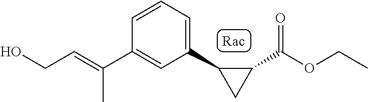 [SMILES: CCOC(=O)[C@@H]1C[C@H]1c1cccc(/C(C)=C/CO)c1]

To a stirred solution of rac-ethyl (1r,2r)-2-(3-((E)-4-((tert-butyldimethylsilyl)oxy)but-2-en-2-yl)phenyl)cyclopropane-1-carboxylate (500 mg, 1.08 mmol) in THF (3 mL) was added tetrabutylammonium fluoride (1M in THF) (8.0 mL, 8.0 mmol) at room temperature. The reaction mixture was stirred at room temperature for one hour, extracted with dichloromethane (30 mL×3), dried over sodium sulfate and concentrated. The resulting residue was purified by flash chromatography over silica (1-5% methanol in dichloromethane) to afford the title compound as a yellow oil (260 mg, 84%). MS: 283 m/z [M+Na]⁺.

### D. rac-Ethyl (1r,2r)-2-(3-(3-(hydroxymethyl)-2-methyloxiran-2-yl)phenyl)cyclopropane-1-carboxylate

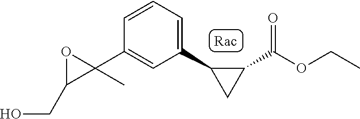 [SMILES: CCOC(=O)[C@@H]1C[C@H]1c1cccc(C2(C)OC2CO)c1]

To a stirred solution of rac-ethyl (1r,2r)-2-(3-((E)-4-hydroxybut-2-en-2-yl)phenyl)cyclopropane-1-carboxylate (380 mg, 1.46 mmol) and 1,1,1-trifluoropropan-2-one (980 mg, 8.77 mmol) in acetonitrile (10 mL) was added a solution of Oxone (1.79 g, 2.92 mmol) and sodium bicarbonate (490 mg, 5.85 mmol) in water (10 mL) at 0° C. The reaction mixture was warmed up to room temperature, stirred for 30 minutes and extracted with dichloromethane (30 mL×3). The combined organic extracts were dried over sodium sulfate, filtered and concentrated. The resulting residue was purified by flash chromatography over silica to afford the title compound as yellow oil (380 mg, 94%). MS: 299 m/z [M+Na]⁺.

### E. rac-Ethyl (1r,2r)-2-(3-(3-formyl-2-methyloxiran-2-yl)phenyl)cyclopropane-1-carboxylate

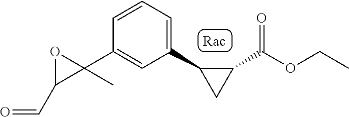 [SMILES: CCOC(=O)[C@@H]1C[C@H]1c1cccc(C2(C)OC2C=O)c1]

To a stirred solution of rac-ethyl (1r,2r)-2-(3-(3-(hydroxymethyl)-2-methyloxiran-2-yl)phenyl)cyclopropane-1-carboxylate (380 mg, 1.38 mmol) in dichloromethane (20 mL) were added in one portion BAIB (532 mg, 1.65 mmol) and TEMPO (43 mg, 0.27 mmol). The reaction mixture was then stirred at 25° C. for two hours and concentrated to dryness under reduced pressure at room temperature. The resulting residue was purified by flash chromatography over silica (petroleum ether/ethyl acetate, v/v, 3/1) to afford the title compound as a yellow oil (220 mg, 58%). ¹H NMR (400 MHz, CDCl₃) δ 9.58 (d, J=4.8 Hz, 1H), 7.30-7.04 (m, 4H), 4.17 (m, 2H), 3.30 (d, J=4.4 Hz, 1H), 2.54-2.49 (m, 1H), 1.92-1.88 (m, 1H), 1.82 (s, 3H), 1.63-1.56 (m, 1H), 1.34-1.26 (m, 4H) ppm

### F. rac-Ethyl (1r,2r)-2-(3-(1-(2-(3-((4,6-difluoro-1H-indol-5-yl)oxy)phenyl)-1H-imidazol-4-yl)-1-hydroxyethyl)phenyl)cyclopropane-1-carboxylate

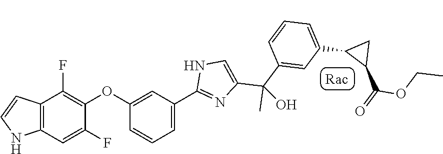

A mixture of rac-ethyl (1R,2R)-2-(3-(3-formyl-2-methyloxiran-2-yl)phenyl)cyclopropane-1-carboxylate (220 mg, 0.8 mmol) and 3-((4,6-difluoro-1H-indol-5-yl)oxy)benzimidamide (230 mg, 0.8 mmol, Intermediate 24-2) in dry DMF (12 mL) was stirred overnight at 80° C. under nitrogen and then concentrated to dryness. The resulting residue was purified by flash chromatography over silica (dichloromethane/methanol/conc.ammonia, v/v, 20/1/0.01) to afford the title compound as a white solid (280 mg, 45%). MS: 544 m/z [M+H]⁺.

### G. Example 184. rac-(1r,2r)-2-(3-(1-(2-(3-((4,6-Difluoro-1H-indol-5-yl)oxy)phenyl)-1H-imidazol-4-yl)-1-hydroxyethyl)phenyl)cyclopropane-1-carboxylic acid

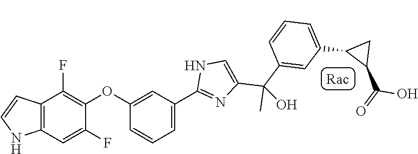

To a stirred solution of rac-ethyl (1r,2r)-2-(3-(1-(2-(3-((4,6-difluoro-1H-indol-5-yl)oxy)phenyl)-1H-imidazol-4-yl)-1-hydroxyethyl)phenyl)cyclopropane-1-carboxylate (65 mg, 0.12 mmol) in THF (6 mL) and water (3 mL) was added lithium hydroxide monohydrate (15 mg, 0.6 mmol). The reaction mixture was stirred at room temperature for four hours, concentrated to remove THF and acidified with 3N hydrochloric acid to pH˜3. The precipitate was collected by filtration and dried to afford the title compound as a white solid (44 mg, 71%). MS: 516 m/z [M+H]⁺. ¹H NMR (400 MHz, CD₃OD) δ 7.56 (d, J=7.6 Hz, 1H), 7.55-7.47 (m, 2H), 7.37 (s, 1H), 7.32-7.24 (m, 4H), 7.17-7.15 (d, J=10.0 Hz, 1H), 7.10-7.04 (m, 2H), 6.57-6.56 (d, J=3.2 Hz, 1H), 2.51-2.46 (m, 1H), 1.92 (s, 3H), 1.85-1.81 (m, 1H), 1.56-1.51 (m, 1H), 1.38-1.30 (m, 1H) ppm.

The following example were prepared utilizing similar procedures as described in Example 184.

| | Ex. | | | LCMS | MS m/z |
|---|---|---|---|---|---|
| Structure | No. | Name | ¹H NMR | Rₜ (min) | [M + H]⁺ |
| 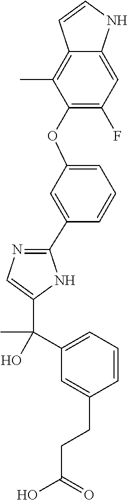 [SMILES: Cc1c(Oc2cccc(-c3ncc(C(C)(O)c4cccc(CCC(=O)O)c4)[nH]3)c2)c(F)cc2[nH]ccc12] | 185 | 3-(3-(1-(2-(3-((6- fluoro-4-methyl-1H- indol-5- yl)oxy)phenyl)-1H- imidazol-5-yl)-1- hydroxyethyl)phenyl) propanoic acid | (400 MHz, CD₃OD) δ 7.49 (d, J = 7.6 Hz, 1H), 7.39 (s, 2H), 7.34 (t, J = 8.0 Hz, 1H), 7.30-7.21 (m, 3H), 7.13 (d, J = 5.6 Hz, 1H), 7.11 (d, J = 10.4 Hz, 1H), 6.88 (s, 1H), 6.82 (dd, J = 1.2, 8.0 Hz, 1H), 6.52 (d, J = 3.2 Hz, 1H), 2.91 (t, J = 7.6 Hz, 2H), 2.56 (t, J = 7.6 Hz, 2H), 2.38 (s, 3H), 1.88 (s, 3H) ppm | 0.85 | 500 |
| 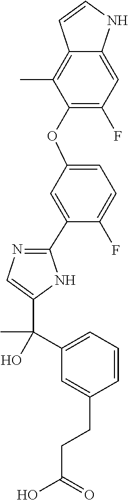 [SMILES: Cc1c(Oc2ccc(F)c(-c3ncc(C(C)(O)c4cccc(CCC(=O)O)c4)[nH]3)c2)c(F)cc2[nH]ccc12] | 186 | 3-(3-(1-(2-(2-fluoro- 5-((6-fluoro-4- methyl-1H-indol-5- yl)oxy)phenyl)-1H- imidazol-5-yl)-1- hydroxyethyl)phenyl) propanoic acid | (400 MHz, CD₃OD) δ 7.27 (dd, J = 3.2, 6.0 Hz, 1H), 7.23 (s, 1H), 7.08-7.15 (m, 3H), 6.90-7.03 (m, 3H), 6.81 (s, 1H), 6.74 (dt, J = 8.8, 3.6 Hz, 1H), 6.39 (d, J = 3.2 Hz, 1H), 2.79 (t, J = 7.6 Hz, 2H), 2.44 (t, J = 7.6 Hz, 2H), 2.26 (s, 3H), 1.74 (s, 3H) ppm | 0.87 | 518 |
| 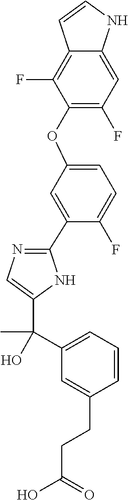 [SMILES: CC(O)(c1cccc(CCC(=O)O)c1)c1cnc(-c2cc(Oc3c(F)cc4[nH]ccc4c3F)ccc2F)[nH]1] | 187 | 3-(3-(1-(2-(5-((4,6- difluoro-1H-indol-5- yl)oxy)-2- fluorophenyl)-1H- imidazol-5-yl)-1- hydroxyethyl)phenyl) propanoic acid | (400 MHz, CD₃OD) δ 7.50 (m, 1H), 7.37 (s, 1H), 7.32-7.10 (m, 6H), 7.01- 6.92 (m, 2H), 6.56 (d, J = 2.8 Hz, 1H), 2.91 (t, J = 7.6 Hz, 2H), 2.57 (t, J = 7.6 Hz, 2H), 1.88 (s, 3H) ppm | 0.86 | 522 |
| 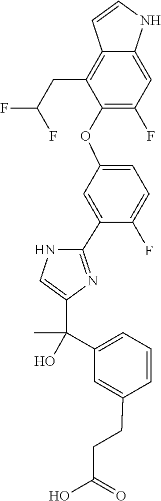 [SMILES: CC(O)(c1cccc(CCC(=O)O)c1)c1c[nH]c(-c2cc(Oc3c(F)cc4[nH]ccc4c3CC(F)F)ccc2F)n1] | 188 | 3-(3-(1-(2-(5-((4- (2,2-difluoroethyl)-6- fluoro-1H-indol-5- yl)oxy)-2- fluorophenyl)-1H- imidazol-4-yl)-1- hydroxyethyl)phenyl) propanoic acid | (400 MHz, CD₃OD) δ 7.50 (dd, J = 2.8, 5.6 Hz, 1H), 7.38 (s, 1H), 7.34 (d, J = 3.2 Hz, 1H) , 7.30-7.19 (m, 4H) , 7.13 (d, J = 7.2 Hz, 1H) , 7.04 (s, 1H), 6.97-6.93 (dt, J = 3.6, 8.8 Hz, 1H), 6.59 (d, J = 2.8 Hz, 1H) , 6.10 (tt, J = 4.8, 57.2 Hz, 1H), 3.42 (td, J = 16.4, 4.8 Hz, 2H), 2.91 (t, J = 7.6 Hz, 2H), 2.58 (t, J = 7.6 Hz, 2H), 1.89 (s, 3H) ppm | 0.87 | 568 |
| 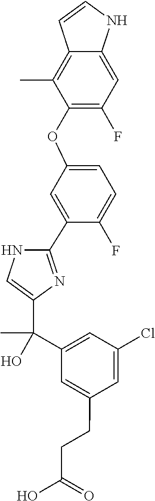 [SMILES: Cc1c(Oc2ccc(F)c(-c3nc(C(C)(O)c4cc(Cl)cc(CCC(=O)O)c4)c[nH]3)c2)c(F)cc2[nH]ccc12] | 189 | 3-(3-chloro-5-(1-(2- (2-fluoro-5-((6- fluoro-4-methyl-1H- indol-5- yl)oxy)phenyl)-1H- imidazol-4-yl)-1- hydroxyethyl)phenyl) propanoic acid | (400 MHz, CD₃OD) δ 7.41 (dd, J = 2.8, 5.6 Hz, 1H), 7.34 (s, 1H), 7.28-7.27 (m, 2H), 7.18-7.10 (m, 3H), 6.99 (s, 1H), 6.87 (dt, J = 8.8, 3.6 Hz, 1H), 6.52 (d, J = 3.2 Hz, 1H), 2.88 (t, J = 7.6 Hz, 2H), 2.57 (t, J = 7.6 Hz, 2H), 2.39 (s, 3H), 1.86 (s, 3H) ppm | 0.99 | 552 |
| 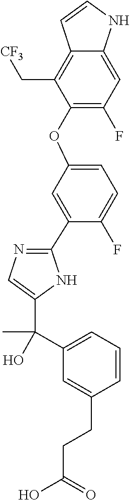 [SMILES: CC(O)(c1cccc(CCC(=O)O)c1)c1cnc(-c2cc(Oc3c(F)cc4[nH]ccc4c3CC(F)(F)F)ccc2F)[nH]1] | 190 | 3-(3-(1-(2-(2-fluoro- 5-((6-fluoro-4-(2,2,2- trifluoroethyl)-1H- indol-5- yl)oxy)phenyl)-1H- imidazol-5-yl)-1- hydroxyethyl)phenyl) propanoic acid | (400 MHz, CD₃OD) δ 7.44 (dd, J = 6.0, 3.2 Hz, 1H), 7.37-7.35 (m, 2H), 7.32- 7.11 (m, 5H), 6.95 (s, 1H), 6.93-6.88 (m, 1H), 6.58 (d, J = 2.8 Hz, 1H), 3.75 (q, J = 10.8 Hz, 2H), 2.91 (t, J = 7.6 Hz, 2H), 2.58 (t, J = 7.6 Hz, 2H), 1.88 (s, 3H) ppm. | 0.95 | 586 |
| 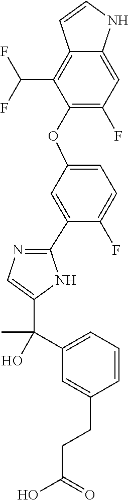 [SMILES: CC(O)(c1cccc(CCC(=O)O)c1)c1cnc(-c2cc(Oc3c(F)cc4[nH]ccc4c3C(F)F)ccc2F)[nH]1] | 191 | 3-(3-(1-(2-(5-((4- (difluoromethyl)-6- fluoro-1H-indol-5- yl)oxy)-2- fluorophenyl)-1H- imidazol-5-yl)-1- hydroxyethyl)phenyl) propanoic acid | (400 MHz, CD₃OD) δ 7.52-7.39 (m, 4H), 7.30-7.15 (m, 5H), 7.01 (s, 1H), 6.96-6.93 (m, 1H), 6.76 (s, 1H), 2.93 (t, J = 6.8 Hz, 2H), 2.61 (t, J = 6.8 Hz, 2H), 1.90 (s, 3H) ppm | 0.92 | 554 |
| 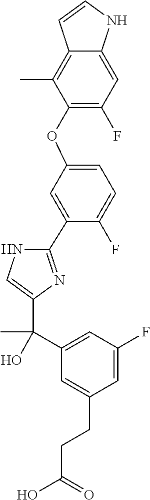 [SMILES: Cc1c(Oc2ccc(F)c(-c3nc(C(C)(O)c4cc(F)cc(CCC(=O)O)c4)c[nH]3)c2)c(F)cc2[nH]ccc12] | 192 | 3-(3-fluoro-5-(1-(2- (2-fluoro-5-((6- fluoro-4-methyl-1H- indol-5- yl)oxy)phenyl)-1H- imidazol-4-yl)-1- hydroxyethyl)phenyl) propanoic acid | (400 MHz, CD₃OD) δ 7.42 (m, 1H), 7.27 (d, J = 3.6 Hz, 1H), 7.18-7.04 (m, 4H), 6.97 (s, 1H), 6.88-6.85 (m, 2H), 6.52 (d, J = 3.2 Hz, 1H), 2.92 (t, J = 7.6 Hz, 2H), 2.59 (t, J = 7.6 Hz, 2H), 2.39 (s, 3H), 1.86 (s, 3H) ppm. | 0.94 | 536 |
| 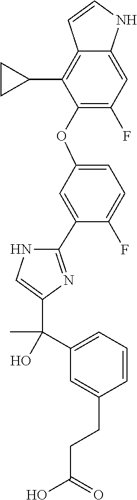 [SMILES: CC(O)(c1cccc(CCC(=O)O)c1)c1c[nH]c(-c2cc(Oc3c(F)cc4[nH]ccc4c3C3CC3)ccc2F)n1] | 193 | 3-(3-(1-(2-(5-((4- cyclopropyl-6-fluoro- 1H-indol-5-yl)oxy)- 2-fluorophenyl)-1H- imidazol-4-yl)-1- hydroxyethyl)phenyl) propanoic acid | (400 MHz, CD₃OD) δ 7.43-7.41 (m, 2H), 7.38-7.30 (m, 4H), 7.28-7.27 (m, 1H), 7.20-7.18 (m, 1H), 7.14-7.10 (m, 2H), 6.60 (dd, J = 0.8, 0.8 Hz, 1H), 2.93 (t, J = 7.6 Hz, 2H), 2.61 (t, J = 7.6 Hz, 2H), 2.04-2.00 (m, 1H), 1.95 (s, 3H), 1.02-0.98 (m, 2H), 0.93-0.88 (m, 2H) ppm | 0.93 | 544 |
| 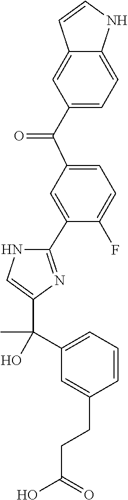 [SMILES: CC(O)(c1cccc(CCC(=O)O)c1)c1c[nH]c(-c2cc(C(=O)c3ccc4[nH]ccc4c3)ccc2F)n1] | 194 | 3-(3-(1-(2-(2-fluoro- 5-(1H-indole-5- carbonyl)phenyl)-1H- imidazol-4-yl)-1- hydroxyethyl)phenyl) propanoic acid | (400 MHz, CD₃OD) δ 8.41-8.37 (m, 1H), 8.10 (s, 1H), 7.87-7.84 (m, 1H), 7.71 (d, J = 8.4 Hz, 1H), 7.53 (d, J = 8.8 Hz, 1H), 7.38-7.44 (m, 3H), 7.31 (d, J = 8.0 Hz, 1H), 7.24 (t, J = 7.2 Hz, 1H), 7.12 (d, J = 6.8 Hz, 1H), 7.02 (br s, 1H), 6.62 (d, J = 3.2 Hz, 1H), 2.91 (t, J = 7.6 Hz, 2H), 2.59 (t, J = 7.6 Hz, 2H), 1.91 (s, 3H) ppm. | 0.74 | 498 |
| 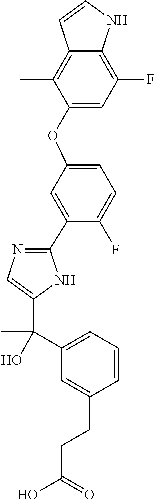 [SMILES: Cc1c(Oc2ccc(F)c(-c3ncc(C(C)(O)c4cccc(CCC(=O)O)c4)[nH]3)c2)cc(F)c2[nH]ccc12] | 195 | 3-(3-(1-(2-(2-fluoro- 5-((7-fluoro-4- methyl-1H-indol-5- yl)oxy)phenyl)-1H- imidazol-5-yl)-1- hydroxyethyl)phenyl) propanoic acid | (400 MHz,CD₃OD) δ 7.54 (m, 2H), 7.34 (d, J = 2.8 Hz, 1H), 7.30-7.16 (m, 3H), 7.13 (d, J = 7.6 Hz, 1H), 7.02 (s, 1H), 6.93-6.89 (m, 1H), 6.62-6.58 (m, 2H), 2.91 (t, J = 8.0 Hz, 2 H), 2.58 (t, J = 7.6 Hz, 2H), 2.32 (s, 3H), 1.89 (s, 3H) ppm | 0.88 | 518 |
| 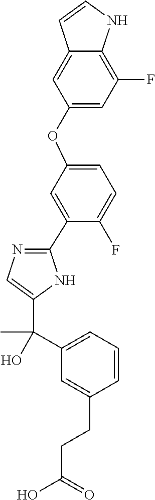 [SMILES: CC(O)(c1cccc(CCC(=O)O)c1)c1cnc(-c2cc(Oc3cc(F)c4[nH]ccc4c3)ccc2F)[nH]1] | 196 | 3-(3-(1-(2-(2-fluoro- 5-((7-fluoro-1H- indol-5- yl)oxy)phenyl)-1H- imidazol-5-yl)-1- hydroxyethyl)phenyl) propanoic acid | (400 MHz,CD₃OD) δ 7.54 (dd, J = 6.4, 3.2 Hz, 1H), 7.38 (s, 1H), 7.32 (d, J = 3.2 Hz, 1H), 7.28-7.18 (m, 3H), 7.12 (d, J = 7.2 Hz, 1H), 7.05 (d, J = 2.0 Hz, 1H), 7.02-6.99 (m, 1H), 6.97 (s, 1H), 6.87 (dd, J = 12.0, 2.0 Hz, 1H), 6.48 (t, J = 3.2 Hz, 1H), 2.91 (t, J = 7.6 Hz, 2H), 2.58 (t, J = 7.6 Hz, 2H), 1.89 (s, 3H) ppm | 0.83 | 504 |
| 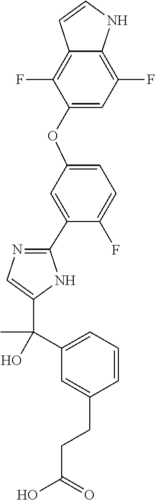 [SMILES: CC(O)(c1cccc(CCC(=O)O)c1)c1cnc(-c2cc(Oc3cc(F)c4[nH]ccc4c3F)ccc2F)[nH]1] | 197 | 3-(3-(1-(2-(5-((4,7- difluoro-1H-indol-5- yl)oxy)-2- fluorophenyl)-1H- imidazol-5-yl)-1- hydroxyethyl)phenyl) propanoic acid | (400 MHz, CD₃OD) δ 7.52-7.50 (m, 1H), 7.38-7.35 (m, 2H), 7.28-7.11 (m, 4H), 6.99-6.94 (m, 2H), 6.79-6.75 (m, 1H), 6.63-6.61 (m, 1H), 2.91 (t, J = 7.6 Hz, 2H), 2.56 (t, J = 8.0 Hz, 2H), 1.88 (s, 3H) ppm. | 0.84 | 522 |
| 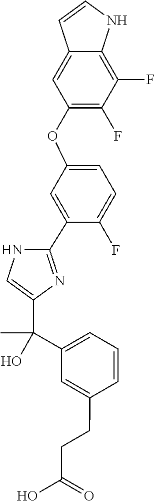 [SMILES: CC(O)(c1cccc(CCC(=O)O)c1)c1c[nH]c(-c2cc(Oc3cc4cc[nH]c4c(F)c3F)ccc2F)n1] | 198 | 3-(3-(1-(2-(5-((6,7- difluoro-1H-indol-5- yl)oxy)-2- fluorophenyl)-1H- imidazol-4-yl)-1- hydroxyethyl)phenyl) propanoic acid | (400 MHz, CD₃OD) δ 7.53 (m, 1H), 7.37-7.39 (m, 2H), 7.27-7.11 (m, 5H), 7.04-7.00 (m, 1H), 6.98 (s, 1H), 6.51 (m, 1H), 2.93 (t, J = 7.6 Hz, 2H), 2.65 (t, J = 7.6 Hz, 2H), 1.88 (s, 3H) ppm. | 0.84 | 522 |
| 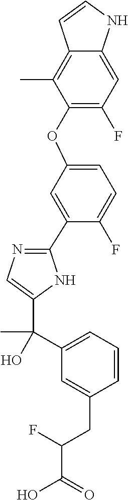 [SMILES: Cc1c(Oc2ccc(F)c(-c3ncc(C(C)(O)c4cccc(CC(F)C(=O)O)c4)[nH]3)c2)c(F)cc2[nH]ccc12] | 199 | 2-fluoro-3-(3-(1-(2- (2-fluoro-5-((6- fluoro-4-methyl-1H- indol-5- yl)oxy)phenyl)-1H- imidazol-5-yl)-1- hydroxyethyl)phenyl) propanoic acid | (400 MHz, CD₃OD) δ 7.39-7.42 (m, 2H), 7.30-7.33 (m, 1H), 7.22-7.27 (m, 2H), 7.14-7.20 (m, 2H), 7.09-7.12 (m, 1H), 7.015 (s, 1H), 6.85-6.90 (m, 1H), 6.51 (d, J = 3.2 Hz, 1H), 4.83-4.98 (m, 1H), 2.99-3.25 (m, 2H), 2.383 (s, 3H), 1.885 (s, 3H) ppm. | 0.86 | 536 |
| 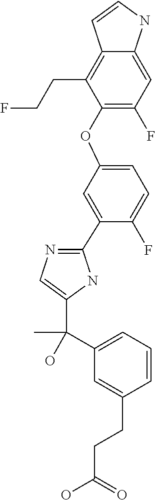 [SMILES: CC(O)(c1cccc(CCC(=O)O)c1)c1cnc(-c2cc(Oc3c(F)cc4[nH]ccc4c3CCF)ccc2F)[nH]1] | 200 | 3-(3-(1-(2-(2-fluoro- 5-((6-fluoro-4-(2- fluoroethyl)-1H- indol-5- yl)oxy)phenyl)-1H- imidazol-5-yl)-1- hydroxyethyl)phenyl) propanoic acid | (400 MHz, CD₃OD) δ 7.43 (dd, J = 6.0, 3.2 Hz, 1H), 7.35 (s, 1H) 7.30-7.08 (m, 6H), 6.94 (s, 1H), 6.89-6.85 (m, 1H ), 6.55 (d, J = 3.2 Hz, 1H), 4.59 (dt, J = 47.2, 6.8 Hz, 2H), 3.30-3.20 (m, 2H), 2.89 (t, J = 7.6 Hz, 2H), 2.56 (t, J = 7.6 Hz, 2H), 1.86 (s, 3H) | 0.85 | 550 |
| 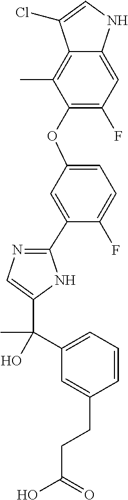 [SMILES: Cc1c(Oc2ccc(F)c(-c3ncc(C(C)(O)c4cccc(CCC(=O)O)c4)[nH]3)c2)c(F)cc2[nH]cc(Cl)c12] | 201 | 3-(3-(1-(2-(5-((3- chloro-6-fluoro-4- methyl-1H-indol-5- yl)oxy)-2- fluorophenyl)-1H- imidazol-5-yl)-1- hydroxyethyl)phenyl) propanoic acid | 400 MHz, CD₃OD) δ 7.39 (dd, J = 6.4, 3.2 Hz, 1H), 7.37 (s, 1H), 7.30-7.08 (m, 6H), 6.95 (s, 1H), 6.93-6.87 (m, 1H), 2.91 (t, J = 7.6 Hz, 2H), 2.63 (s, 3H), 2.58 (t, J = 7.6 Hz, 2H), 1.88 (s, 3H). | 0.94 | 552 |
| 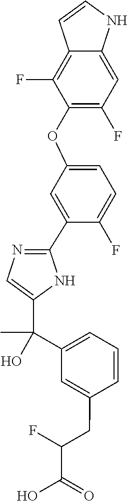 [SMILES: CC(O)(c1cccc(CC(F)C(=O)O)c1)c1cnc(-c2cc(Oc3c(F)cc4[nH]ccc4c3F)ccc2F)[nH]1] | 202 | 3-(3-(1-(2-(5-((4,6- difluoro-1H-indol-5- yl)oxy)-2- fluorophenyl)-1H- imidazol-5-yl)-1- hydroxyethyl)phenyl)- 2-fluoropropanoic acid | (400 MHz, CD₃OD) δ 7.35-7.36 (m, 1H), 7.31 (d, J = 6.0 Hz, 1H), 7.22-7.26 (m, 1H), 7.13-7.20 (m, 3H), 6.97-7.08 (m, 4H), 6.44 (d, J = 3.2 Hz, 1H), 4.80- 4.83 (m, 1H), 2.92-3.16 (m, 2H), 1.79 (s, 3H) ppm. | 0.84 | 540 |
| 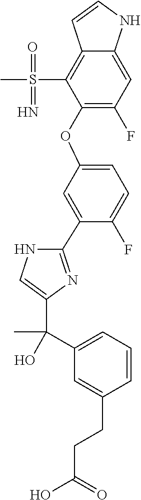 [SMILES: CC(O)(c1cccc(CCC(=O)O)c1)c1c[nH]c(-c2cc(Oc3c(F)cc4[nH]ccc4c3S(C)(=N)=O)ccc2F)n1] | 203 | 3-(3-(1-(2-(2-fluoro- 5-((6-fluoro-4-(S- methylsulfonimidoyl)- 1H-indol-5- yl)oxy)phenyl)-1H- imidazol-4-yl)-1- hydroxyethyl)phenyl) propanoic acid | (400 MHz, CD₃OD δ 7.60 (d, J = 10.5 Hz, 1H), 7.49 (d, J = 3.4 Hz, 1H), 7.40- 7.32 (m, 2H), 7.29-7.17 (m, 4H), 7.13- 7.04 (m, 2H), 6.94 (d, J = 12.0 Hz, 1H), 3.40 (s, 3H), 2.89 (t, J = 8.2 Hz, 2H), 2.54 (t, J = 8.2 Hz, 2H), 1.85 (s, 3H) ppm. | 0.64 | 581 |
| 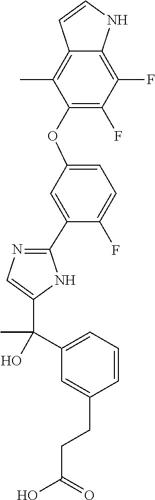 [SMILES: Cc1c(Oc2ccc(F)c(-c3ncc(C(C)(O)c4cccc(CCC(=O)O)c4)[nH]3)c2)c(F)c(F)c2[nH]ccc12] | 204 | 3-(3-(1-(2-(5-((6,7- difluoro-4-methyl- 1H-indol-5-yl)oxy)- 2-fluorophenyl)-1H- imidazol-5-yl)-1- hydroxyethyl)phenyl) propanoic acid | (400 MHz, CD₃OD δ 7.39-7.37 (m, 2H), 7.35 (d, J = 2.8 Hz, 1H), 7.31-7.24 (m, 3H), 7.17 (s, 1H), 7.15 (d, J = 7.2, 1H), 7.04 (dt, J = 8.8, 3.6 Hz, 1H), 6.59 (t, J = 3.2 Hz, 1H), 2.92 (t, J = 7.6 Hz, 2H), 2.59 (t, J = 7.6 Hz, 2H), 2.35 (s, 3H), 1.91 (s , 3H) ppm. | 0.9 | 536 |
| 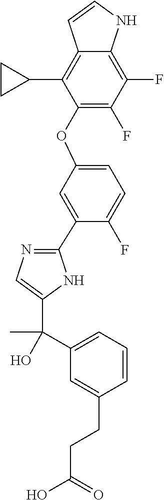 [SMILES: CC(O)(c1cccc(CCC(=O)O)c1)c1cnc(-c2cc(Oc3c(F)c(F)c4[nH]ccc4c3C3CC3)ccc2F)[nH]1] | 205 | 3-(3-(1-(2-(5-((4- cyclopropyl-6,7- difluoro-1H-indol-5- yl)oxy)-2- fluorophenyl)-1H- imidazol-5-yl)-1- hydroxyethyl)phenyl) propanoic acid | (400 MHz, CD₃OD δ 7.45 (dd, J = 6.0, 3.2 Hz, 1H), 7.37 (s, 1H), 7.33 (d, J = 3.2 Hz, 1H), 7.30-7.10 (m, 4H), 6.96- 6.85 (m, 2H), 6.67 (t, J = 3.2 Hz, 1H), 2.91 (t, J = 7.8 Hz, 2H), 2.57 (t, J = 7.8 Hz, 2H), 1.99-1.84 (m, 4H), 0.89 (dd, J = 15.9, 7.2 Hz, 4H) ppm. | 0.96 | 562 |
| 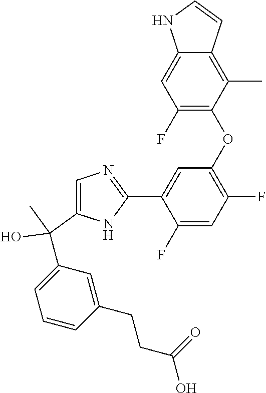 [SMILES: Cc1c(Oc2cc(-c3ncc(C(C)(O)c4cccc(CCC(=O)O)c4)[nH]3)c(F)cc2F)c(F)cc2[nH]ccc12] | 206 | 3-(3-(1-(2-(2,4- difluoro-5-((6-fluoro- 4-methyl-1H-indol-5- yl)oxy)phenyl)-1H- imidazol-5-yl)-1- hydroxyethyl)phenyl) propanoic acid | (400 MHz, CD₃OD δ 7.36 (t, J = 10.4 Hz, 1H), 7.31-7.29 (m, 2H), 7.25-7.10 (m, 5 H), 7.07 (s, 1H), 6.54 (d, J = 2.8 Hz, 1H), 2.87 (t, J = 8.0 Hz, 2H), 2.54 (t, J = 8.4 Hz, 2H), 2.43 (s, 3H), 1.83 (s, 3H) ppm. | 0.92 | 536 |
| 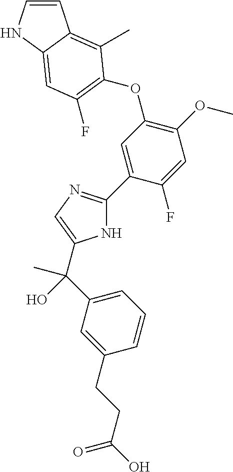 [SMILES: COc1cc(F)c(-c2ncc(C(C)(O)c3cccc(CCC(=O)O)c3)[nH]2)cc1Oc1c(F)cc2[nH]ccc2c1C] | 281 | 3-(3-(1-(2-(2-fluoro- 5-((6-fluoro-4- methyl-1H-indol-5- yl)oxy)-4- methoxyphenyl)-1H- imidazol-5-yl)-1- hydroxyethyl)phenyl) propanoic acid | (400 MHz, CD₃OD δ 7.30 (s, 1H), 7.27 (d, J = 2.8 Hz, 1H), 7.18-7.23 (m, 2H), 7.08-7.12 (m, 3H), 6.95-6.99 (m, 2H), 6.51 (d, J = 3.2 Hz, 1H), 4.03 (s, 3H), 2.87 (m, 2H), 2.54 (m, 2H), 2.38 (s, 3H), 1.82 (s, 3H) ppm. | 0.84 | 548 |
| 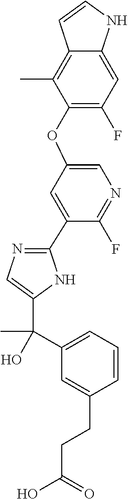 [SMILES: Cc1c(Oc2cnc(F)c(-c3ncc(C(C)(O)c4cccc(CCC(=O)O)c4)[nH]3)c2)c(F)cc2[nH]ccc12] | 207 | 3-(3-(1-(2-(2-fluoro- 5-((6-fluoro-4- methyl-1H-indol-5- yl)oxy)pyridin-3-yl)- 1H-imidazol-5-yl)-1- hydroxyethyl)phenyl) propanoic acid | (400 MHz, CD₃OD δ 7.91-7.87 (m, 2H), 7.36 (s, 1H), 7.31-7.21 (m, 3H), 7.16- 7.11 (m, 2H), 7.07 (s, 1H), 6.54 (d, J = 3.2 Hz, 1 H), 2.89 (t, J = 7.6 Hz, 2H), 2.57 (t, J = 7.6 Hz, 2H), 2.43 (s, 3H), 1.87 (s, 3 H) ppm | 0.92 | 519 |
| 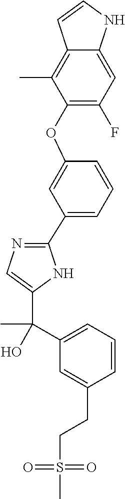 [SMILES: Cc1c(Oc2cccc(-c3ncc(C(C)(O)c4cccc(CCS(C)(=O)=O)c4)[nH]3)c2)c(F)cc2[nH]ccc12] | 208 | 1-(2-(3-((6-fluoro-4- methyl-1H-indol-5- yl)oxy)phenyl)-1H- imidazol-5-yl)-1-(3- (2- (methylsulfonyl)ethyl) phenyl)ethan-1-ol | (400 MHz, CD₃OD δ 7.45-7.49 (m, 2H), 7.27-7.39 (m, 5H), 7.18-7.20 (m, 1H), 7.10 (d, J = 10.8 Hz, 1H), 6.80-6.94 (m, 2H), 6.52 (d, J = 2.8 Hz, 1H), 3.40 (t, J = 8.0 Hz, 2H), 3.11 (t, J = 6.0 Hz, 2H), 2.89 (s, 3H), 2.38 (s, 3H), 1.89 (s, 3H) ppm | 0.84 | 534 |
| 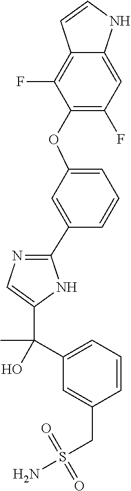 [SMILES: CC(O)(c1cccc(CS(N)(=O)=O)c1)c1cnc(-c2cccc(Oc3c(F)cc4[nH]ccc4c3F)c2)[nH]1] | 209 | (3-(1-(2-(3-((4,6- difluoro-1H-indol-5- yl)oxy)phenyl)-1H- imidazol-5-yl)-1- hydroxyethyl)phenyl) methanesulfonamide | | 0.77 | 525 |

### Example 210

### (Z)-5-(3-(1-(2-(3-((4,6-Difluoro-1H-indol-5-yl)oxy)phenyl)-1H-imidazol-5-yl)-1-hydroxyethyl)benzylidene)thiazolidine-2,4-dione

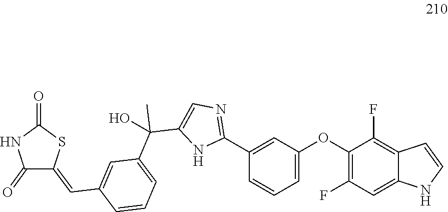

### A. 1-(3-(((tert-Butyldimethylsilyl)oxy)methyl)phenyl)-1-(2-(3-((4,6-difluoro-1H-indol-5-yl)oxy)phenyl)-1H-imidazol-5-yl)ethan-1-ol

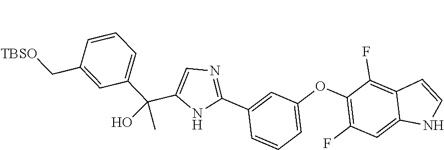

A mixture of 3-(3-(((tert-butyldimethylsilyl)oxy)methyl)phenyl)-3-methyloxirane-2-carbaldehyde (1.4 g, 4.88 mmol, Intermediate 26-3) and 3-((4,6-difluoro-1H-indol-5-yl)oxy)benzimidamide (1.5 g, 4.88 mmol, Intermediate 24-2) in dry DMF (15 mL) was purged with nitrogen and heated at 75° C. for eighteen hours. The solvent was evaporated and the residue was purified by flash chromatography over silica to afford the title compound as a yellow solid (1 g, 36%). MS-ESI m/z: 576 m/z [M+H]⁺.

### B. 1-(2-(3-((4,6-Difluoro-1H-indol-5-yl)oxy)phenyl)-1H-imidazol-5-yl)-1-(3-(hydroxymethyl)phenyl)ethan-1-ol

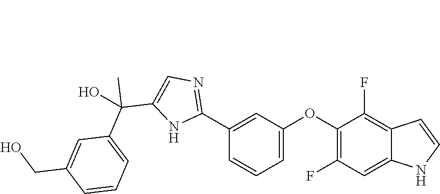

To a stirred solution of 1-(3-(((tert-butyldimethylsilyl)oxy)methyl)phenyl)-1-(2-(3-((4,6-difluoro-1H-indol-5-yl)oxy)phenyl)-1H-imidazol-5-yl)ethan-1-ol (1.5 g, 2.6 mmol) in THF (15 mL) was added tetrabutylammonium fluoride (1M in THF, 5.2 mL) dropwise. The mixture was stirred at room temperature for two hours, diluted with water (25 mL) and extracted with ethyl acetate (50 mL×2). The combined organic extracts were washed with brine (25 mL), dried over sodium sulfate and concentrated. The resulting residue was purified by flash chromatography over silica to afford the title compound as a yellow solid (620 mg, 52%). MS: 462 m/z [M+H]⁺.

### C. 3-(1-(2-(3-((4,6-Difluoro-1H-indol-5-yl)oxy)phenyl)-1H-imidazol-5-yl)-1-hydroxyethyl)benzaldehyde

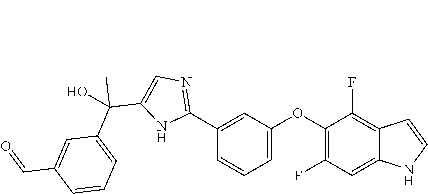

To a stirred solution of 1-(2-(3-((4,6-difluoro-1H-indol-5-yl)oxy)phenyl)-1H-imidazol-5-yl)-1-(3-(hydroxymethyl)phenyl)ethan-1-ol (460 mg, 1 mmol) in THF (20 mL) was added manganese dioxide (870 mg, 10 mmol). The reaction mixture was stirred at room temperature overnight, filtered and the filter cake was washed with THF (30 mL). The filtrate was concentrated and the resulting residue purified by flash chromatography over silica to afford the title compound as a yellow solid (360 mg, 78%). MS: 460 m/z [M+H]⁺.

### D. Example 210. (Z)-5-(3-(1-(2-(3-((4,6-Difluoro-1H-indol-5-yl)oxy)phenyl)-1H-imidazol-5-yl)-1-hydroxyethyl)benzylidene)thiazolidine-2,4-dione

A mixture of 3-(1-(2-(3-((4,6-difluoro-1H-indol-5-yl)oxy)phenyl)-1H-imidazol-5-yl)-1-hydroxyethyl)benzaldehyde (150 mg, 0.327 mmol), 2,4-thiazolidinedione (76 mg, 0.654 mmol) and tetrabutylammonium acetate (600 mg) was heated at 120° C. for thirty minutes and cooled to room temperature. Another portion of 2,4-thiazolidinedione (38 mg, 0.327 mmol) was added, and the mixture was stirred at 120° C. for twenty minutes. The mixture was dissolved in a mixture of ethyl acetate and methanol (50 mL, v/v, 5/1), washed with water (20 mL×2), brine, dried over sodium sulfate and concentrated. The resulting residue was purified by flash chromatography over silica to afford the title compound as a yellow solid (80 mg, 44%). MS-ESI m/z: 559 m/z [M+H]⁺. ¹H NMR (400 MHz, CD₃OD) δ 7.78 (s, 1H), 7.69 (s, 1H), 7.60-7.54 (m, 2H), 7.48-7.43 (m, 3H), 7.38 (t, J=8 Hz, 1H), 7.29 (d, J=2.8 Hz, 1H), 7.13 (d, J=10 Hz, 1H), 7.02 (s, 1H), 6.94-6.91 (dd, J=2, 8 Hz, 1H), 6.55 (d, J=3.2 Hz, 1H), 1.92 (s, 3H) ppm.

### Example 211

### 5-(3-(1-(2-(3-((4,6-Difluoro-1H-indol-5-yl)oxy)phenyl)-1H-imidazol-5-yl)-1-hydroxyethyl)benzyl)thiazolidine-2,4-dione

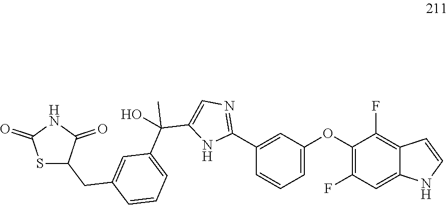

To a stirred solution of (Z)-5-(3-(1-(2-(3-((4,6-difluoro-1H-indol-5-yl)oxy)phenyl)-1H-imidazol-5-yl)-1-hydroxyethyl)benzylidene)thiazolidine-2,4-dione (130 mg, 0.232 mmol) in methanol (10 mL) was added 10% Pd/C (100 mg). The reaction mixture was purged with hydrogen and stirred at room temperature for eighteen hours under a hydrogen balloon. The catalyst was filtered through a pad of Celite and the filter cake was washed with methanol (25 mL). The filtrate was evaporated and the resulting residue was purified by flash chromatography over silica to afford the title compound as a grey solid (60 mg, 46%). MS: 561 m/z [M+H]⁺. ¹H NMR (400 MHz, CD₃OD) δ 7.56-7.53 (d, J=7.2 Hz 1H), 7.48 (s, 1H), 7.43-7.35 (m, 3H), 7.31-7.26 (m, 2H), 7.17-7.15 (t, J=8 Hz, 2H), 6.92 (d, J=8 Hz, 1H), 6.86 (s, 1H), 6.56-6.55 (d, J=3.2 Hz, 1H), 4.71-4.68 (m, 1H), 3.49-3.43 (m, 1H), 3.18-3.12 (m, 1H), 1.89 (s, 3H) ppm.

### Example 212

### N-((3-(1-(2-(3-((4,6-Difluoro-1H-indol-5-yl)oxy)phenyl)-1H-imidazol-5-yl)-1-hydroxyethyl)benzyl)sulfonyl)acetamide

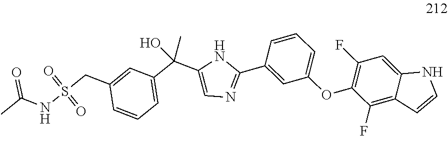

A mixture of (3-(1-(2-(3-((4,6-difluoro-1H-indol-5-yl)oxy)phenyl)-1H-imidazol-5-yl)-1-hydroxyethyl)phenyl)methanesulfonamide (Example 209, 26 mg, 0.05 mmol), acetic acid (3 mg, 0.05 mmol), TBTU (15 mg, 0.05 mmol), lithium chloride (1 mg, 0.02 mmol) and triethylamine (15 mg, 0.15 mmol) in THF (1 mL) was stirred at room temperature for sixteen hours, diluted with water (20 mL) and extracted with ethyl acetate (25 mL×4). The combined organic extracts were dried over sodium sulfate and concentrated. The resulting residue was purified by flash chromatography over silica (1-5% methanol in dichloromethane), followed by preparative HPLC to afford the title compound as a yellow solid (17 mg, 61%). LC-MS retention time, 0.81 min. MS-ESI m/z: 567 m/z [M+H]⁺.

### Example 213

### 3-(3-(1-(2-(2-Fluoro-5-((6-fluoro-4-methyl-1H-indol-5-yl)oxy)phenyl)-1H-imidazol-5-yl)ethyl)phenyl)propanoic acid

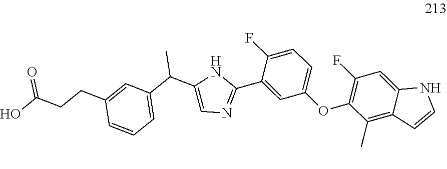

### A. Methyl 3-(3-(1-(2-(2-fluoro-5-((6-fluoro-4-methyl-1H-indol-5-yl)oxy)phenyl)-1H-imidazol-5-yl)-1-hydroxyethyl)phenyl)propanoate

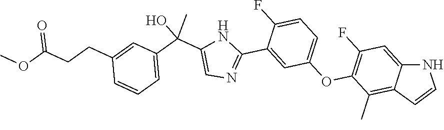

Exchanging methyl 2-fluoro-3-(3-(3-formyloxiran-2-yl)phenyl)propanoate for methyl 3-(3-(3-formyl-2-methyloxiran-2-yl)phenyl)propanoate (692 mg, 2.79 mmol, Intermediate 26) and 3-((4-methyl-1H-indol-5-yl)oxy)benzimidamide for 2-fluoro-5-((6-fluoro-4-methyl-1H-indol-5-yl)oxy)benzimidamide (700 mg, 2.33 mmol, Intermediate 24-10), the reaction conditions described for Step J of Example 172 was used to prepare the title compound as a light yellow solid (570 mg, 46%). MS: 532 m/z [M+H]⁺.

### B. Methyl 3-(3-(1-(2-(2-fluoro-5-((6-fluoro-4-methyl-1H-indol-5-yl)oxy)phenyl)-1H-imidazol-5-yl)vinyl)phenyl)propanoate

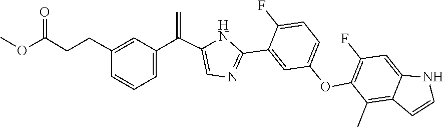 [SMILES: C=C(c1cccc(CCC(=O)OC)c1)c1cnc(-c2cc(Oc3c(F)cc4[nH]ccc4c3C)ccc2F)[nH]1]

A solution of methyl 3-(3-(1-(2-(2-fluoro-5-((6-fluoro-4-methyl-1H-indol-5-yl)oxy)phenyl)-1H-imidazol-5-yl)-1-hydroxyethyl)phenyl)propanoate (250 mg, 0.47 mmol) in formic acid (5 mL) was heated at 130° C. for forty minutes in a microwave synthesizer under nitrogen atmosphere. The mixture was concentrated to afford the title compound as a yellow solid (240 mg, 98%), which was used in the next step without further purification. MS: 514 m/z [M+H]⁺.

### C. Methyl 3-(3-(1-(2-(2-fluoro-5-((6-fluoro-4-methyl-1H-indol-5-yl)oxy)phenyl)-1H-imidazol-5-yl)ethyl)phenyl)propanoate

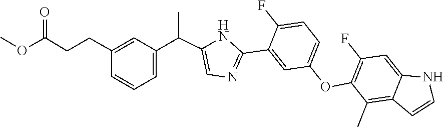 [SMILES: COC(=O)CCc1cccc(C(C)c2cnc(-c3cc(Oc4c(F)cc5[nH]ccc5c4C)ccc3F)[nH]2)c1]

A mixture of methyl 3-(3-(1-(2-(2-fluoro-5-((6-fluoro-4-methyl-1H-indol-5-yl)oxy)phenyl)-1H-imidazol-5-yl)vinyl)phenyl)propanoate (110 mg, 0.21 mmol) and 10% Pd/C (60 mg) in THF (20 mL) was stirred under hydrogen atmosphere at room temperature for five hours and then filtered through a pad of Celite to remove the catalyst. The filtrate was concentrated and the resulting residue was purified by preparative HPLC to afford the title compound as a white solid (40 mg, 36%). MS: 516 m/z [M+H]⁺. ¹H NMR (400 MHz, CD₃OD) δ 7.40-7.38 (m, 1H), 7.27 (d, J=3.2 Hz, 1H), 7.22-7.07 (m, 5H), 7.05-7.03 (m, 1H), 6.85-6.82 (m, 2H), 6.51 (d, J=3.2 Hz, 1H), 4.11 (q, J=7.2 Hz, 1H), 3.60 (s, 3H), 2.88 (t, J=7.6 Hz, 2H), 2.60 (t, J=7.6 Hz, 2H), 2.38 (s, 3H), 1.58 (d, J=7.2 Hz, 3H) ppm.

### D. Example 213. 3-(3-(1-(2-(2-Fluoro-5-((6-fluoro-4-methyl-1H-indol-5-yl)oxy)phenyl)-1H-imidazol-5-yl)ethyl)phenyl)propanoic acid

To a stirred solution of methyl 3-(3-(1-(2-(2-fluoro-5-((6-fluoro-4-methyl-1H-indol-5-yl)oxy)phenyl)-1H-imidazol-5-yl)ethyl)phenyl)propanoate (180 mg, 0.35 mmol) in THF (15 mL) was added a solution of lithium hydroxide (51 mg, 2.1 mmol) in water (1 mL). The reaction mixture was stirred at 65° C. for three hours, cooled to room temperature and diluted with water (10 mL). The pH of the resulting mixture was adjusted to ˜4 with 1N hydrochloric acid and extracted with ethyl acetate (50 mL×3). The combined organic extracts were washed with water (10 mL) and brine (10 mL), dried over sodium sulfate, filtered and concentrated. The resulting residue was purified by preparative HPLC to afford the title compound as a white solid (35 mg, 21%). MS: 502 m/z [M+H]⁺. ¹H NMR (400 MHz, CD₃OD) δ 7.39 (dd, J=3.2, 6.0 Hz, 1H), 7.27 (d, J=3.2 Hz, 1H), 7.06-7.22 (m, 6H), 6.84-6.87 (m, 2H), 6.51 (d, J=2.8 Hz, 1H), 4.11 (q, J=7.2 Hz, 1H), 2.89 (t, J=7.6 Hz, 2H), 2.56 (t, J=7.6 Hz, 2H), 2.38 (s, 3H), 1.60 (d, J=7.2 Hz, 3H) ppm.

### Example 214

### 3-(3-(1-(2-(2-Fluoro-5-((6-fluoro-4-methyl-1H-indol-5-yl)oxy)phenyl)-1H-imidazol-5-yl)vinyl)phenyl)propanoic acid

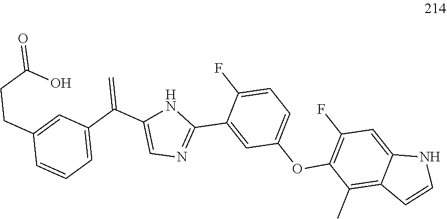 [SMILES: C=C(c1cccc(CCC(=O)O)c1)c1cnc(-c2cc(Oc3c(F)cc4[nH]ccc4c3C)ccc2F)[nH]1]

To a stirred solution of methyl 3-(3-(1-(2-(2-fluoro-5-((6-fluoro-4-methyl-1H-indol-5-yl)oxy)phenyl)-1H-imidazol-5-yl)vinyl)phenyl)propanoate (90 mg, 0.18 mmol, Step B product of Example 213) in THF (10 mL) was added a solution of lithium hydroxide (26 mg, 1.05 mmol) in water (1 mL). The resulting reaction mixture was stirred at 65° C. for three hours, cooled to room temperature and diluted with water (10 mL). The pH of the resulting mixture was adjusted to ˜4 with 1N hydrochloric acid and extracted with ethyl acetate (50 mL×3). The combined organic extracts were washed with water (10 mL) and brine (10 mL), dried over sodium sulfate, filtered and concentrated. The resulting residue was purified by preparative HPLC to afford the title compound as a white solid (30 mg, 34%). MS: 500 m/z [M+H]⁺. ¹H NMR (400 MHz, CD₃OD) δ 7.46 (dd, J=3.2, 6.0 Hz, 1H), 7.33 (s, 1H), 7.10-7.32 (m, 6H), 6.91 (s, 1H), 6.88 (dt, J=8.8, 3.6 Hz, 1H), 6.53 (d, J=3.2 Hz, 1H), 5.72 (s, 1H), 5.26 (s, 1H), 2.84 (t, J=7.6 Hz, 2H), 2.61 (t, J=7.6 Hz, 2H), 2.41 (s, 3H) ppm

### Example 215

### Enantiomer 1 of 3-(3-(1-(2-(2-Fluoro-5-((6-fluoro-4-methyl-1H-indol-5-yl)oxy)phenyl)-1H-imidazol-5-yl)-1-hydroxyethyl)phenyl)propanoic acid (215A)

### Enantiomer 2 of 3-(3-(1-(2-(2-fluoro-5-((6-fluoro-4-methyl-1H-indol-5-yl)oxy)phenyl)-1H-imidazol-5-yl)-1-hydroxyethyl)phenyl)propanoic acid (215B)

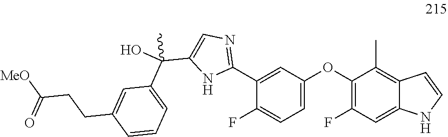 [SMILES: COC(=O)CCc1cccc(C(C)(O)c2cnc(-c3cc(Oc4c(F)cc5[nH]ccc5c4C)ccc3F)[nH]2)c1]

### A. Chiral Separation

Racemic Methyl 3-(3-(1-(2-(2-fluoro-5-((6-fluoro-4-methyl-1H-indol-5-yl)oxy)phenyl)-1H-imidazol-5-yl)-1-hydroxyethyl)phenyl)propanoate (780 mg, product of Step A, Example 213) was separated by Chiral HPLC (Instrument: Gilson-281; Column: Chiralpak AY-H (20×250 mm 10 micron); Column temperature: 35° C.; Mobile phase: n-hexane (modifier: 0.1% diethylamine)/ethanol (modifier: 0.1% diethylamine), v/v, 4/1; Flow rate: 50 mL/min; Detection wavelength: 214 nm, 254 nm) to enantiomer 1 of methyl 3-(3-(1-(2-(2-fluoro-5-((6-fluoro-4-methyl-1H-indol-5-yl)oxy)phenyl)-1H-imidazol-5-yl)-1-hydroxyethyl)phenyl)propanoate (210 mg, Peak 1, retention time 5.6 minutes, SFC) and enantiomer 2 of 3-(3-(1-(2-(2-fluoro-5-((6-fluoro-4-methyl-1H-indol-5-yl)oxy)phenyl)-1H-imidazol-5-yl)-1-hydroxyethyl)phenyl)propanoate (200 mg, Peak 2, retention time 8.2 minutes, SFC), both as white solids.

Example 215A. Enantiomer 1 of 3-(3-(1-(2-(2-Fluoro-5-((6-fluoro-4-methyl-1H-indol-5-yl)oxy)phenyl)-1H-imidazol-5-yl)-1-hydroxyethyl)phenyl)propanoic acid.

To a stirred solution of enantiomer 1 of methyl 3-(3-(1-(2-(2-fluoro-5-((6-fluoro-4-methyl-1H-indol-5-yl)oxy)phenyl)-1H-imidazol-5-yl)-1-hydroxyethyl)phenyl)propanoate (210 mg, 0.4 mmol) in THF (5 mL) was added a solution of lithium hydroxide (29 mg, 1.19 mmol) in water (1 mL) and the resulting mixture was stirred at room temperature overnight. The mixture was diluted with water (10 mL) and THF was removed by evaporation. The aqueous phase was acidified with 1 N hydrochloric acid and the precipitate was collected by filtration to give enantiomer 1 of 3-(3-(1-(2-(2-fluoro-5-((6-fluoro-4-methyl-1H-indol-5-yl)oxy)phenyl)-1H-imidazol-5-yl)-1-hydroxyethyl)phenyl)propanoic acid as a white solid (140 mg, 68%). MS: 518 m/z [M+H]⁺. ¹H NMR (400 MHz, CD₃OD) δ 7.40 (dd, J=3.2, 6.0 Hz, 1H), 7.37 (s, 1H), 7.29-7.27 (m, 2H), 7.23 (t, J=7.2 Hz, 1H), 7.18-7.09 (m, 3H), 6.94 (s, 1H), 6.88-6.85 (dt, J=3.3, 8.8 Hz, 1H), 6.52 (d, J=3.2 Hz, 1H), 2.91 (t, J=7.6 Hz, 2H), 2.57 (t, J=8.0 Hz, 2H), 2.39 (s, 3H), 1.87 (s, 3H) ppm. LC-MS Retention time: 0.87 minutes

### Example 215B. Enantiomer 2 of 3-(3-(1-(2-(2-fluoro-5-((6-fluoro-4-methyl-1H-indol-5-yl)oxy)phenyl)-1H-imidazol-5-yl)-1-hydroxyethyl)phenyl)propanoic acid

Following the same procedure as described for Example 215A, enantiomer 2 of methyl 3-(3-(1-(2-(2-fluoro-5-((6-fluoro-4-methyl-1H-indol-5-yl)oxy)phenyl)-1H-imidazol-5-yl)-1-hydroxyethyl)phenyl)propanoate (200 mg) was hydrolyzed to enantiomer 2 of 3-(3-(1-(2-(2-fluoro-5-((6-fluoro-4-methyl-1H-indol-5-yl)oxy)phenyl)-1H-imidazol-5-yl)-1-hydroxyethyl)phenyl)propanoic acid as a white solid (100 mg, 51%). MS: 518 m/z [M+H]⁺. ¹H NMR (400 MHz, CD₃OD) δ δ 7.40 (dd, J=3.2, 6.0 Hz, 1H), 7.37 (s, 1H), 7.28-7.27 (m, 2H), 7.23 (t, J=6.0 Hz, 1H), 7.18-7.09 (m, 3H), 6.94 (s, 1H), 6.88-6.85 (m, 1H), 6.52 (t, J=3.2 Hz, 1H), 2.91 (t, J=6.4 Hz, 2H), 2.57 (t, J=6.4 Hz, 2H), 2.39 (s, 3H), 1.87 (s, 3H) ppm. LC-MS Retention time: 0.87 minutes.

The following examples were prepared utilizing the similar procedures described for Example 215A/215B.

| | Ex. | | | LCMS | MS m/z |
|---|---|---|---|---|---|
| Structure | No. | Name | ¹H NMR | Rₜ (min) | [M + H]⁺ |
| 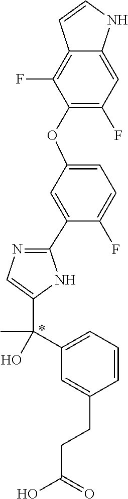 [SMILES: CC(O)(c1cccc(CCC(=O)O)c1)c1cnc(-c2cc(Oc3c(F)cc4[nH]ccc4c3F)ccc2F)[nH]1] | 216A | Enantiomer 1 of 3-(3- (1-(2-(5-((4,6- difluoro-1H-indol-5- yl)oxy)-2- fluorophenyl)-1H- imidazol-5-yl)-1- hydroxyethyl)phenyl) propanoic acid | (400 MHz, CD₃OD) δ 7.50 (dd, J = 3.2, 6.0 Hz, 1H), 7.38 (s, 1H), 7.31 (d, J = 3.2 Hz, 1H), 7.29-7.25 (m, 1H), 7.23-7.17 (m, 2H), 7.15-7.11 (m, 2H), 6.98 (dt, J = 8.8, 3.6 Hz, 1H), 6.95 (s, 1H), 6.56 (d, J = 2.8 Hz, 1H), 2.91 (t, J = 7.2 Hz, 2H), 2.58 (t, J = 7.6 Hz, 2H), 1.87 (s, 3H) ppm. | 0.88 | 522 |
| 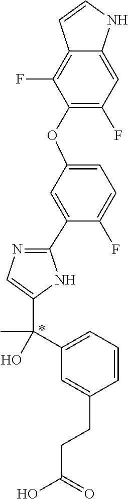 [SMILES: CC(O)(c1cccc(CCC(=O)O)c1)c1cnc(-c2cc(Oc3c(F)cc4[nH]ccc4c3F)ccc2F)[nH]1] | 216B | Enantiomer 2 of 3-(3- (1-(2-(5-((4,6- difluoro-1H-indol-5- yl)oxy)-2- fluorophenyl)-1H- imidazol-5-yl)-1- hydroxyethyl)phenyl) propanoic acid | (400 MHz, CD₃OD) δ 7.50 (dd, J = 3.2, 6.0 Hz, 1H), 7.38 (s, 1H), 7.31 (d, J = 3.2 Hz, 1H), 7.29-7.25 (m, 1H), 7.23-7.17 (m, 2H), 7.15-7.11 (m, 2H), 6.98 (td, J = 3.6, 8.8 Hz, 1H), 6.95 (s, 1H), 6.56 (d, J = 2.8 Hz, 1H), 2.91 (t, J = 7.6 Hz, 2H), 2.58 (t, J = 7.6 Hz, 2H), 1.88 (s, 3H) ppm. | 0.87 | 522 |

a. Chiral separation conditions: instrument: Gilson-281; Column: 30 × 250 mm × 5 μM Phenomenex Cellulose-3; mobile phase: 80:20 hexane/ethanol with 0.1% diethylamine modifier; flow rate: 50 mL/min.

### Example 217

### 3-(3-((2-(2-Fluoro-5-((6-fluoro-4-methyl-1H-indol-5-yl)oxy)phenyl)-4-methyl-1H-imidazol-5-yl)(hydroxy)methyl)phenyl)propanoic acid

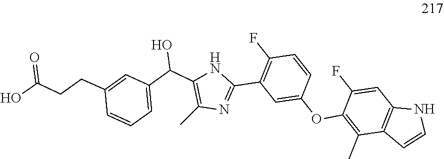 [SMILES: Cc1nc(-c2cc(Oc3c(F)cc4[nH]ccc4c3C)ccc2F)[nH]c1C(O)c1cccc(CCC(=O)O)c1]

### A. Ethyl 3-(3-formylphenyl)propanoate

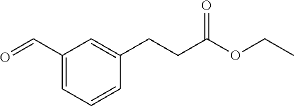

A mixture of 3-bromobenzaldehyde (2 g, 10.81 mmol), 3,3-diethoxyprop-1-ene (4.22 g, 32.43 mmol), tetrabutylammonium chloride (3.0 g, 10.81 mmol), tributylamine (4 g, 21.62 mmol) and palladium(II) acetate (224 mg, 1 mmol) in DMF (10 mL) was stirred at 90° C. for three hours in a glove box. The reaction mixture was filtered through a pad of Celite and the filter cake was washed with ethyl acetate (50 mL×3). The combined filtrates were washed with water (20 mL×2) and brine (20 mL×3), dried over sodium sulfate, filtered and concentrated. The resulting residue was purified by flash chromatography (10% ethyl acetate in petroleum ether) to afford the title compound as a yellow oil (1.3 g, 58%). MS: 207 m/z [M+H]⁺.

### B. (E)-3-(3-(3-Oxobut-1-en-1-yl)phenyl)propanoic acid

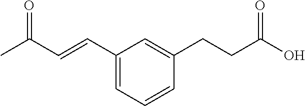 [SMILES: CC(=O)/C=C/c1cccc(CCC(=O)O)c1]

To a mixture of solid sodium hydroxide (252 mg, 6.31 mmol) and acetone (1.46 g, 25.24 mmol) in water (3 mL) and ethanol (1.5 mL) was added ethyl 3-(3-formylphenyl)propanoate (1.3 g, 6.31 mmol) dropwise over ten minutes at 0° C. The reaction mixture was stirred at room temperature for thirty minutes, acidified to pH˜4 with aqueous hydrochloric acid (1 N) and then extracted with ethyl acetate (3×50 mL). The combined organic extracts were washed with brine, dried over sodium sulfate and concentrated to afford the title compound as yellow oil (1.4 g, crude), which was used for the next step without further purification. MS: 219 m/z [M+H]⁺.

### C. Methyl (E)-3-(3-(3-oxobut-1-en-1-yl)phenyl)propanoate

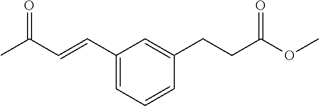 [SMILES: COC(=O)CCc1cccc(/C=C/C(C)=O)c1]

To a stirred solution of (E)-3-(3-(3-oxobut-1-en-1-yl)phenyl)propanoic acid (1.4 g, 6.42 mmol) in methanol (15 mL) was added concentrated sulfuric acid (0.5 mL). The reaction mixture was stirred at 65° C. for 2.5 hours and concentrated. The resulting residue was poured into ice-water (40 mL) and extracted with ethyl acetate (30 mL×2). The combined organic phase was washed with brine (10 mL×2), dried over sodium sulfate, filtered and concentrated. The resulting residue was purified by flash chromatography (10% ethyl acetate in petroleum ether) to afford the title compound as a colorless oil (620 mg, 49% from two steps). MS: 233 m/z [M+H]⁺.

### D. Methyl 3-(3-(3-acetyloxiran-2-yl)phenyl)propanoate

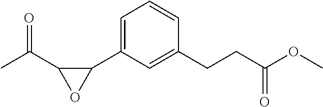 [SMILES: COC(=O)CCc1cccc(C2OC2C(C)=O)c1]

To a stirred solution of methyl (E)-3-(3-(3-oxobut-1-en-1-yl)phenyl)propanoate (600 mg, 2.59 mmol) in methanol (10 mL) was added hydrogen peroxide (440 mg, 3.88 mmol, 30% in water) while maintaining the pH at 10.0-10.5 with 1N sodium hydroxide. The reaction mixture was stirred at room temperature for two hours, diluted with ether (100 mL), washed with water (15 mL×2) and brine (10 mL), dried over sodium sulfate and concentrated. The resulting residue was purified by flash chromatography (10% ethyl acetate in petroleum ether) to afford the title compound as a colorless oil (320 mg, 50%). MS: 249 m/z [M+H]⁺.

### E. Example 217. 3-(3-((2-(2-Fluoro-5-((6-fluoro-4-methyl-1H-indol-5-yl)oxy)phenyl)-4-methyl-1H-imidazol-5-yl)(hydroxy)methyl)phenyl)propanoic acid

The title compound was prepared in two steps as described in Example 172, steps J to K but exchanging methyl 2-fluoro-3-(3-(3-formyloxiran-2-yl)phenyl)propanoate for methyl 3-(3-(3-acetyloxiran-2-yl)phenyl)propanoate and 3-((4-methyl-1H-indol-5-yl)oxy)benzimidamide for 2-fluoro-5-((6-fluoro-4-methyl-1H-indol-5-yl)oxy)benzimidamide. LC-MS retention time: 0.92 minutes. MS: 518 m/z [M+H]⁺. ¹H NMR (400 MHz, CD₃OD) δ 7.37 (dd, J=6.4, 3.2 Hz, 1H), 7.32 (s, 1H), 7.27 (d, J=2.8 Hz, 1H), 7.26-7.24 (m, 2H), 7.18-7.09 (m, 3H), 6.87 (dt, J=8.8, 3.6 Hz, 1H), 6.52 (dd, J=3.2, 0.8 Hz, 1H), 5.87 (s, 1H), 2.91 (t, J=7.6 Hz, 2H), 2.57 (t, J=7.6 Hz, 2H), 2.40 (s, 3H), 2.10 (s, 3H) ppm.

### Example 218

### 3-(3-(1-(2-(2-Fluoro-5-(hydroxy(1H-indol-5-yl)methyl)phenyl)-1H-imidazol-4-yl)-1-hydroxyethyl)phenyl)propanoic acid

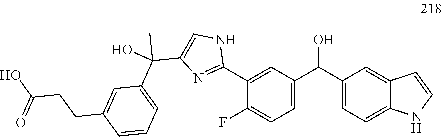 [SMILES: CC(O)(c1cccc(CCC(=O)O)c1)c1c[nH]c(-c2cc(C(O)c3ccc4[nH]ccc4c3)ccc2F)n1]

To a stirred solution of 3-(3-(1-(2-(2-fluoro-5-(1H-indole-5-carbonyl)phenyl)-1H-imidazol-4-yl)-1-hydroxyethyl)phenyl)propanoic acid (50 mg, 1.0 mmol, Example 194) in methanol (5 mL) was added sodium borohydride (114 mg, 3.0 mmol) at 0° C. The reaction mixture was stirred at 0° C. for four hours, acidified to pH 3-4 with 1N hydrochloric acid and extracted with ethyl acetate (25 mL×3). The combined organic extracts were washed with brine, dried over sodium sulfate and concentrated. The resulting residue was purified with preparative HPLC to afford the title compound as a white solid (11.2 mg, 23%). MS: 500 m/z [M+H]⁺. ¹H NMR (400 MHz, CD₃OD) δ 8.00 (dd, J=7.2, 2.4 Hz, 1H), 7.59 (s, 1H), 7.49-7.43 (m, 1H), 7.41 (s, 1H), 7.35-7.17 (m, 5H), 7.16-7.11 (m, 2H), 6.98 (d, J=2.4 Hz, 1H), 6.42 (d, J=3.2 Hz, 1H), 5.92 (s, 1H), 2.92 (t, J=7.6 Hz, 2H), 2.58 (t, J=7.6 Hz, 2H), 1.91 (s, 3H) ppm.

### Example 219

### (2R)-1-(5-(1-(2-(3-((4,6-Difluoro-1H-indol-5-yl)oxy)phenyl)-1H-imidazol-5-yl)-1-hydroxyethyl)isoindolin-2-yl)-2-hydroxypropan-1-one

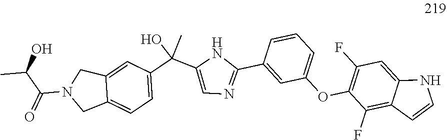 [SMILES: C[C@@H](O)C(=O)N1Cc2ccc(C(C)(O)c3cnc(-c4cccc(Oc5c(F)cc6[nH]ccc6c5F)c4)[nH]3)cc2C1]

### A. (2R)-2-(Benzyloxy)-1-(5-(1-(2-(3-((4,6-difluoro-1H-indol-5-yl)oxy)phenyl)-1H-imidazol-5-yl)-1-hydroxyethyl)isoindolin-2-yl)propan-1-one

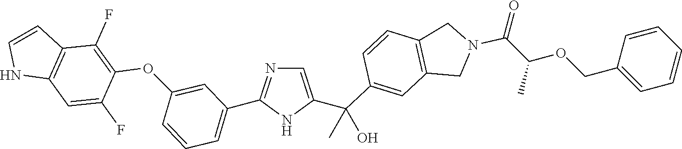 [SMILES: C[C@@H](OCc1ccccc1)C(=O)N1Cc2ccc(C(C)(O)c3cnc(-c4cccc(Oc5c(F)cc6[nH]ccc6c5F)c4)[nH]3)cc2C1]

Exchanging methyl 2-fluoro-3-(3-(3-formyloxiran-2-yl)phenyl)propanoate for 3-(2-((R)-2-(benzyloxy)propanoyl)isoindolin-5-yl)-3-methyloxirane-2-carbaldehyde (Intermediate 26-6, 353 mg, 1.23 mmol) and 3-((4-methyl-1H-indol-5-yl)oxy)benzimidamide for 3-((4,6-difluoro-1H-indol-5-yl)oxy)benzimidamide (Intermediate 24-2, 540 mg, 1.48 mmol), the reaction conditions described for Step J of Example 173 was used to prepare the title compound as a yellow solid (80 mg, 10.2%). MS: 635[M+H]⁺.

### B. Example 219. (2R)-1-(5-(1-(2-(3-((4,6-Difluoro-1H-indol-5-yl)oxy)phenyl)-1H-imidazol-5-yl)-1-hydroxyethyl)isoindolin-2-yl)-2-hydroxypropan-1-one

To a stirred solution of (2R)-2-(benzyloxy)-1-(5-(1-(2-(3-((4,6-difluoro-1H-indol-5-yl)oxy)phenyl)-1H-imidazol-5-yl)-1-hydroxyethyl)isoindolin-2-yl)propan-1-one (92 mg, 0.145 mmol) in methanol (20 mL) was added Pd/C (10%, 276 mg). The reaction mixture was stirred at room temperature under hydrogen atmosphere for eighteen hours and filtered through a pad of Celite. The filter cake was washed with methanol (30 mL×6) and DMF (3 mL). The filtrate was concentrated and the residue was purified by preparative HPLC to afford the title compound as a light yellow solid (4 mg, 5%). LC-MS retention time: 0.76 min. MS: 545 m/z [M+H]⁺.

### Example 220

### 3-(3-(1-(2-(5-((4,6-Difluoro-1H-indol-5-yl)oxy)-2-fluorophenyl)-1H-imidazol-4-yl)-1-hydroxyethyl)-5-fluorophenyl)propanoic acid

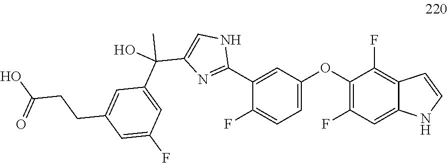 [SMILES: CC(O)(c1cc(F)cc(CCC(=O)O)c1)c1c[nH]c(-c2cc(Oc3c(F)cc4[nH]ccc4c3F)ccc2F)n1]

### And

### Example 221

### 4-(3-(1-(2-(5-((4,6-Difluoro-1H-indol-5-yl)oxy)-2-fluorophenyl)-1H-imidazol-4-yl)-1-hydroxyethyl)-5-fluorophenyl)butan-2-one

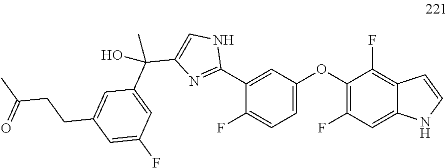 [SMILES: CC(=O)CCc1cc(F)cc(C(C)(O)c2c[nH]c(-c3cc(Oc4c(F)cc5[nH]ccc5c4F)ccc3F)n2)c1]

### A. Methyl 3-(3-((2-(5-((4,6-difluoro-1H-indol-5-yl)oxy)-2-fluorophenyl)-1H-imidazol-4-yl)(hydroxy)methyl)-5-fluorophenyl)propanoate

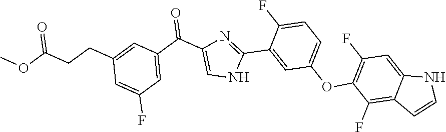 [SMILES: COC(=O)CCc1cc(F)cc(C(=O)c2c[nH]c(-c3cc(Oc4c(F)cc5[nH]ccc5c4F)ccc3F)n2)c1]

Exchanging methyl 2-fluoro-3-(3-(3-formyloxiran-2-yl)phenyl)propanoate for methyl 3-(3-fluoro-5-(3-formyloxiran-2-yl)phenyl)propanoate (463 mg, 1.98 mmol, Intermediate 26-5) and 3-((4-methyl-1H-indol-5-yl)oxy)benzimidamide for 5-((4,6-difluoro-1H-indol-5-yl)oxy)-2-fluorobenzimidamide (604 mg, 1.98 mmol, Intermediate 24-9), the reaction conditions described in Step J of Example 172 was used to prepare the title compound as a light brown solid (420 mg, 40%). MS: 540 m/z [M+H]⁺.

### B. Methyl 3-(3-(2-(5-((4,6-difluoro-1H-indol-5-yl)oxy)-2-fluorophenyl)-1H-imidazole-4-carbonyl)-5-fluorophenyl)propanoate

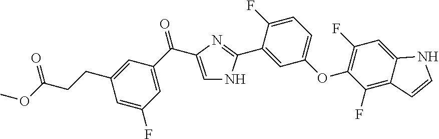 [SMILES: COC(=O)CCc1cc(F)cc(C(=O)c2c[nH]c(-c3cc(Oc4c(F)cc5[nH]ccc5c4F)ccc3F)n2)c1]

To a stirred solution of methyl 3-(3-((2-(5-((4,6-difluoro-1H-indol-5-yl)oxy)-2-fluorophenyl)-1H-imidazol-4-yl)(hydroxy)methyl)-5-fluorophenyl)propanoate (1.2 g, 2.23 mmol) in THF (25 mL) was added manganese dioxide (1.98 g, 22.26 mmol). The resulting mixture was stirred overnight at 25° C. and filtered through a pad of Celite. The filter cake was washed with THF (10 mL×4) and the filtrate was concentrated to afford the title compound as a yellow solid (1.15 g, 96%), which was used directly for the next step. MS: 538 m/z [M+H]⁺.

### C. 3-(3-(2-(5-((4,6-Difluoro-1H-indol-5-yl)oxy)-2-fluorophenyl)-1H-imidazole-4-carbonyl)-5-fluorophenyl)propanoic acid

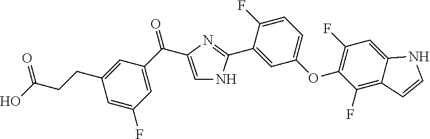 [SMILES: O=C(O)CCc1cc(F)cc(C(=O)c2c[nH]c(-c3cc(Oc4c(F)cc5[nH]ccc5c4F)ccc3F)n2)c1]

To a stirred solution of methyl 3-(3-(2-(5-((4,6-difluoro-1H-indol-5-yl)oxy)-2-fluorophenyl)-1H-imidazole-4-carbonyl)-5-fluorophenyl)propanoate (500 mg, 0.96 mmol) in THF (10 mL) was added a solution of lithium hydroxide (160 mg, 6.69 mmol) in water (2 mL). The reaction mixture was stirred at room temperature overnight and concentrated to remove THF. The resulting residue was dissolved in 5 mL of water and acidified to pH˜4 with 1 M HCl. The white solid was collected by filtration and dried to afford the title compound as a light yellow solid (480 mg, 98%). MS: 524 m/z [M+H]⁺.

### D. Example 220. 3-(3-(1-(2-(5-((4,6-Difluoro-1H-indol-5-yl)oxy)-2-fluorophenyl)-1H-imidazol-4-yl)-1-hydroxyethyl)-5-fluorophenyl)propanoic acid and Example 221. 4-(3-(1-(2-(5-((4,6-Difluoro-1H-indol-5-yl)oxy)-2-fluorophenyl)-1H-imidazol-4-yl)-1-hydroxyethyl)-5-fluorophenyl)butan-2-one

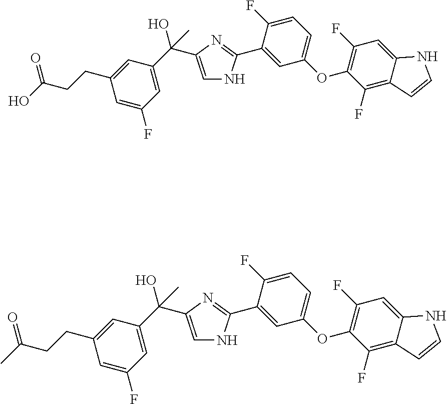 [SMILES: CC(O)(c1cc(F)cc(CCC(=O)O)c1)c1c[nH]c(-c2cc(Oc3c(F)cc4[nH]ccc4c3F)ccc2F)n1 ; CC(=O)CCc1cc(F)cc(C(C)(O)c2c[nH]c(-c3cc(Oc4c(F)cc5[nH]ccc5c4F)ccc3F)n2)c1]

To a stirred solution of 3-(3-(2-(5-((4,6-difluoro-1H-indol-5-yl)oxy)-2-fluorophenyl)-1H-imidazole-4-carbonyl)-5-fluorophenyl)propanoic acid (210 mg, 0.41 mmol) in dioxane (45 mL) was added methyllithium (1.92 mL, 5.74 mmol, 3 N in diethyl ether) at 0° C. The reaction mixture was stirred at room temperature for three hours, quenched with water (20 mL) and concentrated. The residue was diluted with ethyl acetate (80 mL), washed with water (20 mL×2) and brine (15 mL×2), dried over sodium sulfate, filtered and concentrated. The resulting residue was purified by preparative HPLC to afford Example 220 and Example 221.

Example 220 obtained as a white solid (60 mg, 28%), MS: 540 m/z [M+H]⁺. ¹H NMR (400 MHz, CD₃OD) δ 7.51 (dd, J=3.2, 6.0 Hz, 1H), 7.30 (d, J=3.2 Hz, 1H), 7.22-7.12 (m, 3H), 7.06 (d, J=10.4 Hz, 1H), 6.99-6.96 (m, 2H), 6.86 (d, J=9.6 Hz, 1H), 6.56 (d, J=2.8 Hz, 1H), 2.90 (t, J=7.6 Hz, 2H), 2.57 (t, J==7.6 Hz, 2H), 1.86 (s, 3H) ppm.

Example 221 obtained as a white solid (30 mg, 14%). MS: 538 m/z [M+H]⁺. ¹H NMR (400 MHz, CD₃OD) δ 7.52 (m, 1H), 7.31 (d, J=3.2 Hz, 1H), 7.21-7.04 (m, 4H), 6.98 (m, 2H), 6.84 (d, J=10 Hz, 1H), 6.56 (d, J=3.2 Hz, 1H), 2.83-2.77 (m, 4H), 2.10 (s, 3H), 1.86 (s, 3H) ppm.

The following examples were prepared utilizing similar procedures as described for Example 221.

| | Ex. | | | LCMS | MS |
|---|---|---|---|---|---|
| Structure | No. | Name | 1H NMR | Rₜ (min) | (m/z) |
| 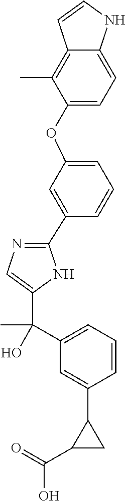 [SMILES: Cc1c(Oc2cccc(-c3ncc(C(C)(O)c4cccc(C5CC5C(=O)O)c4)[nH]3)c2)ccc2[nH]ccc12] | 222 | 2-(3-(1-hydroxy-1-(2-(3-((4- methyl-1H-indol-5- yl)oxy)phenyl)-1H-imidazol- 5- yl)ethyl)phenyl)cyclopropane- 1-carboxylic acid | (400 MHz, CD₃OD) δ 7.47 (d, J = 7.2 Hz, 1H), 7.37 (s, 1H), 7.35- 7.23 (m, 6H), 7.01 (d, J = 6.4 Hz, 1H), 6.91 (s, 1H), 6.82 (dd, J = 3.2, 5.2 Hz, 2H), 6.52 (d, J = 2.8 Hz, 1H), 2.45-2.37 (m, 1H), 2.35 (s, 3H), 1.88 (s, 3H), 1.88-1.83 (m, 1H), 1.51-1.49 (m, 1H), 1.30-1.28 (m, 1H) ppm. | 0.87 | 494 [M + H]⁺ |
| 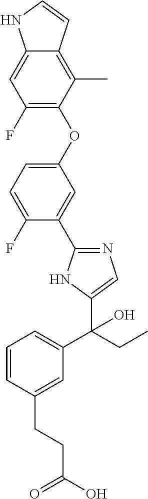 [SMILES: CCC(O)(c1cccc(CCC(=O)O)c1)c1cnc(-c2cc(Oc3c(F)cc4[nH]ccc4c3C)ccc2F)[nH]1] | 223 | 3-(3-(1-(2-(2-fluoro-5-((6- fluoro-4-methyl-1H-indol-5- yl)oxy)phenyl)-1H-imidazol- 5-yl)-1- hydroxypropyl)phenyl) propanoic acid | (400 MHz, CD₃OD) δ 7.40 (dd, J = 3.2. 6.0 Hz, 1H), 7.36 (s, 1H), 7.28-7.27 (m, 2H), 7.22 (t, J = 7.6 Hz, 1H), 7.17-7.09 (m, 3H), 6.98 (s, 1H), 6.86 (dt, J = 8.8, 3.6 Hz, 1H), 6.52 (d, J = 3.2 Hz, 1H), 2.90 (t, J = 7.6 Hz, 2H), 2.56 (t, J = 7.6 Hz, 2H), 2.39 (s, 3H), 2.32-2.16 (m, 2H), 0.84 (d, J = 7.2 Hz, 3H) ppm. | 0.94 | 532 [M + H]⁺ |
| 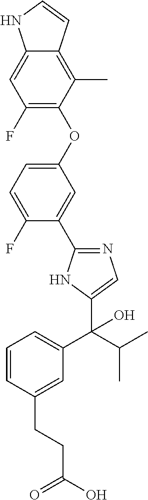 [SMILES: Cc1c(Oc2ccc(F)c(-c3ncc(C(O)(c4cccc(CCC(=O)O)c4)C(C)C)[nH]3)c2)c(F)cc2[nH]ccc12] | 224 | 3-(3-(1-(2-(2-fluoro-5-((6- fluoro-4-methyl-1H-indol-5- yl)oxy)phenyl)-1H-imidazol- 5-yl)-1-hydroxy-2- methylpropyl)phenyl) propanoic acid | (400 MHz, CD₃OD) δ 7.44-7.38 (m, 2H), 7.35 (d, J = 8.0 Hz, 1H), 7.26 (d, J = 3.2 Hz, 1H), 7.19-7.03 (m, 5H), 6.84-6.78 (m, 1H), 6.50 (d, J = 2.8 Hz, 1H), 2.89 (t, J = 7.6 Hz, 2H), 2.69-2.61 (m, 1H), 2.56 (t, J = 7.6 Hz, 2H), 2.39 (s, 3H), 0.96 (d, J = 6.8 Hz, 3H), 0.77 (d, J = 6.8 Hz, 3H) ppm. | 1.01 | 546 [M + H]⁺ |
| 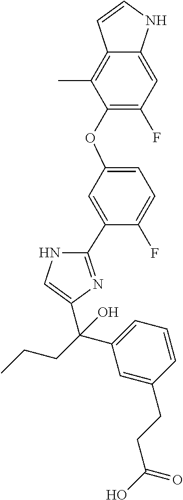 [SMILES: CCCC(O)(c1cccc(CCC(=O)O)c1)c1c[nH]c(-c2cc(Oc3c(F)cc4[nH]ccc4c3C)ccc2F)n1] | 225 | 3-(3-(1-(2-(2-fluoro-5-((6- fluoro-4-methyl-1H-indol-5- yl)oxy)phenyl)-1H-imidazol- 4-yl)-1- hydroxybutyl)phenyl) propanoic acid | (400 MHz, CD₃OD) δ 7.40 (dd, J = 5.6, 2.8 Hz, 1H), 7.36 (s, 1H), 7.29-7.20 (m, 3H), 7.18- 7.09 (m, 3H), 6.98 (s, 1H), 6.87-6.85 (m, 1H), 6.52 (d, J = 2.0 Hz, 1H), 2.91 (t, J = 7.6 Hz, 2H), 2.57 (t, J = 7.6 Hz, 2H), 2.39 (s, 3H), 2.25-2.21 (m, 1H), 2.17-2.13 (m, 1H), 1.39-1.36 (m, 1H), 1.22-1.20 (m, 1H), 0.90 (t, J = 7.2 Hz, 3H) ppm. | 1.05 | 546 [M + H]⁺ |
| 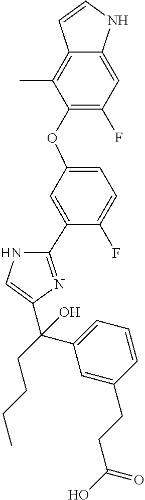 [SMILES: CCCCC(O)(c1cccc(CCC(=O)O)c1)c1c[nH]c(-c2cc(Oc3c(F)cc4[nH]ccc4c3C)ccc2F)n1] | 226 | 3-(3-(1-(2-(2-fluoro-5-((6- fluoro-4-methyl-1H-indol-5- yl)oxy)phenyl)-1H-imidazol- 4-yl)-1- hydroxypentyl)phenyl) propanoic acid | (500 MHz, CD₃OD) δ 7.40 (dd, J = 4.8, 2.8 Hz, 1H), 7.37 (s, 1H), 7.29-7.27 (m, 2H), 7.22 (dd, J = 6.0, 6.0 Hz, 1H), 7.17- 7.10 (m, 3H), 6.97 (s, 1H), 6.88-6.86 (dt, J = 7.2, 2.8 Hz, 1H), 6.52 (d, J = 8.0 Hz, 1H), 2.91 (t, J = 8.0 Hz, 2H), 2.57 (t, J = 8.0 Hz, 2H), 2.39 (s, 3H), 2.27-2.24 (m, 1H), 2.18-2.16 (m, 1H), 1.35-1.28 (m, 3H), 1.17-1.14 (m, 1H), 0.87 (t, J = 7.0 Hz, 3H) ppm. | 1.11 | 560 [M+H]+ |

### Example 227

### 3-(3-(2-(2-(2-Fluoro-5-((4,6,7-trifluoro-1H-indol-5-yl)oxy)phenyl)-1H-imidazol-5-yl)-5,5-dimethyl-1,3-dioxan-2-yl)phenyl)propanoic acid

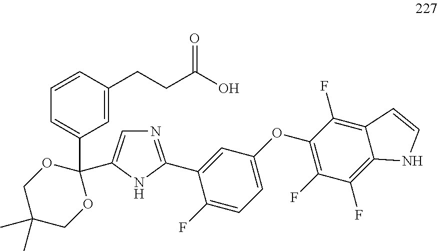 [SMILES: CC1(C)COC(c2cccc(CCC(=O)O)c2)(c2cnc(-c3cc(Oc4c(F)c(F)c5[nH]ccc5c4F)ccc3F)[nH]2)OC1]

### A. Ethyl 3-(3-((2-(2-fluoro-5-((4,6,7-trifluoro-1H-indol-5-yl)oxy)phenyl)-1H-imidazol-5-yl)(hydroxy)methyl)phenyl)propanoate

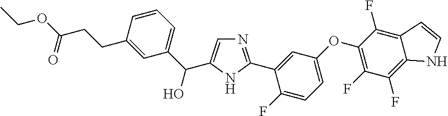 [SMILES: CCOC(=O)CCc1cccc(C(O)c2cnc(-c3cc(Oc4c(F)c(F)c5[nH]ccc5c4F)ccc3F)[nH]2)c1]

To a stirred solution of 2-fluoro-5-((4,6,7-trifluoro-1H-indol-5-yl)oxy)benzimidamide (Intermediate 24-17, 500 mg, 1.55 mmol) in DMF (10 mL) was added ethyl 3-(3-(3-formyloxiran-2-yl)phenyl)propanoate (Intermediate 26-4, 390 mg, 1.55 mmol). The reaction mixture was stirred overnight at 75° C., cooled to room temperature and diluted with ethyl acetate (60 mL) and water (30 mL). The organic phase was washed with brine, dried over sodium sulfate and concentrated. The resulting residue was purified by flash chromatography over silica (petroleum ether/dichloromethane/ethyl acetate, v/v, 2/1/1) to afford the title compound as a white solid (410 mg, 48%). MS: 554 m/z [M+H]⁺.

### B. Ethyl 3-(3-(2-(2-fluoro-5-((4,6,7-trifluoro-1H-indol-5-yl)oxy)phenyl)-1H-imidazole-5-carbonyl)phenyl)propanoate

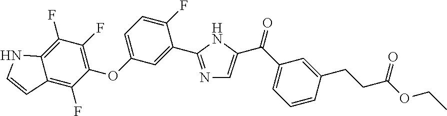 [SMILES: CCOC(=O)CCc1cccc(C(=O)c2cnc(-c3cc(Oc4c(F)c(F)c5[nH]ccc5c4F)ccc3F)[nH]2)c1]

To a stirred solution of ethyl 3-(3-((2-(2-fluoro-5-((4,6,7-trifluoro-1H-indol-5-yl)oxy)phenyl)-1H-imidazol-5-yl)(hydroxy)methyl)phenyl)propanoate (490 mg, 0.886 mmol) in THF (10 mL) was added manganese dioxide (775 mg, 8.9 mmol). The reaction mixture was stirred overnight at 25° C., diluted with ethyl acetate (50 mL) and filtered through a pad of Celite. The filtrate was concentrated and the resulting residue was purified by flash chromatography over silica (petroleum ether/dichloromethane/ethyl acetate, v/v, 2/1/1) to afford the title compound as a white solid (400 mg, 82%). MS: 552 m/z [M+H]⁺.

### C. Ethyl 3-(3-(2-(2-(2-fluoro-5-((4,6,7-trifluoro-1H-indol-5-yl)oxy)phenyl)-1H-imidazol-5-yl)-5,5-dimethyl-1,3-dioxan-2-yl)phenyl)propanoate

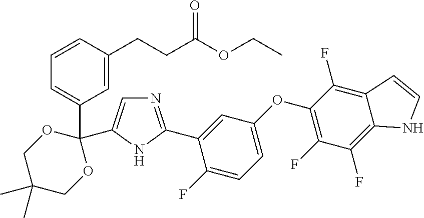 [SMILES: CCOC(=O)CCc1cccc(C2(c3cnc(-c4cc(Oc5c(F)c(F)c6[nH]ccc6c5F)ccc4F)[nH]3)OCC(C)(C)CO2)c1]

To a stirred solution of ethyl 3-(3-(2-(2-fluoro-5-((4,6,7-trifluoro-1H-indol-5-yl)oxy)phenyl)-1H-imidazole-5-carbonyl)phenyl)propanoate (445 mg, 0.807 mmol) in toluene (10 mL) were added 2,2-dimethylpropane-1,3-diol (1.26 g, 12.1 mmol), p-toluenesulfonic acid (70 mg, 0.404 mmol) and anhydrous magnesium sulfate (500 mg). The reaction mixture was heated at 120° C. overnight, cooled to room temperature and diluted with ethyl acetate (100 mL) and water (30 mL). The organic phase was washed with brine, dried and concentrated. The residue was purified by flash chromatography over silica (petroleum ether/dichloromethane/ethyl acetate, v/v, 3/2/2) to afford the title compound as a white solid (94 mg, 20%). MS: 638 m/z [M+H]⁺.

### D. Example 227. 3-(3-(2-(2-(2-Fluoro-5-((4,6,7-trifluoro-1H-indol-5-yl)oxy)phenyl)-1H-imidazol-5-yl)-5,5-dimethyl-1,3-dioxan-2-yl)phenyl)propanoic acid

To a stirred solution of ethyl 3-(3-(2-(2-(2-fluoro-5-((4,6,7-trifluoro-1H-indol-5-yl)oxy)phenyl)-1H-imidazol-5-yl)-5,5-dimethyl-1,3-dioxan-2-yl)phenyl)propanoate (94 mg, 0.15 mmol) in a 5:1 mixture of THF and water (12 mL) was added lithium hydroxide (25 mg, 1.03 mmol). The reaction mixture was stirred at 25° C. overnight and concentrated to remove THF. The residue was acidified to pH˜4 with 1N hydrochloric acid and the precipitate was collected by filtration. The filter cake was purified by preparative HPLC to afford the title compound as a white solid (30 mg, 34%). ¹H NMR (400 MHz, CD₃OD) δ 7.58-7.56 (m, 1H), 7.45 (s, 1H), 7.39-7.36 (m, 2H), 7.27-7.22 (m, 2H), 7.18 (t, J=8.0 Hz, 1H), 7.05-7.01 (m, 2H), 6.64 (t, J=2.0 Hz, 1H), 3.70 (d, J=11.2 Hz, 2H), 3.59 (d, J=11.2 Hz, 2H), 2.90 (t, J=7.6 Hz, 2H), 2.59 (t, J=7.6 Hz, 2H), 0.99 (d, J=11.2 Hz, 6H) ppm. MS: 610 m/z [M+H]⁺.

The following examples were prepared utilizing similar procedures as described for Example 227.

| | Ex. | | | LCMS | MS m/z |
|---|---|---|---|---|---|
| Structure | No. | Name | ¹H NMR | Rₜ (min) | [M + H]⁺ |
| 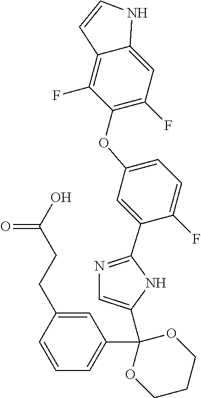 [SMILES: O=C(O)CCc1cccc(C2(c3cnc(-c4cc(Oc5c(F)cc6[nH]ccc6c5F)ccc4F)[nH]3)OCCCO2)c1] | 228 | 3-(3-(2-(2-(5-((4,6- difluoro-1H-indol-5- yl)oxy)-2- fluorophenyl)-1H- imidazol-5-yl)-1,3- dioxan-2- yl)phenyl)propanoic acid | (400 MHz, CD₃OD) δ 7.57 (m, 1H), 7.45 (m, 1H), 7.38 (m, 1H), 7.31- 7.27 (m, 2H), 7.21-7.13 (m, 3H), 6.98 (m, 1H), 6.79 (s, 1H), 6.56 (d, J = 2.8 Hz, 1H), 4.06-3.99 (m, 4H), 2.95 (t, J = 7.8 Hz, 2H), 2.61 (t, J = 7.8 Hz, 2H), 1.93 (m, 1H), 1.71 (m, 1H) ppm | 1.00 | 564 |
| 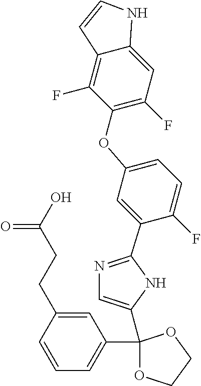 [SMILES: O=C(O)CCc1cccc(C2(c3cnc(-c4cc(Oc5c(F)cc6[nH]ccc6c5F)ccc4F)[nH]3)OCCO2)c1] | 229 | 3-(3-(2-(2-(5-((4,6- difluoro-1H-indol-5- yl)oxy)-2- fluorophenyl)-1H- imidazol-5-yl)-1,3- dioxolan-2- yl)phenyl)propanoic acid | (400 MHz, CD₃OD) δ 7.56 (dd, J = 5.8, 3.3 Hz, 1H), 7.46 (s, 1H), 7.38 (d, J = 7.6 Hz, 1H), 7.32-7.12 (m, 5H), 6.99 (dd, J = 8.1, 4.6 Hz, 1H), 6.80 (s, 1H), 6.56 (d, J = 2.6 Hz, 1H), 4.15 (t, J = 6.8 Hz, 2H), 3.97 (t, J = 6.8 Hz, 2H), 2.94 (t, J = 7.7 Hz, 2H), 2.58 (t, J = 7.7 Hz, 2H) ppm. | 1.01 | 550 |
| 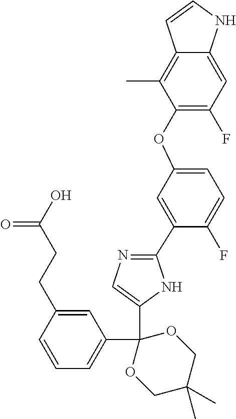 [SMILES: Cc1c(Oc2ccc(F)c(-c3ncc(C4(c5cccc(CCC(=O)O)c5)OCC(C)(C)CO4)[nH]3)c2)c(F)cc2[nH]ccc12] | 230 | 3-(3-(2-(2-(2-fluoro- 5-((6-fluoro-4- methyl-1H-indol-5- yl)oxy)phenyl)-1H- imidazol-5-yl)-5,5- dimethyl-1,3-dioxan- 2- yl)phenyl)propanoic acid | (400 MHz, CD₃OD) δ 7.47-7.39 (m, 3 H), 7.30-7.27 (m, 2H), 7.21-7.09 (m, 4 H), 6.92-6.88 (m, 1 H), 6.52 (d, J = 3.6 Hz, 1H), 3.70 (d, J = 11.2 Hz, 1H), 3.61 (d, J = 11.2 Hz, 1H), 2.92 (t, J = 7.6 Hz, 2H), 2.60 (t, J = 7.6 Hz, 2H), 2.39 (s, 3H), 1.01 (s, 3H), 0.99 (s, 3 H) ppm. | 1.16 | 588 |
| 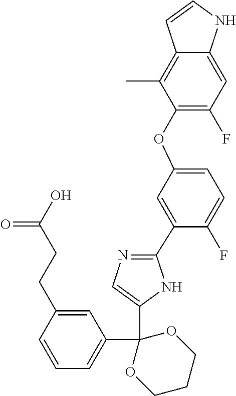 [SMILES: Cc1c(Oc2ccc(F)c(-c3ncc(C4(c5cccc(CCC(=O)O)c5)OCCCO4)[nH]3)c2)c(F)cc2[nH]ccc12] | 231 | 3-(3-(2-(2-(2-fluoro- 5-((6-fluoro-4- methyl-1H-indol-5- yl)oxy)phenyl)-1H- imidazol-4-yl)-1,3- dioxan-2- yl)phenyl)propanoic acid | (400 MHz, CD₃OD) δ 7.48-7.46 (m, 2H), 7.38 (d, J = 7.6 Hz, 1H), 7.30 (d, J = 7.6 Hz, 1H), 7.28 (d, J = 5.6 Hz, 1H), 7.21-7.09 (m, 3H), 6.85 (dt, J = 8.8, 3.6 Hz, 1H), 6.78 (s, 1H), 6.52 (d, J = 2.4, 1H), 4.10-3.96 (m, 4H), 2.95 (t, J = 7.6 Hz, 2H), 2.61 (t, J = 7.6 Hz, 2H), 2.40 (s, 3H), 1.94-1.89 (m, 1H), 1.71-1.67 (m, 1H). | 0.98 | 560 |
| 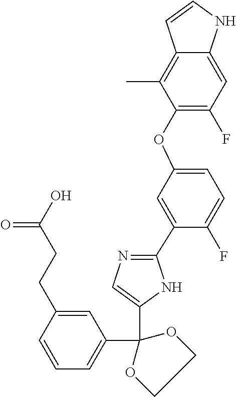 [SMILES: Cc1c(Oc2ccc(F)c(-c3ncc(C4(c5cccc(CCC(=O)O)c5)OCCO4)[nH]3)c2)c(F)cc2[nH]ccc12] | 232 | 3-(3-(2-(2-(2-fluoro- 5-((6-fluoro-4- methyl-1H-indol-5- yl)oxy)phenyl)-1H- imidazol-4-yl)-1,3- dioxolan-2- yl)phenyl)propanoic acid | (400 MHz, CD₃OD) δ 7.48 (m, 2H), 7.39 (m, 1H), 7.30-7.22 (m, 3H), 7.18-7.09 (m, 2H), 6.87 (m, 1H), 6.79 (s, 1H), 6.52 (dd, J = 3.2, 0.8 Hz, 1H), 4.16 (m, 2H), 3.98 (m, 2H), 2.95 (t, J = 7.6 Hz, 2H), 2.61 (t, J = 7.6 Hz, 2H), 2.39 (s, 3H) ppm. | 0.99 | 546 |

### Example 233

### 3-(3-(1-(2-(2-Fluoro-5-((6-fluoro-4-methyl-1H-indol-5-yl)oxy)phenyl)-1H-imidazol-5-yl)-1-(2-(2-methoxyethoxy)ethoxy)ethyl)phenyl)propanoic acid

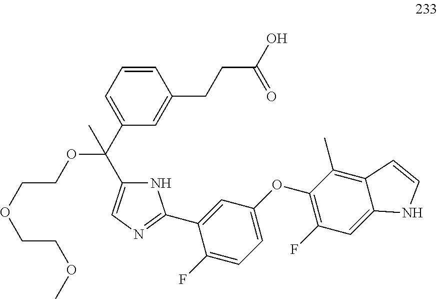 [SMILES: COCCOCCOC(C)(c1cccc(CCC(=O)O)c1)c1cnc(-c2cc(Oc3c(F)cc4[nH]ccc4c3C)ccc2F)[nH]1]

### A. Methyl 3-(3-(1-(2-(2-fluoro-5-((6-fluoro-4-methyl-1H-indol-5-yl)oxy)phenyl)-1H-imidazol-5-yl)-1-hydroxyethyl)phenyl)propanoate

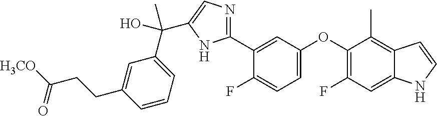 [SMILES: COC(=O)CCc1cccc(C(C)(O)c2cnc(-c3cc(Oc4c(F)cc5[nH]ccc5c4C)ccc3F)[nH]2)c1]

Exchanging methyl 2-fluoro-3-(3-(3-formyloxiran-2-yl)phenyl)propanoate for methyl 3-(3-(3-formyl-2-methyloxiran-2-yl)phenyl)propanoate (692 mg, 2.79 mmol, Intermediate 26) and 3-((4-methyl-1H-indol-5-yl)oxy)benzimidamide for 2-fluoro-5-((6-fluoro-4-methyl-1H-indol-5-yl)oxy)benzimidamide (700 mg, 2.33 mmol, Intermediate 24-10), the reaction conditions described for Step J of Example 172 was used to prepare the title compound (570 mg, 46%) as a light yellow solid. MS: 532 m/z [M+H]⁺.

### B. Methyl 3-(3-(1-(2-(2-fluoro-5-((6-fluoro-4-methyl-1H-indol-5-yl)oxy)phenyl)-1H-imidazol-5-yl)-1-(2-(2-methoxyethoxy)ethoxy)ethyl)phenyl)propanoate

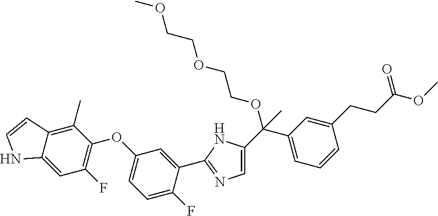 [SMILES: COCCOCCOC(C)(c1cccc(CCC(=O)OC)c1)c1cnc(-c2cc(Oc3c(F)cc4[nH]ccc4c3C)ccc2F)[nH]1]

To a stirred solution of methyl 3-(3-(1-(2-(2-fluoro-5-((6-fluoro-4-methyl-1H-indol-5-yl)oxy)phenyl)-1H-imidazol-5-yl)-1-hydroxyethyl)phenyl)propanoate (100 mg, 0.19 mmol) in 2-(2-methoxyethoxy)ethanol (5 mL) was added PPTS (12 mg, 0.05 mmol). The reaction mixture was stirred room temperature for three days and concentrated. The resulting residue was purified by flash chromatography over silica (petroleum ether/ethyl acetate/dichloromethane, v/v, 2/1/1) to afford the title compound as a light yellow solid (75 mg, 60%). MS: 634 m/z [M+H]⁺.

### C. Example 233. 3-(3-(1-(2-(2-Fluoro-5-((6-fluoro-4-methyl-1H-indol-5-yl)oxy)phenyl)-1H-imidazol-5-yl)-1-(2-(2-methoxyethoxy)ethoxy)ethyl)phenyl)propanoic acid

To a stirred solution of methyl 3-(3-(1-(2-(2-fluoro-5-((6-fluoro-4-methyl-1H-indol-5-yl)oxy)phenyl)-1H-imidazol-5-yl)-1-(2-(2-methoxyethoxy)ethoxy)ethyl)phenyl)propanoate (75 mg, 0.12 mmol) in THF (6 mL) was added lithium hydroxide (18 mg, 0.72 mmol) in water (0.5 mL). The reaction mixture was stirred overnight at room temperature, diluted with water (10 mL), acidified with 1N hydrochloric acid to pH˜4 and extracted with ethyl acetate (30 mL×3). The combined organic extracts were concentrated and the resulting residue was purified by preparative HPLC to afford the title compound as a white solid (19 mg, 26%). MS: 620 m/z [M+H]⁺. ¹H NMR (400 MHz, CD₃OD) δ 7.38-7.35 (m, 2H), 7.27 (d, J=3.6 Hz, 1H), 7.24-7.21 (m, 2H), 7.17-7.09 (m, 3H), 7.02 (s, 1H), 6.87-6.83 (m, 1H), 6.51 (d, J=3.2 Hz, 1H), 3.66-3.61 (m, 4H), 3.53-3.51 (m, 2H), 3.48-3.44 (m, 2H), 3.30 (s, 3H), 2.90 (t, J=7.2 Hz, 2H), 2.56 (t, J=7.2 Hz, 2H), 2.38 (s, 3H), 1.87 (s, 3H) ppm.

The following examples were prepared utilizing similar procedures as described for Example 233.

| | Ex. | | | LCMS | LCMS |
|---|---|---|---|---|---|
| Structure | No. | Name | ¹H NMR | Rₜ (min) | m/z |
| 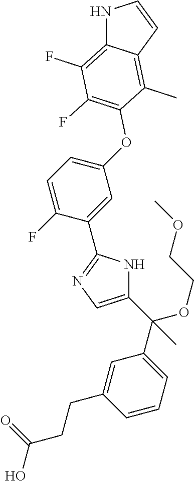 [SMILES: COCCOC(C)(c1cccc(CCC(=O)O)c1)c1cnc(-c2cc(Oc3c(F)c(F)c4[nH]ccc4c3C)ccc2F)[nH]1] | 234 | 3-(3-(1-(2-(5-((6,7- difluoro-4-methyl- 1H-indol-5- yl)oxy)-2- fluorophenyl)-1H- imidazol-5-yl)-1- (2- methoxyethoxy) ethyl)phenyl) propanoic acid | (400 MHz, CD₃OD) δ 7.38 (dd, J = 6.0, 2.8 Hz, 1H) 7.36-7.34 (m, 2H), 7.24-7.13 (m, 4H), 6.98 (s, 1H), 6.90 (dt, J = 8.8, 3.6 Hz, 1H), 6.58 (t, J = 3.2 Hz, 1H), 3.55 (t, J = 4.4 Hz, 2H), 3.45 (t, J = 4.4 Hz, 2H), 3.36 (s, 3H), 2.90 (t, J = 7.6 Hz, 2H), 2.54 (t, J = 7.6 Hz, 2H), 2.35 (s, 3H), 1.87 (s, 3H). ppm. | 1.02 | 594 m/z [M + H]⁺ |
| 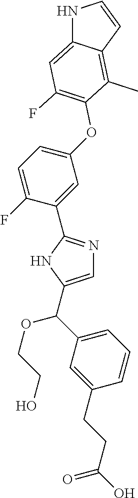 [SMILES: Cc1c(Oc2ccc(F)c(-c3ncc(C(OCCO)c4cccc(CCC(=O)O)c4)[nH]3)c2)c(F)cc2[nH]ccc12] | 235 | 3-(3-((2-(2-fluoro- 5-((6-fluoro-4- methyl-1H-indol-5- yl)oxy)phenyl)-1H- imidazol-5-yl)(2- hydroxyethoxy) methyl)phenyl) propanoic acid | (400 MHz, CD₃OD) δ 7.43 (dd, J = 6.0, 3.2 Hz, 1H), 7.36 (s, 1H), 7.30- 7.27 (m, 3H), 7.23-7.10 (m, 3H), 6.89 (dt, J = 8.8, 3.6 Hz, 1H), 6.69 (s, 1H), 6.52 (dd, J = 3.2, 0.8 Hz, 1H), 5.46 (s, 1H), 3.70 (t, J = 4.8 Hz, 2H), 3.54 (t, J = 4.8 Hz, 2H), 2.94 (t, J = 8.0 Hz, 2H), 2.56 (t, J = 8.0 Hz, 2H), 2.40 (s, 3H) ppm. | 0.87 | 548 m/z [M + H]⁺ |
| 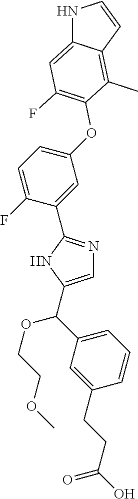 [SMILES: COCCOC(c1cccc(CCC(=O)O)c1)c1cnc(-c2cc(Oc3c(F)cc4[nH]ccc4c3C)ccc2F)[nH]1] | 236 | 3-(3-((2-(2-fluoro- 5-((6-fluoro-4- methyl-1H-indol-5- yl)oxy)phenyl)-1H- imidazol-5-yl)(2- methoxyethoxy) methyl)phenyl) propanoic acid | (400 MHz, CD₃OD) δ 7.42-7.40 (m, 1H), 7.33 (s, 1H), 7.30-7.25 (m, 3H), 7.21-7.10 (m, 3H), 6.91-6.87 (m, 1H), 6.76 (s, 1H), 6.52 (d, J = 2.4 Hz, 1H), 5.46 (s, 1H), 3.65-3.56 (m, 4H), 3.35 (s, 3H), 2.93 (t, J = 7.6 Hz, 2H), 2.53 (t, J = 8.4 Hz, 2H), 2.40 (s, 3H) ppm. | 0.95 | 562 m/z [M + H]⁺ |
| 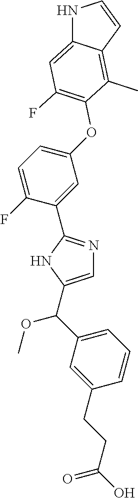 [SMILES: COC(c1cccc(CCC(=O)O)c1)c1cnc(-c2cc(Oc3c(F)cc4[nH]ccc4c3C)ccc2F)[nH]1] | 237 | 3-(3-((2-(2-fluoro- 5-((6-fluoro-4- methyl-1H-indol-5- yl)oxy)phenyl)-1H- imidazol-5- yl)(methoxy) methyl)phenyl) propanoic acid | (400 MHz, CD₃OD) δ 7.44-7.11 (m, 8H), 6.91-6.88 (m, 1H), 6.75 (s, 1H), 6.53 (d, J = 2.8 Hz, 1H), 5.31 (s, 1H), 3.35 (s, 3H), 2.94 (t, J = 7.6 Hz, 2H), 2.60 (t, J = 7.6 Hz, 2H), 2.41 (s, 3H) ppm. | 1.04 | 518 m/z [M + H]⁺ |

### Example 238

### 3-(3-((5-(2-Fluoro-5-((6-fluoro-4-methyl-1H-indol-5-yl)oxy)phenyl)-4H-1,2,4-triazol-3-yl)methyl)phenyl)propanoic acid

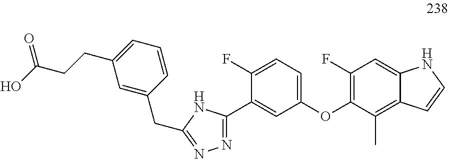 [SMILES: Cc1c(Oc2ccc(F)c(-c3nnc(Cc4cccc(CCC(=O)O)c4)[nH]3)c2)c(F)cc2[nH]ccc12]

### A. 6-Fluoro-5-(4-fluoro-3-(5-(3-iodobenzyl)-4H-1,2,4-triazol-3-yl)phenoxy)-4-methyl-1H-indole

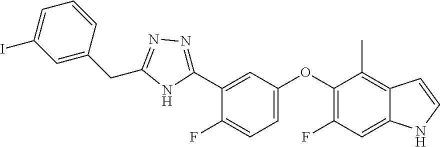 [SMILES: Cc1c(Oc2ccc(F)c(-c3nnc(Cc4cccc(I)c4)[nH]3)c2)c(F)cc2[nH]ccc12]

A mixture of 2-fluoro-5-((6-fluoro-4-methyl-1H-indol-5-yl)oxy)benzohydrazide (350 mg, 1.10 mmol, Intermediate 28), 2-(3-iodophenyl)acetonitrile (402 mg, 1.66 mmol) and potassium carbonate (457 mg, 3.31 mmol) in n-butanol (5 mL) was stirred at 140° C. for four hours under nitrogen atmosphere. The mixture was concentrated and ethyl acetate (50 mL) was added. The organic layer was washed with water (50 mL) and brine (50 mL), dried over sodium sulfate, filtered and concentrated to give a residue which was purified by flash chromatography over silica (petroleum ether/ethyl acetate, v/v, 1/1-1/2) to afford the title compound as a yellow solid (347 mg, 58%). MS: 543 m/z [M+H]⁺.

### B. Ethyl (E)-3-(3-((5-(2-fluoro-5-((6-fluoro-4-methyl-1H-indol-5-yl)oxy)phenyl)-4H-1,2,4-triazol-3-yl)methyl)phenyl)acrylate

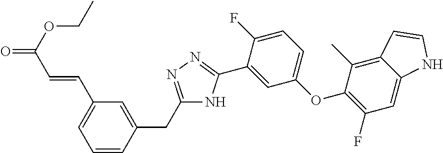 [SMILES: CCOC(=O)/C=C/c1cccc(Cc2nnc(-c3cc(Oc4c(F)cc5[nH]ccc5c4C)ccc3F)[nH]2)c1]

A mixture of 6-fluoro-5-(4-fluoro-3-(5-(3-iodobenzyl)-4H-1,2,4-triazol-3-yl)phenoxy)-4-methyl-1H-indole (347 mg, 0.64 mmol), ethyl acrylate (83 mg, 0.83 mmol), palladium(II) acetate (14 mg, 0.06 mmol), P(o-tolyl)₃ (39 mg, 0.12 mmol) and triethylamine (194 mg, 1.92 mmol) in DMF (8 mL) was heated at 105° C. under nitrogen atmosphere in a microwave synthesizer for 30 minutes. The mixture was filtered through Celite, and the filter cake was washed with ethyl acetate (100 mL). The organic layer was washed with water (30 mL×3) and brine (30 mL×2), dried over sodium sulfate, filtered and concentrated to give a residue, which was purified by flash chromatography over silica (petroleum ether/ethyl acetate, v/v, 1/1 to 1/3) to afford the title compound as a yellow solid (243 mg, 74%). MS: 515 m/z [M+H]⁺.

### C. Ethyl 3-(3-((5-(2-fluoro-5-((6-fluoro-4-methyl-1H-indol-5-yl)oxy)phenyl)-4H-1,2,4-triazol-3-yl)methyl)phenyl)propanoate

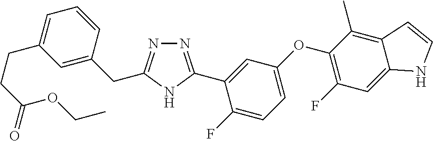 [SMILES: CCOC(=O)CCc1cccc(Cc2nnc(-c3cc(Oc4c(F)cc5[nH]ccc5c4C)ccc3F)[nH]2)c1]

A mixture of ethyl 3-(3-((5-(2-fluoro-5-((6-fluoro-4-methyl-1H-indol-5-yl)oxy)phenyl)-4H-1,2,4-triazol-3-yl)methyl)phenyl)acrylate (243 mg, 0.47 mmol) and 10% Pd/C (100 mg) in methanol (10 mL) was stirred overnight at room temperature under hydrogen atmosphere. The resulting mixture was filtered through Celite and the filtrate was concentrated to afford the title compound as a yellow solid (200 mg, 82%), which was used directly for the next step. MS: 517 m/z [M+H]⁺.

### D. Example 238. 3-(3-((5-(2-Fluoro-5-((6-fluoro-4-methyl-1H-indol-5-yl)oxy)phenyl)-4H-1,2,4-triazol-3-yl)methyl)phenyl)propanoic acid

To a stirred solution of ethyl 3-(3-((5-(2-fluoro-5-((6-fluoro-4-methyl-1H-indol-5-yl)oxy)phenyl)-4H-1,2,4-triazol-3-yl)methyl)phenyl)propanoate (100 mg, 0.19 mmol) in THF (5 mL) was added a solution of lithium hydroxide (23 mg, 0.97 mmol) in water (1 mL). The resulting mixture was stirred at room temperature overnight, diluted with water (10 mL), acidified to pH to 6.5 with 1N hydrochloric acid and extracted with ethyl acetate (20 mL×3). The combined organic phase was washed with water (20 mL) and brine (20 mL), dried over sodium sulfate, filtered and concentrated to give the crude product, which was purified by preparative HPLC to afford the title compound as a white solid (75 mg, 79%). LCMS retention time: 1.19 minutes. MS: 489 m/z [M+H]⁺. ¹H NMR (500 MHz, CD₃OD) δ 7.40 (dd, J=5.5, 3.0 Hz, 1H), 7.28 (d, J=3.5 Hz, 1H), 7.24-7.17 (m, 3H), 7.14-7.08 (m, 3H), 7.01-6.94 (m, 1H), 6.53 (d, J=2.5 Hz, 1H), 4.11 (s, 2H), 2.89 (t, J=8.0 Hz, 2H), 2.55 (t, J=8.0 Hz, 2H), 2.40 (s, 3H) ppm.

The following examples were prepared utilizing the similar procedures as described for Example 238.

| | Ex. | | | LCMS | MS m/z |
|---|---|---|---|---|---|
| Structure | No. | Name | ¹H NMR | Rₜ (min) | [M + H]⁺ |
| 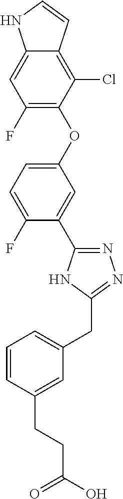 [SMILES: O=C(O)CCc1cccc(Cc2nnc(-c3cc(Oc4c(F)cc5[nH]ccc5c4Cl)ccc3F)[nH]2)c1] | 239 | 3-(3-((5-(5-((4- chloro-6-fluoro- 1H-indol-5- yl)oxy)-2- fluorophenyl)-4H- 1,2,4-triazol-3- yl)methyl)phenyl) propanoic acid | (400 MHz, DMSO-d₆) δ 13.96 (s, 1H), 12.10 (s, 1H), 11.67 (s, 1H), 7.55 (t, J = 2.8 Hz, 1H). 7.48 (d, J = 10.4 Hz, 1H), 7.34-7.28 (m, 2H), 7.21-7.16 (m, 2H), 7.09-7.06 (m, 3H), 6.54 (s, 1H), 4.05 (s, 2H), 2.77 (t, J = 7.6 Hz, 2H), 2.50 (t, J = 7.6 Hz, 2H) ppm. | 1.22 | 509 |
| 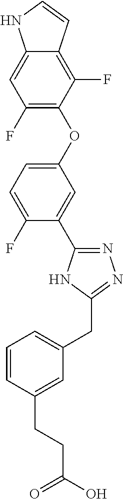 [SMILES: O=C(O)CCc1cccc(Cc2nnc(-c3cc(Oc4c(F)cc5[nH]ccc5c4F)ccc3F)[nH]2)c1] | 240 | 3-(3-((5-(5-((4,6- difluoro-1H-indol- 5-yl)oxy)-2- fluorophenyl)-4H- 1,2,4-triazol-3- yl)methyl)phenyl) propanoic acid | (400 MHz, CD₃OD) δ 7.48 (dd, J = 2.8, 5.6 Hz, 1H), 7.30 (d, J = 3.2 Hz, 1H), 7.05-7.25 (m, 7H), 6.56 (d, J = 3.2 Hz, 1H), 4.10 (s, 2H), 2.88 (t, J = 7.6 Hz, 2H), 2.57 (t, J = 7.6 Hz, 2H) ppm. | 1.17 | 493 |

### Example 241

### 3-(3-((3-(2-Fluoro-5-((6-fluoro-4-methyl-1H-indol-5-yl)oxy)phenyl)-1-methyl-1H-1,2,4-triazol-5-yl)methyl)phenyl)propanoic acid

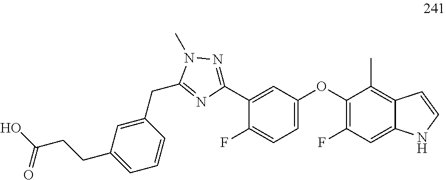 [SMILES: Cc1c(Oc2ccc(F)c(-c3nc(Cc4cccc(CCC(=O)O)c4)n(C)n3)c2)c(F)cc2[nH]ccc12]

### And

### Example 242

### 3-(3-((5-(2-Fluoro-5-((6-fluoro-4-methyl-1H-indol-5-yl)oxy)phenyl)-1-methyl-1H-1,2,4-triazol-3-yl)methyl)phenyl)propanoic acid

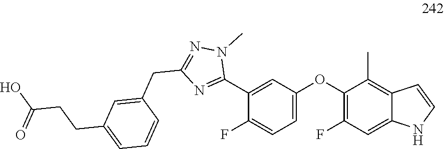 [SMILES: Cc1c(Oc2ccc(F)c(-c3nc(Cc4cccc(CCC(=O)O)c4)nn3C)c2)c(F)cc2[nH]ccc12]

### A. Mixture of ethyl 3-(3-((3-(2-fluoro-5-((6-fluoro-4-methyl-1H-indol-5-yl)oxy)phenyl)-1-methyl-1H-1,2,4-triazol-5-yl)methyl)phenyl)propanoate and ethyl 3-(3-((5-(2-fluoro-5-((6-fluoro-4-methyl-1H-indol-5-yl)oxy)phenyl)-1-methyl-1H-1,2,4-triazol-3-yl)methyl)phenyl)propanoate

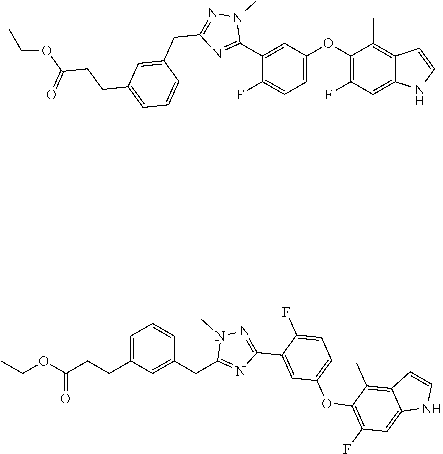 [SMILES: CCOC(=O)CCc1cccc(Cc2nc(-c3cc(Oc4c(F)cc5[nH]ccc5c4C)ccc3F)n(C)n2)c1 ; CCOC(=O)CCc1cccc(Cc2nc(-c3cc(Oc4c(F)cc5[nH]ccc5c4C)ccc3F)nn2C)c1]

To a stirred solution of ethyl 3-(3-((5-(2-fluoro-5-((6-fluoro-4-methyl-1H-indol-5-yl)oxy)phenyl)-4H-1,2,4-triazol-3-yl)methyl)phenyl)propanoate (100 mg, 0.19 mmol, Example 238) and potassium carbonate (53 mg, 0.39 mmol) in DMF (3 mL) was added a solution of iodomethane (28 mg, 0.19 mmol) in DMF (2 mL). The reaction mixture was stirred at room temperature for eight hours, diluted with water (10 mL) and extracted with ethyl acetate (20 mL×3). The combined organic extracts were washed with water (20 mL) and brine (20 mL), dried over sodium sulfate, filtered and concentrated. The resulting residue was purified by flash chromatography over silica (petroleum ether/ethyl acetate, v/v, 1/1-1/3) to afford a mixture of ethyl 3-(3-((3-(2-fluoro-5-((6-fluoro-4-methyl-1H-indol-5-yl)oxy)phenyl)-1-methyl-1H-1,2,4-triazol-5-yl)methyl)phenyl)propanoate and ethyl 3-(3-((5-(2-fluoro-5-((6-fluoro-4-methyl-1H-indol-5-yl)oxy)phenyl)-1-methyl-1H-1,2,4-triazol-3-yl)methyl)phenyl)propanoate as a yellow solid (100 mg, crude). MS: 531 m/z [M+H]⁺.

### B. Example 241: 3-(3-((3-(2-fluoro-5-((6-fluoro-4-methyl-1H-indol-5-yl)oxy)phenyl)-1-methyl-1H-1,2,4-triazol-5-yl)methyl)phenyl)propanoic acid and Example 242: 3-(3-((5-(2-fluoro-5-((6-fluoro-4-methyl-1H-indol-5-yl)oxy)phenyl)-1-methyl-1H-1,2,4-triazol-3-yl)methyl)phenyl)propanoic acid

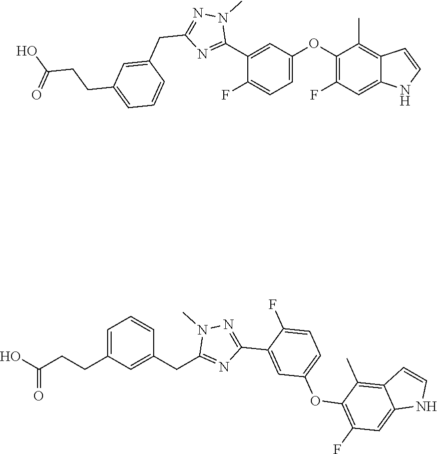 [SMILES: Cc1c(Oc2ccc(F)c(-c3nc(Cc4cccc(CCC(=O)O)c4)nn3C)c2)c(F)cc2[nH]ccc12 ; Cc1c(Oc2ccc(F)c(-c3nc(Cc4cccc(CCC(=O)O)c4)n(C)n3)c2)c(F)cc2[nH]ccc12]

To a stirred solution of ethyl 3-(3-((3-(2-fluoro-5-((6-fluoro-4-methyl-1H-indol-5-yl)oxy)phenyl)-1-methyl-1H-1,2,4-triazol-5-yl)methyl)phenyl)propanoate and ethyl 3-(3-((5-(2-fluoro-5-((6-fluoro-4-methyl-1H-indol-5-yl)oxy)phenyl)-1-methyl-1H-1,2,4-triazol-3-yl)methyl)phenyl)propanoate (100 mg, crude) in THF (5 mL) was added a solution of lithium hydroxide (23 mg, 0.94 mmol) in water (1 mL). The reaction mixture was stirred at room temperature overnight and diluted with water (10 mL). The pH of the resulting mixture was adjusted to ˜4 with 1N hydrochloric acid and then extracted with ethyl acetate (20 mL×3). The combined organic extracts were washed with water (20 mL) and brine (20 mL), dried over sodium sulfate, filtered and concentrated. The resulting residue was purified by preparative HPLC to give di-methylated product 3-(3-((3-(2-fluoro-5-((6-fluoro-1,4-dimethyl-1H-indol-5-yl)oxy)phenyl)-1-methyl-1H-1,2,4-triazol-5-yl)methyl)phenyl)propanoic acid (27 mg, 27% from 2 steps) and a mixture of mono-methylated products 3-(3-((3-(2-fluoro-5-((6-fluoro-4-methyl-1H-indol-5-yl)oxy)phenyl)-1-methyl-1H-1,2,4-triazol-5-yl)methyl)phenyl)propanoic acid and 3-(3-((5-(2-fluoro-5-((6-fluoro-4-methyl-1H-indol-5-yl)oxy)phenyl)-1-methyl-1H-1,2,4-triazol-3-yl)methyl)phenyl)propanoic acid. Dimethylated product—3-(3-((3-(2-Fluoro-5-((6-fluoro-1,4-dimethyl-1H-indol-5-yl)oxy)phenyl)-1-methyl-1H-1,2,4-triazol-5-yl)methyl)phenyl)propanoic acid: ¹H NMR (400 MHz, CD₃OD) δ 7.35 (dd, J=5.6, 3.2 Hz, 1H), 7.29-7.11 (m, 6H), 7.05 (d, J=8.0 Hz, 1H), 6.98-6.91 (m, 1H), 6.51 (d, J=3.2 Hz, 1H), 4.23 (s, 2H), 3.80 (s, 3H), 3.76 (s, 3H), 2.89 (t, J=7.6 Hz, 2H), 2.58 (t, J=7.6 Hz, 2H), 2.39 (s, 3H) ppm. MS: 517 m/z [M+H]⁺.

The mono-methylated products were further separated by SFC to give Example 241 and Example 242.

Example 241. 3-(3-((3-(2-Fluoro-5-((6-fluoro-4-methyl-1H-indol-5-yl)oxy)phenyl)-1-methyl-1H-1,2,4-triazol-5-yl)methyl)phenyl)propanoic acid was obtained as a white solid (24 mg, 25%). MS: 503 m/z [M+H]⁺. ¹H NMR (400 MHz, CD₃OD) δ 7.37 (dd, J=5.6, 3.2 Hz, 1H), 7.30-7.09 (m, 6H), 7.04 (d, J=7.2 Hz, 1H), 6.98-6.91 (m, 1H), 6.53 (d, J=3.2 Hz, 1H), 4.22 (s, 2H), 3.76 (s, 3H), 2.89 (t, J=7.6 Hz, 2H), 2.56 (t, J=7.6 Hz, 2H), 2.40 (s, 3H) ppm.

Example 242. 3-(3-((5-(2-Fluoro-5-((6-fluoro-4-methyl-1H-indol-5-yl)oxy)phenyl)-1-methyl-1H-1,2,4-triazol-3-yl)methyl)phenyl)propanoic acid was obtained as a white solid (9.6 mg, 10%). MS: 503 m/z [M+H]⁺. ¹H NMR (400 MHz, CD₃OD) δ 7.29-7.26 (m, 2H), 7.21-7.16 (m, 2H), 7.13-7.07 (m, 4H), 6.99 (dd, J=3.2, 5.6 Hz, 1H), 6.52 (d, J=3.2 Hz, 1H), 3.99 (s, 2H), 3.79 (d, J=2.0 Hz, 3H), 2.87 (t, J=7.6 Hz, 2H), 2.55 (t, J=7.6 Hz, 2H), 2.39 (s, 3H) ppm.

### Example 243

### rac-(1R,2R)-2-(3-((5-(3-((4-Methyl-1H-indol-5-yl)oxy)phenyl)-4H-1,2,4-triazol-3-yl)methyl)phenyl)cyclopropane-1-carboxylic acid

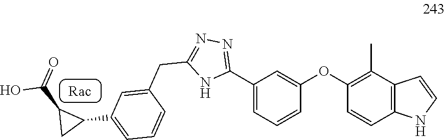 [SMILES: Cc1c(Oc2cccc(-c3nnc(Cc4cccc([C@@H]5C[C@H]5C(=O)O)c4)[nH]3)c2)ccc2[nH]ccc12]

### A. 2-(3-Vinylphenyl)acetonitrile

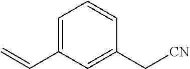

A mixture of 2-(3-bromophenyl)acetonitrile (3.92 g, 20 mmol), 4,4,5,5-tetramethyl-2-vinyl-1,3,2-dioxaborolane (6.16 g, 40 mg), Pd(dppf)Cl₂ (1.46 g, 2 mmol) and cesium carbonate (19.5 g, 60 mmol) in 1,4-dioxane (200 mL) and water (20 mL) was stirred at 100° C. for six hours under nitrogen atmosphere The reaction mixture was cooled to room temperature, filtered and concentrated. The resulting residue was purified by flash chromatography over silica (petroleum ether/ethyl acetate, v/v, 16/1 to 4/1) to afford the title compound as a yellow oil (2.25 g, 78%). ¹H NMR (400 MHz, CDCl₃) δ 7.39-7.32 (m, 3H), 7.22-7.20 (m, 1H), 6.64-6.67 (dd, J=17.6 Hz, 1H), 5.78 (d, J=17.6, 10.8 Hz, 1H), 5.31 (d, J=7.2, 10.8 Hz, 1H), 3.76 (s, 2H) ppm

### B. rac-Ethyl (1R,2R)-2-(3-(cyanomethyl)phenyl)cyclopropane-1-carboxylate

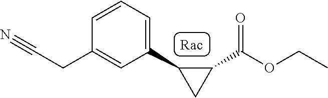 [SMILES: CCOC(=O)[C@@H]1C[C@H]1c1cccc(CC#N)c1]

To a stirred solution of copper(II) acetylacetonate (261 mg, 1 mmol) in anhydrous dichloromethane (80 mL) was added 2-(3-vinylphenyl)acetonitrile (1.43 g, 10 mmol) and the reaction mixture was stirred at 40° C. for five minutes. A solution of ethyl diazoacetate (1.56 mL, 15 mmol) in dichloromethane (20 mL) was added over eight hours and continued stirring for one more hour after the addition. The reaction mixture was diluted with dichloromethane (100 mL), washed with saturated sodium bicarbonate solution (60 mL×2), water (30 mL×2) and brine, dried over sodium sulfate and concentrated. The resulting residue was separated by silica gel chromatography (10% ethyl acetate in petroleum ether) to afford the title compound as colorless oil (1.24 g, 54%). ¹H NMR (400 MHz, CDCl3) δ 7.31-7.26 (m, 1H), 7.17-7.15 (m, 1H), 7.07 (s, 1H), 7.06-7.04 (m, 1H), 4.17 (q, J=7.2 Hz, 2H), 3.72 (s, 2H), 2.53-2.50 (m, 1H), 1.92-1.88 (m, 1H), 1.64-1.59 (m, 1H), 1.31-1.27 (m, 1H), 1.29 (t, J=7.2 Hz, 3H) ppm.

### C. rac-(1R,2R)-2-(3-(Cyanomethyl)phenyl)cyclopropane-1-carboxylic acid

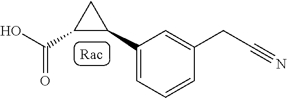 [SMILES: N#CCc1cccc([C@@H]2C[C@H]2C(=O)O)c1]

To a stirred solution of rac-ethyl (1R,2R)-2-(3-(cyanomethyl)phenyl)cyclopropane-1-carboxylate (1.15 g, 5 mmol) in THF (20 mL) was added a solution of lithium hydroxide (1.2 g, 50 mmol) in water (5 mL). The reaction mixture was stirred at room temperature for six hours and then concentrated to remove THF. The pH of the mixture was adjusted to 4˜5 with 1N hydrochloric acid and extracted with ethyl acetate. The combined organic extracts were washed with brine, dried over sodium sulfate and concentrated to afford the title compound as a white solid (950 mg, 95%). MS: 202 m/z [M+H]⁺.

### D. Example 243: rac-(1R,2R)-2-(3-((5-(3-((4-Methyl-1H-indol-5-yl)oxy)phenyl)-4H-1,2,4-triazol-3-yl)methyl)phenyl)cyclopropane-1-carboxylic acid

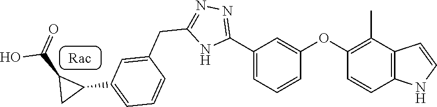 [SMILES: Cc1c(Oc2cccc(-c3nnc(Cc4cccc([C@@H]5C[C@H]5C(=O)O)c4)[nH]3)c2)ccc2[nH]ccc12]

A mixture of rac-(1R,2R)-2-(3-(cyanomethyl)phenyl)cyclopropane-1-carboxylic acid (99 mg, 0.50 mmol) and 3-((4-methyl-1H-indol-5-yl)oxy)benzohydrazide (281 mg, 1 mmol, Intermediate 28-2), and potassium carbonate (276 mg, 2.0 mmol) in n-butanol (2 mL) was heated at 150° C. in the microwave reactor for four hours. After removal of the solvent, the residue was purified by preparative HPLC to afford the title compound as a white solid (4.1 mg, 1.8%). LC-MS retention time: 1.18 minutes. MS: 465 m/z [M+H]⁺. ¹H NMR (400 MHz, DMSO-d₆) δ 11.22 (s, 1H), 7.59-7.57 (m, 1H), 7.40-7.35 (m, 2H), 7.30-7.27 (m, 2H), 7.20-7.16 (m, 1H), 7.09-7.05 (m, 2H), 7.01-6.99 (m, 1H), 6.93-6.90 (m, 1H), 6.82-6.80 (m, 1H), 6.50 (m, 1H), 4.01 (s, 2H), 2.33-2.31 (m, 1H), 2.28 (s, 3H), 1.77-1.75 (m, 1H), 1.40-1.38 (m, 1H), 1.27-1.25 (m, 1H) ppm.

### Example 244

### 4-((5-(2-Fluoro-5-((6-fluoro-4-methyl-1H-indol-5-yl)oxy)phenyl)-4H-1,2,4-triazol-3-yl)methyl)-2,3-dihydro-1H-indene-2-carboxylic acid

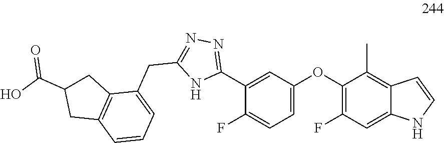 [SMILES: Cc1c(Oc2ccc(F)c(-c3nnc(Cc4cccc5c4CC(C(=O)O)C5)[nH]3)c2)c(F)cc2[nH]ccc12]

### A. Methyl 4-bromo-1-oxo-2,3-dihydro-1H-indene-2-carboxylate

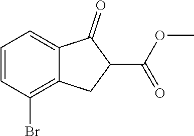

To a stirred solution of 4-bromo-2,3-dihydro-1H-inden-1-one (5 g, 23.7 mmol) in THF (20 mL) was added lithium bis(trimethylsilyl)amide (59.2 mL, 59.2 mmol, 1 N in THF) at 0° C. The reaction mixture was slowly warmed to room temperature and stirred for thirty minutes. A solution of methyl carbonochloridate (3.3 g, 35.5 mmol) in THF (15 mL) was then added at 0° C., the reaction mixture was warmed to room temperature and stirred for two hours. The reaction was quenched with hydrochloric acid (1N, 30 mL) and extracted with ethyl acetate (100 mL×3). The combined organic phase was washed with water and brine, dried over sodium sulfate, filtered and concentrated. The resulting residue was purified by flash chromatography over silica (10% ethyl acetate in petroleum ether) to afford the title compound as a yellow solid (1.8 g, 28%). ¹H NMR (400 MHz, CD₃OD) δ 7.92 (d, J=7.6 Hz, 1H), 7.73 (d, J=7.6 Hz, 1H), 7.62 (dd, J=7.6, 0.8 Hz, 1H), 7.44-7.34 (m, 1H), 3.78 (s, 3H), 3.39 (s, 2H) ppm.

### B. Methyl 4-bromo-2,3-dihydro-1H-indene-2-carboxylate

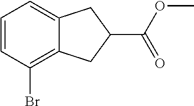

To a stirred solution of methyl 4-bromo-1-oxo-2,3-dihydro-1H-indene-2-carboxylate (1.8 g, 6.69 mmol) in trifluoroacetic acid (30 mL) was added triethylsilane (6.6 g, 40.1 mmol) at 0° C. The reaction mixture was stirred at room temperature overnight, quenched with saturated ammonium chloride solution (15 mL) and concentrated. The resulting residue was dissolved in ethyl acetate (100 mL), washed with brine (20 mL×3), dried over sodium sulfate, filtered and concentrated. The crude product was purified by flash chromatography over silica (petroleum ether/ethyl acetate, v/v, 8/1) to afford the title compound as a yellow oil (382 mg, 22%). MS: 255 m/z [M+H]⁺. ¹H NMR (400 MHz, CDCl₃) δ 7.34 (d, J=8.0 Hz, 1H), 7.16 (d, J=7.2 Hz, 1H), 7.07-7.03 (m, 1H), 3.76 (s, 3H), 2.40-3.27 (m, 5H) ppm.

### C. Methyl 4-(cyanomethyl)-2,3-dihydro-1H-indene-2-carboxylate

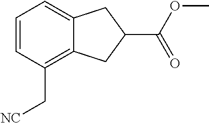 [SMILES: COC(=O)C1Cc2cccc(CC#N)c2C1]

A mixture of 4-bromo-2,3-dihydro-1H-indene-2-carboxylate (130 mg, 0.51 mmol), 2-(trimethylsilyl)acetonitrile (87 mg, 0.77 mmol), zinc fluoride (26 mg, 0.26 mmol), Pd₂(dba)₃ (15 mg, 0.02 mmol) and Xantphos (9 mg, 0.02 mmol) in DMF (15 mL) was stirred overnight at 90° C. under nitrogen atmosphere. Ethyl acetate (100 mL) was added and the mixture was washed with water (40 mL×4) and brine (40 mL×2), dried over sodium sulfate and concentrated. The resulting crude product was purified by preparative TLC (petroleum ether/ethyl acetate, v/v, 6/1) to afford the title compound as a light yellow solid (60 mg, 54%). MS: 216 m/z [M+H]⁺.

### D. Example 244. 4-((5-(2-Fluoro-5-((6-fluoro-4-methyl-1H-indol-5-yl)oxy)phenyl)-4H-1,2,4-triazol-3-yl)methyl)-2,3-dihydro-1H-indene-2-carboxylic acid

A mixture of methyl 4-(cyanomethyl)-2,3-dihydro-1H-indene-2-carboxylate (60 mg, 0.28 mmol), 2-fluoro-5-((6-fluoro-4-methyl-1H-indol-5-yl)oxy)benzohydrazide (88 mg, 0.28 mmol, Intermediate 28) and potassium carbonate (77 mg, 0.56 mmol) in n-butanol (10 mL) was stirred in a microwave synthesizer at 160° C. for eight hours. The reaction mixture was concentrated and the resulting residue was purified by preparative HPLC to afford the title compound as a white solid (26 mg, 18%). MS: 501 m/z [M+H]⁺. ¹H NMR (400 MHz, CD₃OD) δ 7.41 (s, 1H), 7.27 (d, J=3.2 Hz, 1H), 7.19 (t, J=9.6 Hz, 1H), 7.14-7.10 (m, 3H), 7.01-6.95 (m, 2H), 6.52 (d, J=2.8 Hz, 1H), 4.11 (s, 2H), 3.30-3.16 (m, 5H), 2.39 (s, 3H) ppm.

### Example 245

### 5-((5-(2-Fluoro-5-((6-fluoro-4-methyl-1H-indol-5-yl)oxy)phenyl)-4H-1,2,4-triazol-3-yl)methyl)-2,3-dihydro-1H-indene-2-carboxylic acid

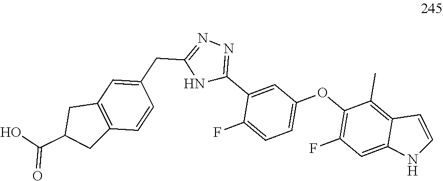 [SMILES: Cc1c(Oc2ccc(F)c(-c3nnc(Cc4ccc5c(c4)CC(C(=O)O)C5)[nH]3)c2)c(F)cc2[nH]ccc12]

### A. Methyl 5-vinyl-2,3-dihydro-1H-indene-2-carboxylate

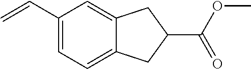

To a stirred solution of methyl 5-bromo-2,3-dihydro-1H-indene-2-carboxylate (342 mg, 2.22 mmol) and 4,4,5,5-tetramethyl-2-vinyl-1,3,2-dioxaborolane (378 mg, 1.48 mmol) in THF (20 mL) were added Pd(PPh₃)₄ (137 mg, 0.12 mmol) and cesium carbonate (1.92 g, 5.92 mmol). The reaction mixture was refluxed under nitrogen atmosphere overnight, cooled to room temperature, quenched with water (30 mL) and extracted with ethyl acetate (30 mL×3). The combined organic extracts were washed with brine (20 mL), dried over sodium sulfate, filtered and concentrated. The resulting residue was purified by flash chromatography (5% ethyl acetate in petroleum ether) to afford the title compound as a yellow oil (260 mg, 80%). MS: 203 m/z [M+H]⁺.

### B. 5-Formyl-2,3-dihydro-1H-indene-2-carboxylate

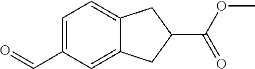

To a stirred solution of methyl 5-vinyl-2,3-dihydro-1H-indene-2-carboxylate (500 mg, 2.48 mmol) in THF (30 mL) and water (10 mL) was added three drops of osmium tetroxide, followed by sodium periodate (1.59 g, 7.43 mmol). The reaction mixture was stirred at room temperature for six hours, quenched with saturated aqueous sodium thiosulfate (30 mL) and extracted with ethyl acetate (20 mL×3). The combined organic phase was washed with brine (10 mL), dried over sodium sulfate, filtered and concentrated to afford the title compound as yellow oil (500 mg, crude), which was used directly in the next step without further purification. MS: 205 m/z [M+H]⁺.

### C. Methyl 5-(hydroxymethyl)-2,3-dihydro-1H-indene-2-carboxylate

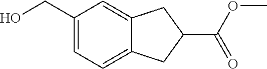

To a stirred solution of methyl 5-formyl-2,3-dihydro-1H-indene-2-carboxylate (500 mg, 2.45 mmol) in methanol (5 mL) was added sodium borohydride (279 mg, 7.35 mmol) at 0° C. The reaction mixture was stirred at room temperature for two hours, quenched with saturated ammonium chloride solution (30 mL) and extracted with ethyl acetate (20 mL×3). The combined organic extracts were washed with brine (10 mL), dried over sodium sulfate, filtered and concentrated. The resulting residue was purified by flash chromatography (20% ethyl acetate in petroleum ether) to afford the title compound as a colorless oil (200 mg, 39% from 2 steps). MS: 207 m/z [M+H]⁺.

### D. Methyl 5-(((methylsulfonyl)oxy)methyl)-2,3-dihydro-1H-indene-2-carboxylate

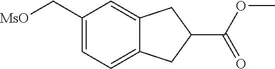 [SMILES: COC(=O)C1Cc2ccc(COS(C)(=O)=O)cc2C1]

A mixture of methyl 5-(hydroxymethyl)-2,3-dihydro-1H-indene-2-carboxylate (200 mg, 0.97 mmol), methanesulfonyl chloride (166 mg, 1.46 mmol) and triethylamine (294 mg, 2.91 mmol) in dichloromethane (30 mL) was stirred at room temperature for six hours, quenched with aqueous saturated sodium bicarbonate (30 mL) and extracted with dichloromethane (20 mL×2). The combined organic extracts were washed with brine (10 mL), dried over sodium sulfate, filtered and concentrated to afford the title compound as a light yellow solid (270 mg, crude), which was used directly in the next step.

### E. Methyl 5-(cyanomethyl)-2,3-dihydro-1H-indene-2-carboxylate

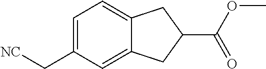 [SMILES: COC(=O)C1Cc2ccc(CC#N)cc2C1]

A mixture of methyl 5-(((methylsulfonyl)oxy)methyl)-2,3-dihydro-1H-indene-2-carboxylate (270 mg, 0.95 mmol) and potassium cyanide (124 mg, 1.90 mmol) in DMF (3 mL) was stirred at 65° C. for two hours, cooled to room temperature and diluted with ethyl acetate (60 mL). The organic phase was washed with water (15 mL×2) and brine (15 mL×2), dried over sodium sulfate, filtered and concentrated. The resulting residue was purified by preparative TLC (developed with petroleum ether/ethyl acetate, v/v, 8/1) to afford the title compound as a colorless oil (60 mg, 29% from 2 steps). ¹H NMR (400 MHz, CDCl₃) δ 7.26-7.18 (m, 2H), 7.11 (d, J=7.6 Hz, 1H), 3.73 (s, 3H), 3.7 (s, 2H), 3.36 (m, 1H), 3.25-3.20 (m, 4H) ppm.

### F. Example 245. 5-((5-(2-Fluoro-5-((6-fluoro-4-methyl-1H-indol-5-yl)oxy)phenyl)-4H-1,2,4-triazol-3-yl)methyl)-2,3-dihydro-1H-indene-2-carboxylic acid

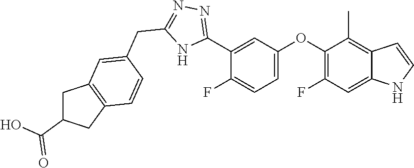 [SMILES: Cc1c(Oc2ccc(F)c(-c3nnc(Cc4ccc5c(c4)CC(C(=O)O)C5)[nH]3)c2)c(F)cc2[nH]ccc12]

A mixture of methyl 5-(cyanomethyl)-2,3-dihydro-1H-indene-2-carboxylate (60 mg, 0.28 mmol), 2-fluoro-5-((6-fluoro-4-methyl-1H-indol-5-yl)oxy)benzohydrazide (59 mg, 0.19 mmol, Intermediate 28) and potassium carbonate (52 mg, 0.38 mmol) in n-butanol (2 mL) was stirred at 160° C. for three hours under nitrogen atmosphere in a microwave reactor and then concentrated to remove n-butanol. The resulting residue was diluted with ethyl acetate (30 mL), washed with water (10 mL) and brine (10 mL), dried over sodium sulfate, filtered and concentrated. The crude product was purified by preparative HPLC to afford the title compound as a white solid (20 mg, 22%). MS: 501 m/z [M+H]⁺. ¹H NMR (400 MHz, CD₃OD) δ 7.38-7.37 (m, 1H), 7.28 (d, J=3.2 Hz, 1H), 7.19 (t, J=9.6 Hz, 1H), 7.13-7.10 (m, 4H), 7.06-7.04 (m, 1H), 6.53 (d, J=3.2 Hz, 1H), 4.09 (s, 2H), 3.33-3.26 (m, 1H), 3.17-3.15 (m, 4H), 2.39 (s, 3H) ppm.

The following example was prepared utilizing similar procedures as described for Example 245.

| | Ex. | | | LCMS | MS m/z |
|---|---|---|---|---|---|
| Structure | No. | Name | ¹H NMR | Rₜ (min) | [M + H]⁺ |
| 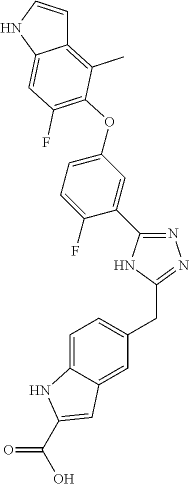 [SMILES: Cc1c(Oc2ccc(F)c(-c3nnc(Cc4ccc5[nH]c(C(=O)O)cc5c4)[nH]3)c2)c(F)cc2[nH]ccc12] | 246 | 5-((5-(2-fluoro-5-((6- fluoro-4-methyl-1H- indol-5- yl)oxy)phenyl)-4H- 1,2,4-triazol-3- yl)methyl)-1H- indole-2-carboxylic acid | (400 MHz, DMSO-d₆) δ 11.40 (s, 1H), 7.45 (s, 1H), 7.40-7.39 (m, 1H), 7.32-7.21 (m, 4H), 7.06 (d, J = 8.4 Hz, 1H), 7.01-6.98 (m, 1H), 6.83 (s, 1H), 6.52 (s, 1H), 4.10 (s, 2H), 2.32 (s, 3H) ppm. | 1.15 | 500 |

### Example 247

### 3-(3-((5-(2-Fluoro-5-((6-fluoro-4-methyl-1H-indol-5-yl)oxy)phenyl)-4H-1,2,4-triazol-3-yl)methyl)phenyl)cyclobutane-1-carboxylic acid

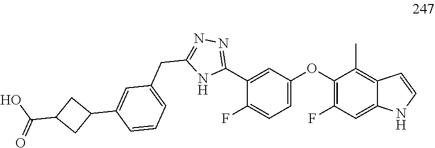 [SMILES: Cc1c(Oc2ccc(F)c(-c3nnc(Cc4cccc(C5CC(C(=O)O)C5)c4)[nH]3)c2)c(F)cc2[nH]ccc12]

### A. Methyl 3-(3-bromophenyl)-3-hydroxycyclobutane-1-carboxylate

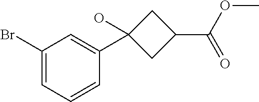 [SMILES: COC(=O)C1CC(O)(c2cccc(Br)c2)C1]

To a stirred solution of 1-bromo-3-iodobenzene (10.0 g, 35.3 mmol) in dry THF (100 mL) was added isopropylmagnesium chloride lithium chloride complex (1.3 M in THF, 33 mL, 42.4 mmol) dropwise at 0° C. After stirring for one hour, the mixture was added dropwise into a solution of methyl 3-oxocyclobutane-1-carboxylate (4.52 g, 35.3 mmol) in THF (50 mL). The reaction mixture was stirred for another hour, quenched with saturated ammonium chloride solution (100 mL) and extracted with ethyl acetate (100 mL×3). The combined organic extracts were washed with brine (100 mL), dried over sodium sulfate, filtered and concentrated. The resulting residue was purified by flash chromatography (0-50% ethyl acetate in petroleum ether) to afford the title compound as an oil (3.7 g, 37%). MS: 307 m/z [M+Na]⁺.

### B. Methyl 3-(3-bromophenyl)cyclobutane-1-carboxylate

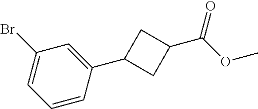

To a stirred solution of methyl 3-(3-bromophenyl)-3-hydroxycyclobutane-1-carboxylate (1.8 g, 6.3 mmol) in trifluoroacetic acid (20 mL) was added triethylsilane (4.38 g, 37.8 mmol) dropwise, at 0° C., stirred at room temperature overnight and concentrated. Ethyl acetate (100 mL) was added to the residue, washed with saturated sodium bicarbonate solution (50 mL) and brine (50 mL), dried over sodium sulfate, filtered and concentrated. The resulting residue was purified by flash chromatography (0-50% ethyl acetate in petroleum ether) to afford the title compound as an oil (1.3 g, 76%). MS: 269, 271 m/z [M+H]⁺.

### C. Methyl 3-(3-(cyanomethyl)phenyl)cyclobutane-1-carboxylate

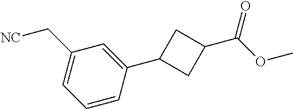 [SMILES: COC(=O)C1CC(c2cccc(CC#N)c2)C1]

To a stirred solution of methyl 3-(3-bromophenyl)cyclobutane-1-carboxylate (2.0 g, 7.46 mmol) and 2-(trimethylsilyl)acetonitrile (1.2 g, 11.2 mmol) in DMF (12 mL) were added Pd₂(dba)₃ (205 mg, 0.22 mmol), Xantphos (127 mg, 0.22 mmol) and zinc fluoride (384 mg, 3.73 mmol). The mixture was stirred at 90° C. under nitrogen for eighteen hours, cooled to room temperature, quenched with water (30 mL) and extracted with ethyl acetate (50 mL×2). The combined organic extracts were washed with brine (50 mL×2), dried over sodium sulfate, filtered and concentrated. The residue was purified by flash chromatography (0-35% ethyl acetate in petroleum ether) to afford the title compound as a yellow oil (520 mg, 24%). MS: 230 m/z [M+H]⁺.

### D. Butyl 3-(3-((5-(2-fluoro-5-((6-fluoro-4-methyl-1H-indol-5-yl)oxy)phenyl)-4H-1,2,4-triazol-3-yl)methyl)phenyl)cyclobutane-1-carboxylate

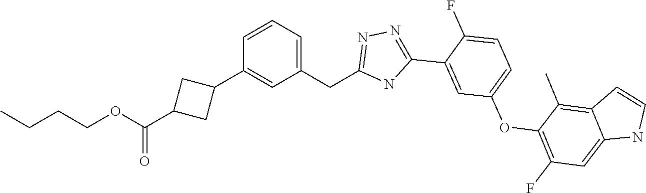 [SMILES: CCCCOC(=O)C1CC(c2cccc(Cc3nnc(-c4cc(Oc5c(F)cc6[nH]ccc6c5C)ccc4F)[nH]3)c2)C1]

To a stirred solution of 2-fluoro-5-((6-fluoro-4-methyl-1H-indol-5-yl)oxy)benzohydrazide (200 mg, 0.63 mmol, Intermediate 28) and methyl 3-(3-(cyanomethyl)phenyl)cyclobutane-1-carboxylate (217 mg, 0.95 mmol) in n-butanol (3 mL) was added potassium carbonate (174 mg, 1.26 mmol). The reaction mixture was stirred at 130° C. in a microwave reactor for three hours, diluted with water (30 mL) and extracted with ethyl acetate (40 mL×2). The combined organic extracts were washed with brine (40 mL×2), dried over sodium sulfate, filtered and concentrated. The resulting residue was purified by flash chromatography (35-50% ethyl acetate in petroleum ether) to afford the title compound as a yellow solid (40 mg, 11%). MS: 571 m/z [M+H]⁺.

### E. Example 247. 3-(3-((5-(2-Fluoro-5-((6-fluoro-4-methyl-1H-indol-5-yl)oxy)phenyl)-4H-1,2,4-triazol-3-yl)methyl)phenyl)cyclobutane-1-carboxylic acid

To a stirred solution of butyl 3-(3-((5-(2-fluoro-5-((6-fluoro-4-methyl-1H-indol-5-yl)oxy)phenyl)-4H-1,2,4-triazol-3-yl)methyl)phenyl)cyclobutane-1-carboxylate (40 mg, 0.07 mmol) in a 2:1 mixture of THF and methanol (3 mL) was added lithium hydroxide (1M in water, 1 mL, 1 mmol). The reaction mixture was stirred at room temperature for two hours and concentrated below 25° C. to remove THF and methanol. The aqueous solution was cooled to 0° C., acidified with hydrochloric acid (1 M) to pH 4 and extracted with ethyl acetate (30 mL×2). The combined organic extracts were washed with brine (40 mL), dried over sodium sulfate, filtered and concentrated. The resulting residue was purified by flash chromatography twice (5% methanol in dichloromethane) to afford the title compound as a white solid (8 mg, 22%). MS: 515 m/z [M+H]⁺. ¹H NMR (400 MHz, CD₃OD) δ 7.39 (s, 1H), 7.31-7.08 (m, 7H), 6.98 (s, 1H), 6.53 (d, J=2.8 Hz, 1H), 4.13 (s, 2H), 3.56-3.38 (m, 1H), 3.20-3.05 (m, 1H), 2.72-2.52 (m, 2H), 2.40 (s, 3H), 2.38-2.25 (m, 2H) ppm.

### Example 248

### 3-Fluoro-3-(3-((5-(2-fluoro-5-((6-fluoro-4-methyl-1H-indol-5-yl)oxy)phenyl)-4H-1,2,4-triazol-3-yl)methyl)phenyl)cyclobutane-1-carboxylic acid

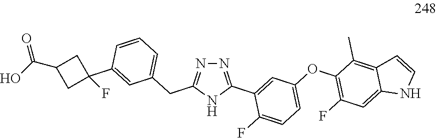 [SMILES: Cc1c(Oc2ccc(F)c(-c3nnc(Cc4cccc(C5(F)CC(C(=O)O)C5)c4)[nH]3)c2)c(F)cc2[nH]ccc12]

### A. Methyl 3-(3-bromophenyl)-3-fluorocyclobutane-1-carboxylate

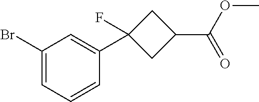 [SMILES: COC(=O)C1CC(F)(c2cccc(Br)c2)C1]

To a stirred solution of methyl 3-(3-bromophenyl)-3-hydroxycyclobutane-1-carboxylate (2.8 g, 9.82 mmol, Example 247, Step A) in dichloromethane (20 mL) was added Morpho-DAST (1.32 mL, 10.8 mmol) dropwise at 0° C. The reaction mixture was stirred at room temperature for two hours, quenched with water (30 mL) and extracted with ethyl acetate (50 mL×2). The combined organic extracts were washed with brine (50 ml×2), dried over magnesium sulfate and concentrated. The resulting residue was purified by chromatography on silica gel (petroleum ether/ethyl acetate, v/v, 20/1) to afford the title compound as a yellow oil (2.0 g, 71%). MS: 287 m/z [M+H]⁺. ¹H NMR (400 MHz, CDCl₃) δ 7.59 (d, J=9.5 Hz, 1H), 7.51-7.43 (m, 1H), 7.38 (t, J=7.5 Hz, 1H), 7.31-7.24 (m, 1H), 3.74 (s, 3H), 3.56-344 (m, 1H), 2.90-277 (m, 4H) ppm.

### B. Methyl 3-(3-(cyanomethyl)phenyl)-3-fluorocyclobutane-1-carboxylate

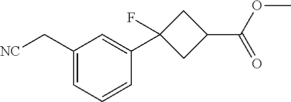 [SMILES: COC(=O)C1CC(F)(c2cccc(CC#N)c2)C1]

To a stirred solution of methyl 3-(3-bromophenyl)-3-fluorocyclobutane-1-carboxylate (1.0 g, 3.5 mmol) and 2-(trimethylsilyl)acetonitrile (593 mg, 5.2 mmol) in DMF (6 mL) were added Pd₂(dba)₃ (96 mg, 0.1 mmol), Xantphos (58 mg, 0.10 mmol) and zinc fluoride (180 mg, 1.75 mmol). The reaction mixture was purged with nitrogen, stirred at 90° C. for eighteen hours under nitrogen, quenched with water (30 mL) and extracted with ethyl acetate (40 mL×2). The organic extracts were washed with brine (40 mL×2), dried over sodium sulfate, filtered and concentrated. The resulting residue was purified by flash chromatography (0-35% ethyl acetate in petroleum ether) to give the title compound as a yellow oil (500 mg, 57%). MS: 248 m/z [M+H]⁺. ¹H NMR (400 MHz, CDCl₃) δ 7.44-7.36 (m, 3H), 7.35-7.29 (m, 1H), 3.78 (s, 2H), 3.73 (s, 3H), 3.57-3.45 (m, 1H), 2.86 (dd, J=24.3, 8.7 Hz, 4H) ppm.

### C. Butyl 3-fluoro-3-(3-((5-(2-fluoro-5-((6-fluoro-4-methyl-1H-indol-5-yl)oxy)phenyl)-4H-1,2,4-triazol-3-yl)methyl)phenyl)cyclobutane-1-carboxylate

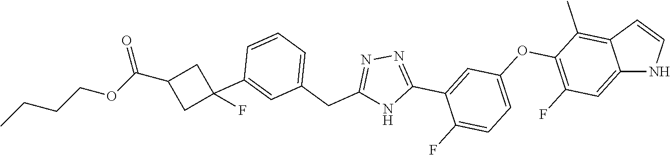 [SMILES: CCCCOC(=O)C1CC(F)(c2cccc(Cc3nnc(-c4cc(Oc5c(F)cc6[nH]ccc6c5C)ccc4F)[nH]3)c2)C1]

To a stirred solution of 2-fluoro-5-((6-fluoro-4-methyl-1H-indol-5-yl)oxy)benzohydrazide (200 mg, 0.63 mmol, Intermediate 28) and methyl 3-(3-(cyanomethyl)phenyl)-3-fluorocyclobutane-1-carboxylate (233 mg, 0.95 mmol) in n-butanol (3 mL) was added potassium carbonate (173 mg, 1.26 mmol). The reaction mixture was heated at 130° C. in a microwave reactor for three hours, quenched with water (30 mL) and extracted with ethyl acetate (50 mL×2). The combined organic extracts were washed with brine (50 mL×2), dried over sodium sulfate, filtered and concentrated. The resulting residue was purified by flash chromatography (35-50% ethyl acetate in petroleum ether) to afford the title compound as a yellow solid (40 mg, 11%). MS: 589 m/z [M+H]⁺.

### D. Example 248. 3-Fluoro-3-(3-((5-(2-fluoro-5-((6-fluoro-4-methyl-1H-indol-5-yl)oxy)phenyl)-4H-1,2,4-triazol-3-yl)methyl)phenyl)cyclobutane-1-carboxylic acid

To a stirred solution of butyl 3-fluoro-3-(3-((5-(2-fluoro-5-((6-fluoro-4-methyl-1H-indol-5-yl)oxy)phenyl)-4H-1,2,4-triazol-3-yl)methyl)phenyl)cyclobutane-1-carboxylate (40 mg, 0.068 mmol) in a 2:1 mixture of THF and methanol (3 mL) was added lithium hydroxide (1M in water) (1.0 mL, 1.0 mmol). The reaction mixture was stirred at room temperature for two hours, concentrated below 25° C. to remove THF and methanol, cooled to 0° C., acidified with hydrochloric acid (1 M) to pH 4 and extracted with ethyl acetate (30 mL×2). The combined organic extracts were washed with brine (40 mL), dried over sodium sulfate, filtered and concentrated. The resulting residue was purified by flash chromatography (5% methanol in dichloromethane) to afford the title compound as a white solid (6.5 mg, 18%). MS: 533 m/z [M+H]⁺. ¹H NMR (400 MHz, CD₃OD) δ 7.52-7.17 (m, 7H), 7.12 (d, J=10.4 Hz, 1H), 7.06-6.92 (m, 1H), 6.53 (d, J=2.9 Hz, 1H), 4.17 (s, 2H), 2.96-2.66 (m, 5H), 2.40 (s, 3H) ppm.

### Example 249

### 1-(3-((5-(2-Fluoro-5-((6-fluoro-4-methyl-1H-indol-5-yl)oxy)phenyl)-4H-1,2,4-triazol-3-yl)methyl)phenyl)azetidine-3-carboxylic acid

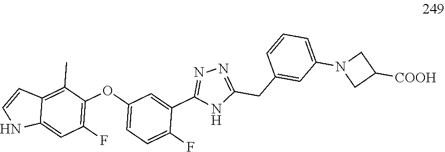 [SMILES: Cc1c(Oc2ccc(F)c(-c3nnc(Cc4cccc(N5CC(C(=O)O)C5)c4)[nH]3)c2)c(F)cc2[nH]ccc12]

### A. 1-(3-Iodophenyl)azetidin-3-ol

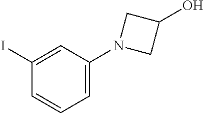 [SMILES: OC1CN(c2cccc(I)c2)C1]

To a stirred solution of 1,3-diiodobenzene (10.0 g, 30.3 mmol) in DMSO (100 mL) were added azetidin-3-ol hydrochloride (2.19 g, 20 mmol), CuI (750 mg, 3.94 mmol), L-proline (907 mg, 7.88 mmol), and cesium carbonate (19.74 g, 60.6 mmol). The reaction mixture was stirred at 90° C. for twelve hours under nitrogen, diluted with water (100 mL) and extracted with ethyl acetate (50 mL×3). The combined organic extracts were washed with brine, dried over sodium sulfate and concentrated. The resulting residue was purified by flash chromatography over silica (25-50% ethyl acetate in petroleum ether) to afford the title compound as a white solid (3.01 g, 57%). MS: 276 m/z [M+H]⁺.

### B. 1-(3-Iodophenyl)azetidin-3-yl methanesulfonate

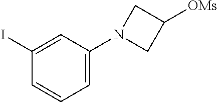 [SMILES: CS(=O)(=O)OC1CN(c2cccc(I)c2)C1]

To a stirred solution of 1-(3-iodophenyl)azetidin-3-ol (3.01 g, 10.94 mmol) and triethylamine (3.32 g, 32.82 mmol) in anhydrous dichloromethane (25 mL) was added methanesulfonyl chloride (1.32 g, 11.49 mmol) slowly at 0° C. The reaction mixture was stirred at room temperature for three hours, quenched with icy water and extracted with ethyl acetate (50 mL×3). The combined organic phase was washed with brine, dried over sodium sulfate and concentrated to afford the title compound as a yellow solid, which was used directly in the next step. MS: 354 m/z [M+H]⁺.

### C. 1-(3-Iodophenyl)azetidine-3-carbonitrile

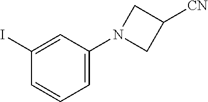 [SMILES: N#CC1CN(c2cccc(I)c2)C1]

A mixture of 1-(3-iodophenyl)azetidin-3-yl methanesulfonate and potassium cyanide (1.35 g, 20.56 mmol) in DMF (25 mL) and water (4 mL) was stirred at 70° C. for twelve hours, diluted with water (80 mL) and extracted with ethyl acetate (50 mL×3). The combined organic extracts were washed with brine, dried over sodium sulfate and concentrated. The residue was purified by flash chromatography over silica (petroleum ether/ethyl acetate, v/v, 8/1 to 4/1) to afford the title compound as a white solid (1.46 g, 47%, two steps). MS: 285 m/z [M+H]⁺.

### D. Methyl 1-(3-iodophenyl)azetidine-3-carboxylate

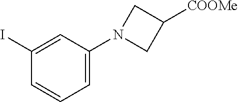 [SMILES: COC(=O)C1CN(c2cccc(I)c2)C1]

A mixture of 1-(3-iodophenyl)azetidine-3-carbonitrile (540 mg, 1.90 mmol) and potassium hydroxide (106 mg, 19 mmol) in 1,4-dioxane (10 mL) and water (1 mL) was stirred at 110° C. for twelve hours. The pH of the reaction mixture was adjusted to 4-5 with 1N HCl, concentrated to remove organic solvent and extracted with ethyl acetate (50 mL×6). The combined organic extracts were dried over sodium sulfate, filtered and concentrated. To a stirred solution of the resulting residue in DMF (4 mL) were added methanol (64 mg, 2.0 mmol), triethylamine (404 mg, 4.0 mmol) and HATU (570 mg, 1.5 mmol). The mixture was then stirred at room temperature for sixteen hours, diluted with water and extracted with ethyl acetate (30 mL×3). The combined organic phase was washed with brine, dried over sodium sulfate and concentrated. The residue was purified by flash chromatography over silica (petroleum ether/ethyl acetate, v/v, 8/1 to 4/1) to afford the title compound as a white solid (287 mg, 50%). MS: 318 m/z [M+H]⁺.

### E. Methyl 1-(3-(cyanomethyl)phenyl)azetidine-3-carboxylate

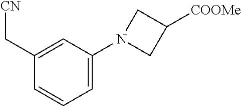 [SMILES: COC(=O)C1CN(c2cccc(CC#N)c2)C1]

The title compound was similarly prepared according to the procedure described for methyl 3-(3-(cyanomethyl)phenyl)-3-fluorocyclobutane-1-carboxylate but exchanging methyl 3-(3-bromophenyl)-3-fluorocyclobutane-1-carboxylate for methyl 1-(3-iodophenyl)azetidine-3-carboxylate as a yellow oil. MS: 231 m/z [M+H]⁺.

### F. Example 249. 1-(3-((5-(2-Fluoro-5-((6-fluoro-4-methyl-1H-indol-5-yl)oxy)phenyl)-4H-1,2,4-triazol-3-yl)methyl)phenyl)azetidine-3-carboxylic acid

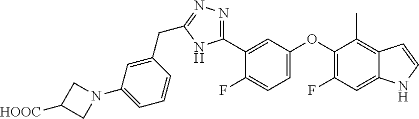 [SMILES: Cc1c(Oc2ccc(F)c(-c3nnc(Cc4cccc(N5CC(C(=O)O)C5)c4)[nH]3)c2)c(F)cc2[nH]ccc12]

Exchanging methyl 3-(3-(cyanomethyl)phenyl)-3-fluorocyclobutane-1-carboxylate for methyl 1-(3-(cyanomethyl)phenyl)azetidine-3-carboxylate, the two step reaction sequence described for steps C and D in Example 248 was used to prepare the title compound. LC-MS retention time: 1.18 minutes. MS: 516 m/z [M+H]⁺. ¹H NMR (400 MHz, CD₃OD) δ 7.36 (t, J=2.0 Hz, 1H), 7.28 (d, J=3.2 Hz, 1H), 7.18 (t, J=7.2 Hz, 1H), 7.14-7.10 (m, 2H), 7.00-6.99 (m, 1H), 6.63 (d, J=7.2 Hz, 1H), 6.53 (d, J=3.2 Hz, 1H), 6.43 (s, 1H), 6.38 (d, J=8.4 Hz, 1H), 4.05 (s, 2H), 4.00 (t, J=7.6 Hz, 2H), 3.91 (t, J=7.6 Hz, 2H), 3.45-3.44 (m, 1H), 2.40 (s, 3H) ppm.

### Example 250

### 3-(3-(1-(5-(5-((4,6-Difluoro-1H-indol-5-yl)oxy)-2-fluorophenyl)-4H-1,2,4-triazol-3-yl)-1-hydroxyethyl)phenyl)propanoic acid

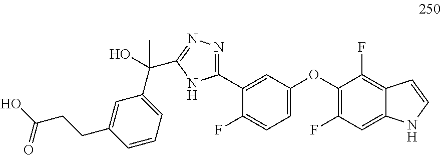 [SMILES: CC(O)(c1cccc(CCC(=O)O)c1)c1nnc(-c2cc(Oc3c(F)cc4[nH]ccc4c3F)ccc2F)[nH]1]

### A. 2-(3-Iodophenyl)-2-((trimethylsilyl)oxy)acetonitrile

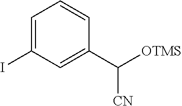

To a stirred solution of 3-iodobenzaldehyde (10.0 g, 43.1 mmol) in dichloromethane (100 mL) was added zinc iodide (6.9 g, 21.6 mmol) followed by the dropwise addition of trimethylsilylcyanide (13.4 mL, 107.8 mmol) at 0° C. under nitrogen atmosphere. The reaction mixture was stirred at room temperature overnight and filtered through a pad of Celite. The filtrate was washed with water (30 mL×3) and brine (30 mL), dried over sodium sulfate, filtered and concentrated to afford the title compound as a brown oil (12.5 g, crude), which was used in the next step without further purification. ¹H-NMR (400 MHz, CDCl₃) δ 7.83 (s, 1H), 7.75 (d, J=7.6 Hz, 1H), 7.46 (d, J=8.0 Hz, 1H), 7.20-7.16 (m, 1H), 5.45 (s, 1H), 0.27 (s, 9H) ppm.

### B. Methyl 2-hydroxy-2-(3-iodophenyl)acetate

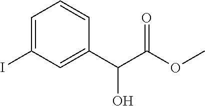

A mixture of 2-(3-iodophenyl)-2-((trimethylsilyl)oxy)acetonitrile (12.5 g, 48.4 mmol) in a solution of 3M hydrochloric acid in methanol (150 mL) was refluxed for two days and concentrated. The resulting residue was dissolved in ethyl acetate (150 mL), washed with brine (50 mL), dried over sodium sulfate, filtered and concentrated. The resulting crude product was purified by flash chromatography over silica (ethyl acetate/petroleum ether, v/v, 1/5) to afford the title compound as a yellow solid (4.9 g, 39% from 2 steps). ¹H-NMR (400 MHz, CDCl₃) δ 7.81 (s, 1H), 7.69 (d, J=8.0 Hz, 1H), 7.41 (d, J=8.0 Hz, 1H), 7.14-7.10 (m, 1H), 5.14 (d, J=4.8 Hz, 1H), 3.80 (s, 3H), 3.51 (d, J=5.2 Hz, 1H) ppm.

### C. 2-Hydroxy-2-(3-iodophenyl)acetohydrazide

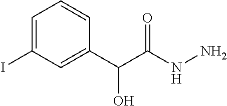 [SMILES: NNC(=O)C(O)c1cccc(I)c1]

To a stirred solution of methyl 2-hydroxy-2-(3-iodophenyl)acetate (4.9 g, 16.8 mmol) in methanol (30 mL) was added hydrazine hydrate (4.2 g, 84.0 mmol). The resulting mixture was refluxed for one hour and concentrated to afford the title compound as a yellow solid (4.4 g, 91%), which was used for next step without further purification. MS: 293 m/z [M+H]⁺.

### D. (5-(5-((4,6-Difluoro-1H-indol-5-yl)oxy)-2-fluorophenyl)-4H-1,2,4-triazol-3-yl)(3-iodophenyl)methanol

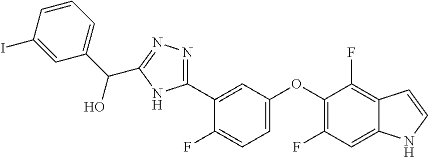 [SMILES: OC(c1cccc(I)c1)c1nnc(-c2cc(Oc3c(F)cc4[nH]ccc4c3F)ccc2F)[nH]1]

To a stirred solution of 5-((4,6-difluoro-1H-indol-5-yl)oxy)-2-fluorobenzimidamide (600 mg, 0.66 mmol, Intermediate 24-9) in ethanol (10 mL) was added a solution of triethylamine (10 mL) and 2-hydroxy-2-(3-iodophenyl)acetohydrazide (1.15 g, 3.94 mmol). The reaction mixture was stirred at 70° C. overnight and concentrated. The resulting residue was purified by flash chromatography over silica (dichloromethane/ethyl acetate, v/v, 5/1) to afford the title compound as a yellow solid (480 mg, 37% from 2 steps). MS: 563 m/z [M+H]⁺.

### E. (5-(5-((4,6-Difluoro-1H-indol-5-yl)oxy)-2-fluorophenyl)-4H-1,2,4-triazol-3-yl)(3-iodophenyl)methanone

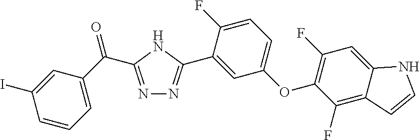 [SMILES: O=C(c1cccc(I)c1)c1nnc(-c2cc(Oc3c(F)cc4[nH]ccc4c3F)ccc2F)[nH]1]

To a stirred solution of (5-(5-((4,6-difluoro-1H-indol-5-yl)oxy)-2-fluorophenyl)-4H-1,2,4-triazol-3-yl)(3-iodophenyl)methanol (480 mg, 0.85 mmol) in THF (15 mL) was added manganese dioxide powder (743 mg, 8.54 mmol). The reaction mixture was stirred at room temperature overnight, filtered through Celite and the filtrate was concentrated to afford the title compound (440 mg, 92%), which was used for next step without further purification. MS: 561 m/z [M+H]⁺.

### F. Ethyl 3-(3-(5-(5-((4,6-difluoro-1H-indol-5-yl)oxy)-2-fluorophenyl)-4H-1,2,4-triazole-3-carbonyl)phenyl)propanoate

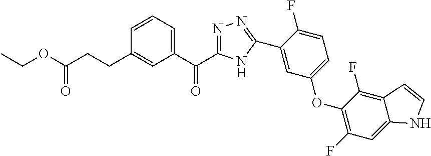 [SMILES: CCOC(=O)CCc1cccc(C(=O)c2nnc(-c3cc(Oc4c(F)cc5[nH]ccc5c4F)ccc3F)[nH]2)c1]

To a mixture of (5-(5-((4,6-difluoro-1H-indol-5-yl)oxy)-2-fluorophenyl)-4H-1,2,4-triazol-3-yl)(3-iodophenyl)methanone (440 mg, 0.79 mmol), tributylamine (292 mg, 1.58 mmol), tetrabutylammonium chloride (220 mg, 0.79 mmol) and 3,3-diethoxyprop-1-ene (308 mg, 2.37 mmol) in DMF (3 mL) was added palladium(II) acetate (18 mg, 0.08 mmol), the reaction mixture was stirred at 70° C. for two hours in a glove box and diluted with ethyl acetate (20 mL). The organic phase was washed with water (5 mL×2) and brine (5 mL), dried over sodium sulfate, filtered and concentrated. The resulting residue was purified by preparative TLC (developed with dichloromethane/ethyl acetate, v/v, 8/1) to afford the title compound as a yellow solid (140 mg, 33%). MS: 535 m/z [M+H]⁺.

### G. 3-(3-(5-(5-((4,6-Difluoro-1H-indol-5-yl)oxy)-2-fluorophenyl)-4H-1,2,4-triazole-3-carbonyl)phenyl)propanoic acid

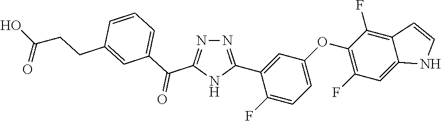 [SMILES: O=C(O)CCc1cccc(C(=O)c2nnc(-c3cc(Oc4c(F)cc5[nH]ccc5c4F)ccc3F)[nH]2)c1]

To a stirred solution of ethyl 3-(3-(5-(5-((4,6-difluoro-1H-indol-5-yl)oxy)-2-fluorophenyl)-4H-1,2,4-triazole-3-carbonyl)phenyl)propanoate (140 mg, 0.26 mmol) in THF (2 mL) was added a solution of lithium hydroxide (24 mg, 1.04 mmol) in water (1 mL). The resulting mixture was stirred at 60° C. for one hour, concentrated to remove THF and acidified with 1 N hydrochloric acid to pH˜2. The precipitate was collected by filtration and dried to afford the title compound as a white solid (130 mg, 93%), which was used directly for next step. MS: 507 m/z [M+H]⁺.

### H. Example 250. 3-(3-(1-(5-(5-((4,6-Difluoro-1H-indol-5-yl)oxy)-2-fluorophenyl)-4H-1,2,4-triazol-3-yl)-1-hydroxyethyl)phenyl)propanoic acid

To a stirred solution of 3-(3-(5-(5-((4,6-difluoro-1H-indol-5-yl)oxy)-2-fluorophenyl)-4H-1,2,4-triazole-3-carbonyl)phenyl)propanoic acid (100 mg, 0.19 mmol) in THF (1 mL) was added a solution of methylmagnesium bromide in THF (3M, 0.2 mL, 0.6 mmol) at room temperature under nitrogen. The reaction mixture was stirred for thirty minutes, quenched with water (10 mL) and extracted with ethyl acetate (10 mL×5). The combined organic phase was washed with brine (10 mL), dried over sodium sulfate, filtered and concentrated. The resulting residue was purified by preparative HPLC to afford the title compound as a white solid (15.2 mg, 15%). LC-MS retention time 1.14 minutes. MS: 523 m/z [M+H]⁺. ¹H NMR (400 MHz, CD₃OD) δ 7.50 (m, 1H), 7.44 (s, 1H), 7.34-7.31 (m, 2H), 7.23 (t, J=7.6 Hz, 2H), 7.13 (t, J=6.8 Hz, 2H), 7.06-7.01 (m, 1H), 6.56 (d, J=3.2 Hz, 1H), 2.90 (t, J=7.6 Hz, 2H), 2.59 (t, J=7.6 Hz, 2H), 1.97 (s, 3H) ppm.

The following example was prepared utilizing the similar procedures as described for Example 250.

| | Ex. | | | LCMS | MS m/z |
|---|---|---|---|---|---|
| Structure | No. | Name | ¹H NMR | Rₜ (min) | [M + H]⁺ |
| 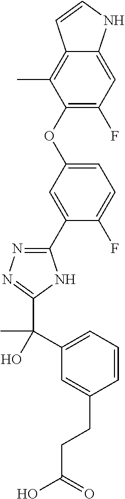 [SMILES: Cc1c(Oc2ccc(F)c(-c3nnc(C(C)(O)c4cccc(CCC(=O)O)c4)[nH]3)c2)c(F)cc2[nH]ccc12] | 251 | 3-(3-(1-(5-(2-fluoro- 5-((6-fluoro-4- methyl-1H-indol-5- yl)oxy)phenyl)-4H- 1,2,4-triazol-3-yl)-1- hydroxyethyl)phenyl) propanoic acid | (400 MHz, CD₃OD) δ 7.43-7.40 (m, 2H), 7.34-7.32 (m, 1H), 7.28 (d, J = 3.2 Hz, 1H), 7.25-7.10 (m, 4H), 6.93 (s, 1H), 6.52 (d, J = 3.6 Hz, 1H), 2.90 (t, J = 8.0 Hz, 2H), 2.56 (t, J = 8.4 Hz, 2H), 2.39 (s, 3H), 1.96 (s, 3H) ppm. | 1.17 | 519 |

### Example 252

### Enantiomer 1 of 3-(3-(1-(5-(5-((4,6-difluoro-1H-indol-5-yl)oxy)-2-fluorophenyl)-4H-1,2,4-triazol-3-yl)ethyl)-2-fluorophenyl)propanoic acid (252A)

### And

### Enantiomer 2 of 3-(3-(1-(5-(5-((4,6-difluoro-1H-indol-5-yl)oxy)-2-fluorophenyl)-4H-1,2,4-triazol-3-yl)ethyl)-2-fluorophenyl)propanoic acid (252B)

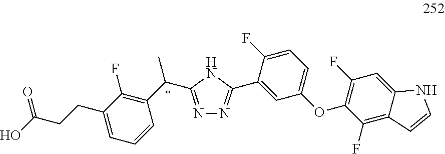 [SMILES: CC(c1nnc(-c2cc(Oc3c(F)cc4[nH]ccc4c3F)ccc2F)[nH]1)c1cccc(CCC(=O)O)c1F]

### A. 5-(3-(5-(1-(3-Bromo-2-fluorophenyl)ethyl)-4H-1,2,4-triazol-3-yl)-4-fluorophenoxy)-4,6-difluoro-1H-indole

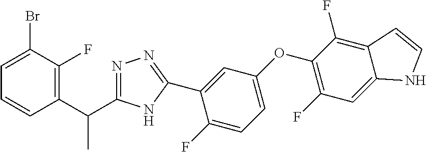 [SMILES: CC(c1nnc(-c2cc(Oc3c(F)cc4[nH]ccc4c3F)ccc2F)[nH]1)c1cccc(Br)c1F]

Exchanging 2-hydroxy-2-(3-iodophenyl)acetohydrazide for 2-(3-bromo-2-fluoro-phenyl)propanehydrazide (2.40 g, 90% purity, 8.25 mmol), the reaction procedure described for Step D of Example 250 was used to prepare the title compound as a light yellow solid (1.0 g, 44.2%). MS: 531, 533 m/z [M+H]⁺.

### B. Ethyl 3-(3-(1-(5-(5-((4,6-difluoro-1H-indol-5-yl)oxy)-2-fluorophenyl)-4H-1,2,4-triazol-3-yl)ethyl)-2-fluorophenyl)propanoate

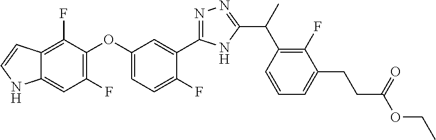 [SMILES: CCOC(=O)CCc1cccc(C(C)c2nnc(-c3cc(Oc4c(F)cc5[nH]ccc5c4F)ccc3F)[nH]2)c1F]

Exchanging 3-(3-bromophenyl)tetrahydrofuran-3-carboxylic acid for 5-(3-(5-(1-(3-bromo-2-fluorophenyl)ethyl)-4H-1,2,4-triazol-3-yl)-4-fluorophenoxy)-4,6-difluoro-1H-indole (600 mg, 1.10 mmol), the two step reaction sequence described for Intermediate 21-1B to 21-1D was used to prepare the title compound as a light yellow solid (360 mg, 57%). MS: 553 m/z [M+H]⁺.

### C. Chiral Separation of Enantiomers

Racemic ethyl 3-(3-(1-(5-(5-((4,6-difluoro-1H-indol-5-yl)oxy)-2-fluorophenyl)-4H-1,2,4-triazol-3-yl)ethyl)-2-fluorophenyl)propanoate (360 mg) was separated by SFC using a (R,R)-WHELK-O1 (20×250 mm, 10 micron) column, eluting with 50% carbon dioxide, 50% methanol (0.2% saturated ammonia in methanol), at a back pressure of 100 bar, a flow rate of 80 g/min and column temperature of 35° C.

Peak 1: Pure enantiomer 1 of ethyl 3-(3-(1-(5-(5-((4,6-difluoro-1H-indol-5-yl)oxy)-2-fluorophenyl)-4H-1,2,4-triazol-3-yl)ethyl)-2-fluorophenyl)propanoate (148 mg, 100% ee), MS: 553 m/z [M+H]⁺. ¹H NMR (400 MHz, CD₃OD) δ 7.50 (s, 1H), 7.31 (d, J=2.8 Hz, 1H), 7.24-7.03 (m, 6H), 6.56 (d, J=2.8 Hz, 1H), 4.66-4.63 (m, 1H), 4.08 (q, J=7.2 Hz, 2H), 2.96 (t, J=7.6 Hz, 2H), 2.62 (t, J=7.6 Hz, 2H), 1.70-1.65 (m, 3H), 1.19 (t, J=7.6 Hz, 3H) ppm.

Peak 2: Pure enantiomer 2 of ethyl 3-(3-(1-(5-(5-((4,6-difluoro-1H-indol-5-yl)oxy)-2-fluorophenyl)-4H-1,2,4-triazol-3-yl)ethyl)-2-fluorophenyl)propanoate (155 mg, 100% ee). MS: 553 m/z [M+H]⁺.

### D. Example 252A Enantiomer 1 of 3-(3-(1-(5-(5-((4,6-difluoro-1H-indol-5-yl)oxy)-2-fluorophenyl)-4H-1,2,4-triazol-3-yl)ethyl)-2-fluorophenyl)propanoic acid

Exchanging ethyl 3-(3-(5-(5-((4,6-difluoro-1H-indol-5-yl)oxy)-2-fluorophenyl)-4H-1,2,4-triazole-3-carbonyl)phenyl)propanoate for enantiomer 1 of ethyl 3-(3-(1-(5-(5-((4,6-difluoro-1H-indol-5-yl)oxy)-2-fluorophenyl)-4H-1,2,4-triazol-3-yl)ethyl)-2-fluorophenyl)propanoate, the reaction procedure described for Step G of Example 250 was used to prepare the title compound as a white solid (120 mg, 90%). ¹H NMR (400 MHz, CD₃OD) δ 7.50 (s, 1H), 7.31 (d, J=2.8 Hz, 1H), 7.23-7.04 (m, 6H), 6.56 (d, J=2.8 Hz, 1H), 4.66 (q, J=6.0 Hz, 1H), 2.95 (t, J=7.6 Hz, 2H), 2.60 (t, J=7.6 Hz, 2H), 1.69 (d, J=6.0 Hz, 3H) ppm. MS: 525 m/z [M+H]⁺.

### E. Example 252B Enantiomer 2 of 3-(3-(1-(5-(5-((4,6-difluoro-1H-indol-5-yl)oxy)-2-fluorophenyl)-4H-1,2,4-triazol-3-yl)ethyl)-2-fluorophenyl)propanoic acid

Exchanging ethyl 3-(3-(5-(5-((4,6-difluoro-1H-indol-5-yl)oxy)-2-fluorophenyl)-4H-1,2,4-triazole-3-carbonyl)phenyl)propanoate for enantiomer 2 of ethyl 3-(3-(1-(5-(5-((4,6-difluoro-1H-indol-5-yl)oxy)-2-fluorophenyl)-4H-1,2,4-triazol-3-yl)ethyl)-2-fluorophenyl)propanoate, the reaction procedure described for Step G of Example 250 was used to prepare the title compound as a white solid (120 mg, 90%). ¹H NMR (400 MHz, CD₃OD) δ 7.51 (s, 1H), 7.31 (d, J=2.8 Hz, 1H), 7.23-7.03 (m, 6H), 6.56 (d, J=2.8 Hz, 1H), 4.66 (q, J=6.0 Hz, 1H), 2.95 (t, J=7.6 Hz, 2H), 2.60 (t, J=7.6 Hz, 2H), 1.69 (d, J=6.0 Hz, 3H) ppm. MS: 525 m/z [M+H]⁺.

### Examples 253 and 254

### 7-(4-((5-(3-((1H-Indol-5-yl)oxy)phenyl)-4H-1,2,4-triazol-3-yl)methyl)phenyl)hept-6-ynoic acid

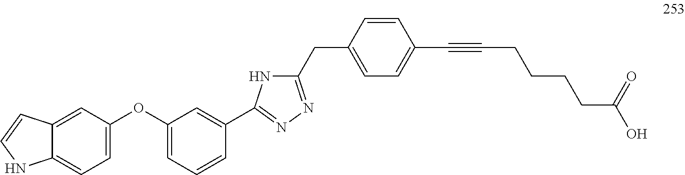 [SMILES: O=C(O)CCCCC#Cc1ccc(Cc2nnc(-c3cccc(Oc4ccc5[nH]ccc5c4)c3)[nH]2)cc1]

### And

### 7-(4-((5-(3-((1H-Indol-5-yl)oxy)phenyl)-4H-1,2,4-triazol-3-yl)methyl)phenyl)heptanoic acid

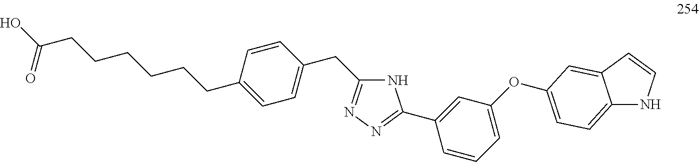 [SMILES: O=C(O)CCCCCCc1ccc(Cc2nnc(-c3cccc(Oc4ccc5[nH]ccc5c4)c3)[nH]2)cc1]

### A. tert-Butyl 5-(3-(ethoxycarbonyl)phenoxy)-1H-indole-1-carboxylate

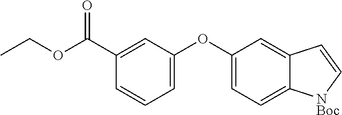 [SMILES: CCOC(=O)c1cccc(Oc2ccc3c(ccn3C(=O)OC(C)(C)C)c2)c1]

To a stirred solution of tert-butyl 5-hydroxy-1H-indole-1-carboxylate (5.1 g 21.9 mmol) and (3-(ethoxycarbonyl)phenyl)boronic acid (6.36 g, 32.8 mmol) in dichloromethane (150 mL) were added copper(II) acetate (5.04 g, 27.8 mmol), triethylamine (11.8 g, 116.5 mmol) and 4 Å molecular sieves (6 gm). The reaction mixture was stirred at room temperature for five hours and filtered through a pad of Celite. The filtrate was diluted with ethyl acetate (200 mL), washed with water and brine, dried over sodium sulfate and concentrated to dryness. The resulting residue was absorbed onto Celite and purified by silica gel chromatography (0-20% ethyl acetate in heptane) to afford the title compound as a sticky oil.

### B. 3-((1H-Indol-5-yl)oxy)benzohydrazide

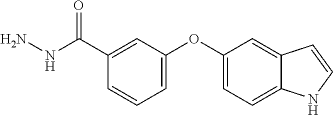 [SMILES: NNC(=O)c1cccc(Oc2ccc3[nH]ccc3c2)c1]

(This reaction was carried out in three 20 mL microwave reaction vials and then combined to workup after heating in a microwave reactor). A solution of tert-butyl 5-(3-(methoxycarbonyl)phenoxy)-1H-indole-1-carboxylate (7.3 g, 19.87 mmol) and hydrazine hydrate (6.24 mL, 199 mmol) in methanol (13×3 mL) was heated at 135° C. in a microwave reactor for one hour. The solvent was evaporated and the residue was chromatographed over silica (0-10% methanol in dichloromethane containing 0.5% ammonia) to afford the title compound as a colorless oil (5 g, 94%) which solidified to give a white solid upon standing. MS: 268 m/z [M+H]⁺.

### C. 5-(3-(5-(4-Iodobenzyl)-4H-1,2,4-triazol-3-yl)phenoxy)-1H-indole

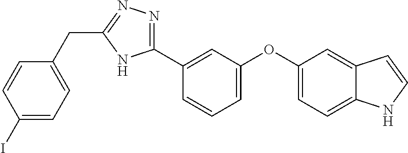 [SMILES: Ic1ccc(Cc2nnc(-c3cccc(Oc4ccc5[nH]ccc5c4)c3)[nH]2)cc1]

A mixture of 3-((1H-indol-5-yl)oxy)benzohydrazide (1.9 μg, 7.1 mmol), 2-(4-iodophenyl)acetonitrile (4.32 g, 17.8 mmol) and potassium carbonate (1.28 g, 9.24 mmol) in n-butanol (16 mL) was heated in a 20 mL microwave vial at 165° C. for 1.5 hours. The solvent was evaporated and the residue chromatographed (0-100% ethyl acetate in heptane) to afford the title compound as a yellow solid (2.5 g, 71%). MS: 493 m/z [M+H]⁺.

### D. Methyl 7-(4-((5-(3-((1H-indol-5-yl)oxy)phenyl)-4H-1,2,4-triazol-3-yl)methyl)phenyl)hept-6-ynoate

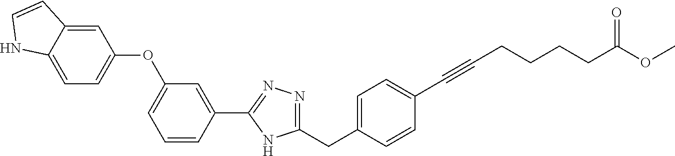 [SMILES: COC(=O)CCCCC#Cc1ccc(Cc2nnc(-c3cccc(Oc4ccc5[nH]ccc5c4)c3)[nH]2)cc1]

A solution of 5-(3-(5-(4-iodobenzyl)-4H-1,2,4-triazol-3-yl)phenoxy)-1H-indole (0.35 g, 710 μmol) and methyl hept-6-ynoate (149 mg, 1.07 mmol) in DMF (0.5 mL) and triethylamine (1 mL, 7.2 mmol) was degassed for five minutes and then bis(triphenylphosphine)palladium(II) chloride (50 mg, 71 μmol) and copper(I) iodide (13 mg, 71 μmol) were added. The reaction mixture was heated at 70° C. for one hour, the solvent was evaporated and the residue was purified by flash chromatography over silica (0-80% ethyl acetate in heptane) twice to afford the title compound as a yellow solid (0.24 g, 67%). MS: 505[M+H]⁺. ¹H NMR (400 MHz, CD₃OD) δ 7.61 (d, J=7.4 Hz, 1H), 7.54 (s, 1H), 7.43-7.16 (m, 8H), 6.99 (d, J=6.4 Hz, 1H), 6.86 (dd, J=8.7, 2.3 Hz, 1H), 6.42 (d, J=3.1 Hz, 1H), 4.09 (s, 2H), 3.65 (s, 3H), 2.41-2.37 (m, 4H), 1.76 (p, J=7.4 Hz, 2H), 1.65-1.53 (m, 2H) ppm.

### E. Example 253. 7-(4-((5-(3-((1H-Indol-5-yl)oxy)phenyl)-4H-1,2,4-triazol-3-yl)methyl)phenyl)hept-6-ynoic acid

A stirred solution of methyl 7-(4-((5-(3-((1H-indol-5-yl)oxy)phenyl)-4H-1,2,4-triazol-3-yl)methyl)phenyl)hept-6-ynoate (30 mg, 59 μmol) and lithium hydroxide (2.9 mg, 120 μmol) in methanol (1 mL) and water (0.5 mL) was stirred at room temperature overnight. Methanol was evaporated and the residue was acidified to pH˜2 with 1N HCl. The white solid was collected by filtration and dried to afford the title compound as a white solid (28 mg, 95%). MS: 491 m/z [M+H]⁺. ¹H NMR (400 MHz, CD₃OD) δ 7.60 (d, J=7.7 Hz, 1H), 7.54-7.48 (m, 1H), 7.48-7.37 (m, 2H), 7.37-7.20 (m, 6H), 7.08 (dd, J=8.2, 2.4 Hz, 1H), 6.87 (dd, J=8.7, 2.3 Hz, 1H), 6.43 (d, J=2.4 Hz, 1H), 4.19 (s, 2H), 2.42-2.37 (m, 4H), 1.78-1/74 (m, 2H), 1.64-1.58 (m, 2H) ppm.

### F. Methyl 7-(4-((5-(3-((1H-indol-5-yl)oxy)phenyl)-4H-1,2,4-triazol-3-yl)methyl)phenyl)heptanoate

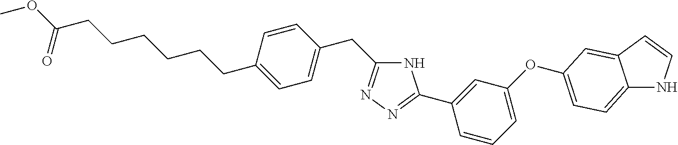 [SMILES: COC(=O)CCCCCCc1ccc(Cc2nnc(-c3cccc(Oc4ccc5[nH]ccc5c4)c3)[nH]2)cc1]

To a stirred solution of methyl 7-(4-((5-(3-((1H-indol-5-yl)oxy)phenyl)-4H-1,2,4-triazol-3-yl)methyl)phenyl)hept-6-ynoate (0.2 g, 396 μmol) in ethyl acetate (15 mL) was added Pd—C (168 mg, 10% on carbon) and the reaction mixture was stirred under hydrogen balloon for four hours. The mixture was filtered through a pad of Celite, the solvent was evaporated and the residue was chromatographed (0-100% ethyl acetate in heptane) to afford the title compound as a white solid (0.15 g, 74%). MS: 509 m/z [M+H]⁺. ¹H NMR (400 MHz, CD₃OD) δ 7.61 (d, J=7.7 Hz, 1H), 7.54 (d, J=2.0 Hz, 1H), 7.42-7.31 (m, 2H), 7.25 (dd, J=10.9, 2.6 Hz, 2H), 7.19-7.06 (m, 4H), 6.98 (dd, J=8.2, 1.8 Hz, 1H), 6.86 (dd, J=8.7, 2.2 Hz, 1H), 6.41 (d, J=3.0 Hz, 1H), 4.06 (s, 2H), 3.62 (s, 3H), 2.54 (t, J=7.5 Hz, 2H), 2.27 (t, J=7.4 Hz, 2H), 1.60-1.51 (m, 4H), 1.33-1.26 (m, 4H) ppm.

### G. Example 254. 7-(4-((5-(3-((1H-Indol-5-yl)oxy)phenyl)-4H-1,2,4-triazol-3-yl)methyl)phenyl)heptanoic acid

To a stirred solution of methyl 7-(4-((5-(3-((1H-indol-5-yl)oxy)phenyl)-4H-1,2,4-triazol-3-yl)methyl)phenyl)heptanoate (80 mg, 157 μmol) in methanol (2 mL) and water (1 mL) was added lithium hydroxide (3.8 mg, 157 μmol) and the reaction mixture was stirred at room temperature overnight. Methanol was evaporated and the residue was acidified with 1N hydrochloric acid to pH˜2. The white solid was collected by filtration and dried to afford the title compound (70 mg, 90%). MS m/z: 495 m/z [M+H]⁺. ¹H NMR (400 MHz, CD₃OD) δ 7.65-7.57 (m, 1H), 7.55-7.49 (m, 1H), 7.42 (t, J=9.0 Hz, 2H), 7.31-7.11 (m, 6H), 7.06 (d, J=7.9 Hz, 1H), 6.87 (dd, J=8.7, 2.3 Hz, 1H), 6.45-6.39 (m, 1H), 4.16 (s, 2H), 2.62-2.54 (m, 2H), 2.25 (t, J=7.4 Hz, 2H), 1.59-1.54 (m, 4H), 1.37-1.31 (m, 4H) ppm.

The following examples were prepared utilizing similar procedures as described for Example 254.

| | Ex. | Name | | LCMS | MS m/z |
|---|---|---|---|---|---|
| Structure | No. | | ¹H NMR | Rₜ (min) | [M + H]⁺ |
| 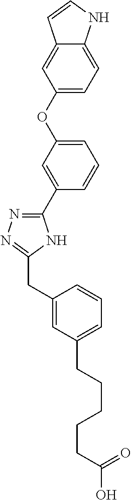 [SMILES: O=C(O)CCCCCc1cccc(Cc2nnc(-c3cccc(Oc4ccc5[nH]ccc5c4)c3)[nH]2)c1] | 255 | 6-(3-((5-(3-((1H- indol-5- yl)oxy)phenyl)-4H- 1,2,4-triazol-3- yl)methyl)phenyl) hexanoic acid | (400 MHz, CD₃OD) δ 7.61 (dd, J = 7.7, 1.0 Hz, 1H), 7.55-7.49 (m, 1H), 7.47-7.37 (m, 2H), 7.31-7.18 (m, 3H), 7.15-7.03 (m, 4H), 6.87 (dd, J = 8.7, 2.3 Hz, 1H), 6.42 (dd, J = 3.1, 0.7 Hz, 1H), 4.17 (s, 2H), 2.64-2.55 (m, 2H), 2.28-2.24 (m, 2H), 1.68-1.55 (m, 4H), 1.41-1.28 (m, 2H) ppm. | 1.30 | 481 |
| 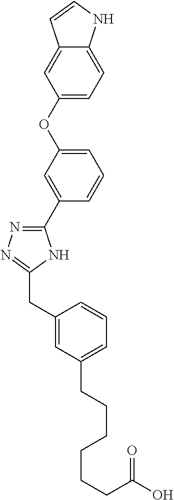 [SMILES: O=C(O)CCCCCCc1cccc(Cc2nnc(-c3cccc(Oc4ccc5[nH]ccc5c4)c3)[nH]2)c1] | 256 | 7-(3-((5-(3-((1H- indol-5- yl)oxy)phenyl)-4H- 1,2,4-triazol-3- yl)methyl)phenyl) heptanoic acid | (400 MHz, CD₃OD) δ 7.61 (d, J = 7.8 Hz, 1H), 7.55-7.49 (m, 2H), 7.44-7.40 (m, 2H), 7.31-7.18 (m, 3H), 7.14-7.01 (m, 4H), 6.87 (dd, J = 8.7, 2.3 Hz, 1H), 6.42 (dd, J = 3.1, 0.7 Hz, 1H), 4.16 (s, 2H), 2.63- 2.54 (m, 2H), 2.24 (t, J = 7.4 Hz, 2H), 1.59-1.54 (m, 4H), 1.36-1.30 (m, 4H) ppm. | 1.35 | 495 |
| 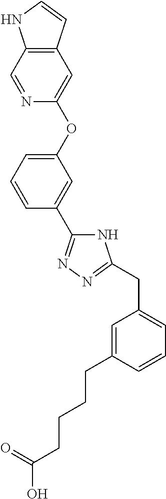 [SMILES: O=C(O)CCCCc1cccc(Cc2nnc(-c3cccc(Oc4cc5cc[nH]c5cn4)c3)[nH]2)c1] | 257 | 5-(3-((5-(3-((1H- pyrrolo[2,3- c]pyridin-5- yl)oxy)phenyl)-4H- 1,2,4-triazol-3- yl)methyl)phenyl) pentanoic acid | | 0.91 | 468 |
| 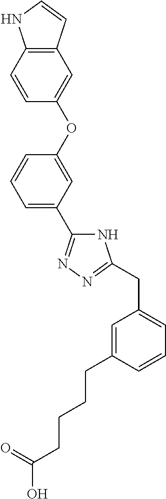 [SMILES: O=C(O)CCCCc1cccc(Cc2nnc(-c3cccc(Oc4ccc5[nH]ccc5c4)c3)[nH]2)c1] | 258 | 5-(3-((5-(3-((1H- indol-5- yl)oxy)phenyl)-4H- 1,2,4-triazol-3- yl)methyl)phenyl) pentanoic acid | (400 MHz, CD₃OD) δ 7.65-7.57 (m, 1H), 7.55-7.49 (m, 1H), 7.43-7.37 (m, 2H), 7.31-7.18 (m, 3H), 7.16-7.01 (m, 4H), 6.87 (dd, J = 8.7, 2.3 Hz, 1H), 6.42 (dd, J = 3.1, 0.8 Hz, 1H), 4.16 (s, 2H), 2.61 (t, J = 7.1 Hz, 2H), 2.29 (t, J = 6.9 Hz, 2H), 1.67-1.57 (m, 4H) ppm. | 1.26 | 467 |

### Example 259

### 4-(3-((5-(3-((1H-Indol-5-yl)oxy)phenyl)-4H-1,2,4-triazol-3-yl)methyl)phenyl)butanoic acid

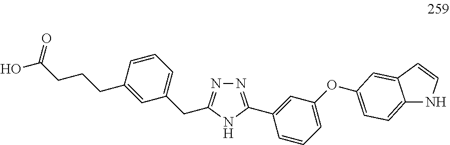 [SMILES: O=C(O)CCCc1cccc(Cc2nnc(-c3cccc(Oc4ccc5[nH]ccc5c4)c3)[nH]2)c1]

### A. 5-(3-(5-(3-Iodobenzyl)-4H-1,2,4-triazol-3-yl)phenoxy)-1H-indole

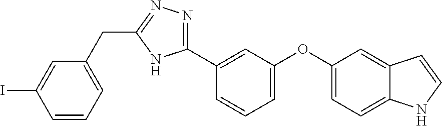 [SMILES: Ic1cccc(Cc2nnc(-c3cccc(Oc4ccc5[nH]ccc5c4)c3)[nH]2)c1]

A mixture of 3-((1H-indol-5-yl)oxy)benzohydrazide (1.9 g, 7.11 mmol, Example 253, Step B), 2-(3-iodophenyl)acetonitrile (4.32 g, 17.8 mmol) and potassium carbonate (1.18 g, 8.53 mmol) in n-butanol (15 mL) was heated at 165° C. for 1.5 hours. The solvent was evaporated and the residue was chromatographed over silica gel (0-100% ethyl acetate in heptane) to afford the title compound as a yellow solid (2.6 g, 74%). MS: 493 m/z [M+H]⁺.

### B. Methyl 4-(3-((5-(3-((1H-indol-5-yl)oxy)phenyl)-4H-1,2,4-triazol-3-yl)methyl)phenyl)butanoate

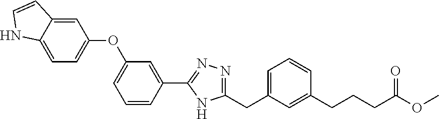 [SMILES: COC(=O)CCCc1cccc(Cc2nnc(-c3cccc(Oc4ccc5[nH]ccc5c4)c3)[nH]2)c1]

To a mixture of 5-(3-(5-(3-iodobenzyl)-4H-1,2,4-triazol-3-yl)phenoxy)-1H-indole (150 mg, 304.68 μmol), dicyclohexyl(2′,6′-dimethoxy-[1,1′-biphenyl]-2-yl)phosphine (125.08 mg, 304.68 μmol) and palladium(II) acetate (68.40 mg, 304.68 μmol) in a sealed microwave vial (2 mL) was added (4-ethoxy-4-oxobutyl)zinc(II) bromide (1.22 mL, 609.37 μmol) at room temperature under a flow of nitrogen and stirred for 30 minutes. The reaction mixture was diluted with ethyl acetate (50 mL), washed with concentratedammonium hydroxide solution, water and brine, dried over sodium sulfate, filtered and concentrated. The resulting residue was chromatographed (0-100% ethyl acetate in heptane) to afford the title compound as a white solid (100 mg, 68%). ¹H NMR (400 MHz, CD₃OD) δ 7.63-7.61 (m, 1H), 7.55-7.54 (m, 1H), 7.42-7.30 (m, 2H), 7.30-7.16 (m, 3H), 7.13-6.94 (m, 4H), 6.87-6.85 (m, 1H), 6.41 (d, J=3.1 Hz, 1H), 4.09-4.02 (m, 4H), 2.59 (t, J=7.6 Hz, 2H), 2.26 (t, J=7.6 Hz, 2H), 1.93-1.83 (m, 2H), 1.19 (t, J=7.1 Hz, 3H) ppm.

### C. Example 259. 4-(3-((5-(3-((1H-Indol-5-yl)oxy)phenyl)-4H-1,2,4-triazol-3-yl)methyl)phenyl)butanoic acid

To a stirred solution of ethyl 4-(3-((5-(3-((1H-indol-5-yl)oxy)phenyl)-4H-1,2,4-triazol-3-yl)methyl)phenyl)butanoate (80 mg, 166 μmol) in methanol (2 mL) and water (1 mL) was added lithium hydroxide (12 mg, 499 μmol) and the mixture was stirred overnight. Methanol was evaporated and the residue was acidified with 1N hydrochloric acid to pH˜2. The white precipitate was collected by filtration and dried to afford the title compound as a white solid (64 mg, 85%). LC-MS: Retention time 1.20 minutes. MS: 453 m/z [M+H]⁺. ¹H NMR (400 MHz, CD₃OD) δ 7.61 (dt, J=7.7, 1.3 Hz, 1H), 7.52 (dd, J=2.5, 1.6 Hz, 1H), 7.47-7.37 (m, 2H), 7.31-7.21 (m, 3H), 7.18-7.03 (m, 4H), 6.86 (dd, J=8.7, 2.3 Hz, 1H), 6.42 (dd, J=3.1, 1.0 Hz, 1H), 4.18 (s, 2H), 2.63 (dd, J=8.5, 6.8 Hz, 2H), 2.28 (t, J=7.4 Hz, 2H), 1.94-1.81 (m, 2H) ppm.

### Example 260

### 5-(3-(5-(3-(4-(1H-Tetrazol-5-yl)butyl)benzyl)-4H-1,2,4-triazol-3-yl)phenoxy)-1H-indole

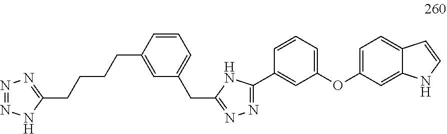 [SMILES: c1cc(CCCCc2nnn[nH]2)cc(Cc2nnc(-c3cccc(Oc4ccc5cc[nH]c5c4)c3)[nH]2)c1]

### A. 5-(3-((5-(3-((1H-Indol-5-yl)oxy)phenyl)-4H-1,2,4-triazol-3-yl)methyl)phenyl)pentanenitrile

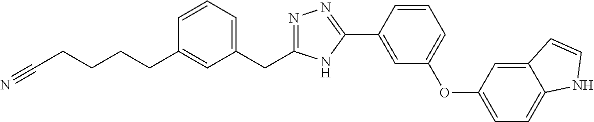 [SMILES: N#CCCCCc1cccc(Cc2nnc(-c3cccc(Oc4ccc5[nH]ccc5c4)c3)[nH]2)c1]

To a mixture of 5-(3-(5-(3-iodobenzyl)-4H-1,2,4-triazol-3-yl)phenoxy)-1H-indole (200 mg, 406 μmol, product of Step A of Example 259), dicyclohexyl(2′,6′-dimethoxy-[1,1′-biphenyl]-2-yl)phosphine (10 mg, 24 μmol) and palladium(II) acetate (2.7 mg, 12 μmol) in a sealed microwave vial (2 mL) was added (4-cyanobutyl)zinc(II) bromide (1 mL, 528 μmol) at room temperature under a nitrogen flow and heated at 50° C. for thirty minutes. The reaction mixture was diluted with ethyl acetate (60 mL), washed with concentrated aqueous ammonia, water and brine, dried over sodium sulfate, filtered and concentrated. The resulting residue chromatographed over silica (0-100% ethyl acetate in heptane) to afford the title compound as a white solid (100 mg, 55%). ¹H NMR (400 MHz, CD₃OD) δ 7.61 (dt, J=7.8, 1.2 Hz, 1H), 7.54 (dd, J=2.5, 1.5 Hz, 1H), 7.42-7.31 (m, 2H), 7.29-7.16 (m, 3H), 7.13-6.94 (m, 4H), 6.86 (dd, J=8.7, 2.3 Hz, 1H), 6.41 (dd, J=3.2, 0.9 Hz, 1H), 4.08 (s, 2H), 2.59 (t, J=7.5 Hz, 2H), 2.38 (t, J=7.0 Hz, 2H), 1.75-1.52 (m, 4H) ppm.

### B. Example 260. 5-(3-(5-(3-(4-(1H-Tetrazol-5-yl)butyl)benzyl)-4H-1,2,4-triazol-3-yl)phenoxy)-1H-indole

Triethylamine hydrochloride (185 mg, 1.34 mmol) and sodium azide (87 mg, 1.34 mmol) were added to a solution of 5-(3-((5-(3-((1H-indol-5-yl)oxy)phenyl)-4H-1,2,4-triazol-3-yl)methyl)phenyl)pentanenitrile (60 mg, 134 μmol) in DMF (1.5 mL) and the reaction mixture was heated at 130° C. in a microwave reactor for two hours. The solvent was evaporated and the residue purified by column chromatography on silica gel (0-100% ethyl acetate in hexane) to afford the title compound as a white solid (36 mg, 55%). LC-MS retention time 1.19 minutes. MS: 491 m/z [M+H]⁺. ¹H NMR (400 MHz, CD₃OD) δ 7.62 (dt, J=7.8, 1.2 Hz, 1H), 7.54 (dd, J=2.5, 1.5 Hz, 1H), 7.43-7.32 (m, 2H), 7.29-6.94 (m, 7H), 6.86 (dd, J=8.7, 2.3 Hz, 1H), 6.41 (dd, J=3.1, 0.9 Hz, 1H), 4.08 (s, 2H), 2.86 (t, J=7.5 Hz, 2H), 2.60 (t, J=7.6 Hz, 2H), 1.81-1.68 (m, 2H), 1.68-1.56 (m, 2H) ppm.

### Example 261

### 2-((3-((5-(3-((4,6-Difluoro-1H-indol-5-yl)oxy)phenyl)-4H-1,2,4-triazol-3-yl)methyl)phenyl)thio)acetic acid

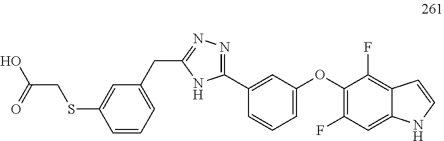 [SMILES: O=C(O)CSc1cccc(Cc2nnc(-c3cccc(Oc4c(F)cc5[nH]ccc5c4F)c3)[nH]2)c1]

### A. 4,6-Difluoro-5-(3-(5-(3-iodobenzyl)-4H-1,2,4-triazol-3-yl)phenoxy)-1H-indole

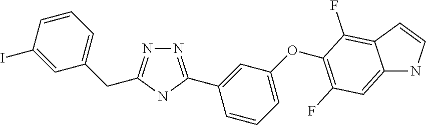 [SMILES: Fc1cc2[nH]ccc2c(F)c1Oc1cccc(-c2nnc(Cc3cccc(I)c3)[nH]2)c1]

To a solution of 3-((4,6-difluoro-1H-indol-5-yl)oxy)benzonitrile (Intermediate 6-14, 700 mg, 2.59 mmol) in n-BuOH (10 mL) was added 2-(3-iodophenyl)acetohydrazide (Intermediate 25, 2.86 g, 10.4 mmol) and potassium carbonate (357 mg, 5.18 mmol). The resulting mixture was irradiated at 160° C. for four hours in a Biotage microwave synthesizer. The reaction mixture was diluted with ethyl acetate (30 mL) and washed with water (10 mL×2), brine (10 mL), dried over anhydrous sodium sulfate, filtered and concentrated. The residue was purified by flash chromatography over silica (ethyl acetate/petroleum ether=1/2) to afford the title compound (480 mg, 35%). MS: 529.0 m/z [M+H]⁺.

### B. Ethyl 2-((3-((5-(3-((4,6-difluoro-1H-indol-5-yl)oxy)phenyl)-4H-1,2,4-triazol-3-yl)methyl)phenyl)thio)acetate

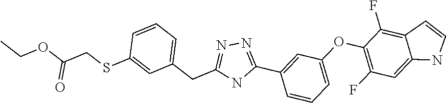

4,6-Difluoro-5-(3-(5-(3-iodobenzyl)-4H-1,2,4-triazol-3-yl)phenoxy)-1H-indole (80 mg, 0.15 mmol), Pd₂(dba)₃ (14 mg, 0.015 mmol), Xantphos (9 mg, 0.015 mol), diisopropylethylamine (40 mg, 0.30 mmol), dry dioxane (3 mL) and ethyl 2-mercaptoacetate (22 mg, 0.18 mmol) were added to a 5 mL reaction tube in a glove box, the tube was sealed and stirred at 100° C. overnight. The reaction mixture was cooled to room temperature, filtered through a pad of Celite and rinsed with ethyl acetate (20 mL×3). The filtrate was washed with water (20 mL) and brine (20 mL), dried over sodium sulfate, filtered and concentrated. The resulting residue was purified by flash chromatography over silica (50%-65% ethyl acetate in petroleum ether) to afford the title compound as a yellow solid (65 mg, 83%). MS: 521 m/z [M+H]⁺. ¹H NMR (400 MHz, CD₃OD) δ 7.69 (d, J=7.1 Hz, 1H), 7.55 (s, 1H), 7.43 (t, J=7.4 Hz, 1H), 7.36-7.24 (m, 4H), 7.17 (t, J=9.1 Hz, 2H), 7.04 (d, J=6.8 Hz, 1H), 6.57 (d, J=3.1 Hz, 1H), 4.13 (s, 2H), 4.04 (q, J=7.1 Hz, 2H), 3.68 (s, 2H), 1.09 (t, J=7.1 Hz, 3H) ppm.

### C. Example 261. 2-((3-((5-(3-((4,6-Difluoro-1H-indol-5-yl)oxy)phenyl)-4H-1,2,4-triazol-3-yl)methyl)phenyl)thio)acetic acid

To a stirred solution of ethyl 2-((3-((5-(3-((4,6-difluoro-1H-indol-5-yl)oxy)phenyl)-4H-1,2,4-triazol-3-yl)methyl)phenyl)thio)acetate (65 mg, 0.125 mmol) in a 1:1 mixture of THF and methanol (2 mL) was added a solution of lithium hydroxide in water (1M, 1 mL, 1.0 mmol). The reaction mixture was stirred at room temperature for two hours and then concentrated below 25° C. to remove THF and methanol. The aqueous solution was cooled to 0° C. and acidified with hydrochloric acid (1 M) to pH˜4. The solid was collected by filtration, washed with water (3 mL) and dried to afford the title compound as a light yellow solid (25 mg, 41%). MS: 493 m/z [M+H]⁺. ¹H NMR (400 MHz, DMSO-d₆) δ 14.07 (br s, 1H), 12.80 (s, 1H), 11.65 (s, 1H), 7.69 (d, J=7.6 Hz, 1H), 7.52-7.37 (m, 3H), 7.33 (d, J=10.3 Hz, 1H), 7.25-7.19 (m, 2H), 7.06 (t, J=8.0 Hz, 2H), 6.58 (s, 1H), 4.07 (s, 2H), 3.79 (s, 2H) ppm.

### Example 262

### 3-(3-((2-(3-((4-(Acetamidomethyl)-1H-indol-5-yl)oxy)phenyl)-1H-imidazol-5-yl)(hydroxy)methyl)phenyl)propanoic acid

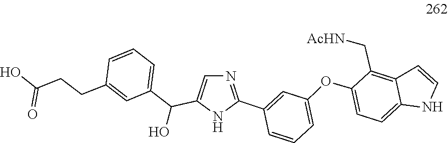 [SMILES: CC(=O)NCc1c(Oc2cccc(-c3ncc(C(O)c4cccc(CCC(=O)O)c4)[nH]3)c2)ccc2[nH]ccc12]

### A. Methyl 3-(3-(hydroxy(2-(3-((4-(hydroxymethyl)-1H-indol-5-yl)oxy)phenyl)-1H-imidazol-5-yl)methyl)phenyl)propanoate

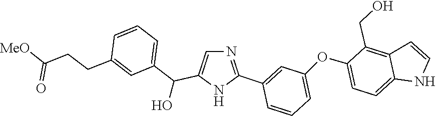 [SMILES: COC(=O)CCc1cccc(C(O)c2cnc(-c3cccc(Oc4ccc5[nH]ccc5c4CO)c3)[nH]2)c1]

A mixture of 3-((4-(hydroxymethyl)-1H-indol-5-yl)oxy)benzimidamide (2.1 g, 7.47 mmol, Intermediate 24-13) and methyl 3-(3-(3-formyloxiran-2-yl)phenyl)propanoate (2.62 g, 11.2 mmol, Intermediate 26-2) in DMF (20 mL) was stirred at 75° C. overnight. The reaction mixture was cooled to room temperature, diluted with water (100 mL) and extracted with ethyl acetate (50 mL×4). The combined organic extracts were washed with brine (50 mL×2), dried over sodium sulfate, filtered and concentrated. The resulting residue was purified by flash chromatography over silica (10% methanol in dichloromethane) to afford the title compound as a light yellow solid (845 mg, 21% from 2 steps). MS: 496 m/z [M−H]⁻.

### B. Methyl 3-(3-(2-(3-((4-formyl-1H-indol-5-yl)oxy)phenyl)-1H-imidazole-5-carbonyl)phenyl)propanoate

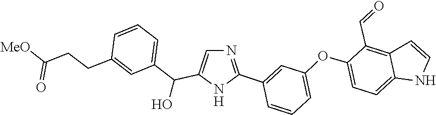 [SMILES: COC(=O)CCc1cccc(C(O)c2cnc(-c3cccc(Oc4ccc5[nH]ccc5c4C=O)c3)[nH]2)c1]

To a stirred solution of methyl 3-(3-(hydroxy(2-(3-((4-(hydroxymethyl)-1H-indol-5-yl)oxy)phenyl)-1H-imidazol-5-yl)methyl)phenyl)propanoate (850 mg, 1.71 mmol) in THF (20 mL) was added manganese dioxide (1.49 g, 17.1 mmol) at room temperature. The reaction mixture was stirred at room temperature overnight, filtered through a pad of Celite and concentrated to afford the title compound as a yellow solid (780 mg, crude), which was used in the next step without further purification. MS: 494 m/z [M+H]⁺.

### C. Methyl 3-(3-(2-(3-((4-((hydroxyimino)methyl)-1H-indol-5-yl)oxy)phenyl)-1H-imidazole-5-carbonyl)phenyl)propanoate

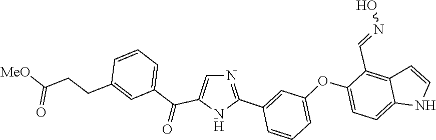 [SMILES: COC(=O)CCc1cccc(C(=O)c2cnc(-c3cccc(Oc4ccc5[nH]ccc5c4C=NO)c3)[nH]2)c1]

To a stirred solution of methyl 3-(3-(2-(3-((4-formyl-1H-indol-5-yl)oxy)phenyl)-1H-imidazole-5-carbonyl)phenyl)propanoate (400 mg, 0.81 mmol) in ethanol (5 mL) were added hydroxylamine hydrochloride (111 mg, 1.62 mmol) and sodium acetate (198 mg, 2.42 mmol). The reaction mixture was stirred at room temperature for five hours and then concentrated. The resulting residue was dissolved in ethyl acetate (50 mL), washed with water (10 mL×2) and brine (10 mL), dried over sodium sulfate, filtered and concentrated. The crude product was purified by flash chromatography over silica (dichloromethane/methanol, v/v, 10/1) to afford the title compound (322 mg, 78% from 2 steps). MS: 509 m/z [M+H]⁺.

### D. Methyl 3-(3-(2-(3-((4-(aminomethyl)-1H-indol-5-yl)oxy)phenyl)-1H-imidazole-5-carbonyl)phenyl)propanoate

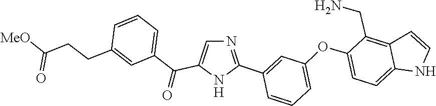 [SMILES: COC(=O)CCc1cccc(C(=O)c2cnc(-c3cccc(Oc4ccc5[nH]ccc5c4CN)c3)[nH]2)c1]

To a stirred solution of methyl 3-(3-(2-(3-((4-((hydroxyimino)methyl)-1H-indol-5-yl)oxy)phenyl)-1H-imidazole-5-carbonyl)phenyl)propanoate (320 mg, 0.63 mmol) in acetic acid (5 mL) and ethanol (5 mL) was added Raney Ni (150 mg). The reaction mixture was stirred at room temperature under hydrogen atmosphere overnight, filtered through a pad of Celite, and the filtrate was concentrated to afford the title compound as a yellow solid (168 mg, crude), which was used without further purification. MS: 495 m/z [M+H]⁺.

### E. Methyl 3-(3-(2-(3-((4-(acetamidomethyl)-1H-indol-5-yl)oxy)phenyl)-1H-imidazole-5-carbonyl)phenyl)propanoate

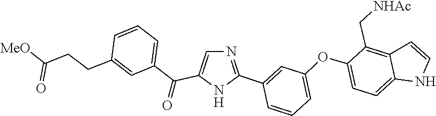 [SMILES: COC(=O)CCc1cccc(C(=O)c2cnc(-c3cccc(Oc4ccc5[nH]ccc5c4CNC(C)=O)c3)[nH]2)c1]

To a stirred solution of methyl 3-(3-(2-(3-((4-(aminomethyl)-1H-indol-5-yl)oxy)phenyl)-1H-imidazole-5-carbonyl)phenyl)propanoate (168 mg, 0.36 mmol) in THF (5 mL) was added acetic acid (26 mg, 0.42 mmol), HATU (203 mg, 0.53 mmol) and diisopropylethylamine (137 mg, 1.06 mmol). The reaction mixture was stirred at room temperature overnight, quenched with water (10 mL) and extracted with ethyl acetate (20 mL×3). The combined organic extracts were dried over sodium sulfate, filtered and concentrated to afford the title compound as a yellow solid (142 mg, crude), which was used without further purification. MS: 537 m/z [M+H]⁺.

### F. Methyl 3-(3-((2-(3-((4-(acetamidomethyl)-1H-indol-5-yl)oxy)phenyl)-1H-imidazol-5-yl)(hydroxy)methyl)phenyl)propanoate

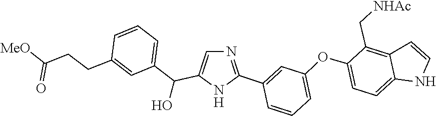 [SMILES: COC(=O)CCc1cccc(C(O)c2cnc(-c3cccc(Oc4ccc5[nH]ccc5c4CNC(C)=O)c3)[nH]2)c1]

To a stirred solution of methyl 3-(3-(2-(3-((4-(acetamidomethyl)-1H-indol-5-yl)oxy)phenyl)-1H-imidazole-5-carbonyl)phenyl)propanoate (142 mg, 0.27 mmol) in methanol (5 mL) was added sodium borohydride (30 mg, 0.80 mmol). The reaction mixture was stirred at 25° C. for three hours, quenched with saturated ammonium chloride (20 mL) and extracted with ethyl aceate (20 mL×3). The combined organic extracts were washed with brine (20 mL), dried over sodium sulfate, filtered and concentrated. The resulting residue was purified by preparative TLC to afford the title compound as a white solid (105 mg, 24%). MS: 539 m/z [M+H]⁺.

### G. Example 262. 3-(3-((2-(3-((4-(Acetamidomethyl)-1H-indol-5-yl)oxy)phenyl)-1H-imidazol-5-yl)(hydroxy)methyl)phenyl)propanoic acid

To a stirred solution of methyl 3-(3-((2-(3-((4-(acetamidomethyl)-1H-indol-5-yl)oxy)phenyl)-1H-imidazol-5-yl)(hydroxy)methyl)phenyl)propanoate (100 mg, 0.32 mmol) in THF (3 mL) was added a solution of lithium hydroxide (31 mg, 1.28 mmol) in water (1 mL). The reaction mixture was stirred at 60° C. for four hours, cooled to room temperature, diluted with water (10 mL), acidified with 1 N hydrochloric acid to pH˜2 and extracted with ethyl acetate (30 mL×4). The combined organic extracts were washed with brine, dried over sodium sulfate, filtered and concentrated. The resulting residue was purified by preparative HPLC to afford the title compound as a white solid (35 mg, 33%). MS: 525 m/z [M+H]⁺. ¹H NMR (400 MHz, CD₃OD) δ 7.47-7.31 (m, 6H), 7.26-7.23 (m, 2H), 7.18-7.15 (m, 1H), 6.98 (dd, J=8.0, 2.0 Hz, 1H), 6.85 (d, J=8.8 Hz, 1H), 6.75 (s, 1H), 6.59 (d, J=3.2 Hz, 1H), 5.74 (s, 1H), 4.64 (s, 2H), 2.91 (t, J=8.0 Hz, 2H), 2.48 (t, J=8.0 Hz, 2H), 1.72 (s, 3H) ppm.

### Example 263

### 3-(3-((2-(3-((4-((tert-Butylamino)methyl)-1H-indol-5-yl)oxy)phenyl)-1H-imidazol-5-yl)(hydroxy)methyl)phenyl)propanoic acid

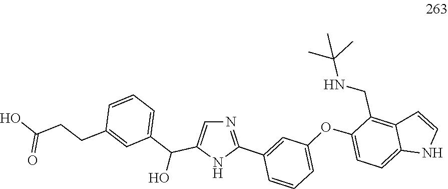 [SMILES: CC(C)(C)NCc1c(Oc2cccc(-c3ncc(C(O)c4cccc(CCC(=O)O)c4)[nH]3)c2)ccc2[nH]ccc12]

### A. Methyl 3-(3-(2-(3-((4-((tert-butylamino)methyl)-1H-indol-5-yl)oxy)phenyl)-1H-imidazole-5-carbonyl)phenyl)propanoate

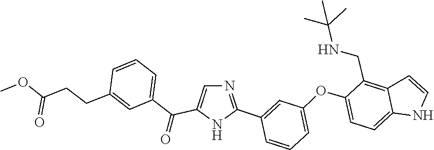 [SMILES: COC(=O)CCc1cccc(C(=O)c2cnc(-c3cccc(Oc4ccc5[nH]ccc5c4CNC(C)(C)C)c3)[nH]2)c1]

A mixture of methyl 3-(3-(2-(3-((4-formyl-1H-indol-5-yl)oxy)phenyl)-1H-imidazole-5-carbonyl)phenyl)propanoate (180 mg, 0.13 mmol) and 2-methylpropan-2-amine (11 mg, 0.19 mmol) in dichloromethane (3 mL) and methanol (1 mL) was stirred at room temperature for three hours and then sodium cyanoborohydride (24 mg, 0.38 mmol) was added. The reaction mixture was stirred at room temperature overnight, diluted with dichloromethane (50 mL), washed with brine (10 mL), dried over sodium sulfate and concentrated. The resulting residue was purified by preparative HPLC to afford the title compound as a white solid (180 mg, crude). MS: 551 m/z [M+H]⁺.

### B. Methyl 3-(3-((2-(3-((4-((tert-butylamino)methyl)-1H-indol-5-yl)oxy)phenyl)-1H-imidazol-5-yl)(hydroxy)methyl)phenyl)propanoate

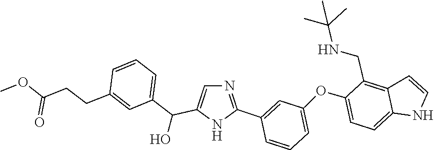 [SMILES: COC(=O)CCc1cccc(C(O)c2cnc(-c3cccc(Oc4ccc5[nH]ccc5c4CNC(C)(C)C)c3)[nH]2)c1]

To a stirred solution of methyl 3-(3-(2-(3-((4-((tert-butylamino)methyl)-1H-indol-5-yl)oxy)phenyl)-1H-imidazole-5-carbonyl)phenyl)propanoate (180 mg, 0.33 mmol) in methanol (5 mL) was added sodium borohydride (25 mg, 0.65 mmol). The reaction mixture was stirred at 25° C. for three hours, quenched with saturated ammonium chloride (20 mL) and extracted with ethyl acetate (20 mL×3). The combined organic extracts were washed with brine (20 mL), dried over sodium sulfate, filtered and concentrated to afford the title compound as a white solid (180 mg, crude). MS: 553 m/z [M+H]⁺.

### C. Example 263. 3-(3-((2-(3-((4-((tert-Butylamino)methyl)-1H-indol-5-yl)oxy)phenyl)-1H-imidazol-5-yl)(hydroxy)methyl)phenyl)propanoic acid

To a stirred solution of methyl 3-(3-((2-(3-((4-((tert-butylamino)methyl)-1H-indol-5-yl)oxy)phenyl)-1H-imidazol-5-yl)(hydroxy)methyl)phenyl)propanoate (180 mg, 0.33 mmol) in THF (5 mL) was added a solution of lithium hydroxide (39 mg, 4.63 mmol) in water (1.5 mL). The reaction mixture was stirred at 70° C. for five hours, cooled to room temperature, diluted with water (10 mL), acidified with 1 N hydrochloric acid and then extracted with ethyl acetate (30 mL×4). The combined organic extracts were washed with brine, dried over sodium sulfate, filtered and concentrated. The resulting residue was purified by preparative HPLC to afford the title compound as a white solid (42 mg, 21% from 3 steps). MS: 539 m/z [M+H]⁺. ¹H NMR (400 MHz, CD₃OD) δ 7.55-7.51 (m, 2H), 7.47-7.40 (m, 3H), 7.30 (s, 1H), 7.23 (d, J=4.4 Hz, 2H), 7.16 (t, J=4.4 Hz, 1H), 7.03 (d, J=8 Hz, 1H), 6.90 (d, J=8.8 Hz, 1H), 6.83 (s, 1H), 6.75 (d, J=2.8 Hz, 1H), 5.74 (s, 1H), 4.38 (s, 2H), 2.90 (t, J=7.6 Hz, 2H), 2.42 (t, J=7.6 Hz, 2H), 1.45 (s, 9H) ppm.

### Example 264

### Methyl 3-(3-(1-(2-(3-((4-(acetamidomethyl)-1H-indol-5-yl)oxy)phenyl)-1H-imidazol-5-yl)-1-hydroxyethyl)phenyl)propanoate

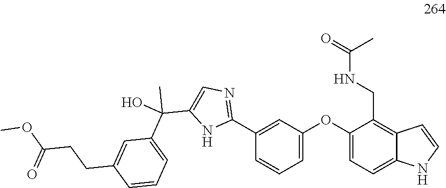 [SMILES: COC(=O)CCc1cccc(C(C)(O)c2cnc(-c3cccc(Oc4ccc5[nH]ccc5c4CNC(C)=O)c3)[nH]2)c1]

### A. Methyl 3-(3-(1-hydroxy-1-(2-(3-((4-(hydroxymethyl)-1H-indol-5-yl)oxy)phenyl)-1H-imidazol-5-yl)ethyl)phenyl)propanoate

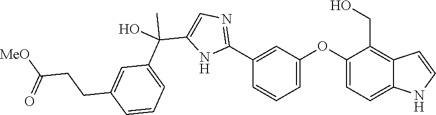 [SMILES: COC(=O)CCc1cccc(C(C)(O)c2cnc(-c3cccc(Oc4ccc5[nH]ccc5c4CO)c3)[nH]2)c1]

A mixture of 3-((4-(hydroxymethyl)-1H-indol-5-yl)oxy)benzimidamide (1.2 g, 4.27 mmol, Intermediate 24-13) and methyl 3-(3-(3-formyl-2-methyloxiran-2-yl)phenyl)propanoate (1.59 g, 6.41 mmol, Intermediate 26) in DMF (20 mL) was stirred at 75° C. overnight, cooled to room temperature, diluted with water (100 mL) and extracted with ethyl acetate (50 mL×4). The combined organic extracts were washed with brine (50 mL×2), dried over sodium sulfate, filtered and concentrated. The resulting residue was purified by flash chromatography over silica (10% methanol in dichloromethane) to afford the title compound as a light yellow solid (573 mg, 23%). MS: 512 m/z [M+H]⁺.

### B. Methyl 3-(3-(1-(2-(3-((4-formyl-1H-indol-5-yl)oxy)phenyl)-1H-imidazol-5-yl)-1-hydroxyethyl)phenyl)propanoate

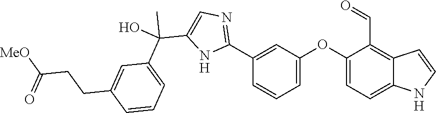 [SMILES: COC(=O)CCc1cccc(C(C)(O)c2cnc(-c3cccc(Oc4ccc5[nH]ccc5c4C=O)c3)[nH]2)c1]

To a stirred solution of methyl 3-(3-(1-hydroxy-1-(2-(3-((4-(hydroxymethyl)-1H-indol-5-yl)oxy)phenyl)-1H-imidazol-5-yl)ethyl)phenyl)propanoate (250 mg, 0.61 mmol) in THF (10 mL) was added manganese dioxide (531 mg, 6.11 mmol) at room temperature. The reaction mixture was stirred at room temperature overnight, filtered through a pad of Celite and the filtrate was concentrated to afford the title compound as a yellow solid (250 mg, crude), which was used without further purification.

### C. Methyl 3-(3-(1-hydroxy-1-(2-(3-((4-((hydroxyimino)methyl)-1H-indol-5-yl)oxy)phenyl)-1H-imidazol-5-yl)ethyl)phenyl)propanoate

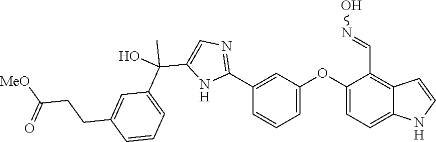 [SMILES: COC(=O)CCc1cccc(C(C)(O)c2cnc(-c3cccc(Oc4ccc5[nH]ccc5c4C=NO)c3)[nH]2)c1]

To a stirred solution of methyl 3-(3-(1-(2-(3-((4-formyl-1H-indol-5-yl)oxy)phenyl)-1H-imidazol-5-yl)-1-hydroxyethyl)phenyl)propanoate (190 mg, 0.76 mmol) in ethanol (5 mL) was added hydroxylamine hydrochloride (105 mg, 1.52 mmol) and sodium acetate (187 mg, 2.29 mmol). The reaction mixture was stirred at room temperature for five hours and concentrated. The residue was dissolved in ethyl acetate (50 mL), washed with water (10 mL×2) and brine (10 mL), dried over sodium sulfate, filtered and concentrated. The resulting residue was purified by flash chromatography over silica (dichloromethane/methanol, v/v, 10/1) to afford the title compound (173 mg, 83% from 2 steps). MS: 525 m/z [M+H]⁺.

### D. Methyl 3-(3-(1-(2-(3-((4-(aminomethyl)-1H-indol-5-yl)oxy)phenyl)-1H-imidazol-5-yl)-1-hydroxyethyl)phenyl)propanoate

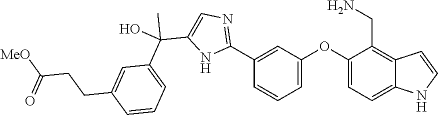 [SMILES: COC(=O)CCc1cccc(C(C)(O)c2cnc(-c3cccc(Oc4ccc5[nH]ccc5c4CN)c3)[nH]2)c1]

To a stirred solution of methyl 3-(3-(1-hydroxy-1-(2-(3-((4-((hydroxyimino)methyl)-1H-indol-5-yl)oxy)phenyl)-1H-imidazol-5-yl)ethyl)phenyl)propanoate (140 mg, 0.64 mmol) in acetic acid (5 mL) and ethanol (5 mL) was added Raney Ni (100 mg). The reaction mixture was stirred at room temperature under hydrogen atmosphere overnight, filtered through a pad of Celite and the filtrate was concentrated to afford the crude title compound as a yellow solid (73 mg), which was used without further purification.

### E. Example 264. Methyl 3-(3-(1-(2-(3-((4-(acetamidomethyl)-1H-indol-5-yl)oxy)phenyl)-1H-imidazol-5-yl)-1-hydroxyethyl)phenyl)propanoate

To a stirred solution of methyl 3-(3-(1-(2-(3-((4-(aminomethyl)-1H-indol-5-yl)oxy)phenyl)-1H-imidazol-5-yl)-1-hydroxyethyl)phenyl)propanoate (70 mg, 0.36 mmol) in THF (5 mL) was added acetic acid (26 mg, 0.42 mmol), HATU (203 mg, 0.53 mmol) and diisopropylethylamine (137 mg, 1.06 mmol). The reaction mixture was stirred at room temperature overnight, quenched with water (10 mL) and extracted with ethyl acetate (20 mL×3). The combined organic extracts were dried over sodium sulfate, filtered and concentrated to afford the title compound as a yellow solid (53 mg, crude). MS: 553 m/z [M+H]⁺. ¹H NMR (400 MHz, CD₃OD) δ 7.45-7.33 (m, 8H), 7.25-7.19 (m, 1H), 7.12-7.06 (m, 1H), 6.99-6.90 (m, 1H), 6.84 (d, J=8.6 Hz, 1H), 6.59 (d, J=2.5 Hz, 1H), 4.63 (s, 2H), 3.61 (s, 3H), 2.90 (t, J=6.7 Hz, 2H), 2.61 (t, J=7.6 Hz, 2H), 1.86 (s, 3H), 1.70 (s, 3H) ppm.

### Example 265

### 3-(3-((2-(2-Fluoro-5-((6-fluoro-4-(((2,2,2-trifluoroethyl)amino)methyl)-1H-indol-5-yl)oxy)phenyl)-1H-imidazol-5-yl)methyl)phenyl)propanoic acid

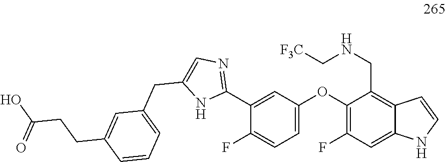 [SMILES: O=C(O)CCc1cccc(Cc2cnc(-c3cc(Oc4c(F)cc5[nH]ccc5c4CNCC(F)(F)F)ccc3F)[nH]2)c1]

### A. Methyl 3-(3-((2-(2-fluoro-5-((6-fluoro-4-(hydroxymethyl)-1H-indol-5-yl)oxy)phenyl)-1H-imidazol-5-yl)methyl)phenyl)propanoate

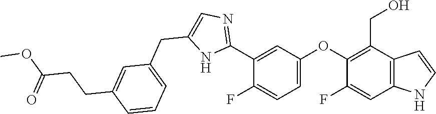 [SMILES: COC(=O)CCc1cccc(Cc2cnc(-c3cc(Oc4c(F)cc5[nH]ccc5c4CO)ccc3F)[nH]2)c1]

To a stirred solution of methyl 3-(3-(3-bromo-2-oxopropyl)phenyl)propanoate (380 mg, 1.2 mmol, Intermediate 21) in DMF (10 mL) were added 2-fluoro-5-((6-fluoro-4-(hydroxymethyl)-1H-indol-5-yl)oxy)benzimidamide (403 mg, 1.2 mmol, Intermediate 24-1) and sodium bicarbonate (201 mg, 2.4 mmol). The reaction mixture was heated at 80° C. overnight, cooled to room temperature, diluted with ethyl acetate (50 mL), washed with water (50 mL×2) and brine, dried over sodium sulfate and concentrated. The resulting residue was purified by flash chromatography (0-40% ethyl acetate in petroleum ether) to afford the title compound as a yellow solid (391 mg, 60%). MS: 518 m/z [M+H]⁺.

### B. Methyl 3-(3-((2-(2-fluoro-5-((6-fluoro-4-formyl-1H-indol-5-yl)oxy)phenyl)-1H-imidazol-5-yl)methyl)phenyl)propanoate

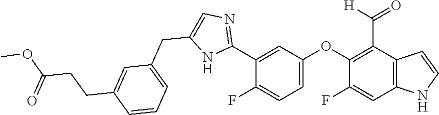 [SMILES: COC(=O)CCc1cccc(Cc2cnc(-c3cc(Oc4c(F)cc5[nH]ccc5c4C=O)ccc3F)[nH]2)c1]

To a stirred solution of methyl 3-(3-((2-(2-fluoro-5-((6-fluoro-4-(hydroxymethyl)-1H-indol-5-yl)oxy)phenyl)-1H-imidazol-5-yl)methyl)phenyl)propanoate (391 mg, 0.57 mmol) in THF (30 mL) was added manganese dioxide (1.3 g, 15.0 mmol). The reaction mixture was stirred at room temperature for twenty four hours under nitrogen, filtered through a pad of Celite, and concentrated. The resulting crude product was purified by silica gel column (0-80% ethyl acetate in petroleum ether) to afford the title compound as a yellow solid (230 mg, 58%). MS: 516 m/z [M+H]⁺.

### C. Methyl 3-(3-((2-(2-fluoro-5-((6-fluoro-4-(((2,2,2-trifluoroethyl)amino)methyl)-1H-indol-5-yl)oxy)phenyl)-1H-imidazol-5-yl)methyl)phenyl)propanoate

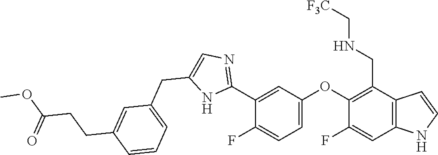 [SMILES: COC(=O)CCc1cccc(Cc2cnc(-c3cc(Oc4c(F)cc5[nH]ccc5c4CNCC(F)(F)F)ccc3F)[nH]2)c1]

To a stirred solution of methyl 3-(3-((2-(2-fluoro-5-((6-fluoro-4-formyl-1H-indol-5-yl)oxy)phenyl)-1H-imidazol-5-yl)methyl)phenyl)propanoate (210 mg, 0.4 mmol) in methanol (10 mL) was added trifluoroethylamine (0.6 mL, 2 M in THF). The reaction mixture was treated with three drops of acetic acid and stirred at room temperature for one hour. Sodium cyanoborohydride (126 mg, 2 mmol) was then added and stirred at room temperature for another sixteen hours. The reaction was quenched with water and extracted with ethyl acetate (60 mL×3). The combined organic extracts were washed with brine (20 mL), dried over sodium sulfate, filtered, and concentrated. The resulting residue was purified by silica gel column (0-80% ethyl acetate in petroleum ether) to afford the title compound as a yellow solid (122 mg, 50%). MS: 599 m/z [M+H]⁺.

### D. Example 265. 3-(3-((2-(2-Fluoro-5-((6-fluoro-4-(((2,2,2-trifluoroethyl)amino)methyl)-1H-indol-5-yl)oxy)phenyl)-1H-imidazol-5-yl)methyl)phenyl)propanoic acid

To a stirred solution of methyl 3-(3-((2-(2-fluoro-5-((6-fluoro-4-(((2,2,2-trifluoroethyl)amino)methyl)-1H-indol-5-yl)oxy)phenyl)-1H-imidazol-5-yl)methyl)phenyl)propanoate (122 mg, 0.3 mmol) in THF (5 mL) was added lithium hydroxide (24 mg, 0.6 mmol). The reaction mixture was stirred at room temperature for two hours, cooled to 0° C., acidified with 1M hydrochloric acid to pH˜4 and extracted with ethyl acetate (20 mL×3). The combined organic extracts were washed with brine (10 mL×2), dried over sodium sulfate, filtered and concentrated to afford the title compound as a white solid (76.1 mg, 64%). MS: 585 m/z [M+H]⁺. ¹H NMR (400 MHz, CD₃OD) δ 7.43 (dd, J=5.6, 3.2 Hz, 1H), 7.35 (d, J=3.2 Hz, 1H), 7.26 (d, J=10.8 Hz, 1H), 7.22-7.17 (m, 2H), 7.14 (s, 1H), 7.08 (d, J=7.2 Hz, 2H), 6.95 (dt, J=8.8, 3.6 Hz, 1H), 6.77 (s, 1H), 6.68 (d, J=3.2 Hz, 1H), 4.12 (s, 2H), 3.93 (s, 2H), 3.18 (q, J=10.0 Hz, 2H), 2.89 (t, J=7.6 Hz, 2H), 2.57 (t, J=7.6 Hz, 2H) ppm.

### Example 266

### 3-(3-((2-(3-((4,6-Difluoro-1H-indol-5-yl)oxy)phenyl)-1H-imidazol-5-yl)(methylamino)methyl)phenyl)propanoic acid

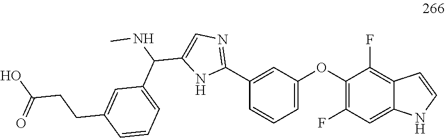 [SMILES: CNC(c1cccc(CCC(=O)O)c1)c1cnc(-c2cccc(Oc3c(F)cc4[nH]ccc4c3F)c2)[nH]1]

### A. Methyl 3-(3-((2-(3-((4,6-difluoro-1H-indol-5-yl)oxy)phenyl)-1H-imidazol-5-yl)(hydroxy)methyl)phenyl)propanoate

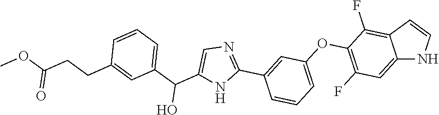 [SMILES: COC(=O)CCc1cccc(C(O)c2cnc(-c3cccc(Oc4c(F)cc5[nH]ccc5c4F)c3)[nH]2)c1]

To a stirred solution of 3-((4,6-difluoro-1H-indol-5-yl)oxy)benzimidamide (900 mg, 3.13 mmol, Intermediate 24-2) in DMF (10 mL) was added methyl 3-(3-(3-formyloxiran-2-yl)phenyl)propanoate (731 mg, 3.13 mmol, Intermediate 26-2). The resulting mixture was stirred at 70° C. overnight, cooled to room temperature, quenched with water (40 mL) and extracted with ethyl acetate (40 mL×3). The combined organic extracts were washed with brine (20 mL×2), dried over sodium sulfate, filtered and concentrated to give a residue which was purified by flash chromatography over silica (50% ethyl acetate in petroleum ether) to afford the title compound (600 mg, 38%). MS: 504 m/z [M+H]⁺.

### B. Methyl 3-(3-(2-(3-((4,6-difluoro-1H-indol-5-yl)oxy)phenyl)-1H-imidazole-5-carbonyl)phenyl)propanoate

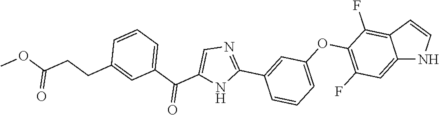 [SMILES: COC(=O)CCc1cccc(C(=O)c2cnc(-c3cccc(Oc4c(F)cc5[nH]ccc5c4F)c3)[nH]2)c1]

To a stirred solution of methyl 3-(3-((2-(3-((4,6-difluoro-1H-indol-5-yl)oxy)phenyl)-1H-imidazol-5-yl)(hydroxy)methyl)phenyl)propanoate (324 mg, 0.64 mmol) in THF (15 mL) was added manganese dioxide (560 mg, 6.44 mmol). The reaction mixture was stirred at room temperature overnight, filtered and the filtrate was concentrated to afford the title compound as a light yellow oil (314 mg, 96%), which was used without further purification. MS: 502 m/z [M+H]⁺.

### C. Methyl 3-(3-((2-(3-((4,6-difluoro-1H-indol-5-yl)oxy)phenyl)-1H-imidazol-5-yl)(methylamino)methyl)phenyl)propanoate

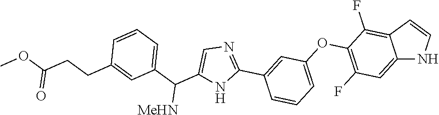 [SMILES: CNC(c1cccc(CCC(=O)OC)c1)c1cnc(-c2cccc(Oc3c(F)cc4[nH]ccc4c3F)c2)[nH]1]

A mixture of methyl 3-(3-(2-(3-((4,6-difluoro-1H-indol-5-yl)oxy)phenyl)-1H-imidazole-5-carbonyl)phenyl)propanoate (150 mg, 0.30 mmol), methylamine in THF (1.5 mL, 2.99 mmol, 2 N in THF) and titanium tetraisopropanolate (850 mg, 2.99 mmol) in methanol (10 mL) was stirred in a sealed tube at 50° C. overnight. The reaction mixture was cooled to 0° C., sodium borohydride (35 mg, 0.90 mmol) added, stirred at room temperature for another two hours, quenched with water (10 mL) and concentrated. The resulting residue was diluted with ethyl acetate (100 mL), washed with water (20 mL×2) and brine (20 mL×1), dried over sodium sulfate, filtered and concentrated. The resulting crude product was purified by preparative TLC (10% methanol in dichloromethane) to afford the title compound as a light yellow solid (130 mg, 83%). MS: 515 m/z [M−H]⁻.

### D. Example 266. 3-(3-((2-(3-((4,6-Difluoro-1H-indol-5-yl)oxy)phenyl)-1H-imidazol-5-yl)(methylamino)methyl)phenyl)propanoic acid

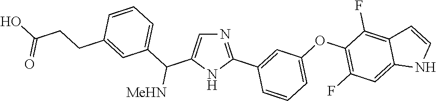 [SMILES: CNC(c1cccc(CCC(=O)O)c1)c1cnc(-c2cccc(Oc3c(F)cc4[nH]ccc4c3F)c2)[nH]1]

To a stirred solution of methyl 3-(3-((2-(3-((4,6-difluoro-1H-indol-5-yl)oxy)phenyl)-1H-imidazol-5-yl)(methylamino)methyl)phenyl)propanoate (130 mg, 0.25 mmol) in THF (10 mL) was added a solution of lithium hydroxide (31 mg, 1.26 mmol) in water (0.5 mL). The reaction mixture was stirred at 60° C. for three hours, pH adjusted to ˜5 with 1N hydrochloric acid and the mixture was concentrated. The resulting crude was purified by preparative HPLC to afford the title compound as a white solid (45 mg, 35%). MS: 503 m/z [M+H]⁺. ¹H NMR (400 MHz, CD₃OD) δ 7.59 (d, J=8.0 Hz, 1H), 7.53 (s, 1H), 7.47 (s, 1H), 7.44-7.40 (m, 1H), 7.35-7.31 (m, 4H), 7.18-7.16 (m, 1H), 7.02-6.98 (m, 2H), 6.57 (d, J=3.2 Hz, 1H), 5.23 (s, 1H), 2.94 (t, J=7.6 Hz, 2H), 2.56 (s, 3H), 2.48 (t, J=7.2 Hz, 2H) ppm.

### Example 267

### 3-(3-((2-(3-((6-Fluoro-4-(1H-pyrazol-5-yl)-1H-indol-5-yl)oxy)phenyl)-1H-imidazol-5-yl)(hydroxy)methyl)phenyl)propanoic acid

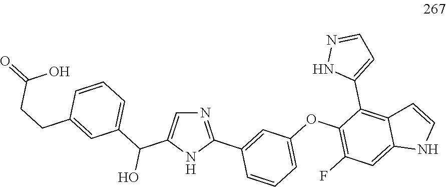 [SMILES: O=C(O)CCc1cccc(C(O)c2cnc(-c3cccc(Oc4c(F)cc5[nH]ccc5c4-c4ccn[nH]4)c3)[nH]2)c1]

### A. Methyl 3-(3-((2-(3-((4-bromo-6-fluoro-1H-indol-5-yl)oxy)phenyl)-1H-imidazol-5-yl)(hydroxy)methyl)phenyl)propanoate

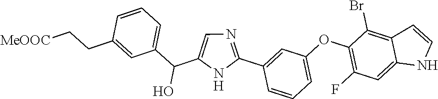 [SMILES: COC(=O)CCc1cccc(C(O)c2cnc(-c3cccc(Oc4c(F)cc5[nH]ccc5c4Br)c3)[nH]2)c1]

A mixture of 3-((4-bromo-6-fluoro-1H-indol-5-yl)oxy)benzimidamide (1.1 g, 3.16 mmol, Intermediate 24-3) and methyl 3-(3-(3-formyloxiran-2-yl)phenyl)propanoate (814 mg, 3.48 mmol, Intermediate 26-2) in DMF (15 mL) was stirred at 70° C. for sixteen hours. The reaction mixture was cooled to room temperature, diluted with ethyl acetate (100 mL), washed with water (20 mL×2) and brine (20 mL×2), dried over sodium sulfate and concentrated. The resulting residue was purified by flash chromatography over silica (petroleum ether/ethyl acetate/dichloromethane, v/v, 1/2/1) to afford the title compound as a yellow solid (1.02 g, 55%). MS: 564/566 m/z [M+H]⁺.

### B. Methyl 3-(3-((2-(3-((6-fluoro-4-(1H-pyrazol-5-yl)-1H-indol-5-yl)oxy)phenyl)-1H-imidazol-5-yl)(hydroxy)methyl)phenyl)propanoate

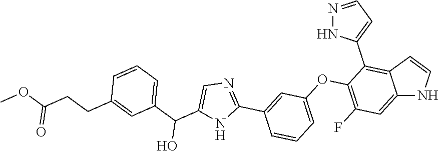 [SMILES: COC(=O)CCc1cccc(C(O)c2cnc(-c3cccc(Oc4c(F)cc5[nH]ccc5c4-c4ccn[nH]4)c3)[nH]2)c1]

A mixture of methyl 3-(3-((2-(3-((4-bromo-6-fluoro-1H-indol-5-yl)oxy)phenyl)-1H-imidazol-5-yl)(hydroxy)methyl)phenyl)propanoate (300 mg, 0.91 mmol), 3-(4,4,5,5-tetramethyl-1,3,2-dioxaborolan-2-yl)-1H-pyrazole (459 mg, 1.81 mmol), Pd(dppf)Cl₂ (66 mg, 0.09 mmol) and potassium acetate (222 mg, 2.26 mmol) in dioxane (8 mL) and water (0.5 mL) was stirred at 90° C. for twelve hours. The reaction mixture cooled to room temperature, diluted with ethyl acetate (100 mL), washed with water and brine, dried over sodium sulfate and concentrated. The resulting residue was purified by flash chromatography over silica (5% methanol in dichloromethane) to afford the title compound as a yellow solid (245 mg, 49%). MS: 552 m/z [M+H]⁺.

### C. Example 267. 3-(3-((2-(3-((6-Fluoro-4-(1H-pyrazol-5-yl)-1H-indol-5-yl)oxy)phenyl)-1H-imidazol-5-yl)(hydroxy)methyl)phenyl)propanoic acid

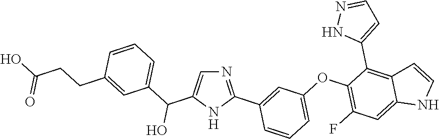 [SMILES: O=C(O)CCc1cccc(C(O)c2cnc(-c3cccc(Oc4c(F)cc5[nH]ccc5c4-c4ccn[nH]4)c3)[nH]2)c1]

To a stirred solution of methyl 3-(3-((2-(3-((6-fluoro-4-(1H-pyrazol-5-yl)-1H-indol-5-yl)oxy)phenyl)-1H-imidazol-5-yl)(hydroxy)methyl)phenyl)propanoate (70 mg, 0.13 mmol) in THF (10 mL) was added a solution of lithium hydroxide (9 mg, 0.38 mmol) in water (0.5 mL). The resulting mixture was stirred at 30° C. for three hours, diluted with water (8 mL), pH adjusted to ˜4 with 1N hydrochloric acid and extracted with ethyl acetate (40 mL×4). The combined organic extracts were washed with brine, dried over sodium sulfate, filtered and concentrated. The resulting residue was purified by preparative HPLC to afford the title compound as a white solid (21.8 mg, 32%). MS: 538 m/z [M+H]⁺. ¹H NMR (400 MHz, CD₃OD) δ 7.59 (s, 1H), 7.45-7.43 (m, 1H), 7.34-7.26 (m, 7H), 7.17 (br s, 1H), 6.83-6.81 (d, J=8.4 Hz, 1H), 6.77-6.73 (d, J=12.4 Hz, 1H), 6.59 (s, 1H), 5.76 (s, 1H), 2.94-2.91 (t, J=7.6 Hz, 2H), 2.59-2.56 (t, J=7.6 Hz, 2H) ppm.

### Example 268

### 3-(3-((2-(5-((4,6-Difluoro-1H-indol-5-yl)oxy)-2-fluorophenyl)-1H-imidazol-5-yl)methyl)-2-fluorophenyl)propan-1-ol

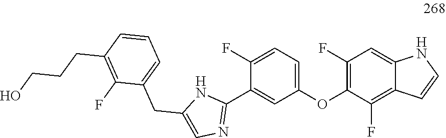 [SMILES: OCCCc1cccc(Cc2cnc(-c3cc(Oc4c(F)cc5[nH]ccc5c4F)ccc3F)[nH]2)c1F]

To a stirred and cooled (0° C.) solution of ethyl 3-(3-((2-(5-((4,6-difluoro-1H-indol-5-yl)oxy)-2-fluorophenyl)-1H-imidazol-5-yl)methyl)-2-fluorophenyl)propanoate (product of Step J, Example 100, 500 mg, 0.930 mmol) in THF (20 mL) was added lithium aluminum hydride (71 mg, 1.86 mmol). The reaction was stirred at room temperature for two hours. The mixture was then quenched with sodium sulfate decahydrate carefully and filtered. The filtrate was concentrated. The residue was purified by Prep-HPLC (5%-95% 0.05% ammonium carbonate in water and acetonitrile) to give the title compound as a white solid (312 mg, 65.4%). MS: 496 m/z [M+H]⁺. ¹H NMR (400 MHz, CD₃OD) δ 7.53 (s, 1H), 7.31 (d, J=3.2 Hz, 1H), 7.24-7.11 (m, 3H), 7.10-6.94 (m, 3H), 6.76 (s, 1H), 6.57 (d, J=3.2 Hz, 1H), 3.95 (s, 2H), 3.59 (t, J=6.4 Hz, 2H), 2.73 (t, J=7.6 Hz, 2H), 1.90-1.77 (m, 2H).

### Example 269

### 3-(3-((2-(5-((4,6-Difluoro-1H-indol-5-yl)oxy)-2-fluorophenyl)-1H-imidazol-5-yl)methyl)-2-fluorophenyl)propyl dihydrogen phosphate

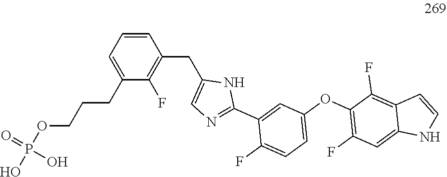 [SMILES: O=P(O)(O)OCCCc1cccc(Cc2cnc(-c3cc(Oc4c(F)cc5[nH]ccc5c4F)ccc3F)[nH]2)c1F]

To a stirred and cooled (0° C.) solution of phosphoryl trichloride (2.66 g, 17.4 mmol) and trimethylamine (3.51 g, 34.7 mmol) in THF (80 mL) was added 3-(3-((2-(5-((4,6-difluoro-1H-indol-5-yl)oxy)-2-fluorophenyl)-1H-imidazol-5-yl)methyl)-2-fluorophenyl)propan-1-ol (Example 268, 860 mg, 1.74 mmol) in THF (20 mL) dropwise under argon. The reaction was stirred at 0° C. for one hour. The mixture was then quenched with 5 mL of water, stirred for another 30 minutes and concentrated. The residue was purified by prep-HPLC to give the title compound as a white solid (204 mg, 20%). MS: 576 m/z [M+H]⁺. ¹H NMR (400 MHz, CD₃OD) δ 7.47 (dd, J=5.9, 3.1 Hz, 1H), 7.31 (d, J=3.2 Hz, 1H), 7.29-7.22 (m, 1H), 7.19-7.13 (m, 2H), 7.09-6.99 (m, 3H), 6.90 (s, 1H), 6.55 (d, J=3.1 Hz, 1H), 3.98 (s, 2H), 3.90 (q, J=7.6 Hz, 2H), 2.77 (t, J=7.6 Hz, 2H), 2.00-1.81 (m, 2H) ppm.

### Example 270

### (4,6-Difluoro-5-(4-fluoro-3-(4-(2-fluoro-3-(3-methoxy-3-oxopropyl)benzyl)-1H-imidazol-2-yl)phenoxy)-1H-indol-1-yl)phosphonic acid

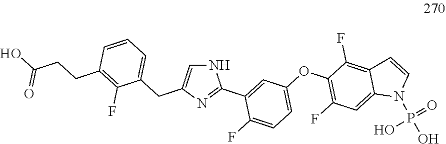 [SMILES: O=C(O)CCc1cccc(Cc2c[nH]c(-c3cc(Oc4c(F)cc5c(ccn5P(=O)(O)O)c4F)ccc3F)n2)c1F]

### A. Methyl 3-(3-((2-(5-((1-(bis(benzyloxy)phosphoryl)-4,6-difluoro-1H-indol-5-yl)oxy)-2-fluorophenyl)-1H-imidazol-4-yl)methyl)-2-fluorophenyl)propanoate

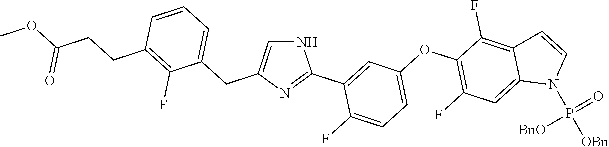 [SMILES: COC(=O)CCc1cccc(Cc2c[nH]c(-c3cc(Oc4c(F)cc5c(ccn5P(=O)(OCc5ccccc5)OCc5ccccc5)c4F)ccc3F)n2)c1F]

To a stirred and cooled (−78° C.) solution of ethyl 3-[3-[[2-[5-[(4,6-difluoro-1H-indol-5-yl)oxy]-2-fluoro-phenyl]-1H-imidazol-5-yl]methyl]-2-fluoro-phenyl]propanoate (product of Step J, Example 100, 250 mg, 0.48 mmol) in THF (20 mL) was added potassium bis(trimethylsilyl)amide in THF (1 M, 1.09 mL, 1.1 mmol) dropwise. The reaction was stirred at −78° C. for twenty minutes. To this reaction mixture was added dibenzyl phosphoryl chloride (0.33 g, 1.1 mmol) in THF (2 mL) dropwise and stirred at the same temperature for additional one hour. The cool reaction mixture was poured into water (50 mL), extracted with ethyl acetate (2×20 mL). The combined extract was washed with water (3×10 mL), brine (20 mL), dried over sodium sulfate, filtered and concentrated. The residue was purified by flash chromatography over silica (0-80% ethyl acetate in petroleum ether) to give the title compound as oil (0.06 g, 16%). MS: 784 m/z [M+H]⁺.

### B. (4,6-Difluoro-5-(4-fluoro-3-(4-(2-fluoro-3-(3-methoxy-3-oxopropyl)benzyl)-1H-imidazol-2-yl)phenoxy)-1H-indol-1-yl)phosphonic acid

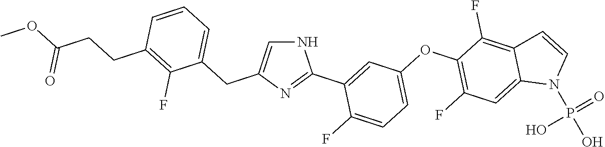 [SMILES: COC(=O)CCc1cccc(Cc2c[nH]c(-c3cc(Oc4c(F)cc5c(ccn5P(=O)(O)O)c4F)ccc3F)n2)c1F]

To a solution of methyl 3-(3-((2-(5-((1-(bis(benzyloxy)phosphoryl)-4,6-difluoro-1H-indol-5-yl)oxy)-2-fluorophenyl)-1H-imidazol-4-yl)methyl)-2-fluorophenyl)propanoate (60 mg) in methanol (10 mL) was added 10% Pd on carbon (20 mg). The mixture was purged with nitrogen and stirred under hydrogen atmosphere at room temperature for four hours. The mixture was filtered through Celite and the filter cake was rinsed with methanol (20 mL). The filtrate was concentrated. The residue was washed with ethyl ether (5 mL) and dried in vacuo to give the title compound as a solid (31.7 mg, 69%). ¹H NMR (400 MHz, CD₃OD) δ 7.74 (d, J=10.9 Hz, 1H), 7.63-7.55 (m, 1H), 7.49-7.06 (m, 7H), 6.60-6.58 (m, 1H), 4.13 (s, 2H), 3.64 (s, 3H), 2.98 (t, J=7.3 Hz, 2H), 2.65 (t, J=7.3 Hz, 2H). MS: 604 m/z [M+H]⁺.

### C. Example 270, 3-(3-((2-(5-((4,6-Difluoro-1-phosphono-1H-indol-5-yl)oxy)-2-fluorophenyl)-1H-imidazol-4-yl)methyl)-2-fluorophenyl)propanoic acid

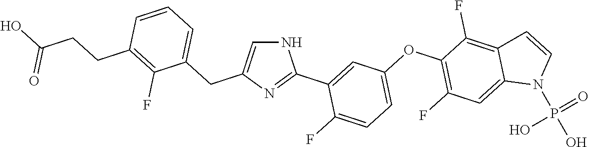 [SMILES: O=C(O)CCc1cccc(Cc2c[nH]c(-c3cc(Oc4c(F)cc5c(ccn5P(=O)(O)O)c4F)ccc3F)n2)c1F]

To a solution of (4,6-difluoro-5-(4-fluoro-3-(4-(2-fluoro-3-(3-methoxy-3-oxopropyl)benzyl)-1H-imidazol-2-yl)phenoxy)-1H-indol-1-yl)phosphonic acid (1.12 μg, 1.8 mmol) in tetrahydrofuran/water/methanol (3:1:1, 100 mL) was added lithium hydroxide (0.76 g, 18 mmol) The reaction was stirred at room temperature for two hours and acidified with 1N hydrochloric acid to pH˜5. The precipitate was collected by filtration. The crude product was recrystallized with ether and methanol to give the title compound as a white solid (0.77 g, 71%). ¹H NMR (400 MHz, DMSO-d₆) δ 7.76 (d, J=11.2 Hz, 1H), 7.57-7.43 (m, 3H), 7.30-7.06 (m, 5H), 5.65 (s, 1H), 4.06 (s, 2H), 2.86 (t, J=7.3 Hz, 2H), 2.56 (t, J=7.3 Hz, 2H),

### Example 271

### 3-(3-((2-(3-((6-Fluoro-4-(1H-pyrazol-5-yl)-1H-indol-5-yl)oxy)phenyl)-1H-imidazol-4-yl)methyl)phenyl)propanoic acid

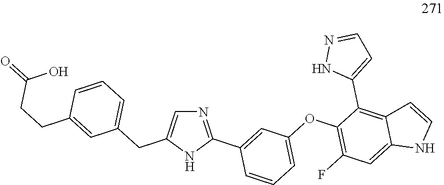 [SMILES: O=C(O)CCc1cccc(Cc2cnc(-c3cccc(Oc4c(F)cc5[nH]ccc5c4-c4ccn[nH]4)c3)[nH]2)c1]

### A. Methyl 3-(3-((2-(3-((6-fluoro-4-(1H-pyrazol-5-yl)-1H-indol-5-yl)oxy)phenyl)-1H-imidazol-5-yl)methyl)phenyl)propanoate

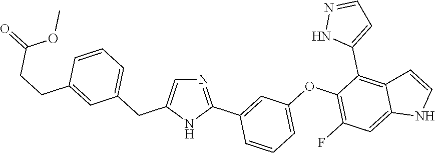 [SMILES: COC(=O)CCc1cccc(Cc2cnc(-c3cccc(Oc4c(F)cc5[nH]ccc5c4-c4ccn[nH]4)c3)[nH]2)c1]

A mixture of methyl 3-(3-((2-(3-((6-fluoro-4-(1H-pyrazol-5-yl)-1H-indol-5-yl)oxy)phenyl)-1H-imidazol-5-yl)(hydroxy)methyl)phenyl)propanoate (100 mg, 0.18 mmol) and 20% Pd(OH)₂ on carbon (50 mg) in a mixture of acetic acid (1 mL) and THF (12 mL) was stirred at room temperature under hydrogen atmosphere for two days and then filtered through a pad of Celite. The filtrate was concentrated and the resulting residue was purified by preparative TLC to afford the title compound as a white solid (25 mg, 26%). MS: 536 m/z [M+H]⁺.

### B. Example 271. 3-(3-((2-(3-((6-Fluoro-4-(1H-pyrazol-5-yl)-1H-indol-5-yl)oxy)phenyl)-1H-imidazol-5-yl)methyl)phenyl)propanoic acid

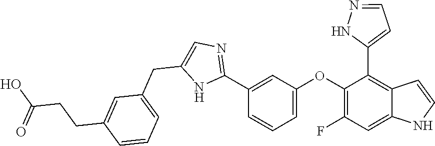 [SMILES: O=C(O)CCc1cccc(Cc2cnc(-c3cccc(Oc4c(F)cc5[nH]ccc5c4-c4ccn[nH]4)c3)[nH]2)c1]

To a stirred solution of methyl 3-(3-((2-(3-((6-fluoro-4-(1H-pyrazol-5-yl)-1H-indol-5-yl)oxy)phenyl)-1H-imidazol-5-yl)methyl)phenyl)propanoate (26 mg, 0.048 mmol) in THF (10 mL) was added a solution of lithium hydroxide (6 mg, 0.24 mmol) in water (0.5 mL). The reaction mixture was stirred at 30° C. for eight hours, diluted with water (8 mL), pH adjusted to˜4 with 1N hydrochloric acid and extracted with ethyl acetate (40 mL×4). The combined organic extracts were washed with brine, dried over sodium sulfate, filtered and concentrated.

The resulting residue was purified by preparative HPLC to afford the title compound as a white solid (13 mg, 51%). MS: 522 m/z [M+H]⁺. ¹H NMR (400 MHz, CD₃OD) δ 7.59 (d, J=1.2 Hz, 1H), 7.43 (d, J=7.6 Hz, 1H), 7.35-7.27 (m, 4H), 7.22-7.18 (m, 1H), 7.14 (s, 1H), 7.09-7.06 (m, 2H), 6.85-6.83 (m, 1H), 6.77 (d, J=2.8 Hz, 1H), 6.75 (s, 1H), 6.59 (d, J=1.6 Hz, 1H), 3.91 (s, 2H), 2.91-2.87 (m, 2H), 2.58-2.54 (m, 2H) ppm.

### Example 272

### 3-(6-(1-(2-(2-fluoro-5-((6-fluoro-4-methyl-1H-indol-5-yl)oxy)phenyl)-1H-imidazol-5-yl)-1-hydroxyethyl)pyridin-2-yl)propanoic acid

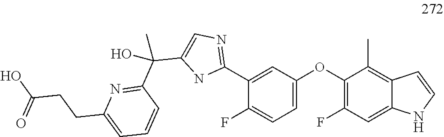 [SMILES: Cc1c(Oc2ccc(F)c(-c3ncc(C(C)(O)c4cccc(CCC(=O)O)n4)[nH]3)c2)c(F)cc2[nH]ccc12]

### A. Ethyl 3-(6-((2-(2-fluoro-5-((6-fluoro-4-methyl-1-tosyl-1H-indol-5-yl)oxy)phenyl)-1H-imidazol-5-yl)methyl)pyridin-2-yl)propanoate

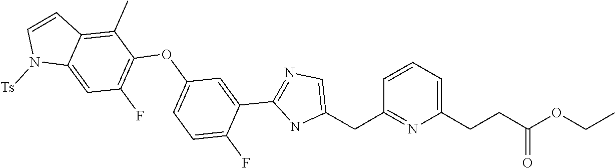 [SMILES: CCOC(=O)CCc1cccc(Cc2cnc(-c3cc(Oc4c(F)cc5c(ccn5S(=O)(=O)c5ccc(C)cc5)c4C)ccc3F)[nH]2)n1]

To a reaction tube in a glove box were added 2-fluoro-5-((6-fluoro-4-methyl-1-tosyl-1H-indol-5-yl)oxy)-N-(prop-2-yn-1-yl)benzimidamide (350 mg, 0.71 mmol, Example 89, Step C), ethyl 3-(6-bromopyridin-2-yl)propanoate (220 mg, 0.85 mmol), Pd(PPh₃)₄ (82 mg, 0.07 mmol), CuI (27 mg, 0.14 mmol), potassium carbonate (490 mg, 3.55 mmol) and DMF (10 mL). The mixture was stirred at 60° C. overnight, diluted with ethyl acetate (50 mL), washed with brine (15 mL×2), dried and concentrated. The residue was purified by flash chromatography (0-100% ethyl acetate in petroleum ether) to afford the title compound as a yellow solid (130 mg, yield 27%). MS: 671 m/z [M+H]⁺.

### B. Ethyl 3-(6-(2-(2-fluoro-5-((6-fluoro-4-methyl-1-tosyl-1H-indol-5-yl)oxy)phenyl)-1H-imidazole-5-carbonyl)pyridin-2-yl)propanoate

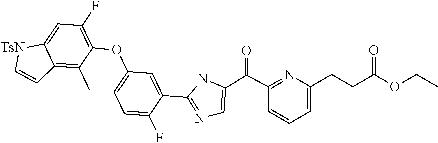 [SMILES: CCOC(=O)CCc1cccc(C(=O)c2cnc(-c3cc(Oc4c(F)cc5c(ccn5S(=O)(=O)c5ccc(C)cc5)c4C)ccc3F)[nH]2)n1]

To a stirred solution of ethyl 3-(6-((2-(2-fluoro-5-((6-fluoro-4-methyl-1-tosyl-1H-indol-5-yl)oxy)phenyl)-1H-imidazol-5-yl)methyl)pyridin-2-yl)propanoate (114 mg, 0.17 mmol) in ethyl acetate (5 mL) was added manganese dioxide (296 mg, 3.4 mmol). The reaction mixture was stirred at room temperature over weekend, filtered and rinsed with methanol (10 mL×2). The filtrate was concentrated to afford the crude title compound as a solid (101 mg, 87%). MS: 685 m/z [M+H]⁺.

### C. 3-(6-(2-(2-Fluoro-5-((6-fluoro-4-methyl-1H-indol-5-yl)oxy)phenyl)-1H-imidazole-5-carbonyl)pyridin-2-yl)propanoic acid

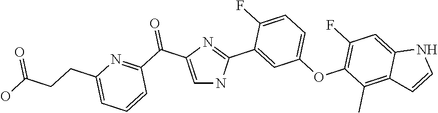 [SMILES: Cc1c(Oc2ccc(F)c(-c3nc(C(=O)c4cccc(CCC(=O)O)n4)c[nH]3)c2)c(F)cc2[nH]ccc12]

Using the conditions described in step E of Example 89, ethyl 3-(6-(2-(2-fluoro-5-((6-fluoro-4-methyl-1-tosyl-1H-indol-5-yl)oxy)phenyl)-1H-imidazole-5-carbonyl)pyridin-2-yl)propanoate (101 mg) was subjected to hydrolysis and deprotection by lithium hydroxide to afford the title compound as a solid (40 mg, 54%). MS: 503 m/z [M+H]⁺.

### D. Example 272. 3-(6-(1-(2-(2-Fluoro-5-((6-fluoro-4-methyl-1H-indol-5-yl)oxy)phenyl)-1H-imidazol-5-yl)-1-hydroxyethyl)pyridin-2-yl)propanoic acid

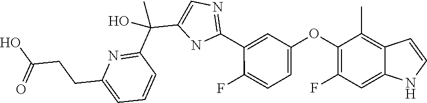 [SMILES: Cc1c(Oc2ccc(F)c(-c3ncc(C(C)(O)c4cccc(CCC(=O)O)n4)[nH]3)c2)c(F)cc2[nH]ccc12]

To a stirred solution of 3-(6-(2-(2-fluoro-5-((6-fluoro-4-methyl-1H-indol-5-yl)oxy)phenyl)-1H-imidazole-5-carbonyl)pyridin-2-yl)propanoic acid (60 mg, 0.12 mmol) in THF (6 mL) was added methylmagnesium bromide (3M in THF) (200 uL, 0.6 mmol) in a glove box. The mixture was stirred at room temperature for three hours, diluted with ethyl acetate (50 mL), washed with saturated ammonium chloride solution (10 mL×2) and brine, dried and concentrated. The resulting residue was purified by preparative HPLC to afford the title compound as a solid (18.9 mg, 30%). MS: 519 m/z [M+H]⁺. ¹H NMR (400 MHz, CD₃OD) δ 7.70-7.67 (m, 1H), 7.42-7.38 (m, 2H), 7.28 (d, J=3.2 Hz, 1H), 7.21-7.08 (m, 3H), 7.04 (s, 1H), 6.92-6.79 (m, 1H), 6.52 (d, J=3.1 Hz, 1H), 3.13 (t, J=7.1 Hz, 2H), 2.75 (t, J=7.1 Hz, 2H), 2.39 (s, 3H), 1.89 (s, 3H) ppm.

### Example 273

### 3-(3-(1-(2-(5-((4,6-difluoro-1H-indol-5-yl)oxy)-2-fluorophenyl)-1H-imidazol-5-yl)-2,2,2-trifluoro-1-hydroxyethyl)phenyl)propanoic acid

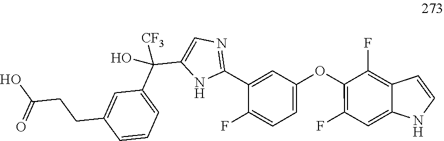 [SMILES: O=C(O)CCc1cccc(C(O)(c2cnc(-c3cc(Oc4c(F)cc5[nH]ccc5c4F)ccc3F)[nH]2)C(F)(F)F)c1]

### A. Methyl 3-(3-(2-(5-((4,6-difluoro-1H-indol-5-yl)oxy)-2-fluorophenyl)-1H-imidazole-5-carbonyl)phenyl)propanoate

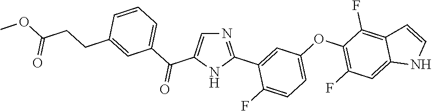 [SMILES: COC(=O)CCc1cccc(C(=O)c2cnc(-c3cc(Oc4c(F)cc5[nH]ccc5c4F)ccc3F)[nH]2)c1]

The title compound was prepared using the conditions described in steps A and B of Example 44, but exchanging 2-fluoro-5-((6-fluoro-4-(methylthio)-1H-indol-5-yl)oxy)benzimidamide for 5-((4,6-difluoro-1H-indol-5-yl)oxy)-2-fluorobenzimidamide (604 mg, 1.98 mmol) as a yellow syrup (290 mg). MS: 520 m/z [M+H]⁺.

### B. Methyl 3-(3-(2-(5-((4,6-difluoro-1-((2-(trimethylsilyl)ethoxy)methyl)-1H-indol-5-yl)oxy)-2-fluorophenyl)-1-((2-(trimethylsilyl)ethoxy)methyl)-1H-imidazole-5-carbonyl)phenyl)propanoate

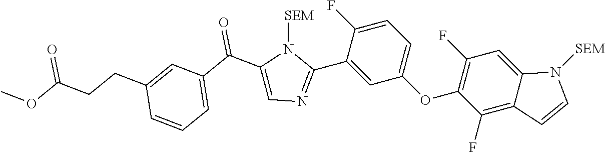 [SMILES: COC(=O)CCc1cccc(C(=O)c2cnc(-c3cc(Oc4c(F)cc5c(ccn5COCC[Si](C)(C)C)c4F)ccc3F)n2COCC[Si](C)(C)C)c1]

To a stirred solution of methyl 3-(3-(2-(5-((4,6-difluoro-1H-indol-5-yl)oxy)-2-fluorophenyl)-1H-imidazole-5-carbonyl)phenyl)propanoate (290 mg, 0.56 mmol) in THF (15 mL) was added sodium hydride (60% in mineral oil) (78 mg, 1.95 mmol) at 0° C. under nitrogen. The mixture was stirred at 0° C. for one hour, 2-(trimethylsilyl)ethoxymethyl chloride (325 mg, 1.95 mmol) was added and then stirred at room temperature for another hour. The reaction mixture was poured into an ice-water mixture and extracted with ethyl acetate (45 mL×3). The combined organic extracts were washed with brine (45 mL), dried over sodium sulfate, filtered and concentrated. The residue was purified by flash chromatography over silica (14-24% ethyl acetate in petroleum ether) to afford the title compound as light yellow syrup (270 mg, 61%). MS: 780 m/z [M+H]⁺.

### C. Methyl 3-(3-(1-(2-(5-((4,6-difluoro-1-((2-(trimethylsilyl)ethoxy)methyl)-1H-indol-5-yl)oxy)-2-fluorophenyl)-1-((2-(trimethylsilyl)ethoxy)methyl)-1H-imidazol-5-yl)-2,2,2-trifluoro-1-((trimethylsilyl)oxy)ethyl)phenyl)propanoate

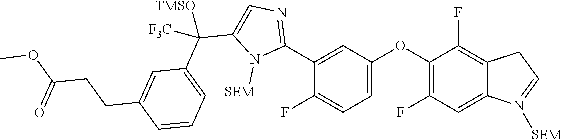

To a stirred solution of methyl 3-(3-(2-(5-((4,6-difluoro-1-((2-(trimethylsilyl)ethoxy)methyl)-1H-indol-5-yl)oxy)-2-fluorophenyl)-1-((2-(trimethylsilyl)ethoxy)methyl)-1H-imidazole-5-carbonyl)phenyl)propanoate (100 mg, 0.128 mmol) in THF (5 mL) was added (trifluoromethyl)trimethylsilane (183 mg, 1.28 mmol) at 0° C. The reaction mixture was stirred for one hour, potassium tert-butyloxide (72 mg, 0.64 mmol) was added, stirred overnight at room temperature, poured into an ice-cold saturated ammonium chloride solution (20 mL) and extracted with ethyl acetate (30 mL×2). The combined organic extracts were washed with brine (20 mL), dried over sodium sulfate and concentrated to afford the crude title compound as brown syrup (118 mg, 100%). MS-ESI m/z: 922 m/z [M+H]⁺.

### D. Example 273. 3-(3-(1-(2-(5-((4,6-Difluoro-1H-indol-5-yl)oxy)-2-fluorophenyl)-1H-imidazol-5-yl)-2,2,2-trifluoro-1-hydroxyethyl)phenyl)propanoic acid

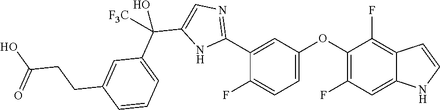 [SMILES: O=C(O)CCc1cccc(C(O)(c2cnc(-c3cc(Oc4c(F)cc5[nH]ccc5c4F)ccc3F)[nH]2)C(F)(F)F)c1]

To a stirred solution of methyl 3-(3-(1-(2-(5-((4,6-difluoro-1-((2-(trimethylsilyl)ethoxy)methyl)-1H-indol-5-yl)oxy)-2-fluorophenyl)-1-((2-(trimethylsilyl)ethoxy)methyl)-1H-imidazol-5-yl)-2,2,2-trifluoro-1-((trimethylsilyl)oxy)ethyl)phenyl)propanoate (100 mg, 0.108 mmol) in THF (10 mL) was added tetrabutylammonium fluoride (1M in THF) (5 mL, 5.0 mmol). The reaction mixture was stirred at 80° C. overnight, diluted with ethyl acetate (80 mL), washed with brine (15 mL), dried over sodium sulfate and concentrated. The resulting residue was purified by flash chromatography (5% methanol in dichloromethane) to give a brown oil, which was further purified by preparative HPLC to afford the title compound as white solid (4.2 mg, 6%). MS-ESI m/z: 576 m/z [M+H]⁺. ¹H NMR (400 MHz, CD₃OD) δ 7.50-7.47 (m, 2H), 7.38-7.36 (m, 1H), 7.31-7.29 (m, 2H), 7.26-7.19 (m, 3H), 7.14 (d, J=10.4 Hz, 1H), 7.02-7.00 (m, 1H), 6.53 (d, J=3.6 Hz, 1H), 2.90 (t, J=7.6 Hz, 2H), 2.57 (t, J=7.6 Hz, 2H) ppm.

### Example 274

### rac-3-(3-((2r,4s)-2-(2-(2-Fluoro-5-((6-fluoro-4-methyl-1H-indol-5-yl)oxy)phenyl)-1H-imidazol-5-yl)-4-hydroxytetrahydrofuran-2-yl)phenyl)propanoic acid

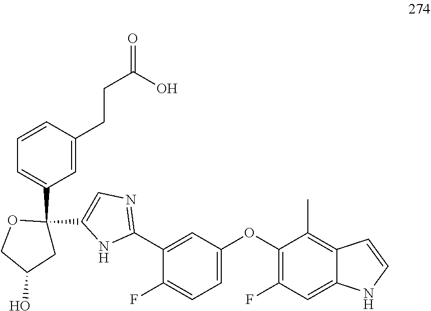 [SMILES: Cc1c(Oc2ccc(F)c(-c3ncc([C@]4(c5cccc(CCC(=O)O)c5)C[C@H](O)CO4)[nH]3)c2)c(F)cc2[nH]ccc12]

### And

### Example 275

### rac-3-(3-((2r,4r)-2-(2-(2-Fluoro-5-((6-fluoro-4-methyl-1H-indol-5-yl)oxy)phenyl)-1H-imidazol-5-yl)-4-hydroxytetrahydrofuran-2-yl)phenyl)propanoic acid

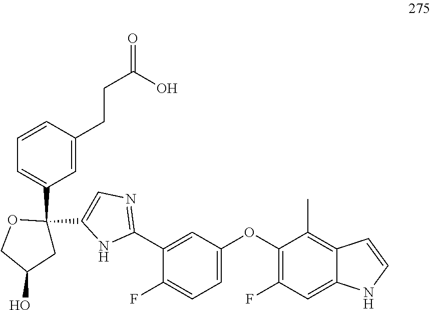 [SMILES: Cc1c(Oc2ccc(F)c(-c3ncc([C@]4(c5cccc(CCC(=O)O)c5)C[C@@H](O)CO4)[nH]3)c2)c(F)cc2[nH]ccc12]

### A. 3-(3-Iodophenyl)acrylaldehyde

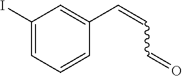 [SMILES: O=CC=Cc1cccc(I)c1]

A mixture of 3-iodobenzaldehyde (2.6 μg, 11.2 mmol) and 2-(triphenyl-□5-phosphaneylidene)acetaldehyde (3.5 g, 11.4 mmol) in toluene (50 mL) was stirred at 90° C. overnight. The mixture was concentrated, diluted with ethyl acetate (200 mL), washed with water (50 mL×2) and brine (50 mL×2), dried over sodium sulfate and concentrated. The resulting residue was purified by flash chromatography over silica (petroleum ether/ethyl acetate, v/v, 6/1) to afford the title compound as an oil (2.2 g, 76%). MS: 259 m/z [M+H]⁺.

### B. 3-(3-Iodophenyl)oxirane-2-carbaldehyde

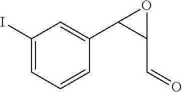 [SMILES: O=CC1OC1c1cccc(I)c1]

To a stirred solution of (Z)-3-(3-Iodophenyl)acrylaldehyde (2.2 g, 8.53 mmol) in methanol (40 mL) was added tert-butyl peroxide (1.9 mL, 10.2 mmol, 5.5 N in decane) dropwise and the pH was maintained at 8.0-8.5 throughout the addition with 1N sodium hydroxide. The reaction mixture was stirred at room temperature overnight, diluted with ethyl acetate (200 mL), washed with water (50 mL×2) and brine (50 mL×2), dried over sodium sulfate and concentrated. The resulting residue was purified by flash chromatography over silica (20% ethyl acetate in petroleum ether) to afford the title compound as an oil (2 g, 86%).

### C. (2-(2-Fluoro-5-((6-fluoro-4-methyl-1H-indol-5-yl)oxy)phenyl)-1H-imidazol-5-yl)(3-iodophenyl)methanol

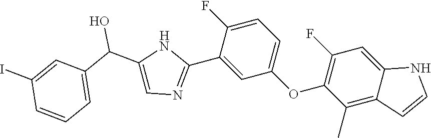 [SMILES: Cc1c(Oc2ccc(F)c(-c3ncc(C(O)c4cccc(I)c4)[nH]3)c2)c(F)cc2[nH]ccc12]

A solution of 3-(3-iodophenyl)oxirane-2-carbaldehyde (1 g, 3.65 mmol) and 2-fluoro-5-((6-fluoro-4-methyl-1H-indol-5-yl)oxy)benzimidamide (989 mg, 3.28 mmol, Intermediate 24-10) in DMF (25 mL) was stirred at 75° C. overnight. The reaction mixture was diluted with ethyl acetate (150 mL), washed with water (50 mL×4) and brine (50 mL×2), dried over sodium sulfate and concentrated. The resulting residue was purified by flash chromatography over silica (petroleum ether/ethyl acetate/dichloromethane, v/v, 2/1/1) to afford the title compound as a light yellow solid (1.1 g, 55%). MS: 558 m/z [M+H]⁺.

### D. (2-(2-Fluoro-5-((6-fluoro-4-methyl-1H-indol-5-yl)oxy)phenyl)-1H-imidazol-5-yl)(3-iodophenyl)methanone

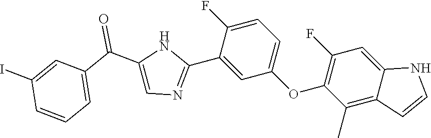 [SMILES: Cc1c(Oc2ccc(F)c(-c3ncc(C(=O)c4cccc(I)c4)[nH]3)c2)c(F)cc2[nH]ccc12]

To a stirred solution of (2-(2-fluoro-5-((6-fluoro-4-methyl-1H-indol-5-yl)oxy)phenyl)-1H-imidazol-5-yl)(3-iodophenyl)methanol (2.2 g, 3.95 mmol) in THF (30 mL) was added manganese dioxide (3.4 g, 39.5 mmol). The reaction mixture was stirred at room temperature overnight and filtered. The filtrate was concentrated and the resulting residue was purified by flash chromatography over silica (petroleum ether/ethyl acetate/dichloromethane, v/v, 2/1/1) to afford the title compound as a light yellow solid (1.7 g, 69%). MS: 556 m/z [M+H]⁺.

### E. 1-(2-(2-Fluoro-5-((6-fluoro-4-methyl-1H-indol-5-yl)oxy)phenyl)-1H-imidazol-4-yl)-1-(3-iodophenyl)but-3-en-1-ol

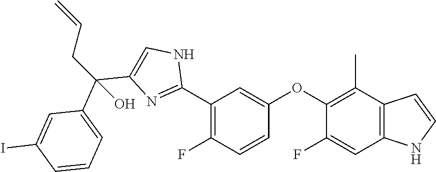 [SMILES: C=CCC(O)(c1cccc(I)c1)c1c[nH]c(-c2cc(Oc3c(F)cc4[nH]ccc4c3C)ccc2F)n1]

To a stirred solution of (2-(2-fluoro-5-((6-fluoro-4-methyl-1H-indol-5-yl)oxy)phenyl)-1H-imidazol-5-yl)(3-iodophenyl)methanone (1.2 g, 2.16 mmol) in THF (20 mL) was added allylmagnesium bromide (1 N in ether, 21.6 mL, 21.6 mmol) at room temperature. The reaction mixture was stirred at room temperature for one hour, diluted with water (20 mL) and extracted with ethyl acetate (100 mL×2). The combined organic extracts were washed with water (50 ml) and brine (50 ml), dried over sodium sulfate, filtered and concentrated. The resulting residue was purified by flash chromatography over silica (petroleum ether/ethyl acetate/dichloromethane, v/v, 2/1/1) to afford the title compound as a light yellow solid (1.2 g, 93%). MS: 598 m/z [M+H]⁺.

### F. 4-(2-(2-Fluoro-5-((6-fluoro-4-methyl-1H-indol-5-yl)oxy)phenyl)-1H-imidazol-4-yl)-4-(3-iodophenyl)butane-1,2,4-triol

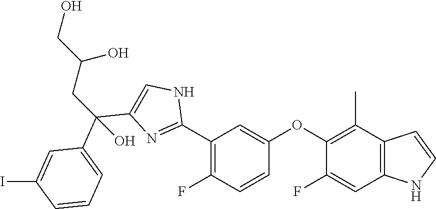 [SMILES: Cc1c(Oc2ccc(F)c(-c3nc(C(O)(CC(O)CO)c4cccc(I)c4)c[nH]3)c2)c(F)cc2[nH]ccc12]

To a stirred solution of 1-(2-(2-fluoro-5-((6-fluoro-4-methyl-1H-indol-5-yl)oxy)phenyl)-1H-imidazol-4-yl)-1-(3-iodophenyl)but-3-en-1-ol (2.4 g, 3.85 mmol) in THF (35 mL) and isopropanol (5 mL) was added 4-methylmorpholine N-oxide (900 mg, 7.71 mmol) and osmium tetroxide (10 drops, 1% in water) at room temperature. The reaction mixture was stirred at room temperature overnight, saturated sodium sulfite (30 mL) was added, stirred for another 0.5 hour and extracted with ethyl acetate (80 mL×2). The combined organic extracts were washed with water (50 ml), brine (50 ml), dried over sodium sulfate, filtered and concentrated. The resulting residue was purified by flash chromatography over silica (10% methanol in dichloromethane) to afford the title compound as a light yellow solid (2.1 g, 86%). MS: 632 m/z [M+H]⁺.

### G. 5-(2-(2-Fluoro-5-((6-fluoro-4-methyl-1H-indol-5-yl)oxy)phenyl)-1H-imidazol-5-yl)-5-(3-iodophenyl)tetrahydrofuran-3-ol

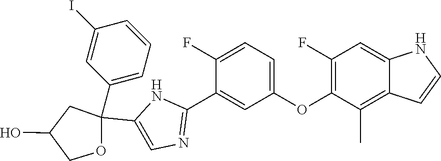 [SMILES: Cc1c(Oc2ccc(F)c(-c3ncc(C4(c5cccc(I)c5)CC(O)CO4)[nH]3)c2)c(F)cc2[nH]ccc12]

To a stirred solution of 4-(2-(2-fluoro-5-((6-fluoro-4-methyl-1H-indol-5-yl)oxy)phenyl)-1H-imidazol-4-yl)-4-(3-iodophenyl)butane-1,2,4-triol (1.1 g, 1.74 mmol) in dichloromethane (30 mL) was added p-toluenesulfonic acid (60 mg, 0.35 mmol). The reaction mixture was stirred at 50° C. overnight, diluted with water (40 mL) and extracted with ethyl acetate (70 mL×3). The combined organic extracts were washed with water (50 ml) and brine (50 ml), dried over sodium sulfate, filtered and concentrated. The resulting residue was purified by flash chromatography over silica (petroleum ether/ethyl acetate/dichloromethane, v/v, 2/1/1) to afford the title compound as light yellow solid (640 mg, 60%). MS: 614 m/z [M+H]⁺.

### H. rac-Ethyl-(E)-3-(3-((2r,4s)-2-(2-(2-fluoro-5-((6-fluoro-4-methyl-1H-indol-5-yl)oxy)phenyl)-1H-imidazol-5-yl)-4-hydroxytetrahydrofuran-2-yl)phenyl)acrylate (Isomer 1) and rac-ethyl (E)-3-(3-((2r,4r)-2-(2-(2-fluoro-5-((6-fluoro-4-methyl-1H-indol-5-yl)oxy)phenyl)-1H-imidazol-5-yl)-4-hydroxytetrahydrofuran-2-yl)phenyl)acrylate (Isomer 2) (Relative Stereochemistry Arbitrarily Assigned)

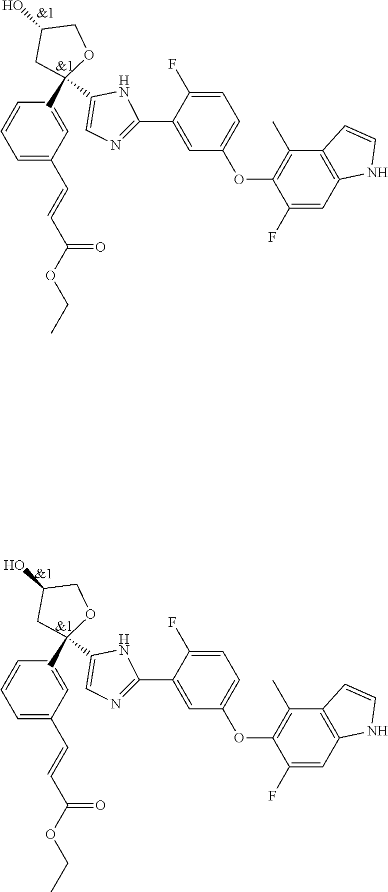

A solution of 5-(2-(2-fluoro-5-((6-fluoro-4-methyl-1H-indol-5-yl)oxy)phenyl)-1H-imidazol-5-yl)-5-(3-iodophenyl)tetrahydrofuran-3-ol (620 mg, 1.01 mmol), ethyl acrylate (303 mg, 3.03 mmol), palladium(II) acetate (23 mg, 0.10 mmol), P(o-Tol)₃ (92 mg, 0.30 mmol) and triethylamine (306 mg, 3.03 mmol) in DMF (15 mL) was heated in a microwave reactor at 110° C. for 0.5 hours. The reaction mixture was diluted with ethyl acetate (100 mL), washed with water (50 ml×4) and brine (50 ml×2), dried over sodium sulfate, filtered and concentrated. The resulting residue was purified by flash chromatography over silica (10% methanol in dichloromethane) and then preparative HPLC to give rac-ethyl-(E)-3-(3-((2r,4s)-2-(2-(2-fluoro-5-((6-fluoro-4-methyl-1H-indol-5-yl)oxy)phenyl)-1H-imidazol-5-yl)-4-hydroxytetrahydrofuran-2-yl)phenyl)acrylate (32 mg, Isomer 1) and rac-ethyl-(E)-3-(3-((2r,4r)-2-(2-(2-fluoro-5-((6-fluoro-4-methyl-1H-indol-5-yl)oxy)phenyl)-1H-imidazol-5-yl)-4-hydroxytetrahydrofuran-2-yl)phenyl)acrylate (78 mg, Isomer 2) as cis and trans racemic isomers (hydroxyl relative to imidazole ring, the relative stereochemistries were arbitrarily assigned), both as white solid. Total yield: 19%. MS: 586 m/z [M+H]⁺.

### I. rac-Ethyl 3-(3-((2r,4s)-2-(2-(2-fluoro-5-((6-fluoro-4-methyl-1H-indol-5-yl)oxy)phenyl)-1H-imidazol-5-yl)-4-hydroxytetrahydrofuran-2-yl)phenyl)propanoate

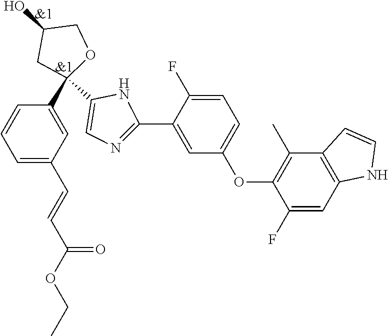 [SMILES: CCOC(=O)/C=C/c1cccc([C@@]2(c3cnc(-c4cc(Oc5c(F)cc6[nH]ccc6c5C)ccc4F)[nH]3)C[C@@H](O)CO2)c1]

To a stirred solution of rac-ethyl (E)-3-(3-((2r,4s)-2-(2-(2-fluoro-5-((6-fluoro-4-methyl-1H-indol-5-yl)oxy)phenyl)-1H-imidazol-5-yl)-4-hydroxytetrahydrofuran-2-yl)phenyl)acrylate (Isomer 1) (47 mg, 0.08 mmol) in THF (7 mL) was added 10% Pd/C (30 mg) and the mixture was stirred at room temperature under hydrogen for five hours. The mixture was filtered and the filtrate was concentrated to afford the title compound as a light yellow solid (40 mg, 85%, Isomer 1). MS: 588 m/z [M+H]⁺.

### J. Example 274. rac-3-(3-((2r,4s)-2-(2-(2-Fluoro-5-((6-fluoro-4-methyl-1H-indol-5-yl)oxy)phenyl)-1H-imidazol-5-yl)-4-hydroxytetrahydrofuran-2-yl)phenyl)propanoic acid

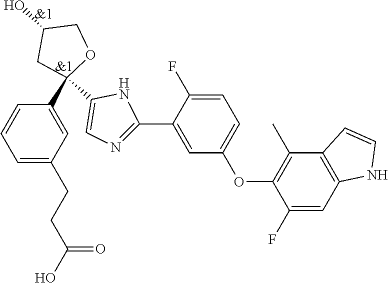 [SMILES: Cc1c(Oc2ccc(F)c(-c3ncc([C@]4(c5cccc(CCC(=O)O)c5)C[C@H](O)CO4)[nH]3)c2)c(F)cc2[nH]ccc12]

To a stirred solution of rac-ethyl 3-(3-((2r,4s)-2-(2-(2-fluoro-5-((6-fluoro-4-methyl-1H-indol-5-yl)oxy)phenyl)-1H-imidazol-5-yl)-4-hydroxytetrahydrofuran-2-yl)phenyl)propanoate (40 mg, 0.07 mmol, Isomer 1) in THF (5 mL) was added lithium hydroxide (11 mg, 0.42 mmol) in water (0.5 mL). The reaction mixture was stirred at room temperature overnight, diluted with water (10 mL), acidified with 1N hydrochloric acid to pH˜4 and extracted with ethyl acetate (30 mL×3). The combined organic extracts were concentrated and the resulting residue was purified by preparative HPLC to afford the title compound as a white solid (10.5 mg, 27%, Isomer 1). MS: 560 m/z [M+H]⁺. ¹H NMR (400 MHz, CD₃OD) δ 7.46-7.43 (m, 1H), 7.40 (s, 1H), 7.32-7.30 (m, 1H), 7.27 (d, J=3.2 Hz, 1H), 7.24-7.20 (m, 1H), 7.16-7.10 (m, 3H), 6.85 (s, 1H), 6.84-6.81 (m, 1H), 6.51 (d, J=2.4 Hz, 1H), 4.63-4.60 (m, 1H), 4.11-4.08 (m, 1H), 3.87 (dd, J=2.8, 9.2 Hz, 1H), 3.19-3.14 (m, 1H), 2.91 (t, J=8.0 Hz, 2H), 2.57 (t, J=8.0 Hz, 2H), 2.39 (s, 3H), 2.33 (dd, J=4.4, 13.6 Hz, 1H) ppm.

### K. Example 275. rac-3-(3-((2r,4r)-2-(2-(2-Fluoro-5-((6-fluoro-4-methyl-1H-indol-5-yl)oxy)phenyl)-1H-imidazol-5-yl)-4-hydroxytetrahydrofuran-2-yl)phenyl)propanoic acid

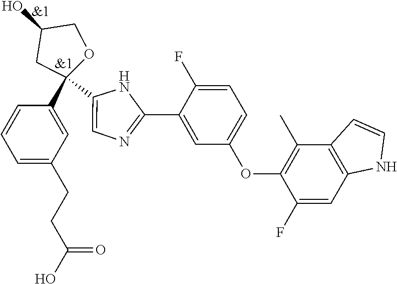 [SMILES: Cc1c(Oc2ccc(F)c(-c3ncc([C@]4(c5cccc(CCC(=O)O)c5)C[C@@H](O)CO4)[nH]3)c2)c(F)cc2[nH]ccc12]

Using the conditions described in steps I and J for Example 274 above but exchanging rac-ethyl (E)-3-(3-((2r,4s)-2-(2-(2-fluoro-5-((6-fluoro-4-methyl-1H-indol-5-yl)oxy)phenyl)-1H-imidazol-5-yl)-4-hydroxytetrahydrofuran-2-yl)phenyl)acrylate (Isomer 1) for rac-ethyl (E)-3-(3-((2r,4r)-2-(2-(2-fluoro-5-((6-fluoro-4-methyl-1H-indol-5-yl)oxy)phenyl)-1H-imidazol-5-yl)-4-hydroxytetrahydrofuran-2-yl)phenyl)acrylate (Isomer 2, relative stereochemistry randomly assigned), the title compound was obtained as a white solid (16 mg). MS: 560 m/z [M+H]⁺. ¹H NMR (400 MHz, CD₃OD) δ 7.51-7.48 (m, 1H), 7.39 (s, 1H), 7.34-7.27 (m, 3H), 7.22-7.11 (m, 3H), 6.81-6.77 (m, 1H), 6.52 (d, J=2.4 Hz, 1H), 6.31 (s, 1H), 4.54-4.52 (m, 1H), 4.10-4.08 (m, 1H), 3.99 (dd, J=3.6, 9.2 Hz, 1H), 2.95 (t, J=7.6 Hz, 2H), 2.87-2.83 (m, 1H), 2.60 (dd, J=4.2, 13.6 Hz, 1H), 2.51 (t, J=8.0 Hz, 2H), 2.40 (s, 3H) ppm.

### Example 276

### 3-(3-(1,1-Difluoro-2-(2-(2-fluoro-5-((6-fluoro-4-(methylsulfonyl)-1H-indol-5-yl)oxy)phenyl)-1H-imidazol-5-yl)propan-2-yl)phenyl)propanoic acid

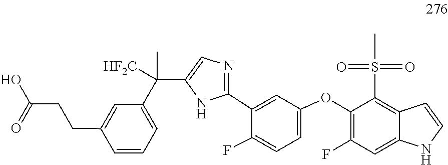

### A. 5-(3-(5-(1,1-Difluoro-2-(3-iodophenyl)propan-2-yl)-1H-imidazol-2-yl)-4-fluorophenoxy)-6-fluoro-4-(methylthio)-1H-indole

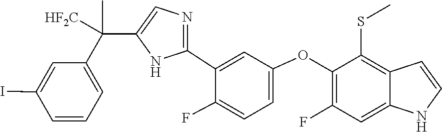

Exchanging methyl 3-(3-(3-bromo-2-oxopropyl)phenyl)propanoate (Intermediate 21) for 1-bromo-4,4-difluoro-3-(3-iodophenyl)-3-methylbutan-2-one (332 mg, 0.83 mmol, Intermediate 38), the reaction procedure described in step A of Example 22 was used to prepare the title compound as a solid (244 mg, 51%). MS: 638 m/z [M+H]⁺.

### B. 5-(3-(5-(1,1-Difluoro-2-(3-iodophenyl)propan-2-yl)-1H-imidazol-2-yl)-4-fluorophenoxy)-6-fluoro-4-(methylsulfonyl)-1H-indole

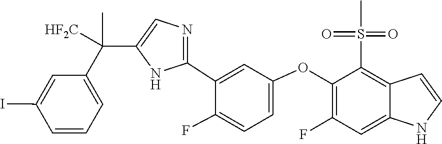

Exchanging methyl 3-(3-((2-(2-fluoro-5-((6-fluoro-4-(methylthio)-1H-indol-5-yl)oxy)phenyl)-1H-imidazol-5-yl)methyl)phenyl)propanoate for 5-(3-(5-(1,1-Difluoro-2-(3-iodophenyl)propan-2-yl)-1H-imidazol-2-yl)-4-fluorophenoxy)-6-fluoro-4-(methylthio)-1H-indole (244 mg, 0.06 mmol), the reaction procedure described in step B of Example 22 was used to prepare the title compound as a solid (120 mg, 47%). MS: 670 m/z [M+H]⁺.

### C. Example 276. 3-(3-(1,1-Difluoro-2-(2-(2-fluoro-5-((6-fluoro-4-(methylsulfonyl)-1H-indol-5-yl)oxy)phenyl)-1H-imidazol-5-yl)propan-2-yl)phenyl)propanoic acid

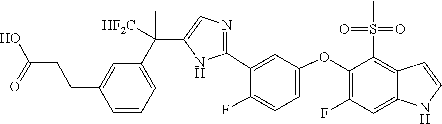

Exchanging (2-(3-((6-fluoro-4-(methylsulfonyl)-1H-indol-5-yl)oxy)phenyl)thiazol-4-yl)(3-iodophenyl)methanone for 5-(3-(5-(1,1-Difluoro-2-(3-iodophenyl)propan-2-yl)-1H-imidazol-2-yl)-4-fluorophenoxy)-6-fluoro-4-(methylsulfonyl)-1H-indole (120 mg, 0.18 mmol), the two step reaction sequence (Steps C and D) described for Example 37 was used to prepare the title compound as a solid (18 mg, 15% from two steps). MS: 616 m/z [M+H]⁺. ¹H NMR (400 MHz, CD₃OD) δ 7.66-7.51 (m, 3H), 7.22-7.11 (m, 7H), 6.94-6.92 (m, 1H), 6.53-6.25 (t, 1H), 3.32 (s, 3H), 2.89-2.85 (t, 2H), 2.51-2.47 (t, 2H), 1.79 (s, 3H) ppm.

### Example 277

### 3-(3-(1-(2-(5-((4,6-difluoro-1H-indol-5-yl)oxy)-2-fluorophenyl)-1H-imidazol-5-yl)-2,2-dimethylpropyl)phenyl)propanoic acid

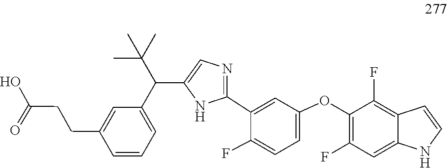 [SMILES: CC(C)(C)C(c1cccc(CCC(=O)O)c1)c1cnc(-c2cc(Oc3c(F)cc4[nH]ccc4c3F)ccc2F)[nH]1]

### A. 1-(2-(5-((4,6-Difluoro-1H-indol-5-yl)oxy)-2-fluorophenyl)-1H-imidazol-5-yl)-1-(3-iodophenyl)-2,2-dimethylpropan-1-ol

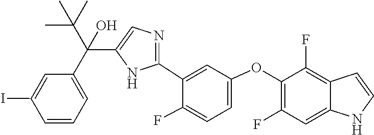 [SMILES: CC(C)(C)C(O)(c1cccc(I)c1)c1cnc(-c2cc(Oc3c(F)cc4[nH]ccc4c3F)ccc2F)[nH]1]

Exchanging 2-fluoro-5-((6-fluoro-4-methyl-1H-indol-5-yl)oxy)benzimidamide (Intermediate 24-10) for 5-((4,6-difluoro-1H-indol-5-yl)oxy)-2-fluorobenzimidamide (Intermediate 24-9) and allylmagnesium bromide for tert-Butyl magnesium chloride, the three step reaction sequence described for Example 274 and Example 275 (Steps C to E) was used to prepare the title compound as a white solid (570 mg, 6%). MS-ESI m/z: 618 m/z [M+H]⁺.

### B. Methyl (E)-3-(3-(1-(2-(5-((4,6-difluoro-1H-indol-5-yl)oxy)-2-fluorophenyl)-1H-imidazol-5-yl)-1-hydroxy-2,2-dimethylpropyl)phenyl)acrylate

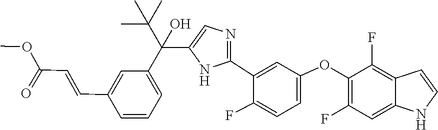 [SMILES: COC(=O)/C=C/c1cccc(C(O)(c2cnc(-c3cc(Oc4c(F)cc5[nH]ccc5c4F)ccc3F)[nH]2)C(C)(C)C)c1]

Exchanging 3-iodophenylacetic acid for 1-(2-(5-((4,6-difluoro-1H-indol-5-yl)oxy)-2-fluorophenyl)-1H-imidazol-5-yl)-1-(3-iodophenyl)-2,2-dimethylpropan-1-ol, the Heck coupling reaction procedure described for Intermediate 21A was used to prepare the title compound as a yellow solid. MS-ESI m/z: 576 m/z [M+H]⁺.

### C. Methyl 3-(3-(1-(2-(5-((4,6-difluoro-1H-indol-5-yl)oxy)-2-fluorophenyl)-1H-imidazol-5-yl)-2,2-dimethylpropyl)phenyl)propanoate

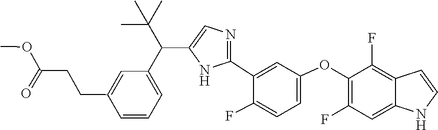 [SMILES: COC(=O)CCc1cccc(C(c2cnc(-c3cc(Oc4c(F)cc5[nH]ccc5c4F)ccc3F)[nH]2)C(C)(C)C)c1]

To a stirred solution of methyl (E)-3-(3-(1-(2-(5-((4,6-difluoro-1H-indol-5-yl)oxy)-2-fluorophenyl)-1H-imidazol-5-yl)-1-hydroxy-2,2-dimethylpropyl)phenyl)acrylate (230 mg, 0.45 mmol) in acetic acid (6 mL) was added Pd/C (10% on carbon) (200 mg) and ammonium formate (712 mg, 11.3 mmol). The mixture was degassed with nitrogen and stirred at 110° C. for two hours. The mixture was filtered. evaporated and purified by preparative TLC (50% ethyl acetate in petroleum ether) to afford the title compound as a white solid (160 mg, 71%). MS-ESI m/z: 562 m/z [M+H]⁺.

### D. Example 277. 3-(3-(1-(2-(5-((4,6-Difluoro-1H-indol-5-yl)oxy)-2-fluorophenyl)-1H-imidazol-5-yl)-2,2-dimethylpropyl)phenyl)propanoic acid

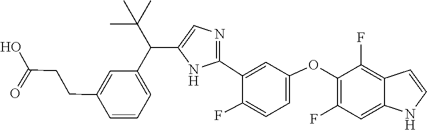 [SMILES: CC(C)(C)C(c1cccc(CCC(=O)O)c1)c1cnc(-c2cc(Oc3c(F)cc4[nH]ccc4c3F)ccc2F)[nH]1]

A solution of methyl 3-(3-(1-(2-(5-((4,6-difluoro-1H-indol-5-yl)oxy)-2-fluorophenyl)-1H-imidazol-5-yl)-2,2-dimethylpropyl)phenyl)propanoate (160 mg, 0.29 mmol) and lithium hydroxide (68 mg, 2.9 mmol) in THF (6 mL) and water (3 mL) was stirred at room temperature for three hours. Most of THF was removed under reduced pressure and the residue was acidified with 3M hydrochloric acid to pH to ˜5. The precipitate was collected by filtration and dried to afford the title compound as a white solid (148.4 mg, 95%). ¹H NMR (400 MHz, CD₃OD) δ 7.59 (s, 1H), 7.50-7.48 (m, 1H), 7.33-7.22 (m, 5H), 7.17-7.07 (m, 3H), 6.57 (d, J=3.2 Hz, 1H), 3.96 (s, 1H), 2.92 (t, J=7.6 Hz, 2H), 2.60 (t, J=7.6 Hz, 2H), 1.01 (s, 9H) ppm. MS-ESI m/z: 548 m/z [M+H]⁺.

### Example 278

### Enantiomer 1 of 3-(3-(1-(2-(5-((4,6-difluoro-1H-indol-5-yl)oxy)-2-fluorophenyl)-1-methyl-1H-imidazol-4-yl)ethyl)-2-fluorophenyl)propanoic acid (278A)

### And

### Enantiomer 2 of 3-(3-(1-(2-(5-((4,6-difluoro-1H-indol-5-yl)oxy)-2-fluorophenyl)-1-methyl-1H-imidazol-4-yl)ethyl)-2-fluorophenyl)propanoic acid (278B)

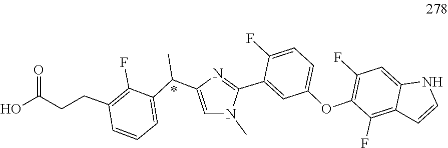 [SMILES: CC(c1cn(C)c(-c2cc(Oc3c(F)cc4[nH]ccc4c3F)ccc2F)n1)c1cccc(CCC(=O)O)c1F]

### A. 5-((4,6-Difluoro-1H-indol-5-yl)oxy)-2-fluoro-N-methylbenzimidamide

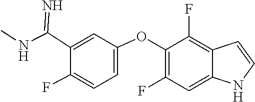 [SMILES: CNC(=N)c1cc(Oc2c(F)cc3[nH]ccc3c2F)ccc1F]

To a stirred solution of methylamine hydrochloride (0.469 g, 6.94 mmol) in toluene (10 mL) was added trimethylaluminium (6.94 mL, 6.94 mmol) at 0° C. under argon. The reaction mixture was stirred at room temperature for two hours, 5-[(4,6-difluoro-1H-indol-5-yl)oxy]-2-fluorobenzonitrile (100 mg, 0.347 mmol, Product of Step H, Example 100) was added and stirred at 120° C. overnight. The reaction mixture was cooled to room temperature, diluted with water (20 mL) and extracted with ethyl acetate (20 mL×3). The combined organic extracts were washed with brine, dried over sodium sulfate and concentrated. The resulting crude product was purified by flash chromatography over silica (6% methanol in dichloromethane) to afford the title compound as a yellow solid (70 mg, 63%). MS: 320 m/z [M+H]⁺.

### B. Ethyl 3-(3-(1-(2-(5-((4,6-difluoro-1H-indol-5-yl)oxy)-2-fluorophenyl)-1-methyl-1H-imidazol-4-yl)ethyl)-2-fluorophenyl)propanoate

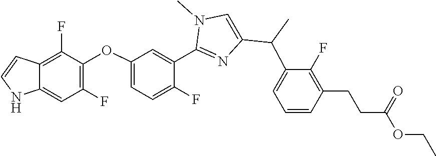 [SMILES: CCOC(=O)CCc1cccc(C(C)c2cn(C)c(-c3cc(Oc4c(F)cc5[nH]ccc5c4F)ccc3F)n2)c1F]

To a stirred solution of ethyl 3-(3-(4-bromo-3-oxobutan-2-yl)-2-fluorophenyl)propanoate (280 mg, 0.811 mmol Intermediate 21-9) in DMF (10 mL) was added 5-((4,6-Difluoro-1H-indol-5-yl)oxy)-2-fluoro-N-methylbenzimidamide (259 mg, 0.811 mmol) and sodium bicarbonate (136 mg, 1.62 mmol) under nitrogen. The mixture was stirred at 75° C. overnight, cooled to room temperature, water (30 mL) was added and extracted with ethyl acetate (40 mL×3). The combined organic extracts were washed with brine, dried over sodium sulfate and concentrated. The resulting residue was purified by preparative HPLC to afford the title compound as a white solid (150 mg, 33%). MS: 566 m/z [M+H]⁺.

### C. Chiral Separation

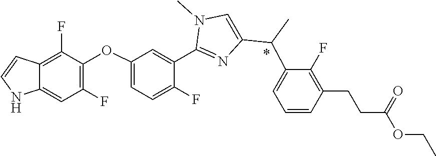 [SMILES: CCOC(=O)CCc1cccc(C(C)c2cn(C)c(-c3cc(Oc4c(F)cc5[nH]ccc5c4F)ccc3F)n2)c1F]

Racemic ethyl 3-(3-(1-(2-(5-((4,6-difluoro-1H-indol-5-yl)oxy)-2-fluorophenyl)-1-methyl-1H-imidazol-4-yl)ethyl)-2-fluorophenyl)propanoate (150 mg, 0.265 mmol) was separated by SFC using a (S,S)WHELK column (20×250 mm, 10 micron), eluting with 40% carbon dioxide and 60% methanol (with 0.2% ammonia saturated in methanol), at a back pressure of 100 par, flow rate of 80 g/min and column temperature of 35° C.

Peak 1: Pure enantiomer 1 of ethyl 3-(3-(1-(2-(5-((4,6-difluoro-1H-indol-5-yl)oxy)-2-fluorophenyl)-1-methyl-1H-imidazol-4-yl)ethyl)-2-fluorophenyl)propanoate as a white solid (51 mg, 34%).

Peak 2: Pure enantiomer 2 of ethyl 3-(3-(1-(2-(5-((4,6-difluoro-1H-indol-5-yl)oxy)-2-fluorophenyl)-1-methyl-1H-imidazol-4-yl)ethyl)-2-fluorophenyl)propanoate as a white solid (48 mg, 32%).

### D. Example 278A. Enantiomer 1 of 3-(3-(1-(2-(5-((4,6-difluoro-1H-indol-5-yl)oxy)-2-fluorophenyl)-1-methyl-1H-imidazol-4-yl)ethyl)-2-fluorophenyl)propanoic acid

To a stirred solution of enantiomer 1 of ethyl 3-(3-(1-(2-(5-((4,6-difluoro-1H-indol-5-yl)oxy)-2-fluorophenyl)-1-methyl-1H-imidazol-4-yl)ethyl)-2-fluorophenyl)propanoate (51 mg, 0.09 mmol) in a mixture of methanol (1 mL), THF (2 mL) and water (1 mL) was added lithium hydroxide (18.9 mg, 0.45 mmol). The reaction mixture was stirred at room temperature for two hours, water (10 mL) was added and THF was removed under reduced pressure. The residue was dissolved in water (1 mL), and the pH was adjusted to ˜4 with hydrochloric acid (1N). The precipitate was collected by filtration to give the title compound as a yellow solid (41.7 mg, 86%). MS: 538 m/z [M+H]⁺. LC-MS retention time: 0.96 min.

### E. Example 278B. Enantiomer 2 of 3-(3-(1-(2-(5-((4,6-difluoro-1H-indol-5-yl)oxy)-2-fluorophenyl)-1-methyl-1H-imidazol-4-yl)ethyl)-2-fluorophenyl)propanoic acid

Exchanging enantiomer 1 of ethyl 3-(3-(1-(2-(5-((4,6-difluoro-1H-indol-5-yl)oxy)-2-fluorophenyl)-1-methyl-1H-imidazol-4-yl)ethyl)-2-fluorophenyl)propanoate for enantiomer 2, the hydrolysis reaction procedure described for the synthesis of Example 278A above, was used to prepare the title compound as a yellow solid (39.4 mg, 86%). MS: 538 m/z [M+H]⁺. ¹H NMR (400 MHz, CD₃OD) δ 7.56 (s, 1H), 7.42 (t, J=9.2 Hz, 1H), 7.34-7.23 (m, 4H), 7.20-7.01 (m, 3H), 6.56 (d, J=2.7 Hz, 1H), 4.65-4.47 (m, 1H), 3.80 (s, 3H), 2.97 (t, J=7.4 Hz, 2H), 2.61 (t, J=7.6 Hz, 2H), 1.70 (d, J=7.1 Hz, 3H) ppm. LC-MS retention time: 0.96 min.

### Example 279

### 3-(2-Fluoro-3-((2-(2-fluoro-5-((6-fluoro-4-((methylsulfonyl)methyl)-1H-indol-5-yl)oxy)phenyl)-1H-imidazol-5-yl)methyl)phenyl)propanoic acid

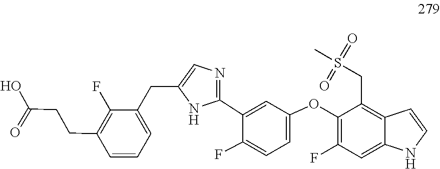 [SMILES: CS(=O)(=O)Cc1c(Oc2ccc(F)c(-c3ncc(Cc4cccc(CCC(=O)O)c4F)[nH]3)c2)c(F)cc2[nH]ccc12]

### A. 2-Fluoro-5-((6-fluoro-4-((methylsulfonyl)methyl)-1-(phenylsulfonyl)-1H-indol-5-yl)oxy)benzonitrile

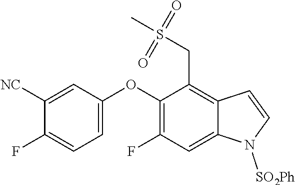

A mixture of 5-((4-(bromomethyl)-6-fluoro-1-(phenylsulfonyl)-1H-indol-5-yl)oxy)-2-fluorobenzonitrile (0.502 g, 1.0 mmol, Intermediate 42) and methanesulfinic acid, sodium salt (0.51 g, 5.0 mmol) in DMF (5 mL) was stirred at room temperature overnight. The mixture was quenched with water (10 mL) and extracted with ethyl acetate (10 mL×3). The combined organic phase was washed with brine (20 mL), dried over sodium sulfate, filtered and concentrated. The resulting residue was purified by flash chromatography (15-25% ethyl acetate in petroleum ether) to afford the title compound as a yellow oil (0.4 g, 80%). MS: 525 m/z [M+Na]⁺.

### B. 2-Fluoro-5-((6-fluoro-4-((methylsulfonyl)methyl)-1-(phenylsulfonyl)-1H-indol-5-yl)oxy)benzimidamide

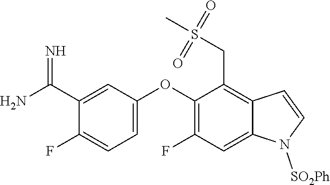

Exchanging 5-(benzo[d]thiazol-5-yloxy)-2-fluorobenzonitrile for 2-fluoro-5-((6-fluoro-4-((methylsulfonyl)methyl)-1-(phenylsulfonyl)-1H-indol-5-yl)oxy)benzonitrile (0.4 μg, 0.8 mmol), the procedure described for Intermediate 43 was used to prepare the title compound. MS: 520 m/z [M+H]⁺.

### C. Ethyl 3-(2-Fluoro-3-((2-(2-fluoro-5-((6-fluoro-4-((methylsulfonyl)methyl)-1-(phenylsulfonyl)-1H-indol-5-yl)oxy)phenyl)-1H-imidazol-5-yl)methyl)phenyl)propanoate

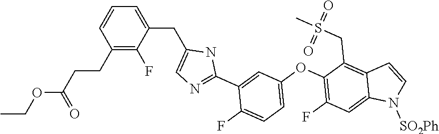

Exchanging 5-((4,6-difluoro-1H-indol-5-yl)oxy)-2-fluorobenzimidamide for 2-fluoro-5-((6-fluoro-4-((methylsulfonyl)methyl)-1-(phenylsulfonyl)-1H-indol-5-yl)oxy)benzimidamide (0.4 g, 0.8 mmol), the procedure described in Step J of Example 100 was used to prepare the title compound. MS: 752 m/z [M+H]⁺.

### D. Example 279. 3-(2-Fluoro-3-((2-(2-fluoro-5-((6-fluoro-4-((methylsulfonyl)methyl)-1H-indol-5-yl)oxy)phenyl)-1H-imidazol-5-yl)methyl)phenyl)propanoic acid

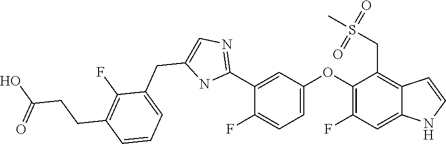

To a stirred solution of ethyl 3-(2-fluoro-3-((2-(2-fluoro-5-((6-fluoro-4-((methylsulfonyl)methyl)-1-(phenylsulfonyl)-1H-indol-5-yl)oxy)phenyl)-1H-imidazol-5-yl)methyl)phenyl)propanoate (100 mg, 0.14 mmol) in THF (3 mL) was added lithium hydroxide (30 mg, 0.7 mmol) in water (1 mL). The reaction mixture was stirred at room temperature overnight and then diluted with water (10 mL). The pH of the mixture was adjusted to ˜6 with 1N hydrochloric acid and extracted with ethyl acetate (30 mL×3). The combined organic extracts were washed with water and brine, dried over sodium sulfate, filtered and concentrated. The crude product was purified by preparative HPLC to give the title compound as a white solid (36.5 mg, 47%). MS: 584 m/z [M+H]⁺. ¹H NMR (400 MHz, CD₃OD) δ 7.46 (dd, J=6.0, 3.2 Hz, 1H), 7.40 (d, J=3.2 Hz, 1H), 7.37 (d, J=10.8 Hz, 1H), 7.25-7.12 (m, 2H), 7.09-7.05 (m, 1H), 7.04-6.95 (m, 2H), 6.77 (s, 1H), 6.72 (d, J=3.2 Hz, 1H), 4.69 (s, 2H), 3.97 (s, 2H), 3.04-2.87 (m, 5H), 2.58 (t, J=7.6 Hz, 2H) ppm.

### Example 280

### 3-(3-((2-(5-((4,6-Difluoro-1H-indol-5-yl)oxy)-2-fluorophenyl)-4-(2-hydroxypropan-2-yl)-1H-imidazol-5-yl)methyl)-2-fluorophenyl)propanoic acid

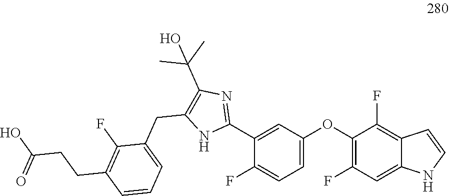 [SMILES: CC(C)(O)c1nc(-c2cc(Oc3c(F)cc4[nH]ccc4c3F)ccc2F)[nH]c1Cc1cccc(CCC(=O)O)c1F]

### A. (5-((4,6-Difluoro-1H-indol-5-yl)oxy)-2-fluorophenyl)methanamine

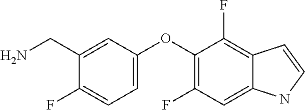

To a stirred solution of 5-((4,6-difluoro-1H-indol-5-yl)oxy)-2-fluorobenzonitrile (10 g, 347 mmol) in THF (200 mL) were added saturated aqueous ammonium hydroxide (20 mL) and Raney Nickel (10 g). The mixture was stirred at room temperature under a hydrogen balloon overnight and filtered through a pad of Celite. The filtrate was concentrated and the residue was purified by flash chromatography over silica gel (10% methanol in dichloromethane) to afford the title compound as a yellow solid. MS: 293 m/z [M+H]⁺.

### B. Methyl 4-(3-bromo-2-fluorophenyl)-3-oxobutanoate

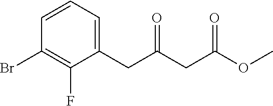 [SMILES: COC(=O)CC(=O)Cc1cccc(Br)c1F]

To a stirred solution of potassium 3-ethoxy-3-oxo-propanoate (15.3 g, 90.1 mmol) in acetonitrile (200 mL) were added magnesium dichloride monohydrate (10.2 g, 107 mmol) and N,N-diethylethanamine (13.9 g, 137 mmol) and the mixture was stirred at room temperature for two hours (solution A). To another solution of 2-(3-bromo-2-fluoro-phenyl)acetic acid (10 g, 43 mmol) in acetonitrile (100 mL) was added carbonyldiimidazole (7.65 g, 47.2 mmol) at room temperature and the mixture was stirred for 1.5 hours. The above solution A was added to this mixture, stirred for 30 minutes and then heated to 80° C. for two hours. The reaction mixture was cooled to room temperature, acidified with hydrochloric acid (2N, 160 mL) and extracted with ethyl acetate. The combined organic extracts were washed with saturated sodium hydrogen carbonate and brine, dried over sodium sulfate, filtered and concentrated. The residue was purified by column chromatography over silica gel (0-15% ethyl acetate in petroleum ether) to afford the title compound as a liquid (10 g, 81%). MS: 288, 290 m/z [M+H]⁺.

### C. Methyl 4-(3-bromo-2-fluorophenyl)-2-(hydroxyimino)-3-oxobutanoate

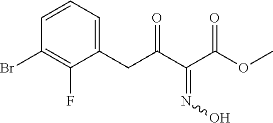 [SMILES: COC(=O)C(=NO)C(=O)Cc1cccc(Br)c1F]

To a stirred solution of methyl 4-(3-bromo-2-fluoro-phenyl)-3-oxo-butanoate (10 g, 33.2 mmol) in acetic acid (120 mL) was added sodium nitrite (2.41 g, 34.9 mmol) in water (60 mL) at 0° C. The mixture was stirred for three hours and extracted with ethyl acetate (100 mL×4). The combined organic phase was washed with brine, dried over sodium sulfate, filtered and concentrated. The residue was purified by column chromatography over silica gel (0-30% ethyl acetate in petroleum ether) to afford the title compound as a yellow oil (7.7 g, 68%).

### D. Methyl 5-(3-bromo-2-fluorobenzyl)-2-(5-((4,6-difluoro-1H-indol-5-yl)oxy)-2-fluorophenyl)-1H-imidazole-4-carboxylate

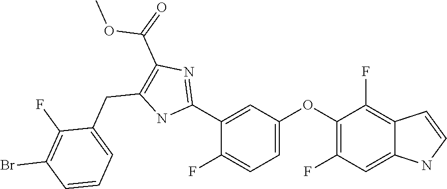

A solution of methyl 4-(3-bromo-2-fluoro-phenyl)-2-hydroxyimino-3-oxo-butanoate (4.35 g, 13.0 mmol) and [5-[(4,6-difluoro-1H-indol-5-yl)oxy]-2-fluoro-phenyl]methanamine (3.96 g, 13.0 mmol) in acetonitrile (170 mL) was refluxed overnight and then concentrated. The residue was purified by flash chromatography over silica (0-50% ethyl acetate in petroleum ether) to afford the title compound as a yellow oil (2.7 g, 31%). MS: 574, 576 m/z [M+H]⁺.

### E. 3-(3-((2-(5-((4,6-Difluoro-1H-indol-5-yl)oxy)-2-fluorophenyl)-4-(methoxycarbonyl)-1H-imidazol-5-yl)methyl)-2-fluorophenyl)propanoic acid

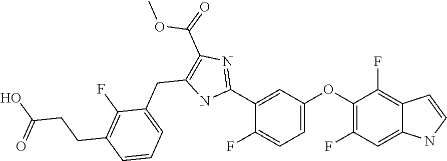

Exchanging (5-(3-(4-(3-iodobenzyl)thiazol-2-yl)phenoxy)-1H-indol-4-yl)methanol for methyl 5-(3-bromo-2-fluorobenzyl)-2-(5-((4,6-difluoro-1H-indol-5-yl)oxy)-2-fluorophenyl)-1H-imidazole-4-carboxylate, the three step reaction sequence described for Example 99 (Steps D, E, F) was used to prepare the title compound as a yellow solid. MS: 568 m/z [M+H]⁺.

### F. Example 280. 3-(3-((2-(5-((4,6-Difluoro-1H-indol-5-yl)oxy)-2-fluorophenyl)-4-(2-hydroxypropan-2-yl)-1H-imidazol-5-yl)methyl)-2-fluorophenyl)propanoic acid

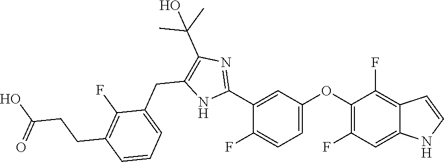 [SMILES: CC(C)(O)c1nc(-c2cc(Oc3c(F)cc4[nH]ccc4c3F)ccc2F)[nH]c1Cc1cccc(CCC(=O)O)c1F]

To a stirred solution of 3-[3-[[2-[5-[(4,6-difluoro-1H-indol-5-yl)oxy]-2-fluoro-phenyl]-4-methoxycarbonyl-1H-imidazol-5-yl]methyl]-2-fluoro-phenyl]propanoic acid (400 mg, 0.53 mmol) in THF (5 mL) was added methylmagnesium bromide (5 mL, 15 mmol, 3 M in diethyl ether) dropwise at 0° C. The reaction mixture was stirred at 0° C. for four hours, quenched with water (20 mL), the pH was adjusted to ˜4 with 1N hydrochloric acid and extracted with ethyl acetate (30 mL×3). The combined organic extracts were washed with brine, dried over sodium sulfate, filtered, and concentrated. The crude product was purified by preparative HPLC to afford the title compound (8.2 mg, 2.65%). MS: 568 m/z [M+H]⁺. ¹H NMR (400 MHz, CD₃OD) δ 7.37-7.31 (m, 1H), 7.18 (d, J=3.2 Hz, 1H), 7.10-6.96 (m, 3H), 6.87-6.80 (m, 2H), 6.74 (t, J=7.0 Hz, 1H), 6.44 (d, J=3.1 Hz, 1H), 4.09 (s, 2H), 2.84 (t, J=7.7 Hz, 2H), 2.46 (t, J=7.7 Hz, 2H), 1.40 (s, 6H) ppm.

### Examples 282A and 282B

### (R) and (S) of 2-(3-(1-(2-(5-((4,6-difluoro-1H-indol-5-yl)oxy)-2-fluorophenyl)-1H-imidazol-5-yl)ethyl-2,2,2-d3)-2-fluorophenyl)acetic acid

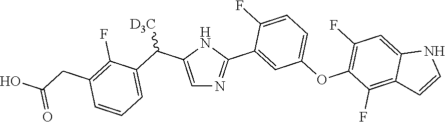 [SMILES: [2H]C([2H])([2H])C(c1cnc(-c2cc(Oc3c(F)cc4[nH]ccc4c3F)ccc2F)[nH]1)c1cccc(CC(=O)O)c1F]

### A. (R) and (S) of Ethyl 2-(3-(1-(2-(5-((4,6-difluoro-1H-indol-5-yl)oxy)-2-fluorophenyl)-1H-imidazol-5-yl)ethyl-2,2,2-d3)-2-fluorophenyl)acetate

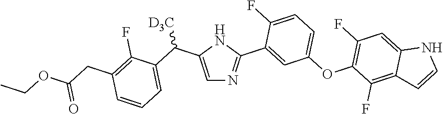 [SMILES: [2H]C([2H])([2H])C(c1cnc(-c2cc(Oc3c(F)cc4[nH]ccc4c3F)ccc2F)[nH]1)c1cccc(CC(=O)OCC)c1F]

To a stirred solution of 5-((4,6-difluoro-1H-indol-5-yl)oxy)-2-fluorobenzimidamide (Intermediate 24-9, 546 mg, 1.79 mmol) and sodium bicarbonate (231 mg, 2.75 mmol) in N,N-dimethylformamide (8 mL) was added ethyl (S)-2-(3-(4-bromo-3-oxobutan-2-yl-1,1,1-d3)-2-fluorophenyl)acetate (Intermediate 53, 460 mg, 1.05 mmol) dropwise at room temperature. The mixture was stirred at 75° C. for 2 hours, cooled to room temperature, and quenched with water (20 mL). The solution was extracted with ethyl acetate (3×20 mL). The combined organic layers were washed with water, brine, dried over sodium sulfate, filtered, and concentrated. The residue was purified by automated flash chromatography (40 g silica gel column, eluted with 50% ethyl acetate in petroleum ether) to give the title compound (190 mg, 27%) as a light-yellow oil. MS (ESI): 280 m/z [M+H]⁺. Chiral HPLC purity: 16% at 1.60 minutes (R enantiomer); 60% at 2.88 minutes (S enantiomer), with impurities. (Chiral SFC column conditions: S,S-WHELK-O1; 4.6×250 mm, 5 μM; Mobile phase: Carbon dioxide/methanol (0.2% 7M ammonia in methanol)=65:35; column temperature: 40° C.; Back Pressure: 120 bar; Flow rate: 4.0 mL/minute).

This light-yellow oil was subject to further SFC purification under the following conditions: Instrument: SFC-150 (Waters); Column: WHELK 20×250 mm, 10 μm; Column temperature: 35° C.; Mobile phase: carbon dioxide/methanol (0.2% 7M ammonia in methanol)=55/45; Flow rate: 100 g/minute; Back pressure: 100 bar; Detection wavelength: 214 nm; Cycle time: 4.5 minutes; Sample solution: 190 mg dissolved in 20 mL of methanol; Injection volume: 1 mL. The first eluent is ethyl (R)-2-(3-(1-(2-(5-((4,6-difluoro-1H-indol-5-yl)oxy)-2-fluorophenyl)-1H-imidazol-5-yl)ethyl-2,2,2-d3)-2-fluorophenyl)acetate (25 mg, 16%, white solid). The second eluent is ethyl (S)-2-(3-(1-(2-(5-((4,6-difluoro-1H-indol-5-yl)oxy)-2-fluorophenyl)-1H-imidazol-5-yl)ethyl-2,2,2-d3)-2-fluorophenyl)acetate (90 mg, 56%, white solid).

### B. (R)-2-(3-(1-(2-(5-((4,6-difluoro-1H-indol-5-yl)oxy)-2-fluorophenyl)-1H-imidazol-5-yl)ethyl-2,2,2-d3)-2-fluorophenyl)acetic acid

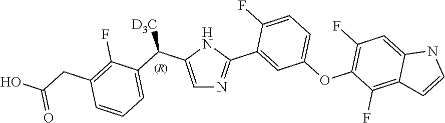

To a stirred solution of ethyl (R)-2-(3-(1-(2-(5-((4,6-difluoro-1H-indol-5-yl)oxy)-2-fluorophenyl)-1H-imidazol-5-yl)ethyl-2,2,2-d3)-2-fluorophenyl)acetate (25 mg, 0.044 mmol) in tetrahydrofuran/water/methanol (0.3 mL/0.1 mL/0.1 mL) was added lithium hydroxide monohydrate (3.9 mg, 0.0925 mmol). The reaction was stirred at room temperature for 2 hours, diluted with water (4 mL), and acidified with 1 M hydrochloric acid to pH˜4. The solution was extracted with ethyl acetate (3×5 mL). The combined organic layers were washed with brine, dried over sodium sulfate, filtered, and concentrated. The residue was purified by prep-HPLC to give the title compound (8.7 mg, 39%) as a white solid. MS (ESI): 513 m/z [M+H]⁺: Retention time: 1.52 minutes; Purity: >99% (214 nm). (LC-MS method: Mobile Phase: A: water (0.1% formic acid), B: Acetonitrile (0.1% formic acid); Gradient: 10% B increase to 90% B within 1.3 minutes; 90% B for 1.5 minutes, back to 5% B within 0.01 minutes; Flow Rate: 2 mL/minute; Column: Sunfire C18, 4.6*50 mm, 3.5 μm). Chiral HPLC purity: 98.4% (96.8% ee); Retention time: 2.89 minutes; (Chiral SFC conditions: (R,R)-WHELK-O1, 4.6×100 mm, 3.5 μm; Mobile phase: carbon dioxide/methanol (0.2% 7M ammonia in methanol)=70:30; Injection volume: 5 μL; Run time: 6 minutes; Flow rate: 3.0 mL/minute; Back pressure: 2000 psi; Column temperature: 40° C.). ¹H NMR (400 MHz, CD₃OD) δ 7.48-7.45 (m, 1H), 7.28 (d, J=3.2 Hz, 1H), 7.19-7.10 (m, 3H), 7.06-7.01 (m, 2H), 6.96-6.91 (m, 2H), 6.53 (d, J=3.2 Hz, 1H), 4.45 (s, 1H), 3.64 (s, 2H) ppm.

### C. (S)-2-(3-(1-(2-(5-((4,6-difluoro-1H-indol-5-yl)oxy)-2-fluorophenyl)-1H-imidazol-5-yl)ethyl-2,2,2-d3)-2-fluorophenyl)acetic acid

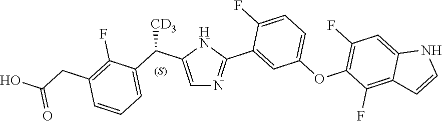

Utilizing identical conditions as described in Step B, ethyl (S)-2-(3-(1-(2-(5-((4,6-difluoro-1H-indol-5-yl)oxy)-2-fluorophenyl)-1H-imidazol-5-yl)ethyl-2,2,2-d3)-2-fluorophenyl)acetate (90 mg, 0.158 mmol) was hydrolyzed to the title compound (34 mg, 42%) as a white solid. MS (ESI): 513 m/z [M+H]⁺: Retention time: 1.52 minutes; Purity: >99% (214 nm) (LC-MS method: Mobile Phase: A: water (0.1% formic acid), B: Acetonitrile (0.1% formic acid); Gradient: 10% B increase to 90% B within 1.3 minutes; 90% B for 1.5 minutes, back to 5% B within 0.01 minutes; Flow Rate: 2 mL/minute; Column: Sunfire C18, 4.6*50 mm, 3.5 μm). Chiral HPLC purity: 98.4% (96.8% ee); Retention time: 2.89 minutes; (Chiral SFC conditions: (R,R)-WHELK-O1, 4.6×100 mm, 3.5 μm; Mobile phase: carbon dioxide/methanol (0.2% 7M ammonia in methanol)=70:30; Injection volume: 5 μL; Run time: 6 minutes; Flow rate: 3.0 mL/minute; Back pressure: 2000 psi; Column temperature: 40° C.). ¹H NMR (400 MHz, CD₃OD) δ 7.48-7.45 (m, 1H), 7.28 (d, J=3.2 Hz, 1H), 7.21-6.95 (m, 7H), 6.53 (d, J=3.2 Hz, 1H), 4.45 (s, 1H), 3.64 (s, 2H) ppm.

### Biological Assays

### Example 283: Aggregation Analysis Using Differential Static Light Scattering (DSLS)

Purified recombinant NBD1 was produced using previously described methods (A. Schmidt, J. L. Mendoza, P. J. Thomas (2011) Biochemical and Biophysical Approaches to Probe CFTR Structure (365-376) M. D. Amaral, K. Kunzelmann (eds.), Cystic Fibrosis, Methods in Molecular Biology 741, Springer Science+Business Media). The effect of test compounds on thermal stability of NBD1 was evaluated by differential static light scattering (DSLS) using the Harbinger Stargazer-384 instrument (Epiphyte Three, Toronto, Canada).

Test compounds were dissolved and diluted to desired concentrations in 100% DMSO. The compounds or DMSO controls (100 nL) were stamped into wells of a 385-well low volume optical plate (Corning Inc., Corning, NY) using the Echo 555 acoustic liquid handler (Labcyte Inc., San Jose, CA).

NBD1 protein was diluted to 0.2 mg/ml in S200 buffer (50 mM Tris-HCl, 150 mM NaCl, 5 mM MgCl₂, 2 mM ATP, 2 mM DTT, pH7.6) containing 1% glycerol. 10 uL of protein solution was aliquoted into the 384-well plate harboring the test compounds and 10 uL mineral oil was overlayed onto the protein solution, using the epMotion robotic liquid handler (Eppendorf North America, Hauppauge, NY). After placing into the Stargazer instrument, the plate was heated at 1° C. per minute to 70° C. Images were captured from 25° C. to 70° C. every 0.5° C. At the end of the experiment run, instrument software integrated image files and analyzed data automatically. A linear regression curve was generated for each well, representing the increase in light scattering over time. A temperature of aggregation (Tagg) was calculated based on the inflection point of the curve. To better compare data across experiments the average Tagg for DMSO control wells was calculated and subtracted from values for wells containing compounds to obtain a “ΔTagg” value. These ΔTagg values reflect stabilizing efficacy of the compounds. The data is shown in Table 5 below.

TABLE 5

| | DSLS |
|---|---|
| | NBD1/3D |
| | Human |
| | Stability |
| Example | delta-T (° C.) |
| No. | @ 100 (μM) |
| 1 | B |
| 2 | B |
| 3 | C |
| 4 | B |
| 5 | B |
| 6 | C |
| 7 | C |
| 8 | C |
| 9 | B |
| 10 | B |
| 11 | B |
| 12 | C |
| 13 | B |
| 14 | B |
| 15 | B |
| 16 | B |
| 17 | C |
| 18 | C |
| 19 | C |
| 20 | C |
| 21 | C |
| 22 | A |
| 23A | C |
| 23B | A |
| 24 | B |
| 25 | C |
| 26 | A |
| 27 | B |
| 28 | A |
| 29 | B |
| 30 | B |
| 31 | B |
| 32 | B |
| 33 | B |
| 34 | B |
| 35 | B |
| 36 | B |
| 37 | B |
| 38 | B |
| 39 | D |
| 40 | A |
| 41A | C |
| 41B | A |
| 42 | B |
| 43A | A |
| 43B | C |
| 44 | A |
| 45 | A |
| 46 | A |
| 47 | A |
| 48 | A |
| 49 | A |
| 50 | B |
| 51 | A |
| 52 | B |
| 53 | B |
| 54 | B |
| 55 | B |
| 56 | B |
| 57 | B |
| 58 | B |
| 59 | B |
| 60 | B |
| 61 | B |
| 62 | B |
| 63 | B |
| 64A | B |
| 64B | B |
| 65 | B |
| 66 | B |
| 67A | B |
| 67B | B |
| 68 | A |
| 69 | A |
| 70 | A |
| 71 | A |
| 72 | A |
| 73 | A |
| 74 | A |
| 75 | A |
| 76 | A |
| 77 | B |
| 78 | A |
| 79 | B |
| 80 | A |
| 81 | B |
| 82 | B |
| 83A | B |
| 83B | B |
| 84 | A |
| 85 | B |
| 86 | C |
| 87 | B |
| 88 | B |
| 89 | B |
| 90 | B |
| 91 | B |
| 92 | B |
| 93 | B |
| 94 | B |
| 95 | B |
| 96 | B |
| 97 | B |
| 98 | B |
| 99 | B |
| 100 | B |
| 101 | C |
| 102 | B |
| 103 | B |
| 104 | B |
| 105 | B |
| 106 | B |
| 107 | B |
| 108 | B |
| 109 | B |
| 110 | B |
| 111 | B |
| 112 | B |
| 113 | B |
| 114 | B |
| 115 | B |
| 116 | B |
| 117 | B |
| 118 | B |
| 119 | B |
| 120 | B |
| 121 | B |
| 122 | B |
| 123 | B |
| 124 | B |
| 125 | A |
| 126 | A |
| 127 | C |
| 128A | B |
| 128B | A |
| 129A | A |
| 129B | C |
| 130A | C |
| 130B | A |
| 131A | A |
| 131B | B |
| 132A | C |
| 132B | A |
| 133A | C |
| 133B | A |
| 134A | B |
| 134B | A |
| 135A | A |
| 135B | C |
| 136A | B |
| 136B | A |
| 136C | A |
| 136D | B |
| 137A | B |
| 137B | B |
| 137C | C |
| 137D | A |
| 138A | C |
| 138B | C |
| 138C | C |
| 138D | C |
| 139E | B |
| 139F | A |
| 139G | C |
| 139H | C |
| 140 | B |
| 141 | C |
| 142 | A |
| 143 | B |
| 144 | B |
| 145 | B |
| 146 | B |
| 147 | B |
| 148 | A |
| 149 | B |
| 150 | A |
| 151 | B |
| 152 | B |
| 153 | A |
| 154 | A |
| 155 | A |
| 156 | B |
| 157 | B |
| 158 | B |
| 159 | B |
| 160 | B |
| 161 | B |
| 162 | B |
| 163 | B |
| 164 | B |
| 165 | B |
| 166 | C |
| 167 | A |
| 168 | B |
| 169 | B |
| 170 | B |
| 171 | A |
| 172 | A |
| 173 | B |
| 174 | B |
| 175 | B |
| 176 | B |
| 177 | A |
| 178 | B |
| 179 | B |
| 180 | A |
| 181 | B |
| 182 | B |
| 183 | B |
| 184 | B |
| 185 | A |
| 186 | B |
| 187 | A |
| 188 | B |
| 189 | B |
| 190 | B |
| 191 | B |
| 192 | B |
| 193 | B |
| 194 | B |
| 195 | B |
| 196 | B |
| 197 | B |
| 198 | A |
| 199 | A |
| 200 | A |
| 201 | B |
| 202 | A |
| 203 | A |
| 204 | B |
| 205 | B |
| 206 | B |
| 281 | B |
| 207 | B |
| 208 | C |
| 209 | B |
| 210 | B |
| 211 | B |
| 212 | B |
| 213 | B |
| 214 | C |
| 215A | B |
| 215B | B |
| 216A | B |
| 216B | A |
| 217 | B |
| 218 | B |
| 219 | C |
| 220 | B |
| 221 | C |
| 222 | B |
| 223 | B |
| 224 | B |
| 225 | B |
| 226 | B |
| 227 | A |
| 228 | A |
| 229 | A |
| 230 | A |
| 231 | A |
| 232 | B |
| 233 | B |
| 234 | B |
| 235 | B |
| 236 | B |
| 237 | B |
| 238 | B |
| 239 | C |
| 240 | B |
| 241 | B |
| 242 | B |
| 243 | B |
| 244 | C |
| 245 | C |
| 246 | B |
| 247 | B |
| 248 | C |
| 249 | C |
| 250 | B |
| 251 | B |
| 252A | B |
| 252B | C |
| 253 | C |
| 254 | C |
| 255 | C |
| 256 | C |
| 257 | C |
| 258 | C |
| 259 | B |
| 260 | C |
| 261 | C |
| 262 | A |
| 263 | A |
| 264 | A |
| 265 | B |
| 266 | A |
| 267 | B |
| 268 | C |
| 269 | C |
| 270 | C |
| 271 | B |
| 272 | B |
| 273 | B |
| 274 | A |
| 275 | B |
| 276 | A |
| 277 | B |
| 278A | C |
| 278B | B |
| 279 | A |
| 280 | B |

A: T ≥ 10° C.;
B: 5-10° C.;
C: ≤5° C.;
D: Not Determined

### Example 284: TECC24 AUC Fold Over DMSO @ 10 μM

The effects of a test agent on CFTR-mediated transepithelial chloride transport was measured using TECC24 recording analysis. Test agents were solubilized in DMSO. Solubilized test agents were mixed with incubation medium containing DMEM/F12, Ultroser G (2%; Crescent Chemical, catalog #67042), Hyclone Fetal Clone II (2%; GE Healthcare, catalog #SH30066.02), bovine brain extract (0.25%; Lonza, catalog #CC-4098), insulin (2.5 μg/mL), IL-13 (10 ng/mL), hydrocortisone (20 nM), transferrin (2.5 μg/mL), triiodothyronine (500 nM), ethanolamine (250 nM), epinephrine (1.5 μM), phosphoethanolamine (250 nM), and retinoic acid (10 nM). Primary human bronchial epithelial cells from a ΔF508 homozygous CF donor (CF-HBE cells; from University of North Carolina Cystic Fibrosis Tissue Procurement Center), grown on Transwell HTS 24-well cell culture inserts (Costar, catalog #3378), were exposed to test agents or controls dissolved in incubation medium. The CF-HBE cells were cultured at 36.5° C. for 48 hours before TECC24 recordings were performed in the presence or absence of test agent, a positive control or vehicle (DMSO).

Following incubation, the transwell cell culture inserts containing the test agent or control-treated CF-HBE cells were loaded onto a TECC24 apparatus (TECC v7 or MTECC v2; EP Design) to record the transepithelial voltage (VT) and resistance (TEER) using 4 AgCl electrodes per well configured in current-clamp mode. The apical and basolateral bath solutions both contained (in mM) 140 NaCl, 5 KCl, 2 CaCl₂, 1 MgCl₂, 10 Hepes, and 10 glucose (adjusted to pH 7.4 with NaOH). To inhibit basal Na+ absorption, the ENaC inhibitor benzamil (10 μM) was added to the bath. Then, the adenylate cyclase activator, forskolin (10 μM), was added to the bath to activate CFTR. The forskolin-stimulated Cl— transport was halted by addition of CFTR inhibitor-172 (20 μM) to the bath at the end of the experiment to confirm specificity. VT and TEER recordings were digitally acquired at routine intervals using TECC or MTECC software (EP Design). VT and TEER were transformed into equivalent transpeithelial Cl— current (IEQ), and the Area Under the Curve (AUC) of the IEQ timecourse between forskolin and CFTR inhibitor-172 addition is generated using Excel (Microsoft). Efficacy is expressed as the ratio of the test agent AUC divided by vehicle AUC. EC50s based on AUC are generated using the non-linear regression log(agonist) vs. response function in Prism software (Graphpad) with HillSlope fixed=1.

If a test agent increased the AUC of the forskolin-stimulated IEQ relative to vehicle in CF-HBE cells, and this increase was inhibited by CFTR inhibitor-172, then the test agent was considered a CFTR corrector. The data is shown in Table 6 below.

TABLE 6

| Example | TECC24 @ | Example | TECC24 @ |
|---|---|---|---|
| No. | 10 μM over DMSO | No. | 10 uM over DMSO |
| 266 | C | 75 | A |
| 118 | B | 74 | A |
| 86 | C | 67B | C |
| 140 | C | 277 | A |
| 77 | C | 119 | C |
| 272 | B | 121 | A |
| 273 | C | 128B | A |
| 40 | A | 129A | A |
| 194 | C | 252A | A |
| 97 | C | 124 | B |
| 123 | B | 280 | A |
| 205 | B | 278B | A |
| 274 | C | 43A | A |
| 77 | A | 130B | A |
| 159 | B | 270 | C |
| 52 | B | 180 | A |
| 206 | C | 279 | A |
| 167 | C | 131A | A |
| 171 | C | 41B | A |
| 276 | C | 132B | A |
| 169 | C | 23B | A |
| 45 | B | 125 | A |
| 155 | A | 133B | C |
| 170 | C | 84 | B |
| 64A | B | 126 | B |
| 64B | A | 134B | A |
| 80 | C | 136C | A |
| 135A | C | 137D | A |
| 139F | B | | |

ND = Not determined;
“A” refers to AUC @10 uM >= 12;
“B” refers to AUC @10 uM between 4-12;
“C” refers to AUC @ 10 uM <= 4.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

What is claimed is:

1. A compound of Formula I: 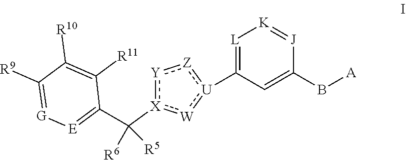
wherein
A is selected from 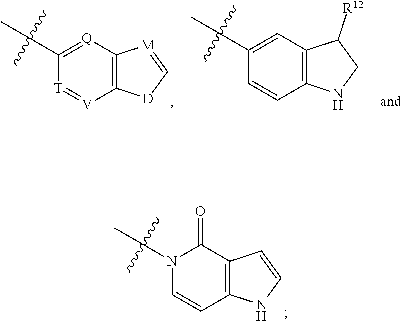
B is O or C═O;
M is CR¹³ or N;
Q is CR¹ or N;
T is CR² or N;
V is CR³ or N;
D is NR¹⁴ or S;
J is CR^{4a} or N;
K is CR^{4b} or N;
L is CR^{4c} or N;
E is CR⁷ or N;
G is CR⁸ or N;
U is N or C;
W is N, NR^{a}, O or S;
X is N or C;
Y is CR^{b}, N or NR^{c};
Z is N, NR^{d}, O, S, or CH; [symbol]
is a single or double bond;
R^{a}, R^{c}, and R^{d} are each independently selected from H, alkyl, or aralkyl;
R^{b} is selected from H, halo, or alkyl;
R¹ is selected from H, D, halo, CN, nitro, alkyl, alkenyl, alkynyl, alkoxy, hydroxy, amido, cycloalkyl, aryl, heterocyclyl, heteroaryl, alkylcarbonyl, carboxy, alkoxycarbonyl, amino, amido, phosphino, thioalkyl, sulfinyl, sulfonyl, sulfonimidolyl, phosphoryl, sulfonamido, and isothiazolidine-1,1-dioxide;
R² is selected from H, halo, CN, alkyl, alkoxy, hydroxy, amino, alkoxycarbonyl, and amido;
R³ is selected from H, halo, CN, and alkyl;
R^{4a}, R^{4b}, and R^{4c} are each independently selected from H, halo, CN, alkyl, alkoxy, hydroxy, amino, alkoxycarbonyl, and amido;
R⁵ is selected from H, halo, CN, alkyl, alkenyl, alkynyl, hydroxy, alkoxy, alkylcarbonyl, alkoxycarbonyl, amino, carboxy, and amido;
R⁶ is H, CN or alkyl; or
R⁵ and R⁶ together with the carbon atoms to which they are attached form a vinyl, carbonyl, cycloalkyl or heterocyclyl;
R⁷ and R¹¹ are each independently selected from H, halo, CN, alkyl, alkoxy, aryl, and heteroaryl; or
R⁶ and R⁷ together with the carbon atoms to which they are attached form a cycloalkyl;
R⁸ and R¹⁰ are each independently selected from H, halo, alkyl, alkenyl, alkynyl, alkoxy, amido, carboxy, alkoxycarbonyl, thioalkyl, cycloalkyl, and heterocyclyl; or
R⁷ and R⁸ together with the carbon atoms to which they are attached form a cycloalkyl;
R⁹ is selected from H, halo, alkyl, alkynyl, and alkoxy; or
R⁸ and R⁹ together with the carbon atoms to which they are attached form a cycloalkyl, heteroaryl, or heterocyclyl; or
R⁹ and R¹⁰ together with the carbon atoms to which they are attached form a cycloalkyl;
R¹² is H, deuterium or carboxy;
R¹³ is H, deuterium, halo or thioalkyl;
R¹⁴ is H, sulfonyl, carbonyl or phosphate;
wherein each alkyl, alkenyl, alkynyl, alkoxy, amino, amido, alkylcarbonyl, alkoxycarbonyl, cycloalkyl, heterocyclyl, aryl, and heteroaryl is independently unsubstituted or substituted with one or more substituents selected from deuterium, halo, CN, nitro, hydroxy, alkyl, amino, amido, oxo, boronate, cycloalkyl, aryl, heterocyclyl, heteroaryl, carbonyl, carboxy, acyloxy, alkoxy, thio, phosphino, phosphono, phosphate, sulfinyl, sulfonyl, sulfonamido, ureido, amidino, hydroxyamidino, iminosulfanoyl, 2-tetrahydrothiophene-1,1-dioxide, and isothiazolidine-1,1-dioxide; and
wherein at least one of R⁷, R⁸, R⁹, R¹⁰, and R¹¹ is not H; and provided that when E is CR⁷, G is CR⁸, and one of R⁷, R⁸, R⁹, R¹⁰, and R¹¹ is unsubstituted alkyl, halo, or haloalkyl, then the ring to which R⁷, R⁸, R⁹, R¹⁰, and R¹¹ are attached is disubstituted.

2. The compound of claim 1, wherein A is selected from

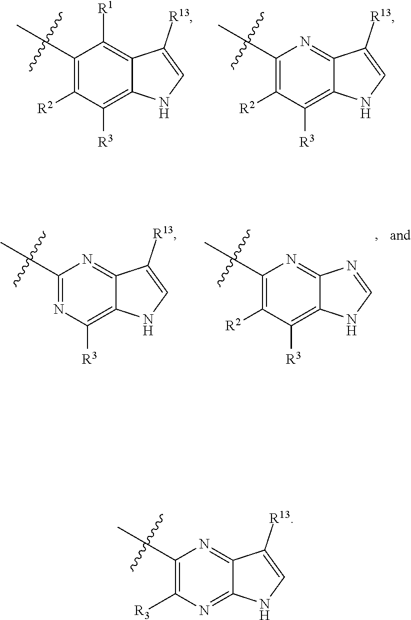

3. The compound of claim 1 or 2, wherein
W is NH;
X is C;
Y is selected from CH, C-Me, and C—CMe₂OH; and
Z is N.

4. The compound of claim 1 or 2, wherein
W is NH;
X is C;
Y is N; and
Z is N.

5. The compound of claim 1 or 2, wherein
W is N;
X is N;
Y is CH; and
Z is CH.

6. The compound of claim 1 or 2, wherein
W is N;
X is C;
Y is CH; and
Z is S.

7. The compound of claim 1 or 2, wherein
W is N;
X is C;
Y is CH; and
Z is S.

8. The compound of claim 1 or 2, wherein
W is N;
X is C;
Y is CH; and
Z is CH or C-Me

9. The compound of claim 1 or 2, wherein
W is N;
X is N;
Y is CH; and
Z is CH.

10. The compound of claim 1 or 2, wherein
W is N;
X is N;
Y is CH; and
Z is NH.

11. The compound of claim 1 or 2, wherein
U is N;
W is N;
X is C;
Y is CH; and
Z is CH.

12. The compound of any one of claims 1-11, wherein
A is 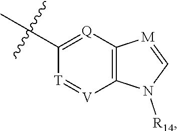
M is CH or C-deuterium;
Q is CR¹;
T is CR²;
V is CR³;
R¹ is selected from H, halo, alkyl, amido, phosphino, sulfonyl and sulfonamide;
R² is H or halo;
R³ is H or halo;
R^{4c} is H or halo; and
R¹⁴ is H or phosphate.

13. The compound of claim 12, wherein
R¹ is methyl;
R² is F;
R³ is H; and
R^{4c} is F.

14. The compound of claim 12, wherein
R¹ is methyl;
R² is F;
R³ is F; and
R^{4c} is F.

15. The compound of claim 12, wherein
R¹ is CH₂CHF₂;
R² is F;
R³ is H; and
R^{4c} is F.

16. The compound of claim 12, wherein
R¹ is CH₂CF₃;
R² is F;
R³ is H; and
R^{4c} is F.

17. The compound of claim 12, wherein
R¹ is F;
R² is F;
R³ is H; and
R^{4c} is F.

18. The compound of claim 12, wherein
R¹ is F;
R² is F;
R³ is F; and
R^{4c} is F.

19. The compound of claim 12, wherein
R¹ is F;
R² is F;
R³ is H; and
R^{4c} is H.

20. The compound of claim 19, wherein R^{4a} is deuterium.

21. The compound of claim 12, wherein
R¹ is H;
R² is H;
R³ is H; and
R^{4c} is F.

22. The compound of claim 12, wherein
R¹ is —PO(Me)₂;
R² is F;
R³ is H; and
R^{4c} is F.

23. The compound of claim 12, wherein
R¹ is —PO(Me)₂;
R² is F;
R³ is H; and
R^{4c} is H.

24. The compound of claim 12, wherein
R¹ is —SO₂Me;
R² is F;
R³ is H; and
R^{4c} is F.

25. The compound of claim 12, wherein
R¹ is —SO₂Me;
R² is F;
R³ is H; and
R^{4c} is H.

26. The compound of claim 12, wherein
R¹ is —SO₂Me;
R² is F;
R³ is F; and
R^{4c} is F.

27. The compound of claim 12, wherein
R¹ is —SONHMe;
R² is F;
R³ is H; and
R^{4c} is F.

28. The compound of claim 12, wherein
R¹ is H;
R² is H;
R³ is H; and
R^{4c} is F.

29. The compound of claim 12, wherein
R¹ is —CONHMe;
R² is F;
R³ is H; and
R^{4c} is F.

30. The compound of claim 12, wherein
R¹ is —CH₂SONHMe;
R² is F;
R³ is H; and
R^{4c} is F.

31. The compound of claim 12, wherein
R¹ is —CH₂SO₂Me;
R² is F;
R³ is H; and
R^{4c} is F.

32. The compound of claim 12, wherein
R¹ is —CH₂CH₂SO₂Me;
R² is F;
R³ is H; and
R^{4c} is F.

33. The compound of claim 12, wherein
R¹ is —CH₂CH₂POMe₂;
R² is F;
R³ is H; and
R^{4c} is F.

34. The compound of claim 12, wherein
R¹ is —CH₂POMe₂;
R² is F;
R³ is H; and
R^{4c} is F.

35. The compound of claim 12, wherein
R¹ is 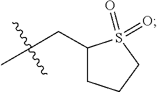 [SMILES: *CC1CCCS1(=O)=O]
R² is F;
R³ is H; and
R^{4c} is F.

36. The compound of claim 12, wherein
R¹ is 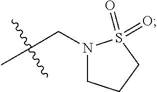 [SMILES: *CN1CCCS1(=O)=O]
R² is F;
R³ is H; and
R^{4c} is F.

37. The compound of claim 12, wherein R¹ is alkyl and is unsubstituted or substituted with one or more substituents selected from halo, sulfonyl, phosphino, 2-tetrahydrothiophene-1,1-dioxide, and isothiazolidine-1,1-dioxide.

38. The compound of claim 1, wherein
J is CR^{4a};
K is CR^{4b};
L is CR^{4c}; and
each of R^{4a}, R^{4b}, and R^{4c} is independently selected from H, D, and halo.

39. The compound of claim 38, wherein
R^{4a} is H;
R^{4b} is H; and
R^{4c} is F.

40. The compound of any one of claims 1-39, wherein
R⁵ is selected from H, alkyl, amino and hydroxy;
R⁶ is H, CN or alkyl; or
R⁵ and R⁶ together with the carbon atoms to which they are attached form a cycloalkyl or a heterocyclyl.

41. The compound of claim 40, wherein each alkyl, cycloalkyl and heterocyclyl is independently unsubstituted or substituted with one or more substituents selected from D, CN, halo, hydroxy, and alkoxy.

42. The compound of claim 41, wherein
R⁵ is selected from H, methyl, CD₃, t-butyl, —CH₂CN, —CH₂OCH₃, —(CH₂)₂O—(CH₂)₂O—CHMe₂, —NHMe, and hydroxy;
R⁶ is selected from H, methyl, —CHF₂, CF₃, and CN; or
R⁵ and R⁶ together with the carbon atoms to which they are attached form a

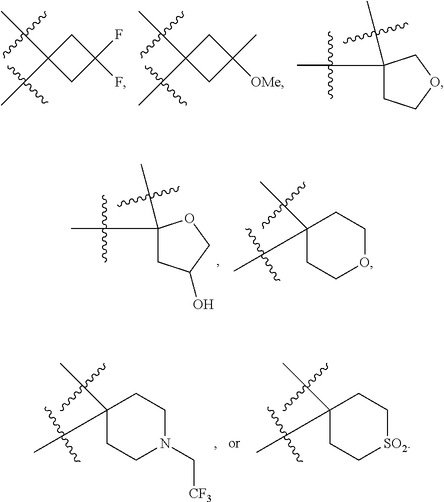

43. The compound of any one of claims 1-42, wherein
E is CR⁷;
G is CR⁸;
R⁷ is H or halo;
R⁸ is alkyl or cycloalkyl, each unsubstituted or substituted with carboxy;
R⁹ is H;
R¹⁰ is H or halo; and
R¹¹ is H.

44. The compound of claim 43, wherein
R⁷ is H;
R⁸ is —CH₂CH₂COOH;
R⁹ is H;
R¹⁰ is H; and
R¹¹ is H.

45. The compound of claim 43, wherein
R⁷ is F;
R⁸ is —CH₂CH₂COOH;
R⁹ is H;
R¹⁰ is H; and
R¹¹ is H.

46. The compound of claim 43, wherein
R⁷ is F;
R⁸ is —CH₂CH₂COOH;
R⁹ is H;
R¹⁰ is F; and
R¹¹ is H.

47. The compound of claim 43, wherein
R⁷ is F;
R⁸ is —CH₂CH₂COOH;
R⁹ is H;
R¹⁰ is H; and
R¹¹ is F.

48. The compound of claim 43, wherein
R⁷ is F;
R⁸ is —CH₂CH₂COOH;
R⁹ is H;
R¹⁰ is H; and
R¹¹ is H.

49. The compound of claim 43, wherein
R⁷ is F;
R⁸ is —CH₂CHMeCOOH;
R⁹ is H;
R¹⁰ is H; and
R¹¹ is H.

50. The compound of claim 43, wherein
R⁷ is F;
R⁸ is —(CH₂)₃OPO(OH)₂;
R⁹ is H;
R¹⁰ is H; and
R¹¹ is H.

51. The compound of claim 43, wherein
R⁷ is F;
R⁸ is 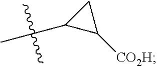 [SMILES: *C1CC1C(=O)O]
R⁹ is H;
R¹⁰ is H; and
R¹¹ is H.

52. The compound of claim 42, wherein
R⁷ is H;
R⁸ is —CH₂CH₂OH;
R⁹ is H;
R¹⁰ is H; and
R¹¹ is H.

53. The compound of any one of claims 1-42, wherein
E is N;
R⁸ is —CH₂CH₂COOH;
R⁹ is H;
R¹⁰ is H; and
R¹¹ is H.

54. The compound of claim 1 or 2, wherein the compound of Formula I is a compound of Formula IA: 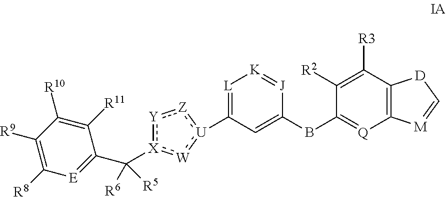
wherein
B is O or C═O;
M is CR¹³;
Q is CR¹ or N;
T is CR²;
V is CR³;
D is NR¹⁴ or S;
J is CR^{4a};
K is CR^{4b} or N;
L is CR^{4c};
E is CR⁷ or N;
U is N or C;
W is N, NR^{a}, or S;
X is N or C;
Y is CR^{b}, N or NR^{c};
Z is N, NR^{d}, O, S, or CH; [symbol]
is a single or double bond;
R^{a}, R^{c}, and R^{d} are each independently selected from H, alkyl, or aralkyl;
R^{b} is selected from H, halo, or alkyl;
R¹ is selected from H, halo, alkyl, alkenyl, amido, cycloalkyl, heterocyclyl, heteroaryl, phosphino, thioalkyl, sulfinyl, and sulfonyl;
R² is selected from H and halo;
R³ is selected from H or halo;
R^{4a}, R^{4b}, and R^{4c} are each independently selected from H and haloalkyl;
R⁵ is selected from H, CN, alkyl, hydroxy, alkoxy, and amino;
R⁶ is H or alkyl; or
R⁵ and R⁶ together with the carbon atoms to which they are attached form a vinyl, carbonyl, cycloalkyl or heterocyclyl;
R⁷ and R¹¹ are each independently selected from H and halo; or
R⁸ and R¹⁰ are each independently selected from H, halo, alkyl, alkenyl, thioalkyl, and cycloalkyl; or
R⁷ and R⁸ together with the carbon atoms to which they are attached form a cycloalkyl;
R⁹ is selected from H, halo, alkyl, and alkynyl; or
R⁸ and R⁹ together with the carbon atoms to which they are attached form a cycloalkyl, heteroaryl, and heterocyclyl;
R¹³ is H, deuterium, or halo;
R¹⁴ is H or phosphate;
wherein each alkyl, alkenyl, alkynyl, alkoxy, amino, amido, cycloalkyl, heterocyclyl, and heteroaryl is independently unsubstituted or substituted with one or more substituents selected from deuterium, halo, CN, hydroxy, alkyl, amino, amido, cycloalkyl, heterocyclyl, heteroaryl, carbonyl, carboxy, alkoxy, phosphino, phosphate, sulfonyl, sulfonamido, iminosulfanoyl, and 2-tetrahydrothiophene-1,1-dioxide; and
wherein at least one of R⁷, R⁸, R⁹, R¹⁰, and R¹¹ is not H.

55. The compound of claim 54, wherein
B is O or C═O;
M is CR¹³;
Q is CR¹ or N;
T is CR²;
V is CR³;
D is NR¹⁴ or S;
J is CR^{4a};
K is CR^{4b} or N;
L is CR^{4c};
E is CR⁷ or N;
U is N or C;
W is N or NR^{a};
X is N or C;
Y is CR^{b} or N;
Z is N, NR^{d}, O, S, or CH; [symbol]
is a single or double bond;
R^{a} is H;
R^{b}, R^{c}, and R^{d} are each independently H or alkyl;
R¹ is selected from H, halo, alkyl, amido, phosphino, and sulfonyl;
R² is selected from H and halo;
R³ is selected from H or halo;
R^{4a}, R^{4b}, and R^{4c} are each independently selected from H, halo, and alkyl;
R⁵ is selected from H, CN, alkyl, hydroxy, and amino;
R⁶ is H or alkyl; or
R⁵ and R⁶ together with the carbon atoms to which they are attached form a cycloalkyl or a heterocyclyl;
R⁷ and R¹¹ are each independently selected from H and halo; or
R⁹ is selected from H and alkyl;
R¹³ is H or deuterium;
R¹⁴ is H or phosphate;
wherein each alkyl, alkoxy, amino, amido, cycloalkyl, heterocyclyl, and heteroaryl is independently unsubstituted or substituted with one or more substituents selected from deuterium, halo, CN, hydroxy, alkyl, carboxy, alkoxy, phosphino, phosphate, sulfonyl, sulfonamido, iminosulfanoyl, and 2-tetrahydrothiophene-1,1-dioxide; and
wherein at least one of R⁷, R⁸, R⁹, R¹⁰, and R¹¹ is not H.

56. The compound of claim 1 or 2, wherein the compound of Formula I is a compound of Formula IB: 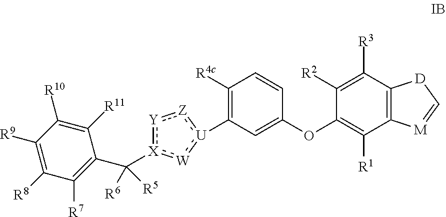
wherein
U is N or C;
W is N or NR^{a}, where R^{a} is H;
X is N or C;
Y is N or CR^{b}, where R^{b} is H or alkyl;
Z is N, NR^{d}, O or S, where R^{d} is H or alkyl;
M is CH or C-deuterium;
D is NR¹⁴; [symbol]
is a single or double bond;
R¹ is H, halo, alkyl, amido, sulfonyl and sulfonamido;
R² is H or halo;
R³ is H or halo;
R^{4c} is H or halo;
R⁵ is selected from H, CN, alkyl, hydroxy, and amino;
R⁶ is H, alkyl, or hydroxy; or
R⁵ and R⁶ together with the carbon atoms to which they are attached form a heterocyclyl;
R⁷ is H or halo;
R⁸ is alkyl or cycloalkyl;
R⁹ is H;
R¹⁰ and R¹¹ are each H or halo; and
R¹⁴ is H or phosphate;
wherein each alkyl, amino, alkoxy. alkylcarbonyl, cycloalkyl, and heterocyclyl is independently unsubstituted or substituted with one or more substituents selected from deuterium, halo, CN, hydroxy, alkyl, alkoxy, carboxy, sulfonyl, sulfonamido, phosphino, phosphate, iminosulfamoyl, 2-tetrahydrothiophene-1,1-dioxide, and isothiazolidine-1,1-dioxide.

57. The compound of claim 56, wherein
U is C;
W is N or NR^{a}, where R^{a} is H;
X is N or C;
Y is N, CR^{b}, where R^{b} is H or alkyl;
Z is N, NR^{d}, or O, where R^{d} is H or alkyl;
M is CH or C-deuterium;
D is NR¹⁴;
R¹ is selected from halo, alkyl, sulfonyl and sulfonamido;
R² is H or halo;
R³ is H or halo;
R^{4c} is halo;
R⁵ is selected from H, CN, and alkyl,
R⁶ is H or alkyl,
R⁷ is H or halo;
R⁸ is alkyl;
R⁹ is H;
R¹⁰ is H or halo;
R¹¹ is H; and
R¹⁴ is H or phosphate;
wherein each alkyl is independently unsubstituted or substituted with one or more substituents selected from deuterium, halo, carboxy, sulfonyl, phosphino, phosphate, iminosulfamoyl, tetrahydrothiophene-1,1-dioxide and isothiazolidine-1,1-dioxide.

58. The compound of claim 56, wherein
U is C;
W is N or NR^{a}, where R^{a} is H;
X is N or C;
Y is CR^{b}, where R^{b} is H;
Z is N, or NR^{d}, where R^{a} is H;
M is CH;
R¹ is halo or sulfonyl;
R² is halo;
R³ is H or halo;
R^{4c} is halo;
R⁵ is H, or alkyl,
R⁶ is H,
R⁷ is H or halo;
R⁸ is alkyl substituted with carboxy;
R⁹ is H; and
R¹⁰ is H or halo; and
R¹¹ is H.

59. The compound of claim 1, wherein the compound of Formula I is a compound of Formula IC: 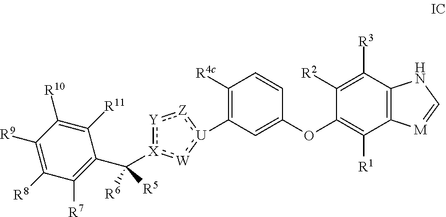
wherein
R⁵ is selected from halo, alkyl, alkenyl, alkynyl, hydroxy, alkoxy, alkylcarbonyl, alkoxycarbonyl, amino, and amido; and
R⁶ is H or CN.

60. The compound of claim 1, wherein the compound of Formula I is a compound of Formula ID: 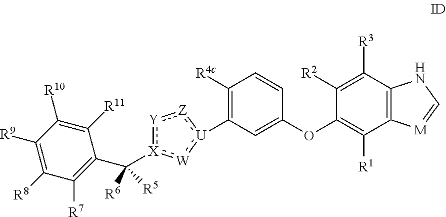
R⁵ is selected from halo, alkyl, alkenyl, alkynyl, hydroxy, alkoxy, alkylcarbonyl, alkoxycarbonyl, amino, and amido; and
R⁶ is H or CN.

61. The compound of claim 59 or 60, wherein R⁵ is methyl or hydroxy.

62. The compound of claim 1, wherein the compound of Formula I is a compound of Formula IE: 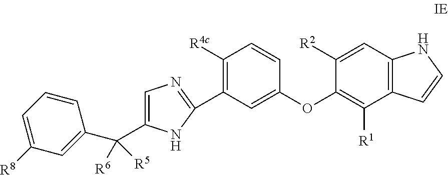
R¹ is H or F;
R² is H or F;
R^{4c} is H or F;
R⁵ is H, Me or OH;
R⁶ is H or Me; or
R⁵ and R⁶ together with the carbon atoms to which they are attached form a cyclobutyl, cyclopentyl, tetrahydropyran, or dioxylanyl; and
R⁸ is selected from carboxyalkyl, hydroxyalkyl, alkoxycarbonylalkyl, amidoalkyl, and cyclopropyl.

63. A compound of Formula I′: 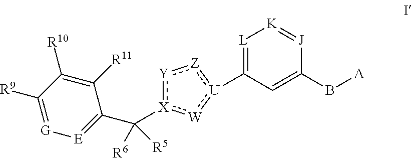
wherein
A is selected from 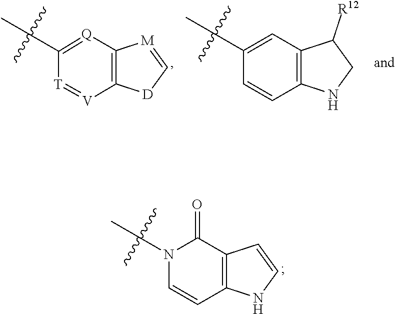
B is selected from O, C═O, C(R^{e})₂, N, S, SO, and SO₂;
M is CR¹³ or N;
Q is CR¹ or N;
T is CR² or N;
V is CR³ or N;
D is NR¹⁴ or S;
J is CR^{4a} or N;
K is CR^{4b} or N;
L is CR^{4c} or N;
E is CR⁷ or N;
G is CR⁸ or N;
U is N or C;
W is N, NR^{a}, O or S;
X is N or C;
Y is CR^{b}, N or NR^{c};
Z is N, NR^{d}, O, S, CH or C-alkyl; [symbol]
is a single or double bond;
R^{a}, R^{c}, and R^{d} are each independently selected from H, alkyl, or aralkyl;
R^{b} is selected from H, halo, or alkyl;
each R^{e} is independently selected from H, halo and hydroxy; wherein both values for R^{e} are not simultaneously H;
R¹ is selected from H, D, halo, CN, nitro, alkyl, alkenyl, alkynyl, alkoxy, hydroxy, amido, cycloalkyl, aryl, heterocyclyl, heteroaryl, alkylcarbonyl, carboxy, alkoxycarbonyl, amino, amido, phosphino, thioalkyl, sulfinyl, sulfonyl, sulfonimidolyl, phosphoryl, sulfonamido, and isothiazolidine-1,1-dioxide;
R² is selected from H, halo, CN, alkyl, alkoxy, hydroxy, amino, alkoxycarbonyl, and amido;
R³ is selected from H, halo, CN, and alkyl;
R^{4a}, R^{4b}, and R^{4c} are each independently selected from H, halo, CN, alkyl, alkoxy, hydroxy, amino, alkoxycarbonyl, and amido;
R⁵ is selected from H, halo, CN, alkyl, alkenyl, alkynyl, hydroxy, alkoxy, alkylcarbonyl, alkoxycarbonyl, amino, carboxy, and amido;
R⁶ is H, CN or alkyl; or
R⁵ and R⁶ together with the carbon atoms to which they are attached form a vinyl, carbonyl, carboxy, cycloalkyl or heterocyclyl;
R⁷ and R¹¹ are each independently selected from H, halo, CN, alkyl, alkoxy, aryl, and heteroaryl; or
R⁶ and R⁷ together with the carbon atoms to which they are attached form a cycloalkyl or heterocyclyl;
R⁸ and R¹⁰ are each independently selected from H, halo, alkyl, alkenyl, alkynyl, alkoxy, amido, carboxy, alkoxycarbonyl, thioalkyl, cycloalkyl, and heterocyclyl; or
R⁷ and R⁸ together with the carbon atoms to which they are attached form a cycloalkyl or a heterocyclyl;
R⁹ is selected from H, halo, alkyl, alkynyl, and alkoxy; or
R⁸ and R⁹ together with the carbon atoms to which they are attached form a cycloalkyl, heteroaryl, or heterocyclyl; or
R⁹ and R¹⁰ together with the carbon atoms to which they are attached form a cycloalkyl or heterocyclyl;
R¹² is H, deuterium or carboxy;
R¹³ is H, deuterium, halo or thioalkyl;
R¹⁴ is H, sulfonyl, carbonyl or phosphate;
wherein each alkyl, alkenyl, alkynyl, alkoxy, amino, amido, alkylcarbonyl, alkoxycarbonyl, cycloalkyl, heterocyclyl, aryl, and heteroaryl is independently unsubstituted or substituted with one or more substituents selected from deuterium, halo, CN, nitro, hydroxy, alkyl, amino, amido, oxo, boronate, cycloalkyl, aryl, heterocyclyl, heteroaryl, carbonyl, carboxy, acyloxy, alkoxy, thio, phosphino, phosphono, phosphate, sulfinyl, sulfonyl, sulfonamido, ureido, amidino, hydroxyamidino, iminosulfanoyl, 2-tetrahydrothiophene-1,1-dioxide, and isothiazolidine-1,1-dioxide;
wherein at least one of R⁷, R⁸, R⁹, R¹⁰, and R¹¹ is not H; and provided that when E is CR⁷, G is CR⁸, and one of R⁷, R⁸, R⁹, R¹⁰, and R¹¹ is unsubstituted alkyl, halo, or haloalkyl, then the ring to which R⁷, R⁸, R⁹, R¹⁰, and R¹¹ are attached is disubstituted.

64. A compound selected from any of the compounds shown in Table 1.

65. The compound of any one of claims 1-64, wherein the compound is a CFTR corrector.

66. A pharmaceutical composition comprising a compound of any one of the preceding claims, and one or more pharmaceutically acceptable carriers or excipients.

67. The pharmaceutical composition of claim 66, further comprising one or more CFTR therapeutic agents.

68. A method of treating deficient CFTR activity in a cell, comprising contacting the cell with a compound of any one of claims 1-65, or the pharmaceutical composition of claim 66 or 67.

69. The method of claim 68, wherein contacting the cell occurs in a subject in need thereof, thereby treating a CFTR-mediated condition and/or disease.

70. The method of claim 69, wherein the disease or condition is selected from cystic fibrosis, asthma, smoke induced COPD, chronic bronchitis, rhinosinusitis, constipation, pancreatitis, pancreatic insufficiency, male infertility caused by congenital bilateral absence of the vas deferens (CBAVD), mild pulmonary disease, idiopathic pancreatitis, allergic bronchopulmonary aspergillosis (ABPA), liver disease, hereditary emphysema, hereditary hemochromatosis, coagulation-fibrinolysis deficiencies, protein C deficiency, Type 1 hereditary angioedema, lipid processing deficiencies, familial hypercholesterolemia, Type 1 chylomicronemia, abetalipoproteinemia, lysosomal storage diseases, I-cell disease/pseudo-Hurler, mucopolysaccharidoses, Sandhof/Tay-Sachs, Crigler-Najjar type II, polyendocrinopathy/hyperinsulemia, Diabetes mellitus, Laron dwarfism, myleoperoxidase deficiency, primary hypoparathyroidism, melanoma, glycanosis CDG type 1, congenital hyperthyroidism, osteogenesis imperfecta, hereditary hypofibrinogenemia, ACT deficiency, Diabetes insipidus (DI), neurophyseal DI, neprogenic DI, Charcot-Marie Tooth syndrome, Perlizaeus-Merzbacher disease, neurodegenerative diseases, Alzheimer's disease, Parkinson's disease, amyotrophic lateral sclerosis, progressive supranuclear plasy, Pick's disease, several polyglutamine neurological disorders, Huntington's, spinocerebullar ataxia type I, spinal and bulbar muscular atrophy, dentatorubal pallidoluysian, myotonic dystrophy, spongiform encephalopathies, hereditary Creutzfeldt-Jakob disease, Fabry disease, Straussler-Scheinker syndrome, COPD, dry-eye disease, Sjogren's disease, Osteoporosis, Osteopenia, bone healing and bone growth, bone repair, bone regeneration, reducing bone resorption, increasing bone deposition, Gorham's Syndrome, chloride channelopathies, myotonia congenita, Bartter's syndrome type III, Dent's disease, hyperekplexia, epilepsy, hyperekplexia, lysosomal storage disease, Angelman syndrome, Primary Ciliary Dyskinesia (PCD), PCD with situs inversus, PCD without situs inversus and ciliary aplasia.

71. The method of claim 69 or 70, wherein the disease or condition is selected from cystic fibrosis, congenital bilateral absence of vas deferens (CBAVD), acute, recurrent, or chronic pancreatitis, disseminated bronchiectasis, asthma, allergic pulmonary aspergillosis, chronic obstructive pulmonary disease (COPD), chronic sinusitis, dry eye disease, protein C deficiency, Abetalipoproteinemia, lysosomal storage disease, type 1 chylomicronemia, mild pulmonary disease, lipid processing deficiencies, type 1 hereditary angioedema, coagulation-fibrinolyis, hereditary hemochromatosis, CFTR-related metabolic syndrome, chronic bronchitis, constipation, pancreatic insufficiency, hereditary emphysema, and Sjogren's syndrome.

72. The method of any one of claims 69-71, wherein the disease or condition is cystic fibrosis.

73. A method of treating cystic fibrosis in a subject, comprising administering to the subject a therapeutically effective amount of a compound of any one of claims 1-65, or the pharmaceutical composition of claim 66 or 67.

74. The method of claim 73, wherein the subject is human.

75. The method according to claim 73 or 74, wherein said subject is at risk of developing cystic fibrosis, and wherein said administering step is carried out prior to the onset of symptoms of cystic fibrosis in said subject.
